(19) United States
(12) Patent Application Publication
(10) Pub. No.: US 2023/0078199 A1
(43) Pub. Date: Mar. 16, 2023

(54) COMPOSITION AND ORGANIC LIGHT-EMITTING DEVICE INCLUDING THE SAME

(71) Applicant: Samsung Electronics Co., Ltd., Suwon-si (KR)

(72) Inventors: Sooghang Ihn, Hwaseong-si (KR); Eunsuk Kwon, Suwon-si (KR); Yusuke MARUYAMA, Hwaseong-si (KR); Sangmo Kim, Hwaseong-si (KR); Jong Soo Kim, Hanam-si (KR); Sungho Nam, Daegu (KR); Hasup Lee, Seoul (KR); Jun Chwae, Seoul (KR)

(21) Appl. No.: 17/569,083

(22) Filed: Jan. 5, 2022

(30) Foreign Application Priority Data
Jan. 5, 2021 (KR) 10-2021-0001063
Jan. 3, 2022 (KR) 10-2022-0000511

(51) Int. Cl. H01L 51/00; C07D 251/24; C07F 5/02; C07D 209/86; C09K 11/06

(52) U.S. Cl. CPC H01L 51/0072; C07D 251/24; H01L 51/0067; H01L 51/0054; H01L 51/5028; H01L 51/008; C07D 209/86; C09K 11/06; C07F 5/027

(57) ABSTRACT

A composition comprising a first compound, a second compound, and a third compound, wherein the first compound, the second compound, and the third compound are different from each other, the first compound satisfies one of Condition 1 and Condition 2 as described herein, and the second compound includes a compound represented by Formula 1:

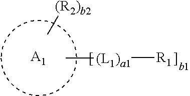

wherein ring A₁ is a condensed cyclic group in which 3 or more cyclic groups are condensed with each other, and the 3 or more cyclic groups are each a C₅-C₃₀ carbocyclic group or a C₁-C₃₀ heterocyclic group; a1 is an integer from 1 to 5; b1 is an integer from 3 to 10; and L₁, R₁, and R₂ are as described herein.

### CROSS-REFERENCE TO RELATED APPLICATION

This application claims priority to and the benefits under 35 U.S.C. § 119 of Korean Patent Application Nos. 10-2021-0001063, filed on Jan. 5, 2021, and 10-2022-0000511, filed on Jan. 3, 2022, both in the Korean Intellectual Property Office, the entire contents of which are incorporated by reference herein.

### BACKGROUND

### 1. Field

One or more embodiments relate to compositions and organic light-emitting devices including the same.

### 2. Description of the Related Art

Organic light-emitting devices (OLEDs) are self-emission devices that produce full-color images. Relative to other electronic devices, OLEDs can provide full color images that have wide viewing angles, high contrast ratios, short response times, and excellent characteristics in terms of luminance, driving voltage, and response speed.

For example, an organic light-emitting device includes an anode, a cathode, and an organic layer located between the anode and the cathode, wherein the organic layer includes an emission layer. A hole transport region may be located between the anode and the emission layer, and an electron transport region may be located between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transition from an excited state to a ground state to thereby generate light, for example, visible light.

### SUMMARY

One or more embodiments include a composition and an organic light-emitting device including the same.

Additional aspects will be set forth in part in the description, which follows and, in part, will be apparent from the description, or may be learned by practice of the presented embodiments of the disclosure.

Provided is a composition including a first compound, a second compound and a third compound,

the first compound, the second compound and the third compound are different from each other,

the first compound satisfies one of Condition 1 and Condition 2, and

the second compound includes a compound represented by Formula 1

Condition 1

the first compound contains transition metal.

Condition 2

the difference between the triplet energy level of the first compound and the singlet energy level of the first compound is 0.4 eV or less, and the first compound emits delayed fluorescence

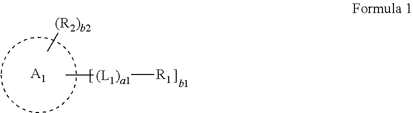

Ring A₁ in Formula 1 may be a condensed cyclic group in which 3 or more cyclic groups are condensed with each other, and the cyclic group may be a C₅-C₃₀ carbocyclic group or a C₁-C₃₀ heterocyclic group,

L₁ in Formula 1 may be a single bond, a C₅-C₃₀ carbocyclic group that is unsubstituted or substituted with at least one R₁₀ₐ, or a C₁-C₃₀ heterocyclic group that is unsubstituted or substituted with at least one R₁₀ₐ,

a1 in Formula 1 may be an integer from 1 to 5,

b1 in Formula 1 is an integer from 3 to 10, and

R₁ in Formula 1 may be a group represented by Formula 2 or Formula 3,

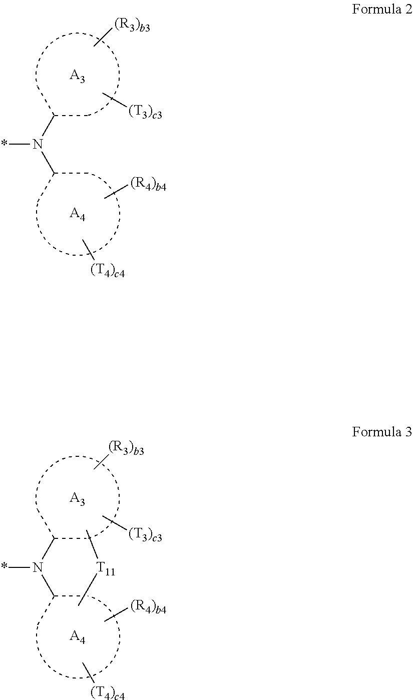

ring A₃ and ring A₄ in Formulae 2 and 3 may each independently be a C₅-C₃₀ carbocyclic group or a C₁-C₃₀ heterocyclic group,

T₃ and T₄ in Formulae 2 and 3 may each independently be a group represented by *—C(R₅)(R₆)(R₇) or *—Si(R₅)(R₆)(R₇),

c3 and c4 in Formulae 2 and 3 may each independently be an integer from 0 to 10, wherein, when c3 is 2 or more, two or more of T(s) may be identical to or different from each other, and when c4 is 2 or more, two or more of T₄(s) may be identical to or different from each other,

T₁₁ in Formula 3 may be a single bond, O, S, N(R₈), C(R₈)(R₉), or Si(R₈)(R₉),

R₂ to R₉ and R₁₀ₙ in Formulae 1 to 3 may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(QA)(Q₅), —B(Q₆)(Q₇), —P(═O)(Q₈)(Q₉), —P(═S)(Q₈)(Q₉), or —P(Q₈)(Q₉).

b2 to b4 in Formula 1 to 3 may each independently be an integer from 0 to 10, wherein, when b2 is 2 or more, two or more of R₂(s) may be identical to or different from each other, when b3 is 2 or more, two or more of R₃(s) may be identical to or different from each other, and when b4 is 2 or more, two or more of R₄(s) may be identical to or different from each other,

a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group,

a C₁-C₆₀ alkyl group, a C₂-C₆₀ alkenyl group, a C₂-C₆₀ alkynyl group, or a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂). —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(═S)(Q₈)(Q₉), —P(Q₁₈)(Q₁₉), or a combination thereof,

a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(═S)(Q₈)(Q₉), —P(Q₂₈)(Q₂₉), or a combination thereof,

—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), —P(═S)(Q₈)(Q₉), or —P(Q₃₈)(Q₃₉), or

a combination thereof,

wherein Q₁ to Q₉, Q₁₁ to Q₁₉, Q₂₁ to Q₂₉, and Q₃₁ to Q₃₉ are each independently hydrogen, deuterium: —F; —Cl; —Br; —I; a hydroxyl group: a cyano group: a nitro group: an amidino group: a hydrazine group: a hydrazone group: a carboxylic acid group or a salt thereof: a sulfonic acid group or a salt thereof: a phosphoric acid group or a salt thereof: a C₁-C₆₀ alkyl group which is unsubstituted or substituted with at least one of deuterium, a C₁-C₆₀ alkyl group, a C₆-C₆₀ aryl group, or a combination thereof: a C₂-C₆₀ alkenyl group: a C₂-C₆₀ alkynyl group: a C₁-C₆₀ alkoxy group: a C₁-C₆₀ alkylthio group; a C₃-C₁₀ cycloalkyl group: a C₁-C₁₀ heterocycloalkyl group: a C₃-C₁₀ cycloalkenyl group: a C₁-C₁₀ heterocycloalkenyl group: a C₆-C₆₀ aryl group which is unsubstituted or substituted with at least one of deuterium, a C₁-C₆₀ alkyl group, a C₆-C₆₀ aryl group, or a combination thereof: a C₆-C₆₀ aryloxy group: a C₆-C₆₀ arylthio group: a C₁-C₆₀ heteroaryl group: a C₂-C₆₀ alkyl heteroaryl group; a C₂-C₆₀ heteroaryl alkyl group; a C₁-C₆₀ heteroaryloxy group; a C₁-C₆₀ heteroarylthio group; a monovalent non-aromatic condensed polycyclic group: or a monovalent non-aromatic condensed heteropolycyclic group.

According to one or more aspects, an organic light-emitting device includes

a first electrode,

a second electrode, and

an organic layer located between the first electrode and the second electrode,

wherein the organic layer includes an emission layer, and

wherein the organic layer includes the composition.

According to one or more aspects, an organic light-emitting device includes:

a first electrode,

a second electrode,

m light-emitting units located between the first electrode and the second electrode and including at least one emission layer, and

m−1 charge generation layers located between neighboring two light-emitting units of the m light-emitting units and including an n-type charge generation layer and a p-type charge generation layer,

wherein m is an integer of 2 or more,

a maximum emission wavelength of light emitted from at least one light-emitting unit of the m light-emitting units may be different from a maximum emission wavelength of light emitted from at least one light-emitting unit of the remaining light-emitting units, and

the at least one emission layer includes the composition.

According to one or more aspects, an organic light-emitting device includes:

a first electrode,

a second electrode, and

m emission layers located between the first electrode and the second electrode,

wherein m is an integer of 2 or more,

a maximum emission wavelength of light emitted from at least one emission layer of the m emission layers may be different from a maximum emission wavelength of light emitted from at least one emission layer of the remaining emission layers, and

at least one emission layer includes the composition.

### BRIEF DESCRIPTION OF THE DRAWINGS

The above and other aspects, features, and advantages of certain embodiments of the disclosure will be more apparent from the following deception taken in conjunction with the accompanying drawings, in which:

FIG. 1 shows a schematic cross-sectional view of an organic light-emitting device according to one or more embodiments:

FIG. 2 is a schematic cross-sectional view of an organic light-emitting device according to another embodiment;

FIG. 3 is a schematic cross-sectional view of an organic light-emitting device according to another embodiment;

FIG. 4 is a graph of external quantum efficiency (EQE, %) versus luminance (candela per square meter, cd/m²) of the organic light-emitting device manufactured according to Example 1; and

FIG. 5 is a graph of luminance (%) versus time (hours, hr) of the organic light-emitting device manufactured according to Example 1;

FIG. 6 is a graph of EQE (%) versus luminance (cd/m²) of the organic light-emitting device manufactured according to Comparative Example 1:

FIG. 7 is a graph of luminance (%) versus time (hr) of the organic light-emitting device manufactured according to Comparative Example 1;

FIG. 8 is a graph of EQE (%) versus luminance (cd/m²) of the organic light-emitting device manufactured according to Example 2,

FIG. 9 is a graph of luminance (%) versus time (hr) of the organic light-emitting device manufactured according to Example 2:

FIG. 10 is a graph of EQE (%) versus luminance (cd/m²) of the organic light-emitting device manufactured according to Comparative Example 2:

FIG. 11 is a graph of luminance (%) versus time (hr) of the organic light-emitting device manufactured according to Comparative Example 2;

FIG. 12 is a graph of EQE (%) versus luminance (cd/m²) of the organic light-emitting device manufactured according to Example 3:

FIG. 13 is a graph of luminance (%) versus time (hr) of the organic light-emitting device manufactured according to Example 3;

FIG. 14 is a graph of EQE (%) versus luminance (cd/m²) of the organic light-emitting device manufactured according to Comparative Example 3;

FIG. 15 is a graph of luminance (%) versus time (hr) of the organic light-emitting device manufactured according to Comparative Example 3;

FIG. 16 is a graph of measured relative HOD values of Example 1-1, Comparative Example 1, and Comparative Example 1-1; and

FIG. 17 is a graph of measured relative HOD values of Example 2-1, Comparative Example 2, and Comparative Example 2-1.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

### Composition

The composition includes a first compound, a second compound, and a third compound. The first compound, the second compound, and the third compound are the same as described herein.

The first compound, the second compound, and the third compound included in the composition are different from each other. That is, the composition may include three or more compounds that are different from each other.

The composition may satisfy Condition 3 below:

E_{HOMO_C1}−0.4eV≤E_{HOMO_C2}≤E_{HOMO_C1}+0.1eV Condition 3

In Condition 3, E_{HOMO_C1} is the highest occupied molecular orbital (HOMO) energy level value of the first compound, E_{HOMO_C2} is the HOMO energy level value of the second compound, and eV is electron volts.

In this regard, the HOMO energy level value was measured using an atmospheric-pressure photoelectron spectroscopy device (manufactured by RIKEN KEIKI Co., Ltd.: AC3).

When the composition satisfies Condition 3, trapping of holes in the second compound is suppressed, and thus, direct formation of excitons due to electron-hole recombination in the second compound may be prevented. That is, it is possible to implement a mechanism in which excitons are first formed through a sensitizer (first compound), and then the excitons are moved to the second compound, which is the final luminous material, through energy transfer, and then light is emitted.

The composition may satisfy Condition 4 below:

R(HOD)_{H}/R(HOD)₀≤1.07 Condition 4

In Condition 4, R(HOD)_{H} is the relative HOD value of the composition including the first compound, the second compound, and the third compound, and R(HOD)₀ is the relative HOD value of the composition including the first compound and the third compound.

In this regard, the relative HOD value refers to the ratio of the applied voltage required to provide a specific current density to a single hole only device (HOD), and the relative HOD value is a value obtained by dividing the voltage applied to the HOD that is doped with the final luminous material (the second compound) by the voltage applied to the HOD that is not doped. The HOD structure may have, for example, the following structure, and the fabrication method therefor is the same as described herein:

ITO/HAT-CN (10 nm)/NPB (50 nm)/Test substance (40 nm)/NPB (10 nm)/Al

The first composition may satisfy one of Condition 1 or Condition 2:

Condition 1

The first compound contains transition metal

Condition 2

The difference between the triplet energy level of the first compound and the singlet energy level of the first compound is 0.4 eV or less, and the first compound may emit delayed fluorescence

According to one or more embodiments, the first compound may satisfy Condition 1.

For example, the first compound may include an organometallic compound represented by Formula 101:

M₁₁(L₁₁)ₙ₁₁(L₁₂)ₙ₁₂ Formula 101

wherein, in Formula 101.

M₁₁ may be a first-row transition metal of the Periodic Table of Elements, a second-row transition metal of the Periodic Table of Elements, or a third-row transition metal of the Periodic Table of Elements;

L₁₁ may be a ligand represented by one of Formulae 101-1 to 101-4;

L₁₂ may be a monodentate ligand or a bidentate ligand;

n11 may be 1,

n12 may be 0, 1, or 2:

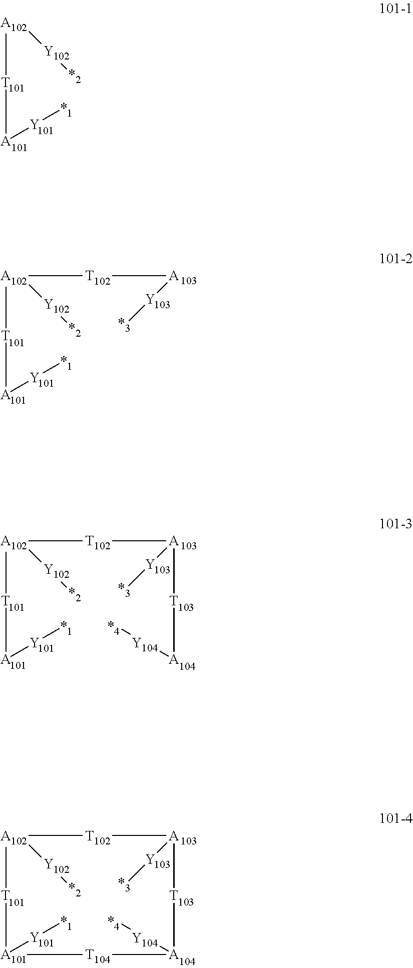

wherein, in Formulae 101-1 to 101-4,

A₁₀₁ to A₁₀₄ may each independently be a substituted or unsubstituted C₅-C₃₀ carbocyclic group, a substituted or unsubstituted C₁-C₃₀ heterocyclic group, or a non-cyclic group,

Y₁₀₁ to Y₁₀₄ may each independently be a chemical bond, O, S, N(R₉₁), B(R₉₁), P(R₉₁), or C(R₉₁)(R₉₂),

T₁₀₁ to T₁₀₄ may each independently be a single bond, a double bond, N(R93)*′, B(R₉₃), P(R₉₃), C(R₉₃)(R₉₄), Si(R₉₃)(R₉₄), Ge(R₉₃)(R₉₄), S. Se, O, C(═O), S(═O), S(═O)₂, —C(R₉₃)═, ═C(R₉₃)—, C(R₉₃)═C(R₉₄), C(═S), or C≡C,

a substituent of the substituted C₅-C₃₀ carbocyclic group, a substituent of the substituted C₁-C₃₀ heterocyclic group, and R₉₁ to R₉₄ may each independently be at least one of hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —C(═O)(Q₄₁), —S(═O)(Q₄₁), —S(═O)₂(Q₄₁), —N(Q₄₂)(Q₄₃). —B(Q₄₂)(Q₄₃), —Si(Q₄₄)(Q₄₅)(Q₄₆), —Ge(Q₄₄)(Q₄₅)(Q₄₆), —P(═O)(Q₄₇)(Q₄₈), —P(═S)(Q₄₇)(Q₄₈), and —P(Q₄₇)(Q₄₈), wherein each of a substituent of the substituted C₅-C₃₀ carbocyclic group and a substituent of substituted C₁-C₃₀ heterocyclic group may not be hydrogen,

*₁, *₂, *₃, and *₄ may each indicate a binding site to M₁₁, and

Q₄₁ to Q₄₈ may each independently be hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group that is substituted with at least one of deuterium, —F, a cyano group, a C₁-C₆₀ alkyl group, or a C₆-C₆₀ aryl group, and a C₆-C₆₀ aryl group that is substituted with deuterium, —F, a cyano group, a C₁-C₆₀ alkyl group, or a C₆-C₆₀ aryl group.

In one or more embodiments, the first compound may satisfy Condition 2.

For example, the first compound may include a thermally activated delayed fluorescence emitter represented by Formula 201 or 202 below:

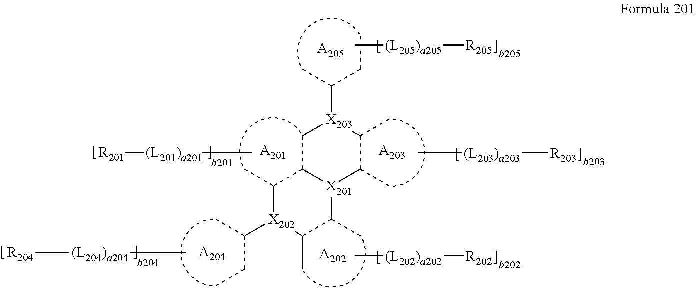

In Formula 201:

X₂₀₁ to X₂₀₃ may each independently be B or N,

A₂₀₁ to A₂₀₅ may each independently be a substituted or unsubstituted C₅-C₃₀ carbocyclic group or a substituted or unsubstituted C₁-C₃₀ heterocyclic group,

L₂₀₁ to L₂₀₅ may each independently be a single bond, a C₅-C₃₀ carbocyclic group that is unsubstituted or substituted with at least one R₂₀₀ₐ, or a C₁-C₃₀ heterocyclic group that is unsubstituted or substituted with at least one R₂₀₀ₐ,

a201 to a205 may each independently be an integer from 1 to 5.

R₂₀₁ to R₂₀₅, and R₂₀₀ₐ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₁-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), —P(═S)(Q₅₈)(Q₅₉), or —P(Q₅₈)(Q₅₉),

b201 to b205 may each independently be an integer from 0 to 10, wherein, when b201 is 2 or more, two or more of R₂₀₁(s) may be identical to or different from each other, when b202 is 2 or more, two or more of R₂₀₂(s) may be identical to or different from each other, when b203 is 2 or more, two or more of R₂₀₃(s) may be identical to or different from each other, when b204 is 2 or more, two or more of R₂₀₄(s) may be identical to or different from each other, and when b205 is 2 or more, two or more of R₂₀₅(s) may be identical to or different from each other,

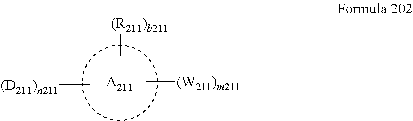

wherein, in Formula 202.

A₂₁₁ may be a substituted or unsubstituted C₅-C₃₀ carbocyclic group or a substituted or unsubstituted C₁-C₃₀ heterocyclic group,

W₂₁₁ may be an acceptor group,

D₂₁₁ may be a donor group,

m211 may be an integer from 1 to 4, and n211 may be an integer from 1 to 4;

R₂₁₁ may be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₂-C₆₀ aryloxy group, a substituted or unsubstituted C₂-C₆₀ arylthio group, a substituted or unsubstituted C₁₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), —P(═S)(Q₅₈)(Q₅₉), or —P(Q₅₈)(Q₅₉), and a plurality of R₂₁₁(s) may optionally be bonded to form a substituted or unsubstituted C₅-C₃₀ carbocyclic group or a substituted or unsubstituted C₁-C₃₀ heterocyclic group,

a substituent of the substituted C₅-C₃₀ carbocyclic group, substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group:

a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₁-C₆₀ arylthio group, a C₁-C₆₀ heteroaryl group, a monovalent non-aromatic condensed polycyclic group, a monovalent non-aromatic condensed heteropolycyclic group. —N(Q₆₁)(Q₆₂), —Si(Q₆₃)(Q₆₄)(Q₆₅), —Ge(Q₆₃)(Q₆₄)(Q₆₅), —B(Q₆₆)(Q₆₇), —P(═O)(Q₈₈)(Q₈₉), —P(═S)(Q₈₈)(Q₈₉), —P(Q₈₈)(Q₈₉), or a combination thereof:

a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₂-C₆₀ heteroarylthio group, a monovalent non-aromatic condensed polycyclic group, a monovalent non-aromatic condensed heteropolycyclic group, —N(Q₇₁)(Q₇₂), —Si(Q₇₃)(Q₇₄)(Q₇₅), —Ge(Q₇₃)(Q₇₄)(Q₇₅), —B(Q₇₆)(Q₇₇), —P(═O)(Q₇₈)(Q₇₉), —P(═S)(Q₇₈)(Q₇₉), —P(Q₇₈)(Q₇₉), or a combination thereof;

—N(Q₈₁)(Q₈₂), —Si(Q₈₃)(Q₈₄)(Q₈₅), —Ge(Q₈₃)(Q₈₄)(Q₈₅), —B(Q₈₆)(Q₈₇), —P(═O)(Q₈₈)(Q₈₉), —P(═S)(Q₈₈)(Q₈₉), or —P(Q₈₈)(Q₈₉); or

a combination thereof,

wherein Q₅₁ to Q₅₉, Q₅₁ to Q₅₉, Q₇₁ to Q₇₉ and Q₈₁ to Q₈₉ are each independently-hydrogen; deuterium; —F; —Cl; —Br; —I; a hydroxyl group: a cyano group; a nitro group; an amidino group, a hydrazine group; a hydrazone group, a carboxylic acid group or a salt thereof; a sulfonic acid group or a salt thereof; a phosphoric acid group or a salt thereof; a C₁-C₆₀ alkyl group which is unsubstituted or substituted with deuterium, a C₁-C₆₀ alkyl group, a C₆-C₆₀ aryl group, or a combination thereof, a C₂-C₆₀ alkenyl group; a C₂-C₆₀ alkynyl group; a C₁-C₆₀ alkoxy group; a C₃-C₆₀ alkylthio group, a C₃-C₁₀ cycloalkyl group; a C₁-C₁₀ heterocycloalkyl group; a C₃-C₁₀ cycloalkenyl group; a C₁-C₁₀ heterocycloalkenyl group, a C₆-C₆₀ aryl group which is unsubstituted or substituted with deuterium, a C₁-C₆₀ alkyl group, a C₆-C₆₀ aryl group, or a combination thereof; a C₆-C₆₀ aryloxy group; a C₆-C₆₀ arylthio group; a C₁-C₆₀ heteroaryl group; a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group; or a monovalent non-aromatic condensed heteropolycyclic group.

For example, W₂, may be a substituted or unsubstituted π electron-deficient nitrogen-free cyclic group, and

D₂₁₁ may be:

—F, a cyano group, or a π electron-deficient nitrogen-containing cyclic group;

a C₁-C₆₀ alkyl group, an π-electron deficient nitrogen-containing cyclic group, or an iT electron-deficient nitrogen-free cyclic group, each substituted with at least one of —F or a cyano group; or

an T-electron deficient nitrogen-containing cyclic group substituted with at least one of deuterium, a C₁-C₆₀ alkyl group, an π-electron deficient nitrogen-containing cyclic group, or an π electron-deficient nitrogen-free cyclic group,

wherein the π electron-deficient nitrogen-free cyclic group may be a phenyl group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, or a condensed cyclic group of two or more π electron-deficient nitrogen-free cyclic groups, and

the π electron-deficient nitrogen-containing cyclic group may be a cyclic group having at least one —N=moiety, and, for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnolne group, a phenanthrdine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, and a benzimidazolobenzimidazole group; or a condensed cyclic group in which two or more π electron-efficient nitrogen-containing cyclic groups are condensed with each other.

The first compound may be understood by referring to the description about a sensitizer as provided herein.

### Second Compound in Composition

The second compound includes a compound represented by Formula 1:

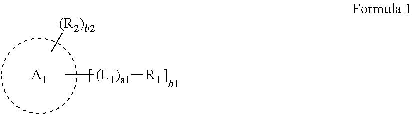

Ring A₁ in Formula 1 is a condensed cyclic group in which 3 or more cyclic groups are condensed with each other, and the cyclic group may be a C₅-C₃₀ carbocyclic group or a C₁-C₃₀ heterocyclic group.

In one or more embodiments, ring A₁ in Formula 1 may be an anthracene group, a phenalene group, a phenanthrene group, a tetracene group, a pyrene group, a chrysene group, a triphenylene group, a pentacene group, a perylene group, a fluoranthene group, a fluorene group, an acridine group, a phenanthridine group, a phenazine group, a phenoxazine group, a phenothiazine group, a xanthene group, a carbazole group, a dibenzofuran group, or a dibenzothiophene group.

For example, ring A₁ may be an anthracene group, a pyrene group, a chrysene group, or a perylene group.

L₁ in Formula 1 may be a single bond, a C₅-C₃₀ carbocyclic group that is unsubstituted or substituted with at least one R₁₀ₐ, or a C₁-C₃₀ heterocyclic group that is unsubstituted or substituted with at least one R₁₀ₐ.

In one or more embodiments, L₁ in Formula 1 may be a phenyl group, a heptalene group, an indene group, a naphthalene group, an azine group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, benzosilole group, a naphtho pyrrole group, a naphtho furan group, a naphtho thiophene group, a naphthosilole group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a benzosilolocarbazole group, a triindolobenzene group, an acridine group, a dihydroacridine group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, a benzoisoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, a benzonaphthofuran group, a benzonaphthothiophene group, an (indolo)phenanthrene group, a (benzofurano)phenanthrene group, or a (benzothieno)phenanthrene group, each unsubstituted or substituted with at least one R₁₀ₐ as provided herein.

a1 in Formula 1 may be an integer from 1 to 5, wherein, when a1 is 2 or more, two or more of L₁(s) may be identical to or different from each other.

R₁ in Formula 1 may be a group represented by Formula 2 or Formula 3:

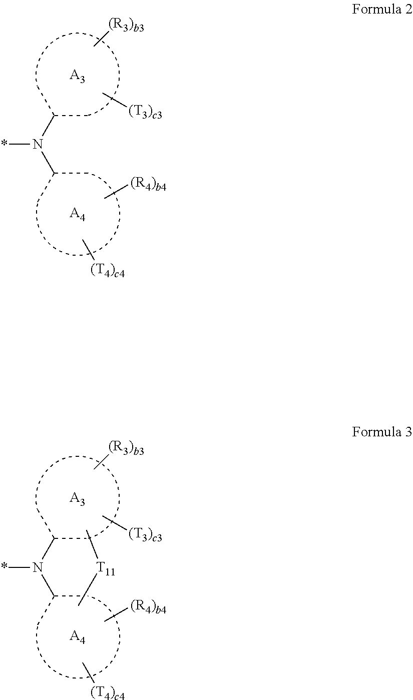

b1 in Formula 1 may be an integer from 3 to 10. For example, b1 may be an integer from 3 to 8. For example, b1 may be an integer from 3 to 6.

When b1 is 2 or more, two or more of R₁(s) may be identical to or different from each other.

Ring A₃ and ring A₄ in Formulae 2 and 3 may each independently be a C₅-C₃₀ carbocyclic group or a C₁-C₃₀ heterocyclic group.

In one or more embodiments, ring A₃ and ring A₄ may each independently be a phenyl group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a benzosilolocarbazole group, a triindolobenzene group, an acridine group, a dihydroacridine group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, a benzoisoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, a benzonaphthofuran group, a benzonaphthothiophene group, an (indolo)phenanthrene group, a (benzofurano)phenanthrene group, or a (benzothieno)phenanthrene group.

For example, ring A₃ and ring A₄ may each independently be a phenyl group, a naphthalene group, an anthracene group, a phenanthrene group, a triphenylene group, a fluorene group, dibenzosilole group, a carbazole group, a dibenzofuran group, or a dibenzothiophene group.

In Formulae 2 and 3, T₃ and T_{d} may each independently be a group represented by *—C(R₅)(R₆)(R₇) or *—Si(R₅)(R₆)(R₇), and c3 and c4 may each independently be an integer from 0 to 10, wherein, when c3 is 2 or greater, two or more T₃(s) may be identical to or different from each other, and when c4 is 2 or greater, two or more T₄(s) may be identical to each other or different from each other.

In one or more embodiments, the sum of c3 and c4 in Formulae 2 and 3 may be 1 or greater.

For example, the sum of c3 and c4 in Formulae 2 and 3 may be an integer from 1 to 4.

T₁₁ in Formula 3 may be a single bond, O, S, N(R₈), C(R₈)(R₉), or Si(R₈)(R₉). For example, T₁₁ may be a single bond.

R₂ to R₉ and R₁₀ₐ in Formulae 1 to 3 may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₂-C₁₀ cycloalkyl group, a substituted or unsubstituted C₁-C₁₀ heterocycloalkyl group, a substituted or unsubstituted C₂-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), —P(═S)(Q₈)(Q₉), or —P(Q₈)(Q₉).

b2 to b4 in Formula 1 to 3 may each independently be an integer from 0 to 10, wherein, when b2 is 2 or greater, two or more of R₂(s) may be identical to or different from each other, when b3 is 2 or greater, two or more of R(s) may be identical to or different from each other, and when b4 is 2 or greater, two or more of RA(S) may be identical to or different from each other.

In one or more embodiments, R₅ to R₇ in Formulae 2 and 3 may each independently be —CH₂, —CD₃, —CD₂H, —CDH₂, a phenyl group, or a group represented by one of Formulae 9-1 to 9-19:

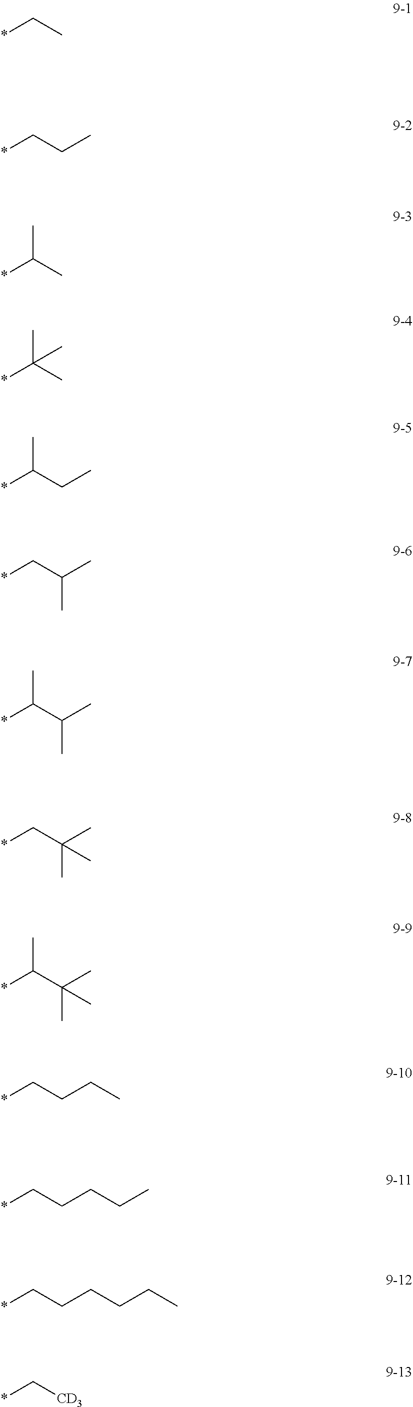

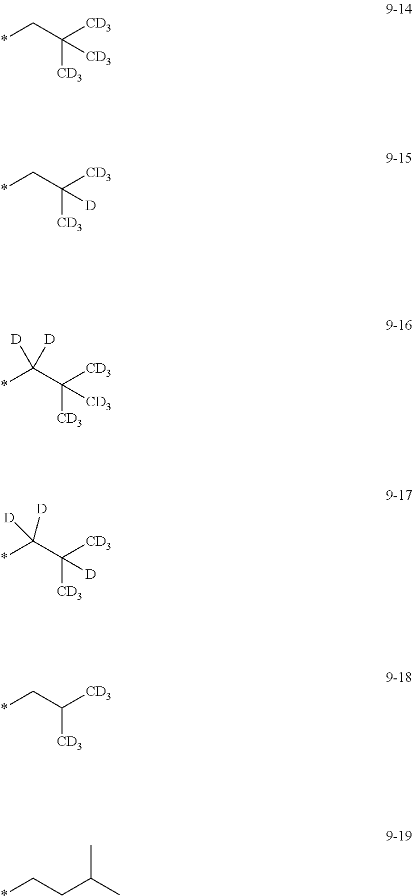

* in Formulae 9-1 to 9-19 indicates a binding site to a neighboring atom.

In one or more embodiments, the compound represented by Formula 1 may be represented by one of Formulae 1-1 to 1-4:

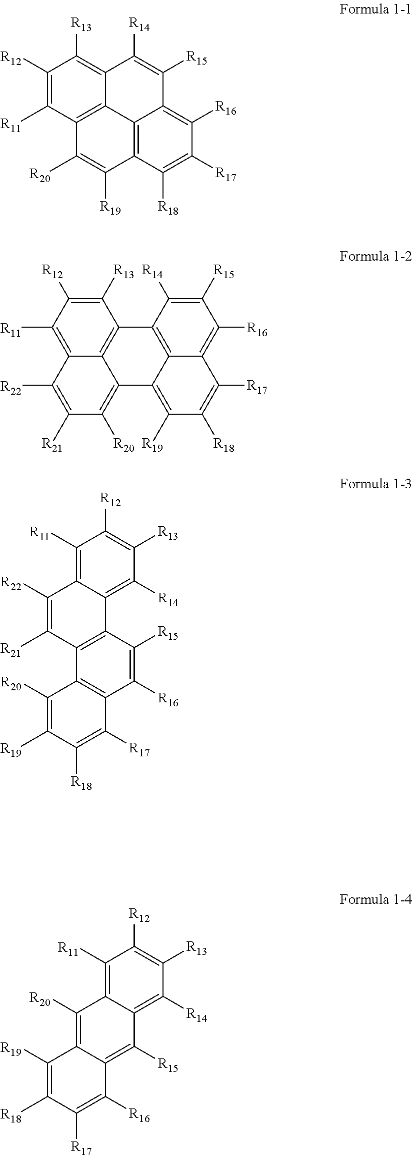

wherein, in Formulae 1-1 to 1-4,

R₁₁ to R₂₂ may each independently be a group represented by *-(L₁)ₐ₁-R₁, hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), —P(═S)(Q₈)(Q₈), or —P(Q₈)(Q₉),

at least three of R₁₁ to R₂₀ in Formulae 1-1 and 1-4 may each independently be a group represented by *-(L₁)ₐ₁-R₁,

at least three of R₁₁ to R₂₂ in Formulae 1-2 and 1-3 may each independently be a group represented by *-(L₁)ₐ₁-R₁, and

the substituents of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group are the same as described above.

For example,

i) three or more of R₁₁, R₁₃, R₁₆, and R₁₈ in Formula 1-1 may each be a group represented by *-(L₁)ₐ₁-R₁;

ii) three or more of R₁₂, R₁₅, R₁₆, and R₂₁ in Formula 1-2 may each be a group represented by *-(L₁)ₐ₁-R₁;

iii) three or more of R₁₃, R₁₆, R₁₉, and R₂₂ in Formula 1-3 may each be a group represented by *-(L₁)ₐ₁-R₁; and

iv) three or more of R₁₂, R₁₅, R₁₇, and R₂₀ in Formula 1-4 may each be a group represented by *-(L₁)ₐ₁-R₁.

In one or more embodiments, R₁ of Formula 1 may be a group represented by one of Formulae 2-1 to 2-42 and 3-1 to 3-36:

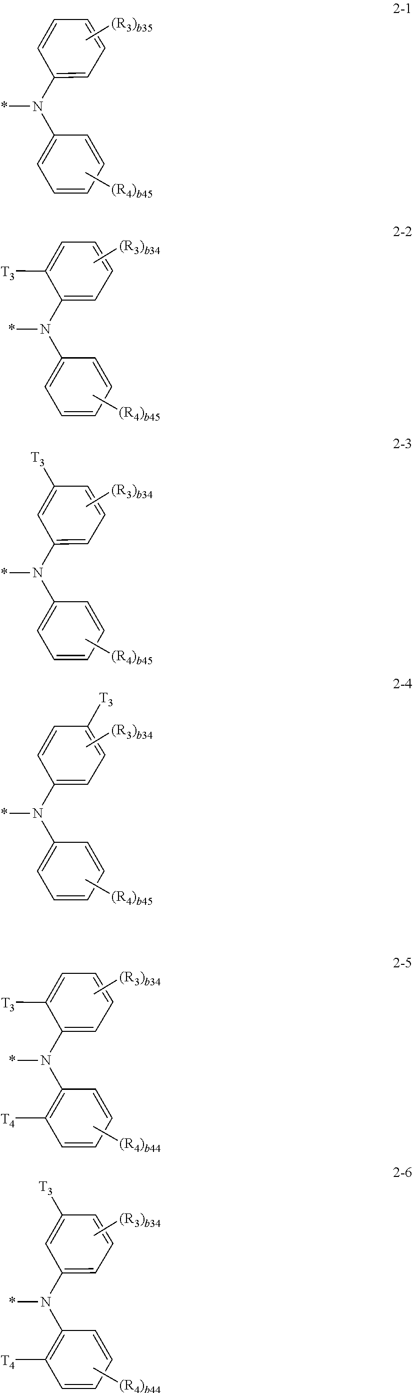

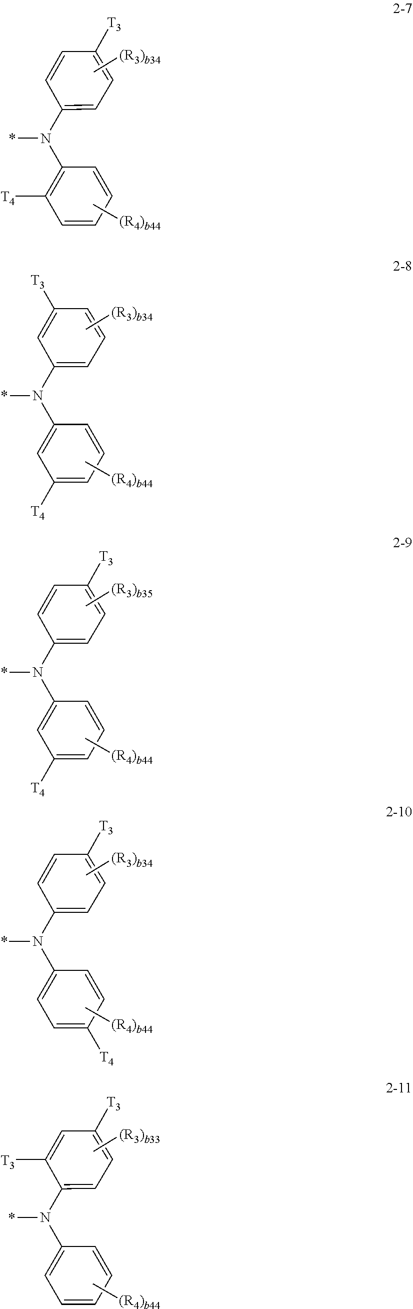

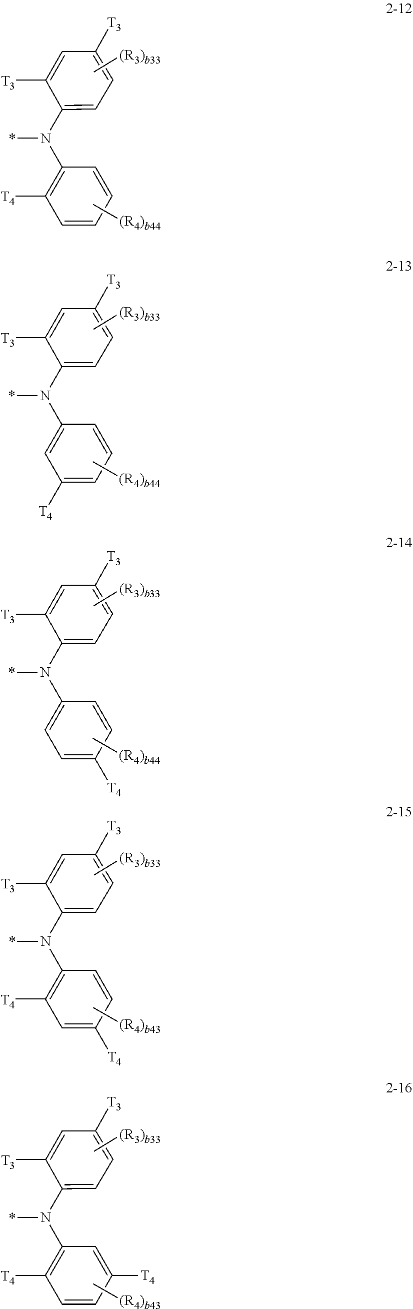

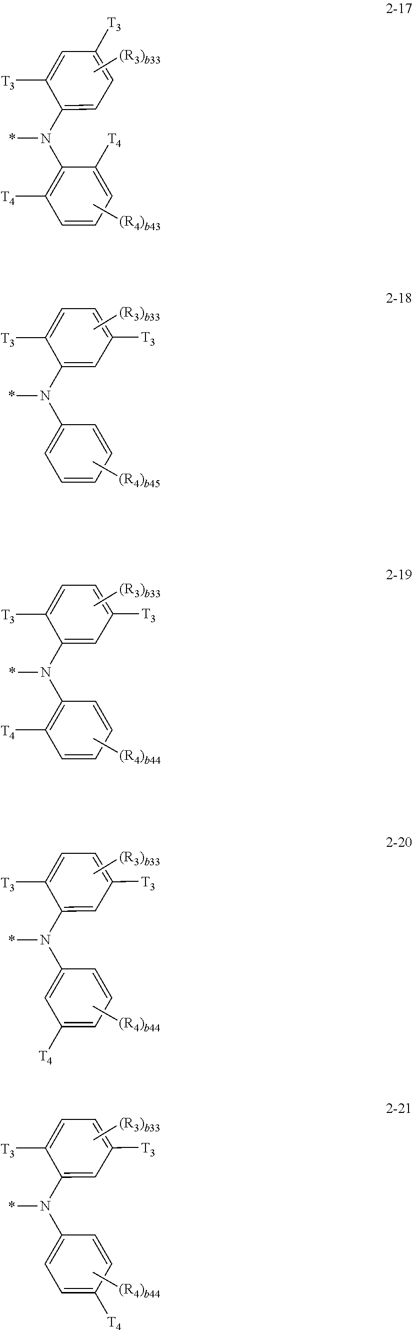

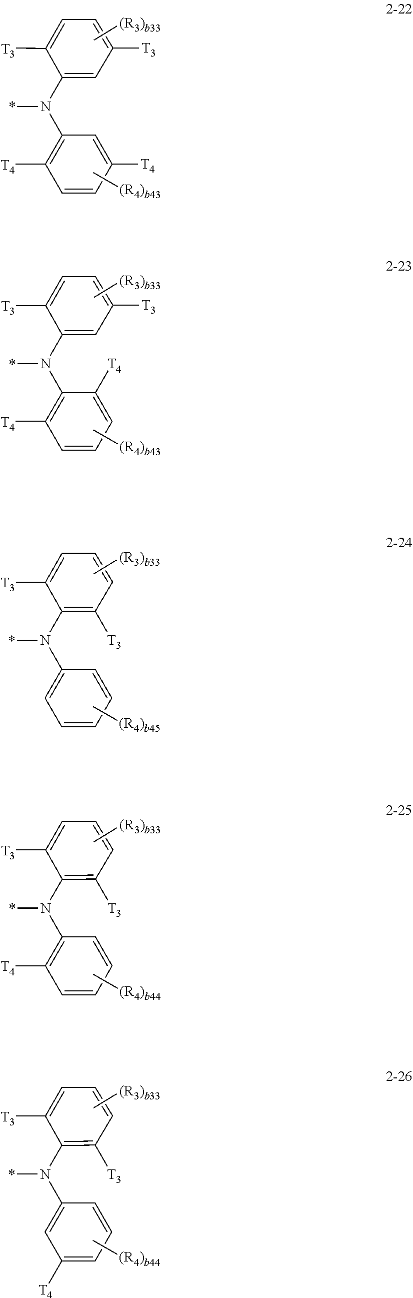

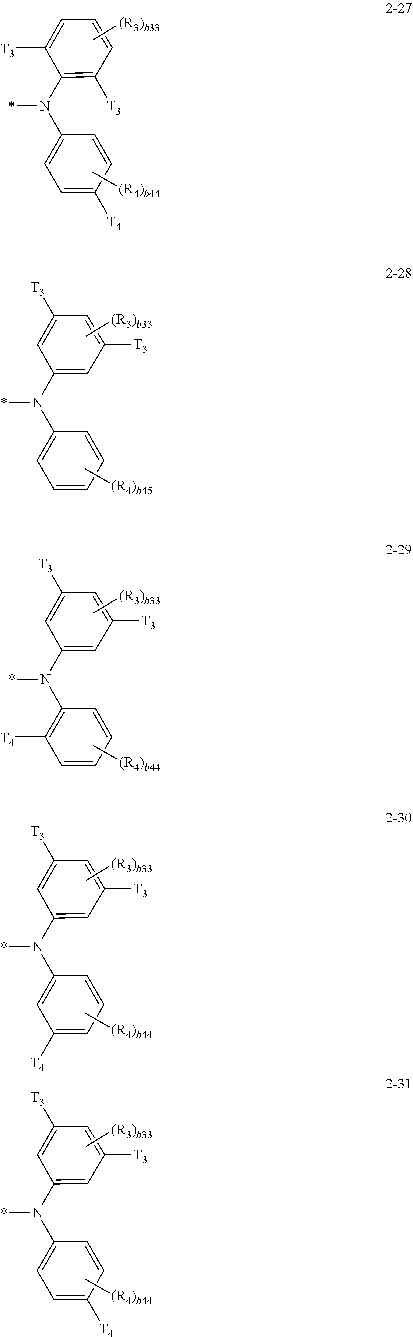

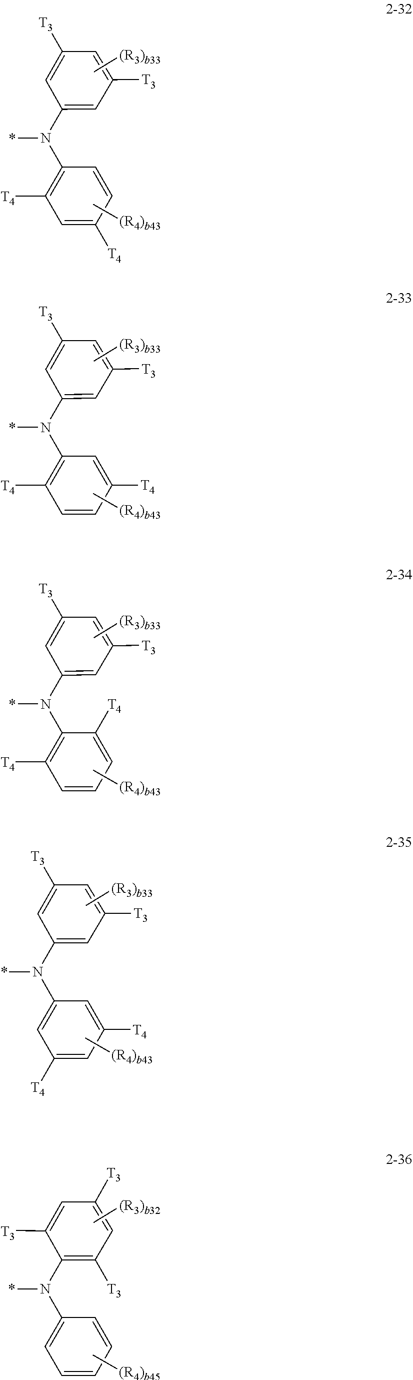

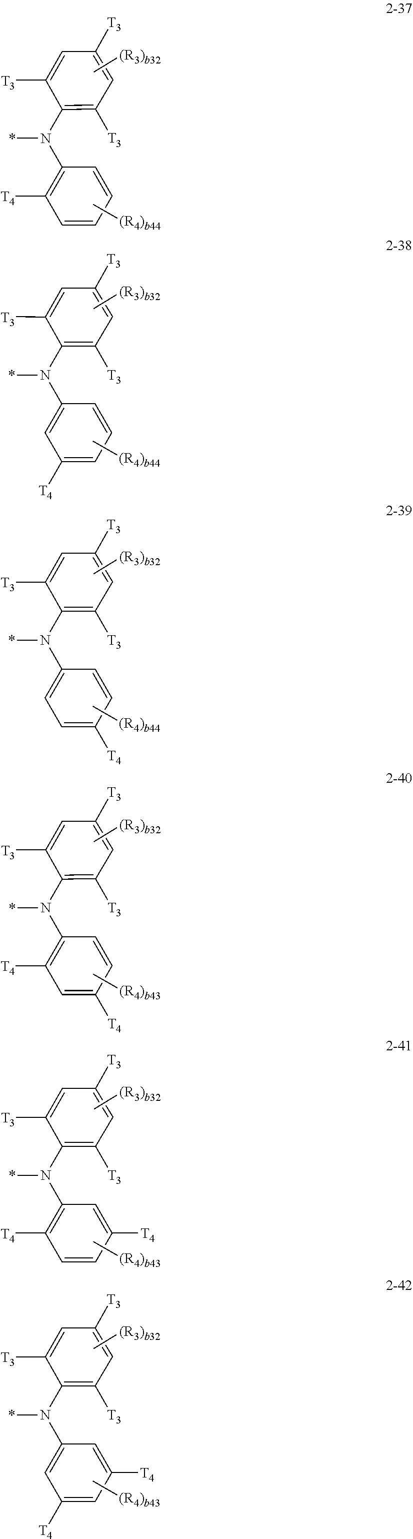

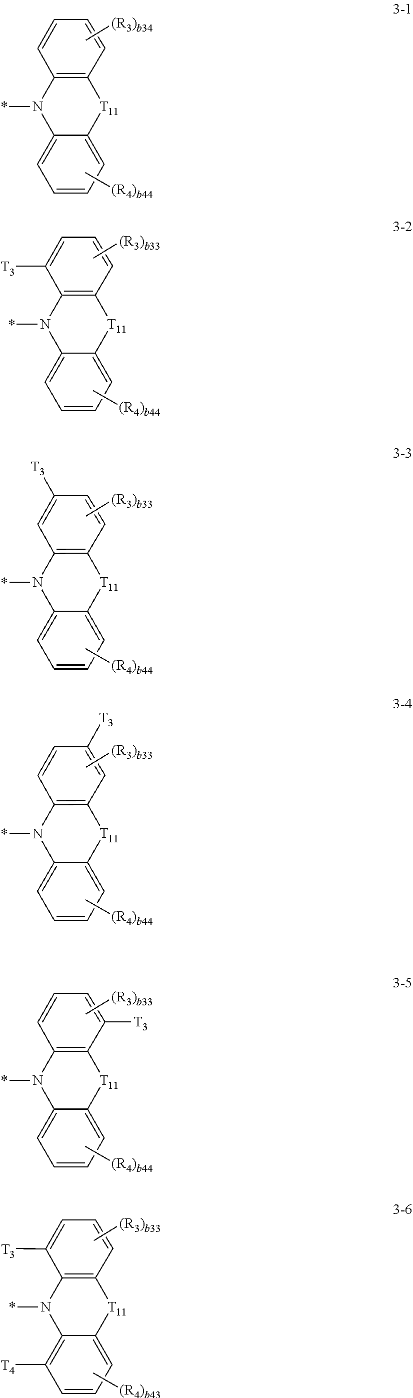

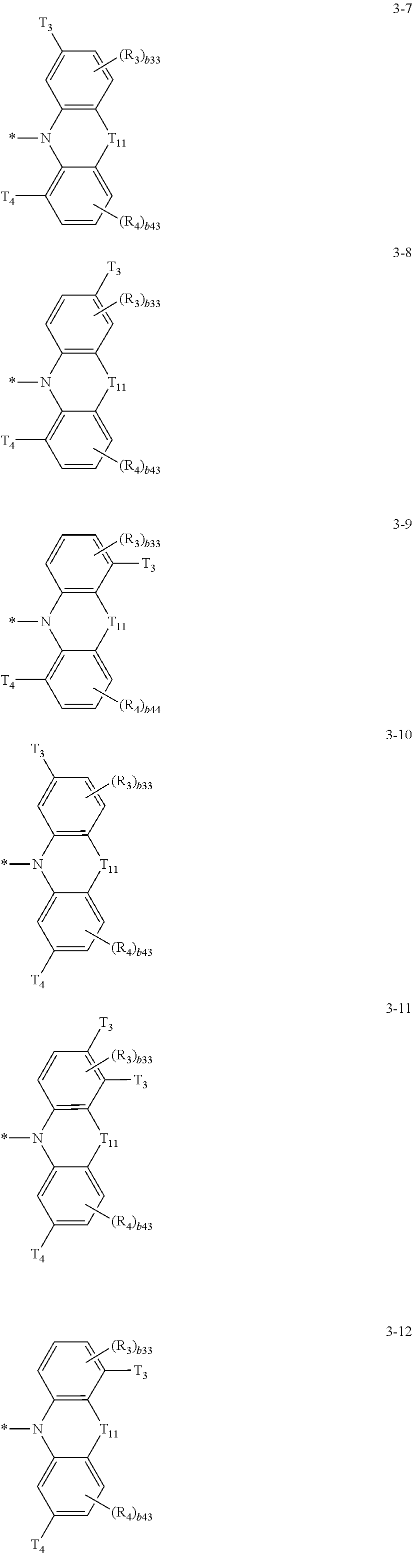

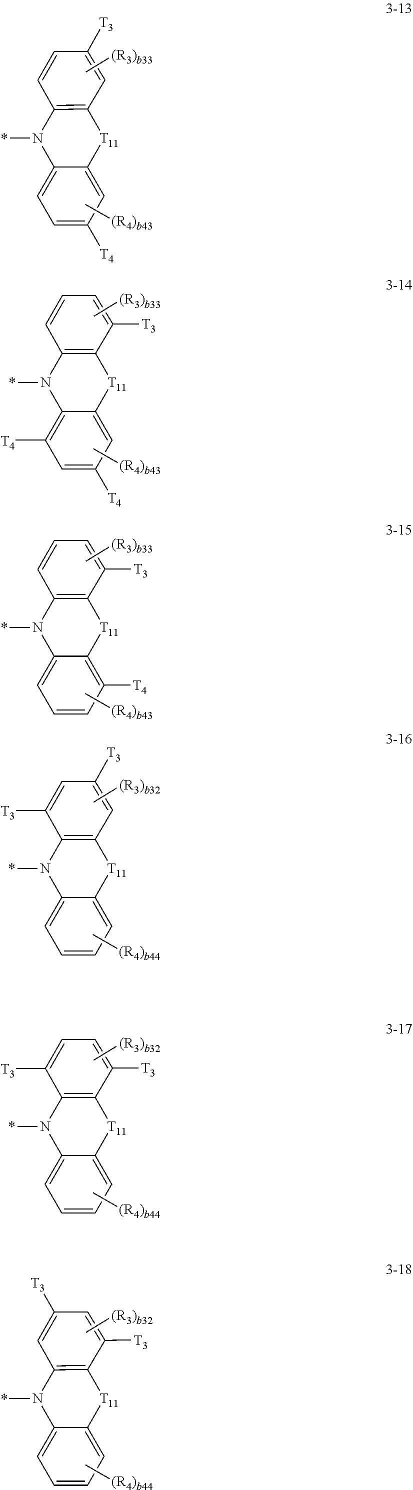

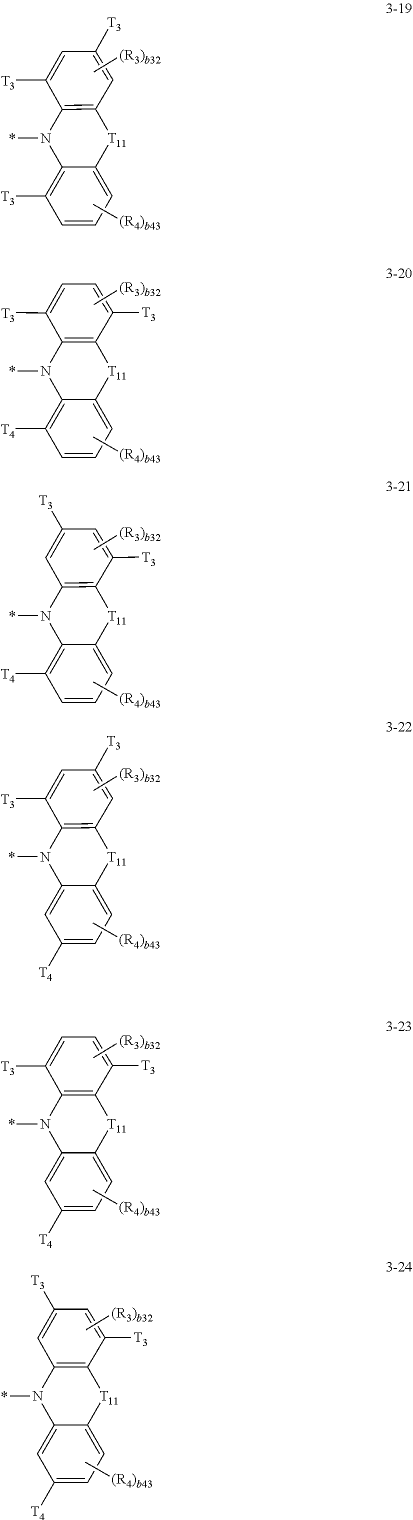

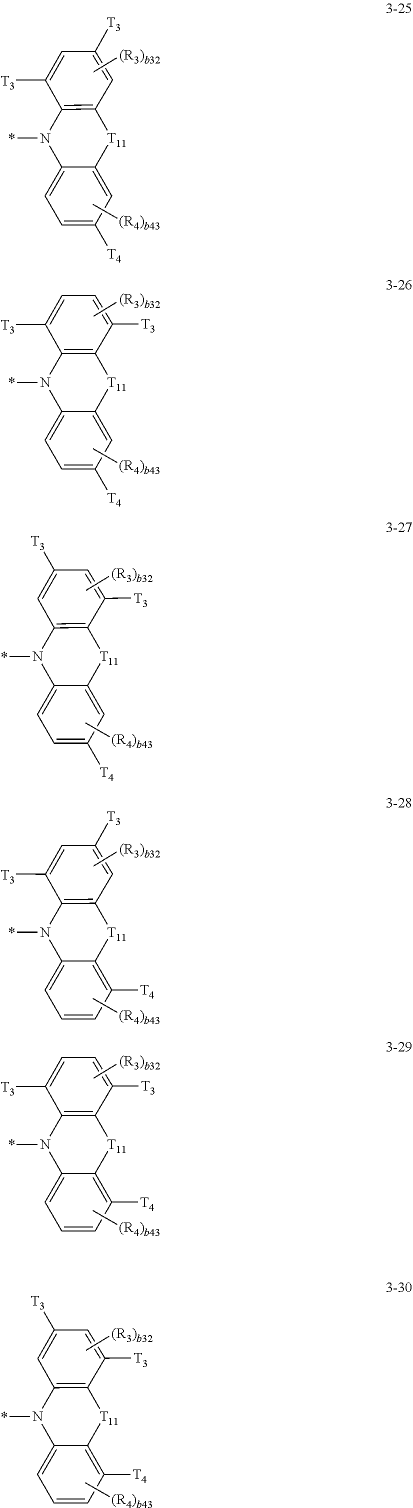

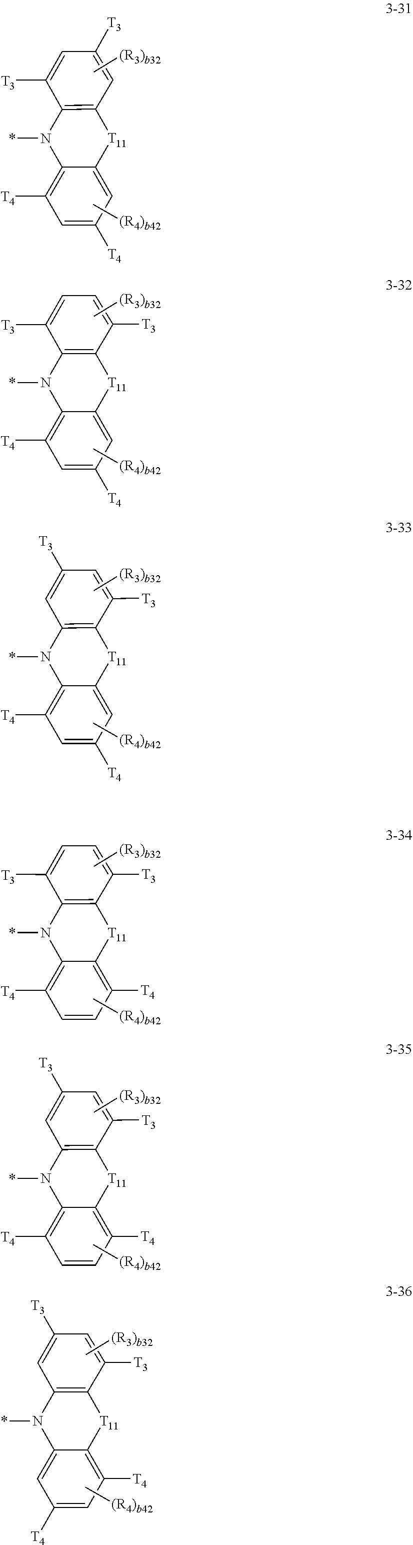

In Formulae 2-1 to 2-42 and 3-1 to 3-36,

T₃, T₄, T₁₁, R₃, and R₄ are the same as described above,

b32 and b42 may each independently be an integer from 0 to 2,

b33 and b43 may each independently be an integer from 0 to 3,

b34 and b44 may each independently be an integer from 0 to 4,

b35 and b45 may each independently be an integer from 0 to 5, and

* indicates a binding site to a neighboring atom.

In one or more embodiments, the second compound may be any one of Compounds FD1 to FD12:

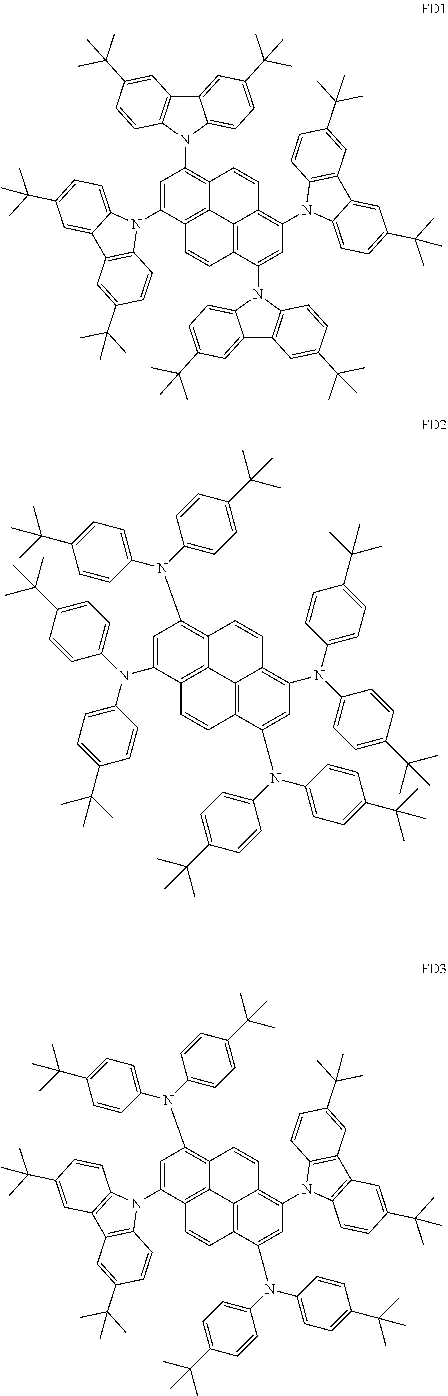

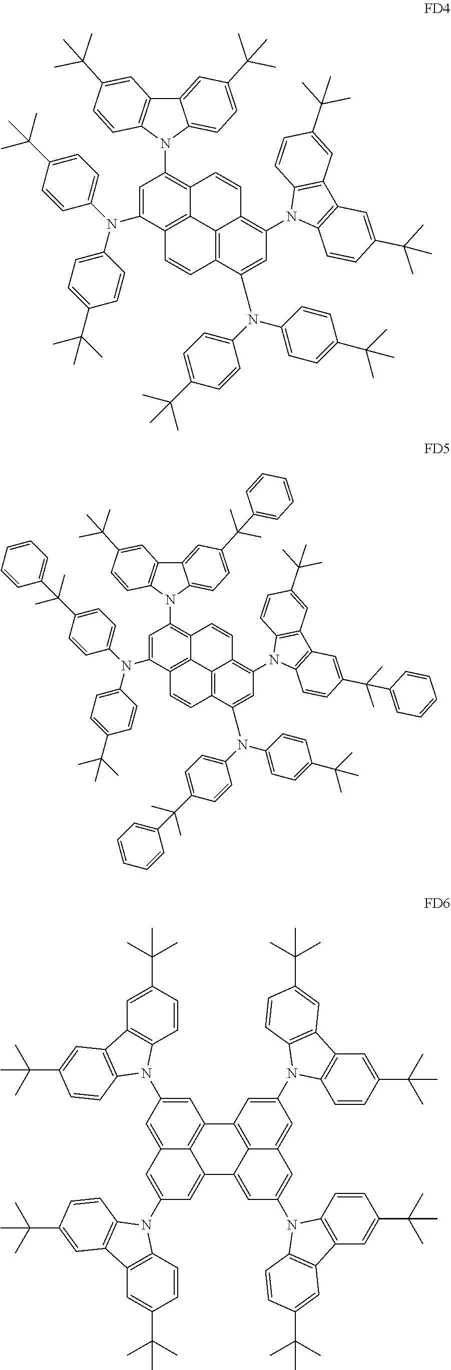

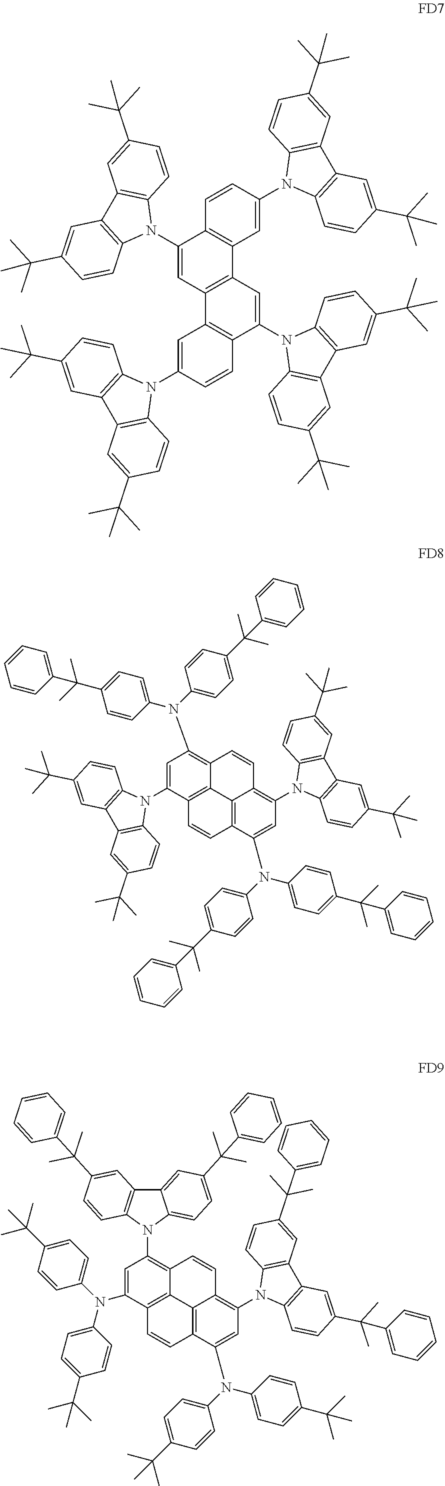

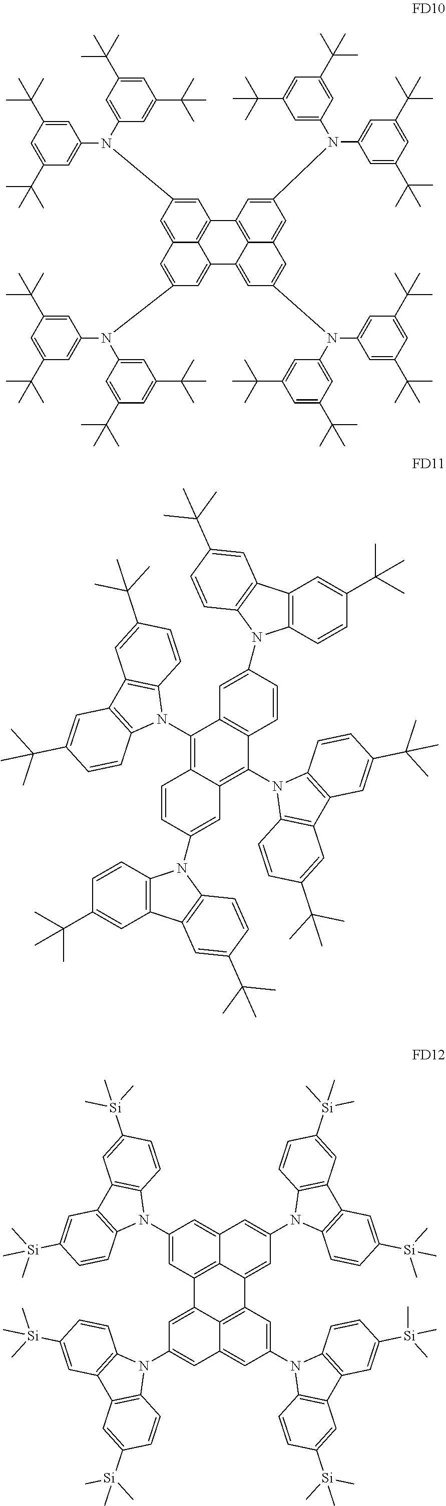

The second compound may emit blue light by having a condensed cyclic core in which three or more cyclic groups are condensed with each other.

Since the second compound includes three or more R₁ groups, a maximum spacing between a light-emitting portion of the sensitizer and a light-emitting portion of the dopant may be secured, suppressing dexter energy transfer.

Since the second compound may optionally contain one or more t-butyl substituents or trimethylsilyl (TMS) substituents, the maximum spacing between a light-emitting unit of the sensitizer and a light-emitting unit of the dopant may be secured, suppressing Dexter energy transfer.

The second compound may be understood by referring to the description about a dopant to be provided herein.

Third Compound in Composition

The third compound includes a bipolar compound, an electron-transporting compound, a hole-transporting compound, or a combination thereof,

the electron-transporting compound includes at least one electron-transporting moiety,

the hole-transporting compound may not include the electron-transporting moiety, and

the electron-transporting moiety may be a cyano group, a π electron-deficient nitrogen-containing cyclic group, or a group represented by one of the following

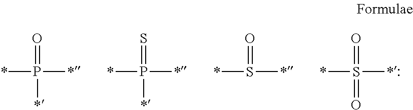

wherein *, *′, and *″ in the formulae above are each a binding site to a neighboring atom.

In one or more embodiments, the electron-transporting compound may include at least one π electron-deficient nitrogen-free cyclic group and at least one electron-transporting moiety,

the hole-transporting compound may include at least one π electron-deficient nitrogen-free cyclic group, and may not include an electron-transporting moiety, and

the electron-transporting moiety may be a cyano group or a iT electron-deficient nitrogen-containing cyclic group.

For example, in one or more embodiments, the electron-deficient nitrogen-containing cyclic group may be an imidazole group, a pyrazole group, a thiazole group, an isothiazole group, an oxazole group, an isoxazole group, a pyridine group, a pyrazine group, a pyridazine group, a pyrimidine group, indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, phenanthroline group, phenazine group, a benzimidazole group, an isobenzothiazole group, a benzoxazole group, a benzoisoxazole group, a triazole group, a tetrazole group, an oxadiazole group, a triazine group, thiadiazole group, an imidazopyridine group, an imidazopyrimidine group, and an azacarbazole group; or a condensed cyclic group of two or more π electron-deficient nitrogen-containing cyclic groups, and

the π electron-deficient nitrogen-free cyclic group may be a phenyl group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, or a condensed cyclic group of two or more π electron-deficient nitrogen-free cyclic groups.

For example, the electron-transporting compound includes

i) at least one of a cyano group, a pyrimidine group, a pyrazine group, and a triazine group and ii) a triphenylene group, and

the hole-transporting compound may include a carbazole group.

The third compound may be understood by referring to the description about a host as provided herein.

### Description of FIG. 1

FIG. 1 is a schematic view of an organic light-emitting device 10 according to one or more embodiments. Hereinafter, a structure and a manufacturing method of an organic light-emitting device according to an example of the present disclosure will be described with reference to FIG. 1.

The organic light-emitting device 10 of FIG. 1 includes: the first electrode 11; the second electrode 19; and an organic layer 10A located between the first electrode 11 and the second electrode 19 and including the emission layer 15, wherein the organic layer 10A includes the composition described above.

In one or more embodiments, the composition may be included in the emission layer 15.

In one or more embodiments, the emission layer 15 may include a sensitizer, a dopant, and a host, wherein the sensitizer may include the first compound of the composition, the dopant may include the second compound of the composition, and the host may include at least one third compound of the composition.

In one or more embodiments, the emission layer 15 may emit blue light.

In one or more embodiments, the organic layer 10A may include the hole transport region 12 located between the first electrode 11 and the emission layer 15, and the electron transport region 17 located between the emission layer 15 and the second electrode 19.

A substrate may be additionally located under the first electrode 11 or above the second electrode 19. For use as the substrate, any substrate that is used in organic light-emitting devices available in the art may be used, and the substrate may be a glass substrate or a transparent plastic substrate, each having excellent mechanical strength, thermal stability, transparency, surface smoothness, ease of handling, and water resistance

### First Electrode 11

In one or more embodiments, the first electrode 11 may be formed by depositing or sputtering a material for forming the first electrode 11 on the substrate. The first electrode 11 may be an anode. The material for forming the first electrode 11 may be selected from materials with a high work function to facilitate hole injection.

The first electrode 11 may be a reflective electrode, a semi-transmissive electrode, or a transmissive electrode. When the first electrode 11 is a transmissive electrode, a material for forming a first electrode may be indium tin oxide (ITO), indium zinc oxide (IZO), tin oxide (SnO₂), zinc oxide (ZnO), or a combination thereof, but embodiments of the present disclosure are not limited thereto. In one or more embodiments, when the first electrode 110 is a semi-transmissive electrode or a reflective electrode, a material for forming the first electrode 110 may be magnesium (Mg), silver (Ag), aluminum (Al), aluminum-lithium (Al—Li), calcium (Ca), magnesium-indium (Mg—In), magnesium-silver (Mg—Ag), or a combination thereof, but embodiments of the present disclosure are not limited thereto.

The first electrode 11 may have a single-layered structure or a multi-layered structure including two or more layers.

### Emission Layer 15

The emission layer 15 may include a host, a dopant, and a sensitizer.

The emission layer 15 may emit fluorescent light. That is, the dopant may be a material that may emit fluorescent light. The emission layer 15, which emits the fluorescent light, is clearly distinguished from an emission layer of the related art that emits phosphorescent light.

In general, it is known that since triplet excitons stay long in an excited state, they influence the decrease in the lifespan of organic light-emitting devices. However, according to the present disclosure, the dopant is used to reduce the time during which the triplet excitons of the sensitizer remains in the excited state. Accordingly, an organic light-emitting device including the dopant may have a prolonged lifespan.

In one or more embodiments, the greater the number of triplet excitons the sensitizer has, the more excess energy is accumulated in the sensitizer, resulting in an increased number of hot excitons. That is, the amount of triplet excitons of the sensitizer is proportional to the amount of hot excitons. The hot excitons break down various chemical bonds of a compound included in the emission layer 15 and/or a compound existing at the boundary of the emission layer 15 to decompose the compound Accordingly, the lifespan of organic light-emitting devices may be reduced. However, according to the present disclosure, by using dopants, the triplet excitons of the sensitizer may be quickly converted to singlet excitons of the dopant, ultimately reducing the amount of hot excitons and increasing the lifespan of an organic light-emitting device.

In this regard, “hot excitons” may be generated or increased by exciton-exciton annihilation due to an increase in the density of excitons in the emission layer 15, exciton-charge annihilation due to the charge imbalance in the emission layer 15, and/or radical ion pairs due to the delivery of electrons between a host and a dopant.

In addition, since the dopant emits fluorescent light, the formed organic light-emitting device may have high color purity. For example, as the singlet excitons in the excited state of the dopant at room temperature quickly switch to the ground state, the accumulation of singlet excitons in the state may be prevented and the lifespan of organic light-emitting devices may be improved.

In one or more embodiments, singlet and triplet excitons are formed at the host in the emission layer 15, and the singlet and triplet excitons formed in the host are transferred to the sensitizer and then to the dopant through Förster energy transfer (FRET). To obtain the high efficiency and long lifespan of the organic light-emitting device, hot excitons generated in the emission layer 15 may be controlled and to this end, optimization of energy transfer is required.

In one or more embodiments, when the sensitizer is a thermally activated delayed fluorescence (TADF) emitter satisfying the condition of ΔE_{ST}≤0.4 electron volts (eV), 25% of singlet excitons formed in the host is transferred to the sensitizer through FRET, and the energy of 75% of triplet excitons formed in the host is transferred to the singlet and the triplet of the sensitizer, and out of the energy, the energy transferred to the triplet is subjected to reverse intersystem crossing to a singlet, and then the singlet energy of the sensitizer is transferred to the dopant through FRET.

Furthermore, in one or more embodiments, when the sensitizer is an organic metallic compound containing at least one type of meta from a first-row transition metal of the Periodic Table of Elements, a second-row transition metal of the Periodic Table of Elements, or a third-row transition metal of the Periodic Table of Elements, about 75% of triplet excitons formed in the host is transferred to the sensitizer through Dexter energy transfer, and the energy of about 25% of singlet excitons formed in the host is transferred to the singlet and triplet of the sensitizer, and the energy transferred to the singlet is subjected to ISC to triplet, and then the triplet energy of the sensitizer is transferred to the dopant through FRET

Accordingly, by transferring all the singlet excitons and triplet excitons generated in the host of the emission layer 15 to the dopant, an organic light-emitting device having improved efficiency can be obtained. In addition, since an organic light-emitting device can be obtained with significantly reduced energy loss, the lifespan characteristics of the organic light-emitting device can be improved.

The amount of the sensitizer in the emission layer 15 may be from about 5 wt % to about 50 wt % Within these ranges, it is possible to achieve effective energy transfer in the emission layer 15, and accordingly, an organic light-emitting device having high efficiency and long lifespan can be obtained.

The emission layer 15 may consist of the host, the dopant, and the sensitizer. That is, the emission layer 15 may not further include materials other than the host, the dopant, and the sensitizer.

A thickness of the emission layer 15 may be in a range of about 100 Å to about 1,000 Å, for example, about 200 Å to about 600 Å. When the thickness of the emission layer 15 is within these ranges, excellent light-emission characteristics may be obtained without a substantial increase in driving voltage.

### Host in Emission Layer 15

The host may include at least one third compound.

The host may include no metal atoms or metal ions.

In one or more embodiments, the host may include one kind of host (i.e., a single host compound). When the host includes a single host, the single host may be a bipolar host, an electron-transporting host, or a hole-transporting host, as described herein.

In one or more embodiments, the host may include a mixture of two or more different hosts. For example, the host may be a mixture of an electron-transporting host and a hole-transporting host, a mixture of two types of electron-transporting hosts different from each other, or a mixture of two types of hole-transporting hosts different from each other. The electron-transporting host and the hole-transporting host may be understood by referring to the related description as provided herein.

In one or more embodiments, the host may include an electron-transporting host including at least one electron-transporting moiety and a hole-transporting host that is free of an electron-transporting moiety.

The electron-transporting moiety used herein may be a cyano group, a π electron-deficient nitrogen-containing cyclic group, or a group represented by one of the following Formulae.

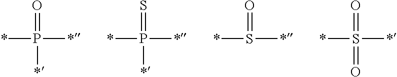

In the formulae, *, *′, and *″ are each binding sites to neighboring atoms.

In one or more embodiments, the electron-transporting host of the emission layer 15 may include at least one of a cyano group and a π electron-deficient nitrogen-containing cyclic group.

In one or more embodiments, the electron-transporting host in the emission layer 15 may include at least one cyano group.

In one or more embodiments, the electron-transporting host in the emission layer 15 may include at least one cyano group and at least one π electron deficient nitrogen-containing cyclic group.

In one or more embodiments, the host may include an electron-transporting host and a hole-transporting host, wherein the electron-transporting host may include at least one π electron-deficient nitrogen-free cyclic group and at least one electron-transporting moiety, and the hole-transporting host may include at least one π electron-deficient nitrogen-free cyclic group and may not include an electron-transporting moiety.

In one or more embodiments, the electron-transporting host may be a compound represented by Formula E-1, and

the hole-transporting host may be a compound represented by Formula H-1, but embodiments of the present disclosure are not limited thereto:

[Ar₃₀₁]_{xb11}−[(L₃₀₁)_{xb1}−R₃₀₁]_{xb21} Formula E-1

wherein, in Formula E-1,

Ar₃₀₁ may be a substituted or unsubstituted C₅-C₆₀ carbocyclic group or a substituted or unsubstituted C₁-C₆₀ heterocyclic group,

xb11 may be 1, 2, or 3,

L₃₀₁ may each independently be a single bond, a group represented by one of the following formulae, a substituted or unsubstituted C₅-C₆₀ carbocyclic group, or a substituted or unsubstituted C₁-C₆₀ heterocyclic group, and *, *′ and *″ in the following formulae are each a binding site to a neighboring atom,

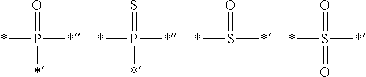

xb1 may be an integer from 1 to 5,

R₃₀₁ may be hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂). —C(═O)(Q₃₀₁), —S(═O)₂(Q₃₀₁), —S(═O)(Q₃₀₁), —P(═O)(Q₃₀₁)(Q₃₀₂), —P(═S)(Q₃₀₁)(Q₃₀₂), or P(Q₃₀₁)(Q₃₀₂),

xb21 may be an integer from 1 to 5,

Q₃₀₁ to Q₃₀₃ may each independently be a C₁-C₁₀ alkyl group, a C₁-C₁₀ alkoxy group, a phenyl group, a biphenyl group, a terphenyl group, or a naphthyl group, and

at least one of Condition A to Condition C may be satisfied:

Condition A

Ar₃₀₁, L₃₀₁, and R₃₀₁ in Formula E-1 may each independently include a π electron-deficient nitrogen-containing cyclic group

Condition B

L₃₀₁ in Formula E-1 may be a group represented by one of the following groups:

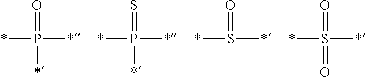

Condition C

R₃₀₁ in Formula E-1 may be a cyano group, —S(═O)₂(Q₃₀₁), —S(═O)(Q₃₀₁), —P(═O)(Q₃₀₁)(Q₃₀₂), or —P(═S)(Q₃₀₁)(Q₃₀₂)

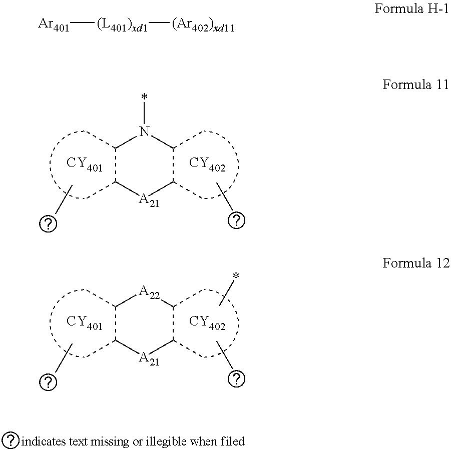

wherein, in Formulae H-1, 11, and 12,

L₄₀₁ may be:

a single bond; or

a phenyl group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with at least one of deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a quaterphenyl group, or —Si(Q₄₀₁)(Q₄₀₂)(Q₄₀₃),

xd1 may be an integer from 1 to 10, wherein when xd1 is 2 or more, two or more of L₄₀₁(s) may be identical to or different from each other,

Ar₄₀₁ may be a group represented by one of Formulae 11 and 12,

Ar₄₀₂ may be a group represented by one of Formula 11 and 12, a phenyl group, a naphthyl group, a fluorenyl group, a carbazolyl group, a dibenzofuranyl group, a dibenzothiophenyl group, a biphenyl group, a terphenyl group, or a triphenylenyl group; or

a phenyl group, a naphthyl group, a fluorenyl group, a carbazolyl group, a dibenzofuranyl group, a dibenzothiophenyl group, a biphenyl group, a terphenyl group, and a triphenylenyl group, each substituted with at least one of deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, a terphenyl group, or a triphenylenyl group,

CY₄₀₁ and CY₄₀₂ may each independently be a phenyl group, a naphthalene group, a fluorene group, a carbazole group, a benzocarbazole group, an indolocarbazole group, a dibenzofuran group, a dibenzothiophene group, a dibenzosilole group, a benzonaphthofuran group, a benzonaphthothiophene group, or a benzonaphthosilole group,

A₂₁ may be a single bond, O, S, N(R₅₁), C(R₅₁)(R₅₂), or Si(R₅₁)(R₅₂),

A₂₂ may be a single bond, O, S, N(R₅₃), C(R₅₃)(R₅₄), or Si(R₅₃)(R₅₄),

at least one of A₂₁ and A₂₂ in Formula 12 is not a single bond,

R₅₁ to R₅₄, R₆₀, and R₇₀ may each independently be:

hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group:

a C₁-C₂₀ alkyl group or a C₁-C₂₀ alkoxy group, each substituted with at least one of deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, or a dibenzothiophenyl group;

a π electron-deficient nitrogen-free cyclic group (for example, a phenyl group, a naphthyl group, a fluorenyl group, a carbazolyl group, a dibenzofuranyl group, a dibenzothiophenyl group, a biphenyl group, a terphenyl group, or a triphenylenyl group):

a π electron-deficient nitrogen-free cyclic group (for example, a phenyl group, a naphthyl group, a fluorenyl group, a carbazolyl group, a dibenzofuranyl group, a dibenzothiophenyl group, a biphenyl group, a terphenyl group, and a triphenylenyl group), each substituted with at least one of deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, or a biphenyl group; or

—Si(Q₄₀₄)(Q₄₀₅)(Q₄₀₆),

e1 and e2 may each independently be an integer from 0 to 10,

Q₄₀₁ to Q₄₀₆ may each independently be hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, a biphenyl group, a terphenyl group, or a triphenylenyl group, and

* indicates a binding site to an adjacent atom.

In one or more embodiments, Ar₃₀₁ and L₃₀₁ in Formula E-1 may each independently be a phenyl group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one of deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(Q₃₁)(Q₃₂), —P(═O)(Q₃₁)(Q₃₂), or —P(═S)(Q₃₁)(Q₃₂),

at least one of L₃₀₁(s) in the number of xb1 may each independently be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyndine group, an imidazopyrimidine group, or an azacarbazole group, each unsubstituted or substituted with at least one of deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

R₃₀₁ may be hydrogen, deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a quaterphenyl group, a naphthyl group, a cyano-containing phenyl group, a cyano-containing biphenyl group, a cyano-containing terphenyl group, a cyano-containing quaterphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃): —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₃₁ to Q₃₃ may each independently be a C₁-C₁₀ alkyl group, a C₁-C₁₀ alkoxy group, a phenyl group, a biphenyl group, a terphenyl group, or a naphthyl group, but embodiments of the present disclosure are not limited thereto.

In one or more embodiments,

Ar₃₀₁ may be a phenyl group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, or a dibenzothiophene group, each unsubstituted or substituted with at least one of deuterium. —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂); or

a group represented by one Formulae 5-1 to 5-3 or Formulae 6-1 to 6-33, and

L₃₀₁ may be a group represented by one of Formulae 5-1 to 5-3 and Formulae 6-1 to 6-33:

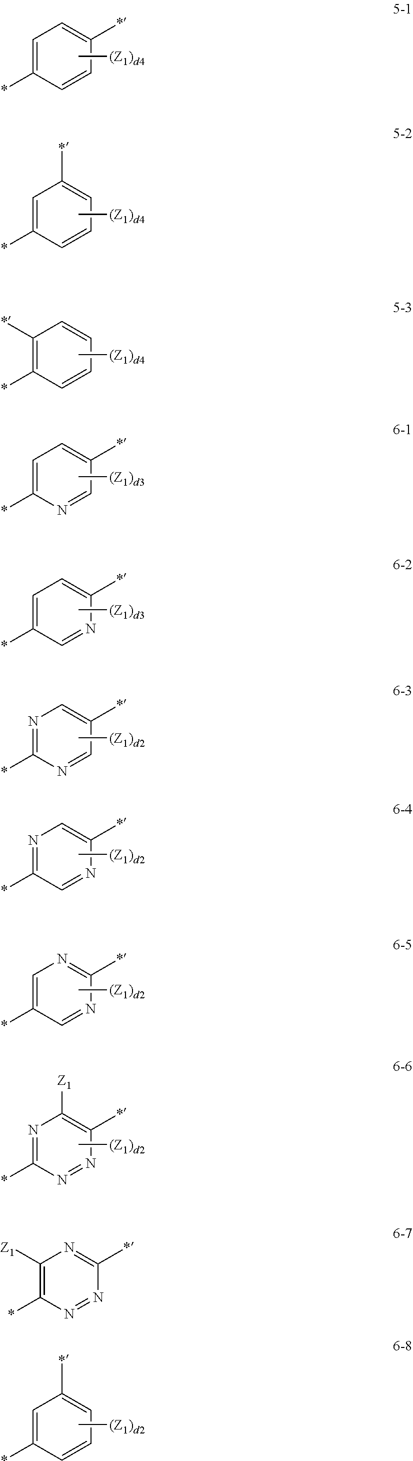

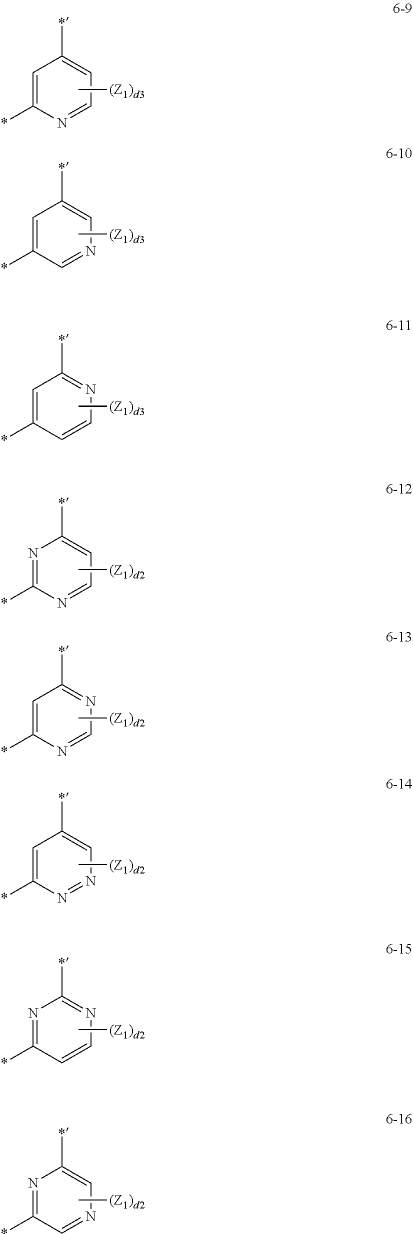

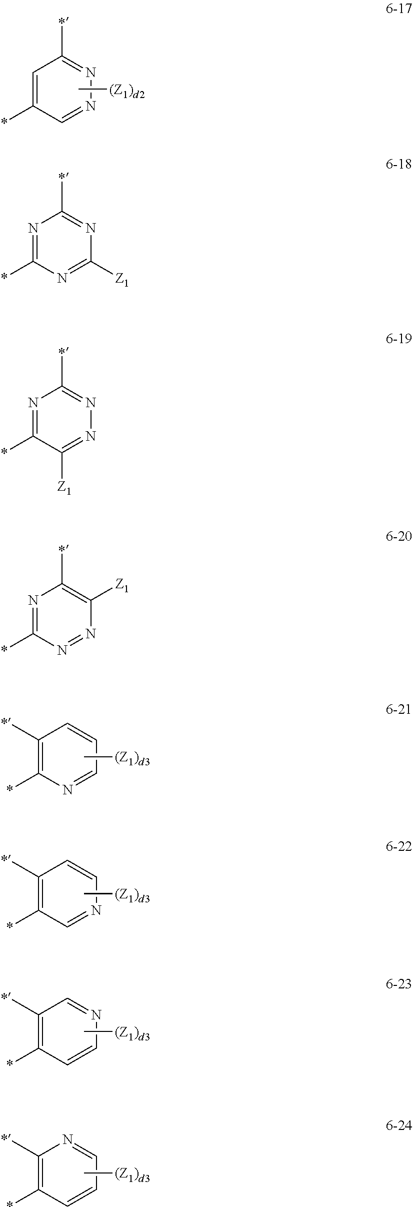

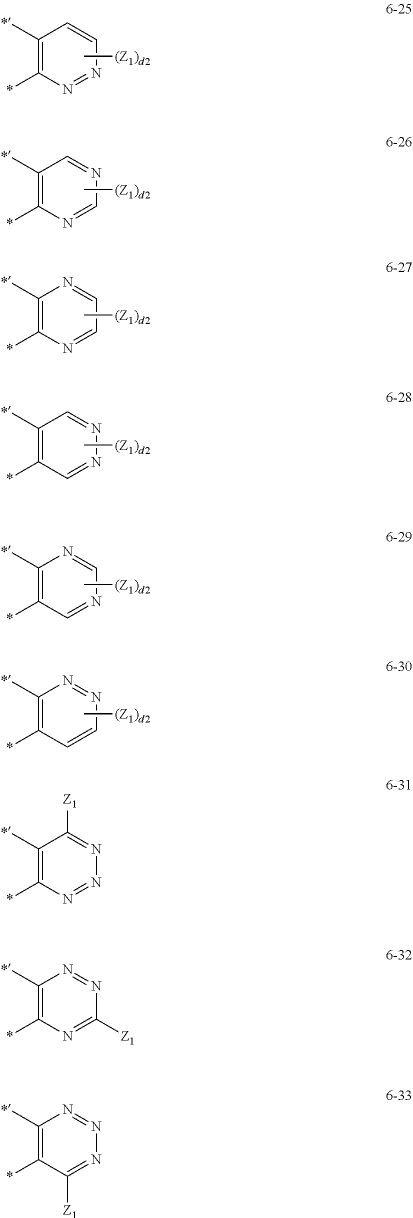

wherein, in Formulae 5-1 to 5-3 and 6-1 to 6-33,

Z₁ may be hydrogen, deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyrindinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

d4 may be 0, 1, 2, 3, or 4,

d3 may be 0, 1, 2, or 3,

d2 may be 0, 1, or 2, and

* and *′ each indicate a binding site to a neighboring atom,

wherein Q₃₁ to Q₃₃ are the same as described above.

In one or more embodiments, L₃₀₁ may be a group represented by one of Formulae 5-2, 5-3, or 6-8 to 6-33.

In one or more embodiments, R₃₀₁ may be a cyano group or a group represented by one of Formulae 7-1 to 7-18, and at least one of Ar₄₀₂(s) in the number of xd11 may be a group represented by one of Formulae 7-1 to 7-18, but embodiments of the present disclosure are not limited thereto:

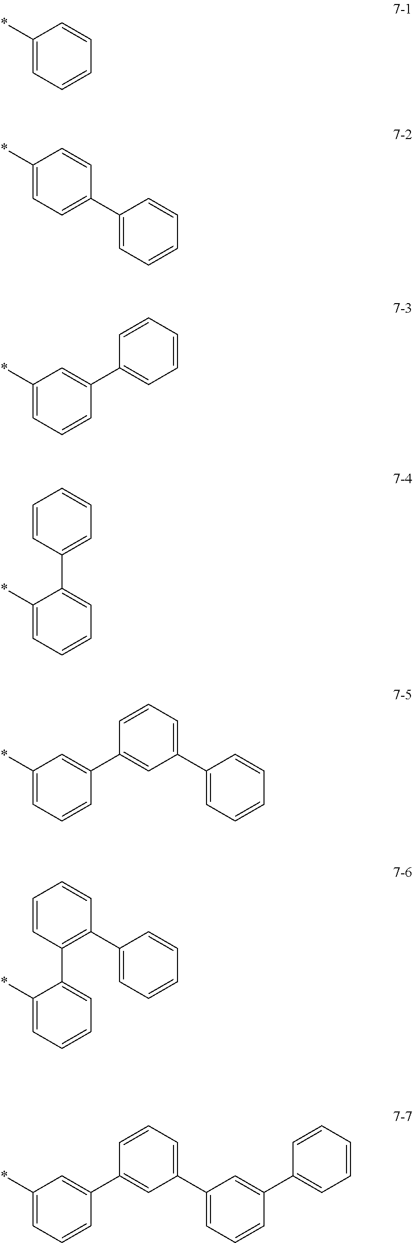

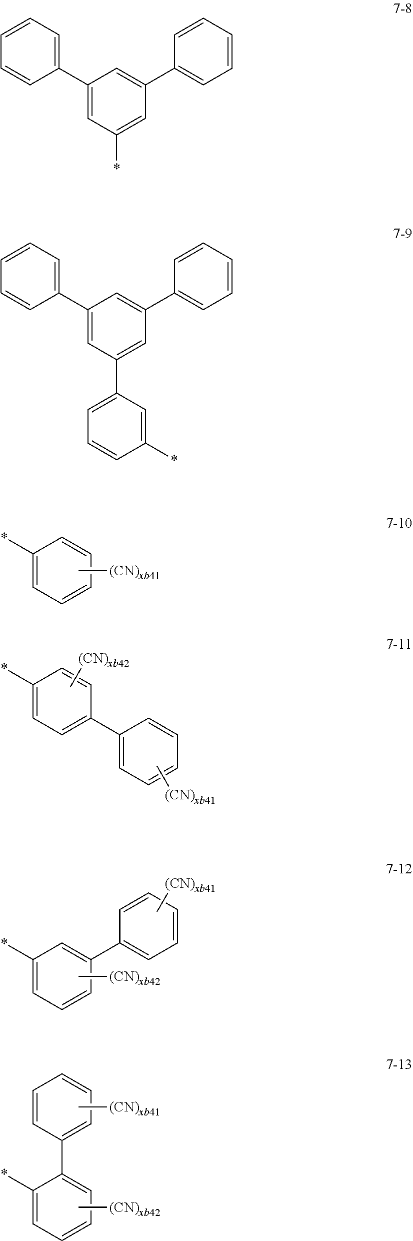

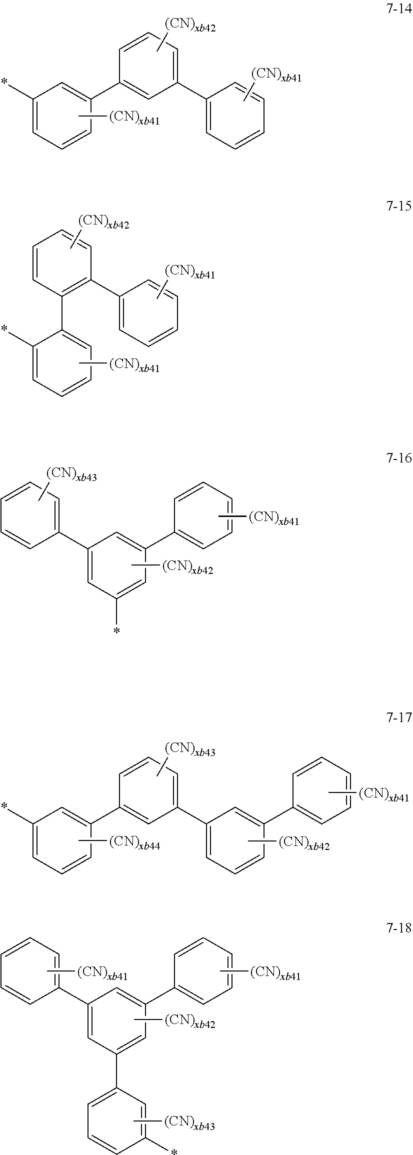

wherein, in Formulae 7-1 to 7-18,

xb41 to xb44 may each be 0, 1, or 2, wherein xb41 in Formula 7-10 is not 0, the sum of xb41 and xb42 in Formulae 7-11 to 7-13 is not 0, the sum of xb41, xb42, and xb43 in Formulae 7-14 to 7-16 is not 0, the sum of xb41, xb42, xb43, and xb44 in Formulae 7-17 and 7-18 is not 0, and *indicates a binding site to a neighboring atom.

Two or more Ar₃₀₁(s) in Formula E-1 may be identical to or different from each other, two or more of L₃₀₁(s) may be identical to or different from each other, two or more of L₄₀₁(s) in Formula H-1 may be identical to or different from each other, and two or more of Ar₄₀₂(s) in Formula H-1 may be identical to or different from each other.

In one or more embodiments, the electron-transporting host includes i) at least one of a cyano group, a pyrimidine group, a pyrazine group, and a triazine group and ii) a triphenylene group, and the hole-transporting host may include a carbazole group.

In one or more embodiments, the electron-transporting host may include at least one cyano group.

The electron-transporting host may be, for example, a compound from groups HE1 to HE7, but embodiments of the present disclosure are not limited thereto:

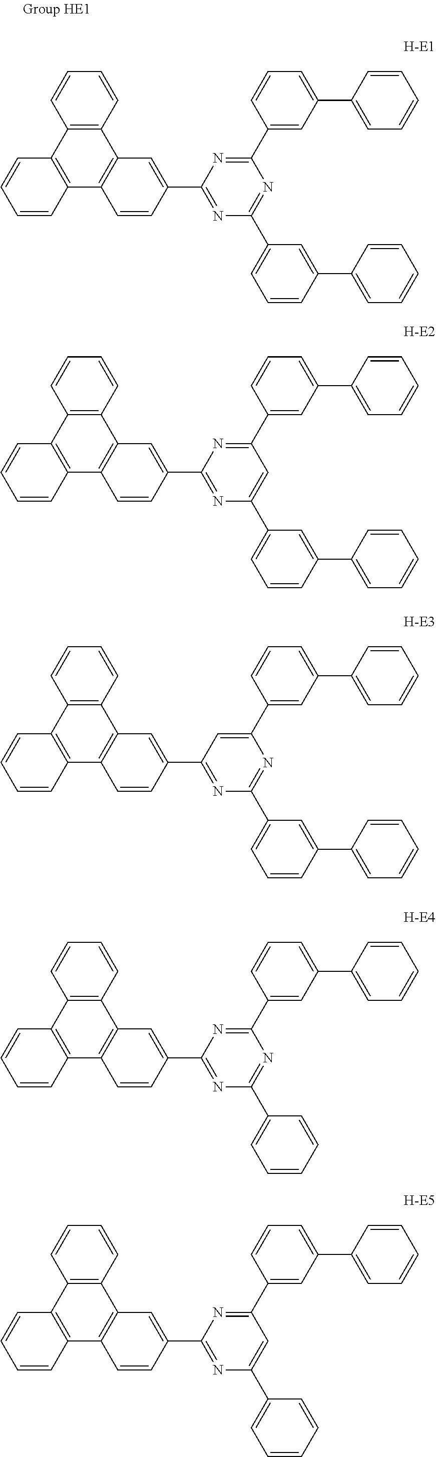 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2cccc(-c3cc(-c4cccc(-c5ccccc5)c4)nc(-c4ccc5c6ccccc6c6ccccc6c5c4)n3)c2)cc1 ; c1ccc(-c2cccc(-c3cc(-c4ccc5c6ccccc6c6ccccc6c5c4)nc(-c4cccc(-c5ccccc5)c4)n3)c2)cc1 ; c1ccc(-c2cccc(-c3nc(-c4ccccc4)nc(-c4ccc5c6ccccc6c6ccccc6c5c4)n3)c2)cc1 ; c1ccc(-c2cccc(-c3cc(-c4ccccc4)nc(-c4ccc5c6ccccc6c6ccccc6c5c4)n3)c2)cc1]

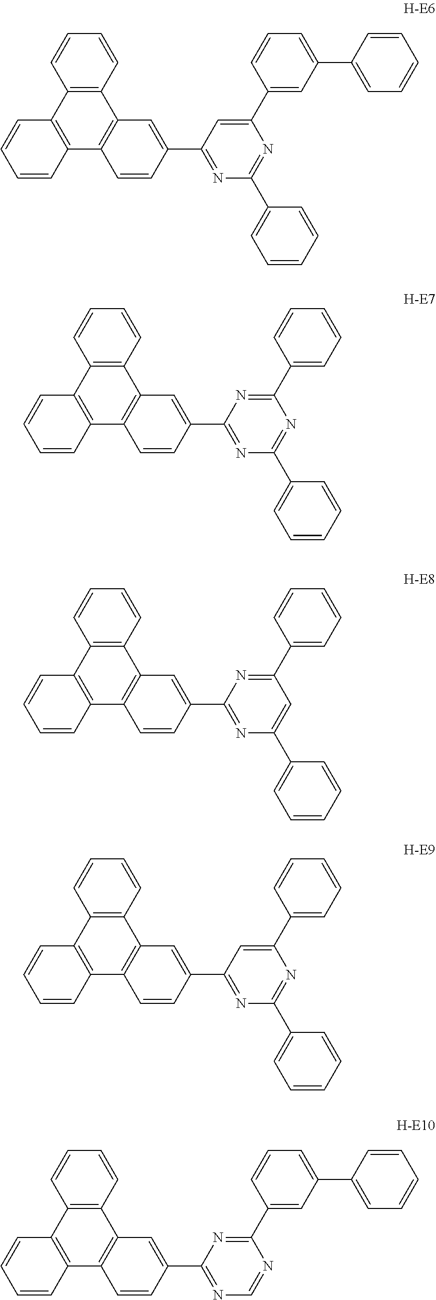

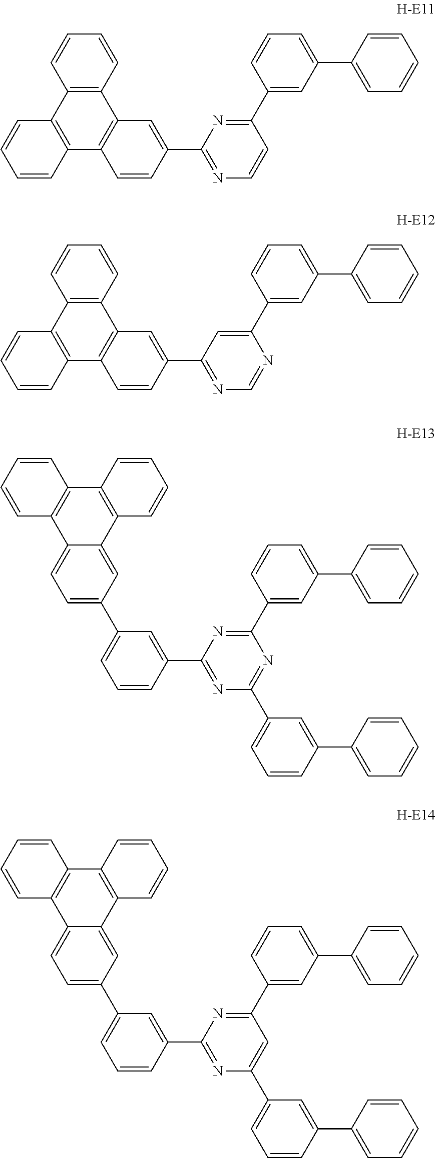 [SMILES: c1ccc(-c2cccc(-c3ccnc(-c4ccc5c6ccccc6c6ccccc6c5c4)n3)c2)cc1 ; c1ccc(-c2cccc(-c3cc(-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccc6c7ccccc7c7ccccc7c6c5)c4)n3)c2)cc1]

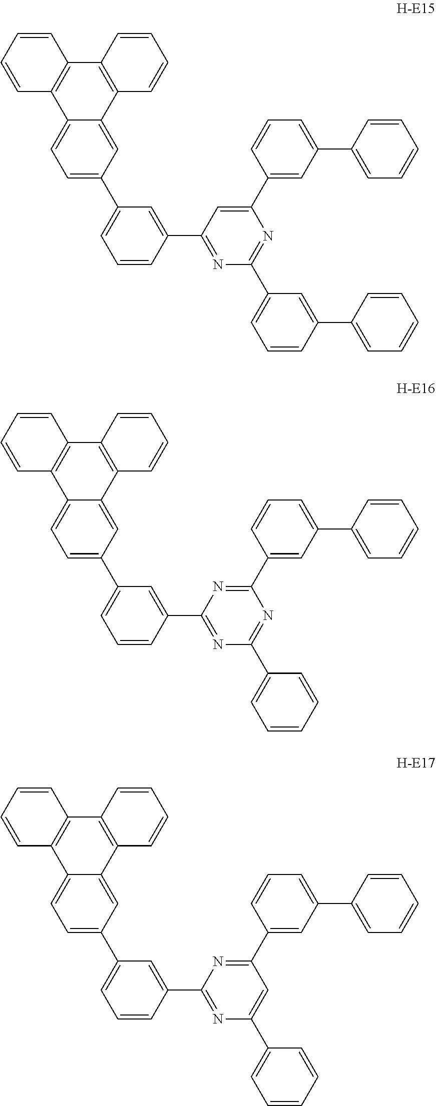 [SMILES: c1ccc(-c2cccc(-c3cc(-c4cccc(-c5ccc6c7ccccc7c7ccccc7c6c5)c4)nc(-c4cccc(-c5ccccc5)c4)n3)c2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cccc(-c3cc(-c4ccccc4)nc(-c4cccc(-c5ccc6c7ccccc7c7ccccc7c6c5)c4)n3)c2)cc1]

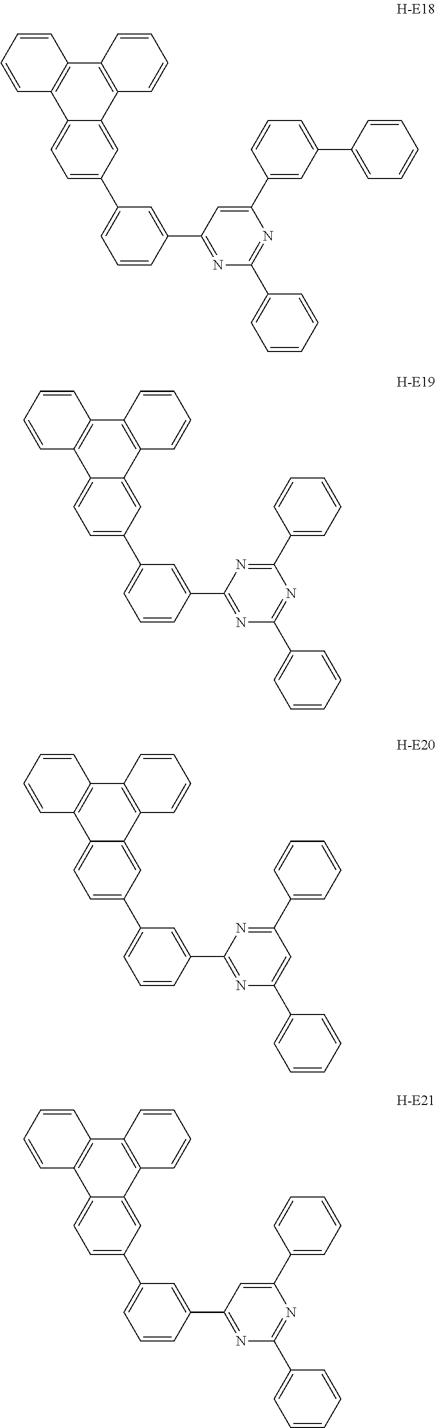 [SMILES: c1ccc(-c2cccc(-c3cc(-c4cccc(-c5ccc6c7ccccc7c7ccccc7c6c5)c4)nc(-c4ccccc4)n3)c2)cc1 ; c1ccc(-c2nc(-c3ccccc3)nc(-c3cccc(-c4ccc5c6ccccc6c6ccccc6c5c4)c3)n2)cc1 ; c1ccc(-c2cc(-c3ccccc3)nc(-c3cccc(-c4ccc5c6ccccc6c6ccccc6c5c4)c3)n2)cc1 ; c1ccc(-c2cc(-c3cccc(-c4ccc5c6ccccc6c6ccccc6c5c4)c3)nc(-c3ccccc3)n2)cc1]

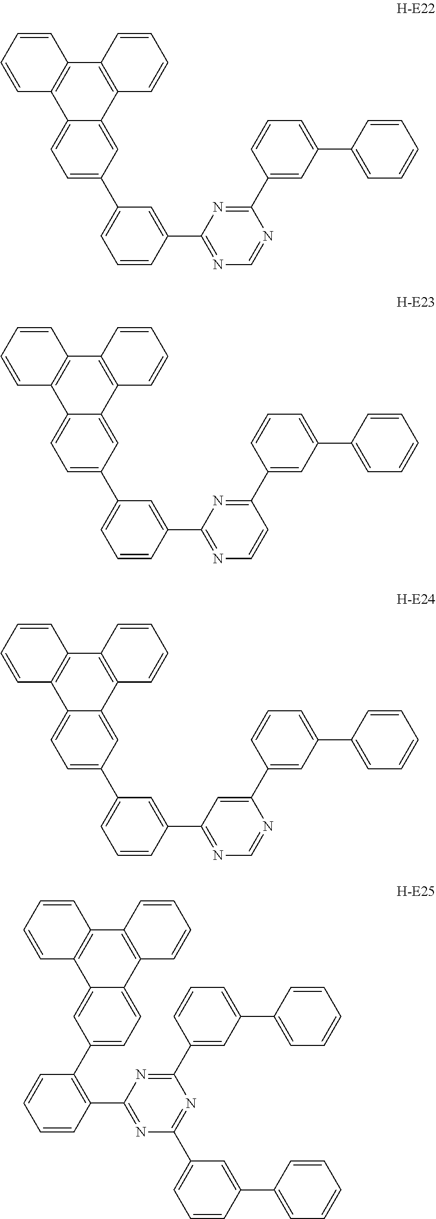 [SMILES: c1ccc(-c2cccc(-c3ncnc(-c4cccc(-c5ccc6c7ccccc7c7ccccc7c6c5)c4)n3)c2)cc1 ; c1ccc(-c2cccc(-c3ccnc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)ncn3)c2)cc1 ; c1ccc(-c2cccc(-c3nc(-c4cccc(-c5ccccc5)c4)nc(-c4ccccc4-c4ccc5c6ccccc6c6ccccc6c5c4)n3)c2)cc1]

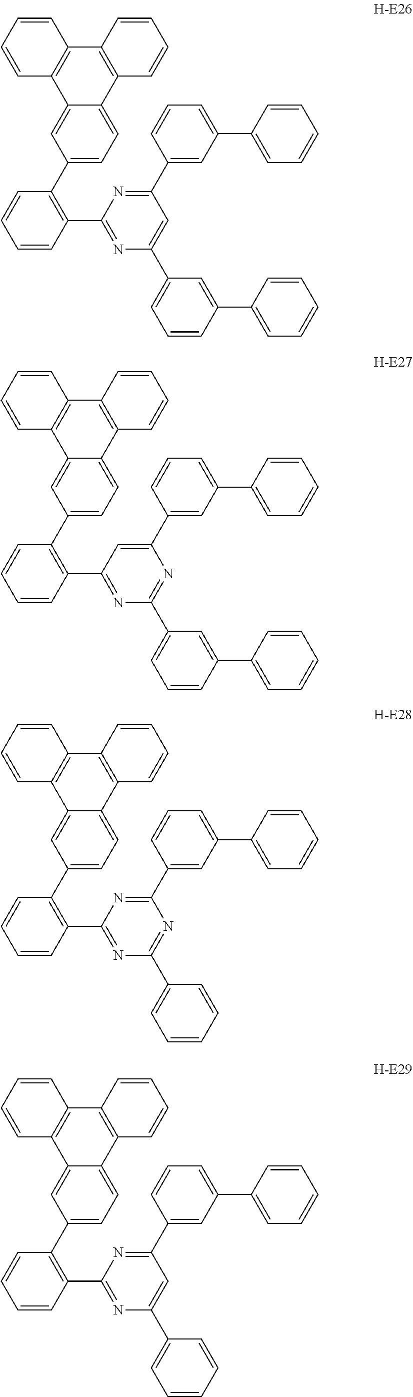

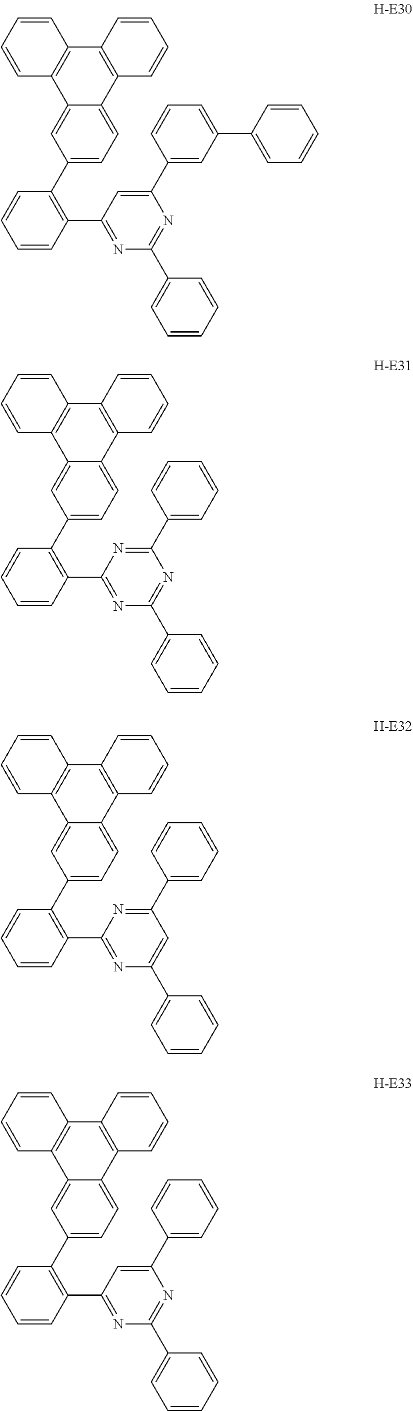

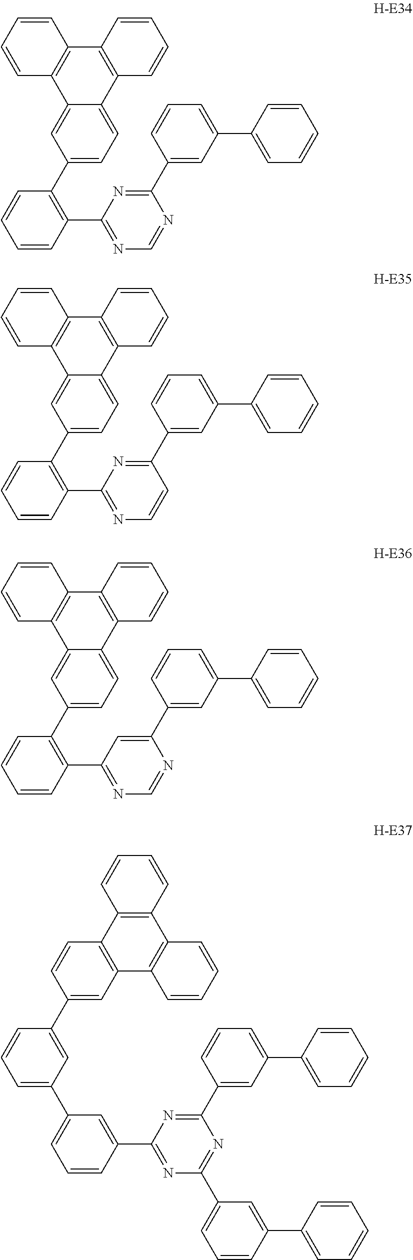

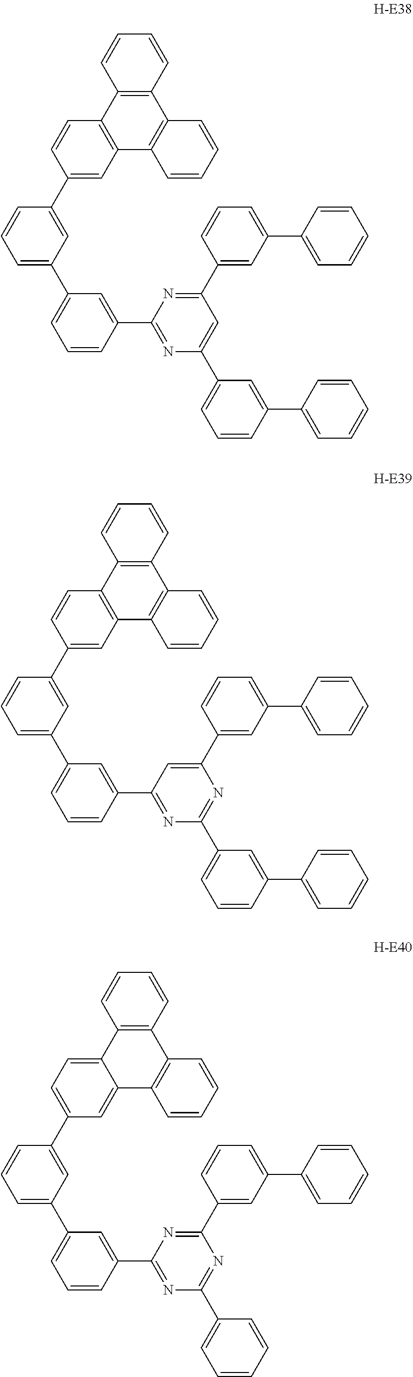 [SMILES: c1ccc(-c2cccc(-c3cc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4cccc(-c5ccccc5)c4)n3)c2)cc1 ; c1ccc(-c2cccc(-c3nc(-c4ccccc4)nc(-c4cccc(-c5cccc(-c6ccc7c8ccccc8c8ccccc8c7c6)c5)c4)n3)c2)cc1]

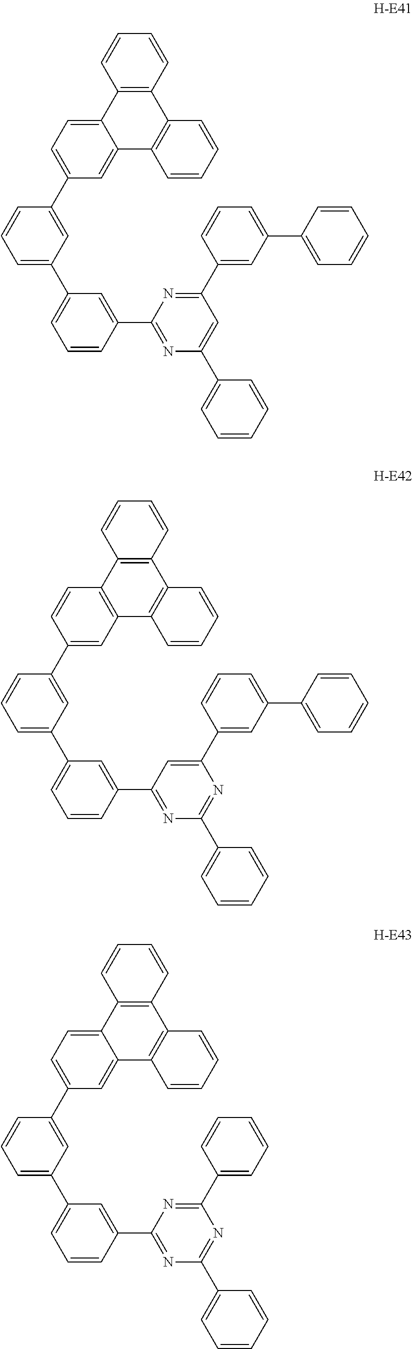 [SMILES: c1ccc(-c2cccc(-c3cc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4ccccc4)n3)c2)cc1 ; c1ccc(-c2nc(-c3ccccc3)nc(-c3cccc(-c4cccc(-c5ccc6c7ccccc7c7ccccc7c6c5)c4)c3)n2)cc1]

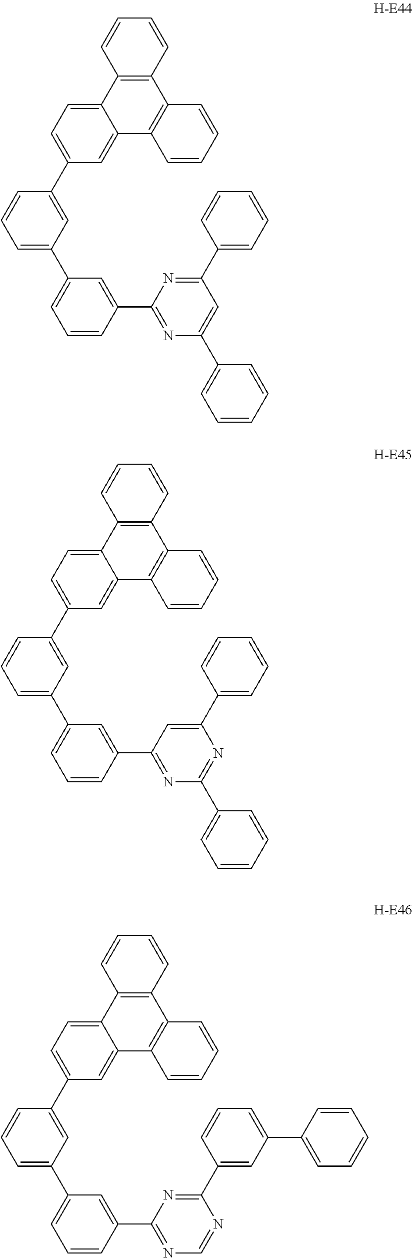 [SMILES: c1ccc(-c2cc(-c3ccccc3)nc(-c3cccc(-c4cccc(-c5ccc6c7ccccc7c7ccccc7c6c5)c4)c3)n2)cc1 ; c1ccc(-c2cc(-c3cccc(-c4cccc(-c5ccc6c7ccccc7c7ccccc7c6c5)c4)c3)nc(-c3ccccc3)n2)cc1 ; c1ccc(-c2cccc(-c3ncnc(-c4cccc(-c5cccc(-c6ccc7c8ccccc8c8ccccc8c7c6)c5)c4)n3)c2)cc1]

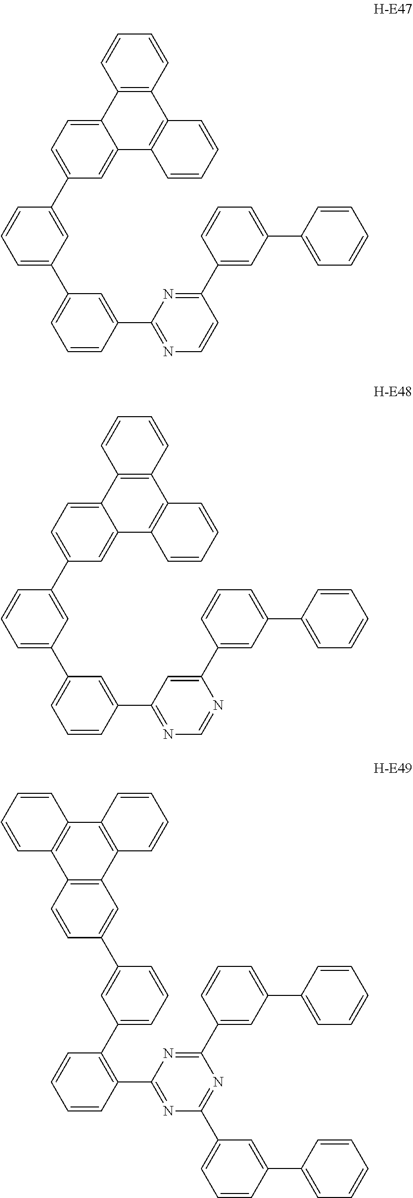 [SMILES: c1ccc(-c2cccc(-c3ccnc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)ncn3)c2)cc1 ; c1ccc(-c2cccc(-c3nc(-c4cccc(-c5ccccc5)c4)nc(-c4ccccc4-c4cccc(-c5ccc6c7ccccc7c7ccccc7c6c5)c4)n3)c2)cc1]

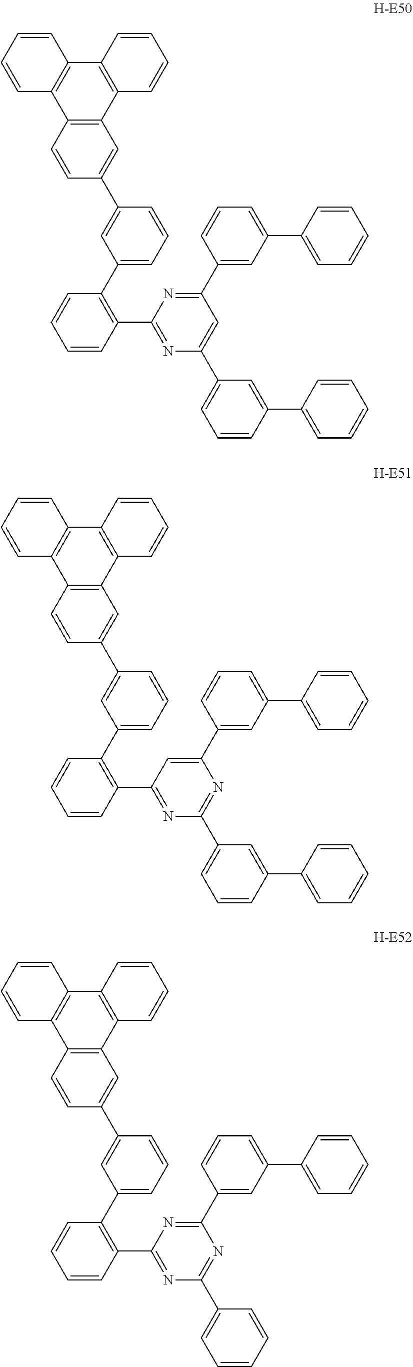 [SMILES: c1ccc(-c2cccc(-c3cc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4cccc(-c5ccccc5)c4)n3)c2)cc1 ; c1ccc(-c2cccc(-c3nc(-c4ccccc4)nc(-c4ccccc4-c4cccc(-c5ccc6c7ccccc7c7ccccc7c6c5)c4)n3)c2)cc1]

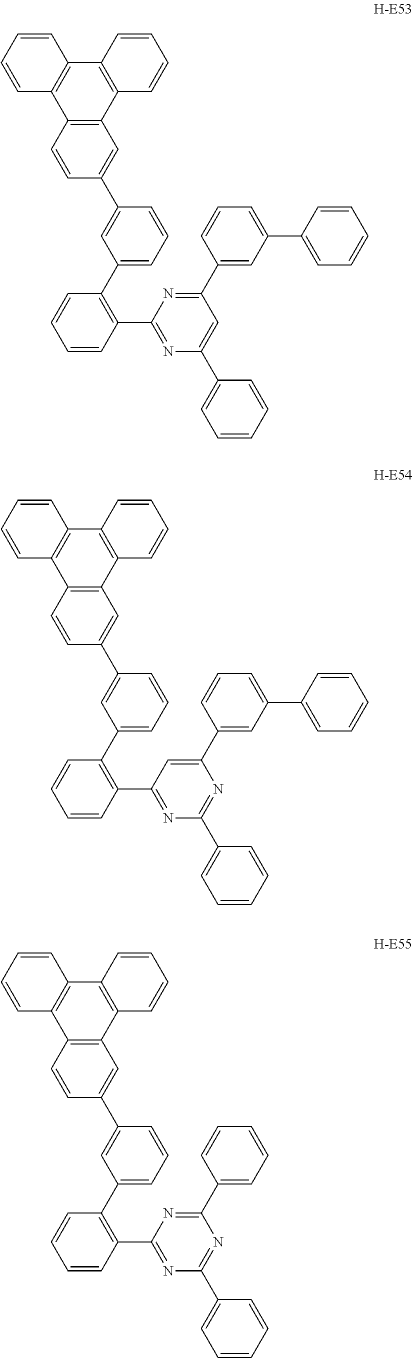 [SMILES: c1ccc(-c2cccc(-c3cc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4ccccc4)n3)c2)cc1 ; c1ccc(-c2nc(-c3ccccc3)nc(-c3ccccc3-c3cccc(-c4ccc5c6ccccc6c6ccccc6c5c4)c3)n2)cc1]

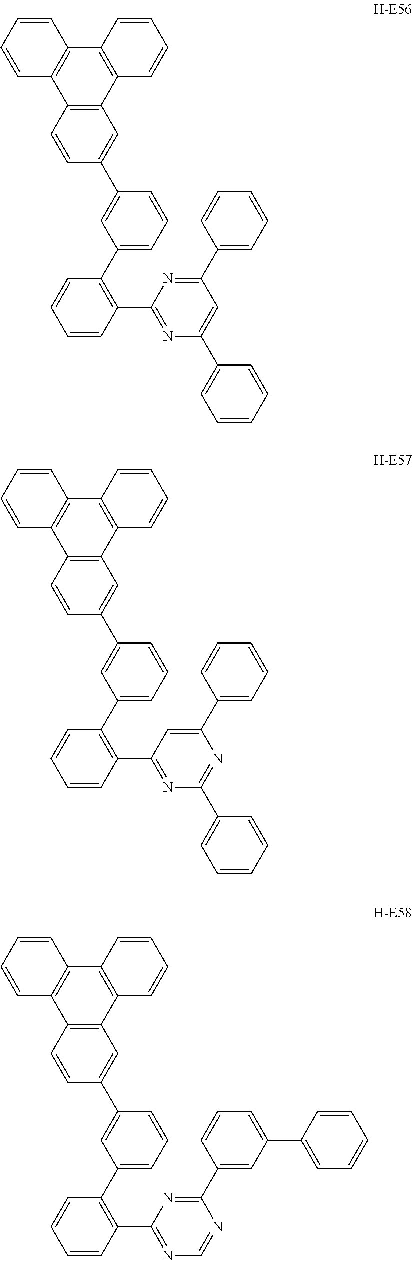 [SMILES: c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3ccccc3-c3cccc(-c4ccc5c6ccccc6c6ccccc6c5c4)c3)nc(-c3ccccc3)n2)cc1 ; c1ccc(-c2cccc(-c3ncnc(-c4ccccc4-c4cccc(-c5ccc6c7ccccc7c7ccccc7c6c5)c4)n3)c2)cc1]

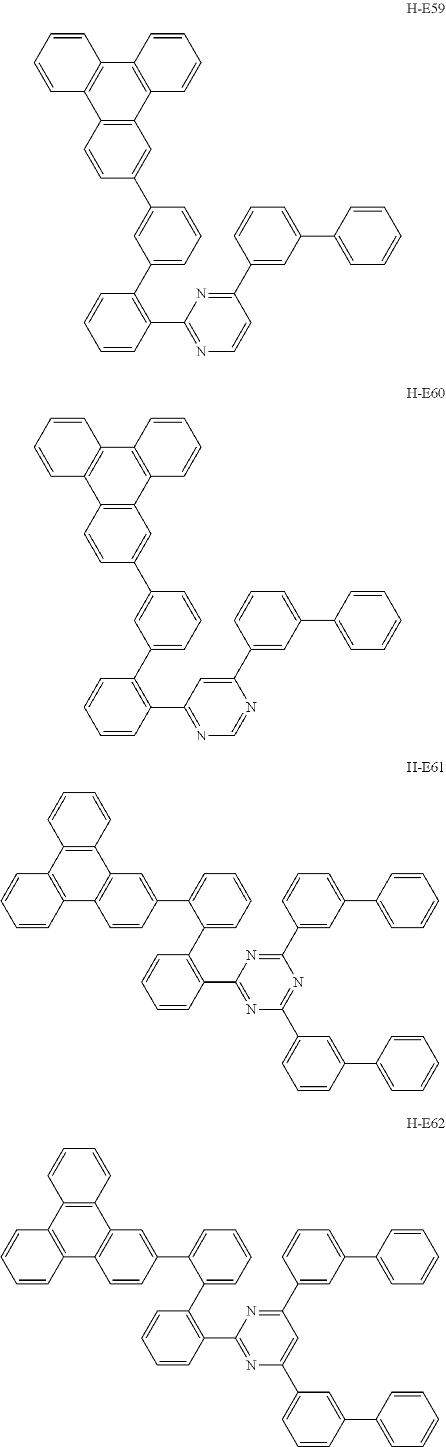 [SMILES: c1ccc(-c2cccc(-c3ccnc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)ncn3)c2)cc1 ; c1ccc(-c2cccc(-c3nc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4cccc(-c5ccccc5)c4)nc(-c4ccccc4-c4ccccc4-c4ccc5c6ccccc6c6ccccc6c5c4)n3)c2)cc1]

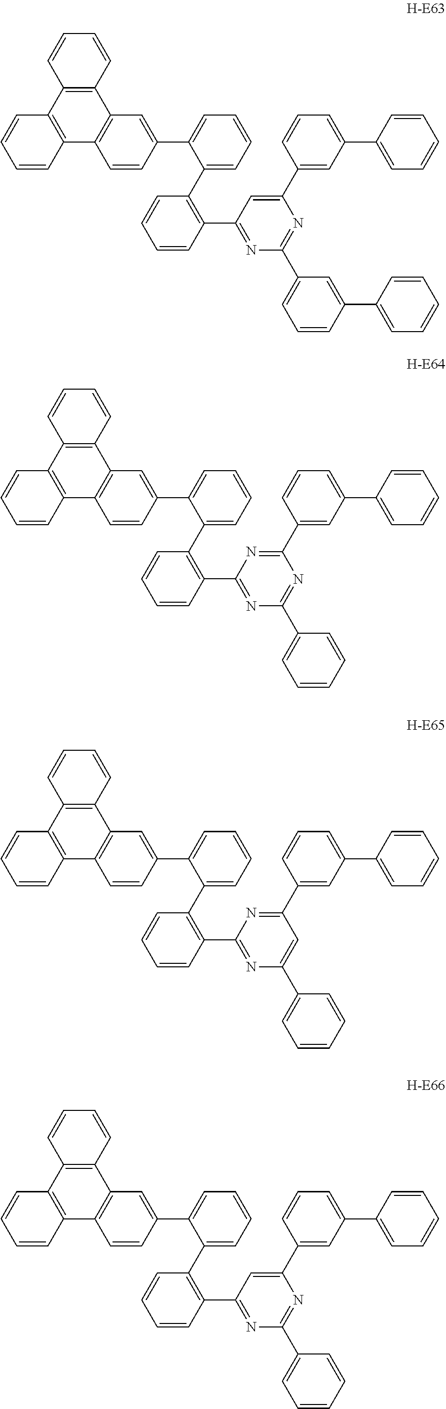 [SMILES: c1ccc(-c2cccc(-c3cc(-c4ccccc4-c4ccccc4-c4ccc5c6ccccc6c6ccccc6c5c4)nc(-c4cccc(-c5ccccc5)c4)n3)c2)cc1 ; c1ccc(-c2cccc(-c3nc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4ccccc4)n3)c2)cc1]

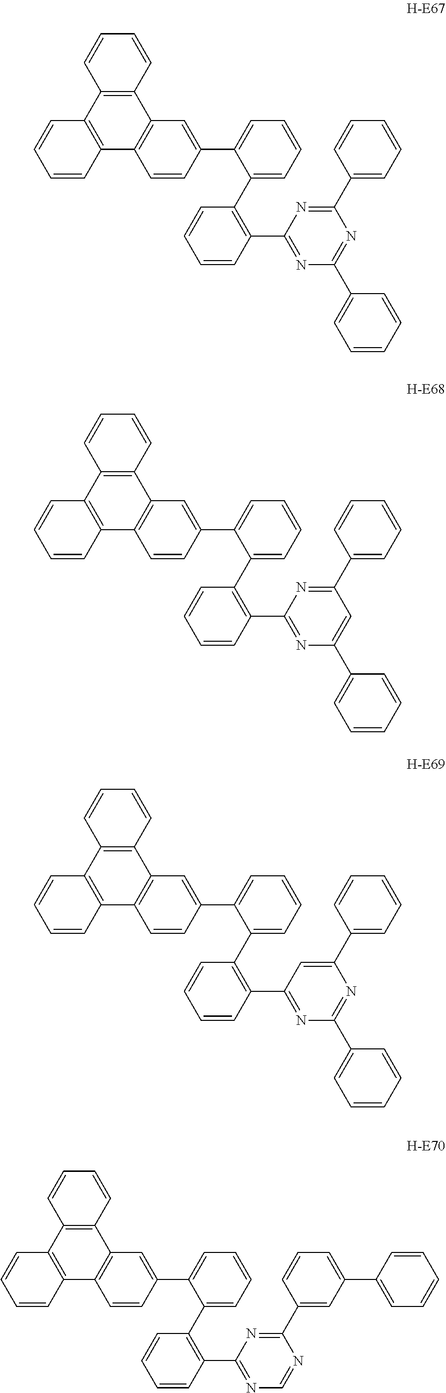 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2cc(-c3ccccc3-c3ccccc3-c3ccc4c5ccccc5c5ccccc5c4c3)nc(-c3ccccc3)n2)cc1 ; c1ccc(-c2cccc(-c3ncnc(-c4ccccc4-c4ccccc4-c4ccc5c6ccccc6c6ccccc6c5c4)n3)c2)cc1]

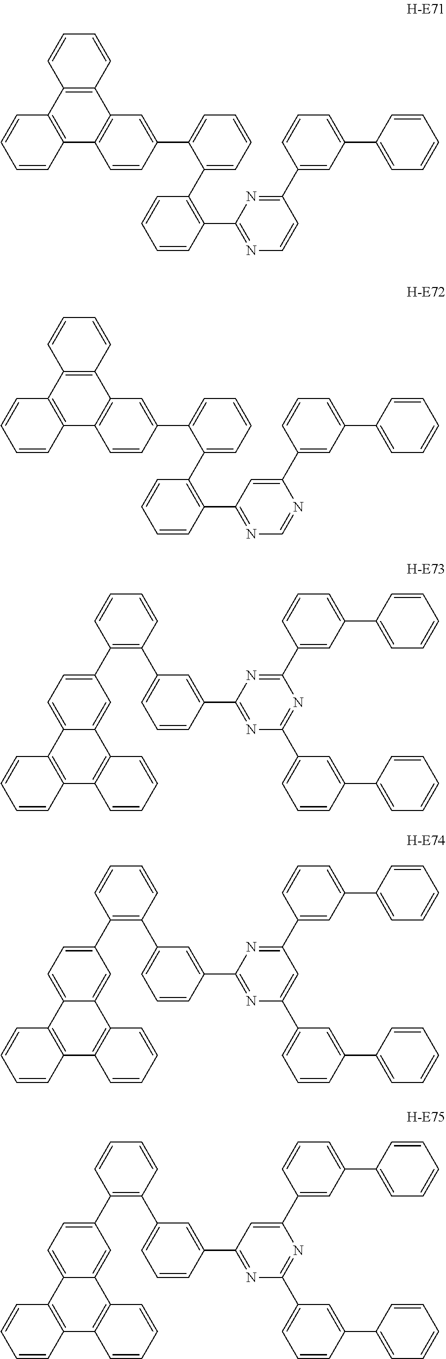 [SMILES: c1ccc(-c2cccc(-c3ccnc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4cccc(-c5ccccc5)c4)n3)c2)cc1]

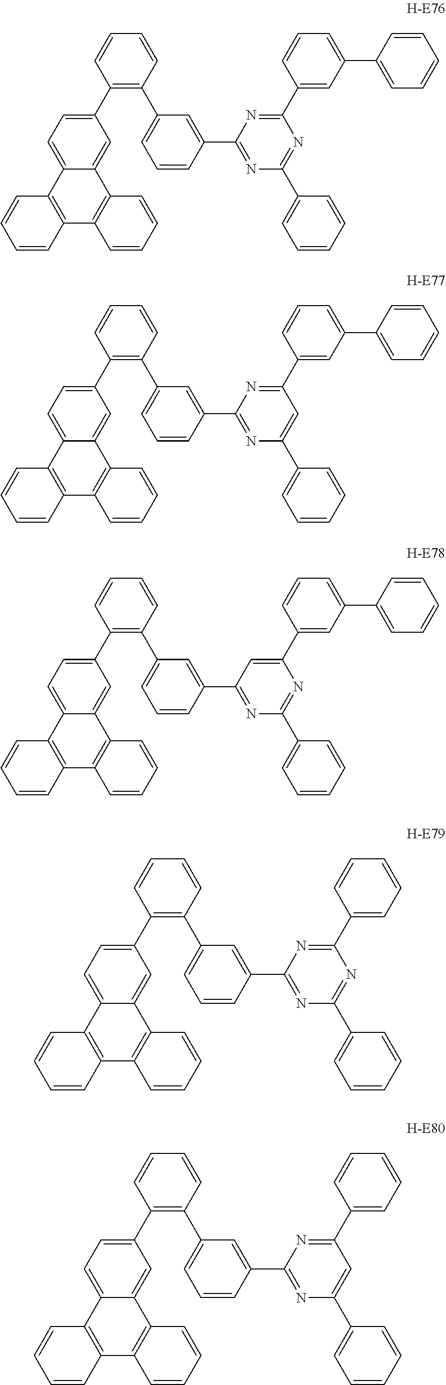 [SMILES: c1ccc(-c2cccc(-c3nc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4ccccc4)n3)c2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3cccc(-c4ccccc4-c4ccc5c6ccccc6c6ccccc6c5c4)c3)n2)cc1]

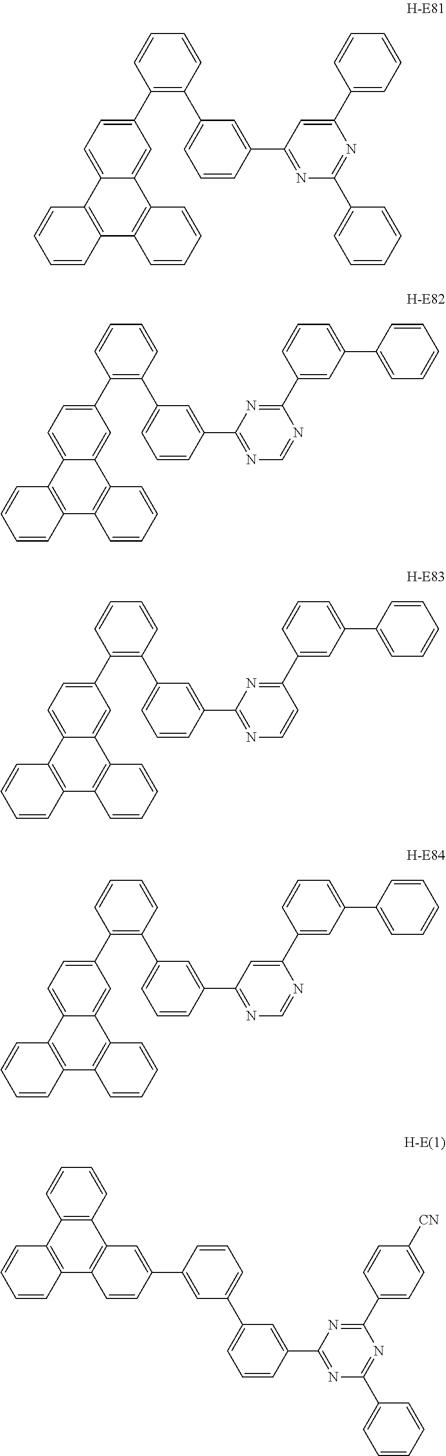 [SMILES: c1ccc(-c2cc(-c3cccc(-c4ccccc4-c4ccc5c6ccccc6c6ccccc6c5c4)c3)nc(-c3ccccc3)n2)cc1 ; c1ccc(-c2cccc(-c3ncnc(-c4cccc(-c5ccccc5-c5ccc6c7ccccc7c7ccccc7c6c5)c4)n3)c2)cc1 ; c1ccc(-c2cccc(-c3ccnc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)ncn3)c2)cc1 ; N#Cc1ccc(-c2nc(-c3ccccc3)nc(-c3cccc(-c4cccc(-c5ccc6c7ccccc7c7ccccc7c6c5)c4)c3)n2)cc1]

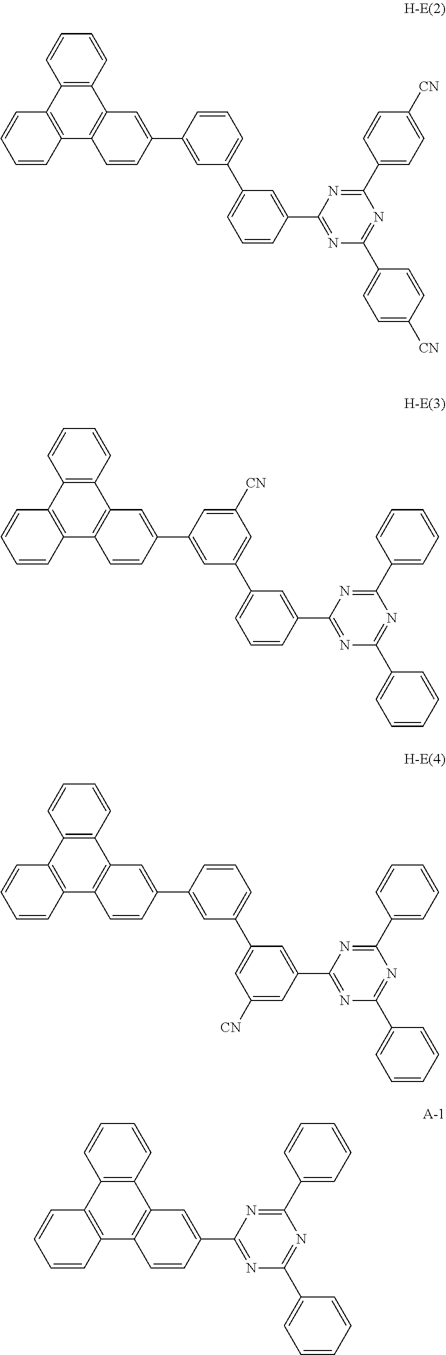

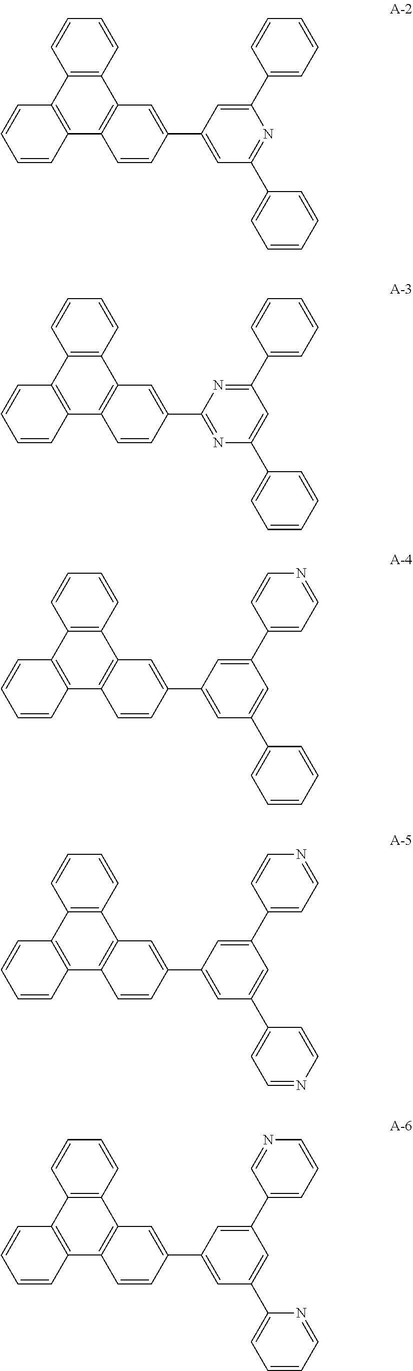

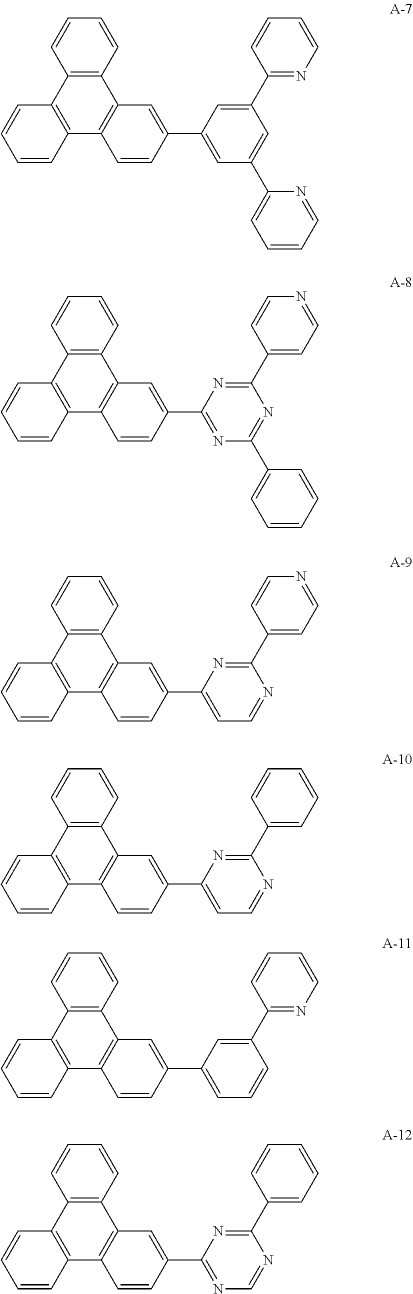

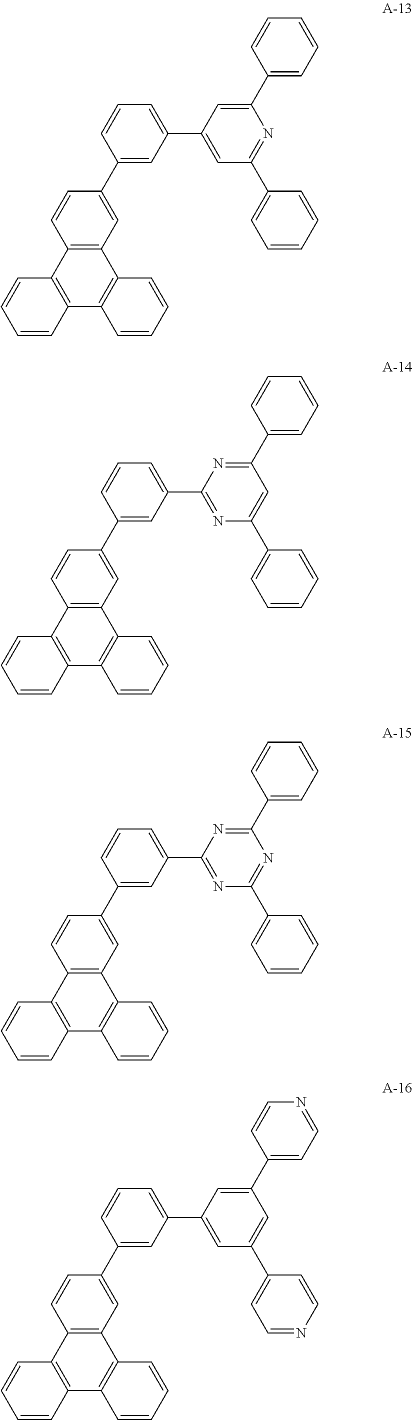 [SMILES: c1ccc(-c2cc(-c3cccc(-c4ccc5c6ccccc6c6ccccc6c5c4)c3)cc(-c3ccccc3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2cc(-c3ccncc3)cc(-c3ccncc3)c2)cc(-c2ccc3c4ccccc4c4ccccc4c3c2)c1]

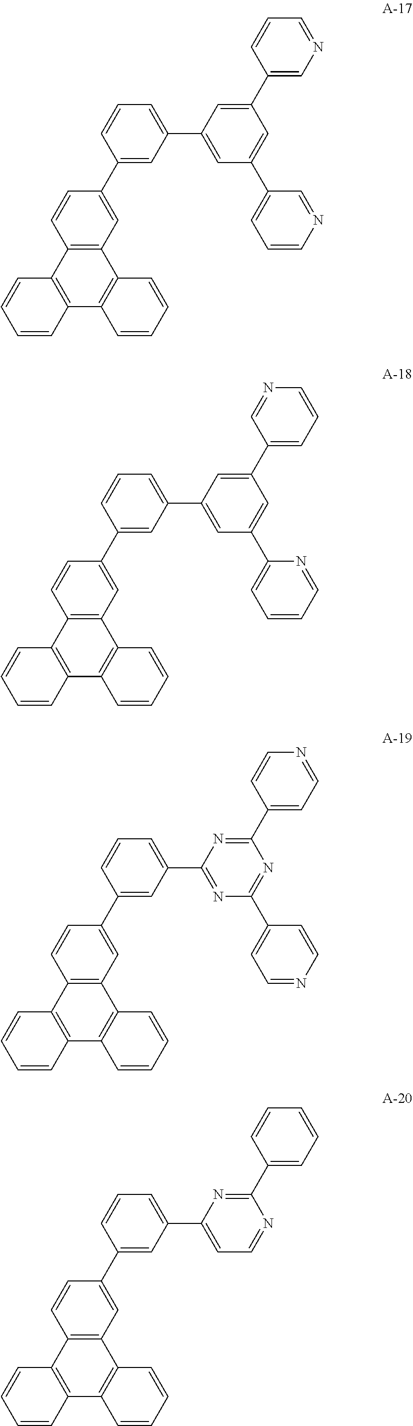 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

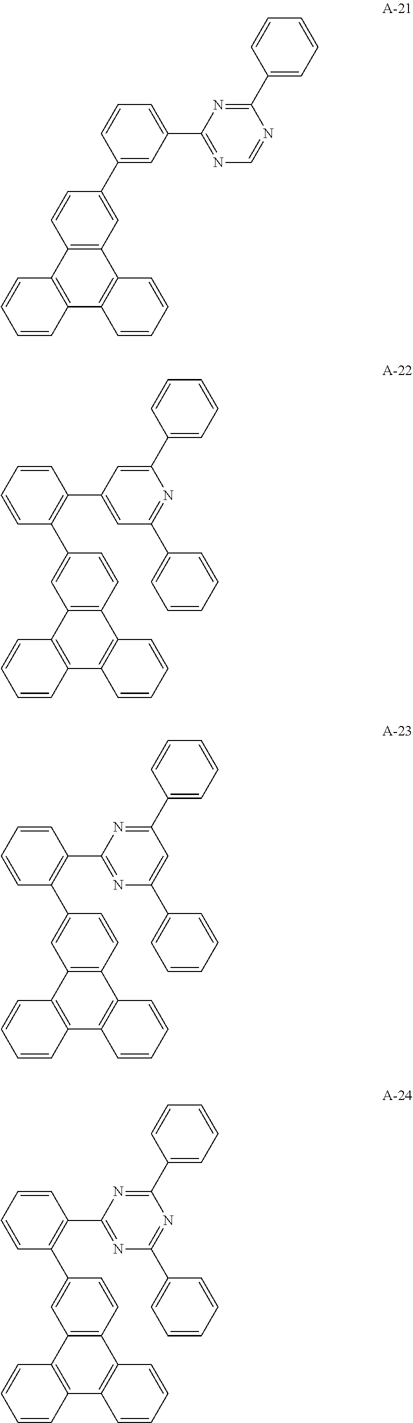

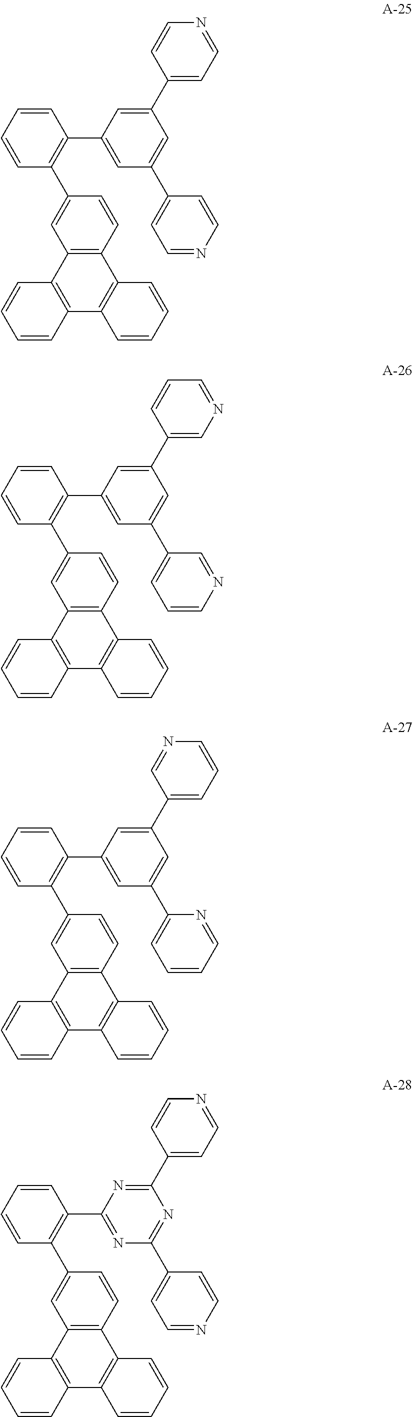

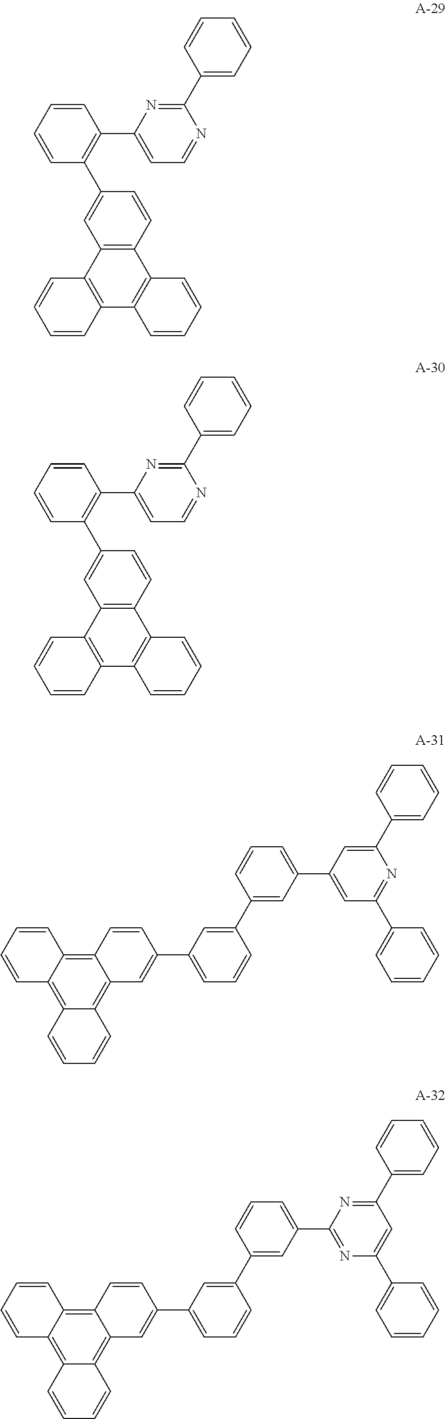 [SMILES: c1ccc(-c2nccc(-c3ccccc3-c3ccc4c5ccccc5c5ccccc5c4c3)n2)cc1 ; c1ccc(-c2nccc(-c3ccccc3-c3ccc4c5ccccc5c5ccccc5c4c3)n2)cc1 ; c1ccc(-c2cc(-c3cccc(-c4cccc(-c5ccc6c7ccccc7c7ccccc7c6c5)c4)c3)cc(-c3ccccc3)n2)cc1 ; c1ccc(-c2cc(-c3ccccc3)nc(-c3cccc(-c4cccc(-c5ccc6c7ccccc7c7ccccc7c6c5)c4)c3)n2)cc1]

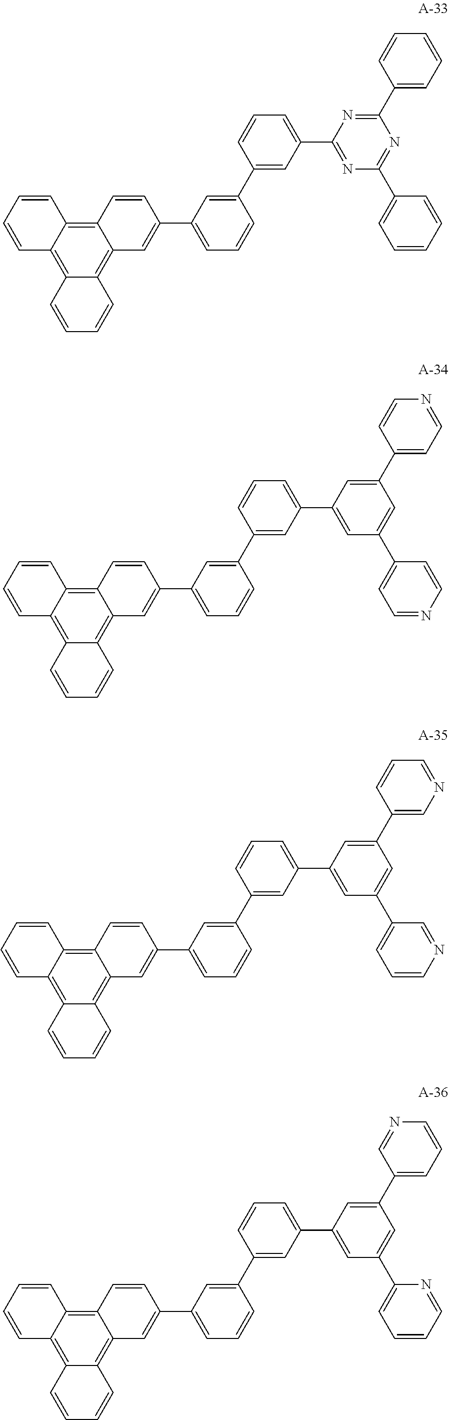 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1 ; c1ccc(-c2cc(-c3cccnc3)cc(-c3cccc(-c4cccc(-c5ccc6c7ccccc7c7ccccc7c6c5)c4)c3)c2)nc1]

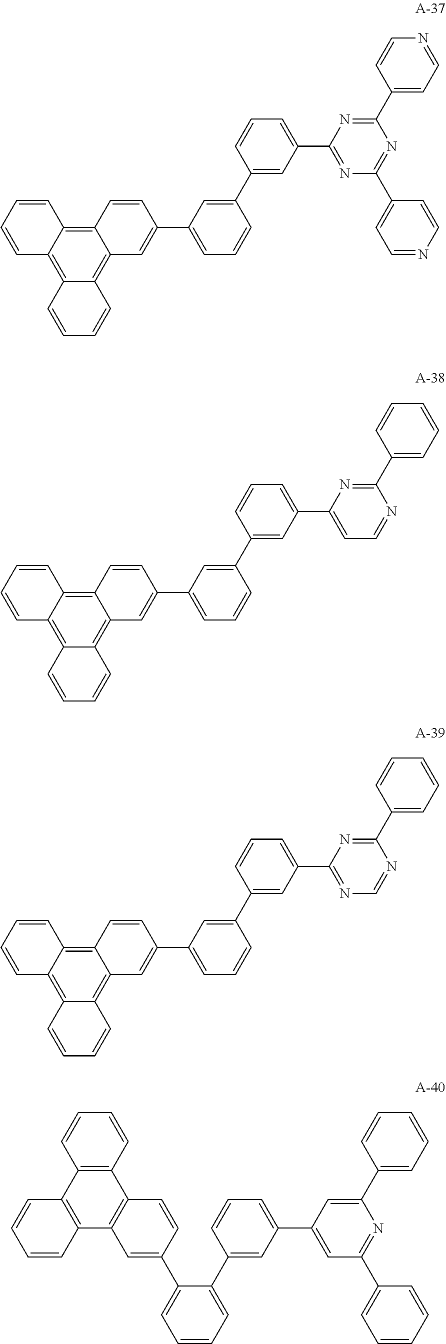 [SMILES: c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

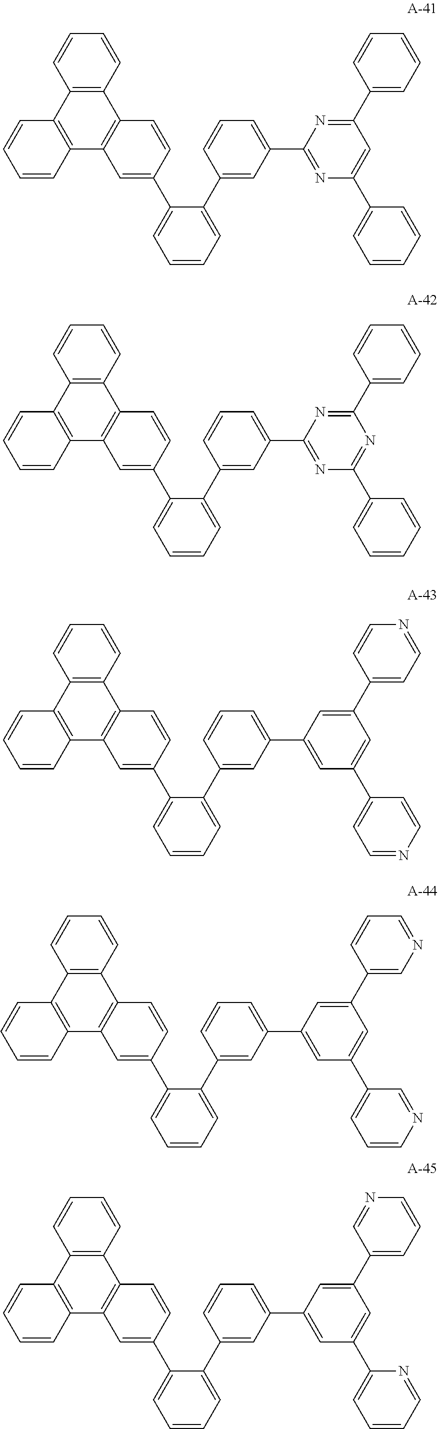 [SMILES: c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1cc(-c2cc(-c3ccncc3)cc(-c3ccncc3)c2)cc(-c2ccccc2-c2ccc3c4ccccc4c4ccccc4c3c2)c1 ; c1cncc(-c2cc(-c3cccnc3)cc(-c3cccc(-c4ccccc4-c4ccc5c6ccccc6c6ccccc6c5c4)c3)c2)c1 ; c1ccc(-c2cc(-c3cccnc3)cc(-c3cccc(-c4ccccc4-c4ccc5c6ccccc6c6ccccc6c5c4)c3)c2)nc1]

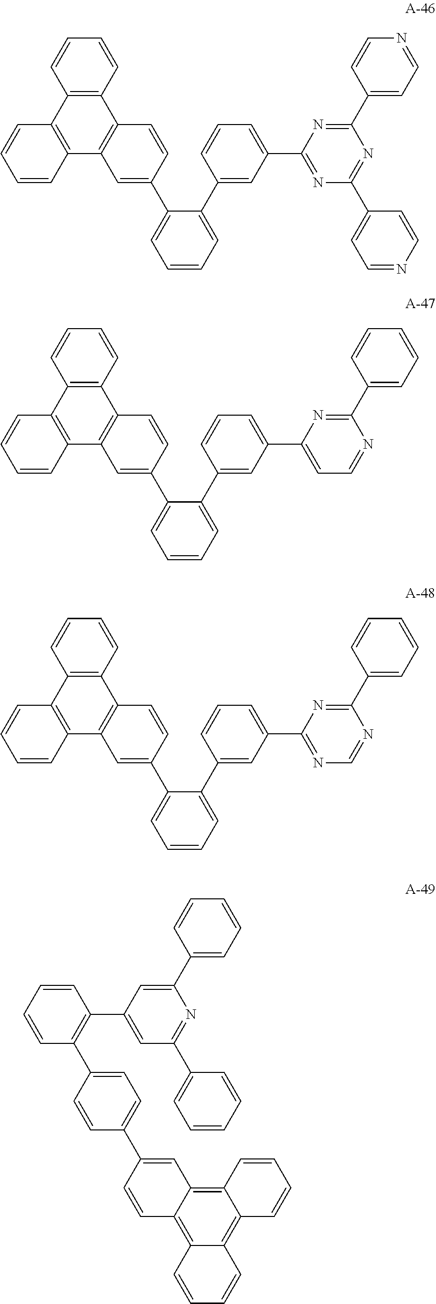 [SMILES: c1cc(-c2nc(-c3ccncc3)nc(-c3ccncc3)n2)cc(-c2ccccc2-c2ccc3c4ccccc4c4ccccc4c3c2)c1 ; c1ccc(-c2nccc(-c3cccc(-c4ccccc4-c4ccc5c6ccccc6c6ccccc6c5c4)c3)n2)cc1 ; c1ccc(-c2ncnc(-c3cccc(-c4ccccc4-c4ccc5c6ccccc6c6ccccc6c5c4)c3)n2)cc1 ; c1ccc(-c2cc(-c3ccccc3-c3ccc(-c4ccc5c6ccccc6c6ccccc6c5c4)cc3)cc(-c3ccccc3)n2)cc1]

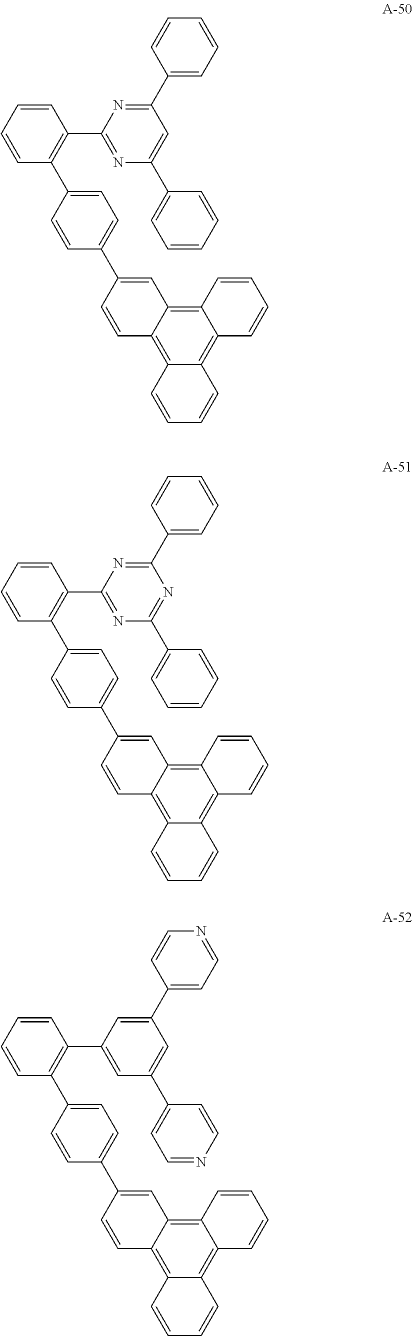 [SMILES: c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccncc3)cc(-c3ccncc3)c2)c(-c2ccc(-c3ccc4c5ccccc5c5ccccc5c4c3)cc2)c1]

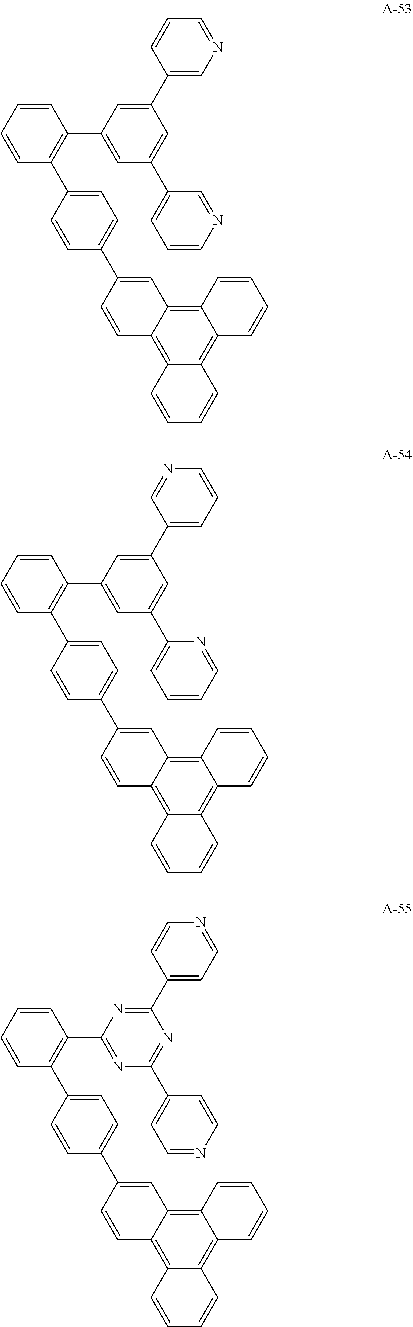

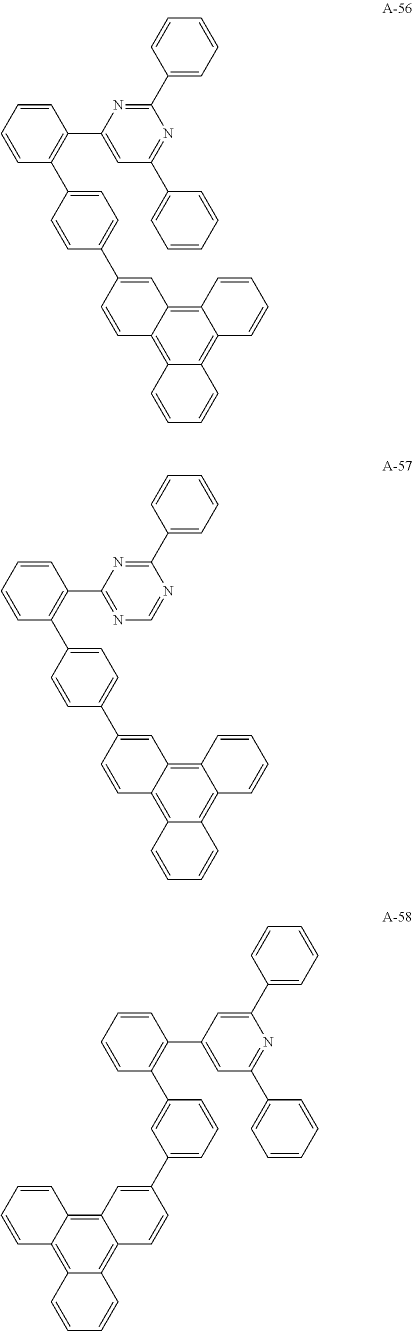 [SMILES: c1ccc(-c2cc(-c3ccccc3-c3ccc(-c4ccc5c6ccccc6c6ccccc6c5c4)cc3)nc(-c3ccccc3)n2)cc1 ; c1ccc(-c2ncnc(-c3ccccc3-c3ccc(-c4ccc5c6ccccc6c6ccccc6c5c4)cc3)n2)cc1 ; c1ccc(-c2cc(-c3ccccc3-c3cccc(-c4ccc5c6ccccc6c6ccccc6c5c4)c3)cc(-c3ccccc3)n2)cc1]

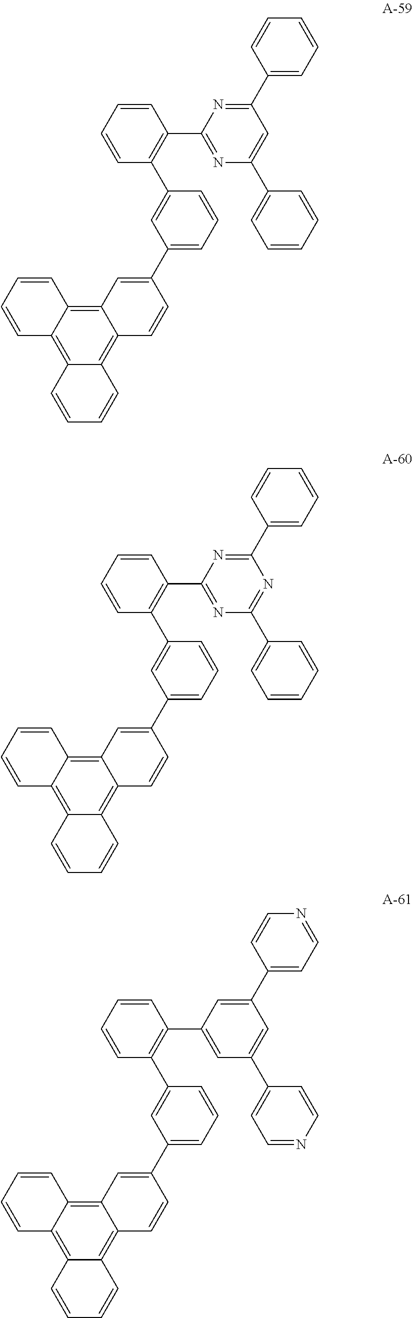 [SMILES: c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1]

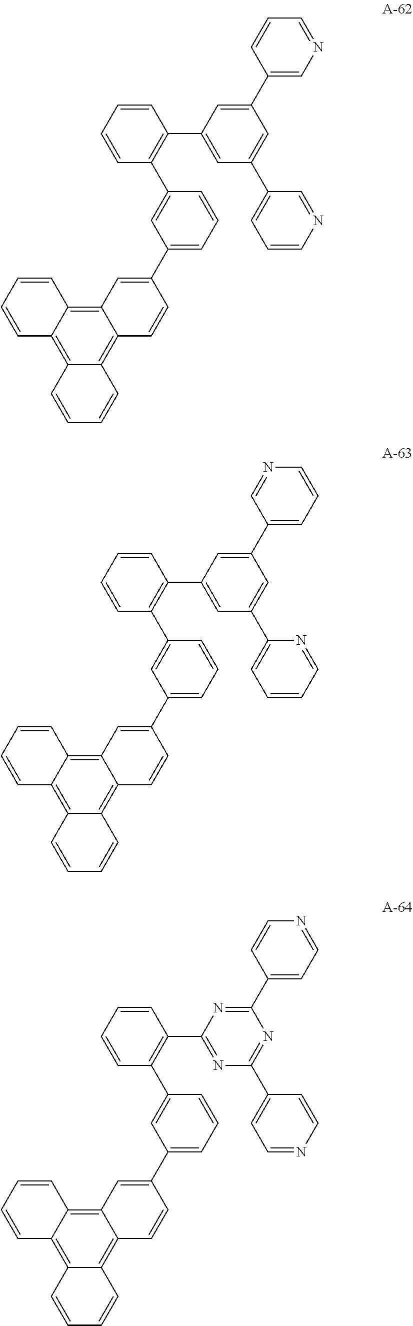 [SMILES: c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1]

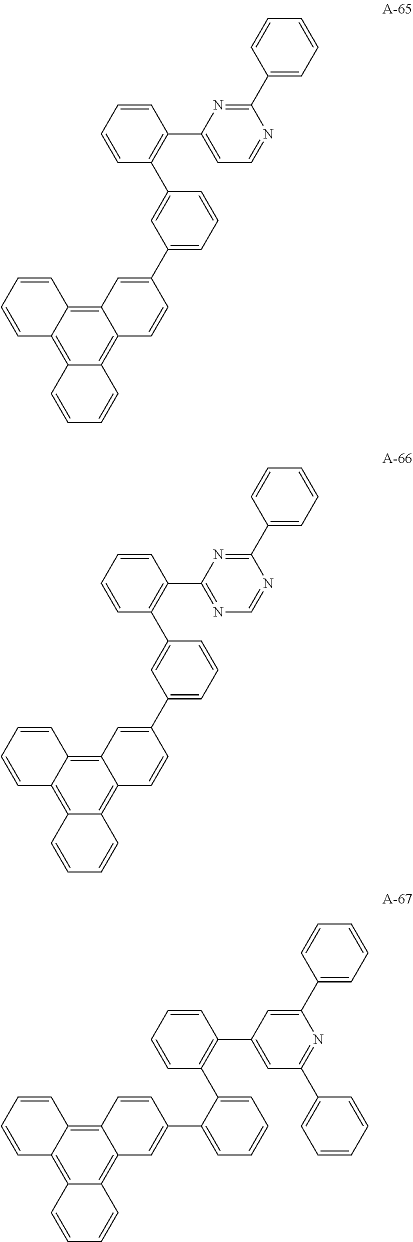 [SMILES: c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1]

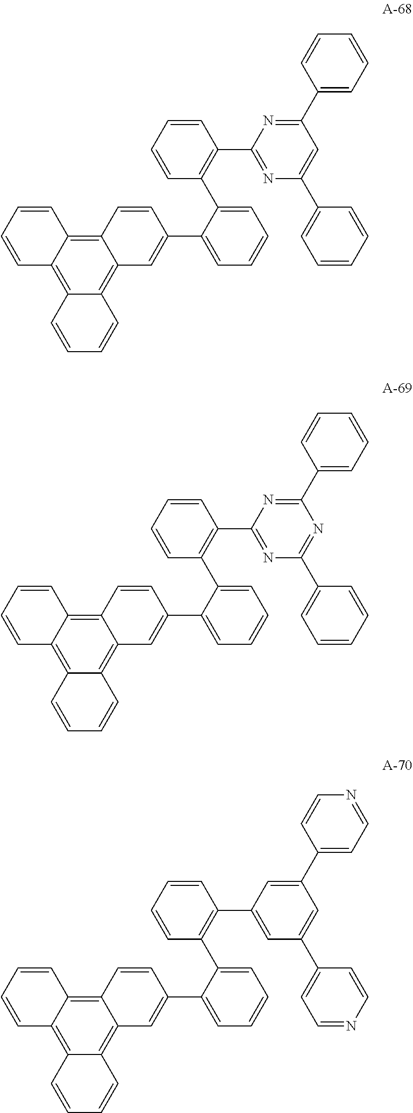 [SMILES: c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1]

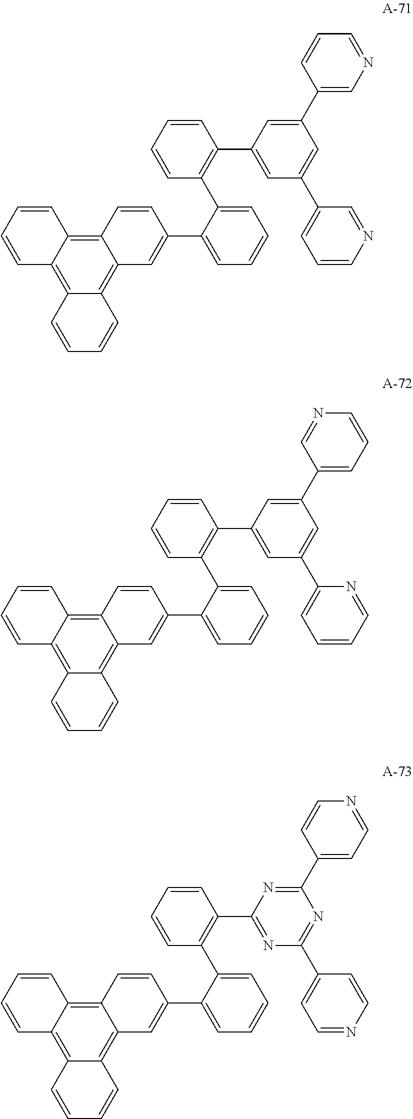 [SMILES: c1cncc(-c2cc(-c3cccnc3)cc(-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccc4c5ccccc5c5ccccc5c4c3)c2)nc1 ; c1ccc(-c2ccccc2-c2nc(-c3ccncc3)nc(-c3ccncc3)n2)c(-c2ccc3c4ccccc4c4ccccc4c3c2)c1]

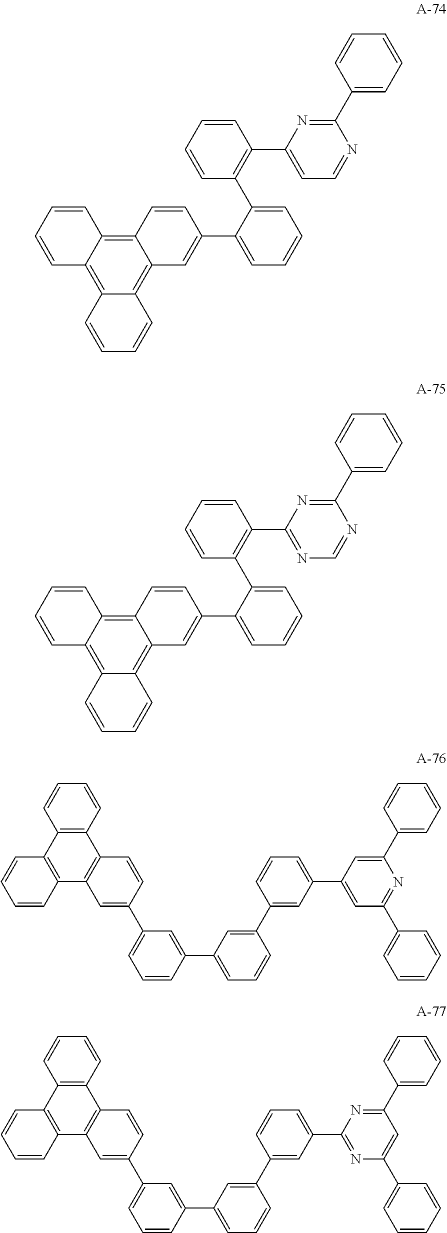 [SMILES: c1ccc(-c2nccc(-c3ccccc3-c3ccccc3-c3ccc4c5ccccc5c5ccccc5c4c3)n2)cc1 ; c1ccc(-c2ncnc(-c3ccccc3-c3ccccc3-c3ccc4c5ccccc5c5ccccc5c4c3)n2)cc1 ; c1ccc(-c2cc(-c3cccc(-c4cccc(-c5cccc(-c6ccc7c8ccccc8c8ccccc8c7c6)c5)c4)c3)cc(-c3ccccc3)n2)cc1 ; c1ccc(-c2cc(-c3ccccc3)nc(-c3cccc(-c4cccc(-c5cccc(-c6ccc7c8ccccc8c8ccccc8c7c6)c5)c4)c3)n2)cc1]

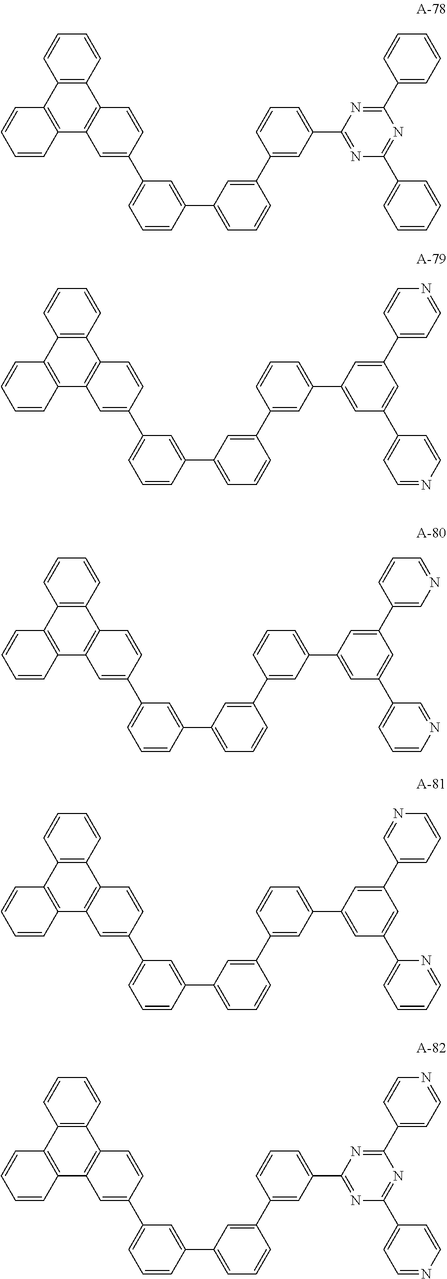 [SMILES: c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1cc(-c2cccc(-c3cc(-c4ccncc4)cc(-c4ccncc4)c3)c2)cc(-c2cccc(-c3ccc4c5ccccc5c5ccccc5c4c3)c2)c1 ; c1cncc(-c2cc(-c3cccnc3)cc(-c3cccc(-c4cccc(-c5cccc(-c6ccc7c8ccccc8c8ccccc8c7c6)c5)c4)c3)c2)c1 ; c1ccc(-c2cc(-c3cccnc3)cc(-c3cccc(-c4cccc(-c5cccc(-c6ccc7c8ccccc8c8ccccc8c7c6)c5)c4)c3)c2)nc1 ; c1cc(-c2cccc(-c3ccc4c5ccccc5c5ccccc5c4c3)c2)cc(-c2cccc(-c3nc(-c4ccncc4)nc(-c4ccncc4)n3)c2)c1]

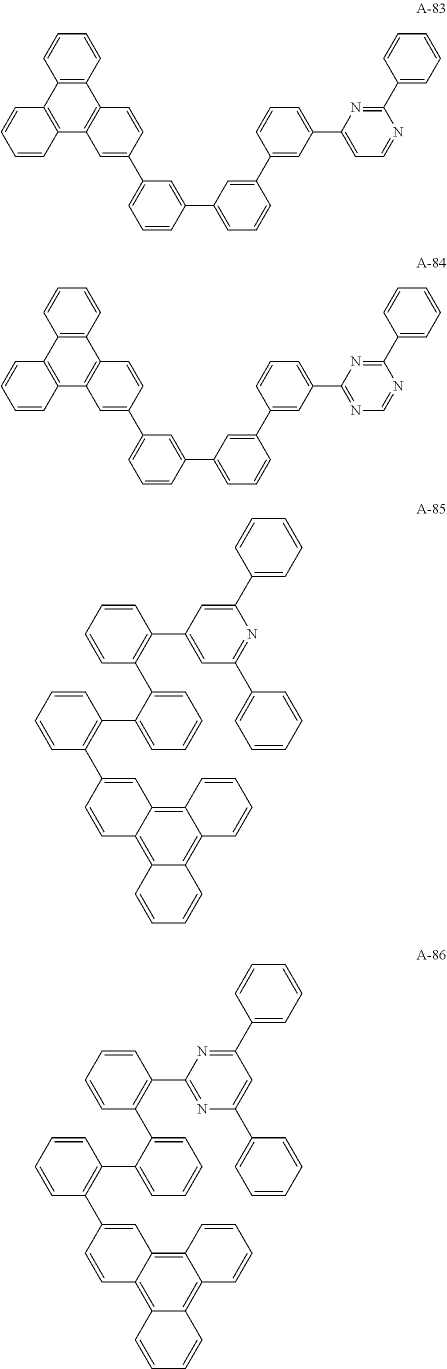

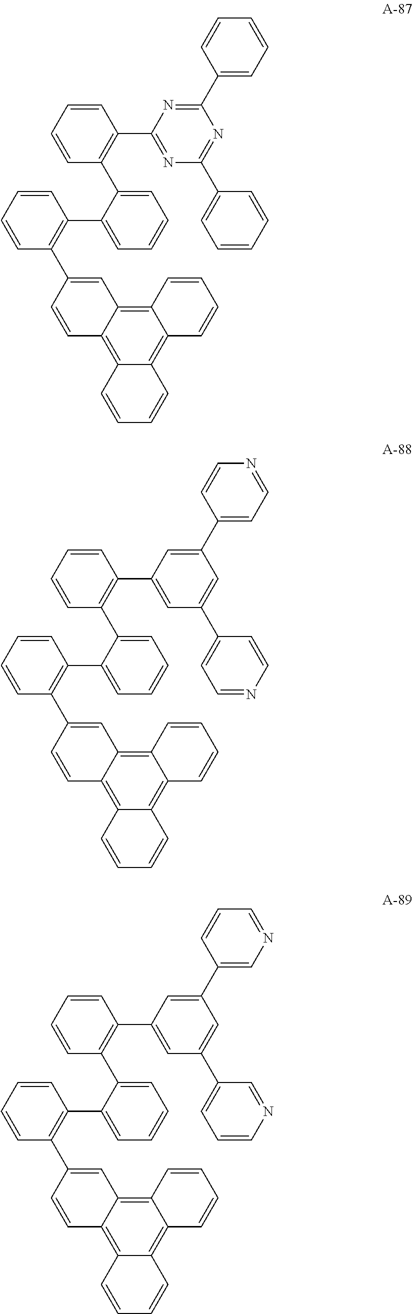 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccccc3-c3ccc4c5ccccc5c5ccccc5c4c3)c2)c1]

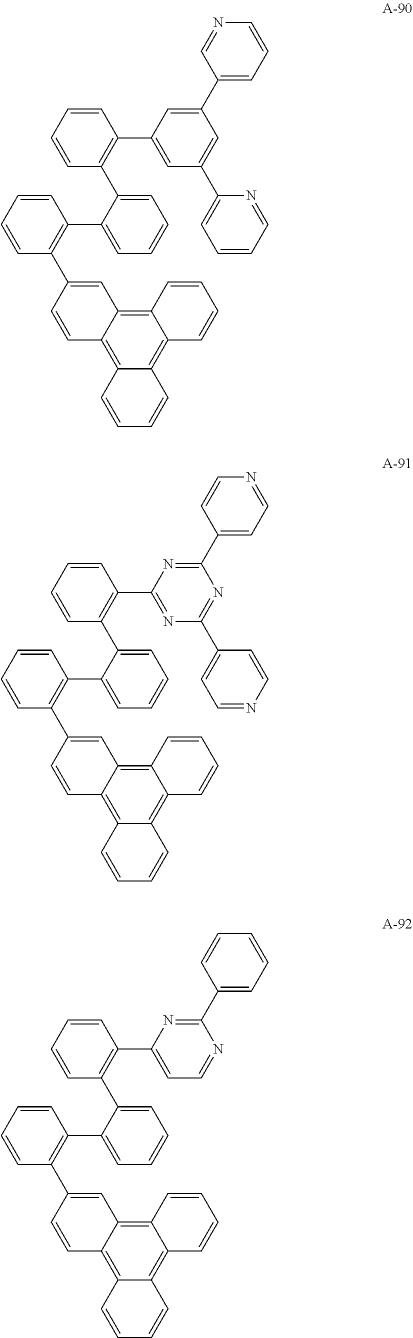 [SMILES: c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1]

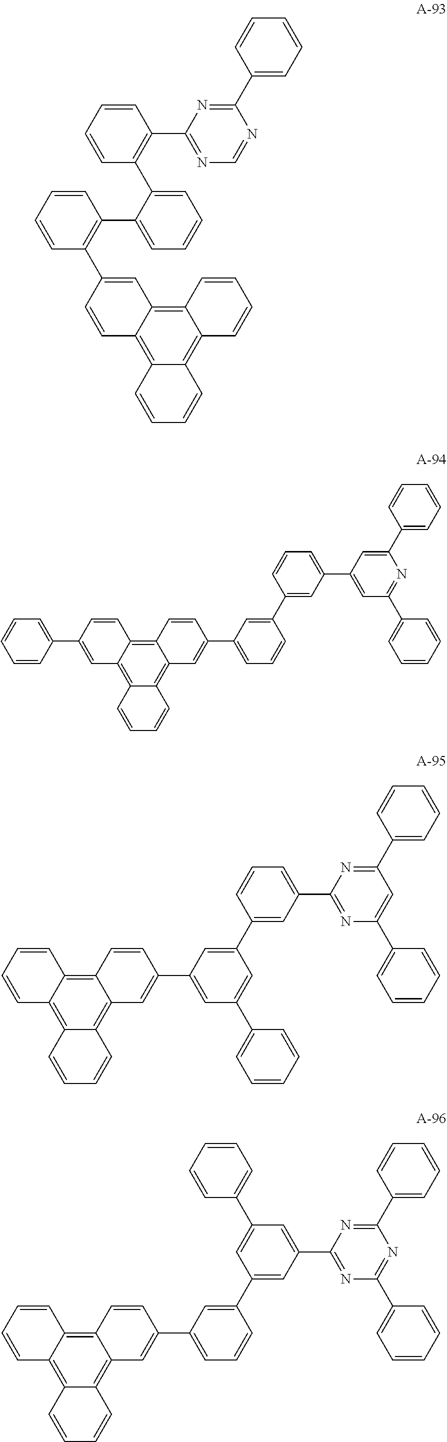 [SMILES: c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

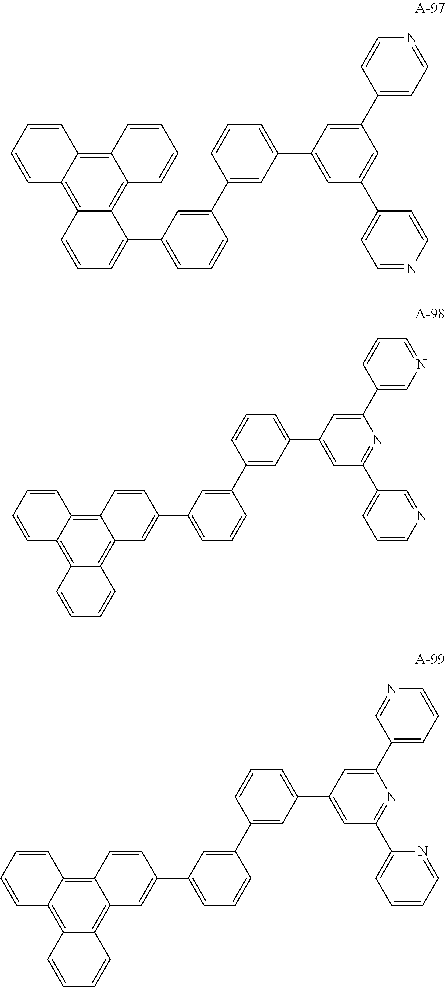 [SMILES: c1cc(-c2cc(-c3ccncc3)cc(-c3ccncc3)c2)cc(-c2cccc(-c3cccc4c5ccccc5c5ccccc5c34)c2)c1 ; c1cc(-c2cccc(-c3ccc4c5ccccc5c5ccccc5c4c3)c2)cc(-c2cc(-c3cccnc3)nc(-c3cccnc3)c2)c1 ; c1ccc(-c2cc(-c3cccc(-c4cccc(-c5ccc6c7ccccc7c7ccccc7c6c5)c4)c3)cc(-c3cccnc3)n2)nc1]

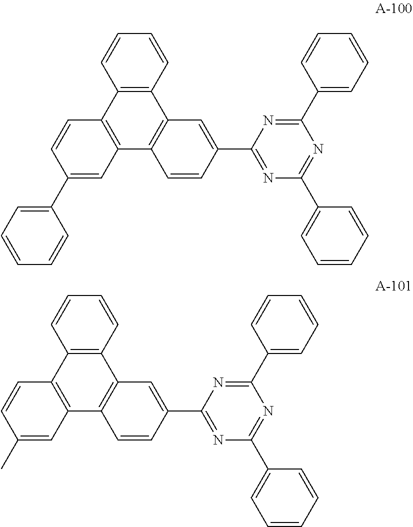

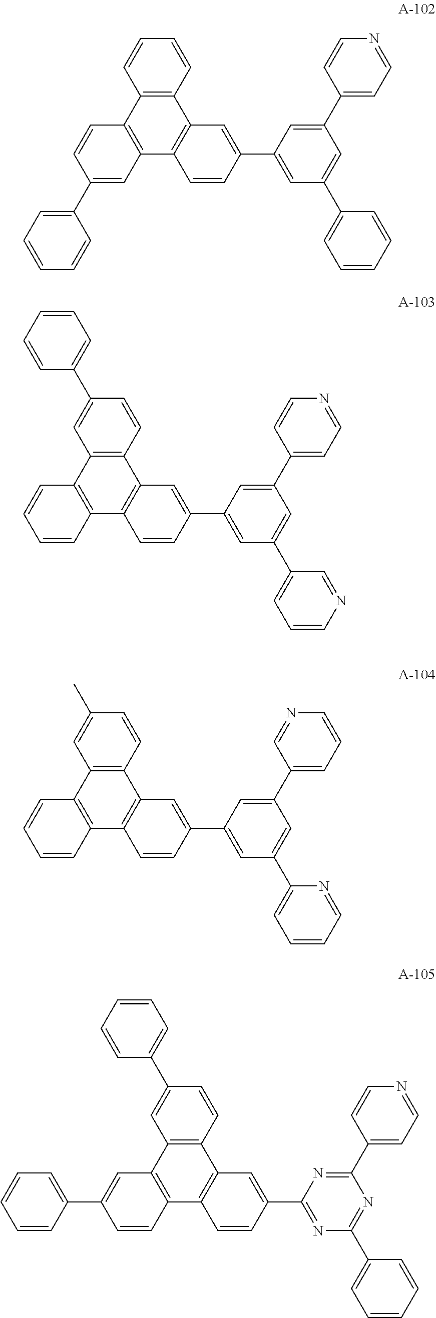

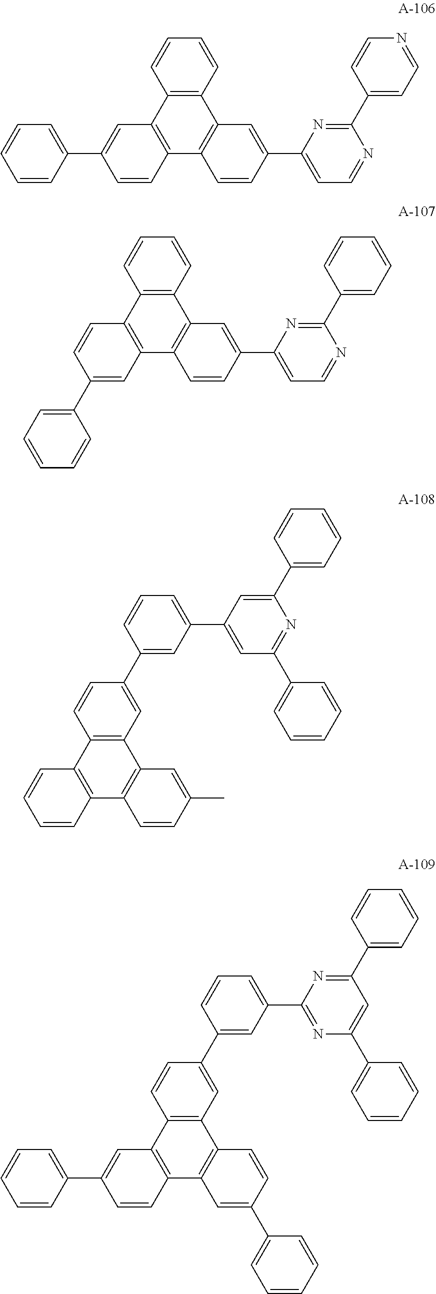 [SMILES: c1ccc(-c2ccc3c4ccc(-c5ccnc(-c6ccncc6)n5)cc4c4ccccc4c3c2)cc1 ; c1ccc(-c2ccc3c4ccccc4c4cc(-c5ccnc(-c6ccccc6)n5)ccc4c3c2)cc1 ; Cc1ccc2c3ccccc3c3ccc(-c4cccc(-c5cc(-c6ccccc6)nc(-c6ccccc6)c5)c4)cc3c2c1 ; c1ccc(-c2ccc3c(c2)c2ccc(-c4cccc(-c5nc(-c6ccccc6)cc(-c6ccccc6)n5)c4)cc2c2ccc(-c4ccccc4)cc32)cc1]

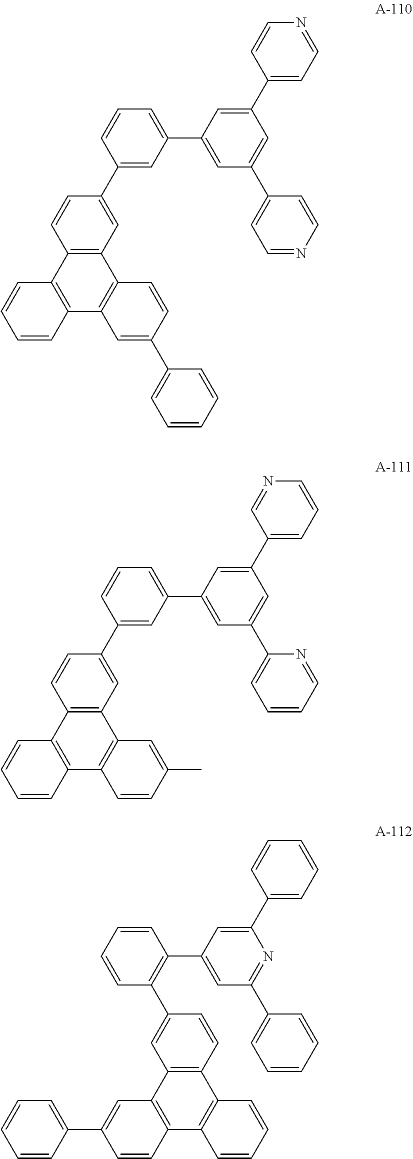 [SMILES: c1ccc(-c2ccc3c(c2)c2ccccc2c2ccc(-c4cccc(-c5cc(-c6ccncc6)cc(-c6ccncc6)c5)c4)cc23)cc1 ; Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1]

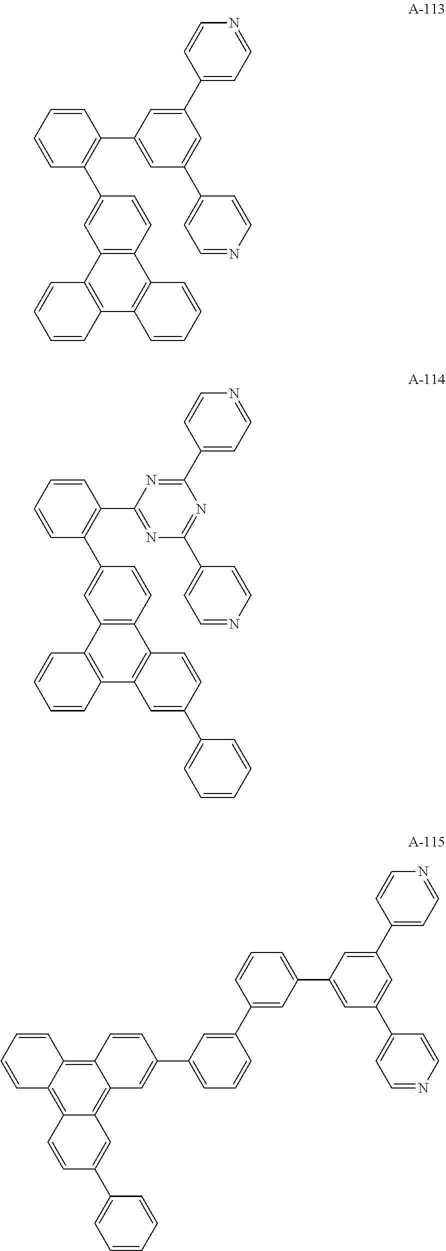 [SMILES: c1ccc(-c2ccc3c4ccccc4c4ccccc4c3c2)c(-c2cc(-c3ccncc3)cc(-c3ccncc3)c2)c1 ; c1ccc(-c2ccc3c4ccc(-c5ccccc5-c5nc(-c6ccncc6)nc(-c6ccncc6)n5)cc4c4ccccc4c3c2)cc1 ; c1ccc(-c2ccc3c4ccccc4c4ccc(-c5cccc(-c6cccc(-c7cc(-c8ccncc8)cc(-c8ccncc8)c7)c6)c5)cc4c3c2)cc1]

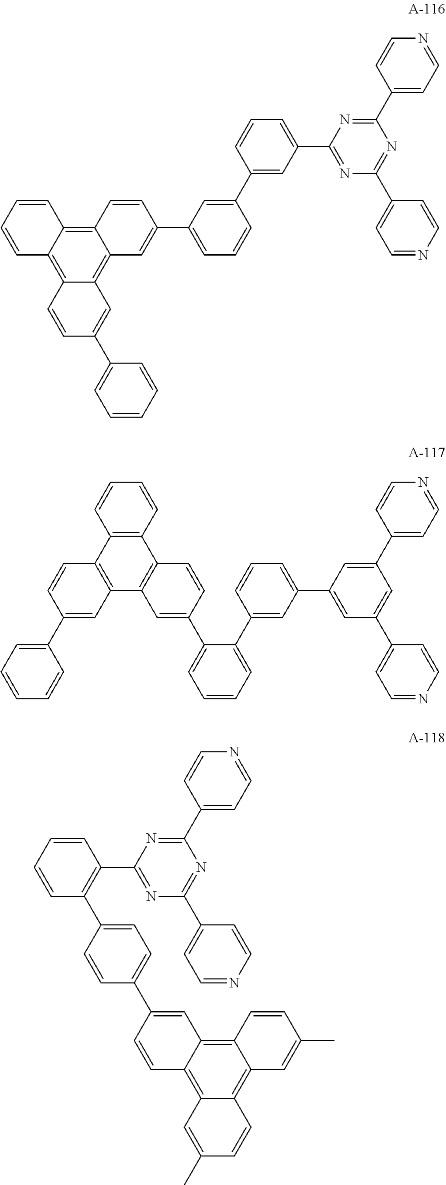 [SMILES: c1ccc(-c2ccc3c4ccccc4c4ccc(-c5cccc(-c6cccc(-c7nc(-c8ccncc8)nc(-c8ccncc8)n7)c6)c5)cc4c3c2)cc1 ; c1ccc(-c2ccc3c4ccccc4c4ccc(-c5ccccc5-c5cccc(-c6cc(-c7ccncc7)cc(-c7ccncc7)c6)c5)cc4c3c2)cc1 ; Cc1ccc2c(c1)c1ccc(C)cc1c1ccc(-c3ccc(-c4ccccc4-c4nc(-c5ccncc5)nc(-c5ccncc5)n4)cc3)cc21]

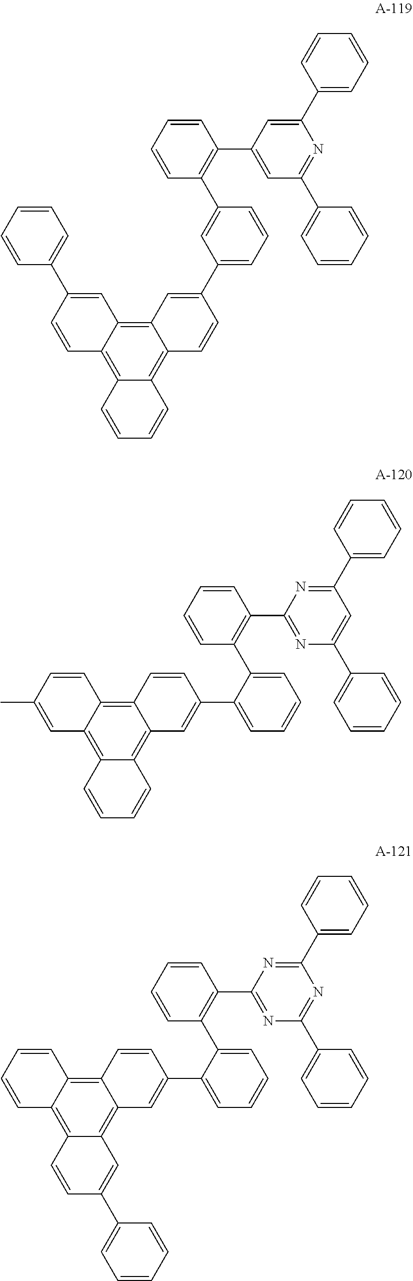 [SMILES: c1ccc(-c2ccc3c4ccccc4c4ccc(-c5cccc(-c6ccccc6-c6cc(-c7ccccc7)nc(-c7ccccc7)c6)c5)cc4c3c2)cc1 ; Cc1ccc2c3ccc(-c4ccccc4-c4ccccc4-c4nc(-c5ccccc5)cc(-c5ccccc5)n4)cc3c3ccccc3c2c1 ; c1ccc(-c2ccc3c4ccccc4c4ccc(-c5ccccc5-c5ccccc5-c5nc(-c6ccccc6)nc(-c6ccccc6)n5)cc4c3c2)cc1]

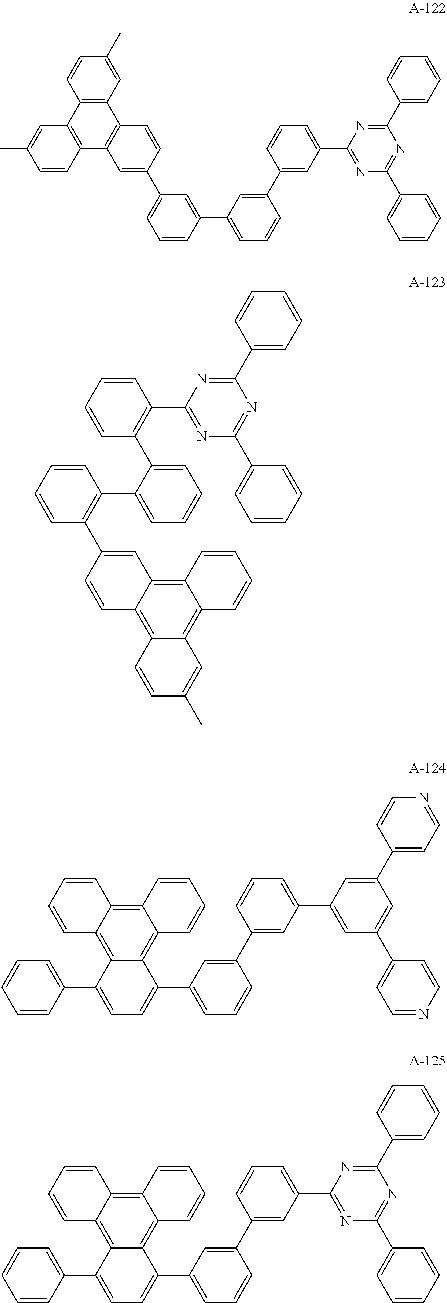 [SMILES: Cc1ccc2c(c1)c1ccc(C)cc1c1ccc(-c3cccc(-c4cccc(-c5cccc(-c6nc(-c7ccccc7)nc(-c7ccccc7)n6)c5)c4)c3)cc21 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2ccc(-c3cccc(-c4cccc(-c5cc(-c6ccncc6)cc(-c6ccncc6)c5)c4)c3)c3c4ccccc4c4ccccc4c23)cc1 ; c1ccc(-c2nc(-c3ccccc3)nc(-c3cccc(-c4cccc(-c5ccc(-c6ccccc6)c6c7ccccc7c7ccccc7c56)c4)c3)n2)cc1]

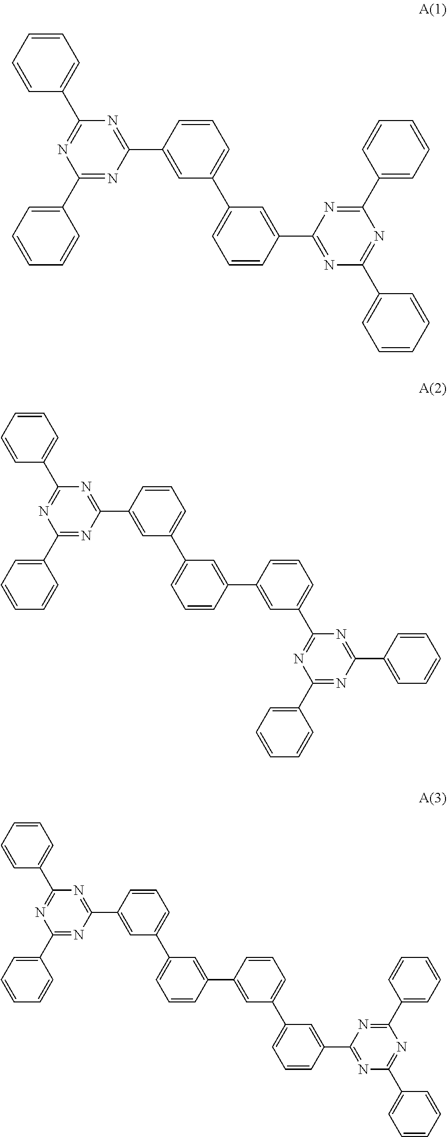 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8)n7)c6)c5)c4)c3)n2)cc1]

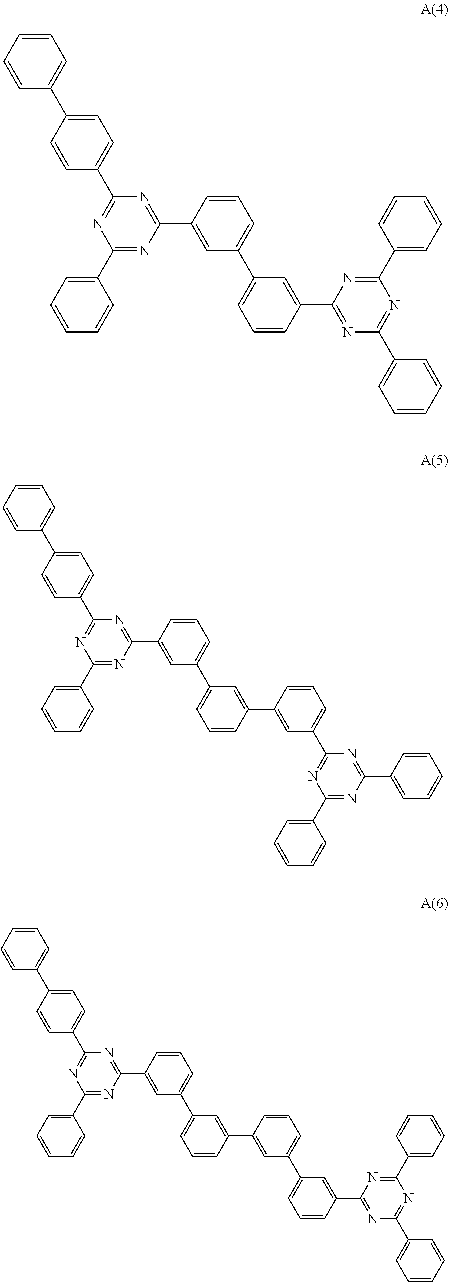 [SMILES: c1ccc(-c2ccc(-c3nc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

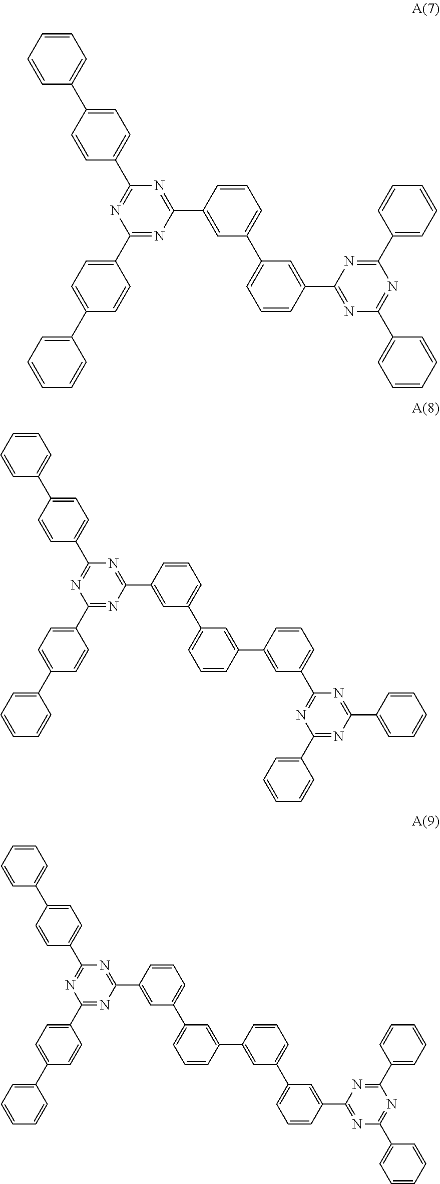 [SMILES: c1ccc(-c2ccc(-c3nc(-c4ccc(-c5ccccc5)cc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccccc9)nc(-c9ccccc9)n8)c7)c6)c5)c4)n3)cc2)cc1]

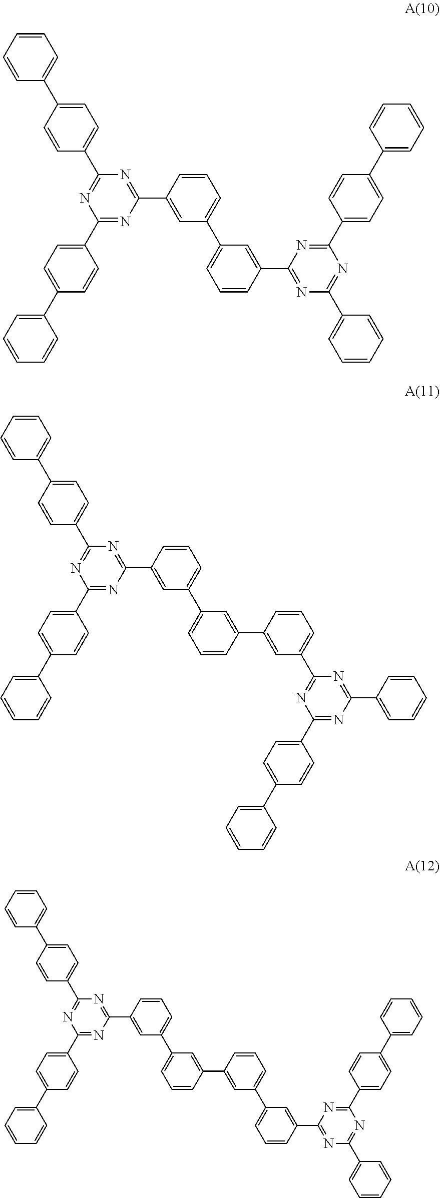 [SMILES: c1ccc(-c2ccc(-c3nc(-c4ccccc4)nc(-c4cccc(-c5cccc(-c6nc(-c7ccc(-c8ccccc8)cc7)nc(-c7ccc(-c8ccccc8)cc7)n6)c5)c4)n3)cc2)cc1 ; c1ccc(-c2ccc(-c3nc(-c4ccccc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

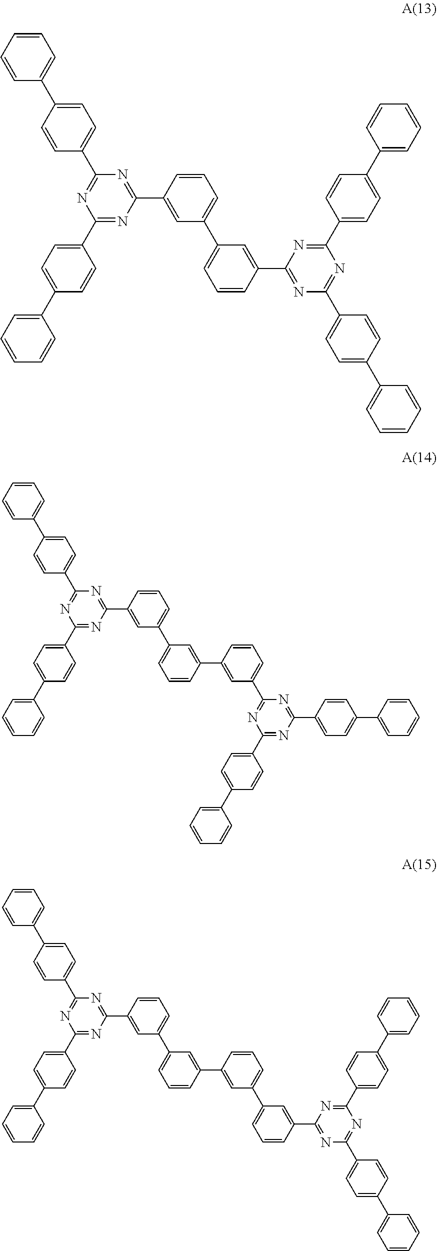 [SMILES: c1ccc(-c2ccc(-c3nc(-c4ccc(-c5ccccc5)cc4)nc(-c4cccc(-c5cccc(-c6nc(-c7ccc(-c8ccccc8)cc7)nc(-c7ccc(-c8ccccc8)cc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

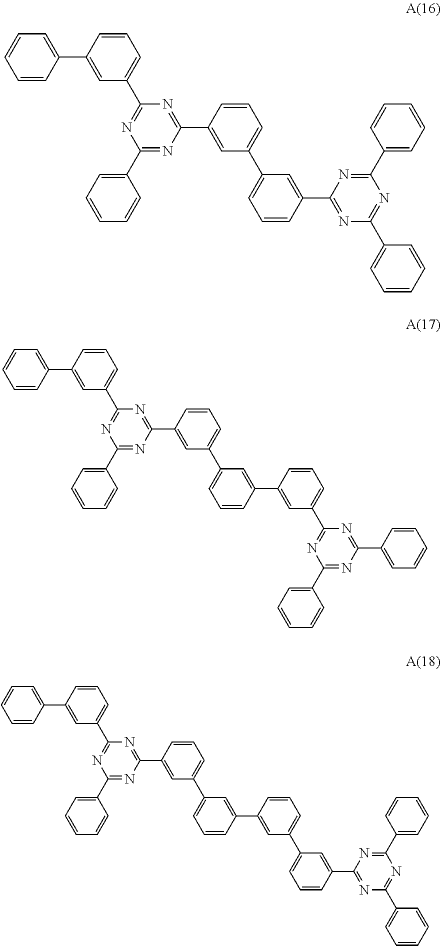 [SMILES: c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

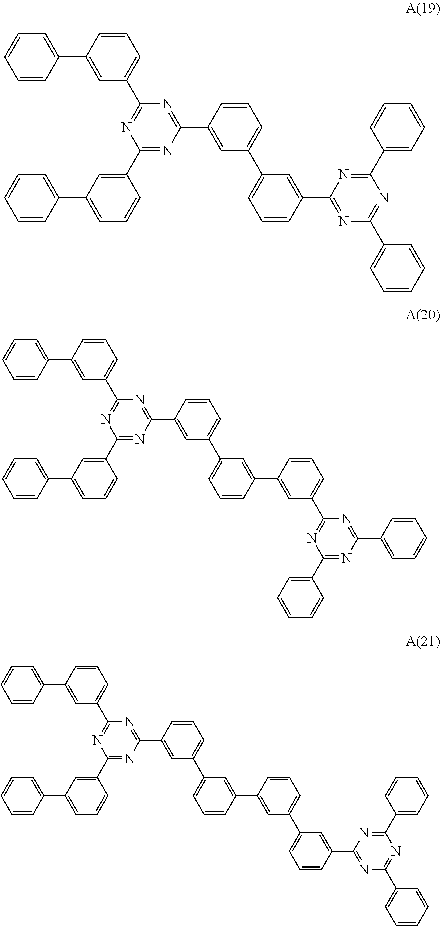 [SMILES: c1ccc(-c2cccc(-c3nc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)c2)cc1]

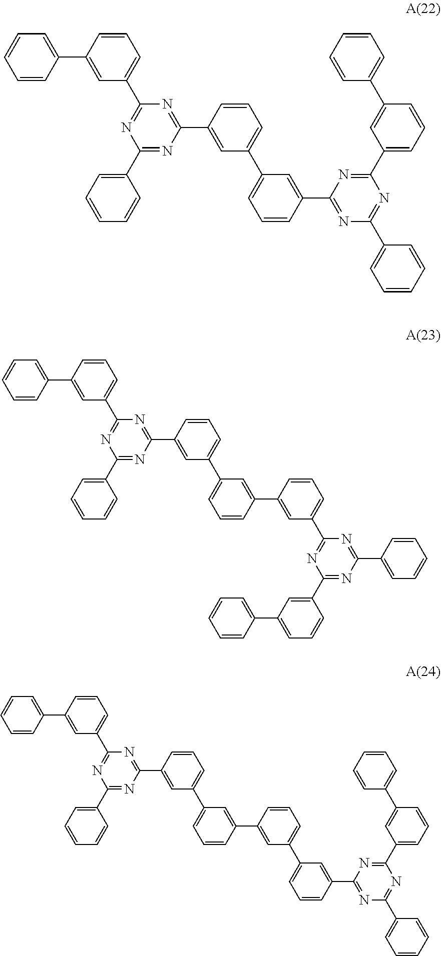 [SMILES: c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)c2)cc1]

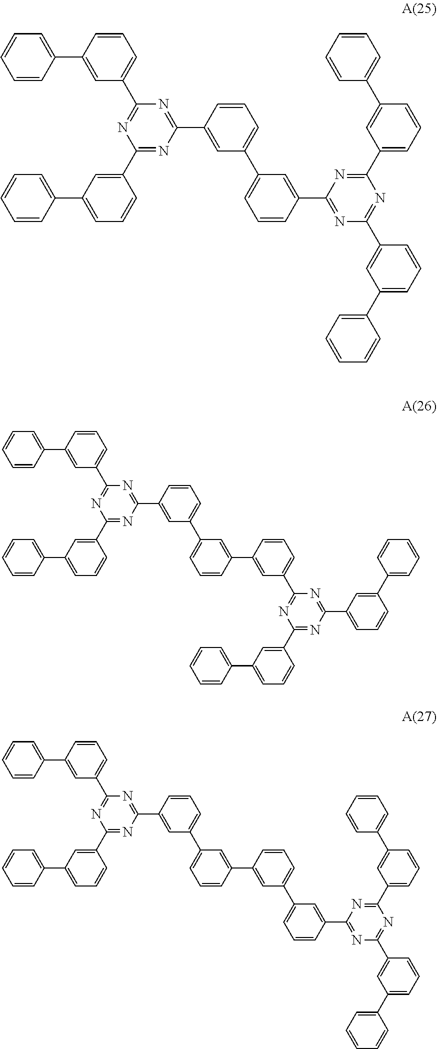 [SMILES: c1ccc(-c2cccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)c2)cc1]

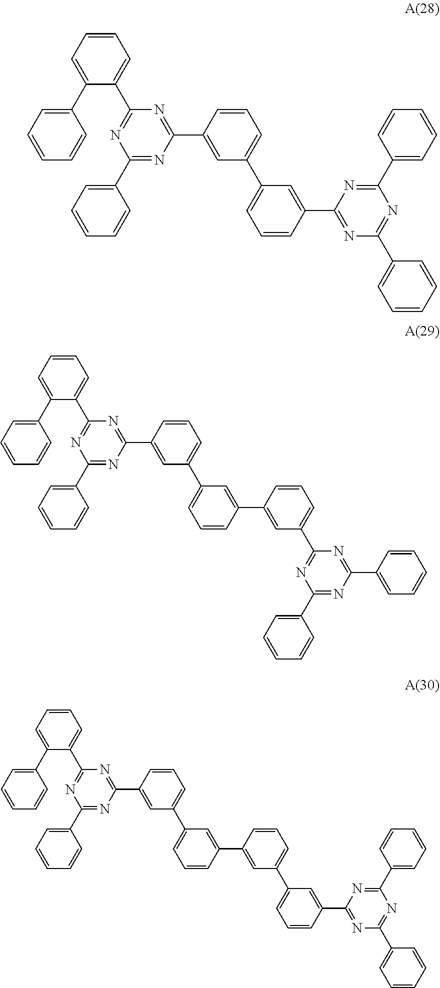 [SMILES: c1ccc(-c2nc(-c3ccccc3)nc(-c3cccc(-c4cccc(-c5nc(-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8-c8ccccc8)n7)c6)c5)c4)c3)n2)cc1]

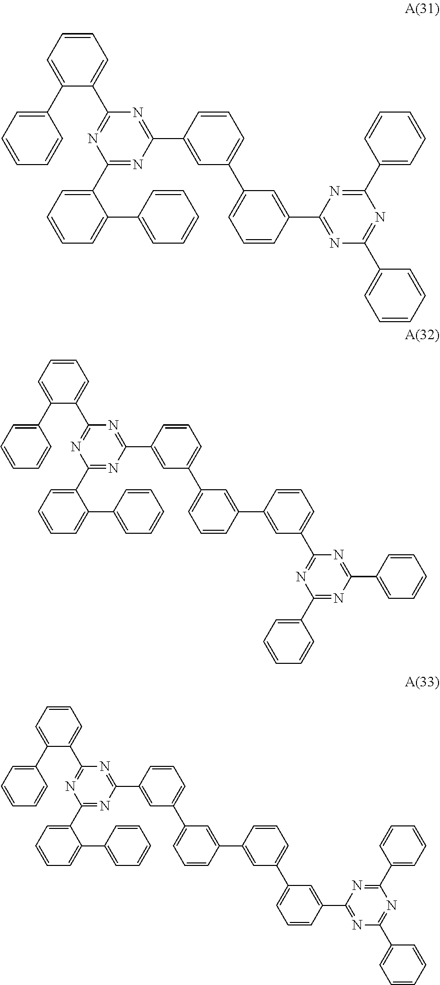 [SMILES: c1ccc(-c2nc(-c3ccccc3)nc(-c3cccc(-c4cccc(-c5nc(-c6ccccc6-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7-c7ccccc7)nc(-c7ccccc7-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8-c8ccccc8)nc(-c8ccccc8-c8ccccc8)n7)c6)c5)c4)c3)n2)cc1]

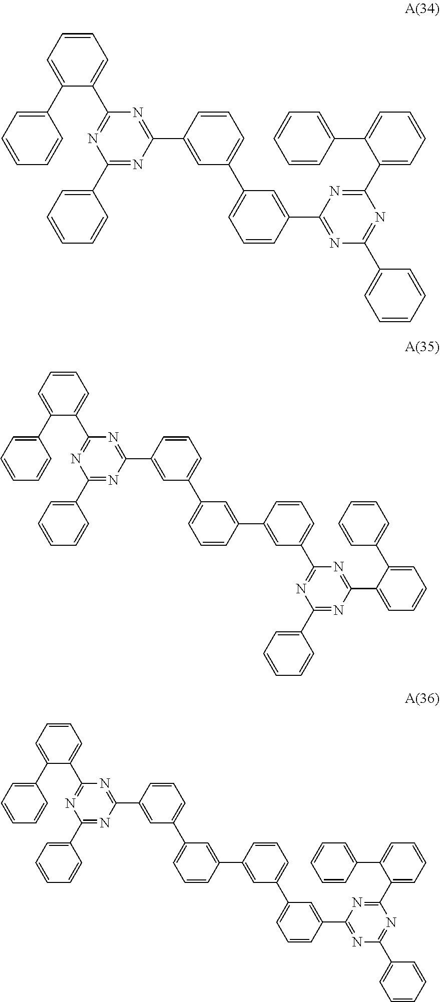 [SMILES: c1ccc(-c2nc(-c3cccc(-c4cccc(-c5nc(-c6ccccc6)nc(-c6ccccc6-c6ccccc6)n5)c4)c3)nc(-c3ccccc3-c3ccccc3)n2)cc1 ; c1ccc(-c2nc(-c3cccc(-c4cccc(-c5cccc(-c6nc(-c7ccccc7)nc(-c7ccccc7-c7ccccc7)n6)c5)c4)c3)nc(-c3ccccc3-c3ccccc3)n2)cc1 ; c1ccc(-c2nc(-c3cccc(-c4cccc(-c5cccc(-c6cccc(-c7nc(-c8ccccc8)nc(-c8ccccc8-c8ccccc8)n7)c6)c5)c4)c3)nc(-c3ccccc3-c3ccccc3)n2)cc1]

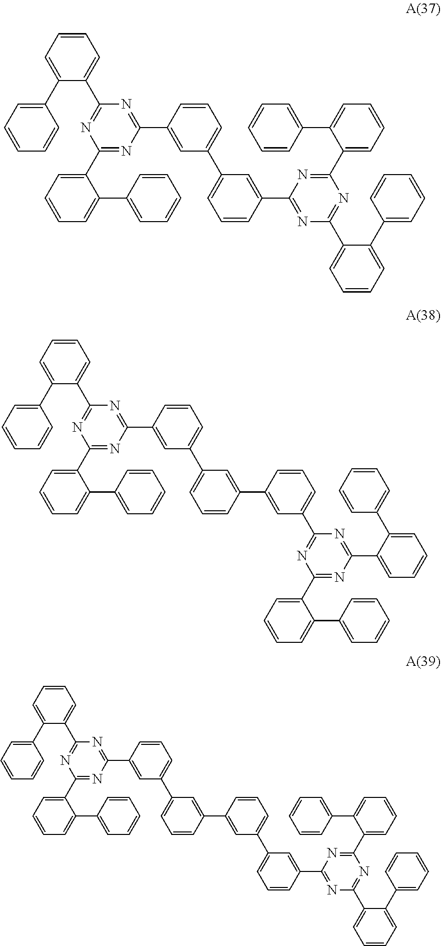 [SMILES: c1ccc(-c2ccccc2-c2nc(-c3cccc(-c4cccc(-c5nc(-c6ccccc6-c6ccccc6)nc(-c6ccccc6-c6ccccc6)n5)c4)c3)nc(-c3ccccc3-c3ccccc3)n2)cc1 ; c1ccc(-c2ccccc2-c2nc(-c3cccc(-c4cccc(-c5cccc(-c6nc(-c7ccccc7-c7ccccc7)nc(-c7ccccc7-c7ccccc7)n6)c5)c4)c3)nc(-c3ccccc3-c3ccccc3)n2)cc1 ; c1ccc(-c2ccccc2-c2nc(-c3cccc(-c4cccc(-c5cccc(-c6cccc(-c7nc(-c8ccccc8-c8ccccc8)nc(-c8ccccc8-c8ccccc8)n7)c6)c5)c4)c3)nc(-c3ccccc3-c3ccccc3)n2)cc1]

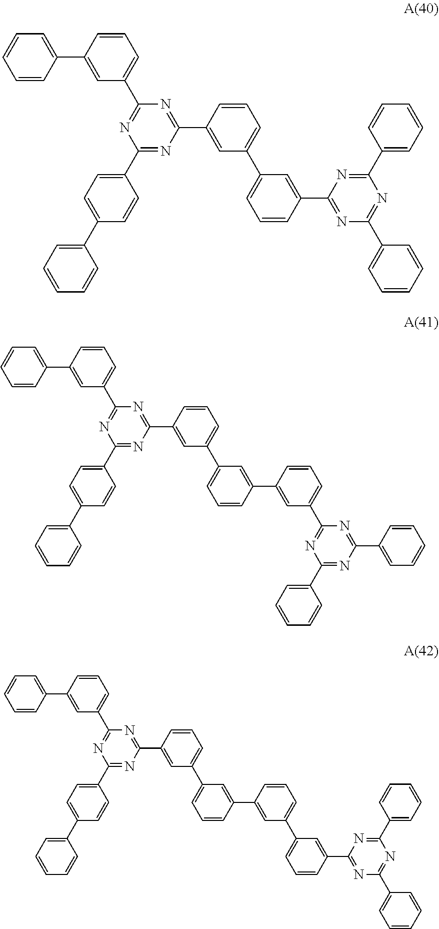 [SMILES: c1ccc(-c2ccc(-c3nc(-c4cccc(-c5ccccc5)c4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)cc2)cc1]

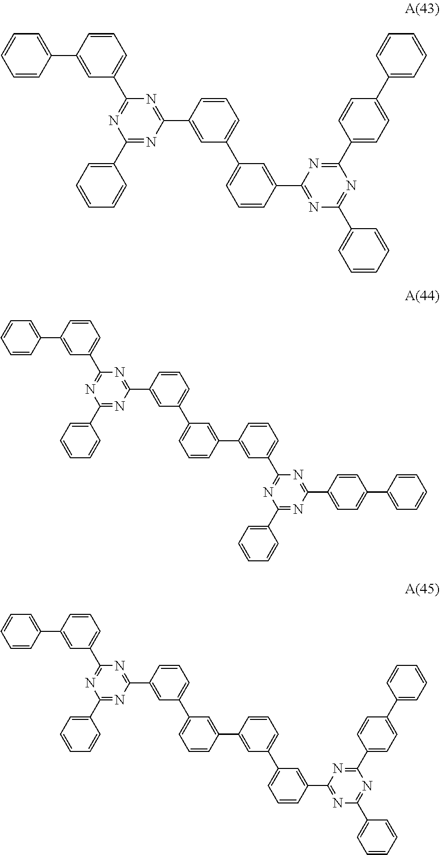 [SMILES: c1ccc(-c2ccc(-c3nc(-c4ccccc4)nc(-c4cccc(-c5cccc(-c6nc(-c7ccccc7)nc(-c7cccc(-c8ccccc8)c7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1]

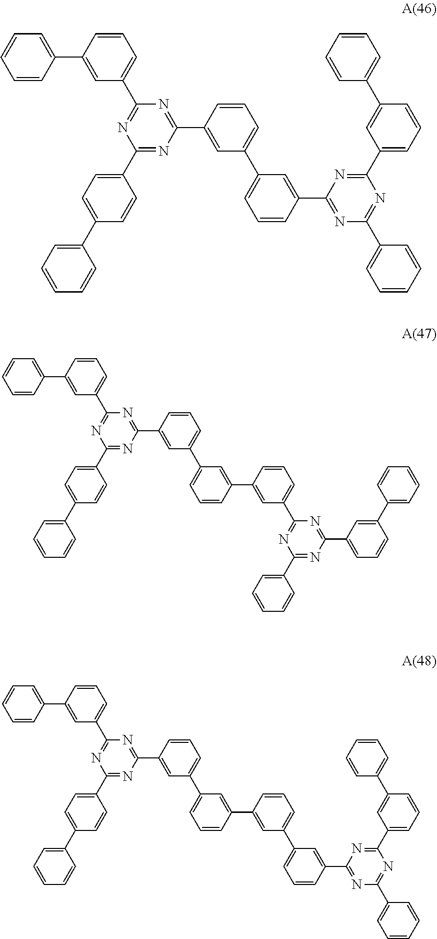 [SMILES: c1ccc(-c2ccc(-c3nc(-c4cccc(-c5ccccc5)c4)nc(-c4cccc(-c5cccc(-c6nc(-c7ccccc7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1]

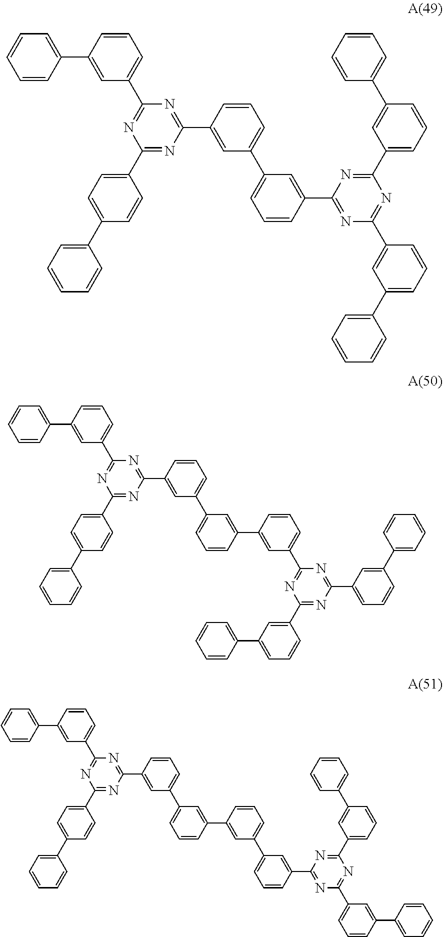 [SMILES: c1ccc(-c2ccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)cc2)cc1]

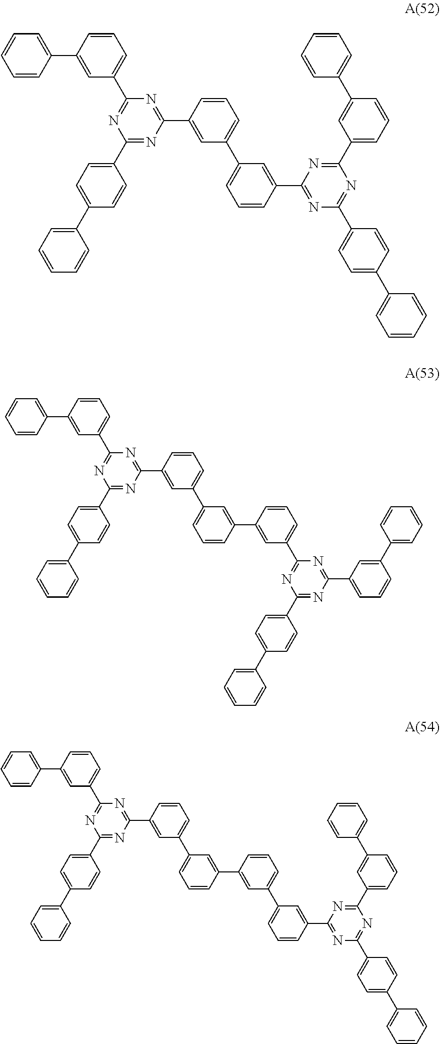 [SMILES: c1ccc(-c2ccc(-c3nc(-c4cccc(-c5ccccc5)c4)nc(-c4cccc(-c5cccc(-c6nc(-c7ccc(-c8ccccc8)cc7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccc(-c9ccccc9)cc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccc(-c%10ccccc%10)cc9)nc(-c9cccc(-c%10ccccc%10)c9)n8)c7)c6)c5)c4)n3)cc2)cc1]

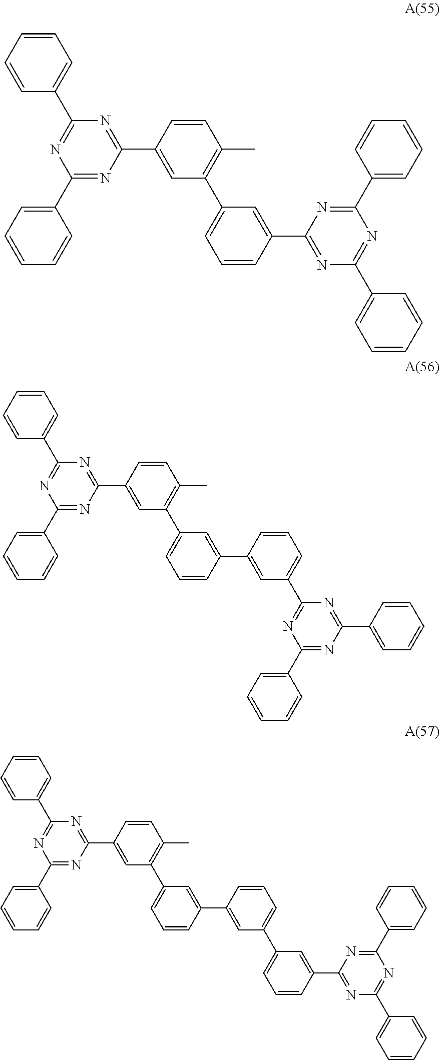 [SMILES: Cc1ccc(-c2nc(-c3ccccc3)nc(-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccccc3)n2)cc1-c1cccc(-c2cccc(-c3cccc(-c4nc(-c5ccccc5)nc(-c5ccccc5)n4)c3)c2)c1]

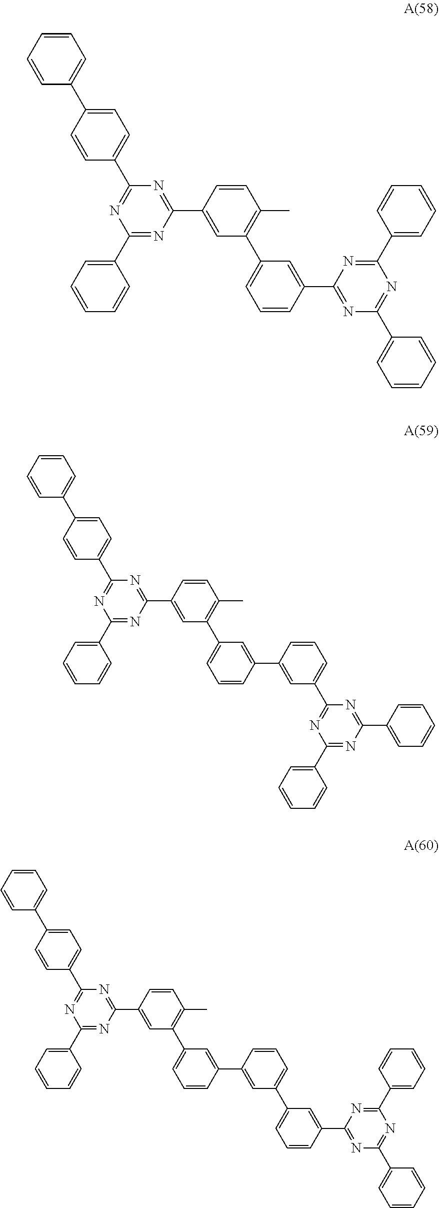 [SMILES: Cc1ccc(-c2nc(-c3ccccc3)nc(-c3ccc(-c4ccccc4)cc3)n2)cc1-c1cccc(-c2nc(-c3ccccc3)nc(-c3ccccc3)n2)c1 ; Cc1ccc(-c2nc(-c3ccccc3)nc(-c3ccc(-c4ccccc4)cc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccc(-c4ccccc4)cc3)n2)cc1-c1cccc(-c2cccc(-c3cccc(-c4nc(-c5ccccc5)nc(-c5ccccc5)n4)c3)c2)c1]

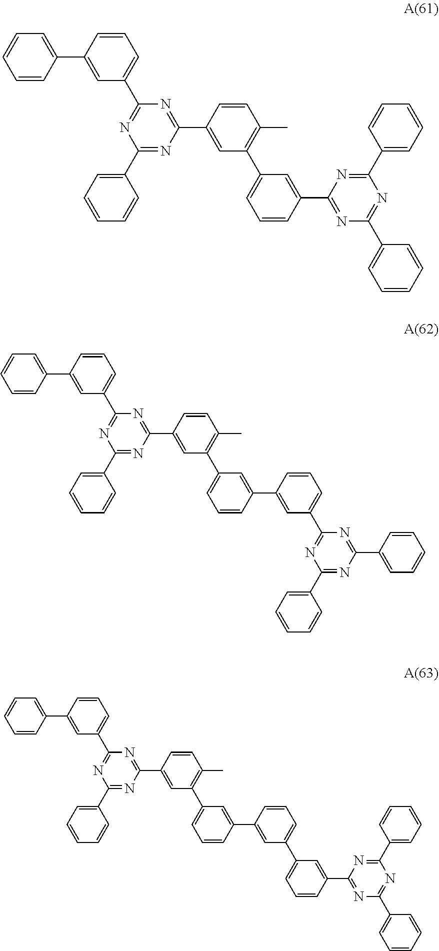 [SMILES: Cc1ccc(-c2nc(-c3ccccc3)nc(-c3cccc(-c4ccccc4)c3)n2)cc1-c1cccc(-c2nc(-c3ccccc3)nc(-c3ccccc3)n2)c1 ; Cc1ccc(-c2nc(-c3ccccc3)nc(-c3cccc(-c4ccccc4)c3)n2)cc1-c1cccc(-c2cccc(-c3nc(-c4ccccc4)nc(-c4ccccc4)n3)c2)c1 ; Cc1ccc(-c2nc(-c3ccccc3)nc(-c3cccc(-c4ccccc4)c3)n2)cc1-c1cccc(-c2cccc(-c3cccc(-c4nc(-c5ccccc5)nc(-c5ccccc5)n4)c3)c2)c1]

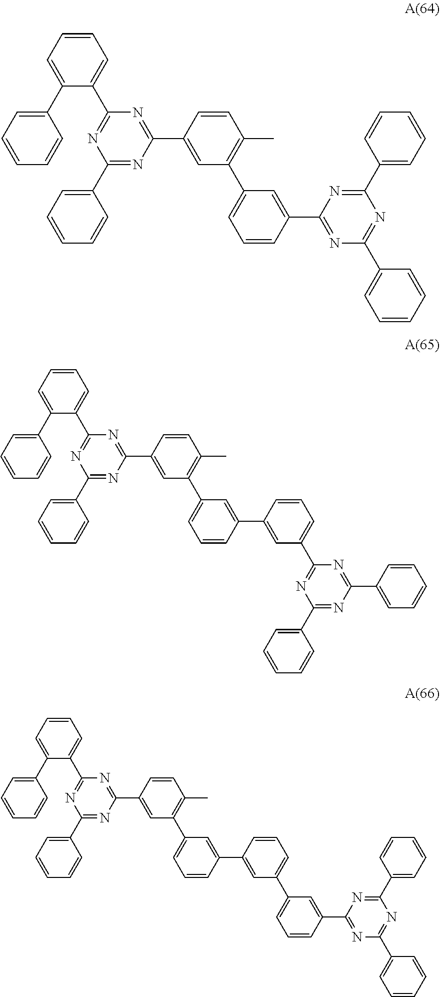 [SMILES: Cc1ccc(-c2nc(-c3ccccc3)nc(-c3ccccc3-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3-c3ccccc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccccc3-c3ccccc3)n2)cc1-c1cccc(-c2cccc(-c3cccc(-c4nc(-c5ccccc5)nc(-c5ccccc5)n4)c3)c2)c1]

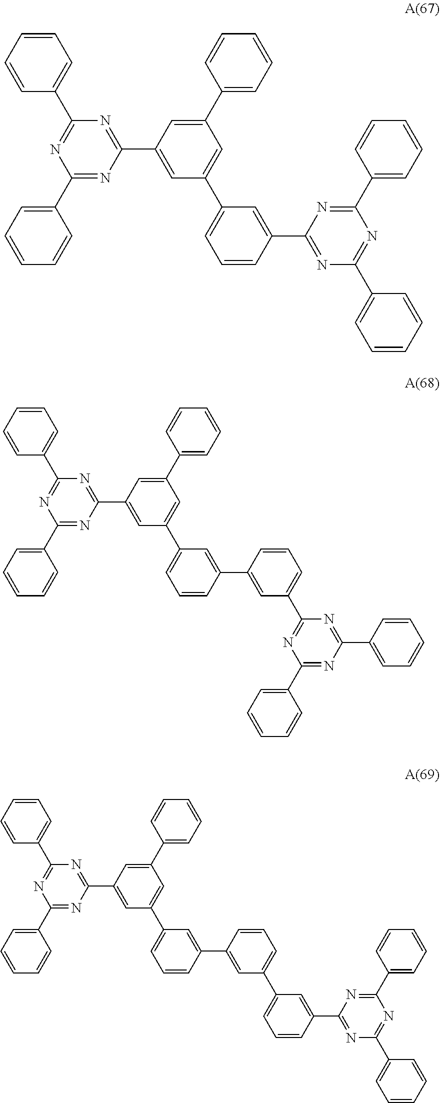 [SMILES: c1ccc(-c2cc(-c3cccc(-c4nc(-c5ccccc5)nc(-c5ccccc5)n4)c3)cc(-c3nc(-c4ccccc4)nc(-c4ccccc4)n3)c2)cc1 ; c1ccc(-c2cc(-c3cccc(-c4cccc(-c5nc(-c6ccccc6)nc(-c6ccccc6)n5)c4)c3)cc(-c3nc(-c4ccccc4)nc(-c4ccccc4)n3)c2)cc1 ; c1ccc(-c2cc(-c3cccc(-c4cccc(-c5cccc(-c6nc(-c7ccccc7)nc(-c7ccccc7)n6)c5)c4)c3)cc(-c3nc(-c4ccccc4)nc(-c4ccccc4)n3)c2)cc1]

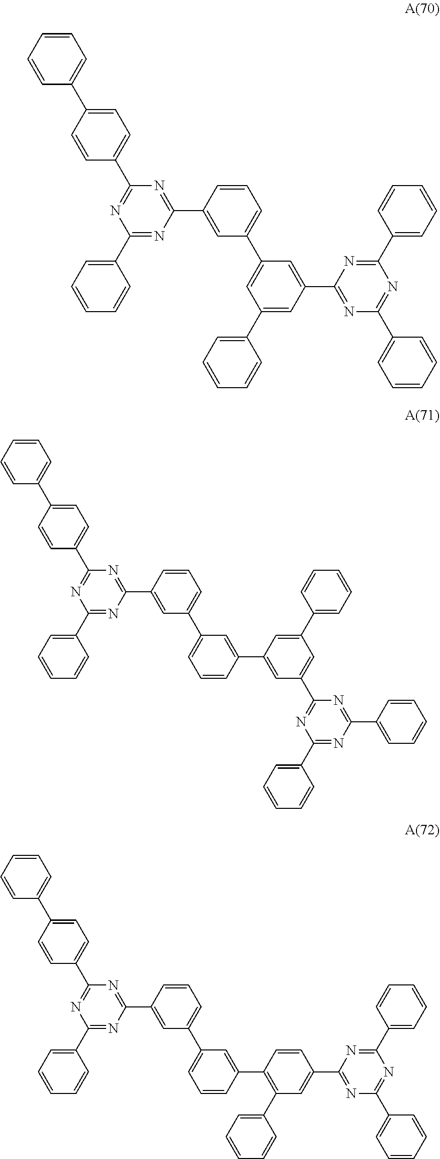 [SMILES: c1ccc(-c2ccc(-c3nc(-c4ccccc4)nc(-c4cccc(-c5cc(-c6ccccc6)cc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cc(-c7ccccc7)cc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6ccc(-c7nc(-c8ccccc8)nc(-c8ccccc8)n7)cc6-c6ccccc6)c5)c4)n3)cc2)cc1]

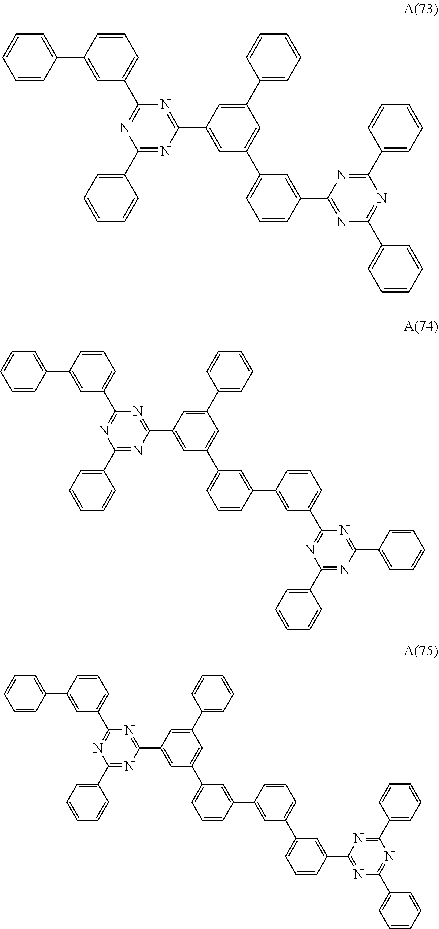 [SMILES: c1ccc(-c2cccc(-c3nc(-c4ccccc4)nc(-c4cc(-c5ccccc5)cc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cc(-c5ccccc5)cc(-c5cccc(-c6cccc(-c7nc(-c8ccccc8)nc(-c8ccccc8)n7)c6)c5)c4)n3)c2)cc1 ; c1ccc(-c2cccc(-c3nc(-c4ccccc4)nc(-c4cc(-c5ccccc5)cc(-c5cccc(-c6cccc(-c7cccc(-c8nc(-c9ccccc9)nc(-c9ccccc9)n8)c7)c6)c5)c4)n3)c2)cc1]

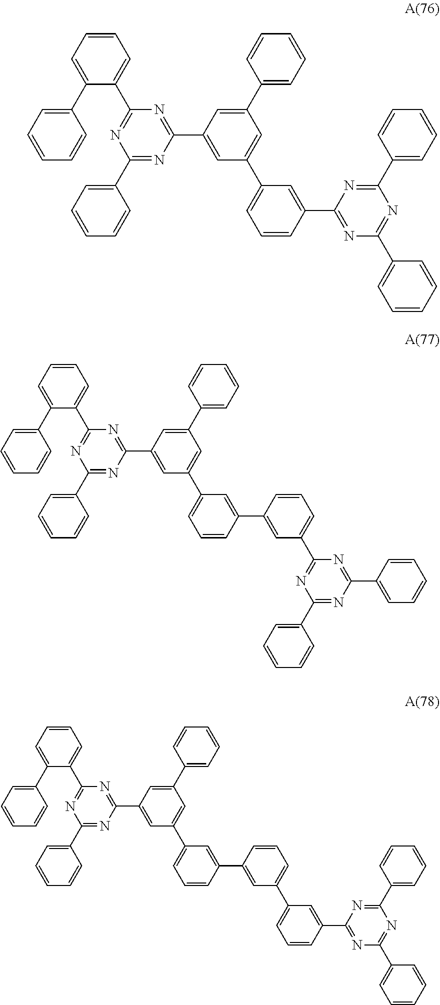 [SMILES: c1ccc(-c2cc(-c3cccc(-c4nc(-c5ccccc5)nc(-c5ccccc5)n4)c3)cc(-c3nc(-c4ccccc4)nc(-c4ccccc4-c4ccccc4)n3)c2)cc1 ; c1ccc(-c2cc(-c3cccc(-c4cccc(-c5nc(-c6ccccc6)nc(-c6ccccc6)n5)c4)c3)cc(-c3nc(-c4ccccc4)nc(-c4ccccc4-c4ccccc4)n3)c2)cc1 ; c1ccc(-c2cc(-c3cccc(-c4cccc(-c5cccc(-c6nc(-c7ccccc7)nc(-c7ccccc7)n6)c5)c4)c3)cc(-c3nc(-c4ccccc4)nc(-c4ccccc4-c4ccccc4)n3)c2)cc1]

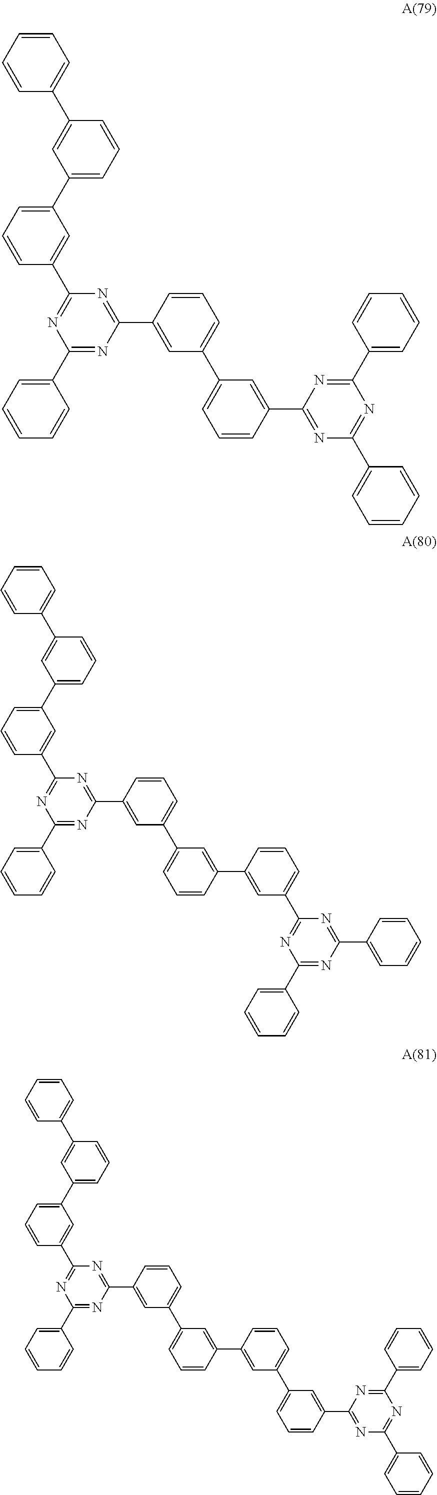 [SMILES: c1ccc(-c2cccc(-c3cccc(-c4nc(-c5ccccc5)nc(-c5cccc(-c6cccc(-c7nc(-c8ccccc8)nc(-c8ccccc8)n7)c6)c5)n4)c3)c2)cc1 ; c1ccc(-c2cccc(-c3cccc(-c4nc(-c5ccccc5)nc(-c5cccc(-c6cccc(-c7cccc(-c8nc(-c9ccccc9)nc(-c9ccccc9)n8)c7)c6)c5)n4)c3)c2)cc1 ; c1ccc(-c2cccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)c2)cc1]

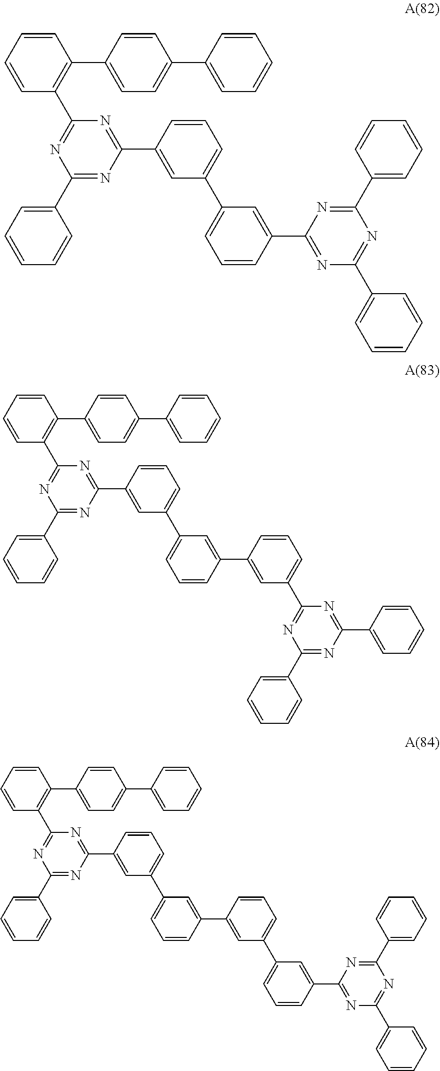 [SMILES: c1ccc(-c2ccc(-c3ccccc3-c3nc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3ccccc3-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

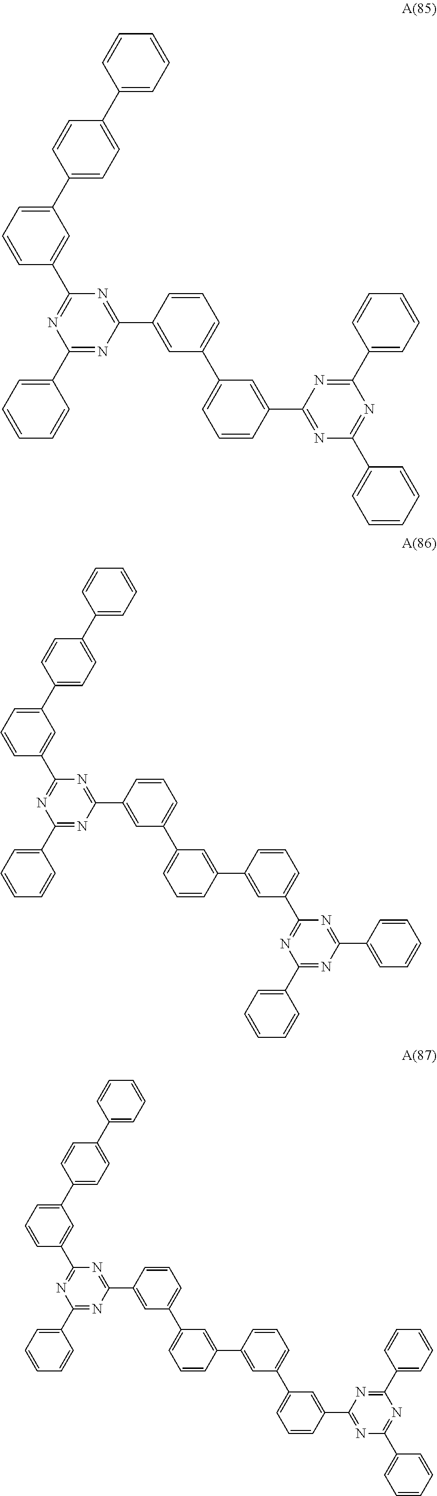 [SMILES: c1ccc(-c2ccc(-c3cccc(-c4nc(-c5ccccc5)nc(-c5cccc(-c6cccc(-c7nc(-c8ccccc8)nc(-c8ccccc8)n7)c6)c5)n4)c3)cc2)cc1 ; c1ccc(-c2ccc(-c3cccc(-c4nc(-c5ccccc5)nc(-c5cccc(-c6cccc(-c7cccc(-c8nc(-c9ccccc9)nc(-c9ccccc9)n8)c7)c6)c5)n4)c3)cc2)cc1 ; c1ccc(-c2ccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)cc2)cc1]

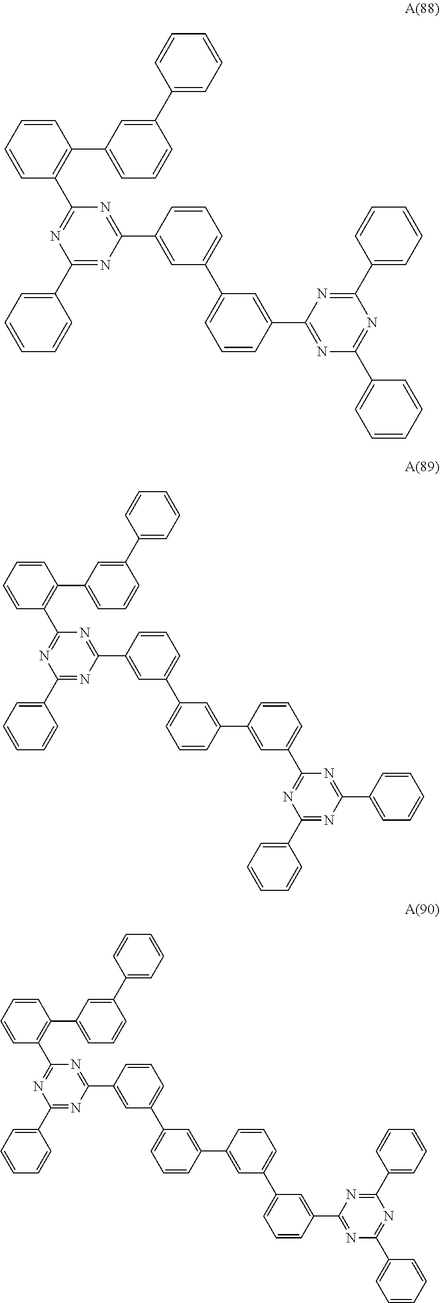 [SMILES: c1ccc(-c2cccc(-c3ccccc3-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3ccccc3-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

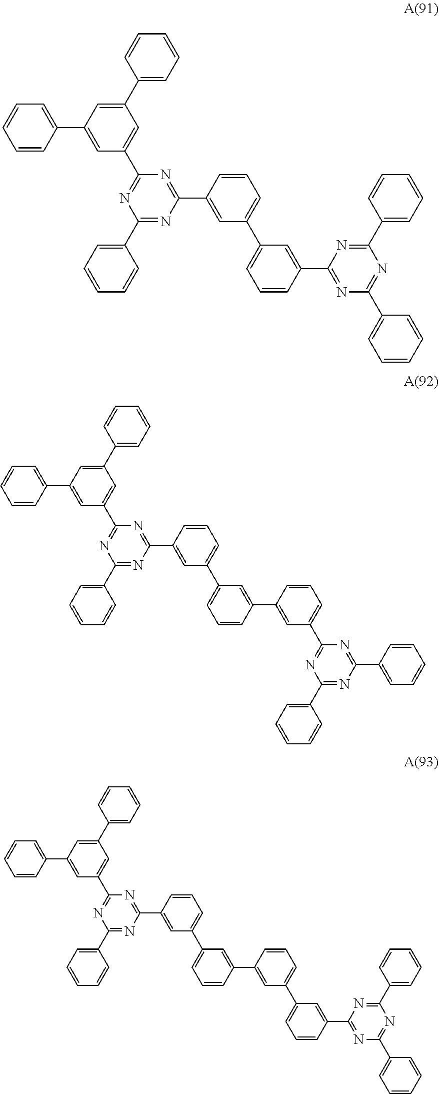 [SMILES: c1ccc(-c2cc(-c3ccccc3)cc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cc(-c3ccccc3)cc(-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cc(-c3ccccc3)cc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

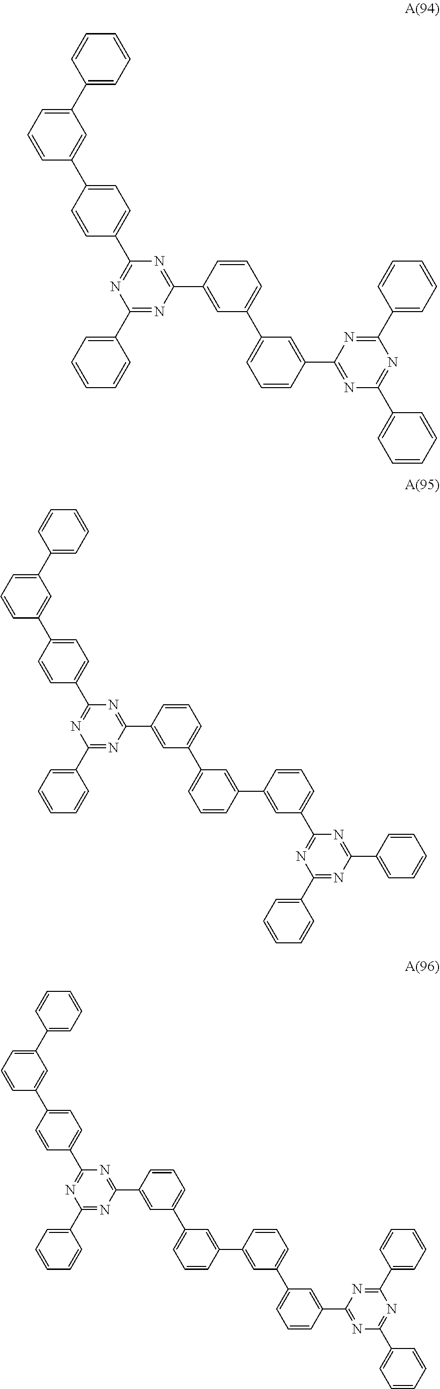 [SMILES: c1ccc(-c2cccc(-c3ccc(-c4nc(-c5ccccc5)nc(-c5cccc(-c6cccc(-c7nc(-c8ccccc8)nc(-c8ccccc8)n7)c6)c5)n4)cc3)c2)cc1 ; c1ccc(-c2cccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)c2)cc1 ; c1ccc(-c2cccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)c2)cc1]

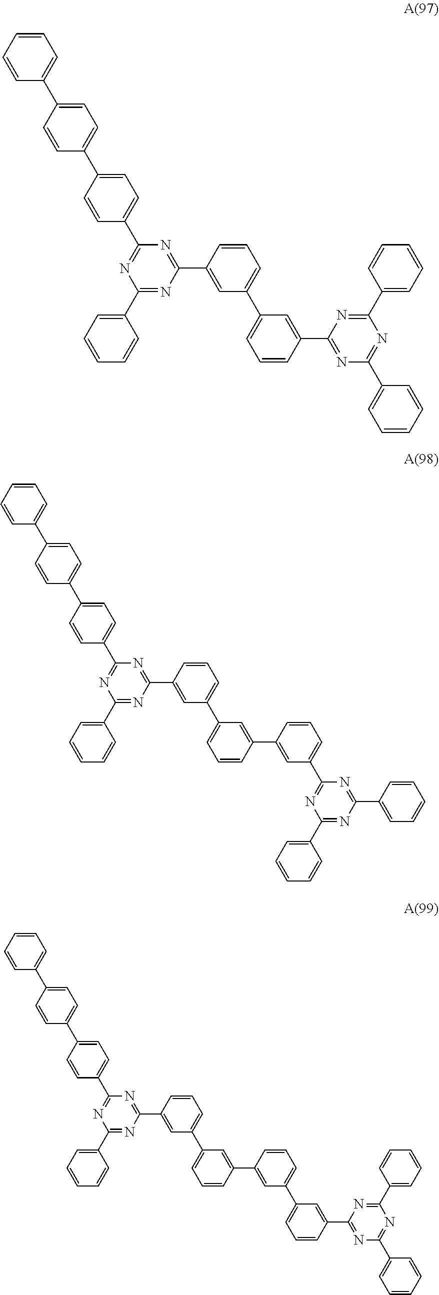 [SMILES: c1ccc(-c2ccc(-c3ccc(-c4nc(-c5ccccc5)nc(-c5cccc(-c6cccc(-c7nc(-c8ccccc8)nc(-c8ccccc8)n7)c6)c5)n4)cc3)cc2)cc1 ; c1ccc(-c2ccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)cc2)cc1 ; c1ccc(-c2ccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)cc2)cc1]

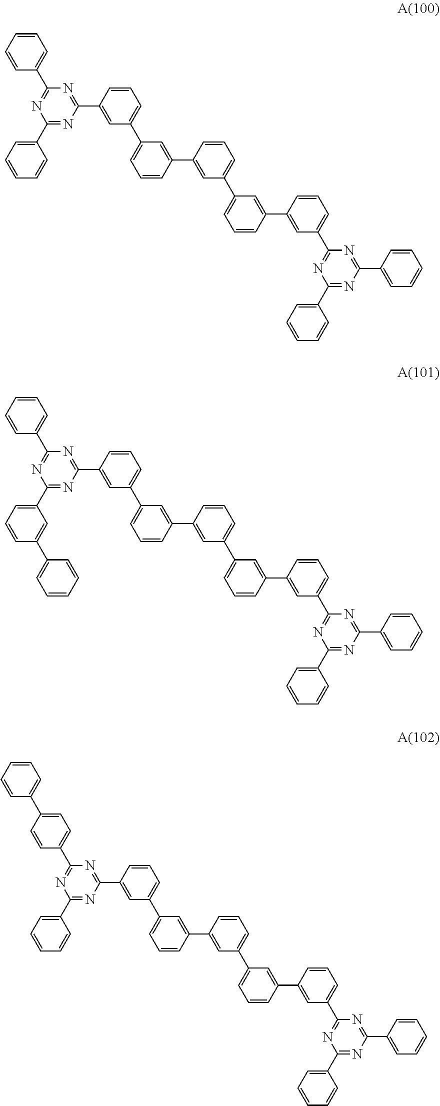 [SMILES: c1ccc(-c2nc(-c3ccccc3)nc(-c3cccc(-c4cccc(-c5cccc(-c6cccc(-c7cccc(-c8nc(-c9ccccc9)nc(-c9ccccc9)n8)c7)c6)c5)c4)c3)n2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1 ; c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1]

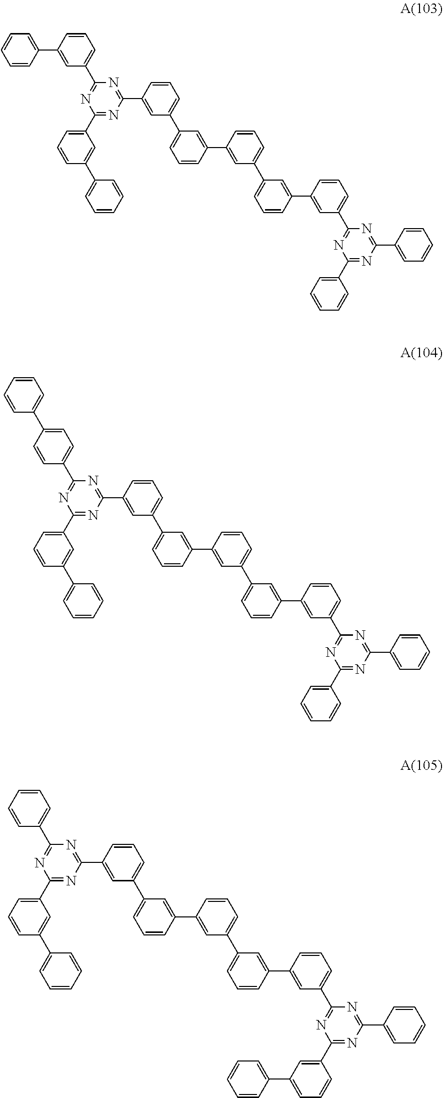 [SMILES: c1ccc(-c2cccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1 ; c1ccc(-c2ccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)c2)cc1]

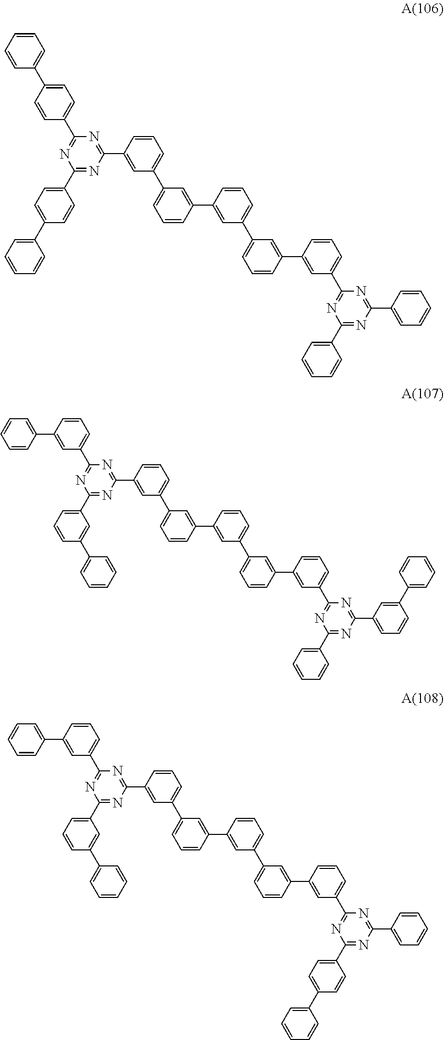 [SMILES: c1ccc(-c2ccc(-c3nc(-c4ccc(-c5ccccc5)cc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1 ; c1ccc(-c2ccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)cc2)cc1]

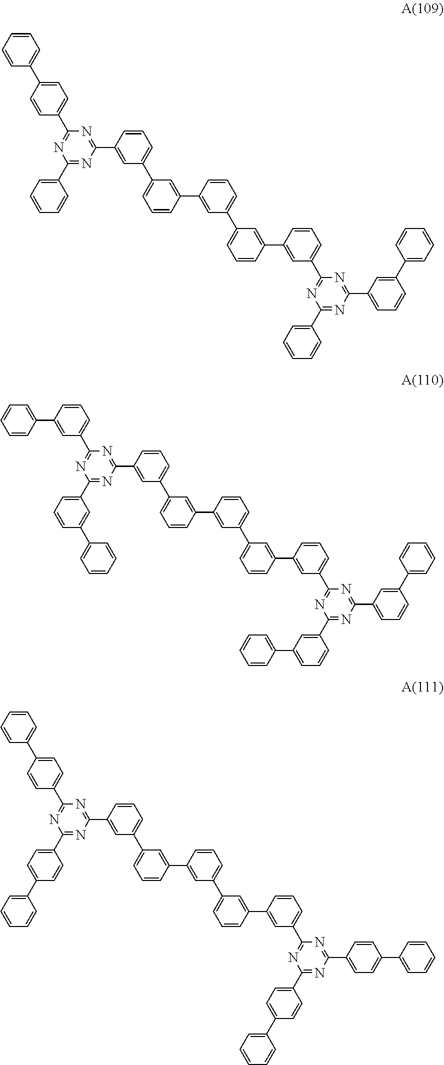

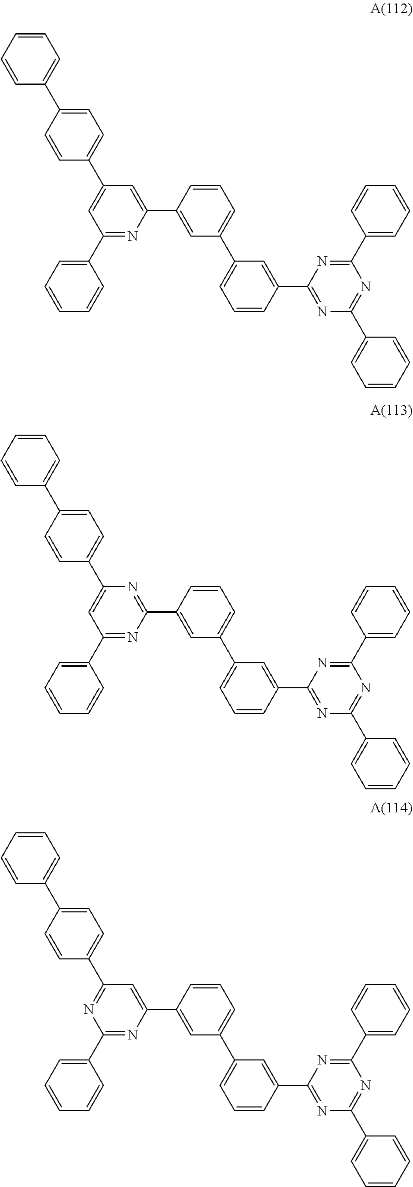 [SMILES: c1ccc(-c2ccc(-c3cc(-c4ccccc4)nc(-c4cccc(-c5cccc(-c6nc(-c7ccccc7)nc(-c7ccccc7)n6)c5)c4)c3)cc2)cc1 ; c1ccc(-c2ccc(-c3cc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3cc(-c4cccc(-c5cccc(-c6nc(-c7ccccc7)nc(-c7ccccc7)n6)c5)c4)nc(-c4ccccc4)n3)cc2)cc1]

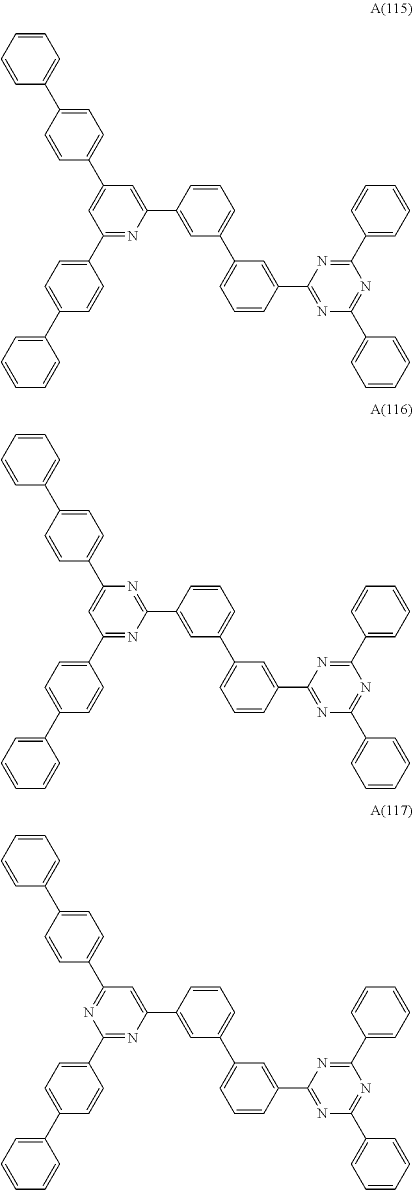 [SMILES: c1ccc(-c2ccc(-c3cc(-c4ccc(-c5ccccc5)cc4)nc(-c4cccc(-c5cccc(-c6nc(-c7ccccc7)nc(-c7ccccc7)n6)c5)c4)c3)cc2)cc1 ; c1ccc(-c2ccc(-c3cc(-c4ccc(-c5ccccc5)cc4)nc(-c4cccc(-c5cccc(-c6nc(-c7ccccc7)nc(-c7ccccc7)n6)c5)c4)n3)cc2)cc1 ; c1ccc(-c2ccc(-c3cc(-c4cccc(-c5cccc(-c6nc(-c7ccccc7)nc(-c7ccccc7)n6)c5)c4)nc(-c4ccc(-c5ccccc5)cc4)n3)cc2)cc1]

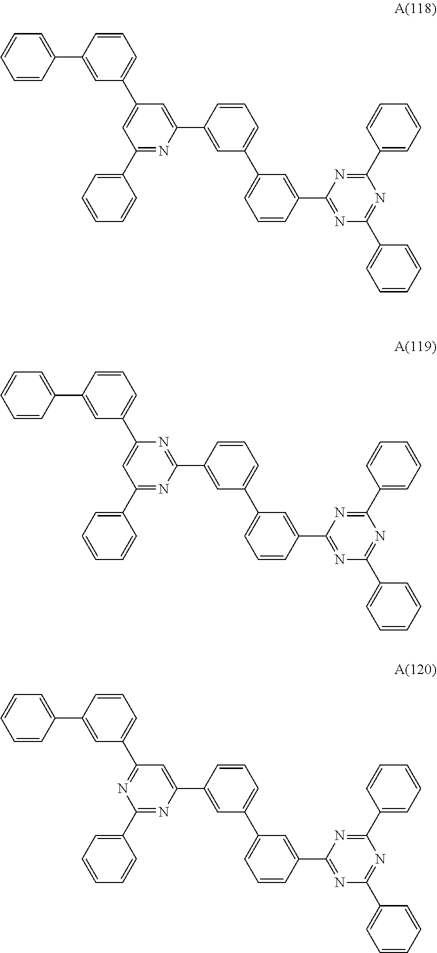 [SMILES: c1ccc(-c2cccc(-c3cc(-c4ccccc4)nc(-c4cccc(-c5cccc(-c6nc(-c7ccccc7)nc(-c7ccccc7)n6)c5)c4)c3)c2)cc1 ; c1ccc(-c2cccc(-c3cc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3cc(-c4cccc(-c5cccc(-c6nc(-c7ccccc7)nc(-c7ccccc7)n6)c5)c4)nc(-c4ccccc4)n3)c2)cc1]

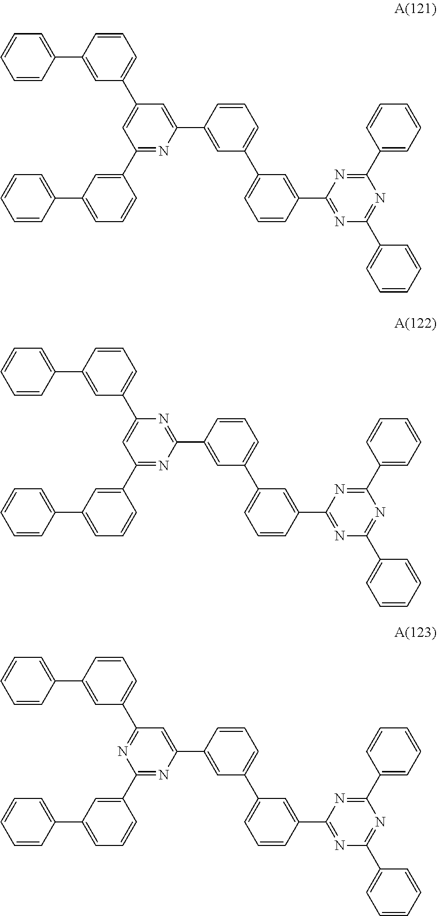 [SMILES: c1ccc(-c2cccc(-c3cc(-c4cccc(-c5ccccc5)c4)nc(-c4cccc(-c5cccc(-c6nc(-c7ccccc7)nc(-c7ccccc7)n6)c5)c4)c3)c2)cc1 ; c1ccc(-c2cccc(-c3cc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3cc(-c4cccc(-c5cccc(-c6nc(-c7ccccc7)nc(-c7ccccc7)n6)c5)c4)nc(-c4cccc(-c5ccccc5)c4)n3)c2)cc1]

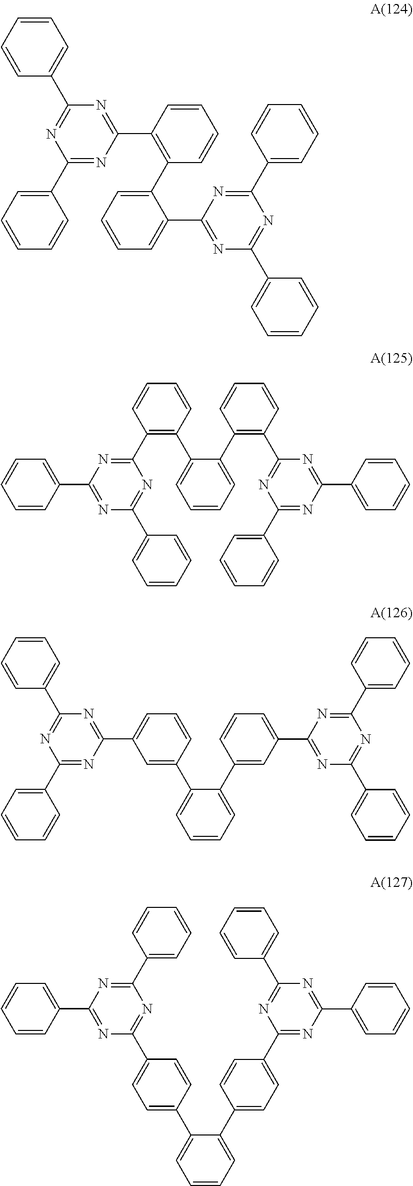 [SMILES: c1ccc(-c2nc(-c3ccccc3)nc(-c3ccccc3-c3ccccc3-c3nc(-c4ccccc4)nc(-c4ccccc4)n3)n2)cc1 ; c1ccc(-c2nc(-c3ccccc3)nc(-c3ccccc3-c3ccccc3-c3ccccc3-c3nc(-c4ccccc4)nc(-c4ccccc4)n3)n2)cc1 ; c1ccc(-c2nc(-c3ccccc3)nc(-c3cccc(-c4ccccc4-c4cccc(-c5nc(-c6ccccc6)nc(-c6ccccc6)n5)c4)c3)n2)cc1 ; c1ccc(-c2nc(-c3ccccc3)nc(-c3ccc(-c4ccccc4-c4ccc(-c5nc(-c6ccccc6)nc(-c6ccccc6)n5)cc4)cc3)n2)cc1]

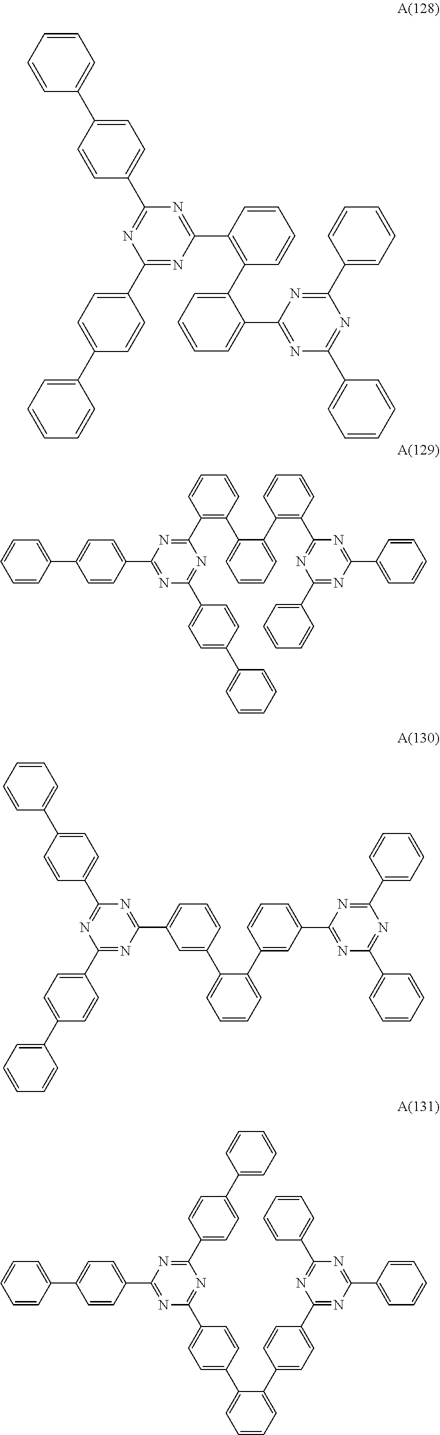 [SMILES: c1ccc(-c2ccc(-c3nc(-c4ccc(-c5ccccc5)cc4)nc(-c4ccccc4-c4ccccc4-c4nc(-c5ccccc5)nc(-c5ccccc5)n4)n3)cc2)cc1 ; c1ccc(-c2ccc(-c3nc(-c4ccc(-c5ccccc5)cc4)nc(-c4ccccc4-c4ccccc4-c4ccccc4-c4nc(-c5ccccc5)nc(-c5ccccc5)n4)n3)cc2)cc1 ; c1ccc(-c2ccc(-c3nc(-c4ccc(-c5ccccc5)cc4)nc(-c4cccc(-c5ccccc5-c5cccc(-c6nc(-c7ccccc7)nc(-c7ccccc7)n6)c5)c4)n3)cc2)cc1 ; c1ccc(-c2ccc(-c3nc(-c4ccc(-c5ccccc5)cc4)nc(-c4ccc(-c5ccccc5-c5ccc(-c6nc(-c7ccccc7)nc(-c7ccccc7)n6)cc5)cc4)n3)cc2)cc1]

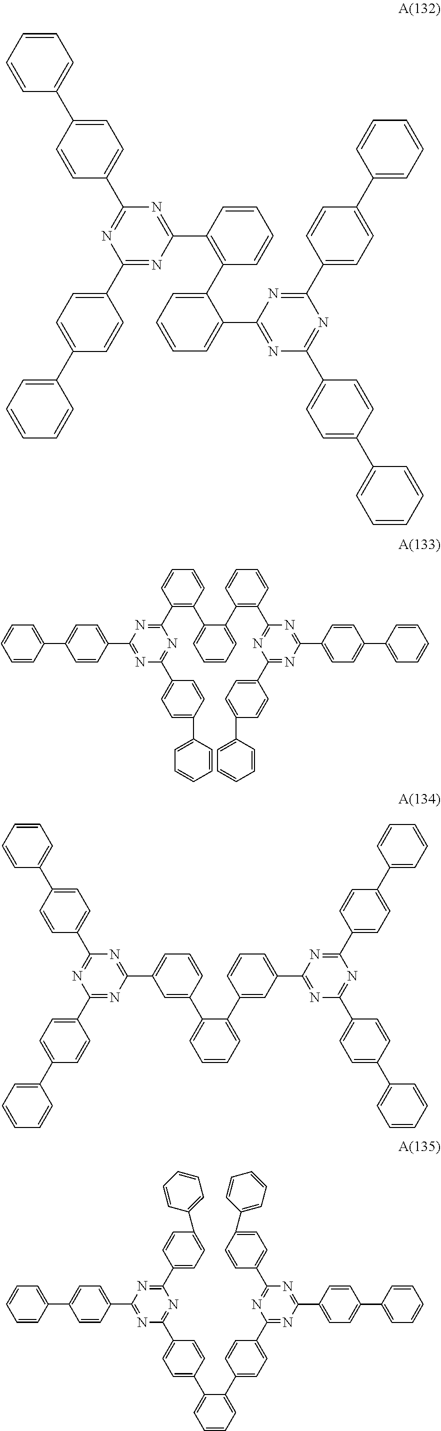

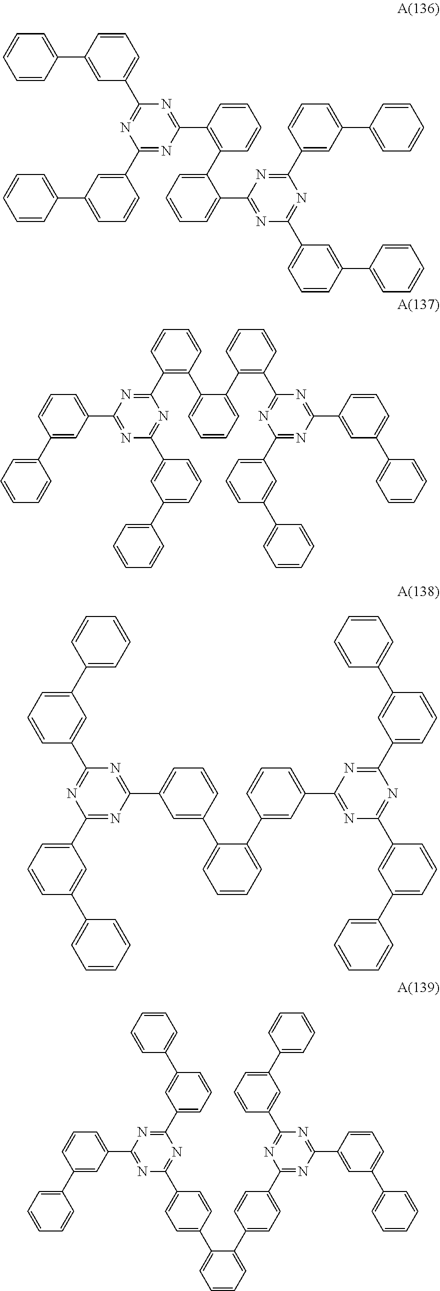

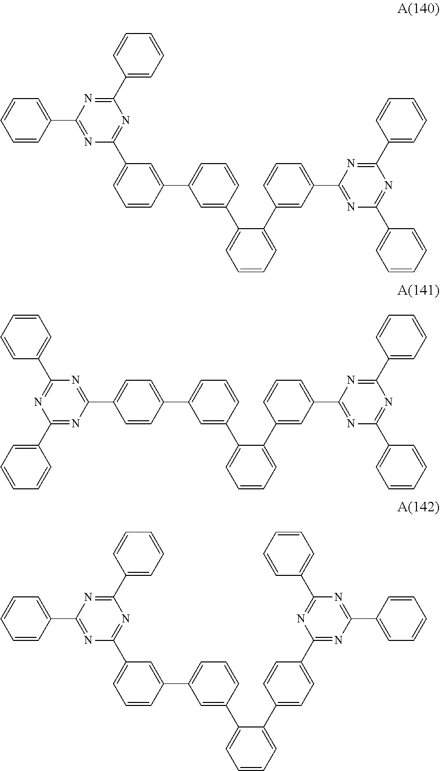 [SMILES: c1ccc(-c2nc(-c3ccccc3)nc(-c3cccc(-c4cccc(-c5ccccc5-c5cccc(-c6nc(-c7ccccc7)nc(-c7ccccc7)n6)c5)c4)c3)n2)cc1 ; c1ccc(-c2nc(-c3ccccc3)nc(-c3ccc(-c4cccc(-c5ccccc5-c5cccc(-c6nc(-c7ccccc7)nc(-c7ccccc7)n6)c5)c4)cc3)n2)cc1 ; c1ccc(-c2nc(-c3ccccc3)nc(-c3ccc(-c4ccccc4-c4cccc(-c5cccc(-c6nc(-c7ccccc7)nc(-c7ccccc7)n6)c5)c4)cc3)n2)cc1]

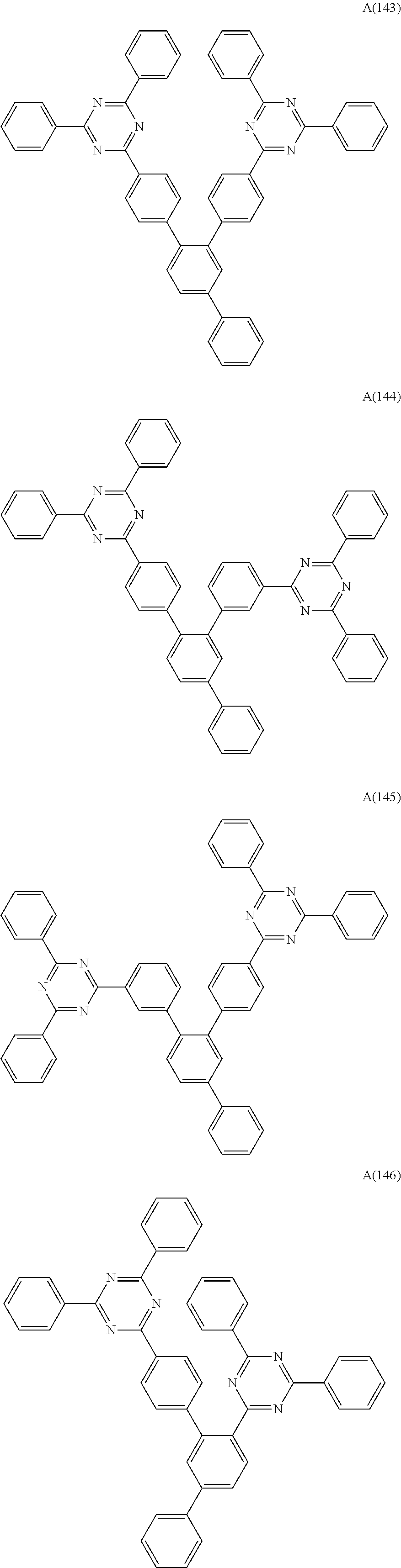 [SMILES: c1ccc(-c2ccc(-c3ccc(-c4nc(-c5ccccc5)nc(-c5ccccc5)n4)cc3)c(-c3ccc(-c4nc(-c5ccccc5)nc(-c5ccccc5)n4)cc3)c2)cc1 ; c1ccc(-c2ccc(-c3ccc(-c4nc(-c5ccccc5)nc(-c5ccccc5)n4)cc3)c(-c3cccc(-c4nc(-c5ccccc5)nc(-c5ccccc5)n4)c3)c2)cc1 ; c1ccc(-c2ccc(-c3cccc(-c4nc(-c5ccccc5)nc(-c5ccccc5)n4)c3)c(-c3ccc(-c4nc(-c5ccccc5)nc(-c5ccccc5)n4)cc3)c2)cc1 ; c1ccc(-c2ccc(-c3nc(-c4ccccc4)nc(-c4ccccc4)n3)c(-c3ccc(-c4nc(-c5ccccc5)nc(-c5ccccc5)n4)cc3)c2)cc1]

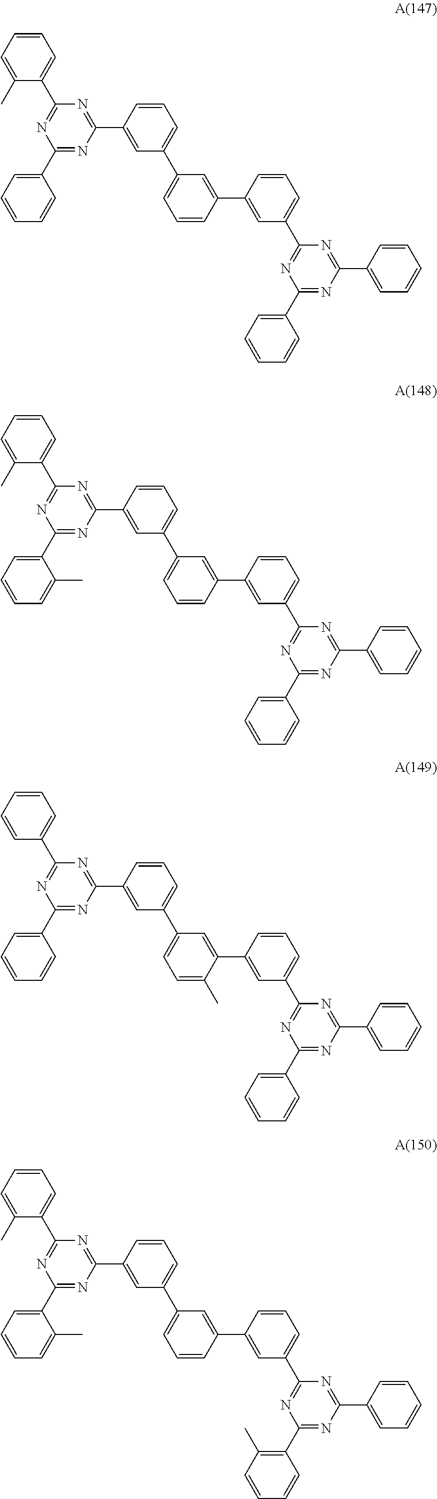 [SMILES: Cc1ccccc1-c1nc(-c2ccccc2)nc(-c2cccc(-c3cccc(-c4cccc(-c5nc(-c6ccccc6)nc(-c6ccccc6)n5)c4)c3)c2)n1 ; Cc1ccccc1-c1nc(-c2cccc(-c3cccc(-c4cccc(-c5nc(-c6ccccc6)nc(-c6ccccc6)n5)c4)c3)c2)nc(-c2ccccc2C)n1 ; Cc1ccc(-c2cccc(-c3nc(-c4ccccc4)nc(-c4ccccc4)n3)c2)cc1-c1cccc(-c2nc(-c3ccccc3)nc(-c3ccccc3)n2)c1 ; Cc1ccccc1-c1nc(-c2ccccc2)nc(-c2cccc(-c3cccc(-c4cccc(-c5nc(-c6ccccc6C)nc(-c6ccccc6C)n5)c4)c3)c2)n1]

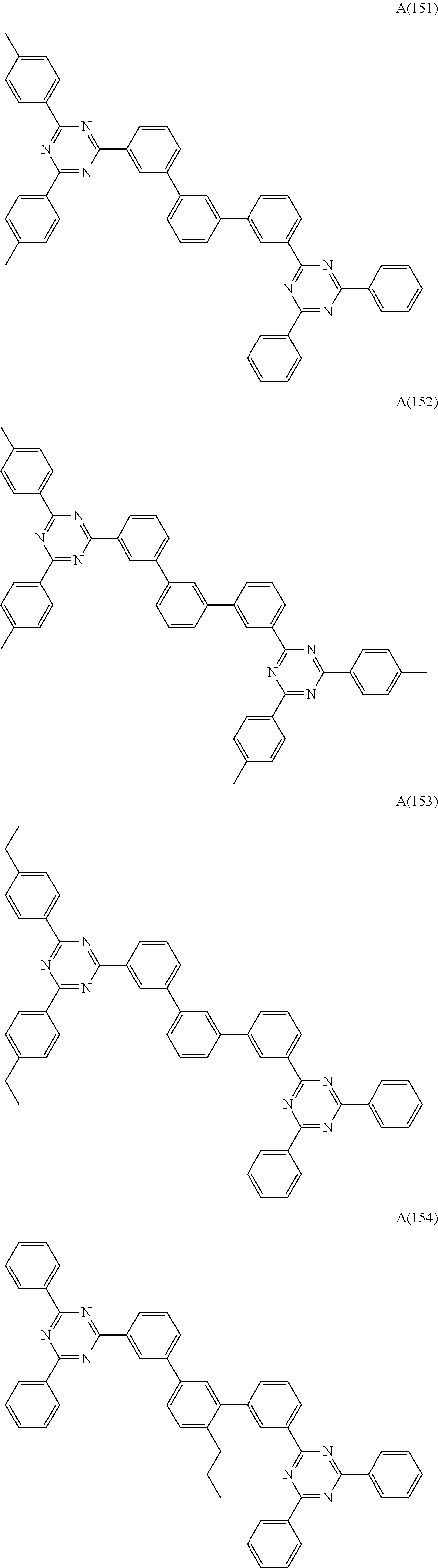 [SMILES: Cc1ccc(-c2nc(-c3ccc(C)cc3)nc(-c3cccc(-c4cccc(-c5cccc(-c6nc(-c7ccccc7)nc(-c7ccccc7)n6)c5)c4)c3)n2)cc1 ; Cc1ccc(-c2nc(-c3ccc(C)cc3)nc(-c3cccc(-c4cccc(-c5cccc(-c6nc(-c7ccc(C)cc7)nc(-c7ccc(C)cc7)n6)c5)c4)c3)n2)cc1 ; CCc1ccc(-c2nc(-c3ccc(CC)cc3)nc(-c3cccc(-c4cccc(-c5cccc(-c6nc(-c7ccccc7)nc(-c7ccccc7)n6)c5)c4)c3)n2)cc1 ; CCCc1ccc(-c2cccc(-c3nc(-c4ccccc4)nc(-c4ccccc4)n3)c2)cc1-c1cccc(-c2nc(-c3ccccc3)nc(-c3ccccc3)n2)c1]

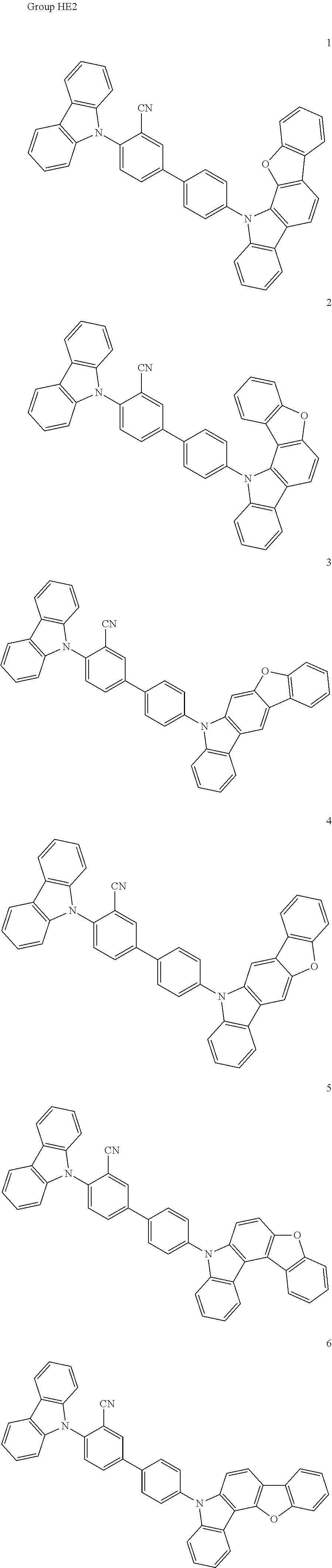 [SMILES: N#Cc1cc(-c2ccc(-n3c4ccccc4c4ccc5c6ccccc6oc5c43)cc2)ccc1-n1c2ccccc2c2ccccc21 ; N#Cc1cc(-c2ccc(-n3c4ccccc4c4ccc5oc6ccccc6c5c43)cc2)ccc1-n1c2ccccc2c2ccccc21 ; N#Cc1cc(-c2ccc(-n3c4ccccc4c4cc5c(cc43)oc3ccccc35)cc2)ccc1-n1c2ccccc2c2ccccc21 ; N#Cc1cc(-c2ccc(-n3c4ccccc4c4cc5oc6ccccc6c5cc43)cc2)ccc1-n1c2ccccc2c2ccccc21 ; N#Cc1cc(-c2ccc(-n3c4ccccc4c4c5c(ccc43)oc3ccccc35)cc2)ccc1-n1c2ccccc2c2ccccc21 ; N#Cc1cc(-c2ccc(-n3c4ccccc4c4c5oc6ccccc6c5ccc43)cc2)ccc1-n1c2ccccc2c2ccccc21]

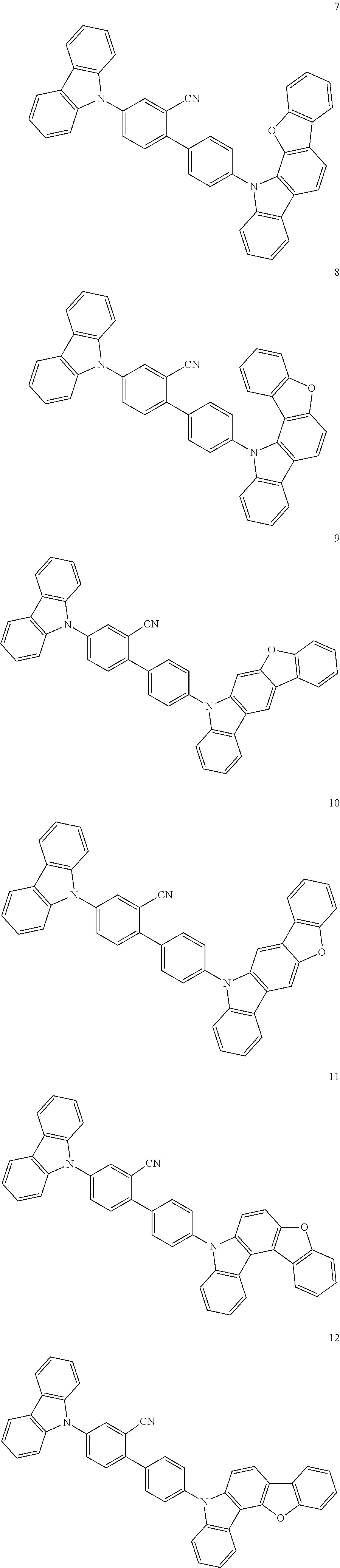 [SMILES: N#Cc1cc(-n2c3ccccc3c3ccccc32)ccc1-c1ccc(-n2c3ccccc3c3ccc4c5ccccc5oc4c32)cc1 ; N#Cc1cc(-n2c3ccccc3c3ccccc32)ccc1-c1ccc(-n2c3ccccc3c3ccc4oc5ccccc5c4c32)cc1 ; N#Cc1cc(-n2c3ccccc3c3ccccc32)ccc1-c1ccc(-n2c3ccccc3c3cc4c(cc32)oc2ccccc24)cc1 ; N#Cc1cc(-n2c3ccccc3c3ccccc32)ccc1-c1ccc(-n2c3ccccc3c3cc4oc5ccccc5c4cc32)cc1 ; N#Cc1cc(-n2c3ccccc3c3ccccc32)ccc1-c1ccc(-n2c3ccccc3c3c4c(ccc32)oc2ccccc24)cc1 ; N#Cc1cc(-n2c3ccccc3c3ccccc32)ccc1-c1ccc(-n2c3ccccc3c3c4oc5ccccc5c4ccc32)cc1]

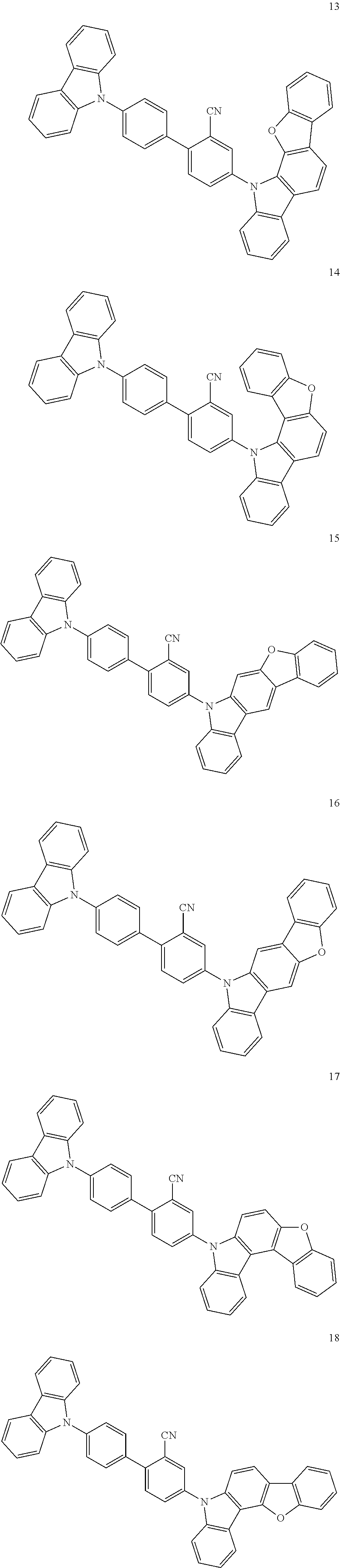 [SMILES: N#Cc1cc(-n2c3ccccc3c3ccc4c5ccccc5oc4c32)ccc1-c1ccc(-n2c3ccccc3c3ccccc32)cc1 ; N#Cc1cc(-n2c3ccccc3c3ccc4oc5ccccc5c4c32)ccc1-c1ccc(-n2c3ccccc3c3ccccc32)cc1 ; N#Cc1cc(-n2c3ccccc3c3cc4c(cc32)oc2ccccc24)ccc1-c1ccc(-n2c3ccccc3c3ccccc32)cc1 ; N#Cc1cc(-n2c3ccccc3c3cc4oc5ccccc5c4cc32)ccc1-c1ccc(-n2c3ccccc3c3ccccc32)cc1 ; N#Cc1cc(-n2c3ccccc3c3c4c(ccc32)oc2ccccc24)ccc1-c1ccc(-n2c3ccccc3c3ccccc32)cc1 ; N#Cc1cc(-n2c3ccccc3c3c4oc5ccccc5c4ccc32)ccc1-c1ccc(-n2c3ccccc3c3ccccc32)cc1]

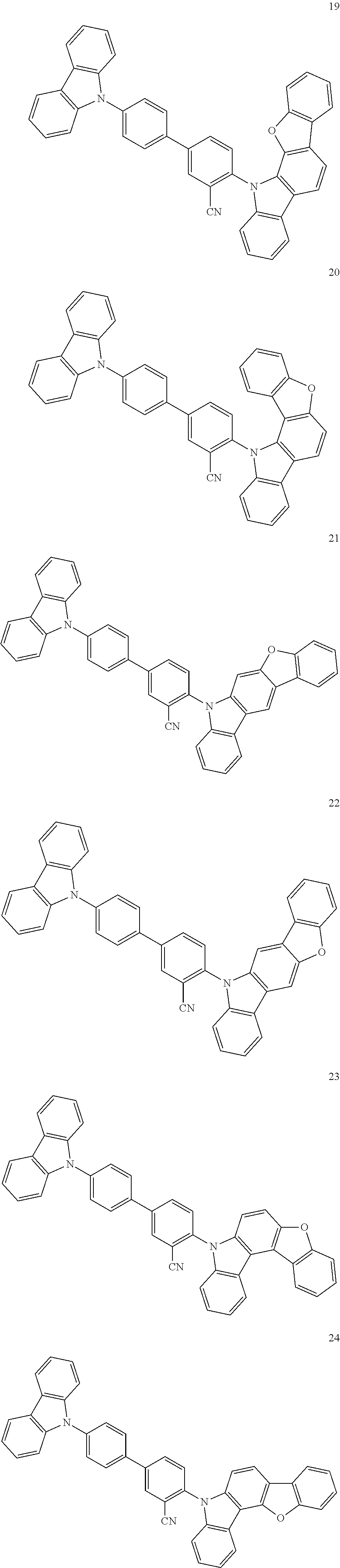 [SMILES: N#Cc1cc(-c2ccc(-n3c4ccccc4c4ccccc43)cc2)ccc1-n1c2ccccc2c2ccc3c4ccccc4oc3c21 ; N#Cc1cc(-c2ccc(-n3c4ccccc4c4ccccc43)cc2)ccc1-n1c2ccccc2c2ccc3oc4ccccc4c3c21 ; N#Cc1cc(-c2ccc(-n3c4ccccc4c4ccccc43)cc2)ccc1-n1c2ccccc2c2cc3c(cc21)oc1ccccc13 ; N#Cc1cc(-c2ccc(-n3c4ccccc4c4ccccc43)cc2)ccc1-n1c2ccccc2c2cc3oc4ccccc4c3cc21 ; N#Cc1cc(-c2ccc(-n3c4ccccc4c4ccccc43)cc2)ccc1-n1c2ccccc2c2c3c(ccc21)oc1ccccc13 ; N#Cc1cc(-c2ccc(-n3c4ccccc4c4ccccc43)cc2)ccc1-n1c2ccccc2c2c3oc4ccccc4c3ccc21]

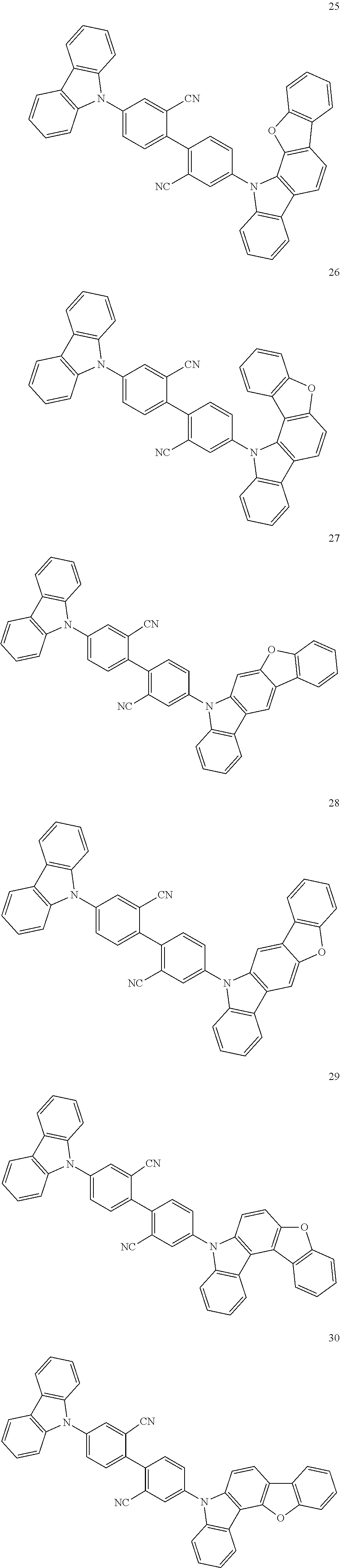 [SMILES: N#Cc1cc(-n2c3ccccc3c3ccccc32)ccc1-c1ccc(-n2c3ccccc3c3ccc4c5ccccc5oc4c32)cc1C#N ; N#Cc1cc(-n2c3ccccc3c3ccccc32)ccc1-c1ccc(-n2c3ccccc3c3ccc4oc5ccccc5c4c32)cc1C#N ; N#Cc1cc(-n2c3ccccc3c3ccccc32)ccc1-c1ccc(-n2c3ccccc3c3cc4c(cc32)oc2ccccc24)cc1C#N ; N#Cc1cc(-n2c3ccccc3c3ccccc32)ccc1-c1ccc(-n2c3ccccc3c3cc4oc5ccccc5c4cc32)cc1C#N ; N#Cc1cc(-n2c3ccccc3c3ccccc32)ccc1-c1ccc(-n2c3ccccc3c3c4c(ccc32)oc2ccccc24)cc1C#N ; N#Cc1cc(-n2c3ccccc3c3ccccc32)ccc1-c1ccc(-n2c3ccccc3c3c4oc5ccccc5c4ccc32)cc1C#N]

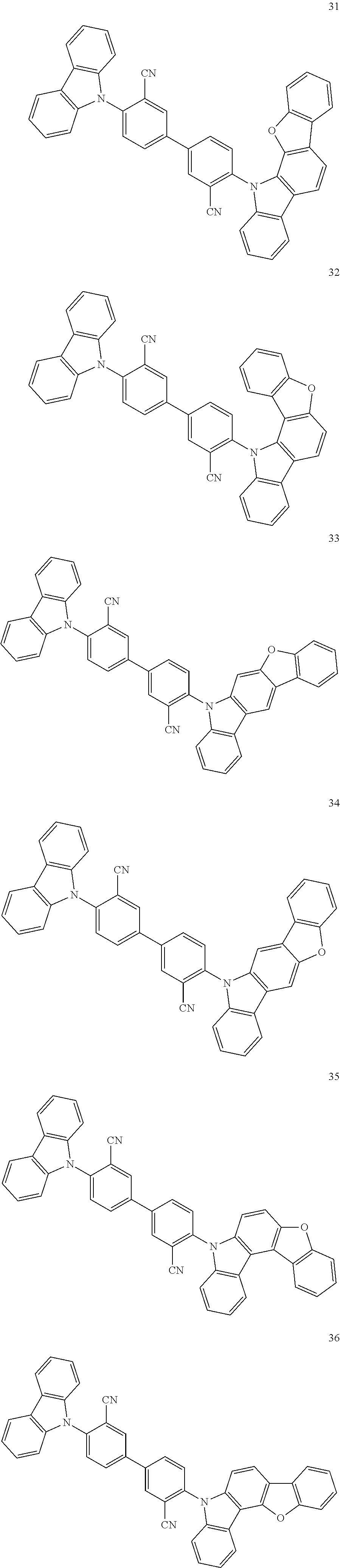 [SMILES: N#Cc1cc(-c2ccc(-n3c4ccccc4c4ccc5c6ccccc6oc5c43)c(C#N)c2)ccc1-n1c2ccccc2c2ccccc21 ; N#Cc1cc(-c2ccc(-n3c4ccccc4c4ccc5oc6ccccc6c5c43)c(C#N)c2)ccc1-n1c2ccccc2c2ccccc21 ; N#Cc1cc(-c2ccc(-n3c4ccccc4c4cc5c(cc43)oc3ccccc35)c(C#N)c2)ccc1-n1c2ccccc2c2ccccc21 ; N#Cc1cc(-c2ccc(-n3c4ccccc4c4cc5oc6ccccc6c5cc43)c(C#N)c2)ccc1-n1c2ccccc2c2ccccc21 ; N#Cc1cc(-c2ccc(-n3c4ccccc4c4c5c(ccc43)oc3ccccc35)c(C#N)c2)ccc1-n1c2ccccc2c2ccccc21 ; N#Cc1cc(-c2ccc(-n3c4ccccc4c4c5oc6ccccc6c5ccc43)c(C#N)c2)ccc1-n1c2ccccc2c2ccccc21]

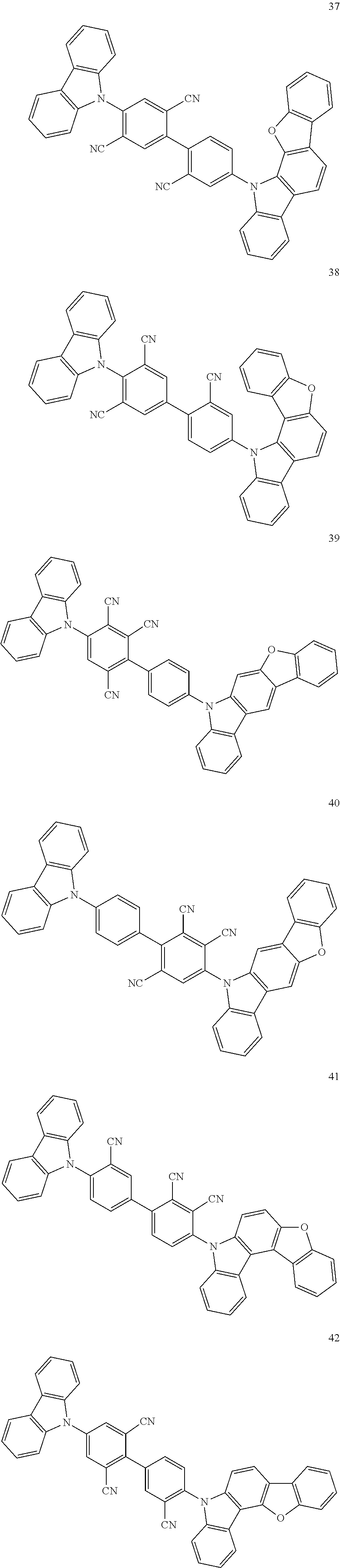

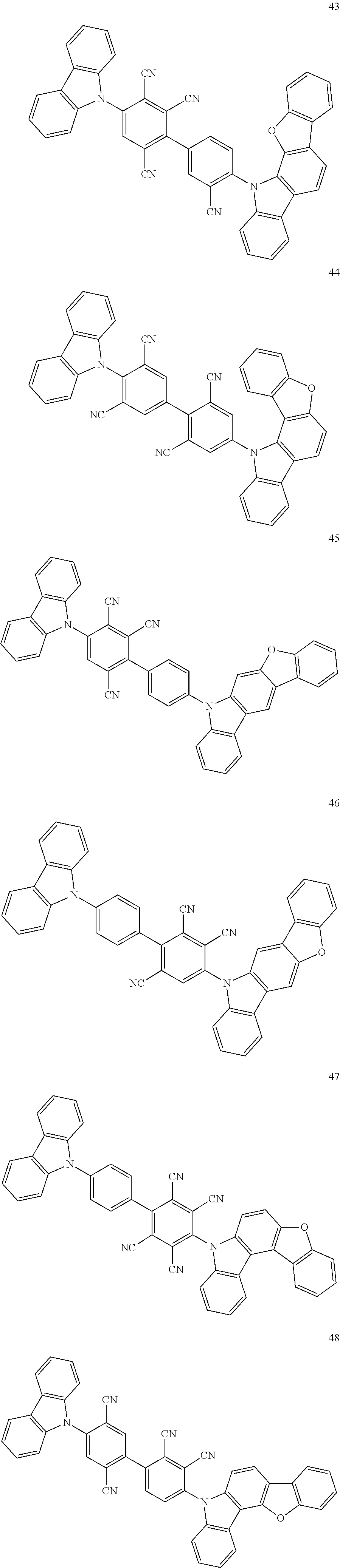

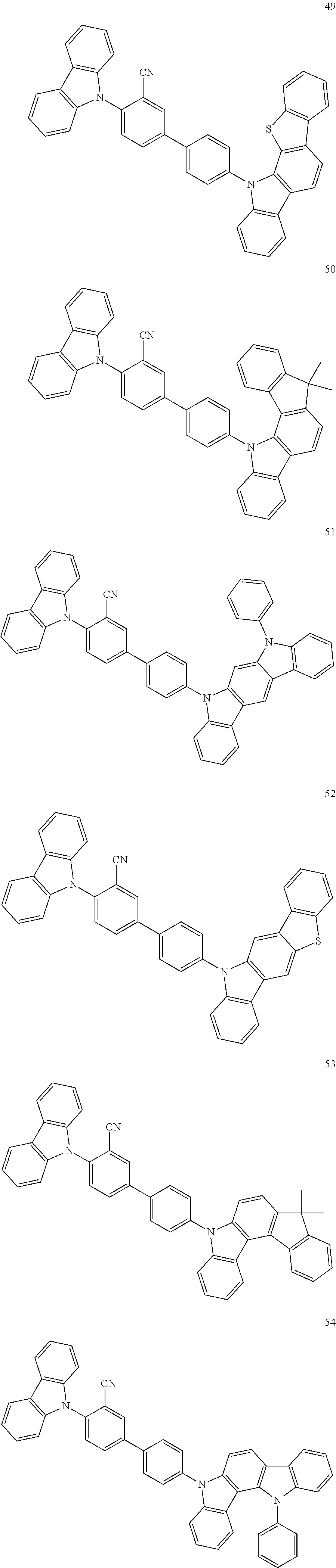

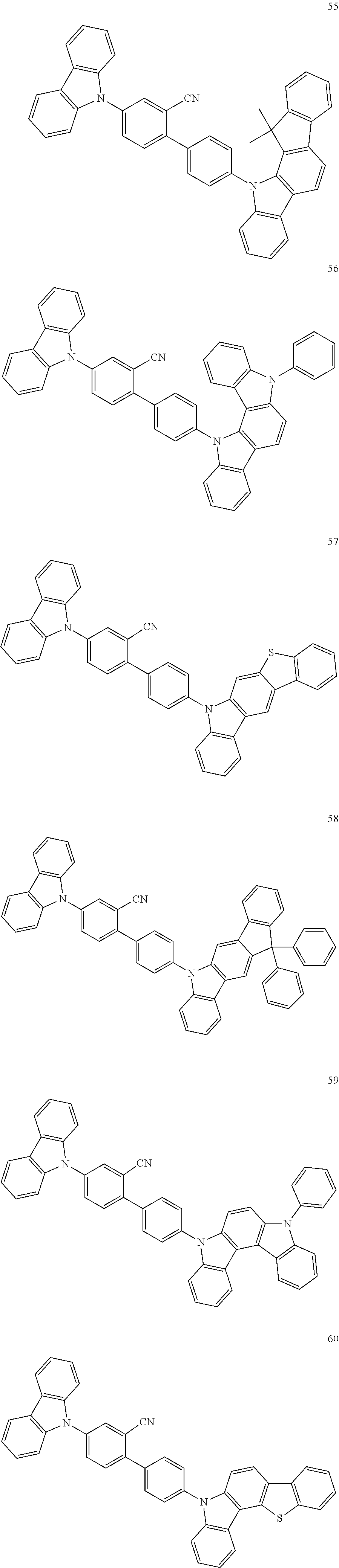

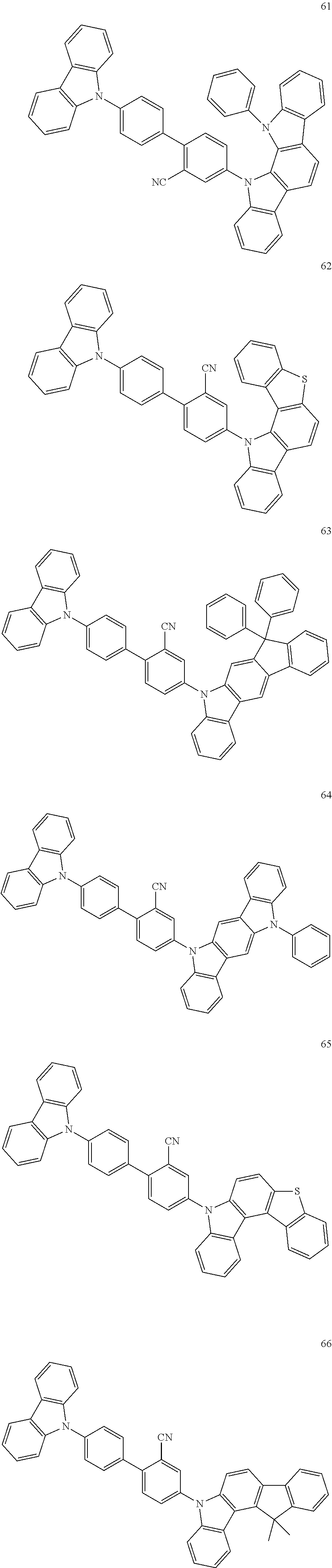

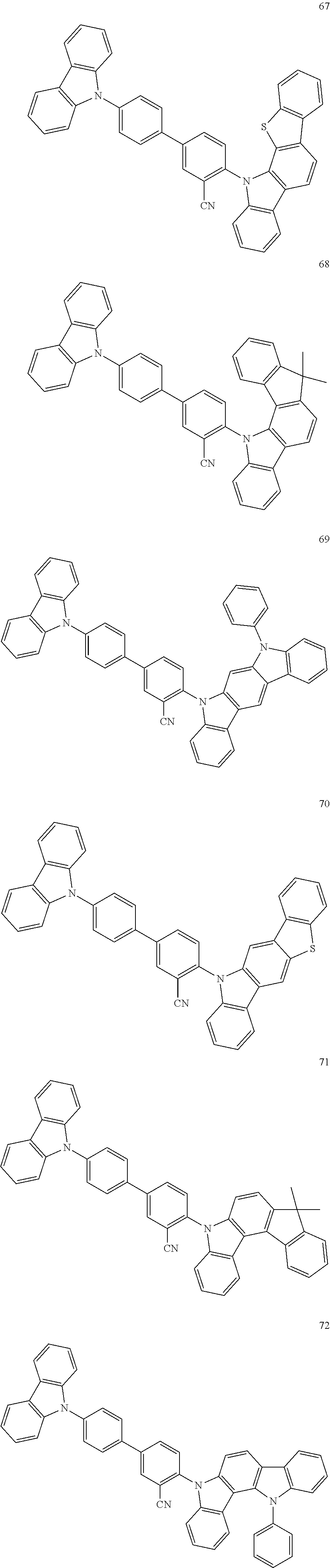 [SMILES: N#Cc1cc(-c2ccc(-n3c4ccccc4c4ccccc43)cc2)ccc1-n1c2ccccc2c2ccc3c4ccccc4sc3c21 ; CC1(C)c2ccccc2-c2c1ccc1c3ccccc3n(-c3ccc(-c4ccc(-n5c6ccccc6c6ccccc65)cc4)cc3C#N)c21 ; N#Cc1cc(-c2ccc(-n3c4ccccc4c4ccccc43)cc2)ccc1-n1c2ccccc2c2cc3c4ccccc4n(-c4ccccc4)c3cc21 ; N#Cc1cc(-c2ccc(-n3c4ccccc4c4ccccc43)cc2)ccc1-n1c2ccccc2c2cc3sc4ccccc4c3cc21 ; CC1(C)c2ccccc2-c2c1ccc1c2c2ccccc2n1-c1ccc(-c2ccc(-n3c4ccccc4c4ccccc43)cc2)cc1C#N ; N#Cc1cc(-c2ccc(-n3c4ccccc4c4ccccc43)cc2)ccc1-n1c2ccccc2c2c1ccc1c3ccccc3n(-c3ccccc3)c12]

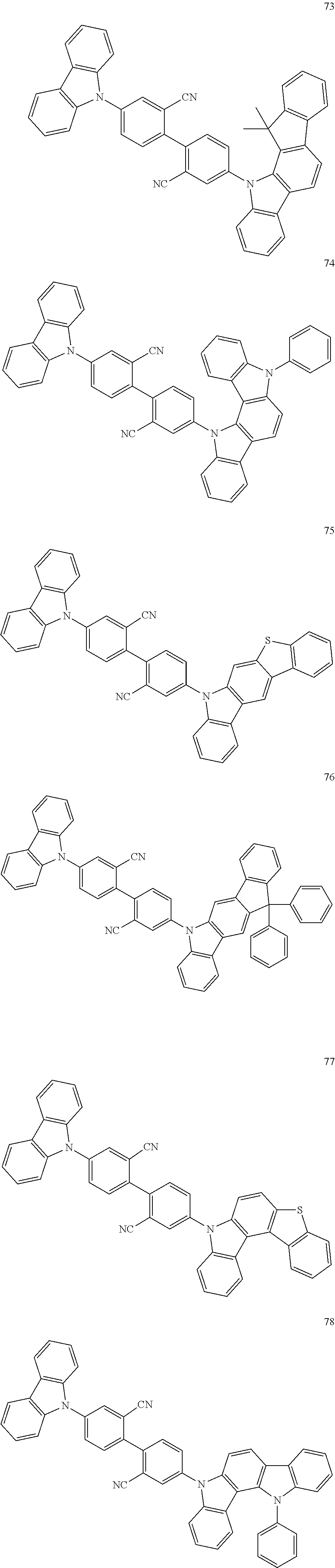

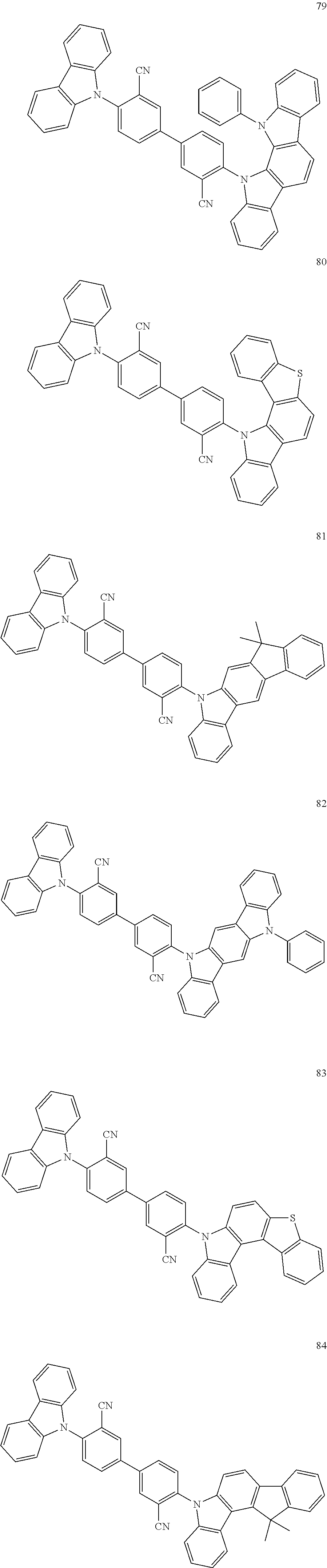

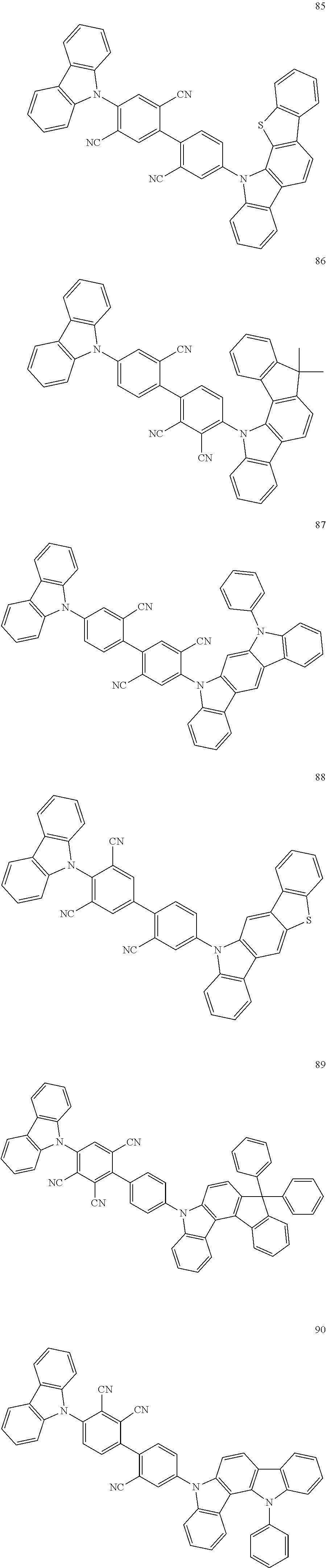

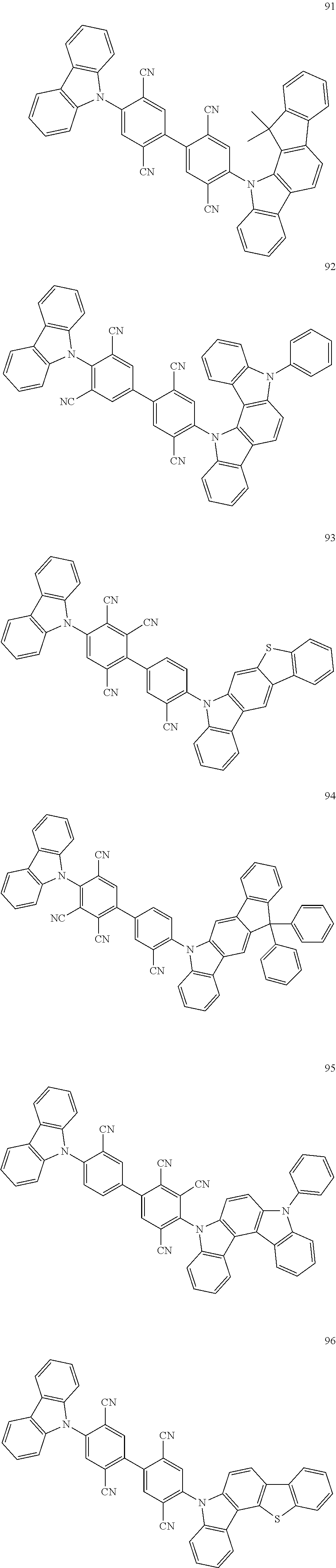

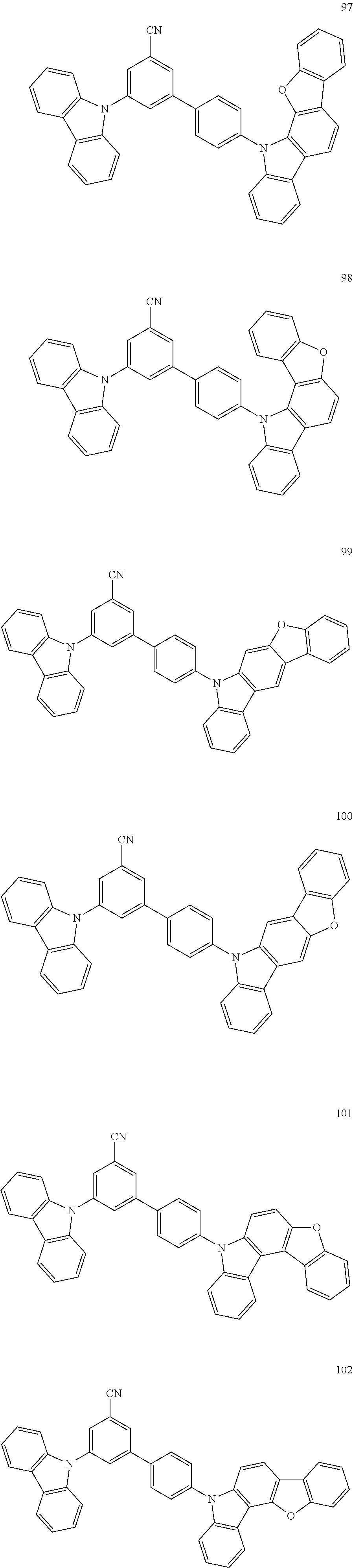 [SMILES: N#Cc1cc(-c2ccc(-n3c4ccccc4c4ccc5c6ccccc6oc5c43)cc2)cc(-n2c3ccccc3c3ccccc32)c1 ; N#Cc1cc(-c2ccc(-n3c4ccccc4c4ccc5oc6ccccc6c5c43)cc2)cc(-n2c3ccccc3c3ccccc32)c1 ; N#Cc1cc(-c2ccc(-n3c4ccccc4c4cc5c(cc43)oc3ccccc35)cc2)cc(-n2c3ccccc3c3ccccc32)c1 ; N#Cc1cc(-c2ccc(-n3c4ccccc4c4cc5oc6ccccc6c5cc43)cc2)cc(-n2c3ccccc3c3ccccc32)c1 ; N#Cc1cc(-c2ccc(-n3c4ccccc4c4c5c(ccc43)oc3ccccc35)cc2)cc(-n2c3ccccc3c3ccccc32)c1 ; N#Cc1cc(-c2ccc(-n3c4ccccc4c4c5oc6ccccc6c5ccc43)cc2)cc(-n2c3ccccc3c3ccccc32)c1]

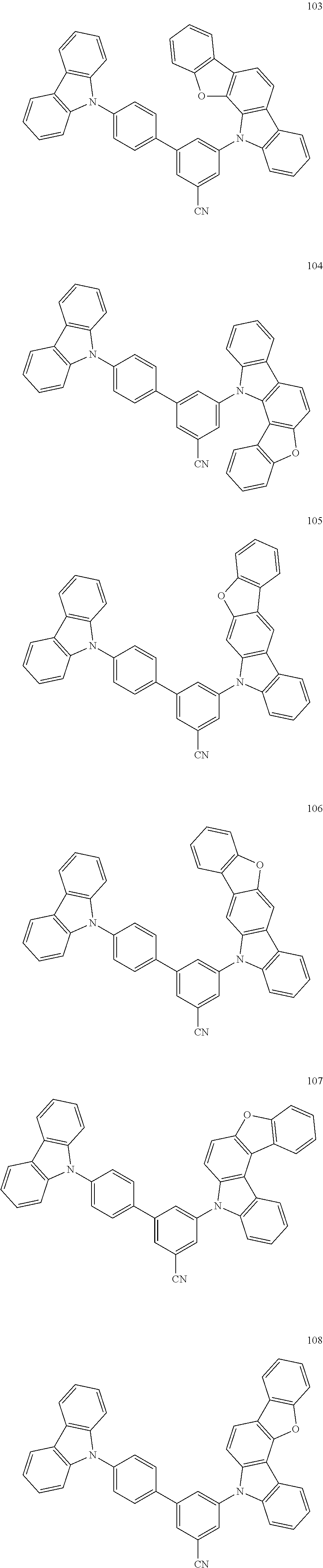 [SMILES: N#Cc1cc(-c2ccc(-n3c4ccccc4c4ccccc43)cc2)cc(-n2c3ccccc3c3ccc4c5ccccc5oc4c32)c1 ; N#Cc1cc(-c2ccc(-n3c4ccccc4c4ccccc43)cc2)cc(-n2c3ccccc3c3ccc4oc5ccccc5c4c32)c1 ; N#Cc1cc(-c2ccc(-n3c4ccccc4c4ccccc43)cc2)cc(-n2c3ccccc3c3cc4c(cc32)oc2ccccc24)c1 ; N#Cc1cc(-c2ccc(-n3c4ccccc4c4ccccc43)cc2)cc(-n2c3ccccc3c3cc4oc5ccccc5c4cc32)c1 ; N#Cc1cc(-c2ccc(-n3c4ccccc4c4ccccc43)cc2)cc(-n2c3ccccc3c3c4c(ccc32)oc2ccccc24)c1 ; N#Cc1cc(-c2ccc(-n3c4ccccc4c4ccccc43)cc2)cc(-n2c3ccccc3c3c4oc5ccccc5c4ccc32)c1]

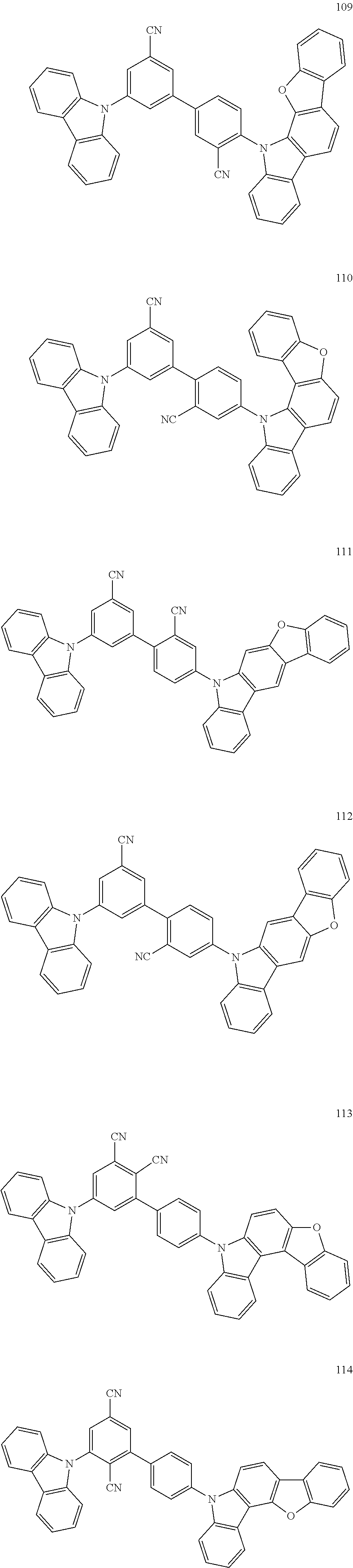

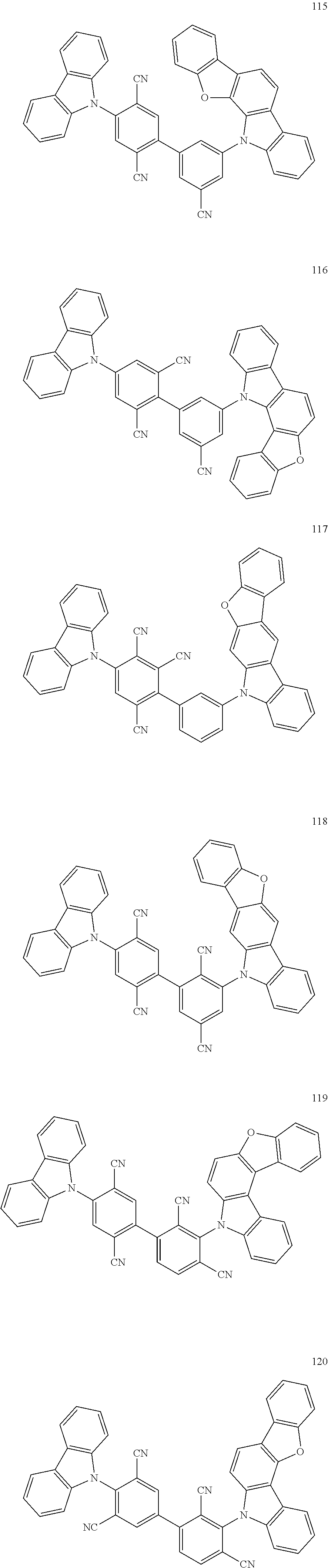

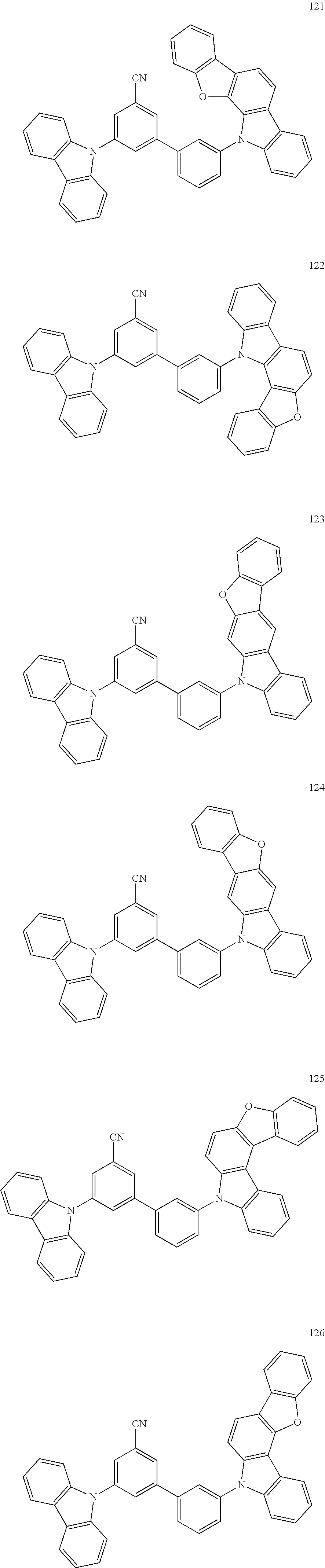 [SMILES: N#Cc1cc(-c2cccc(-n3c4ccccc4c4ccc5c6ccccc6oc5c43)c2)cc(-n2c3ccccc3c3ccccc32)c1 ; N#Cc1cc(-c2cccc(-n3c4ccccc4c4ccc5oc6ccccc6c5c43)c2)cc(-n2c3ccccc3c3ccccc32)c1 ; N#Cc1cc(-c2cccc(-n3c4ccccc4c4cc5c(cc43)oc3ccccc35)c2)cc(-n2c3ccccc3c3ccccc32)c1 ; N#Cc1cc(-c2cccc(-n3c4ccccc4c4cc5oc6ccccc6c5cc43)c2)cc(-n2c3ccccc3c3ccccc32)c1 ; N#Cc1cc(-c2cccc(-n3c4ccccc4c4c5c(ccc43)oc3ccccc35)c2)cc(-n2c3ccccc3c3ccccc32)c1 ; N#Cc1cc(-c2cccc(-n3c4ccccc4c4c5oc6ccccc6c5ccc43)c2)cc(-n2c3ccccc3c3ccccc32)c1]

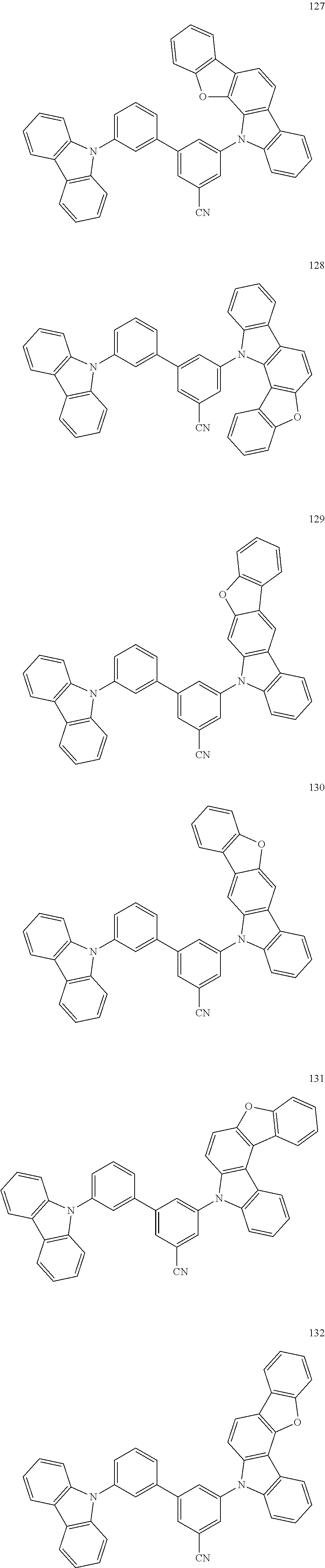 [SMILES: N#Cc1cc(-c2cccc(-n3c4ccccc4c4ccccc43)c2)cc(-n2c3ccccc3c3ccc4c5ccccc5oc4c32)c1 ; N#Cc1cc(-c2cccc(-n3c4ccccc4c4ccccc43)c2)cc(-n2c3ccccc3c3ccc4oc5ccccc5c4c32)c1 ; N#Cc1cc(-c2cccc(-n3c4ccccc4c4ccccc43)c2)cc(-n2c3ccccc3c3cc4c(cc32)oc2ccccc24)c1 ; N#Cc1cc(-c2cccc(-n3c4ccccc4c4ccccc43)c2)cc(-n2c3ccccc3c3cc4oc5ccccc5c4cc32)c1 ; N#Cc1cc(-c2cccc(-n3c4ccccc4c4ccccc43)c2)cc(-n2c3ccccc3c3c4c(ccc32)oc2ccccc24)c1 ; N#Cc1cc(-c2cccc(-n3c4ccccc4c4ccccc43)c2)cc(-n2c3ccccc3c3c4oc5ccccc5c4ccc32)c1]

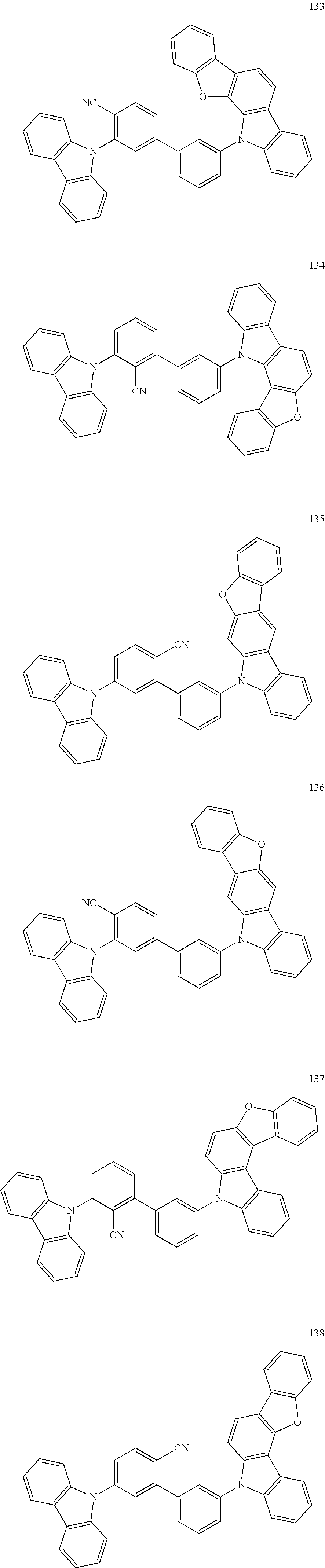 [SMILES: N#Cc1ccc(-c2cccc(-n3c4ccccc4c4ccc5c6ccccc6oc5c43)c2)cc1-n1c2ccccc2c2ccccc21 ; N#Cc1c(-c2cccc(-n3c4ccccc4c4ccc5oc6ccccc6c5c43)c2)cccc1-n1c2ccccc2c2ccccc21 ; N#Cc1ccc(-n2c3ccccc3c3ccccc32)cc1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)cc1-n1c2ccccc2c2ccccc21 ; N#Cc1c(-c2cccc(-n3c4ccccc4c4c5c(ccc43)oc3ccccc35)c2)cccc1-n1c2ccccc2c2ccccc21 ; N#Cc1ccc(-n2c3ccccc3c3ccccc32)cc1-c1cccc(-n2c3ccccc3c3c4oc5ccccc5c4ccc32)c1]

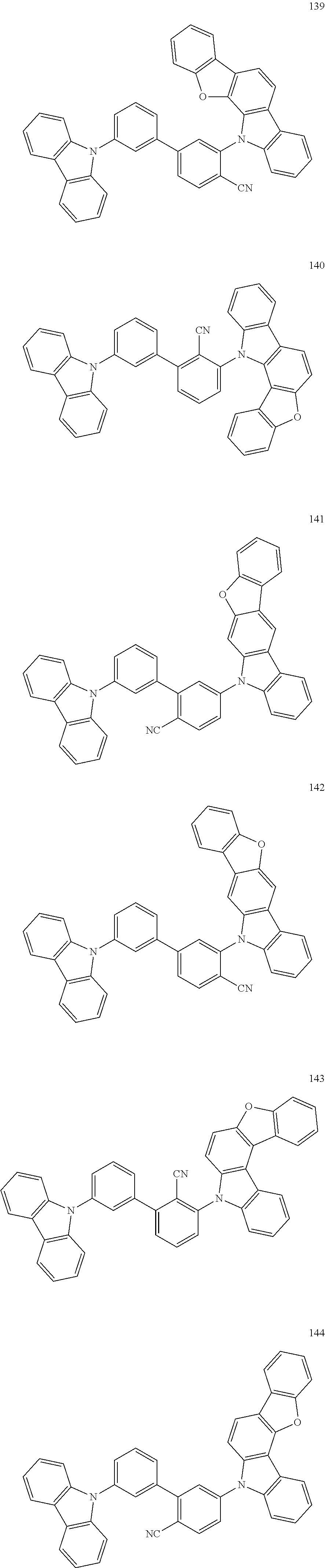 [SMILES: N#Cc1ccc(-c2cccc(-n3c4ccccc4c4ccccc43)c2)cc1-n1c2ccccc2c2ccc3c4ccccc4oc3c21 ; N#Cc1c(-c2cccc(-n3c4ccccc4c4ccccc43)c2)cccc1-n1c2ccccc2c2ccc3oc4ccccc4c3c21 ; N#Cc1ccc(-n2c3ccccc3c3cc4c(cc32)oc2ccccc24)cc1-c1cccc(-n2c3ccccc3c3ccccc32)c1 ; N#Cc1ccc(-c2cccc(-n3c4ccccc4c4ccccc43)c2)cc1-n1c2ccccc2c2cc3oc4ccccc4c3cc21 ; N#Cc1c(-c2cccc(-n3c4ccccc4c4ccccc43)c2)cccc1-n1c2ccccc2c2c3c(ccc21)oc1ccccc13 ; N#Cc1ccc(-n2c3ccccc3c3c4oc5ccccc5c4ccc32)cc1-c1cccc(-n2c3ccccc3c3ccccc32)c1]

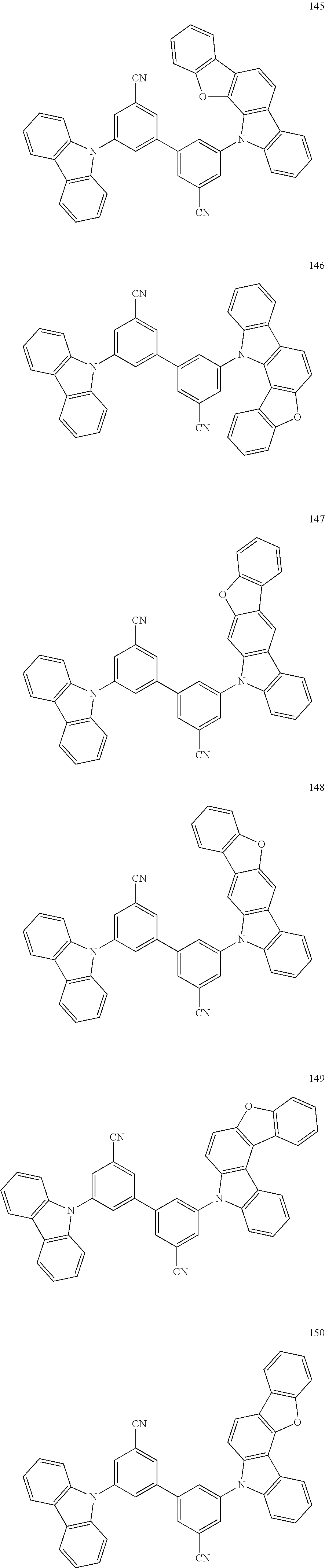

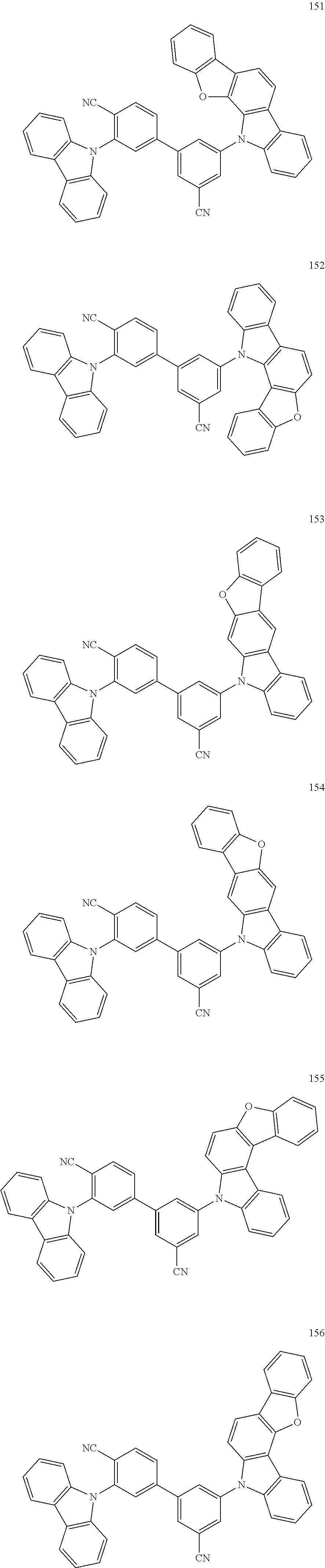

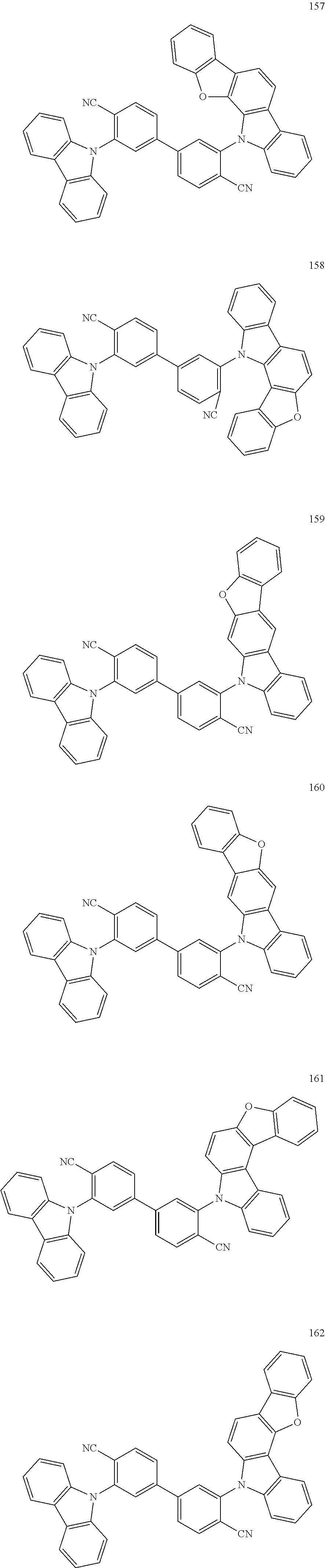 [SMILES: N#Cc1ccc(-c2ccc(C#N)c(-n3c4ccccc4c4ccc5c6ccccc6oc5c43)c2)cc1-n1c2ccccc2c2ccccc21 ; N#Cc1ccc(-c2ccc(C#N)c(-n3c4ccccc4c4ccc5oc6ccccc6c5c43)c2)cc1-n1c2ccccc2c2ccccc21 ; N#Cc1ccc(-c2ccc(C#N)c(-n3c4ccccc4c4cc5c(cc43)oc3ccccc35)c2)cc1-n1c2ccccc2c2ccccc21 ; N#Cc1ccc(-c2ccc(C#N)c(-n3c4ccccc4c4cc5oc6ccccc6c5cc43)c2)cc1-n1c2ccccc2c2ccccc21 ; N#Cc1ccc(-c2ccc(C#N)c(-n3c4ccccc4c4c5c(ccc43)oc3ccccc35)c2)cc1-n1c2ccccc2c2ccccc21 ; N#Cc1ccc(-c2ccc(C#N)c(-n3c4ccccc4c4c5oc6ccccc6c5ccc43)c2)cc1-n1c2ccccc2c2ccccc21]

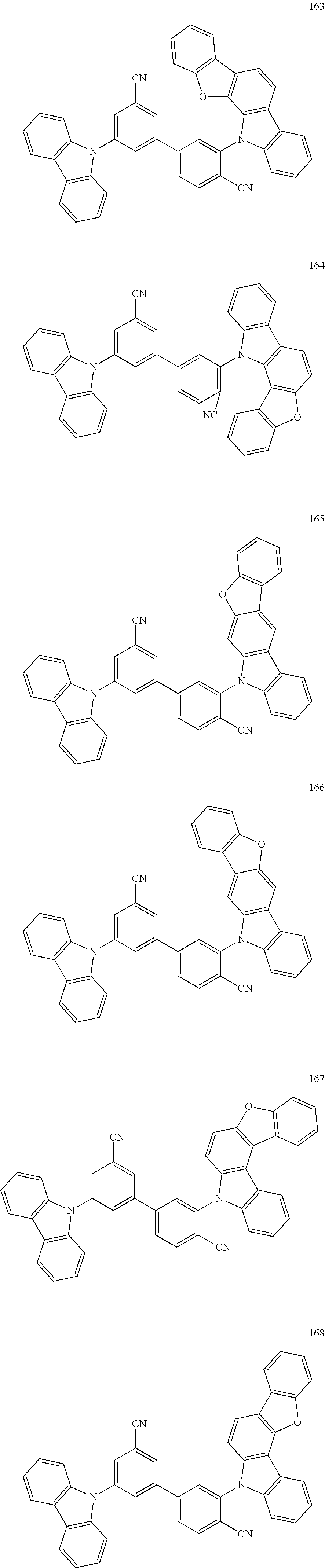

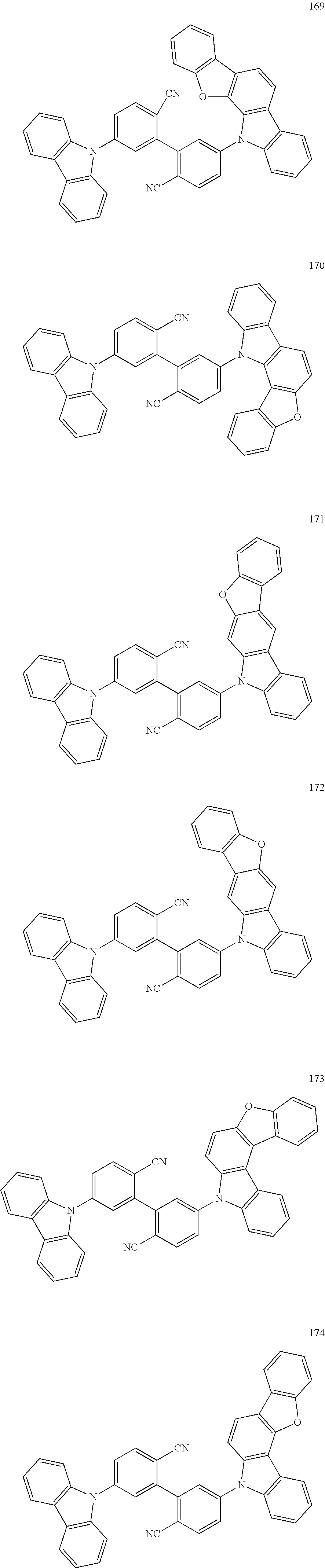 [SMILES: N#Cc1ccc(-n2c3ccccc3c3ccccc32)cc1-c1cc(-n2c3ccccc3c3ccc4c5ccccc5oc4c32)ccc1C#N ; N#Cc1ccc(-n2c3ccccc3c3ccccc32)cc1-c1cc(-n2c3ccccc3c3ccc4oc5ccccc5c4c32)ccc1C#N ; N#Cc1ccc(-n2c3ccccc3c3ccccc32)cc1-c1cc(-n2c3ccccc3c3cc4c(cc32)oc2ccccc24)ccc1C#N ; N#Cc1ccc(-n2c3ccccc3c3ccccc32)cc1-c1cc(-n2c3ccccc3c3cc4oc5ccccc5c4cc32)ccc1C#N ; N#Cc1ccc(-n2c3ccccc3c3ccccc32)cc1-c1cc(-n2c3ccccc3c3c4c(ccc32)oc2ccccc24)ccc1C#N ; N#Cc1ccc(-n2c3ccccc3c3ccccc32)cc1-c1cc(-n2c3ccccc3c3c4oc5ccccc5c4ccc32)ccc1C#N]

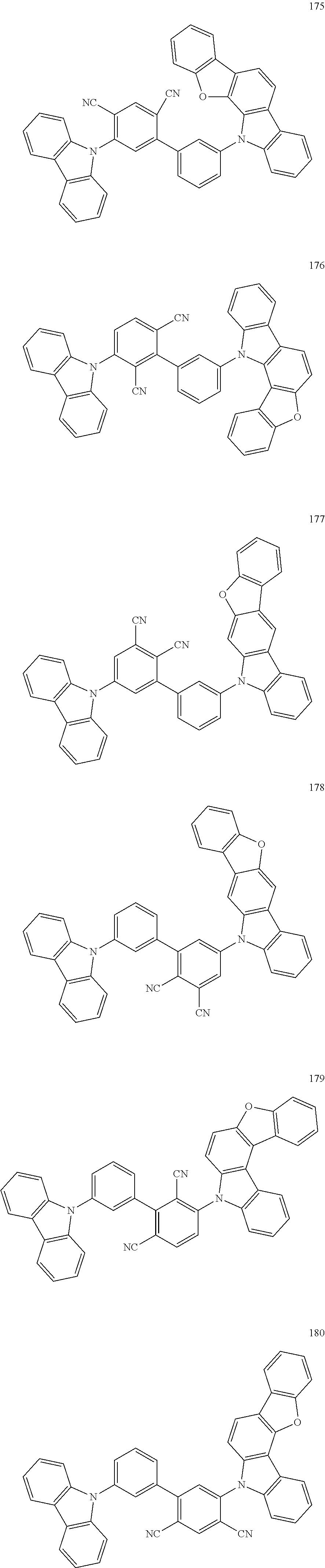 [SMILES: N#Cc1cc(C#N)c(-n2c3ccccc3c3ccccc32)cc1-c1cccc(-n2c3ccccc3c3ccc4c5ccccc5oc4c32)c1 ; N#Cc1ccc(-n2c3ccccc3c3ccccc32)c(C#N)c1-c1cccc(-n2c3ccccc3c3ccc4oc5ccccc5c4c32)c1 ; N#Cc1cc(-n2c3ccccc3c3ccccc32)cc(-c2cccc(-n3c4ccccc4c4cc5c(cc43)oc3ccccc35)c2)c1C#N ; N#Cc1cc(-n2c3ccccc3c3cc4oc5ccccc5c4cc32)cc(-c2cccc(-n3c4ccccc4c4ccccc43)c2)c1C#N ; N#Cc1ccc(-n2c3ccccc3c3c4c(ccc32)oc2ccccc24)c(C#N)c1-c1cccc(-n2c3ccccc3c3ccccc32)c1 ; N#Cc1cc(C#N)c(-n2c3ccccc3c3c4oc5ccccc5c4ccc32)cc1-c1cccc(-n2c3ccccc3c3ccccc32)c1]

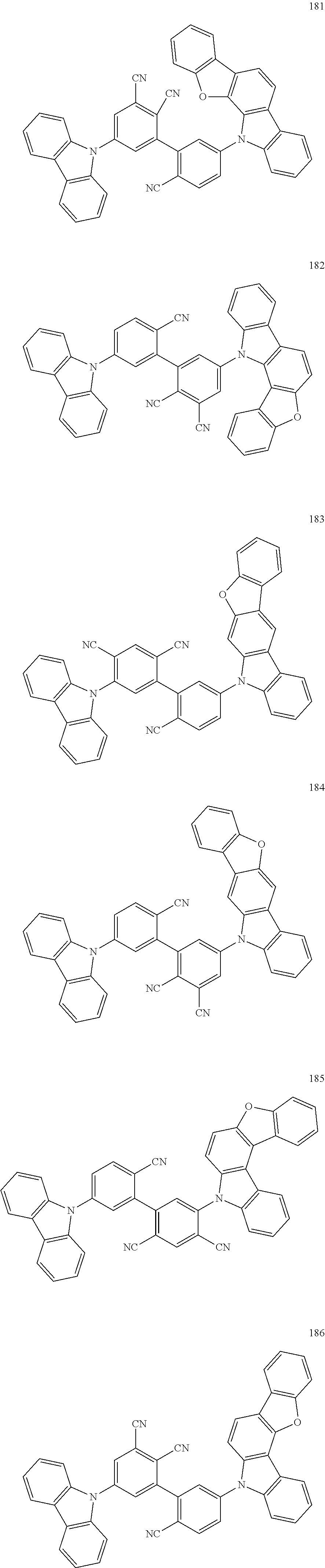

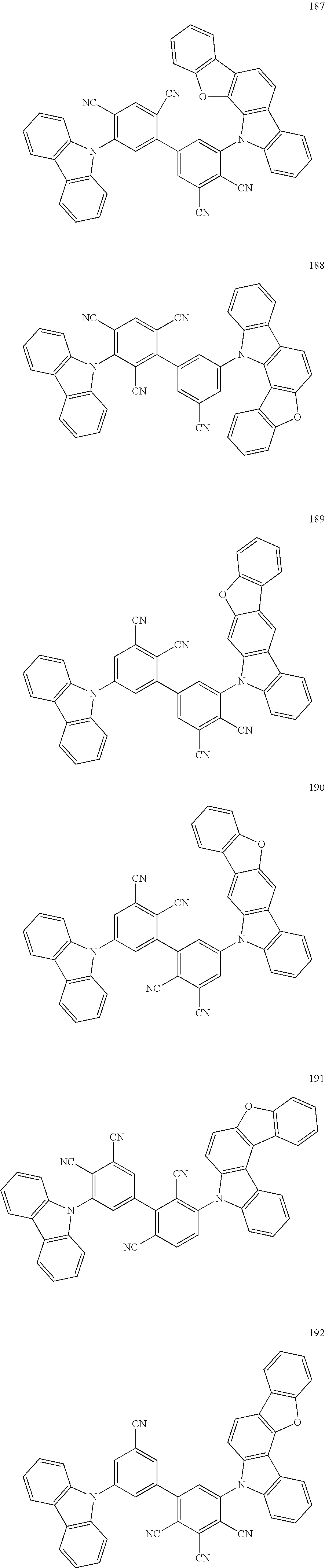

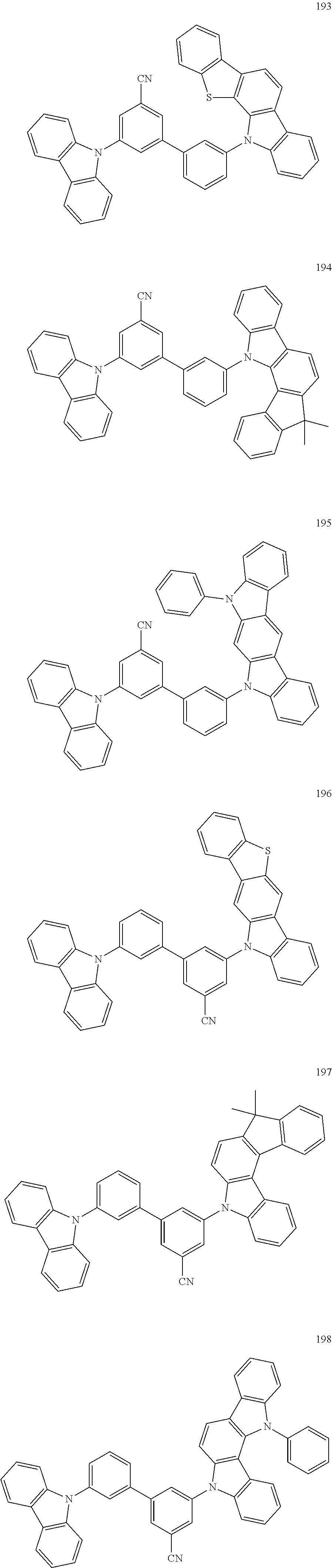

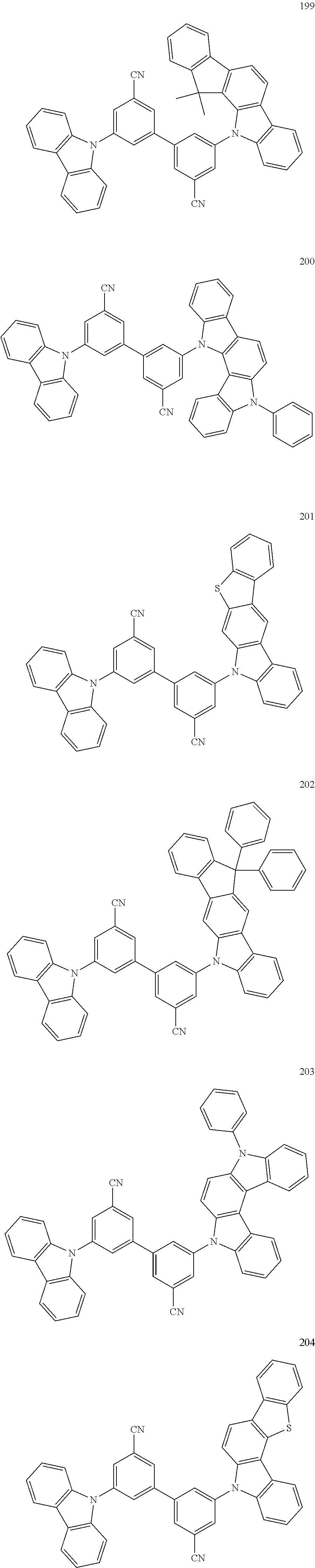

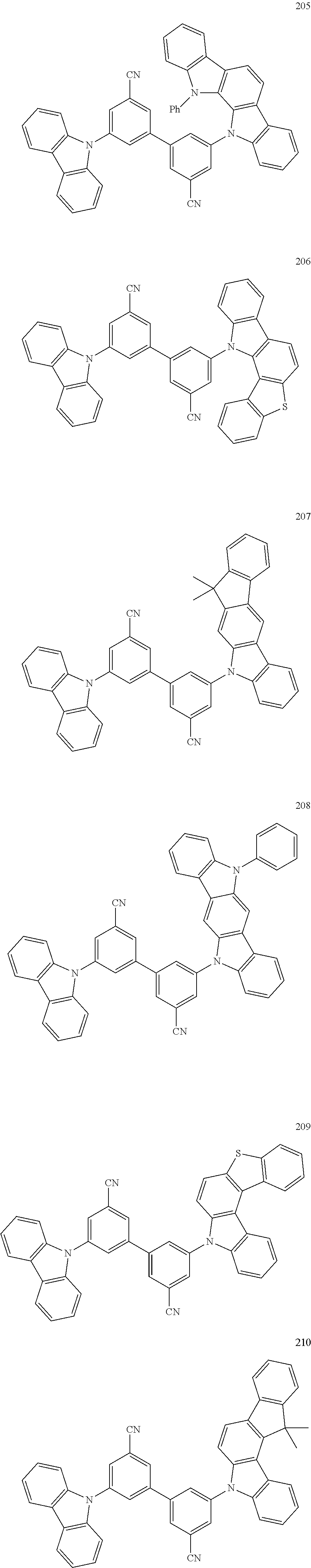

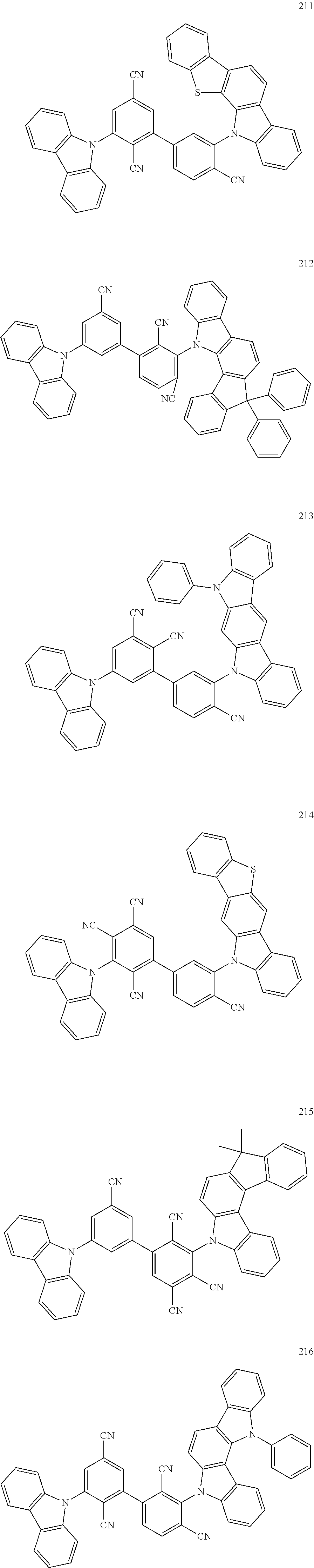

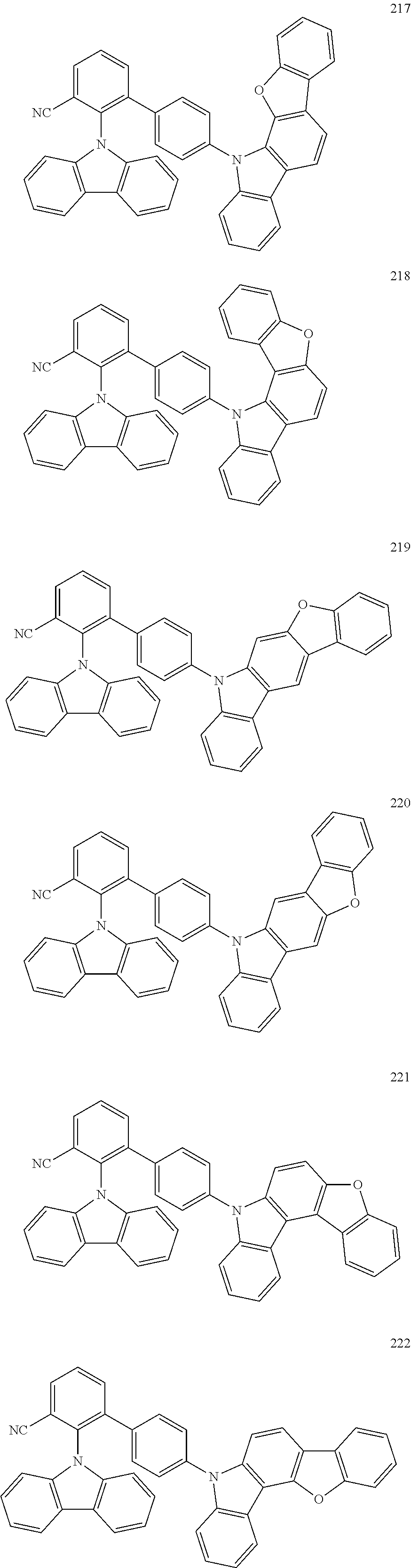

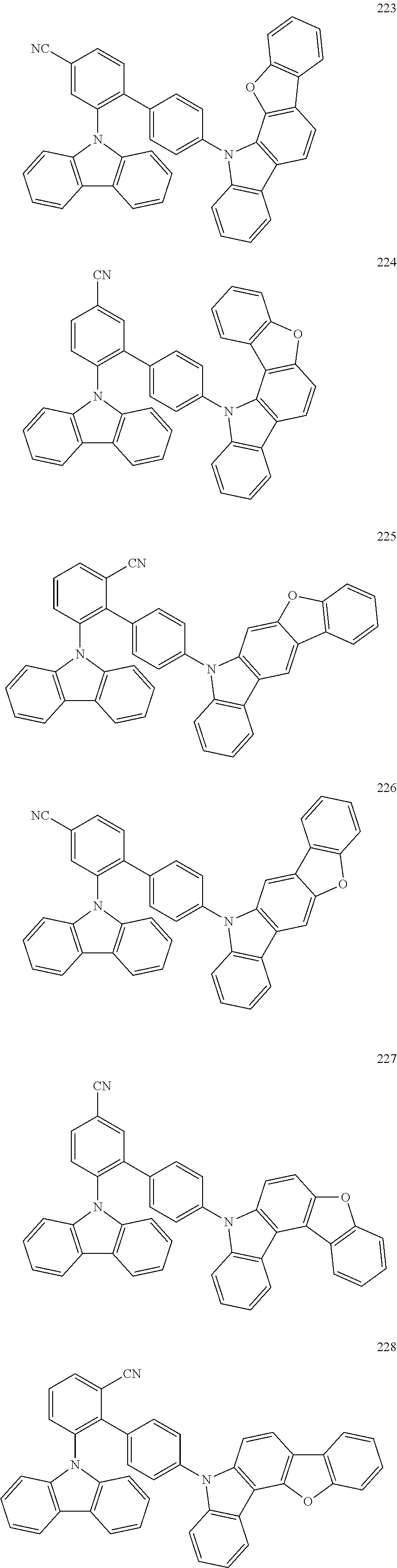 [SMILES: N#Cc1ccc(-c2ccc(-n3c4ccccc4c4ccc5c6ccccc6oc5c43)cc2)c(-n2c3ccccc3c3ccccc32)c1 ; N#Cc1ccc(-n2c3ccccc3c3ccccc32)c(-c2ccc(-n3c4ccccc4c4ccc5oc6ccccc6c5c43)cc2)c1 ; N#Cc1cccc(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)cc2)c1 ; N#Cc1cccc(-n2c3ccccc3c3ccccc32)c1-c1ccc(-n2c3ccccc3c3c4oc5ccccc5c4ccc32)cc1]

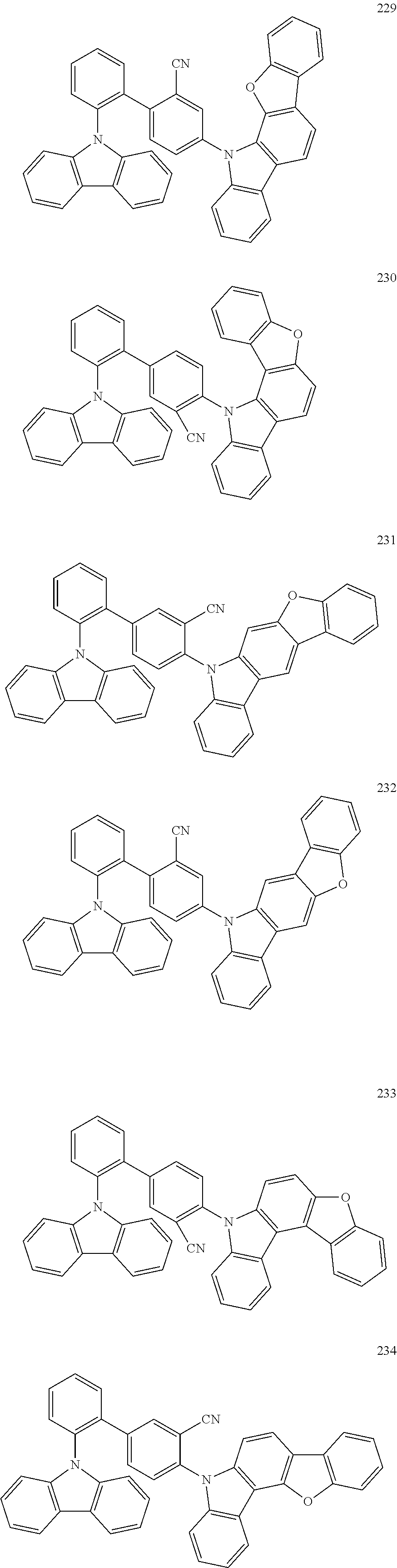 [SMILES: N#Cc1cc(-n2c3ccccc3c3ccc4c5ccccc5oc4c32)ccc1-c1ccccc1-n1c2ccccc2c2ccccc21 ; N#Cc1cc(-c2ccccc2-n2c3ccccc3c3ccccc32)ccc1-n1c2ccccc2c2ccc3oc4ccccc4c3c21 ; N#Cc1cc(-c2ccccc2-n2c3ccccc3c3ccccc32)ccc1-n1c2ccccc2c2cc3c(cc21)oc1ccccc13 ; N#Cc1cc(-n2c3ccccc3c3cc4oc5ccccc5c4cc32)ccc1-c1ccccc1-n1c2ccccc2c2ccccc21 ; N#Cc1cc(-c2ccccc2-n2c3ccccc3c3ccccc32)ccc1-n1c2ccccc2c2c3c(ccc21)oc1ccccc13 ; N#Cc1cc(-c2ccccc2-n2c3ccccc3c3ccccc32)ccc1-n1c2ccccc2c2c3oc4ccccc4c3ccc21]

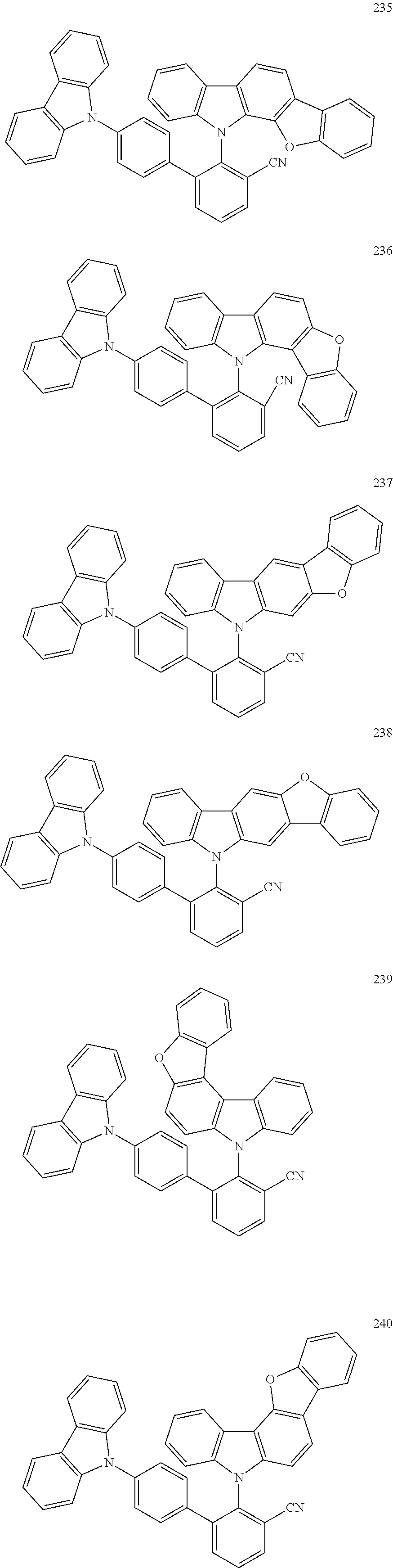 [SMILES: N#Cc1cccc(-c2ccc(-n3c4ccccc4c4ccccc43)cc2)c1-n1c2ccccc2c2ccc3c4ccccc4oc3c21 ; N#Cc1cccc(-c2ccc(-n3c4ccccc4c4ccccc43)cc2)c1-n1c2ccccc2c2ccc3oc4ccccc4c3c21 ; N#Cc1cccc(-c2ccc(-n3c4ccccc4c4ccccc43)cc2)c1-n1c2ccccc2c2cc3c(cc21)oc1ccccc13 ; N#Cc1cccc(-c2ccc(-n3c4ccccc4c4ccccc43)cc2)c1-n1c2ccccc2c2cc3oc4ccccc4c3cc21 ; N#Cc1cccc(-c2ccc(-n3c4ccccc4c4ccccc43)cc2)c1-n1c2ccccc2c2c3c(ccc21)oc1ccccc13 ; N#Cc1cccc(-c2ccc(-n3c4ccccc4c4ccccc43)cc2)c1-n1c2ccccc2c2c3oc4ccccc4c3ccc21]

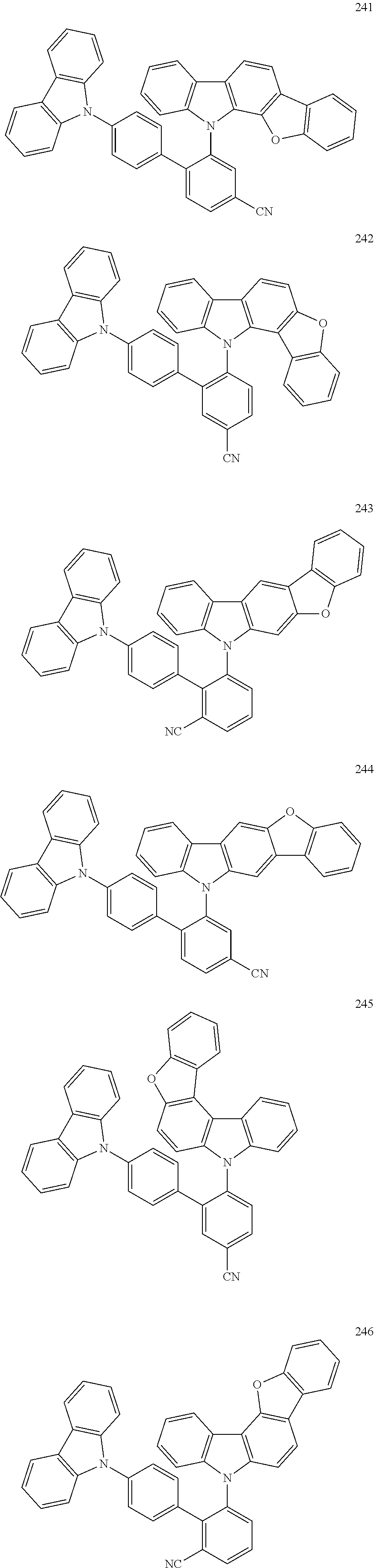 [SMILES: N#Cc1ccc(-c2ccc(-n3c4ccccc4c4ccccc43)cc2)c(-n2c3ccccc3c3ccc4c5ccccc5oc4c32)c1 ; N#Cc1ccc(-n2c3ccccc3c3ccc4oc5ccccc5c4c32)c(-c2ccc(-n3c4ccccc4c4ccccc43)cc2)c1 ; N#Cc1cccc(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1 ; N#Cc1ccc(-n2c3ccccc3c3c4c(ccc32)oc2ccccc24)c(-c2ccc(-n3c4ccccc4c4ccccc43)cc2)c1 ; N#Cc1cccc(-n2c3ccccc3c3c4oc5ccccc5c4ccc32)c1-c1ccc(-n2c3ccccc3c3ccccc32)cc1]

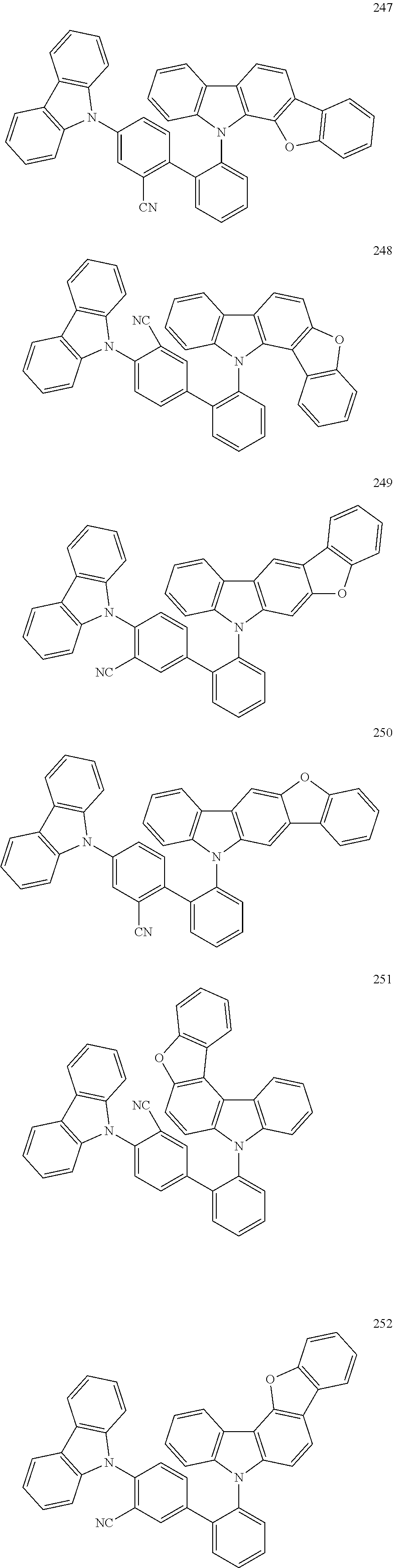 [SMILES: N#Cc1cc(-n2c3ccccc3c3ccccc32)ccc1-c1ccccc1-n1c2ccccc2c2ccc3c4ccccc4oc3c21 ; N#Cc1cc(-c2ccccc2-n2c3ccccc3c3ccc4oc5ccccc5c4c32)ccc1-n1c2ccccc2c2ccccc21 ; N#Cc1cc(-c2ccccc2-n2c3ccccc3c3cc4c(cc32)oc2ccccc24)ccc1-n1c2ccccc2c2ccccc21 ; N#Cc1cc(-n2c3ccccc3c3ccccc32)ccc1-c1ccccc1-n1c2ccccc2c2cc3oc4ccccc4c3cc21 ; N#Cc1cc(-c2ccccc2-n2c3ccccc3c3c4c(ccc32)oc2ccccc24)ccc1-n1c2ccccc2c2ccccc21 ; N#Cc1cc(-c2ccccc2-n2c3ccccc3c3c4oc5ccccc5c4ccc32)ccc1-n1c2ccccc2c2ccccc21]

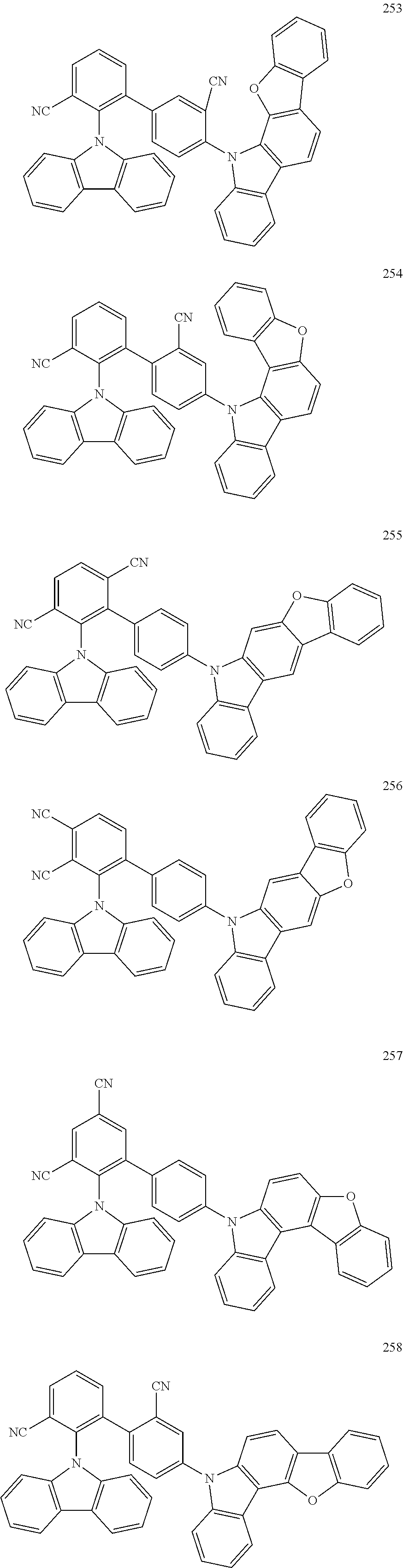

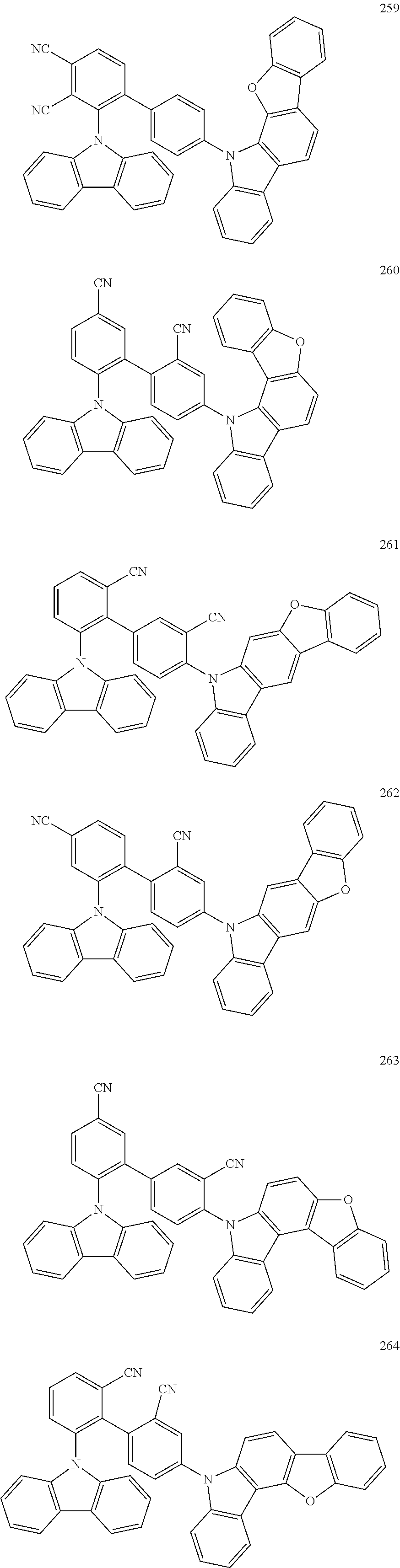 [SMILES: N#Cc1ccc(-c2ccc(-n3c4ccccc4c4ccc5c6ccccc6oc5c43)cc2)c(-n2c3ccccc3c3ccccc32)c1C#N ; N#Cc1ccc(-n2c3ccccc3c3ccccc32)c(-c2ccc(-n3c4ccccc4c4ccc5oc6ccccc6c5c43)cc2C#N)c1 ; N#Cc1cc(-c2c(C#N)cccc2-n2c3ccccc3c3ccccc32)ccc1-n1c2ccccc2c2cc3c(cc21)oc1ccccc13 ; N#Cc1ccc(-c2ccc(-n3c4ccccc4c4cc5oc6ccccc6c5cc43)cc2C#N)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)c(C#N)c2)c1 ; N#Cc1cc(-n2c3ccccc3c3c4oc5ccccc5c4ccc32)ccc1-c1c(C#N)cccc1-n1c2ccccc2c2ccccc21]

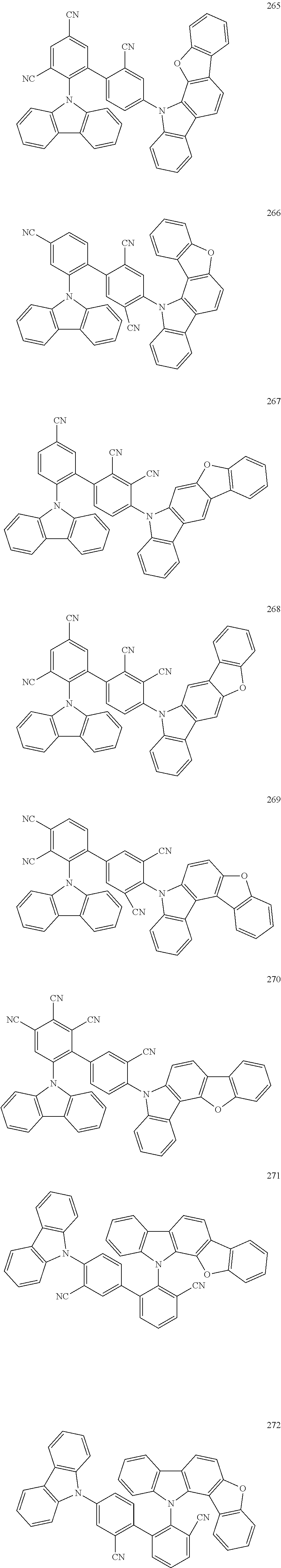

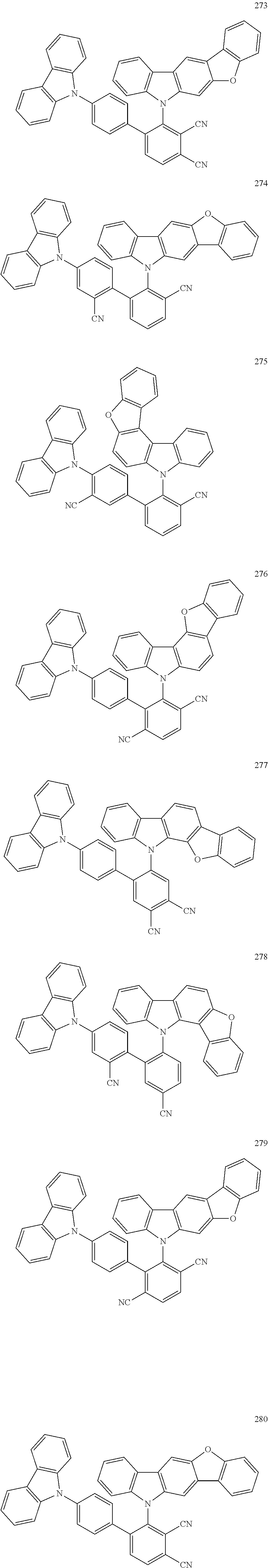

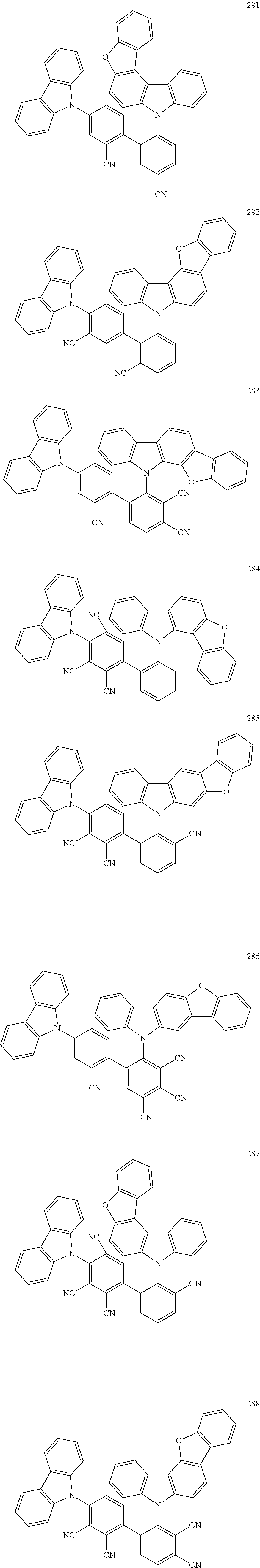

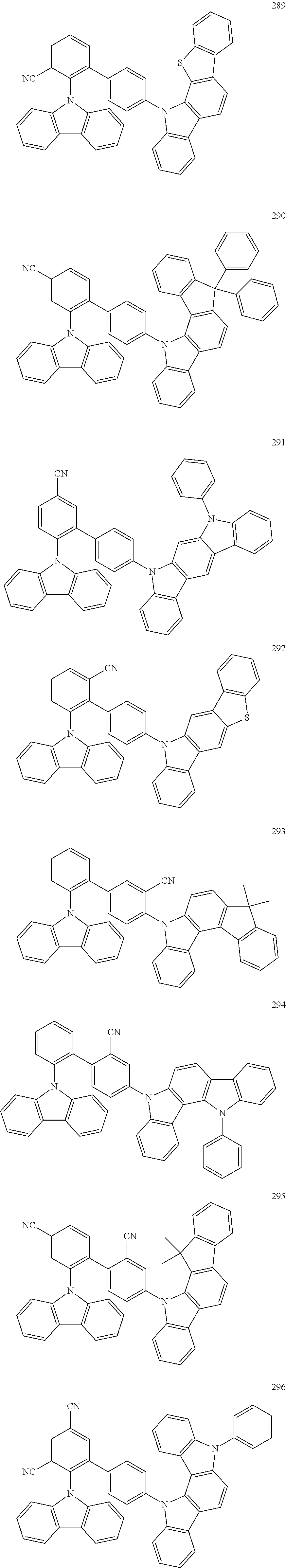

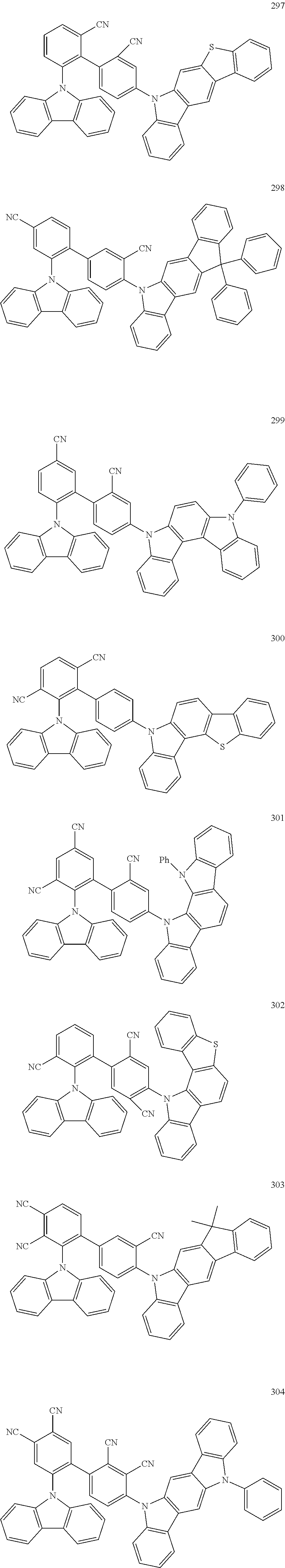

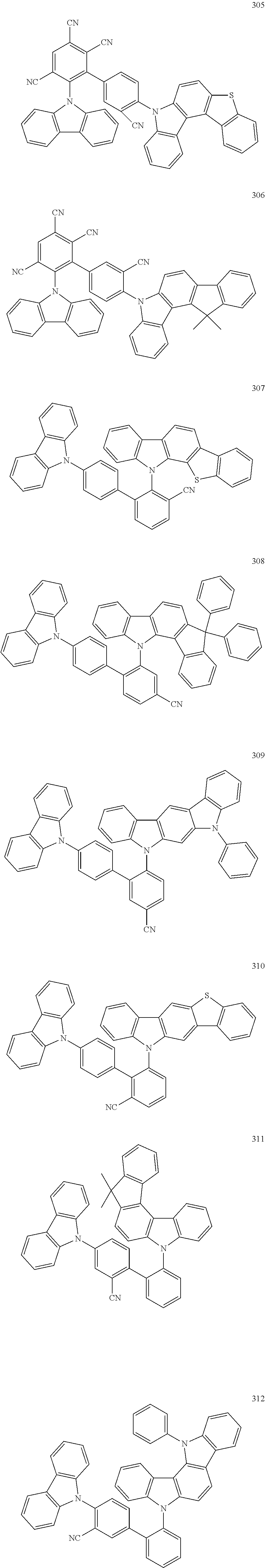

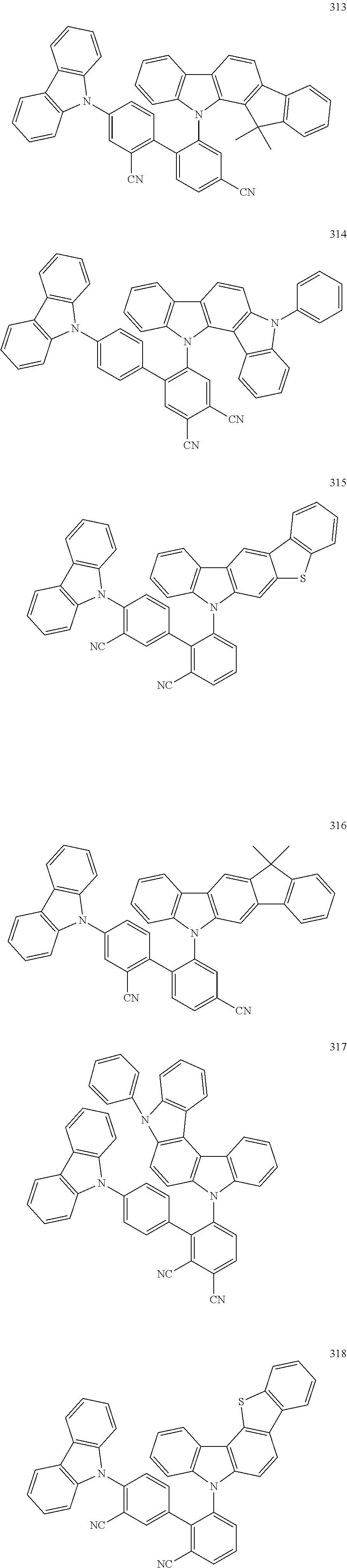

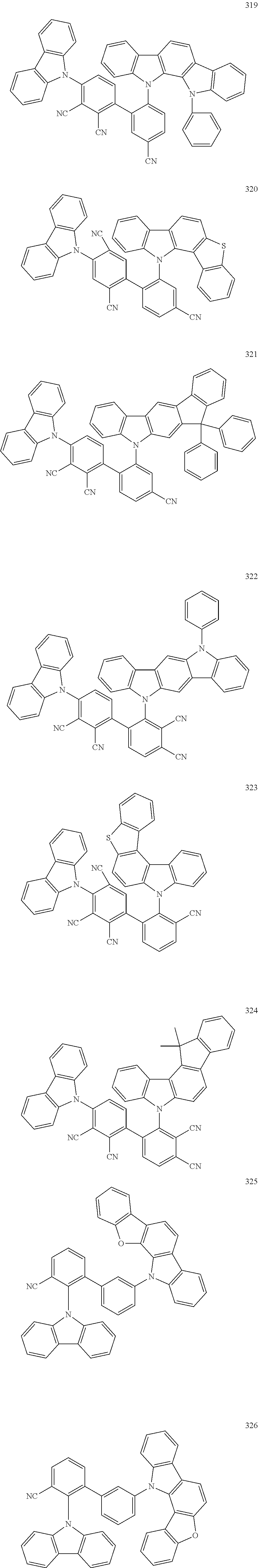

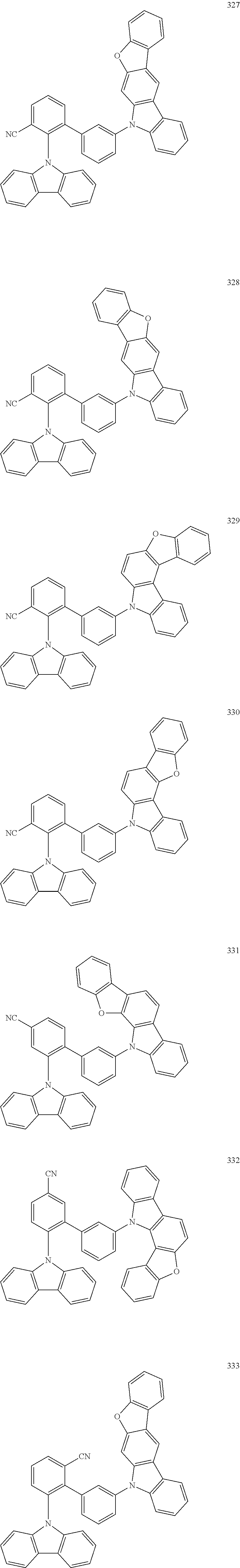

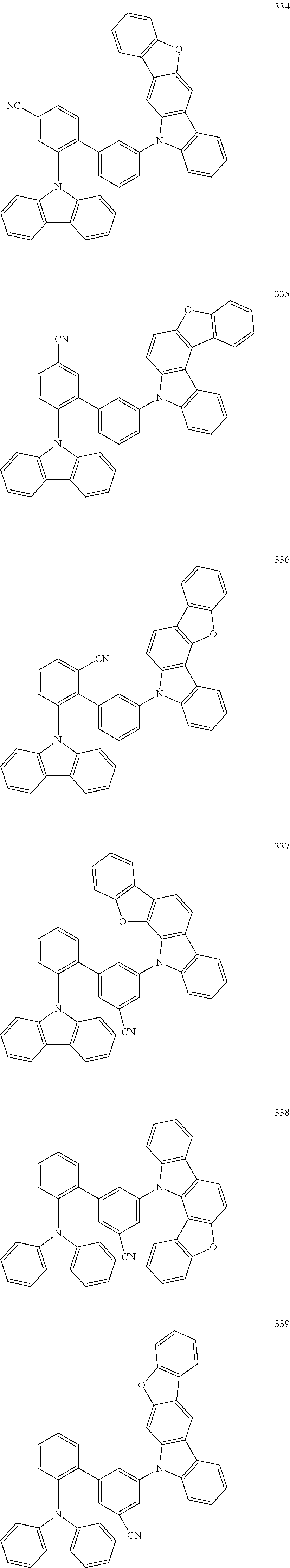 [SMILES: N#Cc1ccc(-c2cccc(-n3c4ccccc4c4cc5oc6ccccc6c5cc43)c2)c(-n2c3ccccc3c3ccccc32)c1 ; N#Cc1ccc(-n2c3ccccc3c3ccccc32)c(-c2cccc(-n3c4ccccc4c4c5c(ccc43)oc3ccccc35)c2)c1 ; N#Cc1cccc(-n2c3ccccc3c3ccccc32)c1-c1cccc(-n2c3ccccc3c3c4oc5ccccc5c4ccc32)c1 ; N#Cc1cc(-c2ccccc2-n2c3ccccc3c3ccccc32)cc(-n2c3ccccc3c3ccc4c5ccccc5oc4c32)c1 ; N#Cc1cc(-c2ccccc2-n2c3ccccc3c3ccccc32)cc(-n2c3ccccc3c3ccc4oc5ccccc5c4c32)c1 ; N#Cc1cc(-c2ccccc2-n2c3ccccc3c3ccccc32)cc(-n2c3ccccc3c3cc4c(cc32)oc2ccccc24)c1]

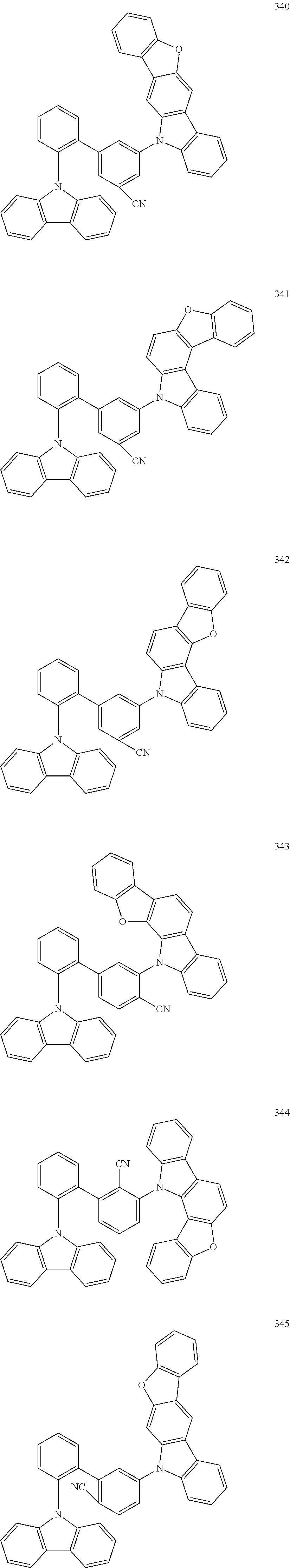 [SMILES: N#Cc1cc(-c2ccccc2-n2c3ccccc3c3ccccc32)cc(-n2c3ccccc3c3cc4oc5ccccc5c4cc32)c1 ; N#Cc1cc(-c2ccccc2-n2c3ccccc3c3ccccc32)cc(-n2c3ccccc3c3c4c(ccc32)oc2ccccc24)c1 ; N#Cc1cc(-c2ccccc2-n2c3ccccc3c3ccccc32)cc(-n2c3ccccc3c3c4oc5ccccc5c4ccc32)c1 ; N#Cc1ccc(-c2ccccc2-n2c3ccccc3c3ccccc32)cc1-n1c2ccccc2c2ccc3c4ccccc4oc3c21 ; N#Cc1c(-c2ccccc2-n2c3ccccc3c3ccccc32)cccc1-n1c2ccccc2c2ccc3oc4ccccc4c3c21 ; N#Cc1ccc(-n2c3ccccc3c3cc4c(cc32)oc2ccccc24)cc1-c1ccccc1-n1c2ccccc2c2ccccc21]

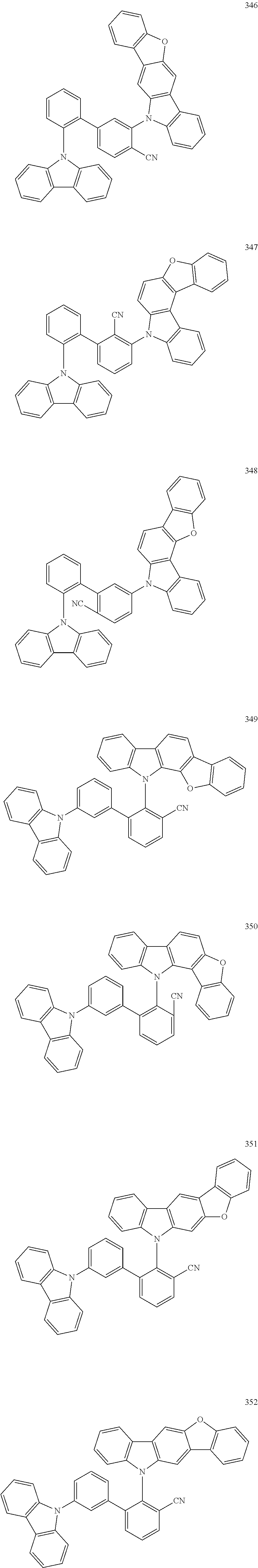

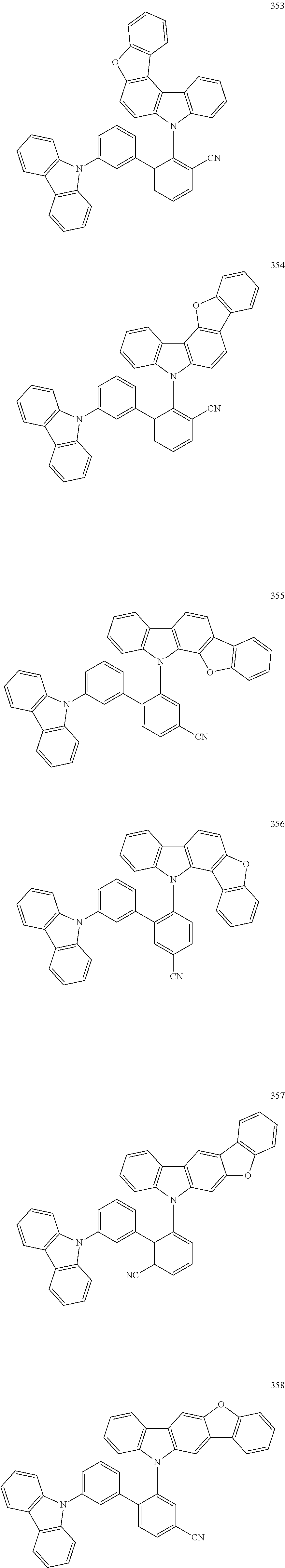 [SMILES: N#Cc1cccc(-c2cccc(-n3c4ccccc4c4ccccc43)c2)c1-n1c2ccccc2c2c3c(ccc21)oc1ccccc13 ; N#Cc1cccc(-c2cccc(-n3c4ccccc4c4ccccc43)c2)c1-n1c2ccccc2c2c3oc4ccccc4c3ccc21 ; N#Cc1ccc(-c2cccc(-n3c4ccccc4c4ccccc43)c2)c(-n2c3ccccc3c3ccc4c5ccccc5oc4c32)c1 ; N#Cc1ccc(-n2c3ccccc3c3ccc4oc5ccccc5c4c32)c(-c2cccc(-n3c4ccccc4c4ccccc43)c2)c1 ; N#Cc1cccc(-n2c3ccccc3c3cc4c(cc32)oc2ccccc24)c1-c1cccc(-n2c3ccccc3c3ccccc32)c1 ; N#Cc1ccc(-c2cccc(-n3c4ccccc4c4ccccc43)c2)c(-n2c3ccccc3c3cc4oc5ccccc5c4cc32)c1]

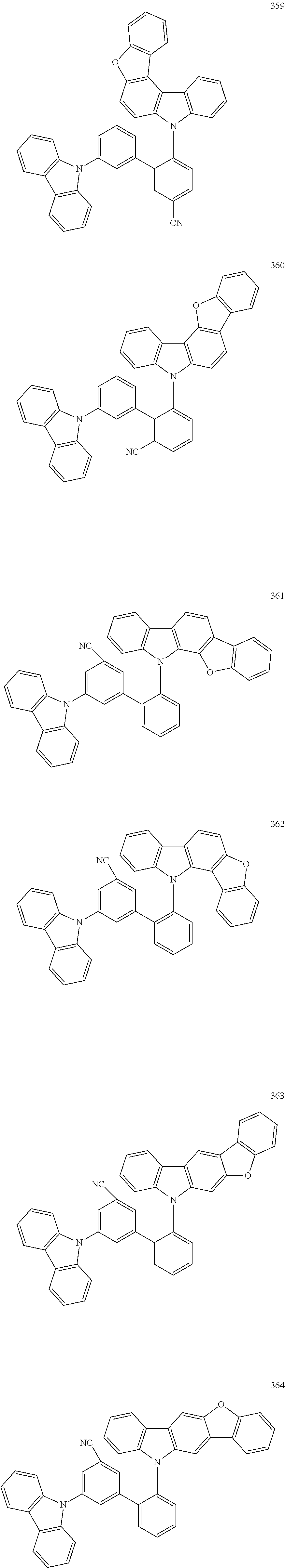 [SMILES: N#Cc1ccc(-n2c3ccccc3c3c4c(ccc32)oc2ccccc24)c(-c2cccc(-n3c4ccccc4c4ccccc43)c2)c1 ; N#Cc1cccc(-n2c3ccccc3c3c4oc5ccccc5c4ccc32)c1-c1cccc(-n2c3ccccc3c3ccccc32)c1 ; N#Cc1cc(-c2ccccc2-n2c3ccccc3c3ccc4c5ccccc5oc4c32)cc(-n2c3ccccc3c3ccccc32)c1 ; N#Cc1cc(-c2ccccc2-n2c3ccccc3c3ccc4oc5ccccc5c4c32)cc(-n2c3ccccc3c3ccccc32)c1 ; N#Cc1cc(-c2ccccc2-n2c3ccccc3c3cc4c(cc32)oc2ccccc24)cc(-n2c3ccccc3c3ccccc32)c1 ; N#Cc1cc(-c2ccccc2-n2c3ccccc3c3cc4oc5ccccc5c4cc32)cc(-n2c3ccccc3c3ccccc32)c1]

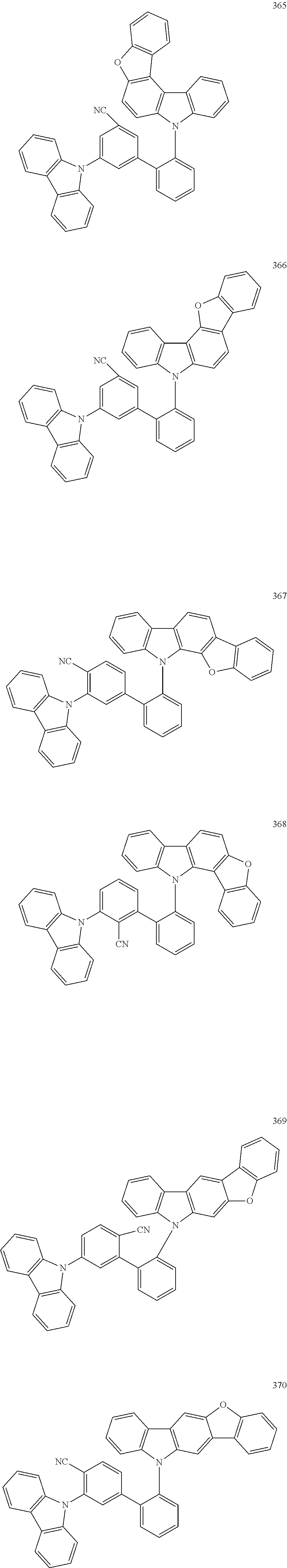 [SMILES: N#Cc1cc(-c2ccccc2-n2c3ccccc3c3c4c(ccc32)oc2ccccc24)cc(-n2c3ccccc3c3ccccc32)c1 ; N#Cc1cc(-c2ccccc2-n2c3ccccc3c3c4oc5ccccc5c4ccc32)cc(-n2c3ccccc3c3ccccc32)c1 ; N#Cc1ccc(-c2ccccc2-n2c3ccccc3c3ccc4c5ccccc5oc4c32)cc1-n1c2ccccc2c2ccccc21 ; N#Cc1c(-c2ccccc2-n2c3ccccc3c3ccc4oc5ccccc5c4c32)cccc1-n1c2ccccc2c2ccccc21 ; N#Cc1ccc(-n2c3ccccc3c3ccccc32)cc1-c1ccccc1-n1c2ccccc2c2cc3c(cc21)oc1ccccc13 ; N#Cc1ccc(-c2ccccc2-n2c3ccccc3c3cc4oc5ccccc5c4cc32)cc1-n1c2ccccc2c2ccccc21]

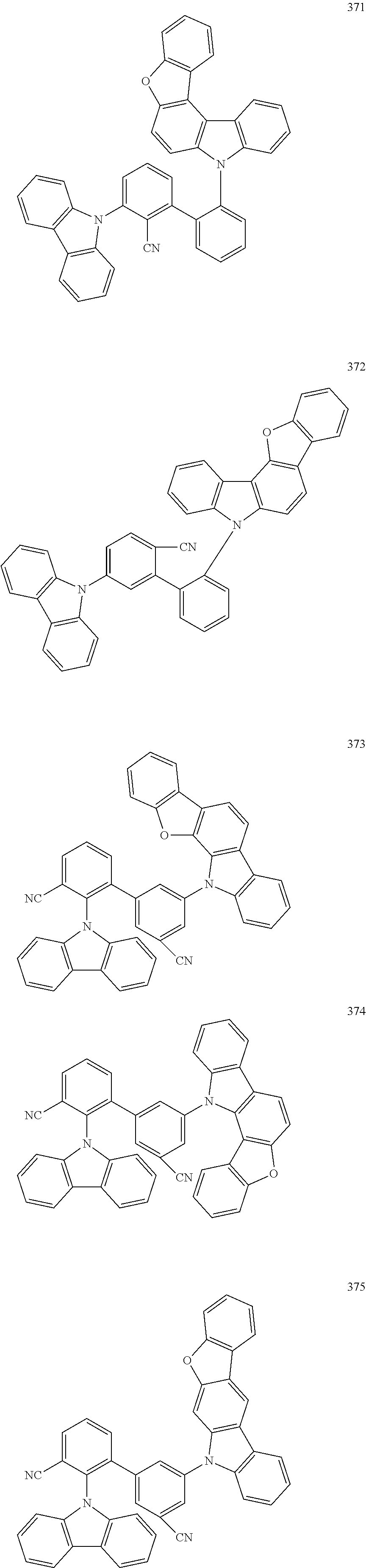 [SMILES: N#Cc1c(-c2ccccc2-n2c3ccccc3c3c4c(ccc32)oc2ccccc24)cccc1-n1c2ccccc2c2ccccc21 ; N#Cc1ccc(-n2c3ccccc3c3ccccc32)cc1-c1ccccc1-n1c2ccccc2c2c3oc4ccccc4c3ccc21 ; N#Cc1cc(-c2cccc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)cc(-n2c3ccccc3c3ccc4oc5ccccc5c4c32)c1 ; N#Cc1cc(-c2cccc(C#N)c2-n2c3ccccc3c3ccccc32)cc(-n2c3ccccc3c3cc4c(cc32)oc2ccccc24)c1]

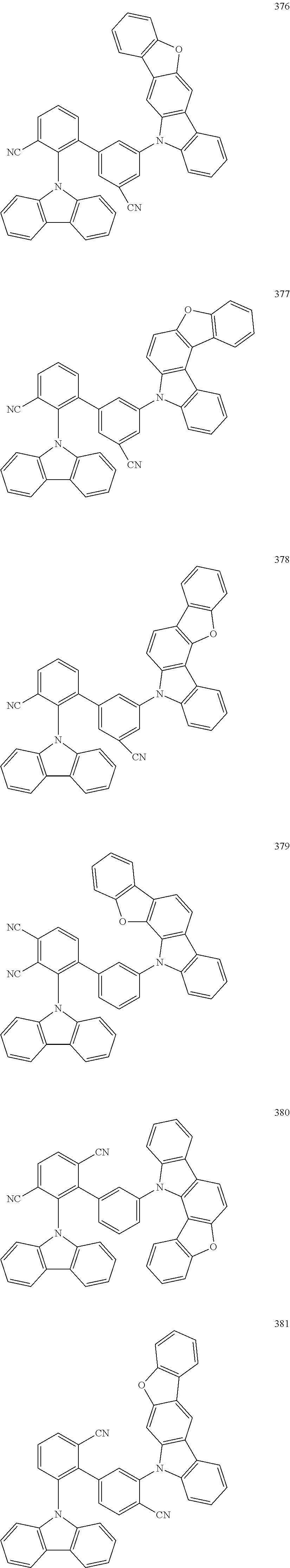 [SMILES: N#Cc1cc(-c2cccc(C#N)c2-n2c3ccccc3c3ccccc32)cc(-n2c3ccccc3c3cc4oc5ccccc5c4cc32)c1 ; N#Cc1cc(-c2cccc(C#N)c2-n2c3ccccc3c3ccccc32)cc(-n2c3ccccc3c3c4c(ccc32)oc2ccccc24)c1 ; N#Cc1cc(-c2cccc(C#N)c2-n2c3ccccc3c3ccccc32)cc(-n2c3ccccc3c3c4oc5ccccc5c4ccc32)c1 ; N#Cc1ccc(-c2cccc(-n3c4ccccc4c4ccc5c6ccccc6oc5c43)c2)c(-n2c3ccccc3c3ccccc32)c1C#N ; N#Cc1ccc(C#N)c(-n2c3ccccc3c3ccccc32)c1-c1cccc(-n2c3ccccc3c3ccc4oc5ccccc5c4c32)c1 ; N#Cc1ccc(-c2c(C#N)cccc2-n2c3ccccc3c3ccccc32)cc1-n1c2ccccc2c2cc3c(cc21)oc1ccccc13]

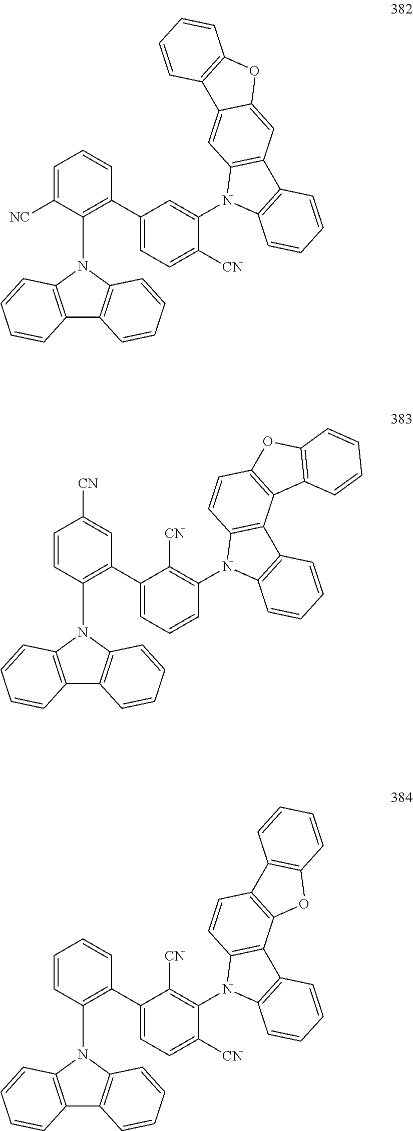 [SMILES: N#Cc1ccc(-c2cccc(C#N)c2-n2c3ccccc3c3ccccc32)cc1-n1c2ccccc2c2cc3oc4ccccc4c3cc21 ; N#Cc1ccc(-n2c3ccccc3c3ccccc32)c(-c2cccc(-n3c4ccccc4c4c5c(ccc43)oc3ccccc35)c2C#N)c1 ; N#Cc1ccc(-c2ccccc2-n2c3ccccc3c3ccccc32)c(C#N)c1-n1c2ccccc2c2c3oc4ccccc4c3ccc21]

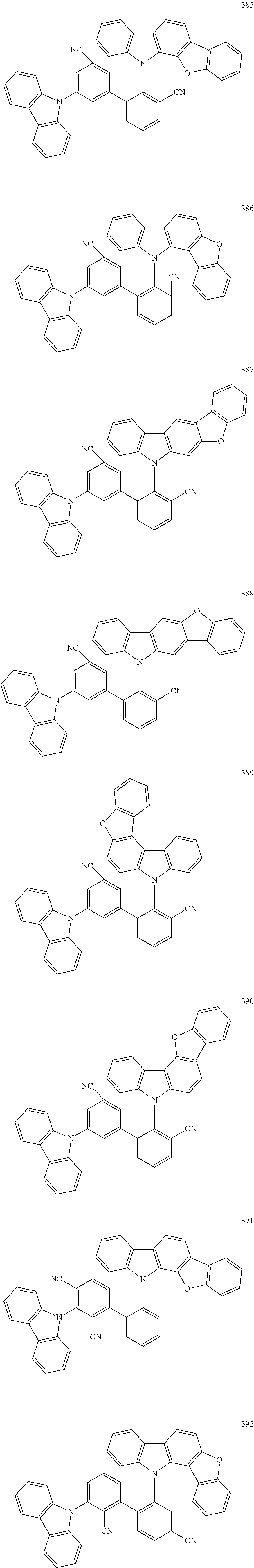

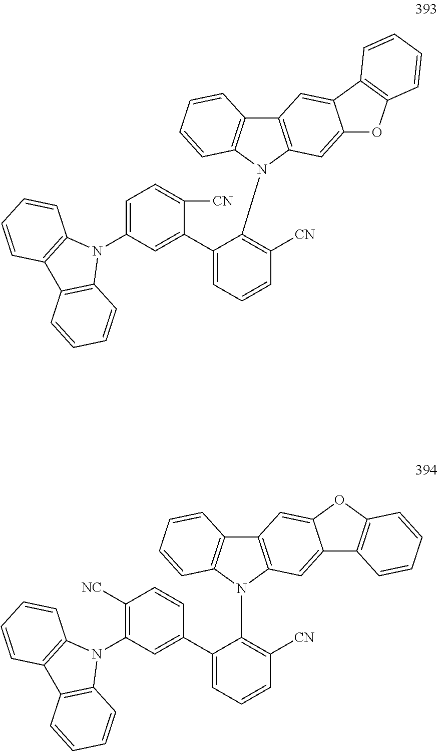 [SMILES: N#Cc1ccc(-n2c3ccccc3c3ccccc32)cc1-c1cccc(C#N)c1-n1c2ccccc2c2cc3c(cc21)oc1ccccc13 ; N#Cc1ccc(-c2cccc(C#N)c2-n2c3ccccc3c3cc4oc5ccccc5c4cc32)cc1-n1c2ccccc2c2ccccc21]

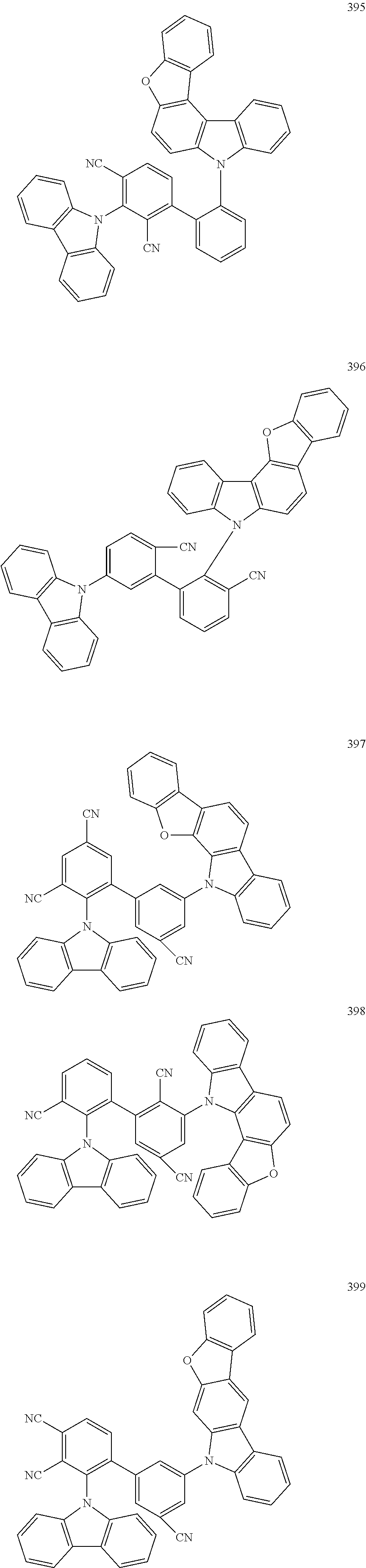 [SMILES: N#Cc1ccc(-c2ccccc2-n2c3ccccc3c3c4c(ccc32)oc2ccccc24)c(C#N)c1-n1c2ccccc2c2ccccc21 ; N#Cc1ccc(-n2c3ccccc3c3ccccc32)cc1-c1cccc(C#N)c1-n1c2ccccc2c2c3oc4ccccc4c3ccc21 ; N#Cc1cc(-c2cc(C#N)cc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)c(C#N)c(-n2c3ccccc3c3ccc4oc5ccccc5c4c32)c1 ; N#Cc1cc(-c2ccc(C#N)c(C#N)c2-n2c3ccccc3c3ccccc32)cc(-n2c3ccccc3c3cc4c(cc32)oc2ccccc24)c1]

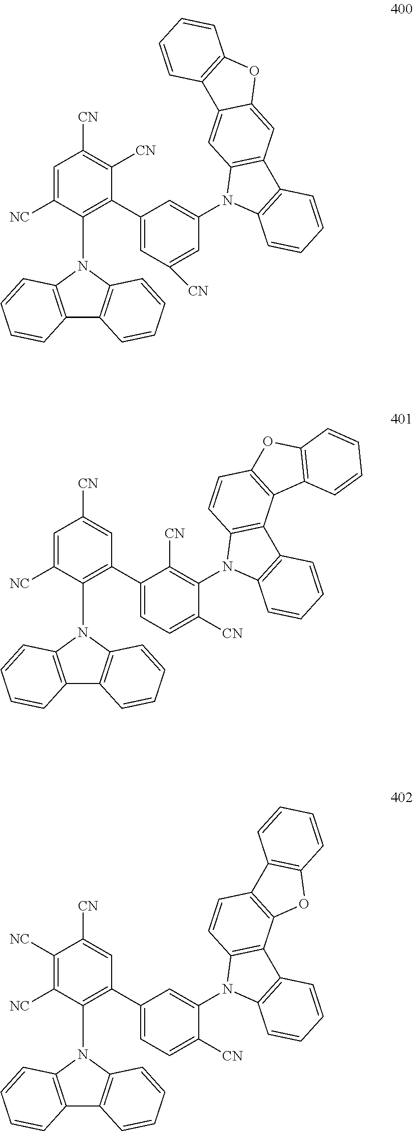 [SMILES: N#Cc1cc(-c2c(C#N)c(C#N)cc(C#N)c2-n2c3ccccc3c3ccccc32)cc(-n2c3ccccc3c3cc4oc5ccccc5c4cc32)c1 ; N#Cc1cc(C#N)c(-n2c3ccccc3c3ccccc32)c(-c2ccc(C#N)c(-n3c4ccccc4c4c5c(ccc43)oc3ccccc35)c2C#N)c1 ; N#Cc1ccc(-c2cc(C#N)c(C#N)c(C#N)c2-n2c3ccccc3c3ccccc32)cc1-n1c2ccccc2c2c3oc4ccccc4c3ccc21]

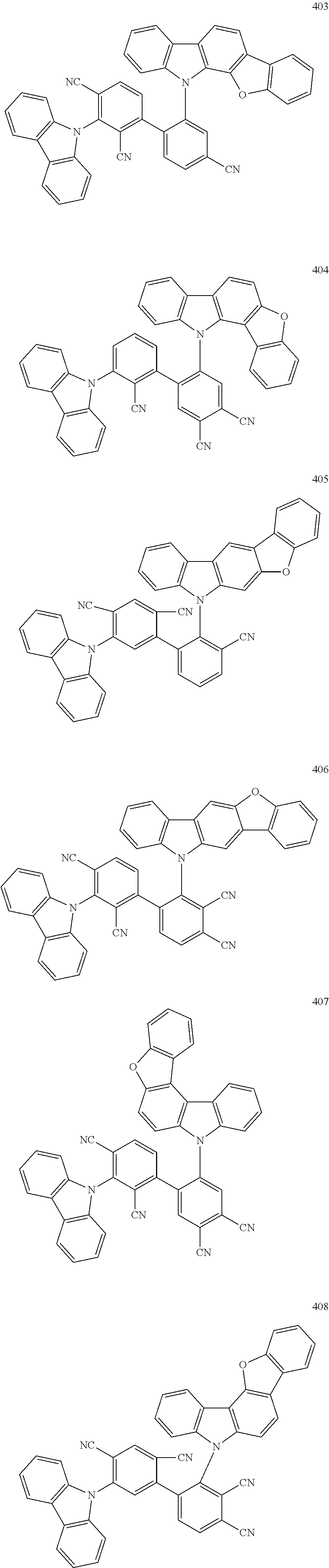

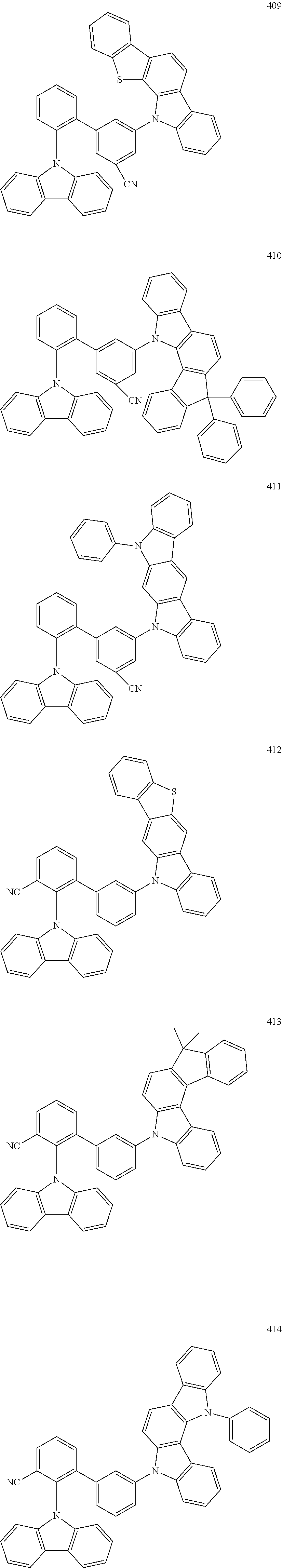

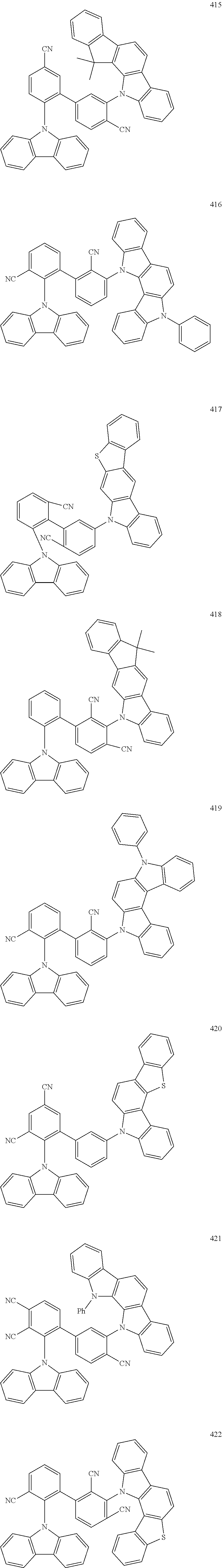

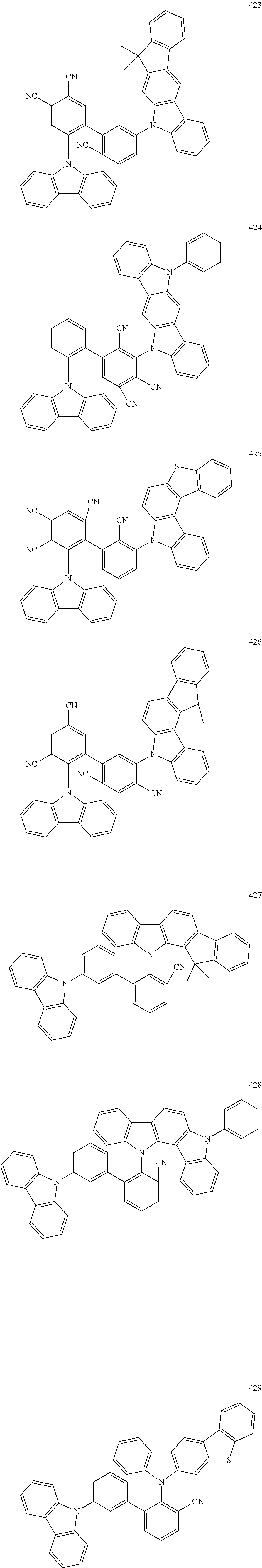

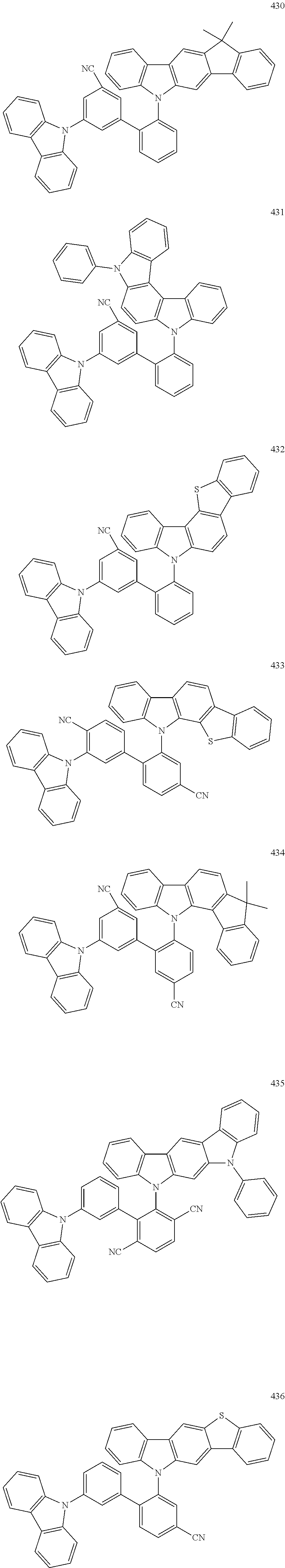

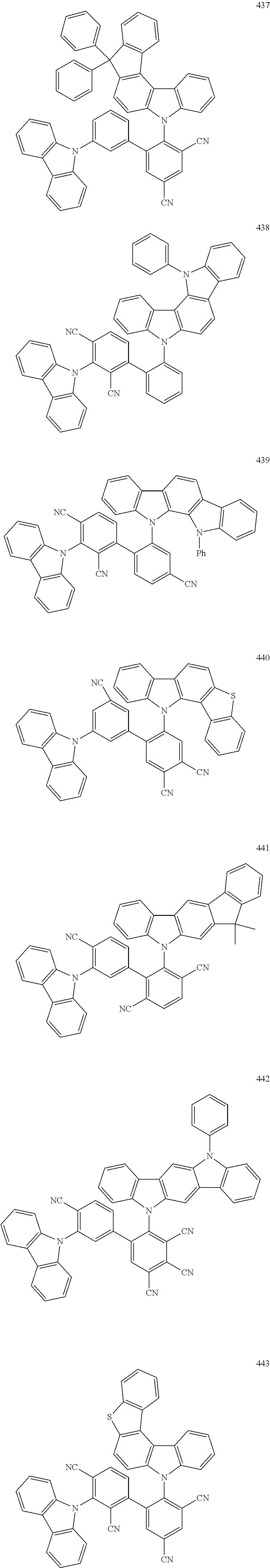

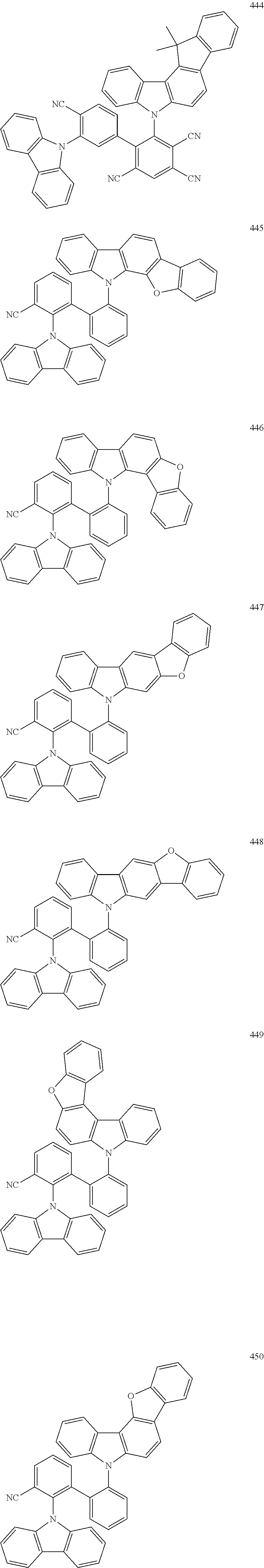

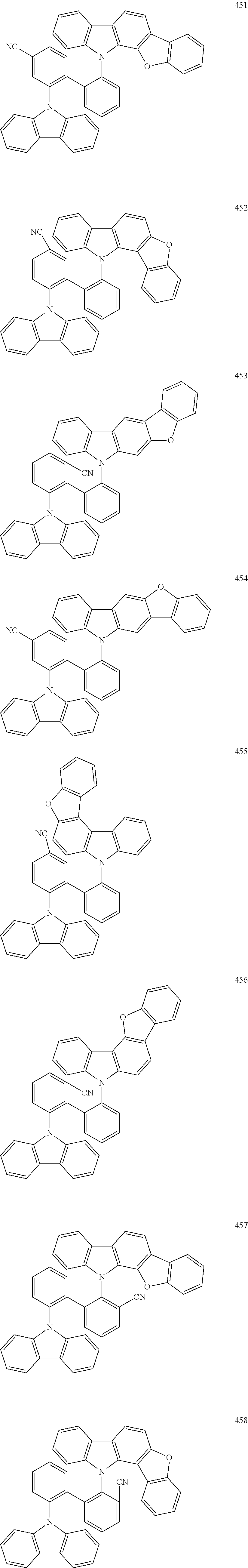

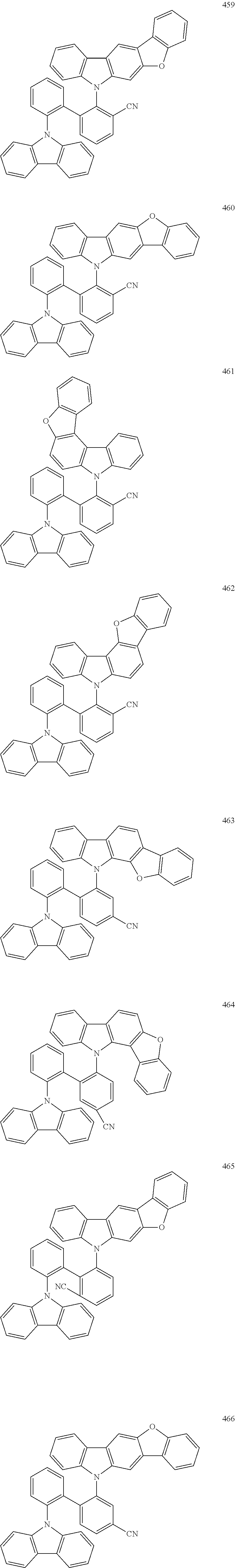

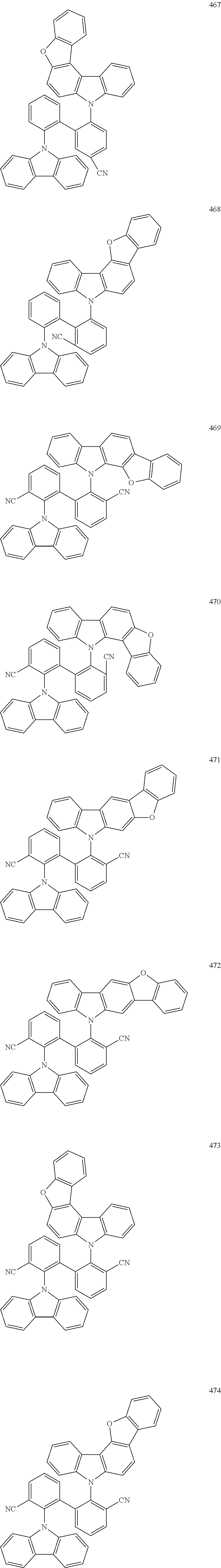

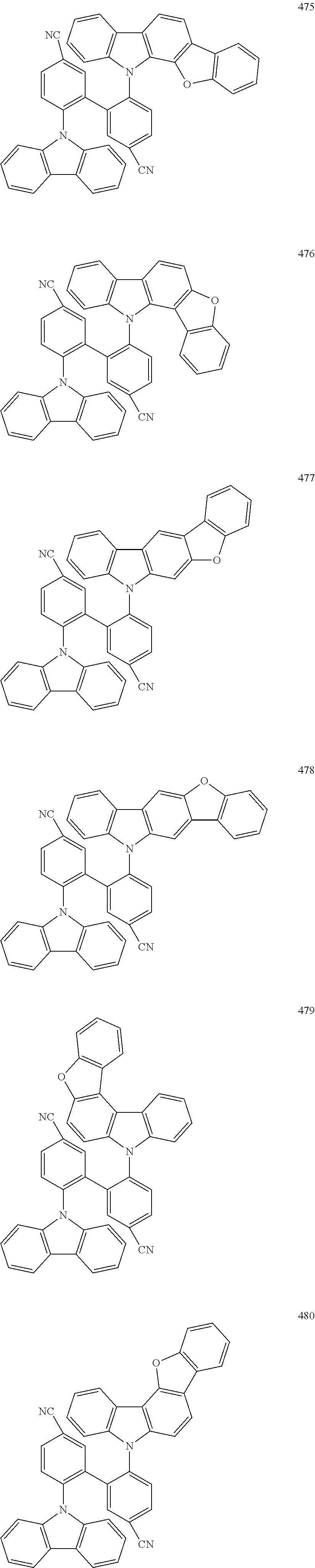 [SMILES: N#Cc1ccc(-n2c3ccccc3c3ccccc32)c(-c2cc(C#N)ccc2-n2c3ccccc3c3ccc4c5ccccc5oc4c32)c1 ; N#Cc1ccc(-n2c3ccccc3c3ccccc32)c(-c2cc(C#N)ccc2-n2c3ccccc3c3ccc4oc5ccccc5c4c32)c1 ; N#Cc1ccc(-n2c3ccccc3c3ccccc32)c(-c2cc(C#N)ccc2-n2c3ccccc3c3cc4c(cc32)oc2ccccc24)c1 ; N#Cc1ccc(-n2c3ccccc3c3ccccc32)c(-c2cc(C#N)ccc2-n2c3ccccc3c3cc4oc5ccccc5c4cc32)c1 ; N#Cc1ccc(-n2c3ccccc3c3ccccc32)c(-c2cc(C#N)ccc2-n2c3ccccc3c3c4c(ccc32)oc2ccccc24)c1 ; N#Cc1ccc(-n2c3ccccc3c3ccccc32)c(-c2cc(C#N)ccc2-n2c3ccccc3c3c4oc5ccccc5c4ccc32)c1]

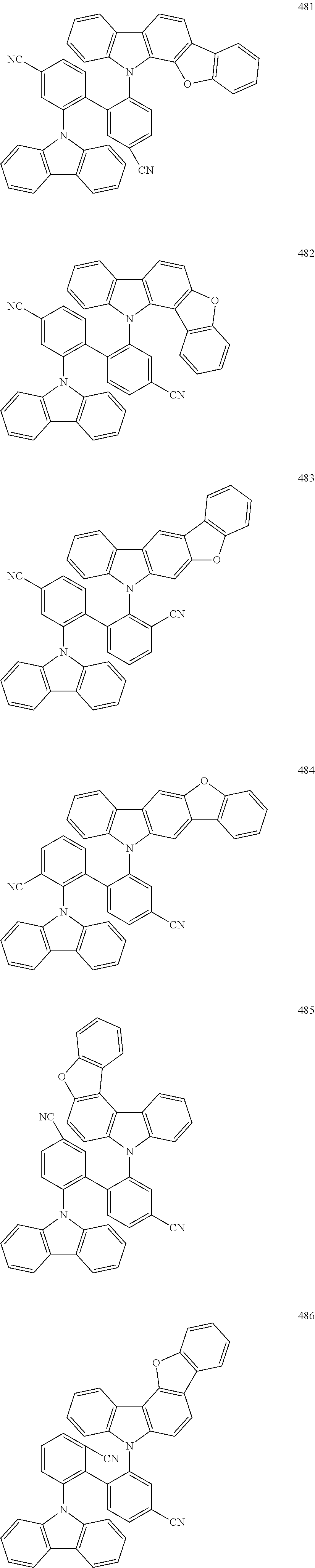 [SMILES: N#Cc1ccc(-n2c3ccccc3c3ccc4c5ccccc5oc4c32)c(-c2ccc(C#N)cc2-n2c3ccccc3c3ccccc32)c1 ; N#Cc1ccc(-c2ccc(C#N)cc2-n2c3ccccc3c3ccc4oc5ccccc5c4c32)c(-n2c3ccccc3c3ccccc32)c1 ; N#Cc1ccc(-c2cccc(C#N)c2-n2c3ccccc3c3cc4c(cc32)oc2ccccc24)c(-n2c3ccccc3c3ccccc32)c1 ; N#Cc1ccc(-c2cccc(C#N)c2-n2c3ccccc3c3ccccc32)c(-n2c3ccccc3c3cc4oc5ccccc5c4cc32)c1 ; N#Cc1ccc(-n2c3ccccc3c3ccccc32)c(-c2ccc(C#N)cc2-n2c3ccccc3c3c4c(ccc32)oc2ccccc24)c1 ; N#Cc1ccc(-c2c(C#N)cccc2-n2c3ccccc3c3ccccc32)c(-n2c3ccccc3c3c4oc5ccccc5c4ccc32)c1]

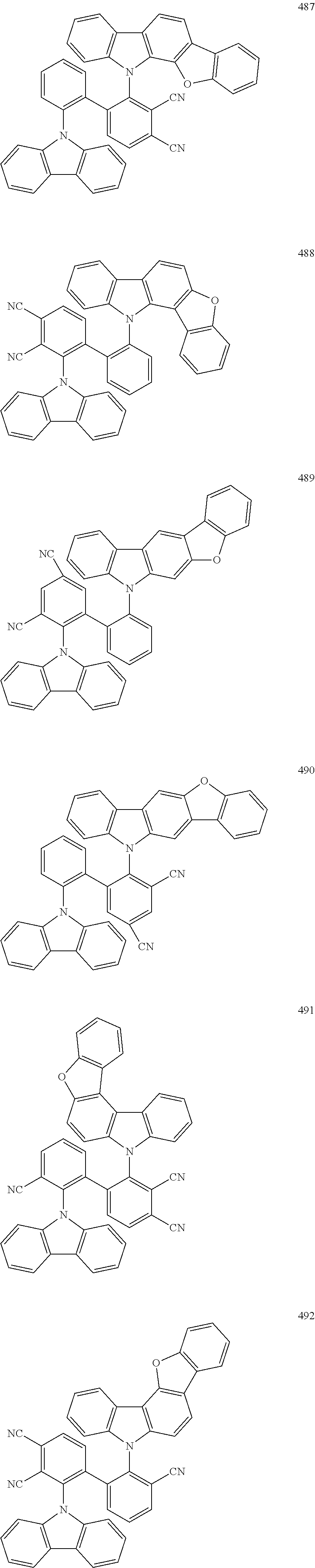

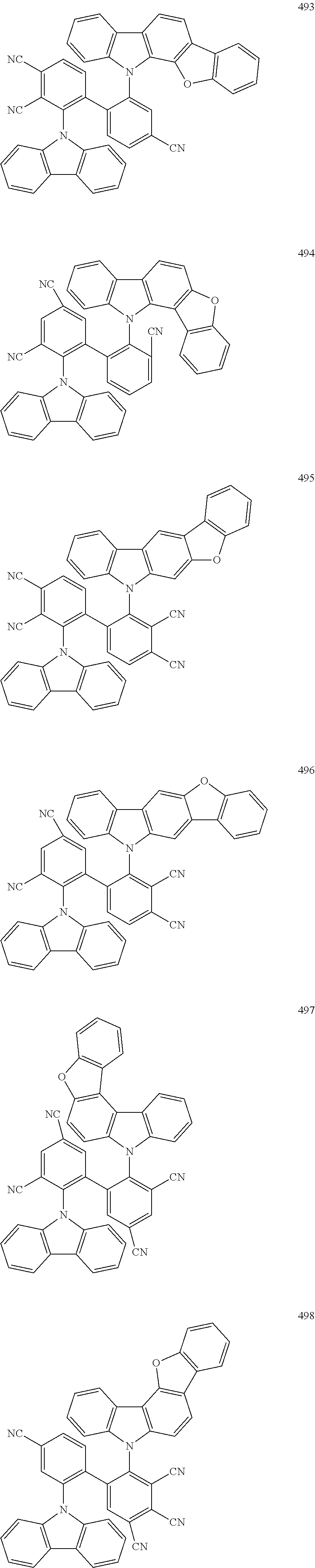

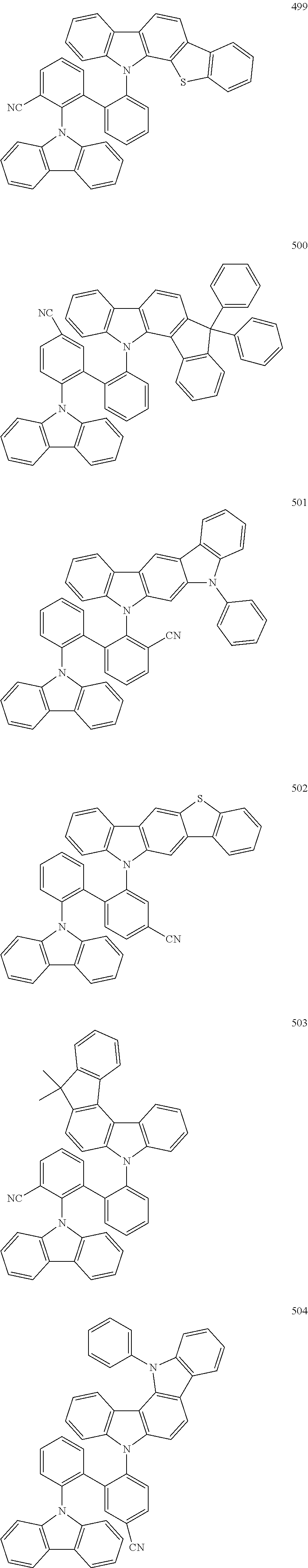

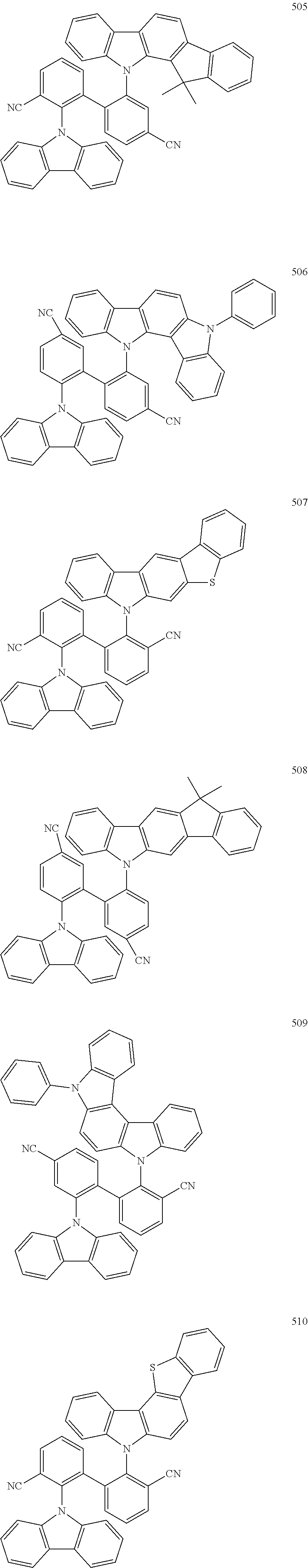

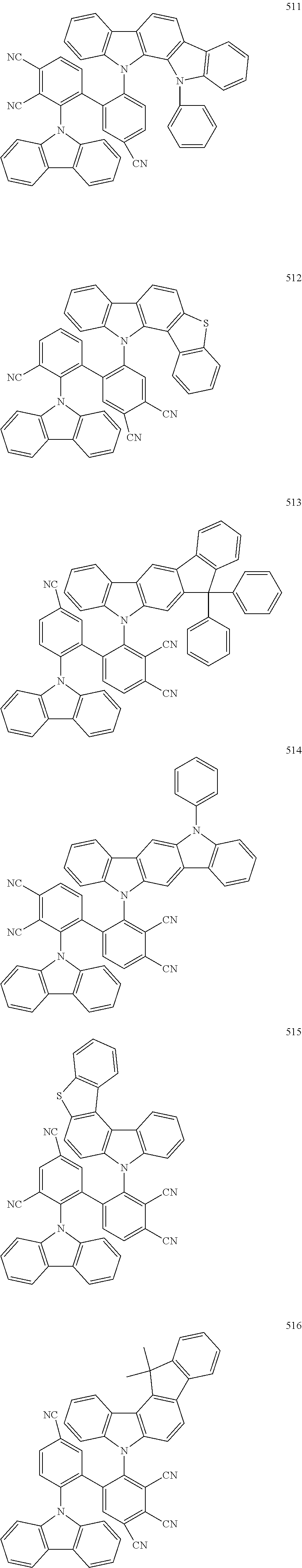

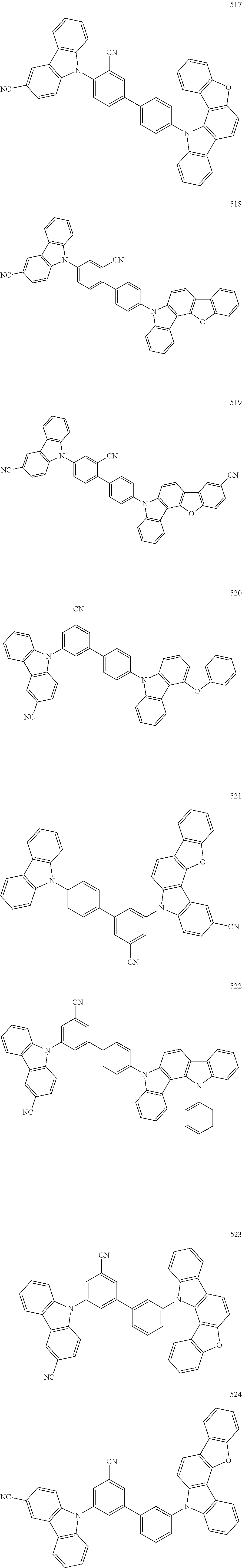

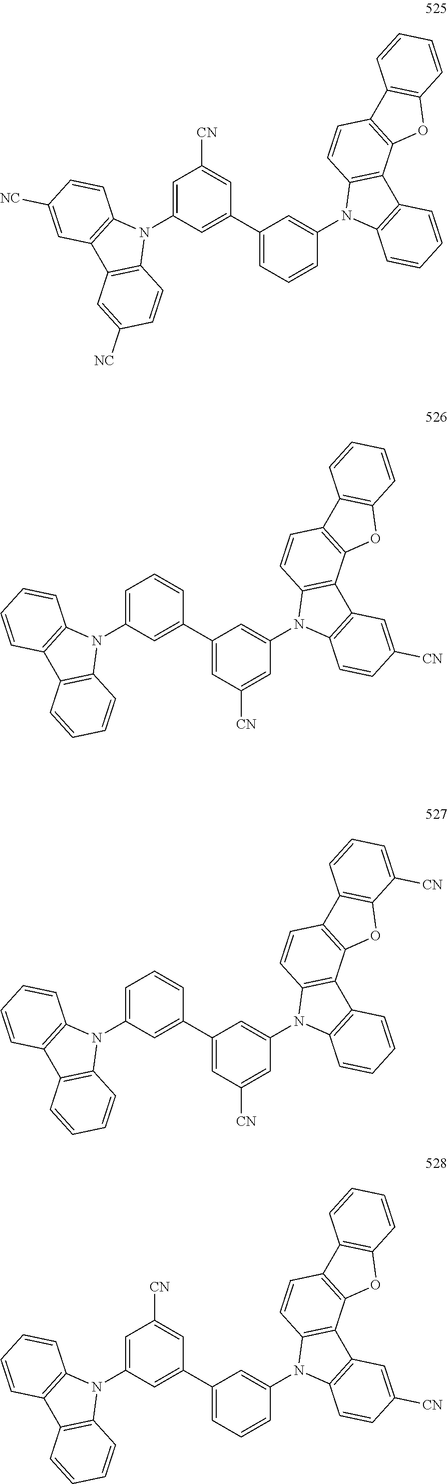 [SMILES: N#Cc1cc(-c2cccc(-n3c4ccccc4c4c5oc6ccccc6c5ccc43)c2)cc(-n2c3ccc(C#N)cc3c3cc(C#N)ccc32)c1 ; N#Cc1cc(-c2cccc(-n3c4ccccc4c4ccccc43)c2)cc(-n2c3ccc(C#N)cc3c3c4oc5ccccc5c4ccc32)c1 ; N#Cc1cc(-c2cccc(-n3c4ccccc4c4ccccc43)c2)cc(-n2c3ccccc3c3c4oc5c(C#N)cccc5c4ccc32)c1 ; N#Cc1cc(-c2cccc(-n3c4ccc(C#N)cc4c4c5oc6ccccc6c5ccc43)c2)cc(-n2c3ccccc3c3ccccc32)c1]

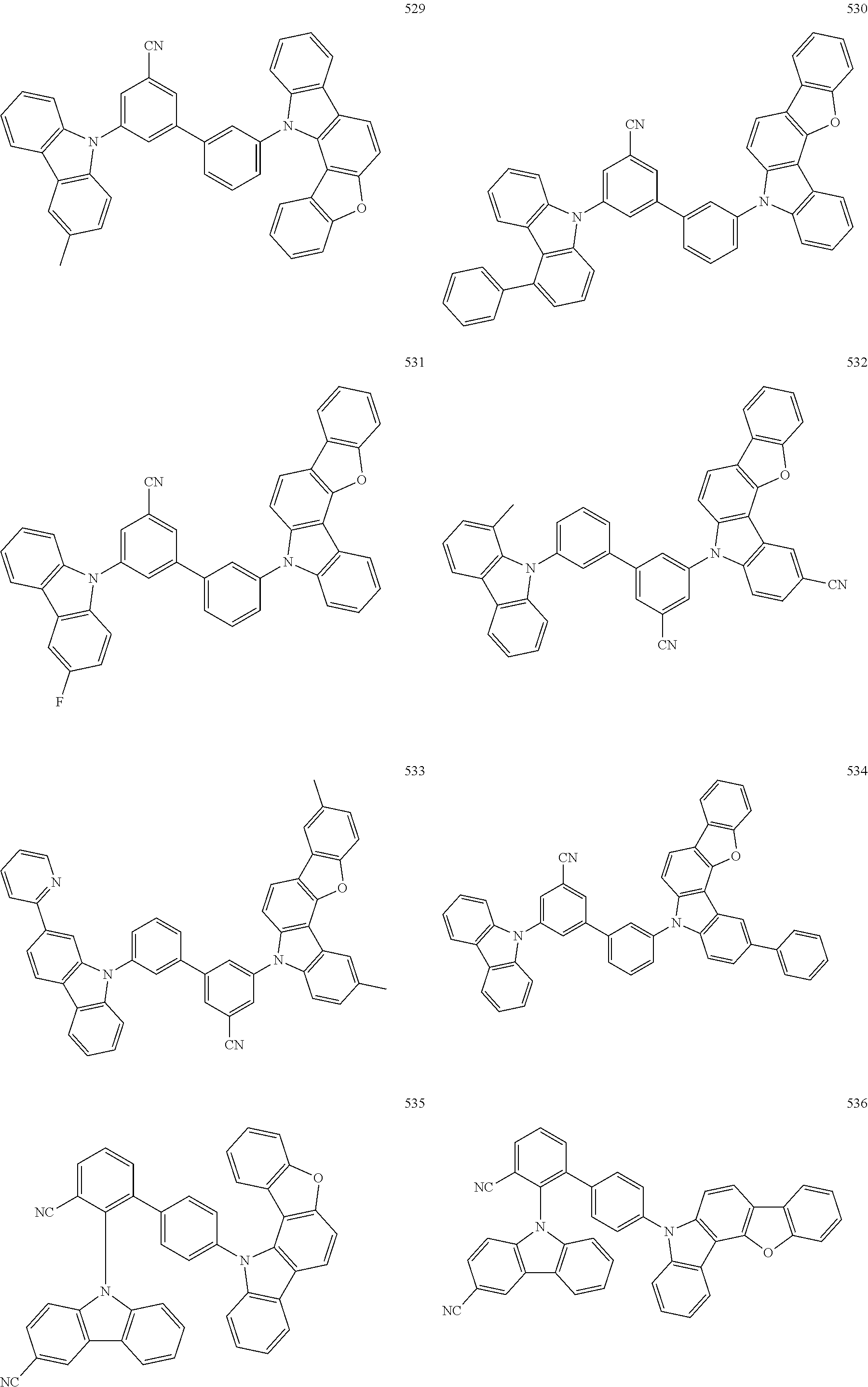

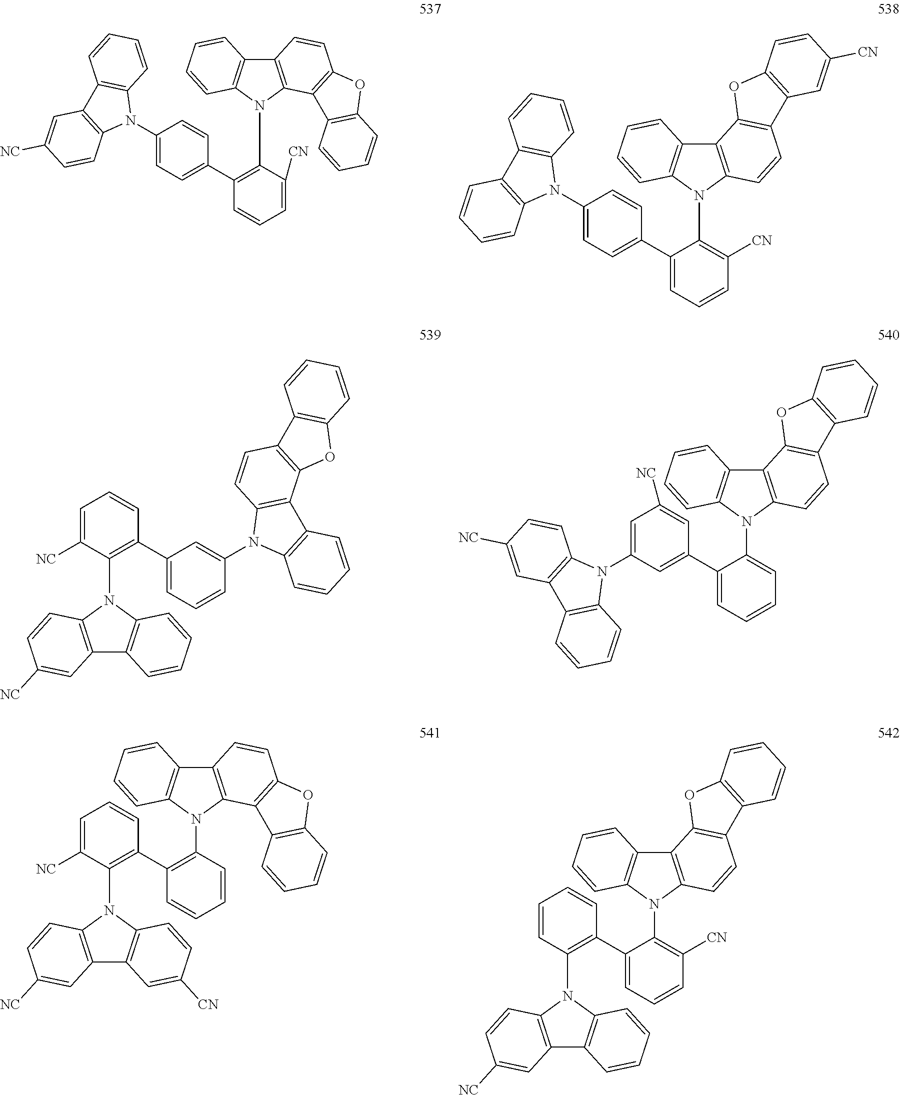 [SMILES: N#Cc1ccc2c(c1)c1ccccc1n2-c1ccc(-c2cccc(C#N)c2-n2c3ccccc3c3ccc4oc5ccccc5c4c32)cc1 ; N#Cc1ccc2oc3c(ccc4c3c3ccccc3n4-c3c(C#N)cccc3-c3ccc(-n4c5ccccc5c5ccccc54)cc3)c2c1 ; N#Cc1ccc2c(c1)c1ccccc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccccc3c3cc(C#N)ccc32)c1 ; N#Cc1ccc2c(c1)c1cc(C#N)ccc1n2-c1c(C#N)cccc1-c1ccccc1-n1c2ccccc2c2ccc3oc4ccccc4c3c21 ; N#Cc1ccc2c(c1)c1ccccc1n2-c1ccccc1-c1cccc(C#N)c1-n1c2ccccc2c2c3oc4ccccc4c3ccc21]

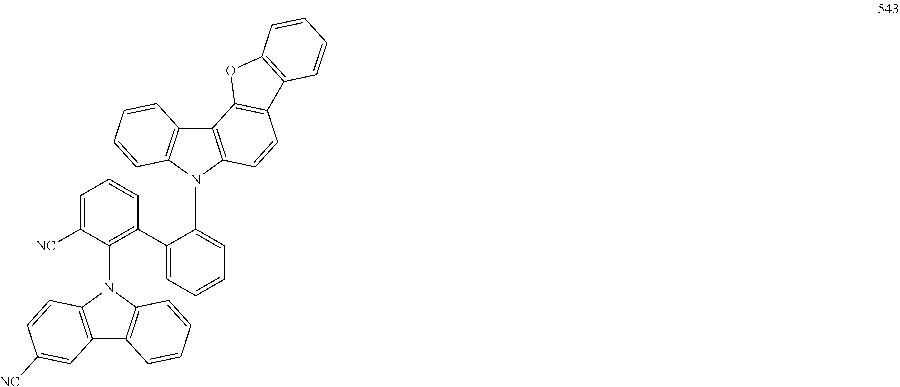 [SMILES: N#Cc1ccc2c(c1)c1ccccc1n2-c1c(C#N)cccc1-c1ccccc1-n1c2ccccc2c2c3oc4ccccc4c3ccc21]

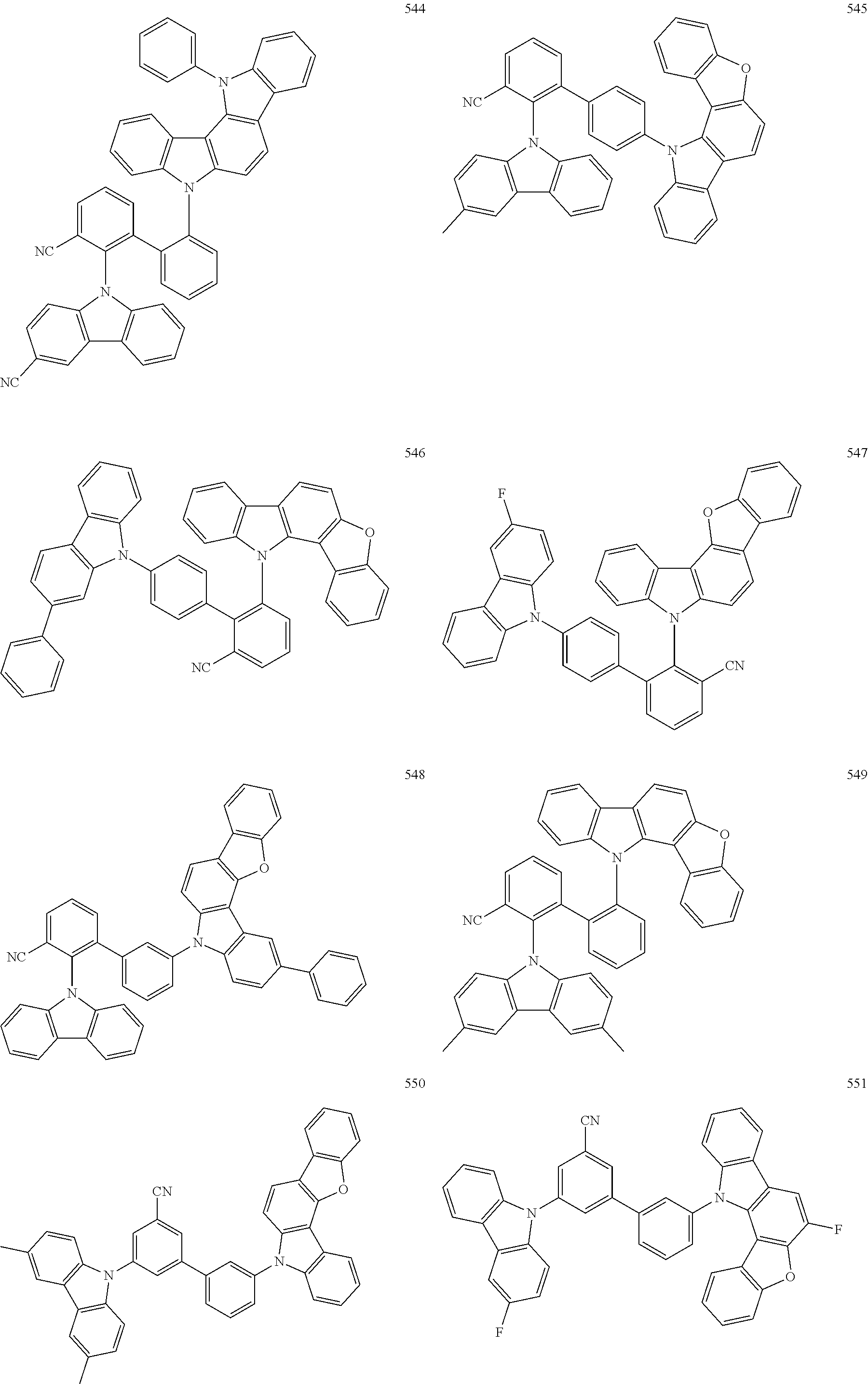

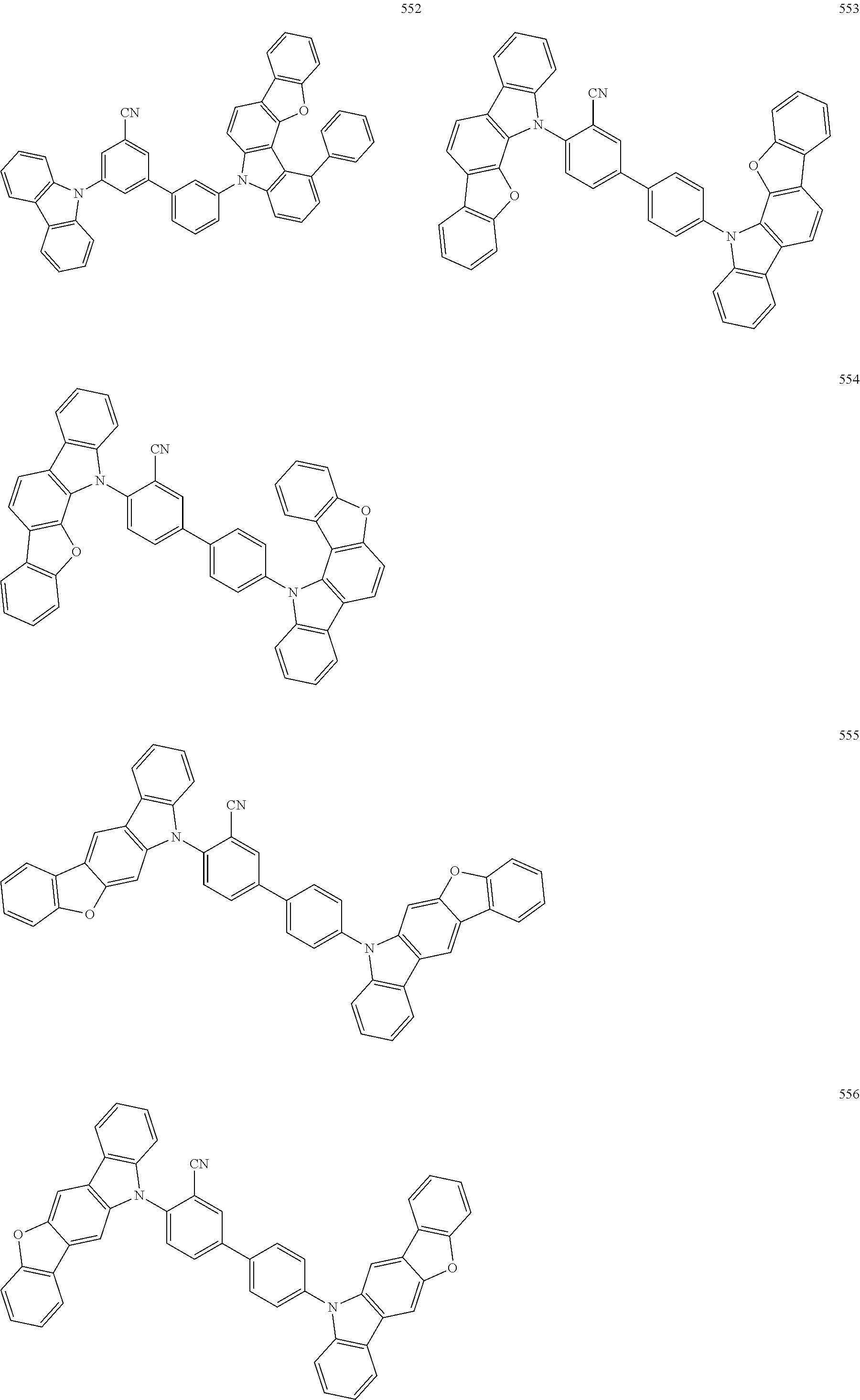 [SMILES: N#Cc1cc(-c2cccc(-n3c4cccc(-c5ccccc5)c4c4c5oc6ccccc6c5ccc43)c2)cc(-n2c3ccccc3c3ccccc32)c1 ; N#Cc1cc(-c2ccc(-n3c4ccccc4c4ccc5c6ccccc6oc5c43)cc2)ccc1-n1c2ccccc2c2ccc3c4ccccc4oc3c21 ; N#Cc1cc(-c2ccc(-n3c4ccccc4c4ccc5oc6ccccc6c5c43)cc2)ccc1-n1c2ccccc2c2ccc3c4ccccc4oc3c21 ; N#Cc1cc(-c2ccc(-n3c4ccccc4c4cc5c(cc43)oc3ccccc35)cc2)ccc1-n1c2ccccc2c2cc3c(cc21)oc1ccccc13 ; N#Cc1cc(-c2ccc(-n3c4ccccc4c4cc5oc6ccccc6c5cc43)cc2)ccc1-n1c2ccccc2c2cc3oc4ccccc4c3cc21]

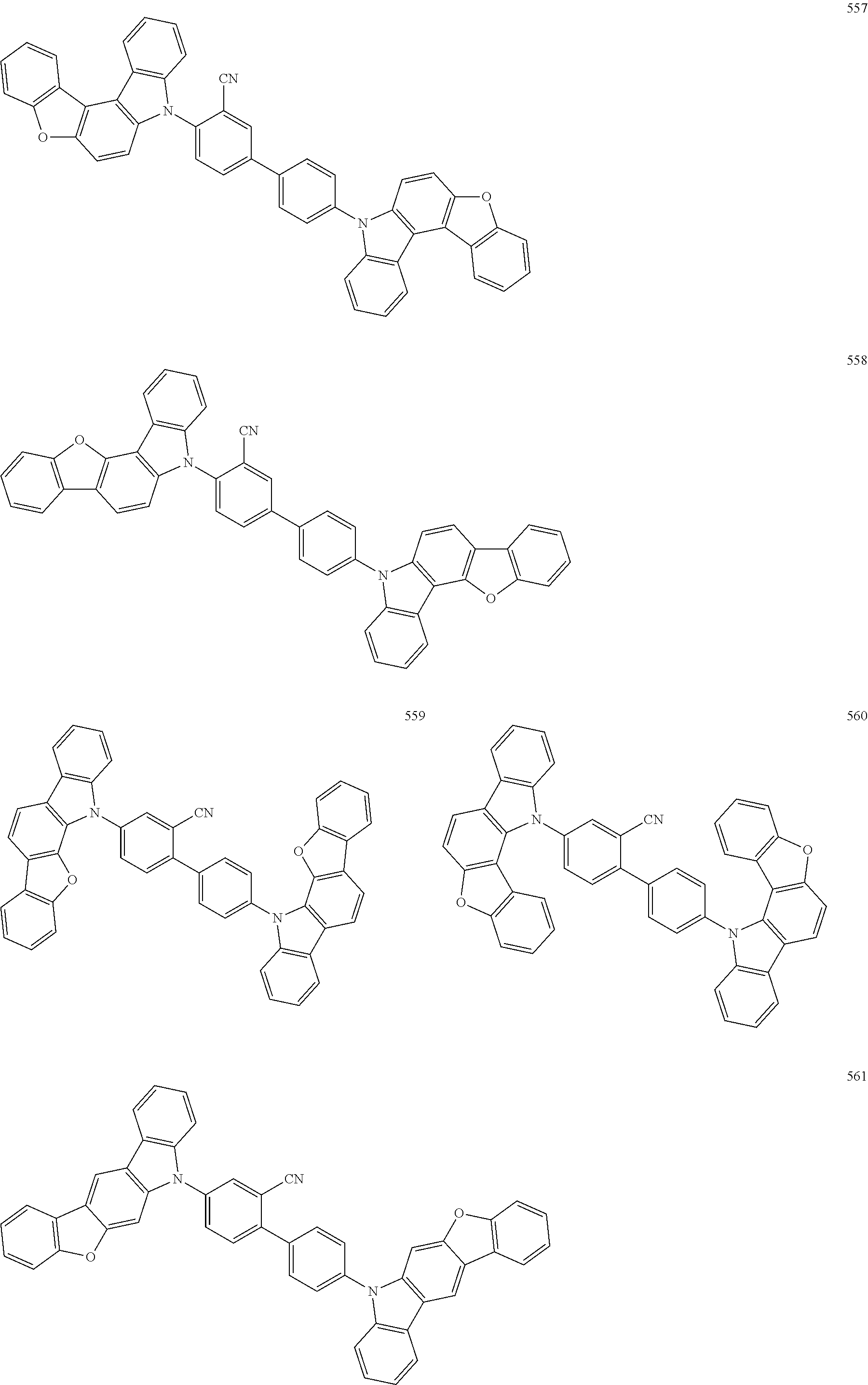 [SMILES: N#Cc1cc(-c2ccc(-n3c4ccccc4c4c5c(ccc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)cc2)ccc1-n1c2ccccc2c2c3oc4ccccc4c3ccc21 ; N#Cc1cc(-n2c3ccccc3c3ccc4c5ccccc5oc4c32)ccc1-c1ccc(-n2c3ccccc3c3ccc4c5ccccc5oc4c32)cc1 ; N#Cc1cc(-n2c3ccccc3c3ccc4oc5ccccc5c4c32)ccc1-c1ccc(-n2c3ccccc3c3ccc4oc5ccccc5c4c32)cc1 ; N#Cc1cc(-n2c3ccccc3c3cc4c(cc32)oc2ccccc24)ccc1-c1ccc(-n2c3ccccc3c3cc4c(cc32)oc2ccccc24)cc1]

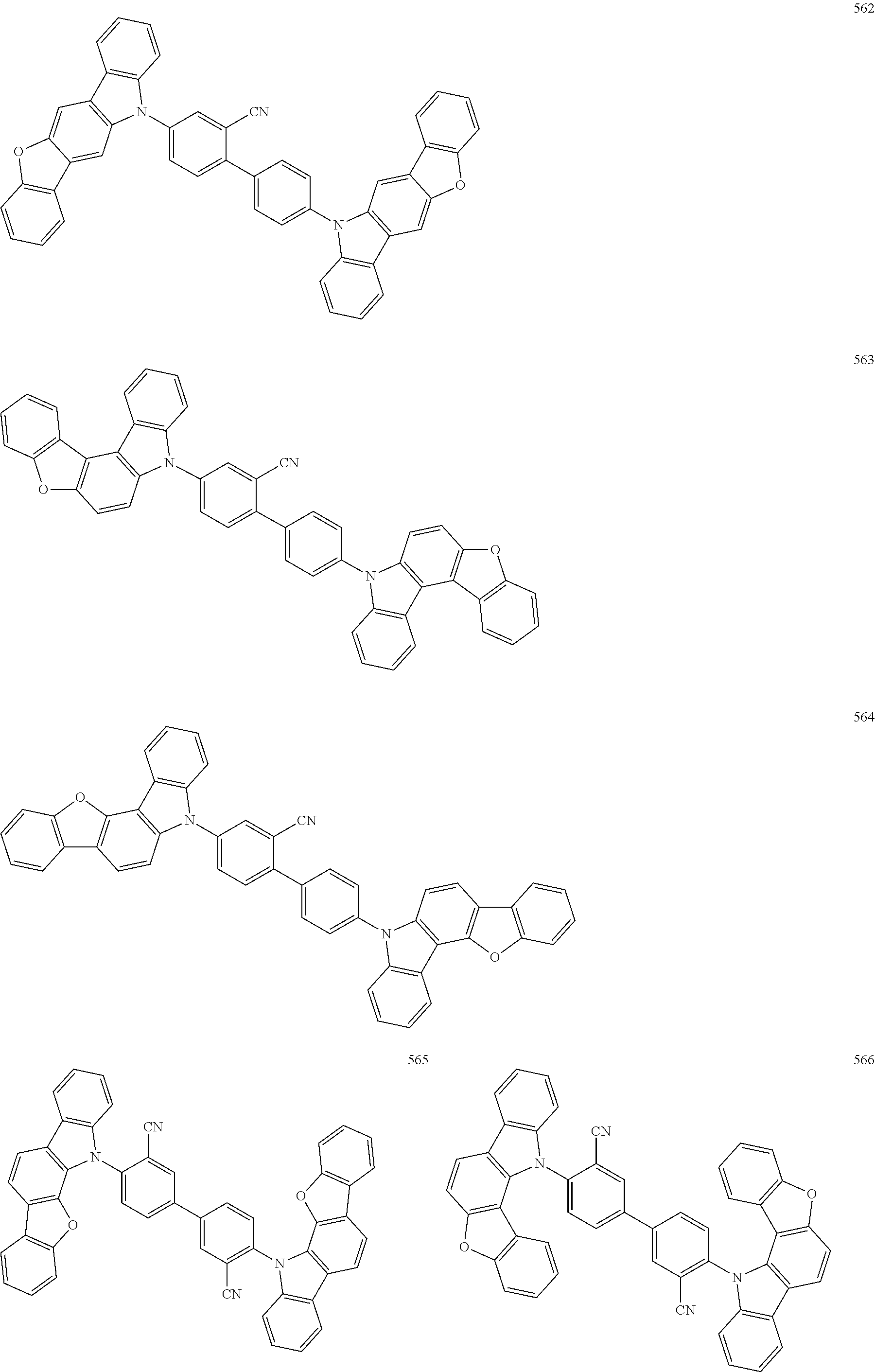 [SMILES: N#Cc1cc(-n2c3ccccc3c3cc4oc5ccccc5c4cc32)ccc1-c1ccc(-n2c3ccccc3c3cc4oc5ccccc5c4cc32)cc1 ; N#Cc1cc(-n2c3ccccc3c3c4c(ccc32)oc2ccccc24)ccc1-c1ccc(-n2c3ccccc3c3c4c(ccc32)oc2ccccc24)cc1 ; N#Cc1cc(-n2c3ccccc3c3c4oc5ccccc5c4ccc32)ccc1-c1ccc(-n2c3ccccc3c3c4oc5ccccc5c4ccc32)cc1 ; N#Cc1cc(-c2ccc(-n3c4ccccc4c4ccc5c6ccccc6oc5c43)c(C#N)c2)ccc1-n1c2ccccc2c2ccc3c4ccccc4oc3c21 ; N#Cc1cc(-c2ccc(-n3c4ccccc4c4ccc5oc6ccccc6c5c43)c(C#N)c2)ccc1-n1c2ccccc2c2ccc3oc4ccccc4c3c21]

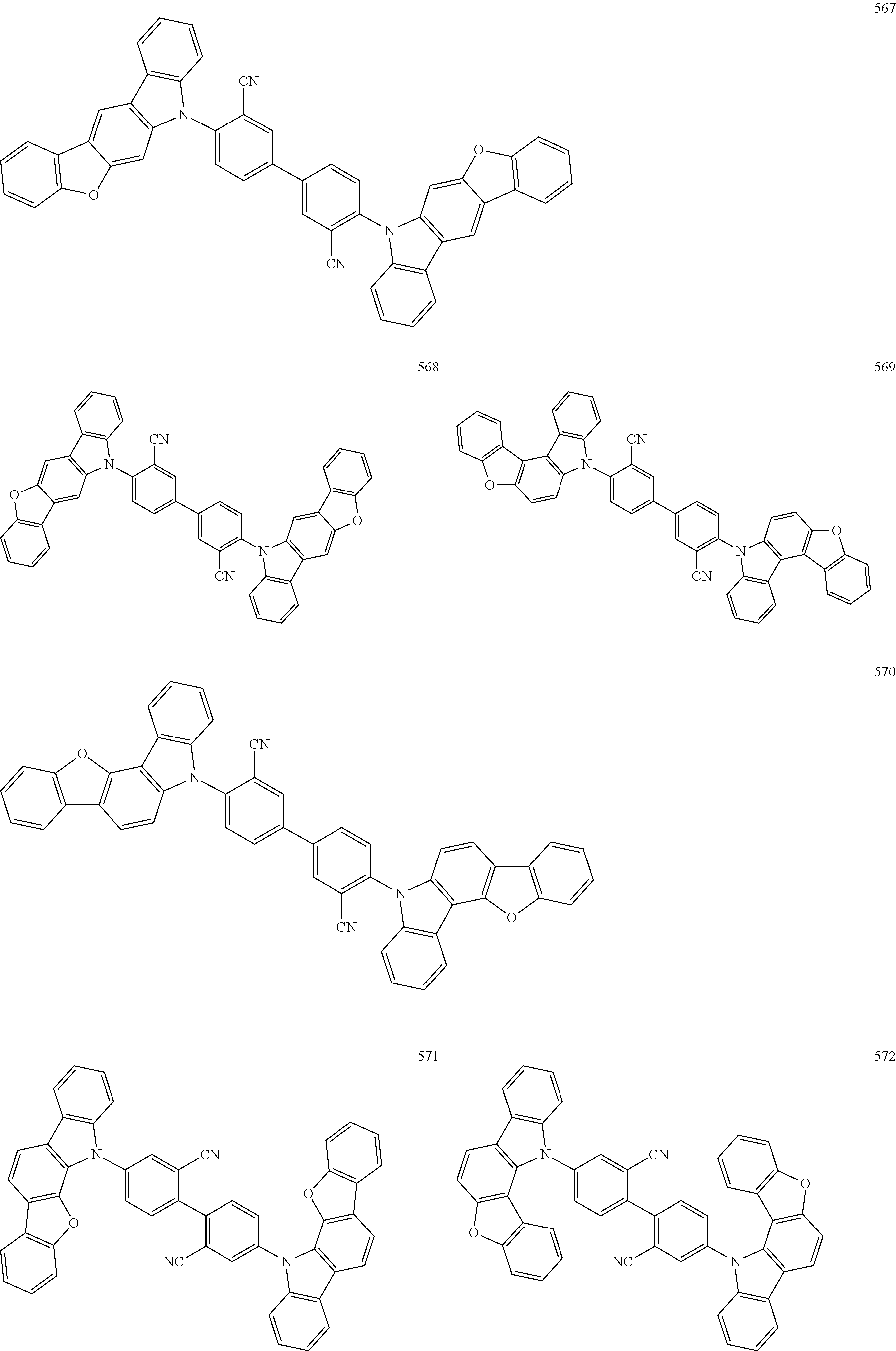

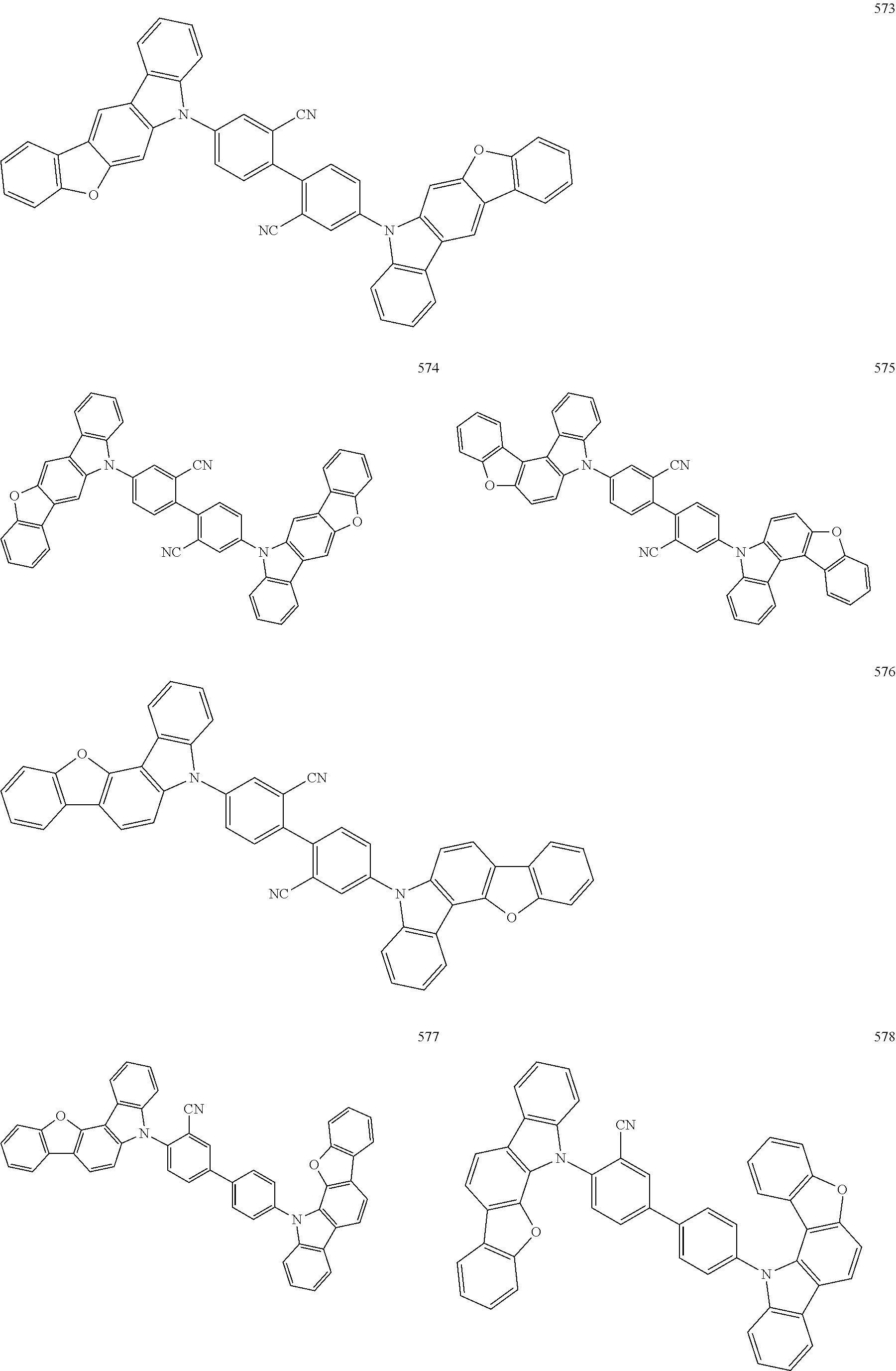

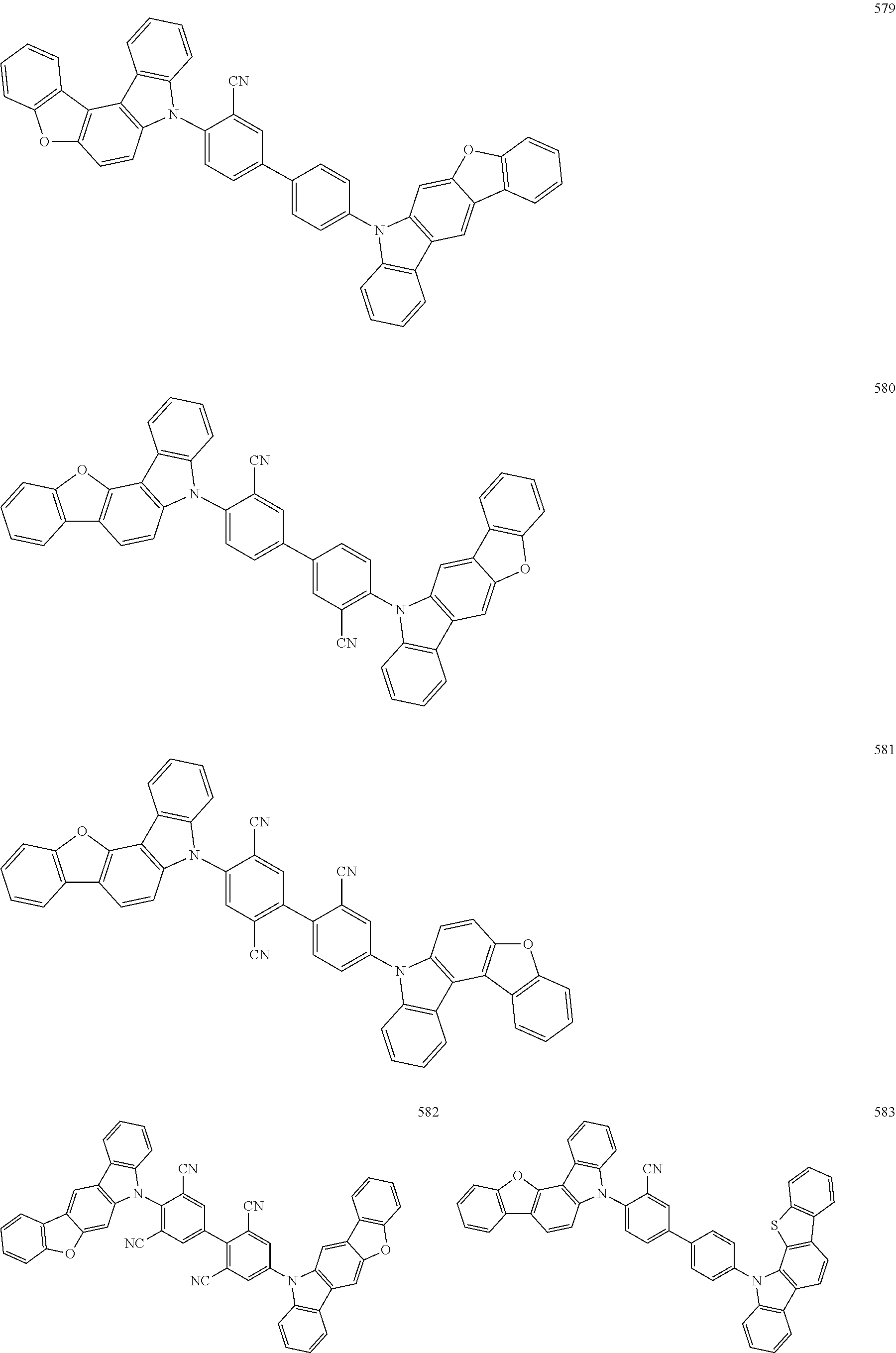 [SMILES: N#Cc1cc(-c2ccc(-n3c4ccccc4c4cc5c(cc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)c(C#N)c2)ccc1-n1c2ccccc2c2cc3oc4ccccc4c3cc21 ; N#Cc1cc(-n2c3ccccc3c3c4c(ccc32)oc2ccccc24)ccc1-c1cc(C#N)c(-n2c3ccccc3c3c4oc5ccccc5c4ccc32)cc1C#N ; N#Cc1cc(-n2c3ccccc3c3cc4oc5ccccc5c4cc32)cc(C#N)c1-c1cc(C#N)c(-n2c3ccccc3c3cc4c(cc32)oc2ccccc24)c(C#N)c1 ; N#Cc1cc(-c2ccc(-n3c4ccccc4c4ccc5c6ccccc6sc5c43)cc2)ccc1-n1c2ccccc2c2c3oc4ccccc4c3ccc21]

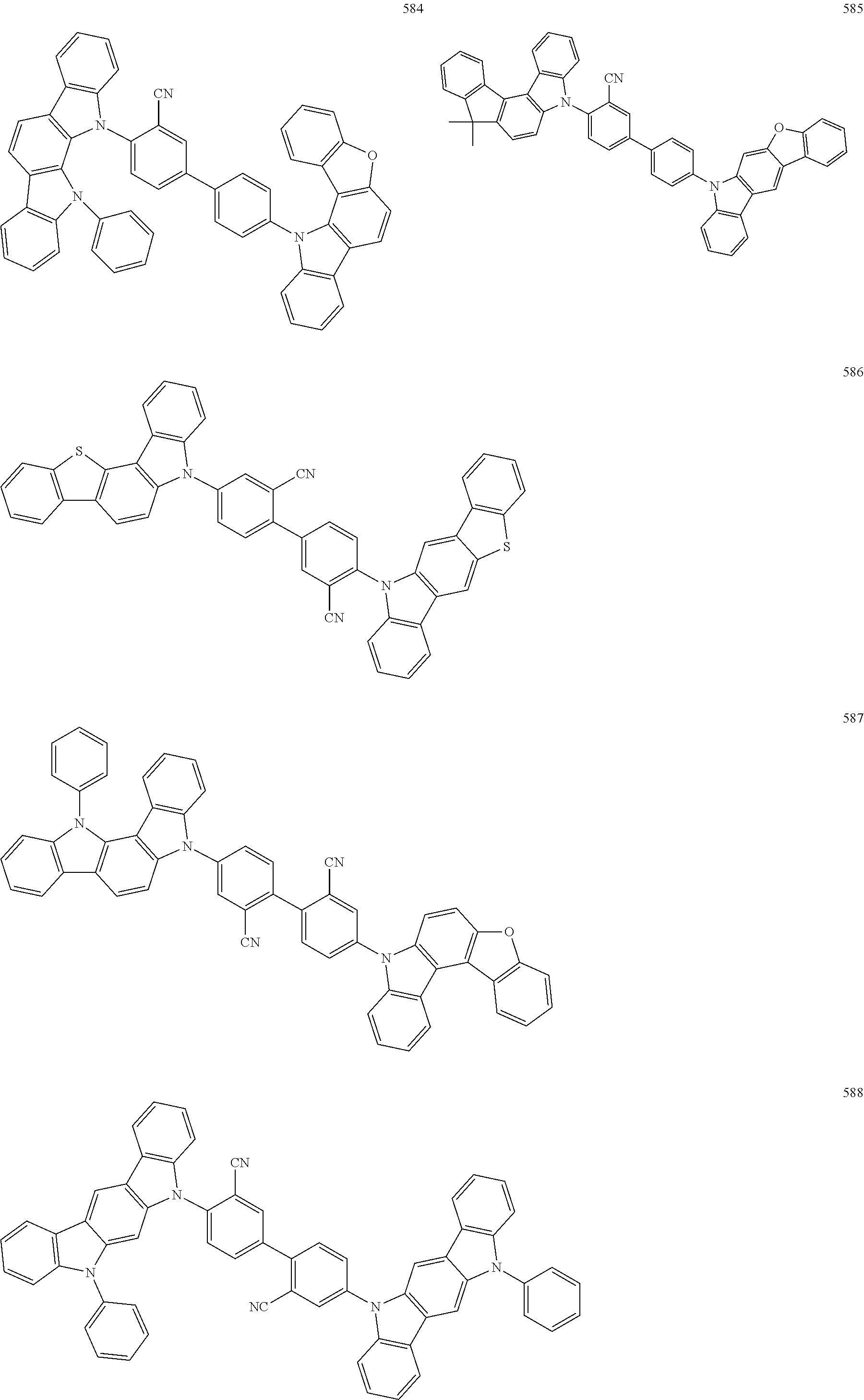

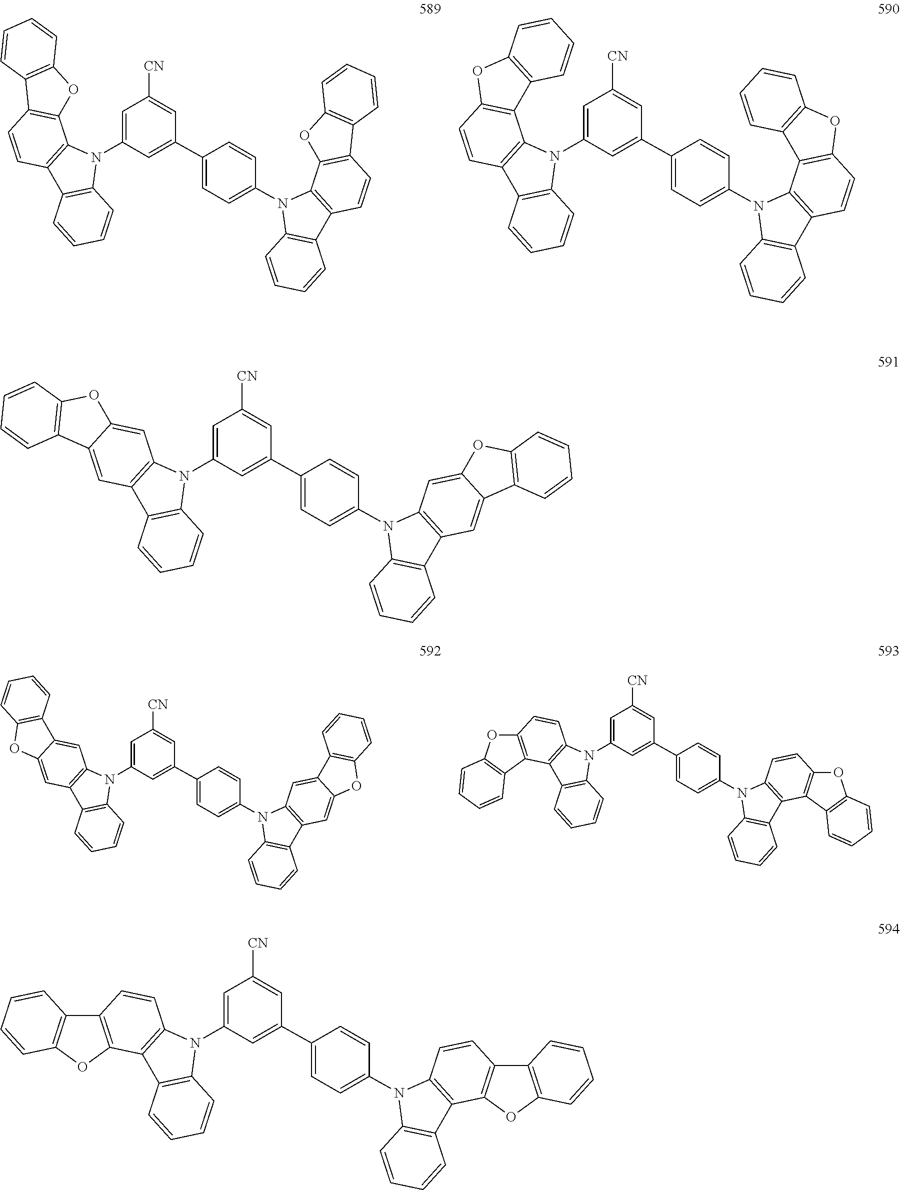

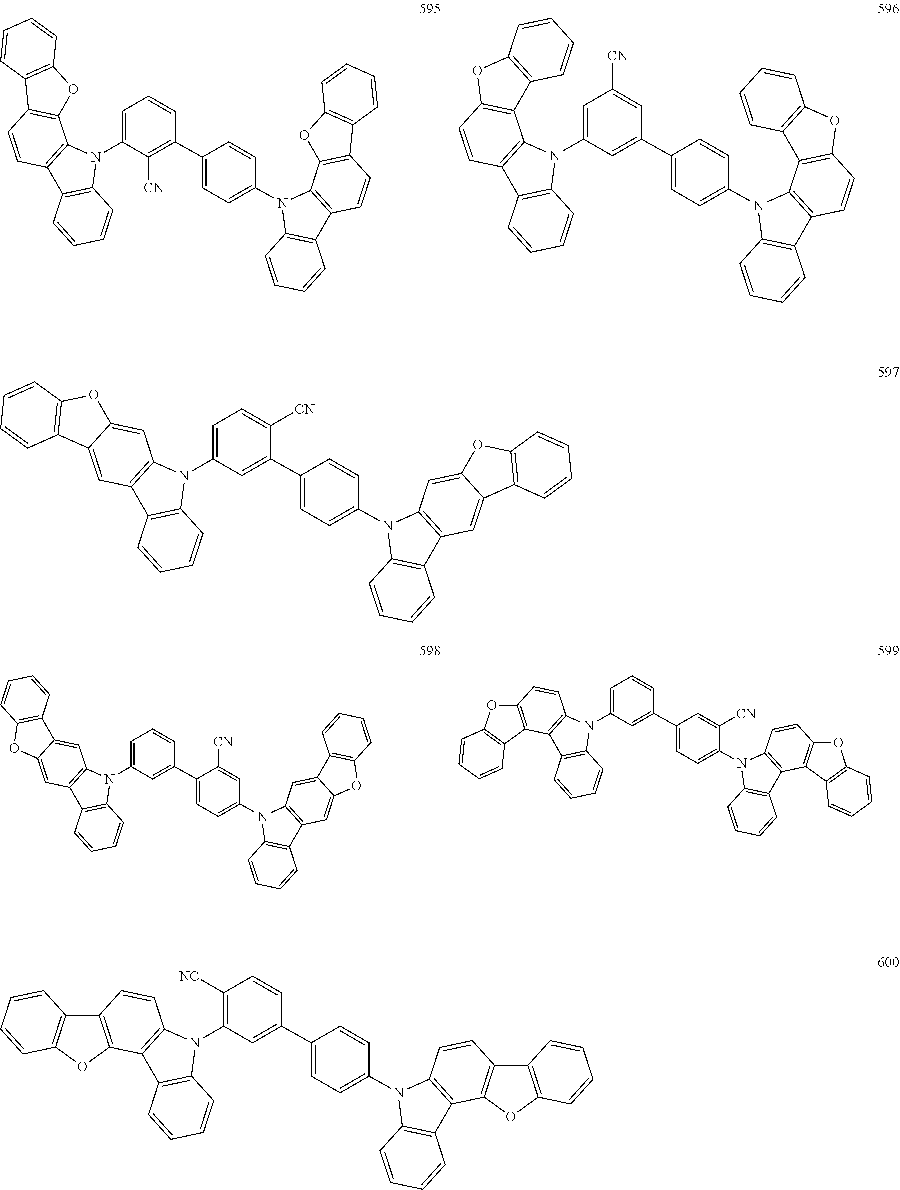

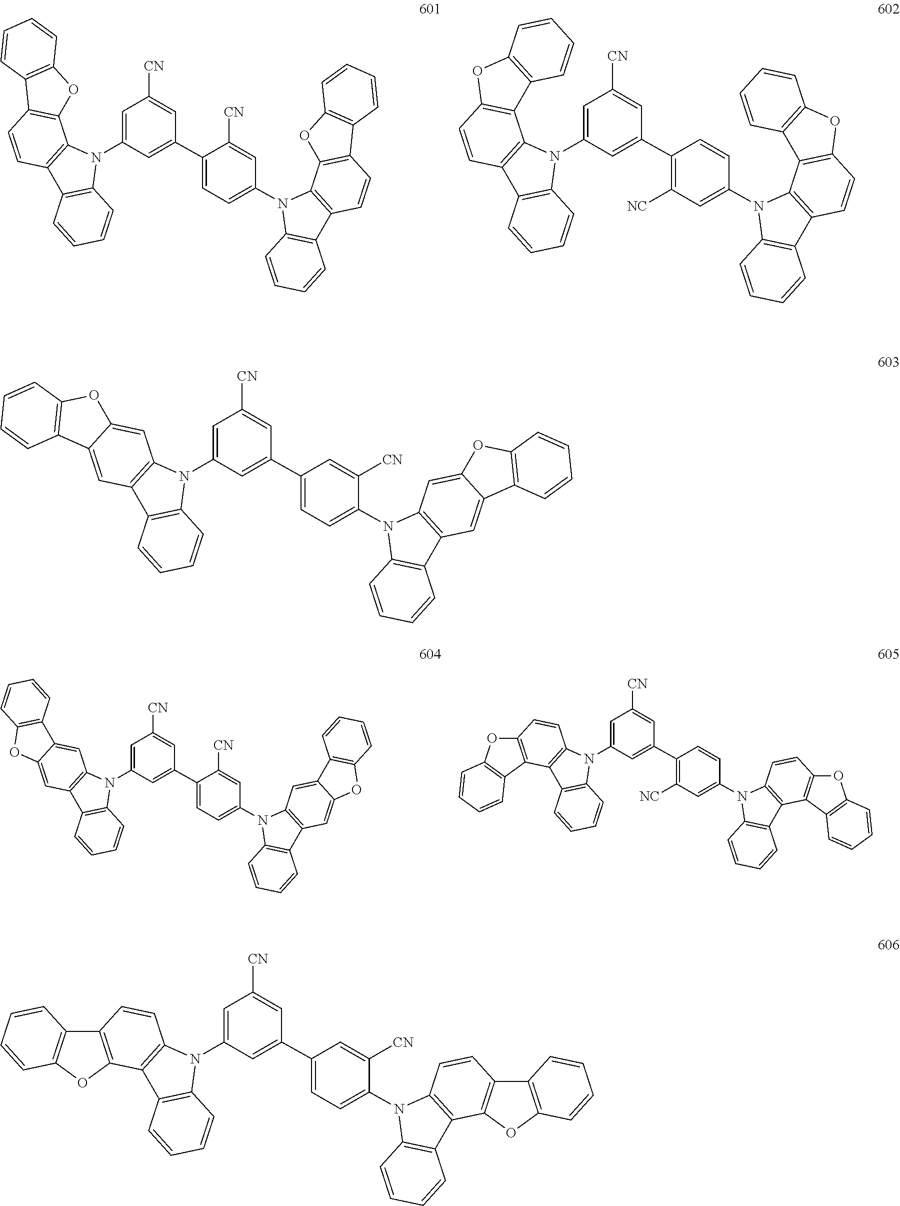

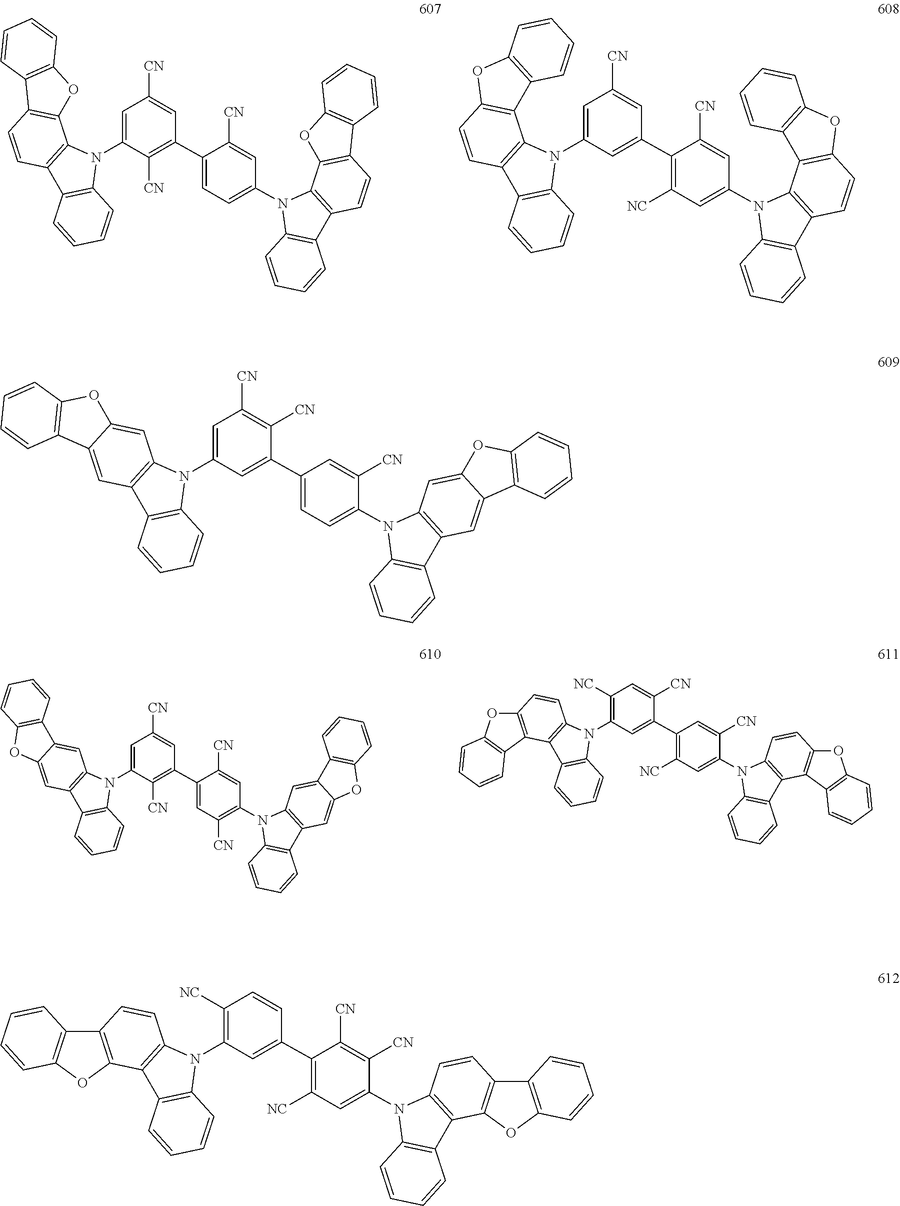

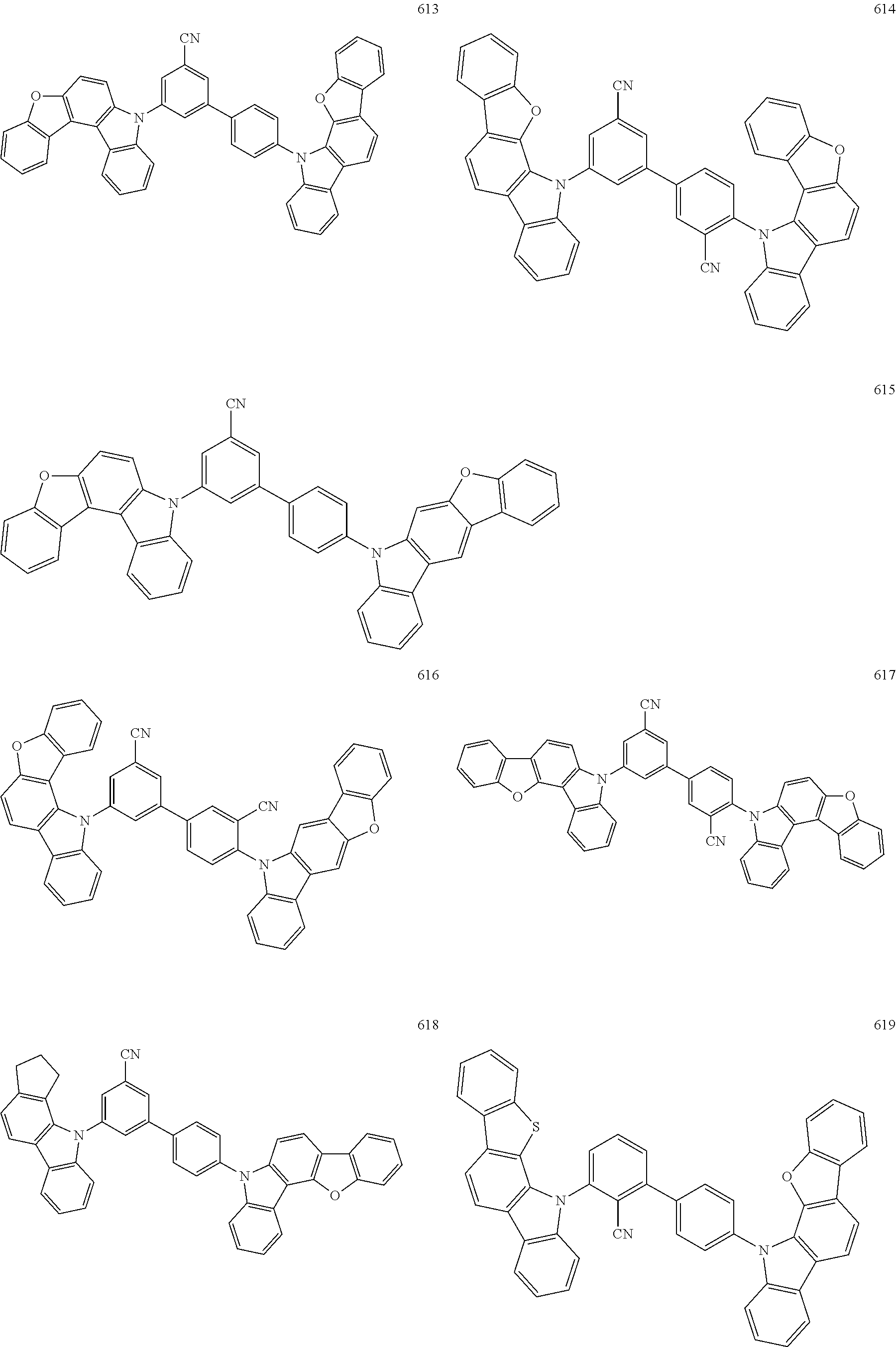

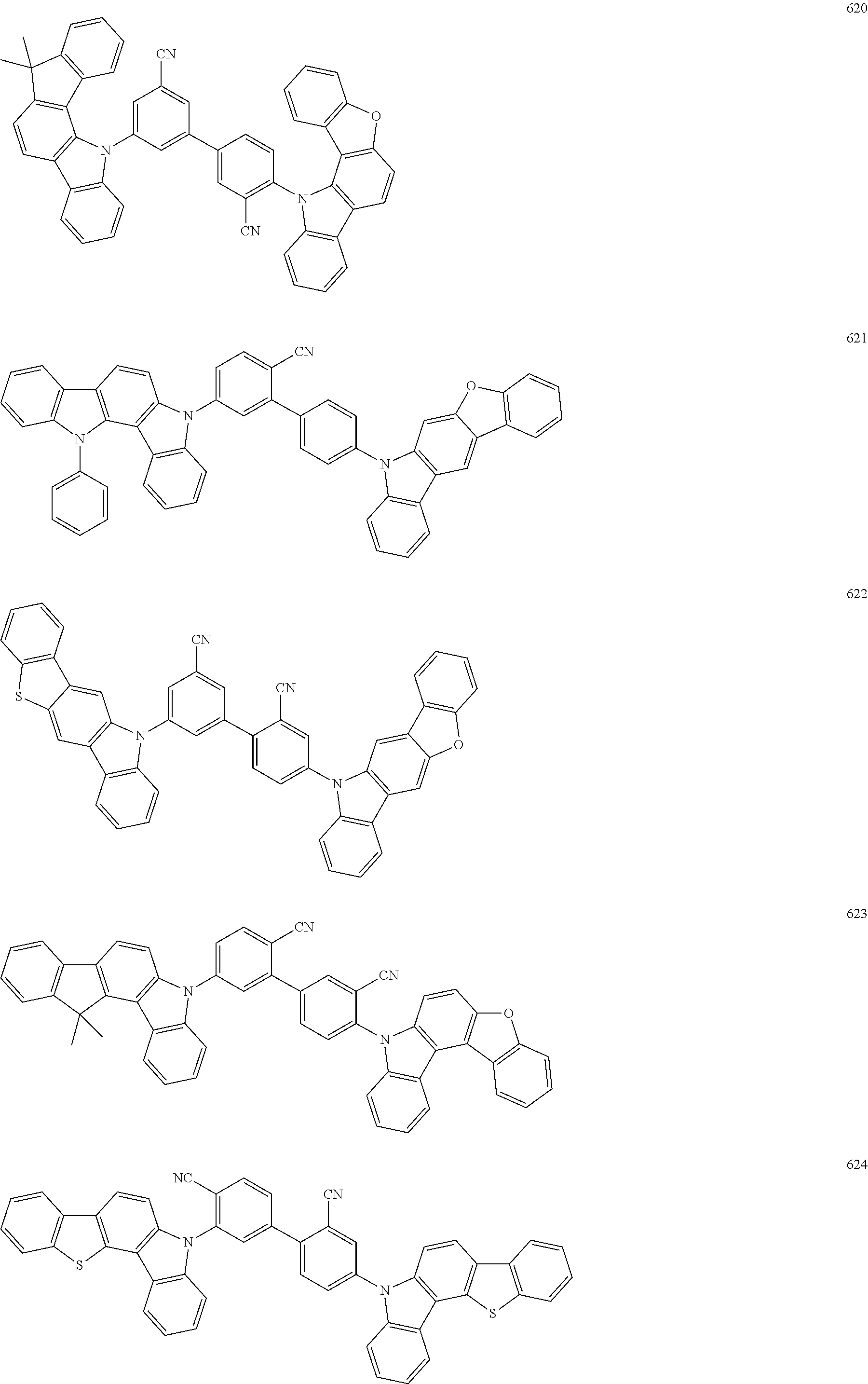 [SMILES: CC1(C)c2ccccc2-c2c1ccc1c3ccccc3n(-c3cc(C#N)cc(-c4ccc(-n5c6ccccc6c6ccc7oc8ccccc8c7c65)c(C#N)c4)c3)c21 ; N#Cc1ccc(-n2c3ccccc3c3c2ccc2c4ccccc4n(-c4ccccc4)c23)cc1-c1ccc(-n2c3ccccc3c3cc4c(cc32)oc2ccccc24)cc1 ; N#Cc1cc(-c2ccc(-n3c4ccccc4c4cc5oc6ccccc6c5cc43)cc2C#N)cc(-n2c3ccccc3c3cc4sc5ccccc5c4cc32)c1 ; CC1(C)c2ccccc2-c2ccc3c(c21)c1ccccc1n3-c1ccc(C#N)c(-c2ccc(-n3c4ccccc4c4c5c(ccc43)oc3ccccc35)c(C#N)c2)c1 ; N#Cc1cc(-n2c3ccccc3c3c4sc5ccccc5c4ccc32)ccc1-c1ccc(C#N)c(-n2c3ccccc3c3c4sc5ccccc5c4ccc32)c1]

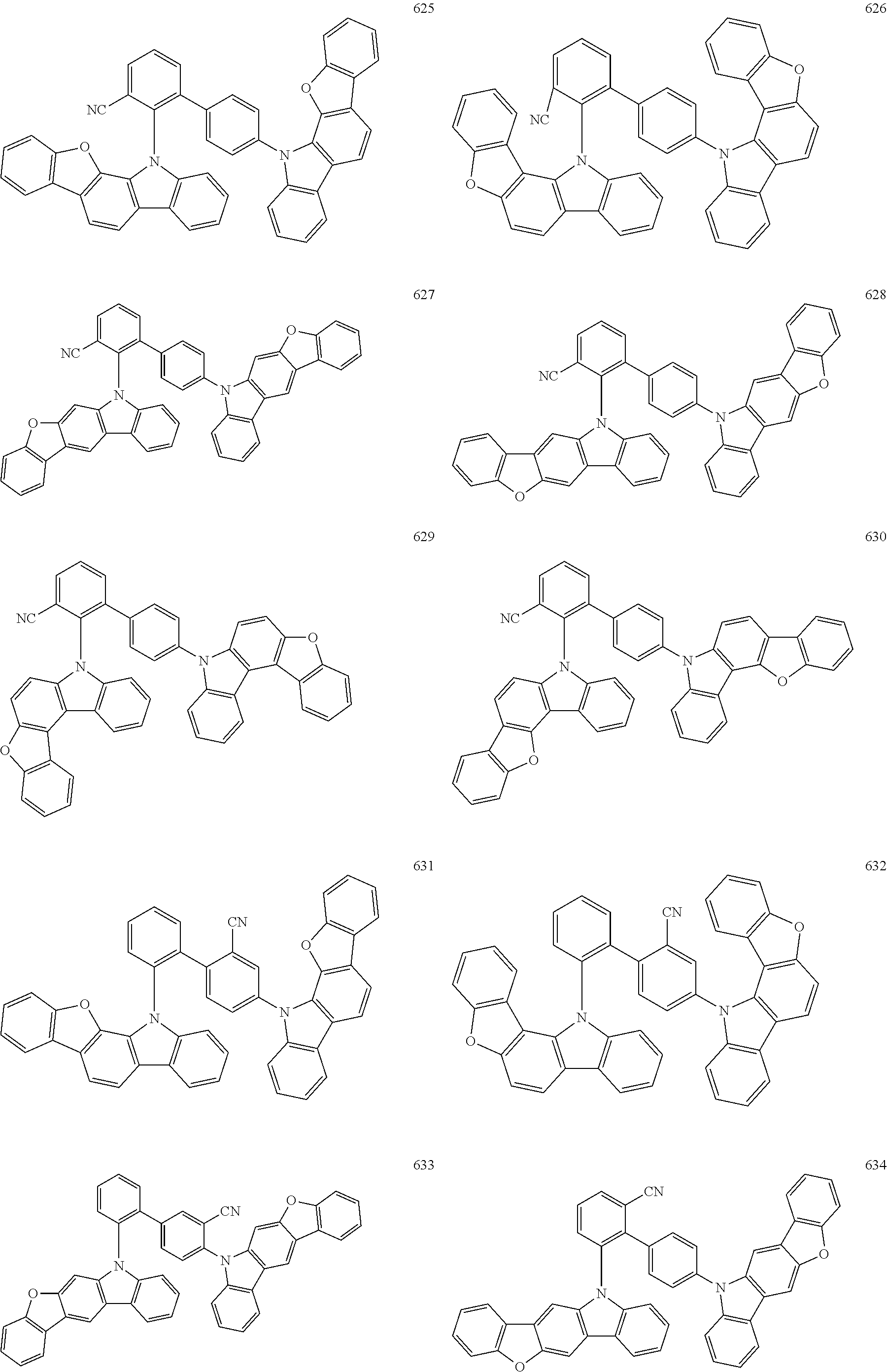

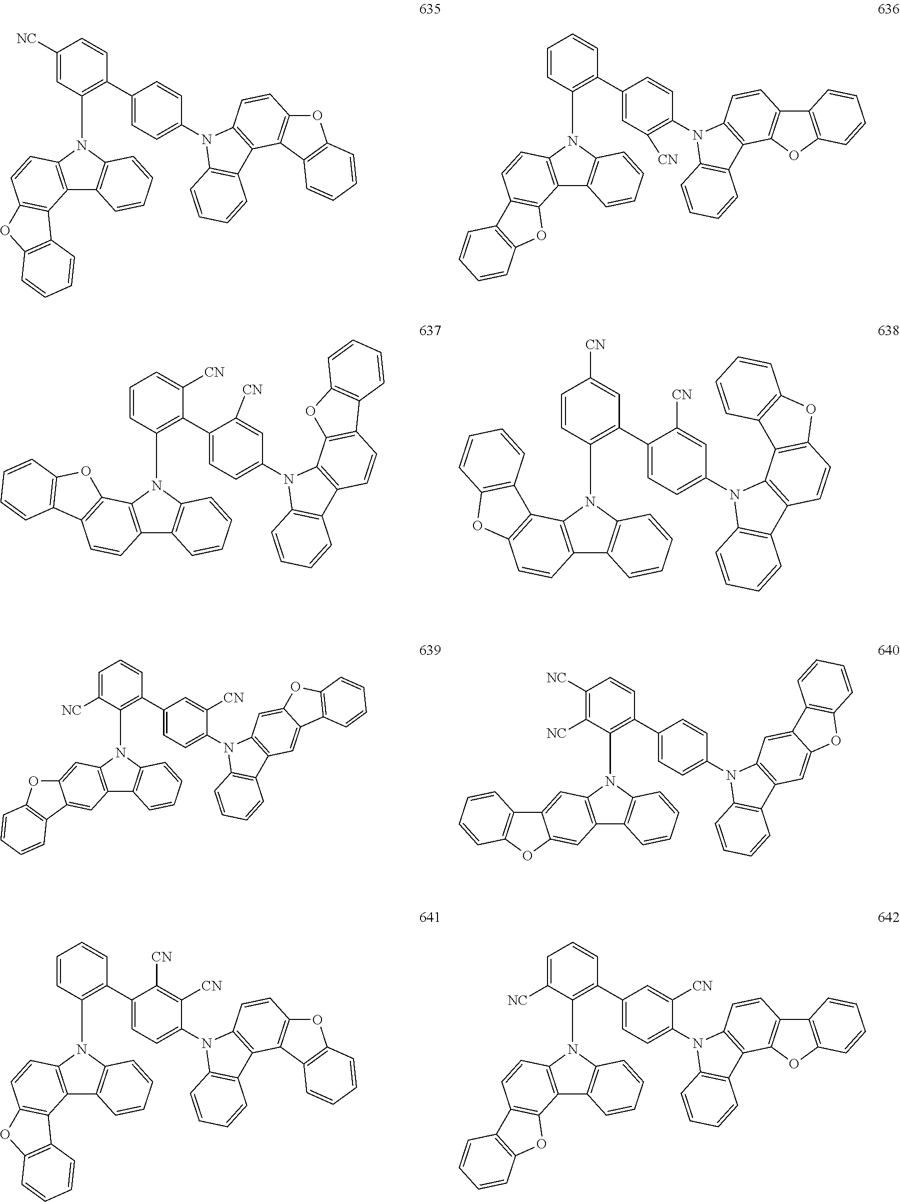

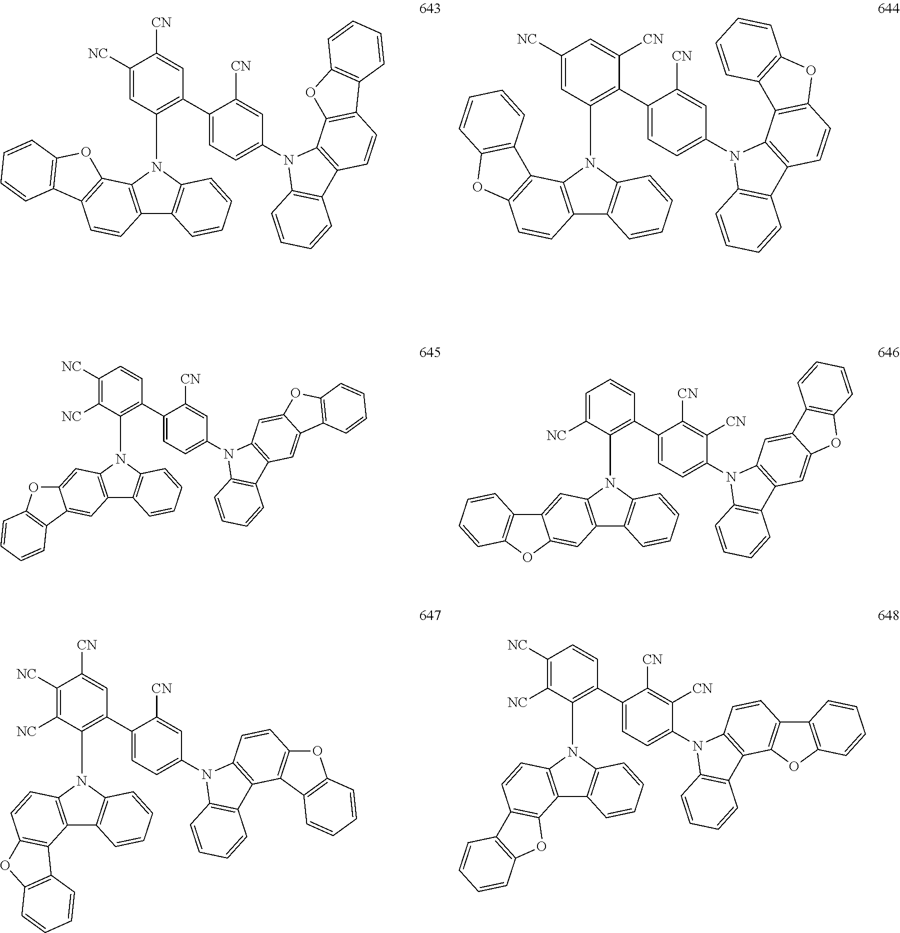

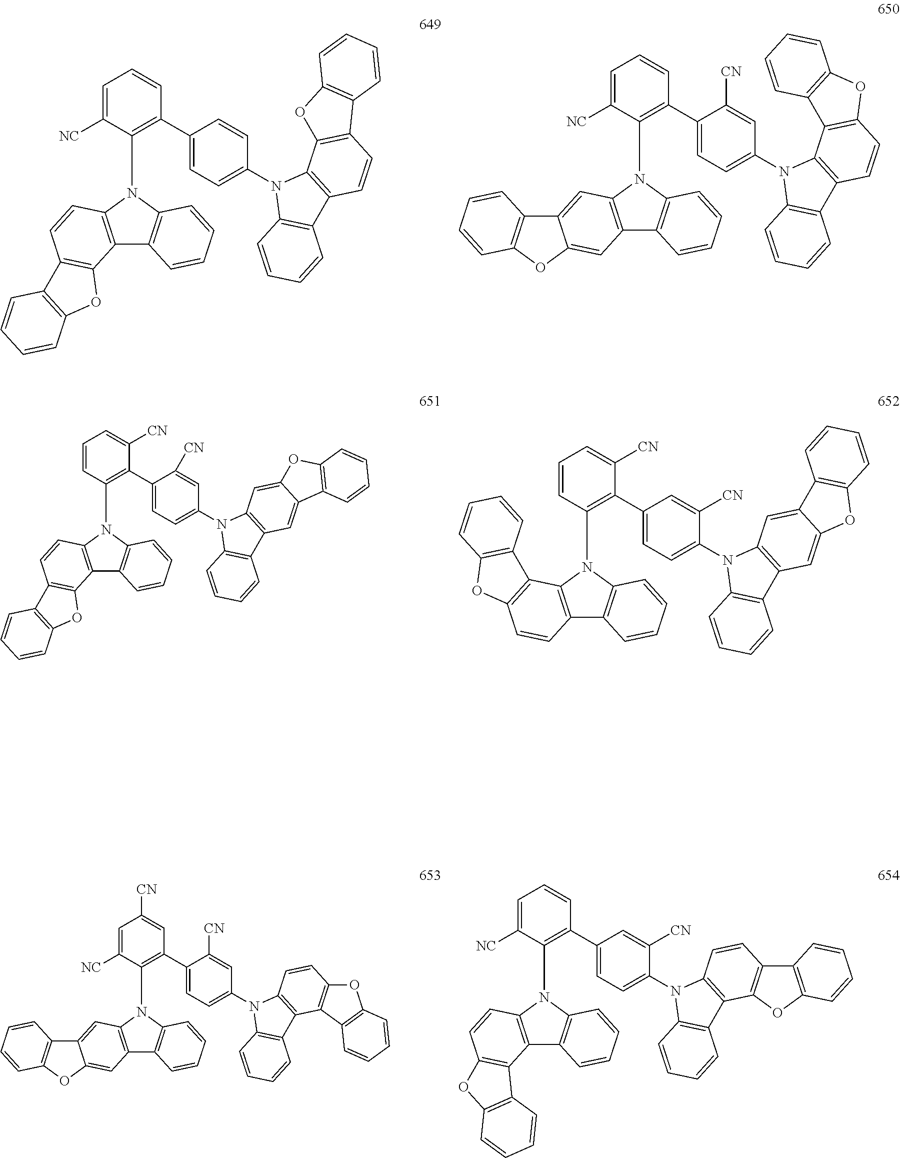

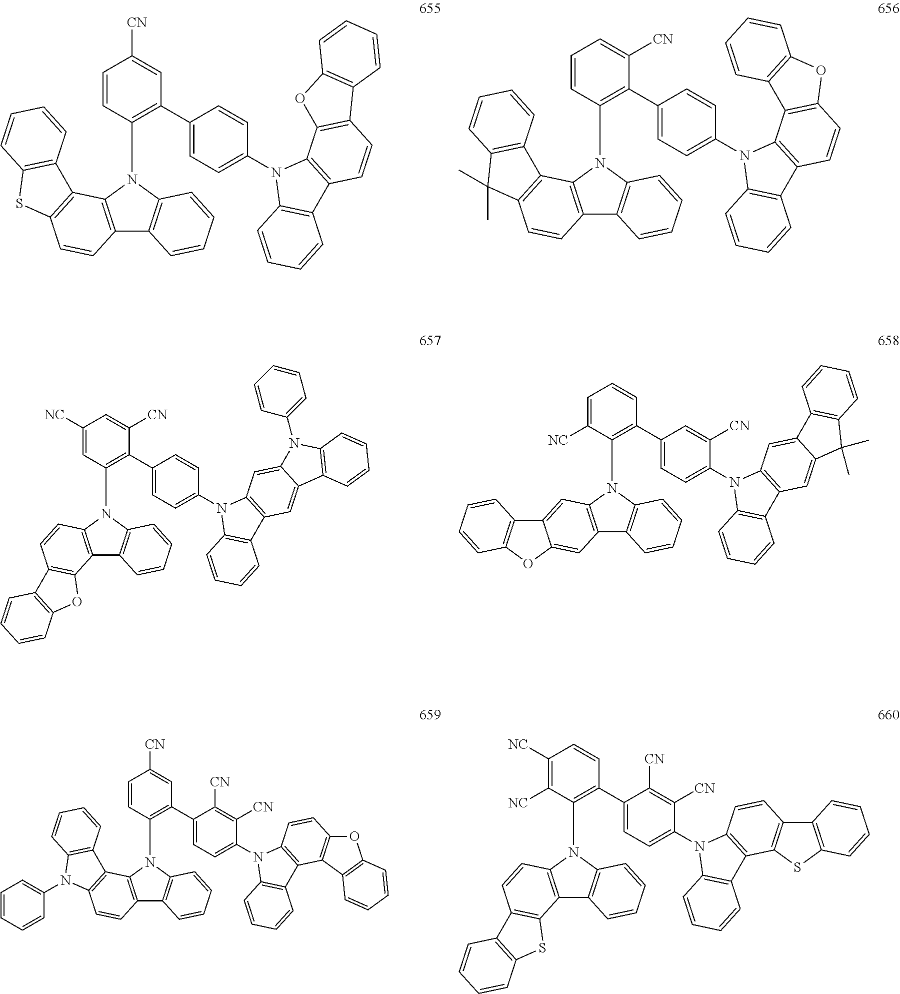

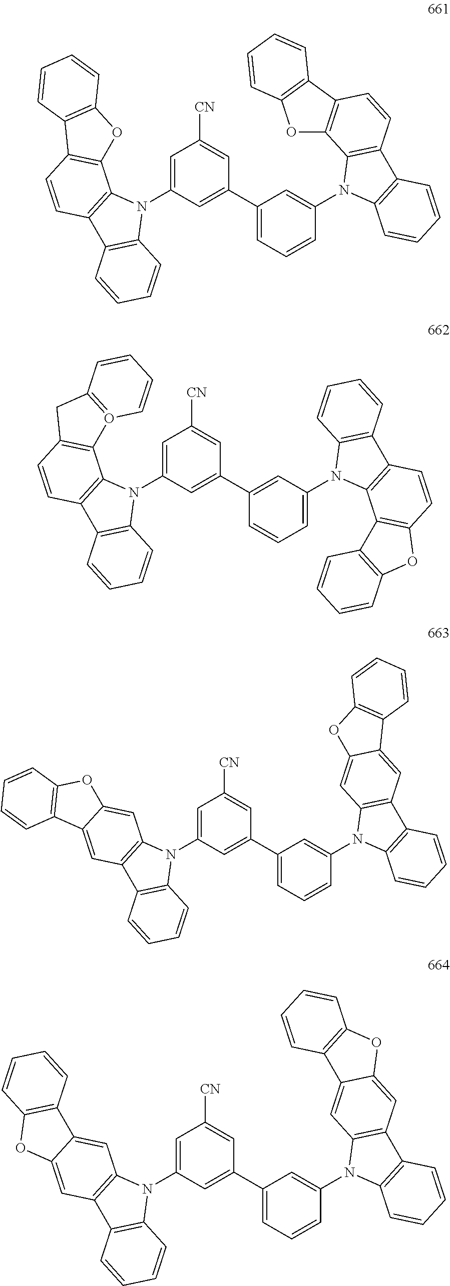

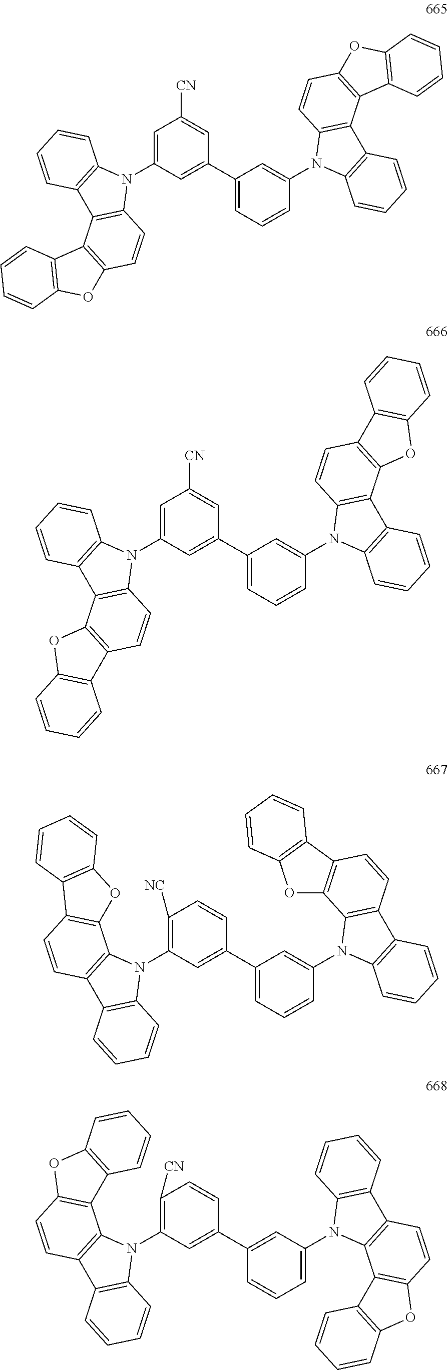 [SMILES: N#Cc1cc(-c2cccc(-n3c4ccccc4c4c5c(ccc43)oc3ccccc35)c2)cc(-n2c3ccccc3c3c4c(ccc32)oc2ccccc24)c1 ; N#Cc1cc(-c2cccc(-n3c4ccccc4c4c5oc6ccccc6c5ccc43)c2)cc(-n2c3ccccc3c3c4oc5ccccc5c4ccc32)c1 ; N#Cc1ccc(-c2cccc(-n3c4ccccc4c4ccc5c6ccccc6oc5c43)c2)cc1-n1c2ccccc2c2ccc3c4ccccc4oc3c21 ; N#Cc1ccc(-c2cccc(-n3c4ccccc4c4ccc5oc6ccccc6c5c43)c2)cc1-n1c2ccccc2c2ccc3oc4ccccc4c3c21]

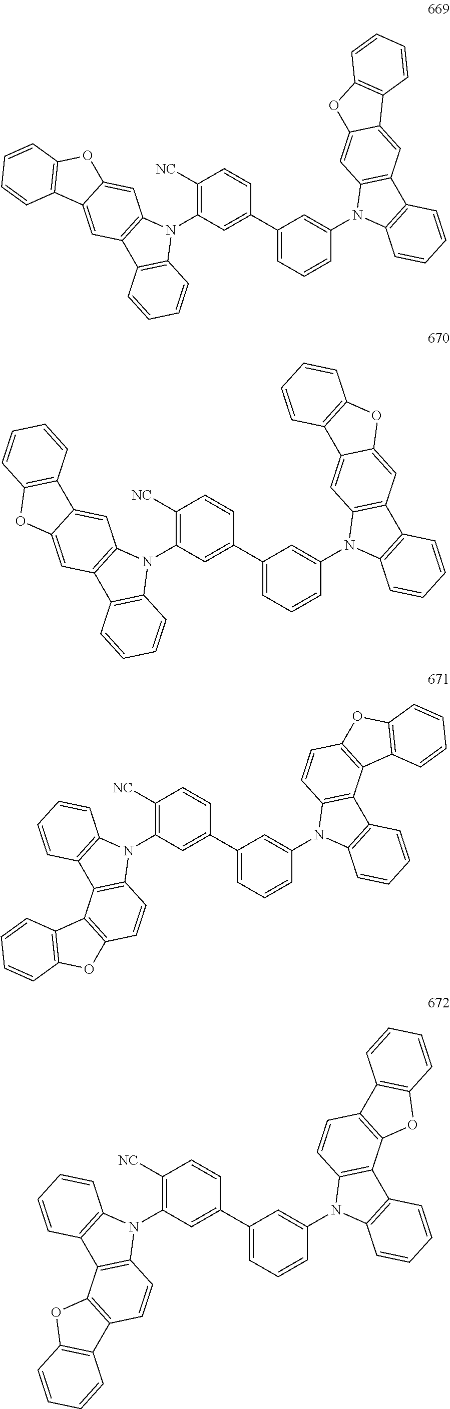 [SMILES: N#Cc1ccc(-c2cccc(-n3c4ccccc4c4cc5c(cc43)oc3ccccc35)c2)cc1-n1c2ccccc2c2cc3c(cc21)oc1ccccc13 ; N#Cc1ccc(-c2cccc(-n3c4ccccc4c4cc5oc6ccccc6c5cc43)c2)cc1-n1c2ccccc2c2cc3oc4ccccc4c3cc21 ; N#Cc1ccc(-c2cccc(-n3c4ccccc4c4c5c(ccc43)oc3ccccc35)c2)cc1-n1c2ccccc2c2c3c(ccc21)oc1ccccc13 ; N#Cc1ccc(-c2cccc(-n3c4ccccc4c4c5oc6ccccc6c5ccc43)c2)cc1-n1c2ccccc2c2c3oc4ccccc4c3ccc21]

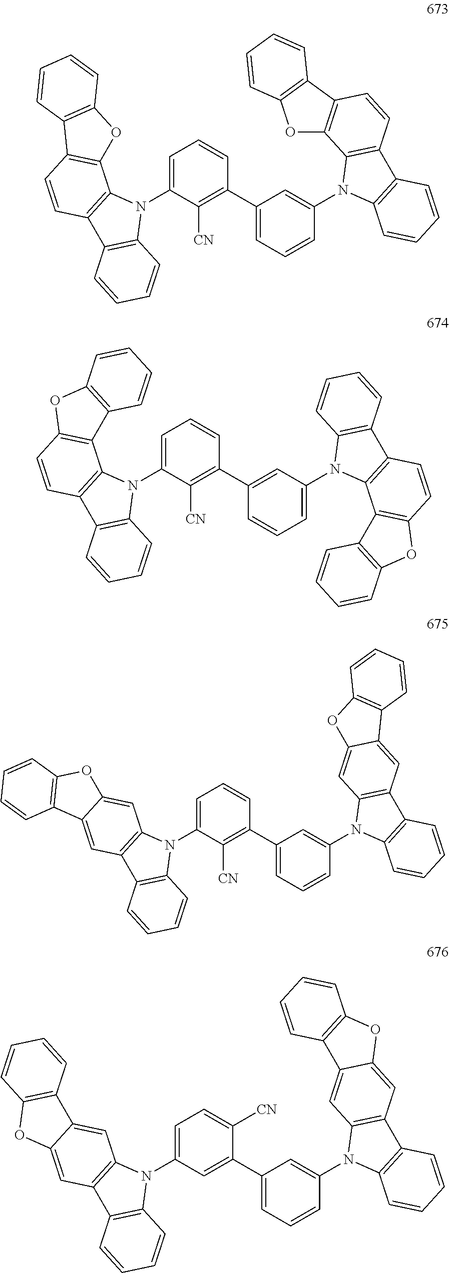

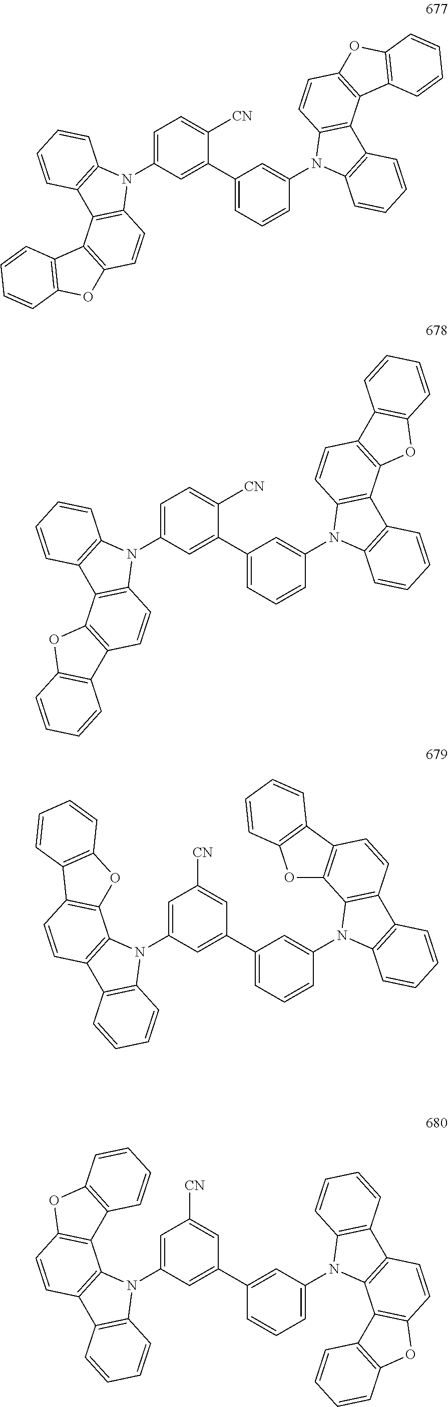 [SMILES: N#Cc1ccc(-n2c3ccccc3c3c4c(ccc32)oc2ccccc24)cc1-c1cccc(-n2c3ccccc3c3c4c(ccc32)oc2ccccc24)c1 ; N#Cc1ccc(-n2c3ccccc3c3c4oc5ccccc5c4ccc32)cc1-c1cccc(-n2c3ccccc3c3c4oc5ccccc5c4ccc32)c1 ; N#Cc1cc(-c2cccc(-n3c4ccccc4c4ccc5c6ccccc6oc5c43)c2)cc(-n2c3ccccc3c3ccc4c5ccccc5oc4c32)c1 ; N#Cc1cc(-c2cccc(-n3c4ccccc4c4ccc5oc6ccccc6c5c43)c2)cc(-n2c3ccccc3c3ccc4oc5ccccc5c4c32)c1]

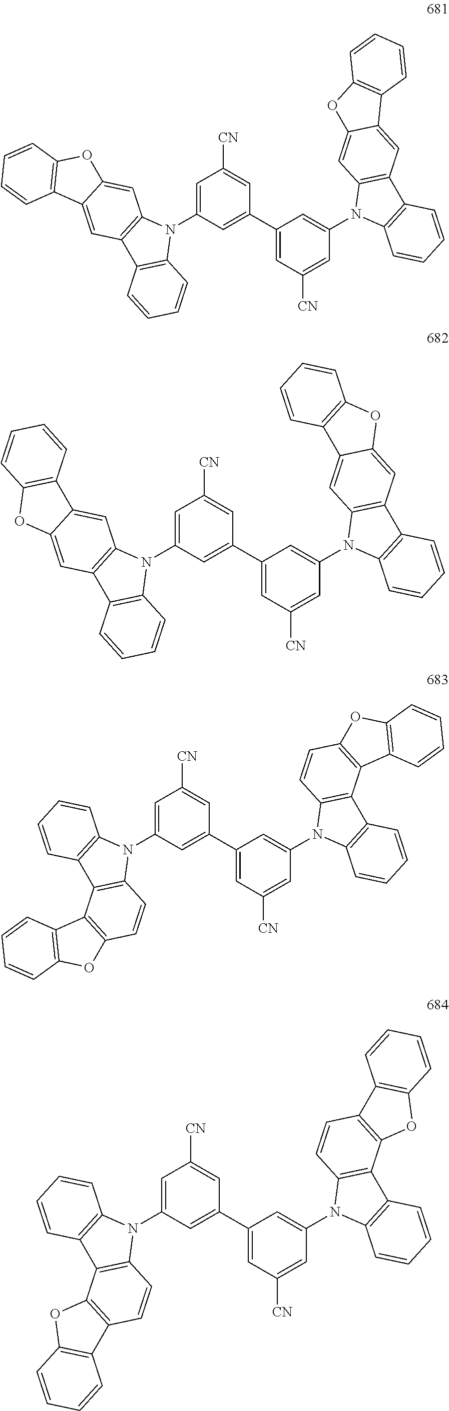 [SMILES: N#Cc1cc(-c2cc(C#N)cc(-n3c4ccccc4c4cc5c(cc43)oc3ccccc35)c2)cc(-n2c3ccccc3c3cc4c(cc32)oc2ccccc24)c1 ; N#Cc1cc(-c2cc(C#N)cc(-n3c4ccccc4c4cc5oc6ccccc6c5cc43)c2)cc(-n2c3ccccc3c3cc4oc5ccccc5c4cc32)c1 ; N#Cc1cc(-c2cc(C#N)cc(-n3c4ccccc4c4c5c(ccc43)oc3ccccc35)c2)cc(-n2c3ccccc3c3c4c(ccc32)oc2ccccc24)c1 ; N#Cc1cc(-c2cc(C#N)cc(-n3c4ccccc4c4c5oc6ccccc6c5ccc43)c2)cc(-n2c3ccccc3c3c4oc5ccccc5c4ccc32)c1]

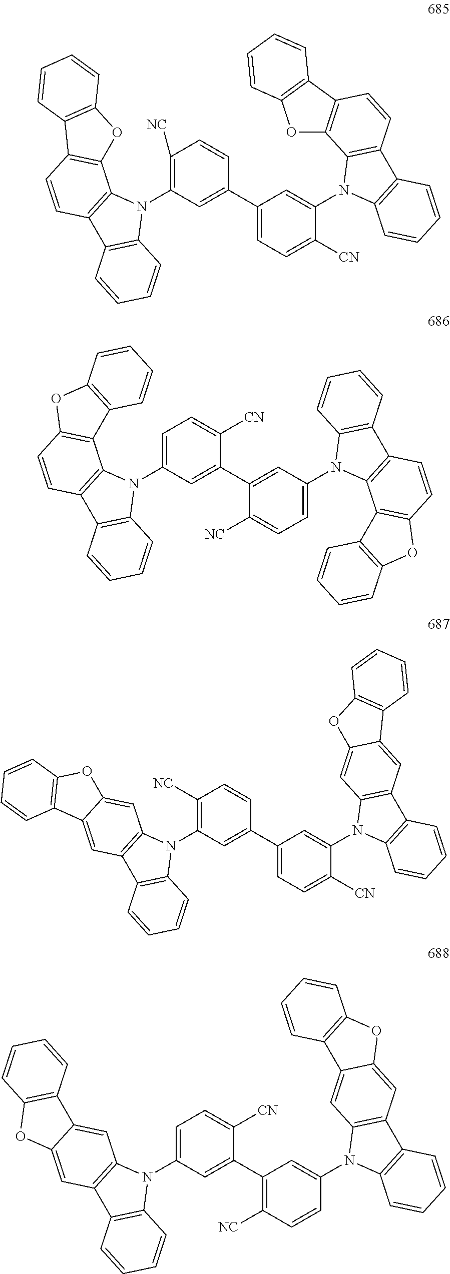

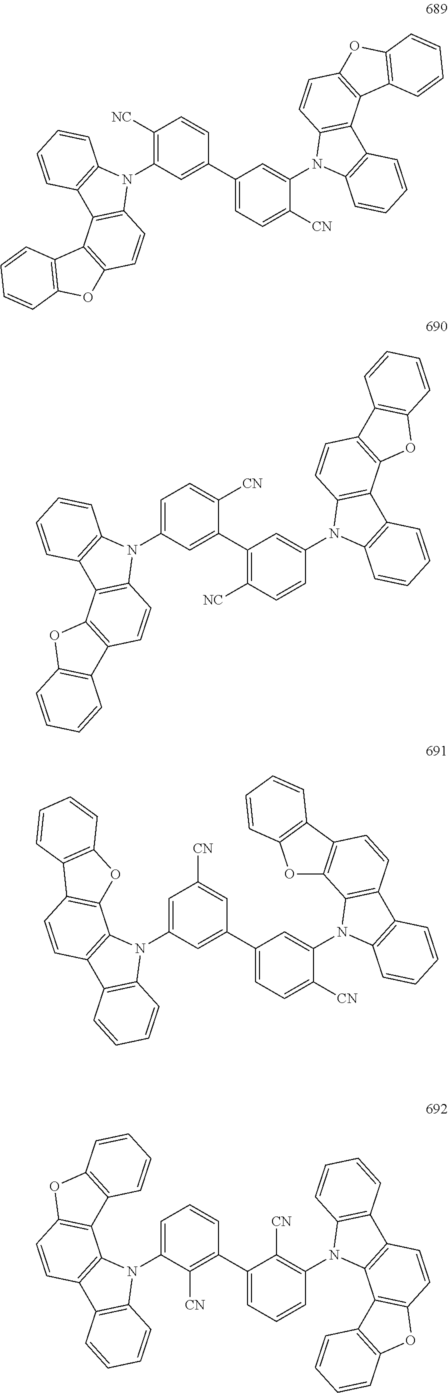 [SMILES: N#Cc1ccc(-c2ccc(C#N)c(-n3c4ccccc4c4c5c(ccc43)oc3ccccc35)c2)cc1-n1c2ccccc2c2c3c(ccc21)oc1ccccc13 ; N#Cc1ccc(-n2c3ccccc3c3c4oc5ccccc5c4ccc32)cc1-c1cc(-n2c3ccccc3c3c4oc5ccccc5c4ccc32)ccc1C#N ; N#Cc1cc(-c2ccc(C#N)c(-n3c4ccccc4c4ccc5c6ccccc6oc5c43)c2)cc(-n2c3ccccc3c3ccc4c5ccccc5oc4c32)c1 ; N#Cc1c(-c2cccc(-n3c4ccccc4c4ccc5oc6ccccc6c5c43)c2C#N)cccc1-n1c2ccccc2c2ccc3oc4ccccc4c3c21]

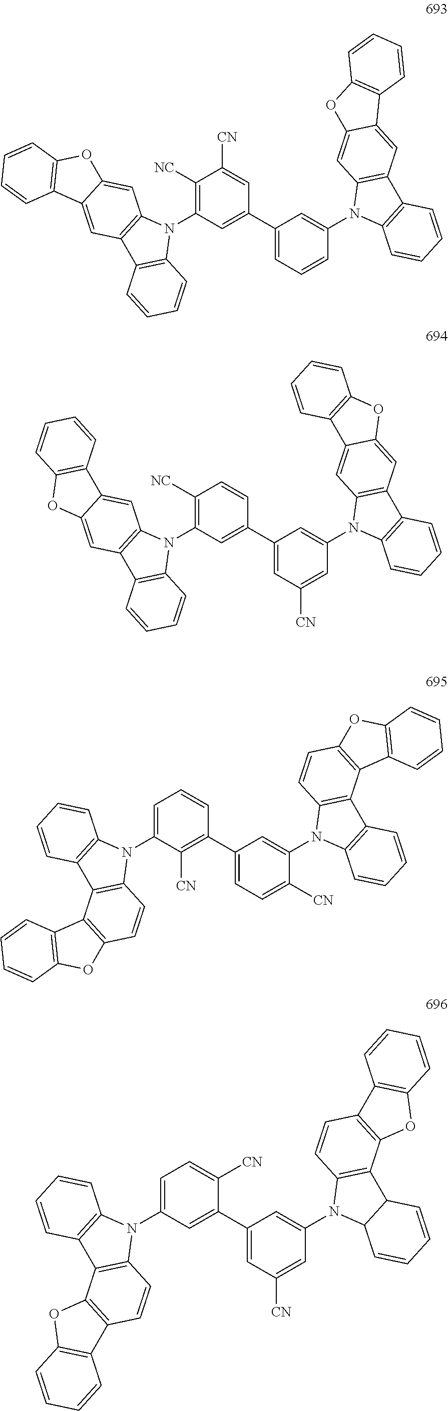 [SMILES: N#Cc1cc(-c2cccc(-n3c4ccccc4c4cc5c(cc43)oc3ccccc35)c2)cc(-n2c3ccccc3c3cc4c(cc32)oc2ccccc24)c1C#N ; N#Cc1cc(-c2ccc(C#N)c(-n3c4ccccc4c4cc5oc6ccccc6c5cc43)c2)cc(-n2c3ccccc3c3cc4oc5ccccc5c4cc32)c1 ; N#Cc1ccc(-c2cccc(-n3c4ccccc4c4c5c(ccc43)oc3ccccc35)c2C#N)cc1-n1c2ccccc2c2c3c(ccc21)oc1ccccc13 ; N#Cc1cc(-c2cc(-n3c4ccccc4c4c5oc6ccccc6c5ccc43)ccc2C#N)cc(N2c3ccc4c(oc5ccccc54)c3C3C=CC=CC32)c1]

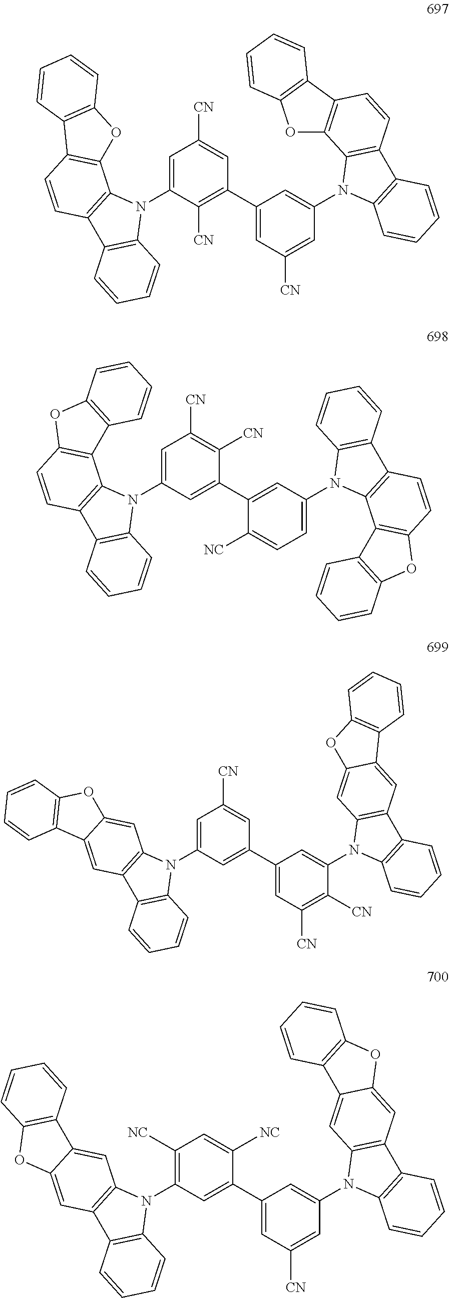

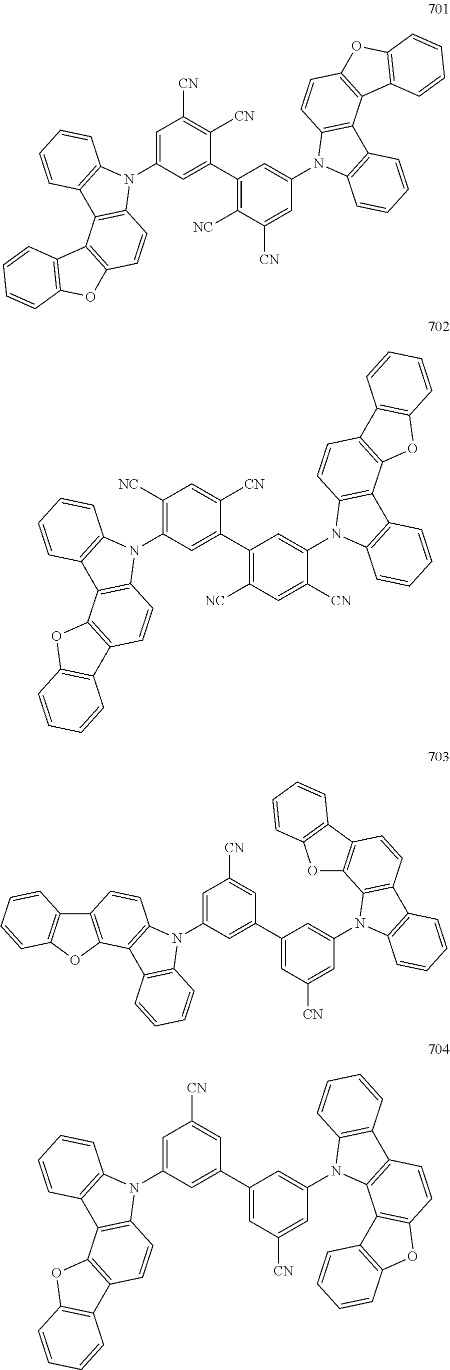 [SMILES: N#Cc1cc(-n2c3ccccc3c3c4c(ccc32)oc2ccccc24)cc(-c2cc(-n3c4ccccc4c4c5c(ccc43)oc3ccccc35)cc(C#N)c2C#N)c1C#N ; N#Cc1cc(C#N)c(-n2c3ccccc3c3c4oc5ccccc5c4ccc32)cc1-c1cc(-n2c3ccccc3c3c4oc5ccccc5c4ccc32)c(C#N)cc1C#N ; N#Cc1cc(-c2cc(C#N)cc(-n3c4ccccc4c4ccc5c6ccccc6oc5c43)c2)cc(-n2c3ccccc3c3c4oc5ccccc5c4ccc32)c1 ; N#Cc1cc(-c2cc(C#N)cc(-n3c4ccccc4c4ccc5oc6ccccc6c5c43)c2)cc(-n2c3ccccc3c3c4oc5ccccc5c4ccc32)c1]

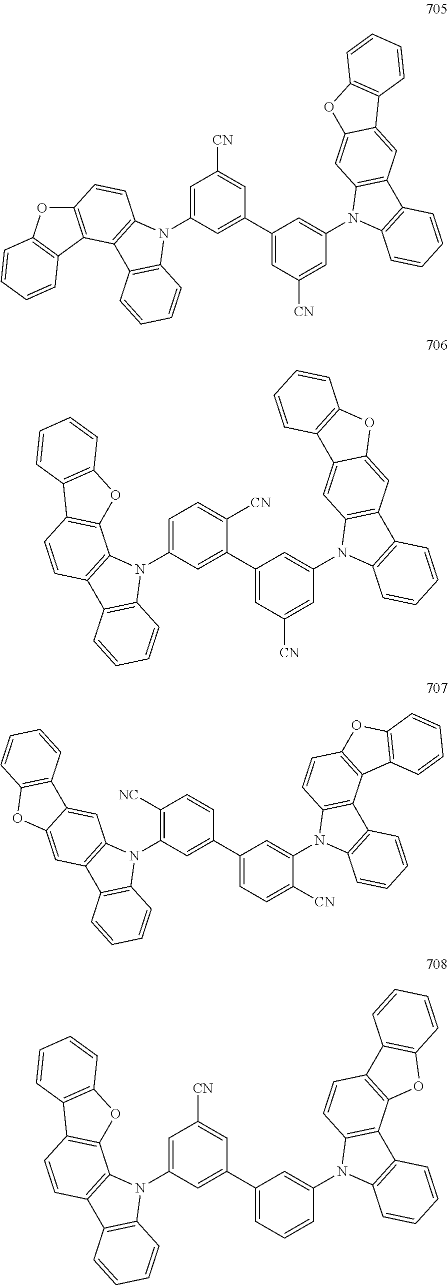 [SMILES: N#Cc1cc(-c2cc(C#N)cc(-n3c4ccccc4c4c5c(ccc43)oc3ccccc35)c2)cc(-n2c3ccccc3c3cc4c(cc32)oc2ccccc24)c1 ; N#Cc1cc(-c2cc(-n3c4ccccc4c4ccc5c6ccccc6oc5c43)ccc2C#N)cc(-n2c3ccccc3c3cc4oc5ccccc5c4cc32)c1 ; N#Cc1ccc(-c2ccc(C#N)c(-n3c4ccccc4c4c5c(ccc43)oc3ccccc35)c2)cc1-n1c2ccccc2c2cc3oc4ccccc4c3cc21 ; N#Cc1cc(-c2cccc(-n3c4ccccc4c4c5oc6ccccc6c5ccc43)c2)cc(-n2c3ccccc3c3ccc4c5ccccc5oc4c32)c1]

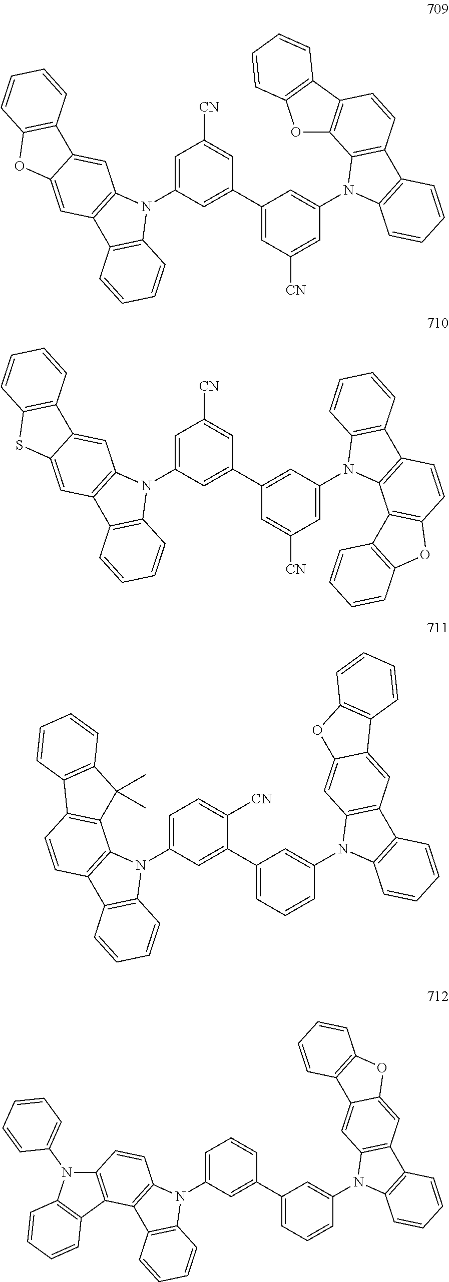 [SMILES: N#Cc1cc(-c2cc(C#N)cc(-n3c4ccccc4c4ccc5c6ccccc6oc5c43)c2)cc(-n2c3ccccc3c3cc4oc5ccccc5c4cc32)c1 ; N#Cc1cc(-c2cc(C#N)cc(-n3c4ccccc4c4ccc5oc6ccccc6c5c43)c2)cc(-n2c3ccccc3c3cc4sc5ccccc5c4cc32)c1 ; CC1(C)c2ccccc2-c2ccc3c4ccccc4n(-c4ccc(C#N)c(-c5cccc(-n6c7ccccc7c7cc8c(cc76)oc6ccccc68)c5)c4)c3c21 ; c1ccc(-n2c3ccccc3c3c4c5ccccc5n(-c5cccc(-c6cccc(-n7c8ccccc8c8cc9oc%10ccccc%10c9cc87)c6)c5)c4ccc32)cc1]

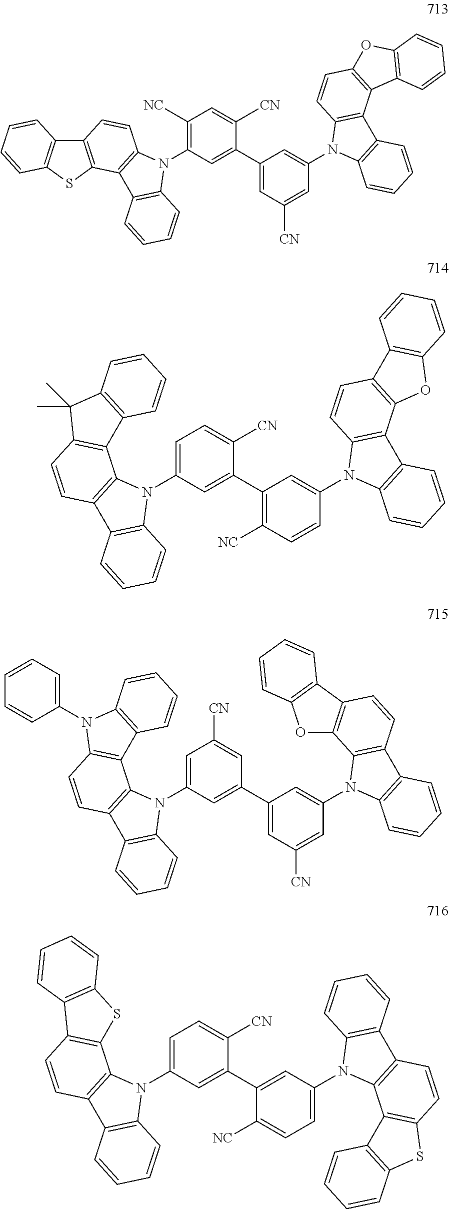 [SMILES: N#Cc1cc(-c2cc(-n3c4ccccc4c4c5sc6ccccc6c5ccc43)c(C#N)cc2C#N)cc(-n2c3ccccc3c3c4c(ccc32)oc2ccccc24)c1 ; CC1(C)c2ccccc2-c2c1ccc1c3ccccc3n(-c3ccc(C#N)c(-c4cc(-n5c6ccccc6c6c7oc8ccccc8c7ccc65)ccc4C#N)c3)c21 ; N#Cc1cc(-c2cc(C#N)cc(-n3c4ccccc4c4ccc5c(c6ccccc6n5-c5ccccc5)c43)c2)cc(-n2c3ccccc3c3ccc4c5ccccc5oc4c32)c1 ; N#Cc1ccc(-n2c3ccccc3c3ccc4c5ccccc5sc4c32)cc1-c1cc(-n2c3ccccc3c3ccc4sc5ccccc5c4c32)ccc1C#N]

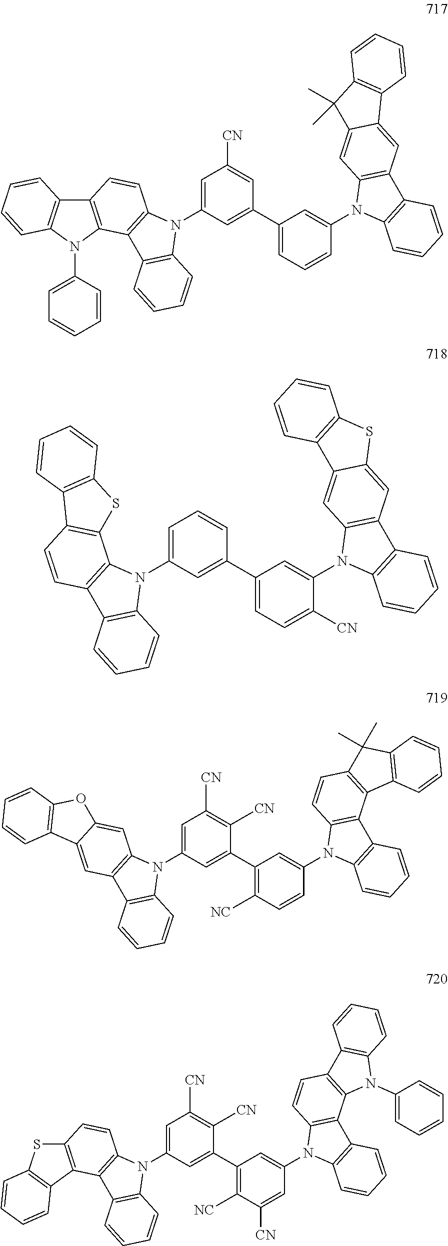 [SMILES: CC1(C)c2ccccc2-c2cc3c4ccccc4n(-c4cccc(-c5cc(C#N)cc(-n6c7ccccc7c7c6ccc6c8ccccc8n(-c8ccccc8)c67)c5)c4)c3cc21 ; N#Cc1ccc(-c2cccc(-n3c4ccccc4c4ccc5c6ccccc6sc5c43)c2)cc1-n1c2ccccc2c2cc3sc4ccccc4c3cc21 ; CC1(C)c2ccccc2-c2c1ccc1c2c2ccccc2n1-c1ccc(C#N)c(-c2cc(-n3c4ccccc4c4cc5c(cc43)oc3ccccc35)cc(C#N)c2C#N)c1 ; N#Cc1cc(-n2c3ccccc3c3c4c(ccc32)sc2ccccc24)cc(-c2cc(-n3c4ccccc4c4c3ccc3c5ccccc5n(-c5ccccc5)c34)cc(C#N)c2C#N)c1C#N]

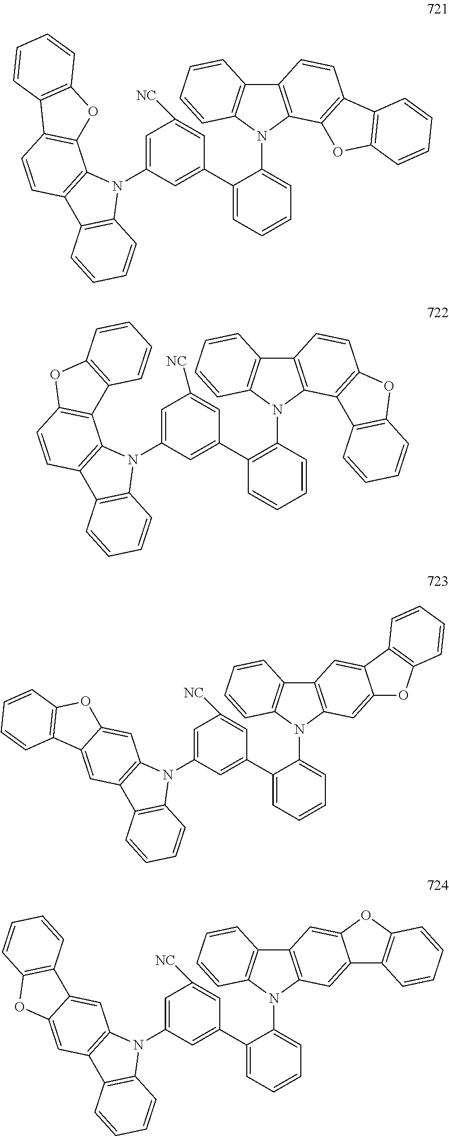 [SMILES: N#Cc1cc(-c2ccccc2-n2c3ccccc3c3ccc4c5ccccc5oc4c32)cc(-n2c3ccccc3c3ccc4c5ccccc5oc4c32)c1 ; N#Cc1cc(-c2ccccc2-n2c3ccccc3c3ccc4oc5ccccc5c4c32)cc(-n2c3ccccc3c3ccc4oc5ccccc5c4c32)c1 ; N#Cc1cc(-c2ccccc2-n2c3ccccc3c3cc4c(cc32)oc2ccccc24)cc(-n2c3ccccc3c3cc4c(cc32)oc2ccccc24)c1 ; N#Cc1cc(-c2ccccc2-n2c3ccccc3c3cc4oc5ccccc5c4cc32)cc(-n2c3ccccc3c3cc4oc5ccccc5c4cc32)c1]

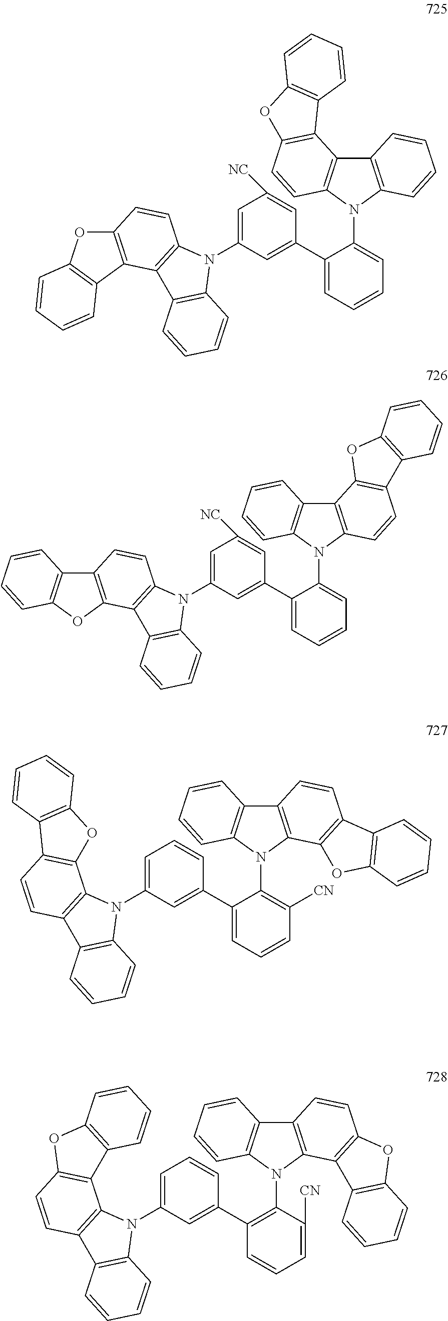 [SMILES: N#Cc1cc(-c2ccccc2-n2c3ccccc3c3c4c(ccc32)oc2ccccc24)cc(-n2c3ccccc3c3c4c(ccc32)oc2ccccc24)c1 ; N#Cc1cc(-c2ccccc2-n2c3ccccc3c3c4oc5ccccc5c4ccc32)cc(-n2c3ccccc3c3c4oc5ccccc5c4ccc32)c1 ; N#Cc1cccc(-c2cccc(-n3c4ccccc4c4ccc5c6ccccc6oc5c43)c2)c1-n1c2ccccc2c2ccc3c4ccccc4oc3c21 ; N#Cc1cccc(-c2cccc(-n3c4ccccc4c4ccc5oc6ccccc6c5c43)c2)c1-n1c2ccccc2c2ccc3oc4ccccc4c3c21]

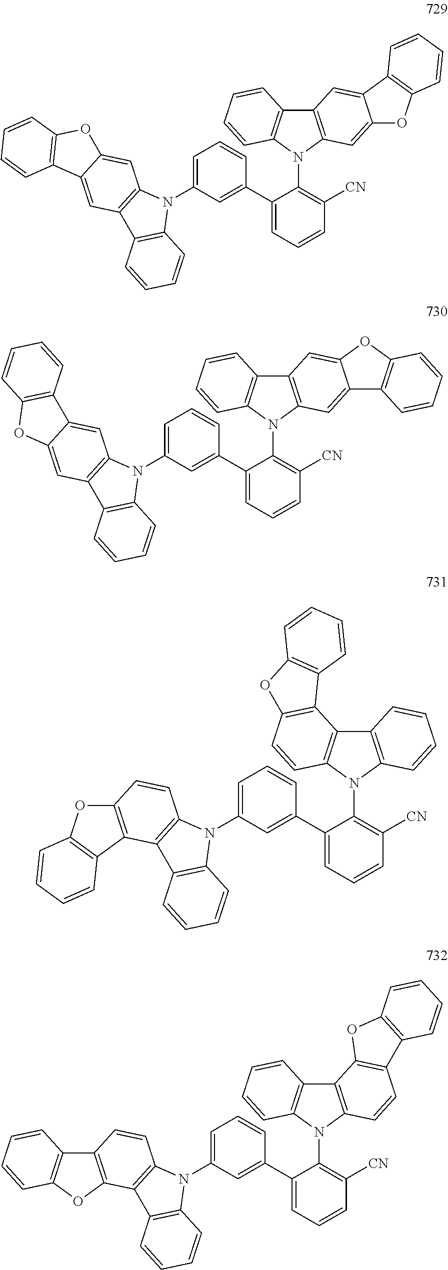 [SMILES: N#Cc1cccc(-c2cccc(-n3c4ccccc4c4cc5c(cc43)oc3ccccc35)c2)c1-n1c2ccccc2c2cc3c(cc21)oc1ccccc13 ; N#Cc1cccc(-c2cccc(-n3c4ccccc4c4cc5oc6ccccc6c5cc43)c2)c1-n1c2ccccc2c2cc3oc4ccccc4c3cc21 ; N#Cc1cccc(-c2cccc(-n3c4ccccc4c4c5c(ccc43)oc3ccccc35)c2)c1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21]

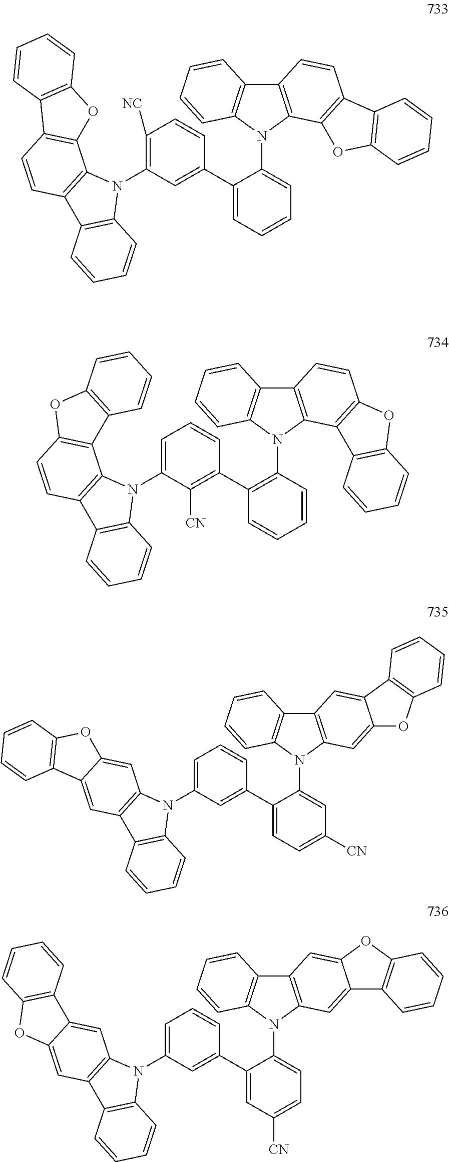 [SMILES: N#Cc1ccc(-c2ccccc2-n2c3ccccc3c3ccc4c5ccccc5oc4c32)cc1-n1c2ccccc2c2ccc3c4ccccc4oc3c21 ; N#Cc1c(-c2ccccc2-n2c3ccccc3c3ccc4oc5ccccc5c4c32)cccc1-n1c2ccccc2c2ccc3oc4ccccc4c3c21 ; N#Cc1ccc(-c2cccc(-n3c4ccccc4c4cc5c(cc43)oc3ccccc35)c2)c(-n2c3ccccc3c3cc4c(cc32)oc2ccccc24)c1 ; N#Cc1ccc(-n2c3ccccc3c3cc4oc5ccccc5c4cc32)c(-c2cccc(-n3c4ccccc4c4cc5oc6ccccc6c5cc43)c2)c1]

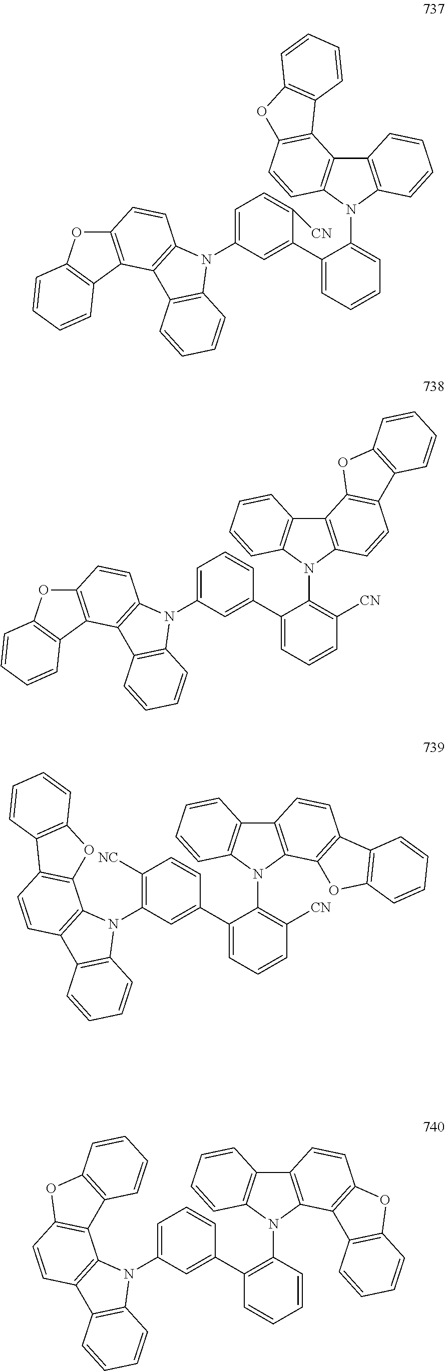 [SMILES: N#Cc1ccc(-n2c3ccccc3c3c4c(ccc32)oc2ccccc24)cc1-c1ccccc1-n1c2ccccc2c2c3c(ccc21)oc1ccccc13 ; N#Cc1cccc(-c2cccc(-n3c4ccccc4c4c5c(ccc43)oc3ccccc35)c2)c1-n1c2ccccc2c2c3oc4ccccc4c3ccc21 ; N#Cc1ccc(-c2cccc(C#N)c2-n2c3ccccc3c3ccc4c5ccccc5oc4c32)cc1-n1c2ccccc2c2ccc3c4ccccc4oc3c21 ; c1cc(-c2ccccc2-n2c3ccccc3c3ccc4oc5ccccc5c4c32)cc(-n2c3ccccc3c3ccc4oc5ccccc5c4c32)c1]

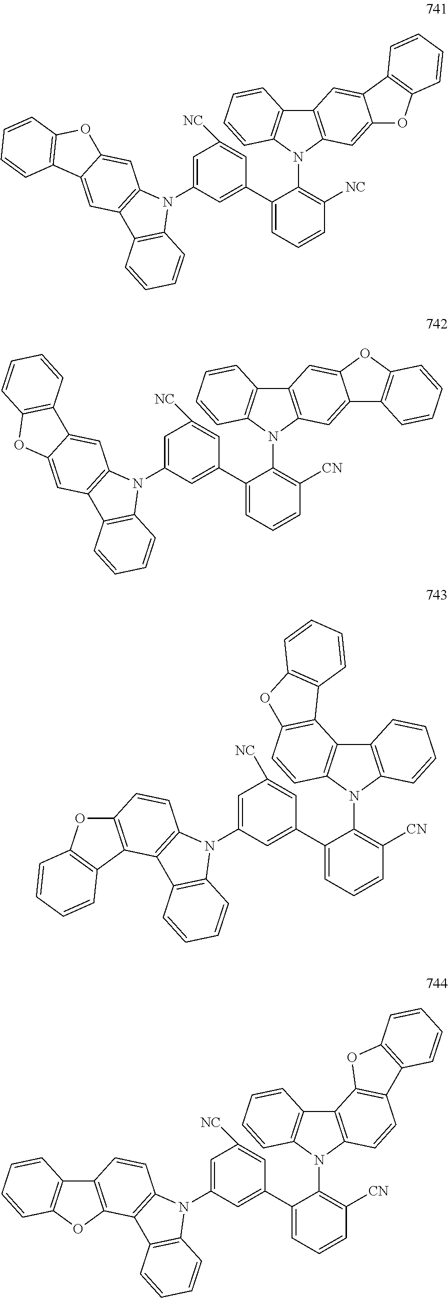

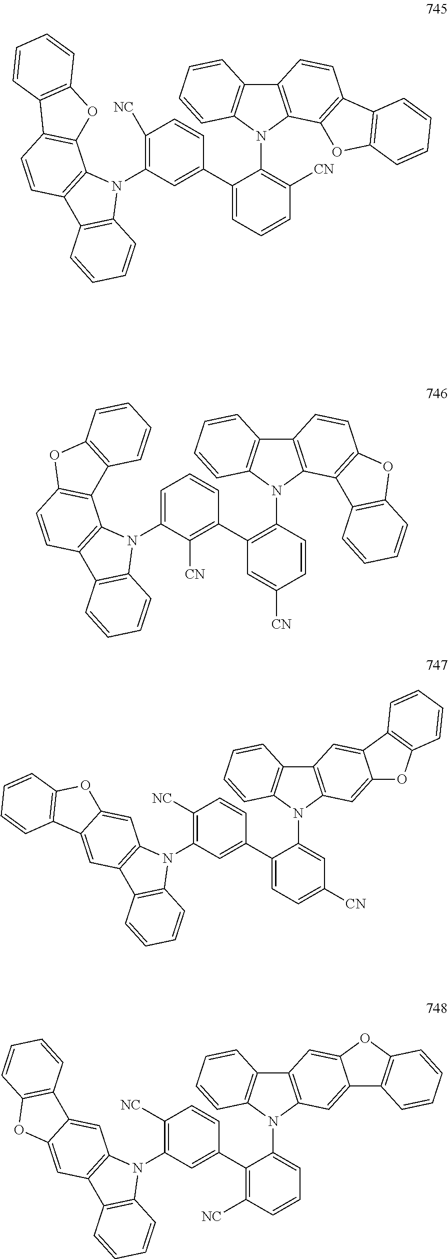 [SMILES: N#Cc1ccc(-c2cccc(C#N)c2-n2c3ccccc3c3ccc4c5ccccc5oc4c32)cc1-n1c2ccccc2c2ccc3c4ccccc4oc3c21 ; N#Cc1ccc(-n2c3ccccc3c3ccc4oc5ccccc5c4c32)c(-c2cccc(-n3c4ccccc4c4ccc5oc6ccccc6c5c43)c2C#N)c1 ; N#Cc1ccc(-c2ccc(C#N)c(-n3c4ccccc4c4cc5c(cc43)oc3ccccc35)c2)c(-n2c3ccccc3c3cc4c(cc32)oc2ccccc24)c1 ; N#Cc1ccc(-c2c(C#N)cccc2-n2c3ccccc3c3cc4oc5ccccc5c4cc32)cc1-n1c2ccccc2c2cc3oc4ccccc4c3cc21]

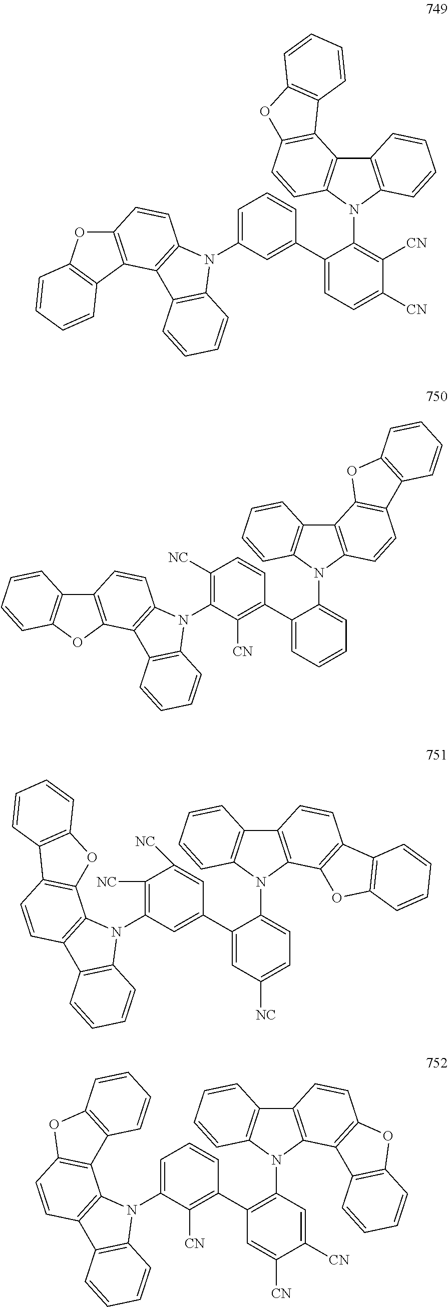

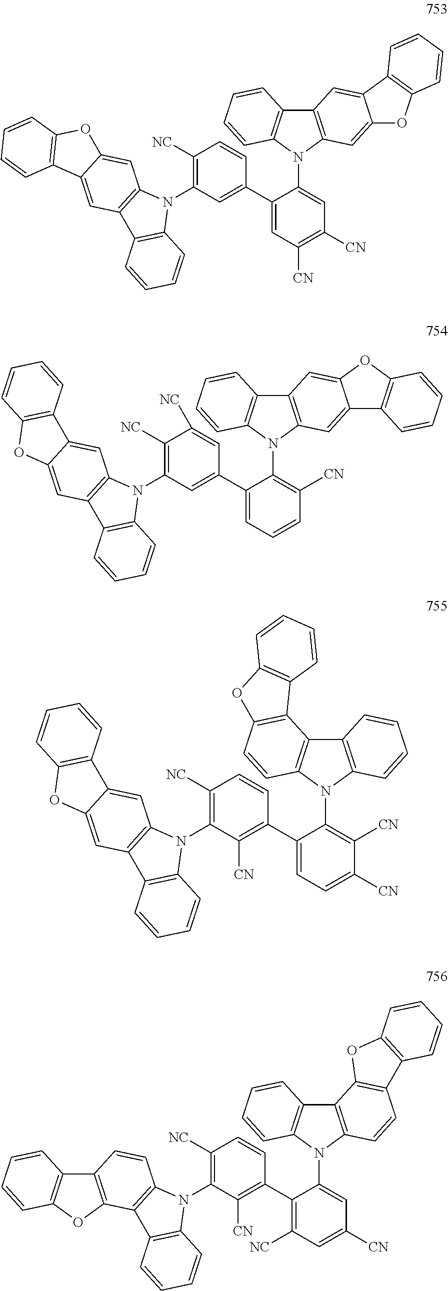 [SMILES: N#Cc1cc(-c2ccc(C#N)c(-n3c4ccccc4c4cc5c(cc43)oc3ccccc35)c2)c(-n2c3ccccc3c3cc4c(cc32)oc2ccccc24)cc1C#N ; N#Cc1cc(-c2cccc(C#N)c2-n2c3ccccc3c3cc4oc5ccccc5c4cc32)cc(-n2c3ccccc3c3cc4oc5ccccc5c4cc32)c1C#N ; N#Cc1ccc(-c2ccc(C#N)c(-n3c4ccccc4c4cc5oc6ccccc6c5cc43)c2C#N)c(-n2c3ccccc3c3c4c(ccc32)oc2ccccc24)c1C#N ; N#Cc1cc(C#N)c(-c2ccc(C#N)c(-n3c4ccccc4c4c5oc6ccccc6c5ccc43)c2C#N)c(-n2c3ccccc3c3c4oc5ccccc5c4ccc32)c1]

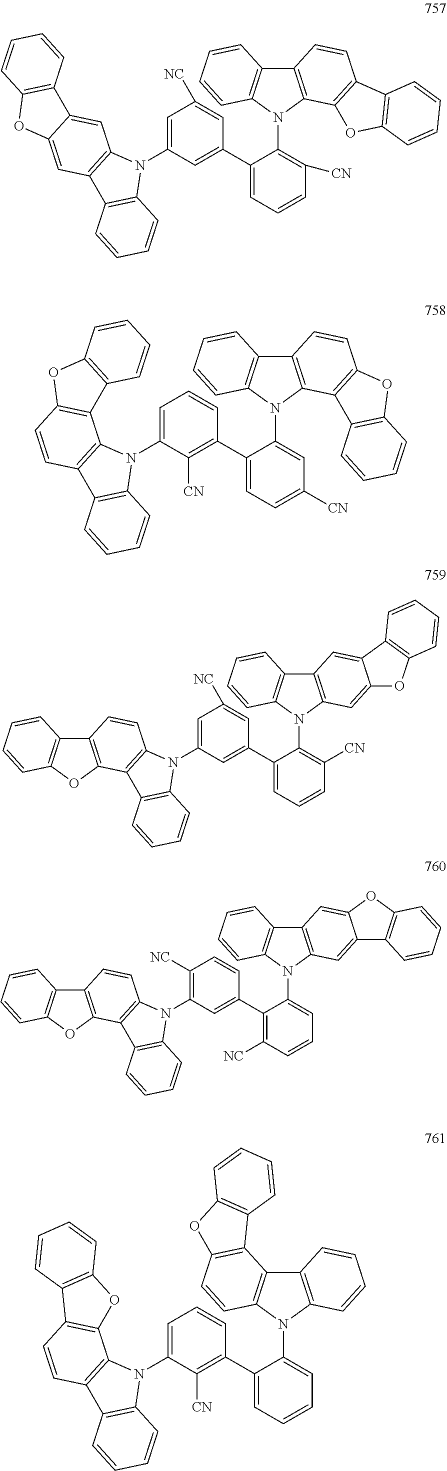 [SMILES: N#Cc1cc(-c2cccc(C#N)c2-n2c3ccccc3c3ccc4c5ccccc5oc4c32)cc(-n2c3ccccc3c3cc4oc5ccccc5c4cc32)c1 ; N#Cc1ccc(-c2cccc(-n3c4ccccc4c4ccc5oc6ccccc6c5c43)c2C#N)c(-n2c3ccccc3c3ccc4oc5ccccc5c4c32)c1 ; N#Cc1cc(-c2cccc(C#N)c2-n2c3ccccc3c3cc4c(cc32)oc2ccccc24)cc(-n2c3ccccc3c3c4oc5ccccc5c4ccc32)c1 ; N#Cc1ccc(-c2c(C#N)cccc2-n2c3ccccc3c3cc4oc5ccccc5c4cc32)cc1-n1c2ccccc2c2c3oc4ccccc4c3ccc21 ; N#Cc1c(-c2ccccc2-n2c3ccccc3c3c4c(ccc32)oc2ccccc24)cccc1-n1c2ccccc2c2ccc3c4ccccc4oc3c21]

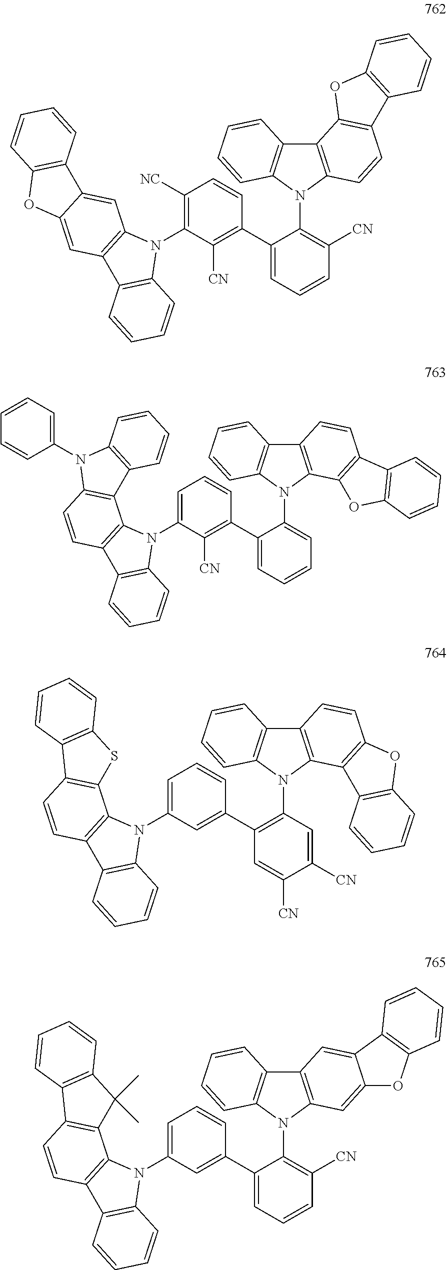 [SMILES: N#Cc1ccc(-c2cccc(C#N)c2-n2c3ccccc3c3c4oc5ccccc5c4ccc32)c(C#N)c1-n1c2ccccc2c2cc3oc4ccccc4c3cc21 ; N#Cc1c(-c2ccccc2-n2c3ccccc3c3ccc4c5ccccc5oc4c32)cccc1-n1c2ccccc2c2ccc3c(c4ccccc4n3-c3ccccc3)c21 ; N#Cc1cc(-c2cccc(-n3c4ccccc4c4ccc5c6ccccc6sc5c43)c2)c(-n2c3ccccc3c3ccc4oc5ccccc5c4c32)cc1C#N ; CC1(C)c2ccccc2-c2ccc3c4ccccc4n(-c4cccc(-c5cccc(C#N)c5-n5c6ccccc6c6cc7c(cc65)oc5ccccc57)c4)c3c21]

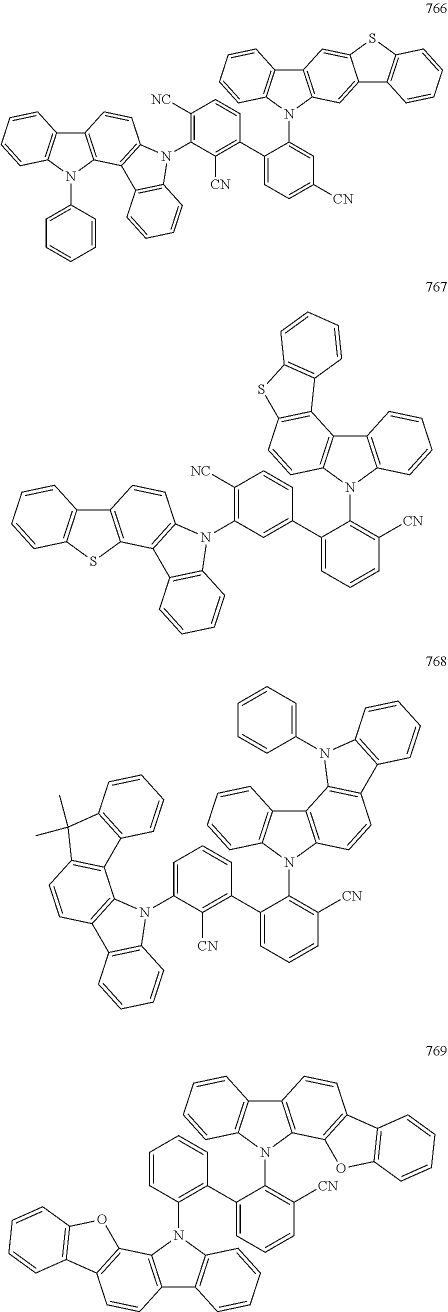 [SMILES: N#Cc1ccc(-c2ccc(C#N)c(-n3c4ccccc4c4c3ccc3c5ccccc5n(-c5ccccc5)c34)c2C#N)c(-n2c3ccccc3c3cc4sc5ccccc5c4cc32)c1 ; N#Cc1ccc(-c2cccc(C#N)c2-n2c3ccccc3c3c4c(ccc32)sc2ccccc24)cc1-n1c2ccccc2c2c3sc4ccccc4c3ccc21 ; CC1(C)c2ccccc2-c2c1ccc1c3ccccc3n(-c3cccc(-c4cccc(C#N)c4-n4c5ccccc5c5c4ccc4c6ccccc6n(-c6ccccc6)c45)c3C#N)c21 ; N#Cc1cccc(-c2ccccc2-n2c3ccccc3c3ccc4c5ccccc5oc4c32)c1-n1c2ccccc2c2ccc3c4ccccc4oc3c21]

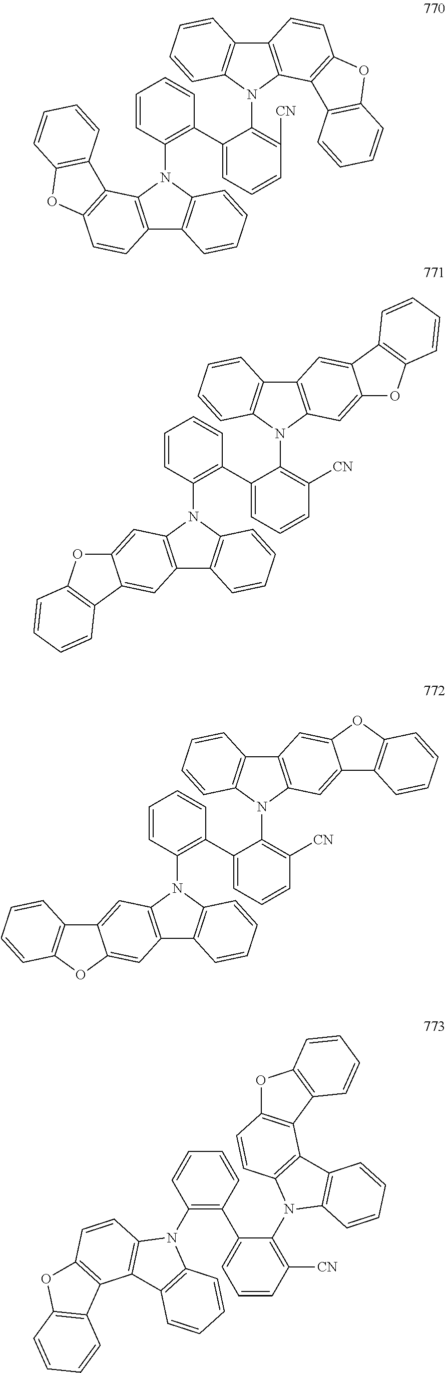 [SMILES: N#Cc1cccc(-c2ccccc2-n2c3ccccc3c3ccc4oc5ccccc5c4c32)c1-n1c2ccccc2c2ccc3oc4ccccc4c3c21 ; N#Cc1cccc(-c2ccccc2-n2c3ccccc3c3cc4c(cc32)oc2ccccc24)c1-n1c2ccccc2c2cc3c(cc21)oc1ccccc13 ; N#Cc1cccc(-c2ccccc2-n2c3ccccc3c3cc4oc5ccccc5c4cc32)c1-n1c2ccccc2c2cc3oc4ccccc4c3cc21 ; N#Cc1cccc(-c2ccccc2-n2c3ccccc3c3c4c(ccc32)oc2ccccc24)c1-n1c2ccccc2c2c3c(ccc21)oc1ccccc13]

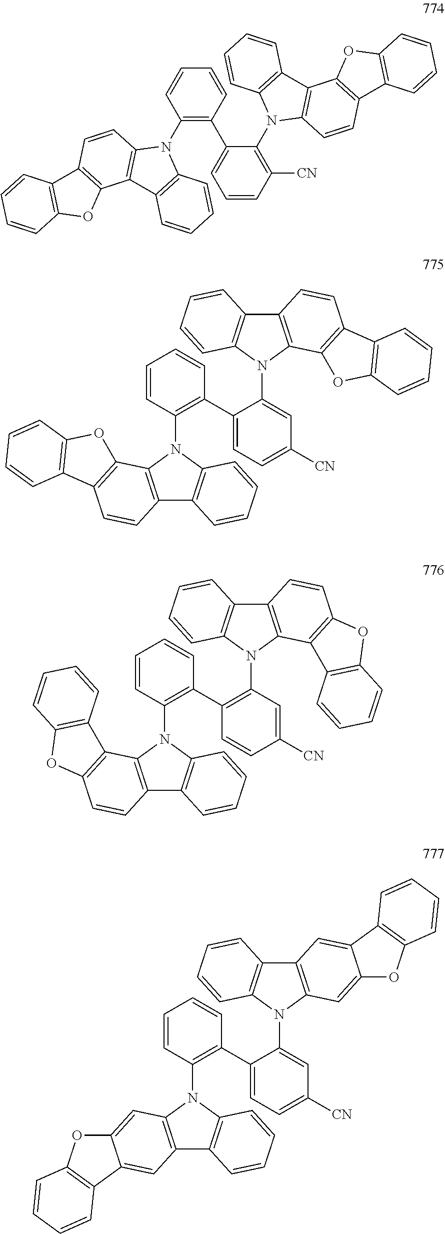 [SMILES: N#Cc1cccc(-c2ccccc2-n2c3ccccc3c3c4oc5ccccc5c4ccc32)c1-n1c2ccccc2c2c3oc4ccccc4c3ccc21 ; N#Cc1ccc(-c2ccccc2-n2c3ccccc3c3ccc4c5ccccc5oc4c32)c(-n2c3ccccc3c3ccc4c5ccccc5oc4c32)c1 ; N#Cc1ccc(-c2ccccc2-n2c3ccccc3c3ccc4oc5ccccc5c4c32)c(-n2c3ccccc3c3ccc4oc5ccccc5c4c32)c1 ; N#Cc1ccc(-c2ccccc2-n2c3ccccc3c3cc4c(cc32)oc2ccccc24)c(-n2c3ccccc3c3cc4c(cc32)oc2ccccc24)c1]

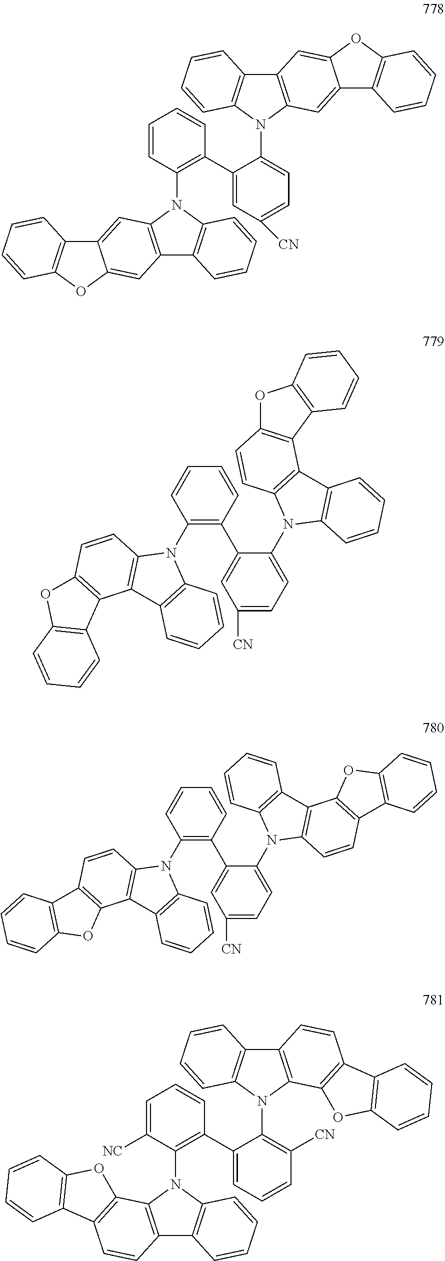 [SMILES: N#Cc1ccc(-n2c3ccccc3c3cc4oc5ccccc5c4cc32)c(-c2ccccc2-n2c3ccccc3c3cc4oc5ccccc5c4cc32)c1 ; N#Cc1ccc(-n2c3ccccc3c3c4c(ccc32)oc2ccccc24)c(-c2ccccc2-n2c3ccccc3c3c4c(ccc32)oc2ccccc24)c1 ; N#Cc1ccc(-n2c3ccccc3c3c4oc5ccccc5c4ccc32)c(-c2ccccc2-n2c3ccccc3c3c4oc5ccccc5c4ccc32)c1 ; N#Cc1cccc(-c2cccc(C#N)c2-n2c3ccccc3c3ccc4c5ccccc5oc4c32)c1-n1c2ccccc2c2ccc3c4ccccc4oc3c21]

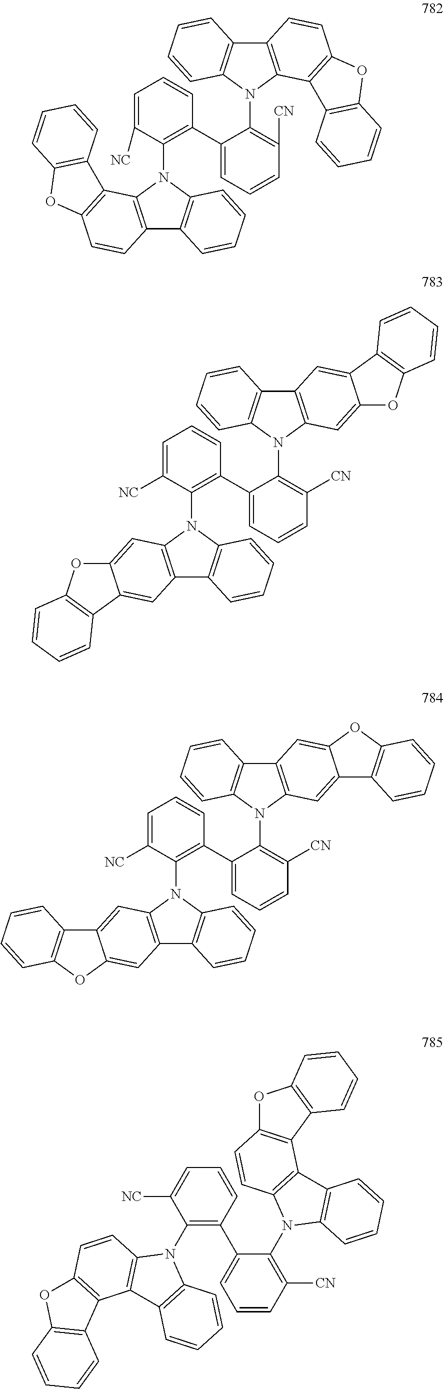 [SMILES: N#Cc1cccc(-c2cccc(C#N)c2-n2c3ccccc3c3ccc4oc5ccccc5c4c32)c1-n1c2ccccc2c2ccc3oc4ccccc4c3c21 ; N#Cc1cccc(-c2cccc(C#N)c2-n2c3ccccc3c3cc4c(cc32)oc2ccccc24)c1-n1c2ccccc2c2cc3c(cc21)oc1ccccc13 ; N#Cc1cccc(-c2cccc(C#N)c2-n2c3ccccc3c3cc4oc5ccccc5c4cc32)c1-n1c2ccccc2c2cc3oc4ccccc4c3cc21 ; N#Cc1cccc(-c2cccc(C#N)c2-n2c3ccccc3c3c4c(ccc32)oc2ccccc24)c1-n1c2ccccc2c2c3c(ccc21)oc1ccccc13]

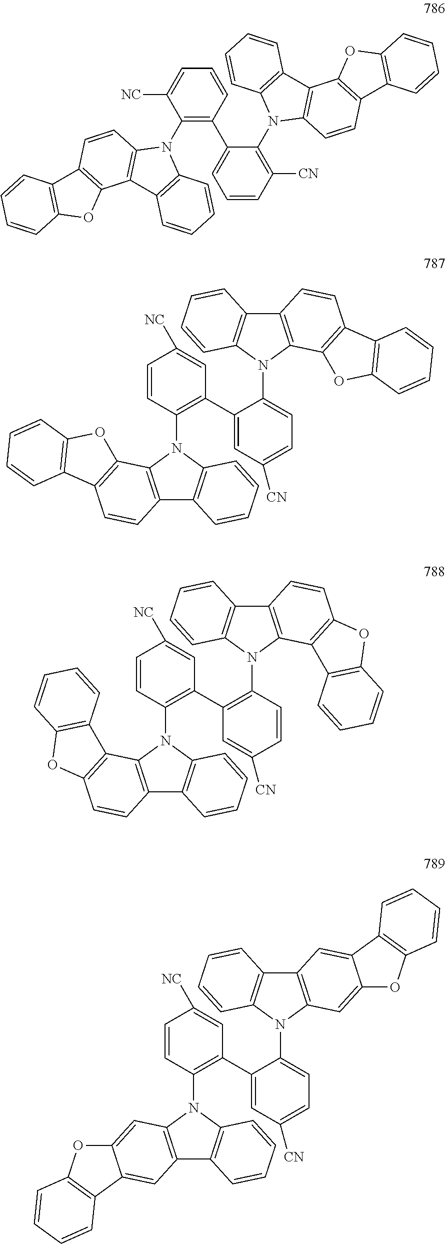 [SMILES: N#Cc1cccc(-c2cccc(C#N)c2-n2c3ccccc3c3c4oc5ccccc5c4ccc32)c1-n1c2ccccc2c2c3oc4ccccc4c3ccc21 ; N#Cc1ccc(-n2c3ccccc3c3ccc4c5ccccc5oc4c32)c(-c2cc(C#N)ccc2-n2c3ccccc3c3ccc4c5ccccc5oc4c32)c1 ; N#Cc1ccc(-n2c3ccccc3c3ccc4oc5ccccc5c4c32)c(-c2cc(C#N)ccc2-n2c3ccccc3c3ccc4oc5ccccc5c4c32)c1 ; N#Cc1ccc(-n2c3ccccc3c3cc4c(cc32)oc2ccccc24)c(-c2cc(C#N)ccc2-n2c3ccccc3c3cc4c(cc32)oc2ccccc24)c1]

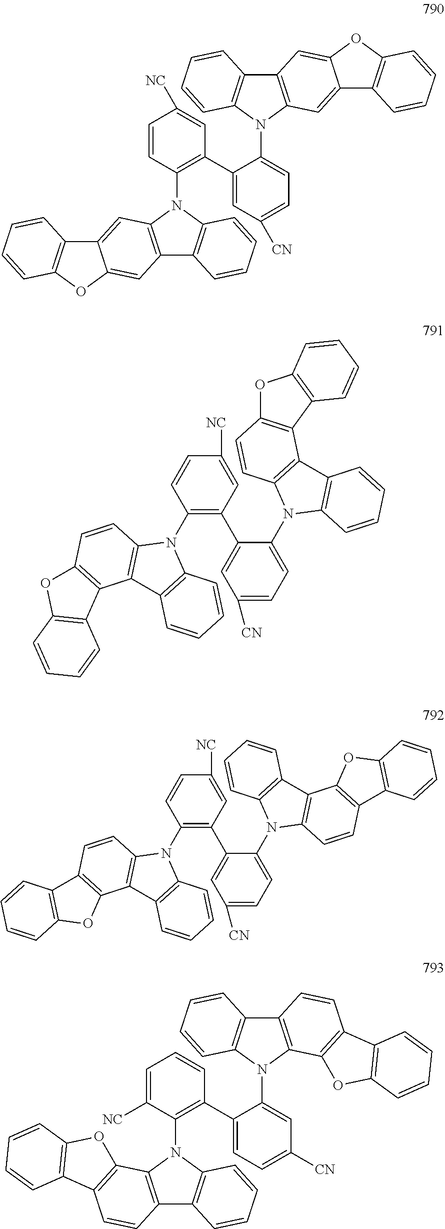 [SMILES: N#Cc1ccc(-n2c3ccccc3c3cc4oc5ccccc5c4cc32)c(-c2cc(C#N)ccc2-n2c3ccccc3c3cc4oc5ccccc5c4cc32)c1 ; N#Cc1ccc(-n2c3ccccc3c3c4c(ccc32)oc2ccccc24)c(-c2cc(C#N)ccc2-n2c3ccccc3c3c4c(ccc32)oc2ccccc24)c1 ; N#Cc1ccc(-n2c3ccccc3c3c4oc5ccccc5c4ccc32)c(-c2cc(C#N)ccc2-n2c3ccccc3c3c4oc5ccccc5c4ccc32)c1 ; N#Cc1ccc(-c2cccc(C#N)c2-n2c3ccccc3c3ccc4c5ccccc5oc4c32)c(-n2c3ccccc3c3ccc4c5ccccc5oc4c32)c1]

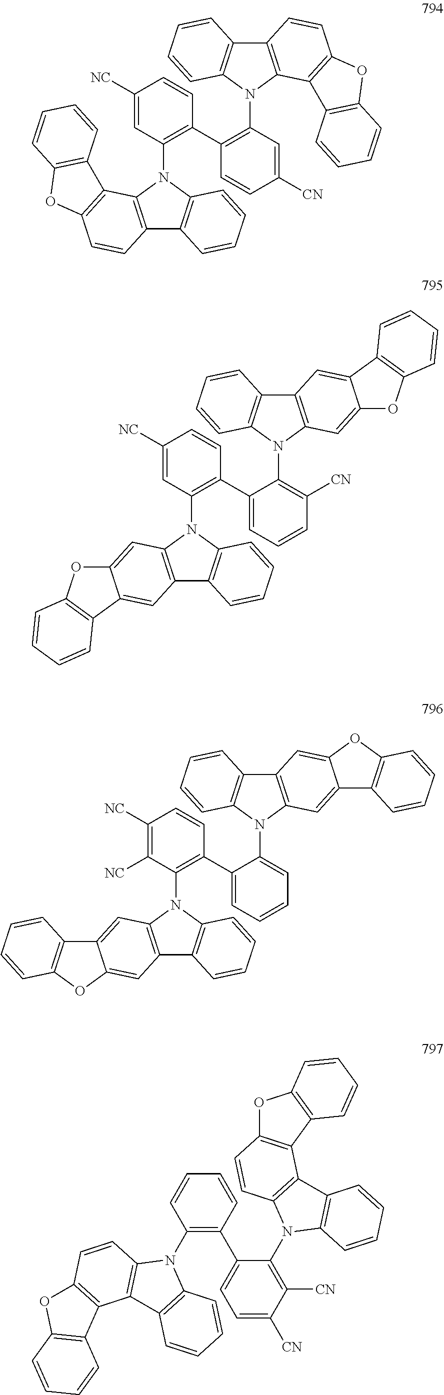 [SMILES: N#Cc1ccc(-c2ccc(C#N)cc2-n2c3ccccc3c3ccc4oc5ccccc5c4c32)c(-n2c3ccccc3c3ccc4oc5ccccc5c4c32)c1 ; N#Cc1ccc(-c2cccc(C#N)c2-n2c3ccccc3c3cc4c(cc32)oc2ccccc24)c(-n2c3ccccc3c3cc4c(cc32)oc2ccccc24)c1 ; N#Cc1ccc(-c2ccccc2-n2c3ccccc3c3cc4oc5ccccc5c4cc32)c(-n2c3ccccc3c3cc4oc5ccccc5c4cc32)c1C#N ; N#Cc1ccc(-c2ccccc2-n2c3ccccc3c3c4c(ccc32)oc2ccccc24)c(-n2c3ccccc3c3c4c(ccc32)oc2ccccc24)c1C#N]

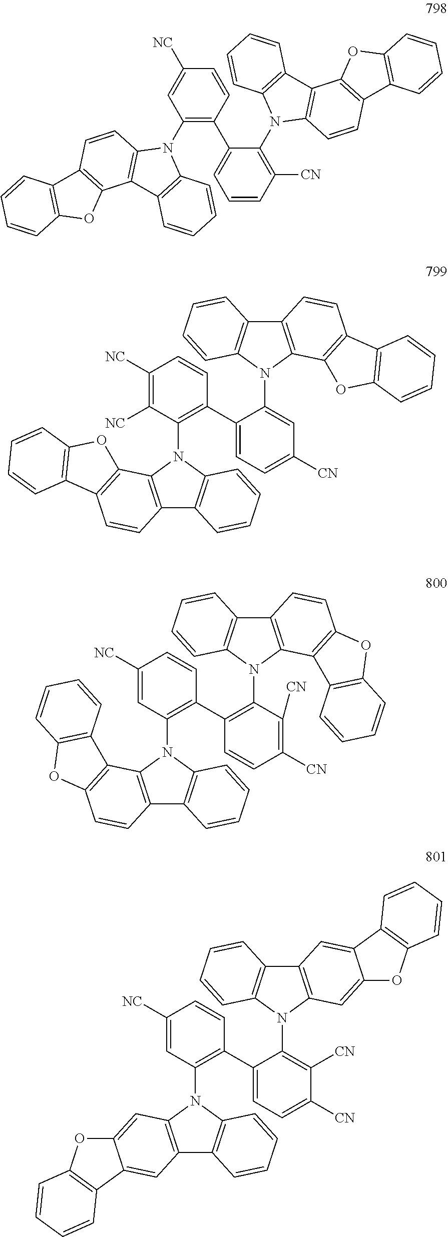 [SMILES: N#Cc1ccc(-c2cccc(C#N)c2-n2c3ccccc3c3c4oc5ccccc5c4ccc32)c(-n2c3ccccc3c3c4oc5ccccc5c4ccc32)c1 ; N#Cc1ccc(-c2ccc(C#N)c(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)c(C#N)c2-n2c3ccccc3c3ccc4oc5ccccc5c4c32)c(-n2c3ccccc3c3ccc4oc5ccccc5c4c32)c1 ; N#Cc1ccc(-c2ccc(C#N)c(C#N)c2-n2c3ccccc3c3cc4c(cc32)oc2ccccc24)c(-n2c3ccccc3c3cc4c(cc32)oc2ccccc24)c1]

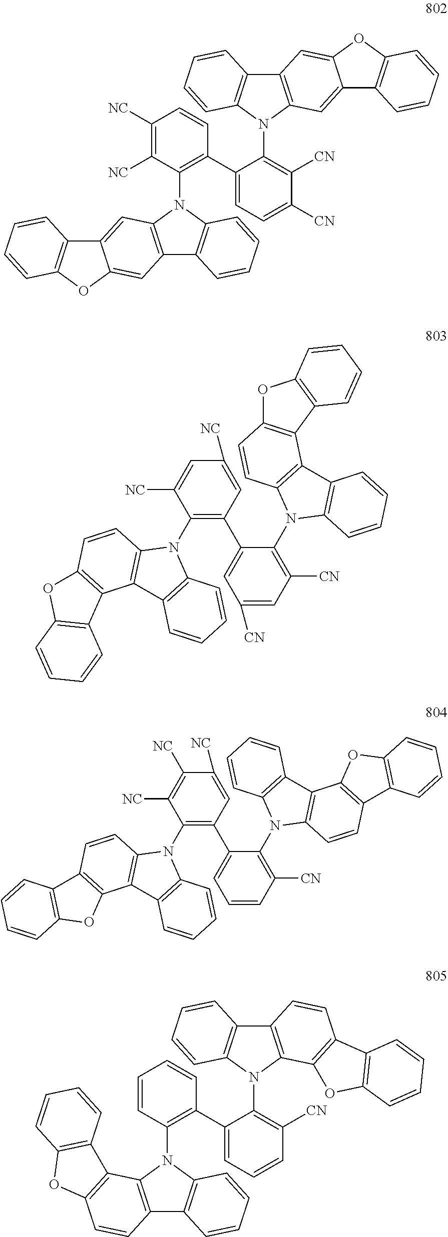 [SMILES: N#Cc1ccc(-c2ccc(C#N)c(C#N)c2-n2c3ccccc3c3cc4oc5ccccc5c4cc32)c(-n2c3ccccc3c3cc4oc5ccccc5c4cc32)c1C#N ; N#Cc1cc(C#N)c(-n2c3ccccc3c3c4c(ccc32)oc2ccccc24)c(-c2cc(C#N)cc(C#N)c2-n2c3ccccc3c3c4c(ccc32)oc2ccccc24)c1 ; N#Cc1cc(-c2cccc(C#N)c2-n2c3ccccc3c3c4oc5ccccc5c4ccc32)c(-n2c3ccccc3c3c4oc5ccccc5c4ccc32)c(C#N)c1C#N ; N#Cc1cccc(-c2ccccc2-n2c3ccccc3c3ccc4oc5ccccc5c4c32)c1-n1c2ccccc2c2ccc3c4ccccc4oc3c21]

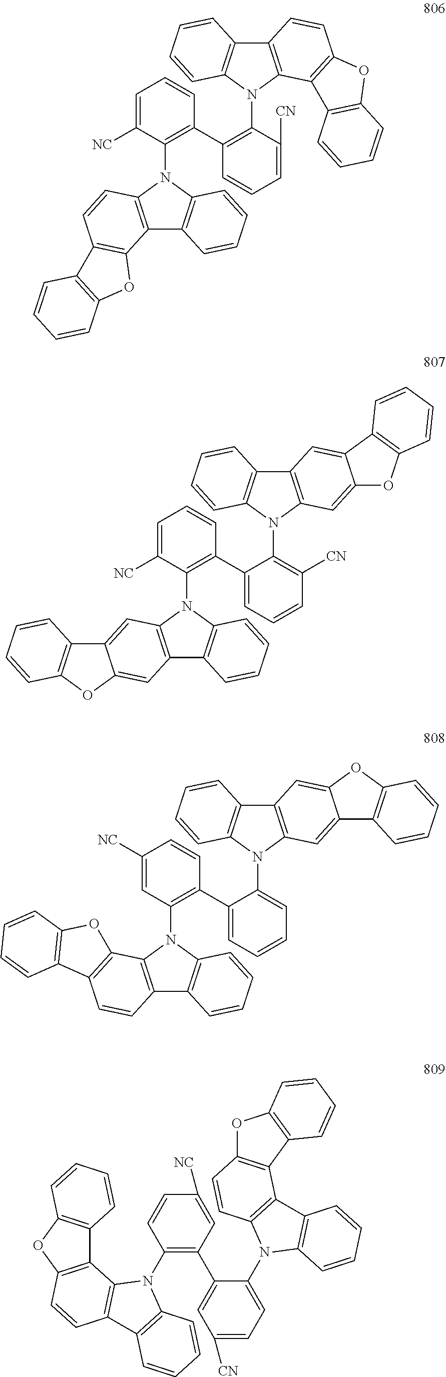 [SMILES: N#Cc1cccc(-c2cccc(C#N)c2-n2c3ccccc3c3ccc4oc5ccccc5c4c32)c1-n1c2ccccc2c2c3oc4ccccc4c3ccc21 ; N#Cc1cccc(-c2cccc(C#N)c2-n2c3ccccc3c3cc4oc5ccccc5c4cc32)c1-n1c2ccccc2c2cc3c(cc21)oc1ccccc13 ; N#Cc1ccc(-c2ccccc2-n2c3ccccc3c3cc4oc5ccccc5c4cc32)c(-n2c3ccccc3c3ccc4c5ccccc5oc4c32)c1 ; N#Cc1ccc(-n2c3ccccc3c3c4c(ccc32)oc2ccccc24)c(-c2cc(C#N)ccc2-n2c3ccccc3c3ccc4oc5ccccc5c4c32)c1]

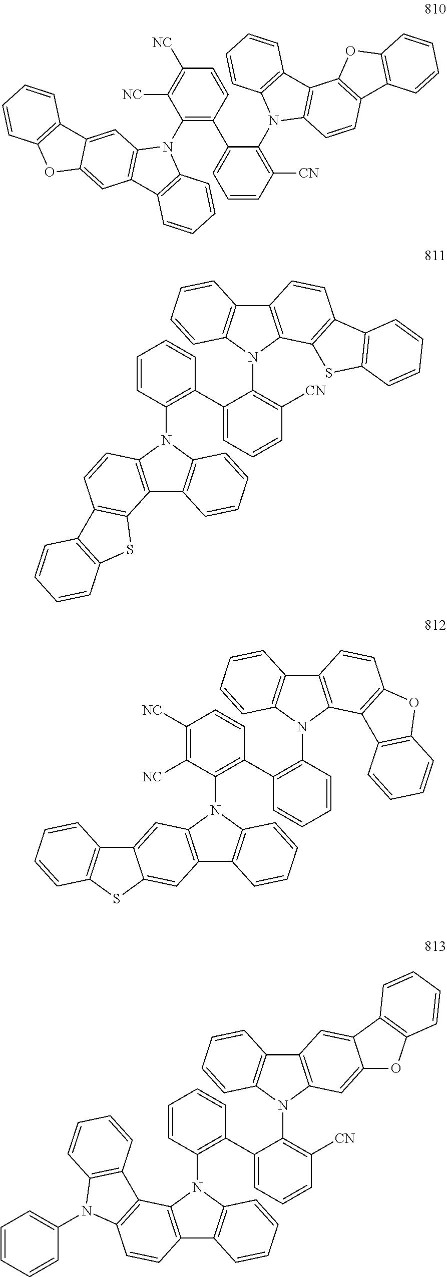 [SMILES: N#Cc1ccc(-c2cccc(C#N)c2-n2c3ccccc3c3c4oc5ccccc5c4ccc32)c(-n2c3ccccc3c3cc4oc5ccccc5c4cc32)c1C#N ; N#Cc1cccc(-c2ccccc2-n2c3ccccc3c3c4sc5ccccc5c4ccc32)c1-n1c2ccccc2c2ccc3c4ccccc4sc3c21 ; N#Cc1ccc(-c2ccccc2-n2c3ccccc3c3ccc4oc5ccccc5c4c32)c(-n2c3ccccc3c3cc4sc5ccccc5c4cc32)c1C#N ; N#Cc1cccc(-c2ccccc2-n2c3ccccc3c3ccc4c(c5ccccc5n4-c4ccccc4)c32)c1-n1c2ccccc2c2cc3c(cc21)oc1ccccc13]

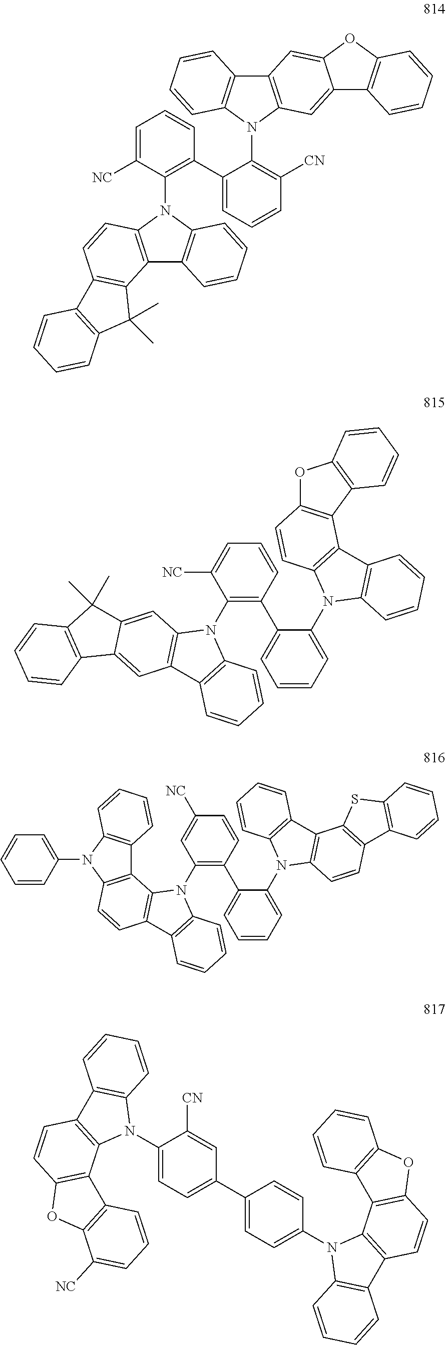 [SMILES: CC1(C)c2ccccc2-c2ccc3c(c21)c1ccccc1n3-c1c(C#N)cccc1-c1cccc(C#N)c1-n1c2ccccc2c2cc3oc4ccccc4c3cc21 ; CC1(C)c2ccccc2-c2cc3c4ccccc4n(-c4c(C#N)cccc4-c4ccccc4-n4c5ccccc5c5c6c(ccc54)oc4ccccc46)c3cc21 ; N#Cc1ccc(-c2ccccc2-n2c3ccccc3c3c4sc5ccccc5c4ccc32)c(-n2c3ccccc3c3ccc4c(c5ccccc5n4-c4ccccc4)c32)c1 ; N#Cc1cc(-c2ccc(-n3c4ccccc4c4ccc5oc6ccccc6c5c43)cc2)ccc1-n1c2ccccc2c2ccc3oc4c(C#N)cccc4c3c21]

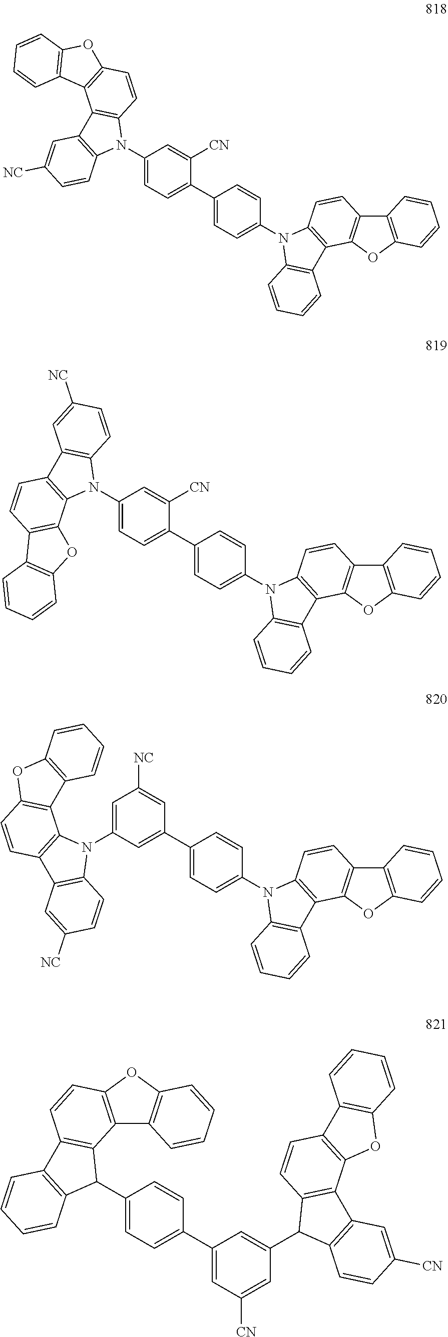

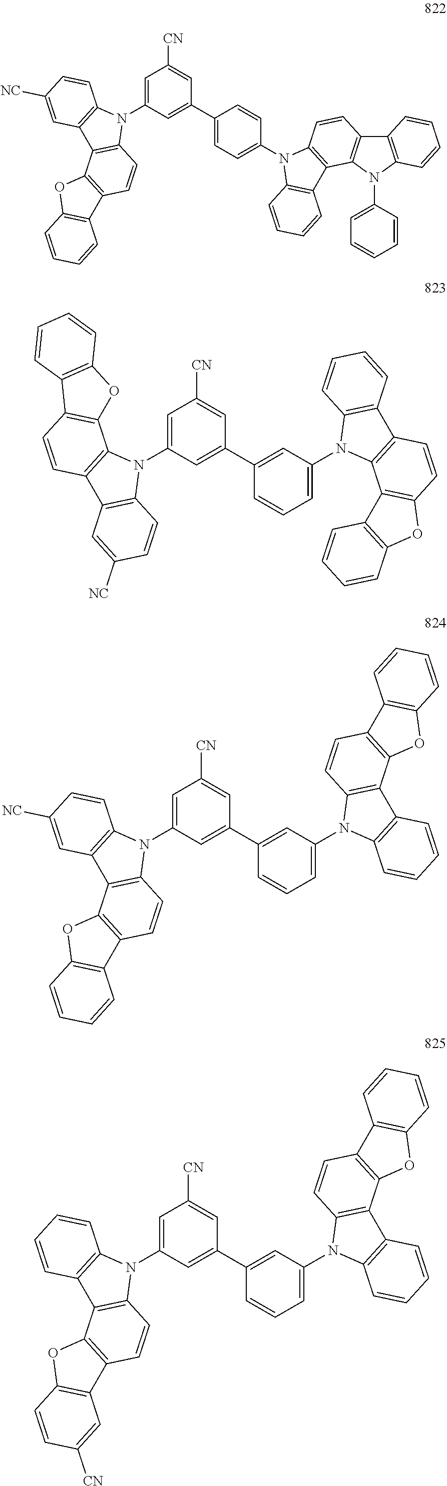 [SMILES: N#Cc1cc(-c2ccc(-n3c4ccccc4c4c3ccc3c5ccccc5n(-c5ccccc5)c34)cc2)cc(-n2c3ccc(C#N)cc3c3c4oc5ccccc5c4ccc32)c1 ; N#Cc1cc(-c2cccc(-n3c4ccccc4c4ccc5oc6ccccc6c5c43)c2)cc(-n2c3ccc(C#N)cc3c3ccc4c5ccccc5oc4c32)c1 ; N#Cc1cc(-c2cccc(-n3c4ccccc4c4c5oc6ccccc6c5ccc43)c2)cc(-n2c3ccc(C#N)cc3c3c4oc5ccccc5c4ccc32)c1 ; N#Cc1cc(-c2cccc(-n3c4ccccc4c4c5oc6ccccc6c5ccc43)c2)cc(-n2c3ccccc3c3c4oc5ccc(C#N)cc5c4ccc32)c1]

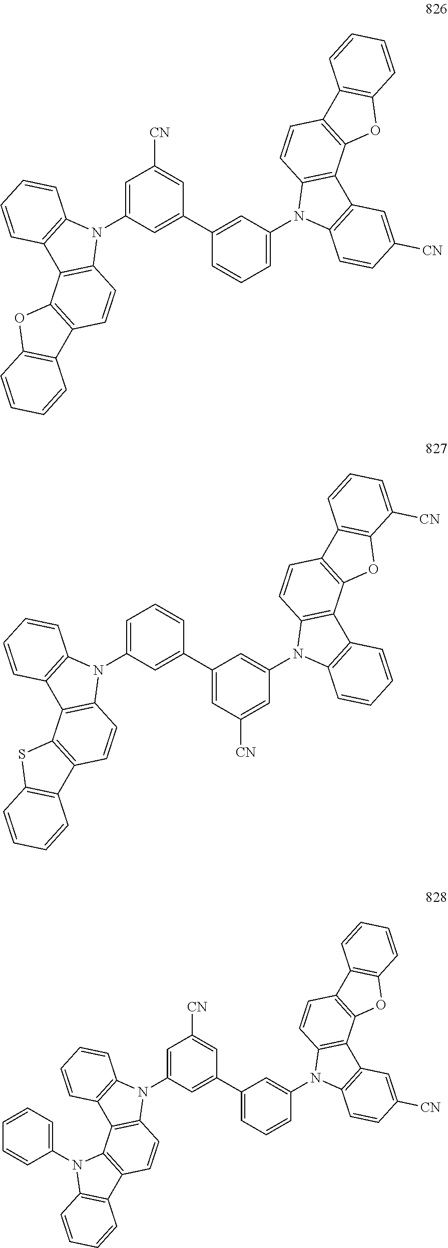 [SMILES: N#Cc1cc(-c2cccc(-n3c4ccc(C#N)cc4c4c5oc6ccccc6c5ccc43)c2)cc(-n2c3ccccc3c3c4oc5ccccc5c4ccc32)c1 ; N#Cc1cc(-c2cccc(-n3c4ccccc4c4c5sc6ccccc6c5ccc43)c2)cc(-n2c3ccccc3c3c4oc5c(C#N)cccc5c4ccc32)c1 ; N#Cc1cc(-c2cccc(-n3c4ccc(C#N)cc4c4c5oc6ccccc6c5ccc43)c2)cc(-n2c3ccccc3c3c2ccc2c4ccccc4n(-c4ccccc4)c23)c1]

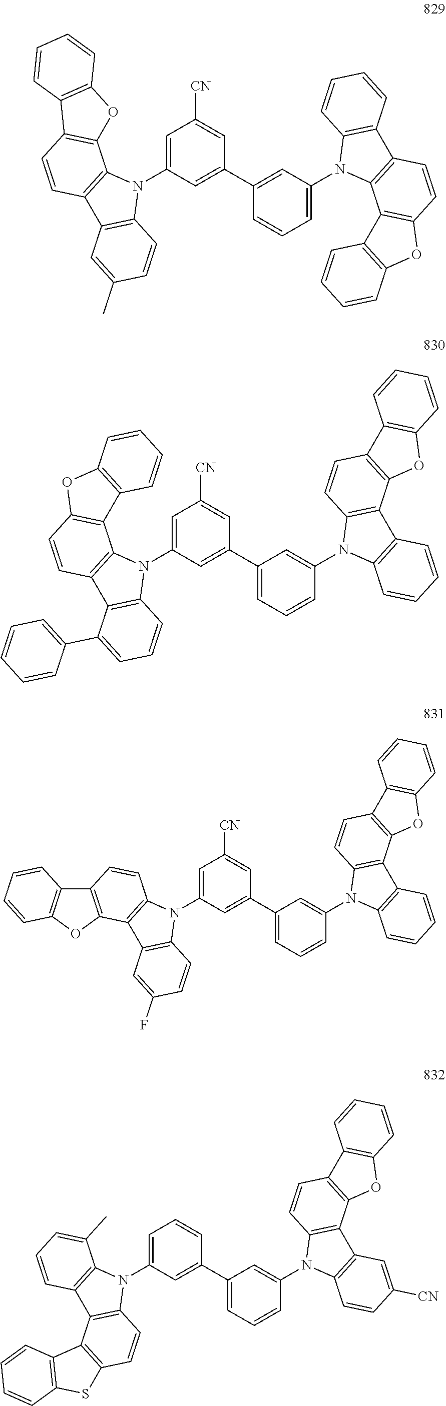 [SMILES: Cc1ccc2c(c1)c1ccc3c4ccccc4oc3c1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3cccc(-c4ccccc4)c3c3ccc4oc5ccccc5c4c32)c1 ; N#Cc1cc(-c2cccc(-n3c4ccccc4c4c5oc6ccccc6c5ccc43)c2)cc(-n2c3ccc(F)cc3c3c4oc5ccccc5c4ccc32)c1 ; Cc1cccc2c3c4c(ccc3n(-c3cccc(-c5cccc(-n6c7ccc(C#N)cc7c7c8oc9ccccc9c8ccc76)c5)c3)c12)sc1ccccc14]

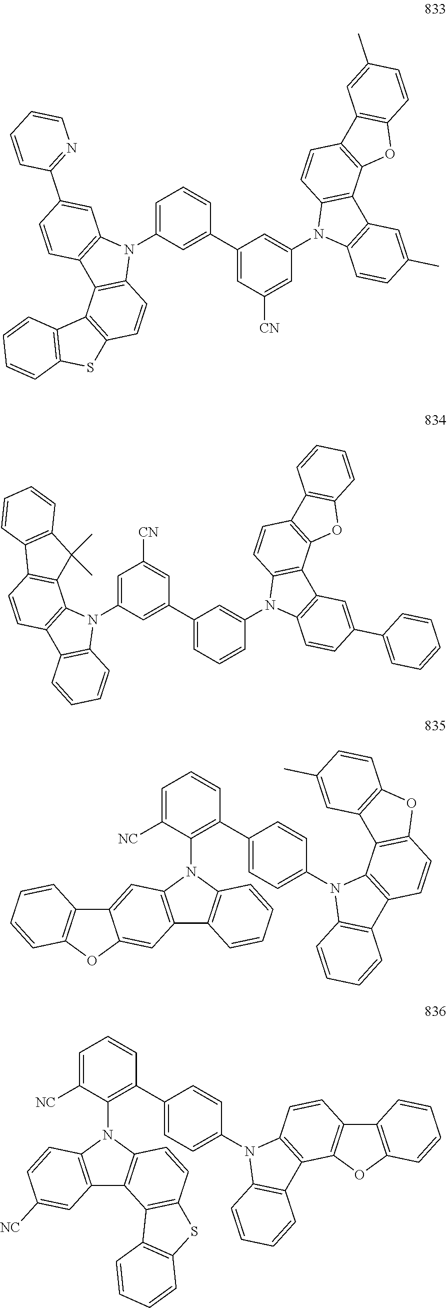 [SMILES: Cc1ccc2oc3c(ccc4c3c3cc(C)ccc3n4-c3cc(C#N)cc(-c4cccc(-n5c6cc(-c7ccccn7)ccc6c6c7c(ccc65)sc5ccccc57)c4)c3)c2c1 ; CC1(C)c2ccccc2-c2ccc3c4ccccc4n(-c4cc(C#N)cc(-c5cccc(-n6c7ccc(-c8ccccc8)cc7c7c8oc9ccccc9c8ccc76)c5)c4)c3c21 ; Cc1ccc2oc3ccc4c5ccccc5n(-c5ccc(-c6cccc(C#N)c6-n6c7ccccc7c7cc8oc9ccccc9c8cc76)cc5)c4c3c2c1 ; N#Cc1ccc2c(c1)c1c3c(ccc1n2-c1c(C#N)cccc1-c1ccc(-n2c4ccccc4c4c5oc6ccccc6c5ccc42)cc1)sc1ccccc13]

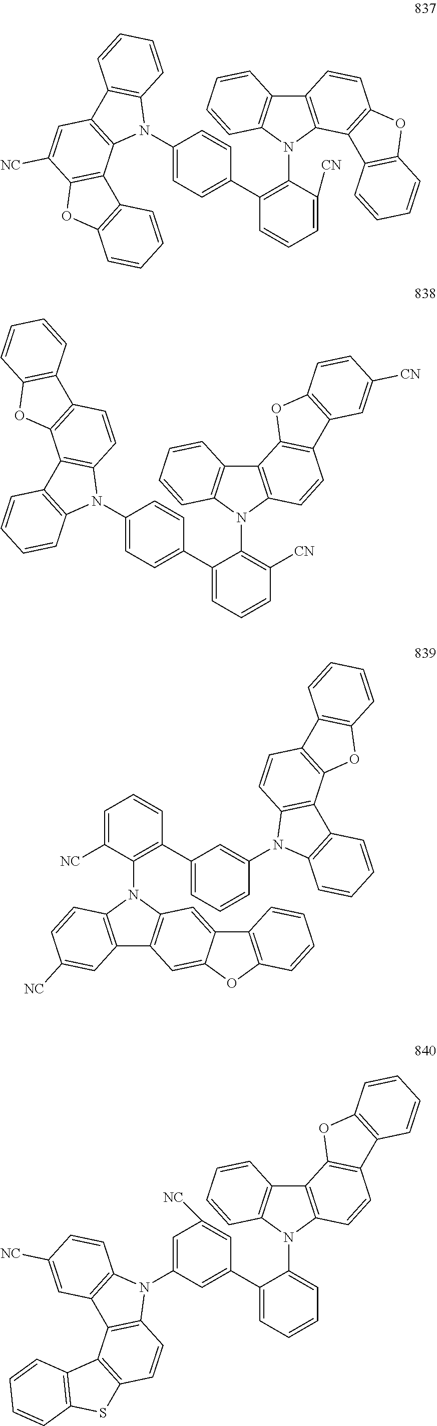 [SMILES: N#Cc1cccc(-c2ccc(-n3c4ccccc4c4cc(C#N)c5oc6ccccc6c5c43)cc2)c1-n1c2ccccc2c2ccc3oc4ccccc4c3c21 ; N#Cc1ccc2oc3c(ccc4c3c3ccccc3n4-c3c(C#N)cccc3-c3ccc(-n4c5ccccc5c5c6oc7ccccc7c6ccc54)cc3)c2c1 ; N#Cc1ccc2c(c1)c1cc3oc4ccccc4c3cc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccc(C#N)cc3c3c4c(ccc32)sc2ccccc24)c1]

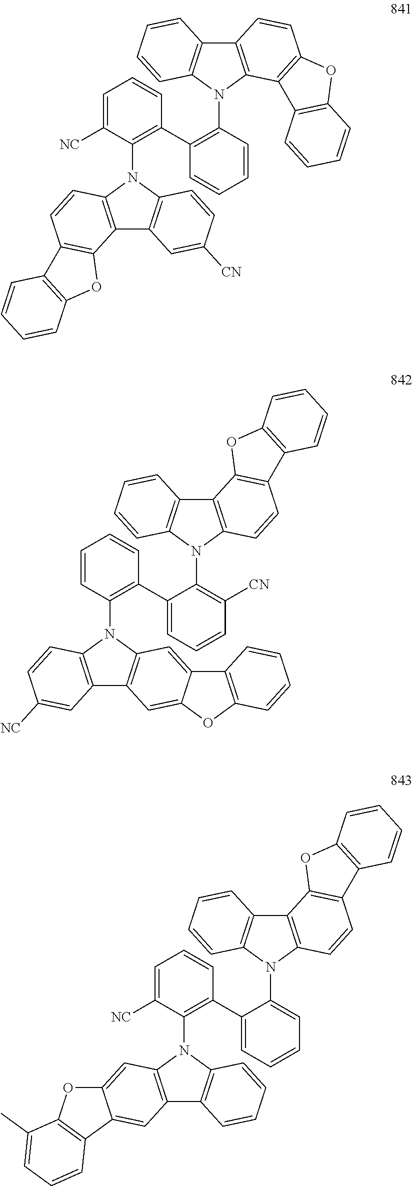 [SMILES: N#Cc1ccc2c(c1)c1c3oc4ccccc4c3ccc1n2-c1c(C#N)cccc1-c1ccccc1-n1c2ccccc2c2ccc3oc4ccccc4c3c21 ; N#Cc1ccc2c(c1)c1cc3oc4ccccc4c3cc1n2-c1ccccc1-c1cccc(C#N)c1-n1c2ccccc2c2c3oc4ccccc4c3ccc21 ; Cc1cccc2c1oc1cc3c(cc12)c1ccccc1n3-c1c(C#N)cccc1-c1ccccc1-n1c2ccccc2c2c3oc4ccccc4c3ccc21]

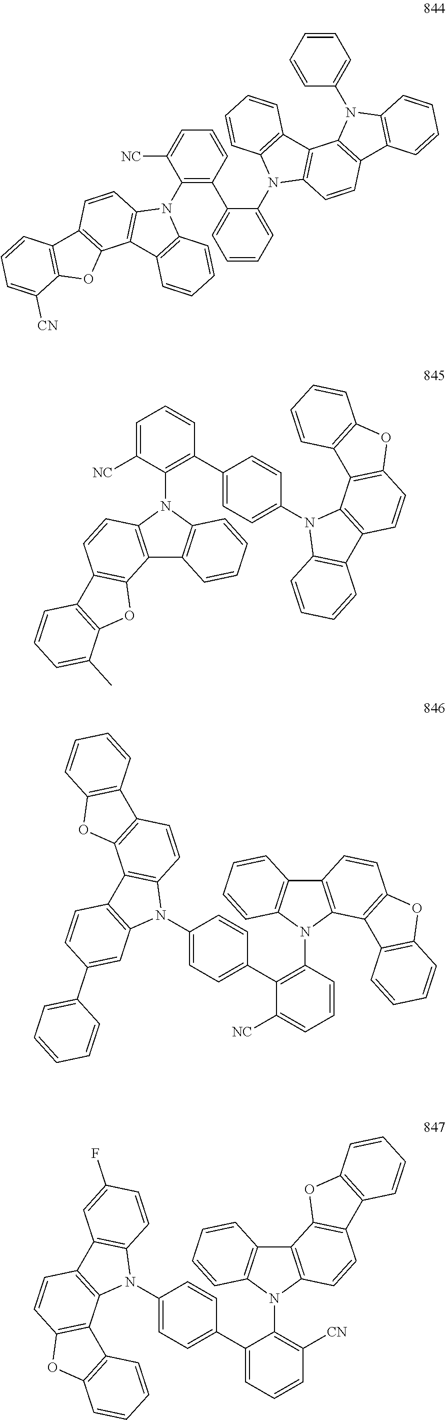 [SMILES: N#Cc1cccc(-c2ccccc2-n2c3ccccc3c3c2ccc2c4ccccc4n(-c4ccccc4)c23)c1-n1c2ccccc2c2c3oc4c(C#N)cccc4c3ccc21 ; Cc1cccc2c1oc1c2ccc2c1c1ccccc1n2-c1c(C#N)cccc1-c1ccc(-n2c3ccccc3c3ccc4oc5ccccc5c4c32)cc1 ; N#Cc1cccc(-n2c3ccccc3c3ccc4oc5ccccc5c4c32)c1-c1ccc(-n2c3cc(-c4ccccc4)ccc3c3c4oc5ccccc5c4ccc32)cc1 ; N#Cc1cccc(-c2ccc(-n3c4ccc(F)cc4c4ccc5oc6ccccc6c5c43)cc2)c1-n1c2ccccc2c2c3oc4ccccc4c3ccc21]

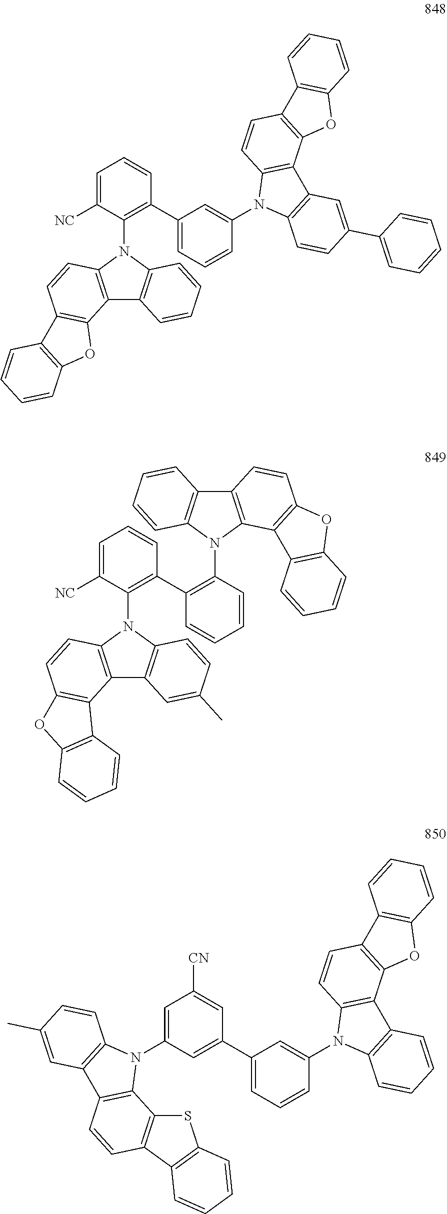 [SMILES: N#Cc1cccc(-c2cccc(-n3c4ccc(-c5ccccc5)cc4c4c5oc6ccccc6c5ccc43)c2)c1-n1c2ccccc2c2c3oc4ccccc4c3ccc21 ; Cc1ccc2c(c1)c1c3c(ccc1n2-c1c(C#N)cccc1-c1ccccc1-n1c2ccccc2c2ccc4oc5ccccc5c4c21)oc1ccccc13 ; Cc1ccc2c(c1)c1ccc3c4ccccc4sc3c1n2-c1cc(C#N)cc(-c2cccc(-n3c4ccccc4c4c5oc6ccccc6c5ccc43)c2)c1]

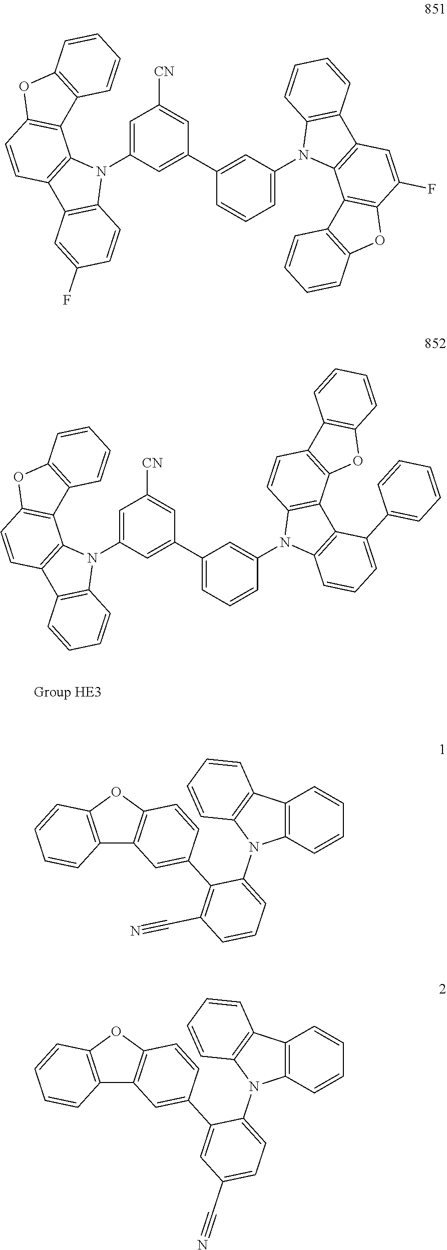 [SMILES: N#Cc1cc(-c2cccc(-n3c4ccccc4c4cc(F)c5oc6ccccc6c5c43)c2)cc(-n2c3ccc(F)cc3c3ccc4oc5ccccc5c4c32)c1 ; N#Cc1cc(-c2cccc(-n3c4cccc(-c5ccccc5)c4c4c5oc6ccccc6c5ccc43)c2)cc(-n2c3ccccc3c3ccc4oc5ccccc5c4c32)c1 ; N#Cc1cccc(-n2c3ccccc3c3ccccc32)c1-c1ccc2oc3ccccc3c2c1 ; N#Cc1ccc(-n2c3ccccc3c3ccccc32)c(-c2ccc3oc4ccccc4c3c2)c1]

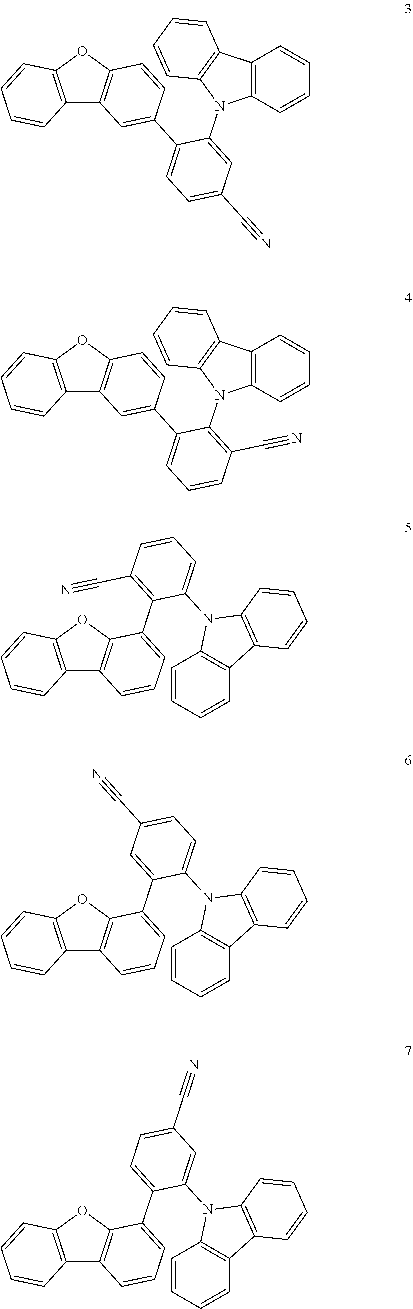 [SMILES: N#Cc1ccc(-c2ccc3oc4ccccc4c3c2)c(-n2c3ccccc3c3ccccc32)c1 ; N#Cc1cccc(-c2ccc3oc4ccccc4c3c2)c1-n1c2ccccc2c2ccccc21 ; N#Cc1cccc(-n2c3ccccc3c3ccccc32)c1-c1cccc2c1oc1ccccc12 ; N#Cc1ccc(-n2c3ccccc3c3ccccc32)c(-c2cccc3c2oc2ccccc23)c1 ; N#Cc1ccc(-c2cccc3c2oc2ccccc23)c(-n2c3ccccc3c3ccccc32)c1]

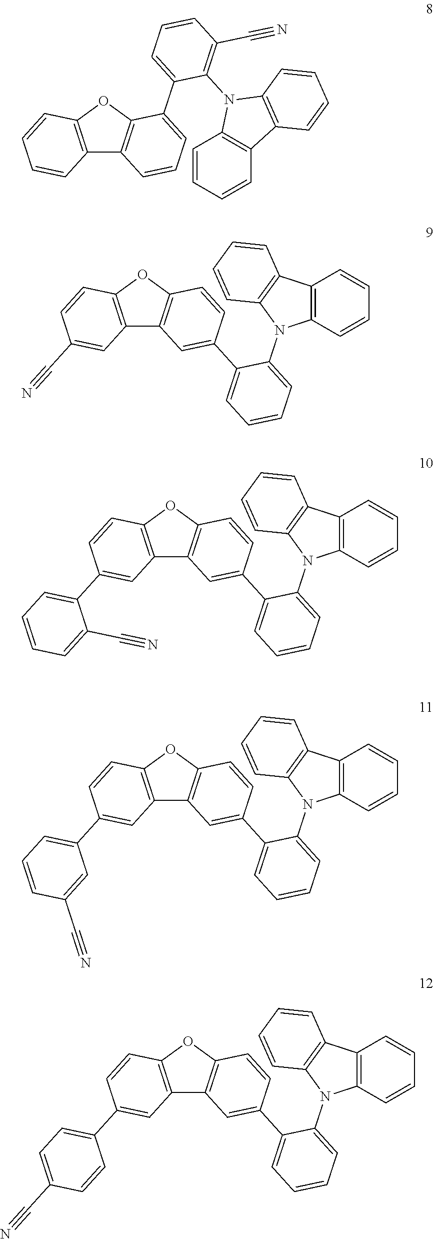

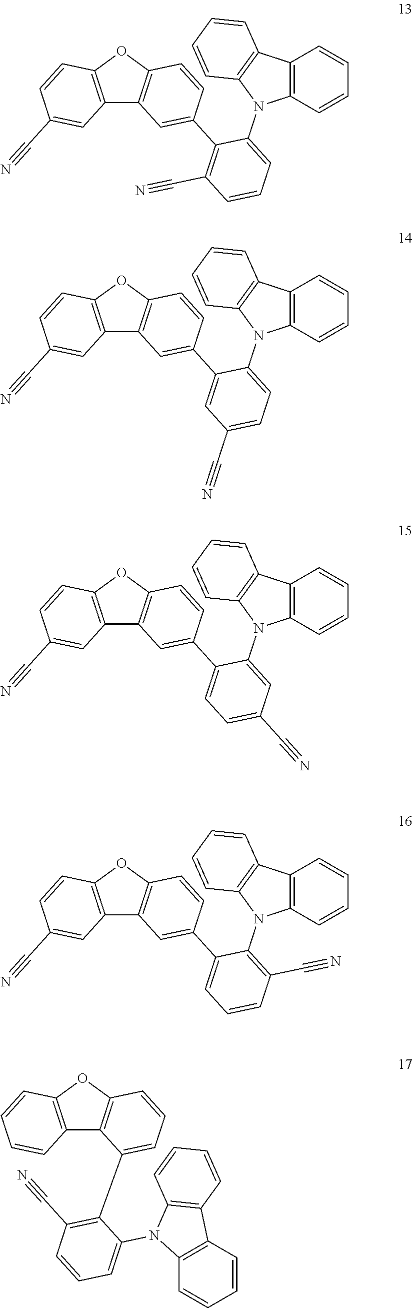

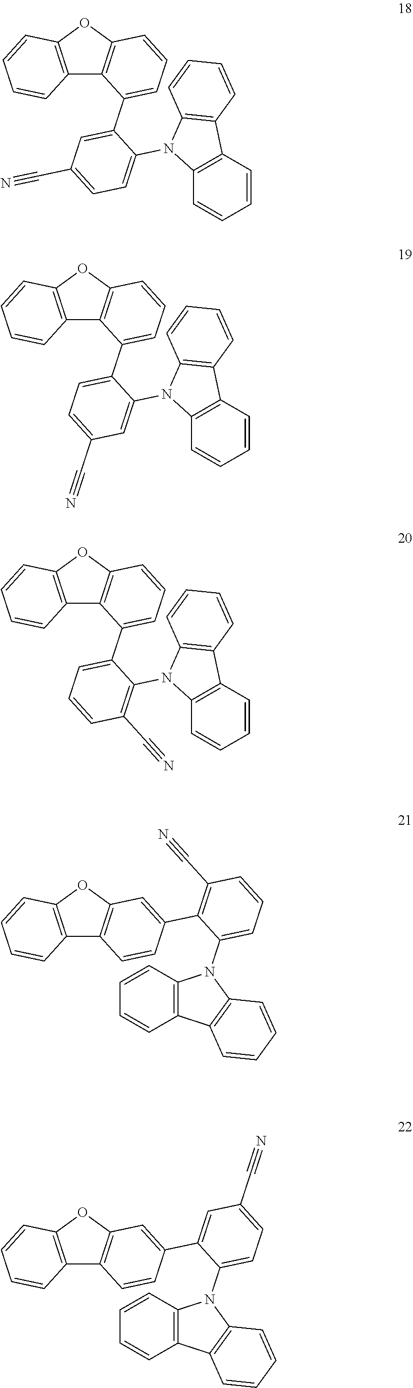 [SMILES: N#Cc1ccc(-n2c3ccccc3c3ccccc32)c(-c2cccc3oc4ccccc4c23)c1 ; N#Cc1ccc(-c2cccc3oc4ccccc4c23)c(-n2c3ccccc3c3ccccc32)c1 ; N#Cc1cccc(-c2cccc3oc4ccccc4c23)c1-n1c2ccccc2c2ccccc21 ; N#Cc1cccc(-n2c3ccccc3c3ccccc32)c1-c1ccc2c(c1)oc1ccccc12 ; N#Cc1ccc(-n2c3ccccc3c3ccccc32)c(-c2ccc3c(c2)oc2ccccc23)c1]

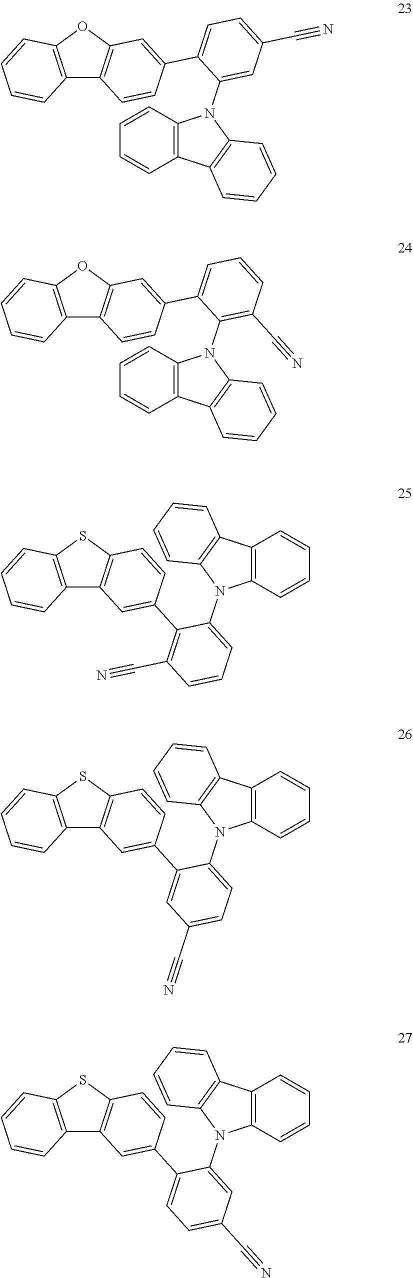 [SMILES: N#Cc1ccc(-c2ccc3c(c2)oc2ccccc23)c(-n2c3ccccc3c3ccccc32)c1 ; N#Cc1cccc(-c2ccc3c(c2)oc2ccccc23)c1-n1c2ccccc2c2ccccc21 ; N#Cc1cccc(-n2c3ccccc3c3ccccc32)c1-c1ccc2sc3ccccc3c2c1 ; N#Cc1ccc(-n2c3ccccc3c3ccccc32)c(-c2ccc3sc4ccccc4c3c2)c1 ; N#Cc1ccc(-c2ccc3sc4ccccc4c3c2)c(-n2c3ccccc3c3ccccc32)c1]

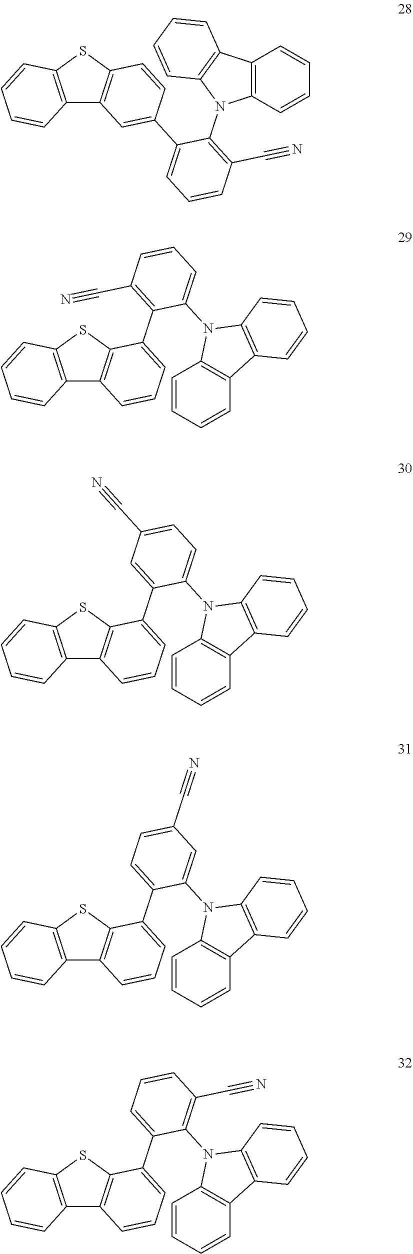 [SMILES: N#Cc1cccc(-c2ccc3sc4ccccc4c3c2)c1-n1c2ccccc2c2ccccc21 ; N#Cc1cccc(-n2c3ccccc3c3ccccc32)c1-c1cccc2c1sc1ccccc12 ; N#Cc1ccc(-n2c3ccccc3c3ccccc32)c(-c2cccc3c2sc2ccccc23)c1 ; N#Cc1ccc(-c2cccc3c2sc2ccccc23)c(-n2c3ccccc3c3ccccc32)c1 ; N#Cc1cccc(-c2cccc3c2sc2ccccc23)c1-n1c2ccccc2c2ccccc21]

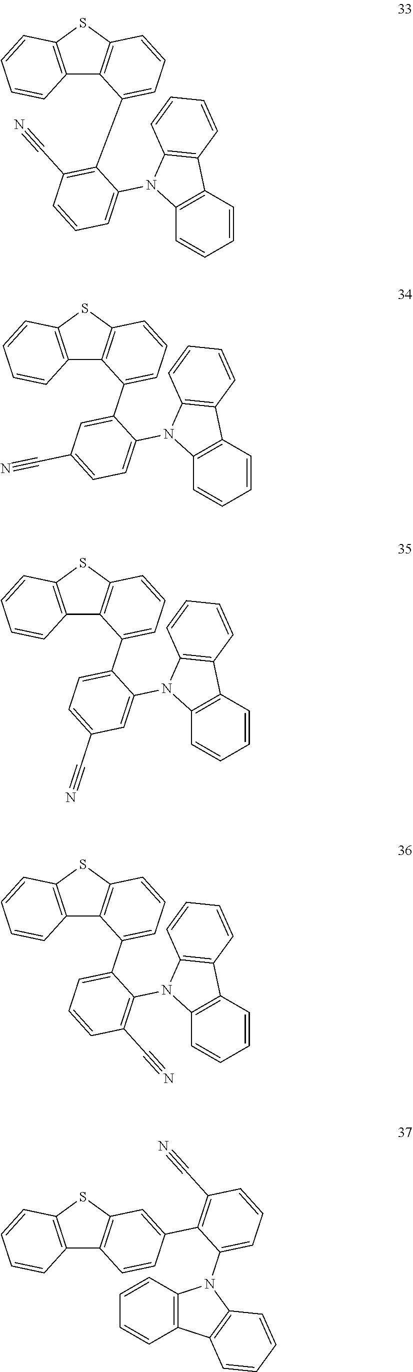 [SMILES: N#Cc1cccc(-n2c3ccccc3c3ccccc32)c1-c1cccc2sc3ccccc3c12 ; N#Cc1ccc(-n2c3ccccc3c3ccccc32)c(-c2cccc3sc4ccccc4c23)c1 ; N#Cc1ccc(-c2cccc3sc4ccccc4c23)c(-n2c3ccccc3c3ccccc32)c1 ; N#Cc1cccc(-c2cccc3sc4ccccc4c23)c1-n1c2ccccc2c2ccccc21 ; N#Cc1cccc(-n2c3ccccc3c3ccccc32)c1-c1ccc2c(c1)sc1ccccc12]

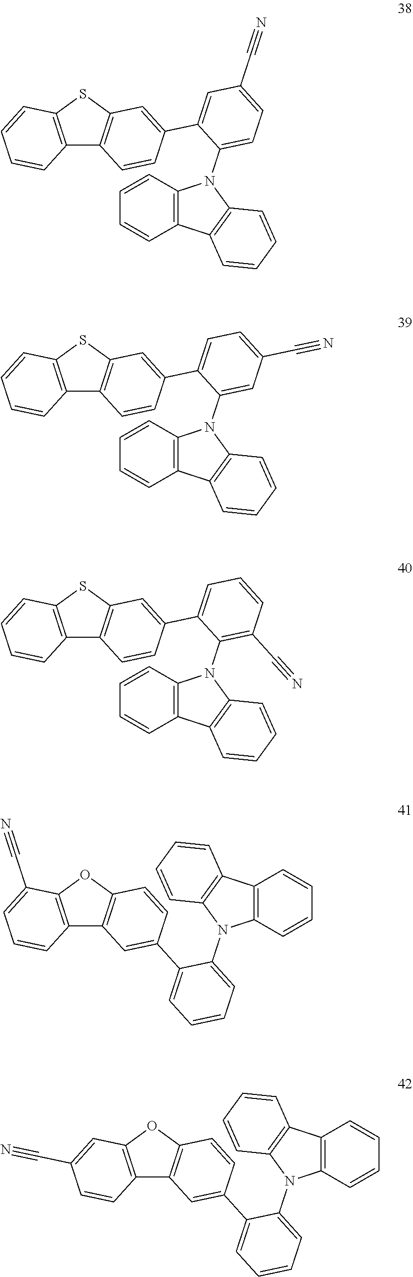 [SMILES: N#Cc1ccc(-n2c3ccccc3c3ccccc32)c(-c2ccc3c(c2)sc2ccccc23)c1 ; N#Cc1ccc(-c2ccc3c(c2)sc2ccccc23)c(-n2c3ccccc3c3ccccc32)c1 ; N#Cc1cccc(-c2ccc3c(c2)sc2ccccc23)c1-n1c2ccccc2c2ccccc21 ; N#Cc1cccc2c1oc1ccc(-c3ccccc3-n3c4ccccc4c4ccccc43)cc12 ; N#Cc1ccc2c(c1)oc1ccc(-c3ccccc3-n3c4ccccc4c4ccccc43)cc12]

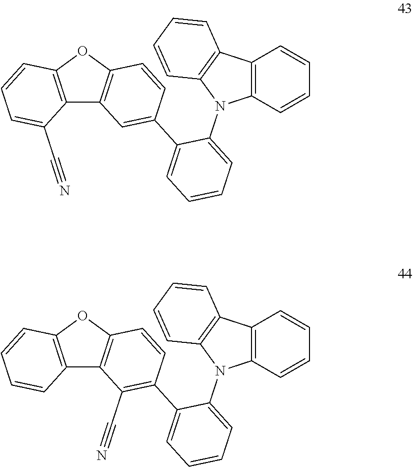 [SMILES: N#Cc1cccc2oc3ccc(-c4ccccc4-n4c5ccccc5c5ccccc54)cc3c12 ; N#Cc1c(-c2ccccc2-n2c3ccccc3c3ccccc32)ccc2oc3ccccc3c12]

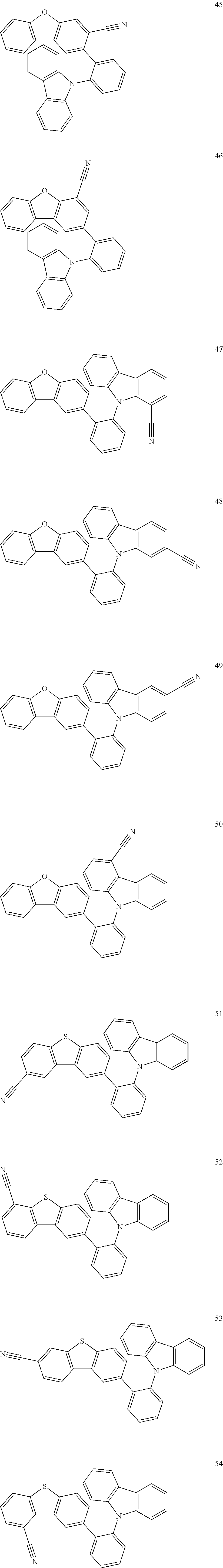

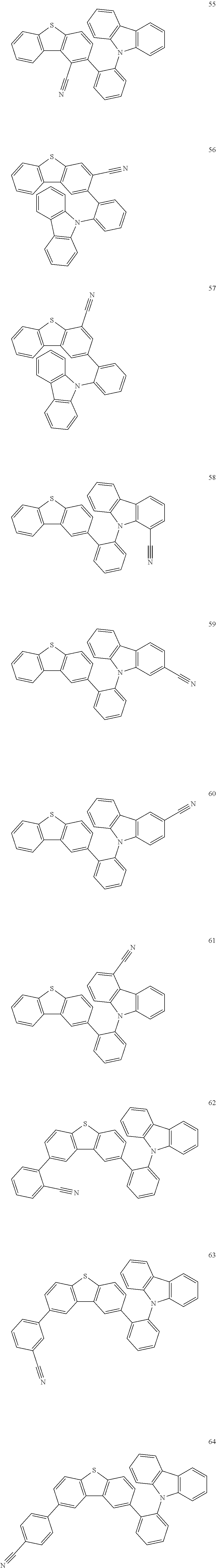

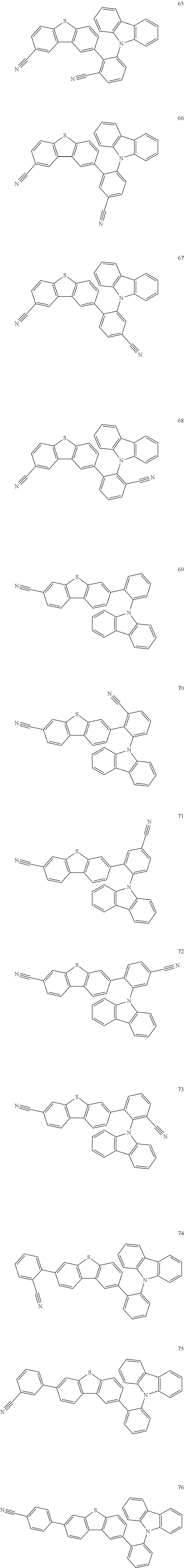

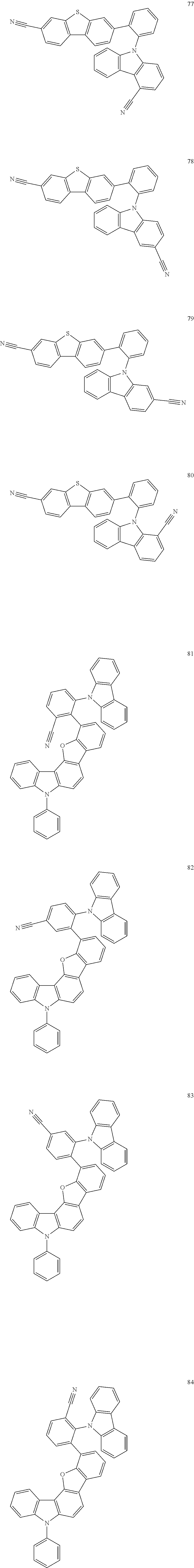

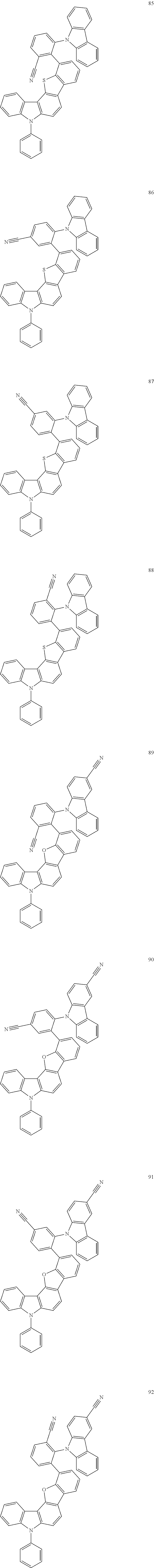

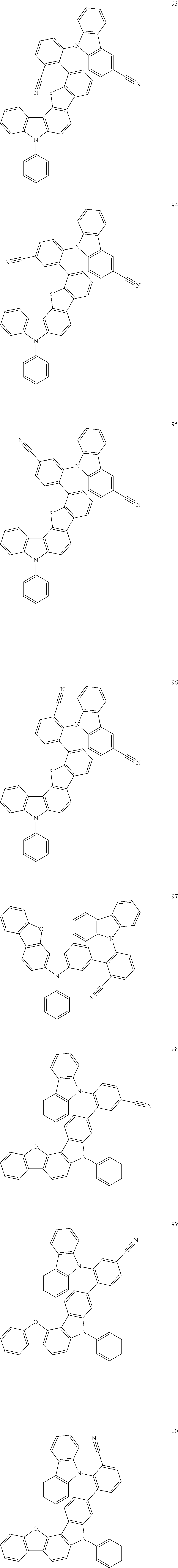

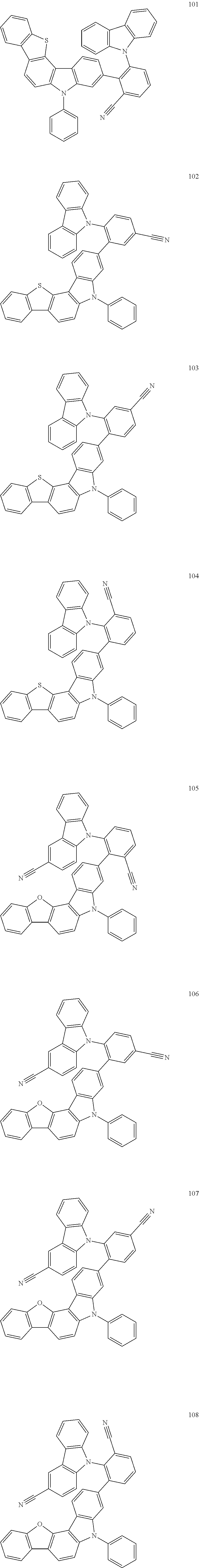

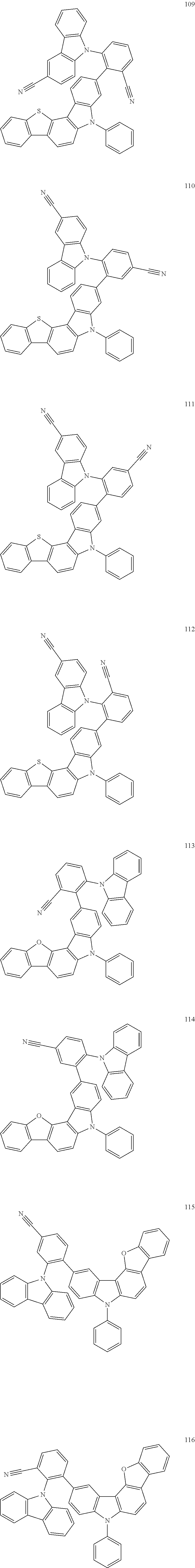

[chemical structure]

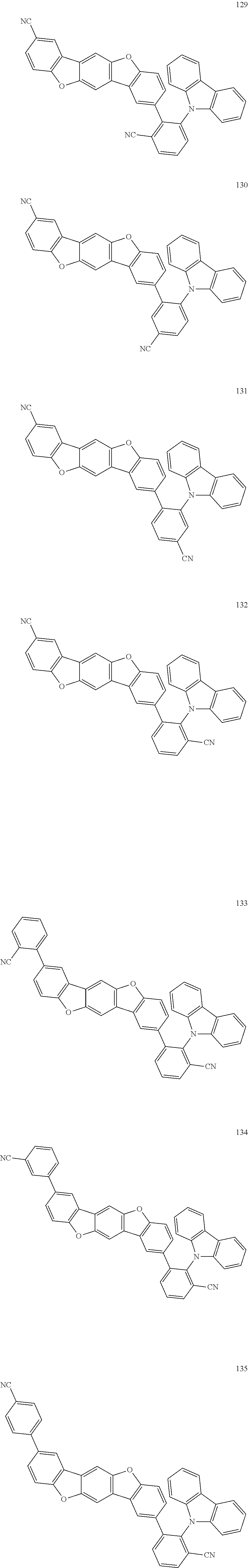

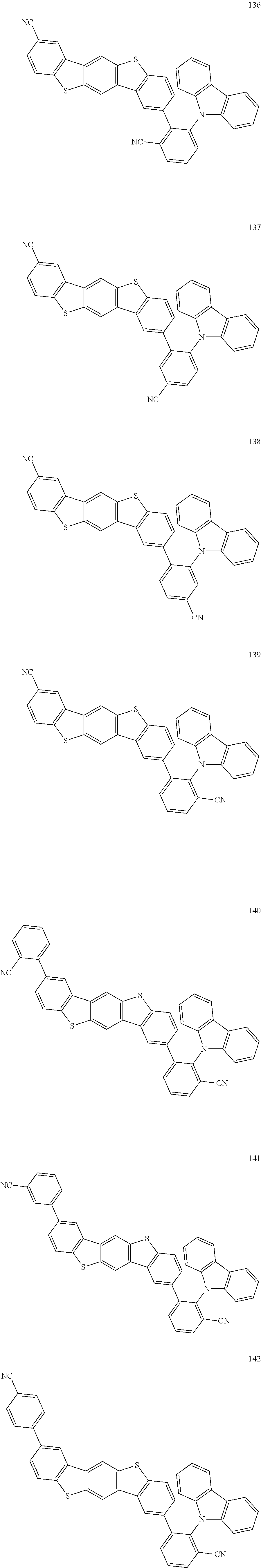

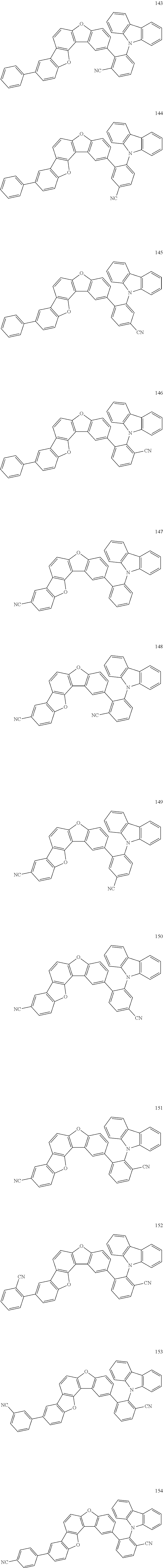

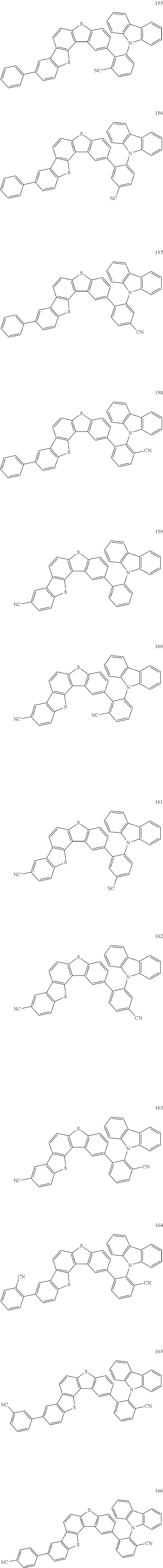

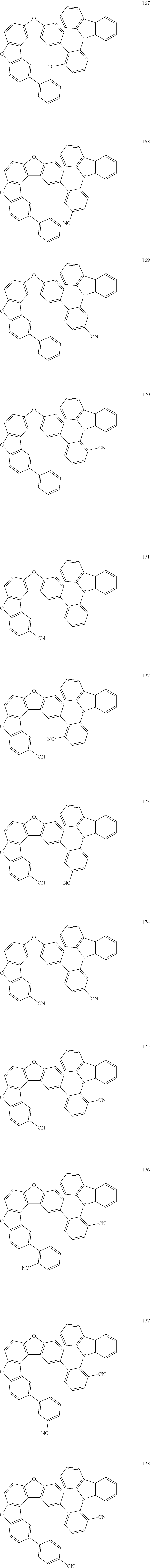

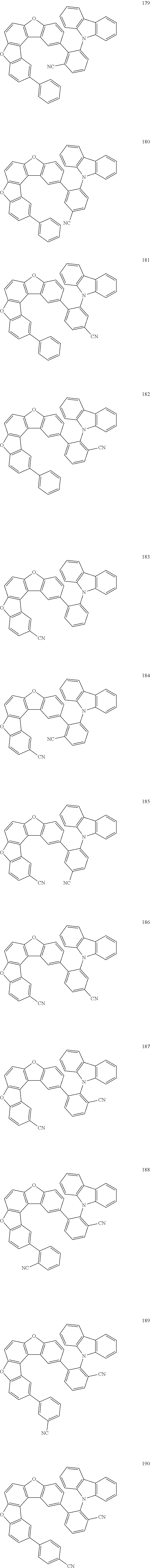

[chemical structure]

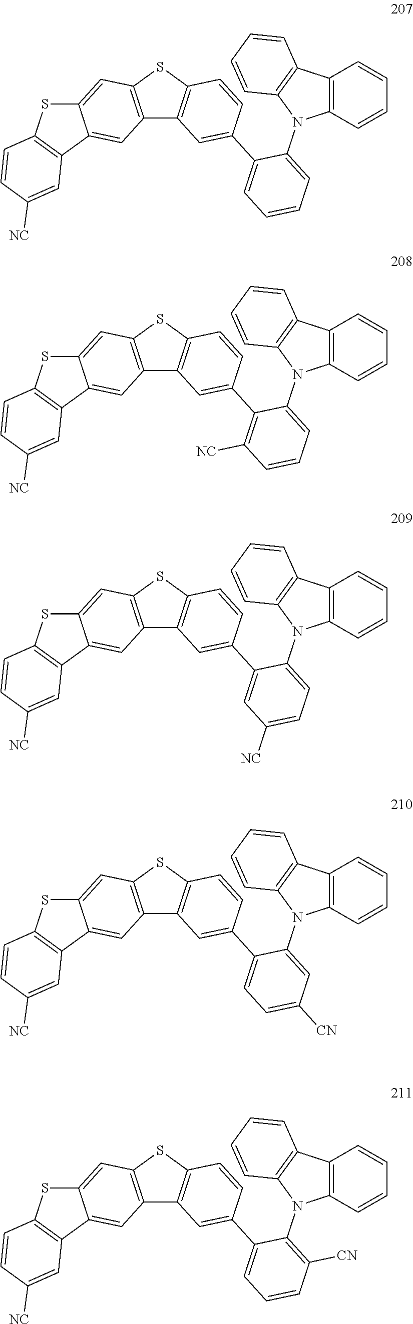 [SMILES: N#Cc1ccc2sc3cc4sc5ccc(-c6ccccc6-n6c7ccccc7c7ccccc76)cc5c4cc3c2c1 ; N#Cc1ccc2sc3cc4sc5ccc(-c6c(C#N)cccc6-n6c7ccccc7c7ccccc76)cc5c4cc3c2c1 ; N#Cc1ccc(-n2c3ccccc3c3ccccc32)c(-c2ccc3sc4cc5sc6ccc(C#N)cc6c5cc4c3c2)c1 ; N#Cc1ccc(-c2ccc3sc4cc5sc6ccc(C#N)cc6c5cc4c3c2)c(-n2c3ccccc3c3ccccc32)c1 ; N#Cc1ccc2sc3cc4sc5ccc(-c6cccc(C#N)c6-n6c7ccccc7c7ccccc76)cc5c4cc3c2c1]

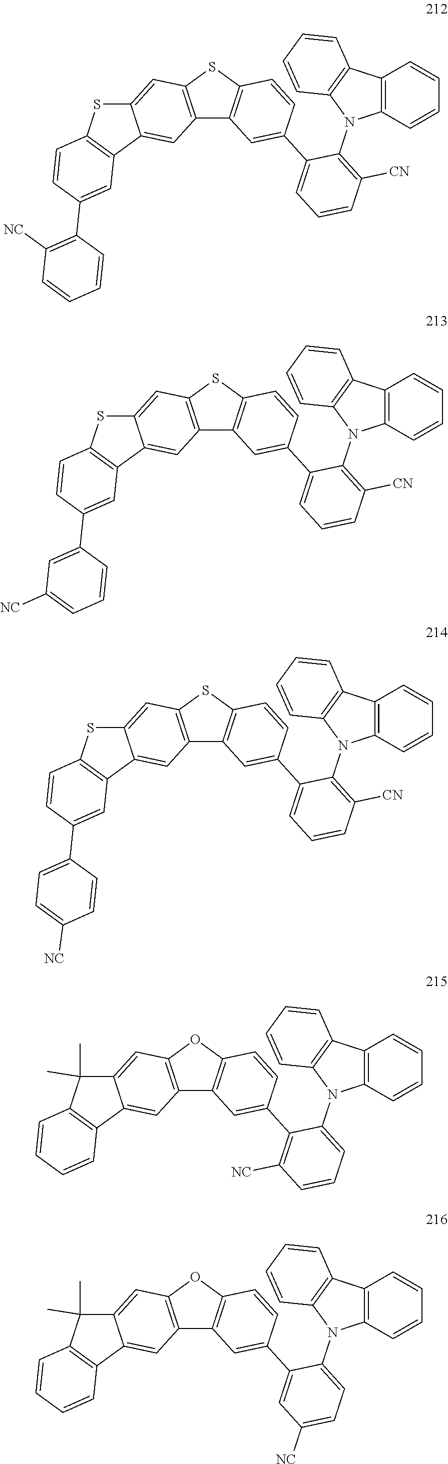 [SMILES: N#Cc1ccccc1-c1ccc2sc3cc4sc5ccc(-c6cccc(C#N)c6-n6c7ccccc7c7ccccc76)cc5c4cc3c2c1 ; N#Cc1cccc(-c2ccc3sc4cc5sc6ccc(-c7cccc(C#N)c7-n7c8ccccc8c8ccccc87)cc6c5cc4c3c2)c1 ; N#Cc1ccc(-c2ccc3sc4cc5sc6ccc(-c7cccc(C#N)c7-n7c8ccccc8c8ccccc87)cc6c5cc4c3c2)cc1 ; CC1(C)c2ccccc2-c2cc3c(cc21)oc1ccc(-c2c(C#N)cccc2-n2c4ccccc4c4ccccc42)cc13 ; CC1(C)c2ccccc2-c2cc3c(cc21)oc1ccc(-c2cc(C#N)ccc2-n2c4ccccc4c4ccccc42)cc13]

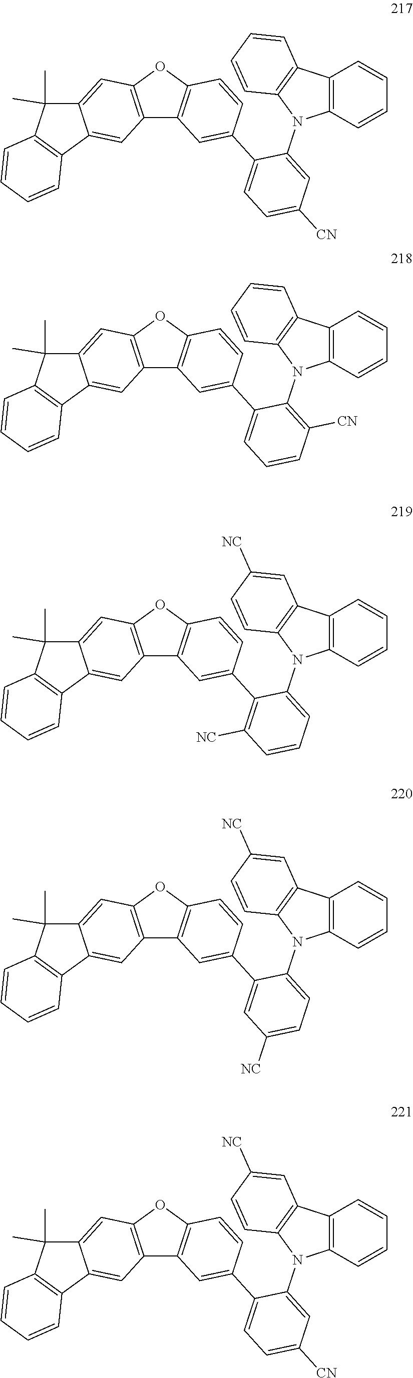 [SMILES: CC1(C)c2ccccc2-c2cc3c(cc21)oc1ccc(-c2ccc(C#N)cc2-n2c4ccccc4c4ccccc42)cc13 ; CC1(C)c2ccccc2-c2cc3c(cc21)oc1ccc(-c2cccc(C#N)c2-n2c4ccccc4c4ccccc42)cc13 ; CC1(C)c2ccccc2-c2cc3c(cc21)oc1ccc(-c2c(C#N)cccc2-n2c4ccccc4c4cc(C#N)ccc42)cc13 ; CC1(C)c2ccccc2-c2cc3c(cc21)oc1ccc(-c2cc(C#N)ccc2-n2c4ccccc4c4cc(C#N)ccc42)cc13 ; CC1(C)c2ccccc2-c2cc3c(cc21)oc1ccc(-c2ccc(C#N)cc2-n2c4ccccc4c4cc(C#N)ccc42)cc13]

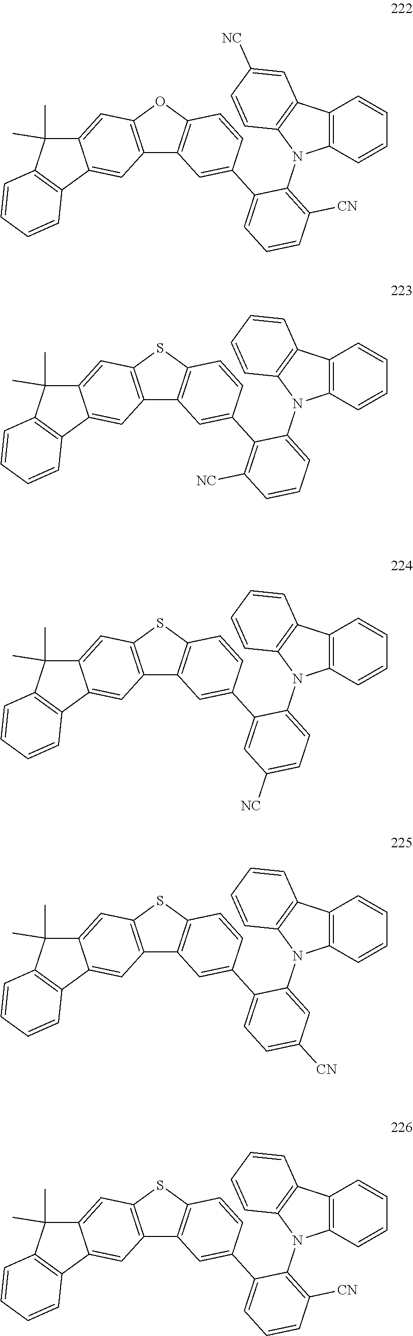 [SMILES: CC1(C)c2ccccc2-c2cc3c(cc21)oc1ccc(-c2cccc(C#N)c2-n2c4ccccc4c4cc(C#N)ccc42)cc13 ; CC1(C)c2ccccc2-c2cc3c(cc21)sc1ccc(-c2c(C#N)cccc2-n2c4ccccc4c4ccccc42)cc13 ; CC1(C)c2ccccc2-c2cc3c(cc21)sc1ccc(-c2cc(C#N)ccc2-n2c4ccccc4c4ccccc42)cc13 ; CC1(C)c2ccccc2-c2cc3c(cc21)sc1ccc(-c2ccc(C#N)cc2-n2c4ccccc4c4ccccc42)cc13 ; CC1(C)c2ccccc2-c2cc3c(cc21)sc1ccc(-c2cccc(C#N)c2-n2c4ccccc4c4ccccc42)cc13]

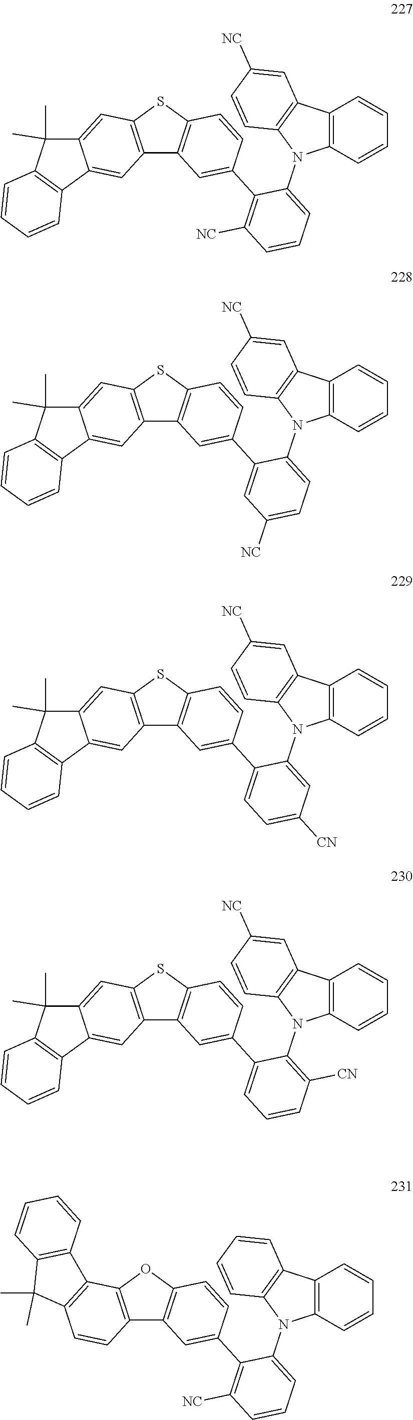 [SMILES: CC1(C)c2ccccc2-c2cc3c(cc21)sc1ccc(-c2c(C#N)cccc2-n2c4ccccc4c4cc(C#N)ccc42)cc13 ; CC1(C)c2ccccc2-c2cc3c(cc21)sc1ccc(-c2cc(C#N)ccc2-n2c4ccccc4c4cc(C#N)ccc42)cc13 ; CC1(C)c2ccccc2-c2cc3c(cc21)sc1ccc(-c2ccc(C#N)cc2-n2c4ccccc4c4cc(C#N)ccc42)cc13 ; CC1(C)c2ccccc2-c2cc3c(cc21)sc1ccc(-c2cccc(C#N)c2-n2c4ccccc4c4cc(C#N)ccc42)cc13 ; CC1(C)c2ccccc2-c2c1ccc1c2oc2ccc(-c3c(C#N)cccc3-n3c4ccccc4c4ccccc43)cc21]

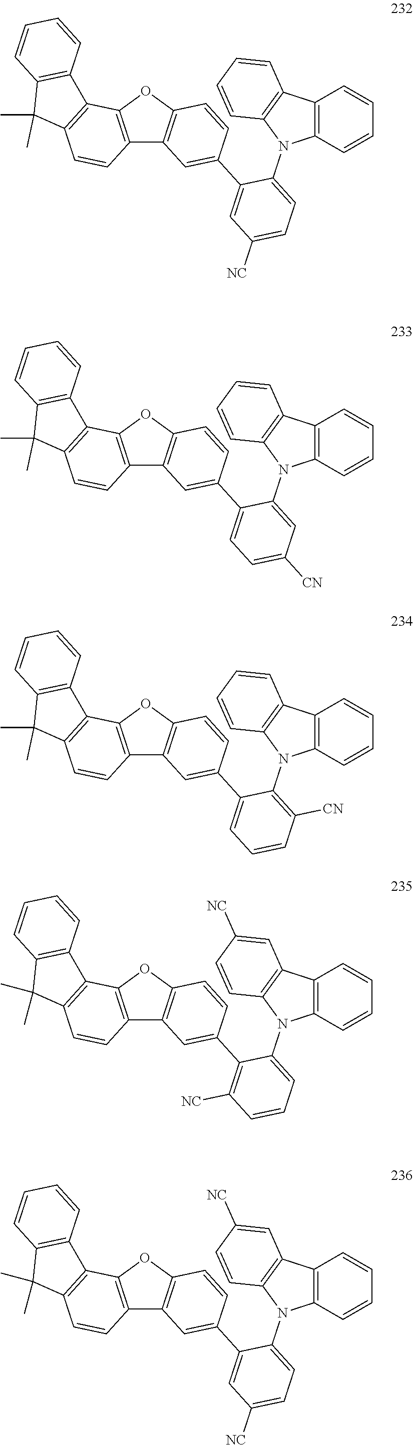 [SMILES: CC1(C)c2ccccc2-c2c1ccc1c2oc2ccc(-c3cc(C#N)ccc3-n3c4ccccc4c4ccccc43)cc21 ; CC1(C)c2ccccc2-c2c1ccc1c2oc2ccc(-c3ccc(C#N)cc3-n3c4ccccc4c4ccccc43)cc21 ; CC1(C)c2ccccc2-c2c1ccc1c2oc2ccc(-c3cccc(C#N)c3-n3c4ccccc4c4ccccc43)cc21 ; CC1(C)c2ccccc2-c2c1ccc1c2oc2ccc(-c3c(C#N)cccc3-n3c4ccccc4c4cc(C#N)ccc43)cc21 ; CC1(C)c2ccccc2-c2c1ccc1c2oc2ccc(-c3cc(C#N)ccc3-n3c4ccccc4c4cc(C#N)ccc43)cc21]

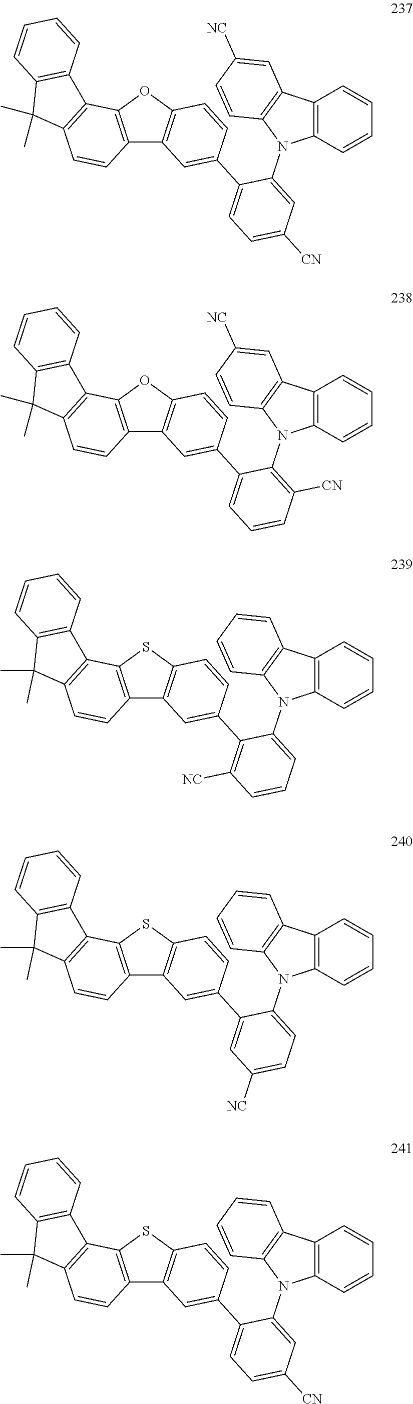 [SMILES: CC1(C)c2ccccc2-c2c1ccc1c2oc2ccc(-c3ccc(C#N)cc3-n3c4ccccc4c4cc(C#N)ccc43)cc21 ; CC1(C)c2ccccc2-c2c1ccc1c2oc2ccc(-c3cccc(C#N)c3-n3c4ccccc4c4cc(C#N)ccc43)cc21 ; CC1(C)c2ccccc2-c2c1ccc1c2sc2ccc(-c3c(C#N)cccc3-n3c4ccccc4c4ccccc43)cc21 ; CC1(C)c2ccccc2-c2c1ccc1c2sc2ccc(-c3cc(C#N)ccc3-n3c4ccccc4c4ccccc43)cc21 ; CC1(C)c2ccccc2-c2c1ccc1c2sc2ccc(-c3ccc(C#N)cc3-n3c4ccccc4c4ccccc43)cc21]

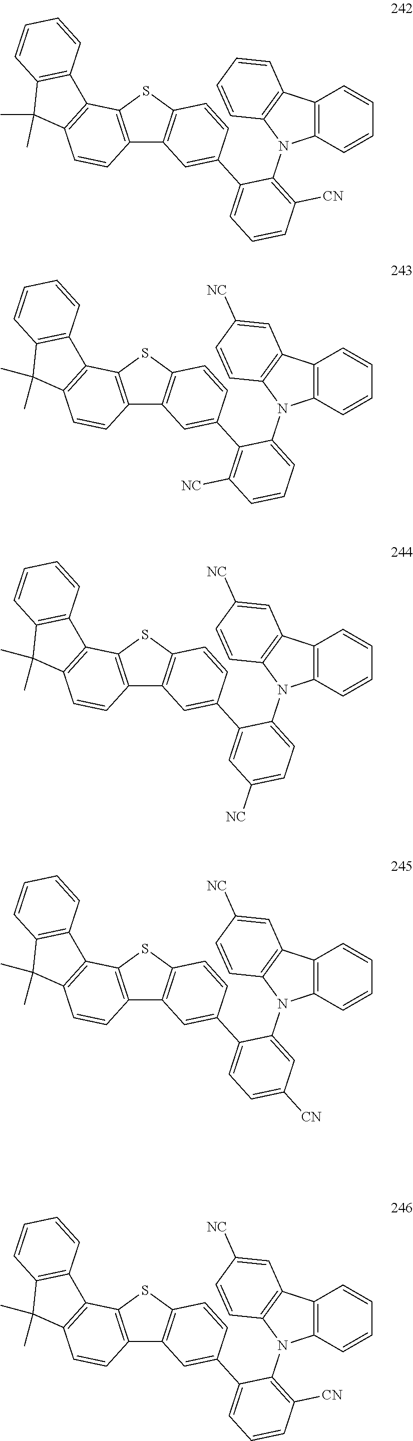 [SMILES: CC1(C)c2ccccc2-c2c1ccc1c2sc2ccc(-c3cccc(C#N)c3-n3c4ccccc4c4ccccc43)cc21 ; CC1(C)c2ccccc2-c2c1ccc1c2sc2ccc(-c3c(C#N)cccc3-n3c4ccccc4c4cc(C#N)ccc43)cc21 ; CC1(C)c2ccccc2-c2c1ccc1c2sc2ccc(-c3cc(C#N)ccc3-n3c4ccccc4c4cc(C#N)ccc43)cc21 ; CC1(C)c2ccccc2-c2c1ccc1c2sc2ccc(-c3ccc(C#N)cc3-n3c4ccccc4c4cc(C#N)ccc43)cc21 ; CC1(C)c2ccccc2-c2c1ccc1c2sc2ccc(-c3cccc(C#N)c3-n3c4ccccc4c4cc(C#N)ccc43)cc21]

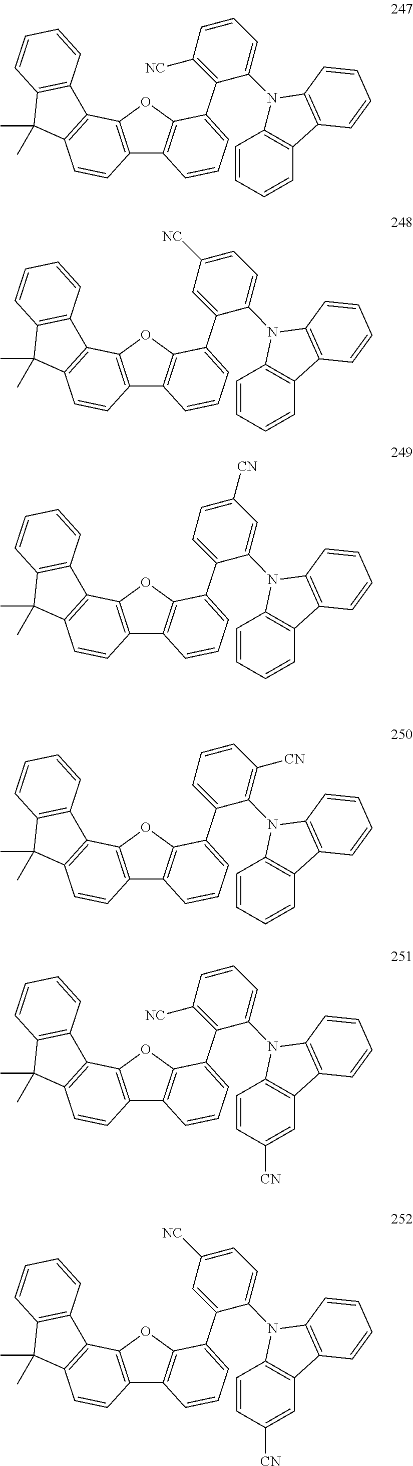

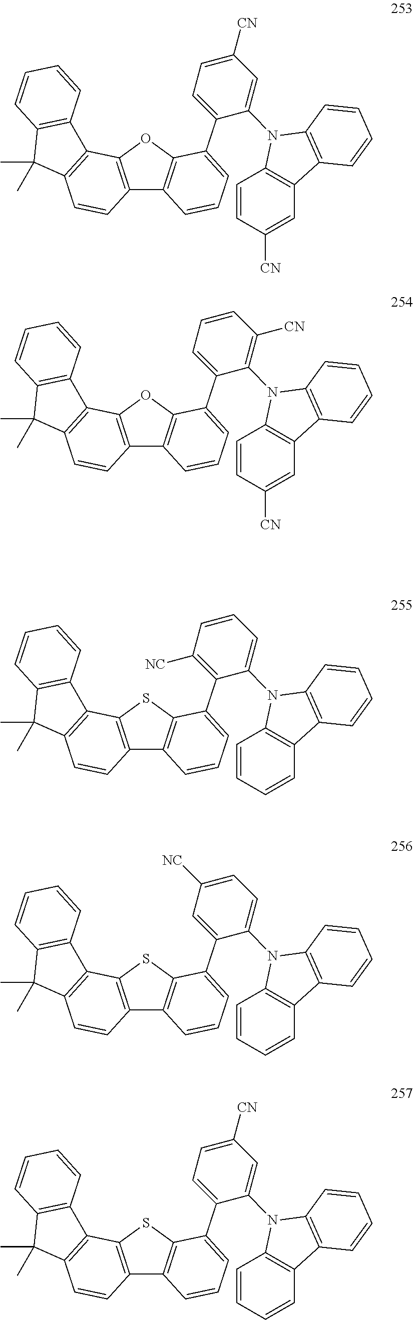 [SMILES: CC1(C)c2ccccc2-c2c1ccc1c2oc2c(-c3ccc(C#N)cc3-n3c4ccccc4c4cc(C#N)ccc43)cccc21 ; CC1(C)c2ccccc2-c2c1ccc1c2oc2c(-c3cccc(C#N)c3-n3c4ccccc4c4cc(C#N)ccc43)cccc21 ; CC1(C)c2ccccc2-c2c1ccc1c2sc2c(-c3c(C#N)cccc3-n3c4ccccc4c4ccccc43)cccc21 ; CC1(C)c2ccccc2-c2c1ccc1c2sc2c(-c3cc(C#N)ccc3-n3c4ccccc4c4ccccc43)cccc21 ; CC1(C)c2ccccc2-c2c1ccc1c2sc2c(-c3ccc(C#N)cc3-n3c4ccccc4c4ccccc43)cccc21]

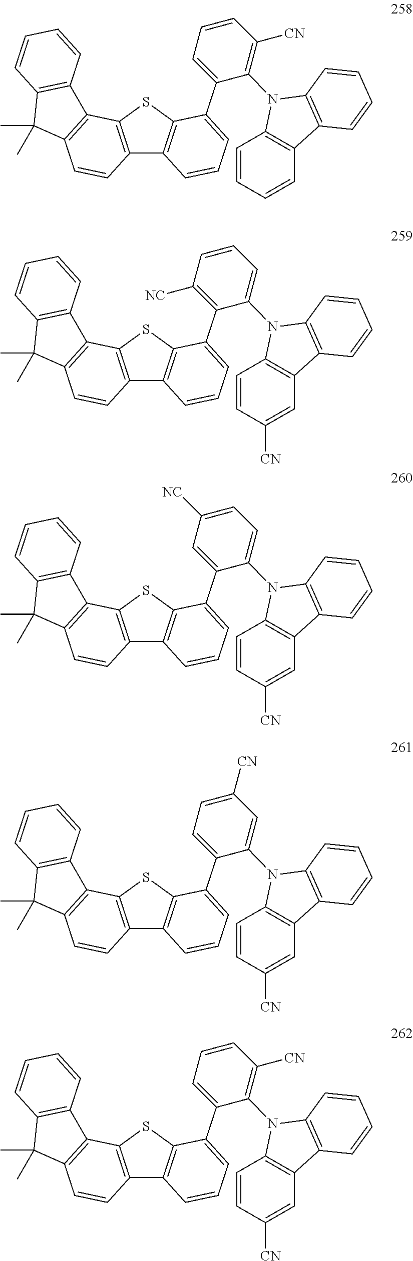 [SMILES: CC1(C)c2ccccc2-c2c1ccc1c2sc2c(-c3cccc(C#N)c3-n3c4ccccc4c4ccccc43)cccc21 ; CC1(C)c2ccccc2-c2c1ccc1c2sc2c(-c3c(C#N)cccc3-n3c4ccccc4c4cc(C#N)ccc43)cccc21 ; CC1(C)c2ccccc2-c2c1ccc1c2sc2c(-c3cc(C#N)ccc3-n3c4ccccc4c4cc(C#N)ccc43)cccc21 ; CC1(C)c2ccccc2-c2c1ccc1c2sc2c(-c3ccc(C#N)cc3-n3c4ccccc4c4cc(C#N)ccc43)cccc21 ; CC1(C)c2ccccc2-c2c1ccc1c2sc2c(-c3cccc(C#N)c3-n3c4ccccc4c4cc(C#N)ccc43)cccc21]

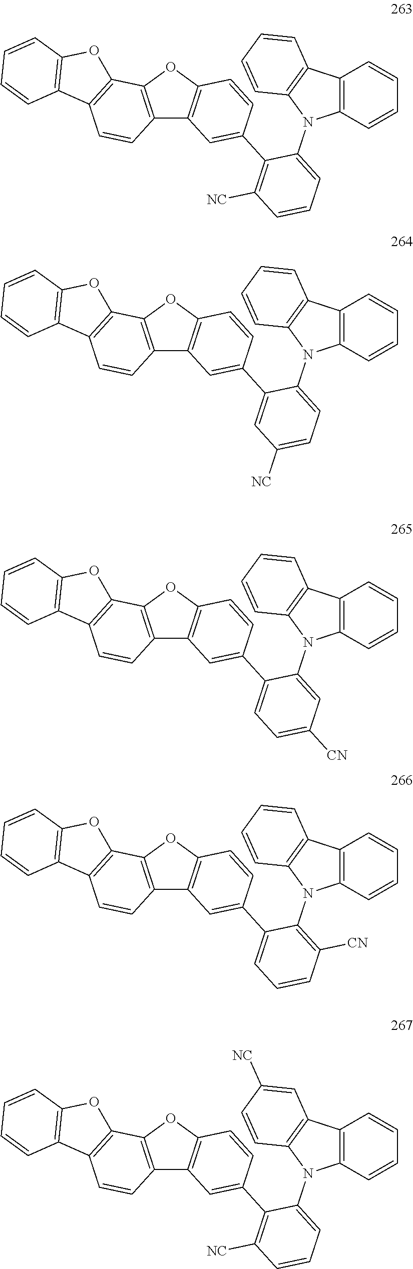 [SMILES: N#Cc1cccc(-n2c3ccccc3c3ccccc32)c1-c1ccc2oc3c(ccc4c5ccccc5oc43)c2c1 ; N#Cc1ccc(-n2c3ccccc3c3ccccc32)c(-c2ccc3oc4c(ccc5c6ccccc6oc54)c3c2)c1 ; N#Cc1ccc(-c2ccc3oc4c(ccc5c6ccccc6oc54)c3c2)c(-n2c3ccccc3c3ccccc32)c1 ; N#Cc1cccc(-c2ccc3oc4c(ccc5c6ccccc6oc54)c3c2)c1-n1c2ccccc2c2ccccc21 ; N#Cc1ccc2c(c1)c1ccccc1n2-c1cccc(C#N)c1-c1ccc2oc3c(ccc4c5ccccc5oc43)c2c1]

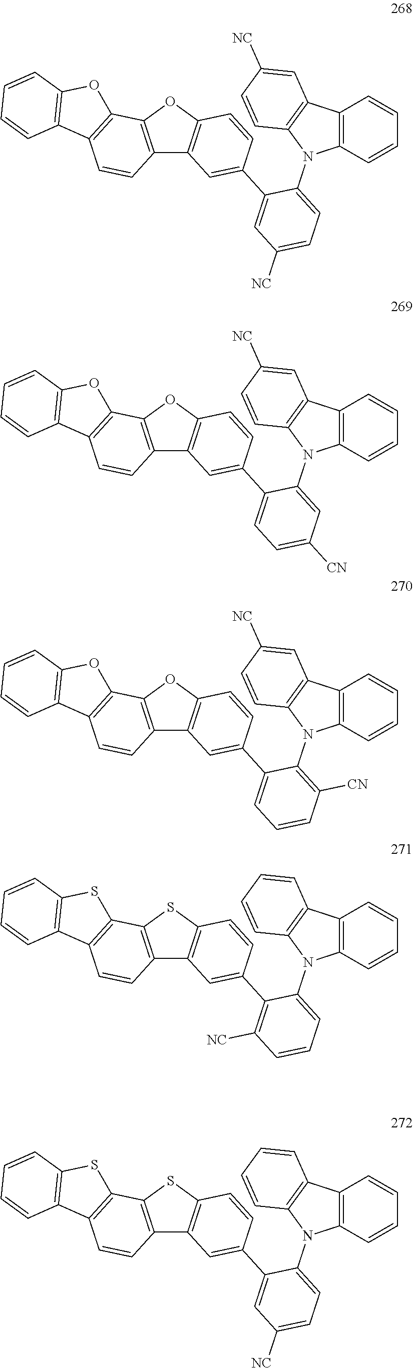 [SMILES: N#Cc1ccc(-n2c3ccccc3c3cc(C#N)ccc32)c(-c2ccc3oc4c(ccc5c6ccccc6oc54)c3c2)c1 ; N#Cc1ccc(-c2ccc3oc4c(ccc5c6ccccc6oc54)c3c2)c(-n2c3ccccc3c3cc(C#N)ccc32)c1 ; N#Cc1ccc2c(c1)c1ccccc1n2-c1c(C#N)cccc1-c1ccc2oc3c(ccc4c5ccccc5oc43)c2c1 ; N#Cc1cccc(-n2c3ccccc3c3ccccc32)c1-c1ccc2sc3c(ccc4c5ccccc5sc43)c2c1 ; N#Cc1ccc(-n2c3ccccc3c3ccccc32)c(-c2ccc3sc4c(ccc5c6ccccc6sc54)c3c2)c1]

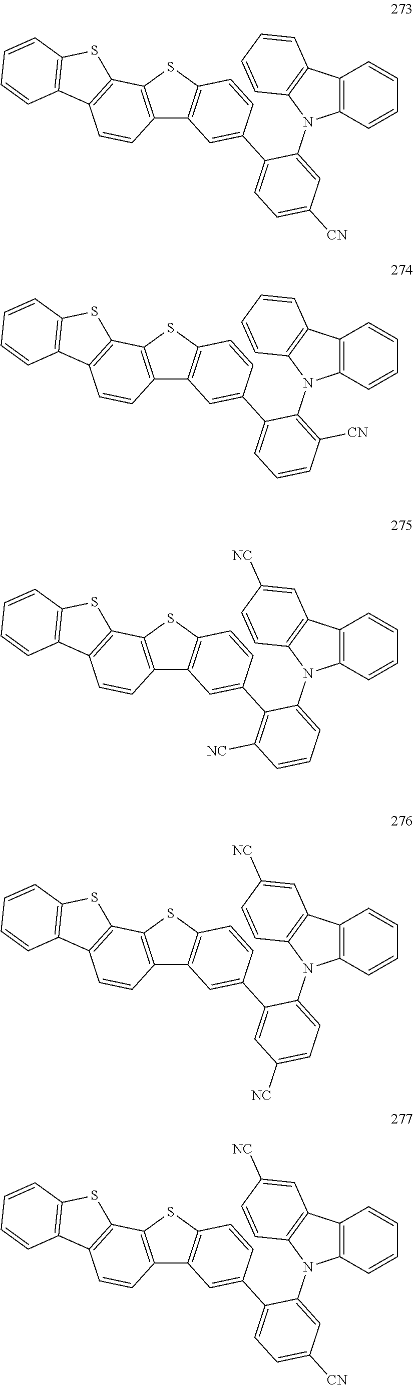 [SMILES: N#Cc1ccc(-c2ccc3sc4c(ccc5c6ccccc6sc54)c3c2)c(-n2c3ccccc3c3ccccc32)c1 ; N#Cc1cccc(-c2ccc3sc4c(ccc5c6ccccc6sc54)c3c2)c1-n1c2ccccc2c2ccccc21 ; N#Cc1ccc2c(c1)c1ccccc1n2-c1cccc(C#N)c1-c1ccc2sc3c(ccc4c5ccccc5sc43)c2c1 ; N#Cc1ccc(-n2c3ccccc3c3cc(C#N)ccc32)c(-c2ccc3sc4c(ccc5c6ccccc6sc54)c3c2)c1 ; N#Cc1ccc(-c2ccc3sc4c(ccc5c6ccccc6sc54)c3c2)c(-n2c3ccccc3c3cc(C#N)ccc32)c1]

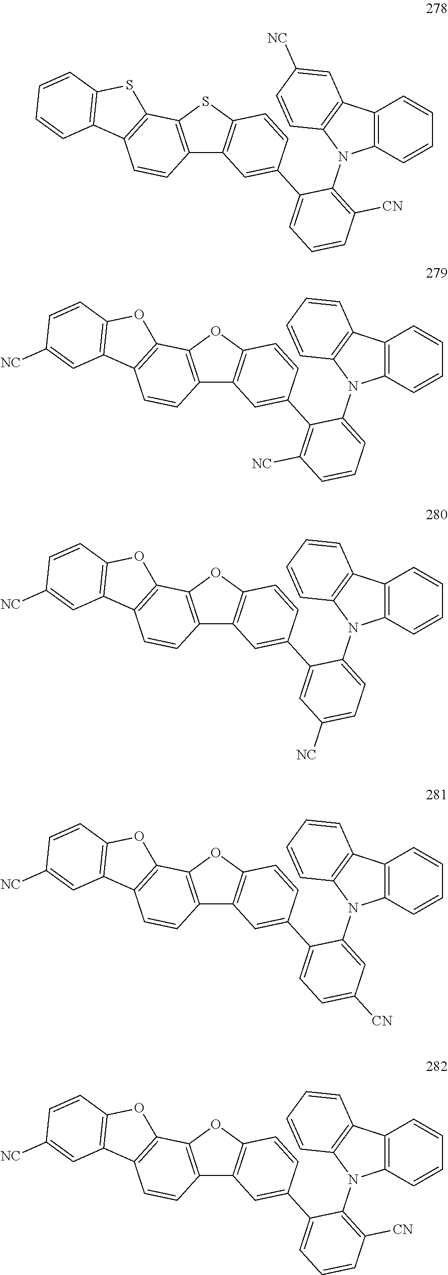 [SMILES: N#Cc1ccc2c(c1)c1ccccc1n2-c1c(C#N)cccc1-c1ccc2sc3c(ccc4c5ccccc5sc43)c2c1 ; N#Cc1ccc2oc3c(ccc4c5cc(-c6c(C#N)cccc6-n6c7ccccc7c7ccccc76)ccc5oc43)c2c1 ; N#Cc1ccc(-n2c3ccccc3c3ccccc32)c(-c2ccc3oc4c(ccc5c6cc(C#N)ccc6oc54)c3c2)c1 ; N#Cc1ccc(-c2ccc3oc4c(ccc5c6cc(C#N)ccc6oc54)c3c2)c(-n2c3ccccc3c3ccccc32)c1 ; N#Cc1ccc2oc3c(ccc4c5cc(-c6cccc(C#N)c6-n6c7ccccc7c7ccccc76)ccc5oc43)c2c1]

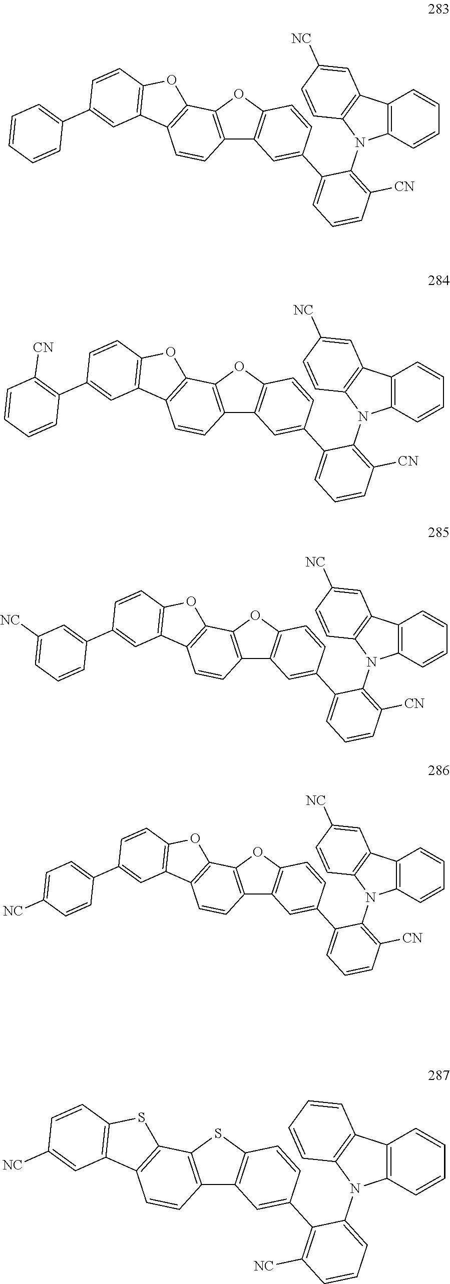 [SMILES: N#Cc1ccc2c(c1)c1ccccc1n2-c1c(C#N)cccc1-c1ccc2oc3c(ccc4c5cc(-c6ccccc6)ccc5oc43)c2c1 ; N#Cc1ccc2c(c1)c1ccccc1n2-c1c(C#N)cccc1-c1ccc2oc3c(ccc4c5cc(-c6ccccc6C#N)ccc5oc43)c2c1 ; N#Cc1cccc(-c2ccc3oc4c(ccc5c6cc(-c7cccc(C#N)c7-n7c8ccccc8c8cc(C#N)ccc87)ccc6oc54)c3c2)c1 ; N#Cc1ccc(-c2ccc3oc4c(ccc5c6cc(-c7cccc(C#N)c7-n7c8ccccc8c8cc(C#N)ccc87)ccc6oc54)c3c2)cc1 ; N#Cc1ccc2sc3c(ccc4c5cc(-c6c(C#N)cccc6-n6c7ccccc7c7ccccc76)ccc5sc43)c2c1]

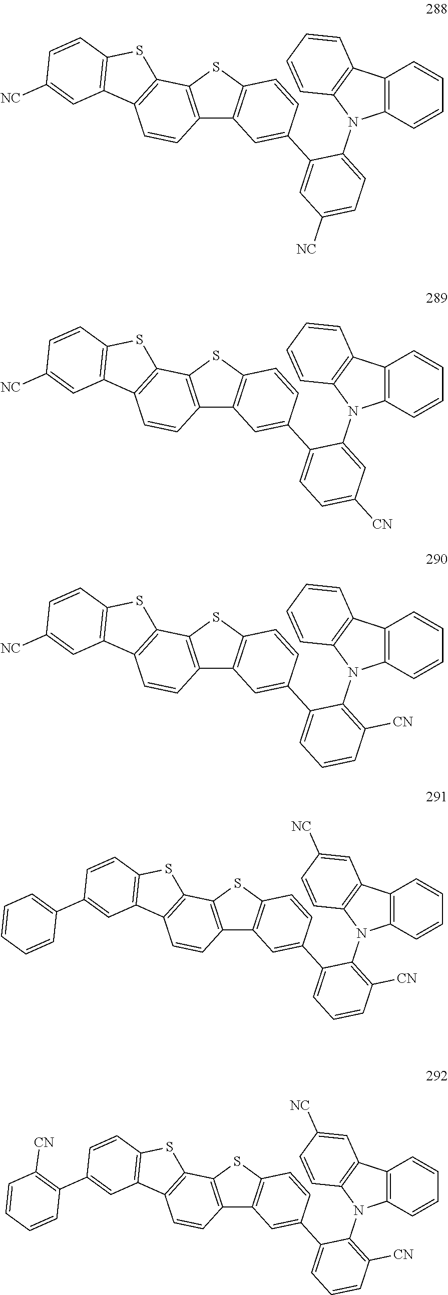 [SMILES: N#Cc1ccc(-n2c3ccccc3c3ccccc32)c(-c2ccc3sc4c(ccc5c6cc(C#N)ccc6sc54)c3c2)c1 ; N#Cc1ccc(-c2ccc3sc4c(ccc5c6cc(C#N)ccc6sc54)c3c2)c(-n2c3ccccc3c3ccccc32)c1 ; N#Cc1ccc2sc3c(ccc4c5cc(-c6cccc(C#N)c6-n6c7ccccc7c7ccccc76)ccc5sc43)c2c1 ; N#Cc1ccc2c(c1)c1ccccc1n2-c1c(C#N)cccc1-c1ccc2sc3c(ccc4c5cc(-c6ccccc6)ccc5sc43)c2c1 ; N#Cc1ccc2c(c1)c1ccccc1n2-c1c(C#N)cccc1-c1ccc2sc3c(ccc4c5cc(-c6ccccc6C#N)ccc5sc43)c2c1]

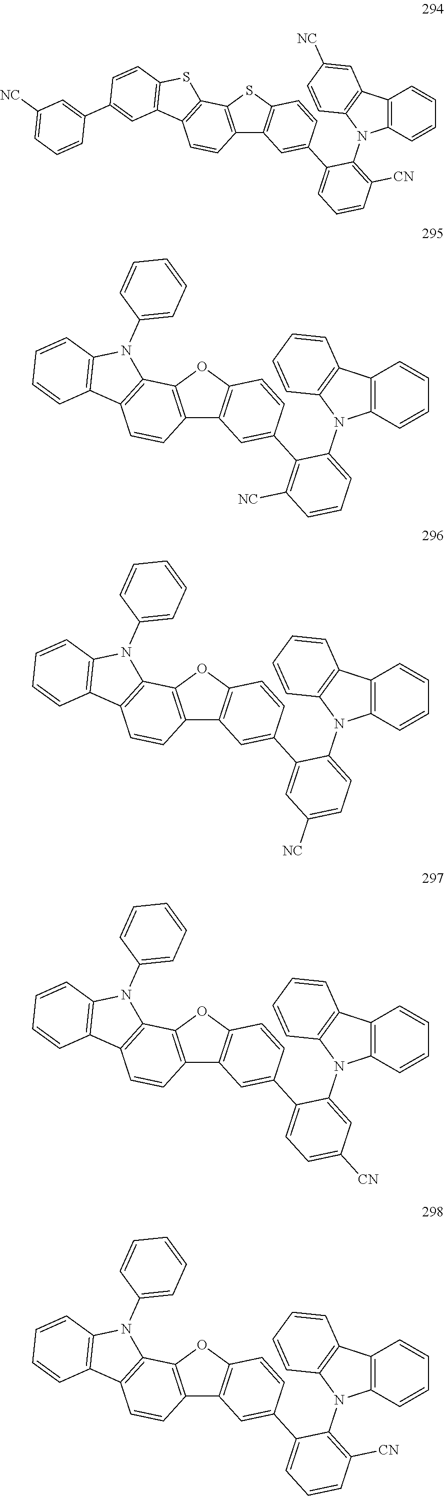 [SMILES: N#Cc1cccc(-c2ccc3sc4c(ccc5c6cc(-c7cccc(C#N)c7-n7c8ccccc8c8cc(C#N)ccc87)ccc6sc54)c3c2)c1 ; N#Cc1cccc(-n2c3ccccc3c3ccccc32)c1-c1ccc2oc3c(ccc4c5ccccc5n(-c5ccccc5)c43)c2c1 ; N#Cc1ccc(-n2c3ccccc3c3ccccc32)c(-c2ccc3oc4c(ccc5c6ccccc6n(-c6ccccc6)c54)c3c2)c1 ; N#Cc1ccc(-c2ccc3oc4c(ccc5c6ccccc6n(-c6ccccc6)c54)c3c2)c(-n2c3ccccc3c3ccccc32)c1 ; N#Cc1cccc(-c2ccc3oc4c(ccc5c6ccccc6n(-c6ccccc6)c54)c3c2)c1-n1c2ccccc2c2ccccc21]

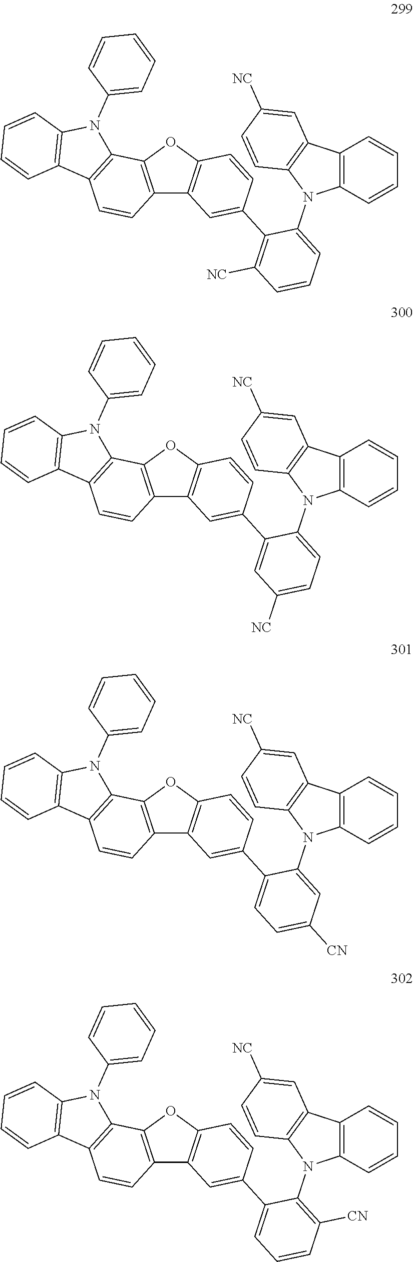 [SMILES: N#Cc1ccc2c(c1)c1ccccc1n2-c1cccc(C#N)c1-c1ccc2oc3c(ccc4c5ccccc5n(-c5ccccc5)c43)c2c1 ; N#Cc1ccc(-n2c3ccccc3c3cc(C#N)ccc32)c(-c2ccc3oc4c(ccc5c6ccccc6n(-c6ccccc6)c54)c3c2)c1 ; N#Cc1ccc(-c2ccc3oc4c(ccc5c6ccccc6n(-c6ccccc6)c54)c3c2)c(-n2c3ccccc3c3cc(C#N)ccc32)c1 ; N#Cc1ccc2c(c1)c1ccccc1n2-c1c(C#N)cccc1-c1ccc2oc3c(ccc4c5ccccc5n(-c5ccccc5)c43)c2c1]

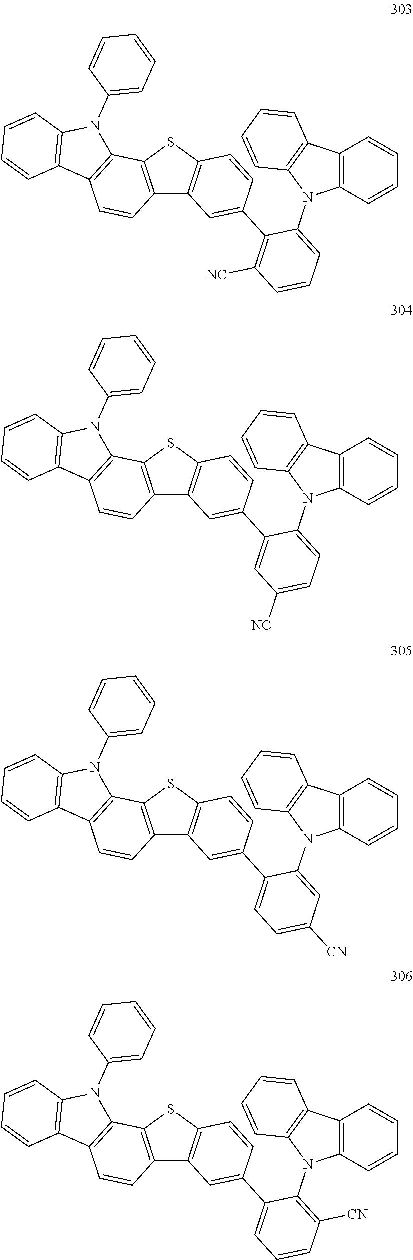 [SMILES: N#Cc1cccc(-n2c3ccccc3c3ccccc32)c1-c1ccc2sc3c(ccc4c5ccccc5n(-c5ccccc5)c43)c2c1 ; N#Cc1ccc(-n2c3ccccc3c3ccccc32)c(-c2ccc3sc4c(ccc5c6ccccc6n(-c6ccccc6)c54)c3c2)c1 ; N#Cc1ccc(-c2ccc3sc4c(ccc5c6ccccc6n(-c6ccccc6)c54)c3c2)c(-n2c3ccccc3c3ccccc32)c1 ; N#Cc1cccc(-c2ccc3sc4c(ccc5c6ccccc6n(-c6ccccc6)c54)c3c2)c1-n1c2ccccc2c2ccccc21]

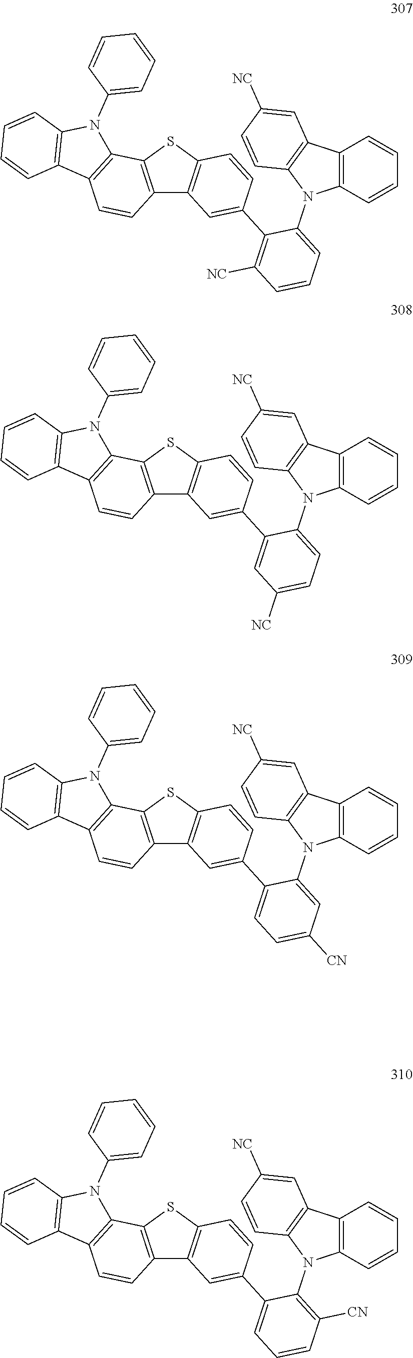 [SMILES: N#Cc1ccc2c(c1)c1ccccc1n2-c1cccc(C#N)c1-c1ccc2sc3c(ccc4c5ccccc5n(-c5ccccc5)c43)c2c1 ; N#Cc1ccc(-n2c3ccccc3c3cc(C#N)ccc32)c(-c2ccc3sc4c(ccc5c6ccccc6n(-c6ccccc6)c54)c3c2)c1 ; N#Cc1ccc(-c2ccc3sc4c(ccc5c6ccccc6n(-c6ccccc6)c54)c3c2)c(-n2c3ccccc3c3cc(C#N)ccc32)c1 ; N#Cc1ccc2c(c1)c1ccccc1n2-c1c(C#N)cccc1-c1ccc2sc3c(ccc4c5ccccc5n(-c5ccccc5)c43)c2c1]

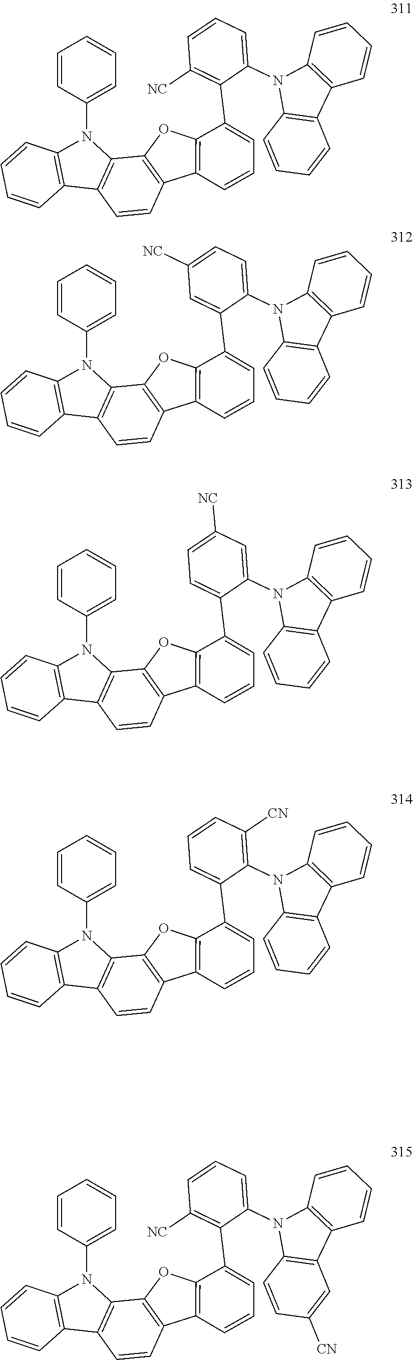

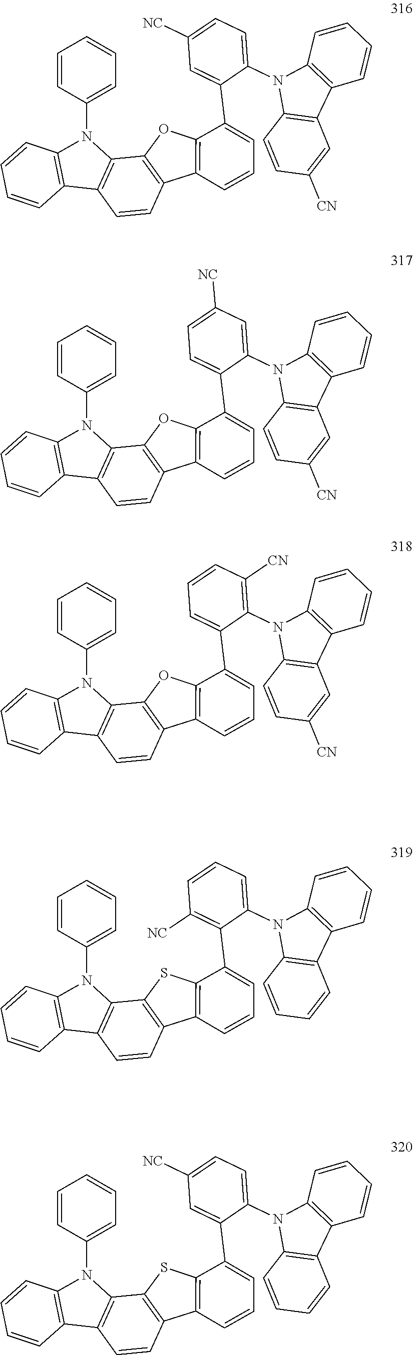 [SMILES: N#Cc1ccc(-n2c3ccccc3c3cc(C#N)ccc32)c(-c2cccc3c2oc2c3ccc3c4ccccc4n(-c4ccccc4)c32)c1 ; N#Cc1ccc(-c2cccc3c2oc2c3ccc3c4ccccc4n(-c4ccccc4)c32)c(-n2c3ccccc3c3cc(C#N)ccc32)c1 ; N#Cc1ccc2c(c1)c1ccccc1n2-c1c(C#N)cccc1-c1cccc2c1oc1c2ccc2c3ccccc3n(-c3ccccc3)c21 ; N#Cc1cccc(-n2c3ccccc3c3ccccc32)c1-c1cccc2c1sc1c2ccc2c3ccccc3n(-c3ccccc3)c21 ; N#Cc1ccc(-n2c3ccccc3c3ccccc32)c(-c2cccc3c2sc2c3ccc3c4ccccc4n(-c4ccccc4)c32)c1]

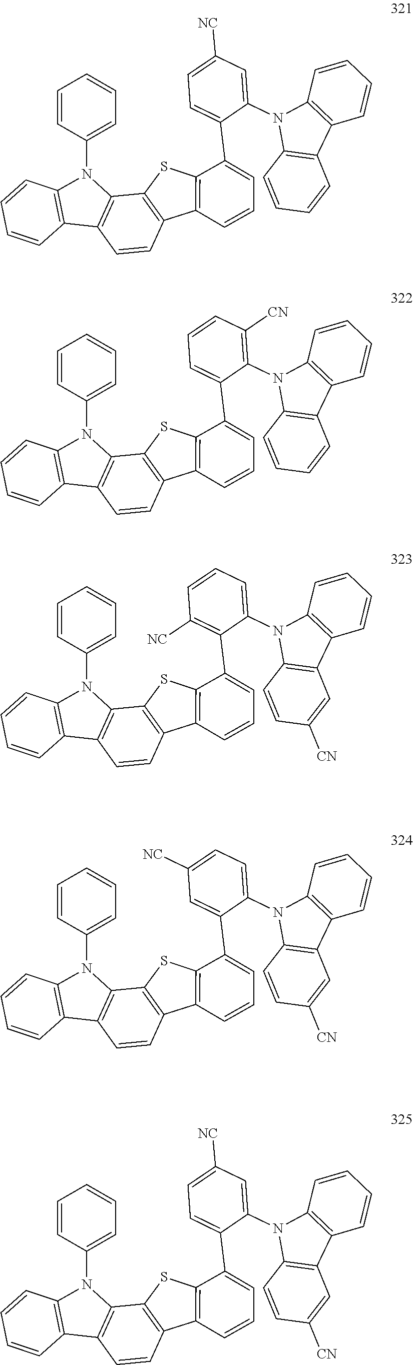 [SMILES: N#Cc1ccc(-c2cccc3c2sc2c3ccc3c4ccccc4n(-c4ccccc4)c32)c(-n2c3ccccc3c3ccccc32)c1 ; N#Cc1cccc(-c2cccc3c2sc2c3ccc3c4ccccc4n(-c4ccccc4)c32)c1-n1c2ccccc2c2ccccc21 ; N#Cc1ccc2c(c1)c1ccccc1n2-c1cccc(C#N)c1-c1cccc2c1sc1c2ccc2c3ccccc3n(-c3ccccc3)c21 ; N#Cc1ccc(-n2c3ccccc3c3cc(C#N)ccc32)c(-c2cccc3c2sc2c3ccc3c4ccccc4n(-c4ccccc4)c32)c1 ; N#Cc1ccc(-c2cccc3c2sc2c3ccc3c4ccccc4n(-c4ccccc4)c32)c(-n2c3ccccc3c3cc(C#N)ccc32)c1]

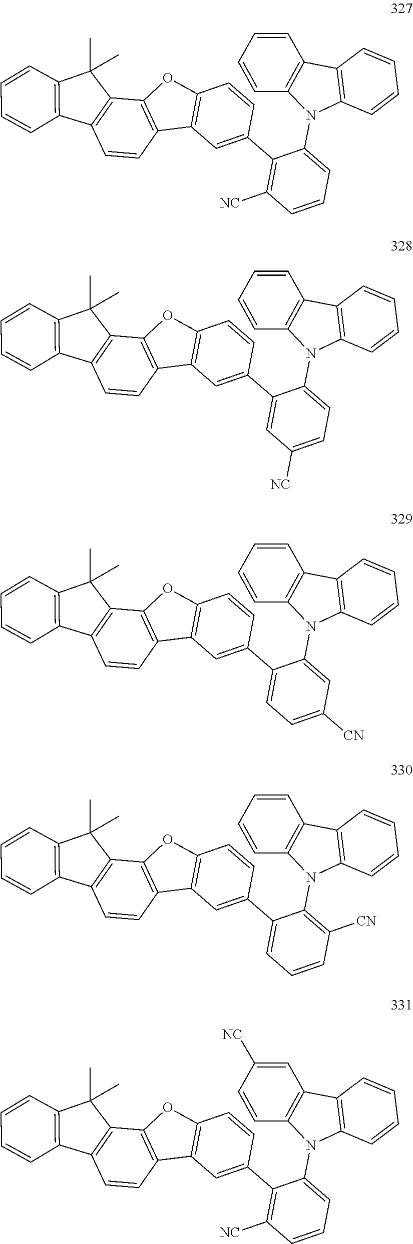 [SMILES: CC1(C)c2ccccc2-c2ccc3c(oc4ccc(-c5c(C#N)cccc5-n5c6ccccc6c6ccccc65)cc43)c21 ; CC1(C)c2ccccc2-c2ccc3c(oc4ccc(-c5cc(C#N)ccc5-n5c6ccccc6c6ccccc65)cc43)c21 ; CC1(C)c2ccccc2-c2ccc3c(oc4ccc(-c5ccc(C#N)cc5-n5c6ccccc6c6ccccc65)cc43)c21 ; CC1(C)c2ccccc2-c2ccc3c(oc4ccc(-c5cccc(C#N)c5-n5c6ccccc6c6ccccc65)cc43)c21 ; CC1(C)c2ccccc2-c2ccc3c(oc4ccc(-c5c(C#N)cccc5-n5c6ccccc6c6cc(C#N)ccc65)cc43)c21]

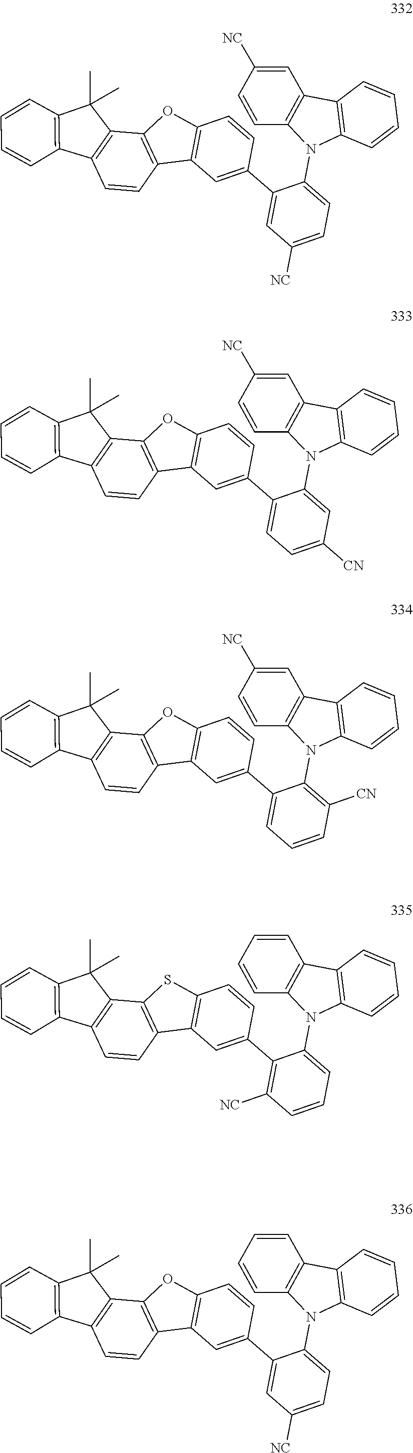 [SMILES: CC1(C)c2ccccc2-c2ccc3c(oc4ccc(-c5cc(C#N)ccc5-n5c6ccccc6c6cc(C#N)ccc65)cc43)c21 ; CC1(C)c2ccccc2-c2ccc3c(oc4ccc(-c5ccc(C#N)cc5-n5c6ccccc6c6cc(C#N)ccc65)cc43)c21 ; CC1(C)c2ccccc2-c2ccc3c(oc4ccc(-c5cccc(C#N)c5-n5c6ccccc6c6cc(C#N)ccc65)cc43)c21 ; CC1(C)c2ccccc2-c2ccc3c(sc4ccc(-c5c(C#N)cccc5-n5c6ccccc6c6ccccc65)cc43)c21 ; CC1(C)c2ccccc2-c2ccc3c(oc4ccc(-c5cc(C#N)ccc5-n5c6ccccc6c6ccccc65)cc43)c21]

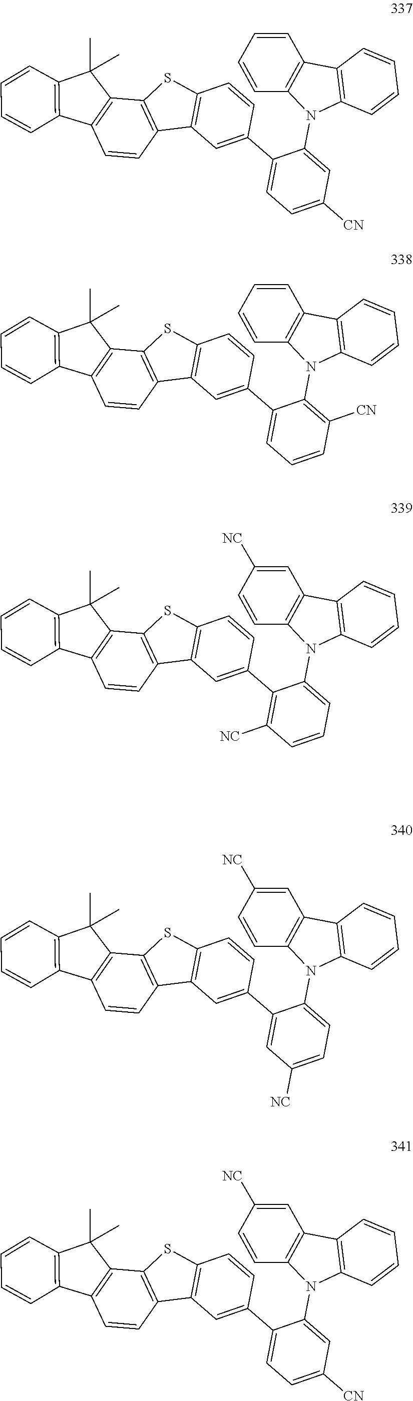 [SMILES: CC1(C)c2ccccc2-c2ccc3c(sc4ccc(-c5ccc(C#N)cc5-n5c6ccccc6c6ccccc65)cc43)c21 ; CC1(C)c2ccccc2-c2ccc3c(sc4ccc(-c5cccc(C#N)c5-n5c6ccccc6c6ccccc65)cc43)c21 ; CC1(C)c2ccccc2-c2ccc3c(sc4ccc(-c5c(C#N)cccc5-n5c6ccccc6c6cc(C#N)ccc65)cc43)c21 ; CC1(C)c2ccccc2-c2ccc3c(sc4ccc(-c5cc(C#N)ccc5-n5c6ccccc6c6cc(C#N)ccc65)cc43)c21 ; CC1(C)c2ccccc2-c2ccc3c(sc4ccc(-c5ccc(C#N)cc5-n5c6ccccc6c6cc(C#N)ccc65)cc43)c21]

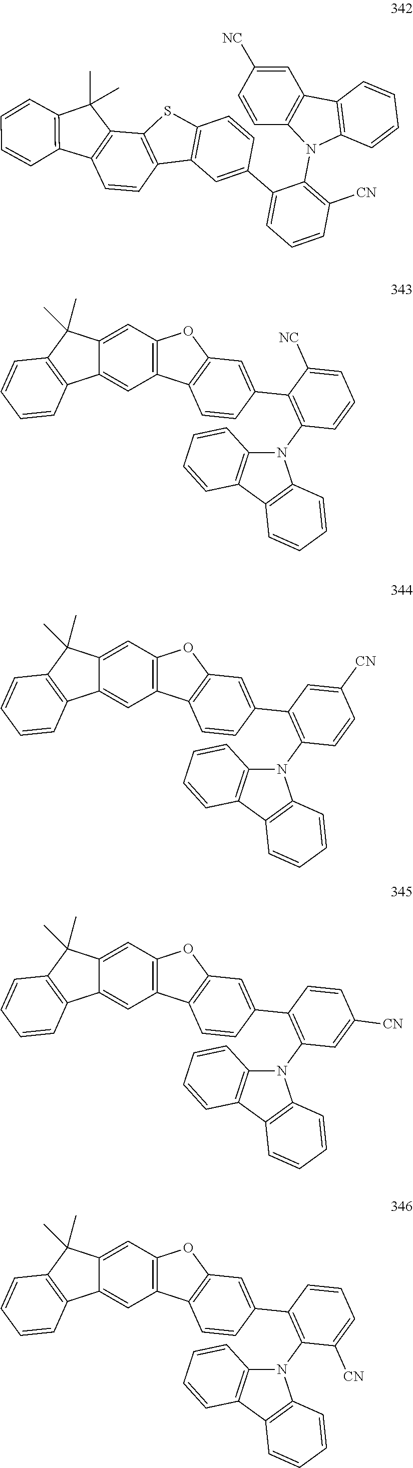 [SMILES: CC1(C)c2ccccc2-c2ccc3c(sc4ccc(-c5cccc(C#N)c5-n5c6ccccc6c6cc(C#N)ccc65)cc43)c21 ; CC1(C)c2ccccc2-c2cc3c(cc21)oc1cc(-c2c(C#N)cccc2-n2c4ccccc4c4ccccc42)ccc13 ; CC1(C)c2ccccc2-c2cc3c(cc21)oc1cc(-c2cc(C#N)ccc2-n2c4ccccc4c4ccccc42)ccc13 ; CC1(C)c2ccccc2-c2cc3c(cc21)oc1cc(-c2ccc(C#N)cc2-n2c4ccccc4c4ccccc42)ccc13 ; CC1(C)c2ccccc2-c2cc3c(cc21)oc1cc(-c2cccc(C#N)c2-n2c4ccccc4c4ccccc42)ccc13]

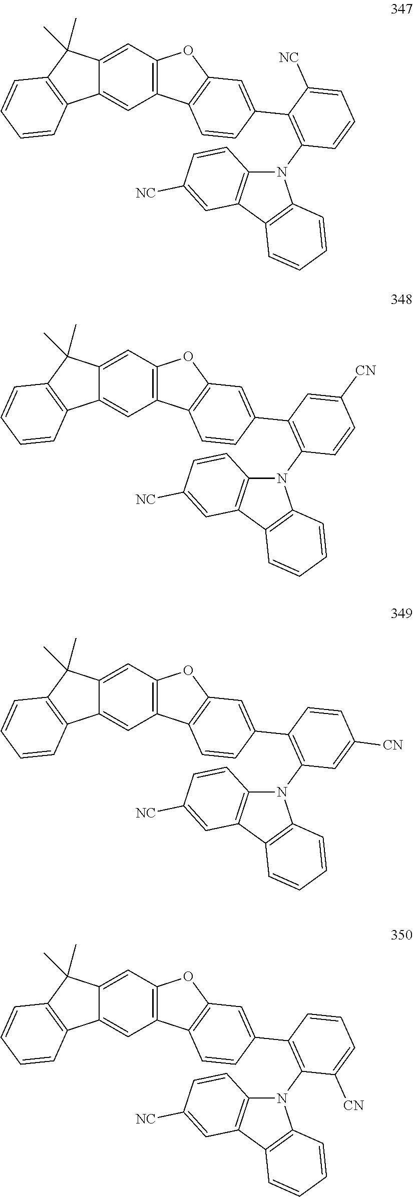 [SMILES: CC1(C)c2ccccc2-c2cc3c(cc21)oc1cc(-c2c(C#N)cccc2-n2c4ccccc4c4cc(C#N)ccc42)ccc13 ; CC1(C)c2ccccc2-c2cc3c(cc21)oc1cc(-c2cc(C#N)ccc2-n2c4ccccc4c4cc(C#N)ccc42)ccc13 ; CC1(C)c2ccccc2-c2cc3c(cc21)oc1cc(-c2ccc(C#N)cc2-n2c4ccccc4c4cc(C#N)ccc42)ccc13 ; CC1(C)c2ccccc2-c2cc3c(cc21)oc1cc(-c2cccc(C#N)c2-n2c4ccccc4c4cc(C#N)ccc42)ccc13]

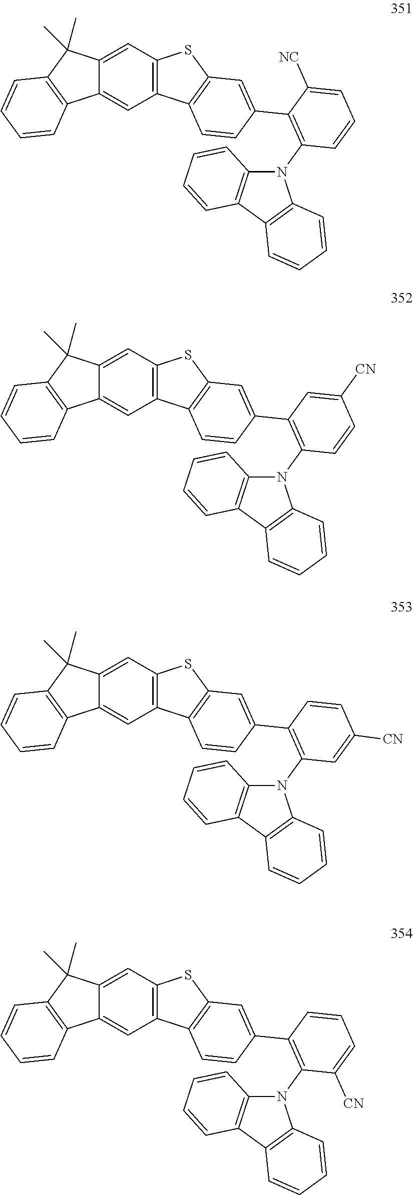 [SMILES: CC1(C)c2ccccc2-c2cc3c(cc21)sc1cc(-c2c(C#N)cccc2-n2c4ccccc4c4ccccc42)ccc13 ; CC1(C)c2ccccc2-c2cc3c(cc21)sc1cc(-c2cc(C#N)ccc2-n2c4ccccc4c4ccccc42)ccc13 ; CC1(C)c2ccccc2-c2cc3c(cc21)sc1cc(-c2ccc(C#N)cc2-n2c4ccccc4c4ccccc42)ccc13 ; CC1(C)c2ccccc2-c2cc3c(cc21)sc1cc(-c2cccc(C#N)c2-n2c4ccccc4c4ccccc42)ccc13]

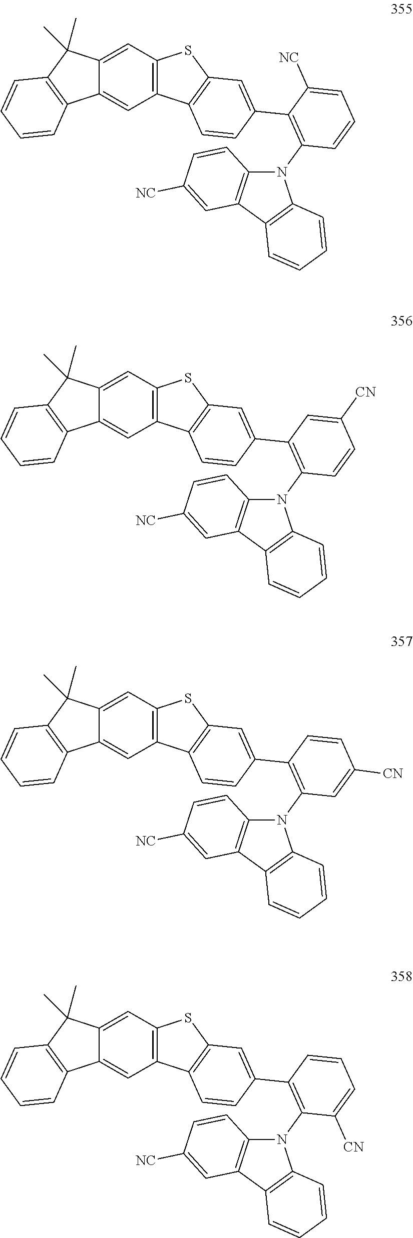 [SMILES: CC1(C)c2ccccc2-c2cc3c(cc21)sc1cc(-c2c(C#N)cccc2-n2c4ccccc4c4cc(C#N)ccc42)ccc13 ; CC1(C)c2ccccc2-c2cc3c(cc21)sc1cc(-c2cc(C#N)ccc2-n2c4ccccc4c4cc(C#N)ccc42)ccc13 ; CC1(C)c2ccccc2-c2cc3c(cc21)sc1cc(-c2ccc(C#N)cc2-n2c4ccccc4c4cc(C#N)ccc42)ccc13 ; CC1(C)c2ccccc2-c2cc3c(cc21)sc1cc(-c2cccc(C#N)c2-n2c4ccccc4c4cc(C#N)ccc42)ccc13]

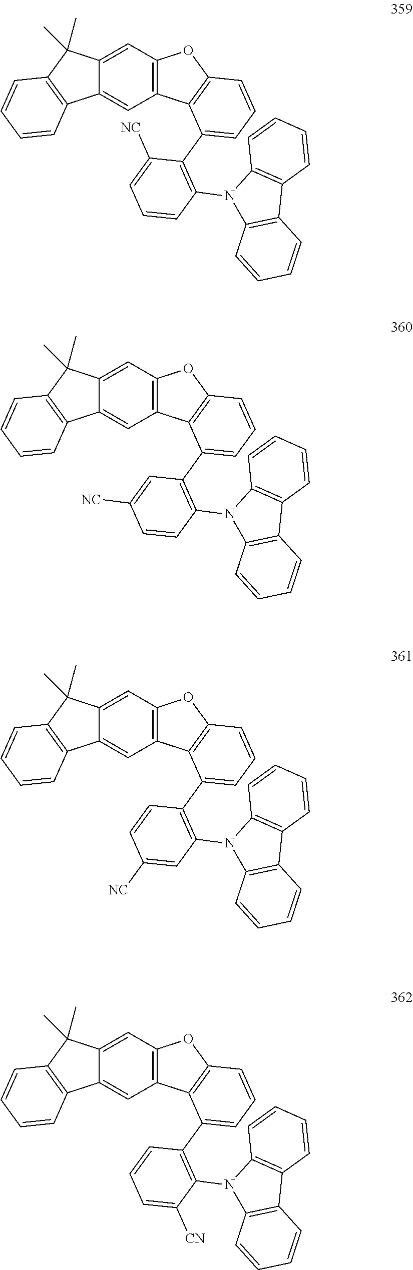 [SMILES: CC1(C)c2ccccc2-c2cc3c(cc21)oc1cccc(-c2c(C#N)cccc2-n2c4ccccc4c4ccccc42)c13 ; CC1(C)c2ccccc2-c2cc3c(cc21)oc1cccc(-c2cc(C#N)ccc2-n2c4ccccc4c4ccccc42)c13 ; CC1(C)c2ccccc2-c2cc3c(cc21)oc1cccc(-c2ccc(C#N)cc2-n2c4ccccc4c4ccccc42)c13 ; CC1(C)c2ccccc2-c2cc3c(cc21)oc1cccc(-c2cccc(C#N)c2-n2c4ccccc4c4ccccc42)c13]

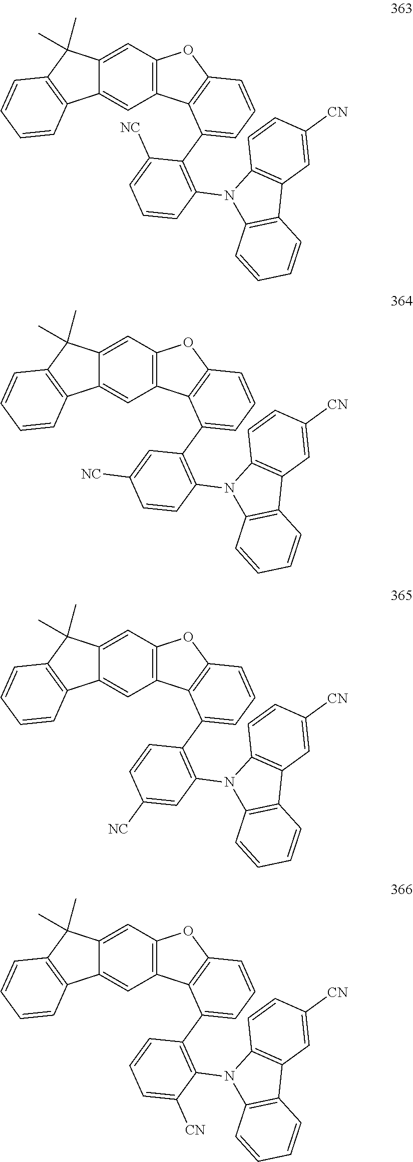 [SMILES: CC1(C)c2ccccc2-c2cc3c(cc21)oc1cccc(-c2c(C#N)cccc2-n2c4ccccc4c4cc(C#N)ccc42)c13 ; CC1(C)c2ccccc2-c2cc3c(cc21)oc1cccc(-c2cc(C#N)ccc2-n2c4ccccc4c4cc(C#N)ccc42)c13 ; CC1(C)c2ccccc2-c2cc3c(cc21)oc1cccc(-c2ccc(C#N)cc2-n2c4ccccc4c4cc(C#N)ccc42)c13 ; CC1(C)c2ccccc2-c2cc3c(cc21)oc1cccc(-c2cccc(C#N)c2-n2c4ccccc4c4cc(C#N)ccc42)c13]

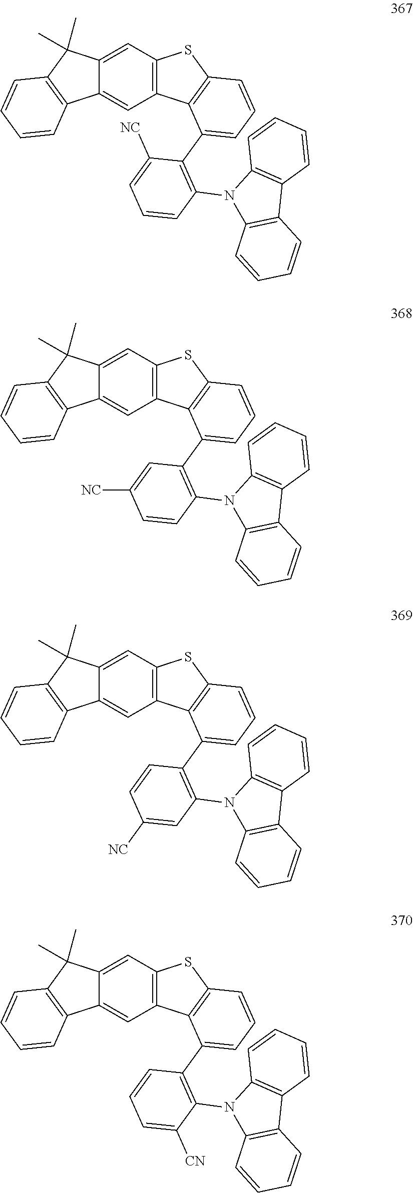 [SMILES: CC1(C)c2ccccc2-c2cc3c(cc21)sc1cccc(-c2c(C#N)cccc2-n2c4ccccc4c4ccccc42)c13 ; CC1(C)c2ccccc2-c2cc3c(cc21)sc1cccc(-c2cc(C#N)ccc2-n2c4ccccc4c4ccccc42)c13 ; CC1(C)c2ccccc2-c2cc3c(cc21)sc1cccc(-c2ccc(C#N)cc2-n2c4ccccc4c4ccccc42)c13 ; CC1(C)c2ccccc2-c2cc3c(cc21)sc1cccc(-c2cccc(C#N)c2-n2c4ccccc4c4ccccc42)c13]

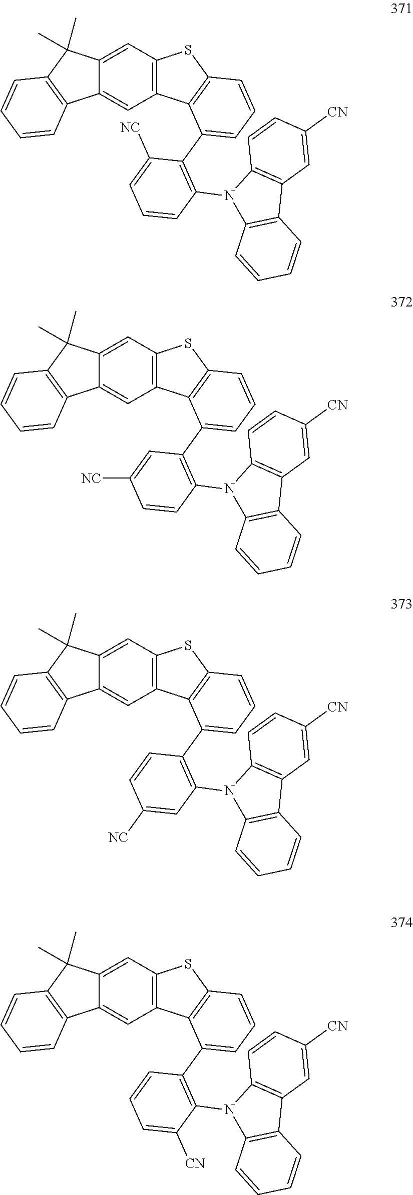 [SMILES: CC1(C)c2ccccc2-c2cc3c(cc21)sc1cccc(-c2c(C#N)cccc2-n2c4ccccc4c4cc(C#N)ccc42)c13 ; CC1(C)c2ccccc2-c2cc3c(cc21)sc1cccc(-c2cc(C#N)ccc2-n2c4ccccc4c4cc(C#N)ccc42)c13 ; CC1(C)c2ccccc2-c2cc3c(cc21)sc1cccc(-c2ccc(C#N)cc2-n2c4ccccc4c4cc(C#N)ccc42)c13 ; CC1(C)c2ccccc2-c2cc3c(cc21)sc1cccc(-c2cccc(C#N)c2-n2c4ccccc4c4cc(C#N)ccc42)c13]

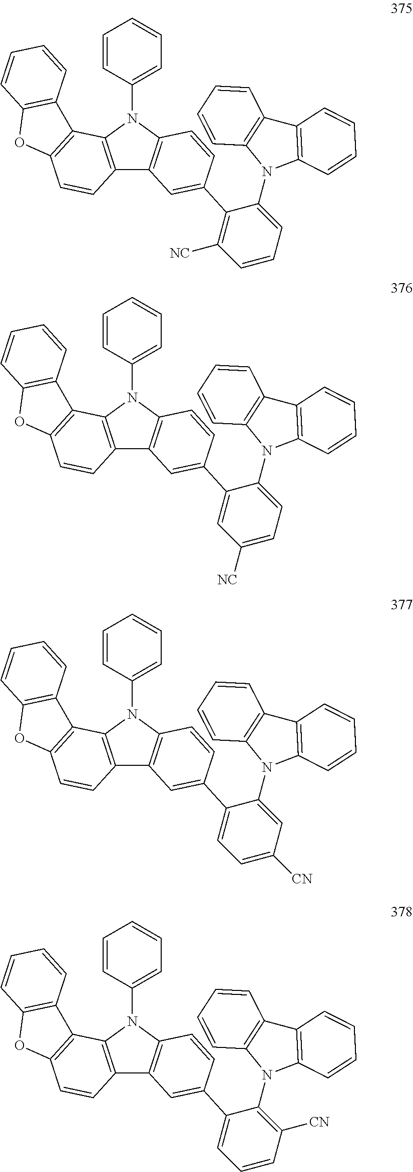 [SMILES: N#Cc1cccc(-n2c3ccccc3c3ccccc32)c1-c1ccc2c(c1)c1ccc3oc4ccccc4c3c1n2-c1ccccc1 ; N#Cc1ccc(-n2c3ccccc3c3ccccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3ccccc32)c1 ; N#Cc1cccc(-c2ccc3c(c2)c2ccc4oc5ccccc5c4c2n3-c2ccccc2)c1-n1c2ccccc2c2ccccc21]

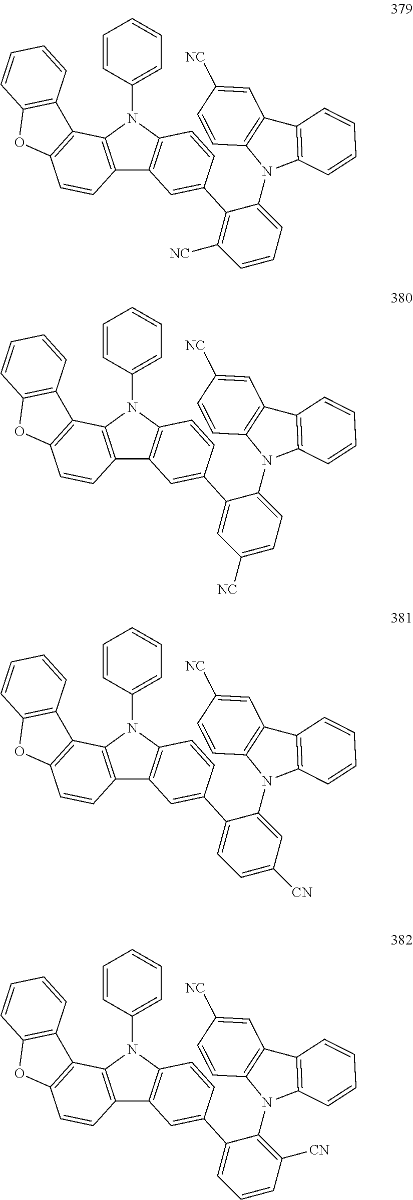 [SMILES: N#Cc1ccc2c(c1)c1ccccc1n2-c1cccc(C#N)c1-c1ccc2c(c1)c1ccc3oc4ccccc4c3c1n2-c1ccccc1 ; N#Cc1ccc(-n2c3ccccc3c3cc(C#N)ccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3oc4ccccc4c3c1n2-c1ccccc1]

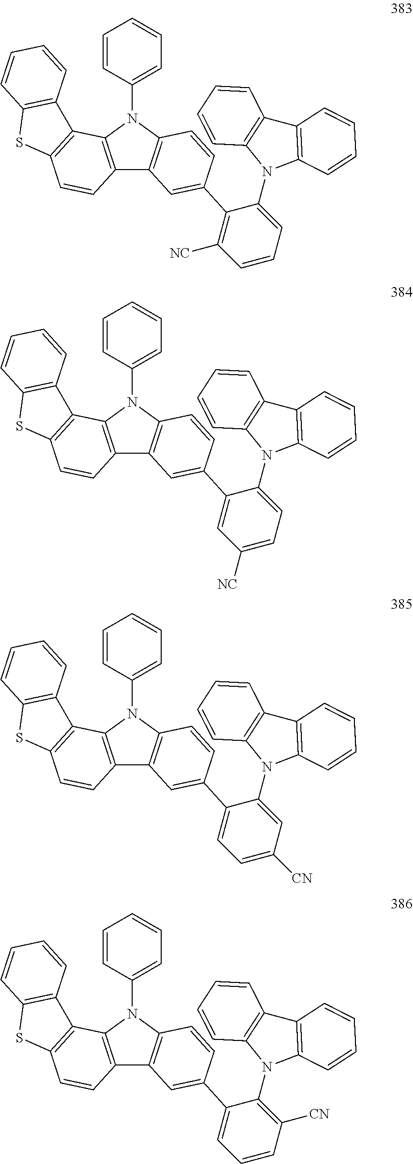 [SMILES: N#Cc1cccc(-n2c3ccccc3c3ccccc32)c1-c1ccc2c(c1)c1ccc3sc4ccccc4c3c1n2-c1ccccc1 ; N#Cc1ccc(-n2c3ccccc3c3ccccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3ccccc32)c1 ; N#Cc1cccc(-c2ccc3c(c2)c2ccc4sc5ccccc5c4c2n3-c2ccccc2)c1-n1c2ccccc2c2ccccc21]

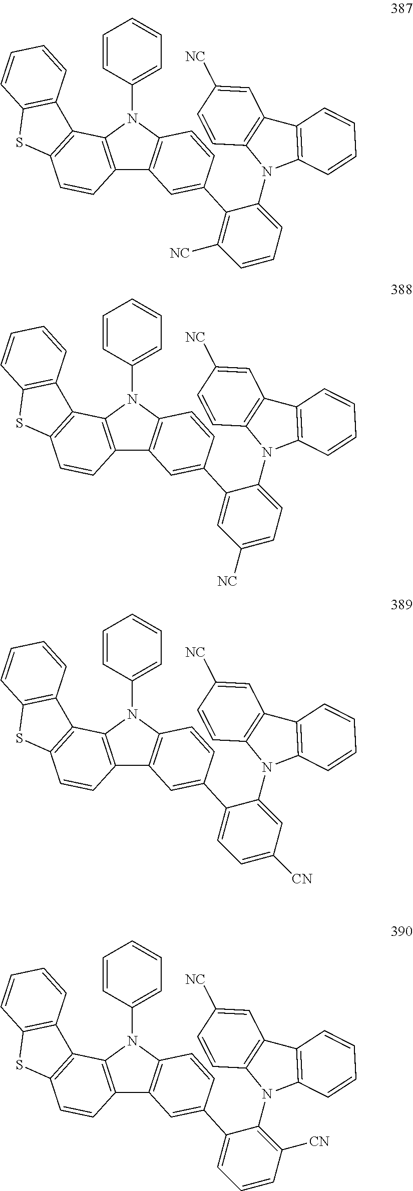 [SMILES: N#Cc1ccc2c(c1)c1ccccc1n2-c1cccc(C#N)c1-c1ccc2c(c1)c1ccc3sc4ccccc4c3c1n2-c1ccccc1 ; N#Cc1ccc(-n2c3ccccc3c3cc(C#N)ccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3sc4ccccc4c3c1n2-c1ccccc1]

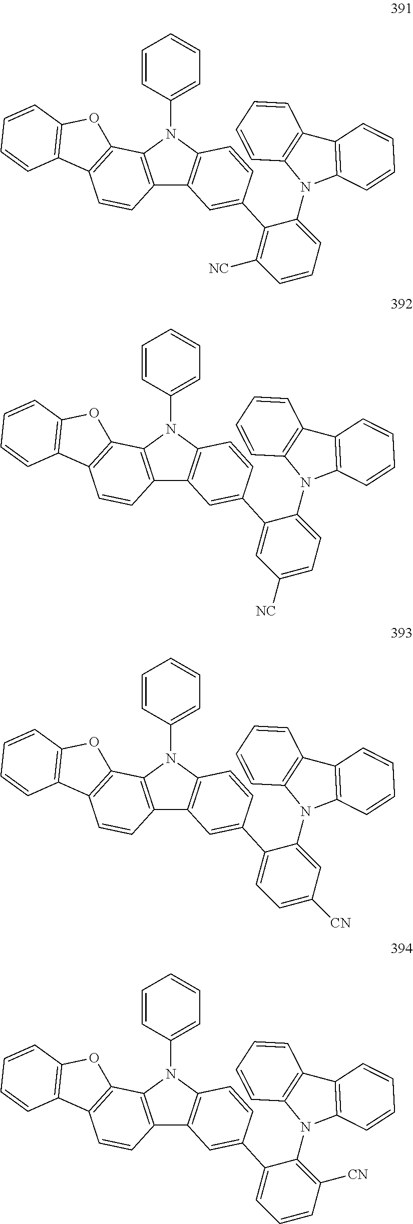 [SMILES: N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21]

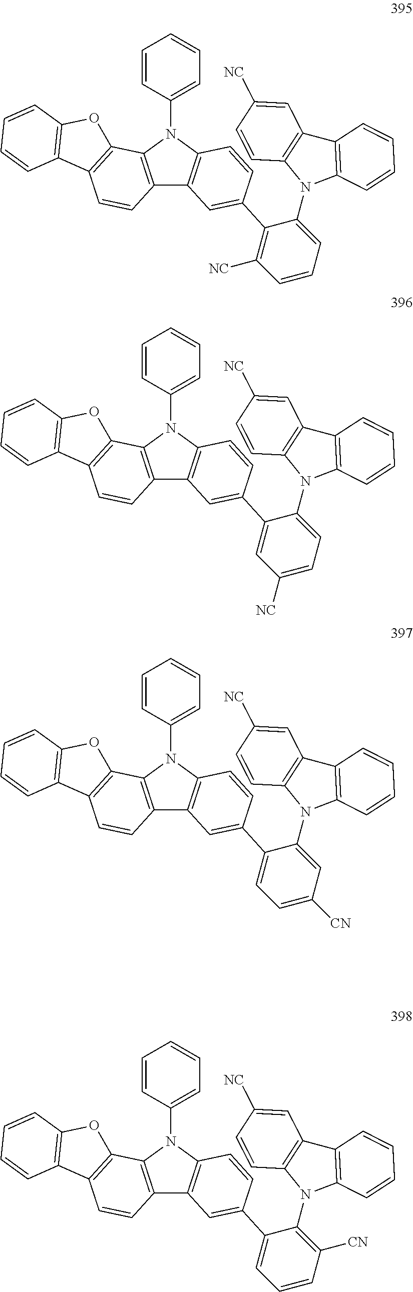 [SMILES: N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3c4ccccc4oc3c1n2-c1ccccc1]

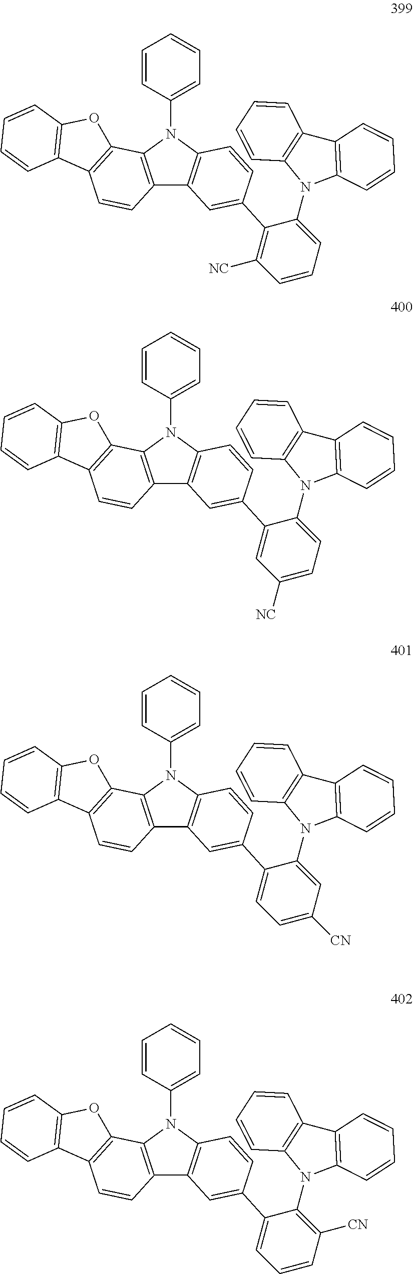 [SMILES: N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21]

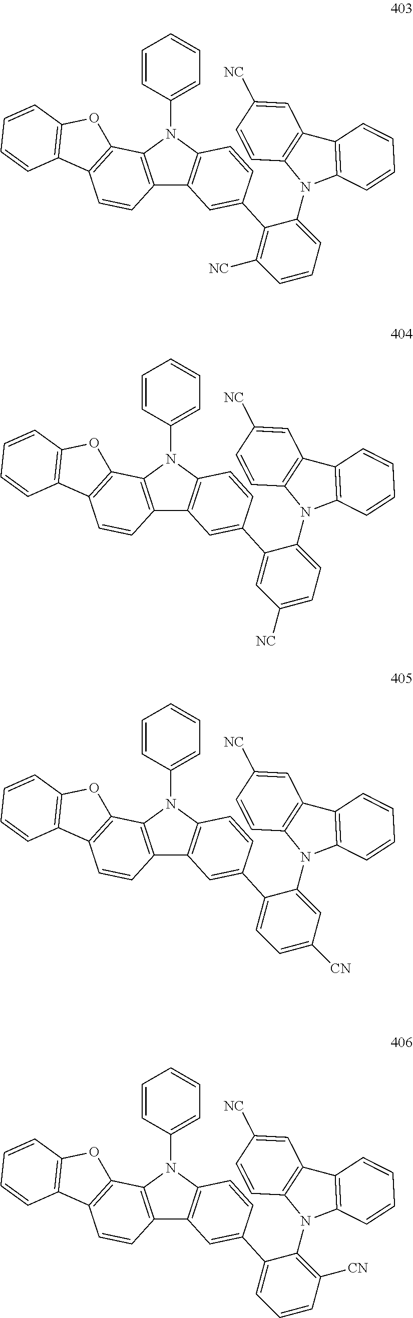 [SMILES: N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3c4ccccc4oc3c1n2-c1ccccc1]

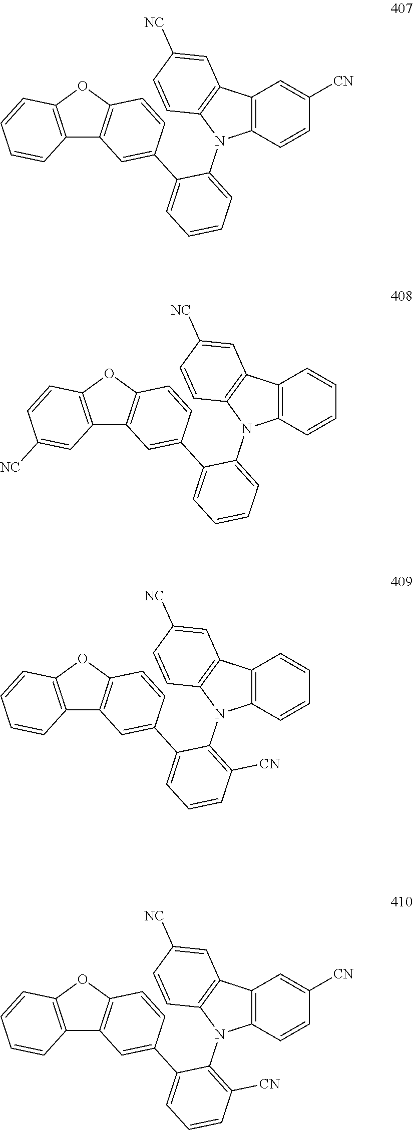 [SMILES: N#Cc1ccc2c(c1)c1cc(C#N)ccc1n2-c1ccccc1-c1ccc2oc3ccccc3c2c1 ; N#Cc1ccc2oc3ccc(-c4ccccc4-n4c5ccccc5c5cc(C#N)ccc54)cc3c2c1 ; N#Cc1ccc2c(c1)c1ccccc1n2-c1c(C#N)cccc1-c1ccc2oc3ccccc3c2c1 ; N#Cc1ccc2c(c1)c1cc(C#N)ccc1n2-c1c(C#N)cccc1-c1ccc2oc3ccccc3c2c1]

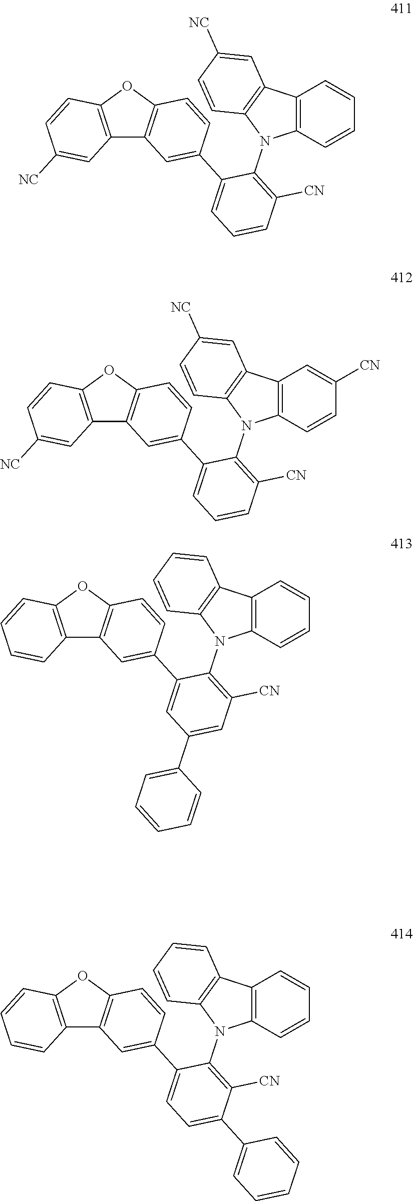

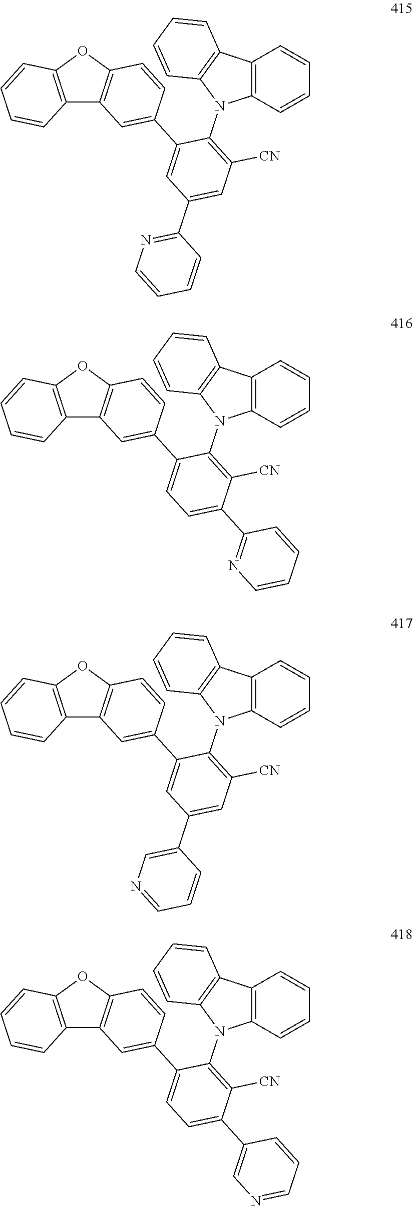

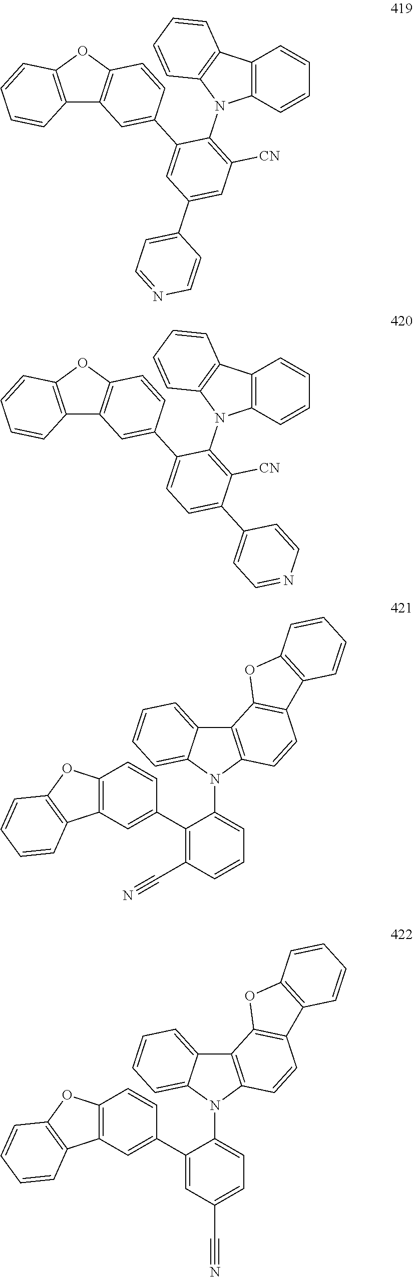

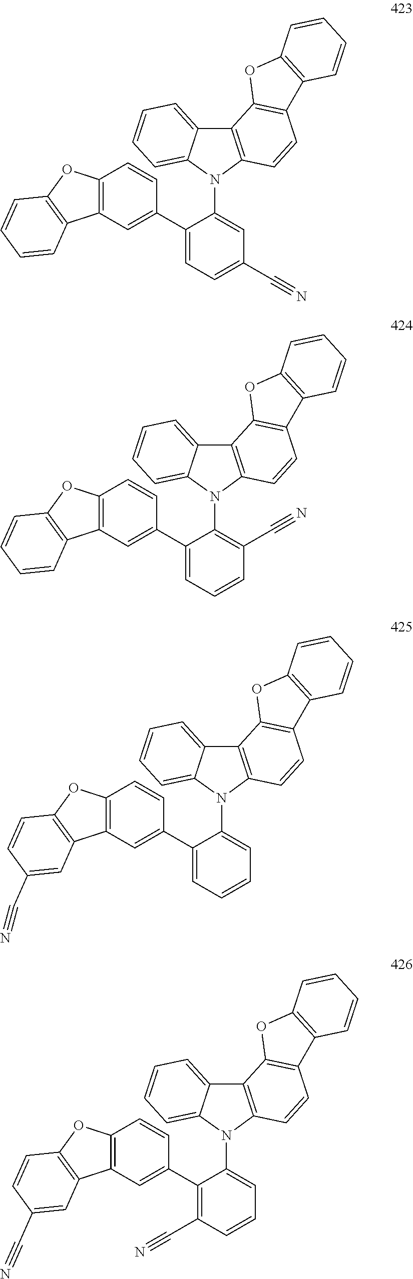

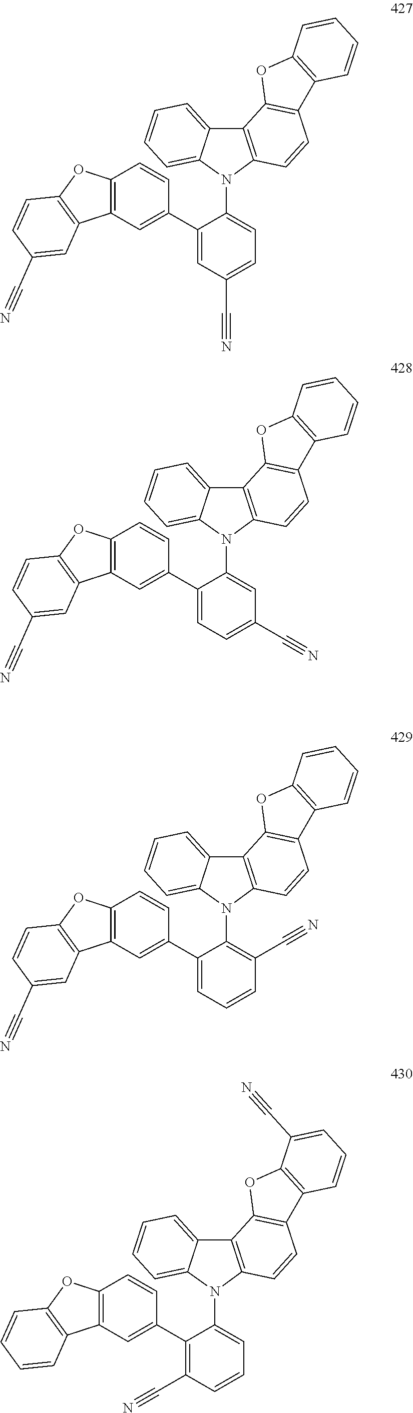 [SMILES: N#Cc1ccc(-n2c3ccccc3c3c4oc5ccccc5c4ccc32)c(-c2ccc3oc4ccc(C#N)cc4c3c2)c1 ; N#Cc1ccc(-c2ccc3oc4ccc(C#N)cc4c3c2)c(-n2c3ccccc3c3c4oc5ccccc5c4ccc32)c1 ; N#Cc1ccc2oc3ccc(-c4cccc(C#N)c4-n4c5ccccc5c5c6oc7ccccc7c6ccc54)cc3c2c1 ; N#Cc1cccc(-n2c3ccccc3c3c4oc5c(C#N)cccc5c4ccc32)c1-c1ccc2oc3ccccc3c2c1]

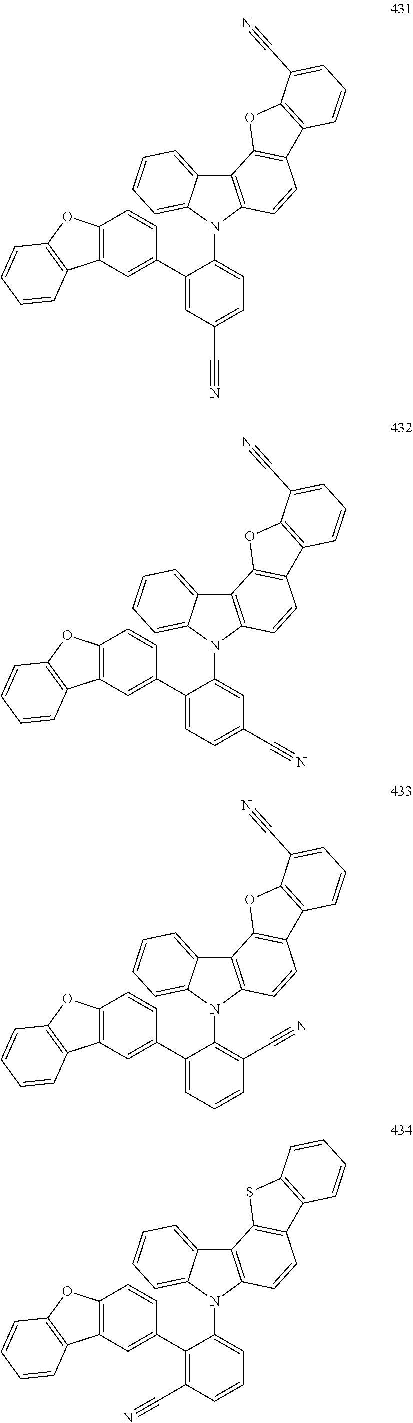 [SMILES: N#Cc1ccc(-n2c3ccccc3c3c4oc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4oc5c(C#N)cccc5c4ccc32)c1 ; N#Cc1cccc(-c2ccc3oc4ccccc4c3c2)c1-n1c2ccccc2c2c3oc4c(C#N)cccc4c3ccc21 ; N#Cc1cccc(-n2c3ccccc3c3c4sc5ccccc5c4ccc32)c1-c1ccc2oc3ccccc3c2c1]

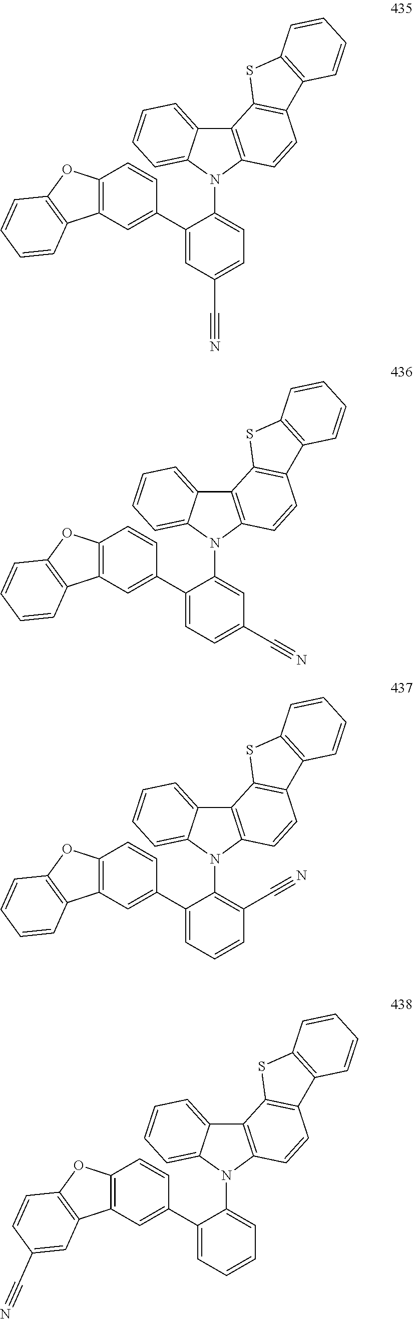

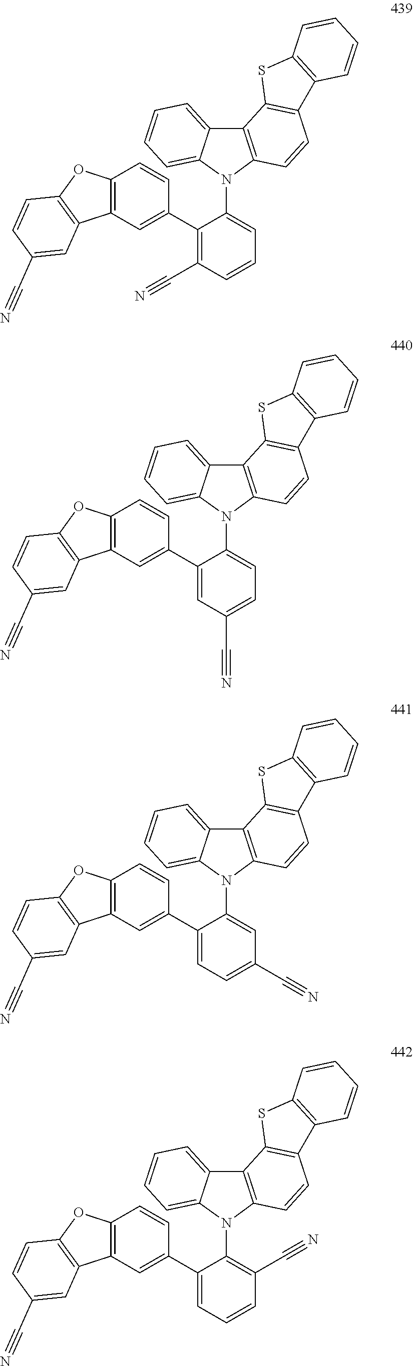 [SMILES: N#Cc1ccc2oc3ccc(-c4c(C#N)cccc4-n4c5ccccc5c5c6sc7ccccc7c6ccc54)cc3c2c1 ; N#Cc1ccc(-n2c3ccccc3c3c4sc5ccccc5c4ccc32)c(-c2ccc3oc4ccc(C#N)cc4c3c2)c1 ; N#Cc1ccc(-c2ccc3oc4ccc(C#N)cc4c3c2)c(-n2c3ccccc3c3c4sc5ccccc5c4ccc32)c1 ; N#Cc1ccc2oc3ccc(-c4cccc(C#N)c4-n4c5ccccc5c5c6sc7ccccc7c6ccc54)cc3c2c1]

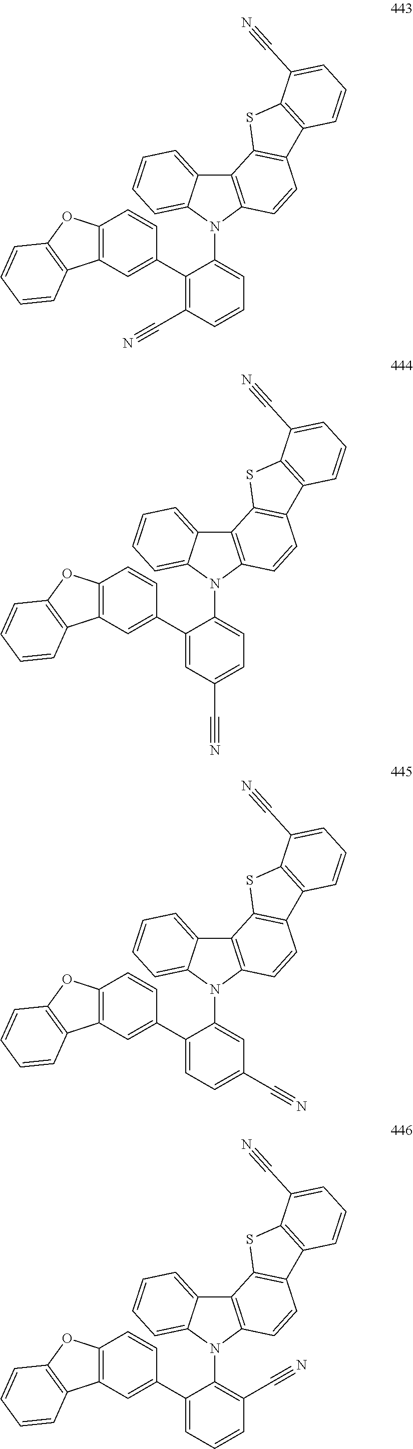 [SMILES: N#Cc1cccc(-n2c3ccccc3c3c4sc5c(C#N)cccc5c4ccc32)c1-c1ccc2oc3ccccc3c2c1 ; N#Cc1ccc(-n2c3ccccc3c3c4sc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4sc5c(C#N)cccc5c4ccc32)c1 ; N#Cc1cccc(-c2ccc3oc4ccccc4c3c2)c1-n1c2ccccc2c2c3sc4c(C#N)cccc4c3ccc21]

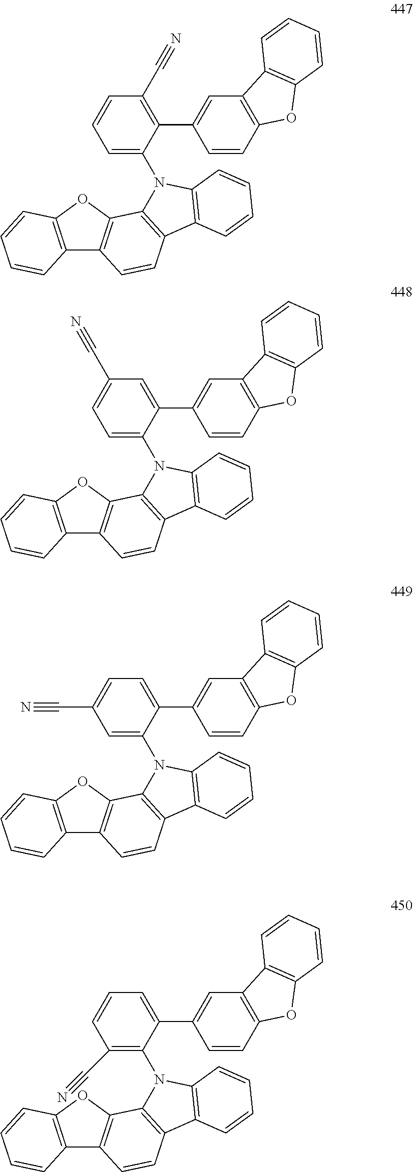 [SMILES: N#Cc1cccc(-n2c3ccccc3c3ccc4c5ccccc5oc4c32)c1-c1ccc2oc3ccccc3c2c1 ; N#Cc1ccc(-n2c3ccccc3c3ccc4c5ccccc5oc4c32)c(-c2ccc3oc4ccccc4c3c2)c1 ; N#Cc1ccc(-c2ccc3oc4ccccc4c3c2)c(-n2c3ccccc3c3ccc4c5ccccc5oc4c32)c1 ; N#Cc1cccc(-c2ccc3oc4ccccc4c3c2)c1-n1c2ccccc2c2ccc3c4ccccc4oc3c21]

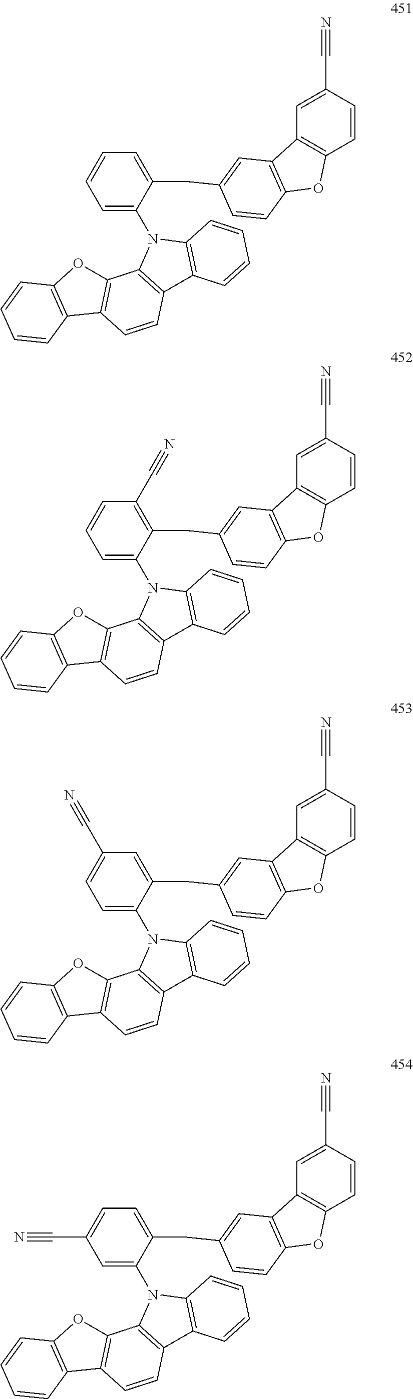 [SMILES: N#Cc1ccc2oc3ccc(Cc4ccccc4-n4c5ccccc5c5ccc6c7ccccc7oc6c54)cc3c2c1 ; N#Cc1ccc2oc3ccc(Cc4c(C#N)cccc4-n4c5ccccc5c5ccc6c7ccccc7oc6c54)cc3c2c1 ; N#Cc1ccc(-n2c3ccccc3c3ccc4c5ccccc5oc4c32)c(Cc2ccc3oc4ccc(C#N)cc4c3c2)c1 ; N#Cc1ccc(Cc2ccc3oc4ccc(C#N)cc4c3c2)c(-n2c3ccccc3c3ccc4c5ccccc5oc4c32)c1]

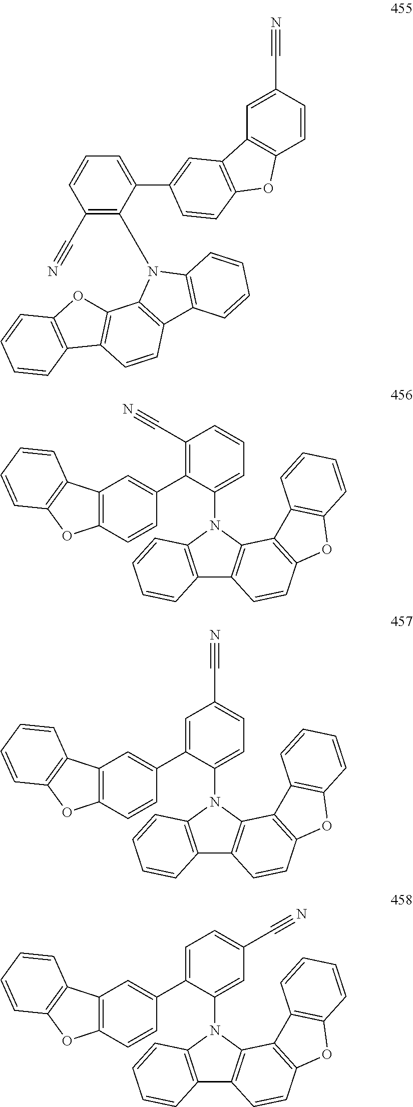

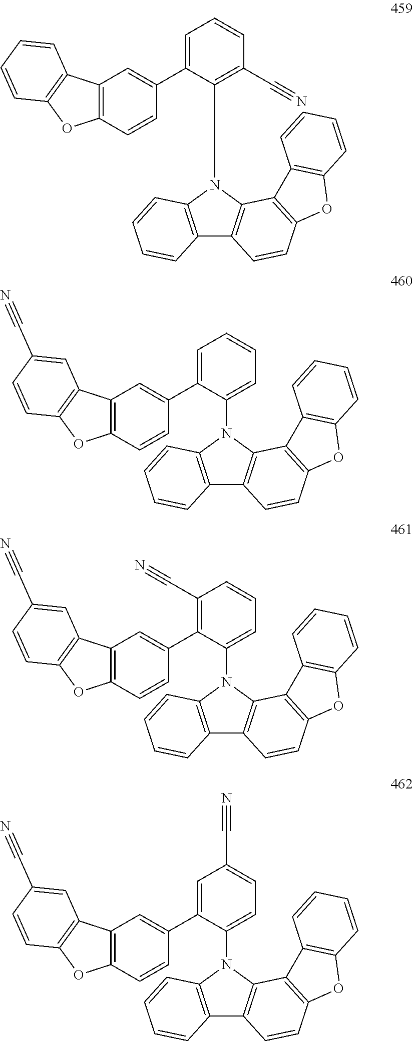 [SMILES: N#Cc1cccc(-c2ccc3oc4ccccc4c3c2)c1CCn1c2ccccc2c2ccc3oc4ccccc4c3c21 ; N#Cc1ccc2oc3ccc(-c4ccccc4-n4c5ccccc5c5ccc6oc7ccccc7c6c54)cc3c2c1 ; N#Cc1ccc2oc3ccc(-c4c(C#N)cccc4-n4c5ccccc5c5ccc6oc7ccccc7c6c54)cc3c2c1 ; N#Cc1ccc(-n2c3ccccc3c3ccc4oc5ccccc5c4c32)c(-c2ccc3oc4ccc(C#N)cc4c3c2)c1]

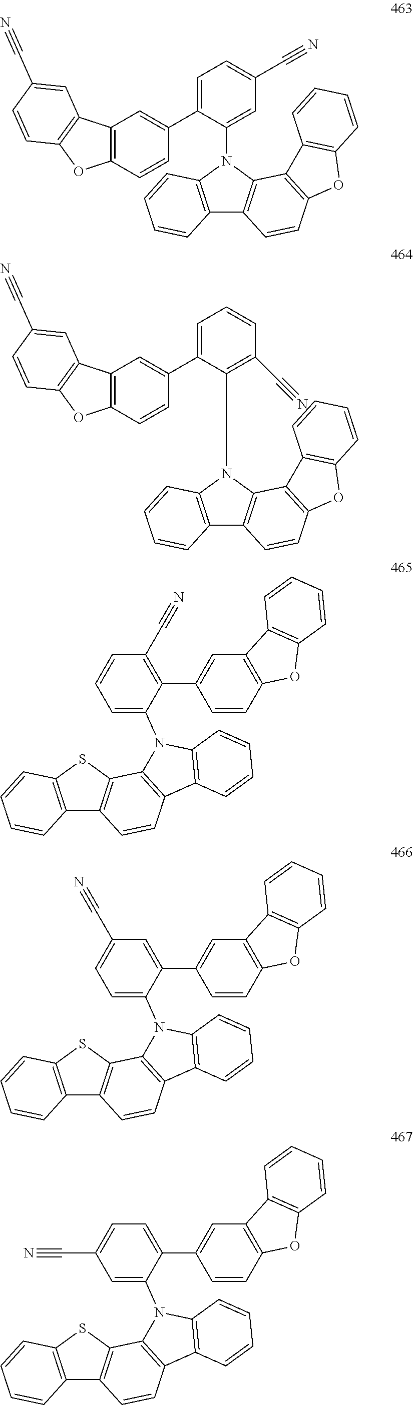

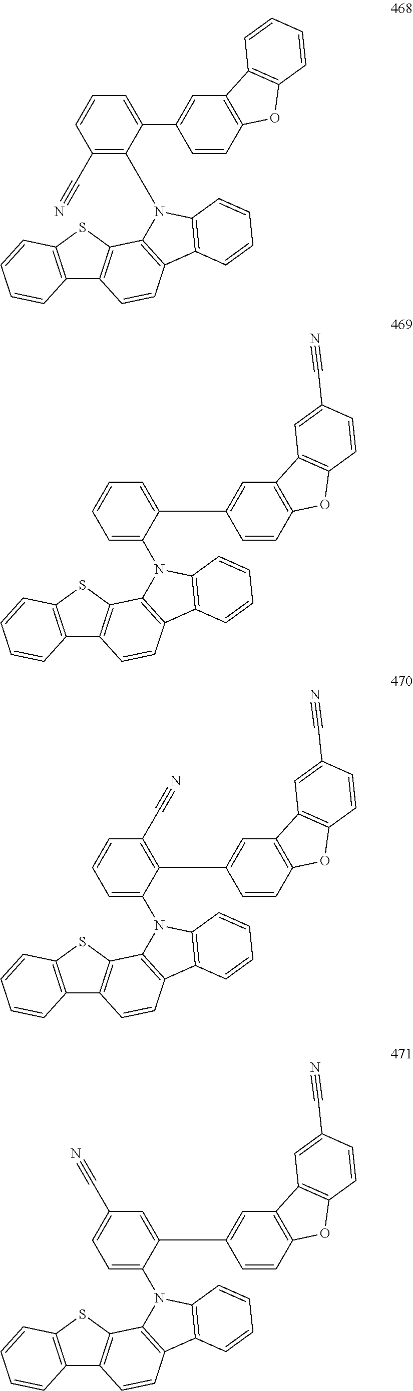 [SMILES: N#Cc1cccc(-c2ccc3oc4ccccc4c3c2)c1Cn1c2ccccc2c2ccc3c4ccccc4sc3c21 ; N#Cc1ccc2oc3ccc(Cc4ccccc4-n4c5ccccc5c5ccc6c7ccccc7sc6c54)cc3c2c1 ; N#Cc1ccc2oc3ccc(Cc4c(C#N)cccc4-n4c5ccccc5c5ccc6c7ccccc7sc6c54)cc3c2c1 ; N#Cc1ccc(-n2c3ccccc3c3ccc4c5ccccc5sc4c32)c(Cc2ccc3oc4ccc(C#N)cc4c3c2)c1]

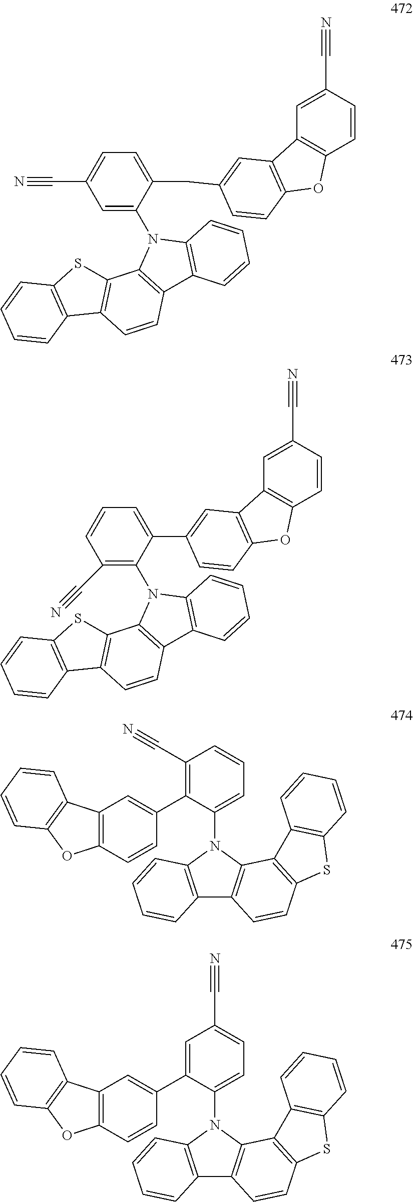

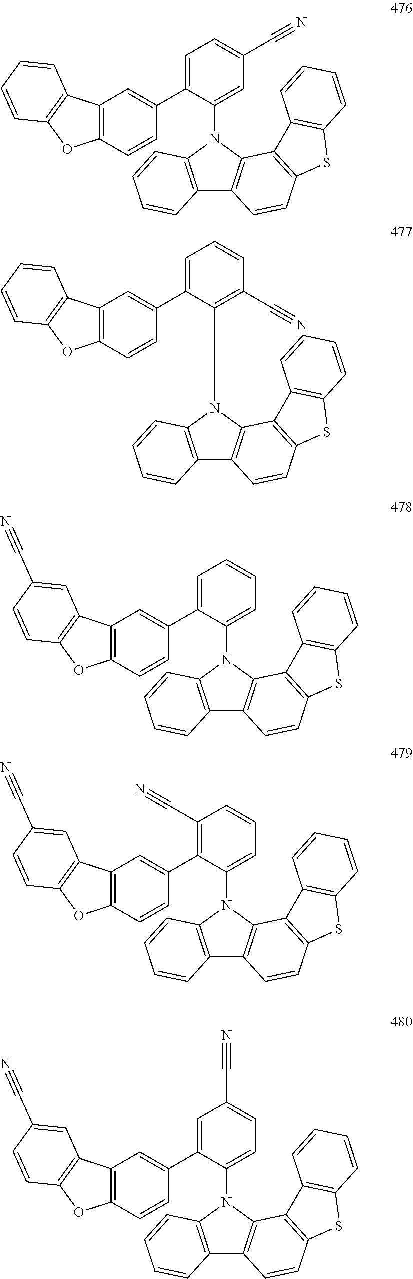

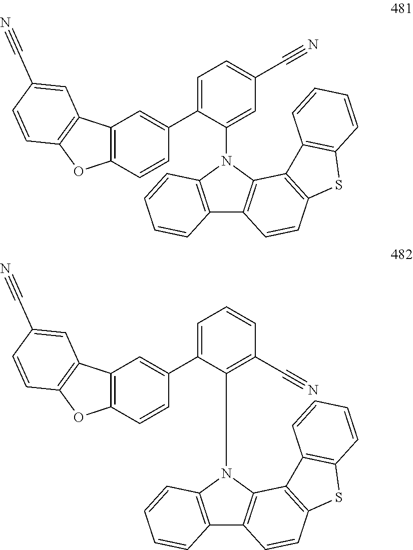 [SMILES: N#Cc1ccc(-c2ccc3oc4ccc(C#N)cc4c3c2)c(-n2c3ccccc3c3ccc4sc5ccccc5c4c32)c1 ; N#Cc1ccc2oc3ccc(-c4cccc(C#N)c4CCn4c5ccccc5c5ccc6sc7ccccc7c6c54)cc3c2c1]

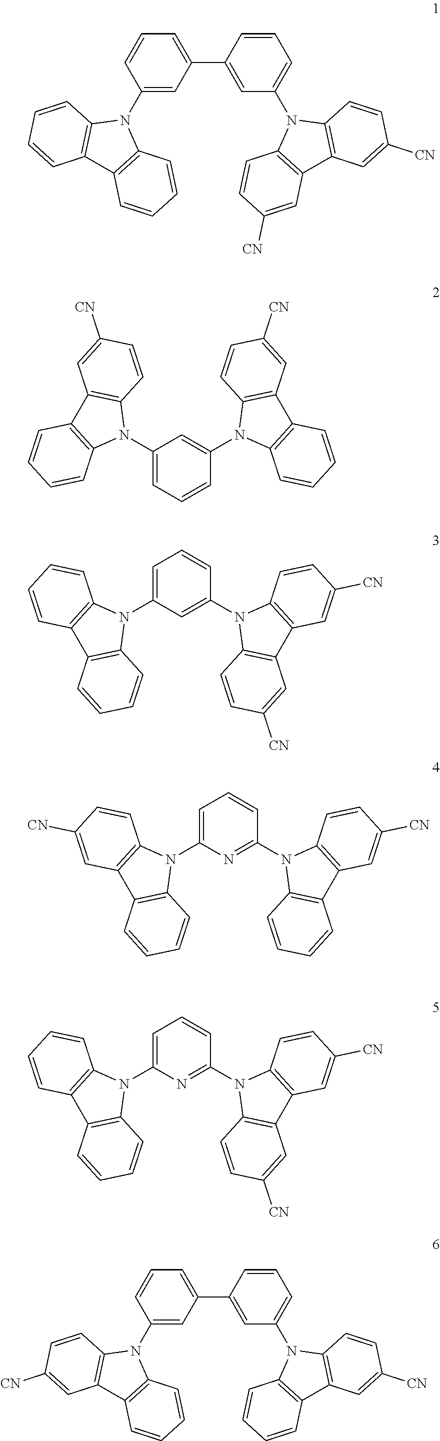

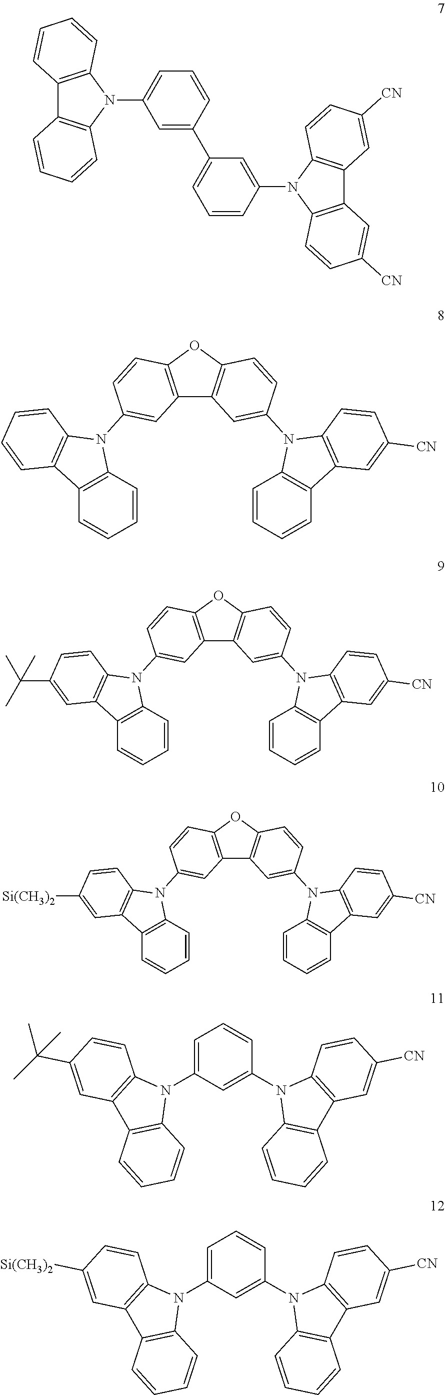

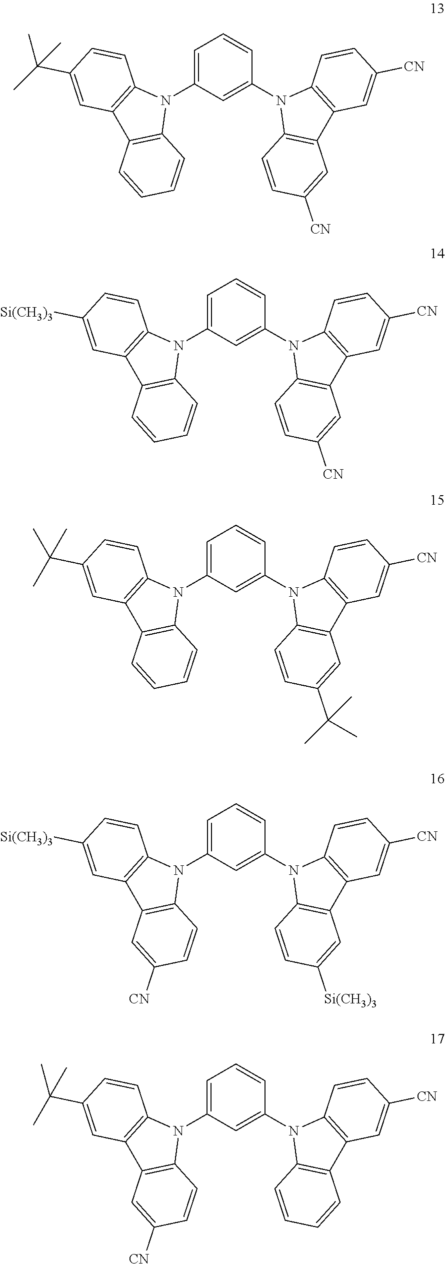

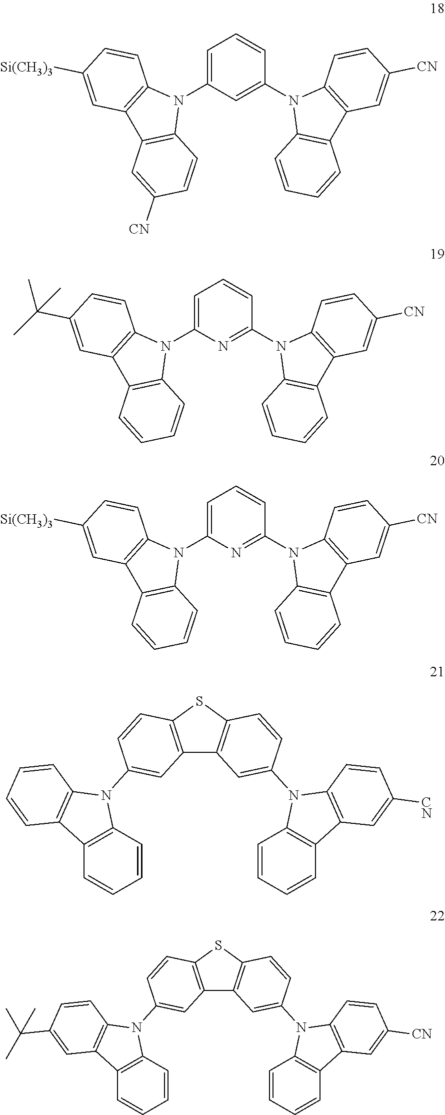

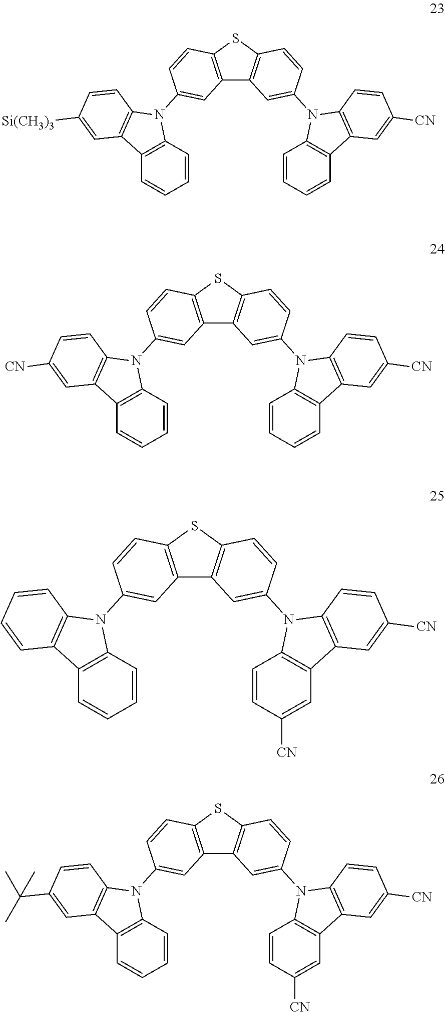

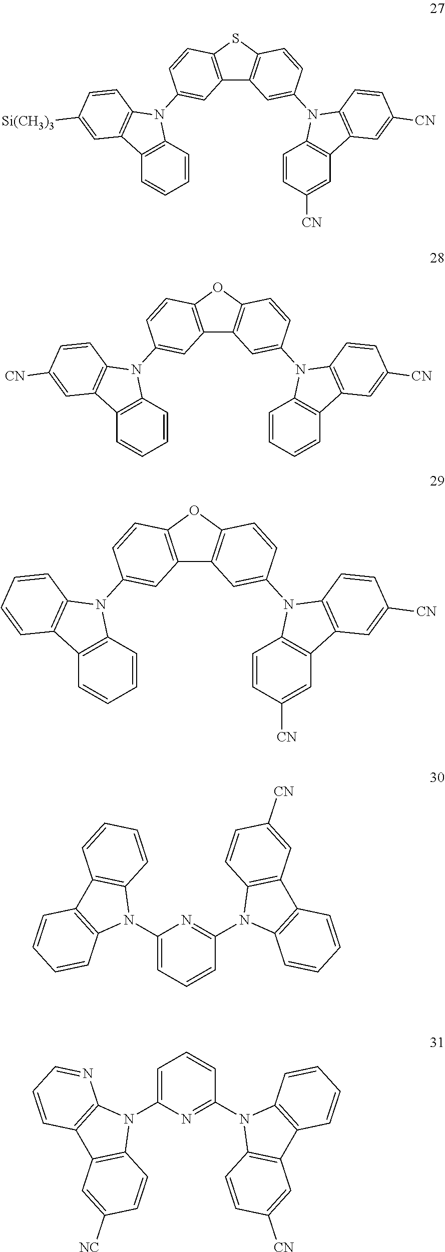

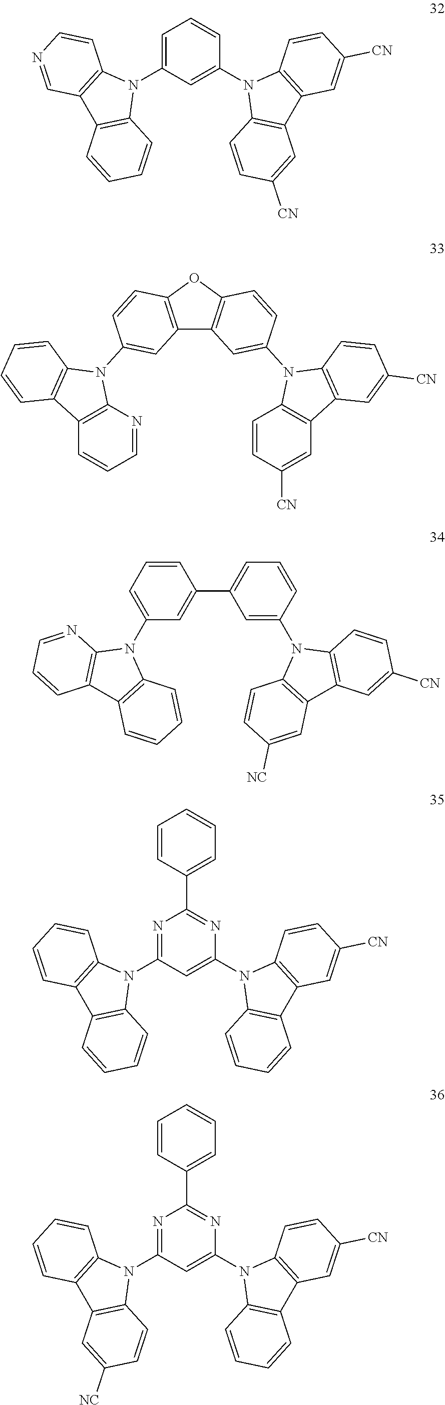 [SMILES: N#Cc1ccc2c(c1)c1cc(C#N)ccc1n2-c1cccc(-n2c3ccccc3c3cnccc32)c1 ; N#Cc1ccc2c(c1)c1cc(C#N)ccc1n2-c1ccc2oc3ccc(-n4c5ccccc5c5cccnc54)cc3c2c1 ; N#Cc1ccc2c(c1)c1cc(C#N)ccc1n2-c1cccc(-c2cccc(-n3c4ccccc4c4cccnc43)c2)c1 ; N#Cc1ccc2c(c1)c1ccccc1n2-c1cc(-n2c3ccccc3c3ccccc32)nc(-c2ccccc2)n1 ; N#Cc1ccc2c(c1)c1ccccc1n2-c1cc(-n2c3ccccc3c3cc(C#N)ccc32)nc(-c2ccccc2)n1]

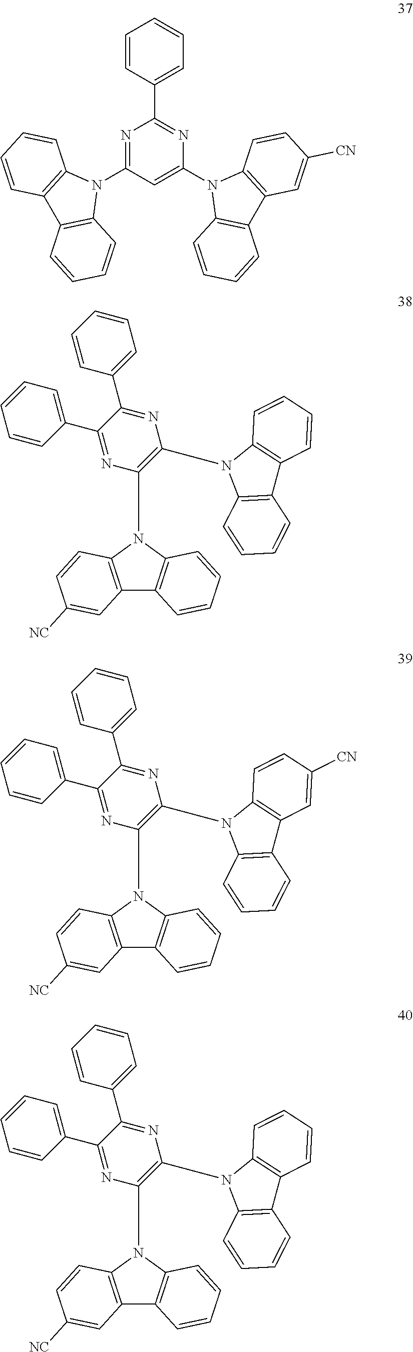

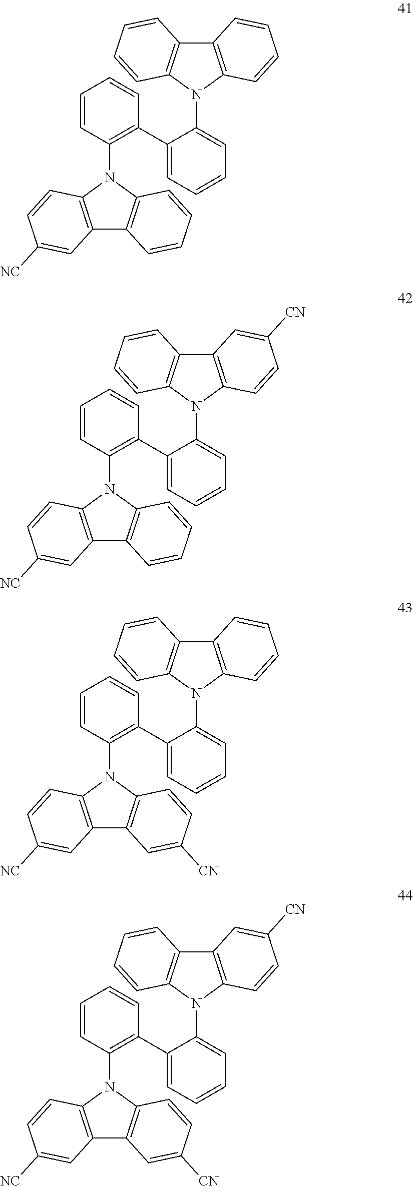 [SMILES: N#Cc1ccc2c(c1)c1ccccc1n2-c1ccccc1-c1ccccc1-n1c2ccccc2c2ccccc21 ; N#Cc1ccc2c(c1)c1ccccc1n2-c1ccccc1-c1ccccc1-n1c2ccccc2c2cc(C#N)ccc21 ; N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21]

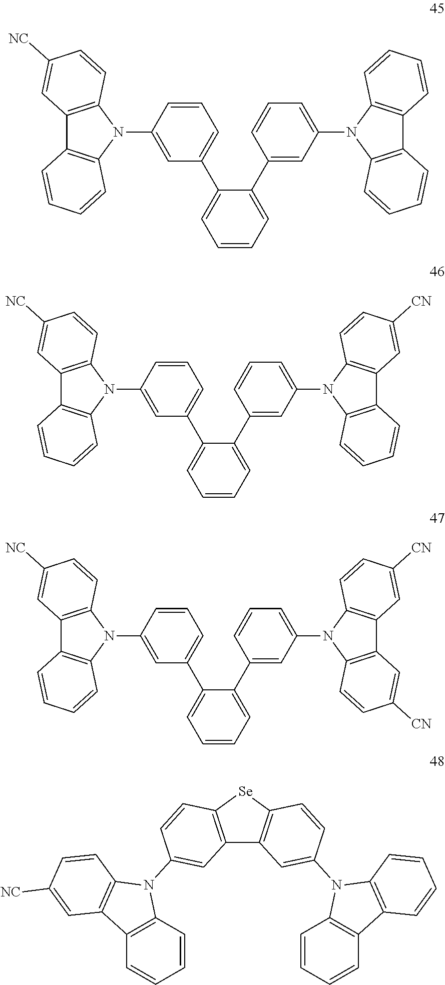 [SMILES: N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1 ; N#Cc1ccc2c(c1)c1ccccc1n2-c1ccc2[se]c3ccc(-n4c5ccccc5c5ccccc54)cc3c2c1]

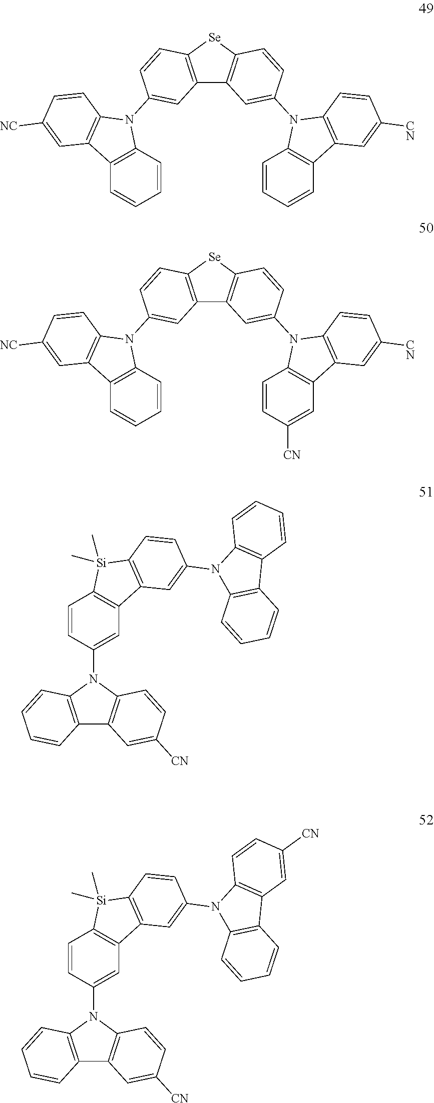 [SMILES: N#Cc1ccc2c(c1)c1ccccc1n2-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccc(C#N)cc5c5cc(C#N)ccc54)cc3c2c1 ; C[Si]1(C)c2ccc(-n3c4ccccc4c4ccccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccccc4c4cc(C#N)ccc43)ccc21]

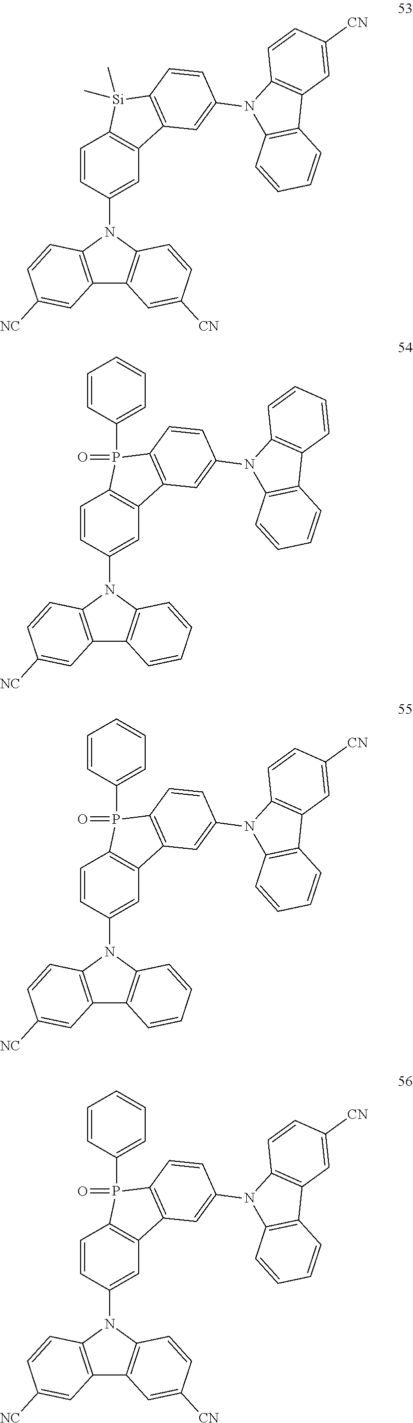 [SMILES: C[Si]1(C)c2ccc(-n3c4ccccc4c4cc(C#N)ccc43)cc2-c2cc(-n3c4ccc(C#N)cc4c4cc(C#N)ccc43)ccc21 ; N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1 ; N#Cc1ccc2c(c1)c1ccccc1n2-c1ccc2c(c1)-c1cc(-n3c4ccc(C#N)cc4c4cc(C#N)ccc43)ccc1P2(=O)c1ccccc1]

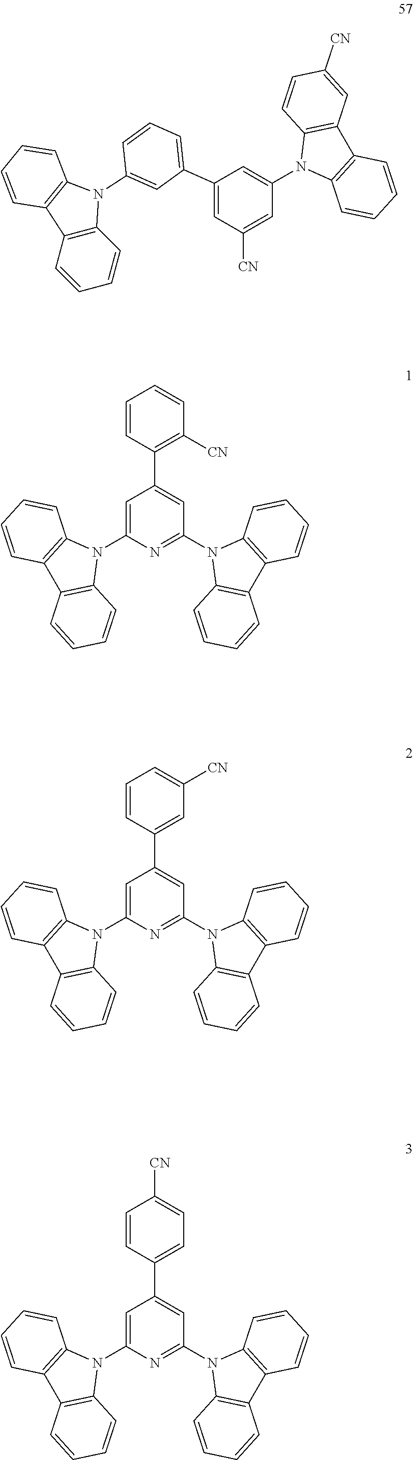 [SMILES: N#Cc1cc(-c2cccc(-n3c4ccccc4c4ccccc43)c2)cc(-n2c3ccccc3c3cc(C#N)ccc32)c1 ; N#Cc1ccccc1-c1cc(-n2c3ccccc3c3ccccc32)nc(-n2c3ccccc3c3ccccc32)c1 ; N#Cc1cccc(-c2cc(-n3c4ccccc4c4ccccc43)nc(-n3c4ccccc4c4ccccc43)c2)c1 ; N#Cc1ccc(-c2cc(-n3c4ccccc4c4ccccc43)nc(-n3c4ccccc4c4ccccc43)c2)cc1]

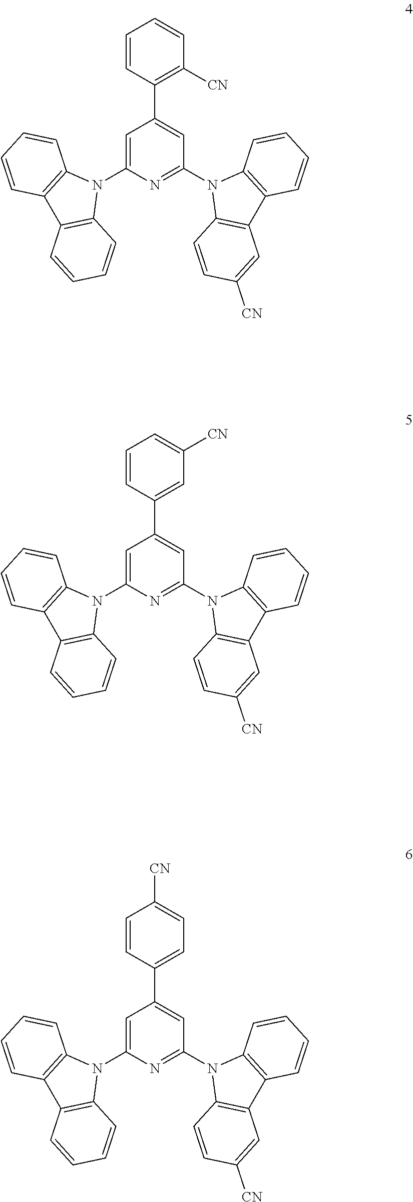 [SMILES: N#Cc1ccc2c(c1)c1ccccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccccc4c4cc(C#N)ccc43)c2)c1 ; N#Cc1ccc(-c2cc(-n3c4ccccc4c4ccccc43)nc(-n3c4ccccc4c4cc(C#N)ccc43)c2)cc1]

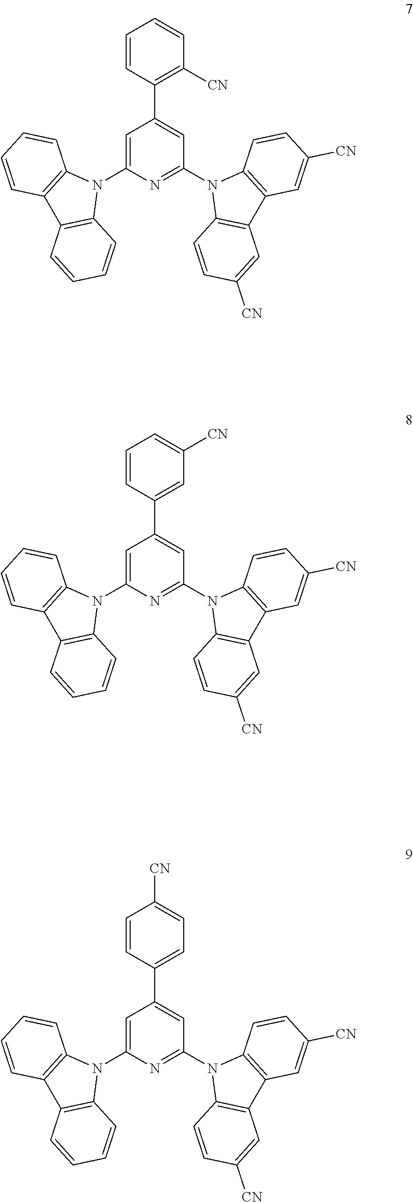 [SMILES: N#Cc1ccc2c(c1)c1cc(C#N)ccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccc(C#N)cc4c4cc(C#N)ccc43)c2)c1 ; N#Cc1ccc(-c2cc(-n3c4ccccc4c4ccccc43)nc(-n3c4ccc(C#N)cc4c4cc(C#N)ccc43)c2)cc1]

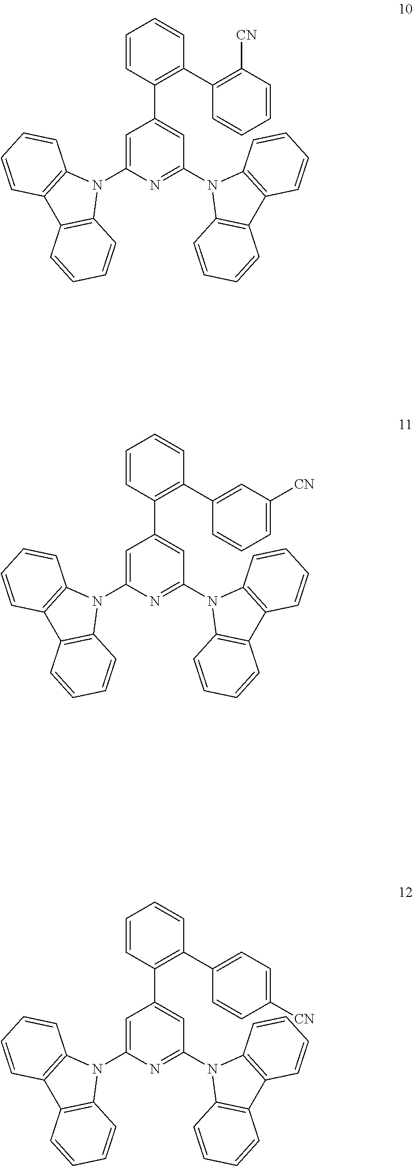 [SMILES: N#Cc1ccccc1-c1ccccc1-c1cc(-n2c3ccccc3c3ccccc32)nc(-n2c3ccccc3c3ccccc32)c1 ; N#Cc1cccc(-c2ccccc2-c2cc(-n3c4ccccc4c4ccccc43)nc(-n3c4ccccc4c4ccccc43)c2)c1 ; N#Cc1ccc(-c2ccccc2-c2cc(-n3c4ccccc4c4ccccc43)nc(-n3c4ccccc4c4ccccc43)c2)cc1]

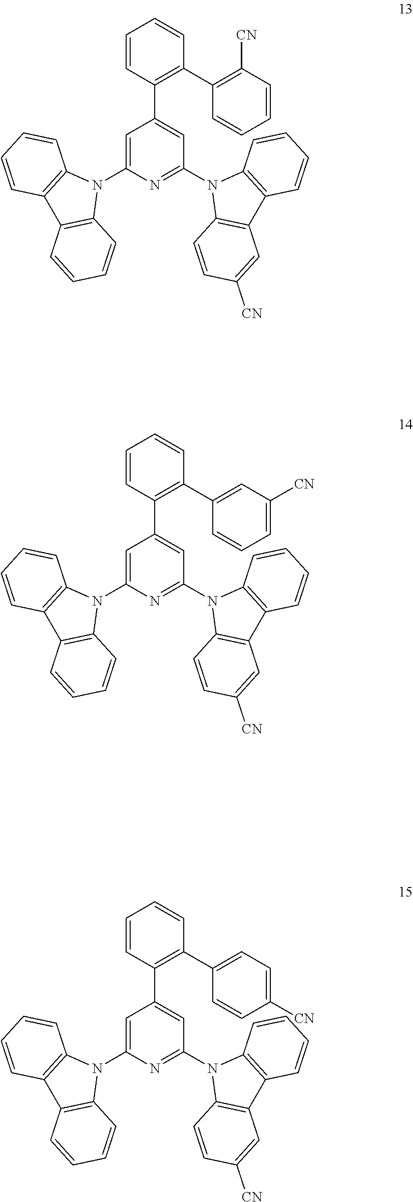 [SMILES: N#Cc1ccc2c(c1)c1ccccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccccc4c4cc(C#N)ccc43)c2)c1 ; N#Cc1ccc(-c2ccccc2-c2cc(-n3c4ccccc4c4ccccc43)nc(-n3c4ccccc4c4cc(C#N)ccc43)c2)cc1]

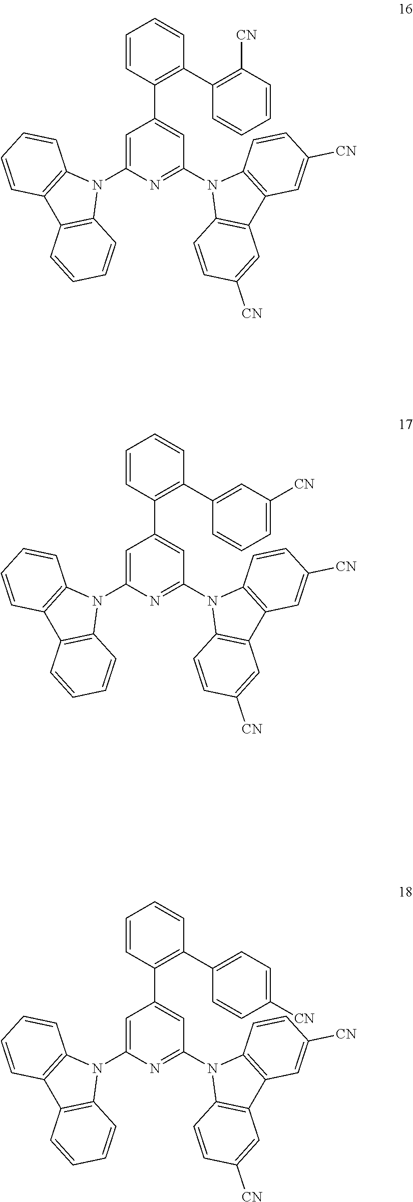 [SMILES: N#Cc1ccc2c(c1)c1cc(C#N)ccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2cc(-n3c4ccccc4c4ccccc43)nc(-n3c4ccc(C#N)cc4c4cc(C#N)ccc43)c2)cc1]

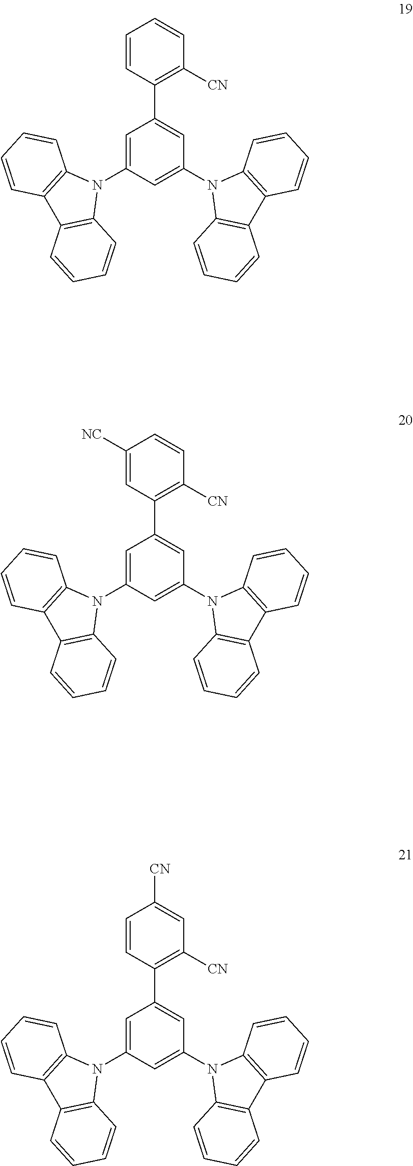 [SMILES: N#Cc1ccccc1-c1cc(-n2c3ccccc3c3ccccc32)cc(-n2c3ccccc3c3ccccc32)c1 ; N#Cc1ccc(C#N)c(-c2cc(-n3c4ccccc4c4ccccc43)cc(-n3c4ccccc4c4ccccc43)c2)c1 ; N#Cc1ccc(-c2cc(-n3c4ccccc4c4ccccc43)cc(-n3c4ccccc4c4ccccc43)c2)c(C#N)c1]

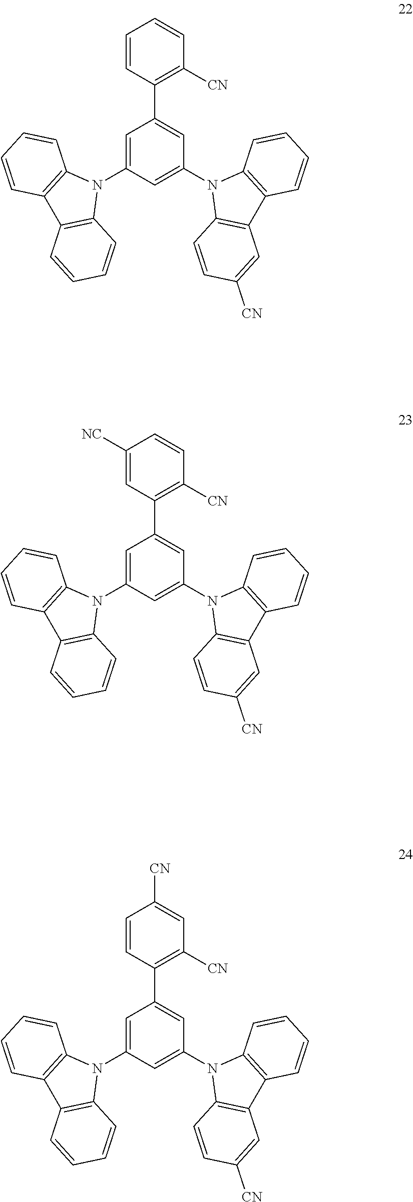 [SMILES: N#Cc1ccc2c(c1)c1ccccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccccc4c4cc(C#N)ccc43)c2)c1 ; N#Cc1ccc(-c2cc(-n3c4ccccc4c4ccccc43)cc(-n3c4ccccc4c4cc(C#N)ccc43)c2)c(C#N)c1]

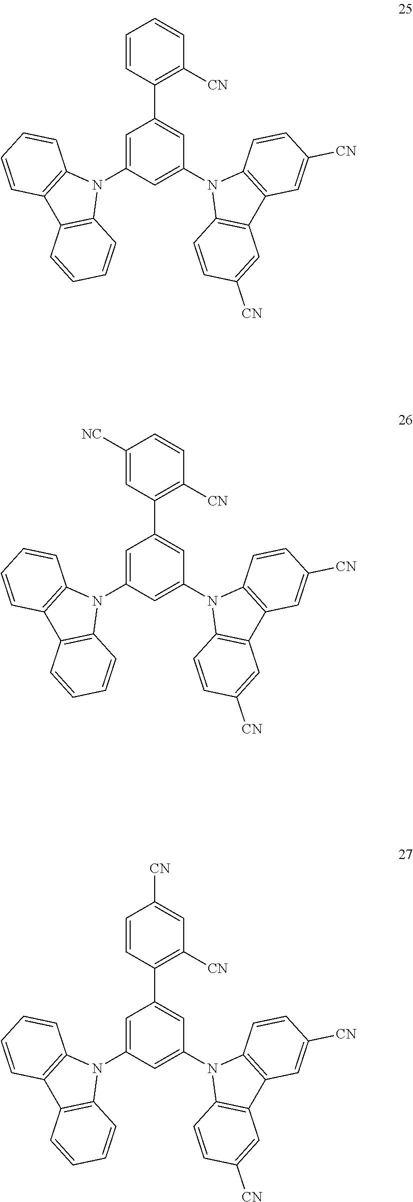 [SMILES: N#Cc1ccc2c(c1)c1cc(C#N)ccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccc(C#N)cc4c4cc(C#N)ccc43)c2)c1 ; N#Cc1ccc(-c2cc(-n3c4ccccc4c4ccccc43)cc(-n3c4ccc(C#N)cc4c4cc(C#N)ccc43)c2)c(C#N)c1]

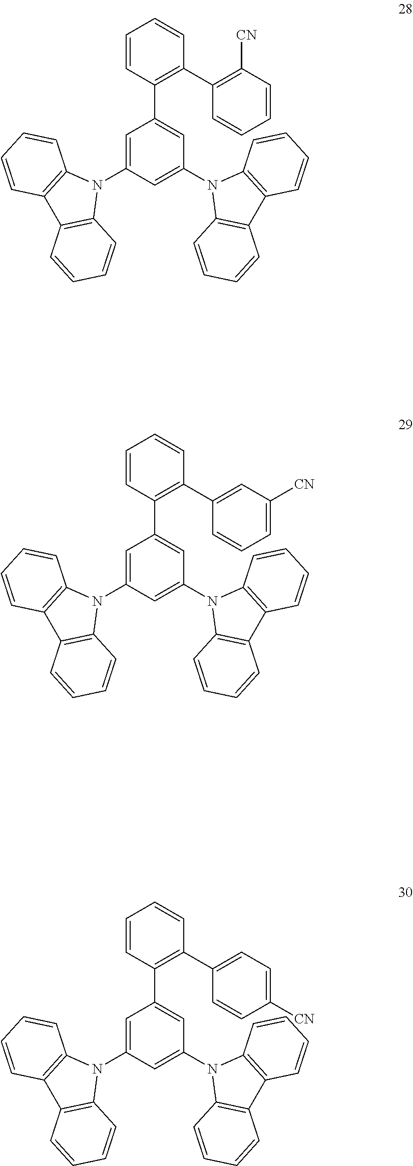 [SMILES: N#Cc1ccccc1-c1ccccc1-c1cc(-n2c3ccccc3c3ccccc32)cc(-n2c3ccccc3c3ccccc32)c1 ; N#Cc1cccc(-c2ccccc2-c2cc(-n3c4ccccc4c4ccccc43)cc(-n3c4ccccc4c4ccccc43)c2)c1 ; N#Cc1ccc(-c2ccccc2-c2cc(-n3c4ccccc4c4ccccc43)cc(-n3c4ccccc4c4ccccc43)c2)cc1]

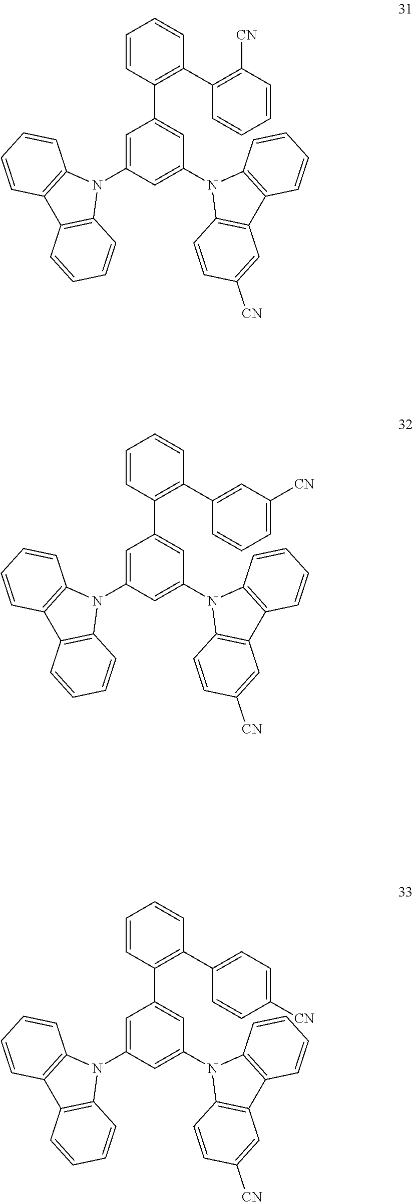 [SMILES: N#Cc1ccc2c(c1)c1ccccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccccc4c4cc(C#N)ccc43)c2)c1 ; N#Cc1ccc(-c2ccccc2-c2cc(-n3c4ccccc4c4ccccc43)cc(-n3c4ccccc4c4cc(C#N)ccc43)c2)cc1]

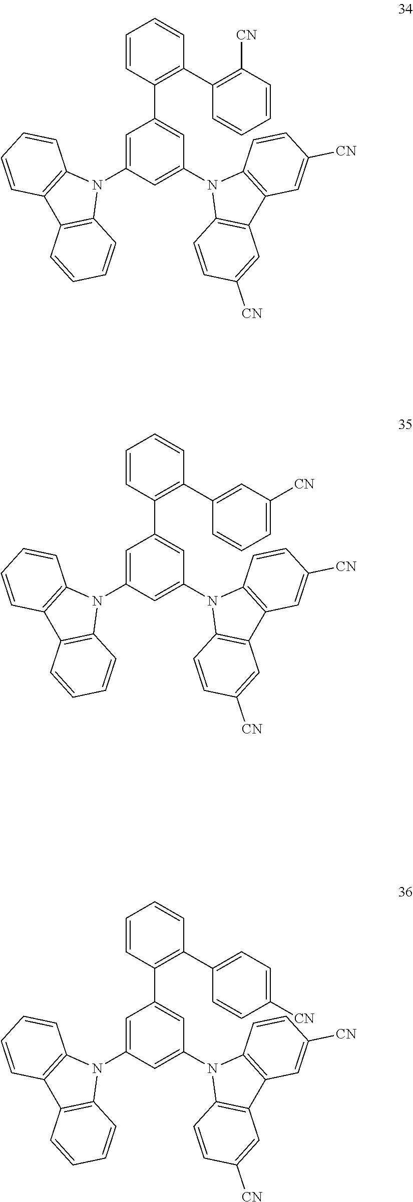 [SMILES: N#Cc1ccc2c(c1)c1cc(C#N)ccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2cc(-n3c4ccccc4c4ccccc43)cc(-n3c4ccc(C#N)cc4c4cc(C#N)ccc43)c2)cc1]

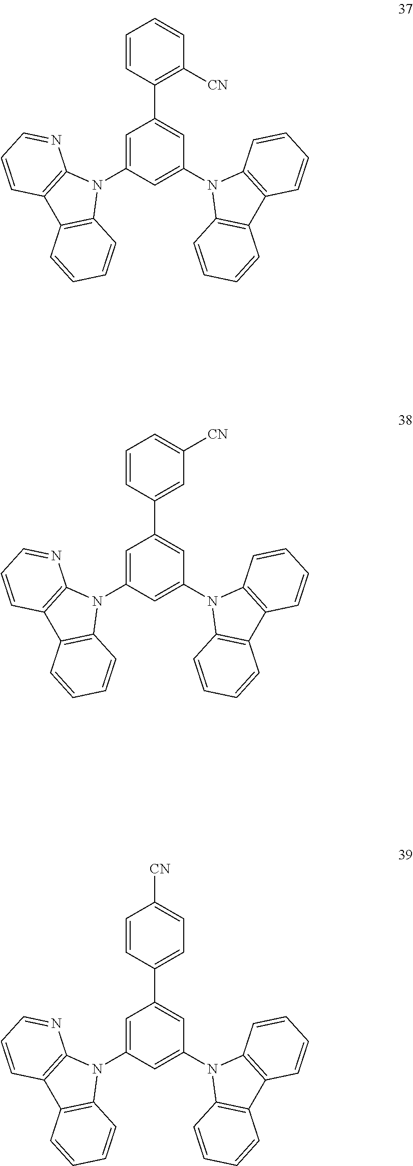 [SMILES: N#Cc1ccccc1-c1cc(-n2c3ccccc3c3ccccc32)cc(-n2c3ccccc3c3cccnc32)c1 ; N#Cc1cccc(-c2cc(-n3c4ccccc4c4ccccc43)cc(-n3c4ccccc4c4cccnc43)c2)c1 ; N#Cc1ccc(-c2cc(-n3c4ccccc4c4ccccc43)cc(-n3c4ccccc4c4cccnc43)c2)cc1]

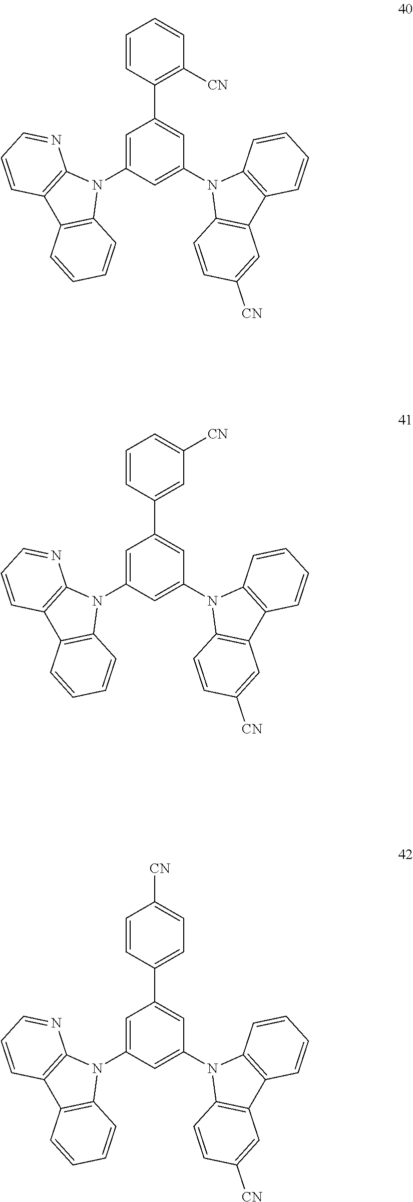 [SMILES: N#Cc1ccc2c(c1)c1ccccc1n2-c1cc(-c2ccccc2C#N)cc(-n2c3ccccc3c3cccnc32)c1 ; N#Cc1cccc(-c2cc(-n3c4ccccc4c4cc(C#N)ccc43)cc(-n3c4ccccc4c4cccnc43)c2)c1 ; N#Cc1ccc(-c2cc(-n3c4ccccc4c4cc(C#N)ccc43)cc(-n3c4ccccc4c4cccnc43)c2)cc1]

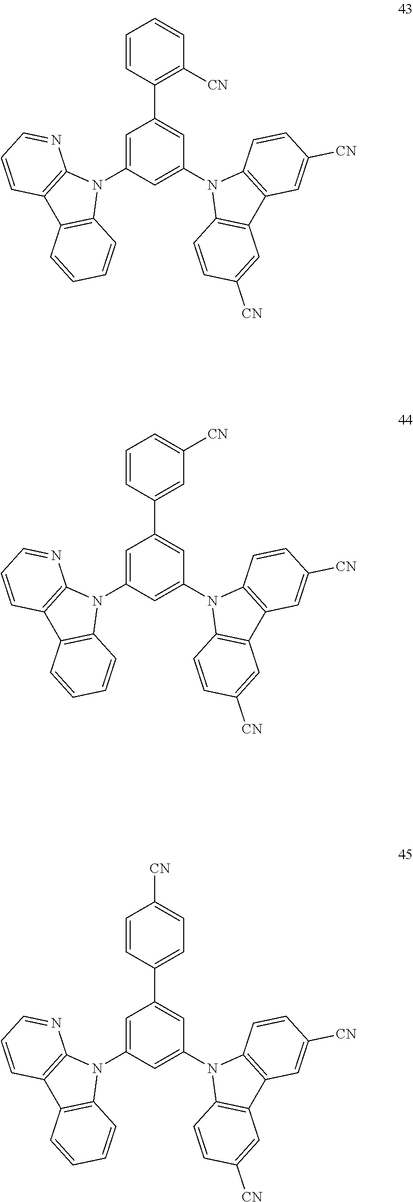 [SMILES: N#Cc1ccc2c(c1)c1cc(C#N)ccc1n2-c1cc(-c2ccccc2C#N)cc(-n2c3ccccc3c3cccnc32)c1 ; N#Cc1cccc(-c2cc(-n3c4ccc(C#N)cc4c4cc(C#N)ccc43)cc(-n3c4ccccc4c4cccnc43)c2)c1 ; N#Cc1ccc(-c2cc(-n3c4ccc(C#N)cc4c4cc(C#N)ccc43)cc(-n3c4ccccc4c4cccnc43)c2)cc1]

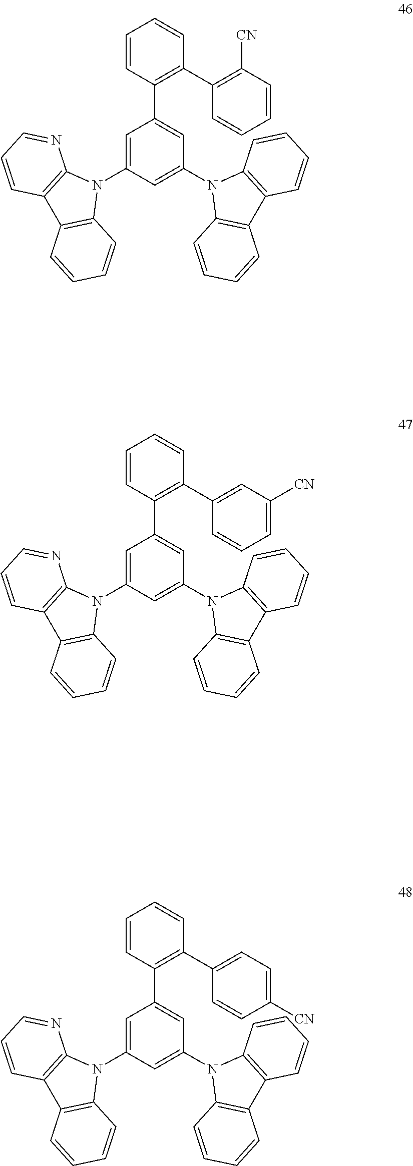 [SMILES: N#Cc1ccccc1-c1ccccc1-c1cc(-n2c3ccccc3c3ccccc32)cc(-n2c3ccccc3c3cccnc32)c1 ; N#Cc1cccc(-c2ccccc2-c2cc(-n3c4ccccc4c4ccccc43)cc(-n3c4ccccc4c4cccnc43)c2)c1 ; N#Cc1ccc(-c2ccccc2-c2cc(-n3c4ccccc4c4ccccc43)cc(-n3c4ccccc4c4cccnc43)c2)cc1]

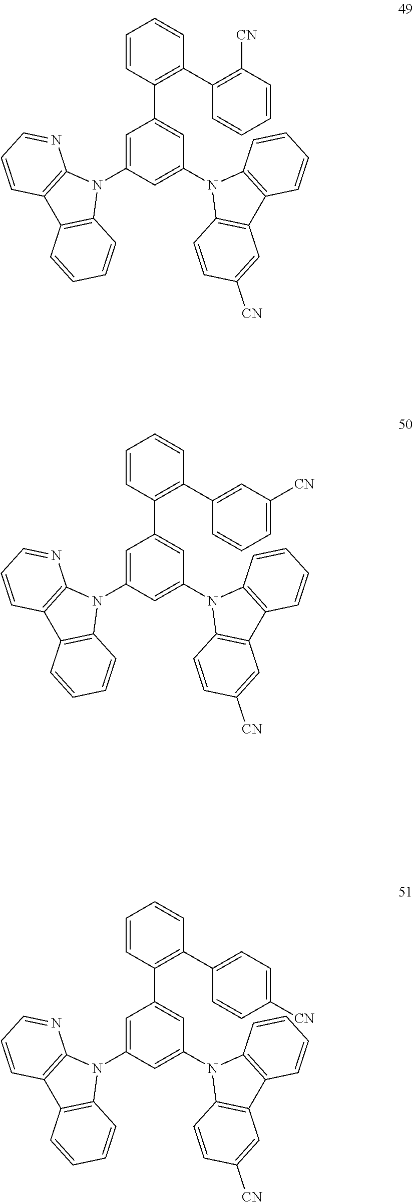 [SMILES: N#Cc1ccc2c(c1)c1ccccc1n2-c1cc(-c2ccccc2-c2ccccc2C#N)cc(-n2c3ccccc3c3cccnc32)c1 ; N#Cc1cccc(-c2ccccc2-c2cc(-n3c4ccccc4c4cc(C#N)ccc43)cc(-n3c4ccccc4c4cccnc43)c2)c1 ; N#Cc1ccc(-c2ccccc2-c2cc(-n3c4ccccc4c4cc(C#N)ccc43)cc(-n3c4ccccc4c4cccnc43)c2)cc1]

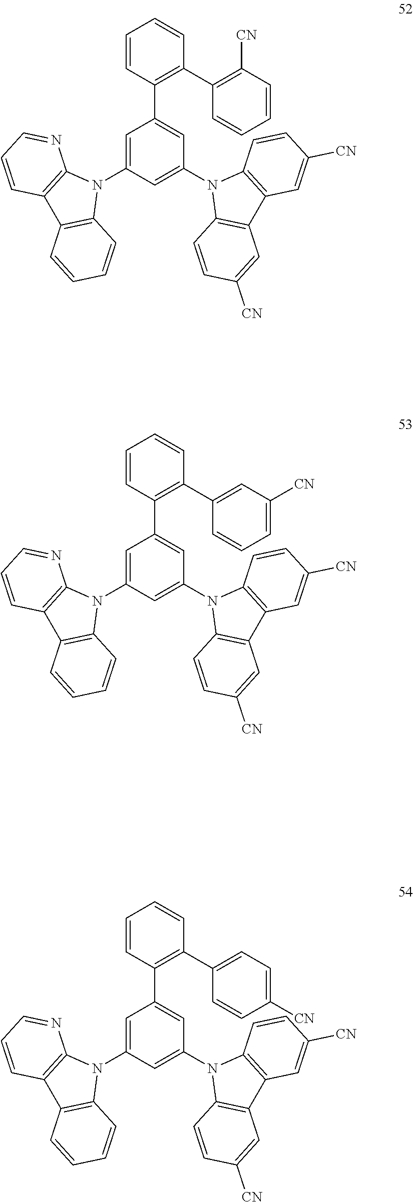 [SMILES: N#Cc1ccc2c(c1)c1cc(C#N)ccc1n2-c1cc(-c2ccccc2-c2ccccc2C#N)cc(-n2c3ccccc3c3cccnc32)c1 ; N#Cc1cccc(-c2ccccc2-c2cc(-n3c4ccc(C#N)cc4c4cc(C#N)ccc43)cc(-n3c4ccccc4c4cccnc43)c2)c1 ; N#Cc1ccc(-c2ccccc2-c2cc(-n3c4ccc(C#N)cc4c4cc(C#N)ccc43)cc(-n3c4ccccc4c4cccnc43)c2)cc1]

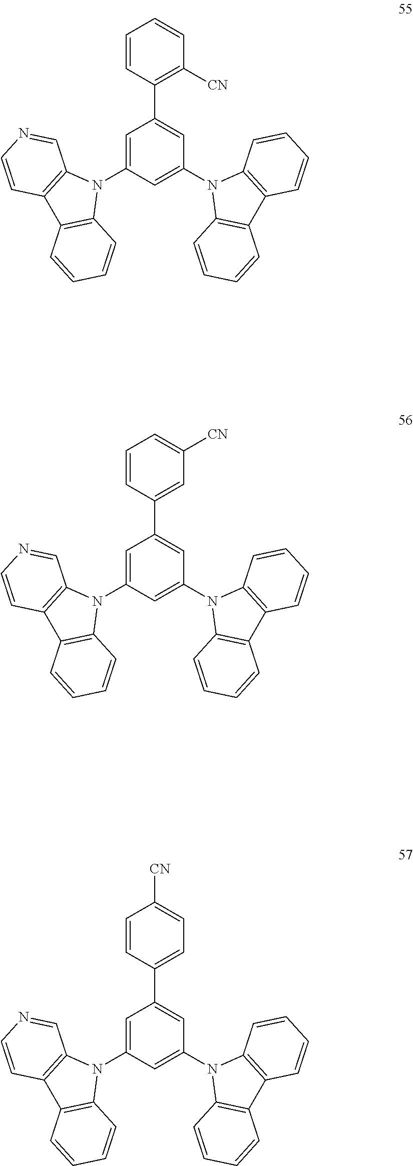 [SMILES: N#Cc1ccccc1-c1cc(-n2c3ccccc3c3ccccc32)cc(-n2c3ccccc3c3ccncc32)c1 ; N#Cc1cccc(-c2cc(-n3c4ccccc4c4ccccc43)cc(-n3c4ccccc4c4ccncc43)c2)c1 ; N#Cc1ccc(-c2cc(-n3c4ccccc4c4ccccc43)cc(-n3c4ccccc4c4ccncc43)c2)cc1]

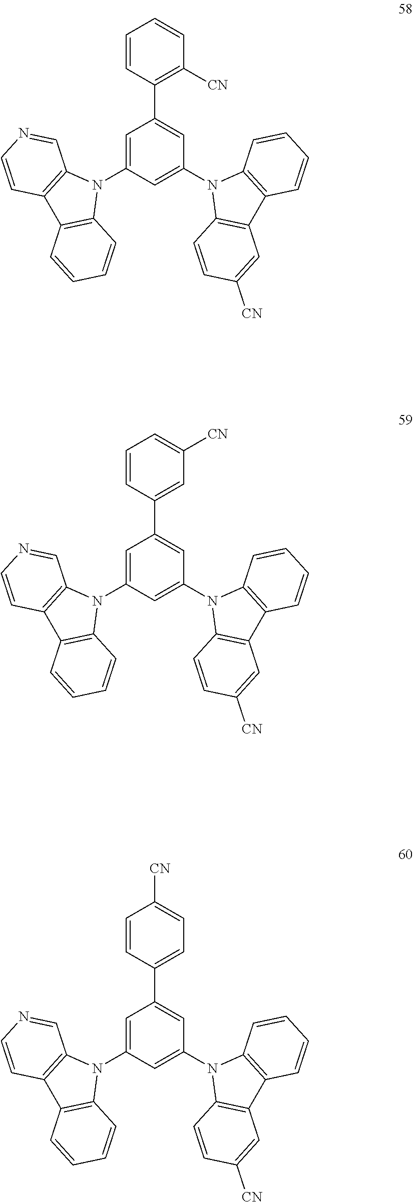 [SMILES: N#Cc1ccc2c(c1)c1ccccc1n2-c1cc(-c2ccccc2C#N)cc(-n2c3ccccc3c3ccncc32)c1 ; N#Cc1cccc(-c2cc(-n3c4ccccc4c4cc(C#N)ccc43)cc(-n3c4ccccc4c4ccncc43)c2)c1 ; N#Cc1ccc(-c2cc(-n3c4ccccc4c4cc(C#N)ccc43)cc(-n3c4ccccc4c4ccncc43)c2)cc1]

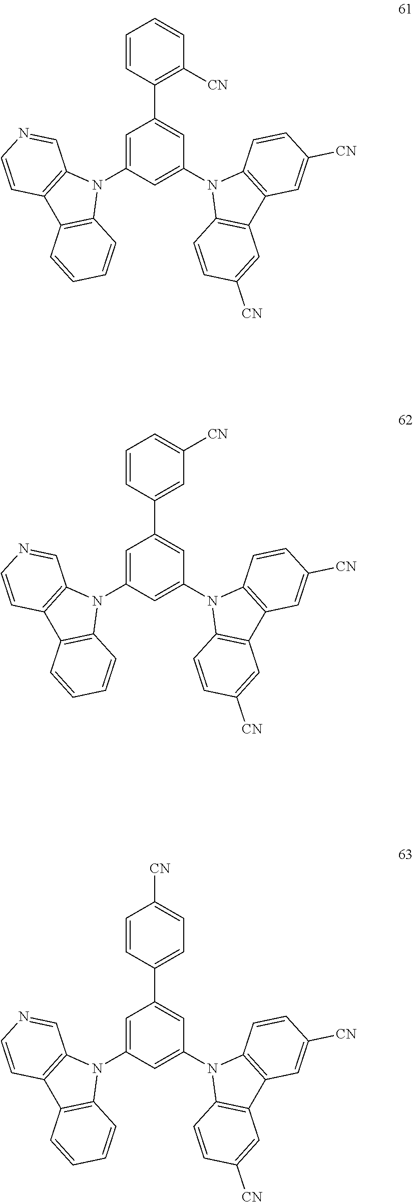 [SMILES: N#Cc1ccc2c(c1)c1cc(C#N)ccc1n2-c1cc(-c2ccccc2C#N)cc(-n2c3ccccc3c3ccncc32)c1 ; N#Cc1cccc(-c2cc(-n3c4ccc(C#N)cc4c4cc(C#N)ccc43)cc(-n3c4ccccc4c4ccncc43)c2)c1 ; N#Cc1ccc(-c2cc(-n3c4ccc(C#N)cc4c4cc(C#N)ccc43)cc(-n3c4ccccc4c4ccncc43)c2)cc1]

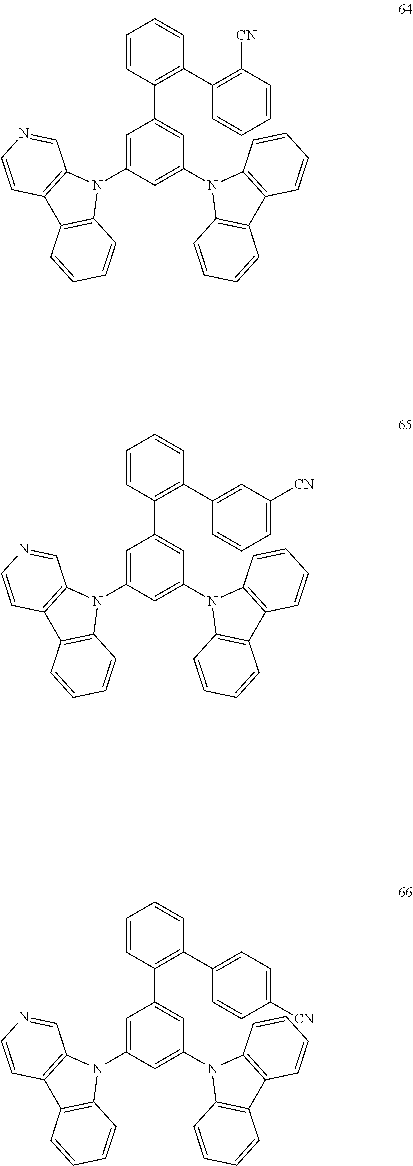 [SMILES: N#Cc1ccccc1-c1ccccc1-c1cc(-n2c3ccccc3c3ccccc32)cc(-n2c3ccccc3c3ccncc32)c1 ; N#Cc1cccc(-c2ccccc2-c2cc(-n3c4ccccc4c4ccccc43)cc(-n3c4ccccc4c4ccncc43)c2)c1 ; N#Cc1ccc(-c2ccccc2-c2cc(-n3c4ccccc4c4ccccc43)cc(-n3c4ccccc4c4ccncc43)c2)cc1]

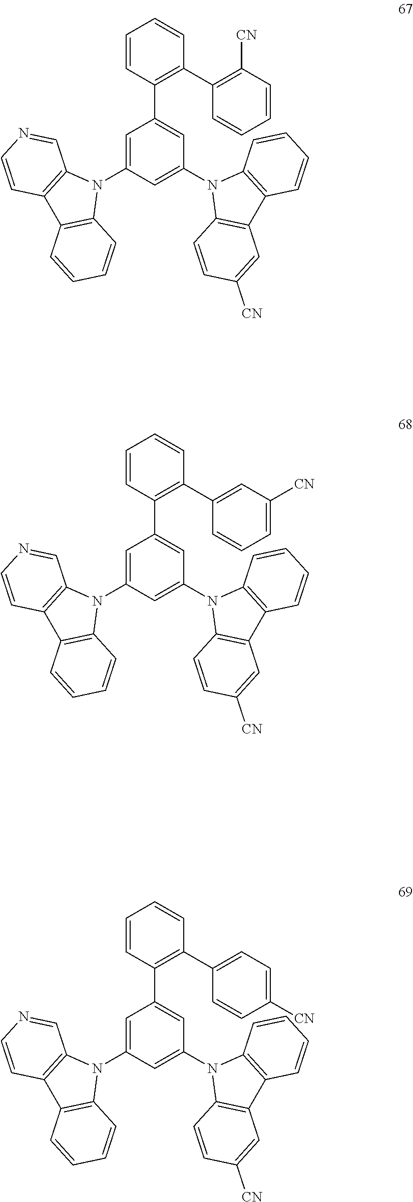 [SMILES: N#Cc1ccc2c(c1)c1ccccc1n2-c1cc(-c2ccccc2-c2ccccc2C#N)cc(-n2c3ccccc3c3ccncc32)c1 ; N#Cc1cccc(-c2ccccc2-c2cc(-n3c4ccccc4c4cc(C#N)ccc43)cc(-n3c4ccccc4c4ccncc43)c2)c1 ; N#Cc1ccc(-c2ccccc2-c2cc(-n3c4ccccc4c4cc(C#N)ccc43)cc(-n3c4ccccc4c4ccncc43)c2)cc1]

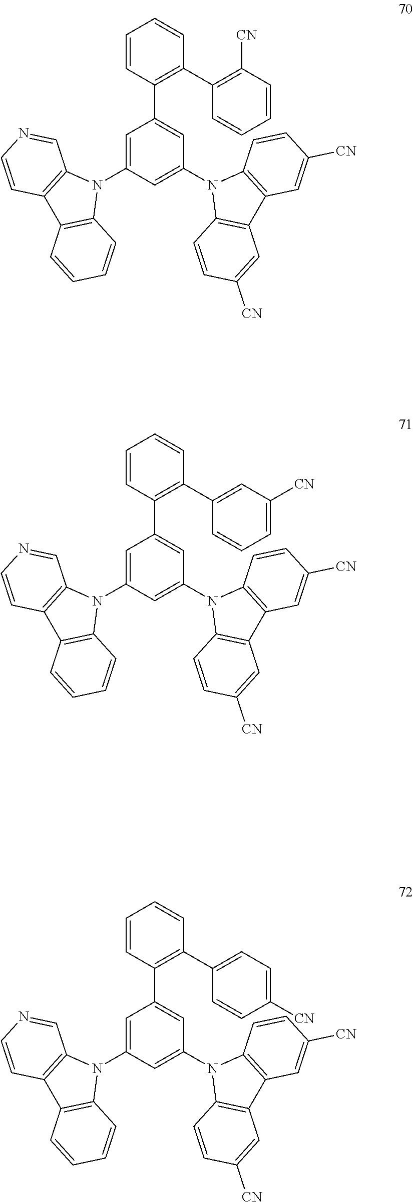 [SMILES: N#Cc1ccc2c(c1)c1cc(C#N)ccc1n2-c1cc(-c2ccccc2-c2ccccc2C#N)cc(-n2c3ccccc3c3ccncc32)c1 ; N#Cc1cccc(-c2ccccc2-c2cc(-n3c4ccc(C#N)cc4c4cc(C#N)ccc43)cc(-n3c4ccccc4c4ccncc43)c2)c1 ; N#Cc1ccc(-c2ccccc2-c2cc(-n3c4ccc(C#N)cc4c4cc(C#N)ccc43)cc(-n3c4ccccc4c4ccncc43)c2)cc1]

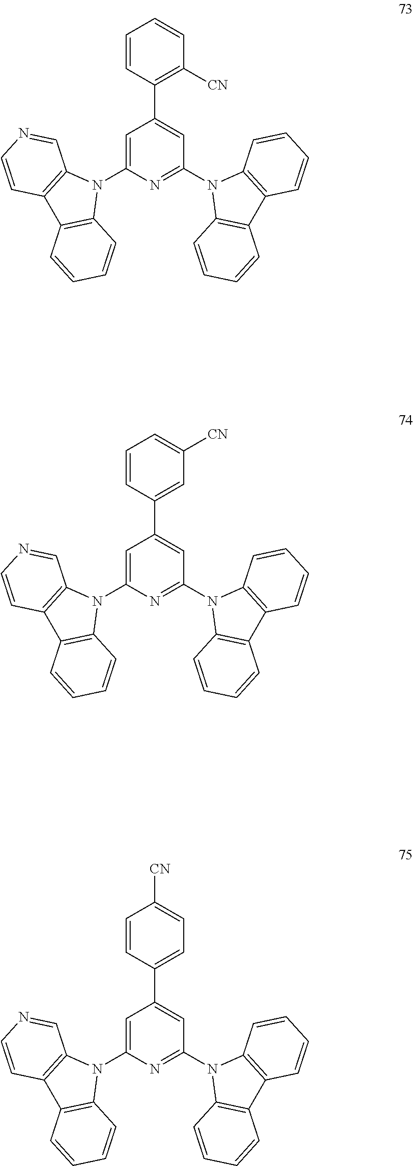 [SMILES: N#Cc1ccccc1-c1cc(-n2c3ccccc3c3ccccc32)nc(-n2c3ccccc3c3ccncc32)c1 ; N#Cc1cccc(-c2cc(-n3c4ccccc4c4ccccc43)nc(-n3c4ccccc4c4ccncc43)c2)c1 ; N#Cc1ccc(-c2cc(-n3c4ccccc4c4ccccc43)nc(-n3c4ccccc4c4ccncc43)c2)cc1]

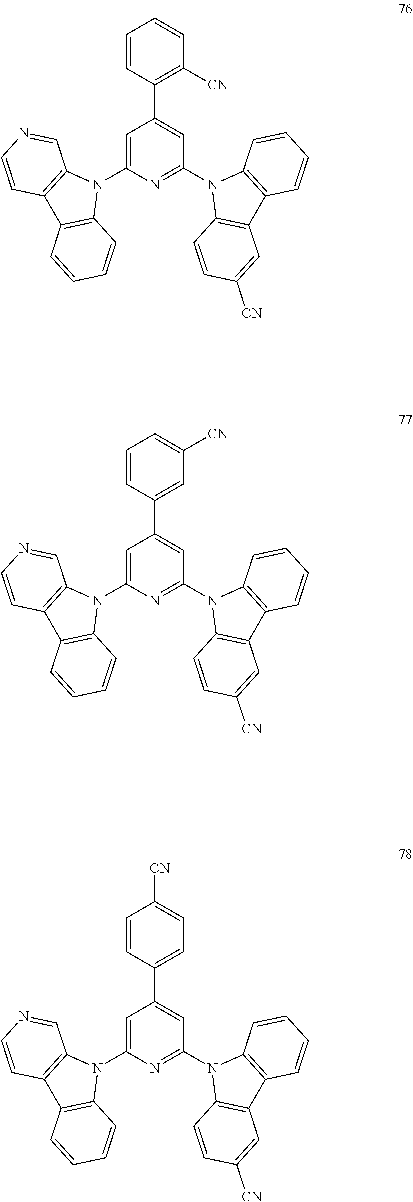 [SMILES: N#Cc1ccc2c(c1)c1ccccc1n2-c1cc(-c2ccccc2C#N)cc(-n2c3ccccc3c3ccncc32)n1 ; N#Cc1cccc(-c2cc(-n3c4ccccc4c4cc(C#N)ccc43)nc(-n3c4ccccc4c4ccncc43)c2)c1 ; N#Cc1ccc(-c2cc(-n3c4ccccc4c4cc(C#N)ccc43)nc(-n3c4ccccc4c4ccncc43)c2)cc1]

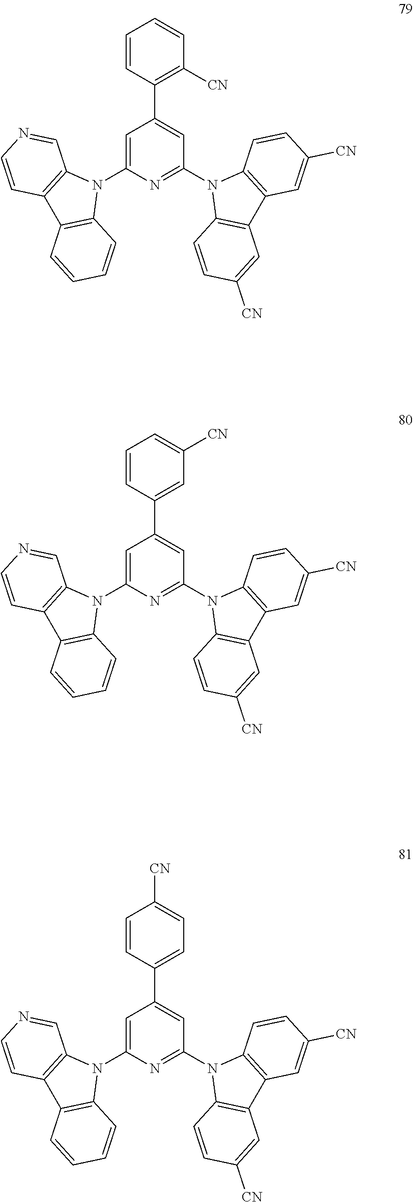 [SMILES: N#Cc1ccc2c(c1)c1cc(C#N)ccc1n2-c1cc(-c2ccccc2C#N)cc(-n2c3ccccc3c3ccncc32)n1 ; N#Cc1cccc(-c2cc(-n3c4ccc(C#N)cc4c4cc(C#N)ccc43)nc(-n3c4ccccc4c4ccncc43)c2)c1 ; N#Cc1ccc(-c2cc(-n3c4ccc(C#N)cc4c4cc(C#N)ccc43)nc(-n3c4ccccc4c4ccncc43)c2)cc1]

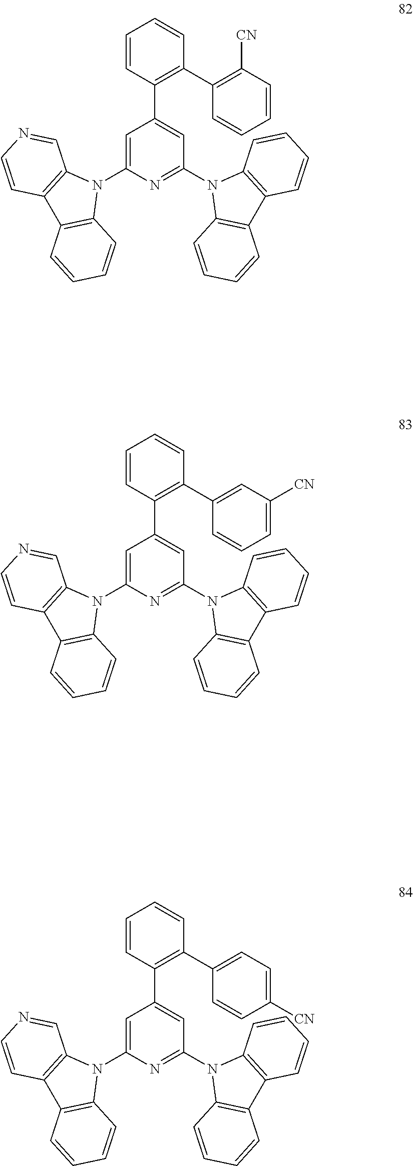 [SMILES: N#Cc1ccccc1-c1ccccc1-c1cc(-n2c3ccccc3c3ccccc32)nc(-n2c3ccccc3c3ccncc32)c1 ; N#Cc1cccc(-c2ccccc2-c2cc(-n3c4ccccc4c4ccccc43)nc(-n3c4ccccc4c4ccncc43)c2)c1 ; N#Cc1ccc(-c2ccccc2-c2cc(-n3c4ccccc4c4ccccc43)nc(-n3c4ccccc4c4ccncc43)c2)cc1]

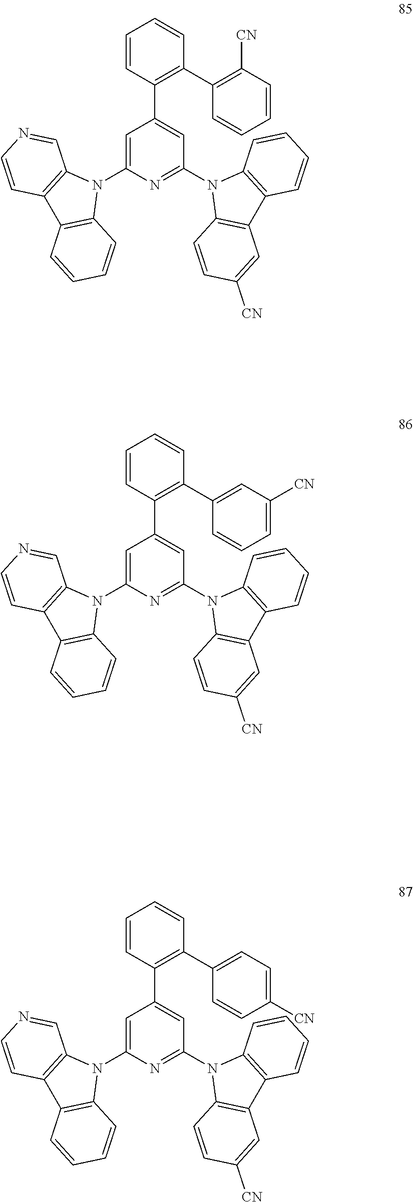 [SMILES: N#Cc1ccc2c(c1)c1ccccc1n2-c1cc(-c2ccccc2-c2ccccc2C#N)cc(-n2c3ccccc3c3ccncc32)n1 ; N#Cc1cccc(-c2ccccc2-c2cc(-n3c4ccccc4c4cc(C#N)ccc43)nc(-n3c4ccccc4c4ccncc43)c2)c1 ; N#Cc1ccc(-c2ccccc2-c2cc(-n3c4ccccc4c4cc(C#N)ccc43)nc(-n3c4ccccc4c4ccncc43)c2)cc1]

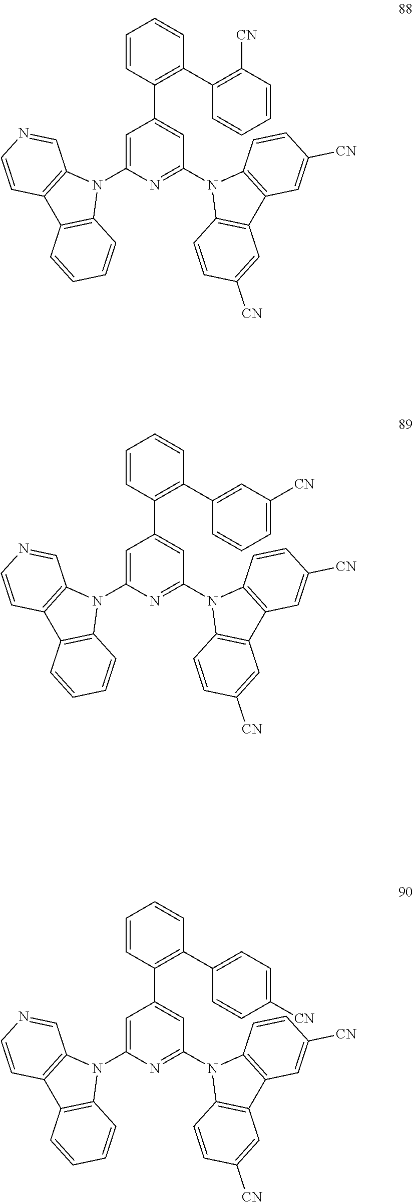 [SMILES: N#Cc1ccc2c(c1)c1cc(C#N)ccc1n2-c1cc(-c2ccccc2-c2ccccc2C#N)cc(-n2c3ccccc3c3ccncc32)n1 ; N#Cc1cccc(-c2ccccc2-c2cc(-n3c4ccc(C#N)cc4c4cc(C#N)ccc43)nc(-n3c4ccccc4c4ccncc43)c2)c1 ; N#Cc1ccc(-c2ccccc2-c2cc(-n3c4ccc(C#N)cc4c4cc(C#N)ccc43)nc(-n3c4ccccc4c4ccncc43)c2)cc1]

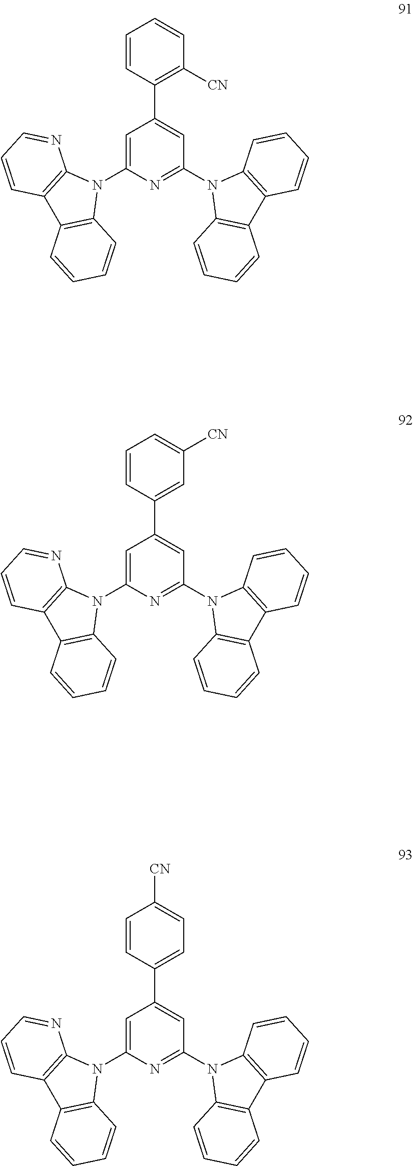 [SMILES: N#Cc1ccccc1-c1cc(-n2c3ccccc3c3ccccc32)nc(-n2c3ccccc3c3cccnc32)c1 ; N#Cc1cccc(-c2cc(-n3c4ccccc4c4ccccc43)nc(-n3c4ccccc4c4cccnc43)c2)c1 ; N#Cc1ccc(-c2cc(-n3c4ccccc4c4ccccc43)nc(-n3c4ccccc4c4cccnc43)c2)cc1]

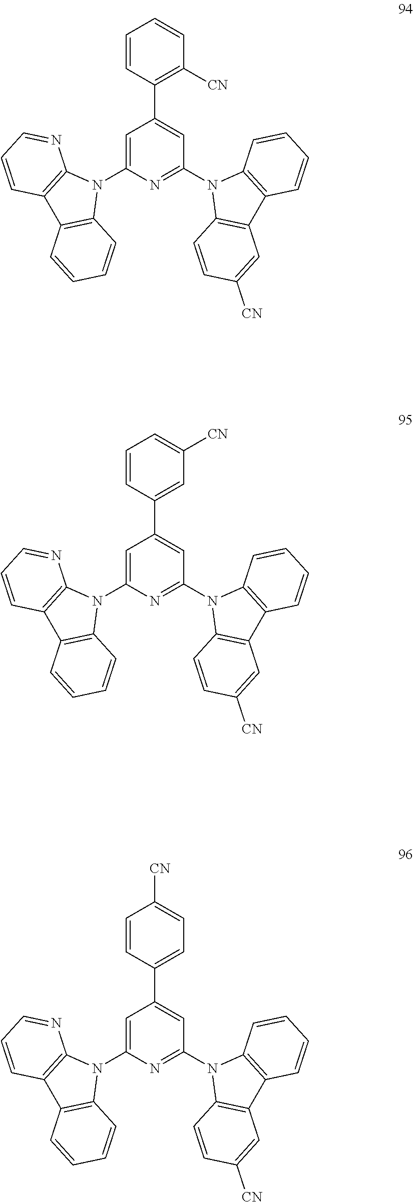 [SMILES: N#Cc1ccc2c(c1)c1ccccc1n2-c1cc(-c2ccccc2C#N)cc(-n2c3ccccc3c3cccnc32)n1 ; N#Cc1cccc(-c2cc(-n3c4ccccc4c4cc(C#N)ccc43)nc(-n3c4ccccc4c4cccnc43)c2)c1 ; N#Cc1ccc(-c2cc(-n3c4ccccc4c4cc(C#N)ccc43)nc(-n3c4ccccc4c4cccnc43)c2)cc1]

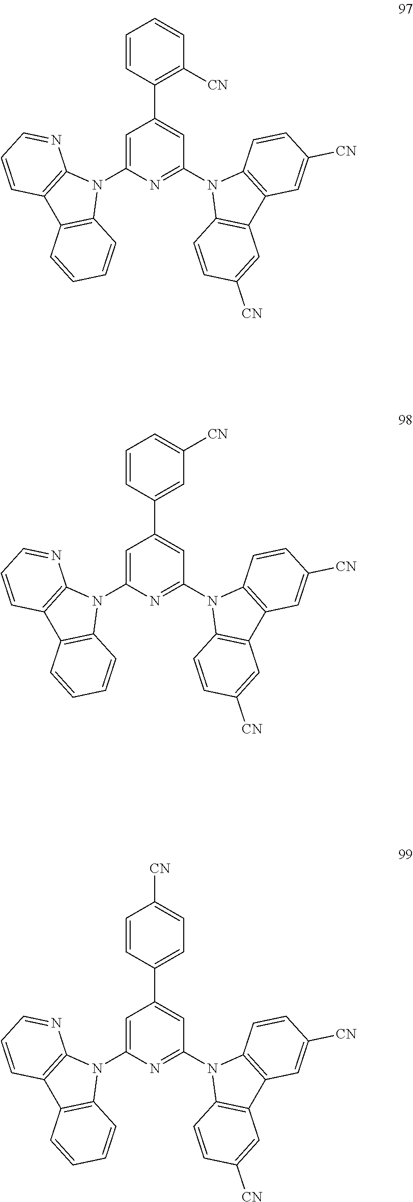 [SMILES: N#Cc1ccc2c(c1)c1cc(C#N)ccc1n2-c1cc(-c2ccccc2C#N)cc(-n2c3ccccc3c3cccnc32)n1 ; N#Cc1cccc(-c2cc(-n3c4ccc(C#N)cc4c4cc(C#N)ccc43)nc(-n3c4ccccc4c4cccnc43)c2)c1 ; N#Cc1ccc(-c2cc(-n3c4ccc(C#N)cc4c4cc(C#N)ccc43)nc(-n3c4ccccc4c4cccnc43)c2)cc1]

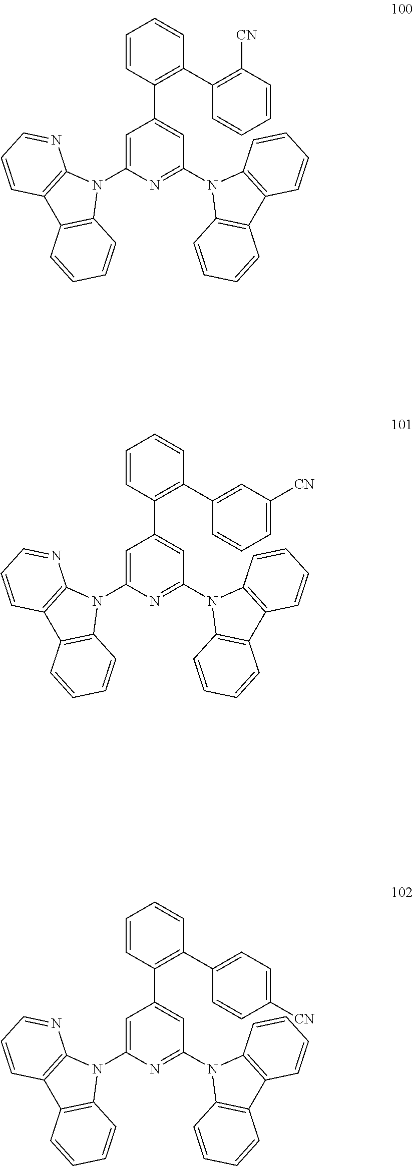 [SMILES: N#Cc1ccccc1-c1ccccc1-c1cc(-n2c3ccccc3c3ccccc32)nc(-n2c3ccccc3c3cccnc32)c1 ; N#Cc1cccc(-c2ccccc2-c2cc(-n3c4ccccc4c4ccccc43)nc(-n3c4ccccc4c4cccnc43)c2)c1 ; N#Cc1ccc(-c2ccccc2-c2cc(-n3c4ccccc4c4ccccc43)nc(-n3c4ccccc4c4cccnc43)c2)cc1]

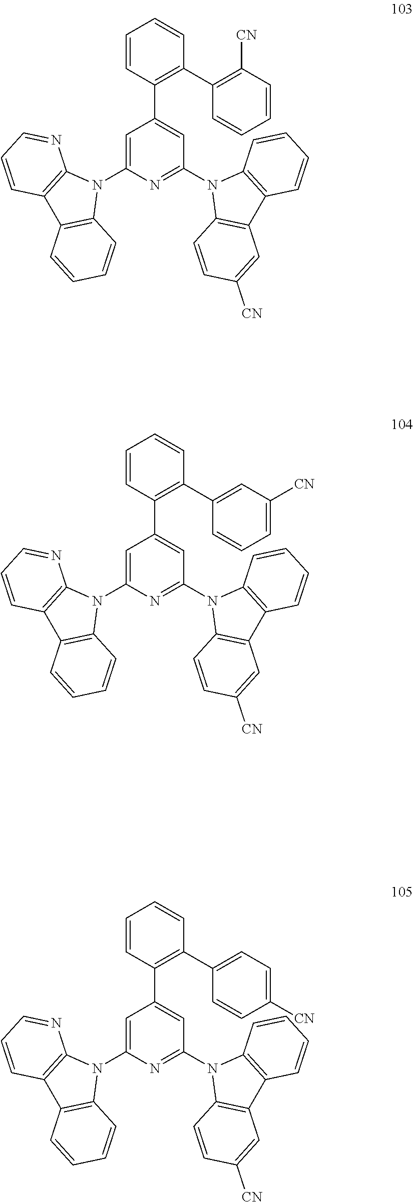 [SMILES: N#Cc1ccc2c(c1)c1ccccc1n2-c1cc(-c2ccccc2-c2ccccc2C#N)cc(-n2c3ccccc3c3cccnc32)n1 ; N#Cc1cccc(-c2ccccc2-c2cc(-n3c4ccccc4c4cc(C#N)ccc43)nc(-n3c4ccccc4c4cccnc43)c2)c1 ; N#Cc1ccc(-c2ccccc2-c2cc(-n3c4ccccc4c4cc(C#N)ccc43)nc(-n3c4ccccc4c4cccnc43)c2)cc1]

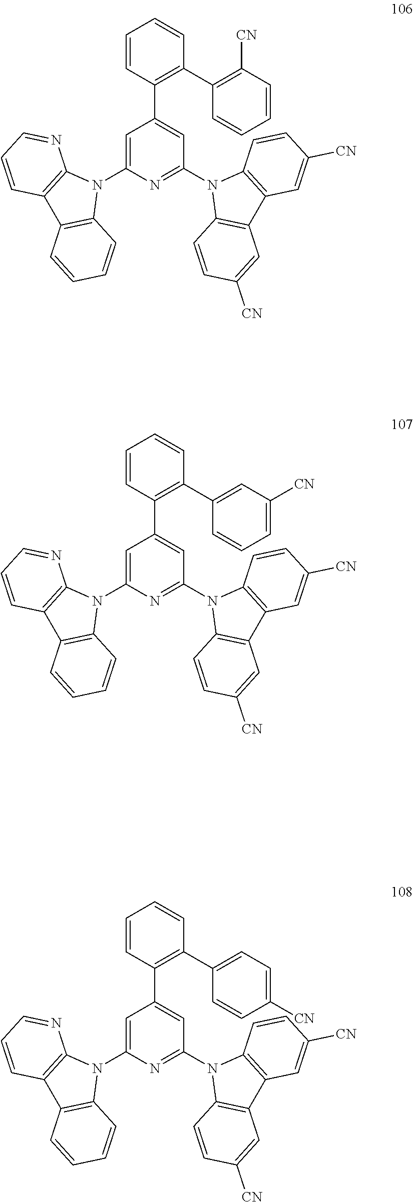 [SMILES: N#Cc1ccc2c(c1)c1cc(C#N)ccc1n2-c1cc(-c2ccccc2-c2ccccc2C#N)cc(-n2c3ccccc3c3cccnc32)n1 ; N#Cc1cccc(-c2ccccc2-c2cc(-n3c4ccc(C#N)cc4c4cc(C#N)ccc43)nc(-n3c4ccccc4c4cccnc43)c2)c1 ; N#Cc1ccc(-c2ccccc2-c2cc(-n3c4ccc(C#N)cc4c4cc(C#N)ccc43)nc(-n3c4ccccc4c4cccnc43)c2)cc1]

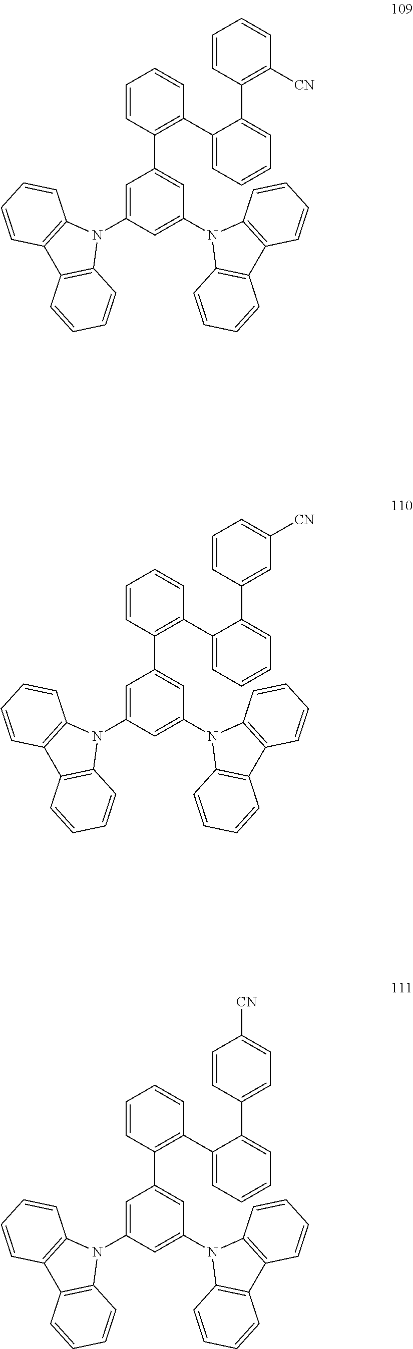 [SMILES: N#Cc1ccccc1-c1ccccc1-c1ccccc1-c1cc(-n2c3ccccc3c3ccccc32)cc(-n2c3ccccc3c3ccccc32)c1 ; N#Cc1cccc(-c2ccccc2-c2ccccc2-c2cc(-n3c4ccccc4c4ccccc43)cc(-n3c4ccccc4c4ccccc43)c2)c1 ; N#Cc1ccc(-c2ccccc2-c2ccccc2-c2cc(-n3c4ccccc4c4ccccc43)cc(-n3c4ccccc4c4ccccc43)c2)cc1]

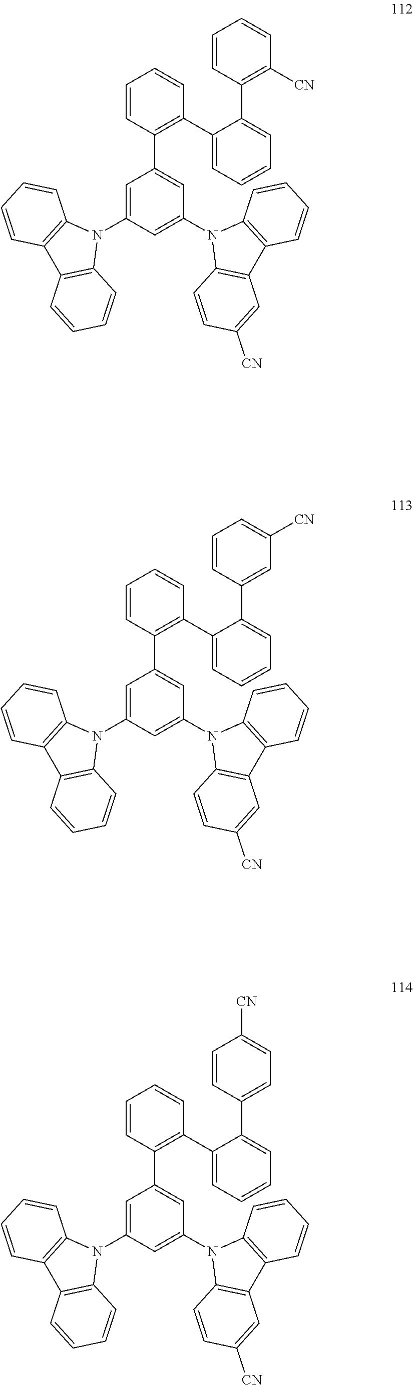 [SMILES: N#Cc1ccc2c(c1)c1ccccc1n2-c1cc(-c2ccccc2-c2ccccc2-c2ccccc2C#N)cc(-n2c3ccccc3c3ccccc32)c1 ; N#Cc1cccc(-c2ccccc2-c2ccccc2-c2cc(-n3c4ccccc4c4ccccc43)cc(-n3c4ccccc4c4cc(C#N)ccc43)c2)c1 ; N#Cc1ccc(-c2ccccc2-c2ccccc2-c2cc(-n3c4ccccc4c4ccccc43)cc(-n3c4ccccc4c4cc(C#N)ccc43)c2)cc1]

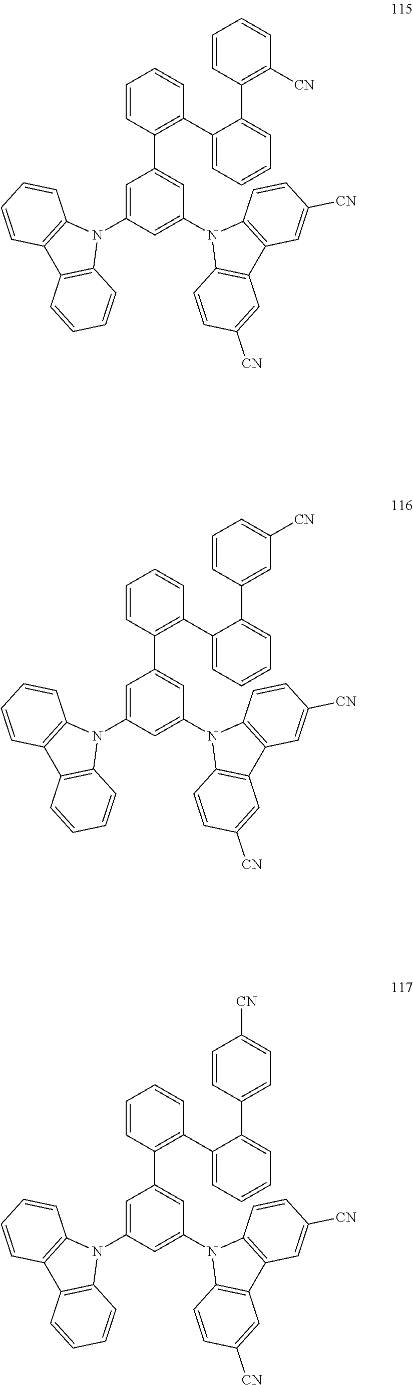 [SMILES: N#Cc1ccc2c(c1)c1cc(C#N)ccc1n2-c1cc(-c2ccccc2-c2ccccc2-c2ccccc2C#N)cc(-n2c3ccccc3c3ccccc32)c1 ; N#Cc1cccc(-c2ccccc2-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2ccccc2-c2cc(-n3c4ccccc4c4ccccc43)cc(-n3c4ccc(C#N)cc4c4cc(C#N)ccc43)c2)cc1]

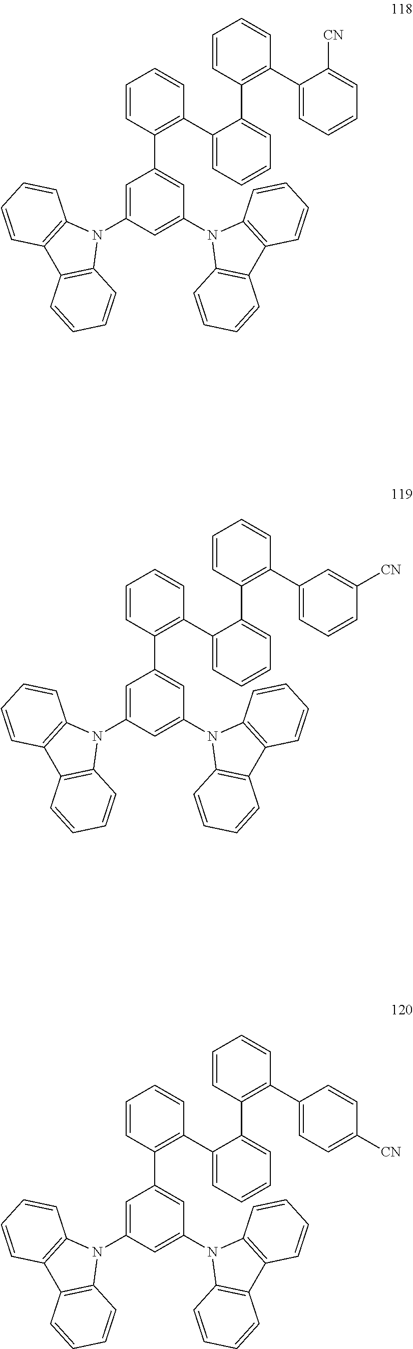 [SMILES: N#Cc1ccccc1-c1ccccc1-c1ccccc1-c1ccccc1-c1cc(-n2c3ccccc3c3ccccc32)cc(-n2c3ccccc3c3ccccc32)c1 ; N#Cc1cccc(-c2ccccc2-c2ccccc2-c2ccccc2-c2cc(-n3c4ccccc4c4ccccc43)cc(-n3c4ccccc4c4ccccc43)c2)c1 ; N#Cc1ccc(-c2ccccc2-c2ccccc2-c2ccccc2-c2cc(-n3c4ccccc4c4ccccc43)cc(-n3c4ccccc4c4ccccc43)c2)cc1]

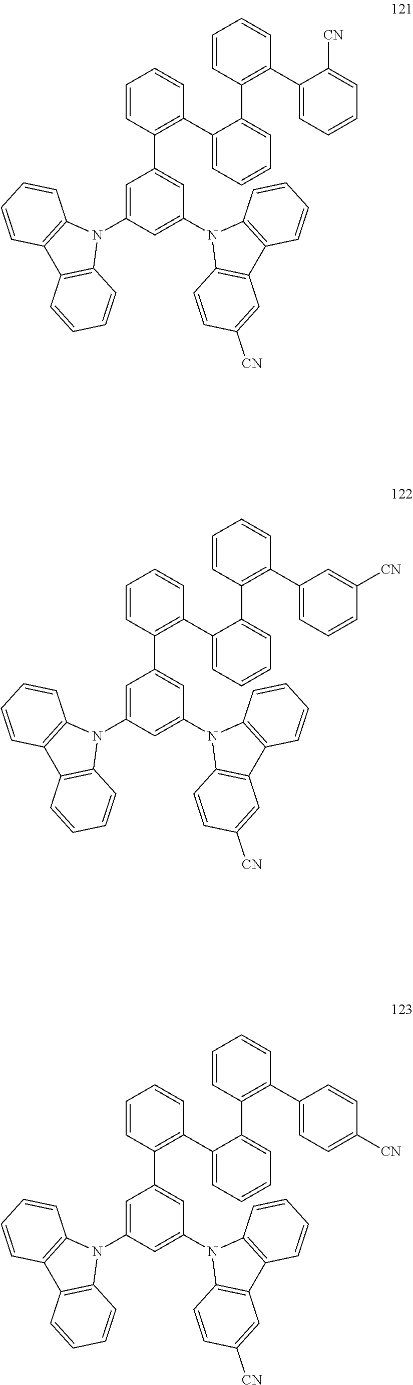 [SMILES: N#Cc1ccc2c(c1)c1ccccc1n2-c1cc(-c2ccccc2-c2ccccc2-c2ccccc2-c2ccccc2C#N)cc(-n2c3ccccc3c3ccccc32)c1 ; N#Cc1cccc(-c2ccccc2-c2ccccc2-c2ccccc2-c2cc(-n3c4ccccc4c4ccccc43)cc(-n3c4ccccc4c4cc(C#N)ccc43)c2)c1 ; N#Cc1ccc(-c2ccccc2-c2ccccc2-c2ccccc2-c2cc(-n3c4ccccc4c4ccccc43)cc(-n3c4ccccc4c4cc(C#N)ccc43)c2)cc1]

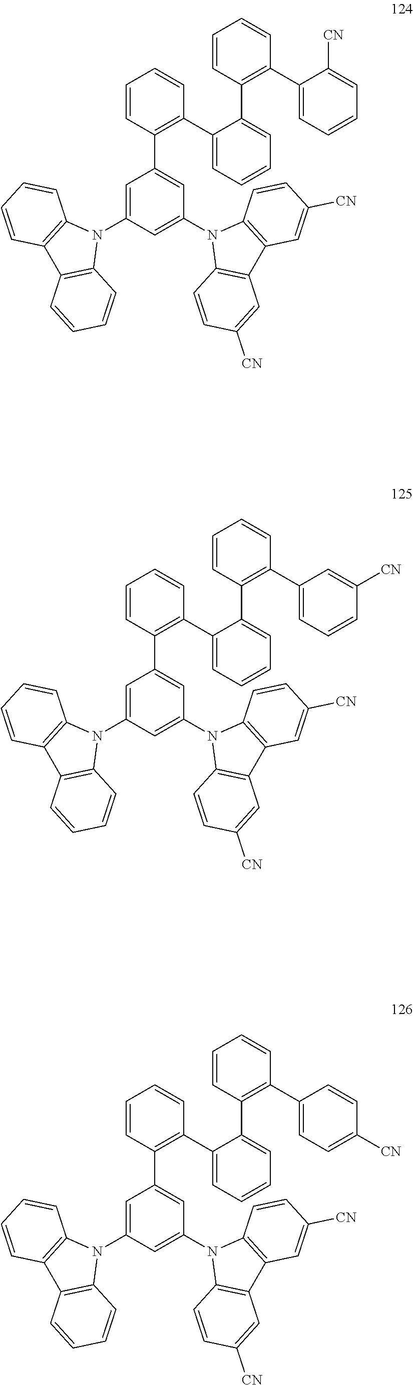 [SMILES: N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)c1 ; N#Cc1cccc(-c2ccccc2-c2ccccc2-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2ccccc2-c2ccccc2-c2cc(-n3c4ccccc4c4ccccc43)cc(-n3c4ccc(C#N)cc4c4cc(C#N)ccc43)c2)cc1]

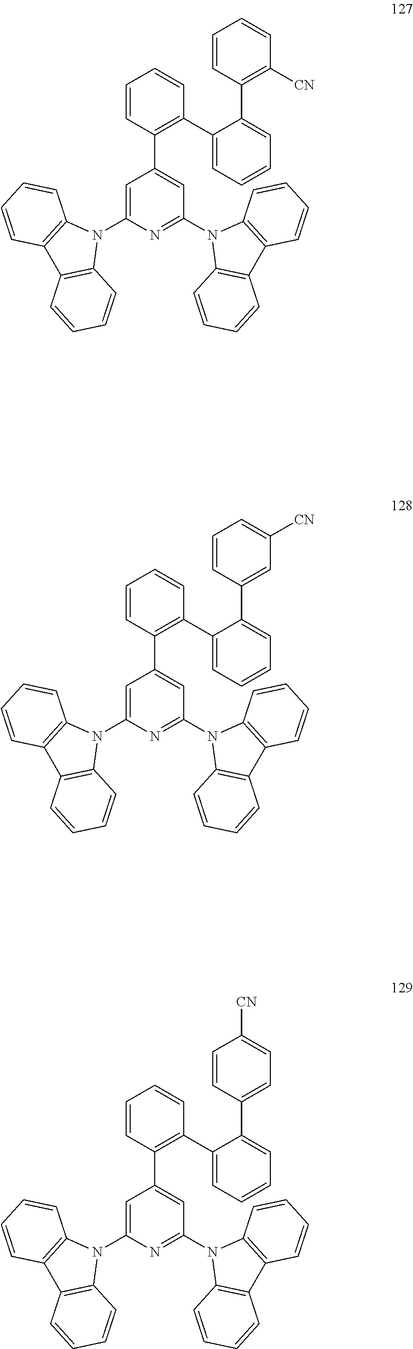 [SMILES: N#Cc1ccccc1-c1ccccc1-c1ccccc1-c1cc(-n2c3ccccc3c3ccccc32)nc(-n2c3ccccc3c3ccccc32)c1 ; N#Cc1cccc(-c2ccccc2-c2ccccc2-c2cc(-n3c4ccccc4c4ccccc43)nc(-n3c4ccccc4c4ccccc43)c2)c1 ; N#Cc1ccc(-c2ccccc2-c2ccccc2-c2cc(-n3c4ccccc4c4ccccc43)nc(-n3c4ccccc4c4ccccc43)c2)cc1]

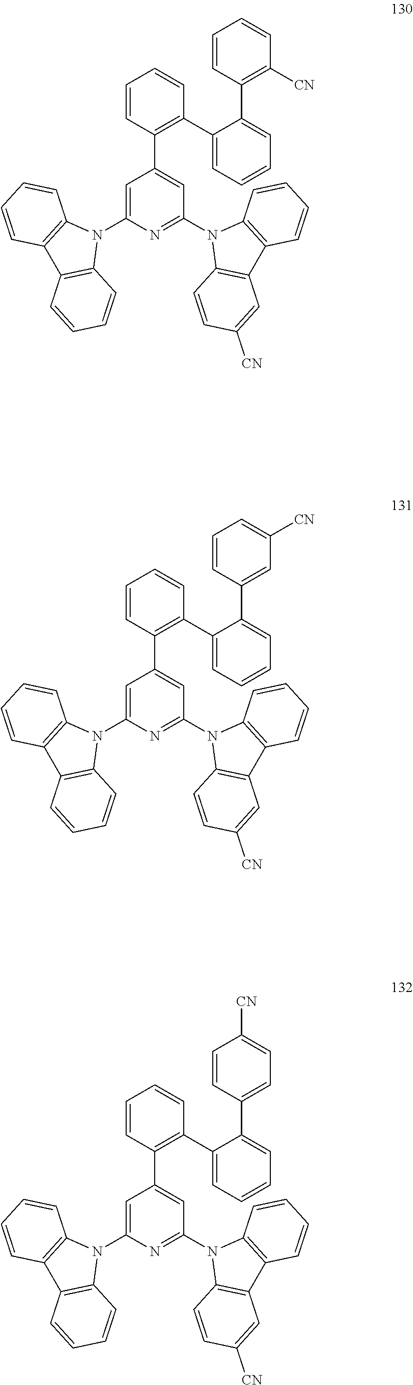 [SMILES: N#Cc1ccc2c(c1)c1ccccc1n2-c1cc(-c2ccccc2-c2ccccc2-c2ccccc2C#N)cc(-n2c3ccccc3c3ccccc32)n1 ; N#Cc1cccc(-c2ccccc2-c2ccccc2-c2cc(-n3c4ccccc4c4ccccc43)nc(-n3c4ccccc4c4cc(C#N)ccc43)c2)c1 ; N#Cc1ccc(-c2ccccc2-c2ccccc2-c2cc(-n3c4ccccc4c4ccccc43)nc(-n3c4ccccc4c4cc(C#N)ccc43)c2)cc1]

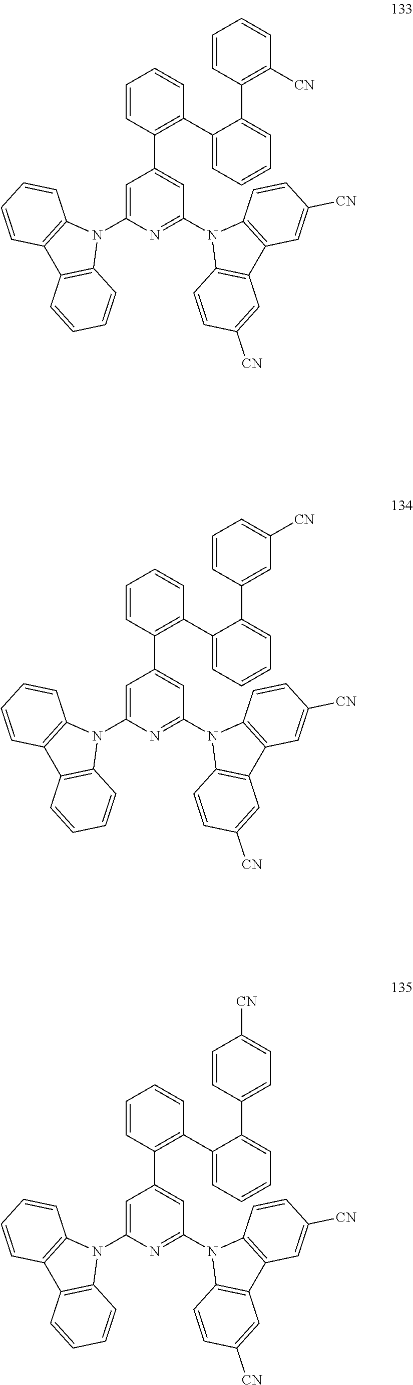 [SMILES: N#Cc1ccc2c(c1)c1cc(C#N)ccc1n2-c1cc(-c2ccccc2-c2ccccc2-c2ccccc2C#N)cc(-n2c3ccccc3c3ccccc32)n1 ; N#Cc1cccc(-c2ccccc2-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2ccccc2-c2cc(-n3c4ccccc4c4ccccc43)nc(-n3c4ccc(C#N)cc4c4cc(C#N)ccc43)c2)cc1]

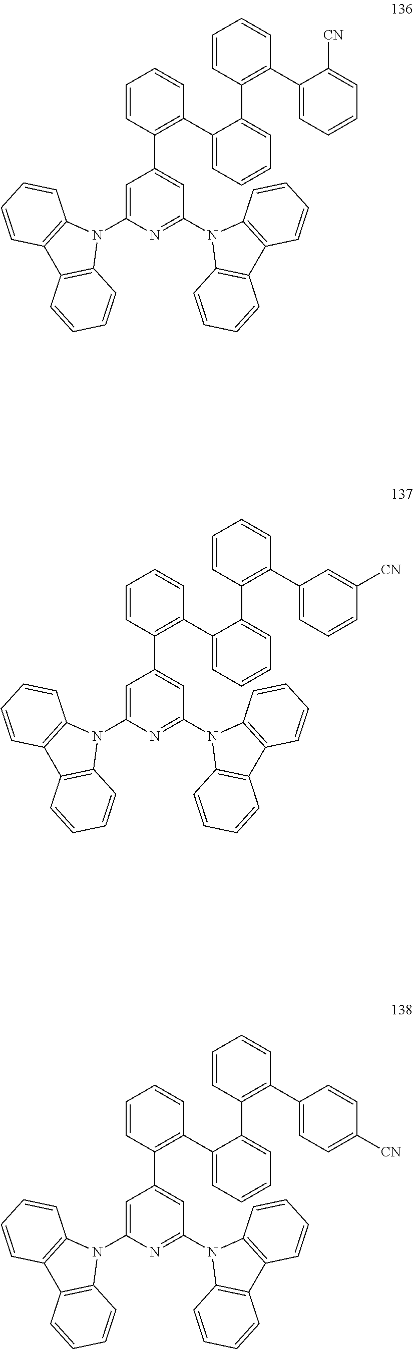 [SMILES: N#Cc1ccccc1-c1ccccc1-c1ccccc1-c1ccccc1-c1cc(-n2c3ccccc3c3ccccc32)nc(-n2c3ccccc3c3ccccc32)c1 ; N#Cc1cccc(-c2ccccc2-c2ccccc2-c2ccccc2-c2cc(-n3c4ccccc4c4ccccc43)nc(-n3c4ccccc4c4ccccc43)c2)c1 ; N#Cc1ccc(-c2ccccc2-c2ccccc2-c2ccccc2-c2cc(-n3c4ccccc4c4ccccc43)nc(-n3c4ccccc4c4ccccc43)c2)cc1]

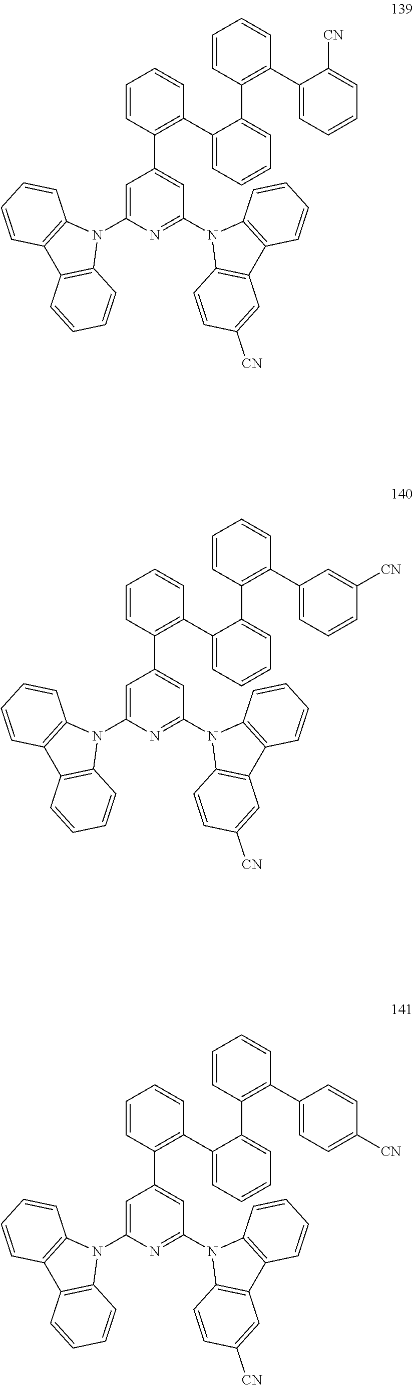 [SMILES: N#Cc1ccc2c(c1)c1ccccc1n2-c1cc(-c2ccccc2-c2ccccc2-c2ccccc2-c2ccccc2C#N)cc(-n2c3ccccc3c3ccccc32)n1 ; N#Cc1cccc(-c2ccccc2-c2ccccc2-c2ccccc2-c2cc(-n3c4ccccc4c4ccccc43)nc(-n3c4ccccc4c4cc(C#N)ccc43)c2)c1 ; N#Cc1ccc(-c2ccccc2-c2ccccc2-c2ccccc2-c2cc(-n3c4ccccc4c4ccccc43)nc(-n3c4ccccc4c4cc(C#N)ccc43)c2)cc1]

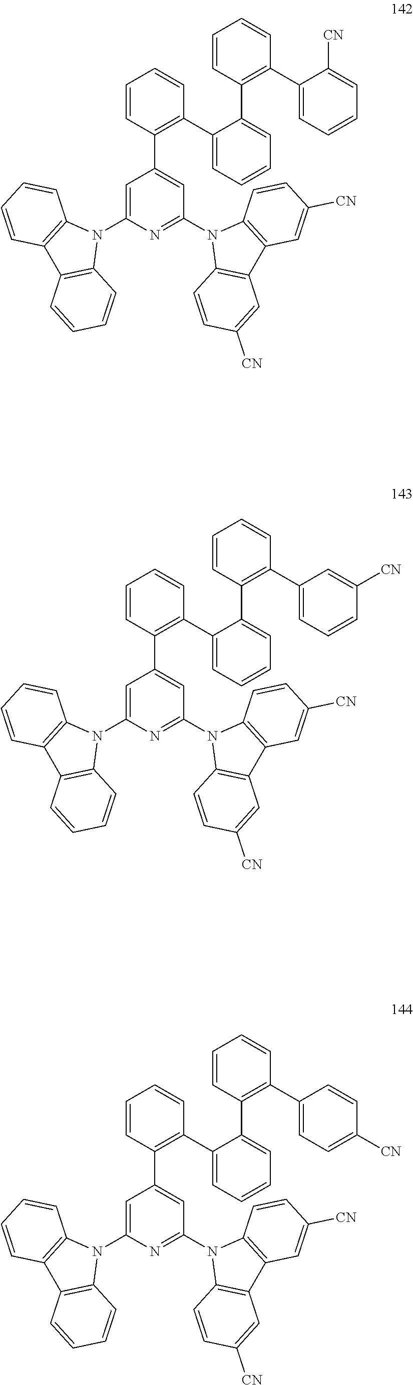 [SMILES: N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)n1 ; N#Cc1cccc(-c2ccccc2-c2ccccc2-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2ccccc2-c2ccccc2-c2cc(-n3c4ccccc4c4ccccc43)nc(-n3c4ccc(C#N)cc4c4cc(C#N)ccc43)c2)cc1]

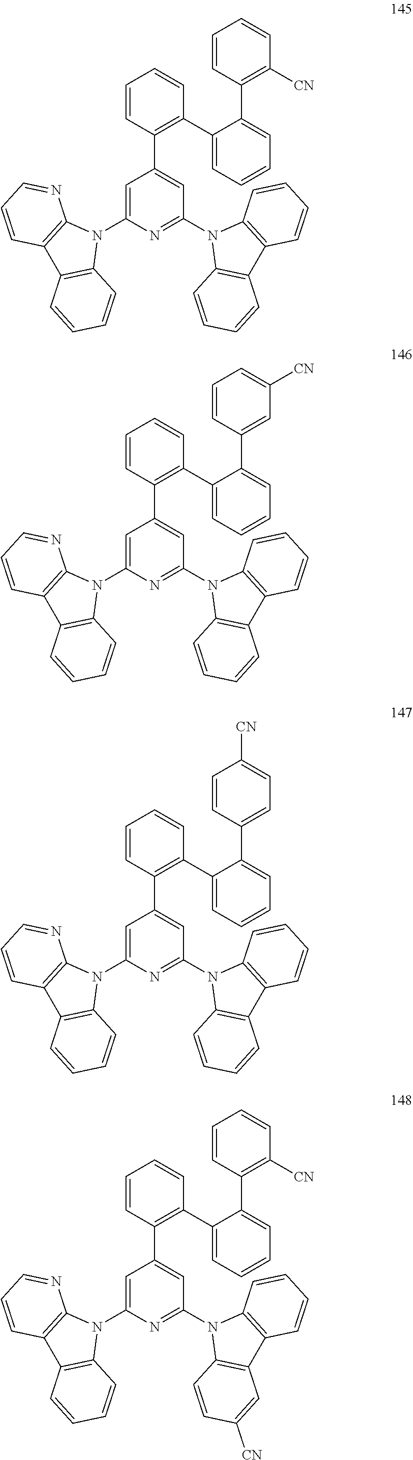

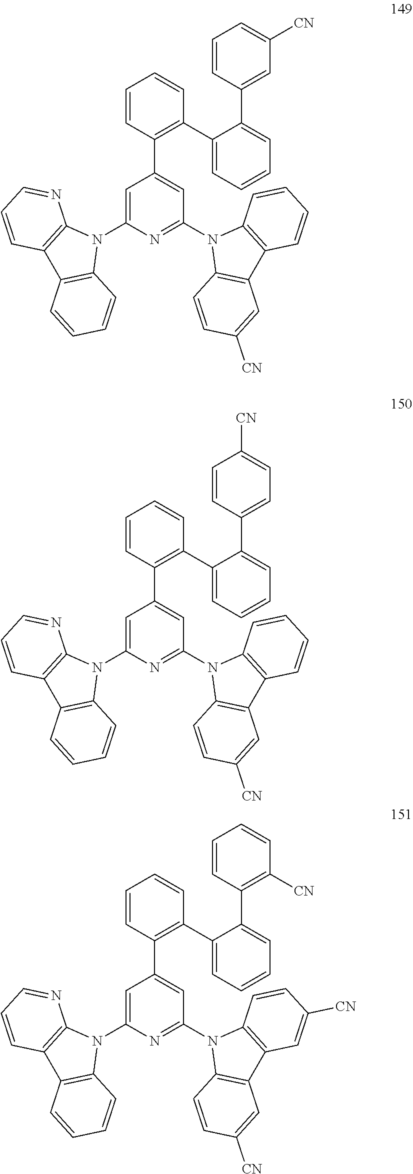

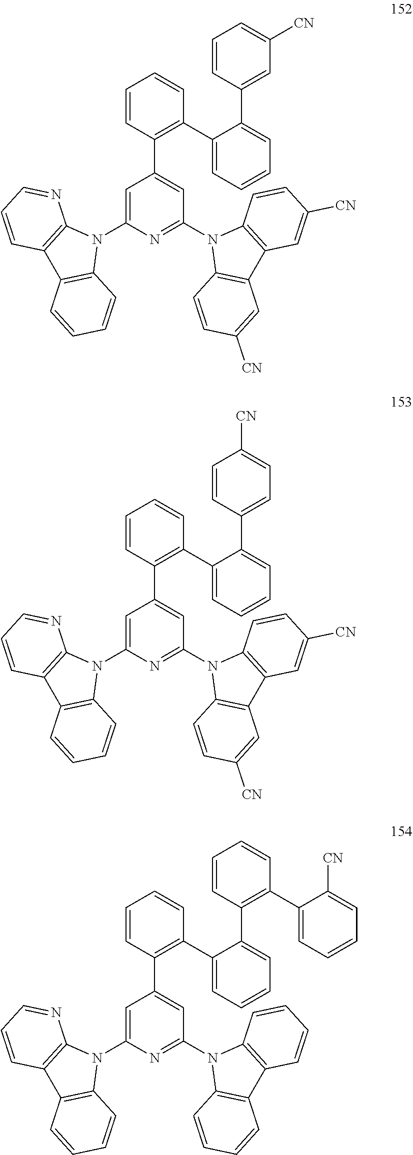 [SMILES: N#Cc1cccc(-c2ccccc2-c2ccccc2-c2cc(-n3c4ccc(C#N)cc4c4cc(C#N)ccc43)nc(-n3c4ccccc4c4cccnc43)c2)c1 ; N#Cc1ccc(-c2ccccc2-c2ccccc2-c2cc(-n3c4ccc(C#N)cc4c4cc(C#N)ccc43)nc(-n3c4ccccc4c4cccnc43)c2)cc1 ; N#Cc1ccccc1-c1ccccc1-c1ccccc1-c1ccccc1-c1cc(-n2c3ccccc3c3ccccc32)nc(-n2c3ccccc3c3cccnc32)c1]

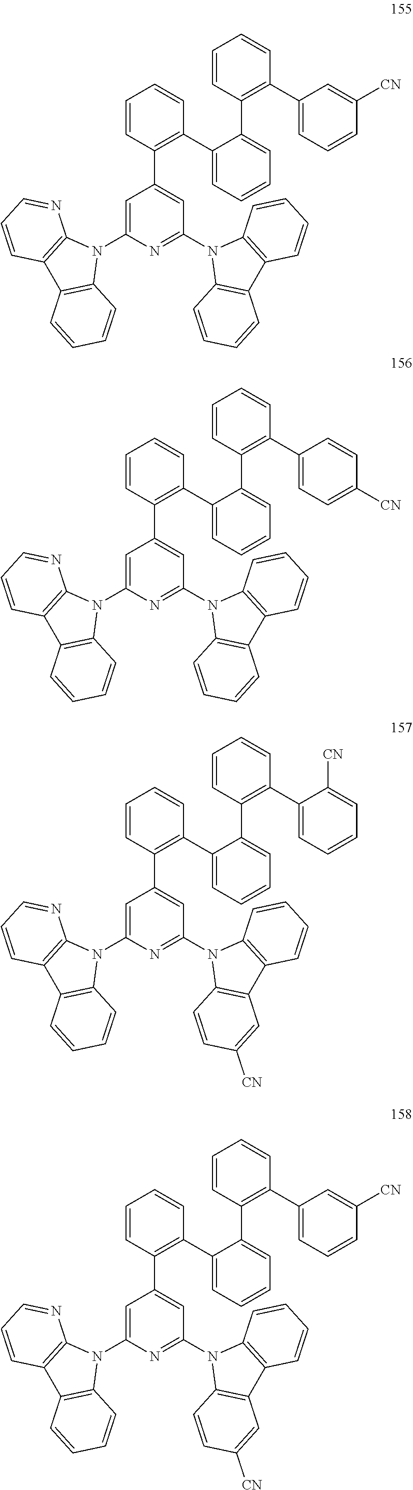

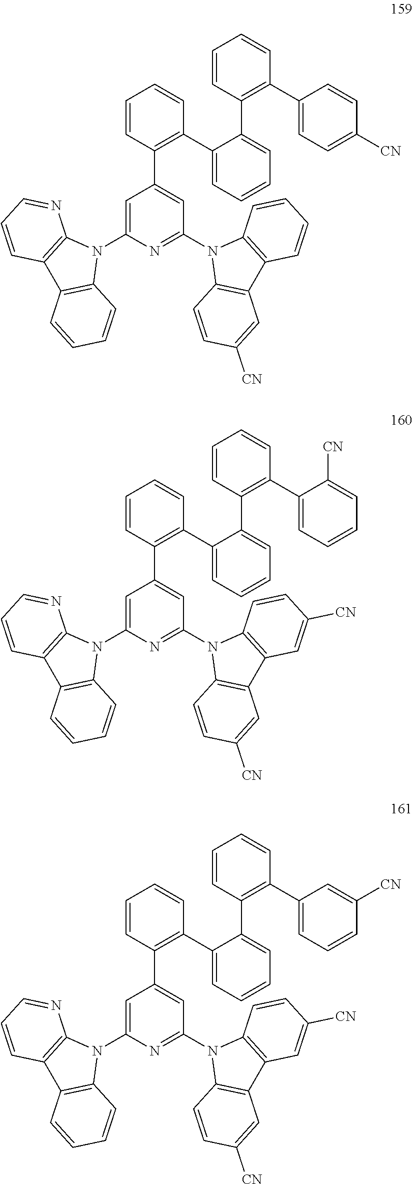 [SMILES: N#Cc1ccc(-c2ccccc2-c2ccccc2-c2ccccc2-c2cc(-n3c4ccccc4c4cc(C#N)ccc43)nc(-n3c4ccccc4c4cccnc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3cccnc32)n1 ; N#Cc1cccc(-c2ccccc2-c2ccccc2-c2ccccc2-c2cc(-n3c4ccc(C#N)cc4c4cc(C#N)ccc43)nc(-n3c4ccccc4c4cccnc43)c2)c1]

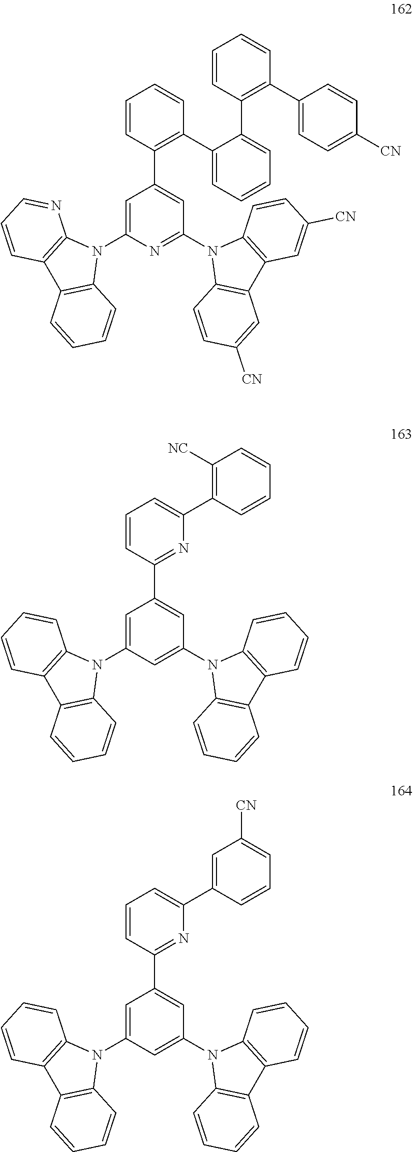

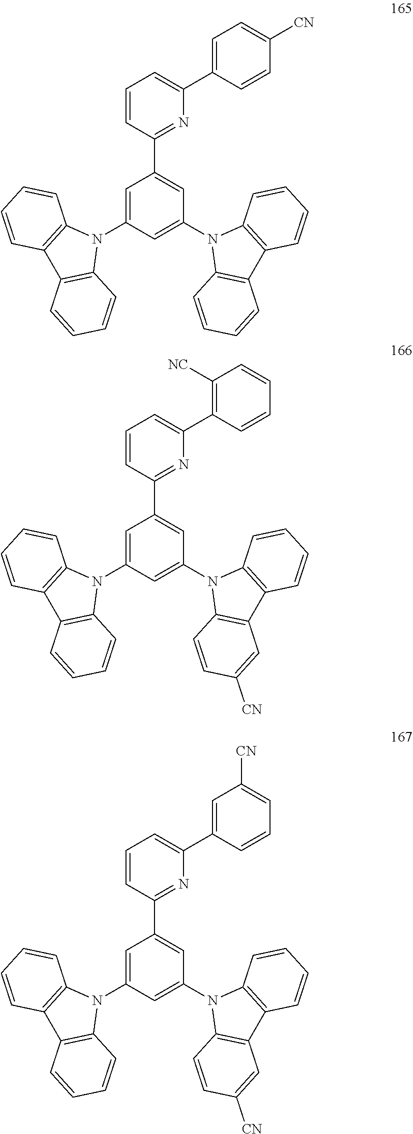

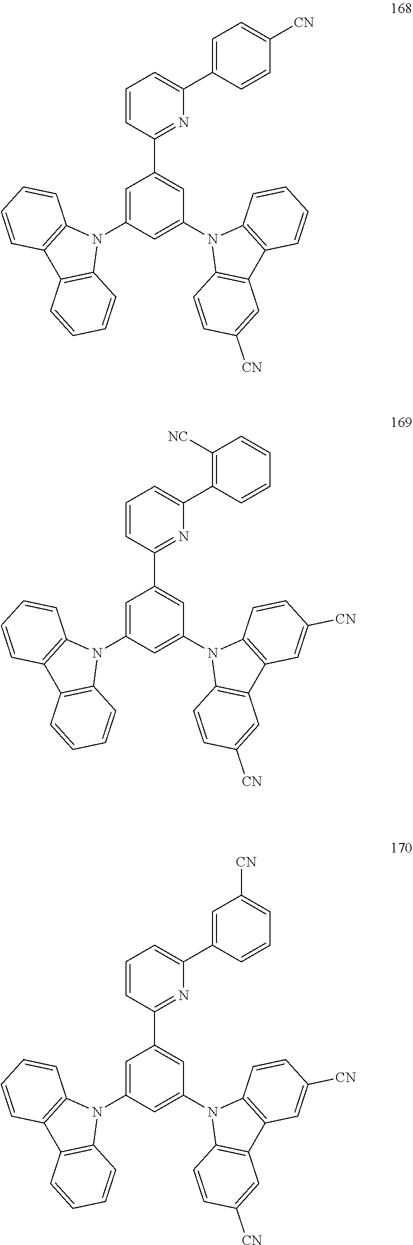 [SMILES: N#Cc1ccc(-c2cccc(-c3cc(-n4c5ccccc5c5ccccc54)cc(-n4c5ccccc5c5cc(C#N)ccc54)c3)n2)cc1 ; N#Cc1ccc2c(c1)c1cc(C#N)ccc1n2-c1cc(-c2cccc(-c3ccccc3C#N)n2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)n2)c1]

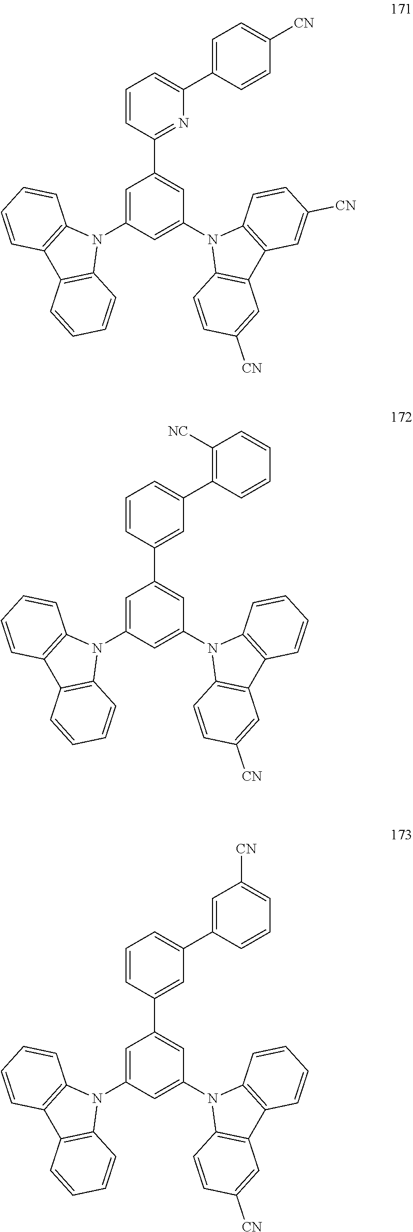 [SMILES: N#Cc1ccc(-c2cccc(-c3cc(-n4c5ccccc5c5ccccc54)cc(-n4c5ccc(C#N)cc5c5cc(C#N)ccc54)c3)n2)cc1 ; N#Cc1ccc2c(c1)c1ccccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccccc5c5cc(C#N)ccc54)c3)c2)c1]

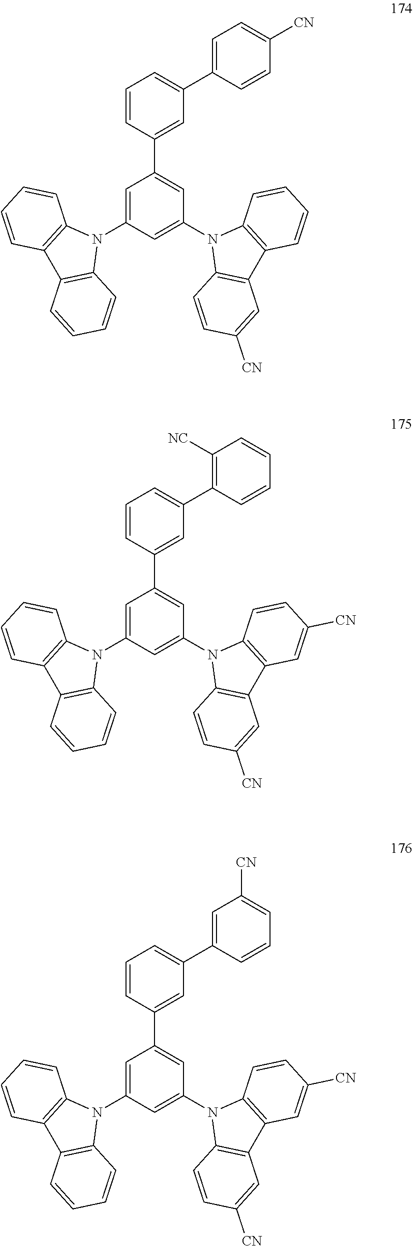 [SMILES: N#Cc1ccc(-c2cccc(-c3cc(-n4c5ccccc5c5ccccc54)cc(-n4c5ccccc5c5cc(C#N)ccc54)c3)c2)cc1 ; N#Cc1ccc2c(c1)c1cc(C#N)ccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)c2)c1]

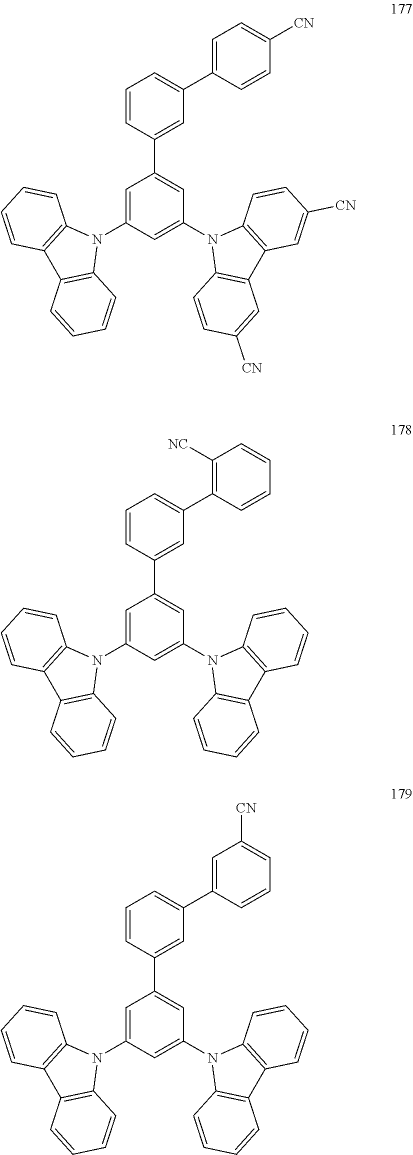 [SMILES: N#Cc1ccc(-c2cccc(-c3cc(-n4c5ccccc5c5ccccc54)cc(-n4c5ccc(C#N)cc5c5cc(C#N)ccc54)c3)c2)cc1 ; N#Cc1ccccc1-c1cccc(-c2cc(-n3c4ccccc4c4ccccc43)cc(-n3c4ccccc4c4ccccc43)c2)c1 ; N#Cc1cccc(-c2cccc(-c3cc(-n4c5ccccc5c5ccccc54)cc(-n4c5ccccc5c5ccccc54)c3)c2)c1]

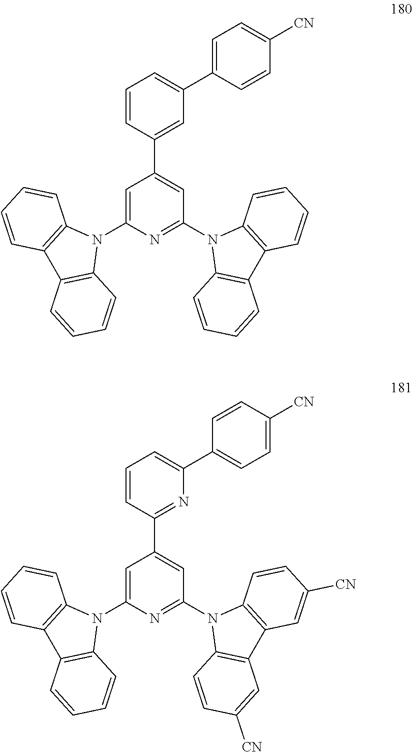 [SMILES: N#Cc1ccc(-c2cccc(-c3cc(-n4c5ccccc5c5ccccc54)nc(-n4c5ccccc5c5ccccc54)c3)c2)cc1 ; N#Cc1ccc(-c2cccc(-c3cc(-n4c5ccccc5c5ccccc54)nc(-n4c5ccc(C#N)cc5c5cc(C#N)ccc54)c3)n2)cc1]

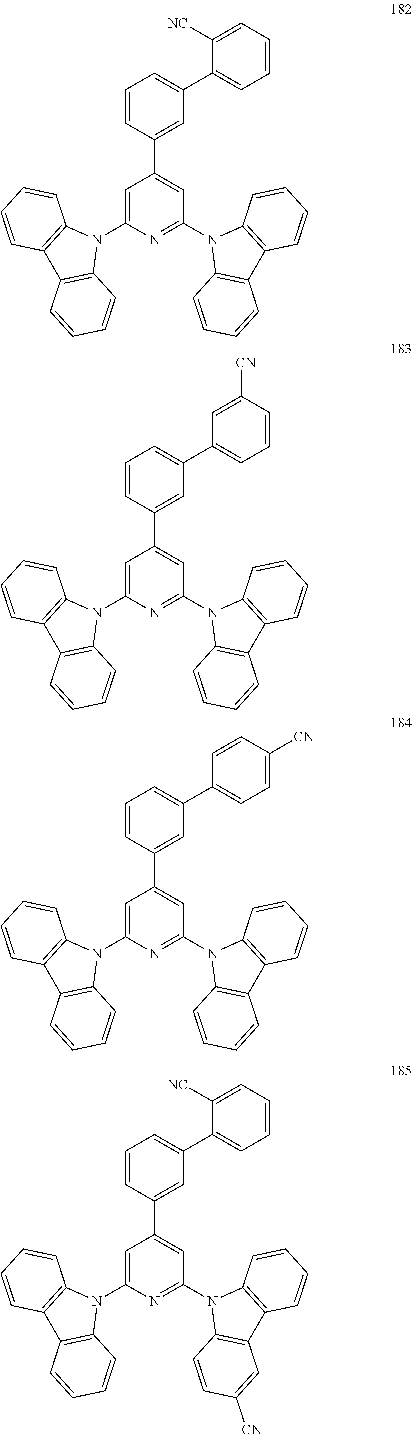

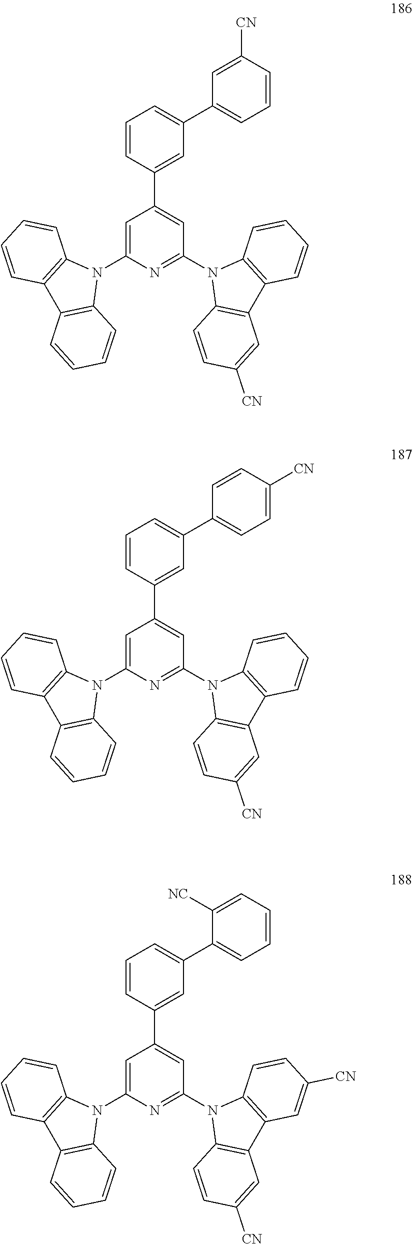 [SMILES: N#Cc1cccc(-c2cccc(-c3cc(-n4c5ccccc5c5ccccc54)nc(-n4c5ccccc5c5cc(C#N)ccc54)c3)c2)c1 ; N#Cc1ccc(-c2cccc(-c3cc(-n4c5ccccc5c5ccccc54)nc(-n4c5ccccc5c5cc(C#N)ccc54)c3)c2)cc1 ; N#Cc1ccc2c(c1)c1cc(C#N)ccc1n2-c1cc(-c2cccc(-c3ccccc3C#N)c2)cc(-n2c3ccccc3c3ccccc32)n1]

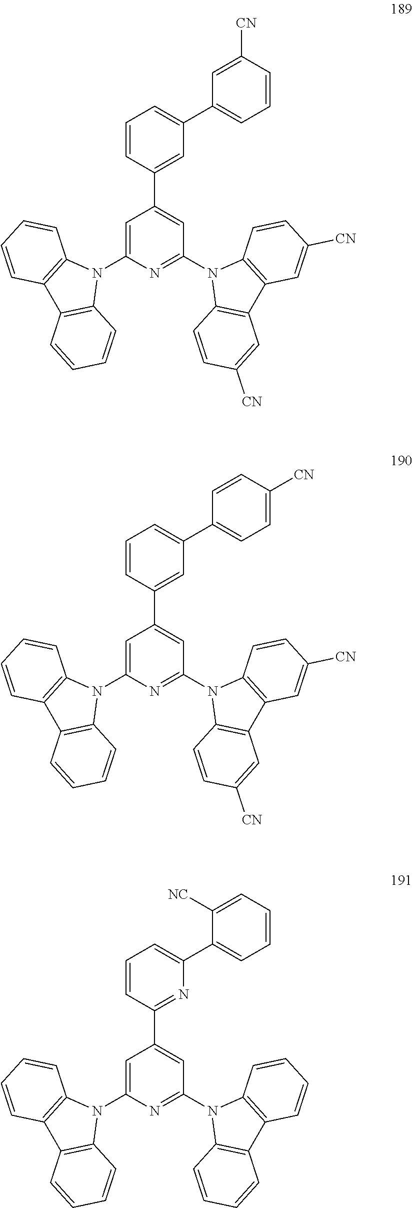 [SMILES: N#Cc1cccc(-c2cccc(-c3cc(-n4c5ccccc5c5ccccc54)nc(-n4c5ccc(C#N)cc5c5cc(C#N)ccc54)c3)c2)c1 ; N#Cc1ccc(-c2cccc(-c3cc(-n4c5ccccc5c5ccccc54)nc(-n4c5ccc(C#N)cc5c5cc(C#N)ccc54)c3)c2)cc1 ; N#Cc1ccccc1-c1cccc(-c2cc(-n3c4ccccc4c4ccccc43)nc(-n3c4ccccc4c4ccccc43)c2)n1]

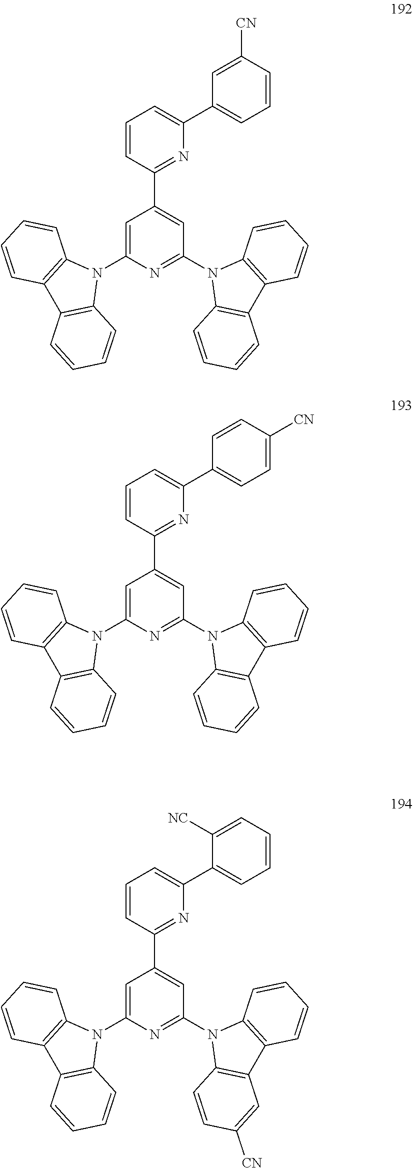 [SMILES: N#Cc1cccc(-c2cccc(-c3cc(-n4c5ccccc5c5ccccc54)nc(-n4c5ccccc5c5ccccc54)c3)n2)c1 ; N#Cc1ccc(-c2cccc(-c3cc(-n4c5ccccc5c5ccccc54)nc(-n4c5ccccc5c5ccccc54)c3)n2)cc1 ; N#Cc1ccc2c(c1)c1ccccc1n2-c1cc(-c2cccc(-c3ccccc3C#N)n2)cc(-n2c3ccccc3c3ccccc32)n1]

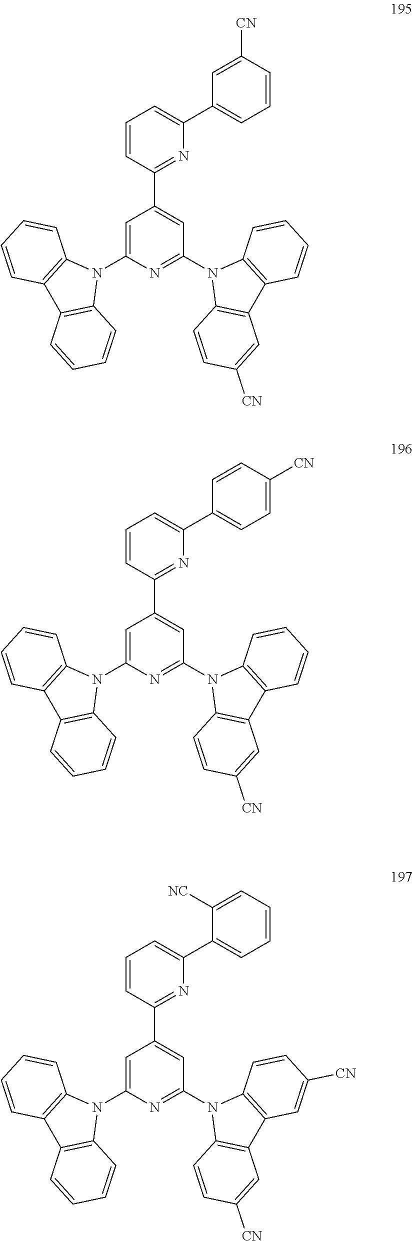 [SMILES: N#Cc1cccc(-c2cccc(-c3cc(-n4c5ccccc5c5ccccc54)nc(-n4c5ccccc5c5cc(C#N)ccc54)c3)n2)c1 ; N#Cc1ccc(-c2cccc(-c3cc(-n4c5ccccc5c5ccccc54)nc(-n4c5ccccc5c5cc(C#N)ccc54)c3)n2)cc1 ; N#Cc1ccc2c(c1)c1cc(C#N)ccc1n2-c1cc(-c2cccc(-c3ccccc3C#N)n2)cc(-n2c3ccccc3c3ccccc32)n1]

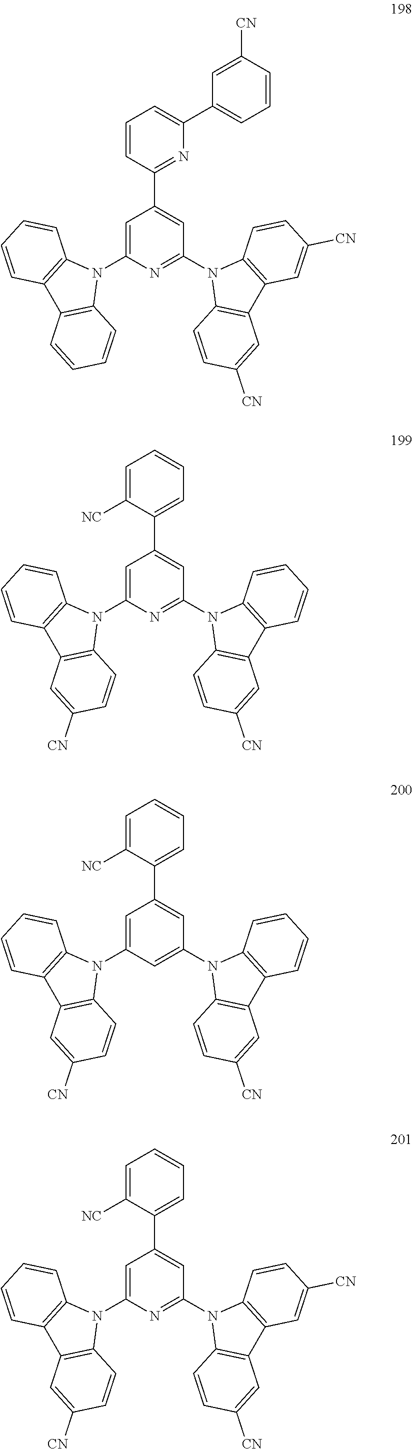

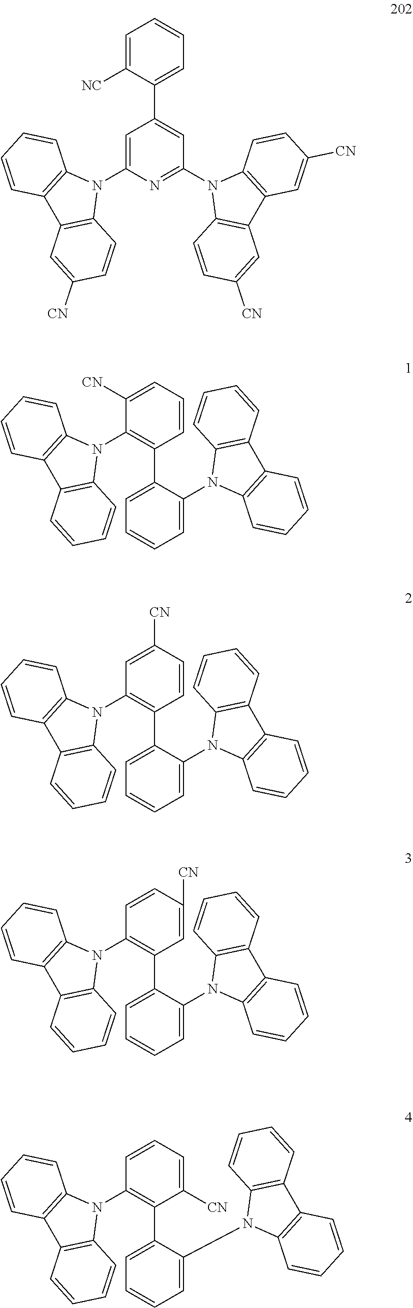

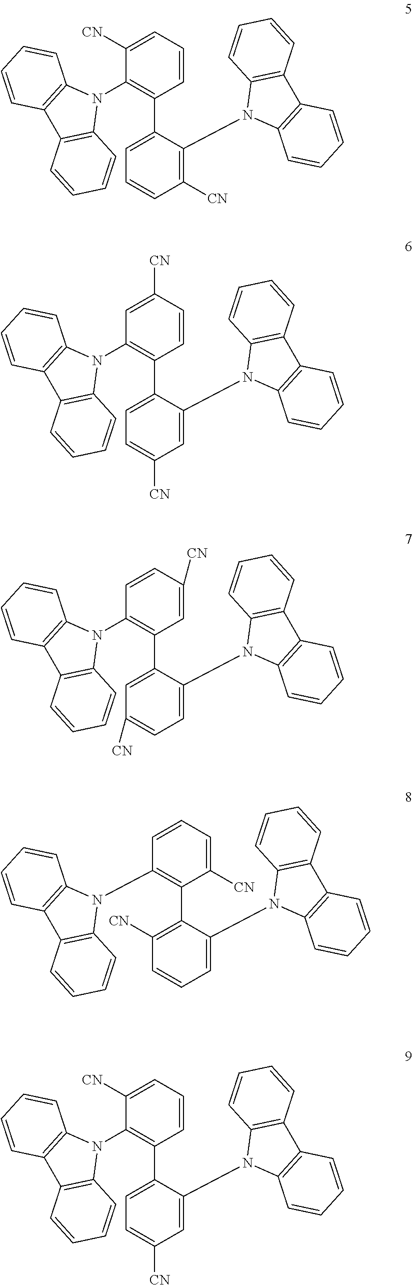

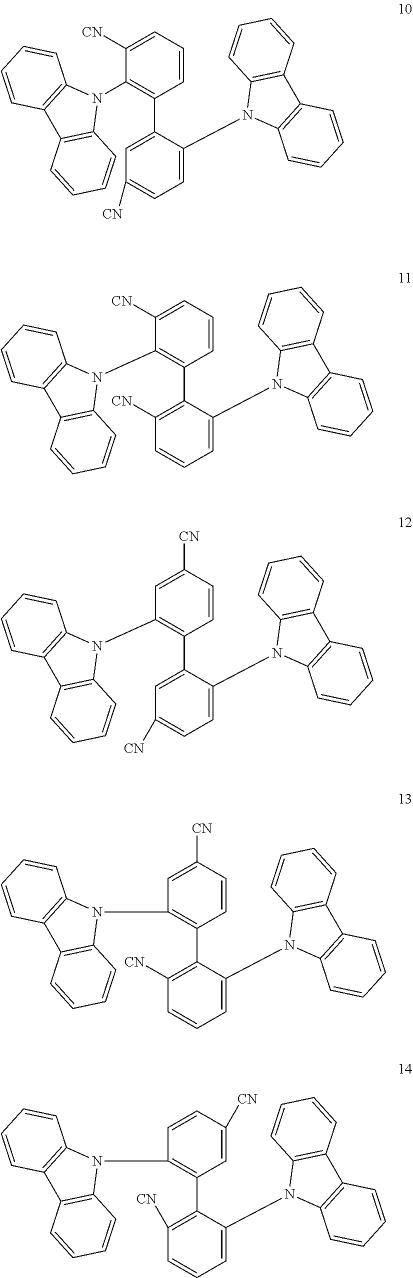

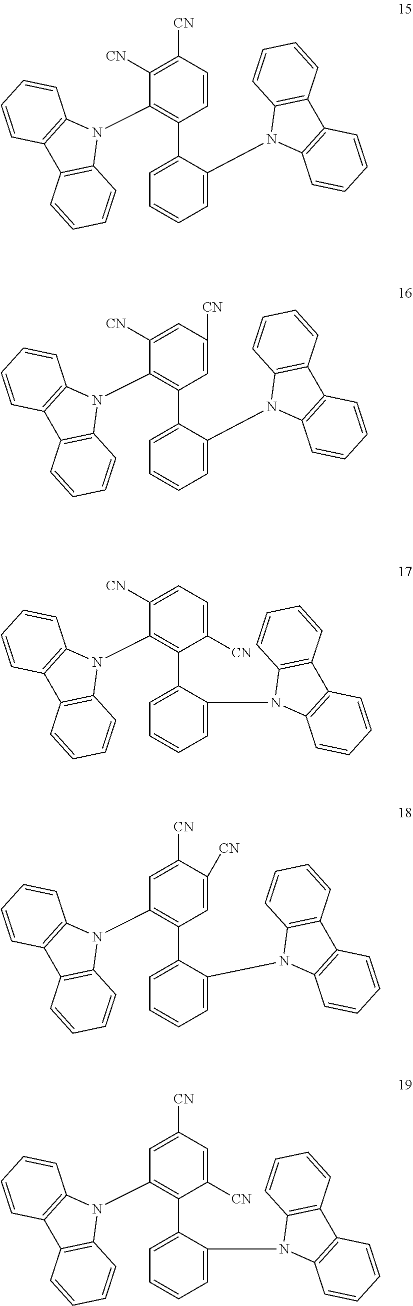

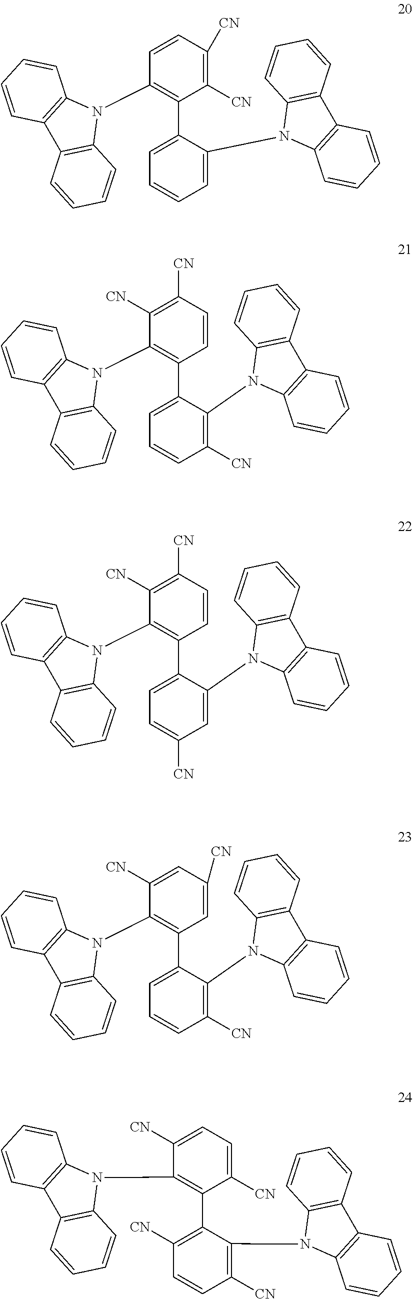

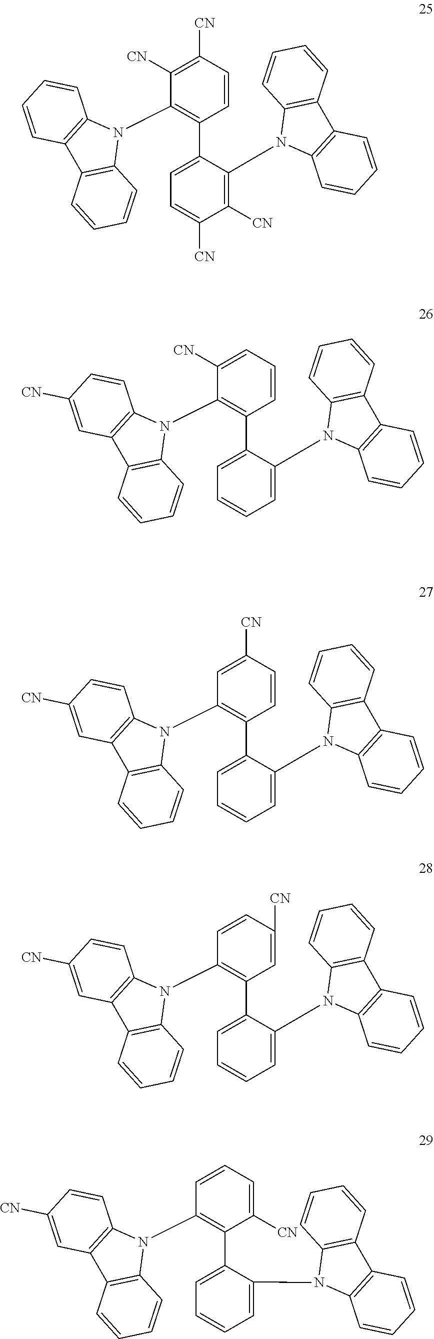

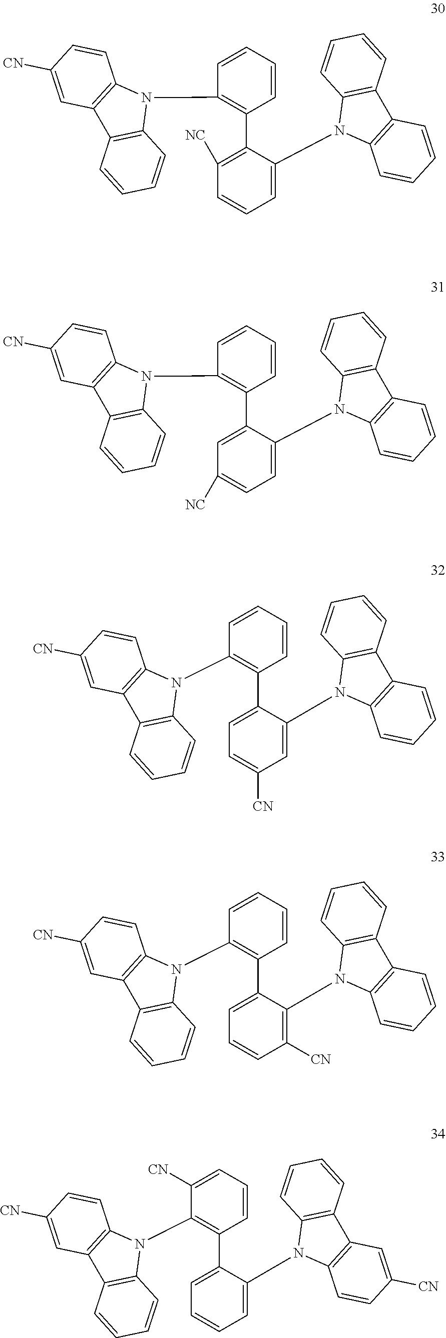

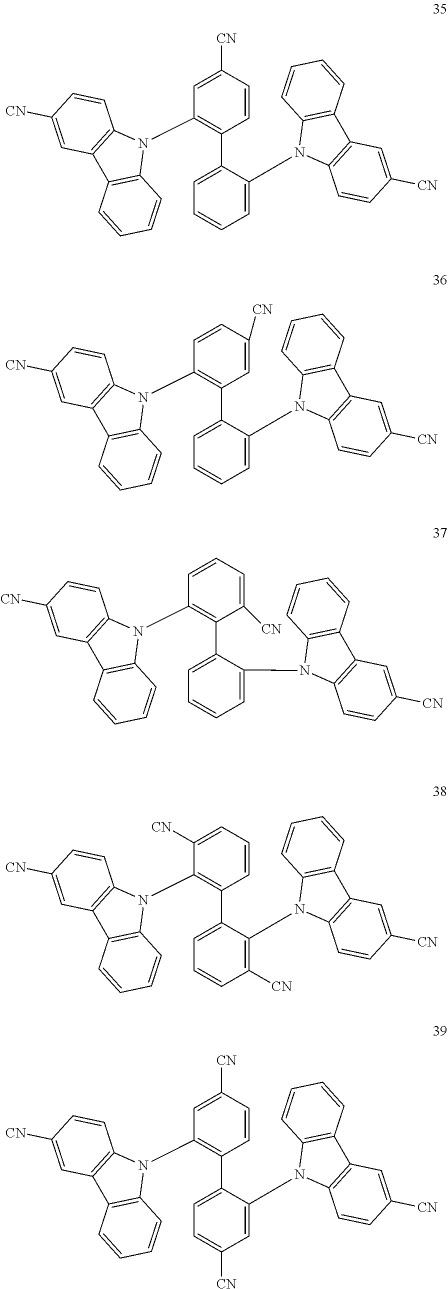

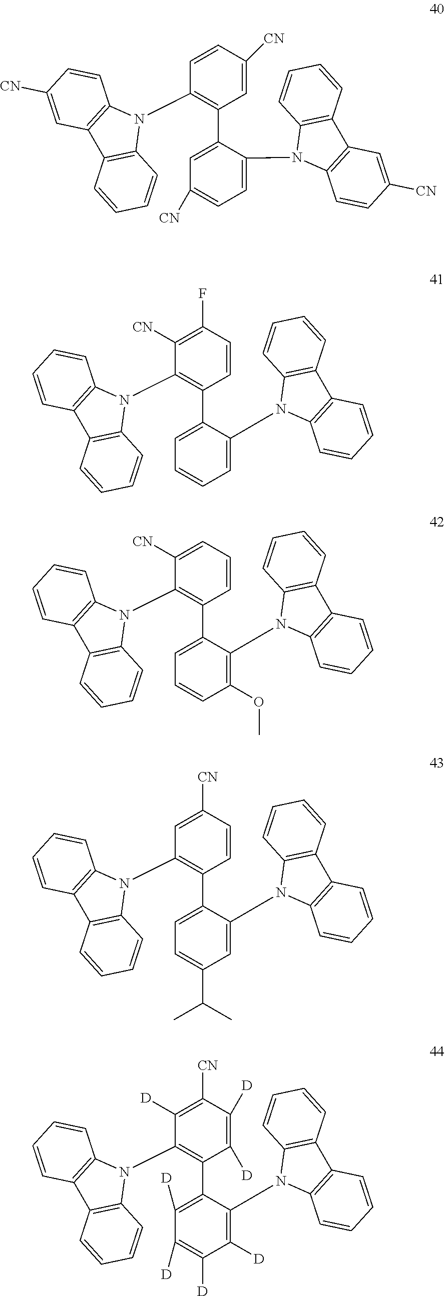

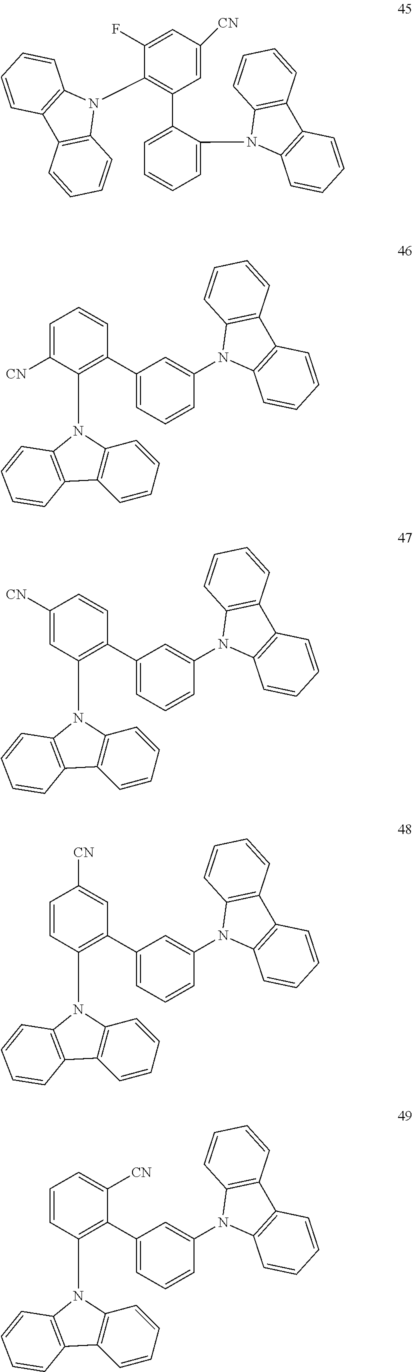

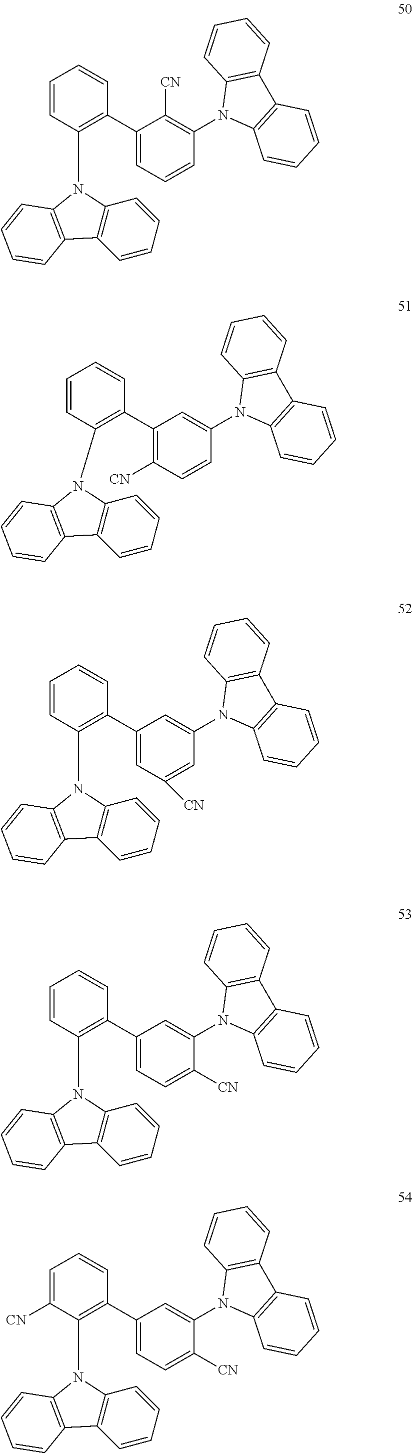

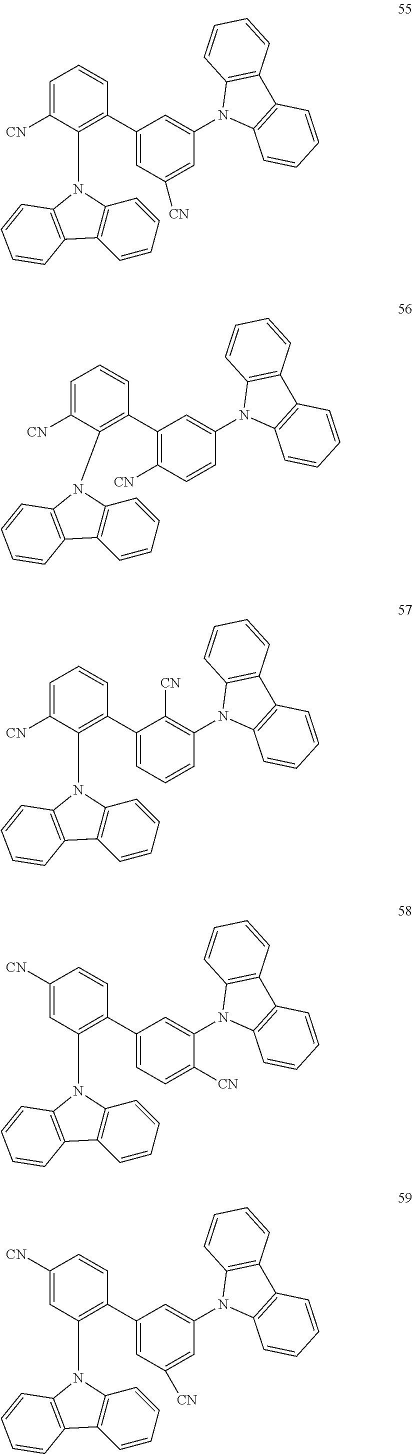

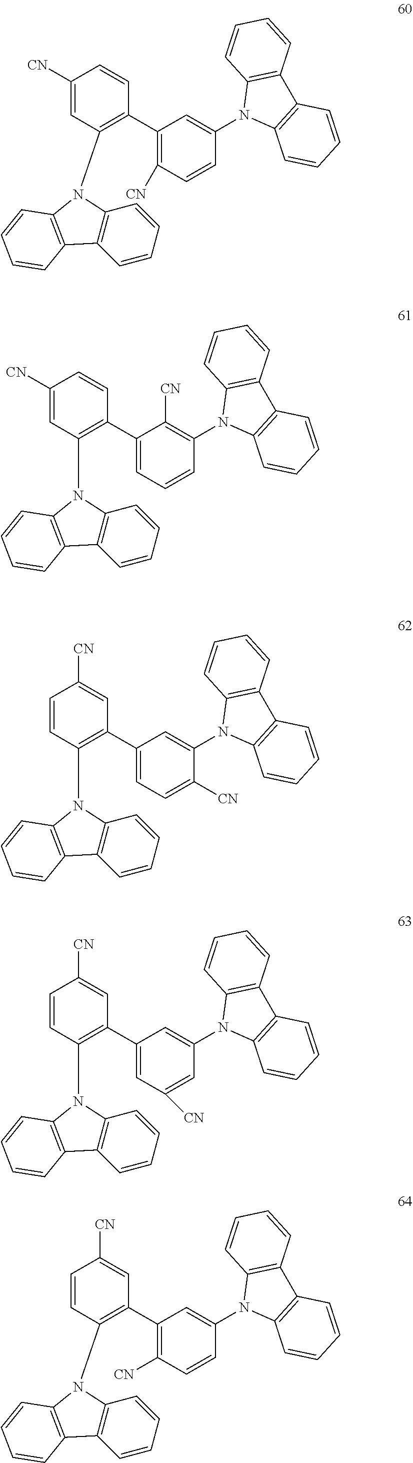

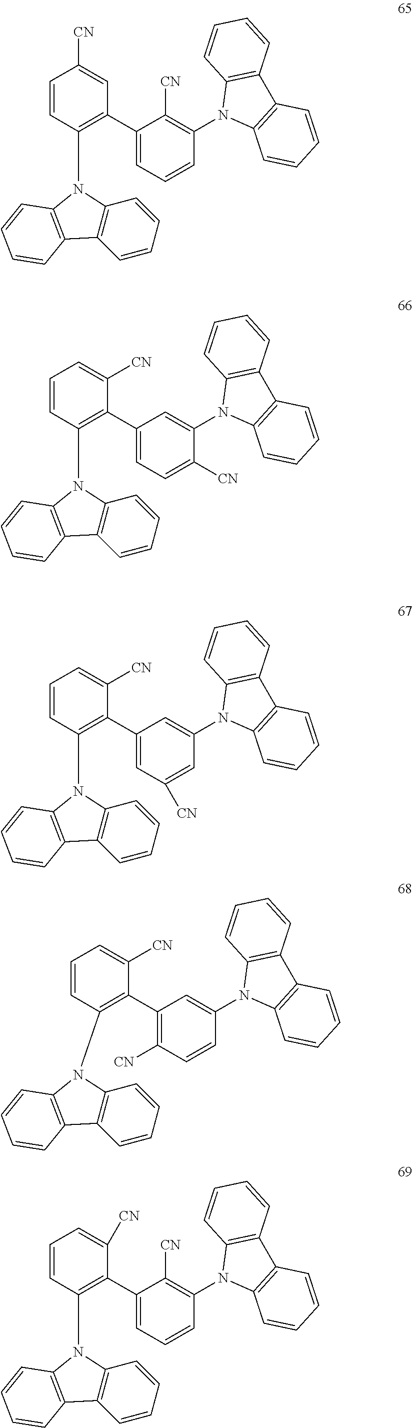

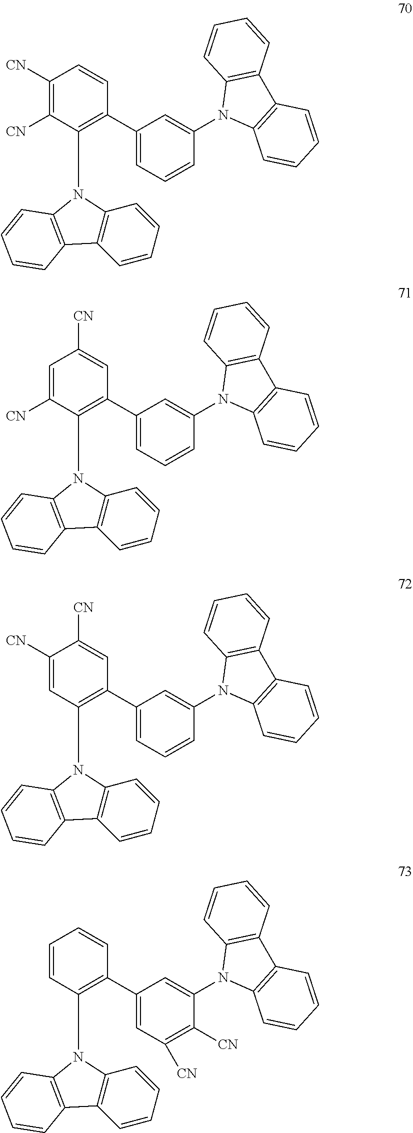

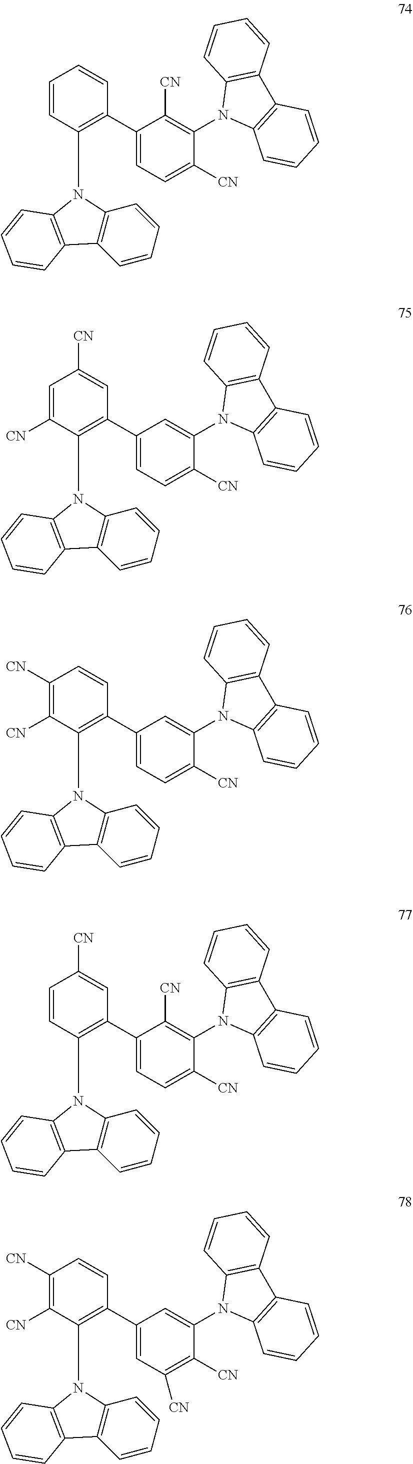

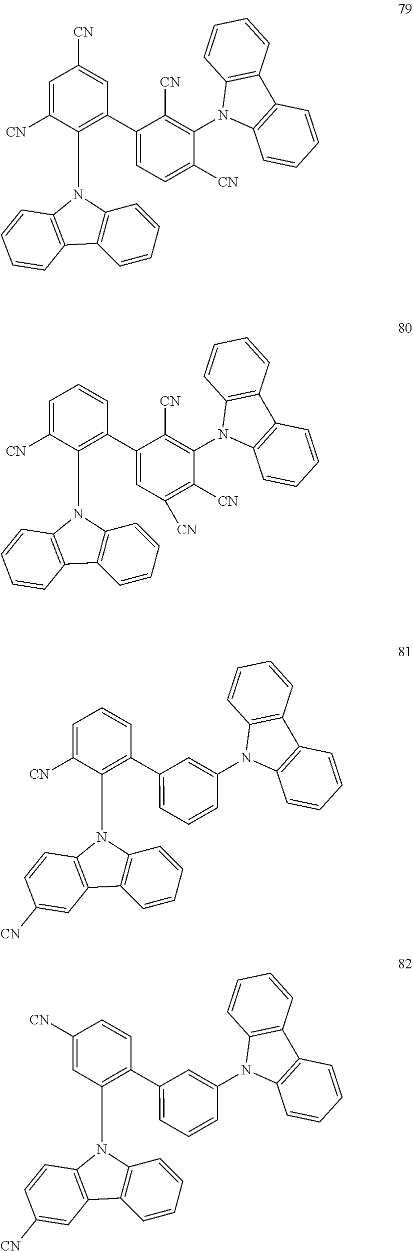

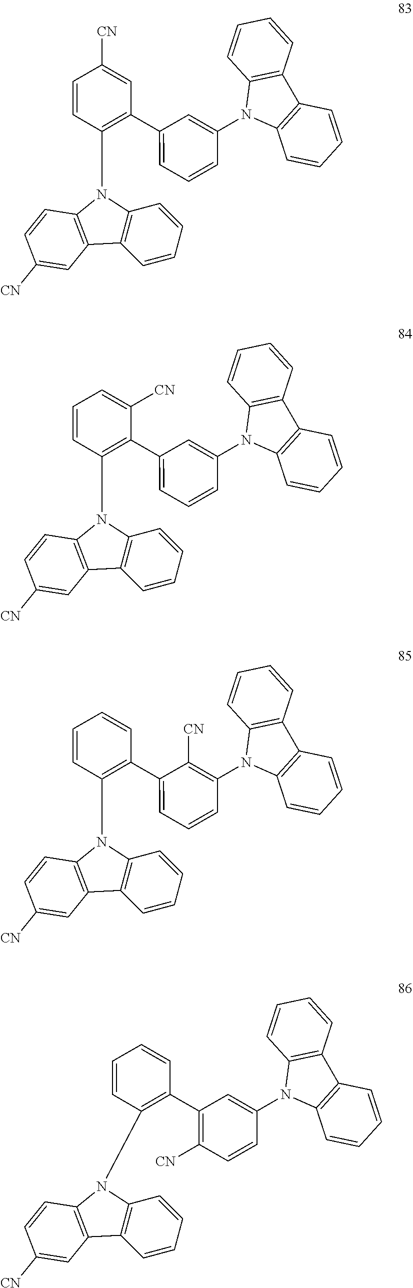

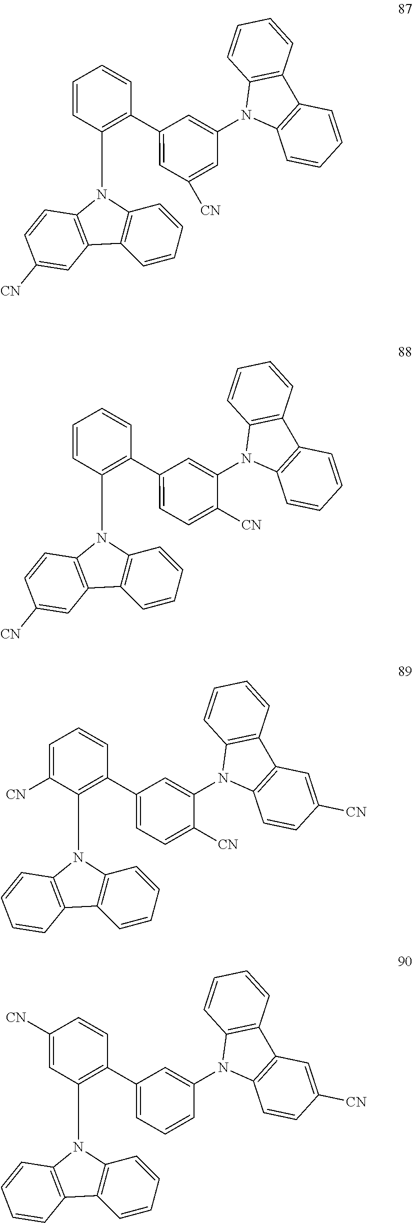

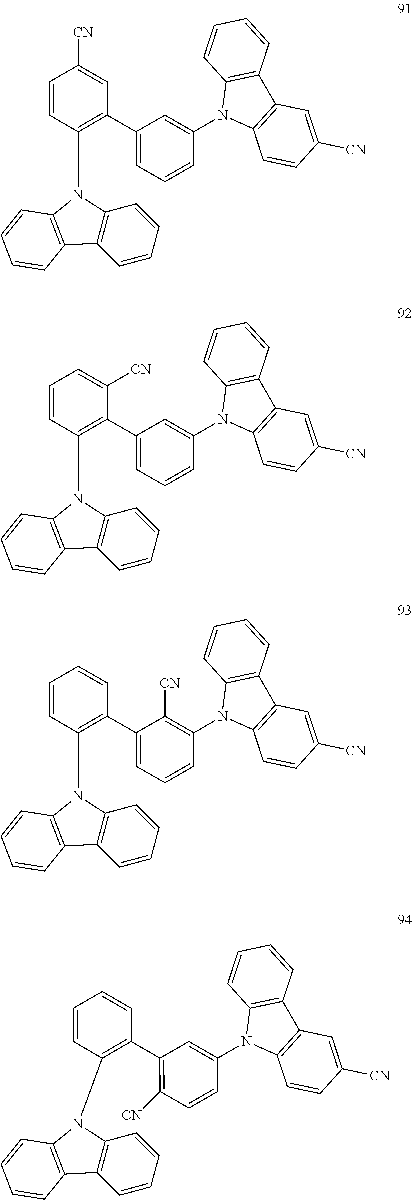

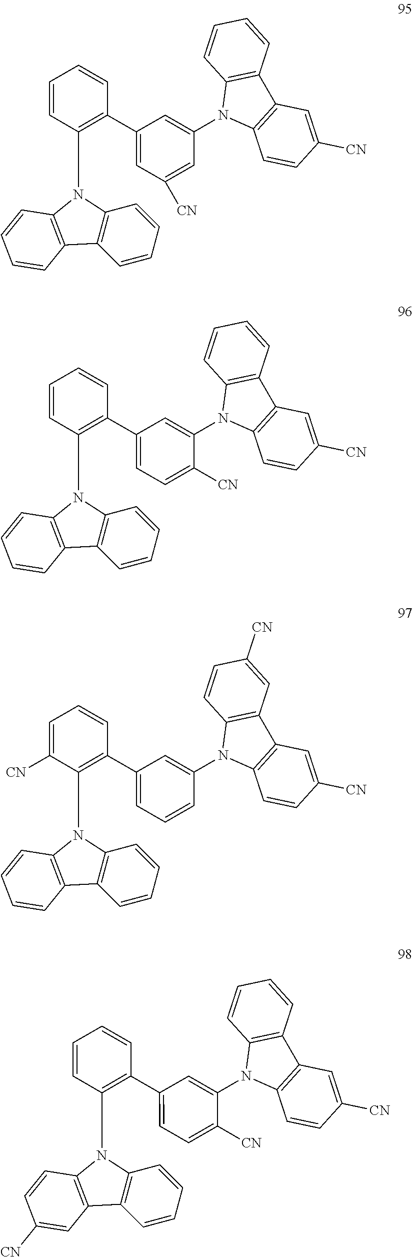

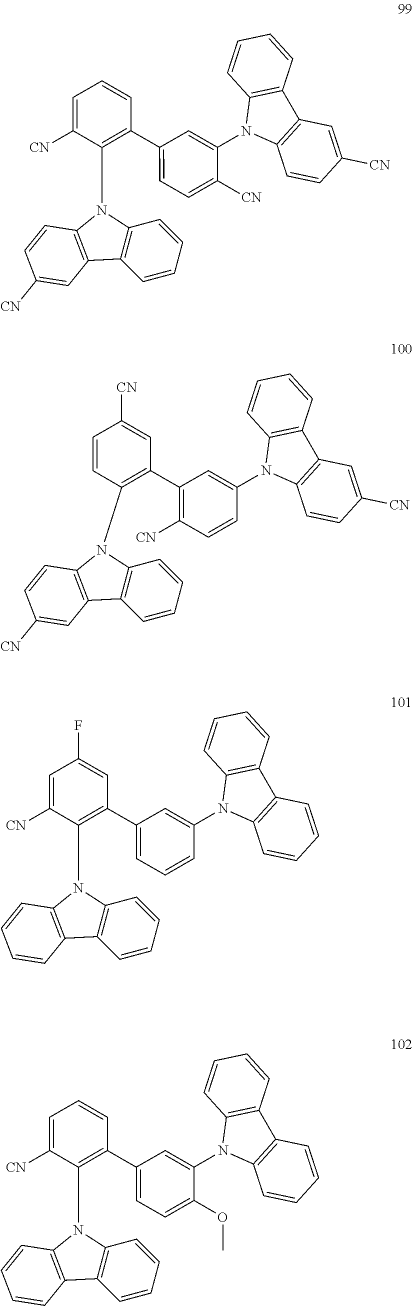

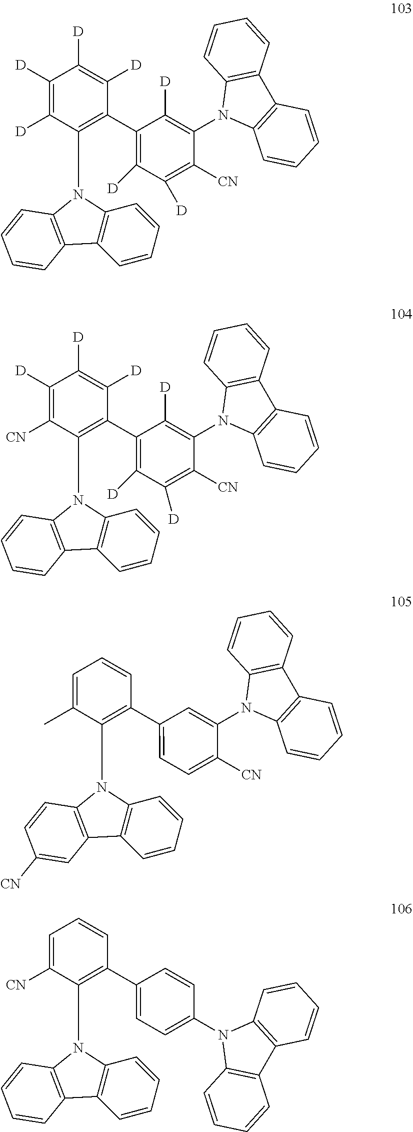

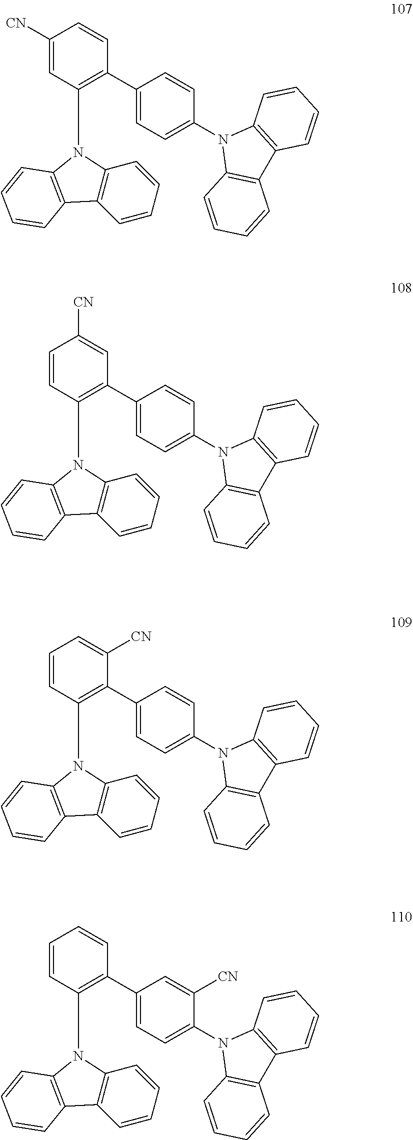

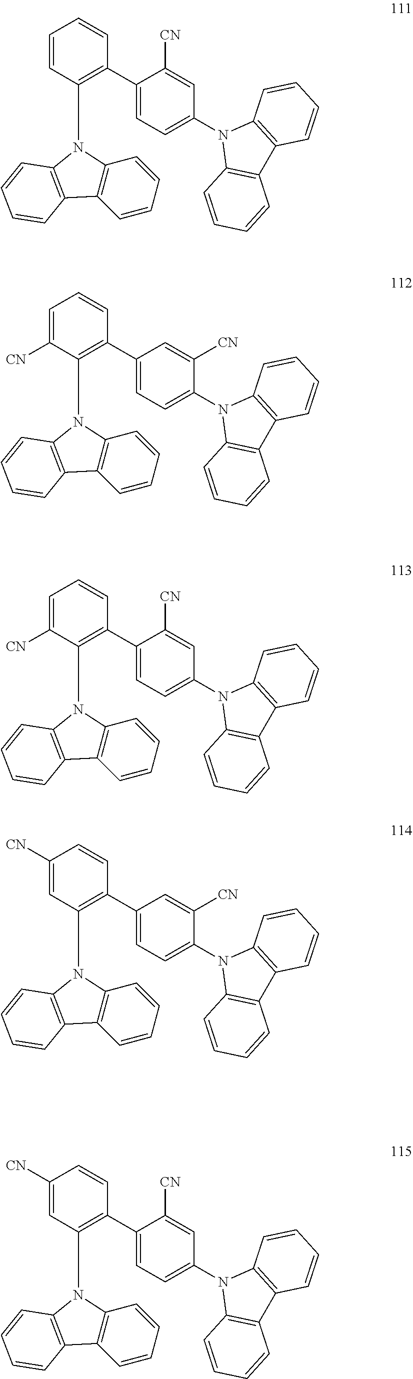

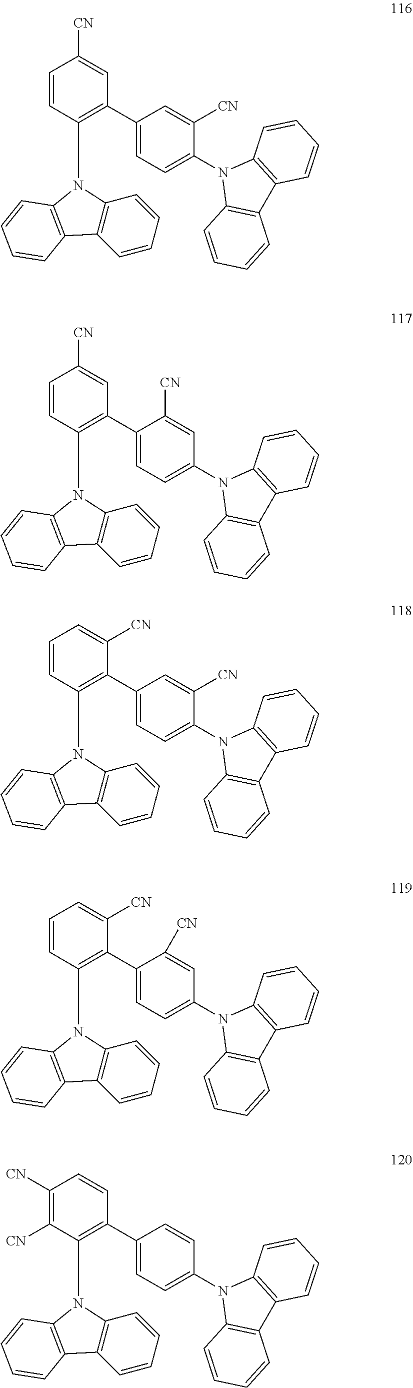

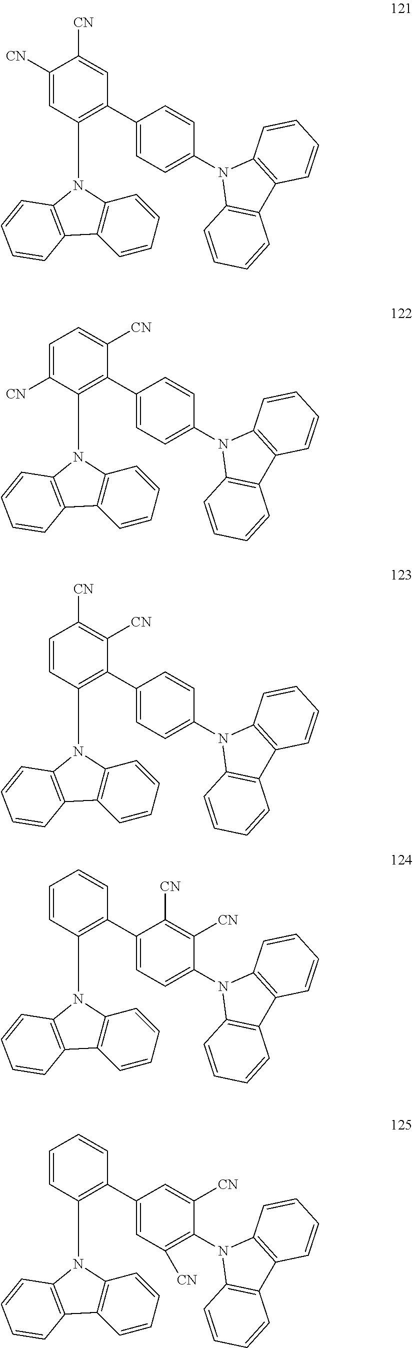

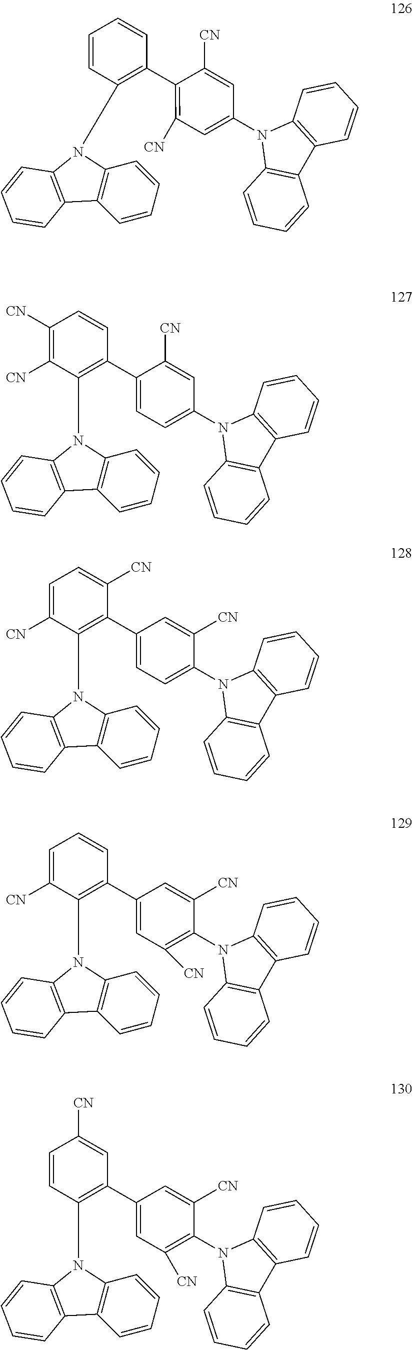

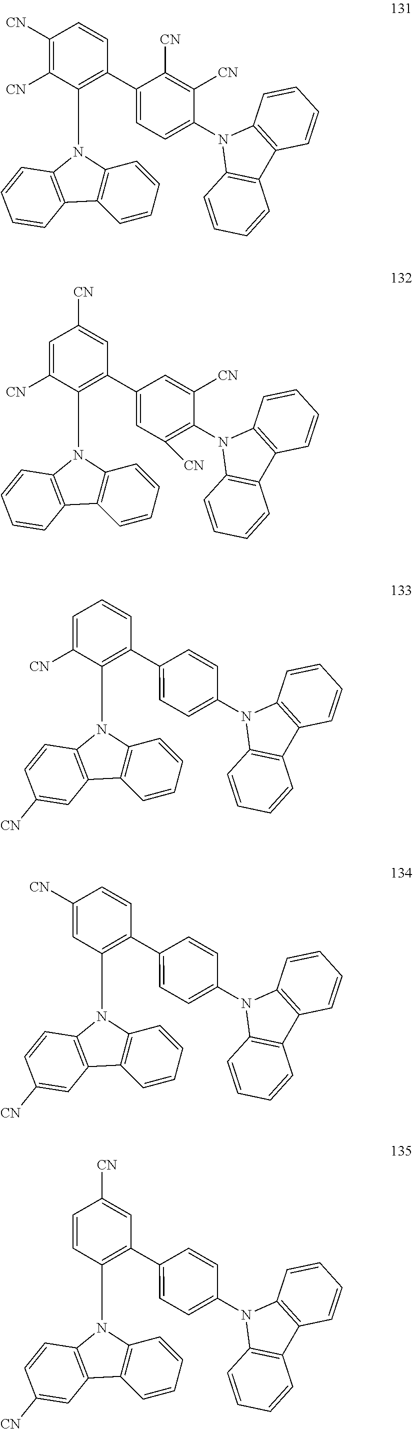

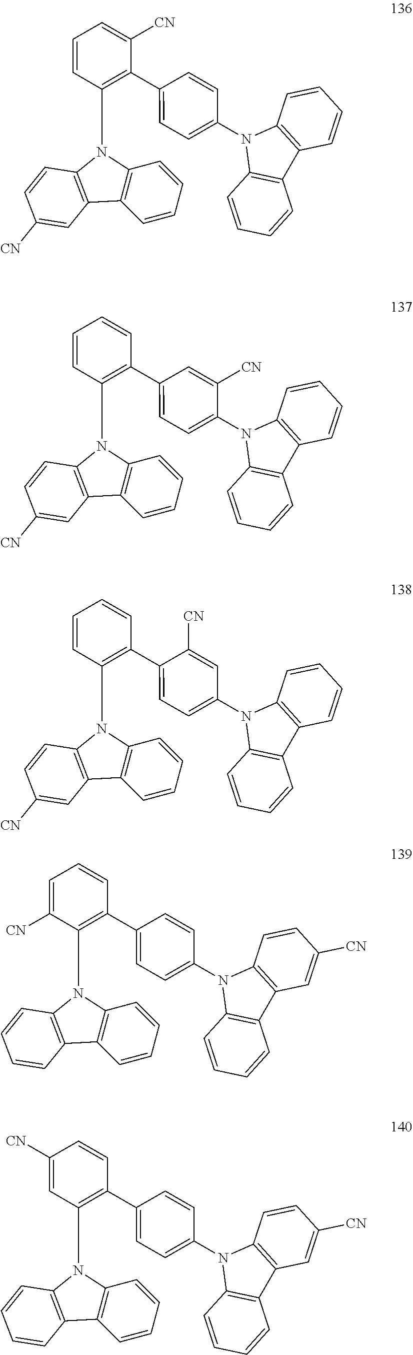

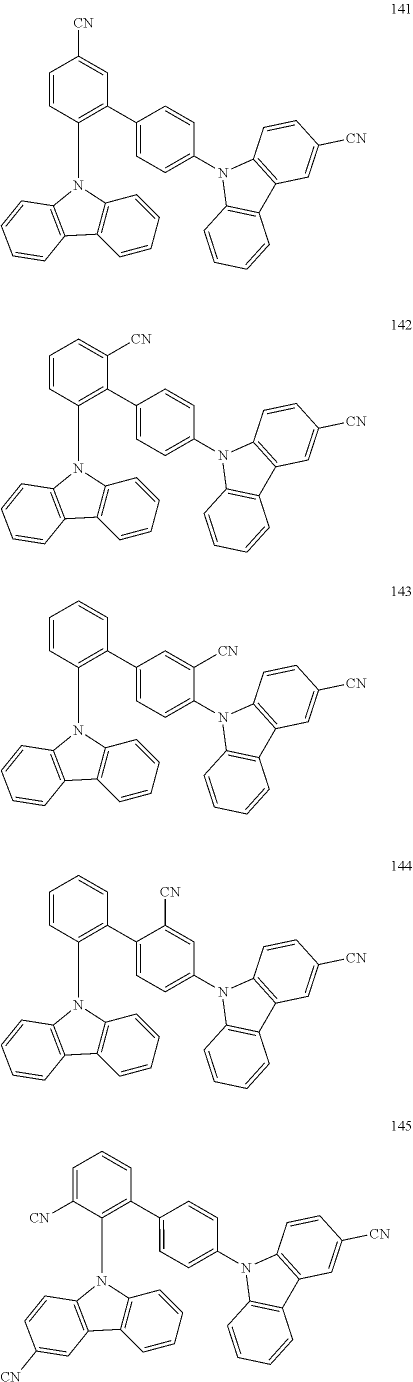

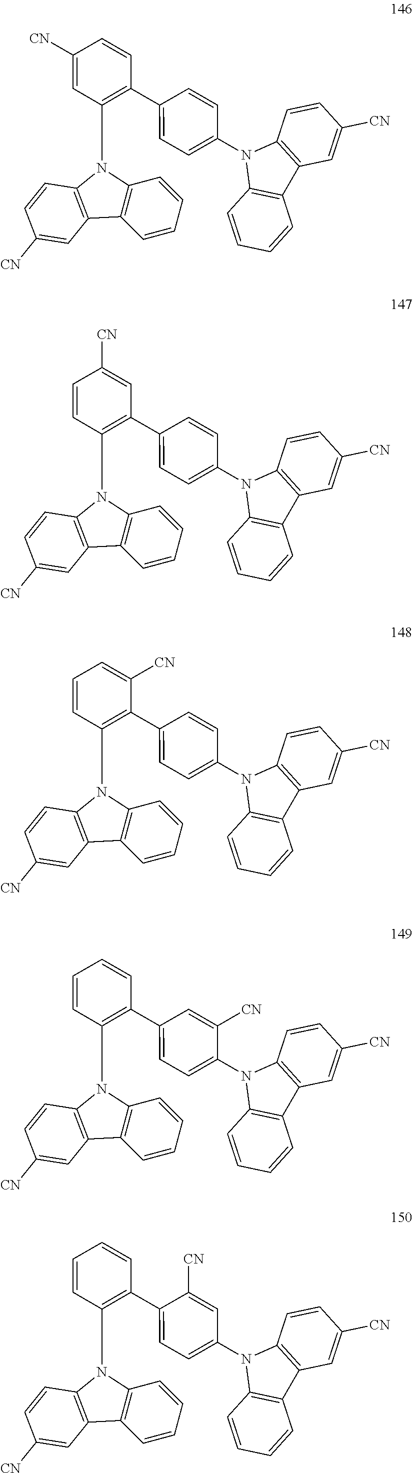

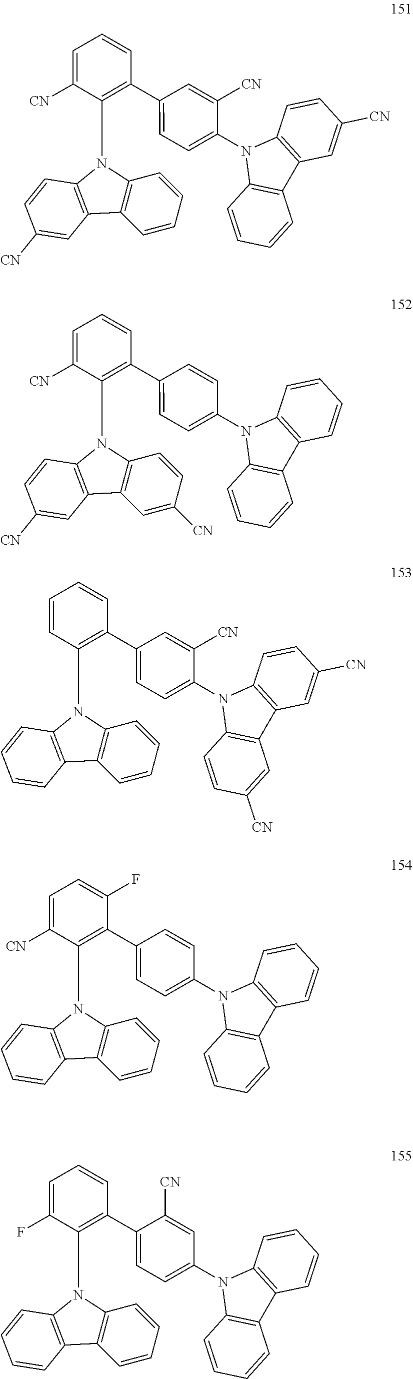

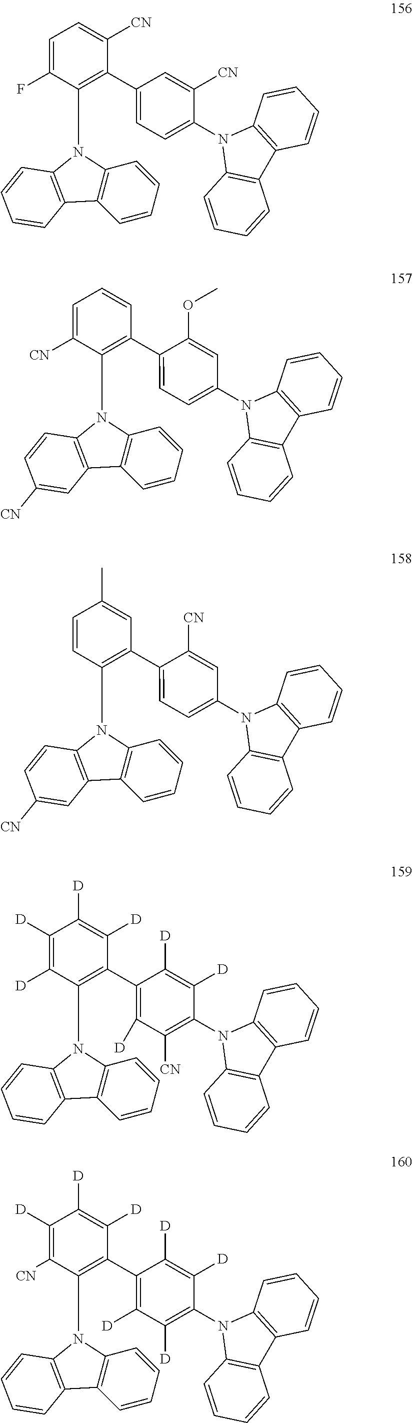

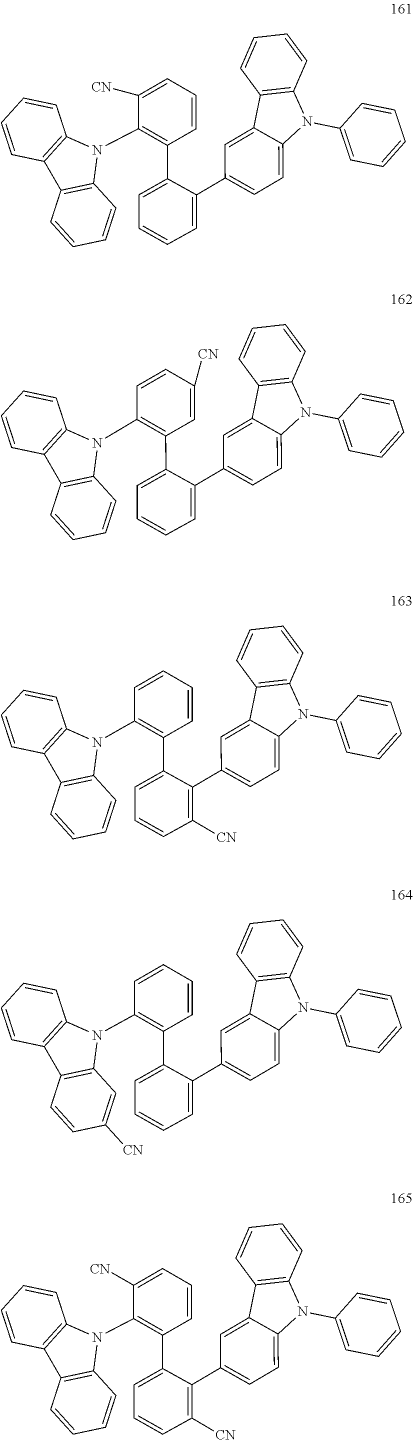

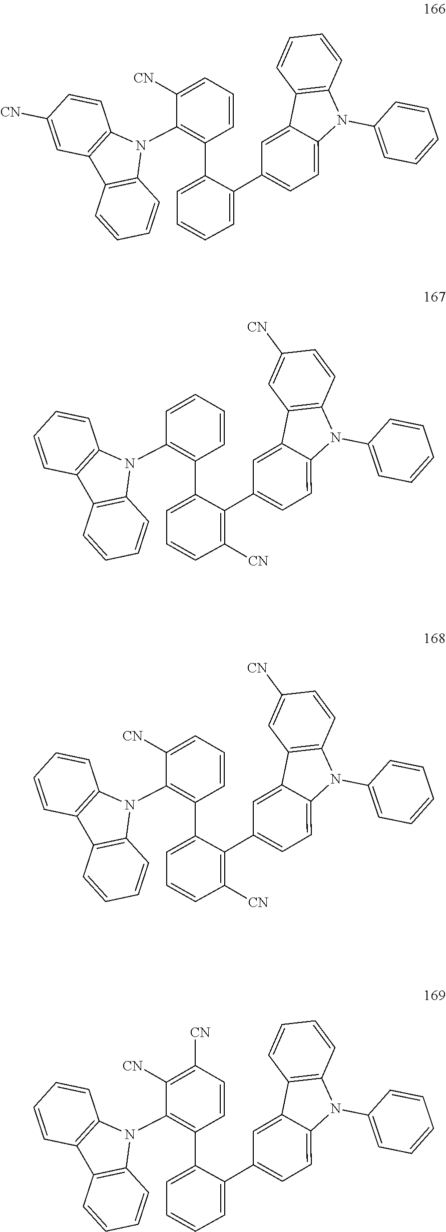

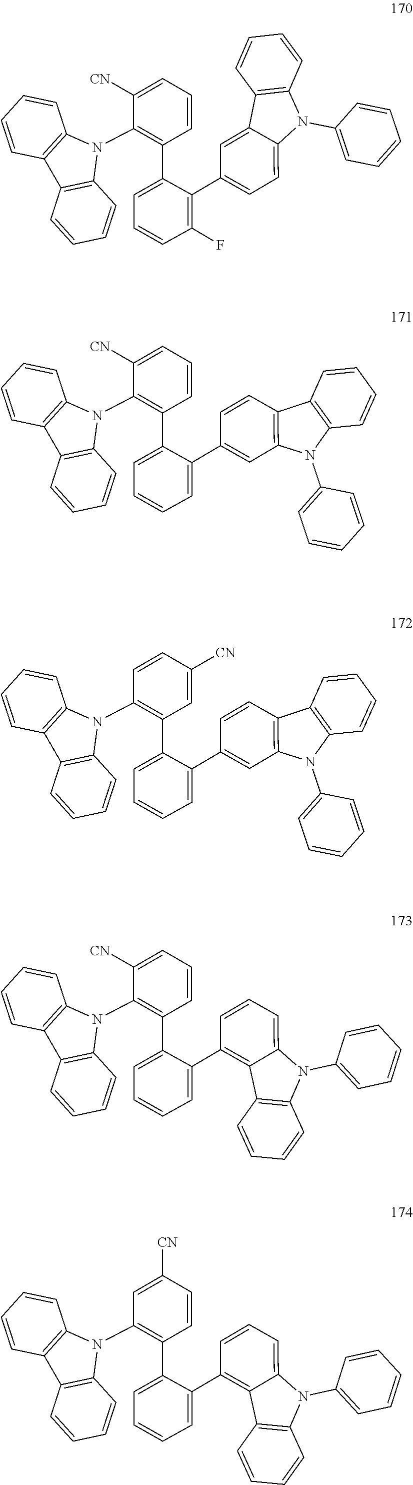

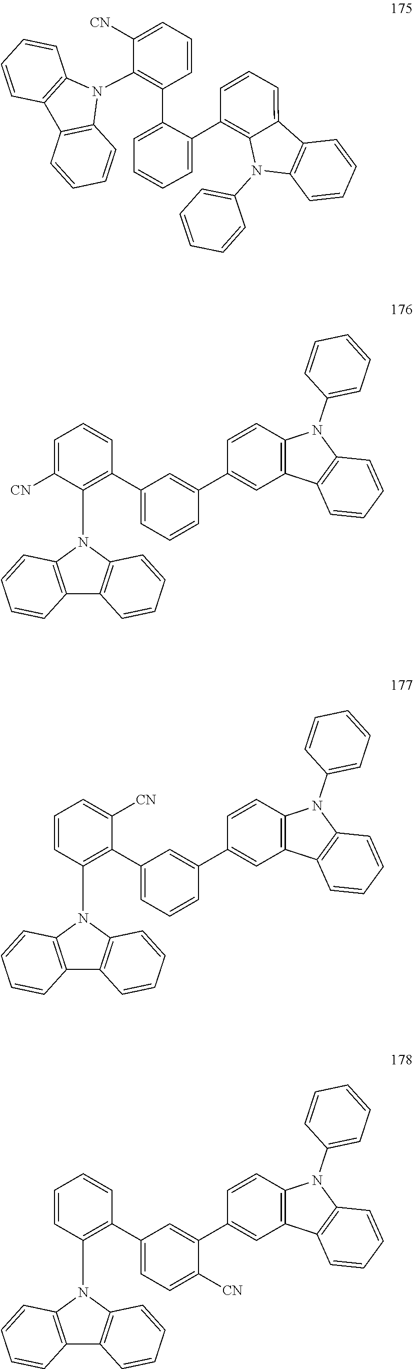

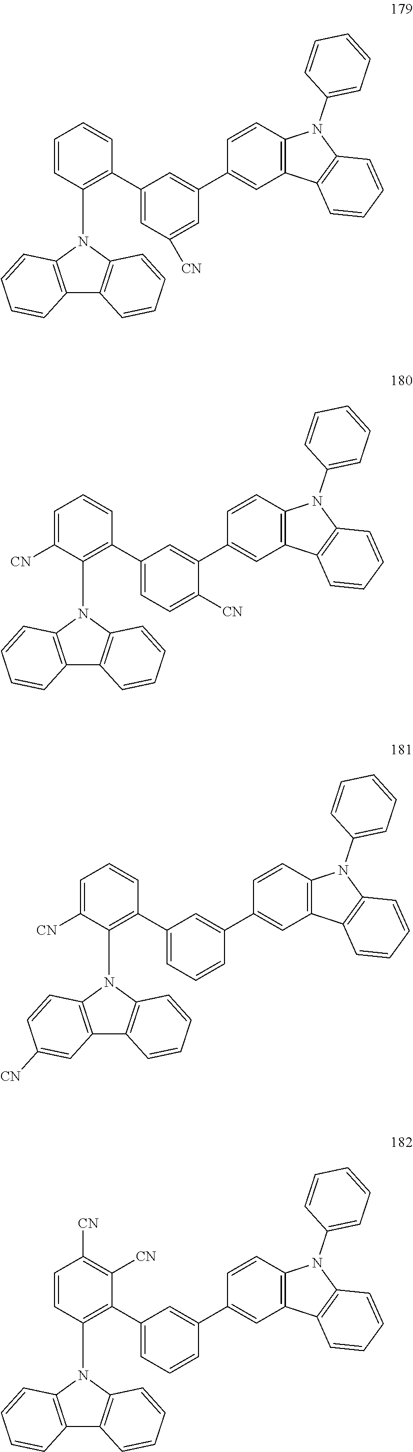

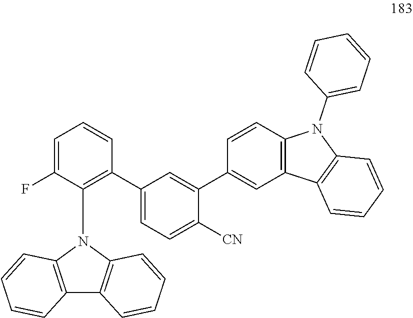 [SMILES: N#Cc1ccc(-c2cccc(F)c2-n2c3ccccc3c3ccccc32)cc1-c1ccc2c(c1)c1ccccc1n2-c1ccccc1]

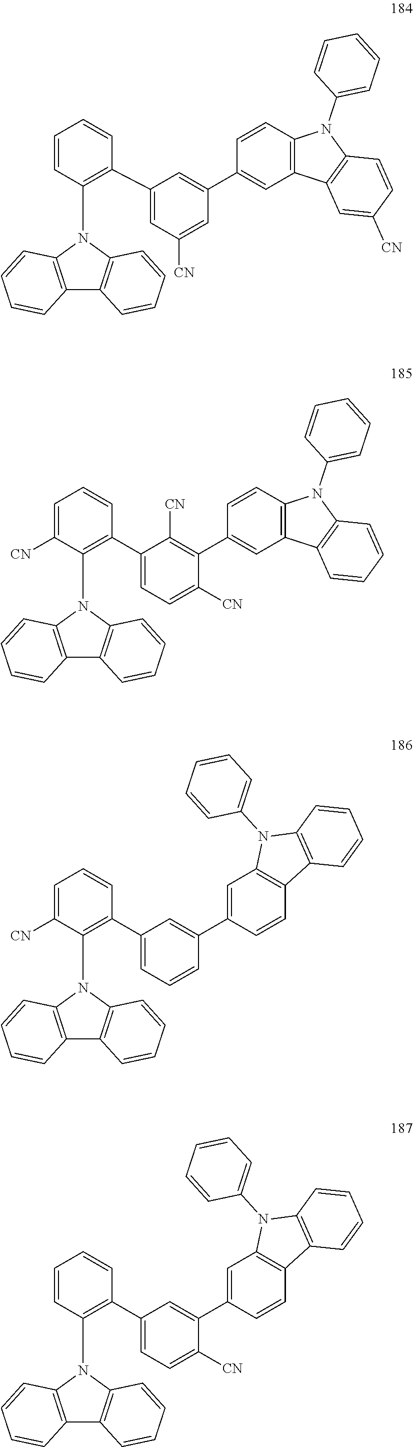

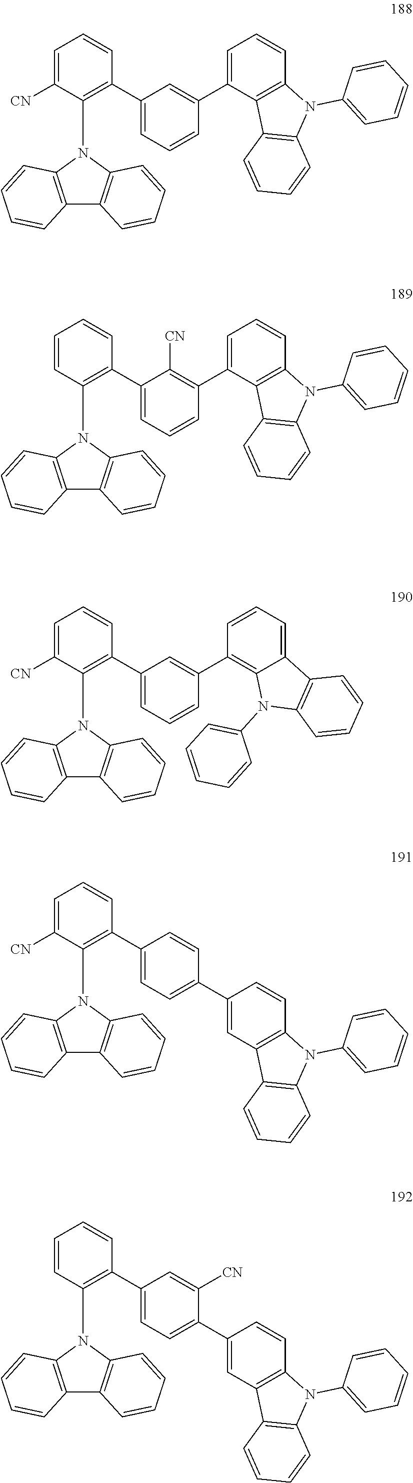

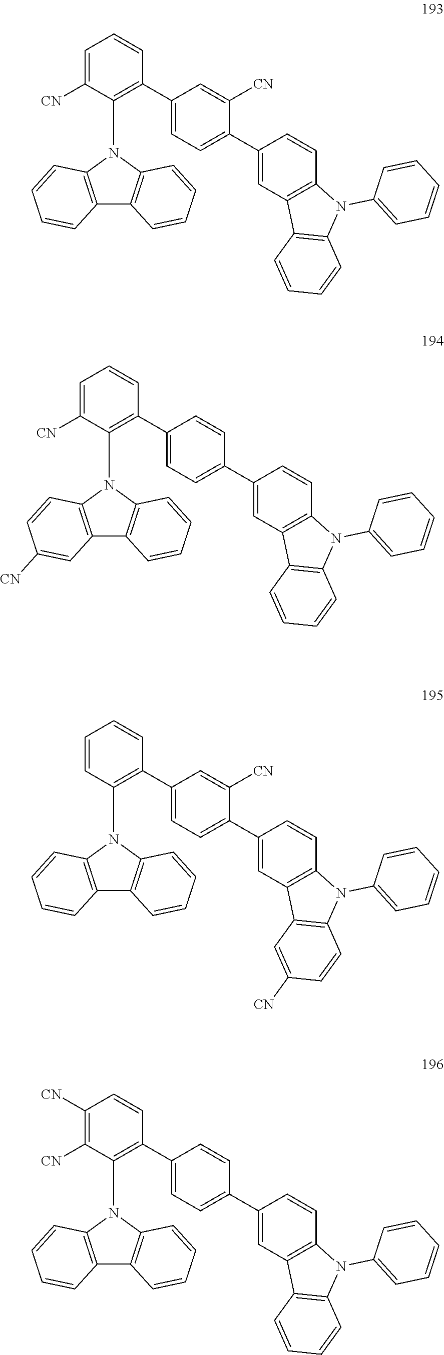

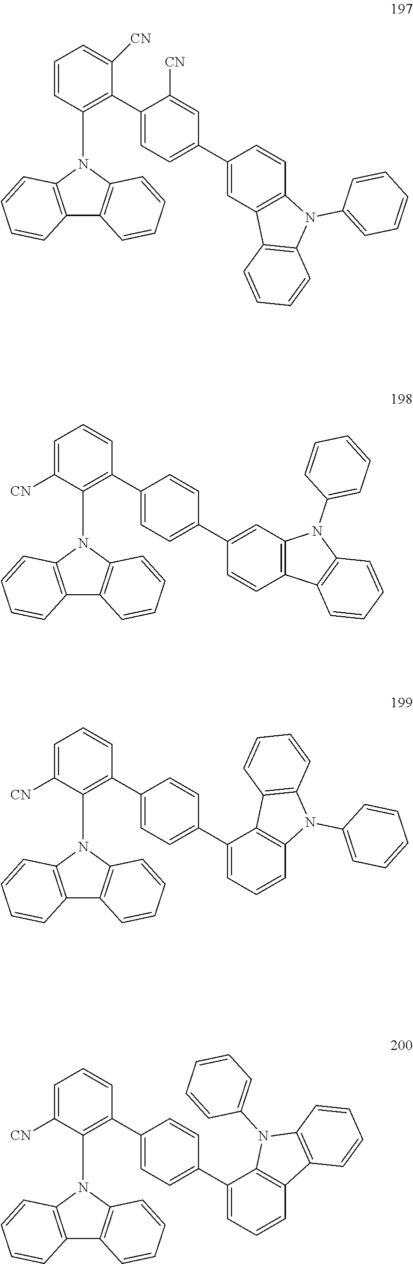

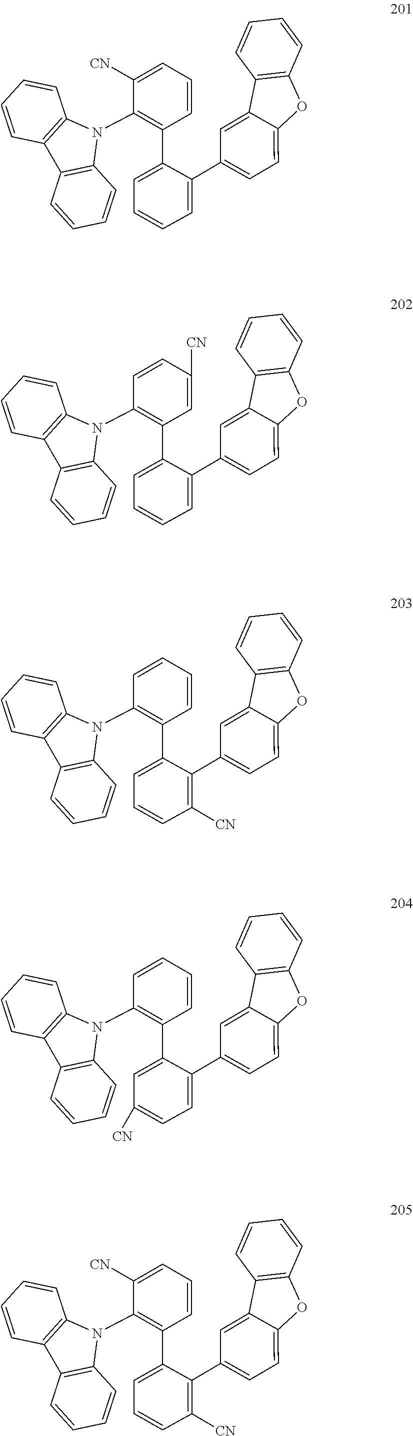

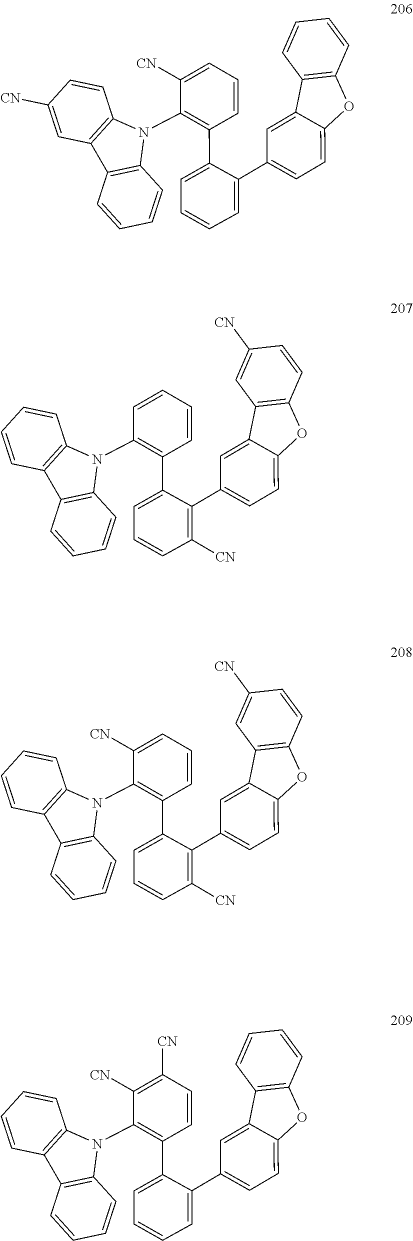

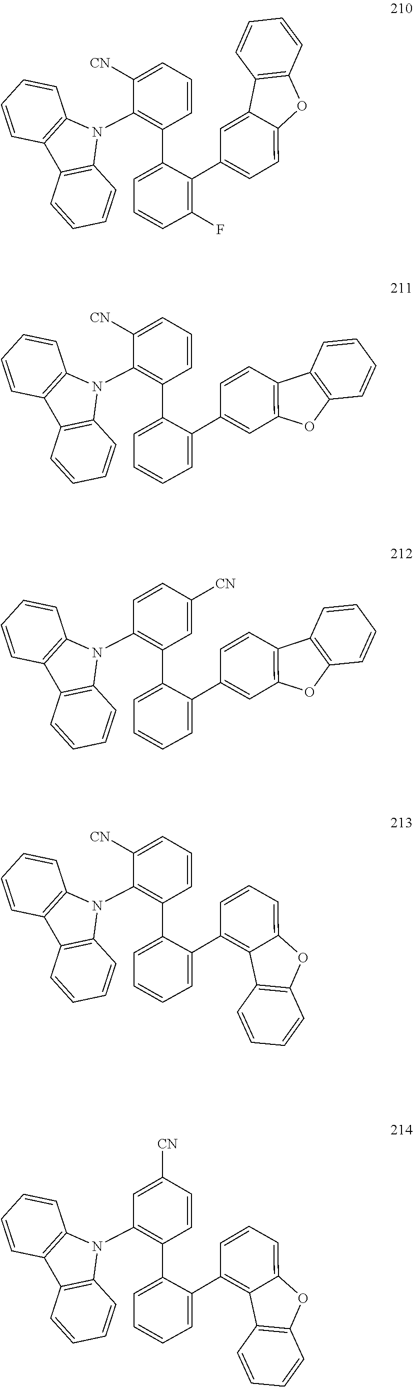

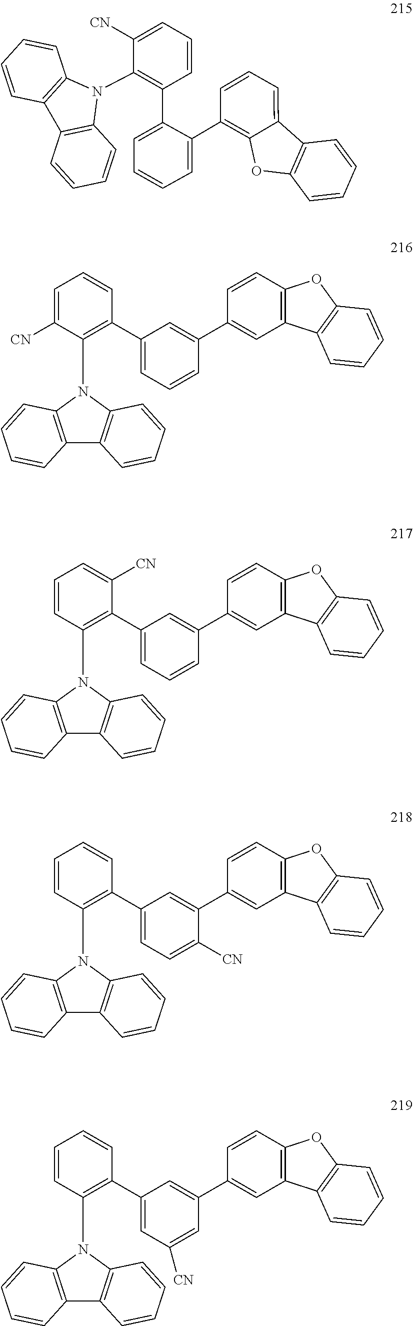

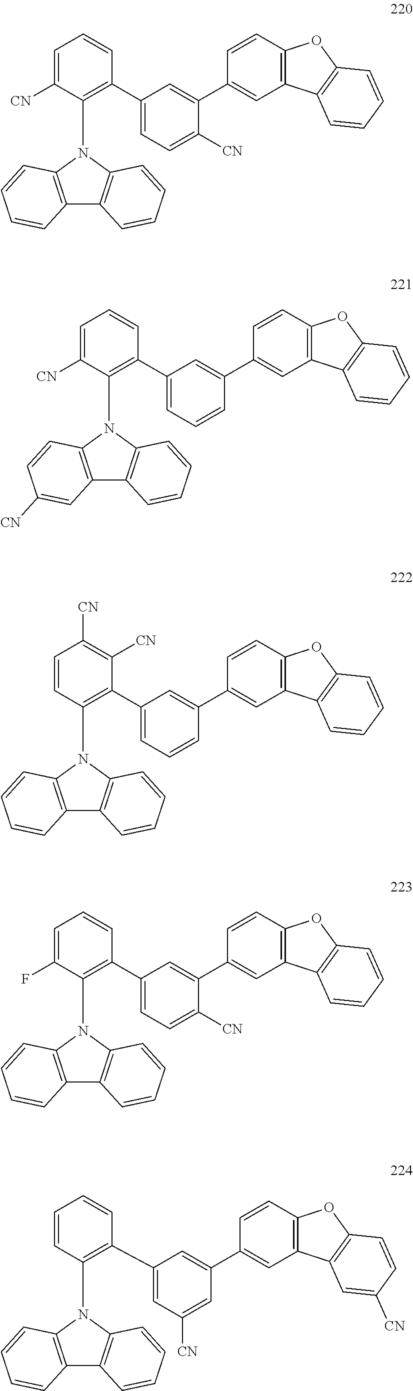

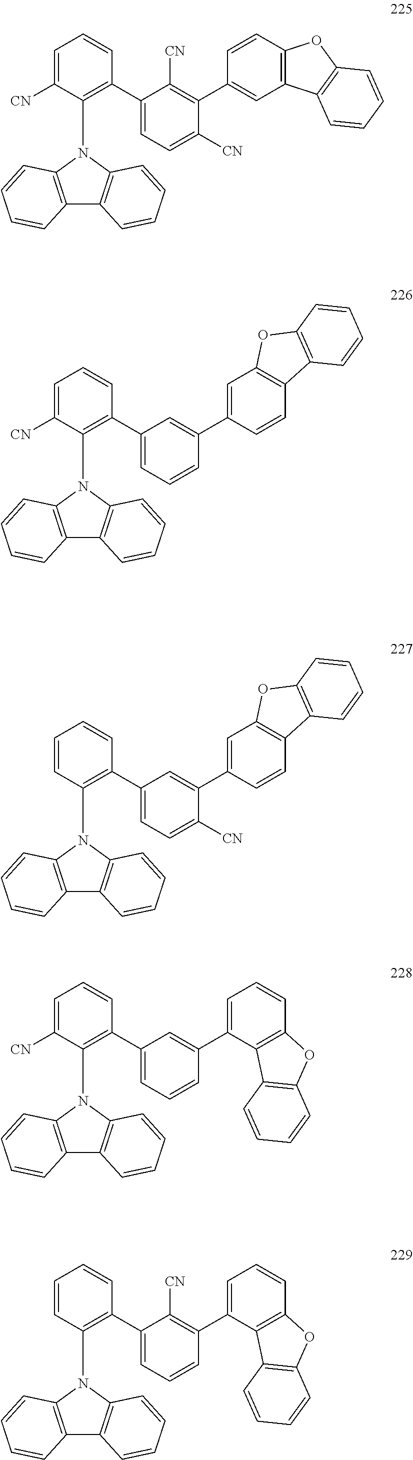

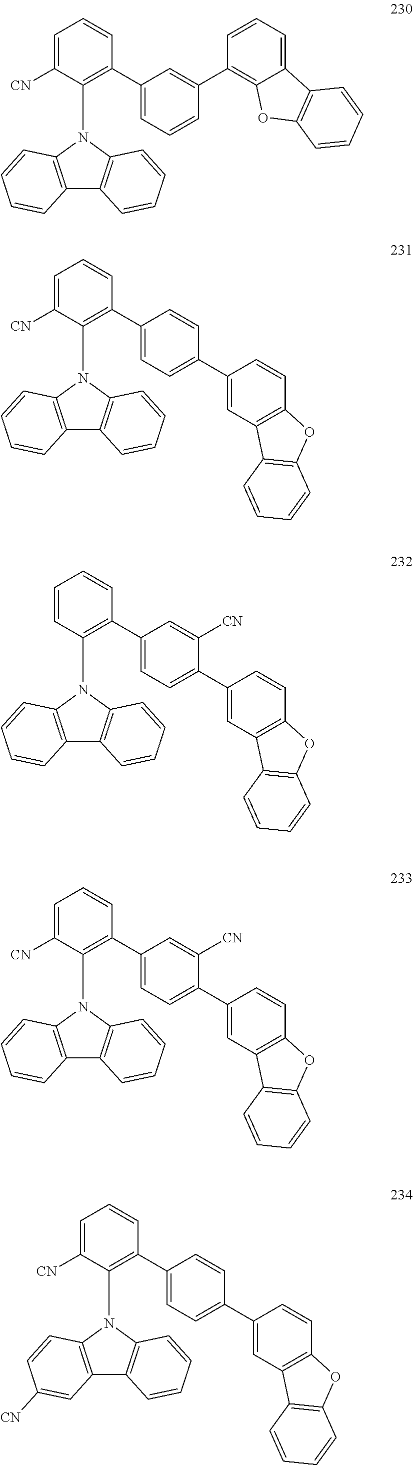

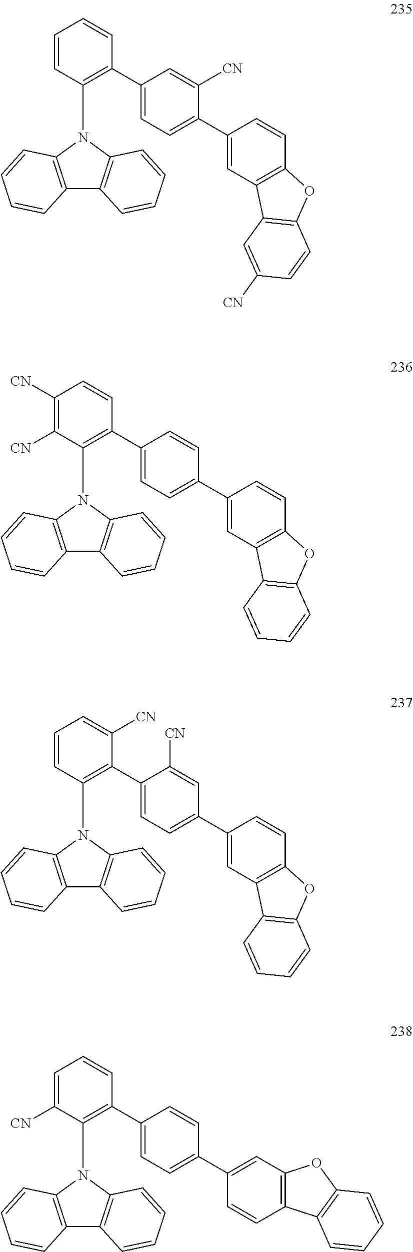

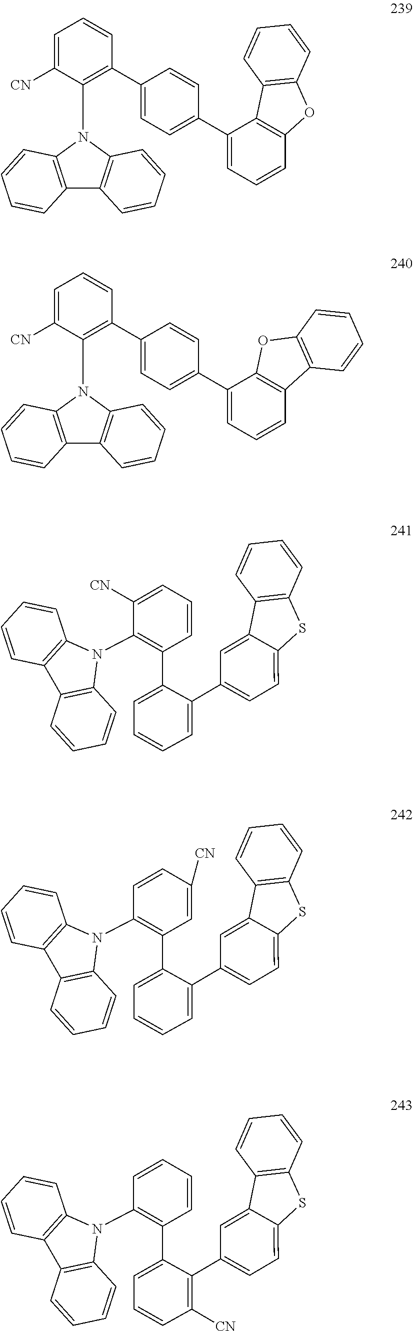

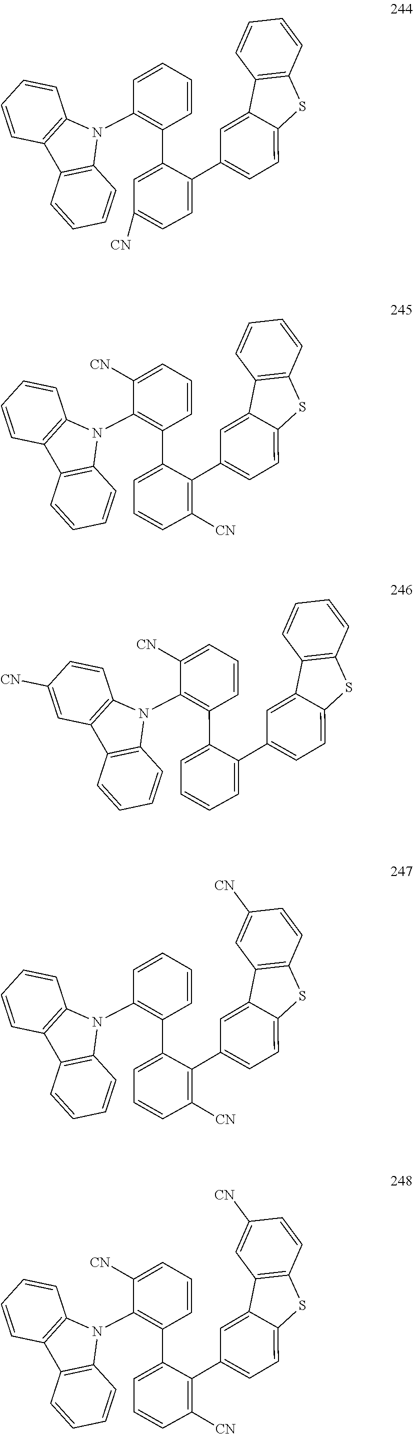

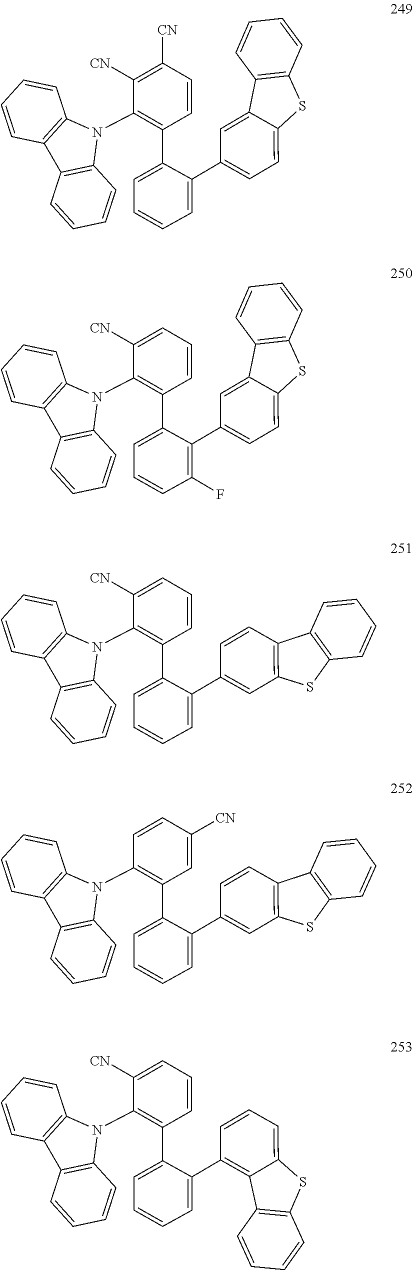

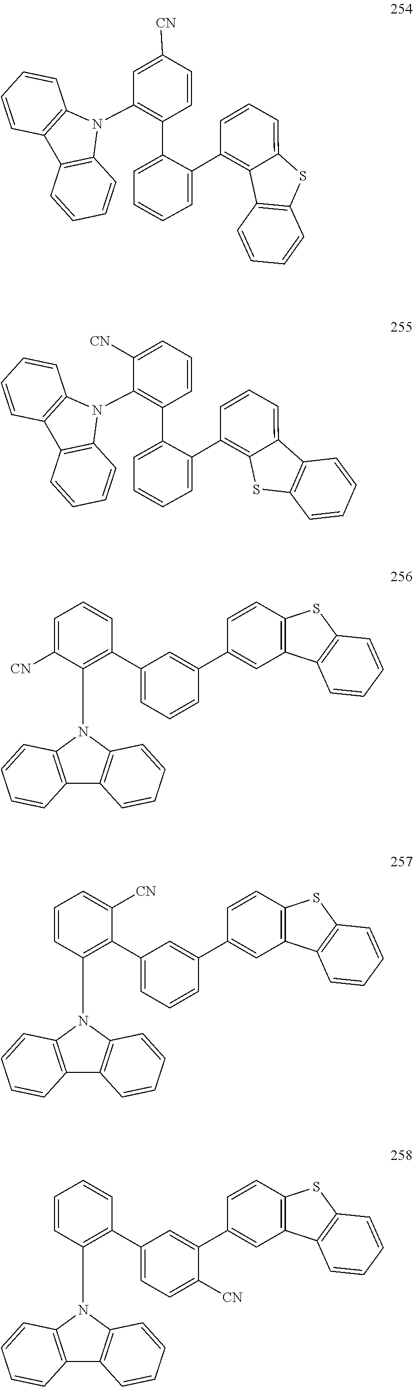

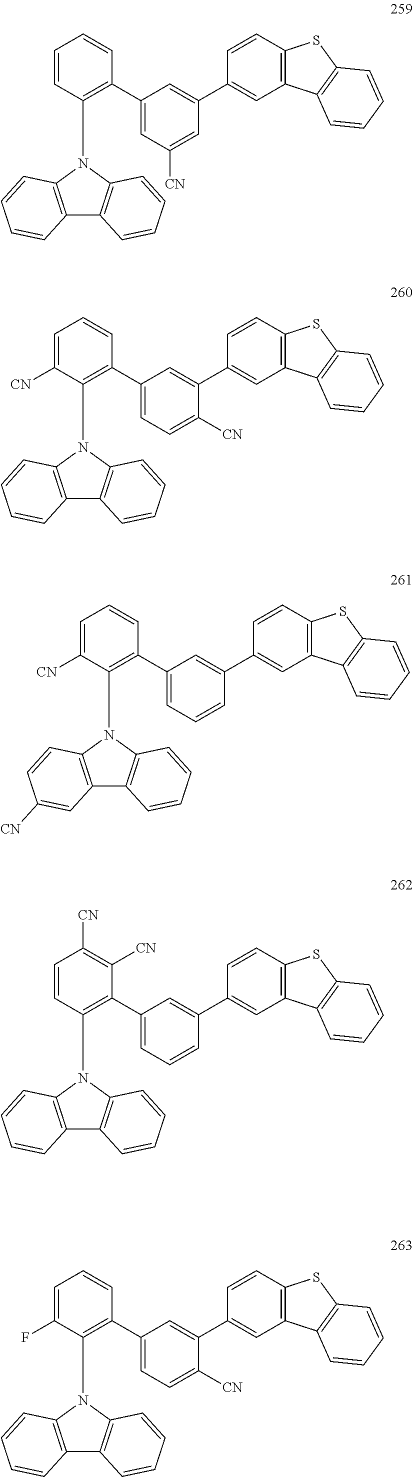

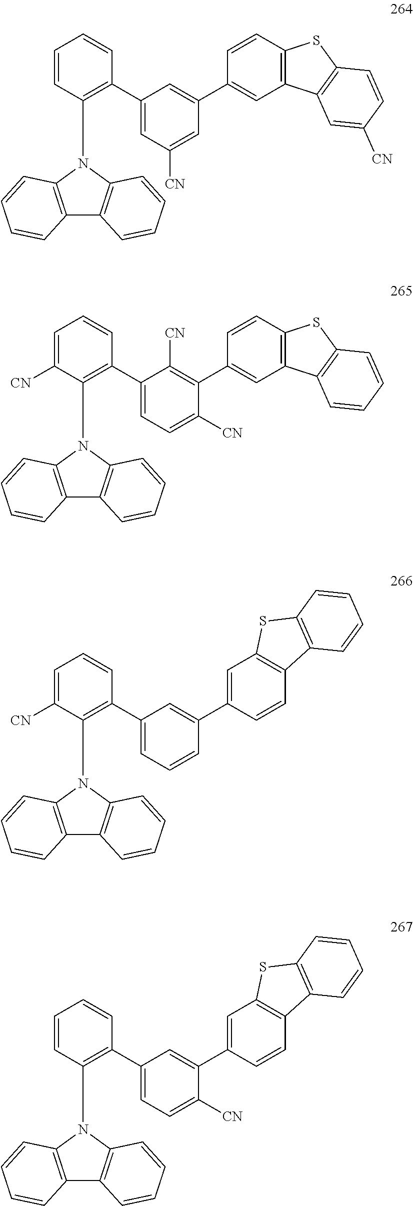

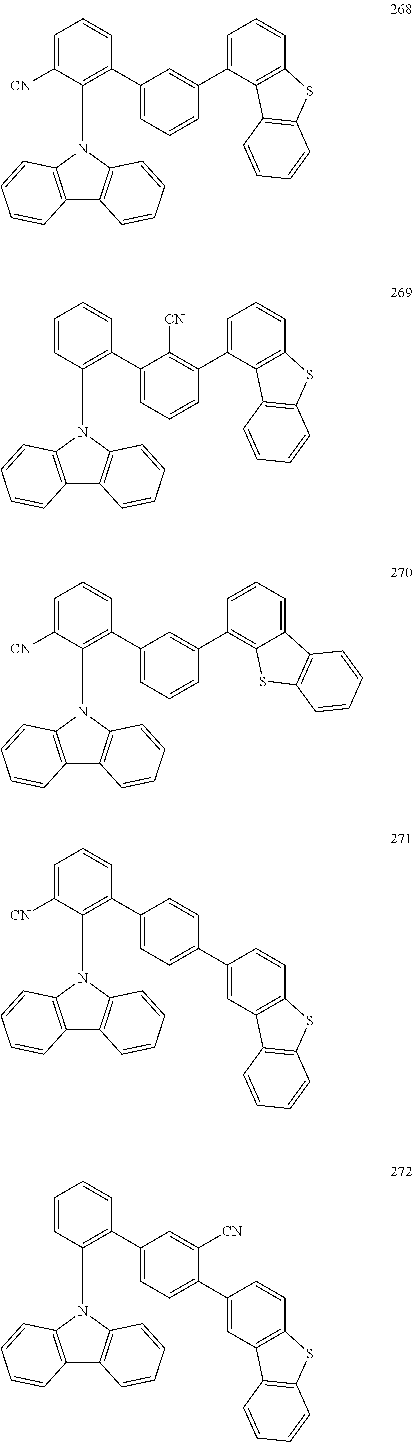

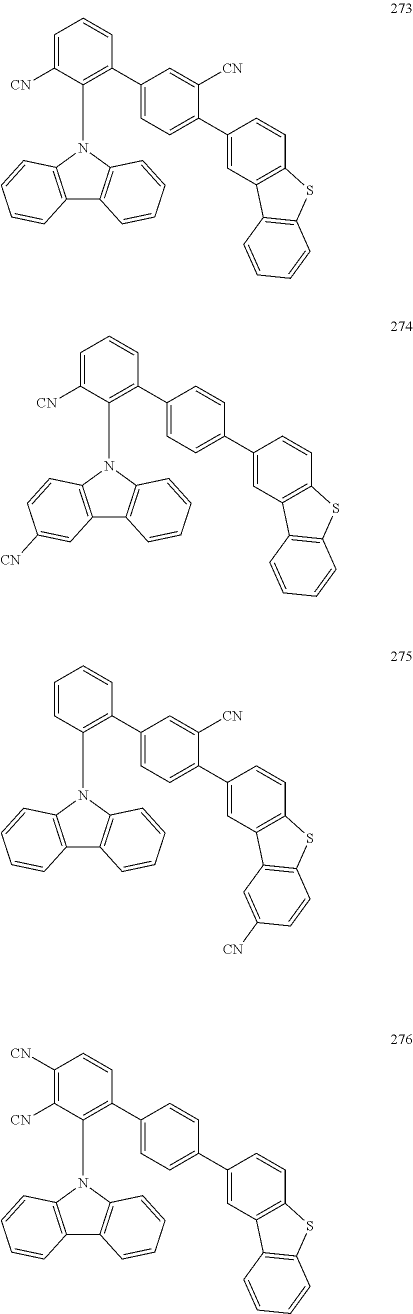

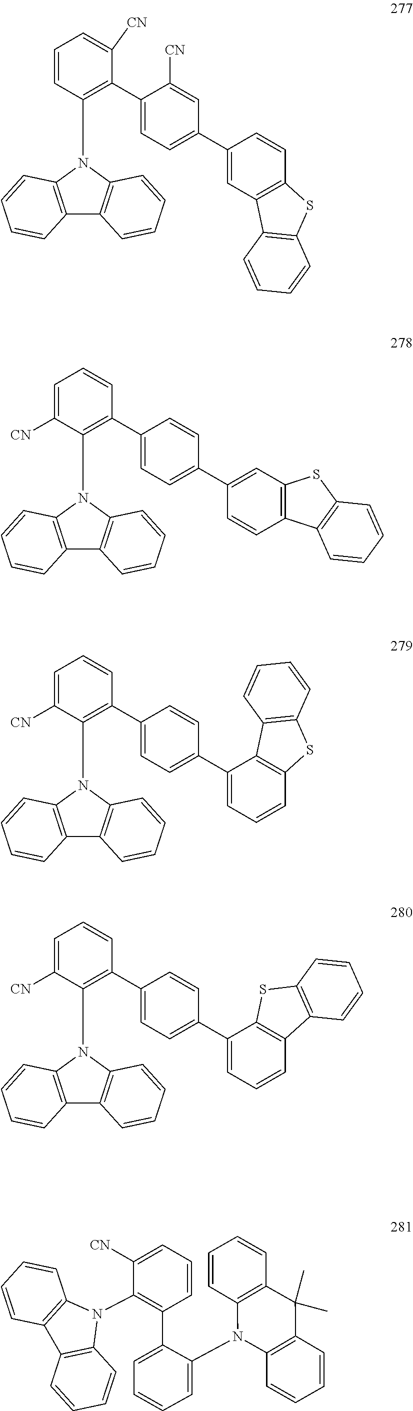

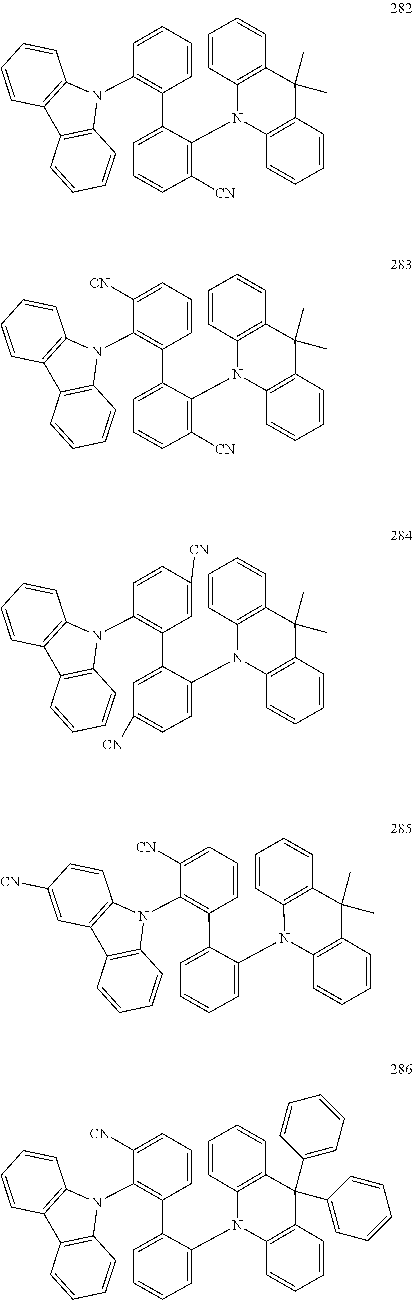

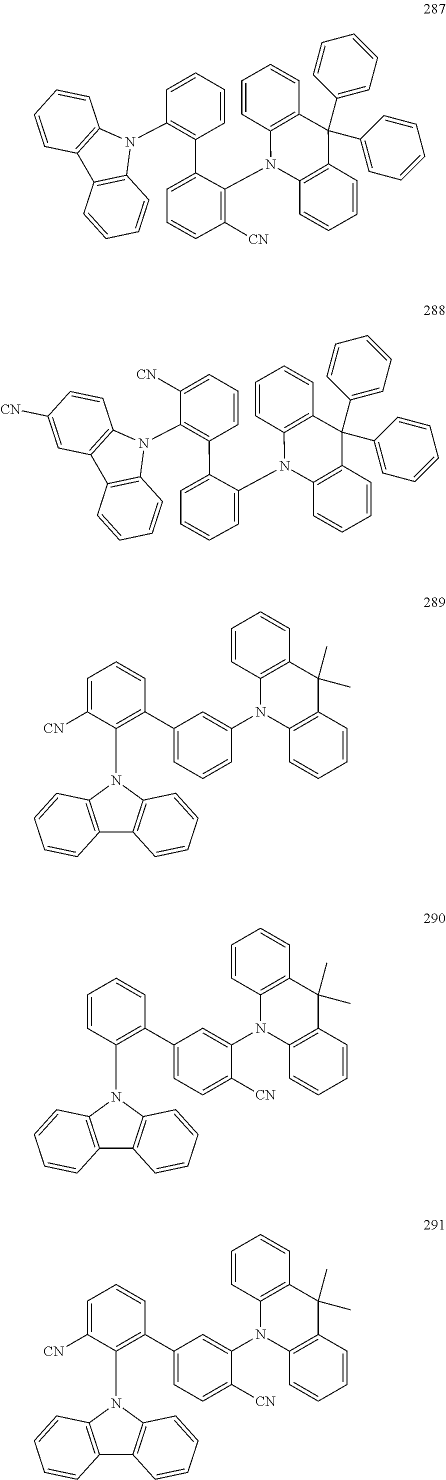

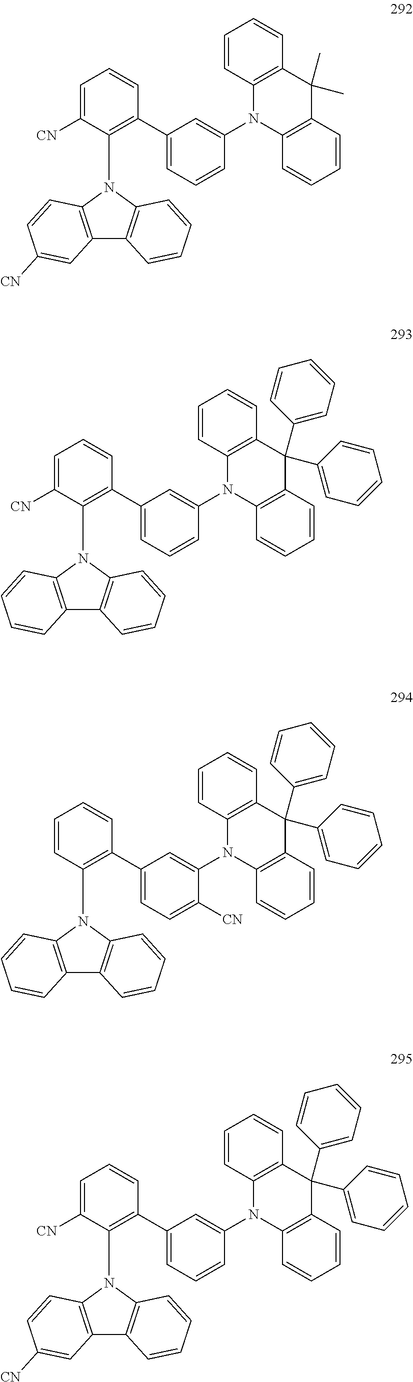

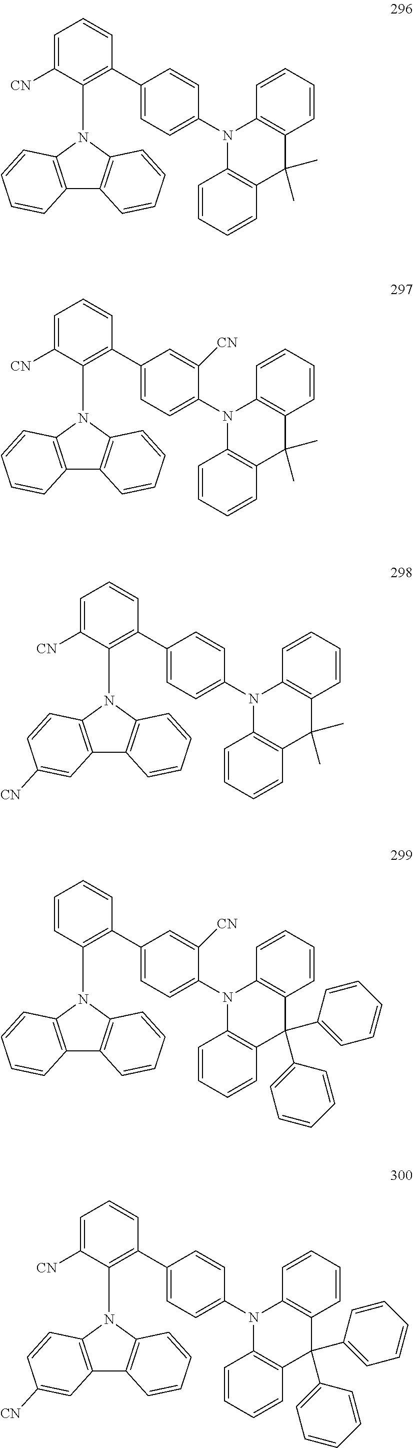

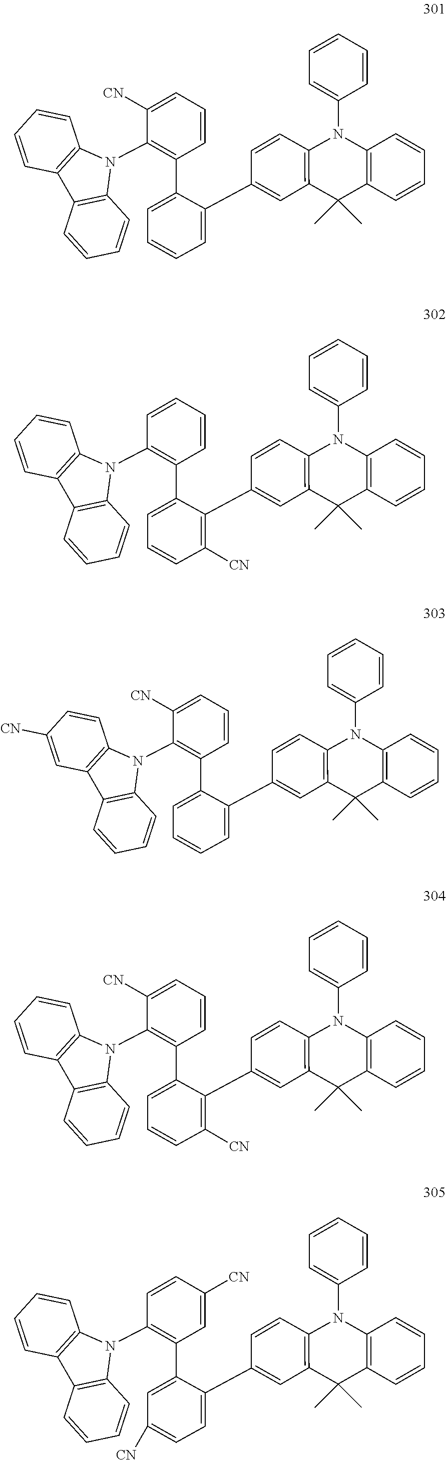

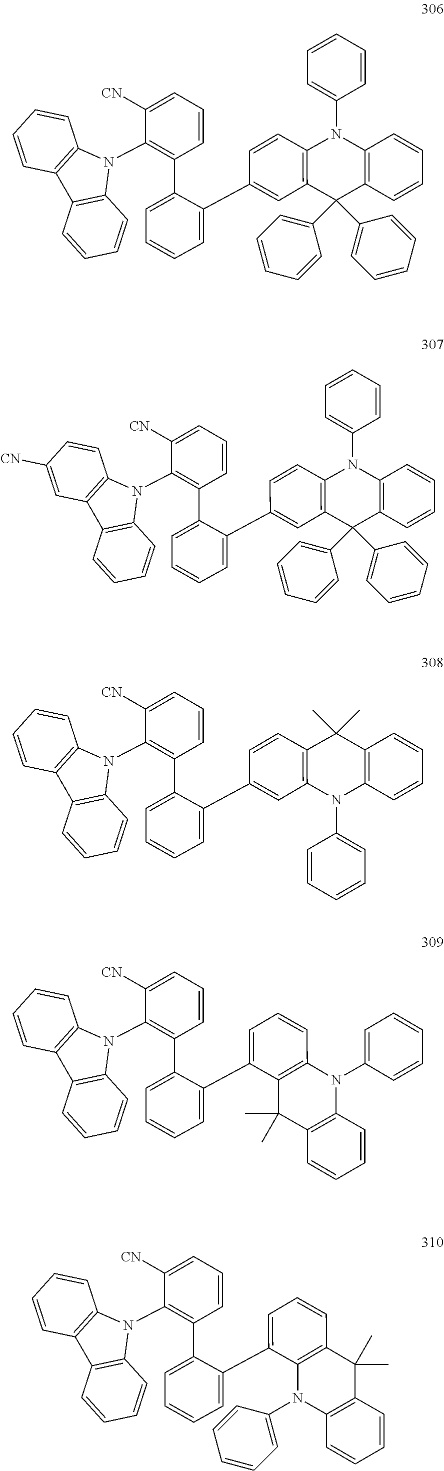

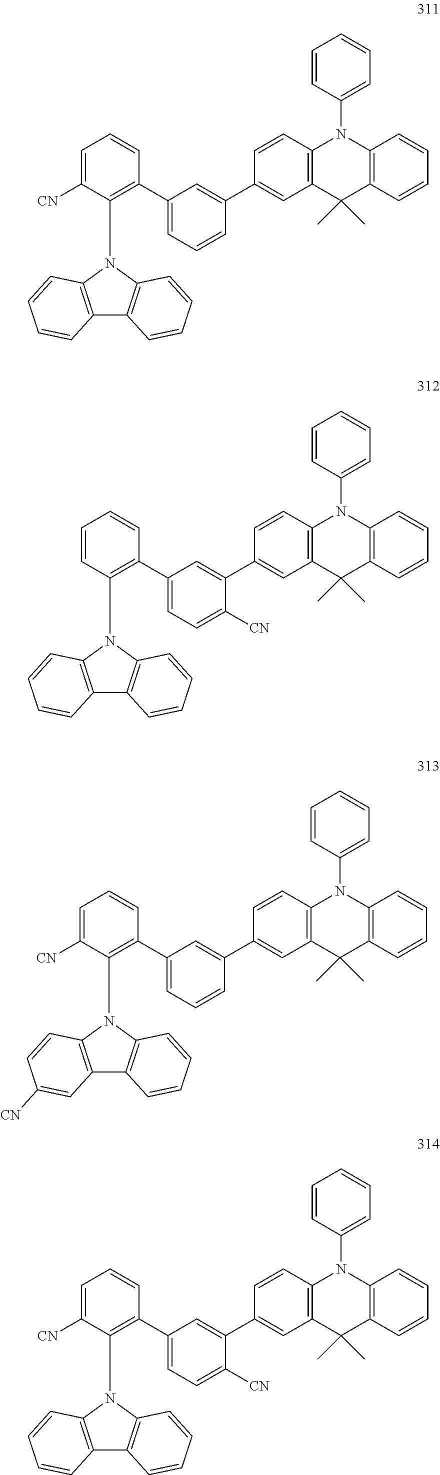

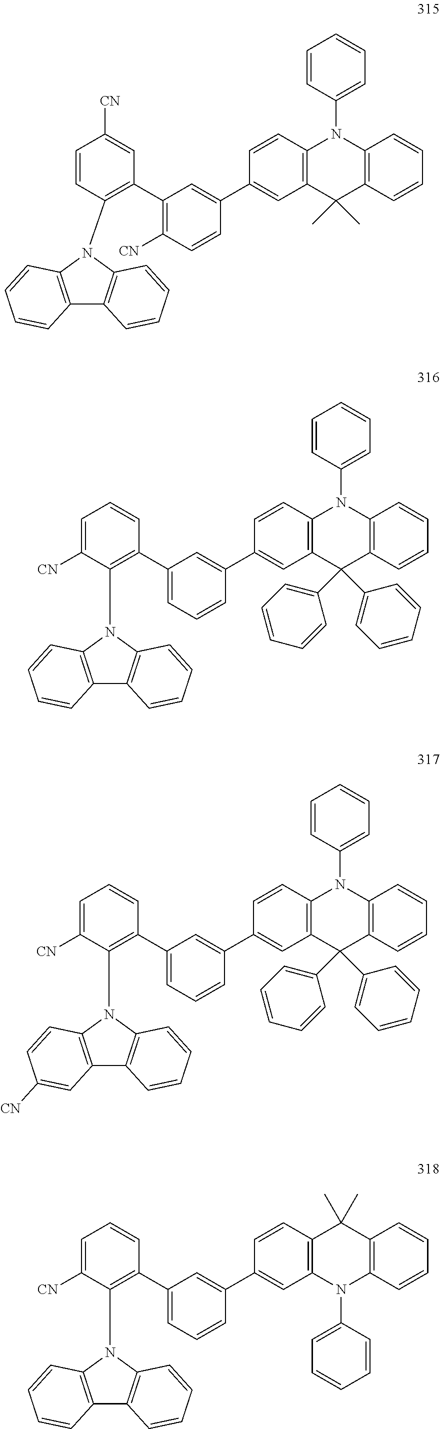

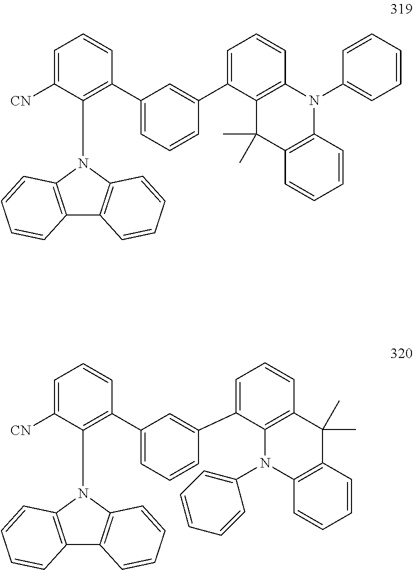

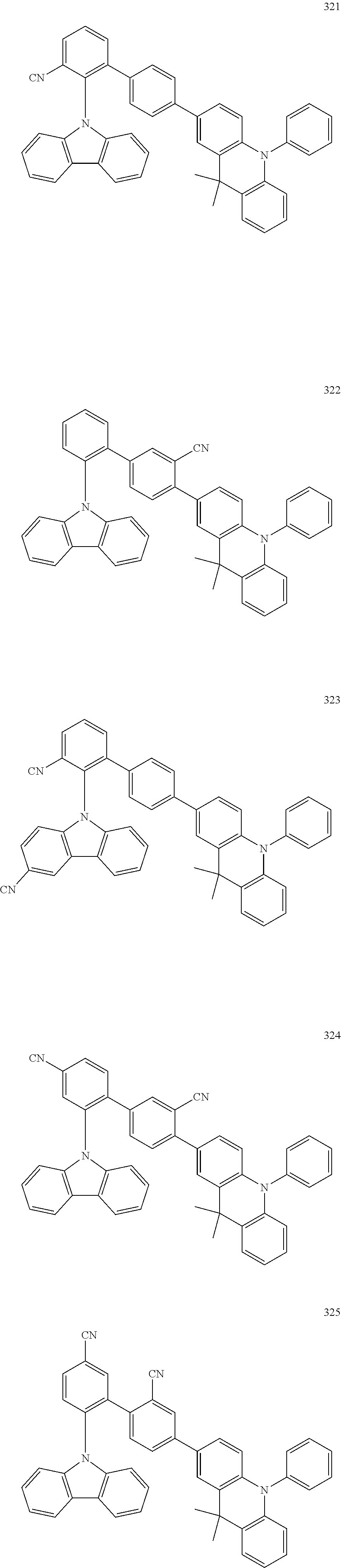

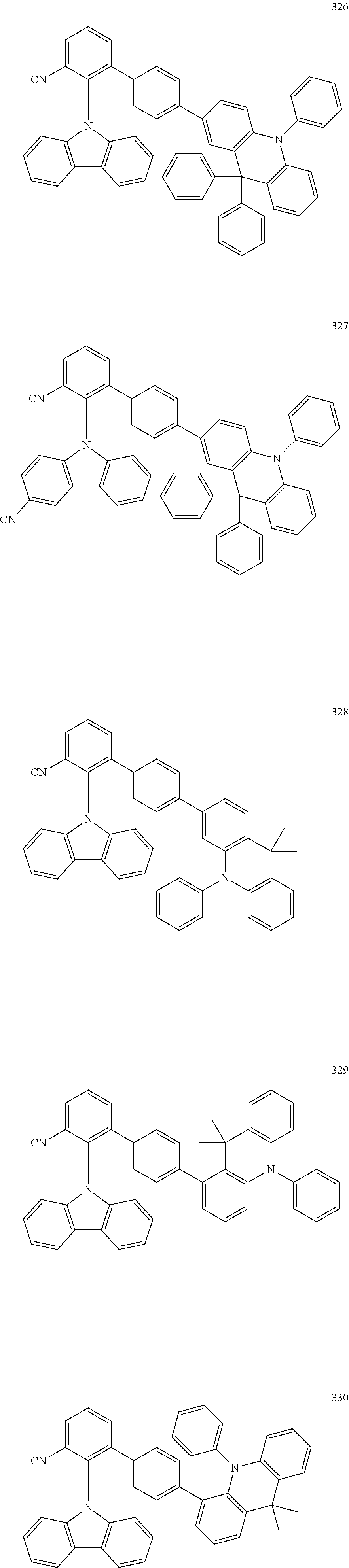

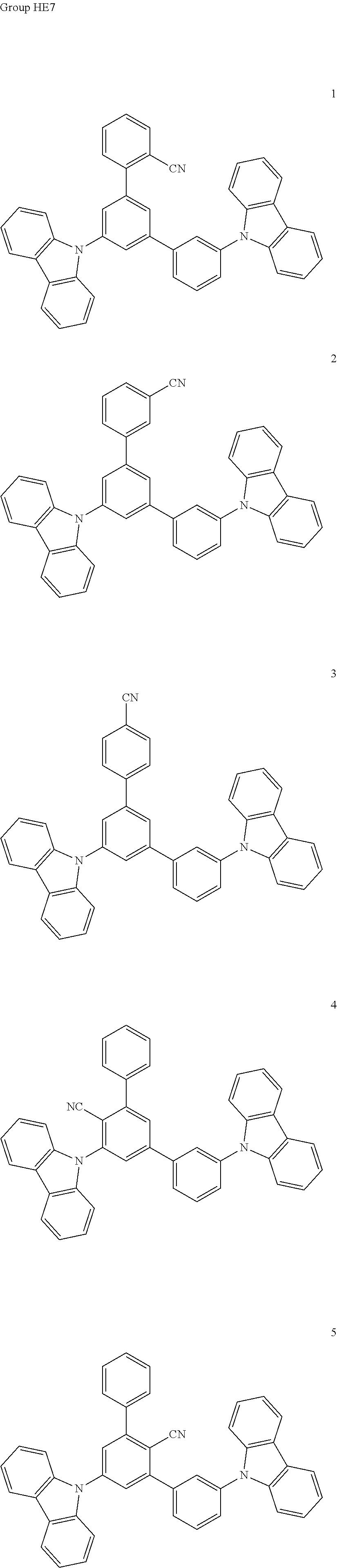 [SMILES: N#Cc1ccccc1-c1cc(-c2cccc(-n3c4ccccc4c4ccccc43)c2)cc(-n2c3ccccc3c3ccccc32)c1 ; N#Cc1cccc(-c2cc(-c3cccc(-n4c5ccccc5c5ccccc54)c3)cc(-n3c4ccccc4c4ccccc43)c2)c1 ; N#Cc1ccc(-c2cc(-c3cccc(-n4c5ccccc5c5ccccc54)c3)cc(-n3c4ccccc4c4ccccc43)c2)cc1 ; N#Cc1c(-c2ccccc2)cc(-c2cccc(-n3c4ccccc4c4ccccc43)c2)cc1-n1c2ccccc2c2ccccc21 ; N#Cc1c(-c2ccccc2)cc(-n2c3ccccc3c3ccccc32)cc1-c1cccc(-n2c3ccccc3c3ccccc32)c1]

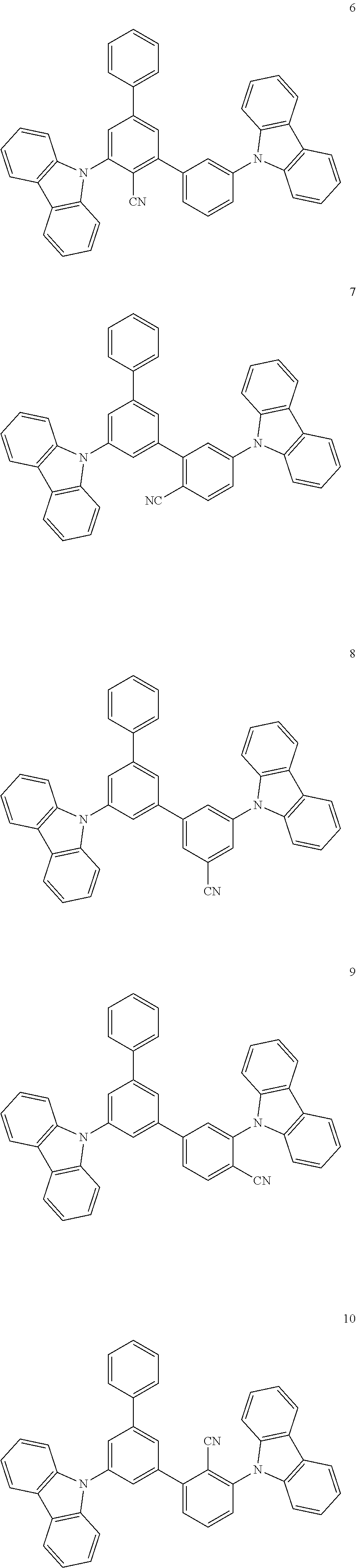 [SMILES: N#Cc1c(-c2cccc(-n3c4ccccc4c4ccccc43)c2)cc(-c2ccccc2)cc1-n1c2ccccc2c2ccccc21 ; N#Cc1ccc(-n2c3ccccc3c3ccccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3ccccc32)c1 ; N#Cc1ccc(-c2cc(-c3ccccc3)cc(-n3c4ccccc4c4ccccc43)c2)cc1-n1c2ccccc2c2ccccc21 ; N#Cc1c(-c2cc(-c3ccccc3)cc(-n3c4ccccc4c4ccccc43)c2)cccc1-n1c2ccccc2c2ccccc21]

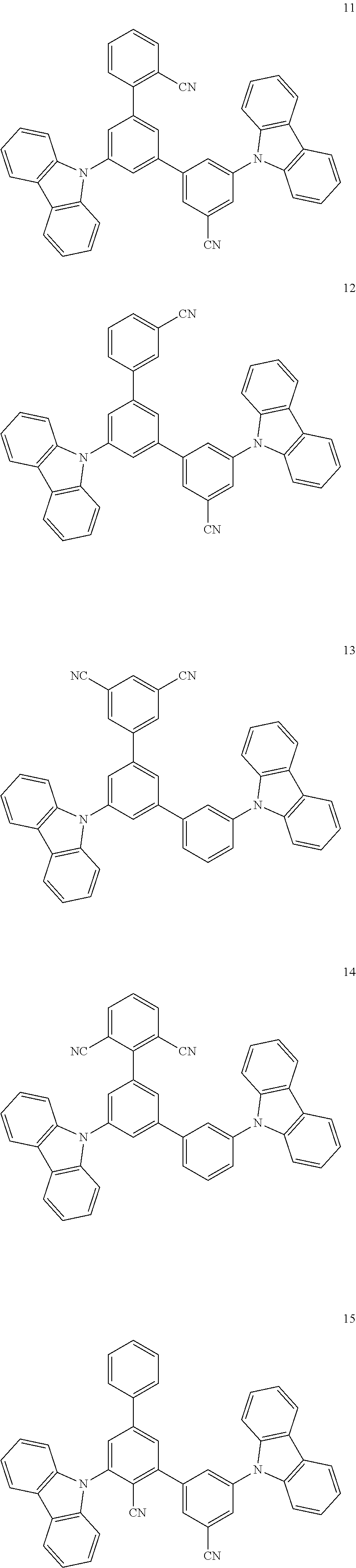 [SMILES: N#Cc1cc(-c2cc(-c3ccccc3C#N)cc(-n3c4ccccc4c4ccccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4ccccc43)c2)c1 ; N#Cc1cc(C#N)cc(-c2cc(-c3cccc(-n4c5ccccc5c5ccccc54)c3)cc(-n3c4ccccc4c4ccccc43)c2)c1 ; N#Cc1cccc(C#N)c1-c1cc(-c2cccc(-n3c4ccccc4c4ccccc43)c2)cc(-n2c3ccccc3c3ccccc32)c1 ; N#Cc1cc(-c2cc(-c3ccccc3)cc(-n3c4ccccc4c4ccccc43)c2C#N)cc(-n2c3ccccc3c3ccccc32)c1]

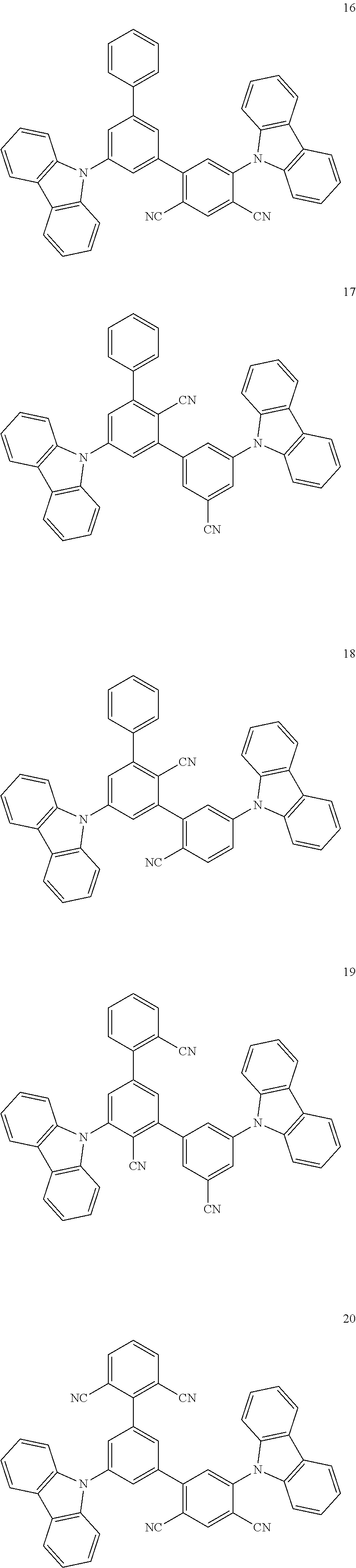 [SMILES: N#Cc1cc(C#N)c(-n2c3ccccc3c3ccccc32)cc1-c1cc(-c2ccccc2)cc(-n2c3ccccc3c3ccccc32)c1 ; N#Cc1cc(-c2cc(-n3c4ccccc4c4ccccc43)cc(-c3ccccc3)c2C#N)cc(-n2c3ccccc3c3ccccc32)c1 ; N#Cc1ccc(-n2c3ccccc3c3ccccc32)cc1-c1cc(-n2c3ccccc3c3ccccc32)cc(-c2ccccc2)c1C#N ; N#Cc1cc(-c2cc(-c3ccccc3C#N)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2c(C#N)cccc2C#N)cc(-n2c3ccccc3c3ccccc32)c1]

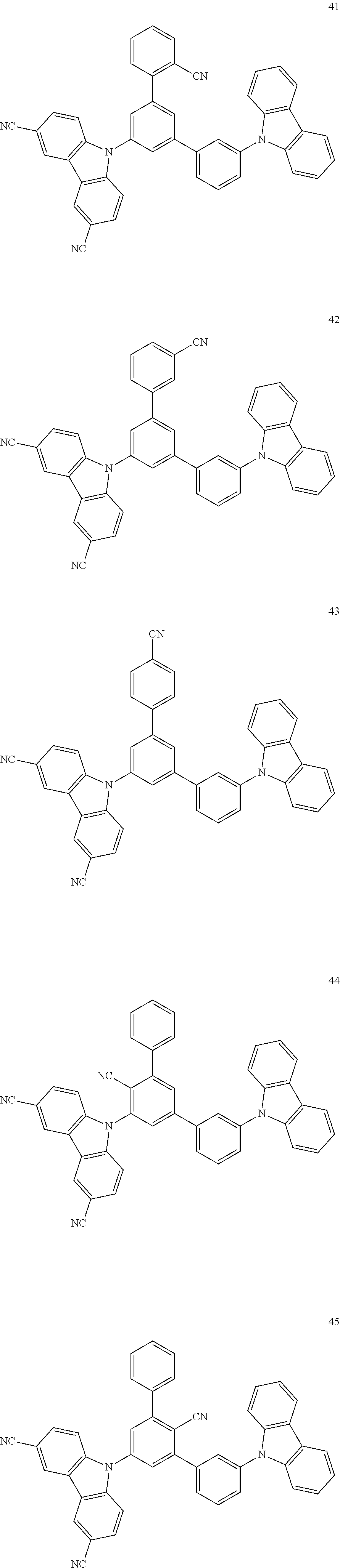 [SMILES: N#Cc1ccc2c(c1)c1cc(C#N)ccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccc(C#N)cc4c4cc(C#N)ccc43)c2)c1 ; N#Cc1ccc(-c2cc(-c3cccc(-n4c5ccccc5c5ccccc54)c3)cc(-n3c4ccc(C#N)cc4c4cc(C#N)ccc43)c2)cc1 ; N#Cc1ccc2c(c1)c1cc(C#N)ccc1n2-c1cc(-c2cccc(-n3c4ccccc4c4ccccc43)c2)cc(-c2ccccc2)c1C#N ; N#Cc1ccc2c(c1)c1cc(C#N)ccc1n2-c1cc(-c2ccccc2)c(C#N)c(-c2cccc(-n3c4ccccc4c4ccccc43)c2)c1]

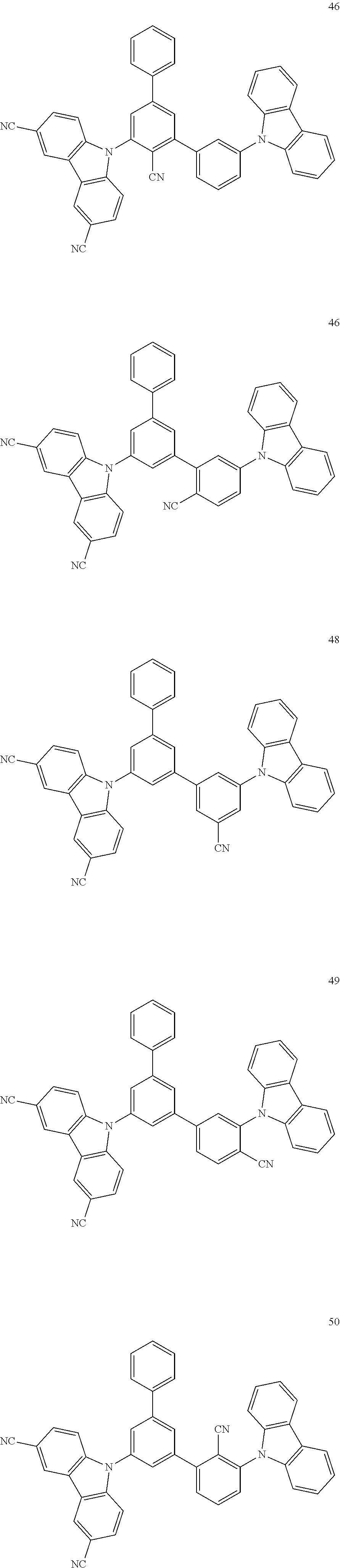 [SMILES: N#Cc1ccc2c(c1)c1cc(C#N)ccc1n2-c1cc(-c2ccccc2)cc(-c2cccc(-n3c4ccccc4c4ccccc43)c2)c1C#N ; N#Cc1ccc2c(c1)c1cc(C#N)ccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccc(C#N)cc4c4cc(C#N)ccc43)c2)cc(-n2c3ccccc3c3ccccc32)c1 ; N#Cc1ccc2c(c1)c1cc(C#N)ccc1n2-c1cc(-c2ccccc2)cc(-c2ccc(C#N)c(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2C#N)c1]

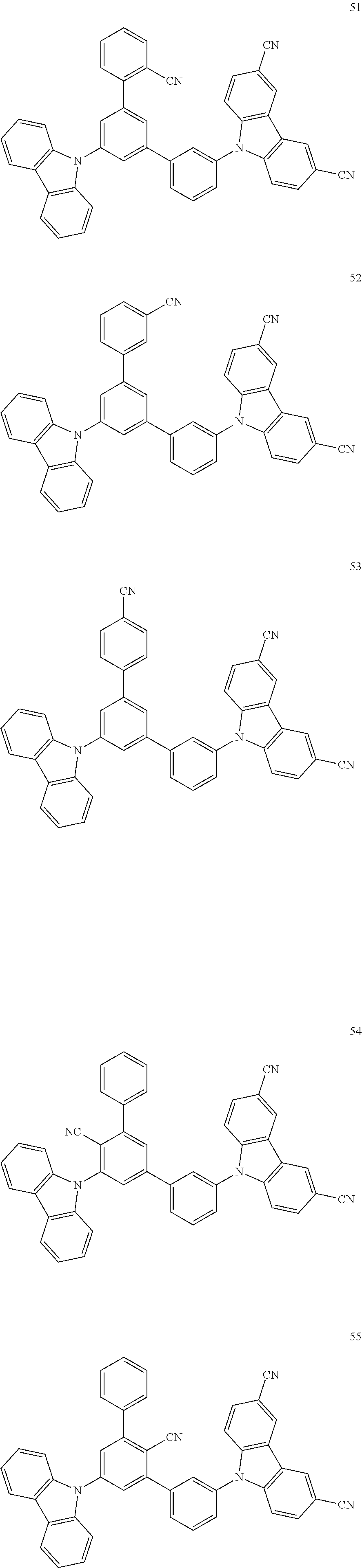 [SMILES: N#Cc1ccc2c(c1)c1cc(C#N)ccc1n2-c1cccc(-c2cc(-c3ccccc3C#N)cc(-n3c4ccccc4c4ccccc43)c2)c1 ; N#Cc1cccc(-c2cc(-c3cccc(-n4c5ccc(C#N)cc5c5cc(C#N)ccc54)c3)cc(-n3c4ccccc4c4ccccc43)c2)c1 ; N#Cc1ccc(-c2cc(-c3cccc(-n4c5ccc(C#N)cc5c5cc(C#N)ccc54)c3)cc(-n3c4ccccc4c4ccccc43)c2)cc1 ; N#Cc1ccc2c(c1)c1cc(C#N)ccc1n2-c1cccc(-c2cc(-c3ccccc3)c(C#N)c(-n3c4ccccc4c4ccccc43)c2)c1 ; N#Cc1ccc2c(c1)c1cc(C#N)ccc1n2-c1cccc(-c2cc(-n3c4ccccc4c4ccccc43)cc(-c3ccccc3)c2C#N)c1]

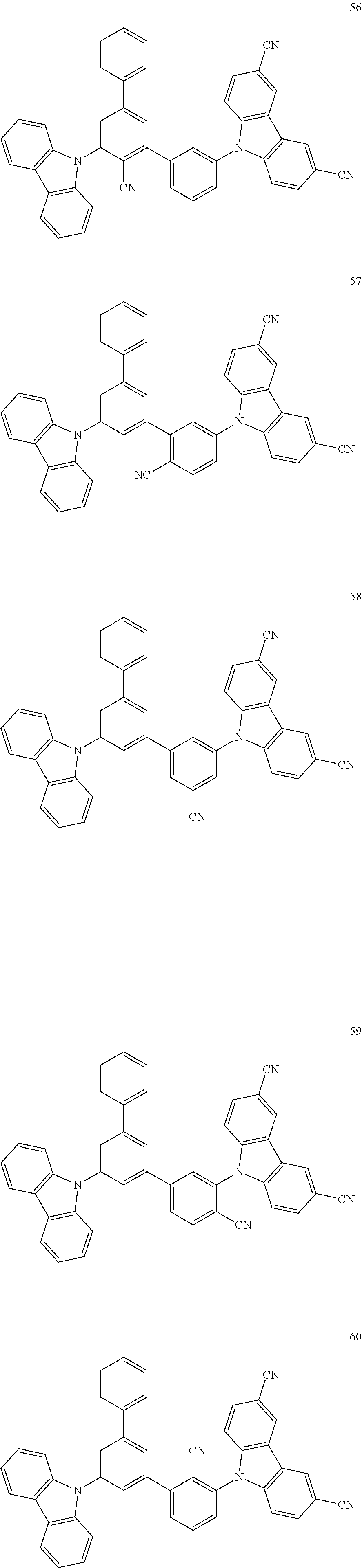 [SMILES: N#Cc1ccc2c(c1)c1cc(C#N)ccc1n2-c1cccc(-c2cc(-c3ccccc3)cc(-n3c4ccccc4c4ccccc43)c2C#N)c1 ; N#Cc1ccc2c(c1)c1cc(C#N)ccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccc(C#N)cc3c3cc(C#N)ccc32)c1 ; N#Cc1ccc2c(c1)c1cc(C#N)ccc1n2-c1cc(-c2cc(-c3ccccc3)cc(-n3c4ccccc4c4ccccc43)c2)ccc1C#N ; N#Cc1ccc2c(c1)c1cc(C#N)ccc1n2-c1cccc(-c2cc(-c3ccccc3)cc(-n3c4ccccc4c4ccccc43)c2)c1C#N]

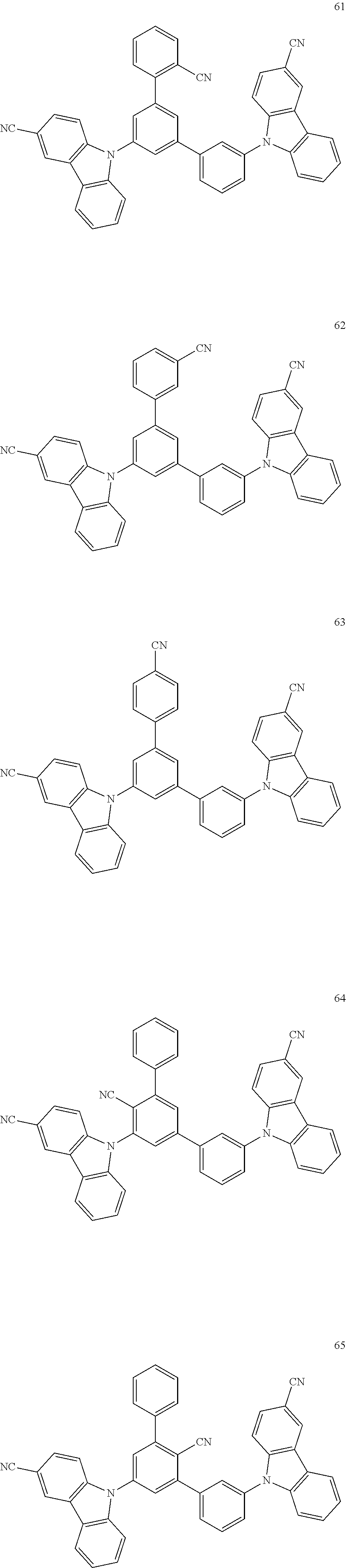 [SMILES: N#Cc1ccc2c(c1)c1ccccc1n2-c1cccc(-c2cc(-c3ccccc3C#N)cc(-n3c4ccccc4c4cc(C#N)ccc43)c2)c1 ; N#Cc1cccc(-c2cc(-c3cccc(-n4c5ccccc5c5cc(C#N)ccc54)c3)cc(-n3c4ccccc4c4cc(C#N)ccc43)c2)c1 ; N#Cc1ccc(-c2cc(-c3cccc(-n4c5ccccc5c5cc(C#N)ccc54)c3)cc(-n3c4ccccc4c4cc(C#N)ccc43)c2)cc1 ; N#Cc1ccc2c(c1)c1ccccc1n2-c1cccc(-c2cc(-c3ccccc3)c(C#N)c(-n3c4ccccc4c4cc(C#N)ccc43)c2)c1 ; N#Cc1ccc2c(c1)c1ccccc1n2-c1cccc(-c2cc(-n3c4ccccc4c4cc(C#N)ccc43)cc(-c3ccccc3)c2C#N)c1]

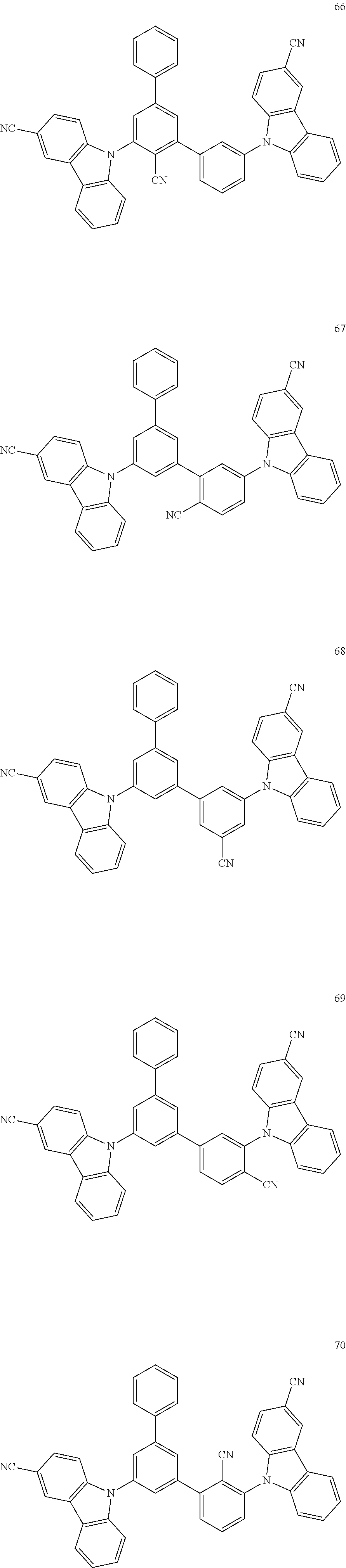 [SMILES: N#Cc1ccc2c(c1)c1ccccc1n2-c1cccc(-c2cc(-c3ccccc3)cc(-n3c4ccccc4c4cc(C#N)ccc43)c2C#N)c1 ; N#Cc1ccc2c(c1)c1ccccc1n2-c1cc(-c2ccccc2)cc(-c2cc(-n3c4ccccc4c4cc(C#N)ccc43)ccc2C#N)c1 ; N#Cc1cc(-c2cc(-c3ccccc3)cc(-n3c4ccccc4c4cc(C#N)ccc43)c2)cc(-n2c3ccccc3c3cc(C#N)ccc32)c1 ; N#Cc1ccc2c(c1)c1ccccc1n2-c1cc(-c2ccccc2)cc(-c2ccc(C#N)c(-n3c4ccccc4c4cc(C#N)ccc43)c2)c1 ; N#Cc1ccc2c(c1)c1ccccc1n2-c1cc(-c2ccccc2)cc(-c2cccc(-n3c4ccccc4c4cc(C#N)ccc43)c2C#N)c1]

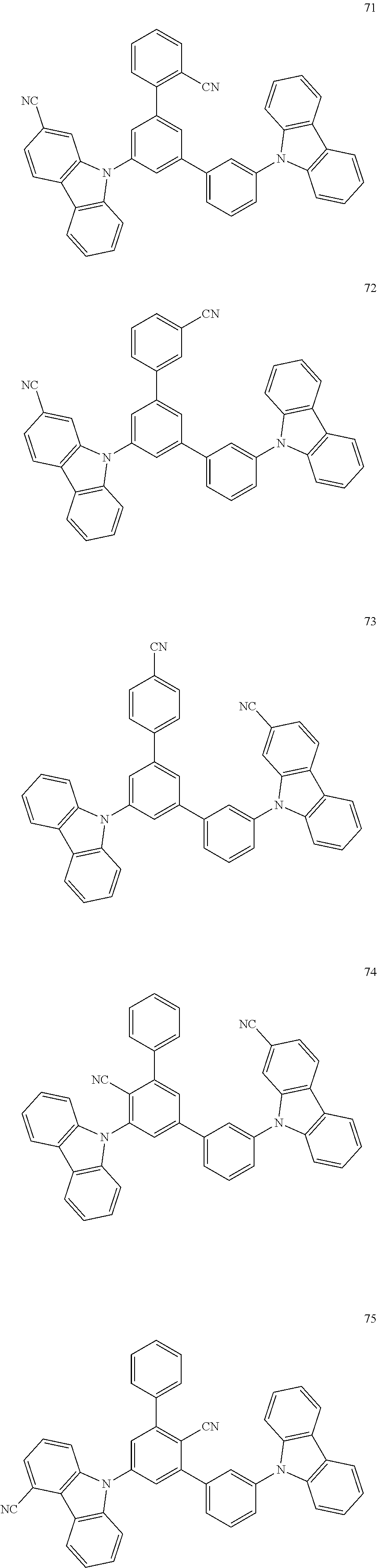 [SMILES: N#Cc1ccc2c3ccccc3n(-c3cc(-c4cccc(-n5c6ccccc6c6ccccc65)c4)cc(-c4ccccc4C#N)c3)c2c1 ; N#Cc1cccc(-c2cc(-c3cccc(-n4c5ccccc5c5ccccc54)c3)cc(-n3c4ccccc4c4ccc(C#N)cc43)c2)c1 ; N#Cc1ccc(-c2cc(-c3cccc(-n4c5ccccc5c5ccc(C#N)cc54)c3)cc(-n3c4ccccc4c4ccccc43)c2)cc1 ; N#Cc1ccc2c3ccccc3n(-c3cccc(-c4cc(-c5ccccc5)c(C#N)c(-n5c6ccccc6c6ccccc65)c4)c3)c2c1 ; N#Cc1c(-c2ccccc2)cc(-n2c3ccccc3c3c(C#N)cccc32)cc1-c1cccc(-n2c3ccccc3c3ccccc32)c1]

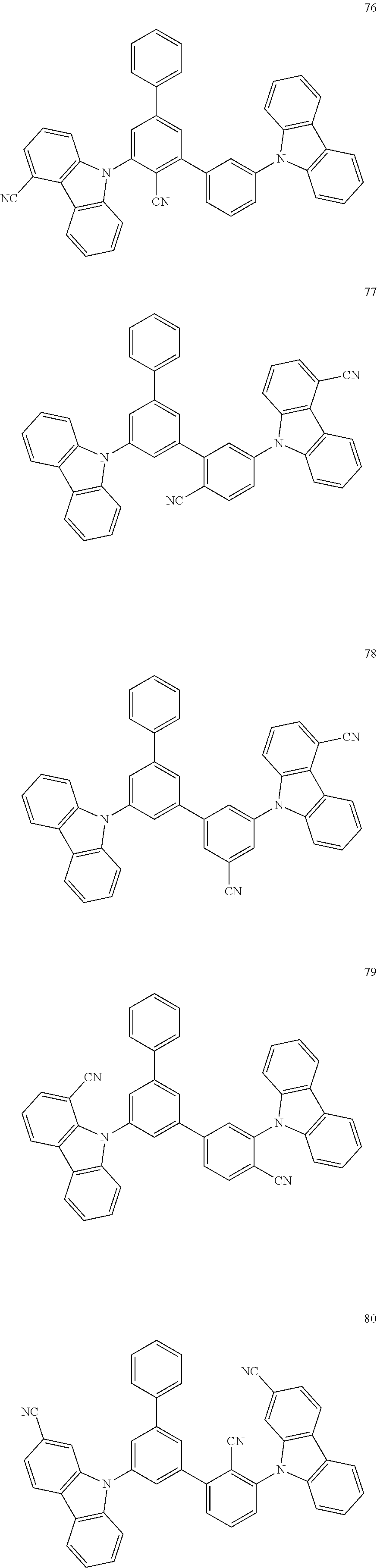 [SMILES: N#Cc1c(-c2cccc(-n3c4ccccc4c4ccccc43)c2)cc(-c2ccccc2)cc1-n1c2ccccc2c2c(C#N)cccc21 ; N#Cc1ccc(-n2c3ccccc3c3c(C#N)cccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3c(C#N)cccc32)c1 ; N#Cc1ccc(-c2cc(-c3ccccc3)cc(-n3c4ccccc4c4cccc(C#N)c43)c2)cc1-n1c2ccccc2c2ccccc21 ; N#Cc1ccc2c3ccccc3n(-c3cc(-c4ccccc4)cc(-c4cccc(-n5c6ccccc6c6ccc(C#N)cc65)c4C#N)c3)c2c1]

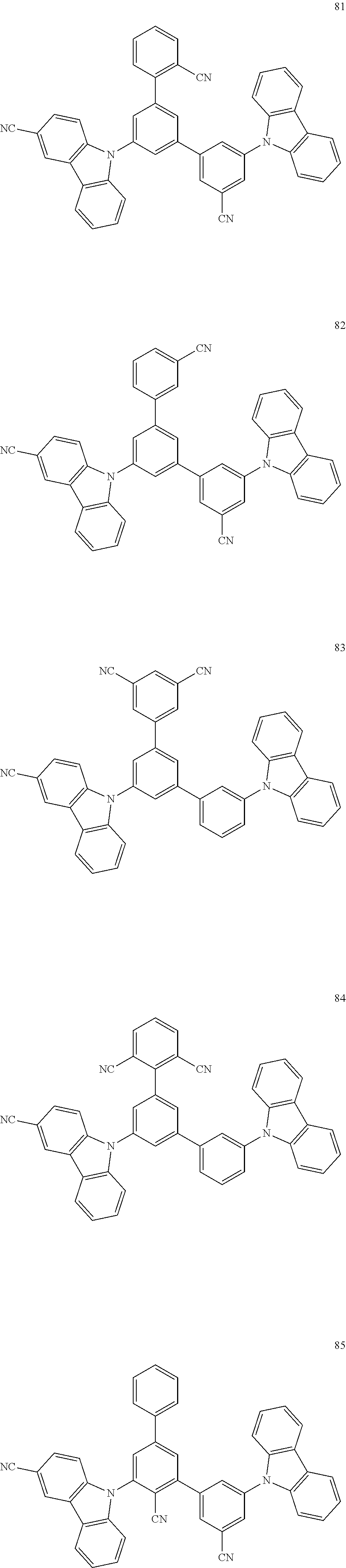 [SMILES: N#Cc1cc(-c2cc(-c3ccccc3C#N)cc(-n3c4ccccc4c4cc(C#N)ccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4cc(C#N)ccc43)c2)c1 ; N#Cc1cc(C#N)cc(-c2cc(-c3cccc(-n4c5ccccc5c5ccccc54)c3)cc(-n3c4ccccc4c4cc(C#N)ccc43)c2)c1 ; N#Cc1ccc2c(c1)c1ccccc1n2-c1cc(-c2cccc(-n3c4ccccc4c4ccccc43)c2)cc(-c2c(C#N)cccc2C#N)c1 ; N#Cc1cc(-c2cc(-c3ccccc3)cc(-n3c4ccccc4c4cc(C#N)ccc43)c2C#N)cc(-n2c3ccccc3c3ccccc32)c1]

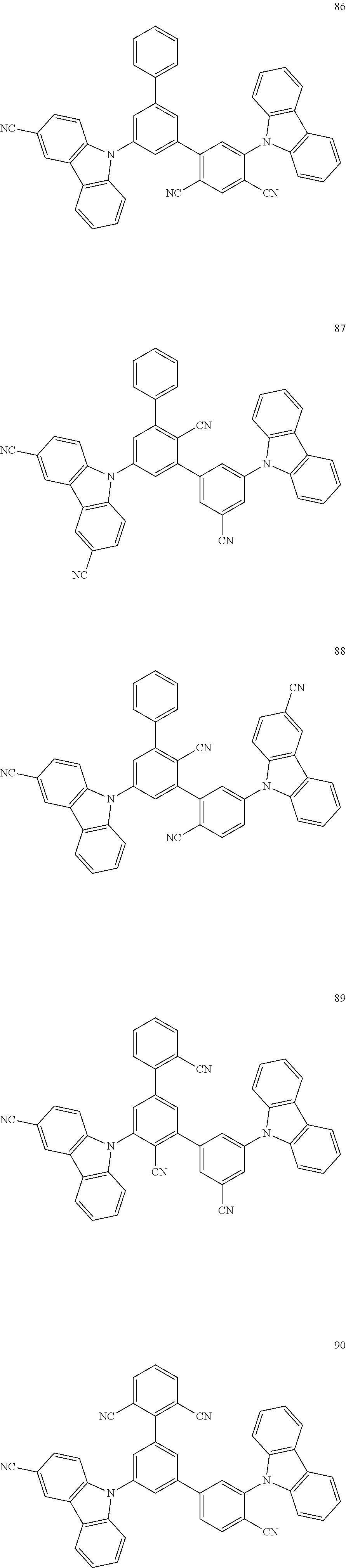 [SMILES: N#Cc1ccc2c(c1)c1ccccc1n2-c1cc(-c2ccccc2)cc(-c2cc(-n3c4ccccc4c4ccccc43)c(C#N)cc2C#N)c1 ; N#Cc1cc(-c2cc(-n3c4ccc(C#N)cc4c4cc(C#N)ccc43)cc(-c3ccccc3)c2C#N)cc(-n2c3ccccc3c3ccccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccc(C#N)c(-n3c4ccccc4c4ccccc43)c2)cc(-c2c(C#N)cccc2C#N)c1]

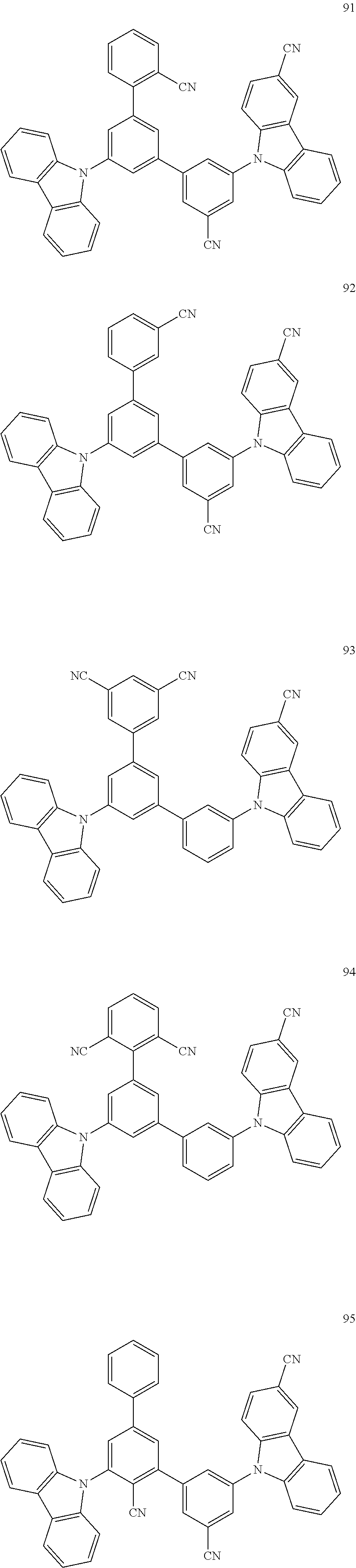 [SMILES: N#Cc1cc(-c2cc(-c3ccccc3C#N)cc(-n3c4ccccc4c4ccccc43)c2)cc(-n2c3ccccc3c3cc(C#N)ccc32)c1 ; N#Cc1cccc(-c2cc(-c3cc(C#N)cc(-n4c5ccccc5c5cc(C#N)ccc54)c3)cc(-n3c4ccccc4c4ccccc43)c2)c1 ; N#Cc1cc(C#N)cc(-c2cc(-c3cccc(-n4c5ccccc5c5cc(C#N)ccc54)c3)cc(-n3c4ccccc4c4ccccc43)c2)c1 ; N#Cc1ccc2c(c1)c1ccccc1n2-c1cccc(-c2cc(-c3c(C#N)cccc3C#N)cc(-n3c4ccccc4c4ccccc43)c2)c1 ; N#Cc1cc(-c2cc(-c3ccccc3)cc(-n3c4ccccc4c4ccccc43)c2C#N)cc(-n2c3ccccc3c3cc(C#N)ccc32)c1]

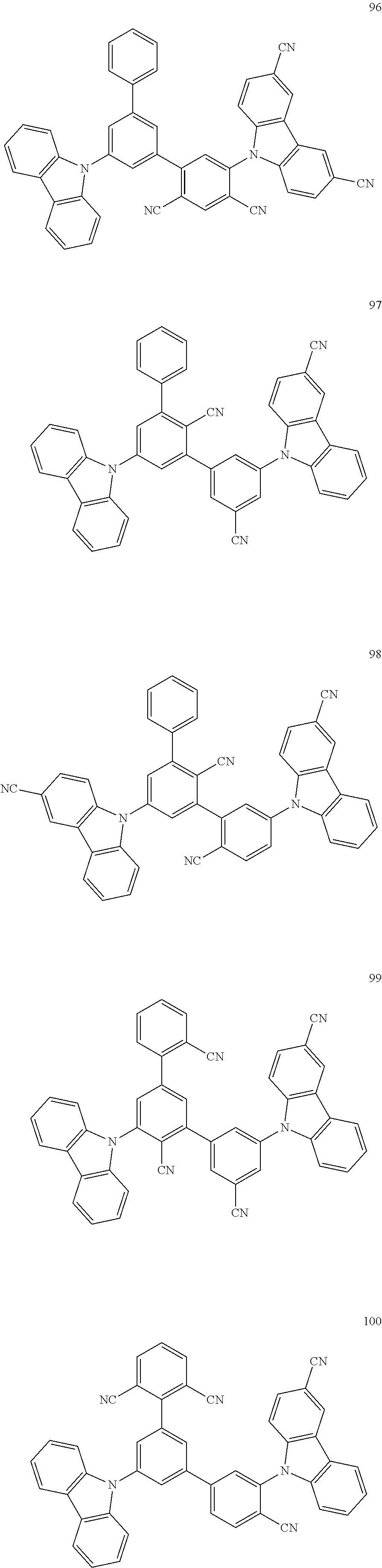 [SMILES: N#Cc1ccc2c(c1)c1cc(C#N)ccc1n2-c1cc(-c2cc(-c3ccccc3)cc(-n3c4ccccc4c4ccccc43)c2)c(C#N)cc1C#N ; N#Cc1cc(-c2cc(-n3c4ccccc4c4ccccc43)cc(-c3ccccc3)c2C#N)cc(-n2c3ccccc3c3cc(C#N)ccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4ccccc43)c2C#N)cc(-n2c3ccccc3c3cc(C#N)ccc32)c1 ; N#Cc1ccc2c(c1)c1ccccc1n2-c1cc(-c2cc(-c3c(C#N)cccc3C#N)cc(-n3c4ccccc4c4ccccc43)c2)ccc1C#N]

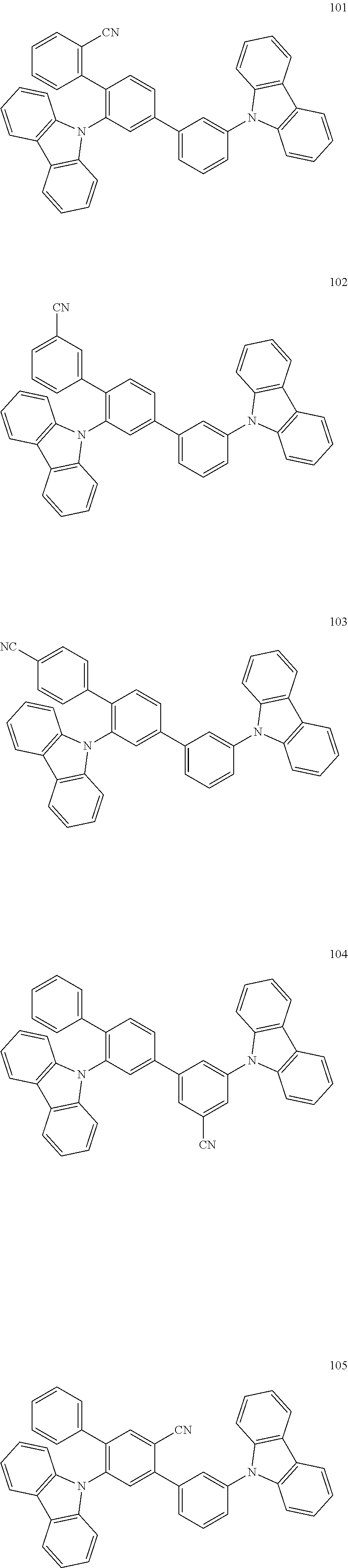 [SMILES: N#Cc1ccccc1-c1ccc(-c2cccc(-n3c4ccccc4c4ccccc43)c2)cc1-n1c2ccccc2c2ccccc21 ; N#Cc1cccc(-c2ccc(-c3cccc(-n4c5ccccc5c5ccccc54)c3)cc2-n2c3ccccc3c3ccccc32)c1 ; N#Cc1ccc(-c2ccc(-c3cccc(-n4c5ccccc5c5ccccc54)c3)cc2-n2c3ccccc3c3ccccc32)cc1 ; N#Cc1cc(-c2ccc(-c3ccccc3)c(-n3c4ccccc4c4ccccc43)c2)cc(-n2c3ccccc3c3ccccc32)c1 ; N#Cc1cc(-c2ccccc2)c(-n2c3ccccc3c3ccccc32)cc1-c1cccc(-n2c3ccccc3c3ccccc32)c1]

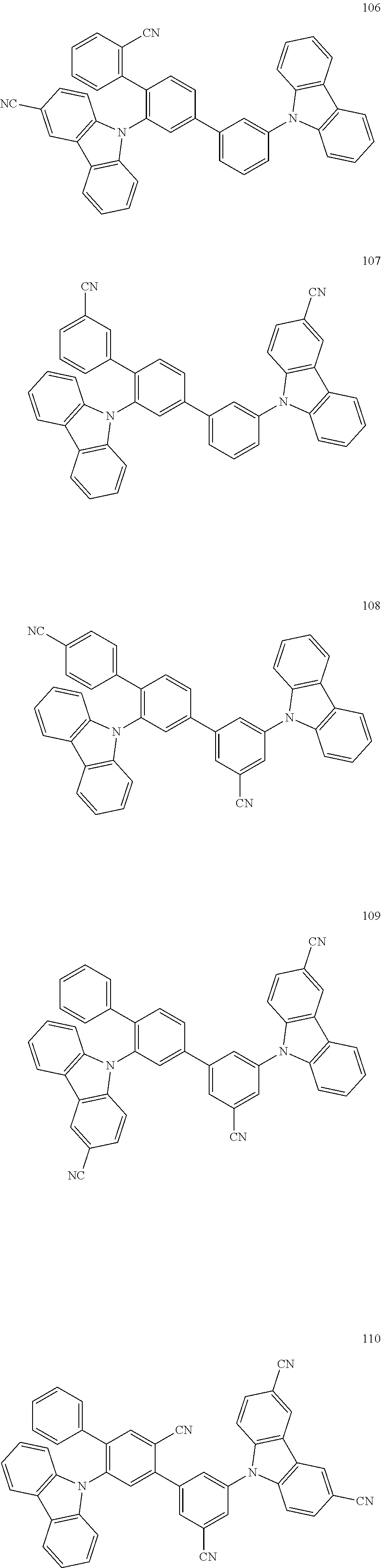 [SMILES: N#Cc1ccc2c(c1)c1ccccc1n2-c1cc(-c2cccc(-n3c4ccccc4c4ccccc43)c2)ccc1-c1ccccc1C#N ; N#Cc1cccc(-c2ccc(-c3cccc(-n4c5ccccc5c5cc(C#N)ccc54)c3)cc2-n2c3ccccc3c3ccccc32)c1 ; N#Cc1ccc(-c2ccc(-c3cc(C#N)cc(-n4c5ccccc5c5ccccc54)c3)cc2-n2c3ccccc3c3ccccc32)cc1 ; N#Cc1cc(-c2ccc(-c3ccccc3)c(-n3c4ccccc4c4cc(C#N)ccc43)c2)cc(-n2c3ccccc3c3cc(C#N)ccc32)c1 ; N#Cc1cc(-c2cc(-n3c4ccccc4c4ccccc43)c(-c3ccccc3)cc2C#N)cc(-n2c3ccc(C#N)cc3c3cc(C#N)ccc32)c1]

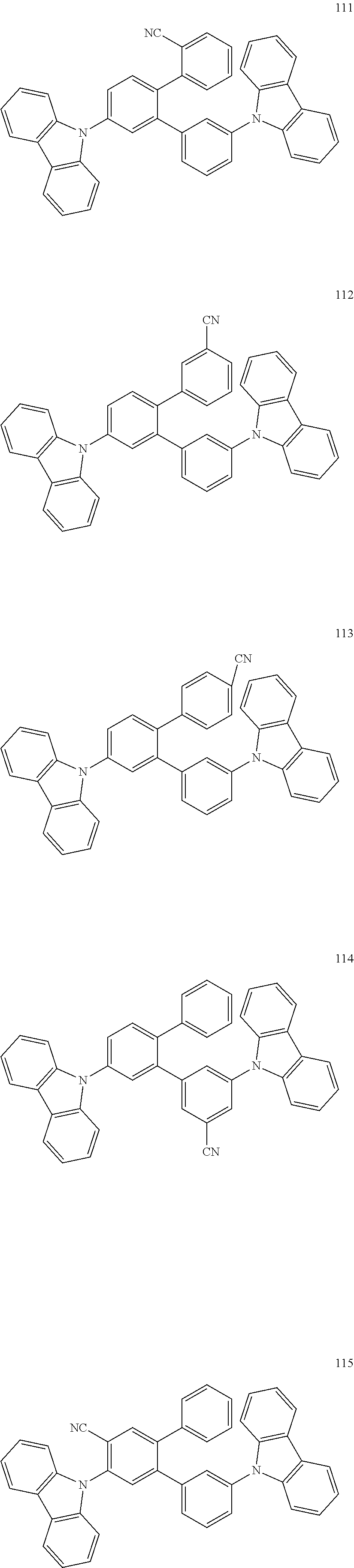 [SMILES: N#Cc1ccccc1-c1ccc(-n2c3ccccc3c3ccccc32)cc1-c1cccc(-n2c3ccccc3c3ccccc32)c1 ; N#Cc1cccc(-c2ccc(-n3c4ccccc4c4ccccc43)cc2-c2cccc(-n3c4ccccc4c4ccccc43)c2)c1 ; N#Cc1ccc(-c2ccc(-n3c4ccccc4c4ccccc43)cc2-c2cccc(-n3c4ccccc4c4ccccc43)c2)cc1 ; N#Cc1cc(-c2cc(-n3c4ccccc4c4ccccc43)ccc2-c2ccccc2)cc(-n2c3ccccc3c3ccccc32)c1 ; N#Cc1cc(-c2ccccc2)c(-c2cccc(-n3c4ccccc4c4ccccc43)c2)cc1-n1c2ccccc2c2ccccc21]

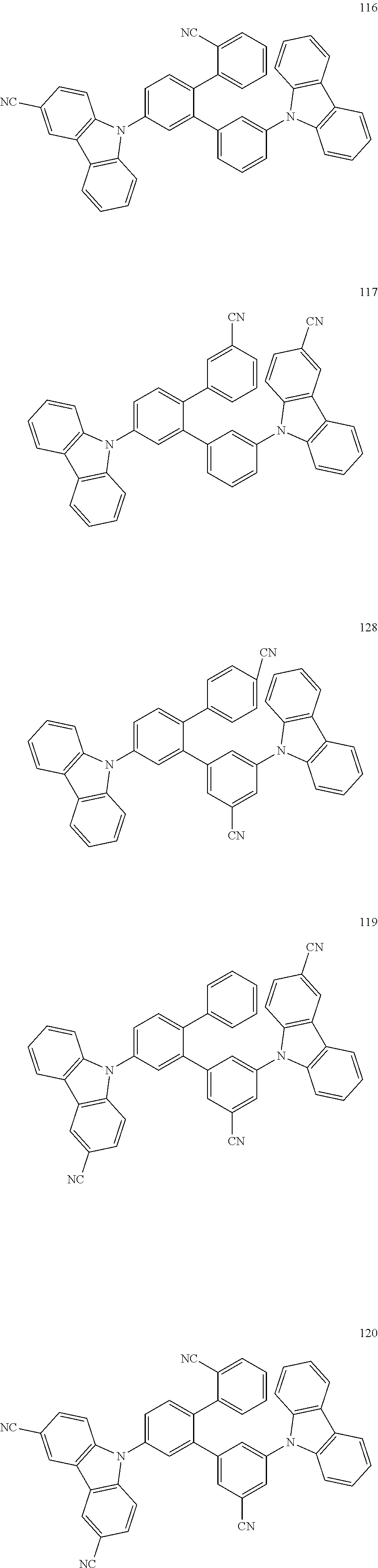 [SMILES: N#Cc1ccc2c(c1)c1ccccc1n2-c1ccc(-c2ccccc2C#N)c(-c2cccc(-n3c4ccccc4c4ccccc43)c2)c1 ; N#Cc1cccc(-c2ccc(-n3c4ccccc4c4ccccc43)cc2-c2cccc(-n3c4ccccc4c4cc(C#N)ccc43)c2)c1 ; N#Cc1ccc(-c2ccc(-n3c4ccccc4c4ccccc43)cc2-c2cc(C#N)cc(-n3c4ccccc4c4ccccc43)c2)cc1 ; N#Cc1cc(-c2cc(-n3c4ccccc4c4cc(C#N)ccc43)ccc2-c2ccccc2)cc(-n2c3ccccc3c3cc(C#N)ccc32)c1 ; N#Cc1cc(-c2cc(-n3c4ccc(C#N)cc4c4cc(C#N)ccc43)ccc2-c2ccccc2C#N)cc(-n2c3ccccc3c3ccccc32)c1]

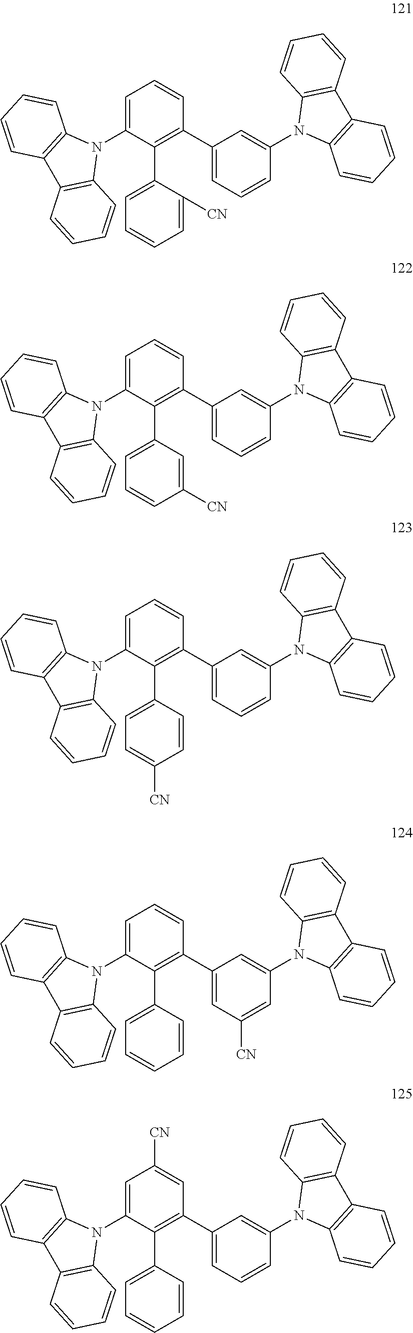 [SMILES: N#Cc1ccccc1-c1c(-c2cccc(-n3c4ccccc4c4ccccc43)c2)cccc1-n1c2ccccc2c2ccccc21 ; N#Cc1cccc(-c2c(-c3cccc(-n4c5ccccc5c5ccccc54)c3)cccc2-n2c3ccccc3c3ccccc32)c1 ; N#Cc1ccc(-c2c(-c3cccc(-n4c5ccccc5c5ccccc54)c3)cccc2-n2c3ccccc3c3ccccc32)cc1 ; N#Cc1cc(-c2cccc(-n3c4ccccc4c4ccccc43)c2-c2ccccc2)cc(-n2c3ccccc3c3ccccc32)c1 ; N#Cc1cc(-c2cccc(-n3c4ccccc4c4ccccc43)c2)c(-c2ccccc2)c(-n2c3ccccc3c3ccccc32)c1]

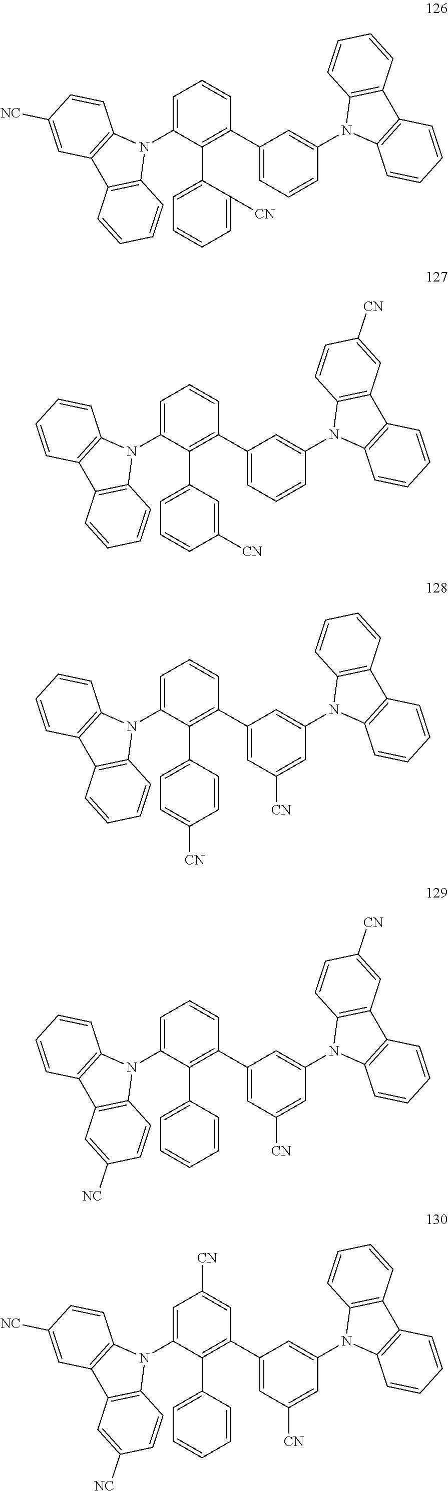 [SMILES: N#Cc1ccc2c(c1)c1ccccc1n2-c1cccc(-c2cccc(-n3c4ccccc4c4ccccc43)c2)c1-c1ccccc1C#N ; N#Cc1cccc(-c2c(-c3cccc(-n4c5ccccc5c5cc(C#N)ccc54)c3)cccc2-n2c3ccccc3c3ccccc32)c1 ; N#Cc1ccc(-c2c(-c3cc(C#N)cc(-n4c5ccccc5c5ccccc54)c3)cccc2-n2c3ccccc3c3ccccc32)cc1 ; N#Cc1cc(-c2cccc(-n3c4ccccc4c4cc(C#N)ccc43)c2-c2ccccc2)cc(-n2c3ccccc3c3cc(C#N)ccc32)c1 ; N#Cc1cc(-c2cc(C#N)cc(-n3c4ccc(C#N)cc4c4cc(C#N)ccc43)c2-c2ccccc2)cc(-n2c3ccccc3c3ccccc32)c1]

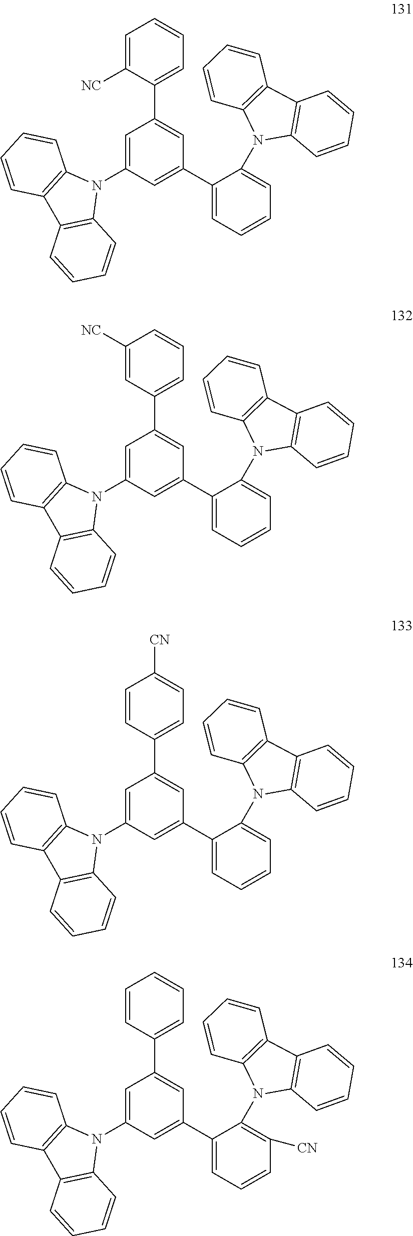 [SMILES: N#Cc1ccccc1-c1cc(-c2ccccc2-n2c3ccccc3c3ccccc32)cc(-n2c3ccccc3c3ccccc32)c1 ; N#Cc1cccc(-c2cc(-c3ccccc3-n3c4ccccc4c4ccccc43)cc(-n3c4ccccc4c4ccccc43)c2)c1 ; N#Cc1ccc(-c2cc(-c3ccccc3-n3c4ccccc4c4ccccc43)cc(-n3c4ccccc4c4ccccc43)c2)cc1 ; N#Cc1cccc(-c2cc(-c3ccccc3)cc(-n3c4ccccc4c4ccccc43)c2)c1-n1c2ccccc2c2ccccc21]

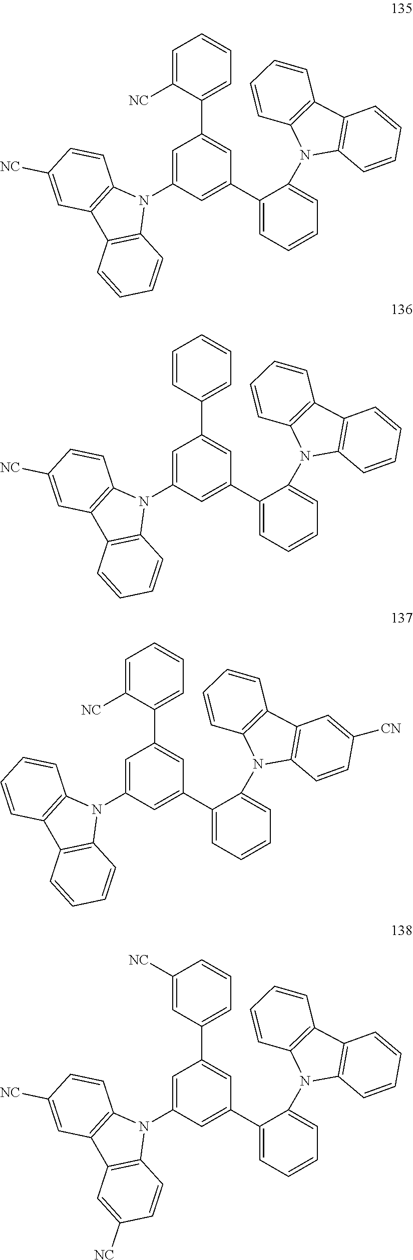 [SMILES: N#Cc1ccc2c(c1)c1ccccc1n2-c1cc(-c2ccccc2C#N)cc(-c2ccccc2-n2c3ccccc3c3ccccc32)c1 ; N#Cc1ccc2c(c1)c1ccccc1n2-c1cc(-c2ccccc2)cc(-c2ccccc2-n2c3ccccc3c3ccccc32)c1 ; N#Cc1ccc2c(c1)c1ccccc1n2-c1ccccc1-c1cc(-c2ccccc2C#N)cc(-n2c3ccccc3c3ccccc32)c1 ; N#Cc1cccc(-c2cc(-c3ccccc3-n3c4ccccc4c4ccccc43)cc(-n3c4ccc(C#N)cc4c4cc(C#N)ccc43)c2)c1]

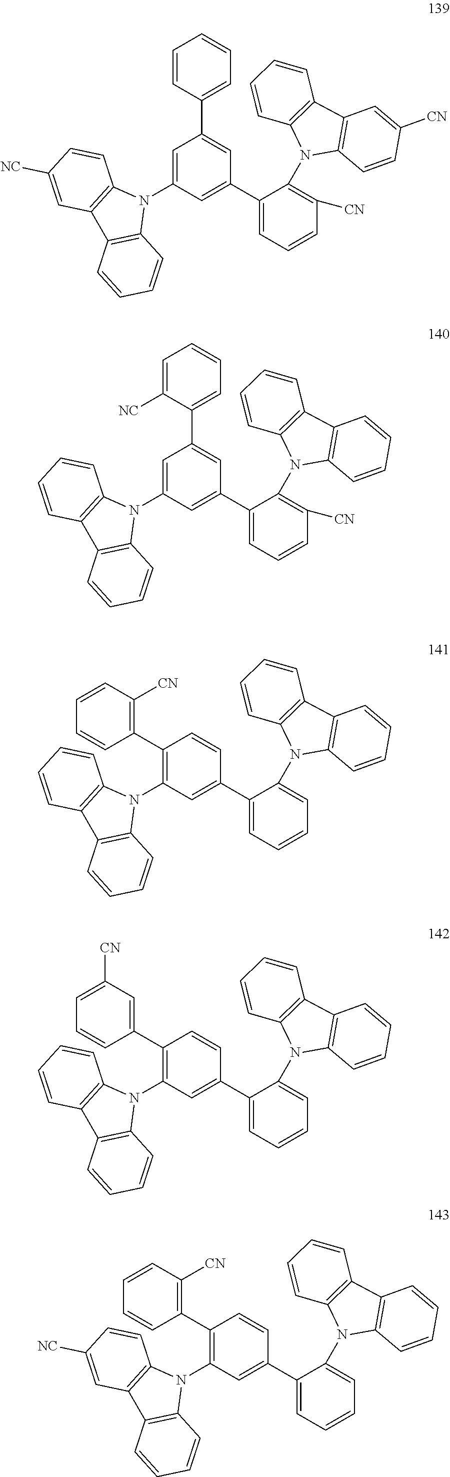 [SMILES: N#Cc1ccc2c(c1)c1ccccc1n2-c1cc(-c2ccccc2)cc(-c2cccc(C#N)c2-n2c3ccccc3c3cc(C#N)ccc32)c1 ; N#Cc1ccccc1-c1cc(-c2cccc(C#N)c2-n2c3ccccc3c3ccccc32)cc(-n2c3ccccc3c3ccccc32)c1 ; N#Cc1ccccc1-c1ccc(-c2ccccc2-n2c3ccccc3c3ccccc32)cc1-n1c2ccccc2c2ccccc21 ; N#Cc1cccc(-c2ccc(-c3ccccc3-n3c4ccccc4c4ccccc43)cc2-n2c3ccccc3c3ccccc32)c1 ; N#Cc1ccc2c(c1)c1ccccc1n2-c1cc(-c2ccccc2-n2c3ccccc3c3ccccc32)ccc1-c1ccccc1C#N]

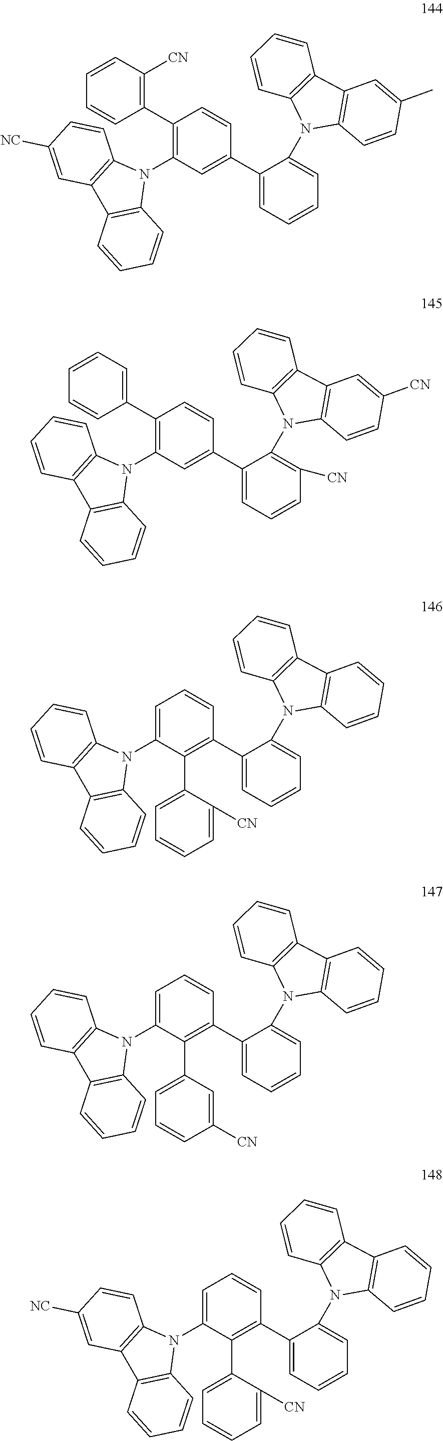 [SMILES: Cc1ccc2c(c1)c1ccccc1n2-c1ccccc1-c1ccc(-c2ccccc2C#N)c(-n2c3ccccc3c3cc(C#N)ccc32)c1 ; N#Cc1ccc2c(c1)c1ccccc1n2-c1c(C#N)cccc1-c1ccc(-c2ccccc2)c(-n2c3ccccc3c3ccccc32)c1 ; N#Cc1ccccc1-c1c(-c2ccccc2-n2c3ccccc3c3ccccc32)cccc1-n1c2ccccc2c2ccccc21 ; N#Cc1cccc(-c2c(-c3ccccc3-n3c4ccccc4c4ccccc43)cccc2-n2c3ccccc3c3ccccc32)c1 ; N#Cc1ccc2c(c1)c1ccccc1n2-c1cccc(-c2ccccc2-n2c3ccccc3c3ccccc32)c1-c1ccccc1C#N]

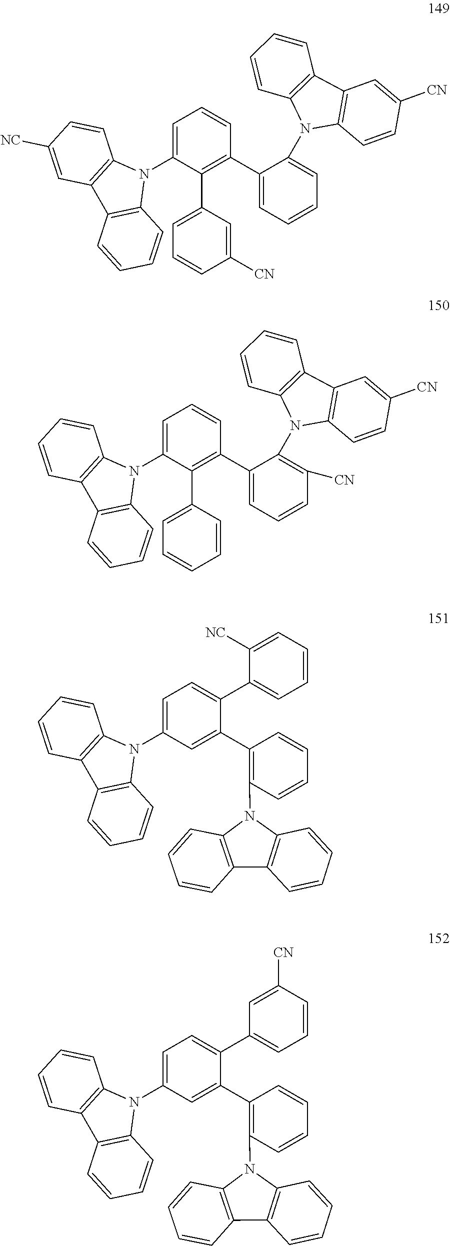 [SMILES: N#Cc1cccc(-c2c(-c3ccccc3-n3c4ccccc4c4cc(C#N)ccc43)cccc2-n2c3ccccc3c3cc(C#N)ccc32)c1 ; N#Cc1ccc2c(c1)c1ccccc1n2-c1c(C#N)cccc1-c1cccc(-n2c3ccccc3c3ccccc32)c1-c1ccccc1 ; N#Cc1ccccc1-c1ccc(-n2c3ccccc3c3ccccc32)cc1-c1ccccc1-n1c2ccccc2c2ccccc21 ; N#Cc1cccc(-c2ccc(-n3c4ccccc4c4ccccc43)cc2-c2ccccc2-n2c3ccccc3c3ccccc32)c1]

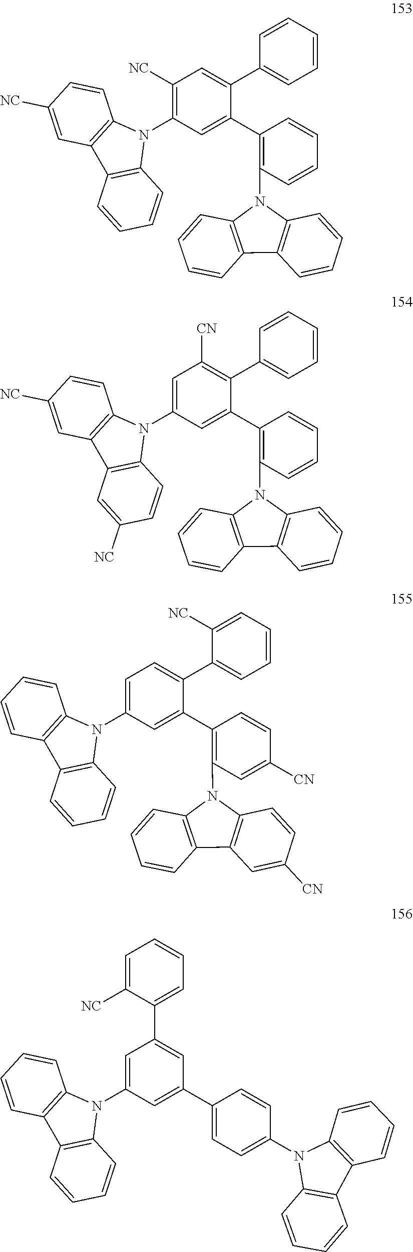

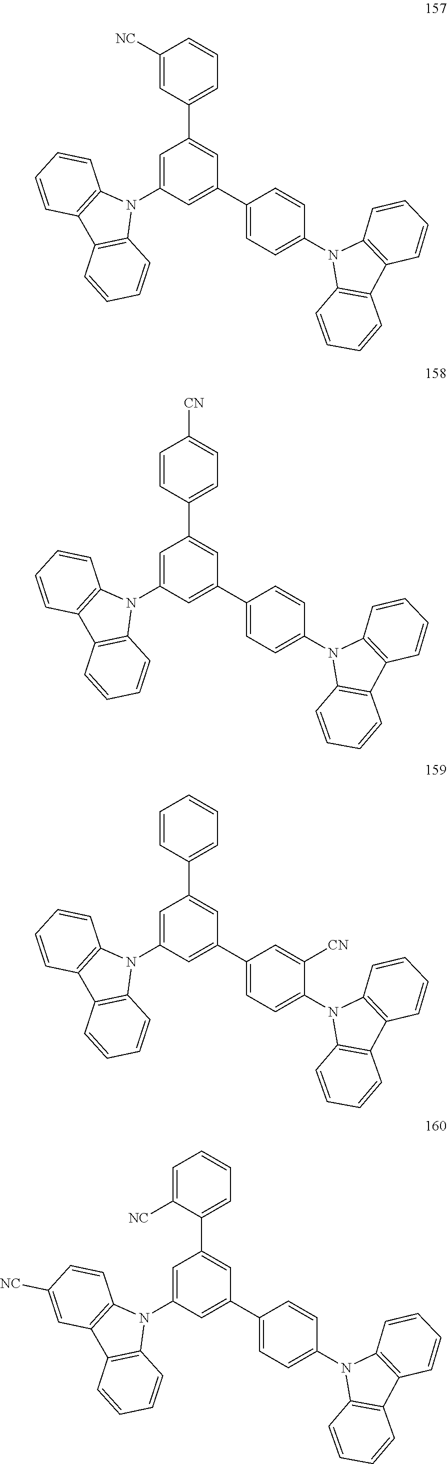 [SMILES: N#Cc1cccc(-c2cc(-c3ccc(-n4c5ccccc5c5ccccc54)cc3)cc(-n3c4ccccc4c4ccccc43)c2)c1 ; N#Cc1ccc(-c2cc(-c3ccc(-n4c5ccccc5c5ccccc54)cc3)cc(-n3c4ccccc4c4ccccc43)c2)cc1 ; N#Cc1cc(-c2cc(-c3ccccc3)cc(-n3c4ccccc4c4ccccc43)c2)ccc1-n1c2ccccc2c2ccccc21 ; N#Cc1ccc2c(c1)c1ccccc1n2-c1cc(-c2ccc(-n3c4ccccc4c4ccccc43)cc2)cc(-c2ccccc2C#N)c1]

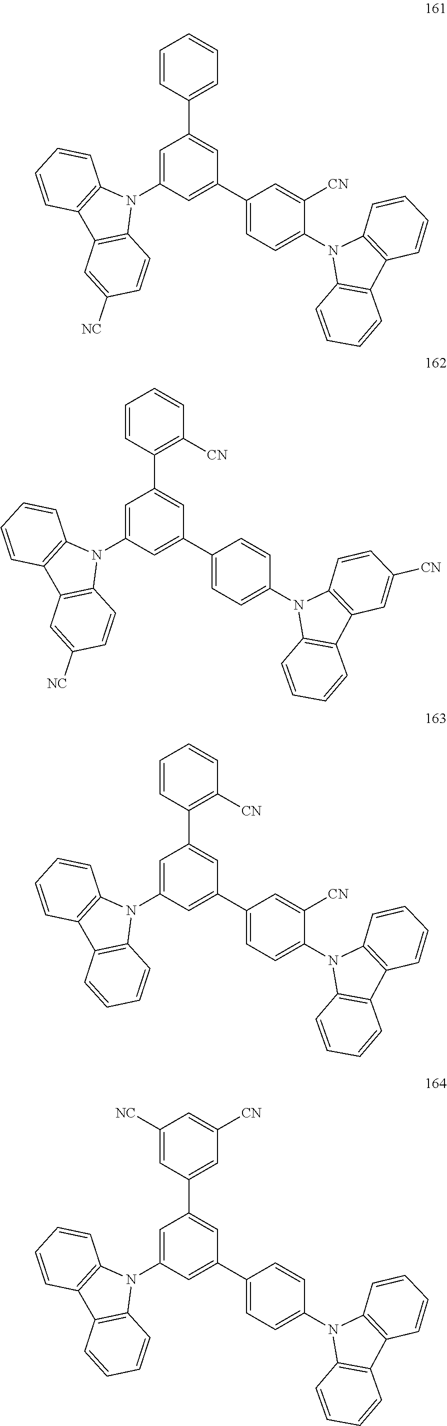 [SMILES: N#Cc1ccc2c(c1)c1ccccc1n2-c1cc(-c2ccccc2)cc(-c2ccc(-n3c4ccccc4c4ccccc43)c(C#N)c2)c1 ; N#Cc1ccc2c(c1)c1ccccc1n2-c1ccc(-c2cc(-c3ccccc3C#N)cc(-n3c4ccccc4c4cc(C#N)ccc43)c2)cc1 ; N#Cc1ccccc1-c1cc(-c2ccc(-n3c4ccccc4c4ccccc43)c(C#N)c2)cc(-n2c3ccccc3c3ccccc32)c1 ; N#Cc1cc(C#N)cc(-c2cc(-c3ccc(-n4c5ccccc5c5ccccc54)cc3)cc(-n3c4ccccc4c4ccccc43)c2)c1]

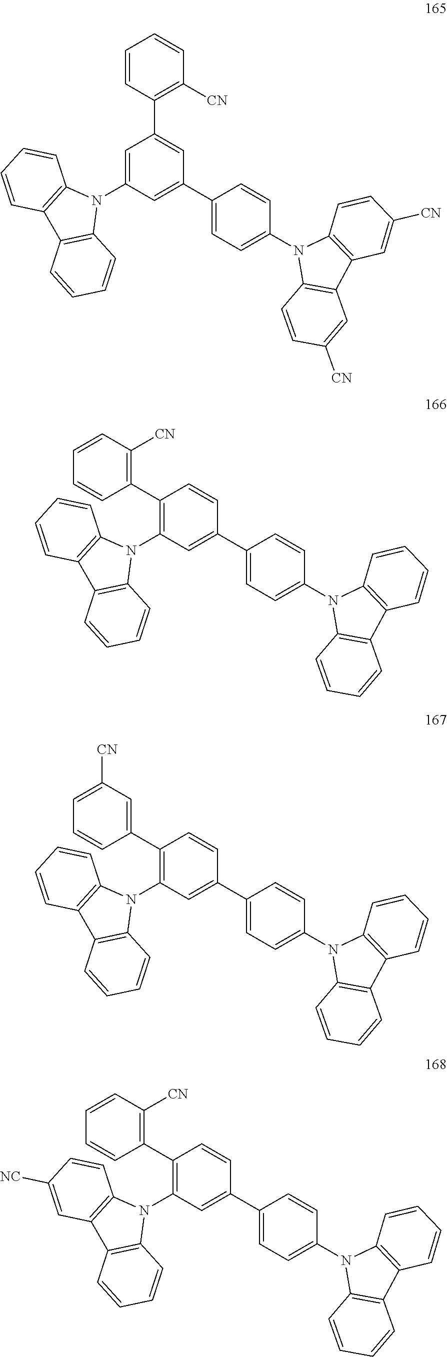 [SMILES: N#Cc1ccc2c(c1)c1cc(C#N)ccc1n2-c1ccc(-c2cc(-c3ccccc3C#N)cc(-n3c4ccccc4c4ccccc43)c2)cc1 ; N#Cc1ccccc1-c1ccc(-c2ccc(-n3c4ccccc4c4ccccc43)cc2)cc1-n1c2ccccc2c2ccccc21 ; N#Cc1cccc(-c2ccc(-c3ccc(-n4c5ccccc5c5ccccc54)cc3)cc2-n2c3ccccc3c3ccccc32)c1 ; N#Cc1ccc2c(c1)c1ccccc1n2-c1cc(-c2ccc(-n3c4ccccc4c4ccccc43)cc2)ccc1-c1ccccc1C#N]

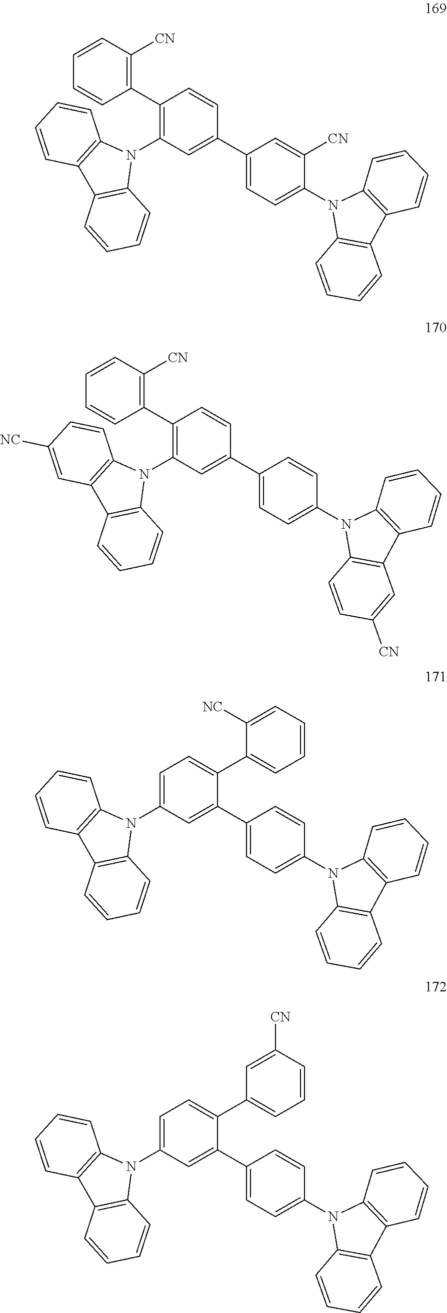 [SMILES: N#Cc1ccccc1-c1ccc(-c2ccc(-n3c4ccccc4c4ccccc43)c(C#N)c2)cc1-n1c2ccccc2c2ccccc21 ; N#Cc1ccc2c(c1)c1ccccc1n2-c1ccc(-c2ccc(-c3ccccc3C#N)c(-n3c4ccccc4c4cc(C#N)ccc43)c2)cc1 ; N#Cc1ccccc1-c1ccc(-n2c3ccccc3c3ccccc32)cc1-c1ccc(-n2c3ccccc3c3ccccc32)cc1 ; N#Cc1cccc(-c2ccc(-n3c4ccccc4c4ccccc43)cc2-c2ccc(-n3c4ccccc4c4ccccc43)cc2)c1]

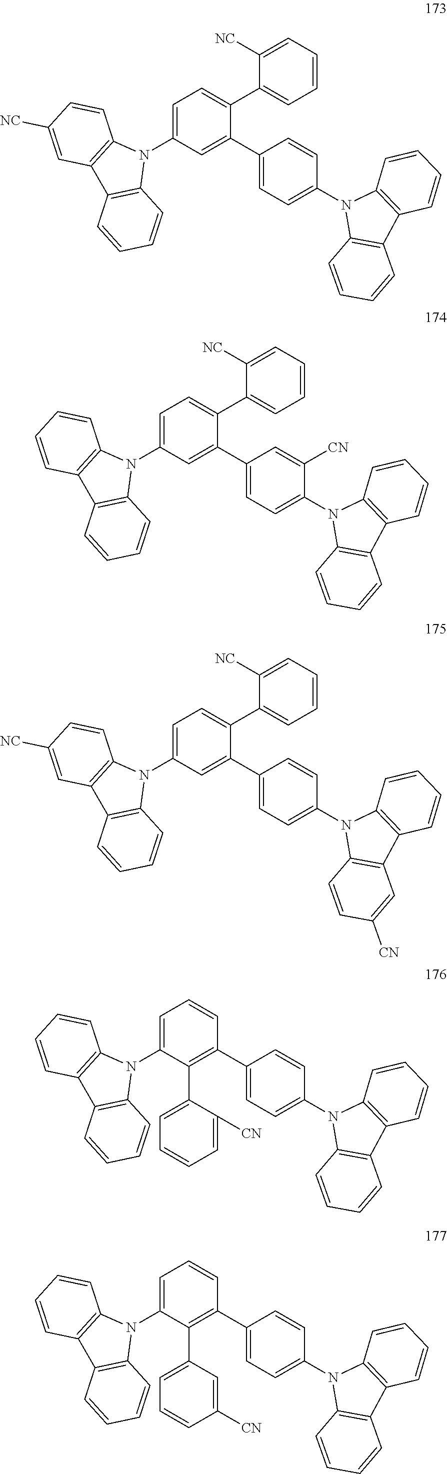 [SMILES: N#Cc1ccc2c(c1)c1ccccc1n2-c1ccc(-c2ccccc2C#N)c(-c2ccc(-n3c4ccccc4c4ccccc43)cc2)c1 ; N#Cc1ccccc1-c1ccc(-n2c3ccccc3c3ccccc32)cc1-c1ccc(-n2c3ccccc3c3ccccc32)c(C#N)c1 ; N#Cc1ccc2c(c1)c1ccccc1n2-c1ccc(-c2cc(-n3c4ccccc4c4cc(C#N)ccc43)ccc2-c2ccccc2C#N)cc1 ; N#Cc1ccccc1-c1c(-c2ccc(-n3c4ccccc4c4ccccc43)cc2)cccc1-n1c2ccccc2c2ccccc21 ; N#Cc1cccc(-c2c(-c3ccc(-n4c5ccccc5c5ccccc54)cc3)cccc2-n2c3ccccc3c3ccccc32)c1]

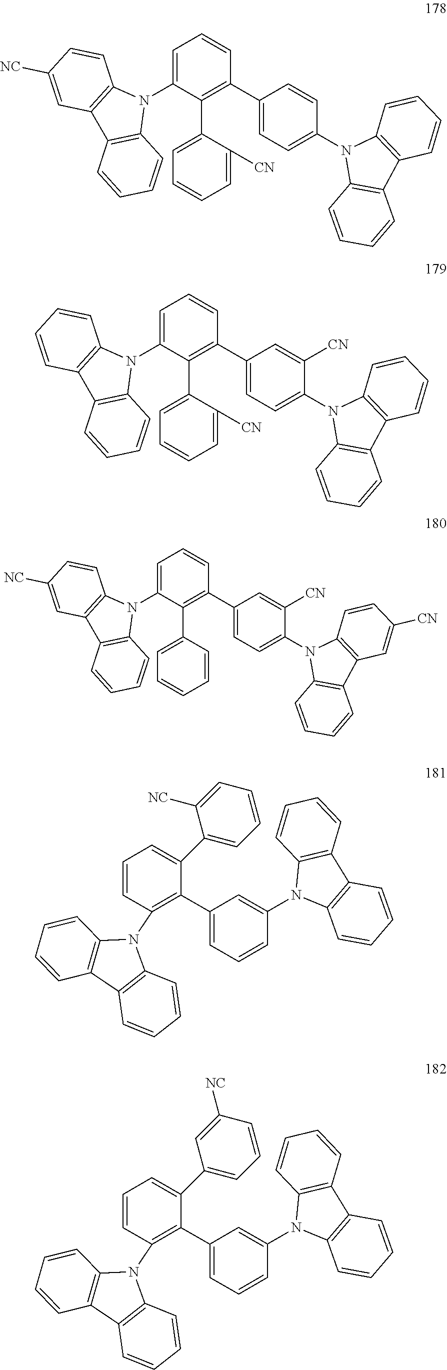

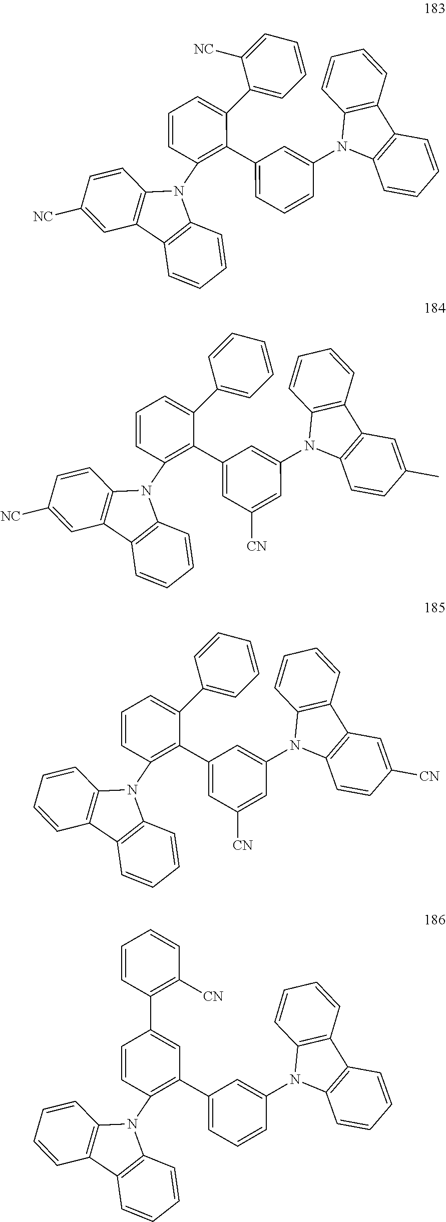

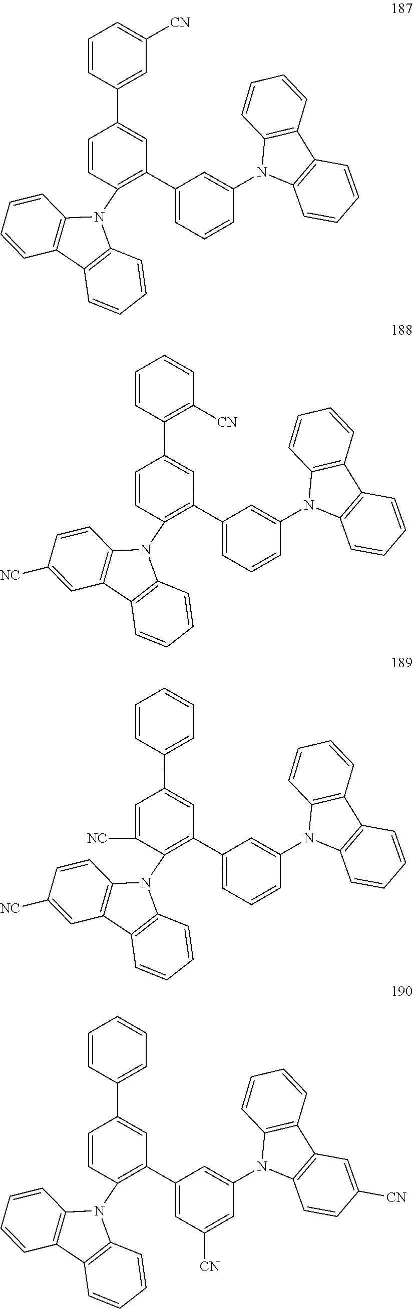

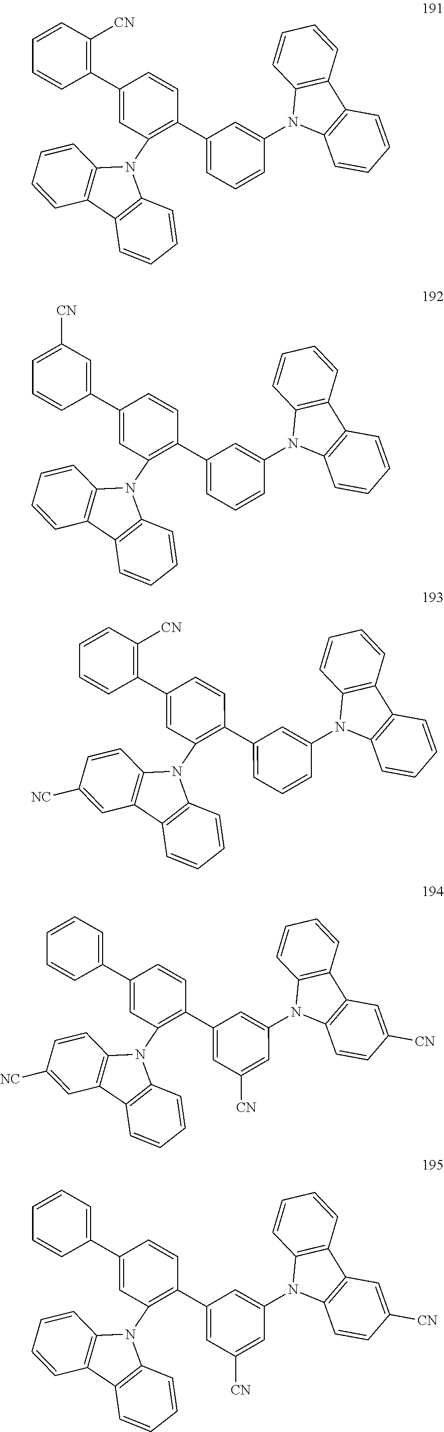

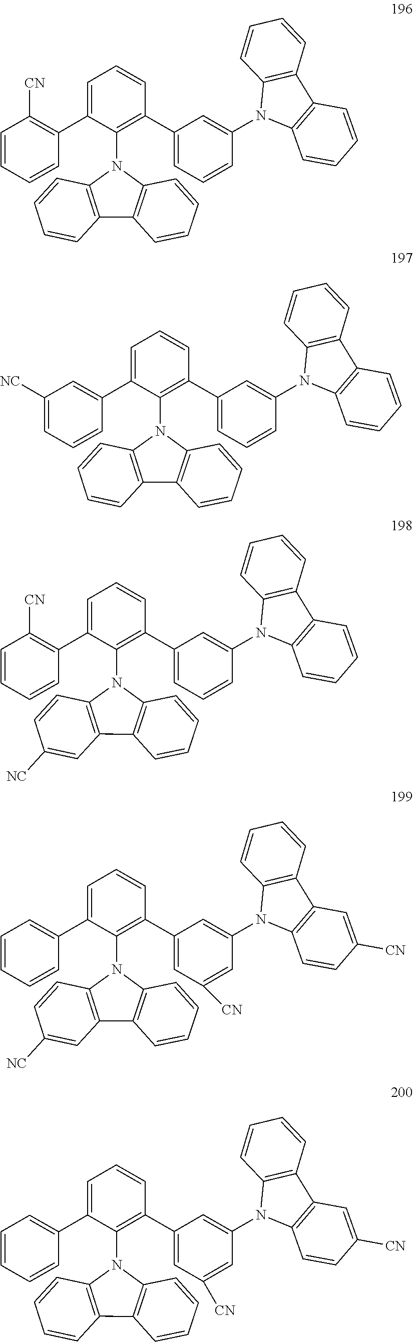

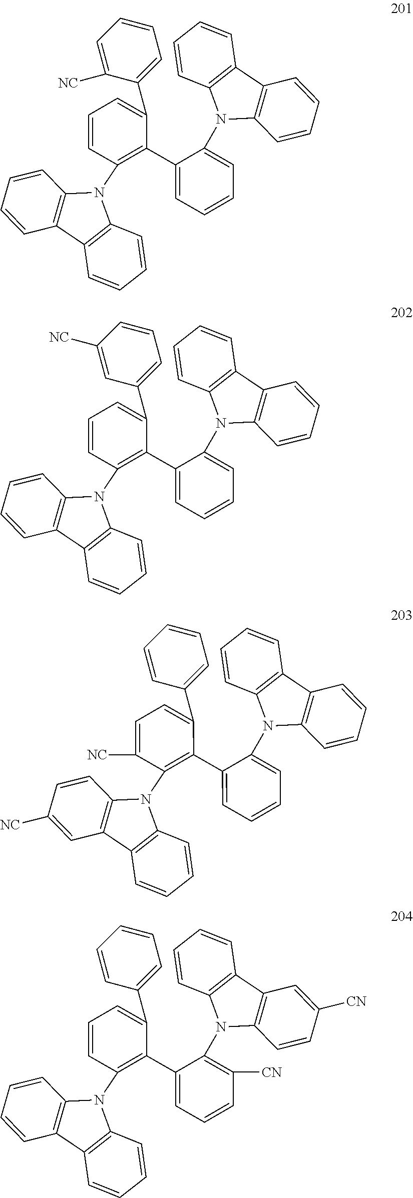

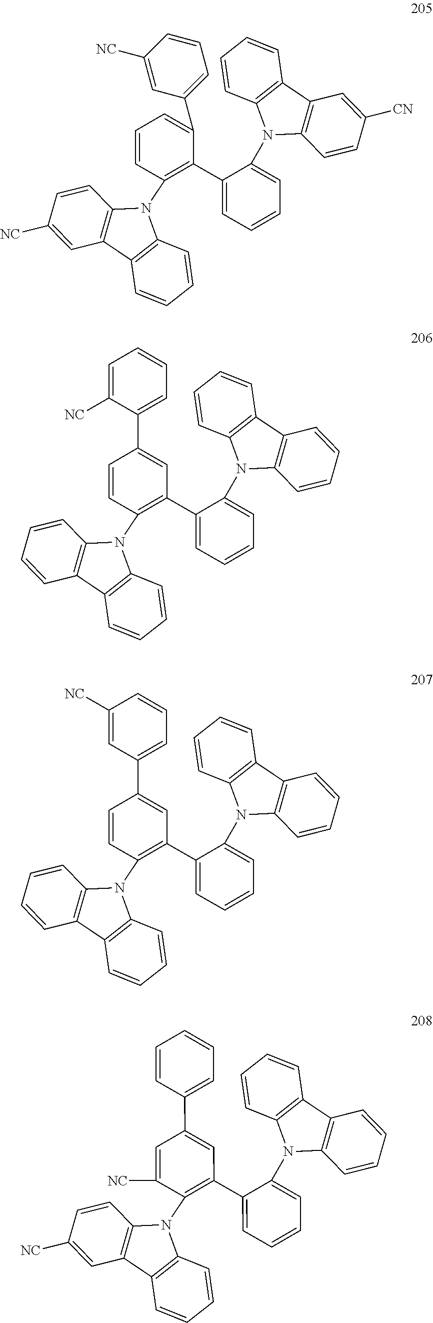 [SMILES: N#Cc1cccc(-c2cccc(-n3c4ccccc4c4cc(C#N)ccc43)c2-c2ccccc2-n2c3ccccc3c3cc(C#N)ccc32)c1 ; N#Cc1ccccc1-c1ccc(-n2c3ccccc3c3ccccc32)c(-c2ccccc2-n2c3ccccc3c3ccccc32)c1 ; N#Cc1cccc(-c2ccc(-n3c4ccccc4c4ccccc43)c(-c3ccccc3-n3c4ccccc4c4ccccc43)c2)c1 ; N#Cc1ccc2c(c1)c1ccccc1n2-c1c(C#N)cc(-c2ccccc2)cc1-c1ccccc1-n1c2ccccc2c2ccccc21]

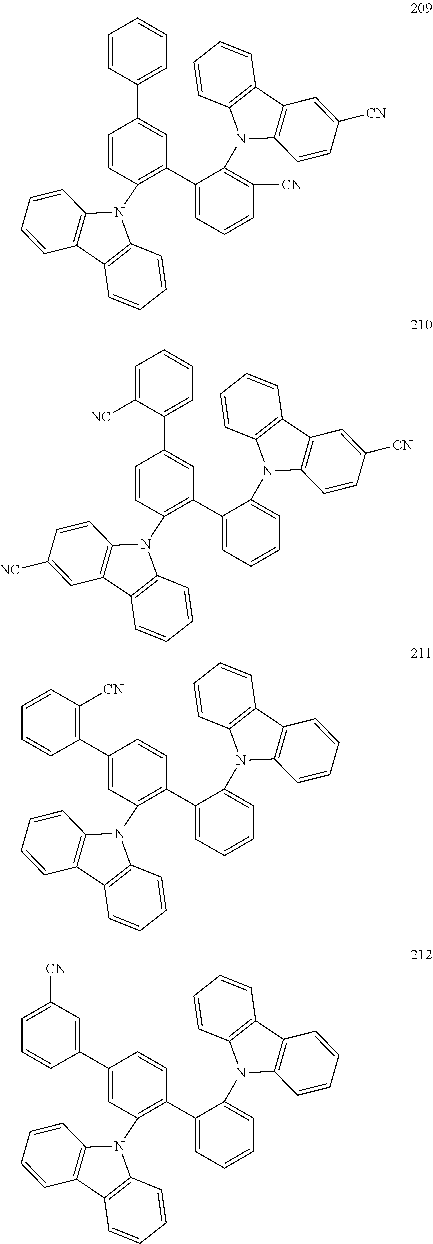 [SMILES: N#Cc1ccc2c(c1)c1ccccc1n2-c1c(C#N)cccc1-c1cc(-c2ccccc2)ccc1-n1c2ccccc2c2ccccc21 ; N#Cc1ccc2c(c1)c1ccccc1n2-c1ccccc1-c1cc(-c2ccccc2C#N)ccc1-n1c2ccccc2c2cc(C#N)ccc21 ; N#Cc1ccccc1-c1ccc(-c2ccccc2-n2c3ccccc3c3ccccc32)c(-n2c3ccccc3c3ccccc32)c1 ; N#Cc1cccc(-c2ccc(-c3ccccc3-n3c4ccccc4c4ccccc43)c(-n3c4ccccc4c4ccccc43)c2)c1]

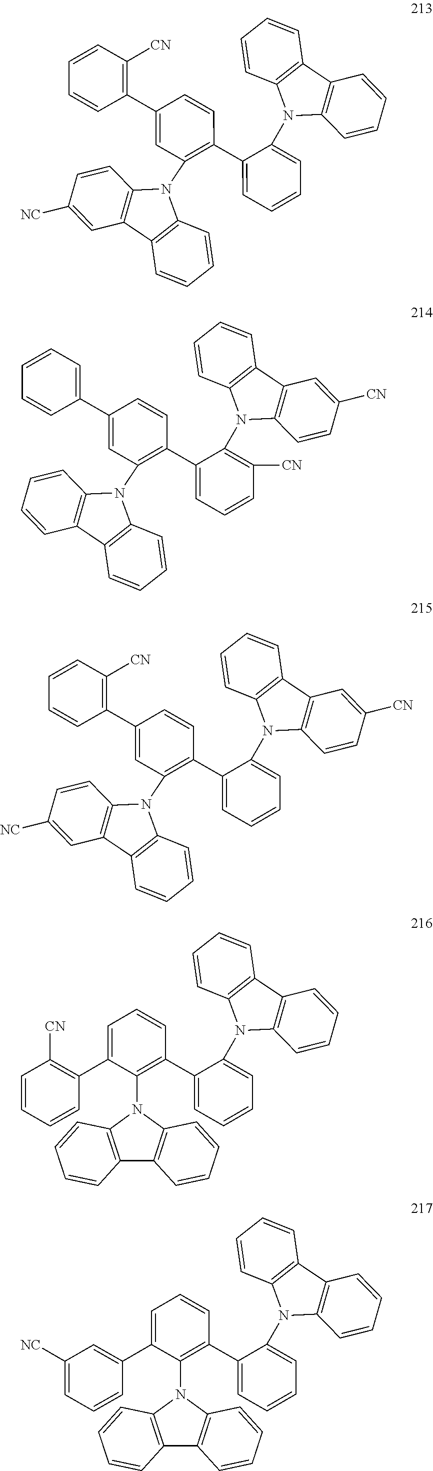 [SMILES: N#Cc1ccc2c(c1)c1ccccc1n2-c1cc(-c2ccccc2C#N)ccc1-c1ccccc1-n1c2ccccc2c2ccccc21 ; N#Cc1ccc2c(c1)c1ccccc1n2-c1c(C#N)cccc1-c1ccc(-c2ccccc2)cc1-n1c2ccccc2c2ccccc21 ; N#Cc1ccc2c(c1)c1ccccc1n2-c1ccccc1-c1ccc(-c2ccccc2C#N)cc1-n1c2ccccc2c2cc(C#N)ccc21 ; N#Cc1ccccc1-c1cccc(-c2ccccc2-n2c3ccccc3c3ccccc32)c1-n1c2ccccc2c2ccccc21 ; N#Cc1cccc(-c2cccc(-c3ccccc3-n3c4ccccc4c4ccccc43)c2-n2c3ccccc3c3ccccc32)c1]

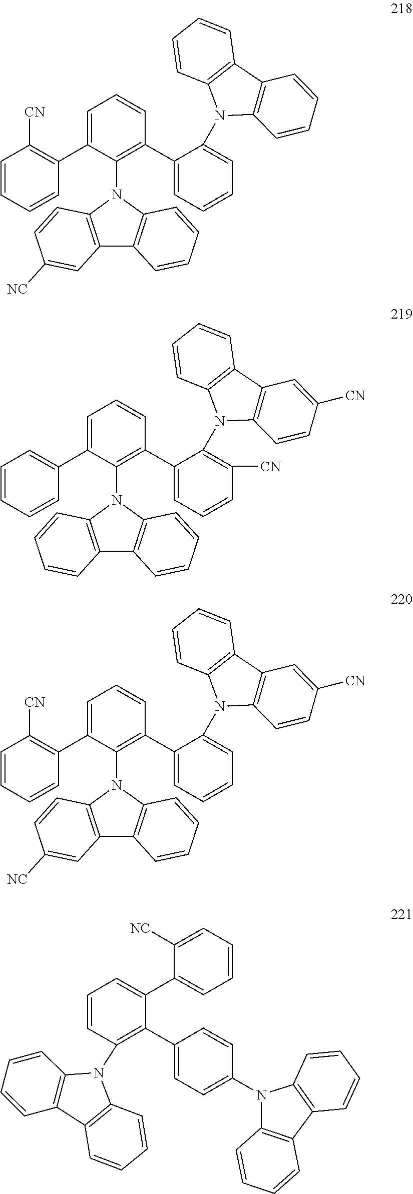

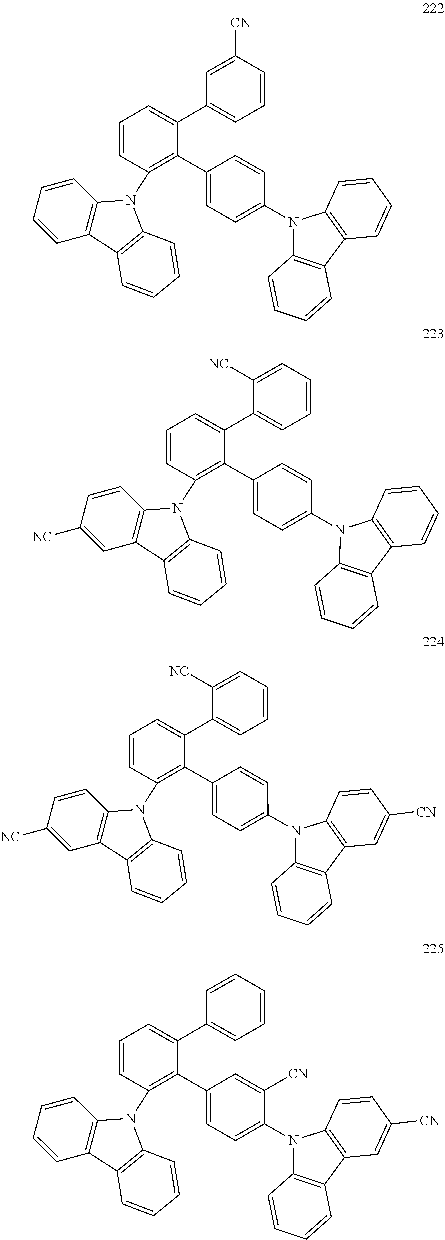 [SMILES: N#Cc1cccc(-c2cccc(-n3c4ccccc4c4ccccc43)c2-c2ccc(-n3c4ccccc4c4ccccc43)cc2)c1 ; N#Cc1ccc2c(c1)c1ccccc1n2-c1cccc(-c2ccccc2C#N)c1-c1ccc(-n2c3ccccc3c3ccccc32)cc1 ; N#Cc1ccc2c(c1)c1ccccc1n2-c1ccc(-c2c(-c3ccccc3C#N)cccc2-n2c3ccccc3c3cc(C#N)ccc32)cc1 ; N#Cc1ccc2c(c1)c1ccccc1n2-c1ccc(-c2c(-c3ccccc3)cccc2-n2c3ccccc3c3ccccc32)cc1C#N]

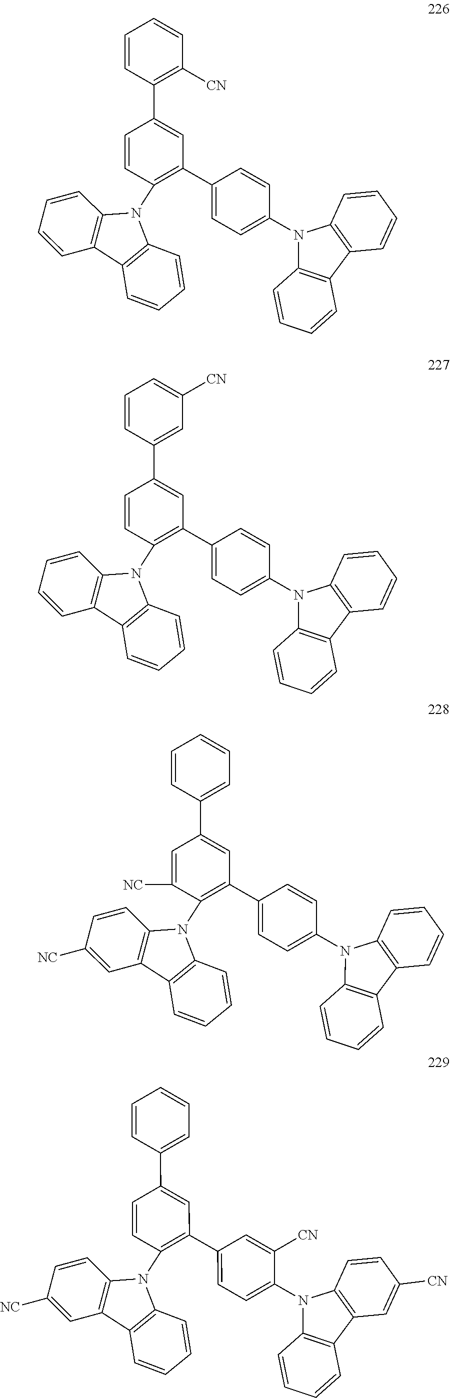 [SMILES: N#Cc1ccccc1-c1ccc(-n2c3ccccc3c3ccccc32)c(-c2ccc(-n3c4ccccc4c4ccccc43)cc2)c1 ; N#Cc1cccc(-c2ccc(-n3c4ccccc4c4ccccc43)c(-c3ccc(-n4c5ccccc5c5ccccc54)cc3)c2)c1 ; N#Cc1ccc2c(c1)c1ccccc1n2-c1c(C#N)cc(-c2ccccc2)cc1-c1ccc(-n2c3ccccc3c3ccccc32)cc1 ; N#Cc1ccc2c(c1)c1ccccc1n2-c1ccc(-c2cc(-c3ccccc3)ccc2-n2c3ccccc3c3cc(C#N)ccc32)cc1C#N]

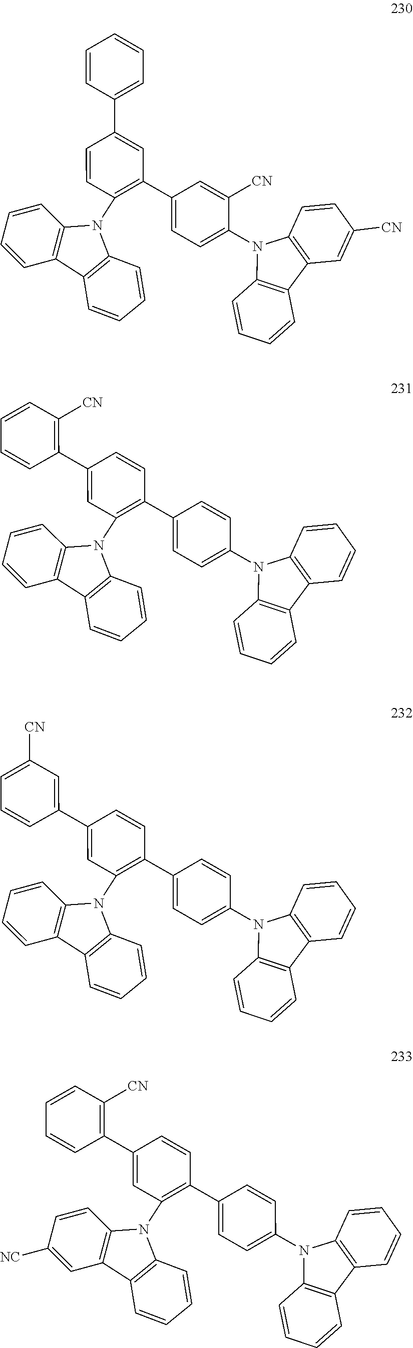 [SMILES: N#Cc1ccc2c(c1)c1ccccc1n2-c1ccc(-c2cc(-c3ccccc3)ccc2-n2c3ccccc3c3ccccc32)cc1C#N ; N#Cc1ccccc1-c1ccc(-c2ccc(-n3c4ccccc4c4ccccc43)cc2)c(-n2c3ccccc3c3ccccc32)c1 ; N#Cc1cccc(-c2ccc(-c3ccc(-n4c5ccccc5c5ccccc54)cc3)c(-n3c4ccccc4c4ccccc43)c2)c1 ; N#Cc1ccc2c(c1)c1ccccc1n2-c1cc(-c2ccccc2C#N)ccc1-c1ccc(-n2c3ccccc3c3ccccc32)cc1]

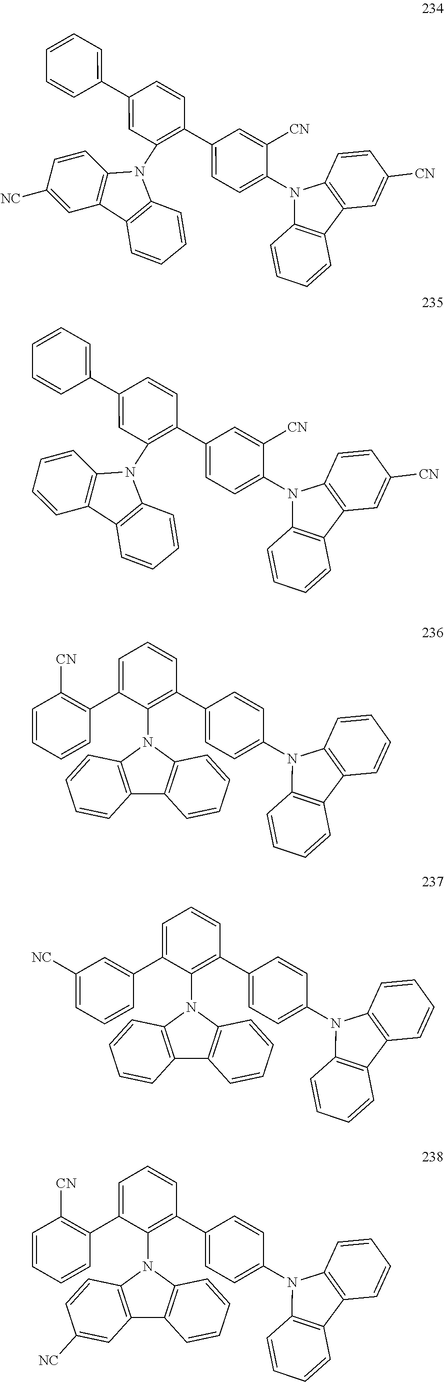 [SMILES: N#Cc1ccc2c(c1)c1ccccc1n2-c1ccc(-c2ccc(-c3ccccc3)cc2-n2c3ccccc3c3cc(C#N)ccc32)cc1C#N ; N#Cc1ccc2c(c1)c1ccccc1n2-c1ccc(-c2ccc(-c3ccccc3)cc2-n2c3ccccc3c3ccccc32)cc1C#N ; N#Cc1ccccc1-c1cccc(-c2ccc(-n3c4ccccc4c4ccccc43)cc2)c1-n1c2ccccc2c2ccccc21 ; N#Cc1cccc(-c2cccc(-c3ccc(-n4c5ccccc5c5ccccc54)cc3)c2-n2c3ccccc3c3ccccc32)c1 ; N#Cc1ccc2c(c1)c1ccccc1n2-c1c(-c2ccc(-n3c4ccccc4c4ccccc43)cc2)cccc1-c1ccccc1C#N]

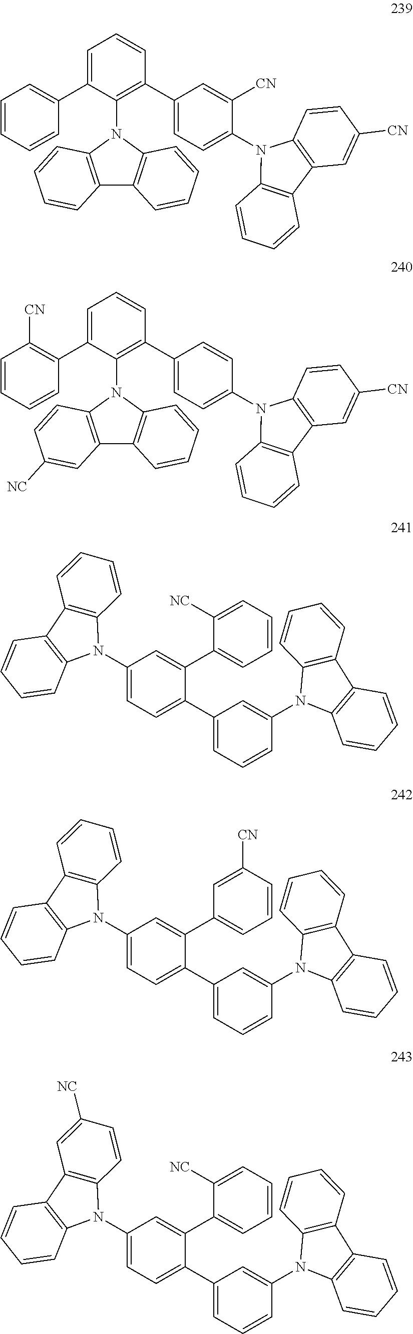 [SMILES: N#Cc1ccc2c(c1)c1ccccc1n2-c1ccc(-c2cccc(-c3ccccc3)c2-n2c3ccccc3c3ccccc32)cc1C#N ; N#Cc1ccc2c(c1)c1ccccc1n2-c1ccc(-c2cccc(-c3ccccc3C#N)c2-n2c3ccccc3c3cc(C#N)ccc32)cc1 ; N#Cc1ccccc1-c1cc(-n2c3ccccc3c3ccccc32)ccc1-c1cccc(-n2c3ccccc3c3ccccc32)c1 ; N#Cc1cccc(-c2cc(-n3c4ccccc4c4ccccc43)ccc2-c2cccc(-n3c4ccccc4c4ccccc43)c2)c1 ; N#Cc1ccc2c(c1)c1ccccc1n2-c1ccc(-c2cccc(-n3c4ccccc4c4ccccc43)c2)c(-c2ccccc2C#N)c1]

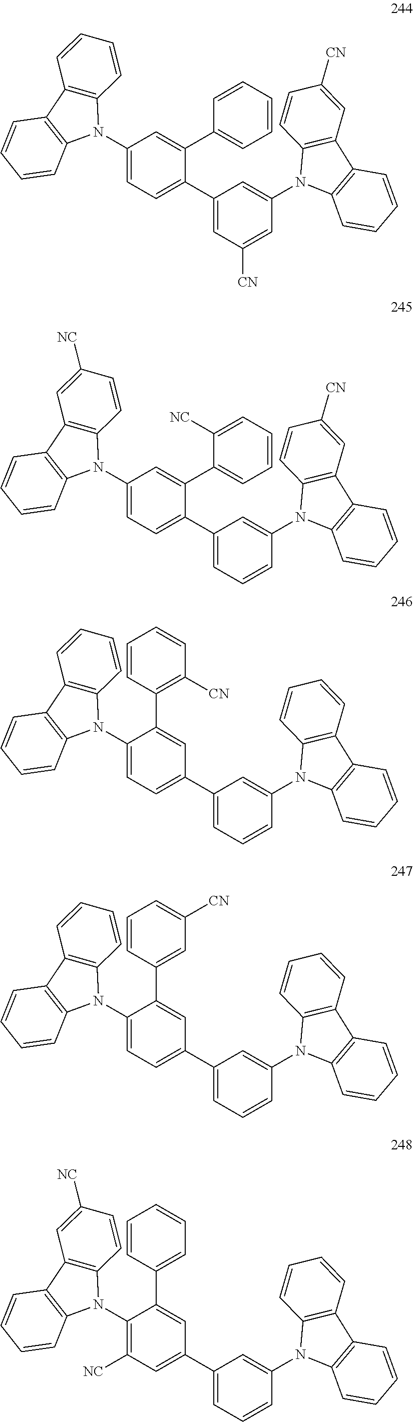 [SMILES: N#Cc1cc(-c2ccc(-n3c4ccccc4c4ccccc43)cc2-c2ccccc2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)cc2-c2ccccc2C#N)c1 ; N#Cc1ccccc1-c1cc(-c2cccc(-n3c4ccccc4c4ccccc43)c2)ccc1-n1c2ccccc2c2ccccc21 ; N#Cc1cccc(-c2cc(-c3cccc(-n4c5ccccc5c5ccccc54)c3)ccc2-n2c3ccccc3c3ccccc32)c1 ; N#Cc1ccc2c(c1)c1ccccc1n2-c1c(C#N)cc(-c2cccc(-n3c4ccccc4c4ccccc43)c2)cc1-c1ccccc1]

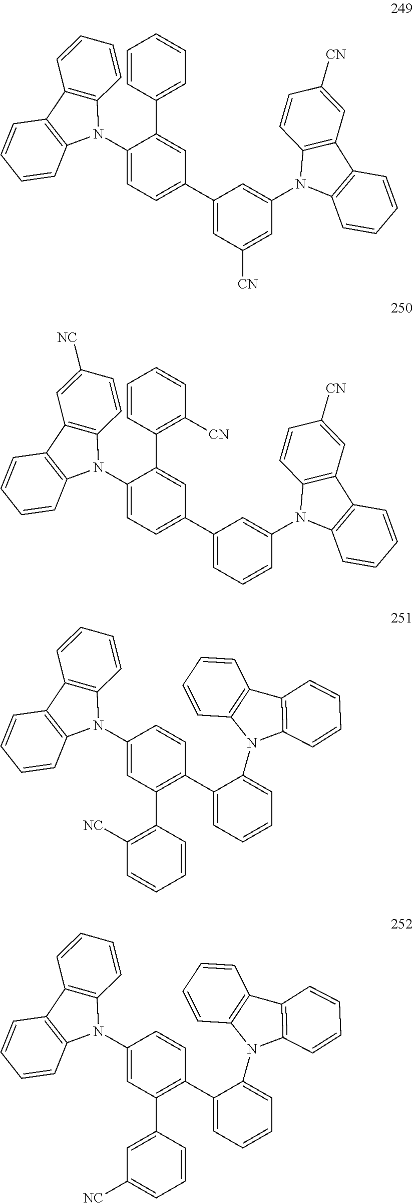 [SMILES: N#Cc1cc(-c2ccc(-n3c4ccccc4c4ccccc43)c(-c3ccccc3)c2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)c(-c3ccccc3C#N)c2)c1 ; N#Cc1ccccc1-c1cc(-n2c3ccccc3c3ccccc32)ccc1-c1ccccc1-n1c2ccccc2c2ccccc21 ; N#Cc1cccc(-c2cc(-n3c4ccccc4c4ccccc43)ccc2-c2ccccc2-n2c3ccccc3c3ccccc32)c1]

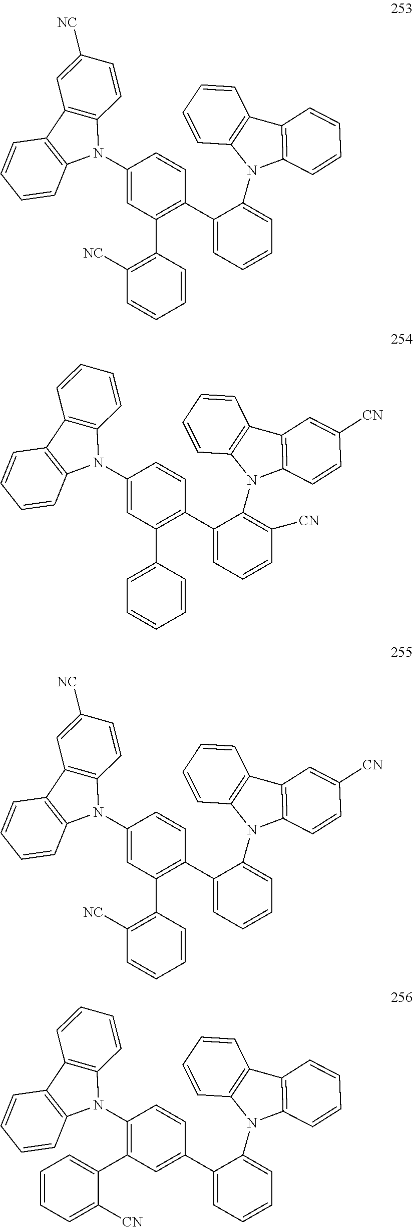 [SMILES: N#Cc1ccc2c(c1)c1ccccc1n2-c1ccc(-c2ccccc2-n2c3ccccc3c3ccccc32)c(-c2ccccc2C#N)c1 ; N#Cc1ccc2c(c1)c1ccccc1n2-c1c(C#N)cccc1-c1ccc(-n2c3ccccc3c3ccccc32)cc1-c1ccccc1 ; N#Cc1ccc2c(c1)c1ccccc1n2-c1ccc(-c2ccccc2-n2c3ccccc3c3cc(C#N)ccc32)c(-c2ccccc2C#N)c1 ; N#Cc1ccccc1-c1cc(-c2ccccc2-n2c3ccccc3c3ccccc32)ccc1-n1c2ccccc2c2ccccc21]

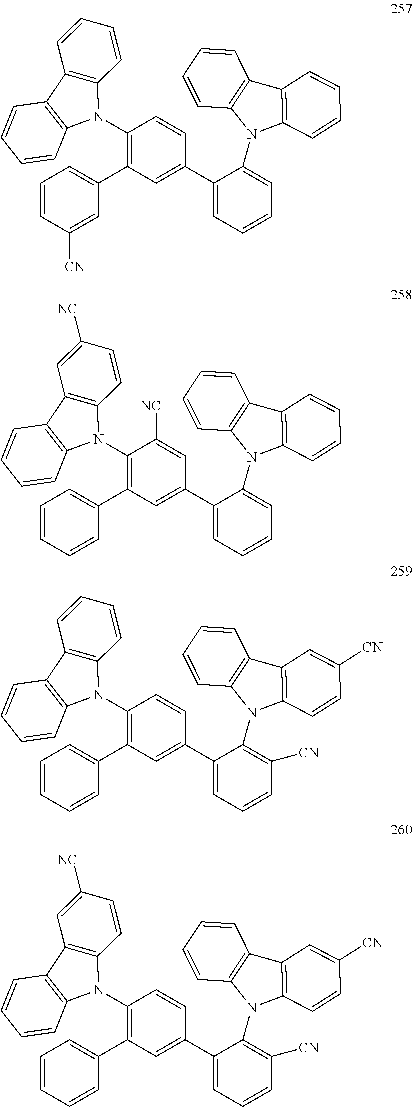

In one or more embodiments, the hole-transporting host may be one of Compounds H-H1 to H-H104, but embodiments of the present disclosure are not limited thereto:

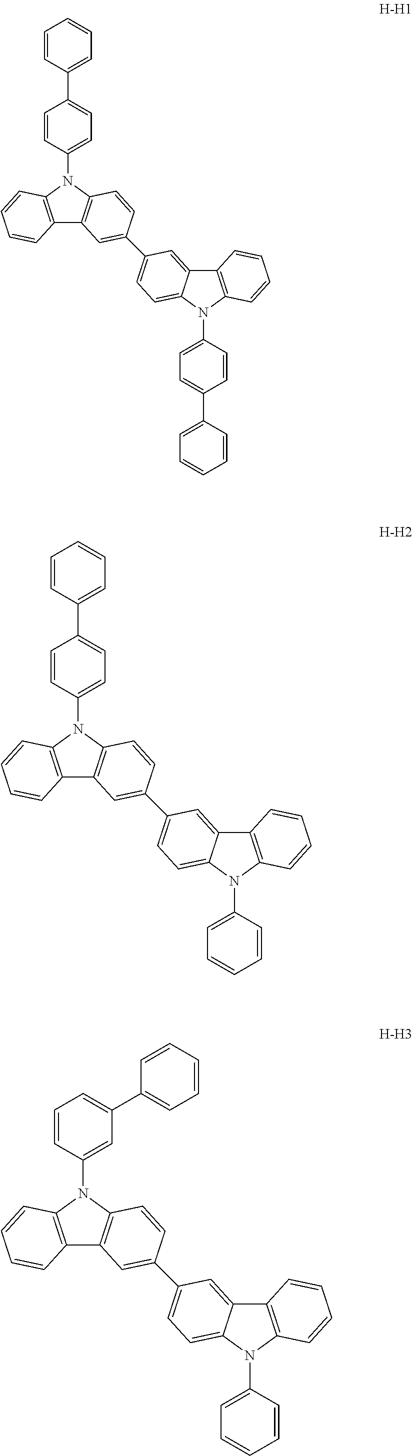 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

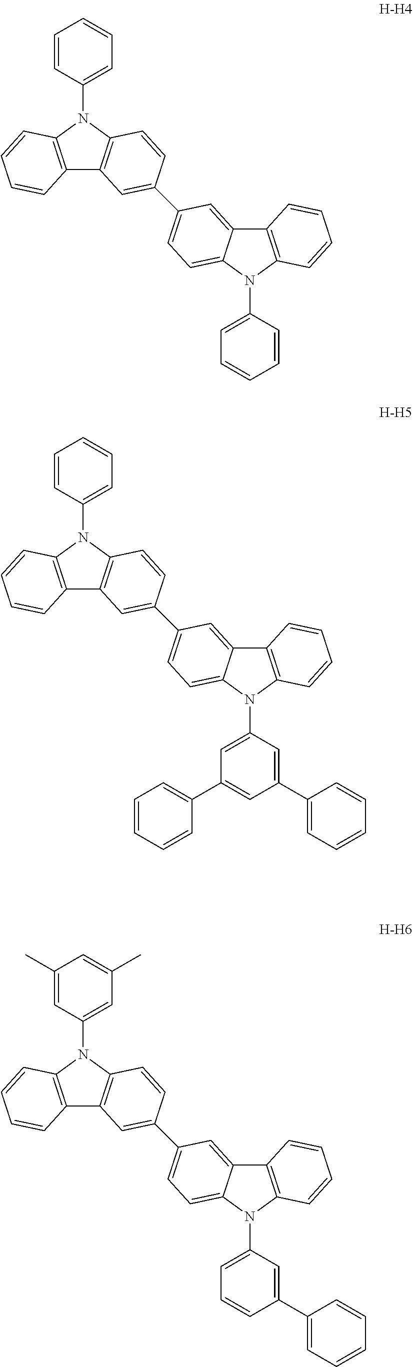 [SMILES: c1ccc(-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; Cc1cc(C)cc(-n2c3ccccc3c3cc(-c4ccc5c(c4)c4ccccc4n5-c4cccc(-c5ccccc5)c4)ccc32)c1]

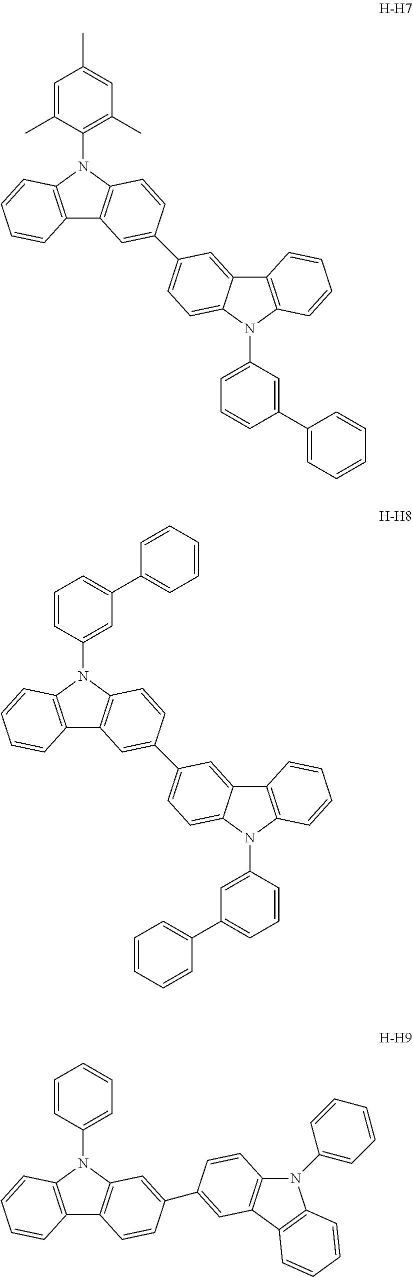 [SMILES: Cc1cc(C)c(-n2c3ccccc3c3cc(-c4ccc5c(c4)c4ccccc4n5-c4cccc(-c5ccccc5)c4)ccc32)c(C)c1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c6ccccc6n(-c6ccccc6)c5c4)ccc32)cc1]

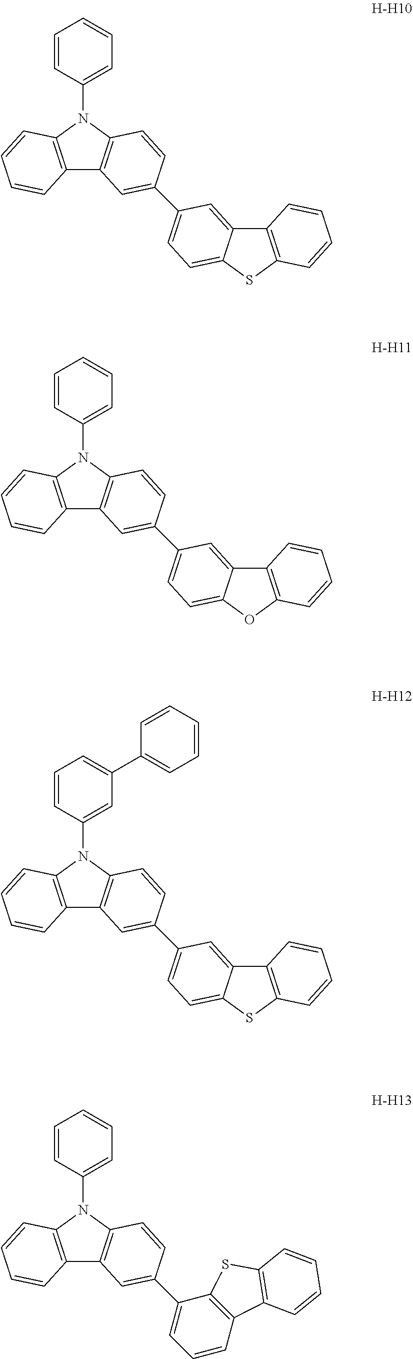 [SMILES: c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1 ; c1ccc(-c2cccc(-n3c4ccccc4c4cc(-c5ccc6sc7ccccc7c6c5)ccc43)c2)cc1 ; c1ccc(-n2c3ccccc3c3cc(-c4cccc5c4sc4ccccc45)ccc32)cc1]

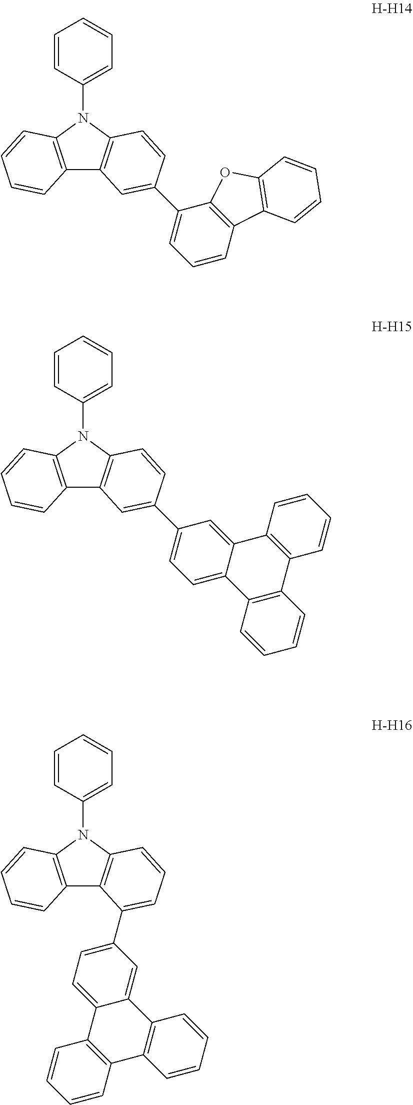 [SMILES: c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1 ; c1ccc(-n2c3ccccc3c3c(-c4ccc5c6ccccc6c6ccccc6c5c4)cccc32)cc1]

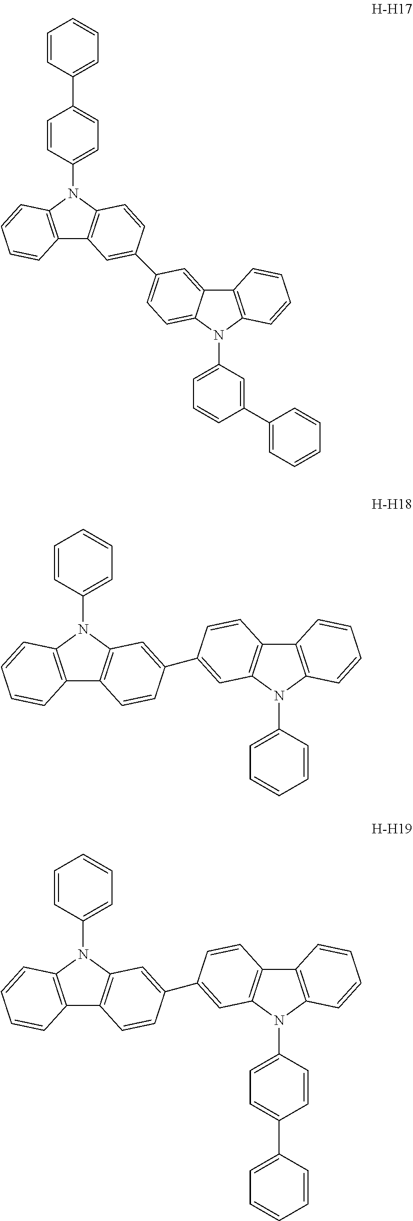 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3ccc(-c4ccc5c6ccccc6n(-c6ccccc6)c5c4)cc32)cc1 ; c1ccc(-c2ccc(-n3c4ccccc4c4ccc(-c5ccc6c7ccccc7n(-c7ccccc7)c6c5)cc43)cc2)cc1]

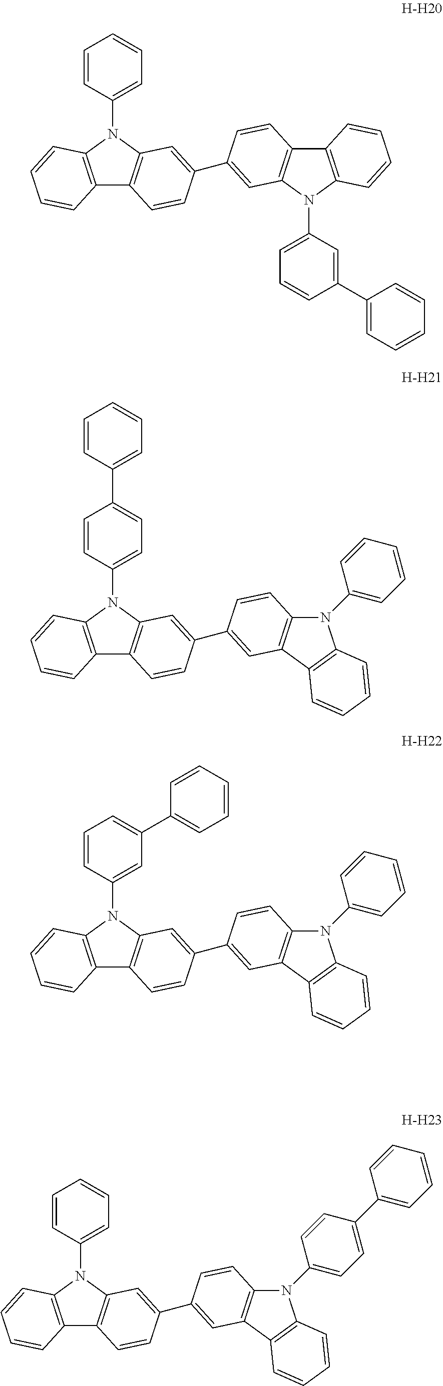 [SMILES: c1ccc(-c2cccc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c(c5)c5ccccc5n6-c5ccccc5)cc43)cc2)cc1 ; c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1]

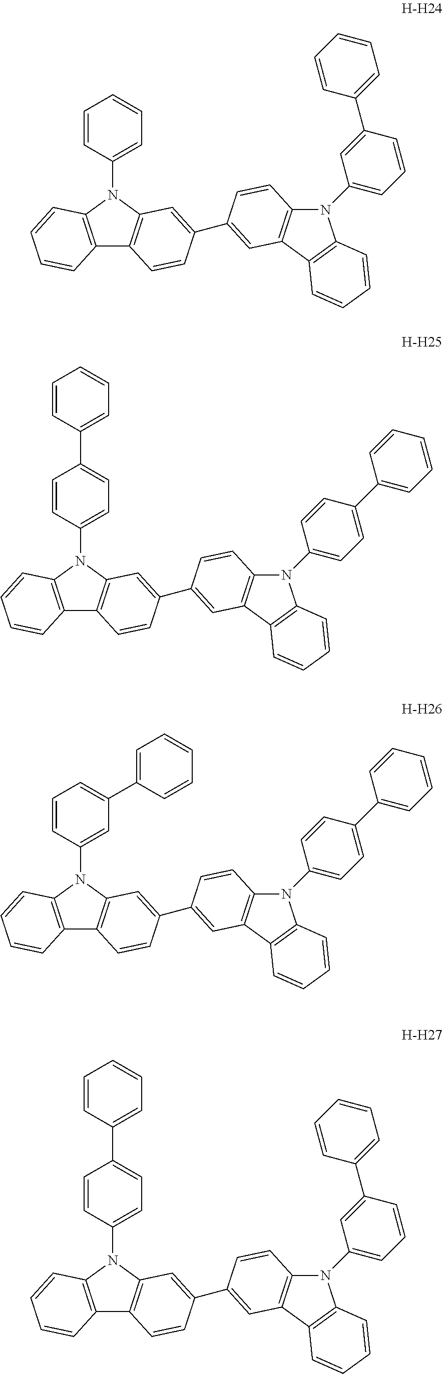 [SMILES: c1ccc(-c2cccc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1]

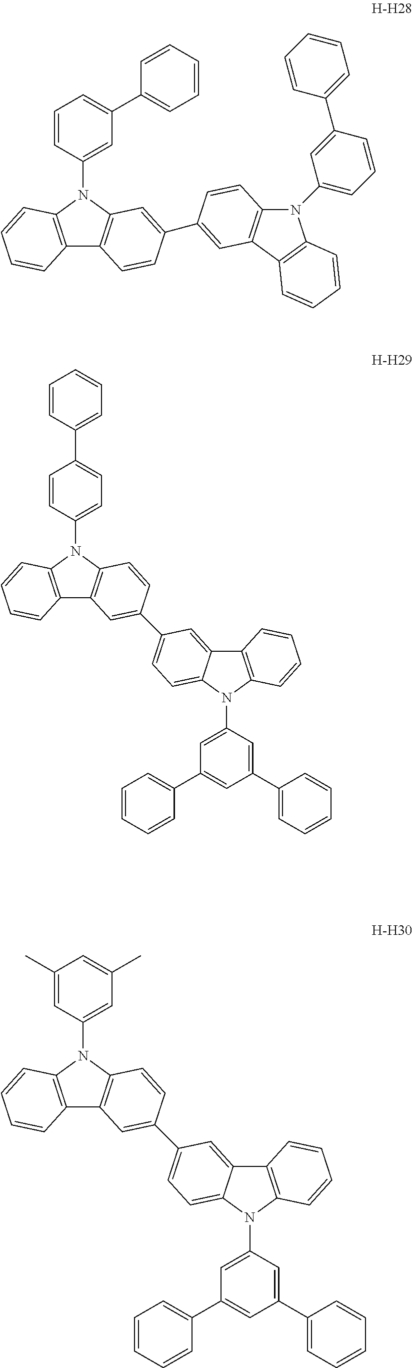 [SMILES: c1ccc(-c2cccc(-n3c4ccccc4c4cc(-c5ccc6c7ccccc7n(-c7cccc(-c8ccccc8)c7)c6c5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; Cc1cc(C)cc(-n2c3ccccc3c3cc(-c4ccc5c(c4)c4ccccc4n5-c4cc(-c5ccccc5)cc(-c5ccccc5)c4)ccc32)c1]

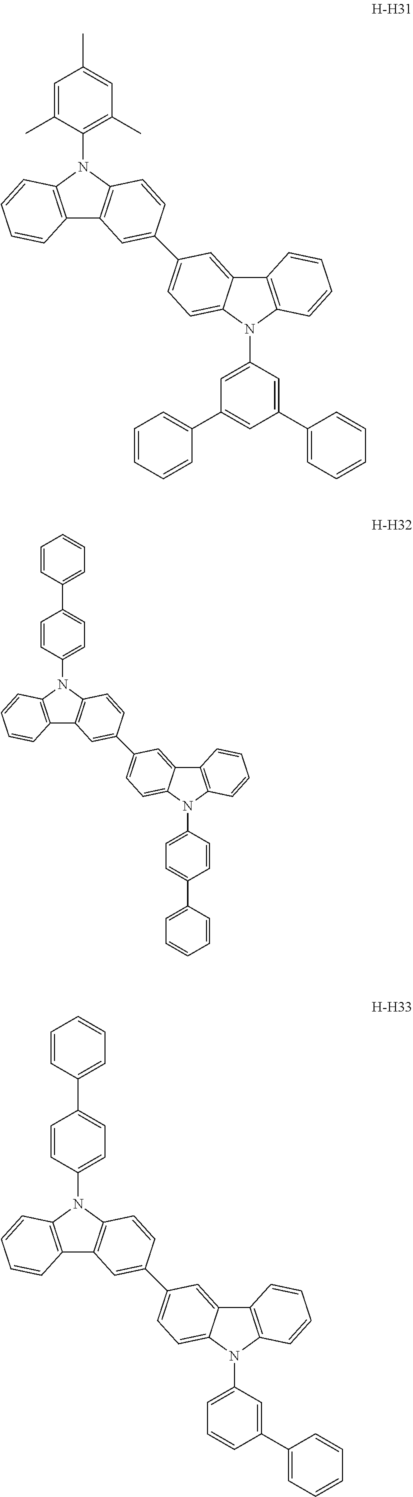 [SMILES: Cc1cc(C)c(-n2c3ccccc3c3cc(-c4ccc5c(c4)c4ccccc4n5-c4cc(-c5ccccc5)cc(-c5ccccc5)c4)ccc32)c(C)c1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

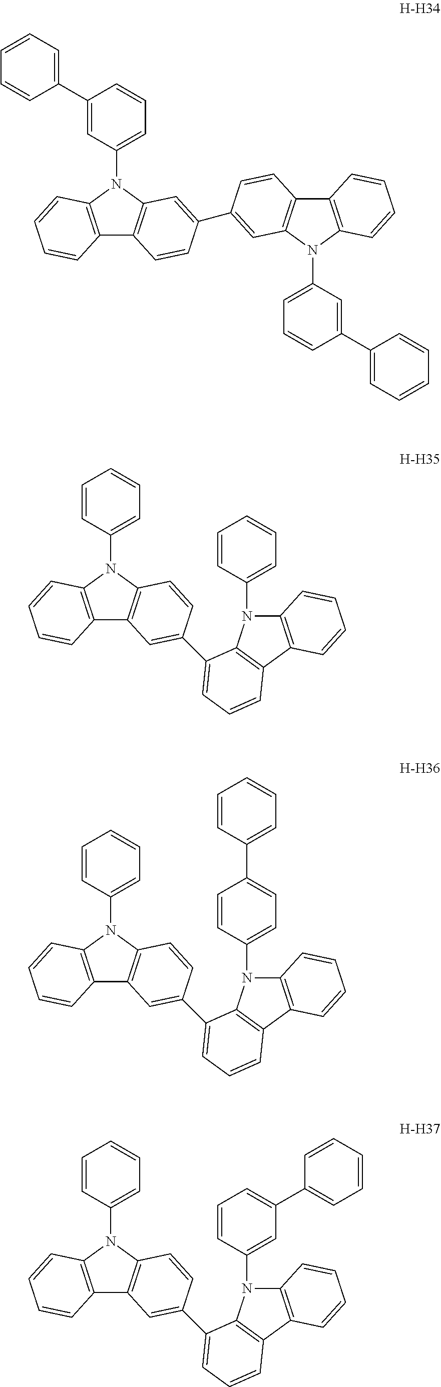 [SMILES: c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1 ; c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1]

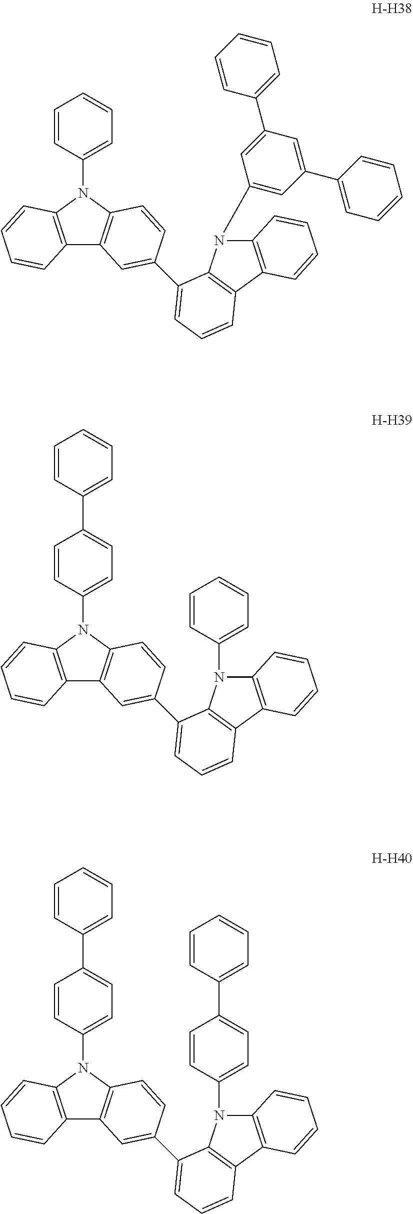 [SMILES: c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1 ; c1ccc(-c2ccc(-n3c4ccccc4c4cc(-c5cccc6c7ccccc7n(-c7ccc(-c8ccccc8)cc7)c56)ccc43)cc2)cc1]

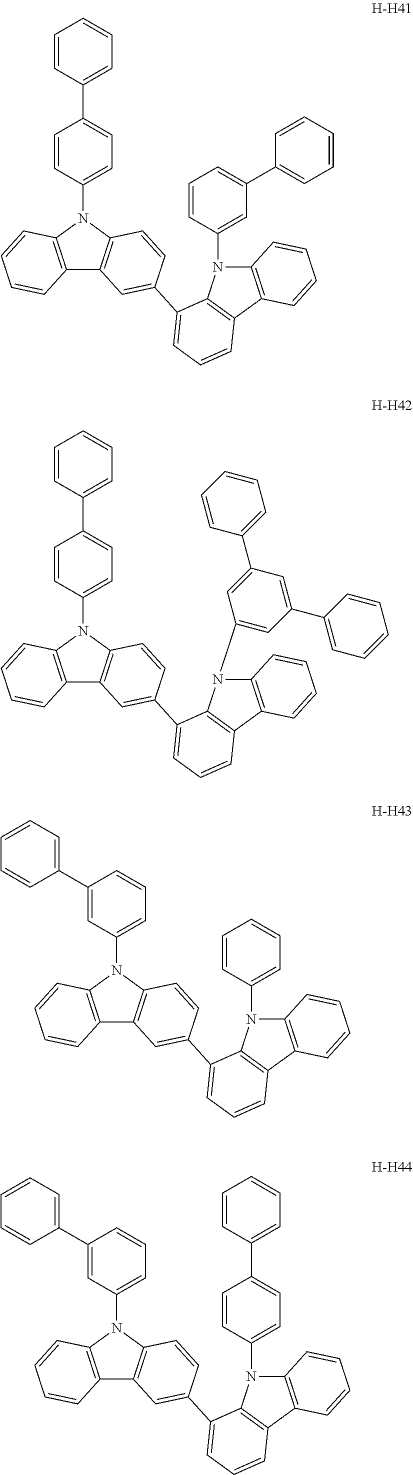 [SMILES: c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1 ; c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1]

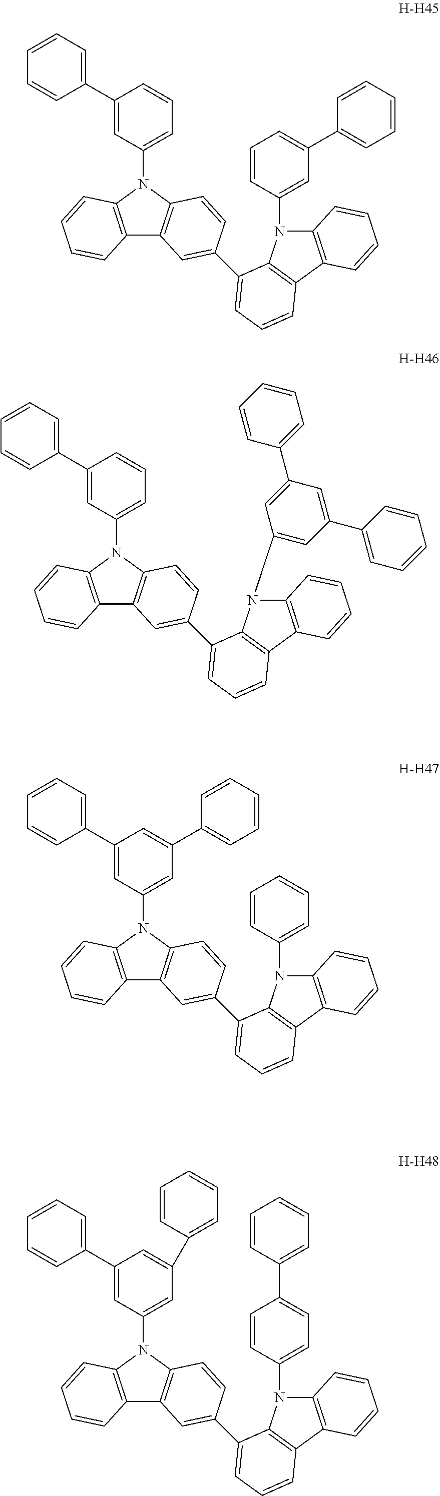 [SMILES: c1ccc(-c2cccc(-n3c4ccccc4c4cc(-c5cccc6c7ccccc7n(-c7cccc(-c8ccccc8)c7)c56)ccc43)c2)cc1 ; c1ccc(-c2cccc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cc(-c6ccccc6)cc(-c6ccccc6)c5)c43)cc2)cc1]

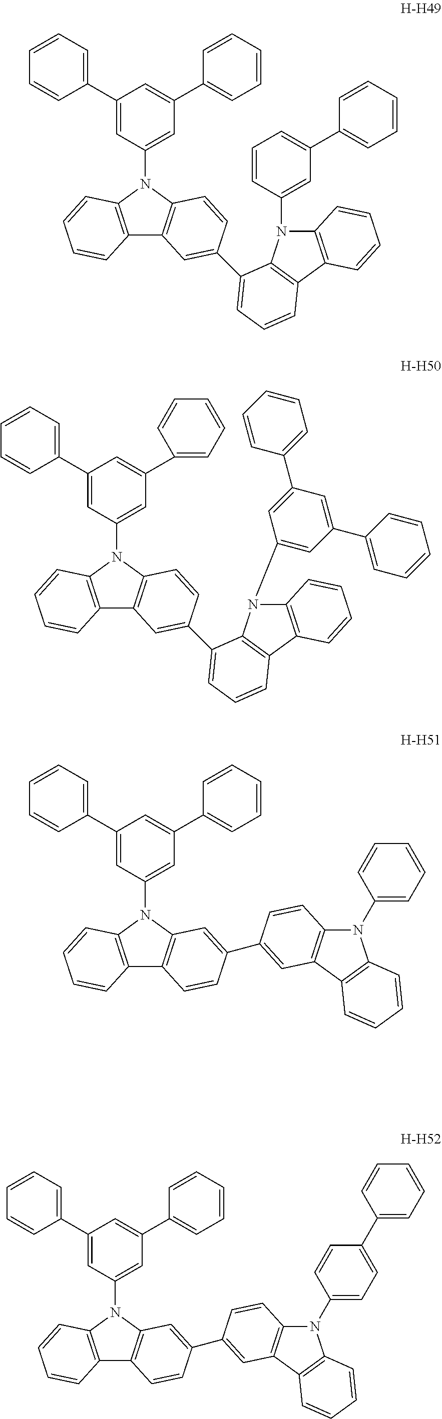 [SMILES: c1ccc(-c2cc(-c3ccccc3)cc(-n3c4ccccc4c4cc(-c5cccc6c7ccccc7n(-c7cccc(-c8ccccc8)c7)c56)ccc43)c2)cc1 ; c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1]

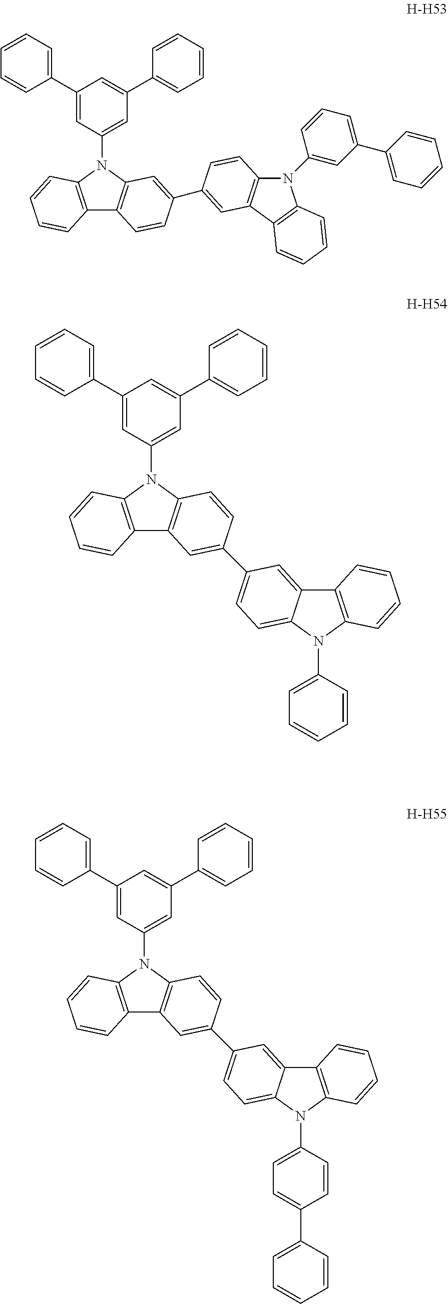 [SMILES: c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

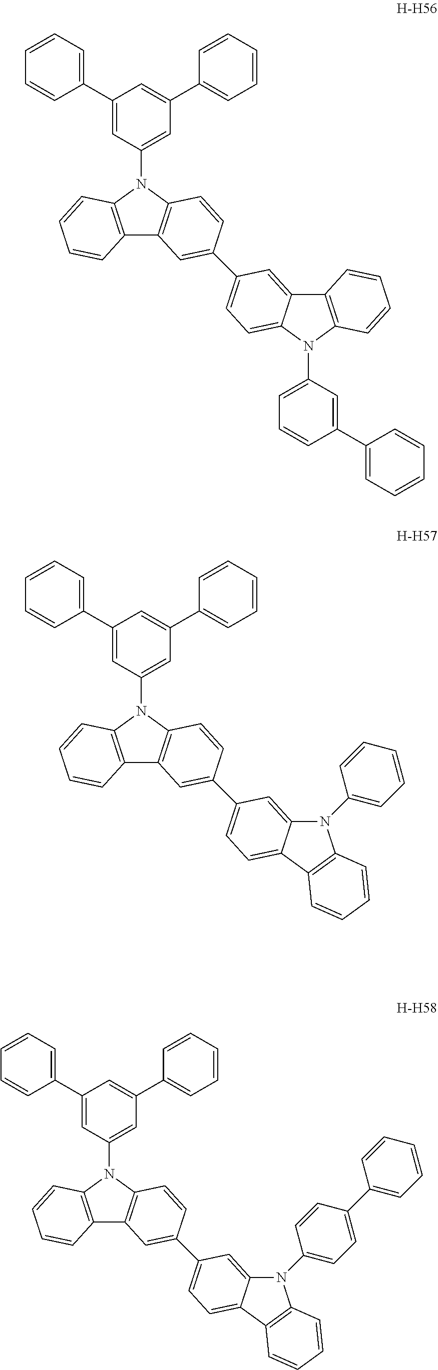 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1]

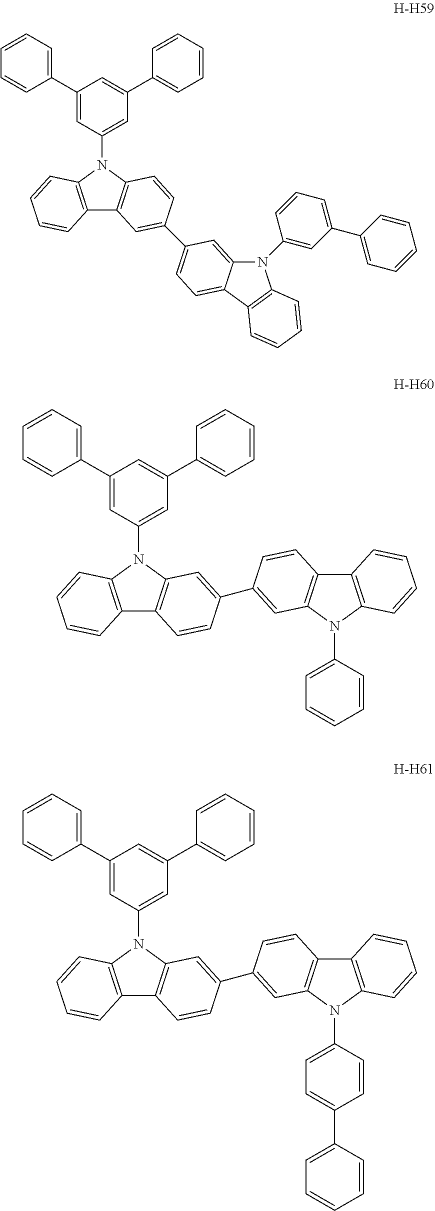 [SMILES: c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1 ; c1ccc(-c2cc(-c3ccccc3)cc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1]

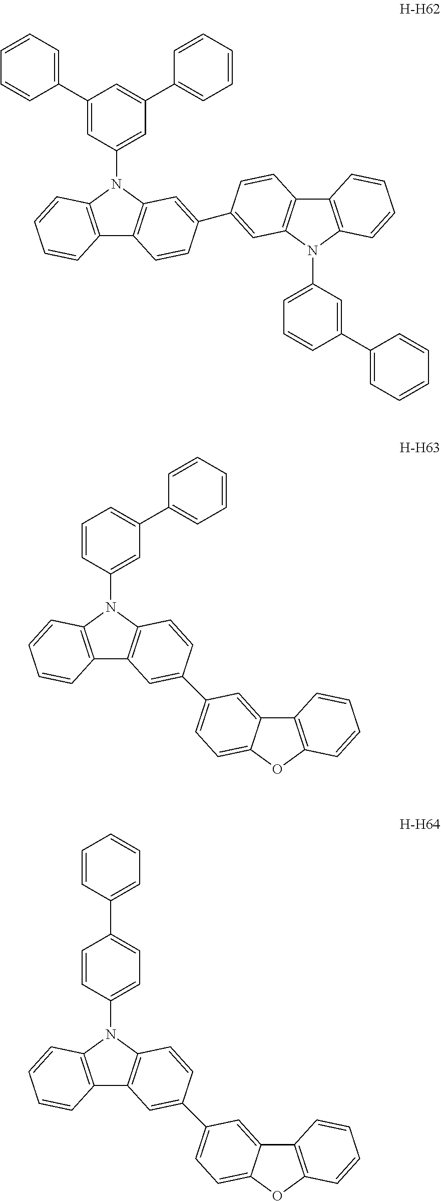 [SMILES: c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-c2cccc(-n3c4ccccc4c4cc(-c5ccc6oc7ccccc7c6c5)ccc43)c2)cc1 ; c1ccc(-c2ccc(-n3c4ccccc4c4cc(-c5ccc6oc7ccccc7c6c5)ccc43)cc2)cc1]

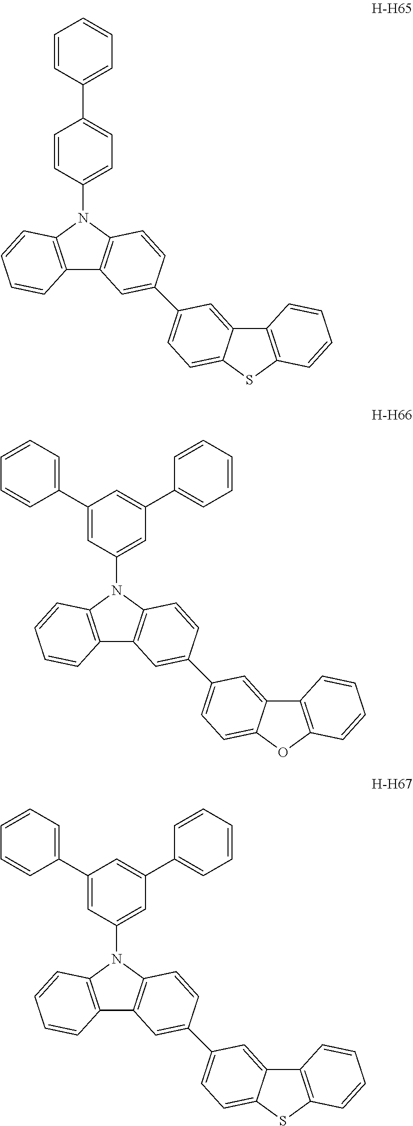 [SMILES: c1ccc(-c2ccc(-n3c4ccccc4c4cc(-c5ccc6sc7ccccc7c6c5)ccc43)cc2)cc1 ; c1ccc(-c2cc(-c3ccccc3)cc(-n3c4ccccc4c4cc(-c5ccc6oc7ccccc7c6c5)ccc43)c2)cc1 ; c1ccc(-c2cc(-c3ccccc3)cc(-n3c4ccccc4c4cc(-c5ccc6sc7ccccc7c6c5)ccc43)c2)cc1]

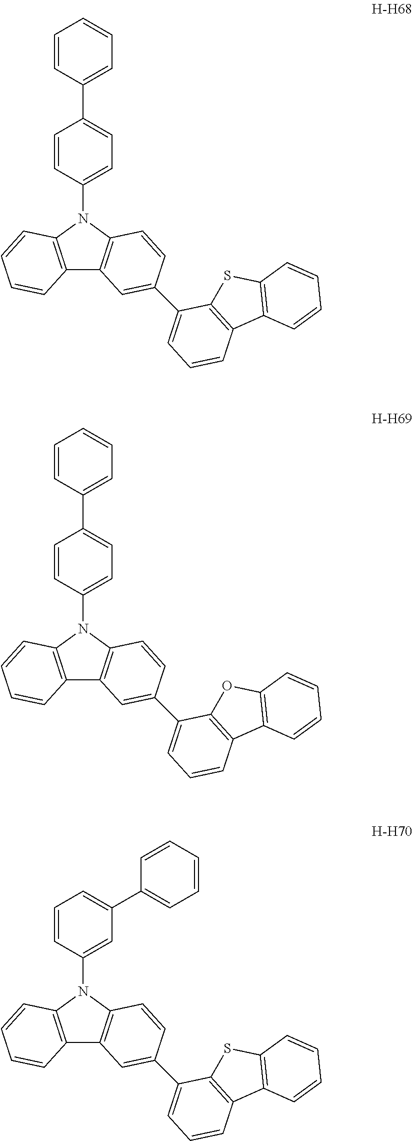 [SMILES: c1ccc(-c2ccc(-n3c4ccccc4c4cc(-c5cccc6c5sc5ccccc56)ccc43)cc2)cc1 ; c1ccc(-c2ccc(-n3c4ccccc4c4cc(-c5cccc6c5oc5ccccc56)ccc43)cc2)cc1 ; c1ccc(-c2cccc(-n3c4ccccc4c4cc(-c5cccc6c5sc5ccccc56)ccc43)c2)cc1]

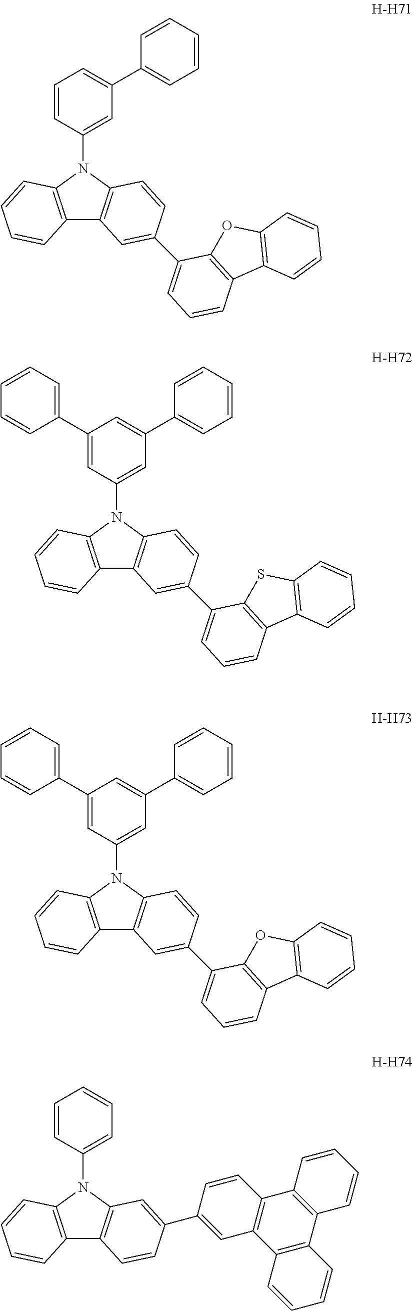 [SMILES: c1ccc(-c2cccc(-n3c4ccccc4c4cc(-c5cccc6c5oc5ccccc56)ccc43)c2)cc1 ; c1ccc(-c2cc(-c3ccccc3)cc(-n3c4ccccc4c4cc(-c5cccc6c5sc5ccccc56)ccc43)c2)cc1 ; c1ccc(-c2cc(-c3ccccc3)cc(-n3c4ccccc4c4cc(-c5cccc6c5oc5ccccc56)ccc43)c2)cc1 ; c1ccc(-n2c3ccccc3c3ccc(-c4ccc5c6ccccc6c6ccccc6c5c4)cc32)cc1]

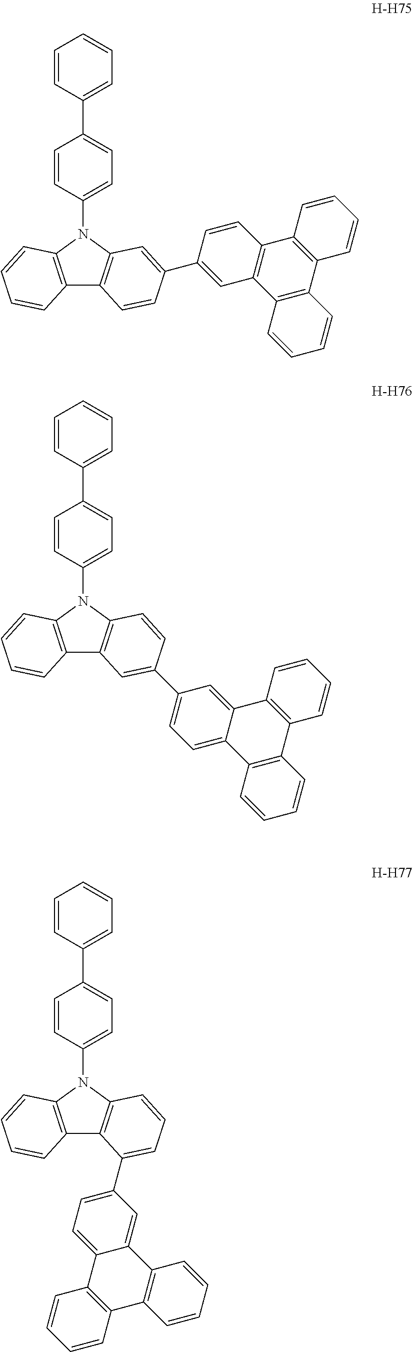 [SMILES: c1ccc(-c2ccc(-n3c4ccccc4c4ccc(-c5ccc6c7ccccc7c7ccccc7c6c5)cc43)cc2)cc1 ; c1ccc(-c2ccc(-n3c4ccccc4c4cc(-c5ccc6c7ccccc7c7ccccc7c6c5)ccc43)cc2)cc1 ; c1ccc(-c2ccc(-n3c4ccccc4c4c(-c5ccc6c7ccccc7c7ccccc7c6c5)cccc43)cc2)cc1]

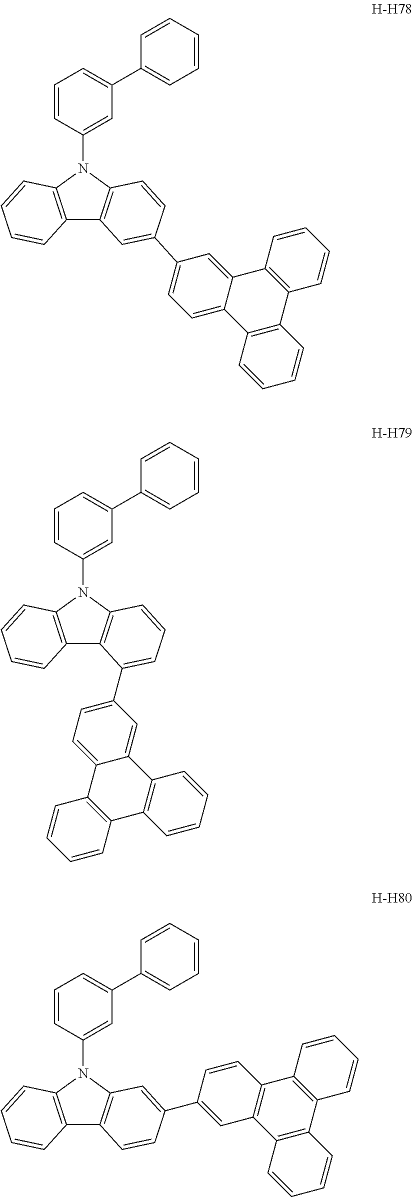 [SMILES: c1ccc(-c2cccc(-n3c4ccccc4c4cc(-c5ccc6c7ccccc7c7ccccc7c6c5)ccc43)c2)cc1 ; c1ccc(-c2cccc(-n3c4ccccc4c4c(-c5ccc6c7ccccc7c7ccccc7c6c5)cccc43)c2)cc1 ; c1ccc(-c2cccc(-n3c4ccccc4c4ccc(-c5ccc6c7ccccc7c7ccccc7c6c5)cc43)c2)cc1]

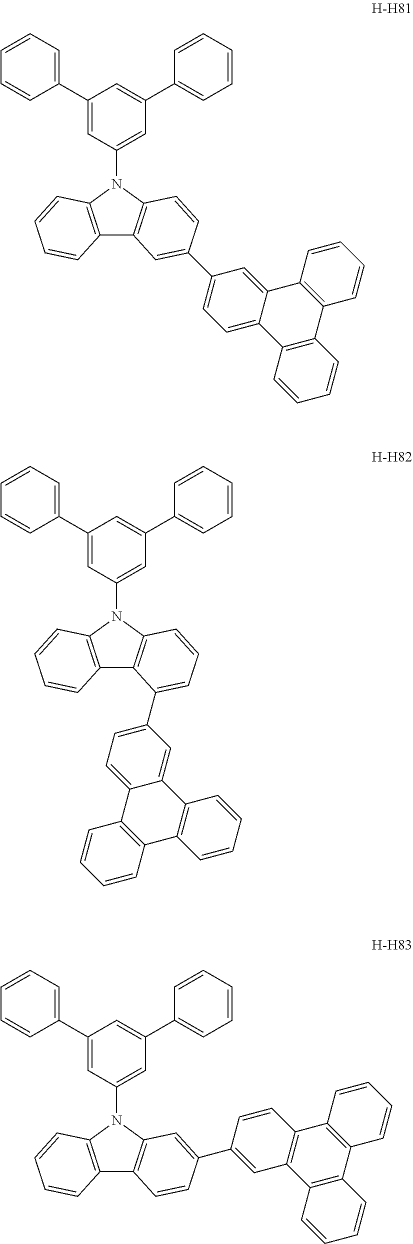 [SMILES: c1ccc(-c2cc(-c3ccccc3)cc(-n3c4ccccc4c4cc(-c5ccc6c7ccccc7c7ccccc7c6c5)ccc43)c2)cc1 ; c1ccc(-c2cc(-c3ccccc3)cc(-n3c4ccccc4c4c(-c5ccc6c7ccccc7c7ccccc7c6c5)cccc43)c2)cc1 ; c1ccc(-c2cc(-c3ccccc3)cc(-n3c4ccccc4c4ccc(-c5ccc6c7ccccc7c7ccccc7c6c5)cc43)c2)cc1]

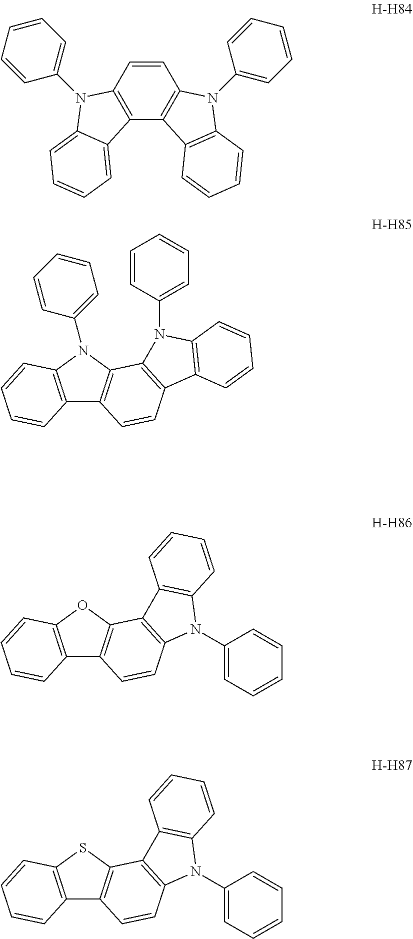 [SMILES: c1ccc(-n2c3ccccc3c3c4c5ccccc5n(-c5ccccc5)c4ccc32)cc1 ; c1ccc(-n2c3ccccc3c3ccc4c5ccccc5n(-c5ccccc5)c4c32)cc1 ; c1ccc(-n2c3ccccc3c3c4oc5ccccc5c4ccc32)cc1 ; c1ccc(-n2c3ccccc3c3c4sc5ccccc5c4ccc32)cc1]

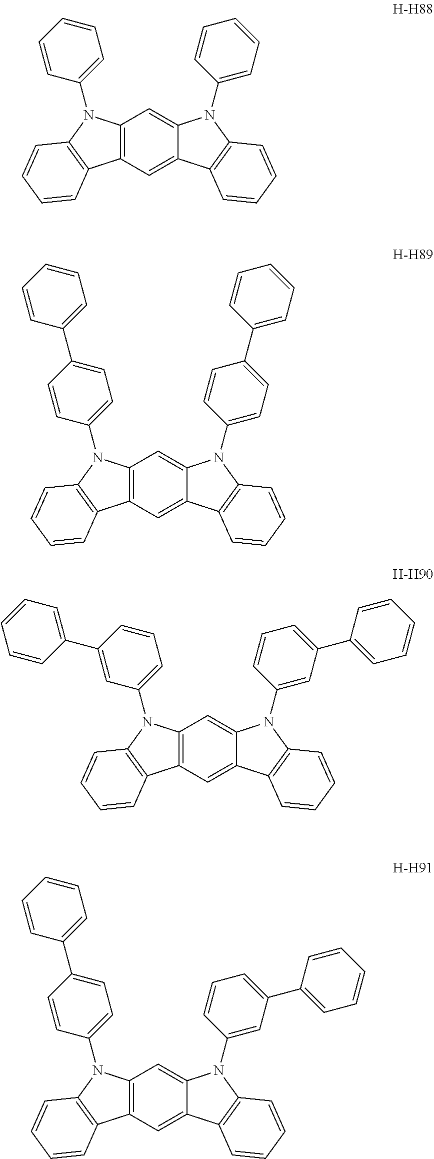 [SMILES: c1ccc(-n2c3ccccc3c3cc4c5ccccc5n(-c5ccccc5)c4cc32)cc1 ; c1ccc(-c2ccc(-n3c4ccccc4c4cc5c6ccccc6n(-c6ccc(-c7ccccc7)cc6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-c2ccc(-n3c4ccccc4c4cc5c6ccccc6n(-c6cccc(-c7ccccc7)c6)c5cc43)cc2)cc1]

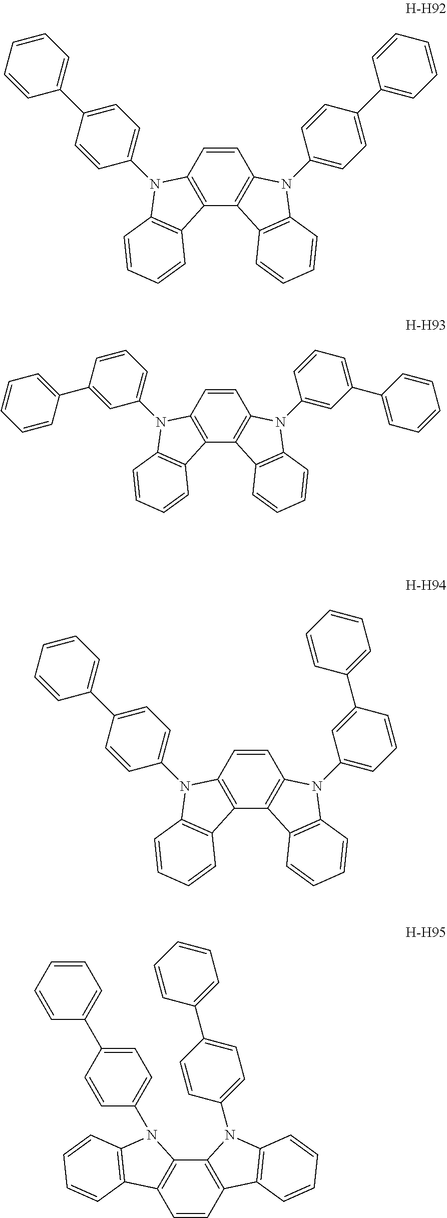 [SMILES: c1ccc(-c2ccc(-n3c4ccccc4c4c5c6ccccc6n(-c6ccc(-c7ccccc7)cc6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4ccc5c6ccccc6n(-c6ccc(-c7ccccc7)cc6)c5c43)cc2)cc1]

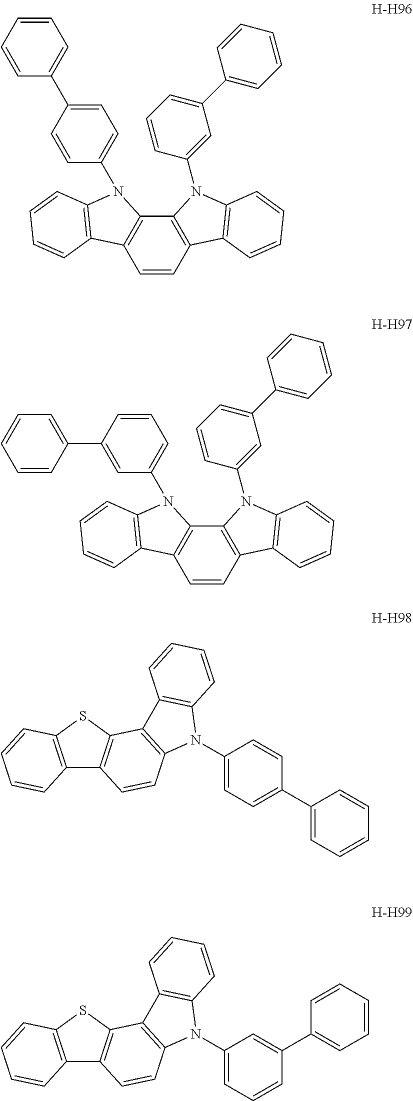 [SMILES: c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-c2ccc(-n3c4ccccc4c4c5sc6ccccc6c5ccc43)cc2)cc1 ; c1ccc(-c2cccc(-n3c4ccccc4c4c5sc6ccccc6c5ccc43)c2)cc1]

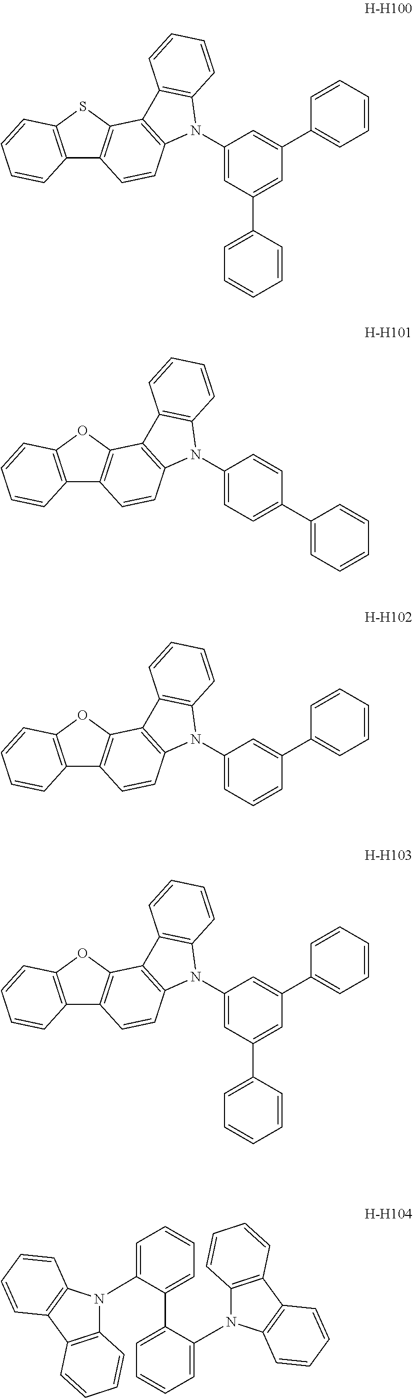 [SMILES: c1ccc(-c2cc(-c3ccccc3)cc(-n3c4ccccc4c4c5sc6ccccc6c5ccc43)c2)cc1 ; c1ccc(-c2ccc(-n3c4ccccc4c4c5oc6ccccc6c5ccc43)cc2)cc1 ; c1ccc(-c2cccc(-n3c4ccccc4c4c5oc6ccccc6c5ccc43)c2)cc1 ; c1ccc(-c2cc(-c3ccccc3)cc(-n3c4ccccc4c4c5oc6ccccc6c5ccc43)c2)cc1 ; c1ccc(-n2c3ccccc3c3ccccc32)c(-c2ccccc2-n2c3ccccc3c3ccccc32)c1]

In one or more embodiments, the bipolar host may be a compound from group HEH1, but embodiments of the present disclosure are not limited thereto:

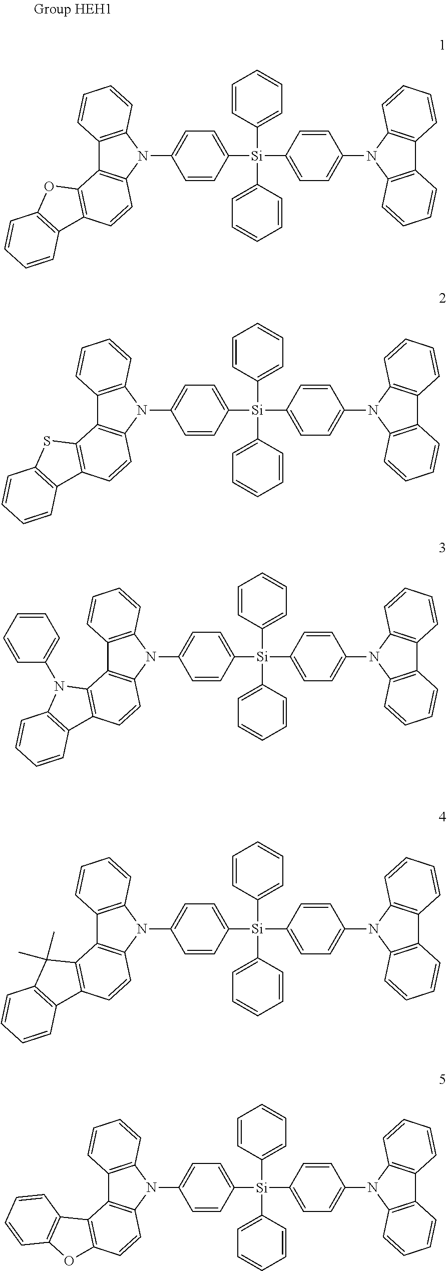

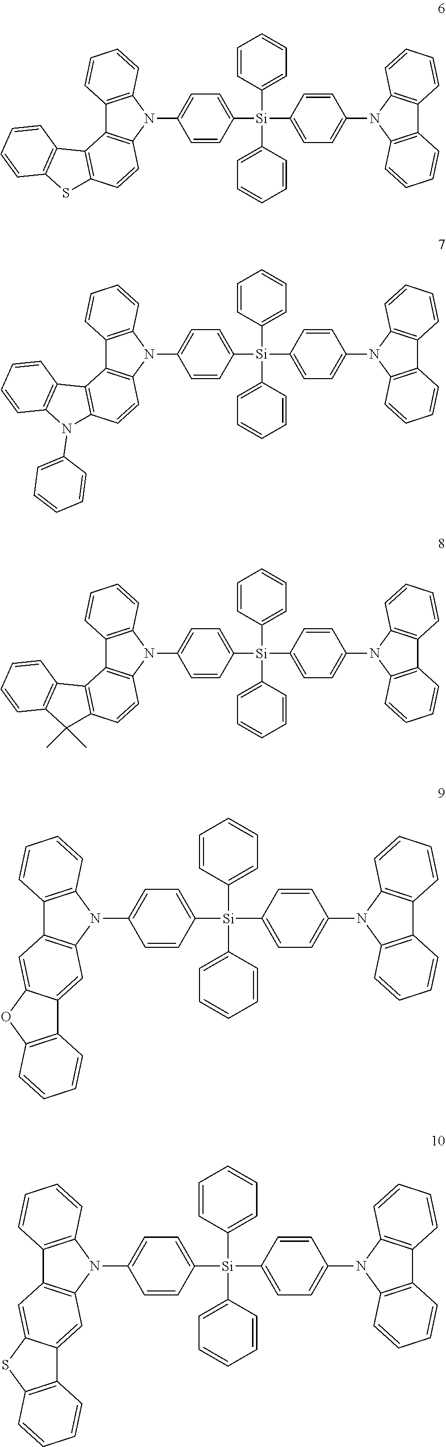

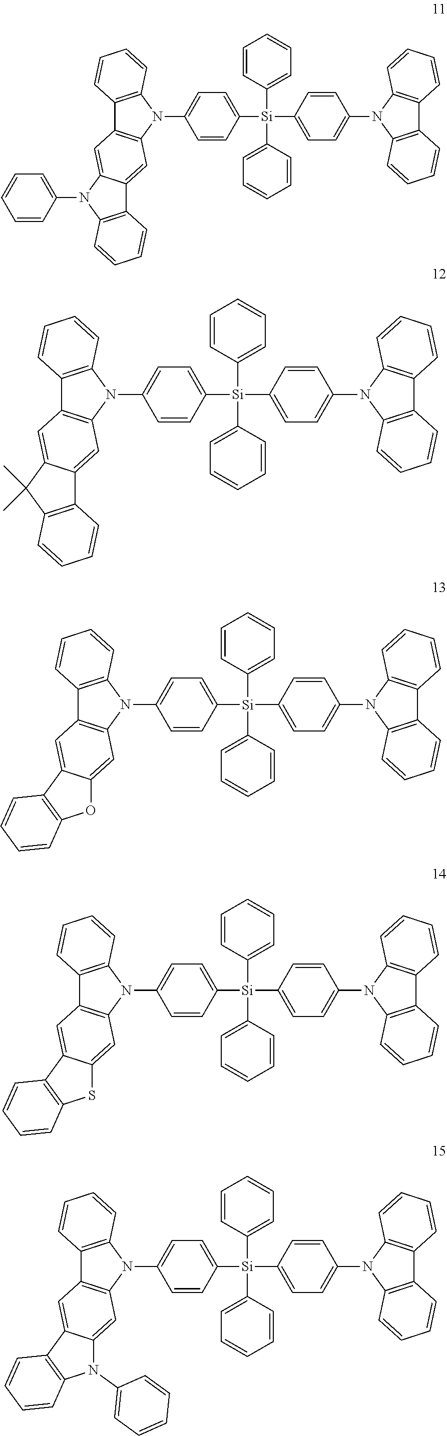

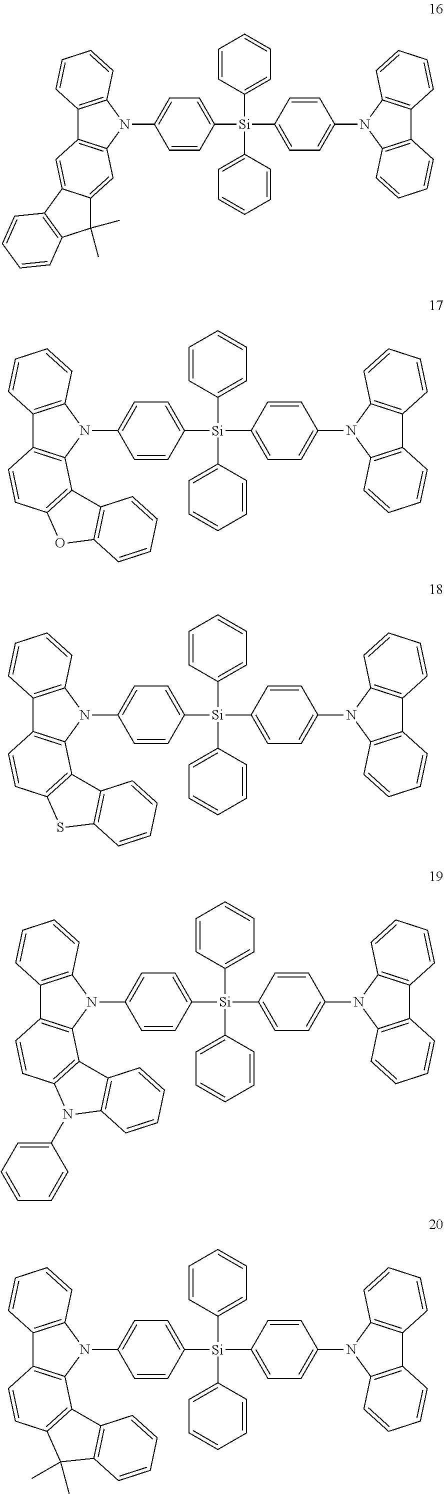

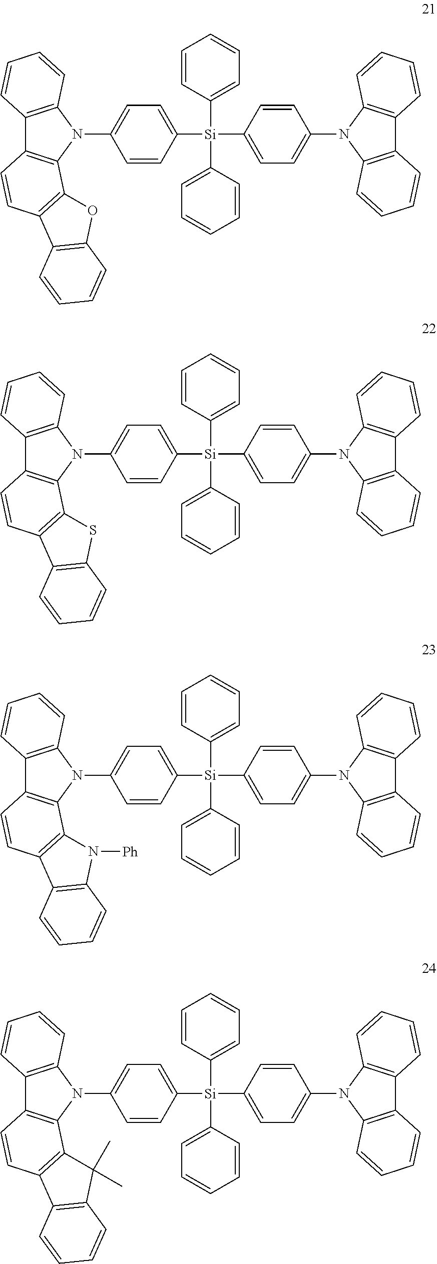

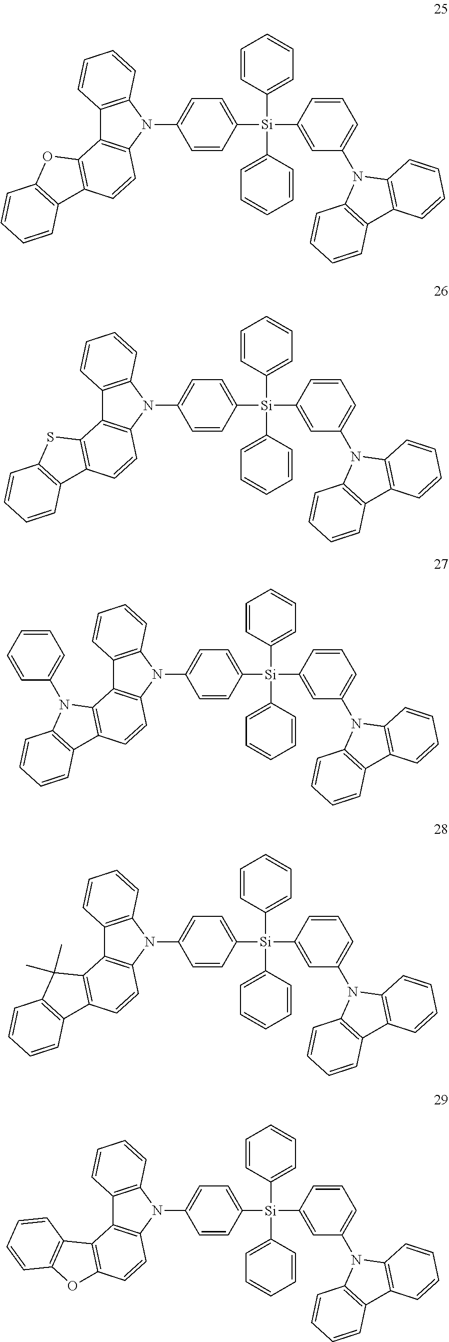

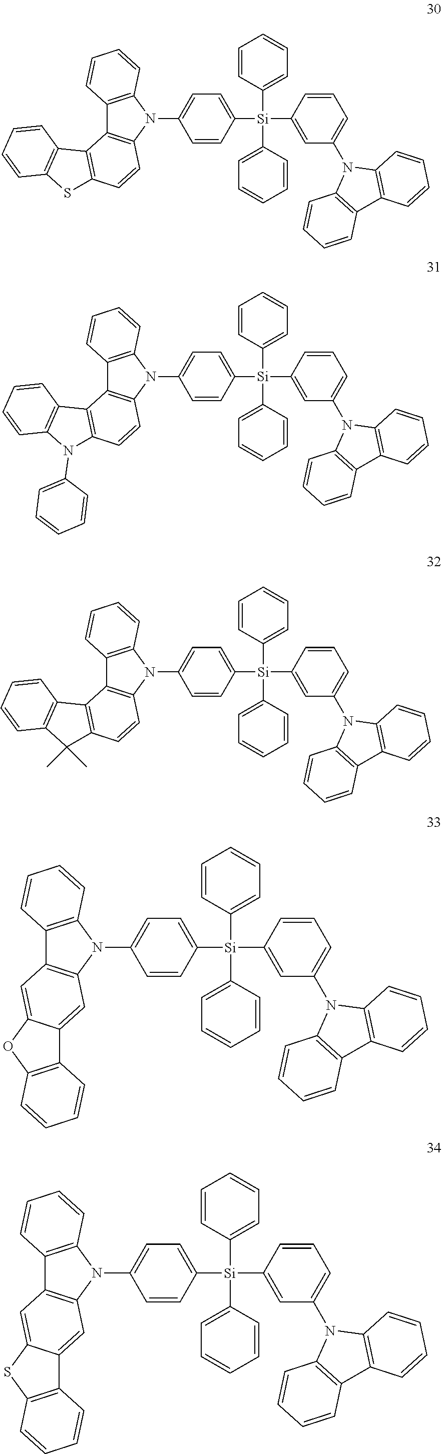

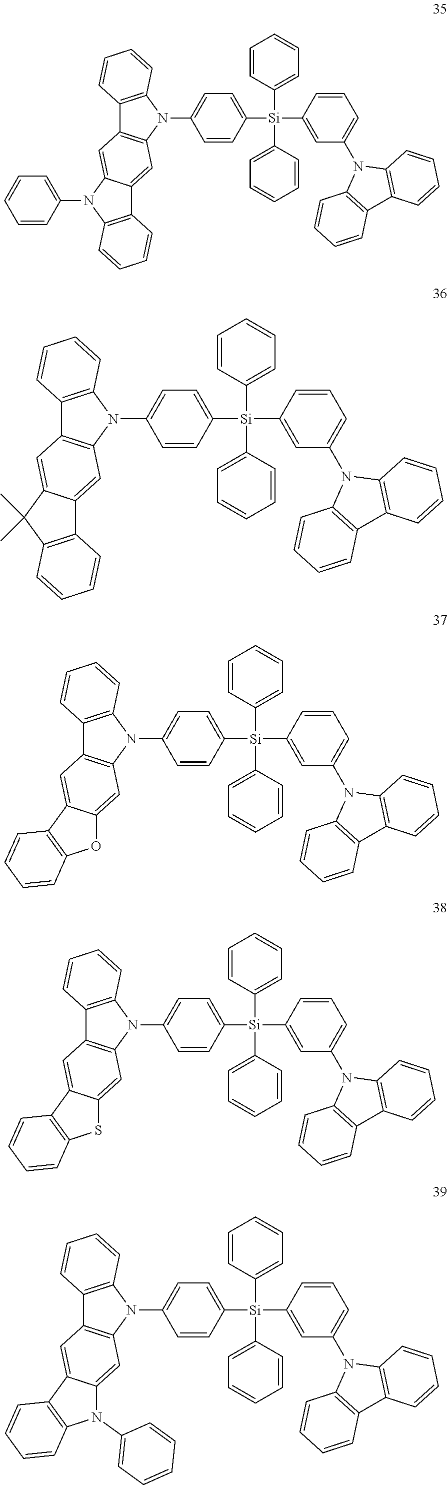

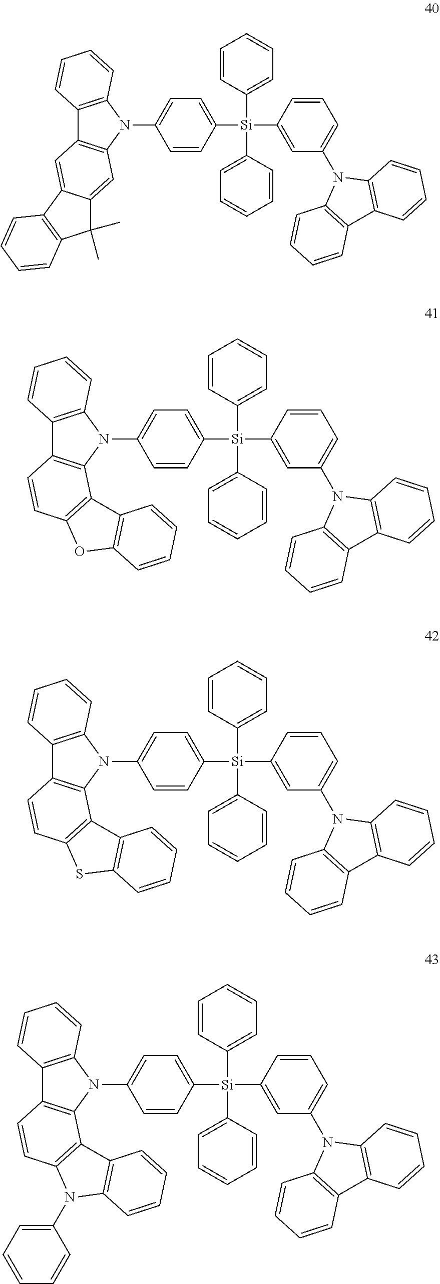

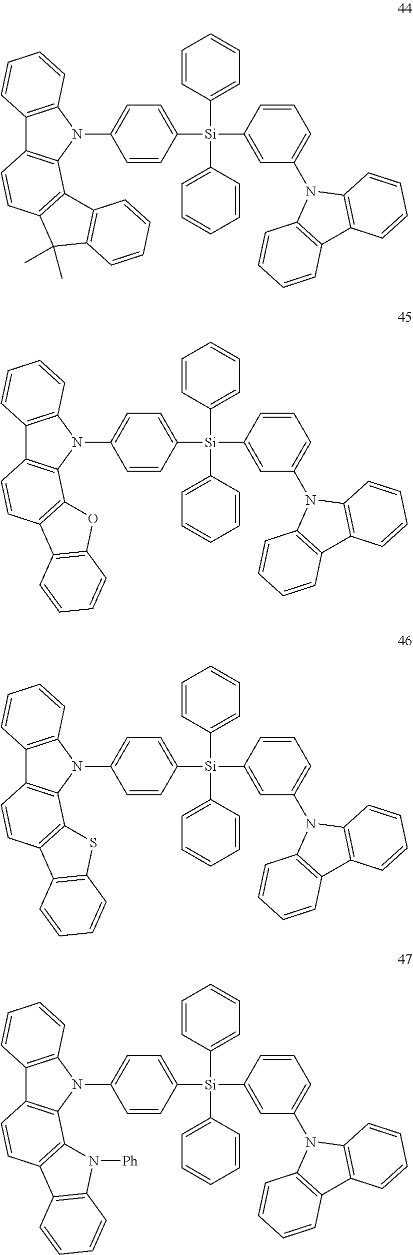

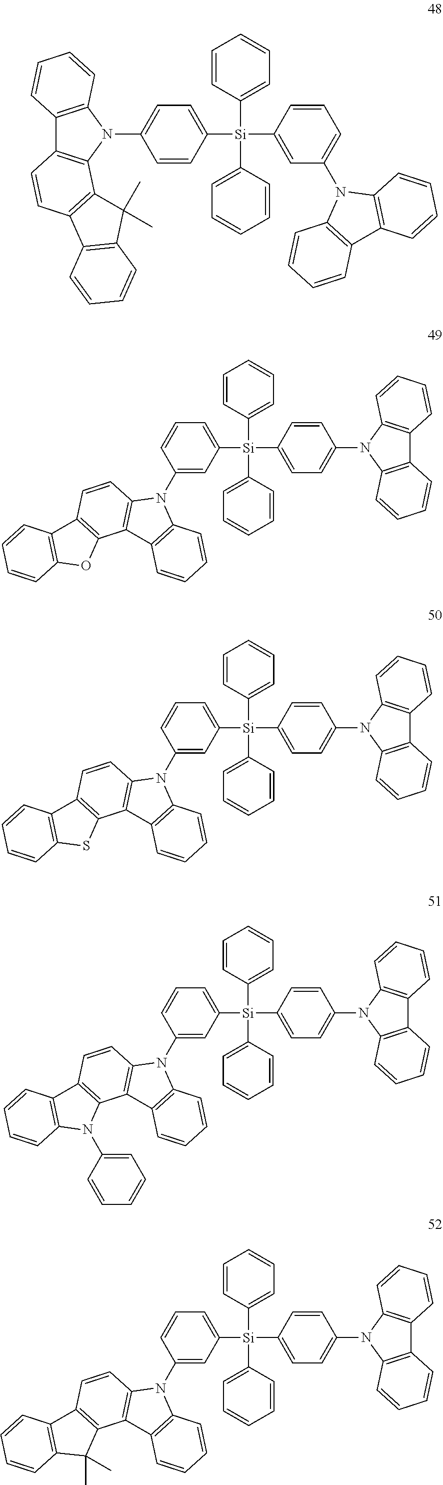

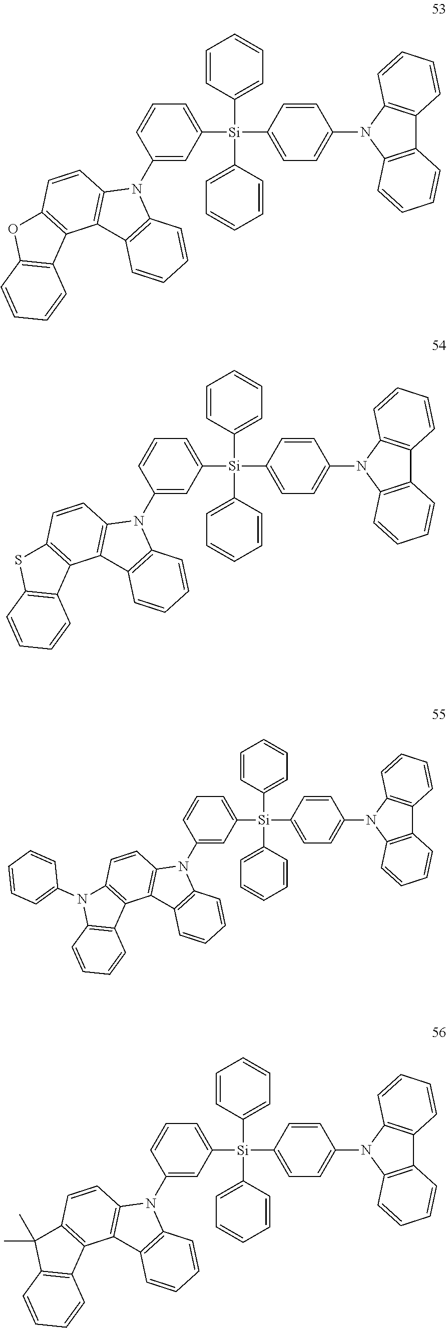

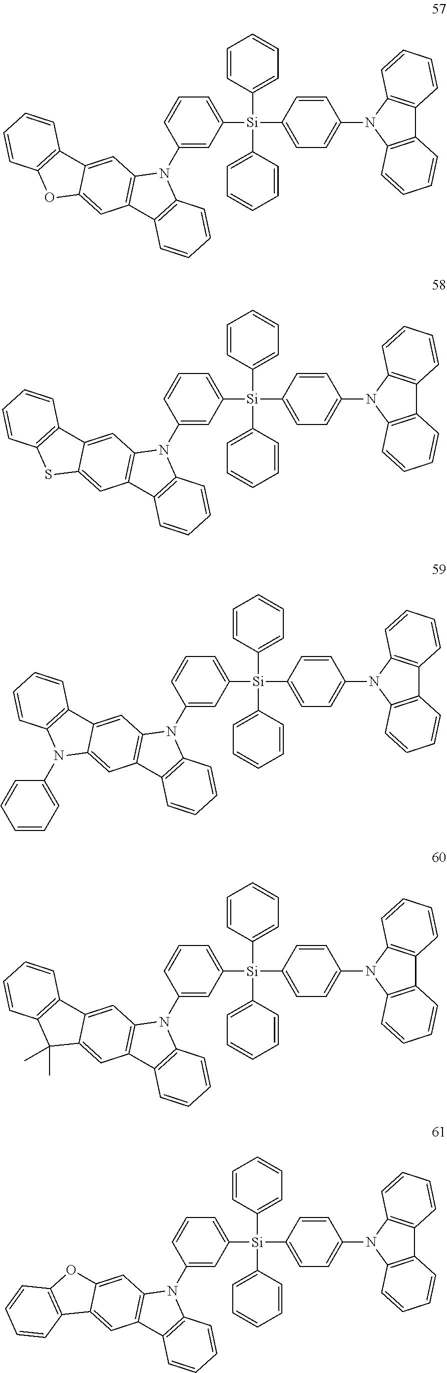

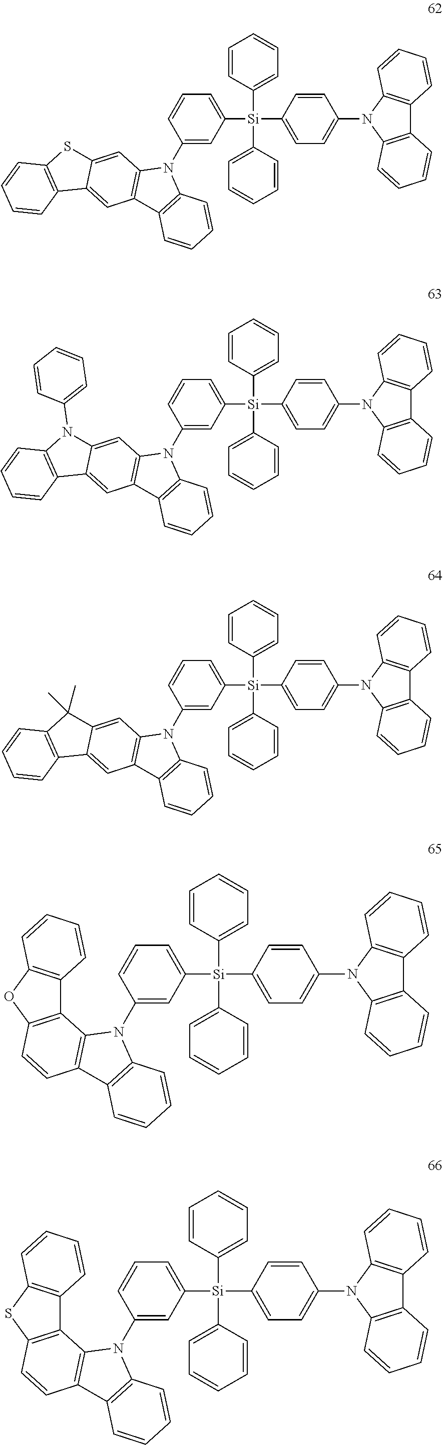

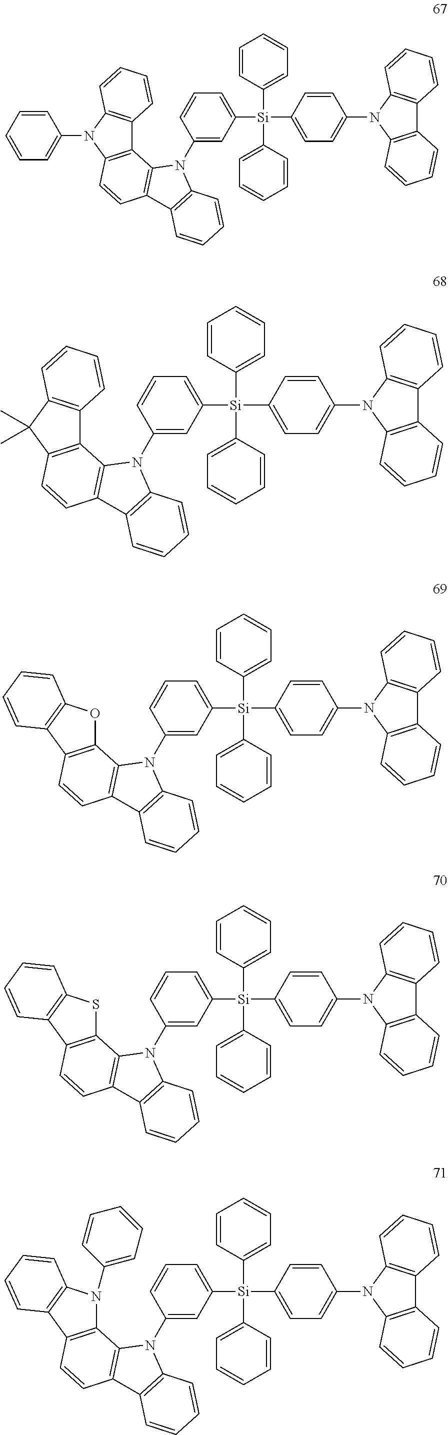

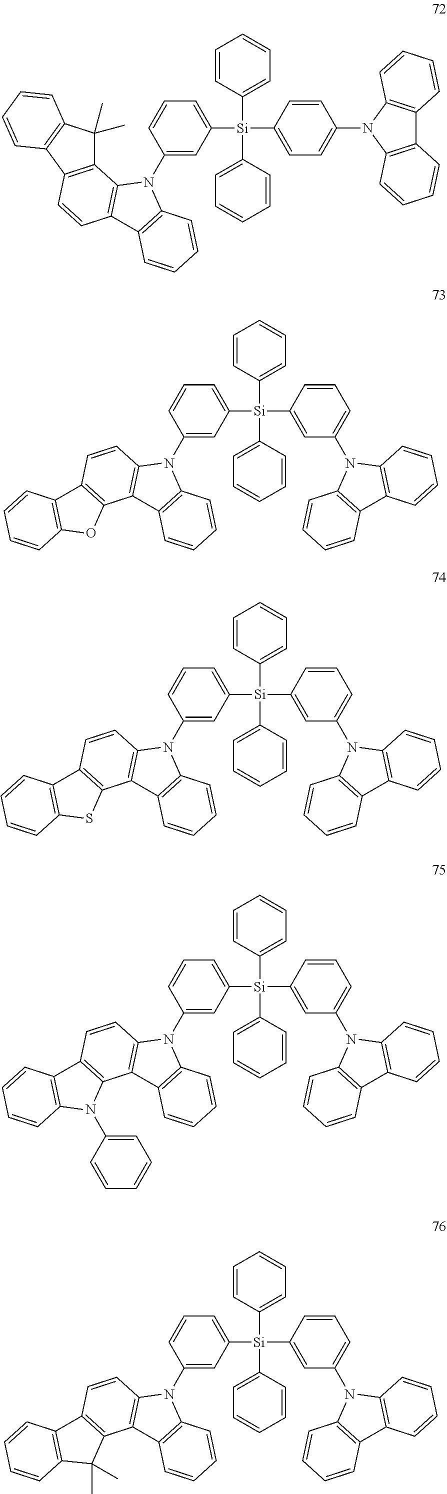

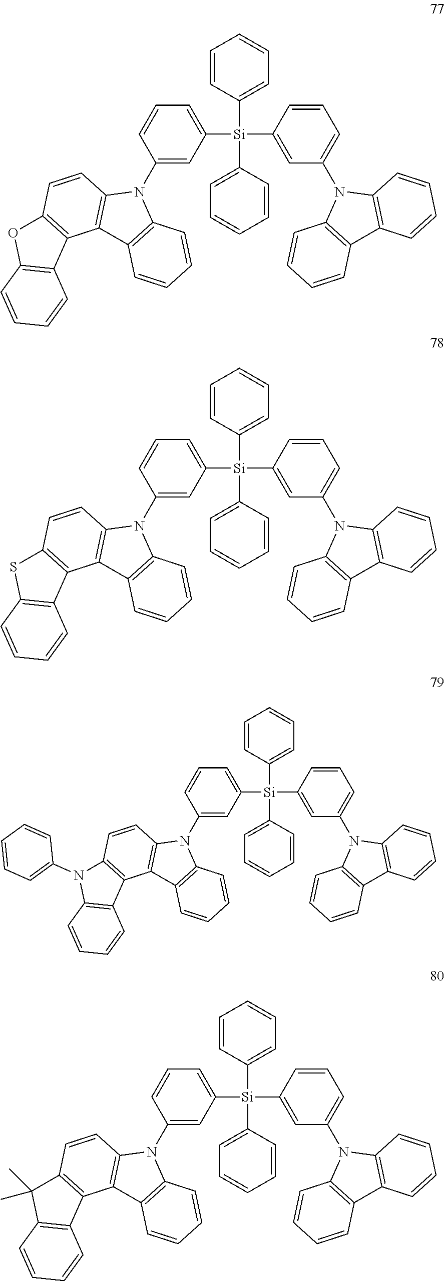

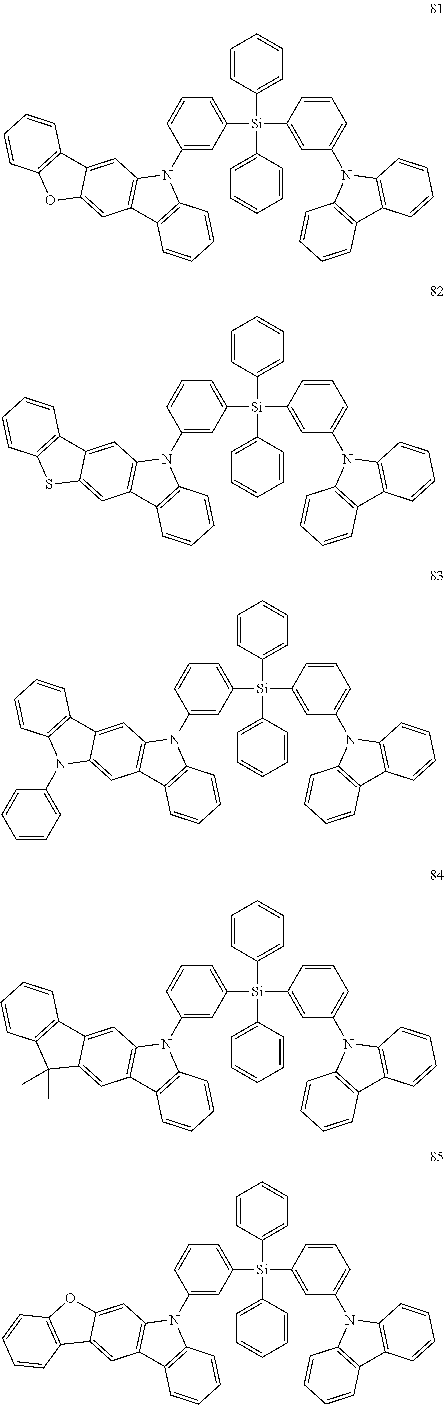

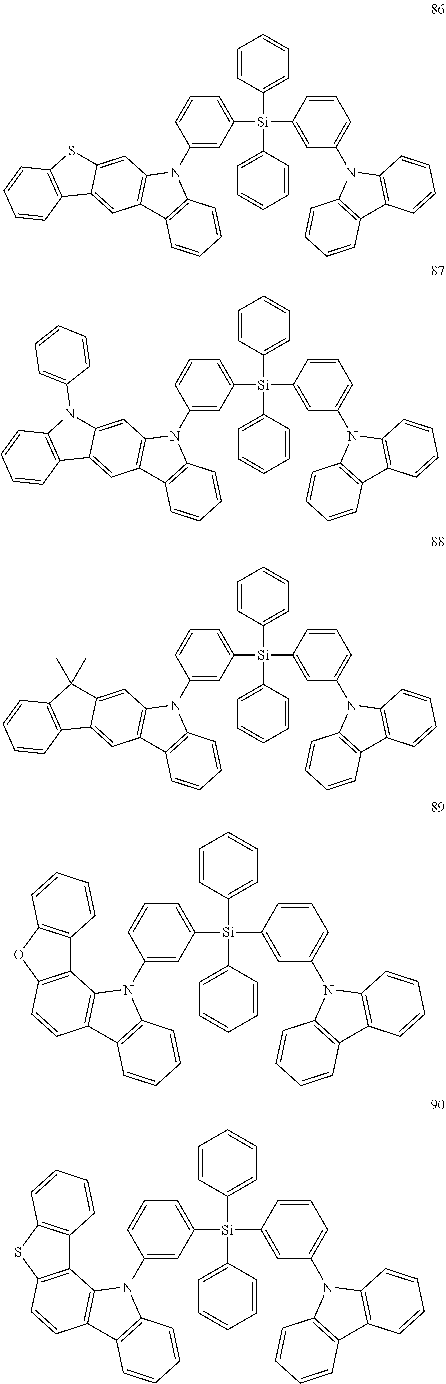

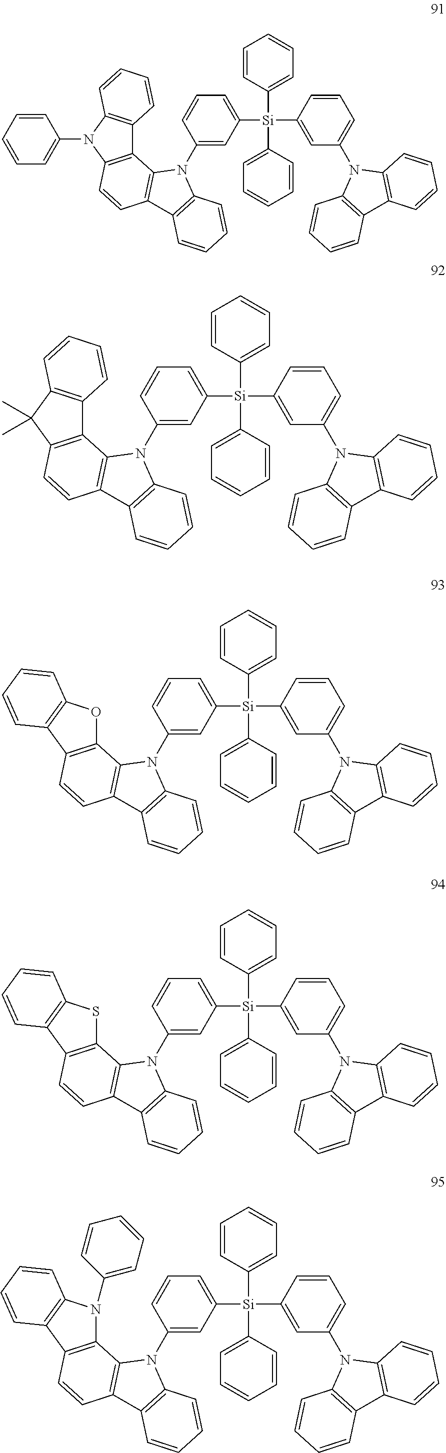

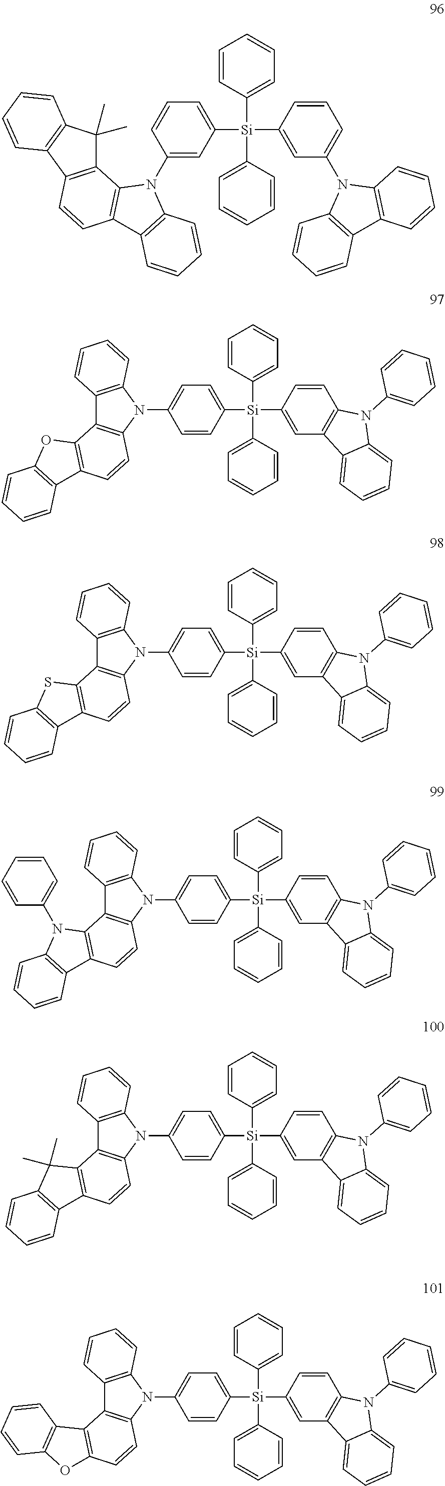

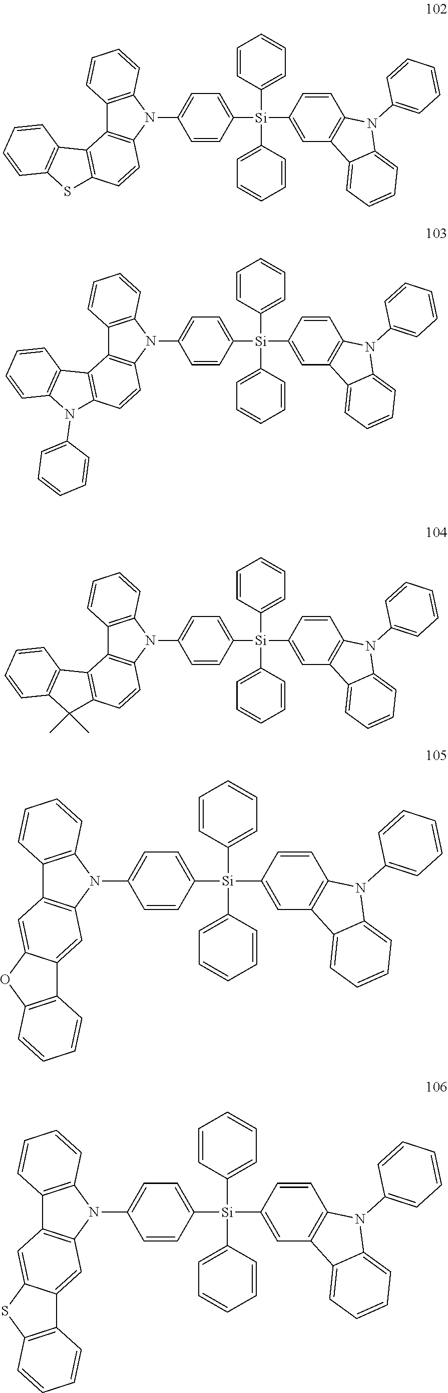

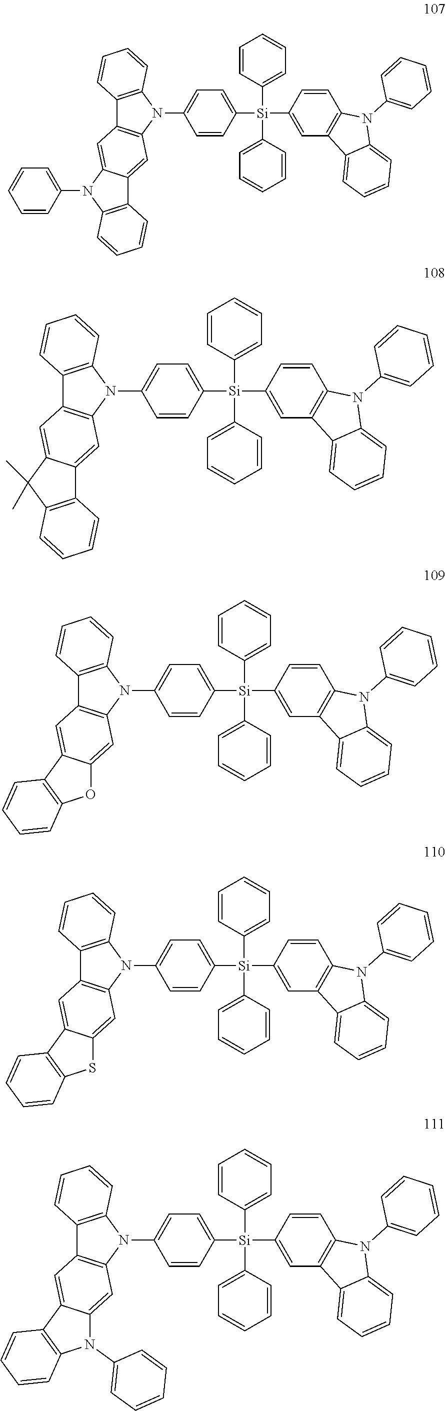

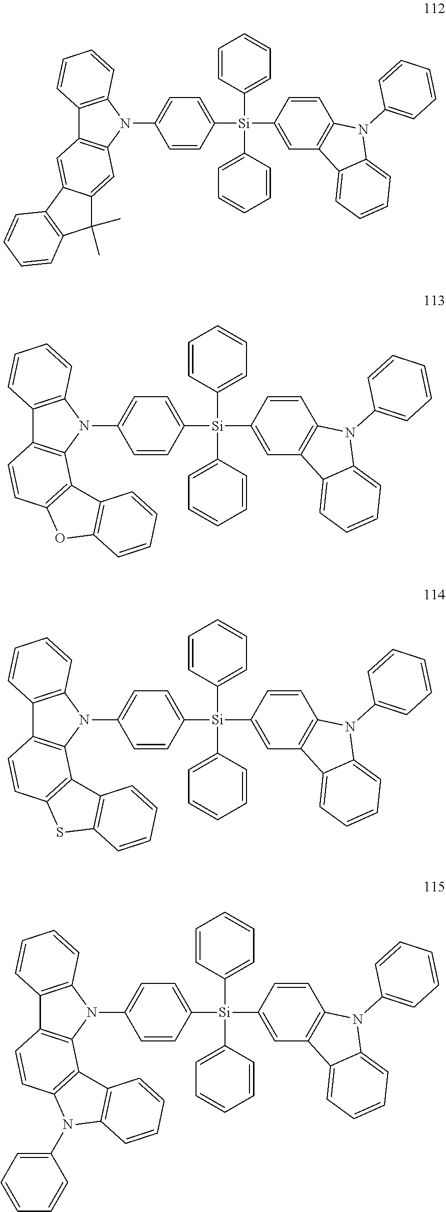

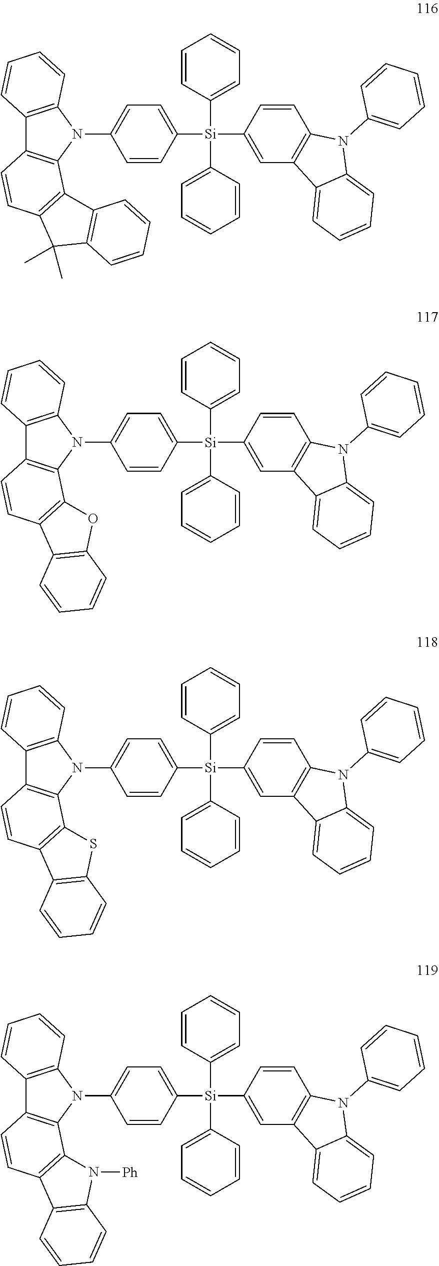

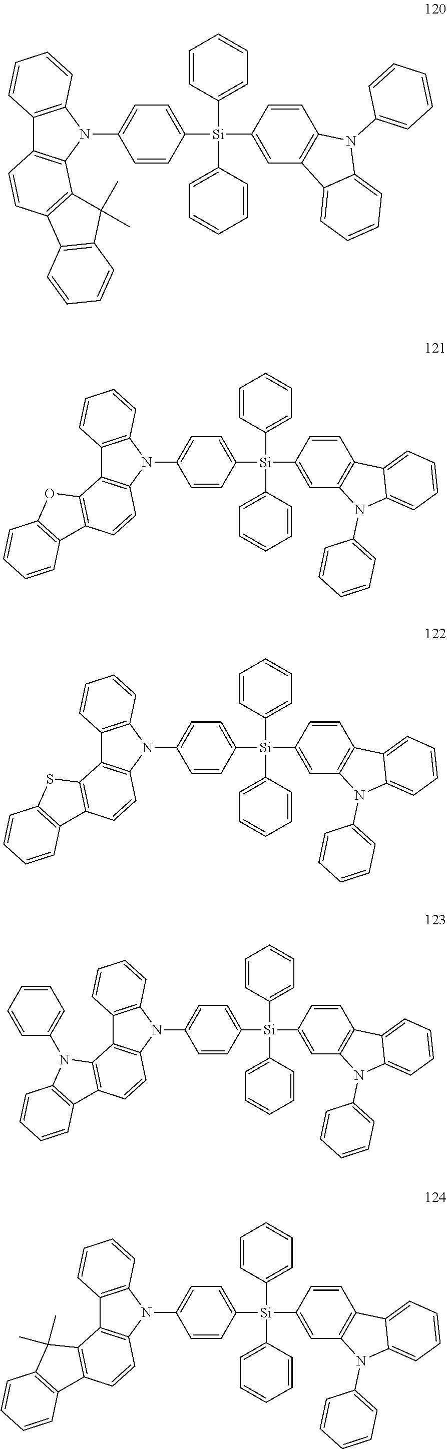

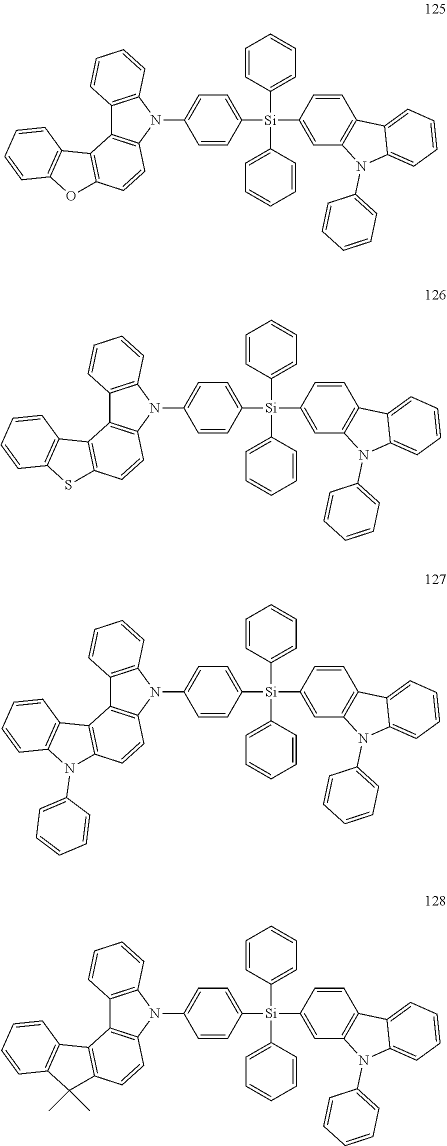

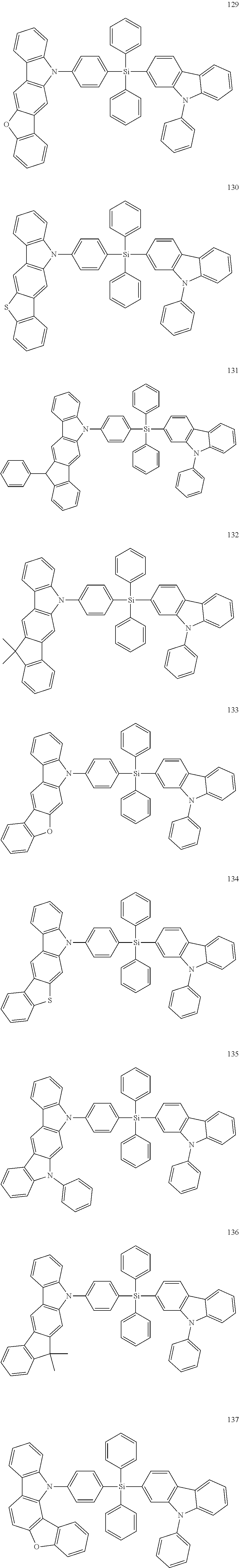

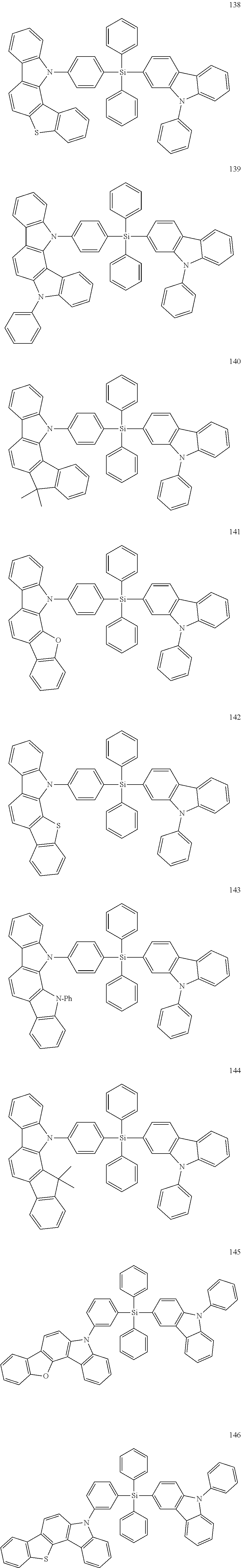

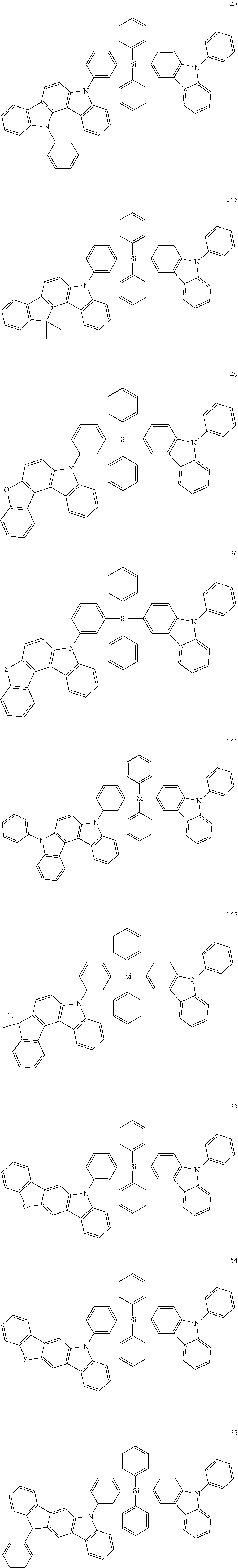

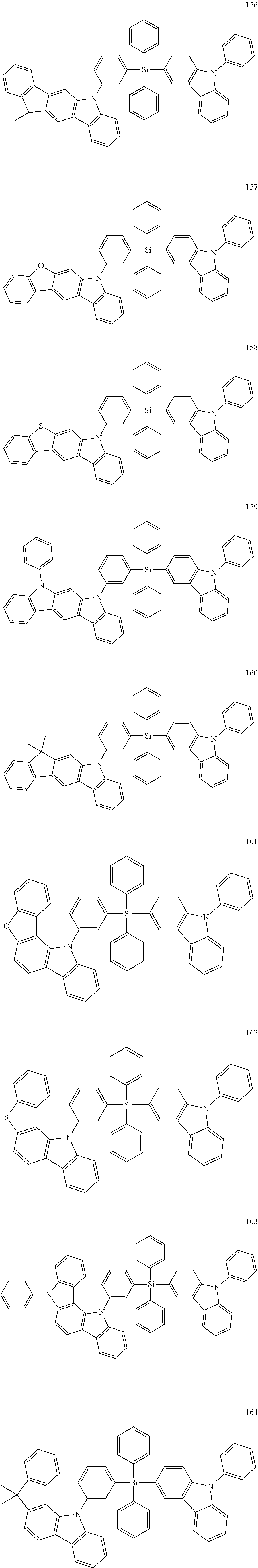

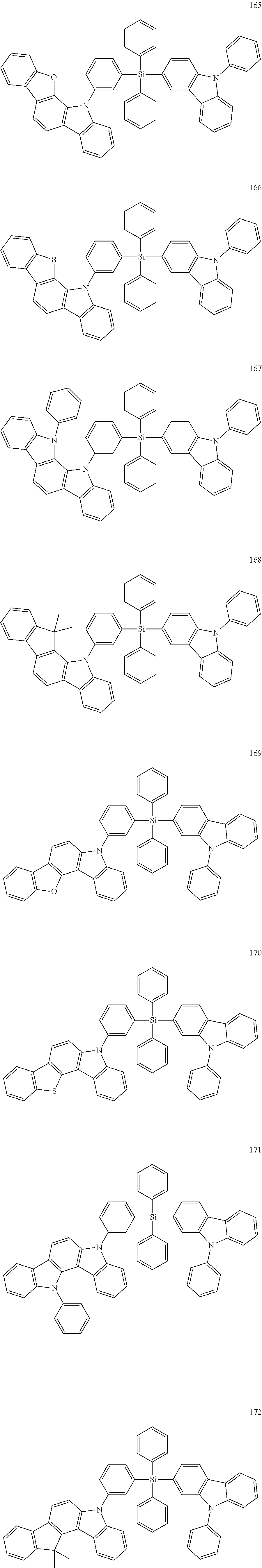

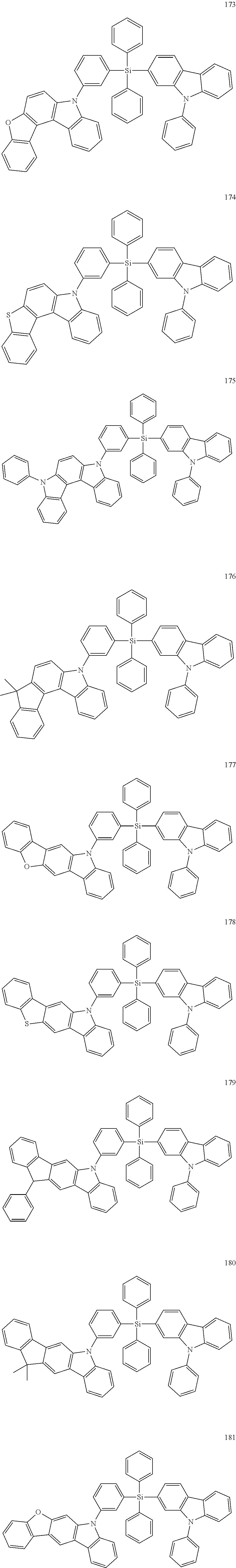

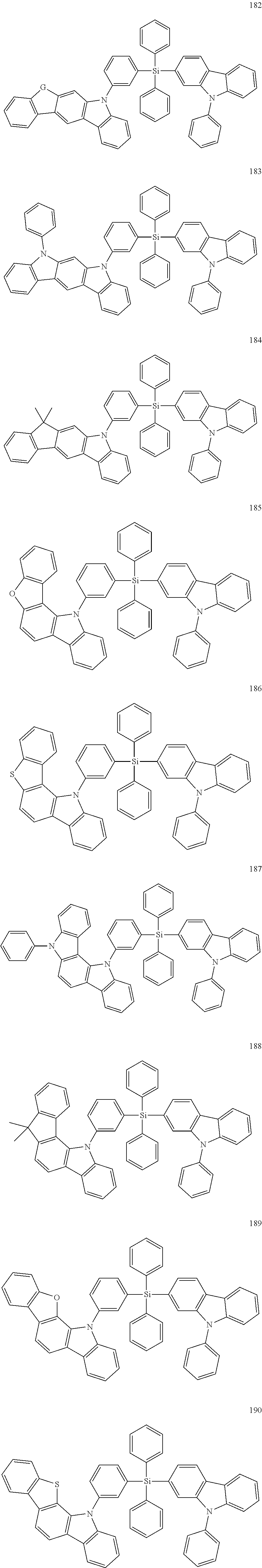

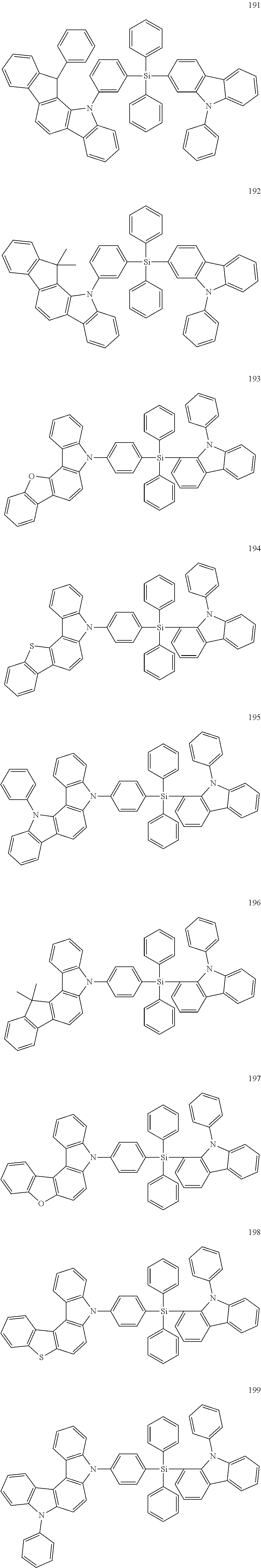

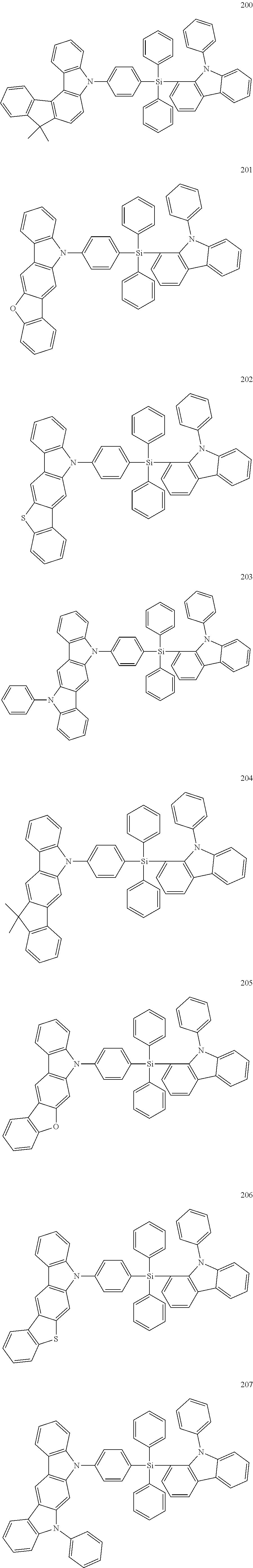

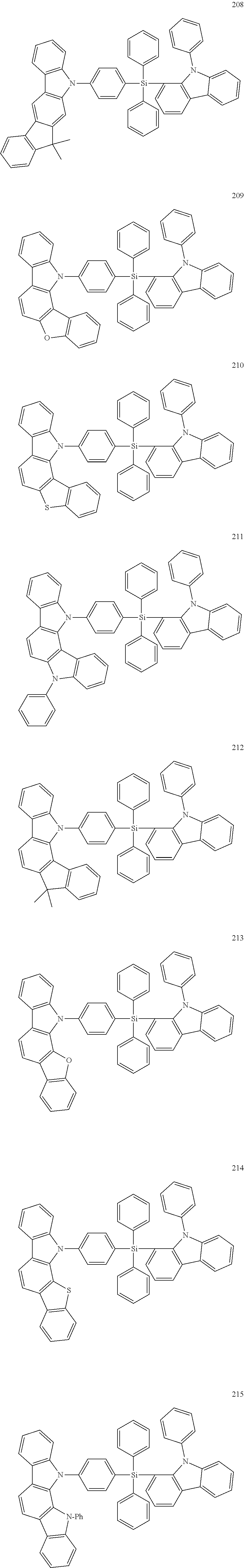

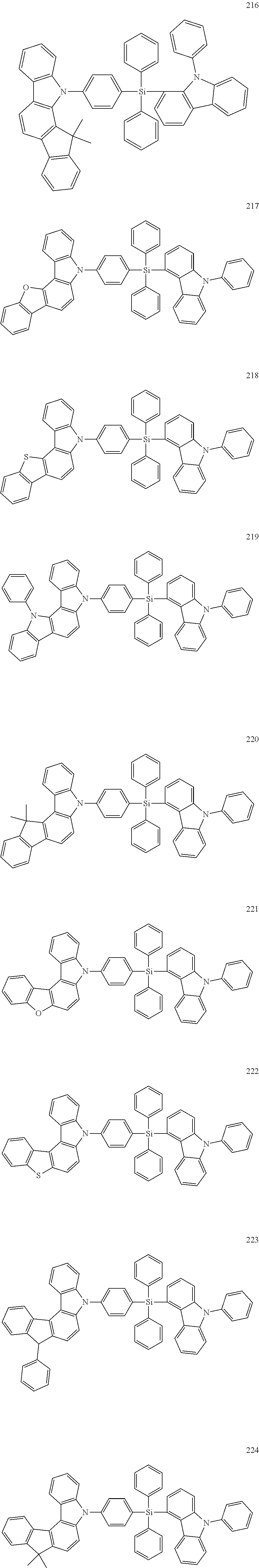

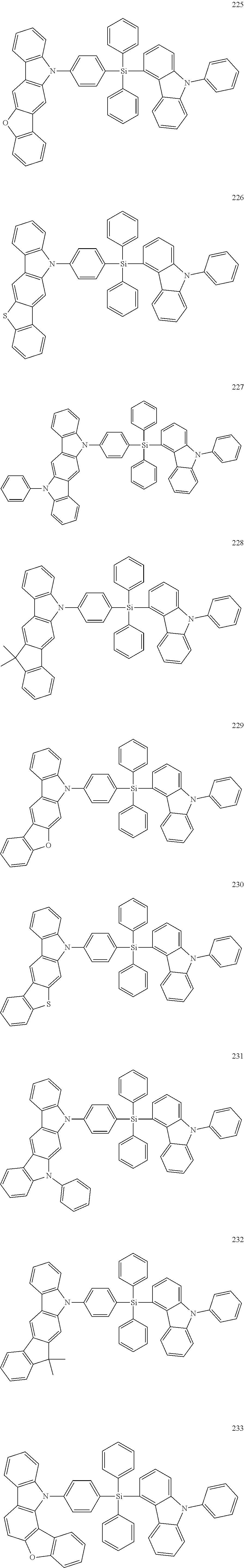

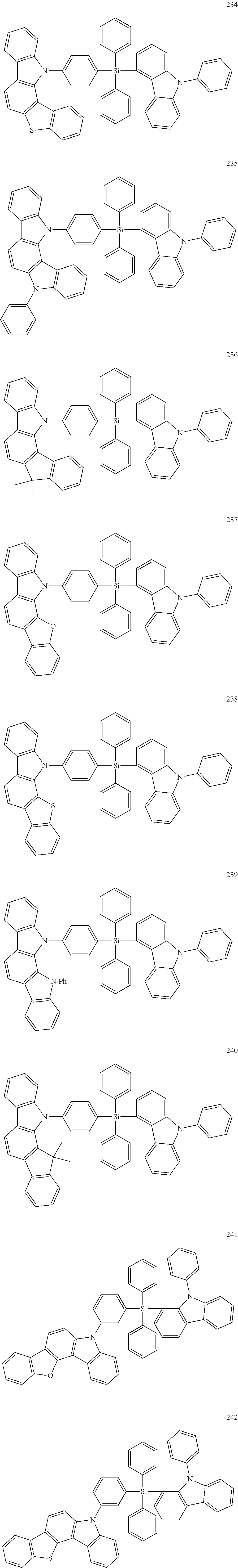

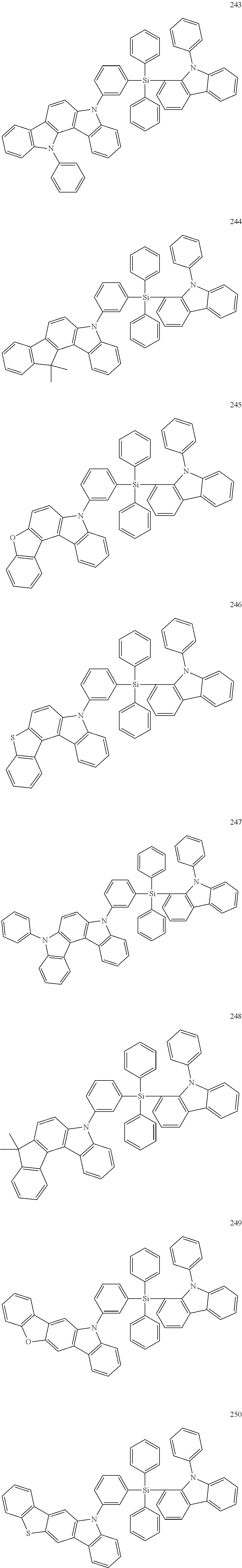

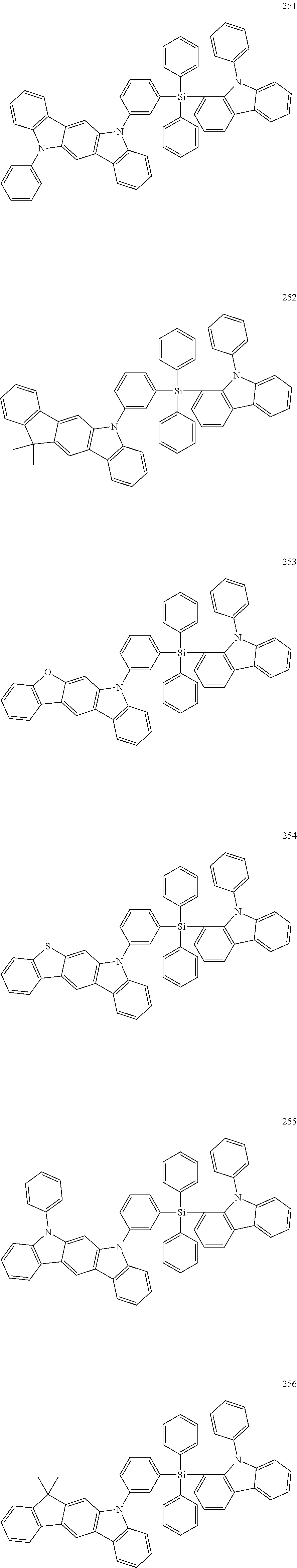

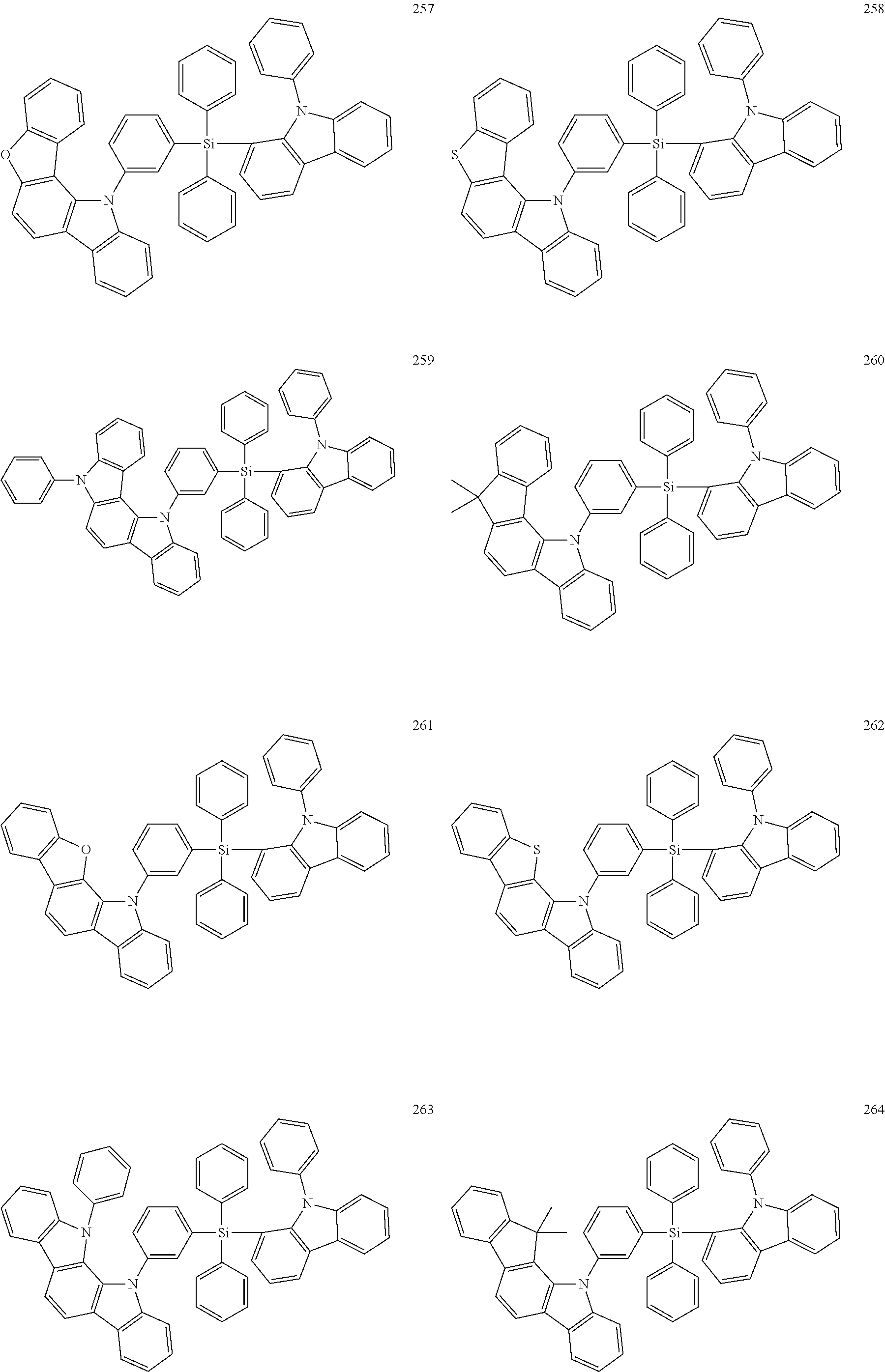

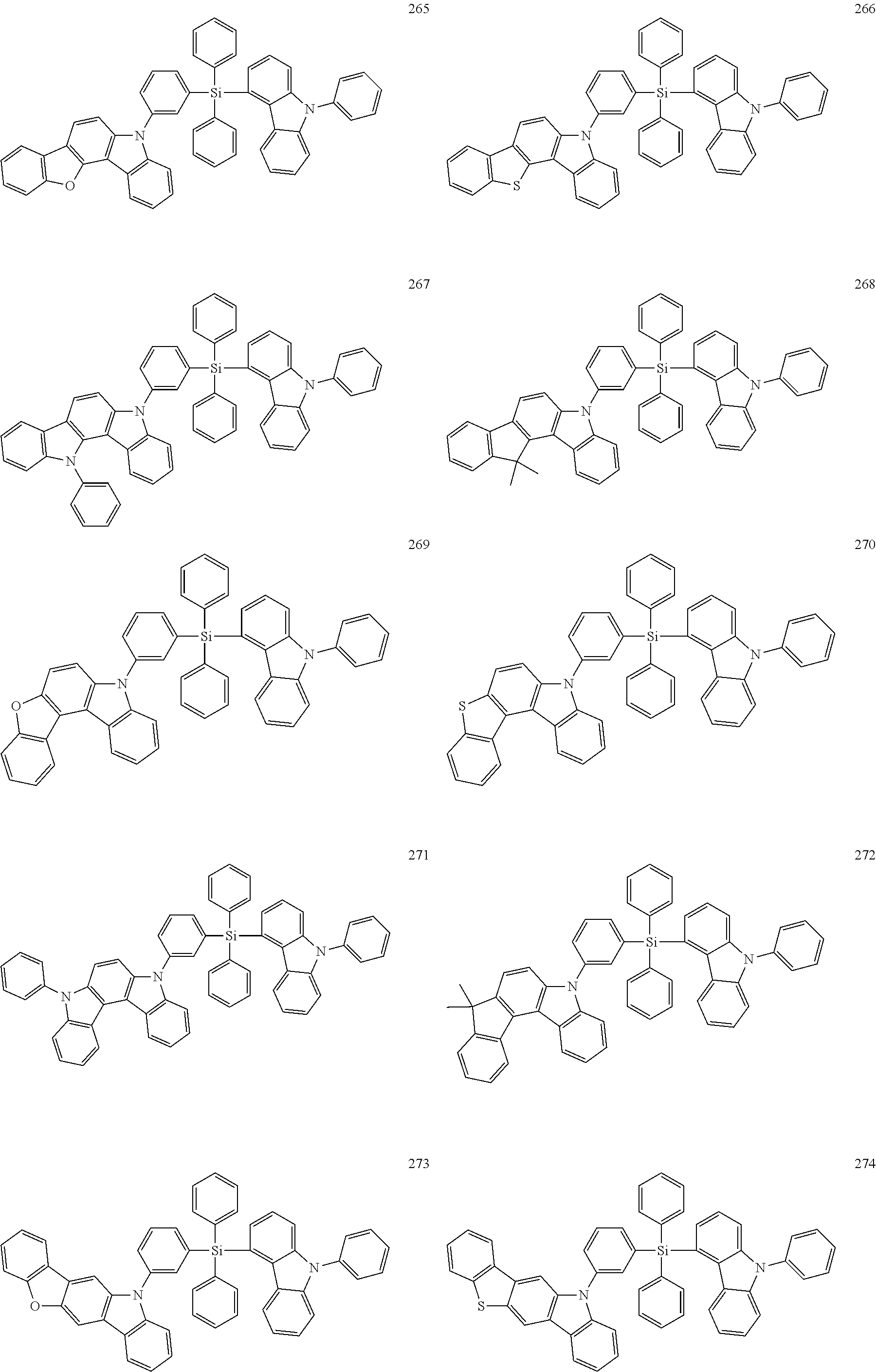

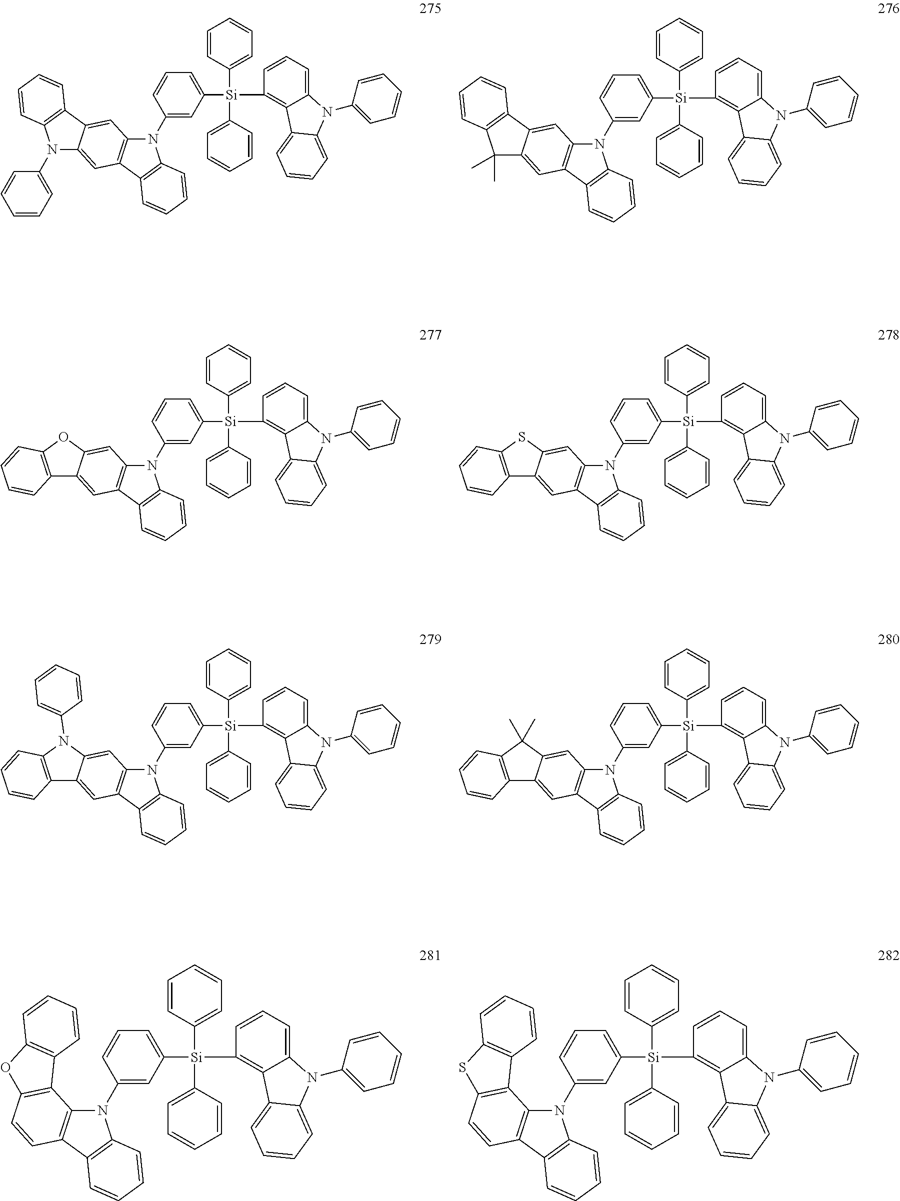

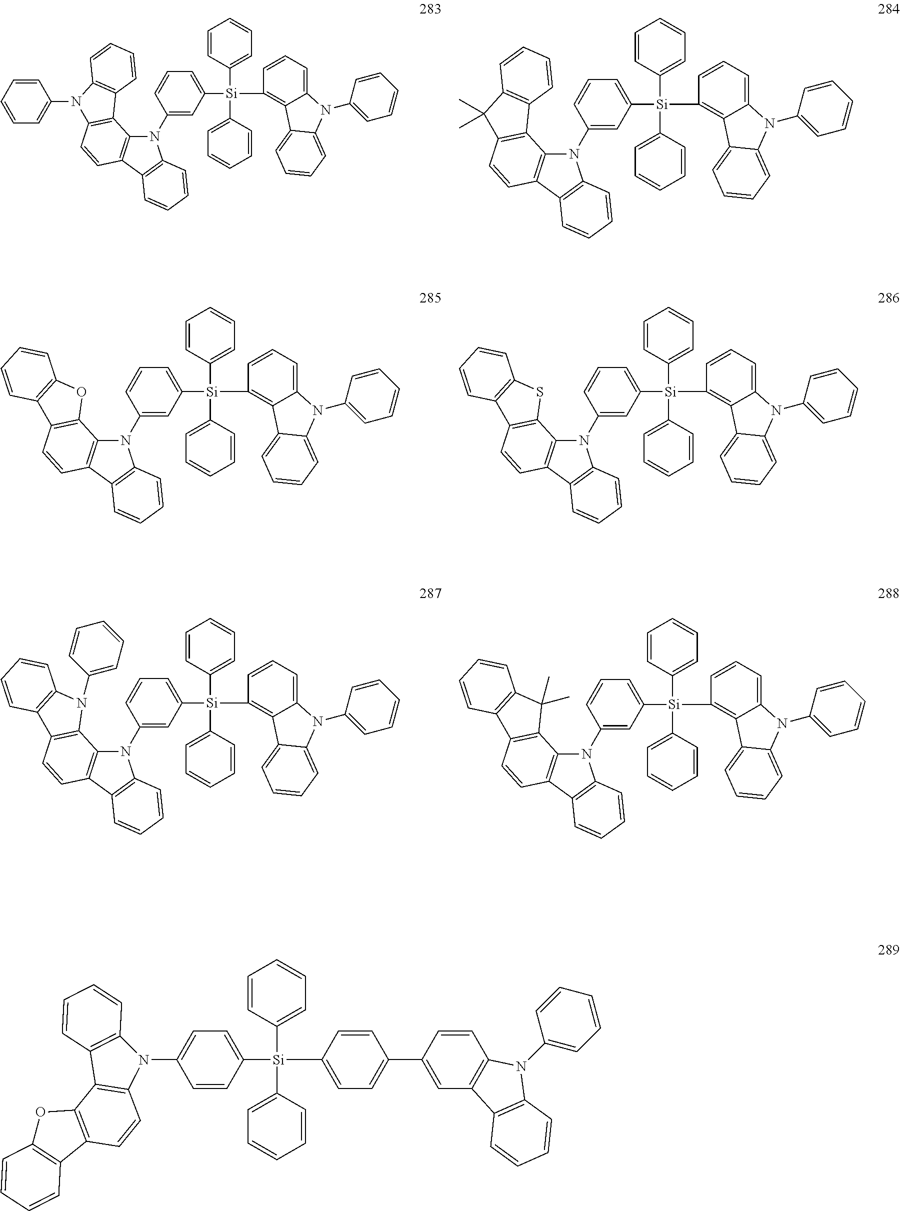

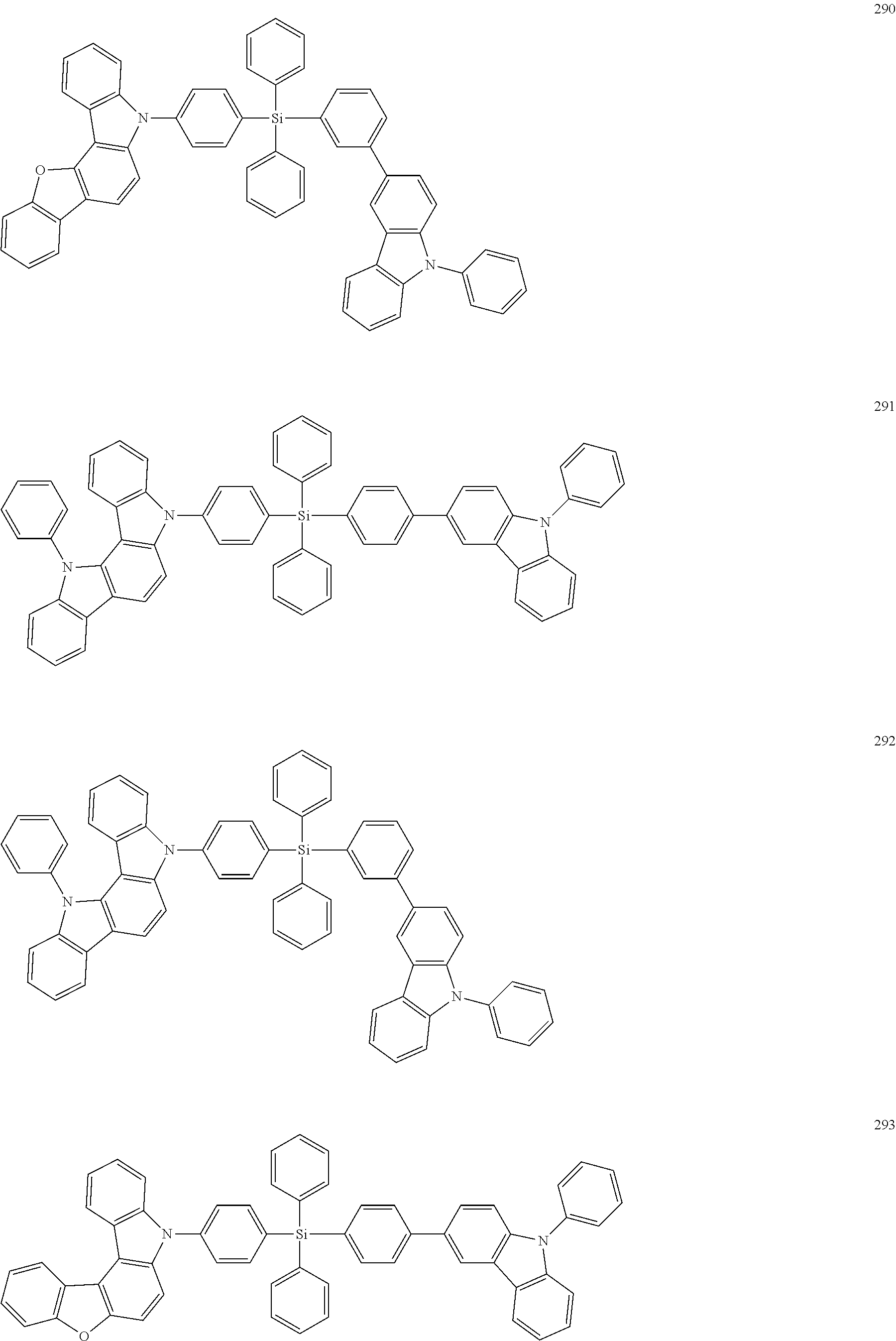

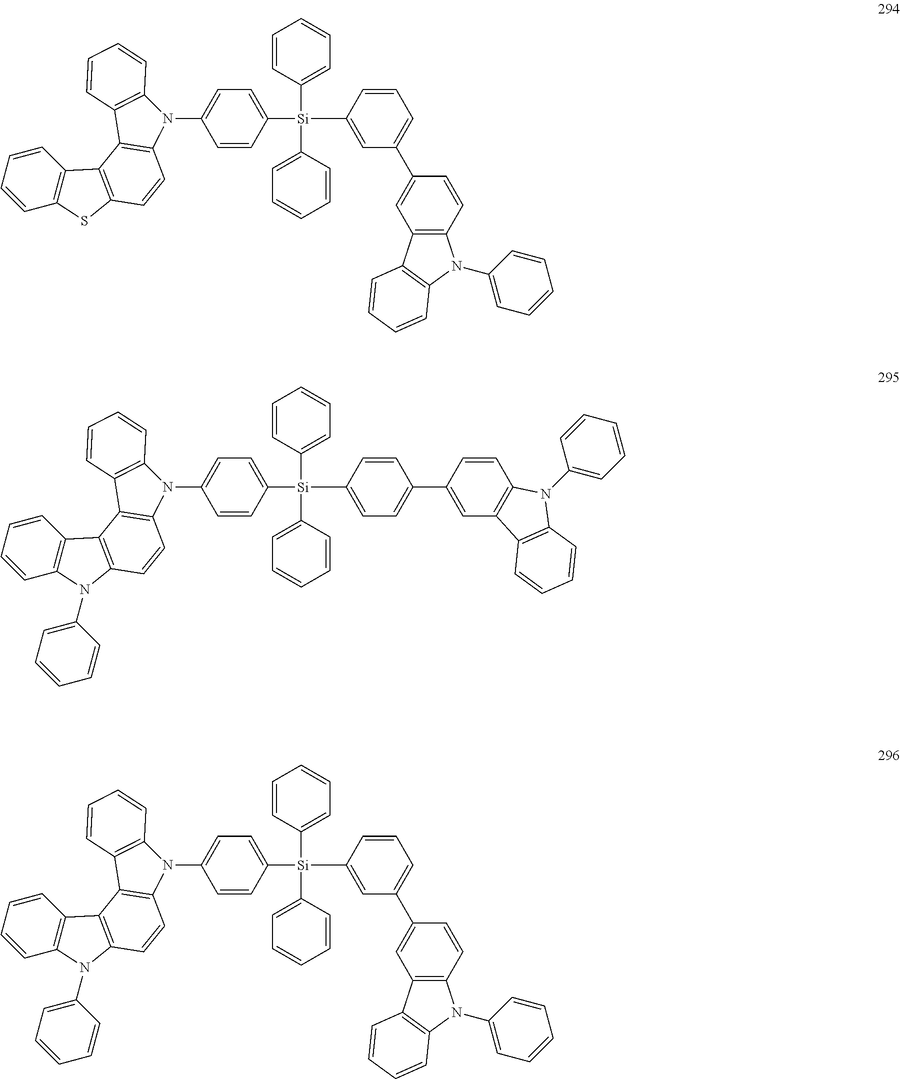

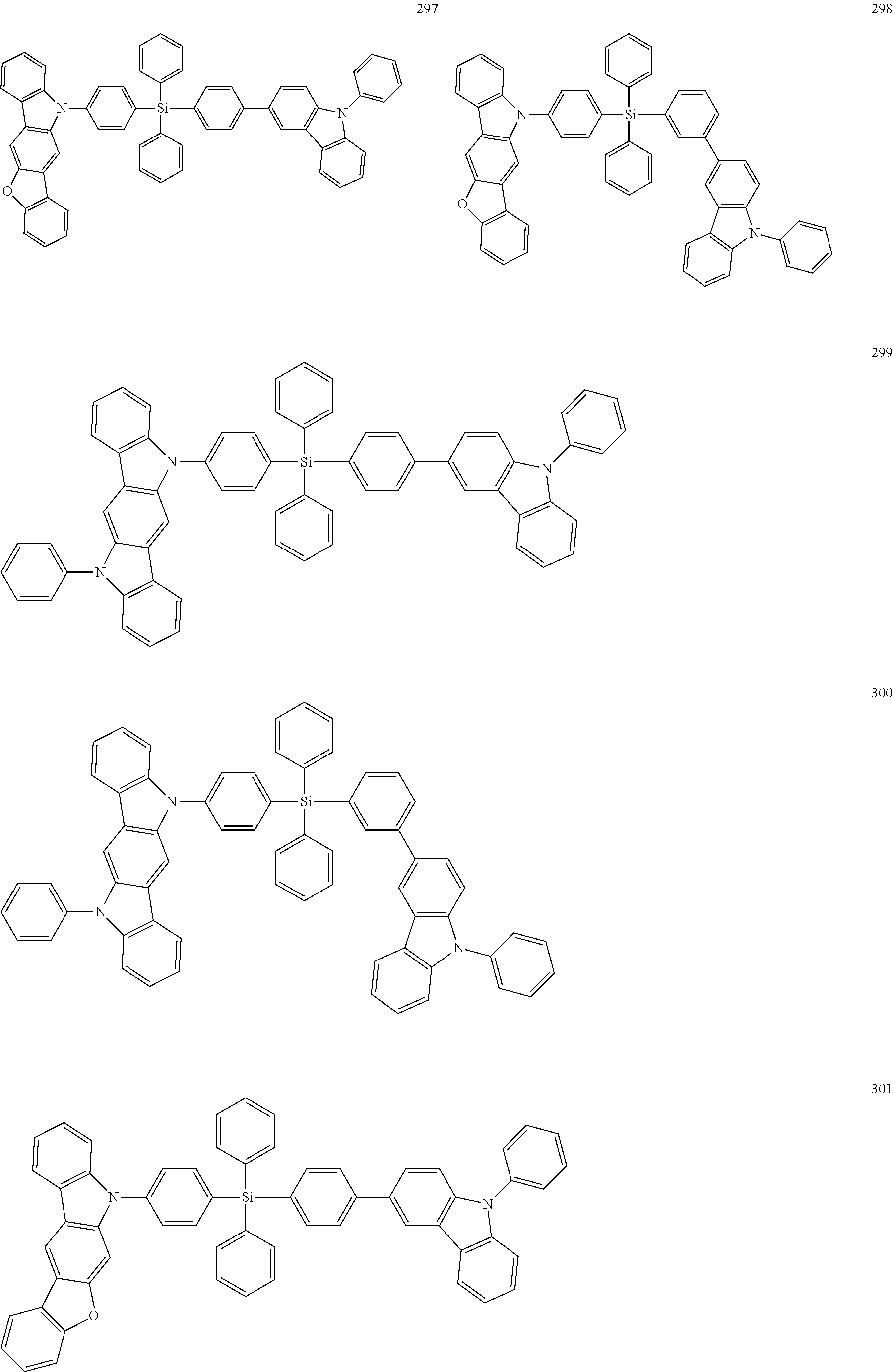

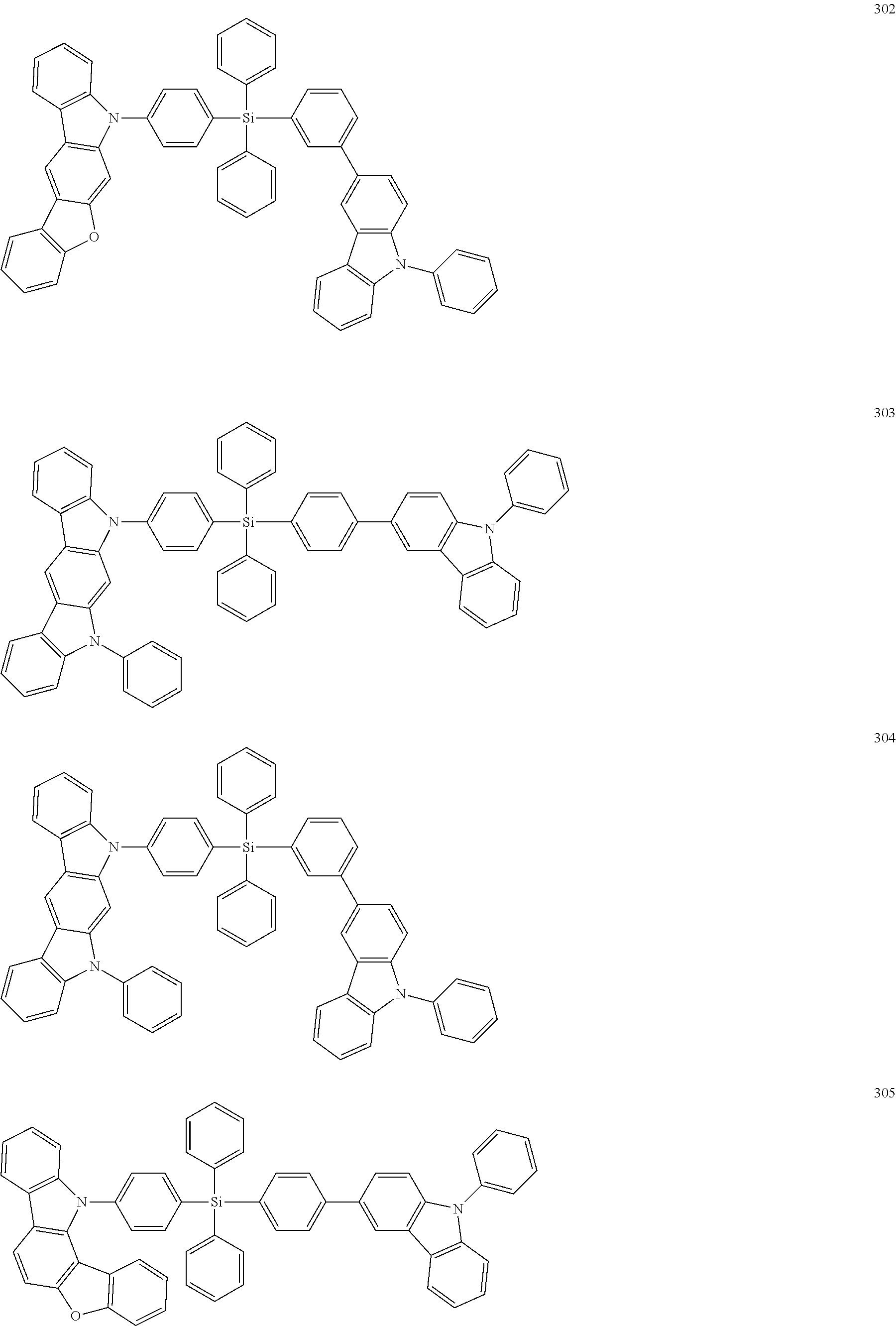

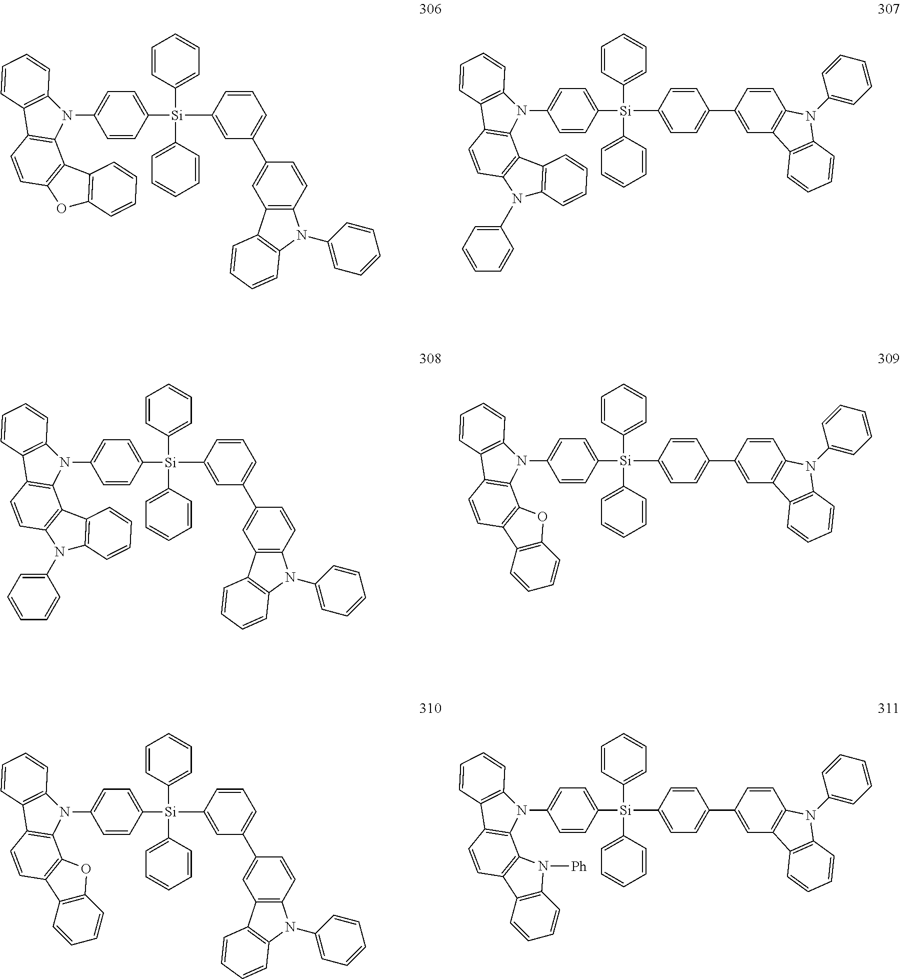

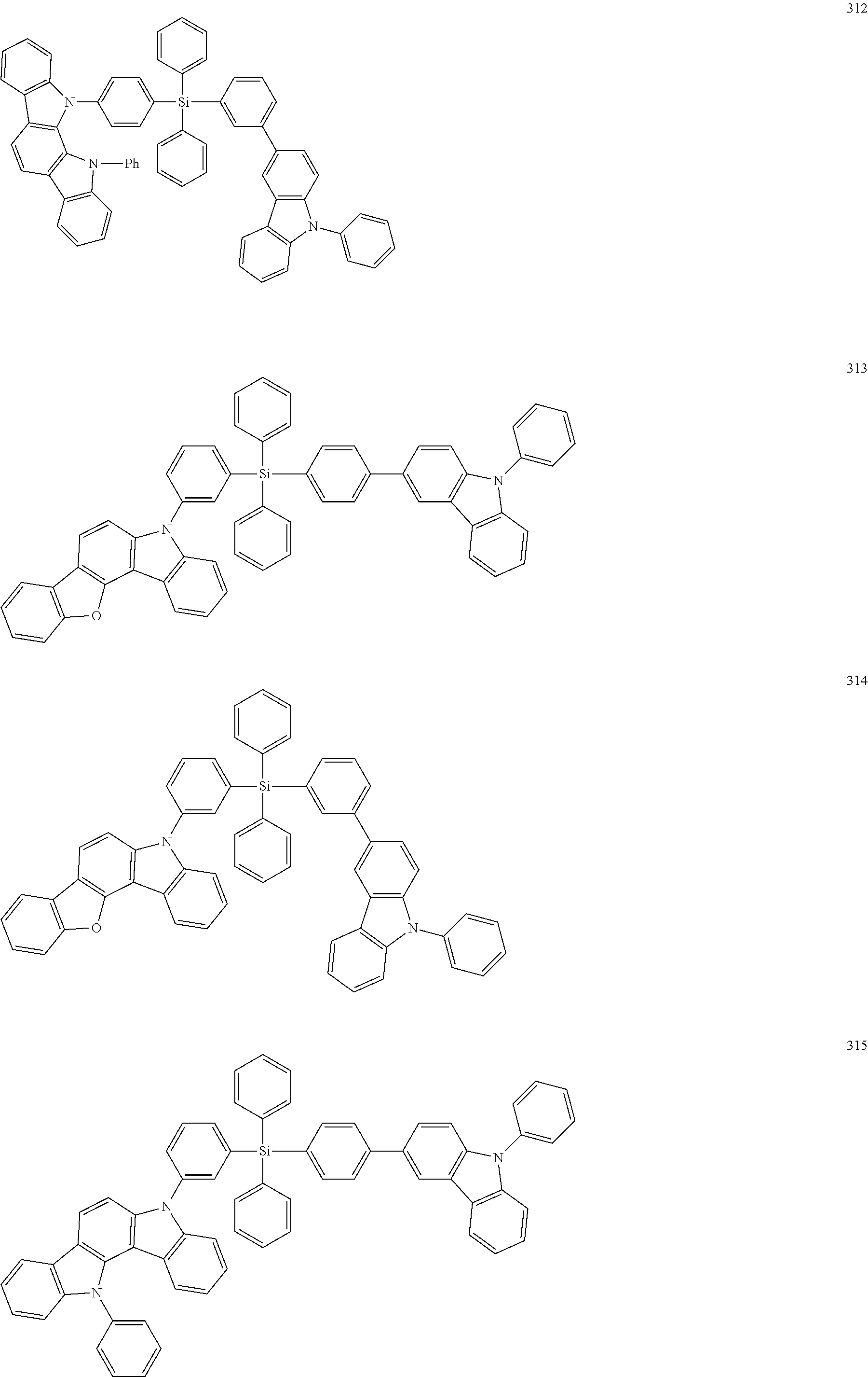

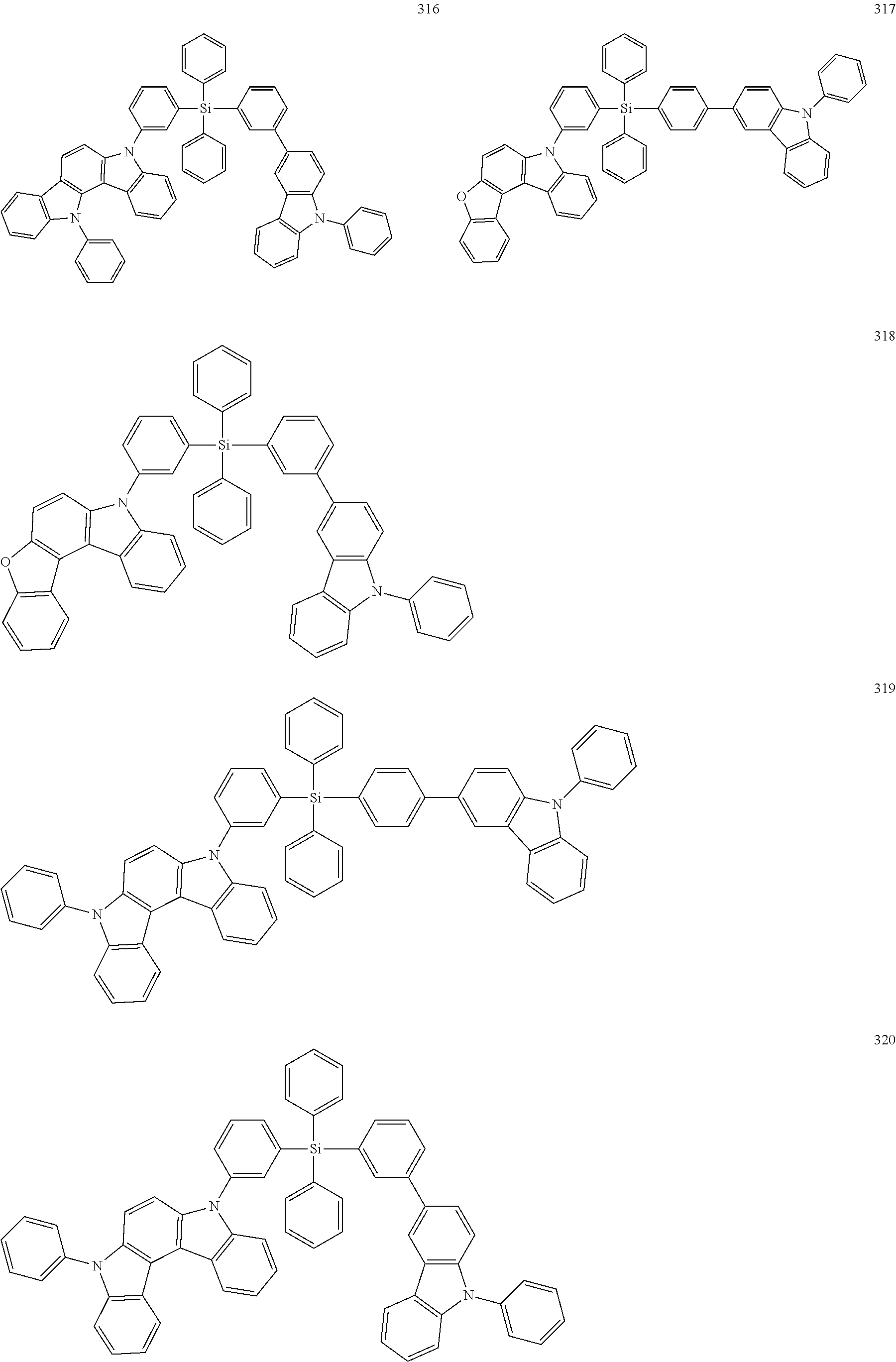

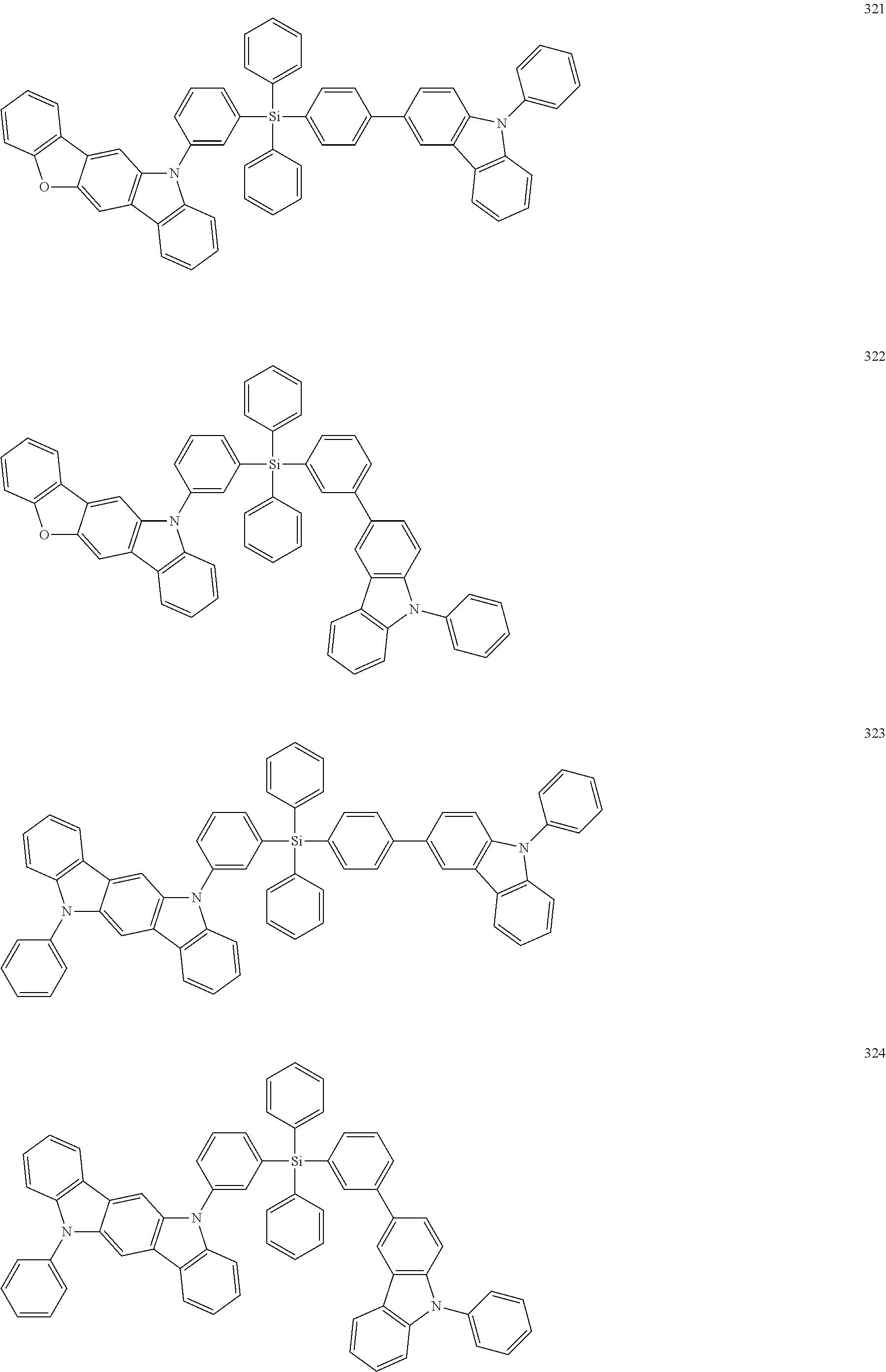

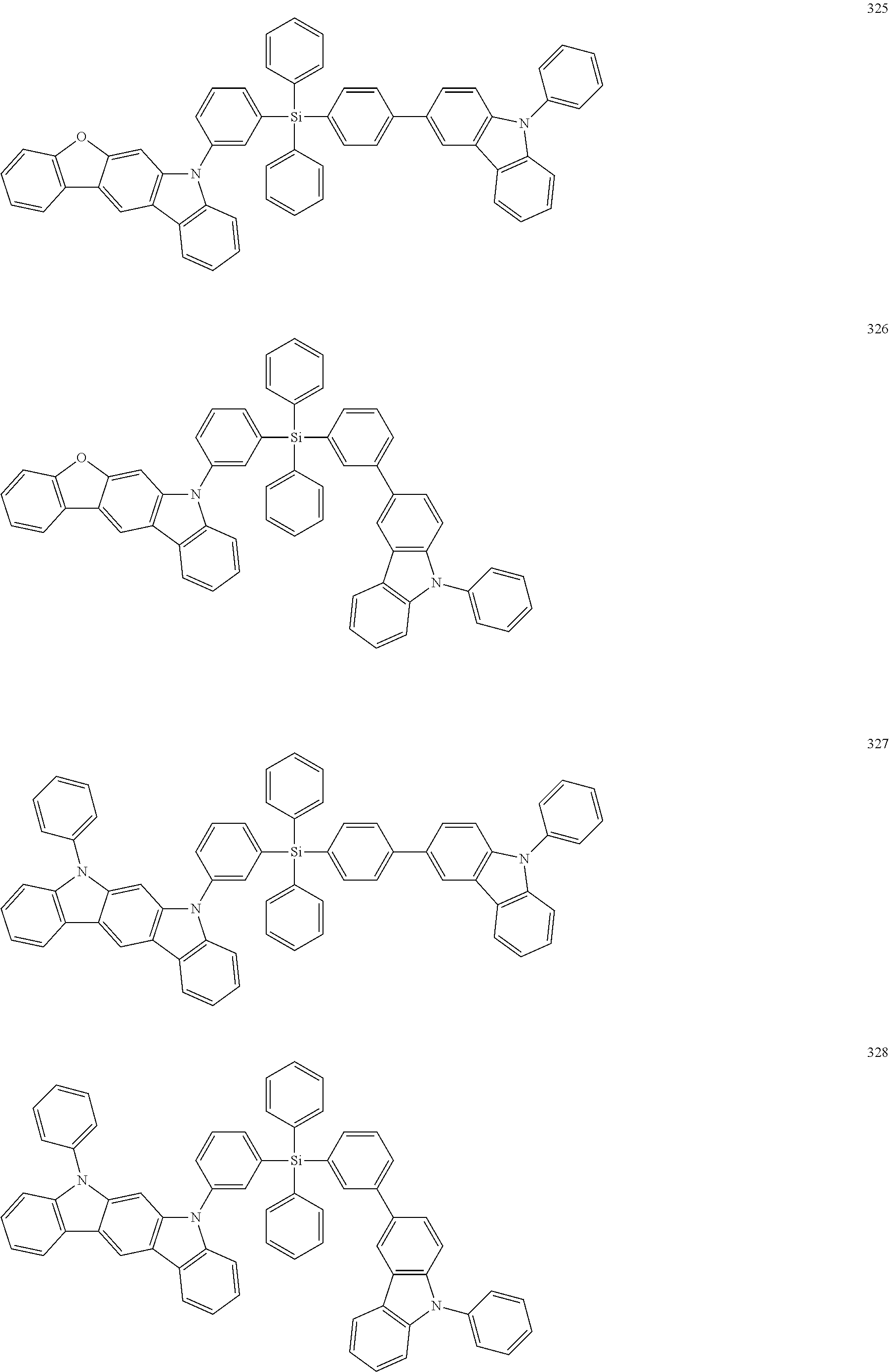

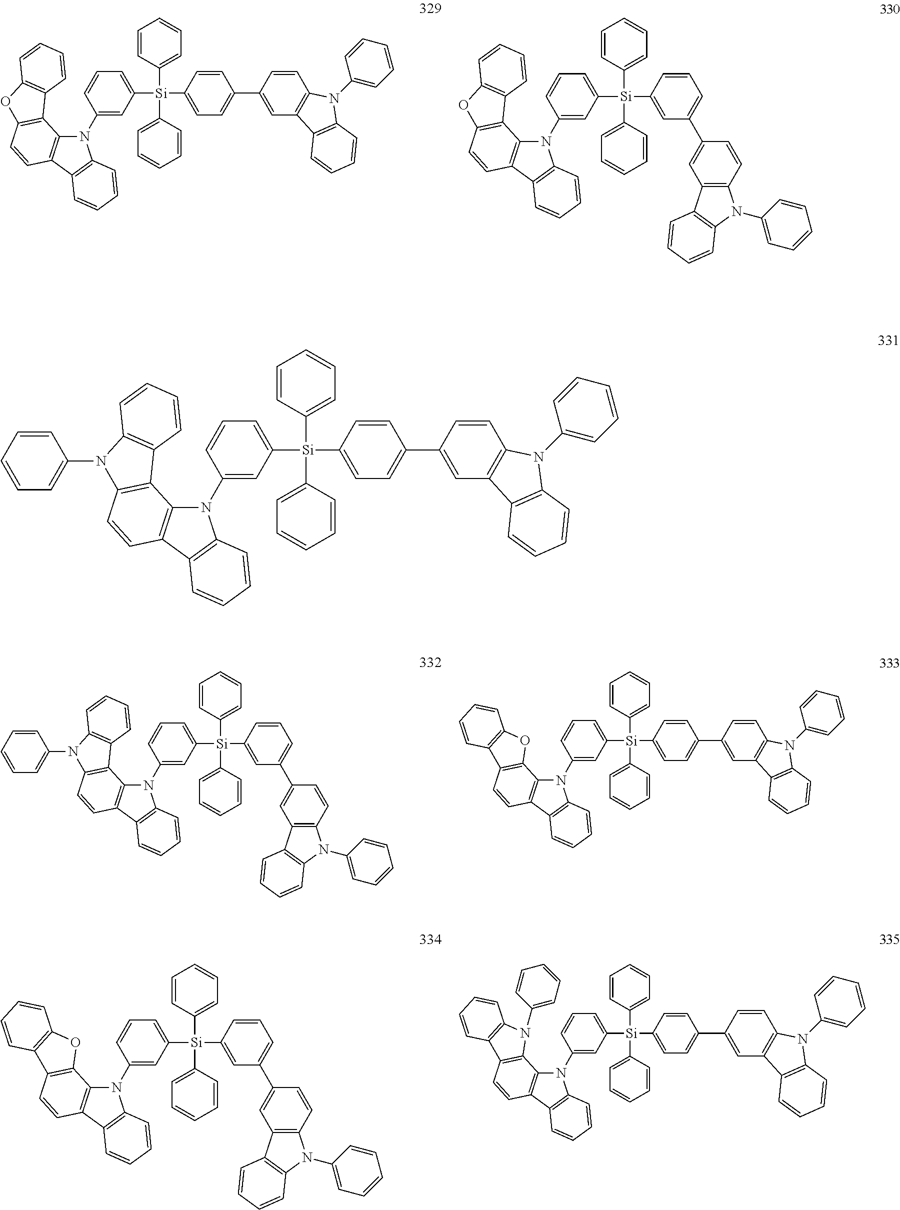

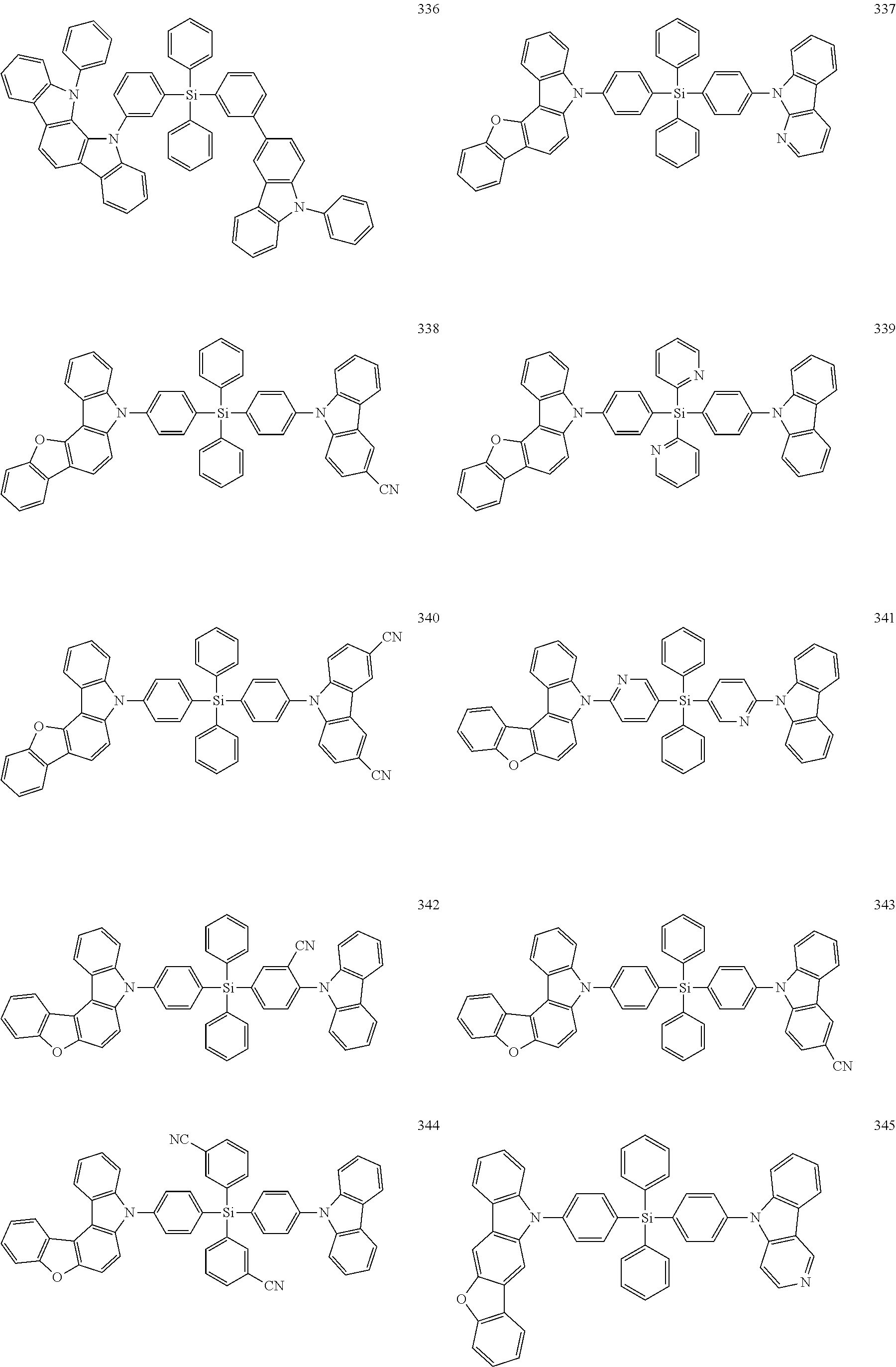

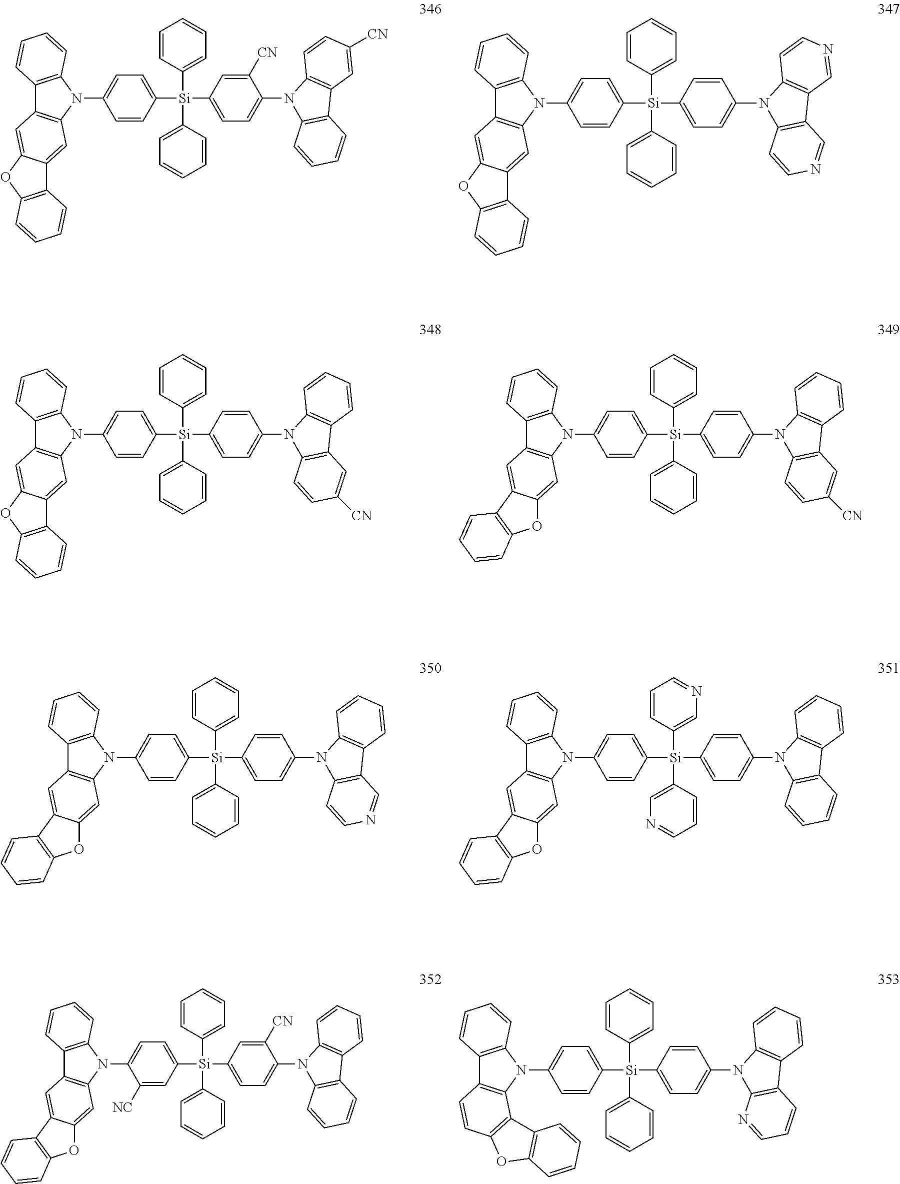

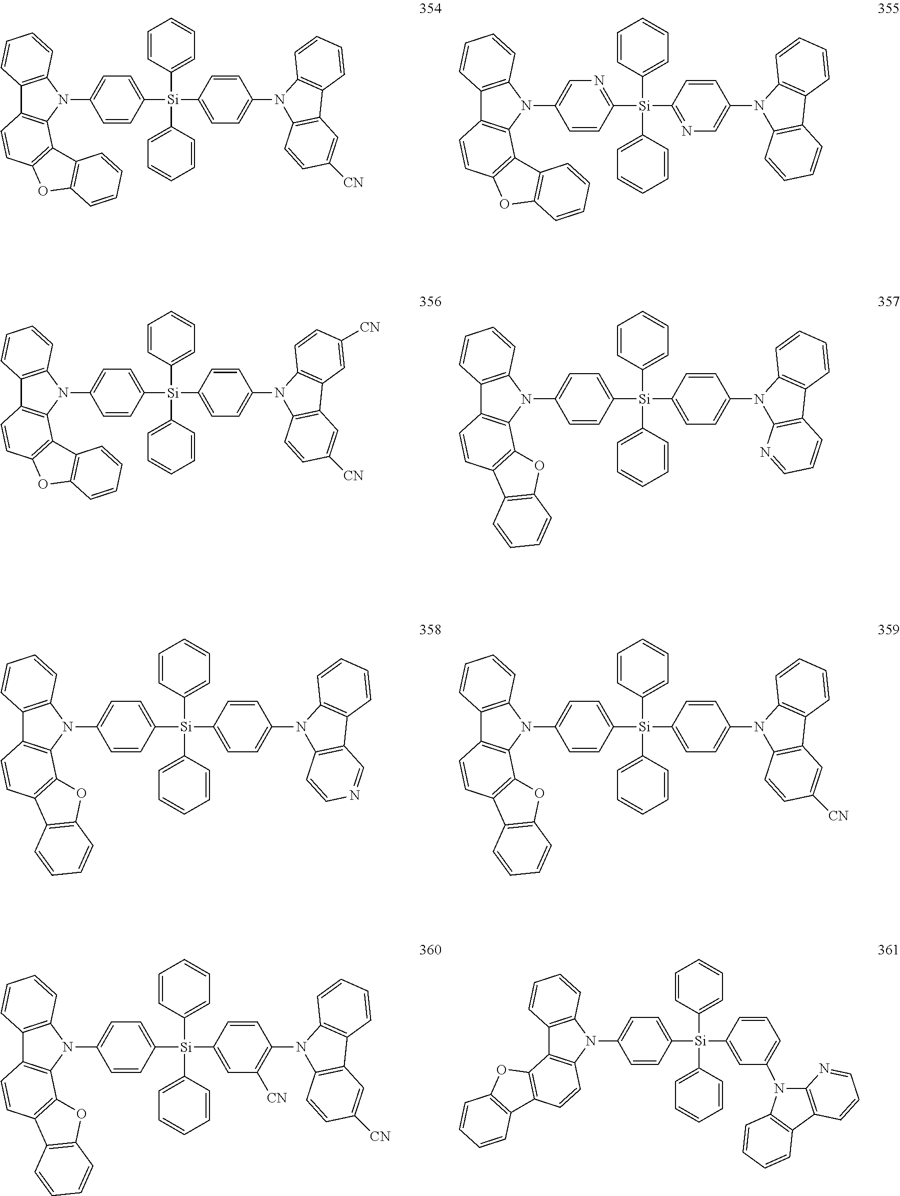

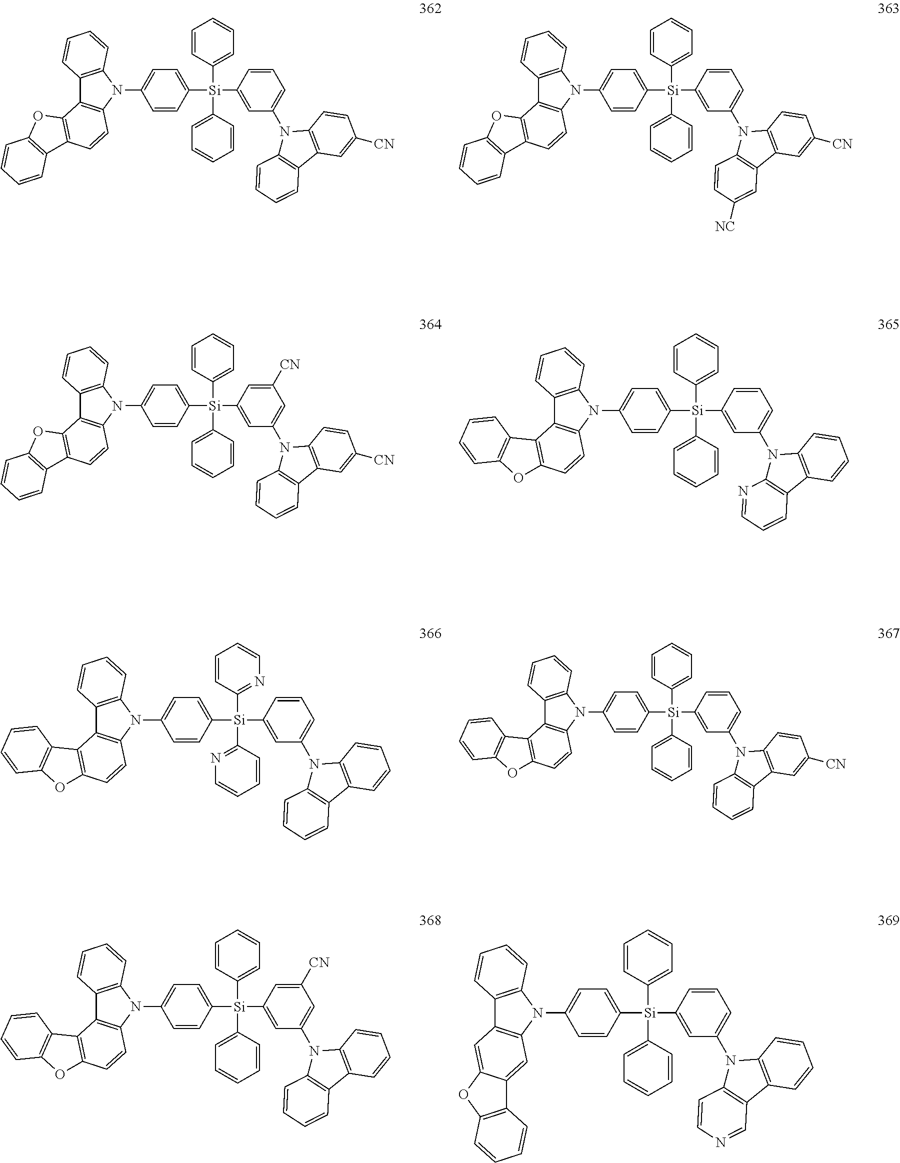

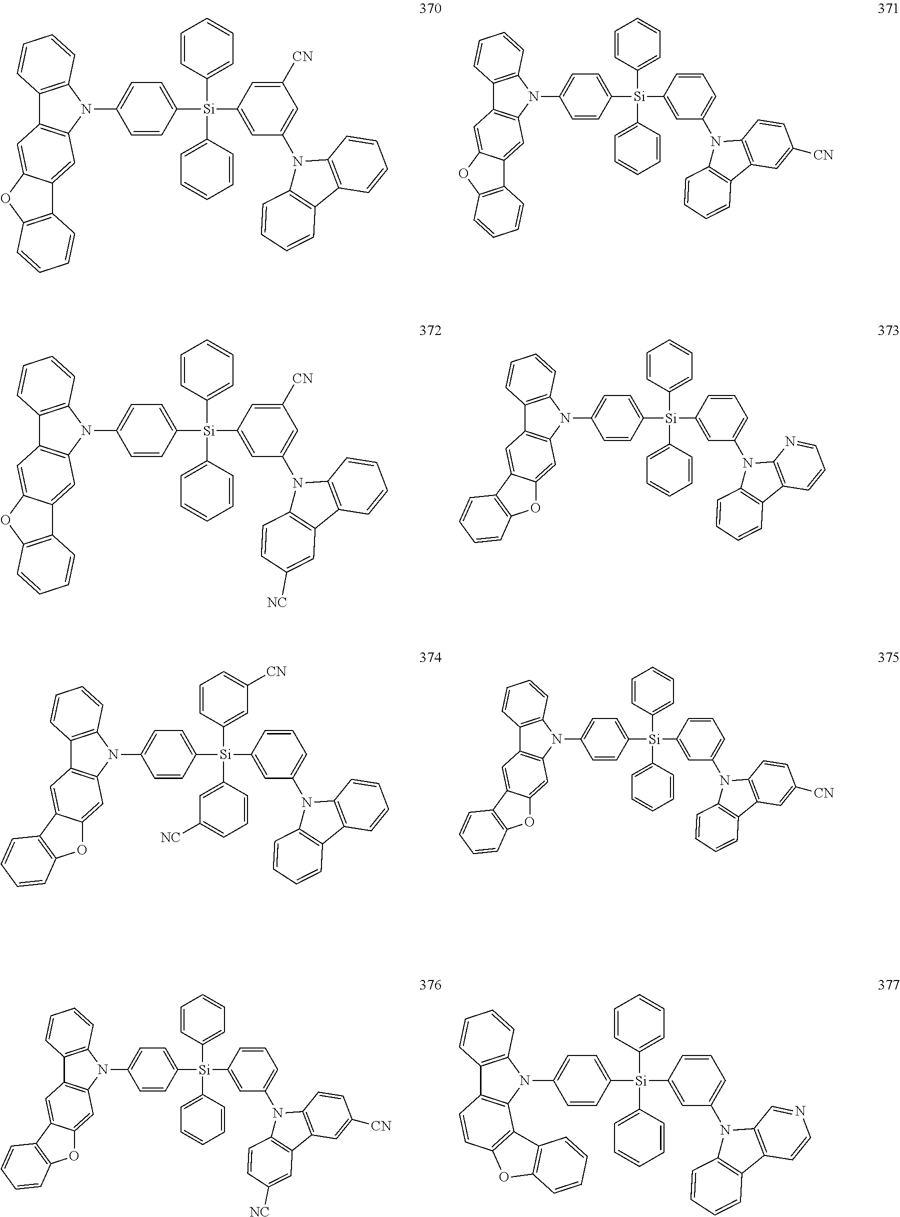

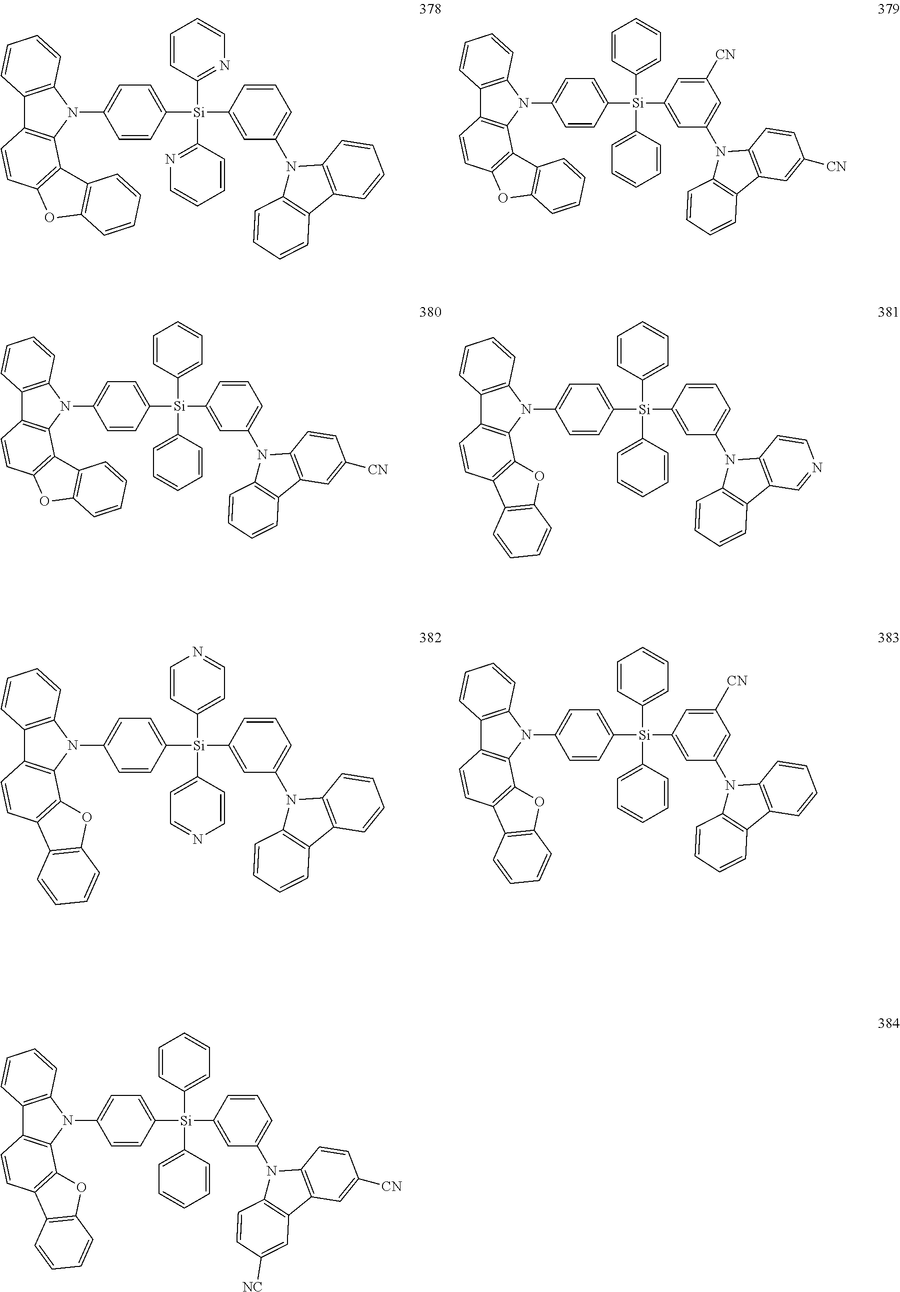

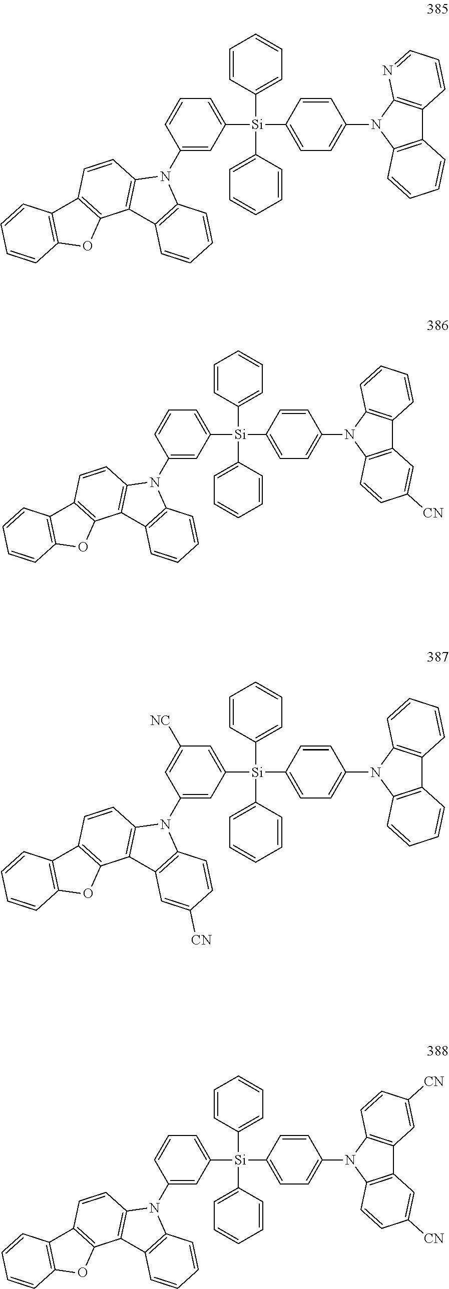

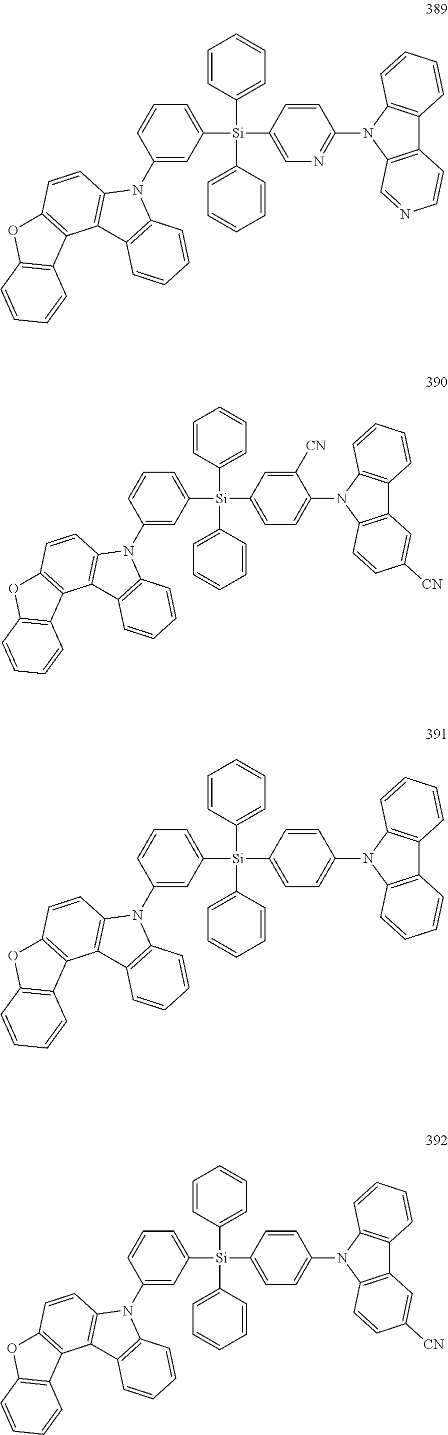

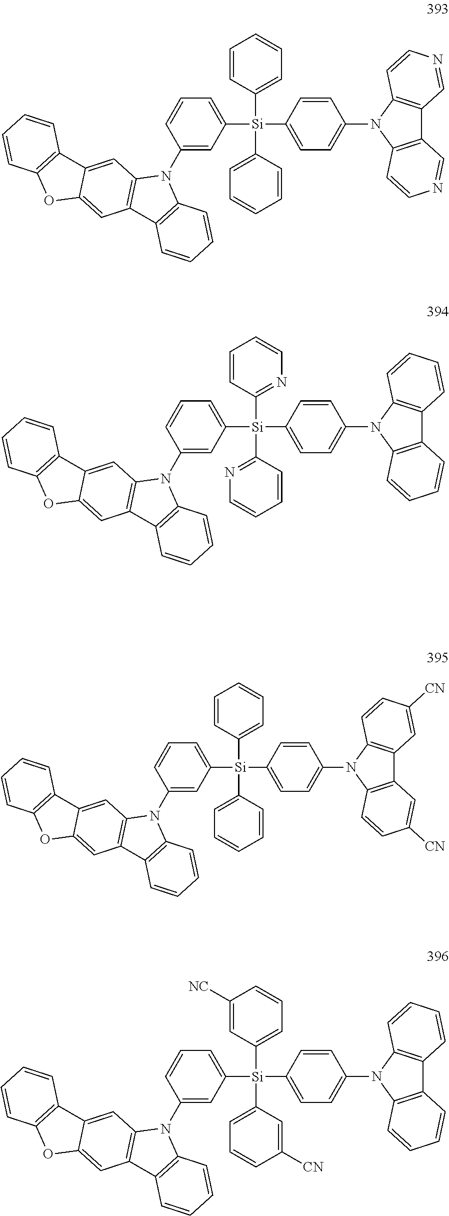

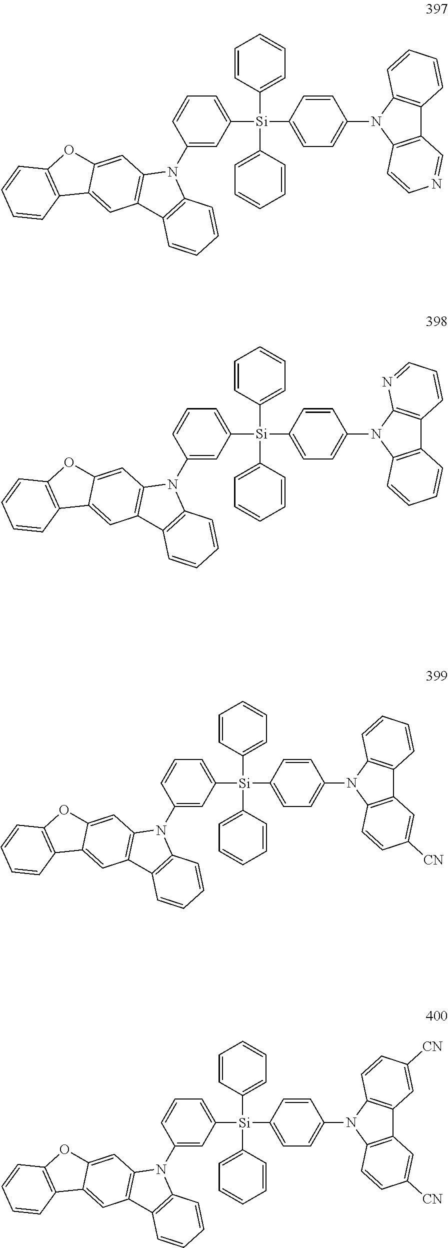

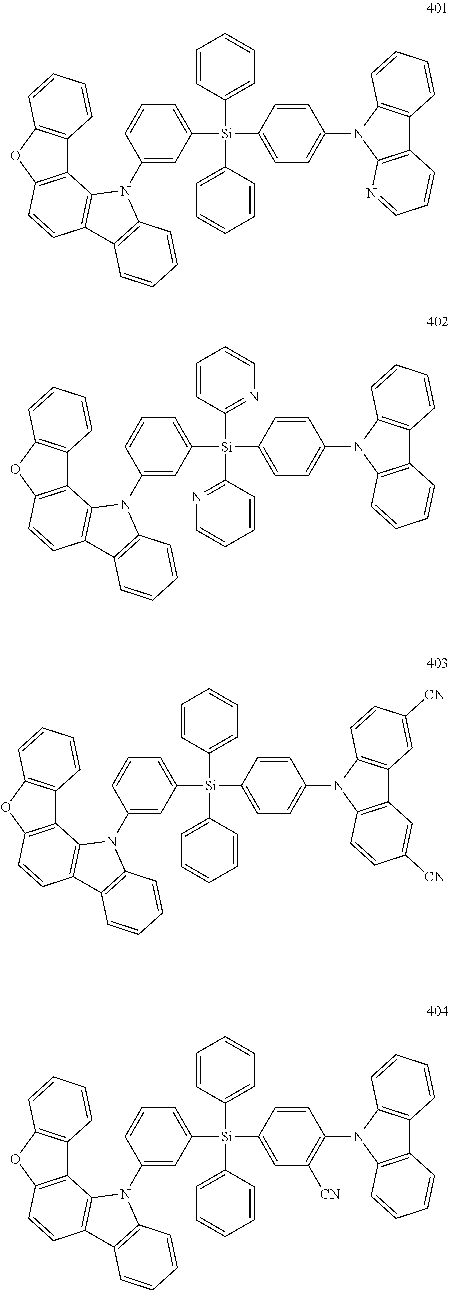

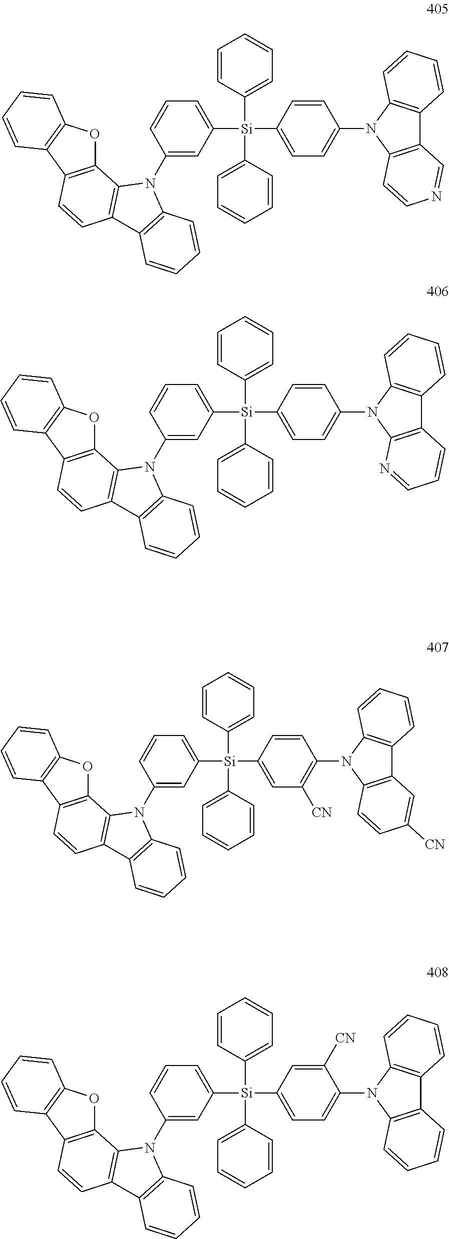

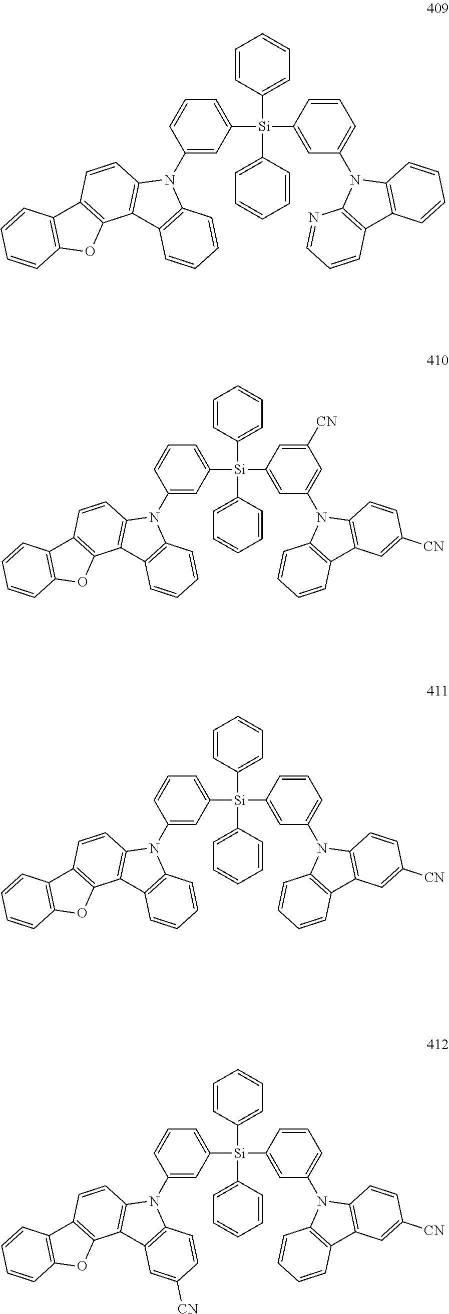

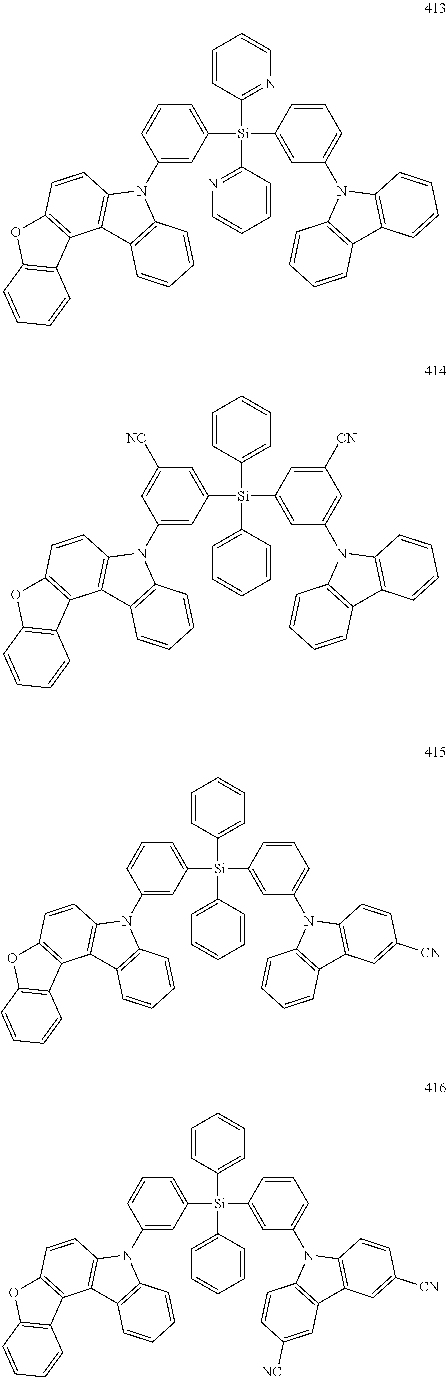

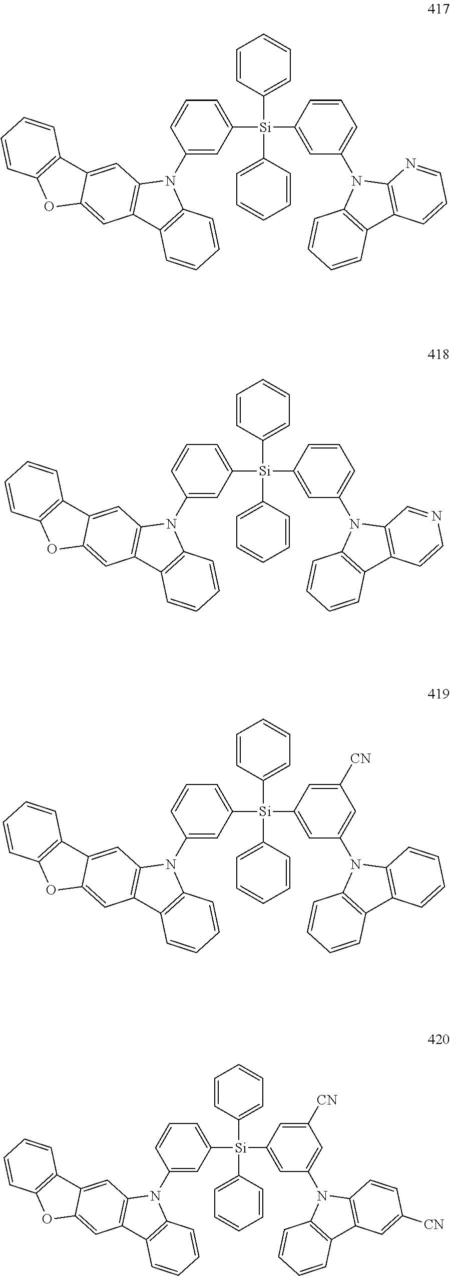

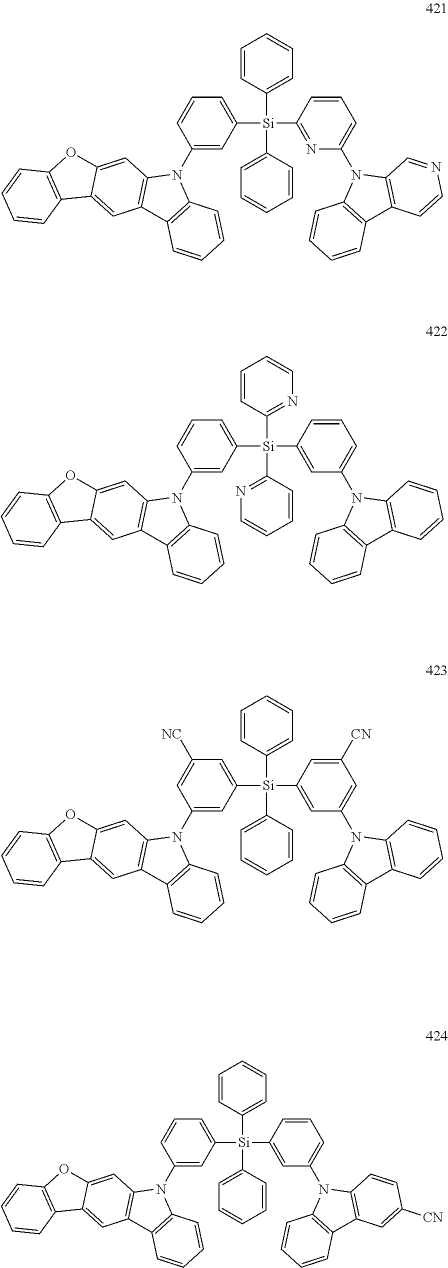

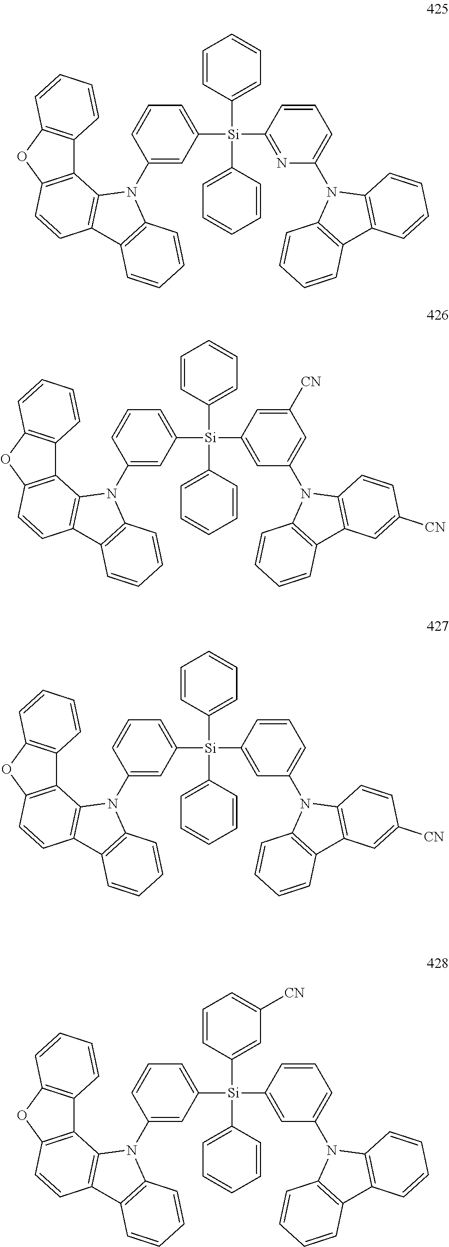

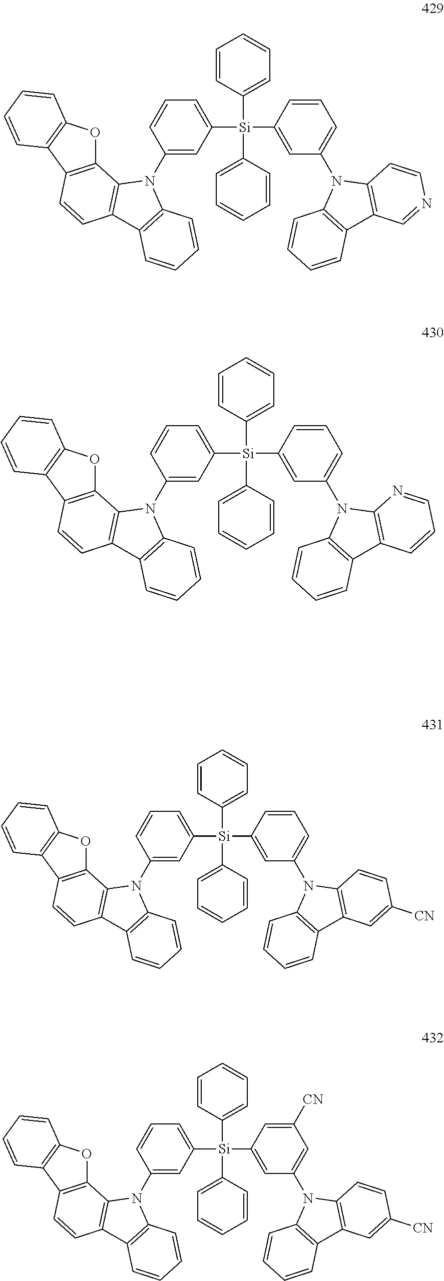

wherein, in Compounds 1 to 432,

Ph may be a phenyl group.

When the host is a mixture of an electron-transporting host and a hole-transporting host, the weight ratio of the electron-transporting host and the hole-transporting host may be about 1:9 to about 9:1, for example, about 2.8 to about 8:2, for example, about 4:6 to about 6:4, for example, about 5:5. When the weight ratio of the electron-transporting host and the hole-transporting host satisfies the above-described ranges, the hole-and-electron-transporting balance in the emission layer 15 may be made.

### Dopant in Emission Layer 15

The dopant may include the second compound.

Since the dopant emits fluorescent light, organic light-emitting devices according to one or more embodiments of the present disclosure are clearly distinguished from organic light-emitting devices containing compounds that emit phosphorescent light.

In one or more embodiments, the dopant may be free of metal atoms.

In one or more embodiments, the dopant may further include, in addition to the second compound, a compound that is a condensed polycyclic compound or a styryl compound.

For example, the dopant may include one of a naphthalene-containing core, a fluorene-containing core, a spiro-bifluorene-containing core, a benzofluorene-containing core, a dibenzofluorene-containing core, a phenanthrene-containing core, an anthracene-containing core, a fluoranthene-containing core, a triphenylene-containing core, a pyrene-containing core, a chrysene-containing core, a naphthacene-containing core, a picene-containing core, a perylene-containing core, a pentaphene-containing core, an indenoanthracene-containing core, a tetracene-containing core, a bianthracene-containing core, or core represented by one of Formulae 501-1 to 501-18, but embodiments of the present disclosure are not limited thereto:

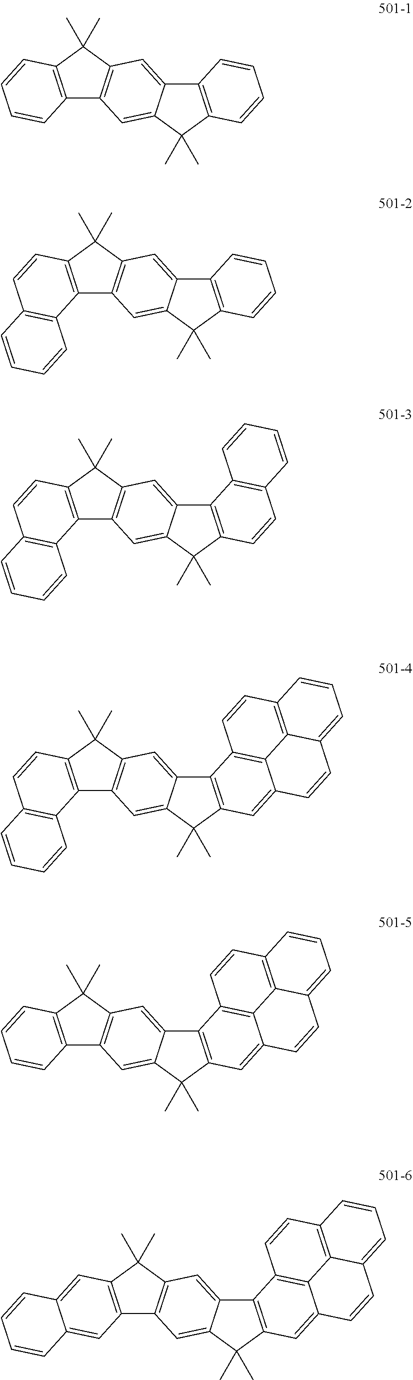

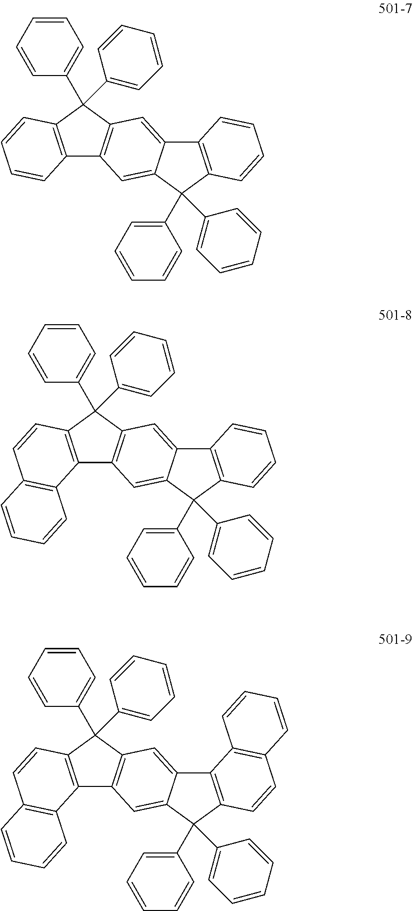

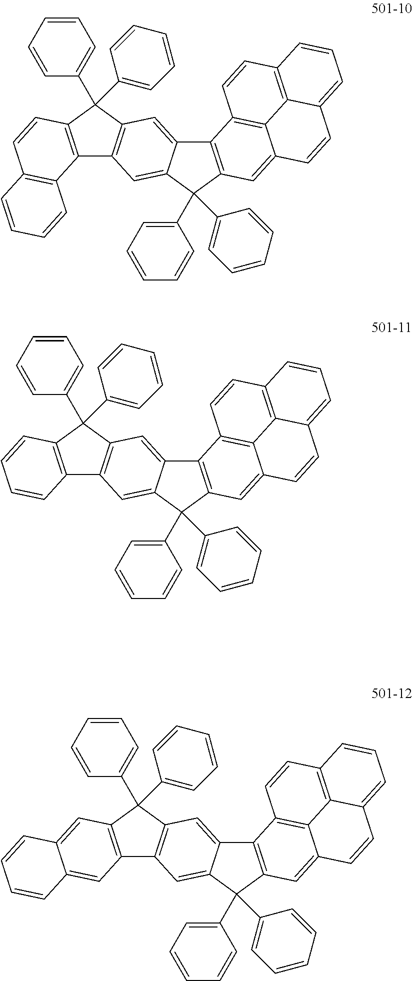

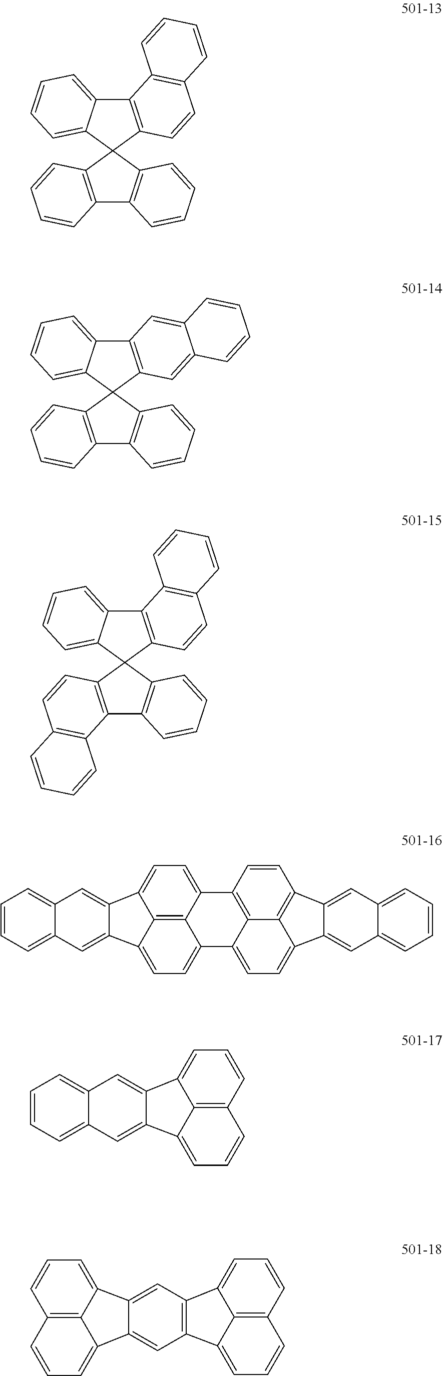

In one or more embodiments, the dopant may be a styryl-amine-containing compound or a styryl-carbazole-containing compound, but embodiments of the present disclosure are not limited thereto.

In one or more embodiments, the dopant may be a compound represented by Formula 501:

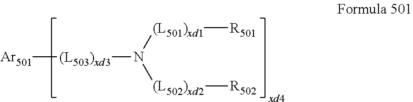

wherein, in Formula 501,

Ar₅₀₁ may be:

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a groups represented by one of Formulae 501-1 to 501-18: or

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene indenoanthracene group, a tetracene group, a bisanthracene group, or a group represented by one of Formulae 501-1 to 501-18, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, or —Si(Q₅₀₁)(Q₅₀₂)(Q₅₀₃) (wherein Q₅₀₁ to Q₅₀₃ may each independently be hydrogen, a C₁-C₆₀ alkyl group, a C₁-C₆₀ alkoxy group, a C₆-C₆₀ aryl group, a C₇-C₆₀ alky aryl group, a C₇-C₆₀ aryl alkyl group, a C₆-C₆₀ aryloxy group, a C₁-C₆₀ heteroaryl group, a C₂-C₆₀ alkyl heteroaryl group, a C₂-C₆₀ heteroaryl alkyl group, a C₁-C₆₀ heteroaryloxy group, a monovalent non-aromatic condensed polycyclic group, or a monovalent non-aromatic condensed heteropolycyclic group),

L₅₀₁ to L₅₀₃ may each independently be a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,

R₅₀₁ and R₅₀₂ may each independently be:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, or a dibenzothiophenyl group; or

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group,

xd1 to xd3 may each independently be 0, 1, 2, or 3, and

xd4 may be 0, 1, 2, 3, 4, 5, or 6.

For example, in Formula 501.

Ar₅₀₁ may be:

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by one of Formulae 501-1 to 501-18: or

a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by one of Formula 501-1 to 501-18, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a dibenzofuranyl group, a dibenzothiophenyl group, a carbazolyl group, a pyridinyl group, a pyrimidinyl group, a triazinyl group, a quinolinyl group, an isoquinolinyl group, or —Si(Q₅₀₁)(Q₅₀₂)(Q₅₀₃) (wherein Q₅₀₁ to Q₅₀₃ may each independently be hydrogen, a C₁-C₂₀ alkyl group, a C₁-C₂₀ alkoxy group, a phenyl group, a biphenyl group, a terphenyl group, or a naphthyl group),

L₅₀₁ to L₅₀₃ are the same as described in connection with L₂₀₁ to L₂₀₉,

xd1 to xd3 may each independently be 0, 1, or 2, and

xd4 may be 0, 1, 2, or 3, but embodiments of the present disclosure are not limited thereto.

In one or more embodiments, the dopant may include a compound represented by one of Formulae 502-1 to 502-5:

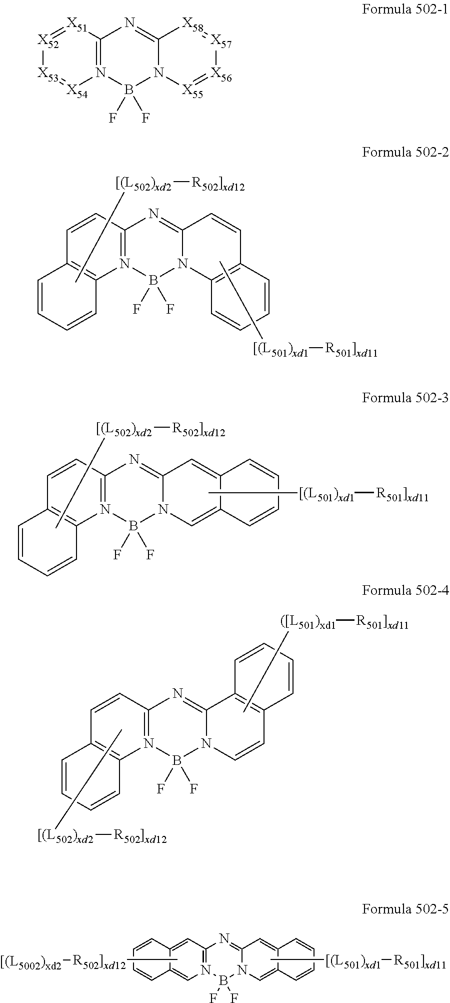

wherein, in Formulae 502-1 to 502-5,

X₅₁ may be N or C-[(L₅₀₁)_{xd1}-R₅₀₁], X₅₂ may be N or C-[(L₅₀₂)_{xd2}-R₅₀₂], X₅₃ may be N or C-[(L₅₀₃)_{xd3}-R₅₀₃], X₅₄ may be N or C-[(L₅₀₄)_{xd4}-R₅₀₄], X₅₅ may be N or C-[(L₅₀₅)_{xd5}-R₅₀₅], X₅₆ may be N or C-[(L₅₀₆)_{xd6}-R₅₀₆], X₅₇ may be N or C-[(L₅₀₇)_{xd7}-R₅₀₇], and X₅₈ may be N or C-[(L₅₀₈)_{xd8}-R₅₀₈],

L₅₀₁ to L₅₀₈ are each the same as described in connection with L₅₀₁ in Formula 501,

xd1 to xd8 are each the same as described in connection with xd1 in Formula 501.

R₅₀₁ to R₅₀₈ may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group,

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, or a dibenzothiophenyl group; or

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group,

xd11 and xd12 may each independently be an integer from 0 to 5,

two of R₅₀₁ to R₅₀₄ may optionally be linked together to form a saturated or unsaturated ring, and

two of R₅₀₅ to R₅₀₈ may optionally be linked together to form a saturated or unsaturated ring.

The dopant may include, for example, at least one of Compounds FD(1) to FD(16) or FD1 to FD18:

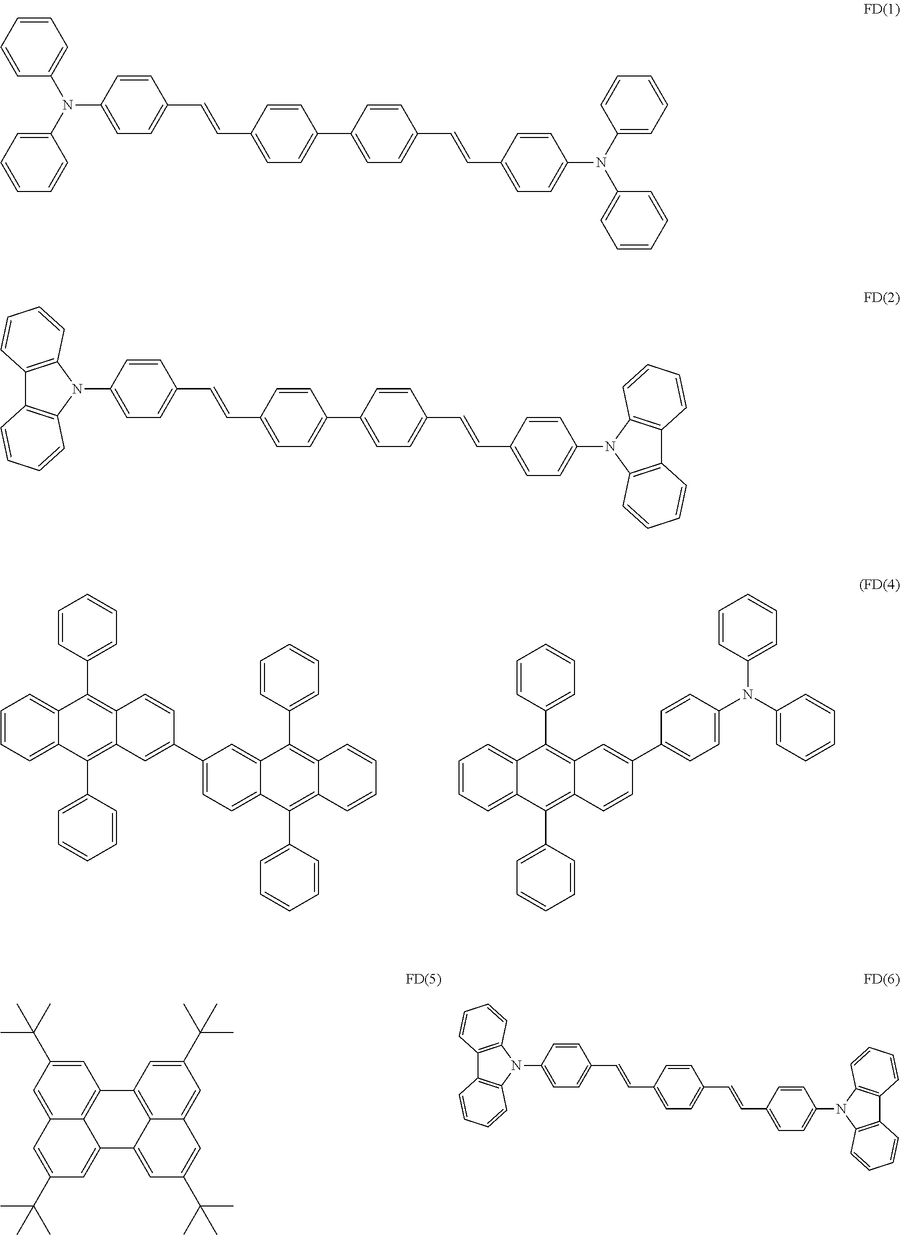

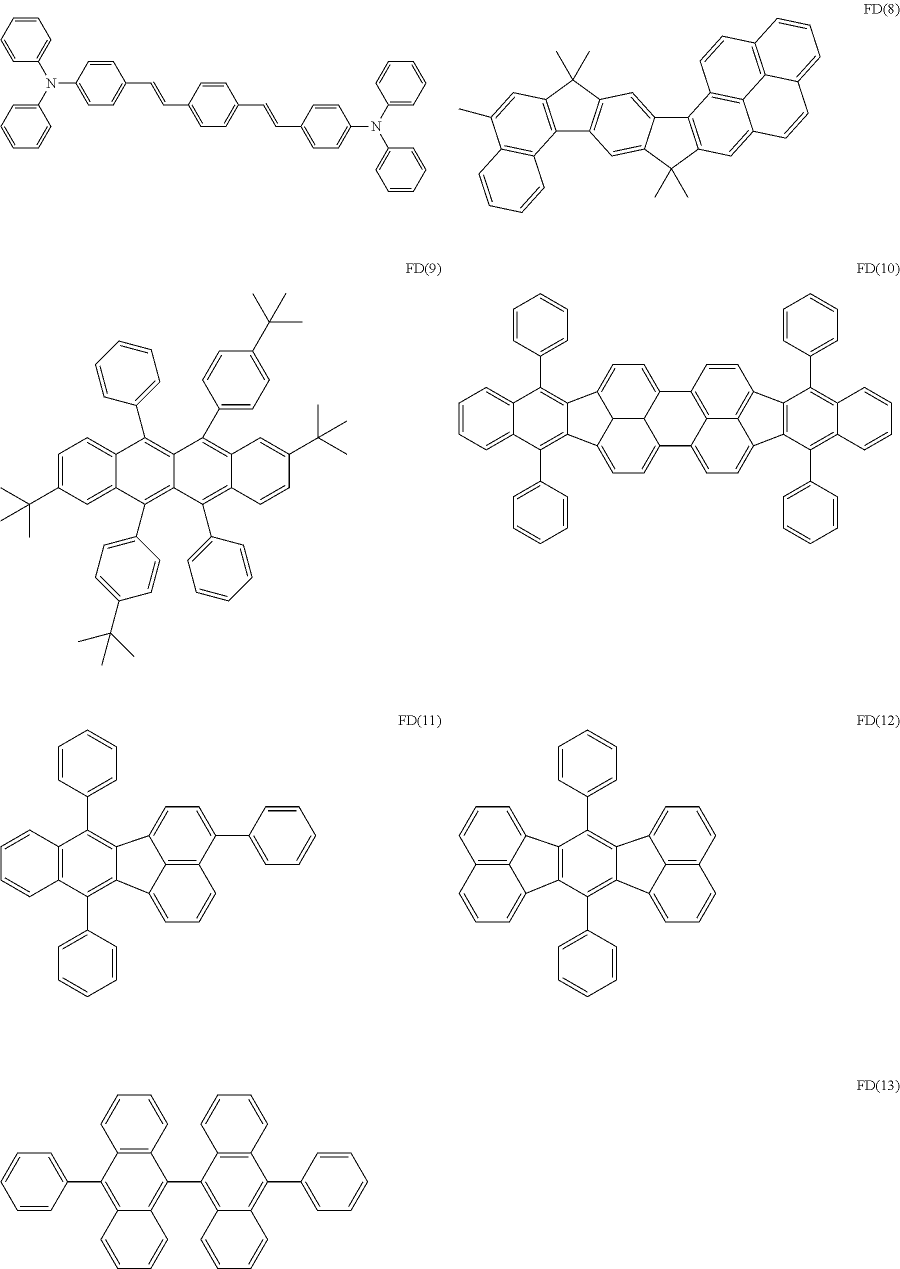

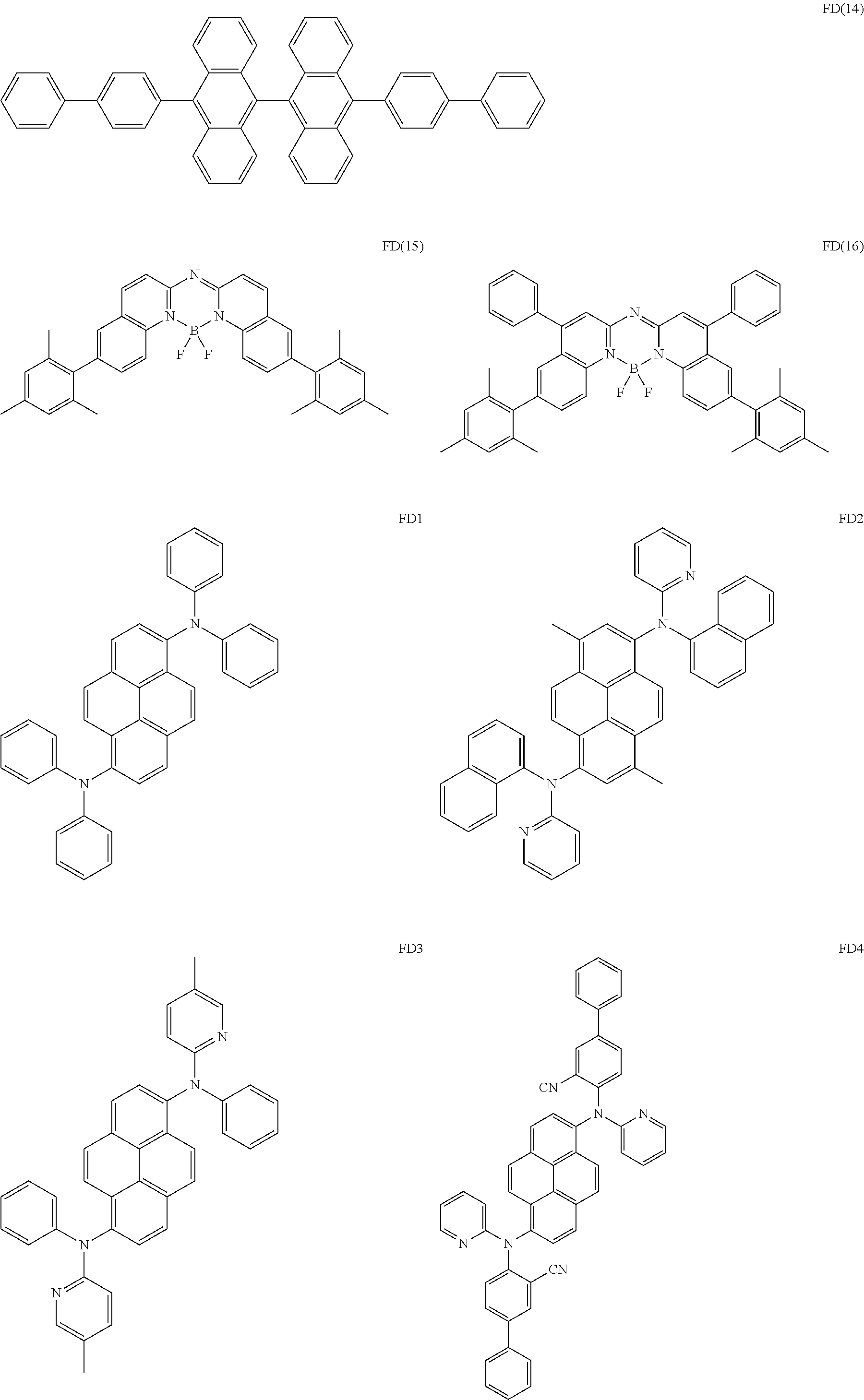

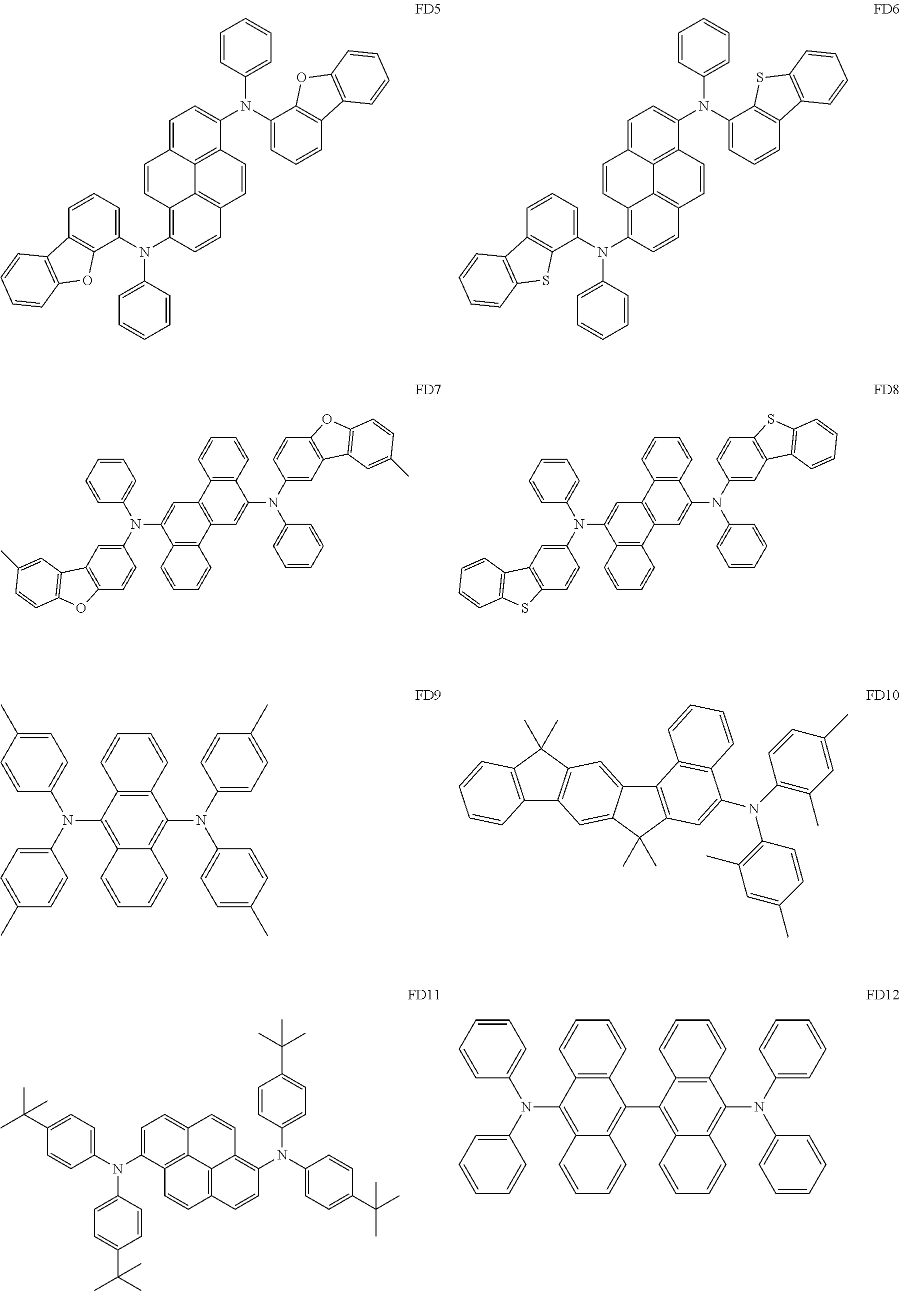

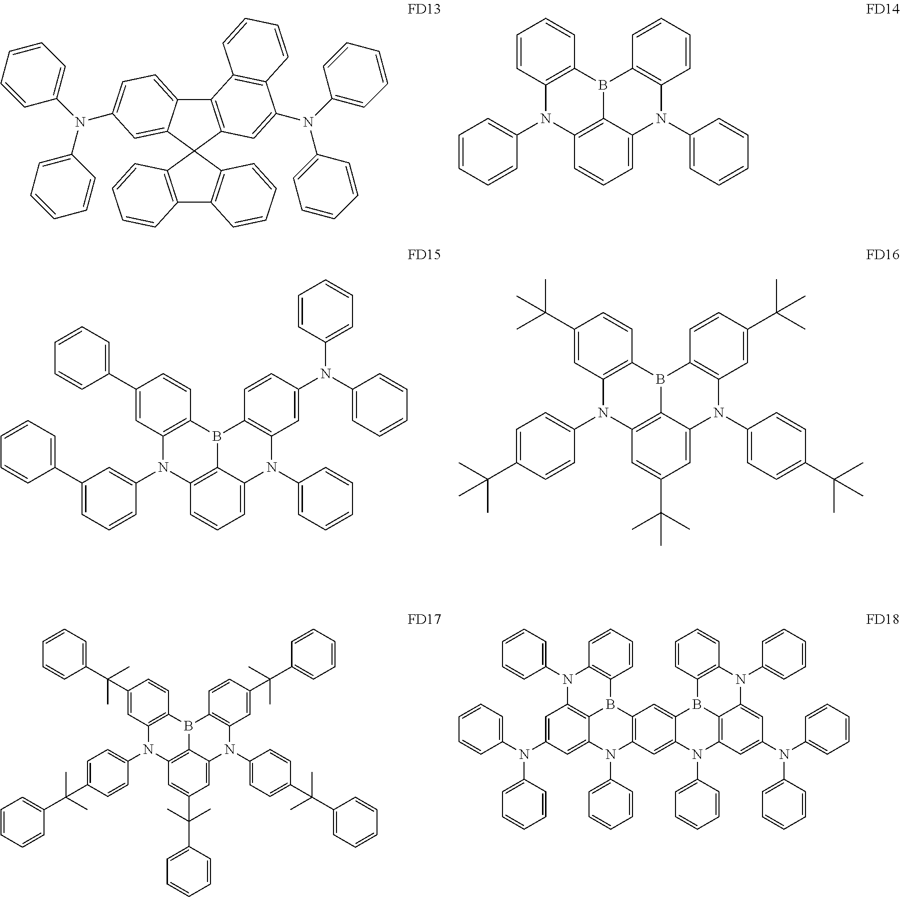

The amount of the dopant in the emission layer may be about 0.01 wt % to about 15 wt %, but embodiments of the present disclosure are not limited thereto.

### Sensitizer in Emission Layer 15

The sensitizer may include the first compound.

In one or more embodiments, the sensitizer may be a compound from Groups I to VI below, but embodiments of the present disclosure are not limited thereto:

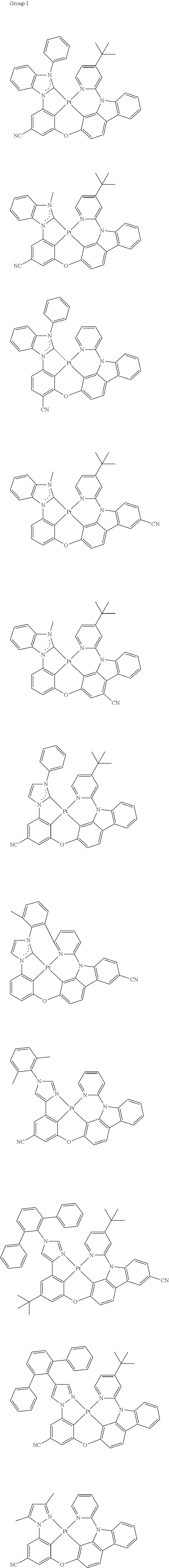

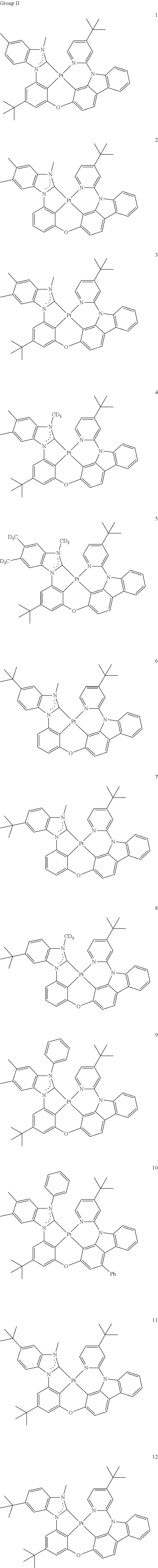

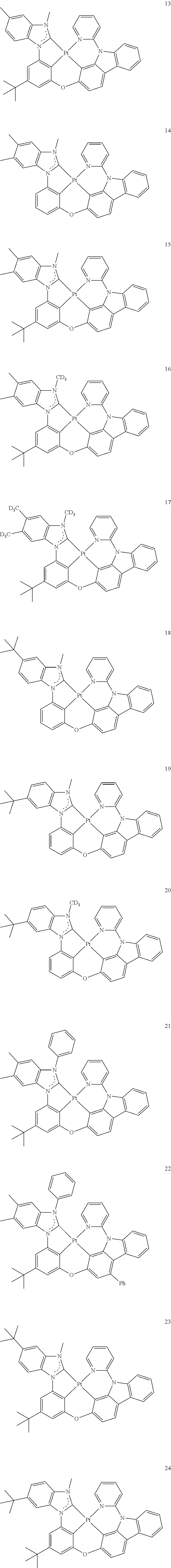

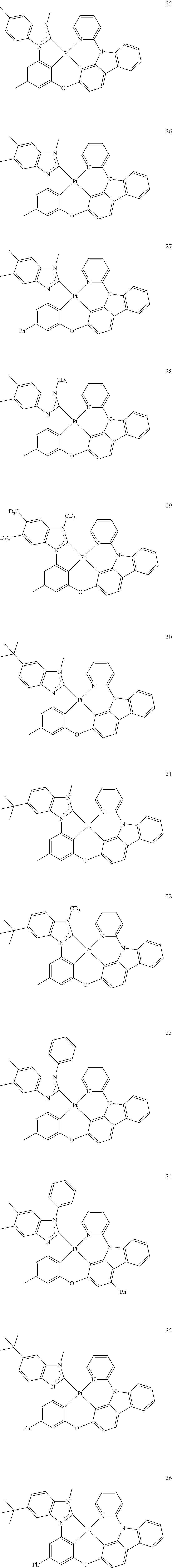

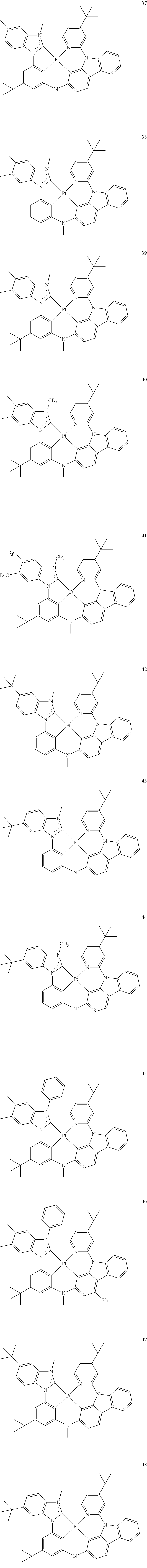

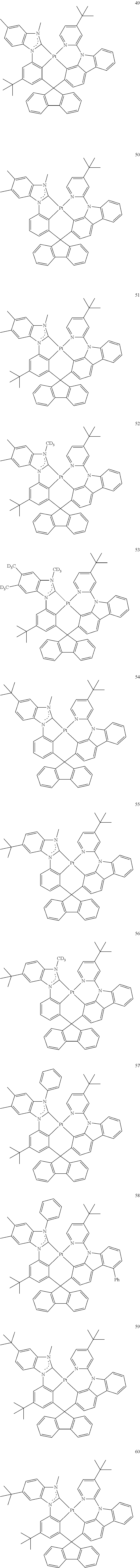

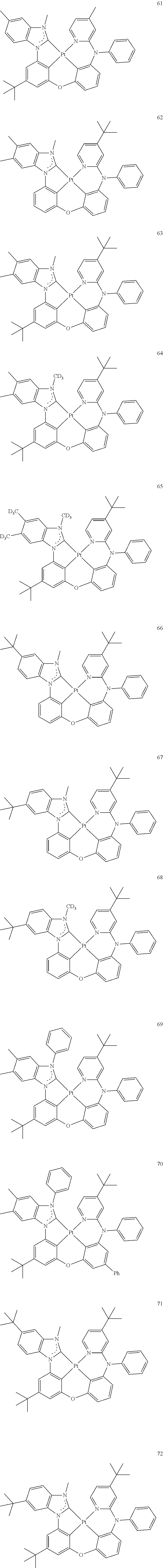

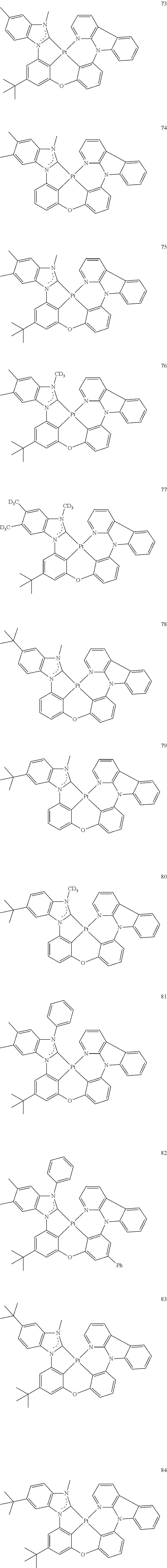

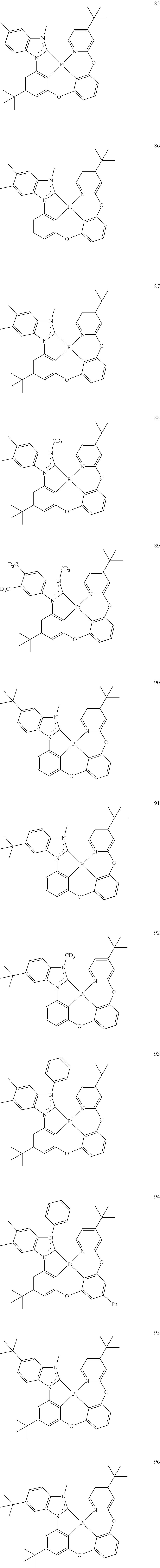

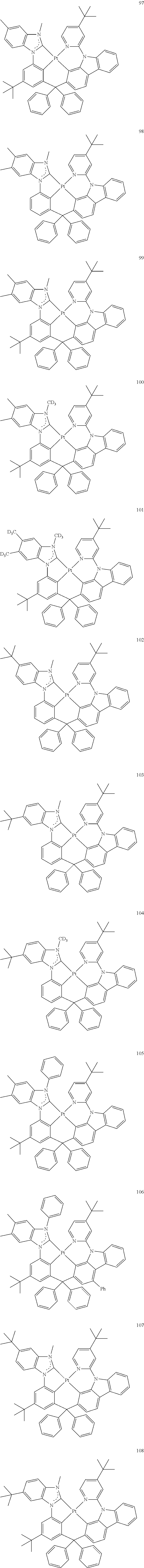

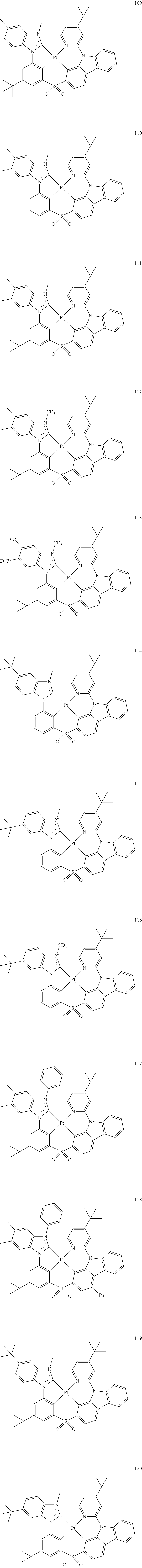

[chemical structure]

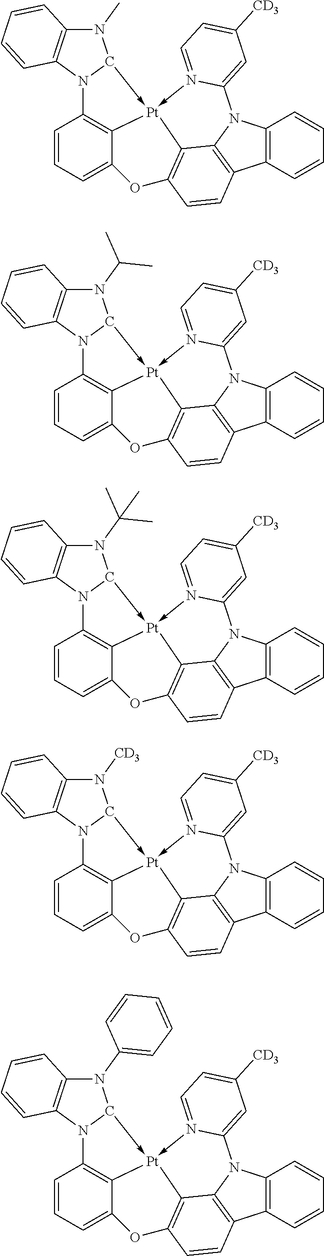

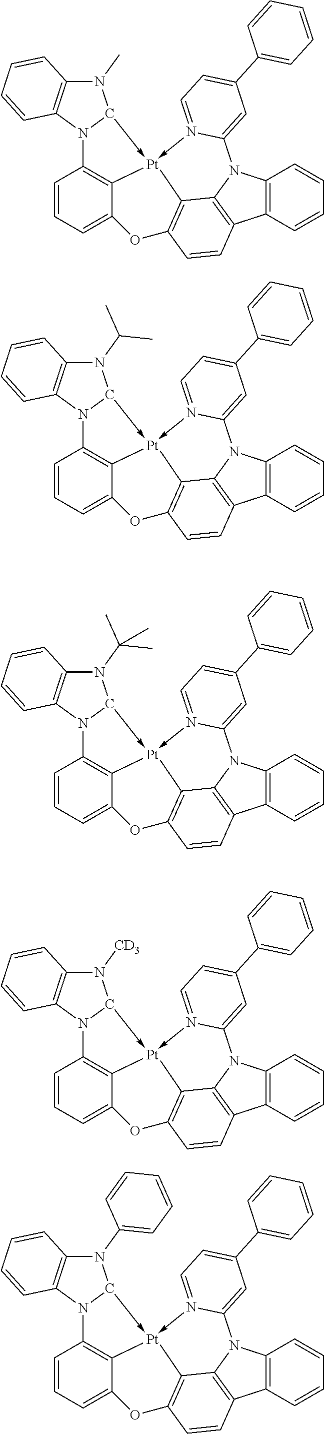

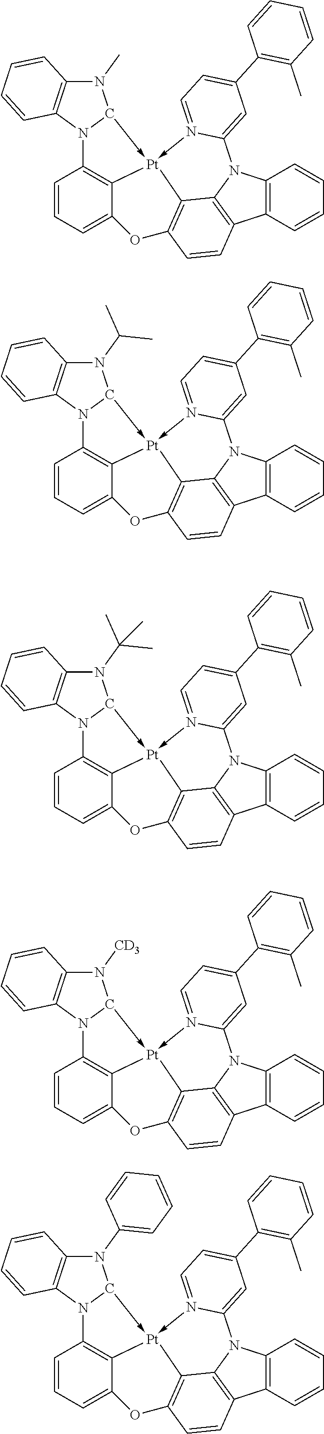

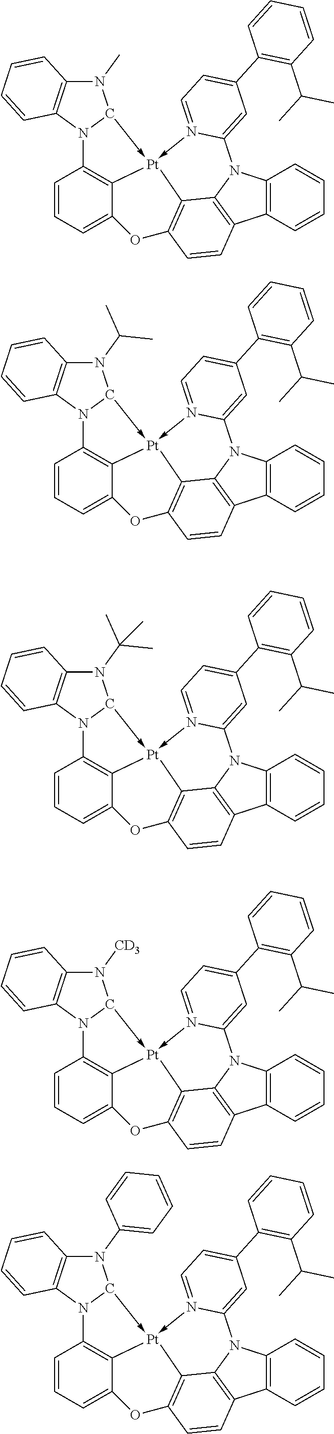

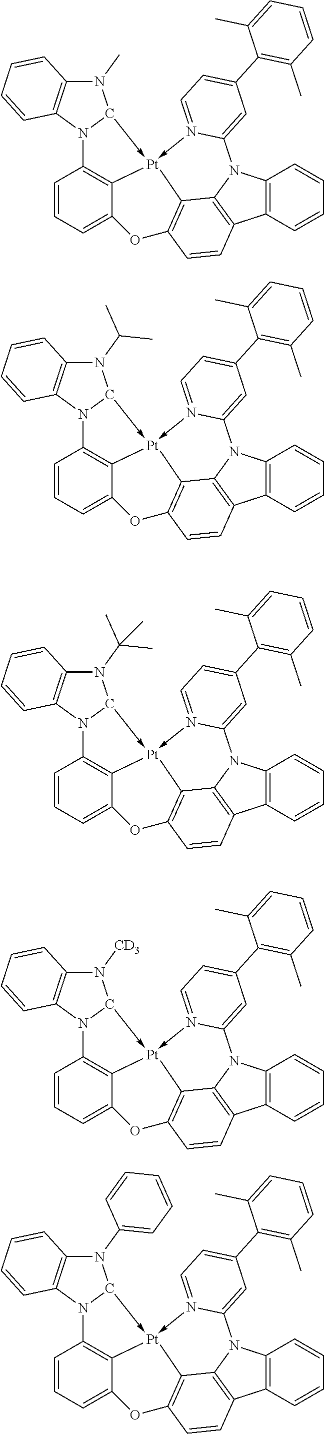

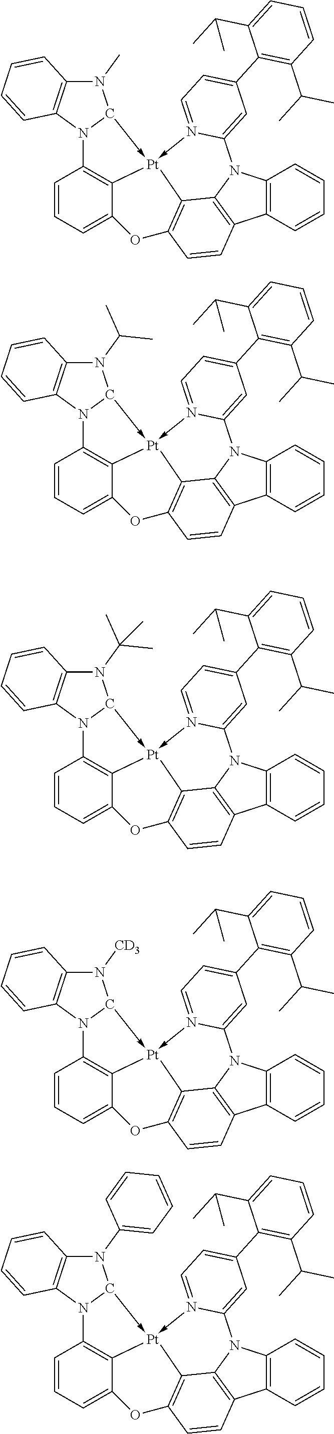

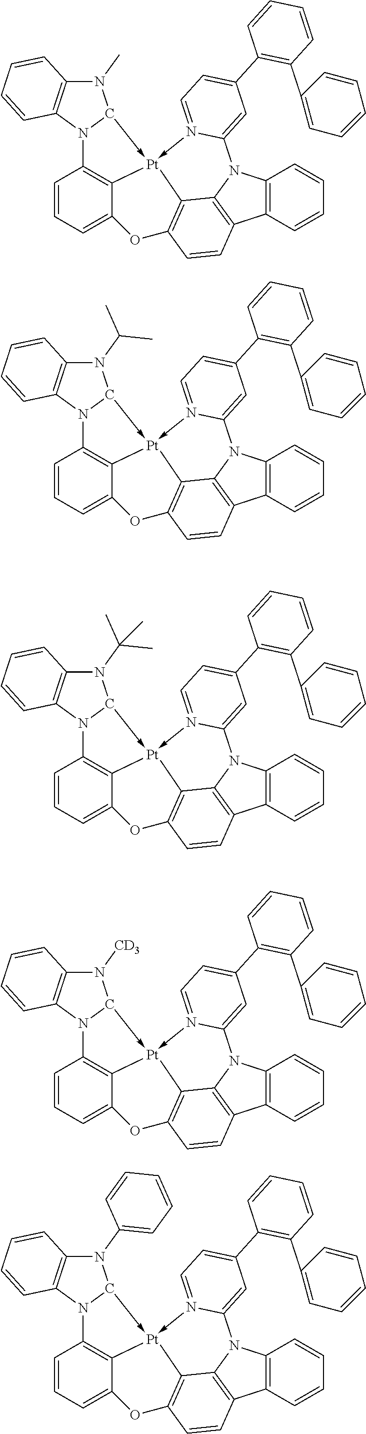

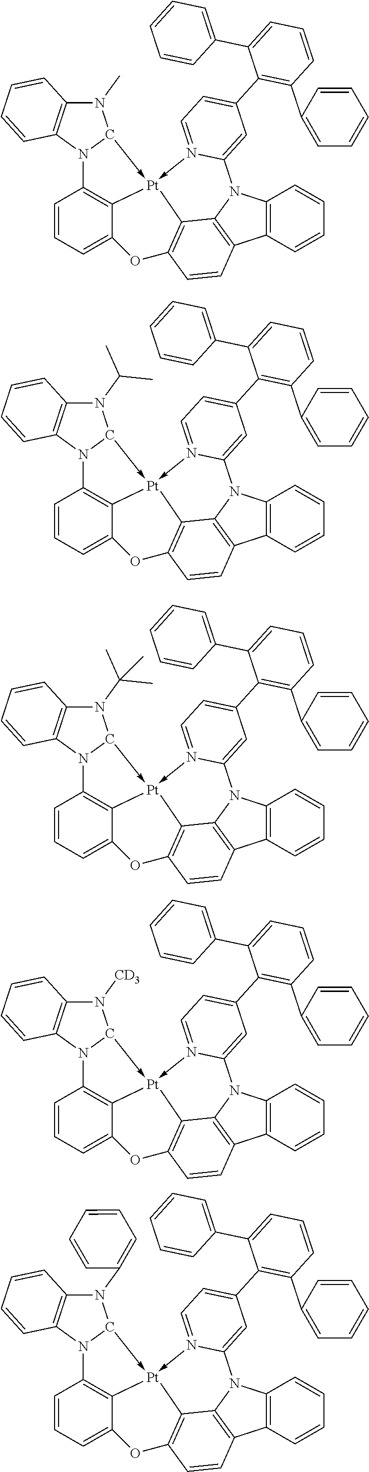

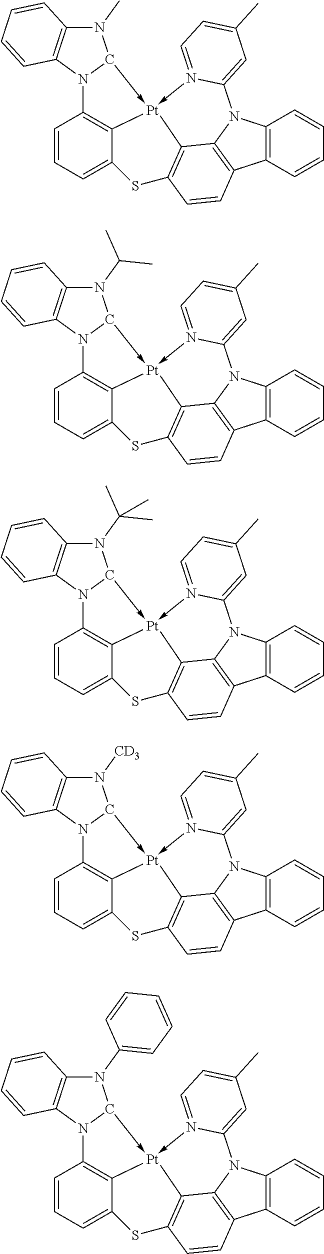

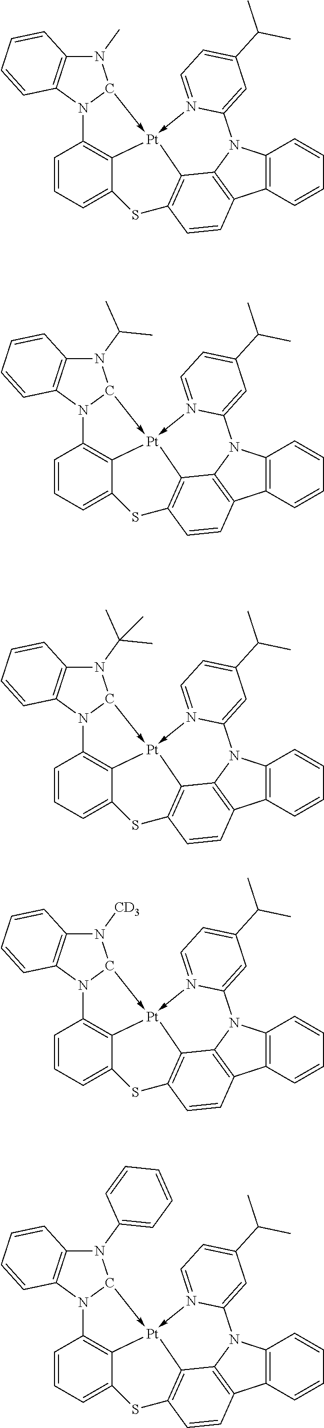

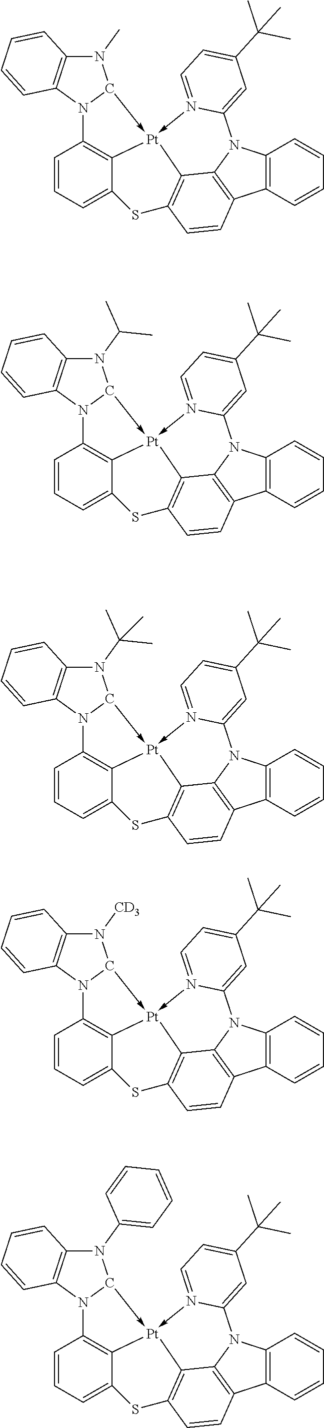

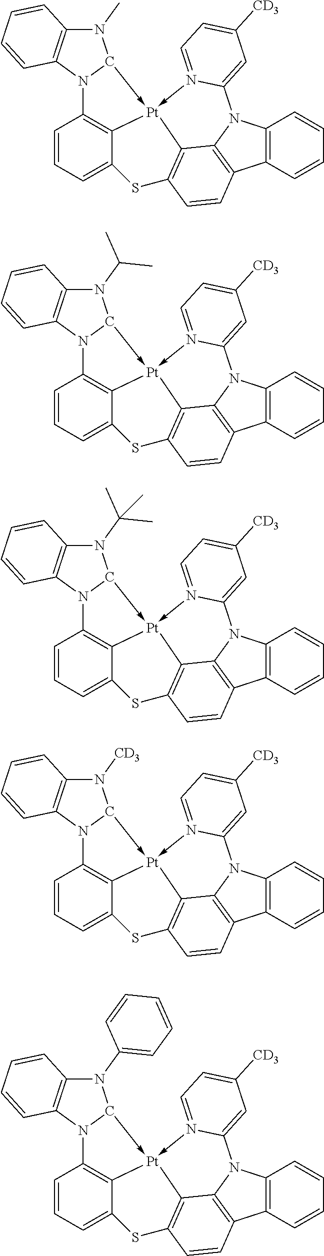

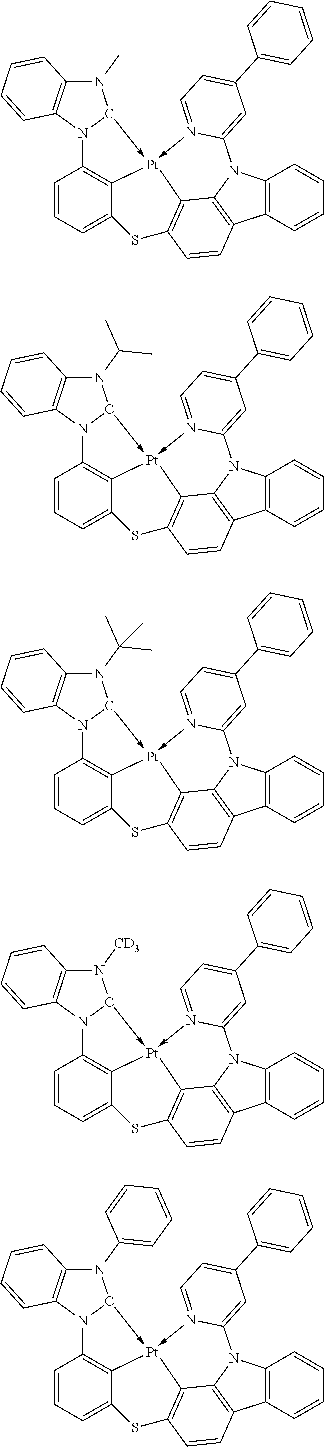

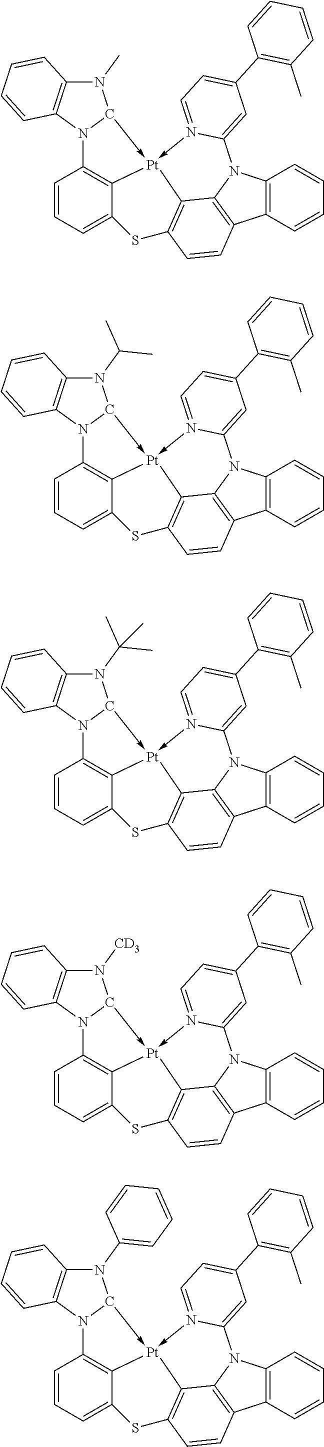

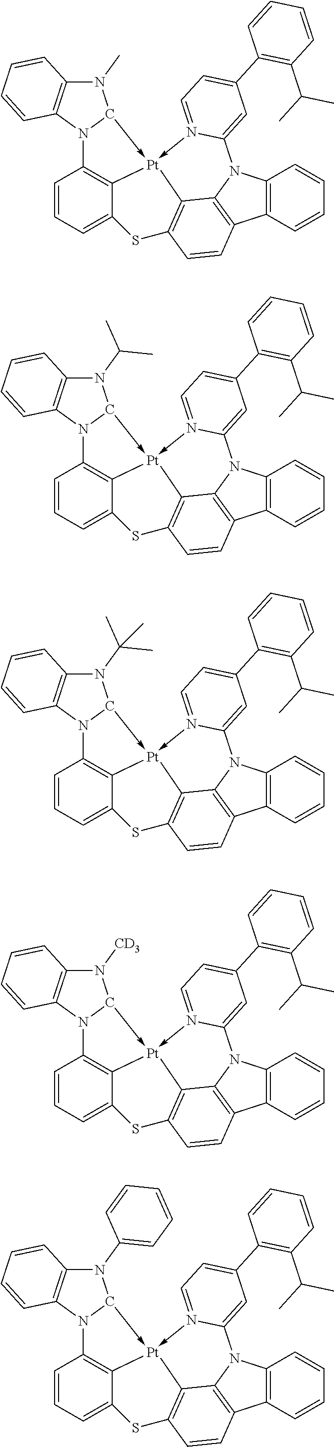

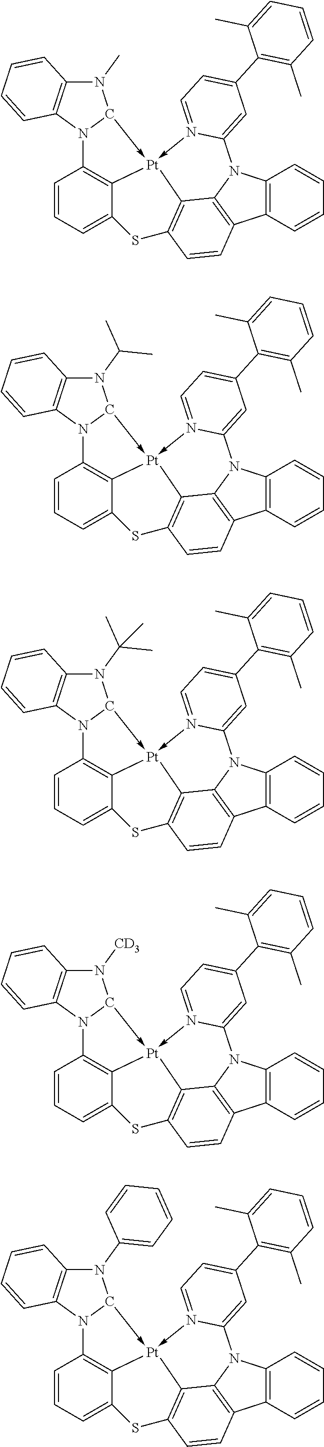

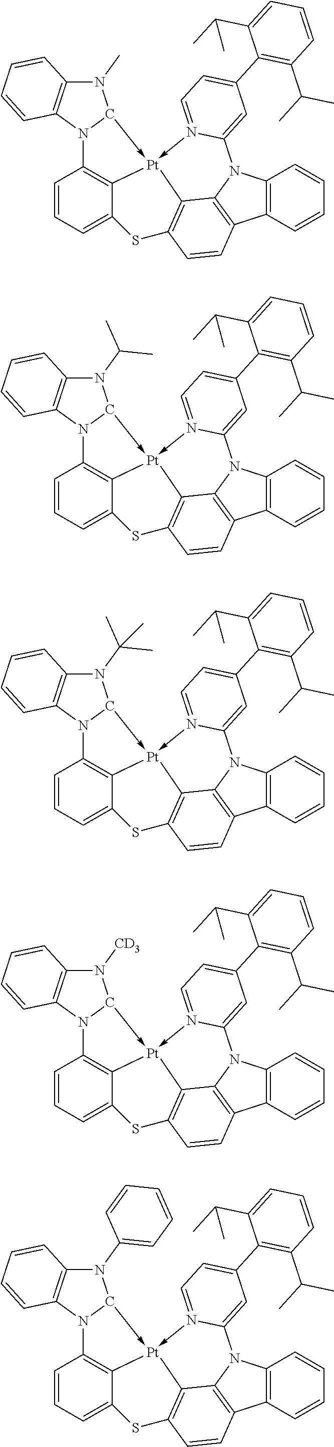

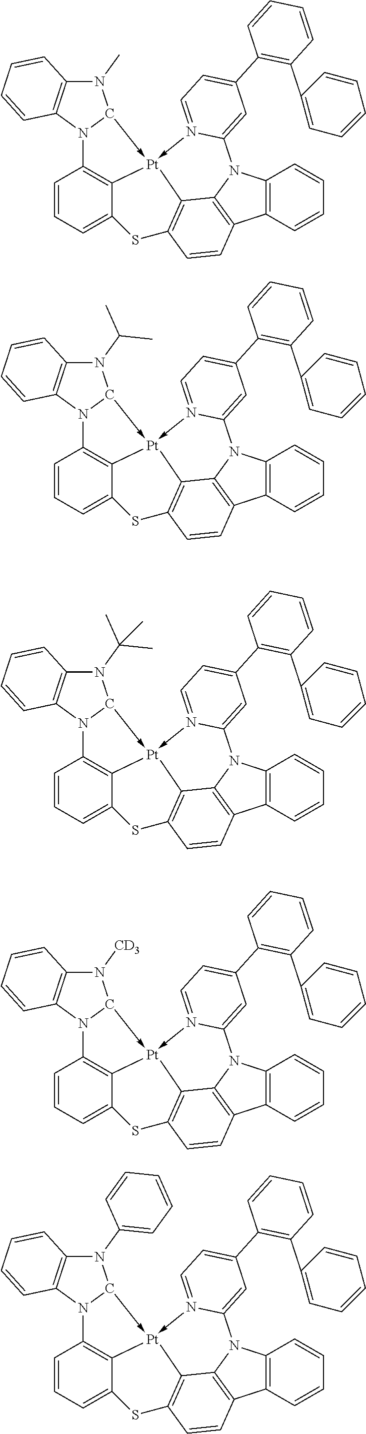

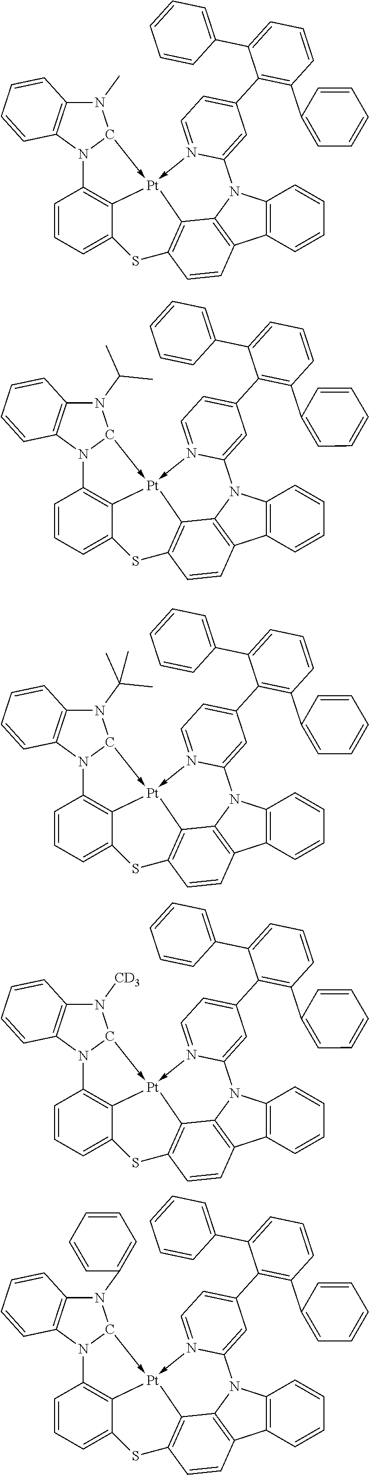

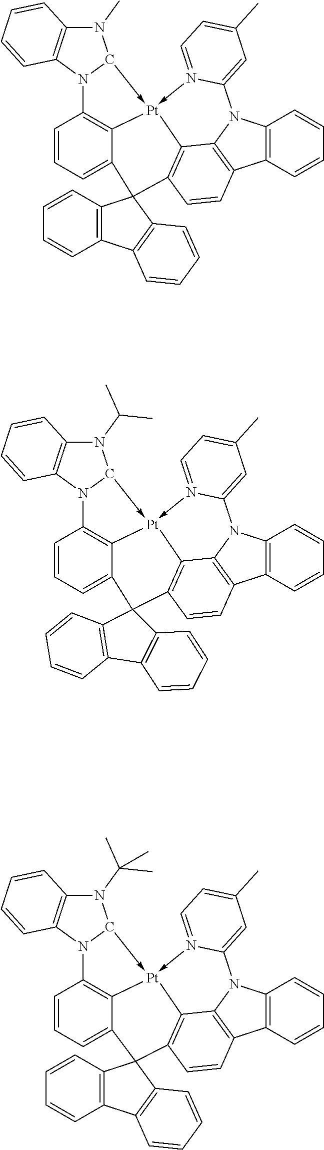

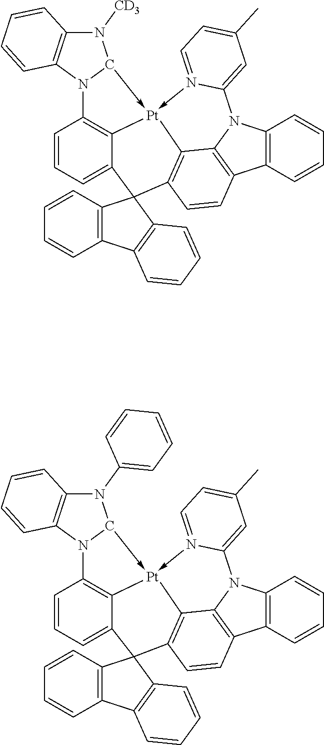

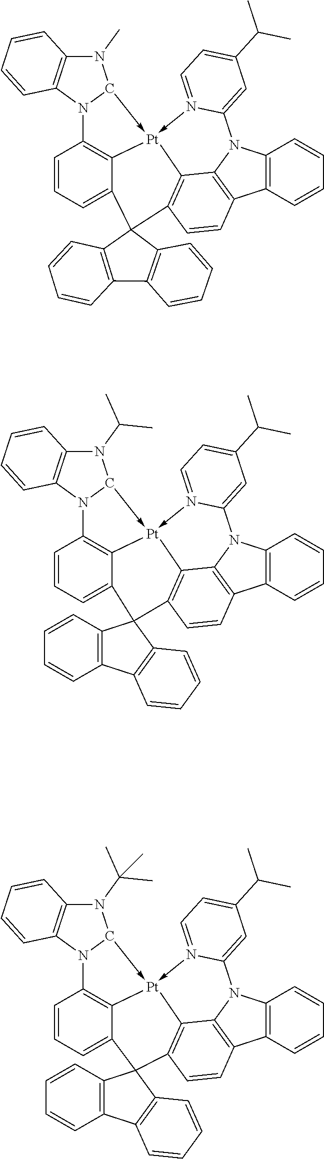

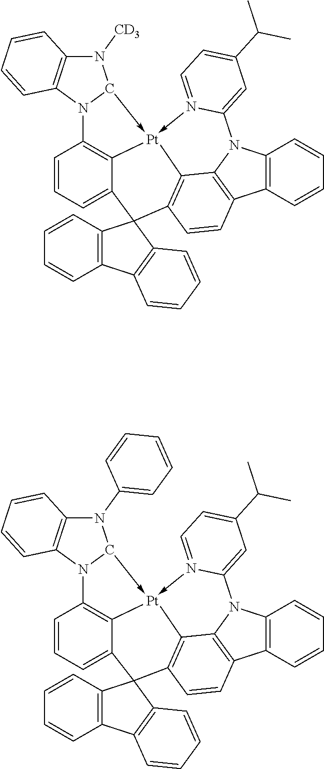

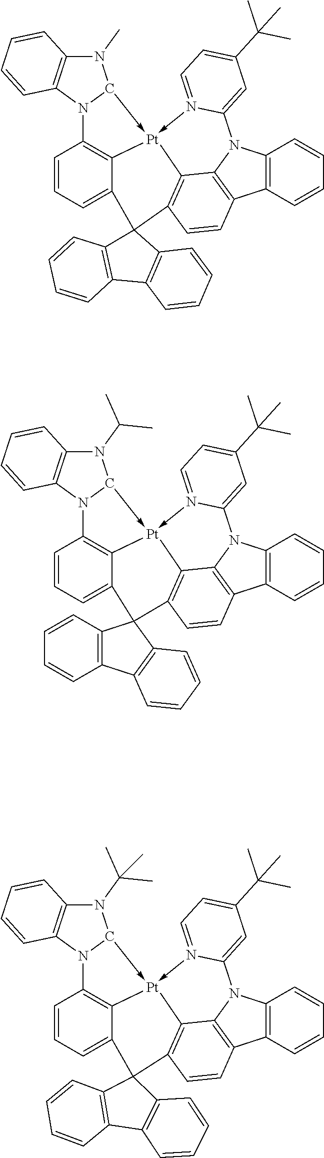

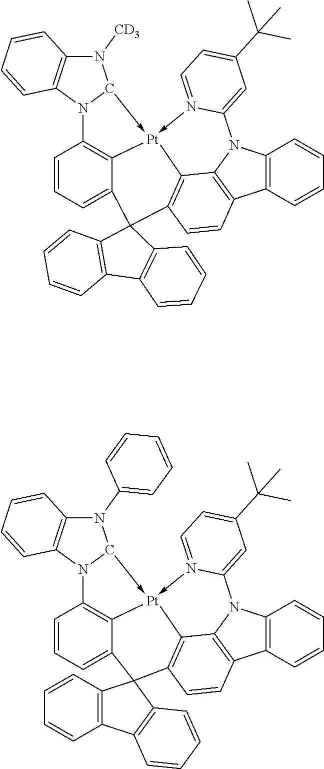

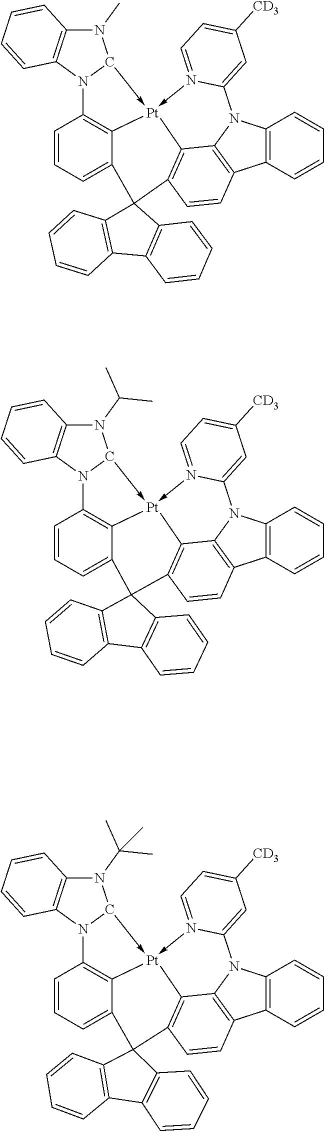

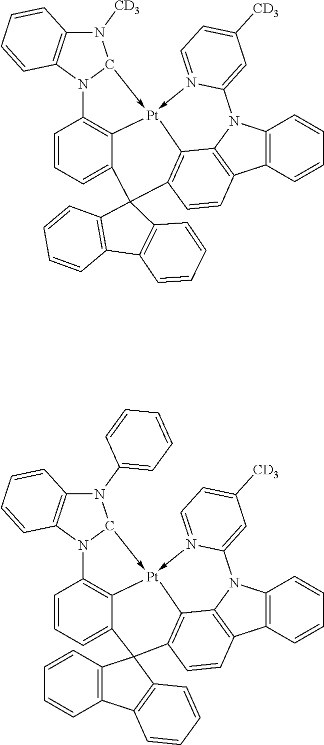

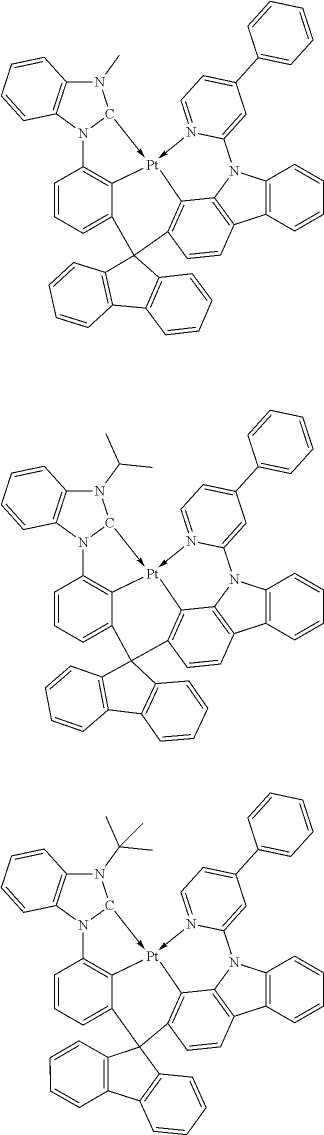

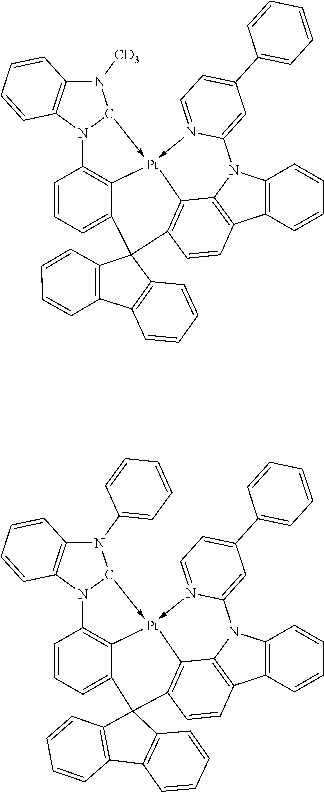

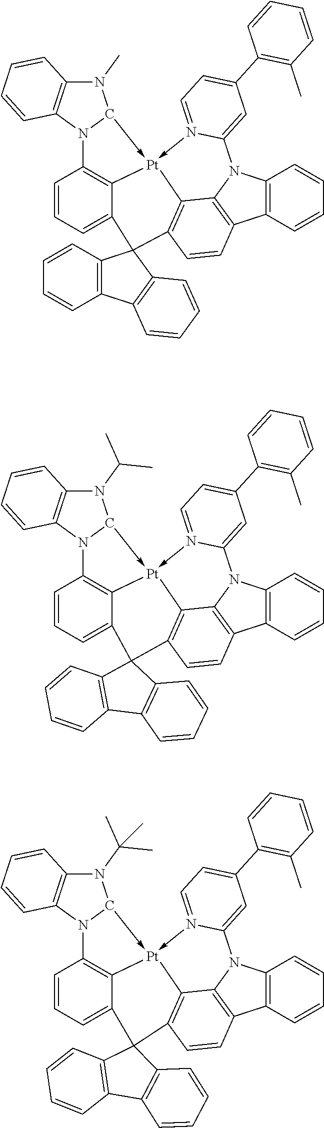

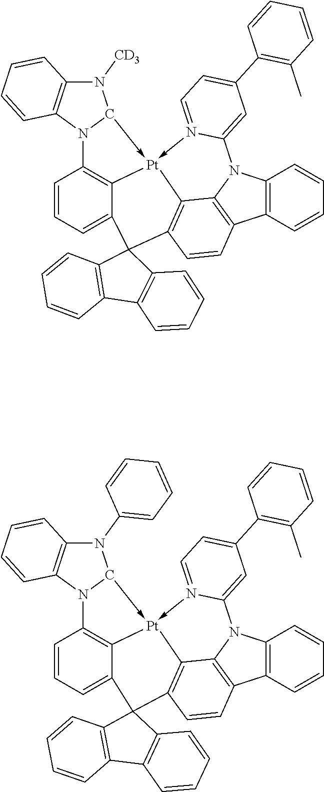

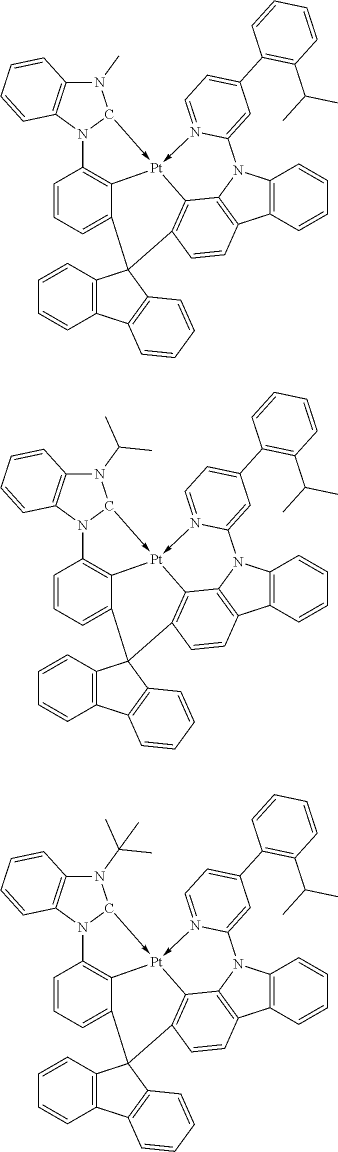

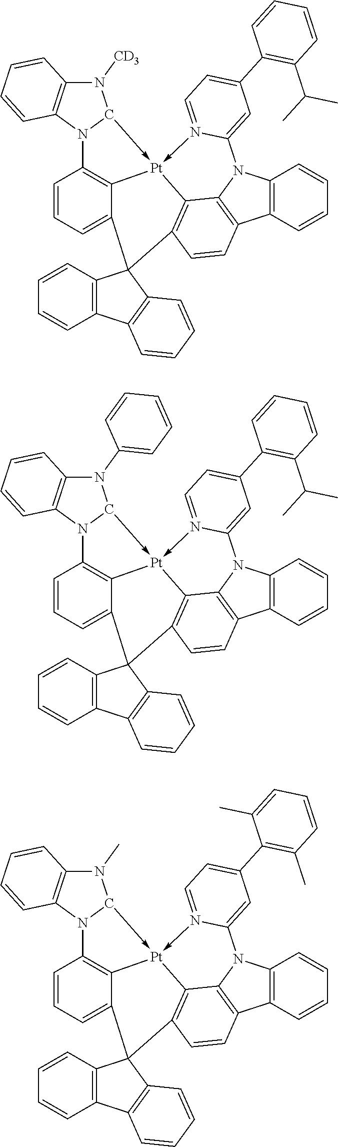

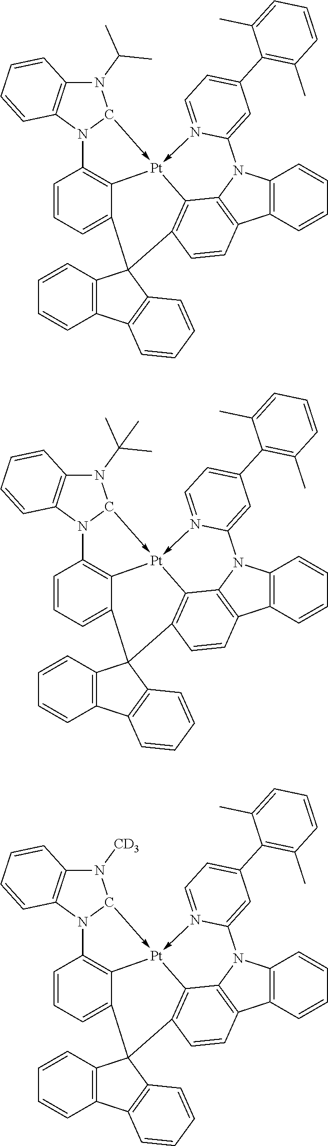

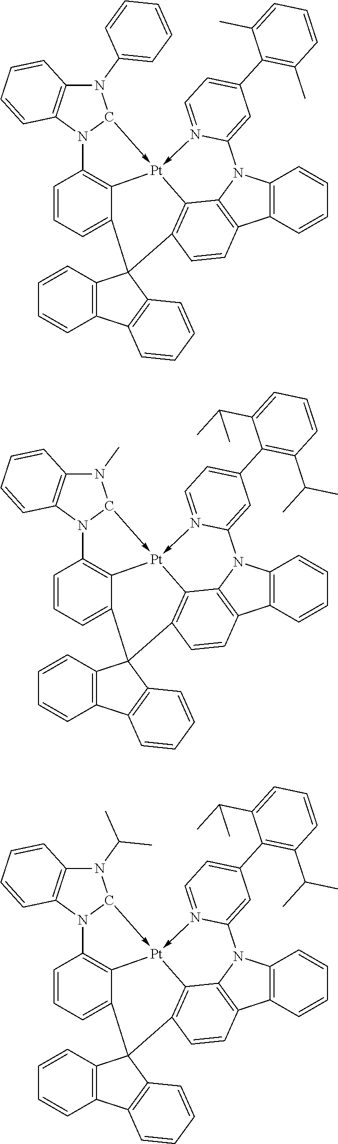

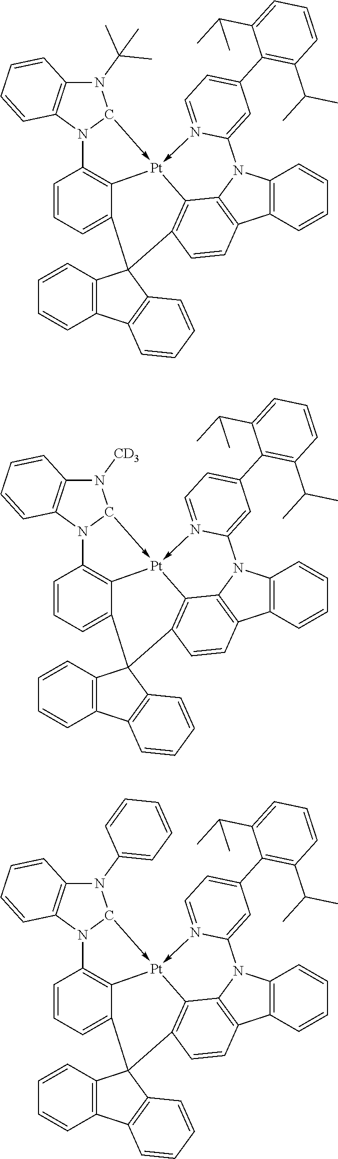

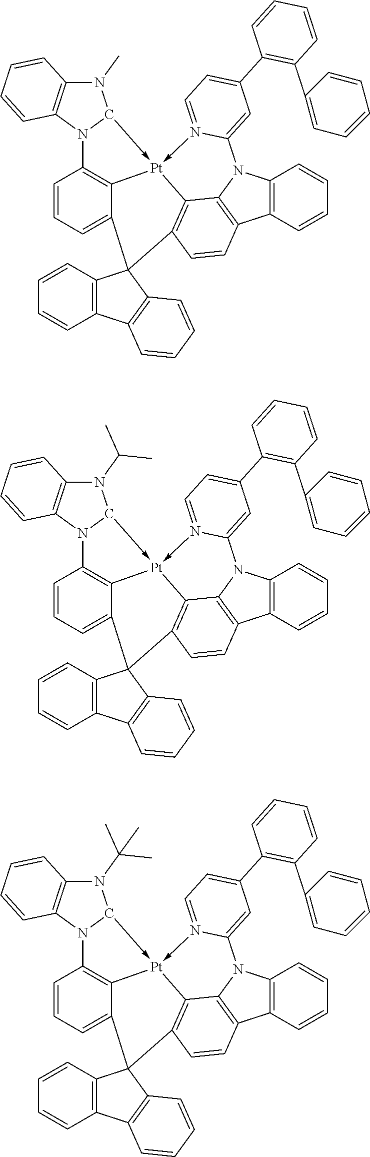

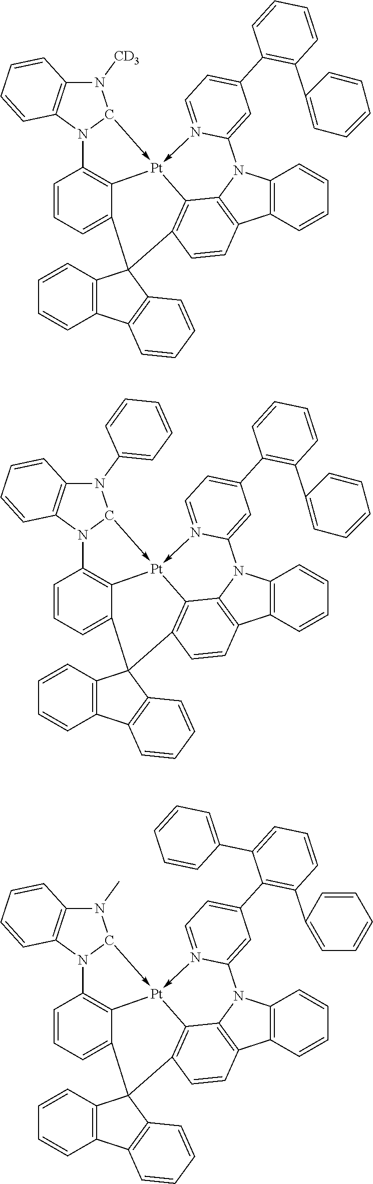

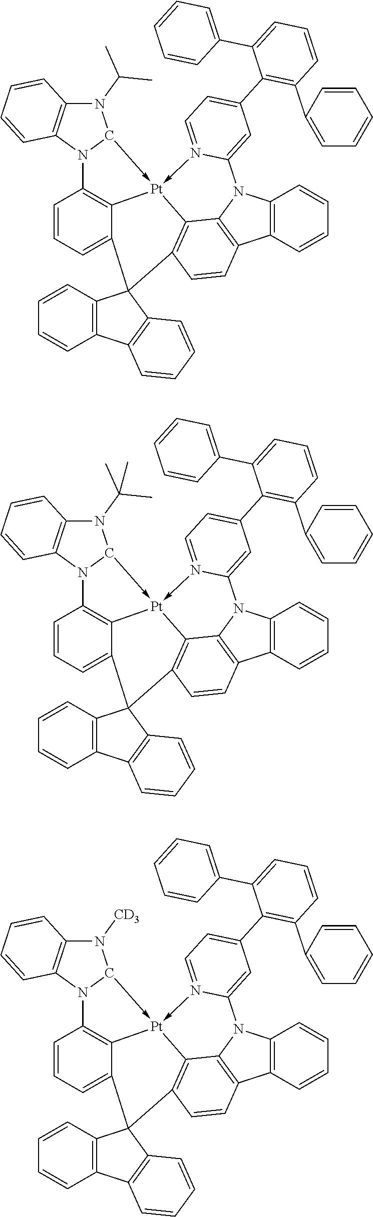

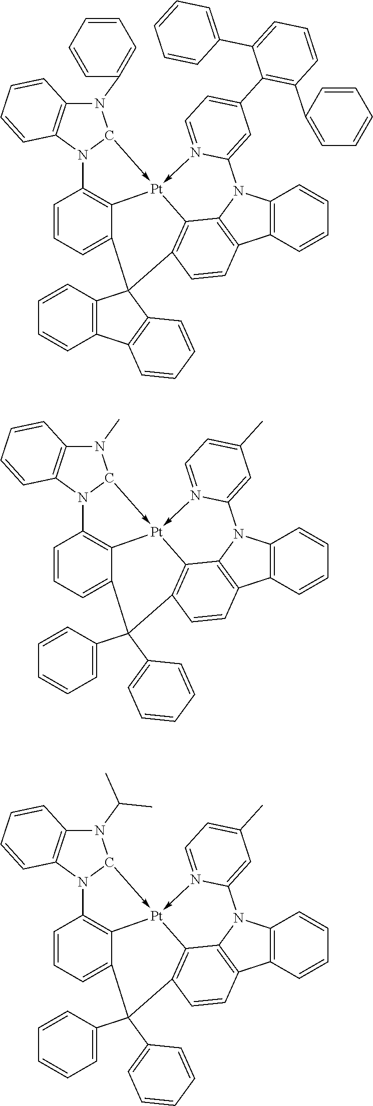

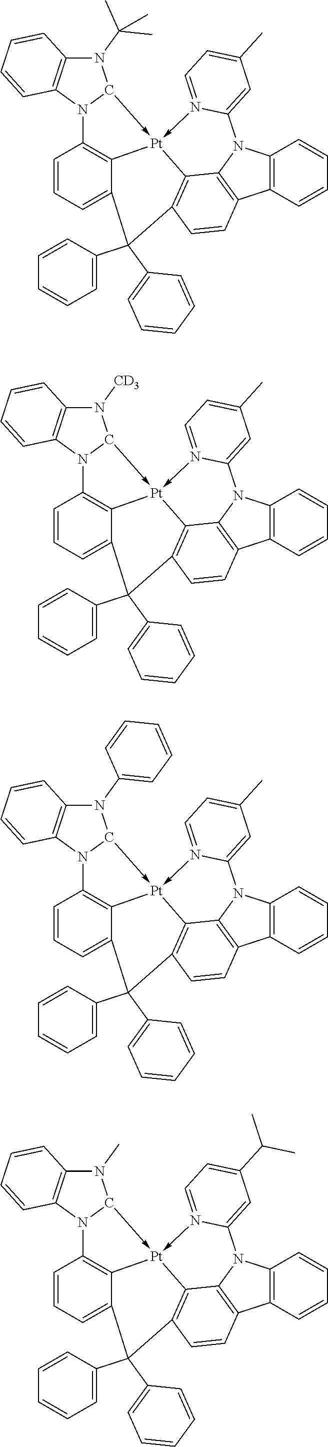

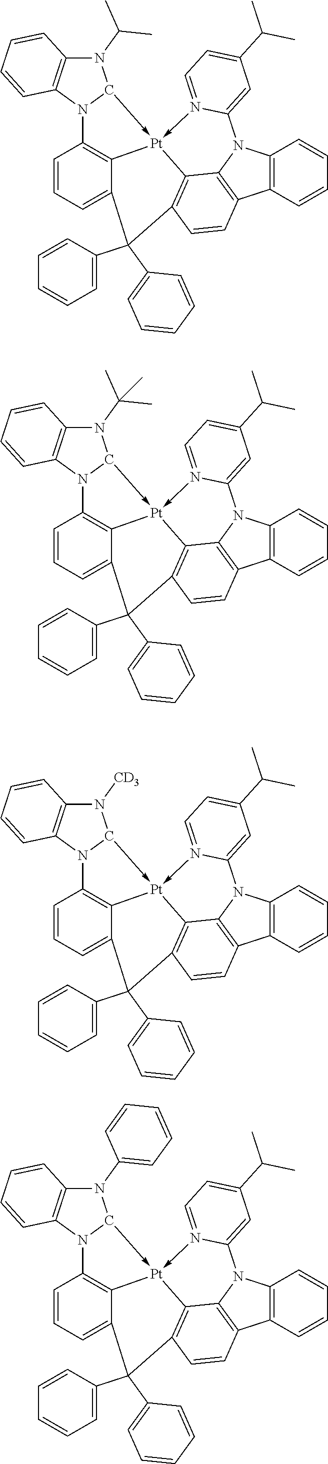

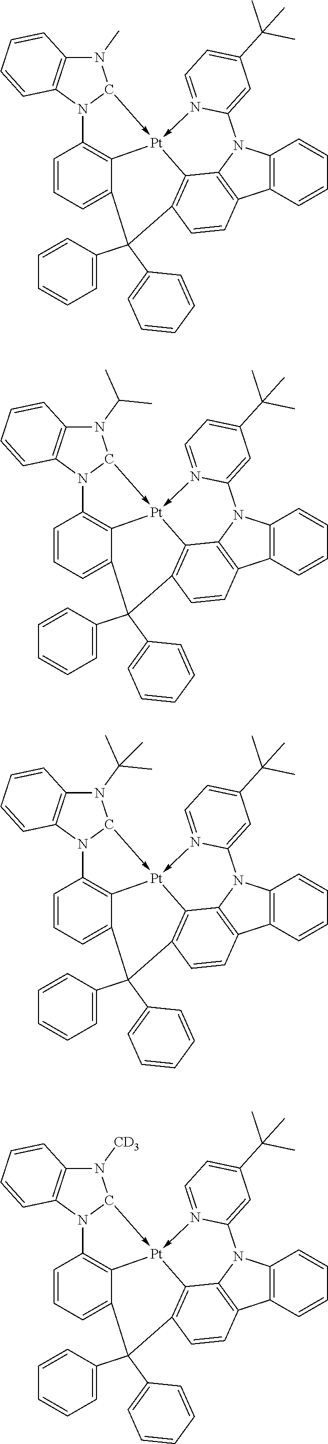

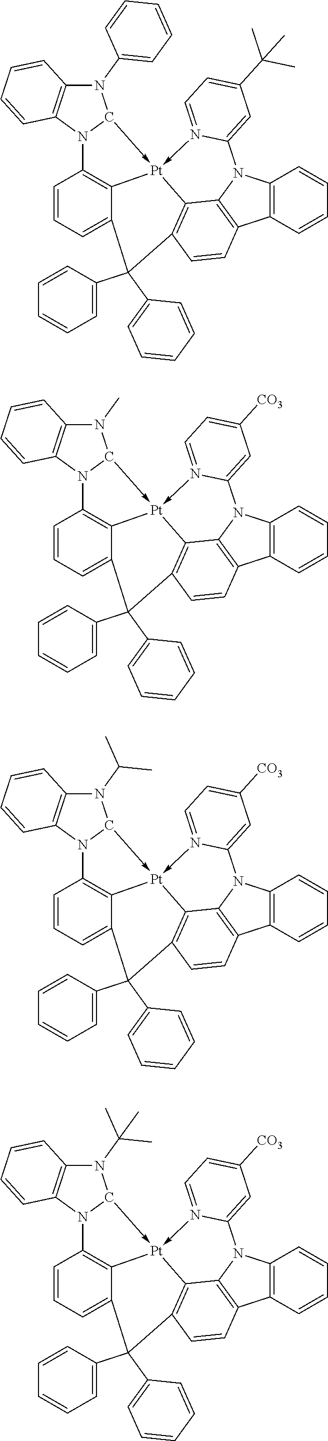

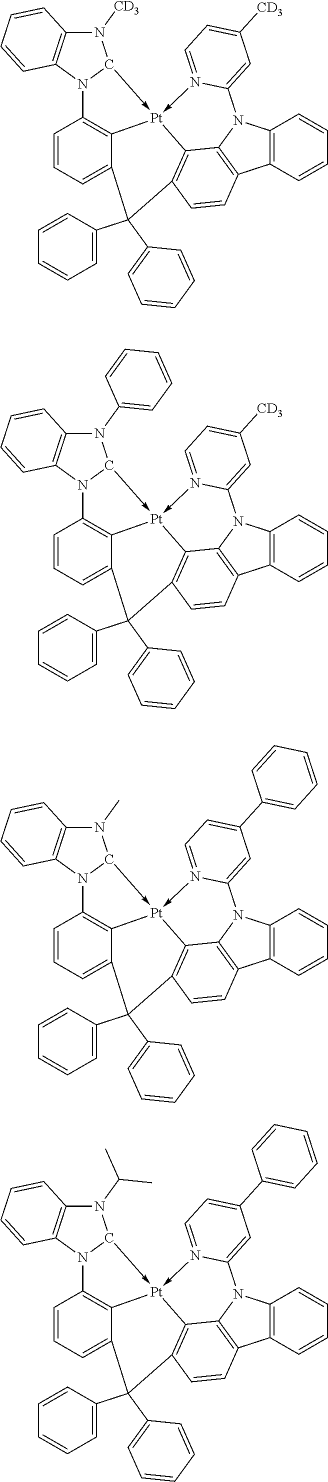

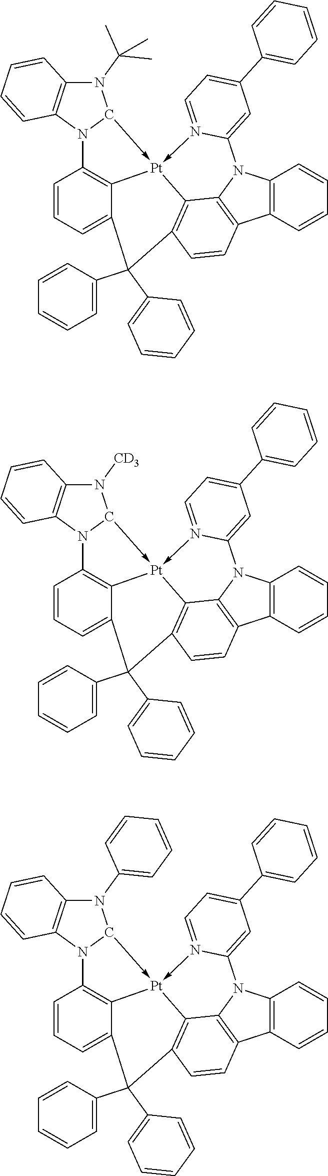

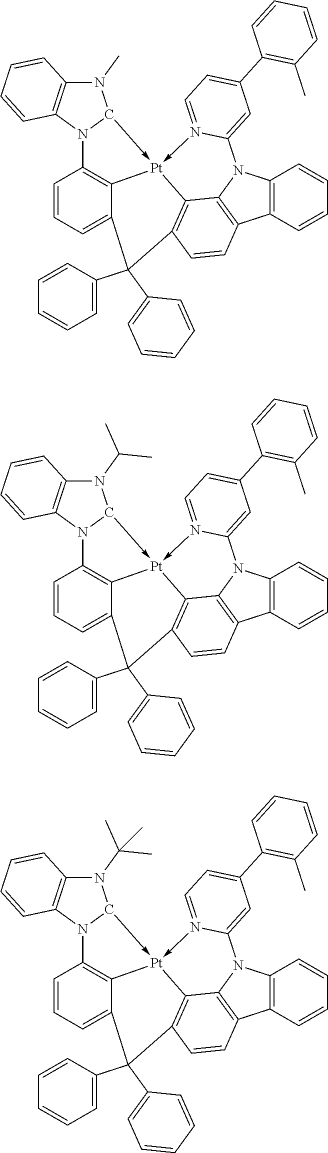

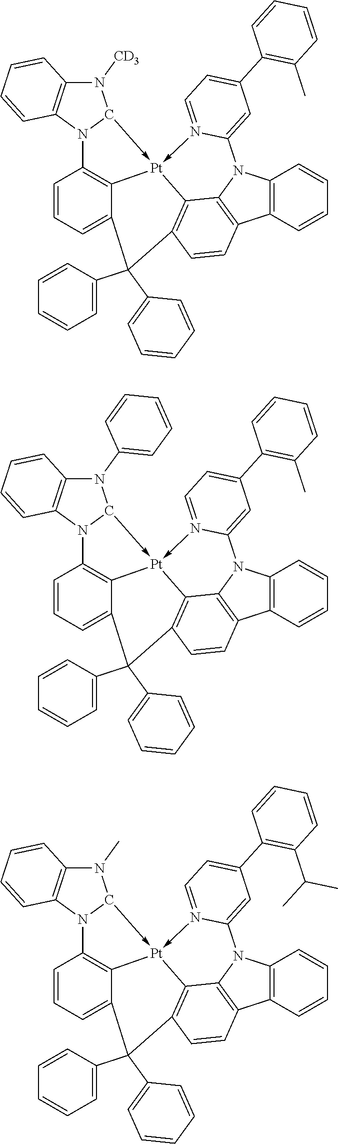

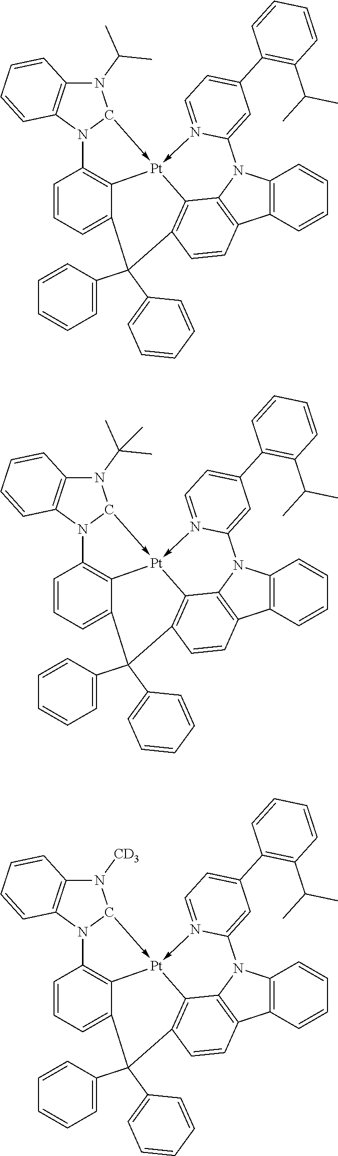

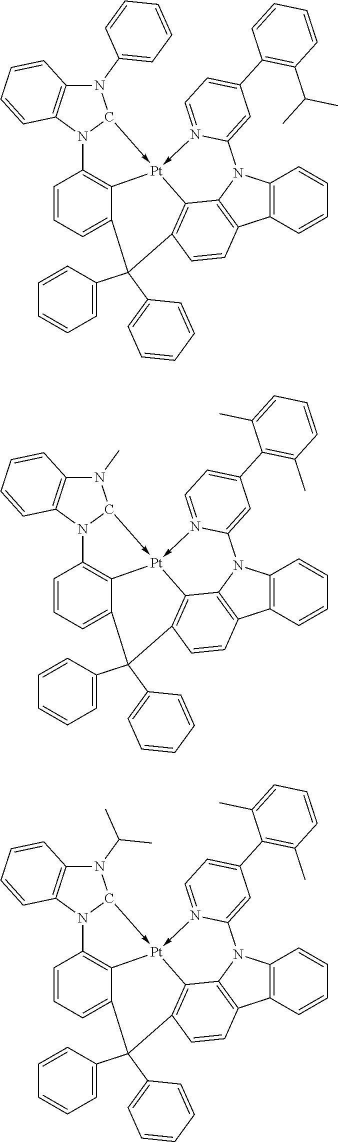

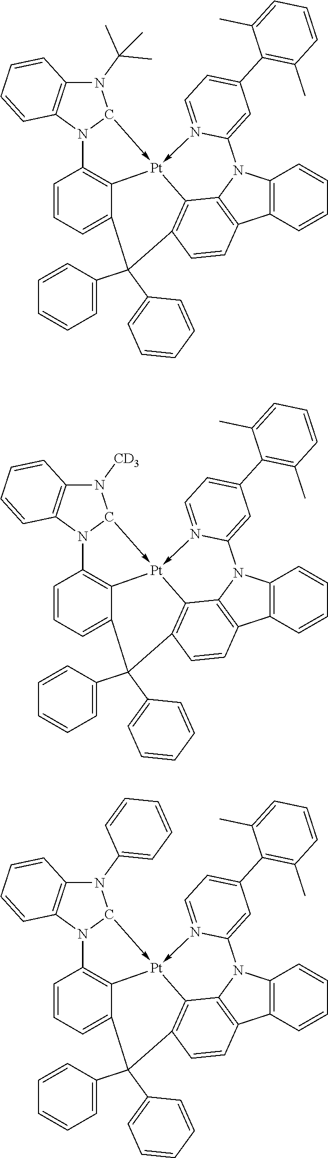

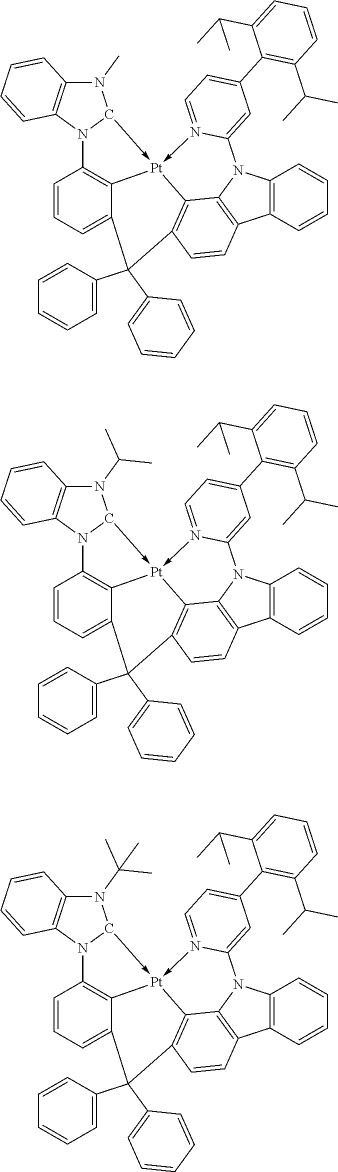

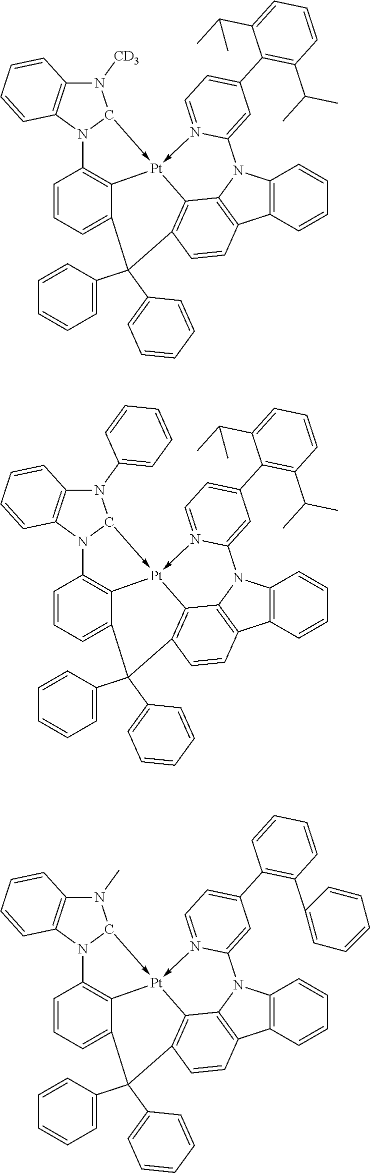

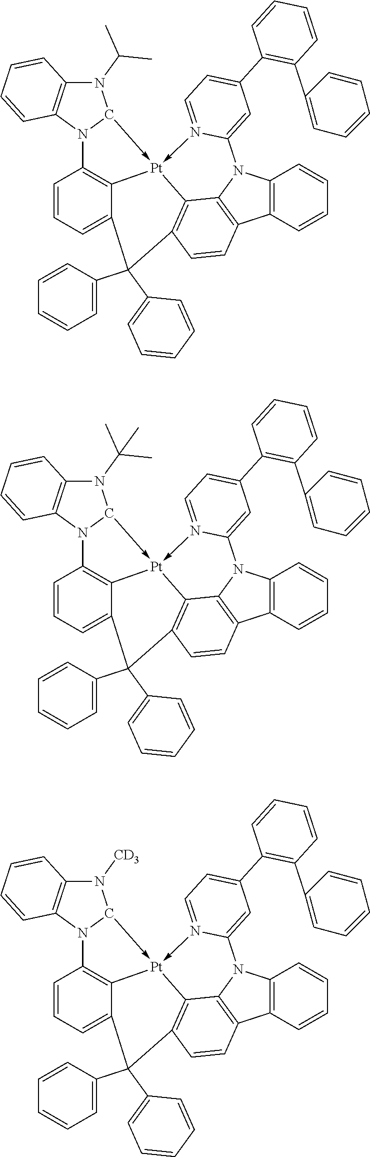

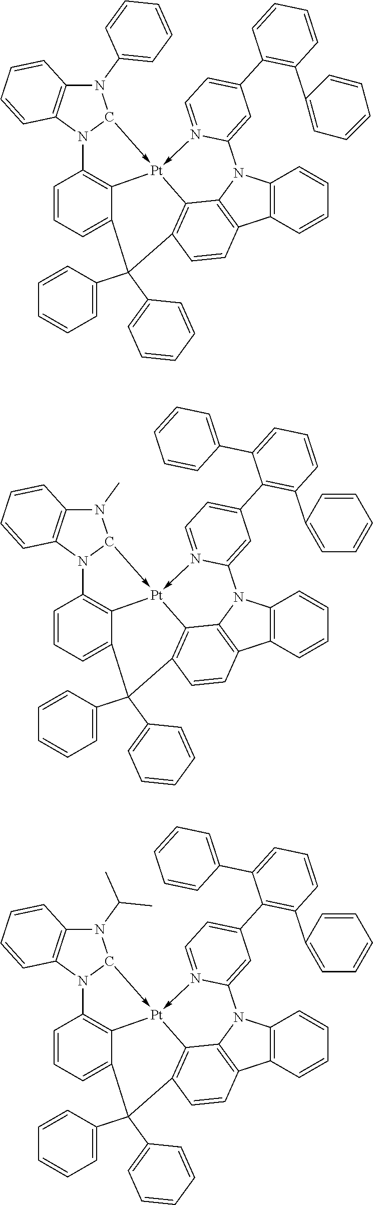

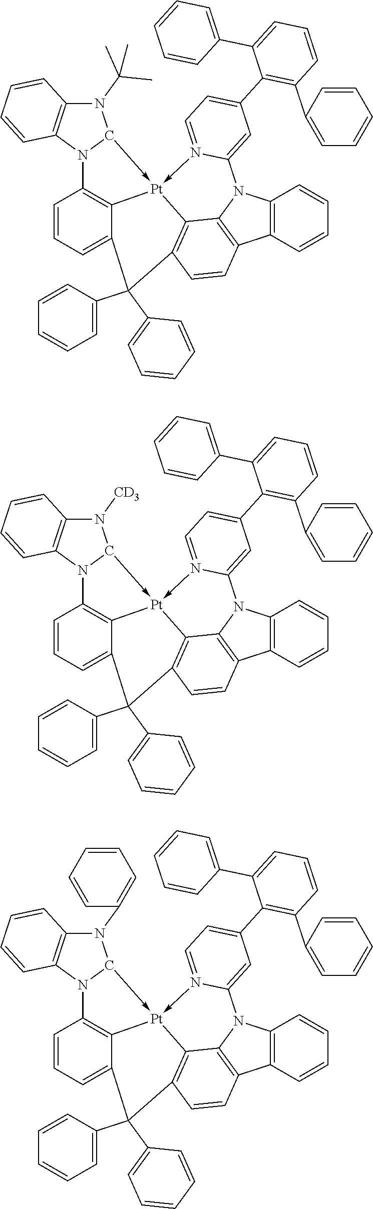

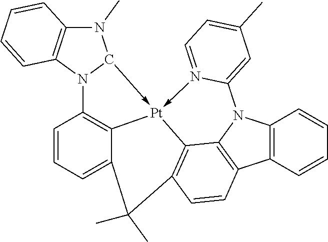

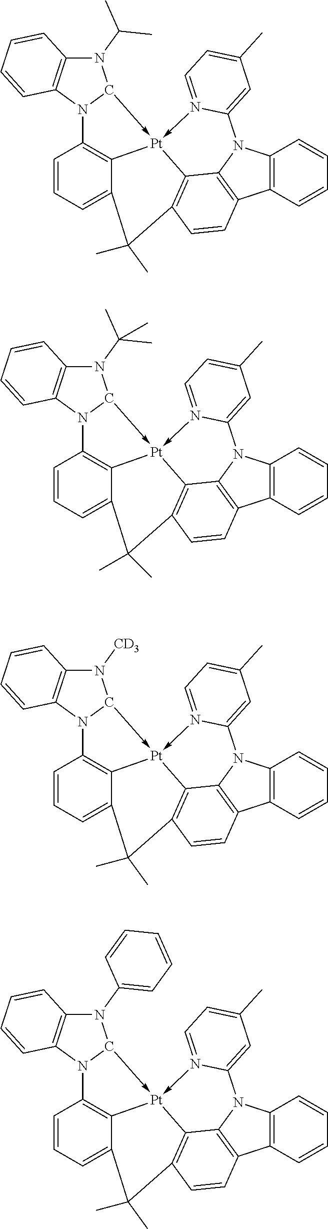

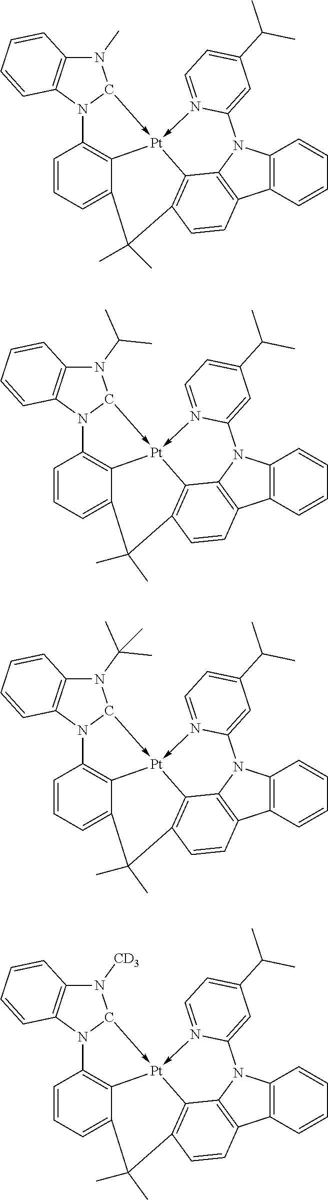

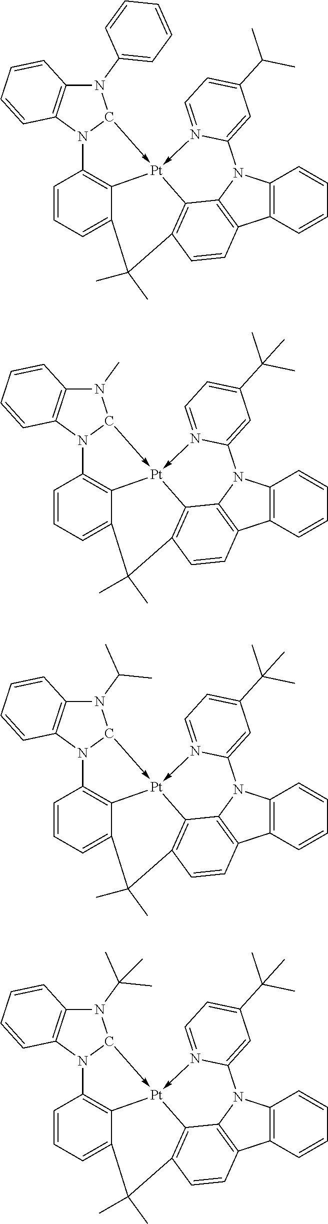

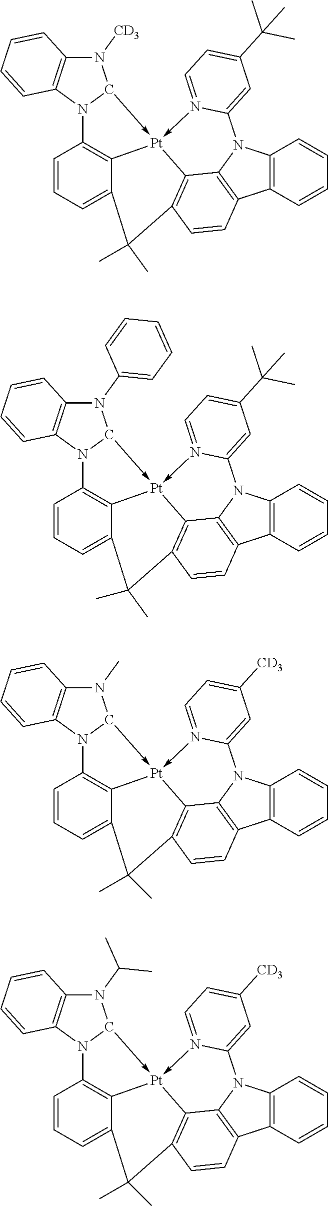

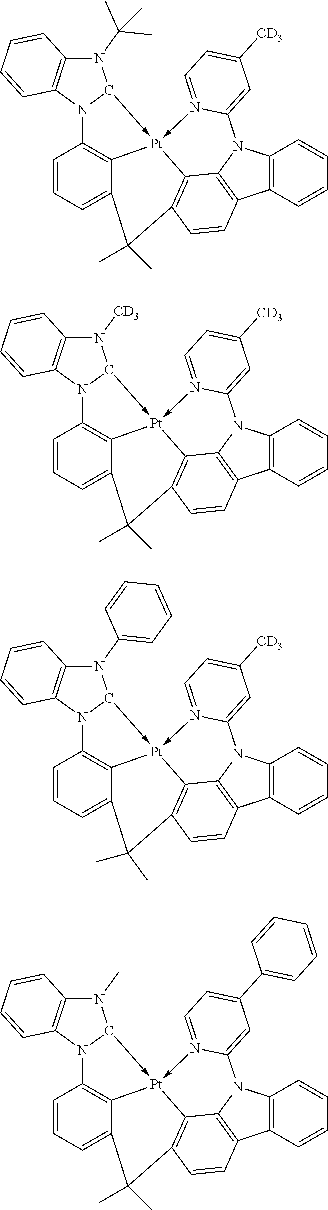

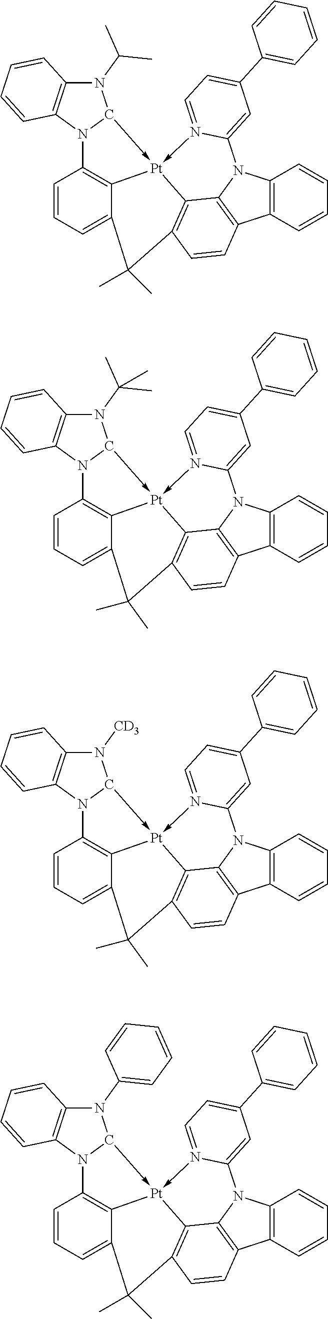

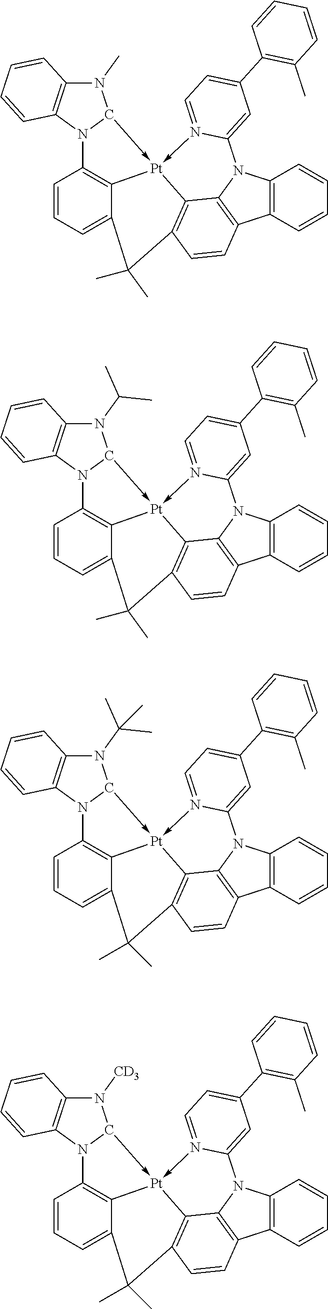

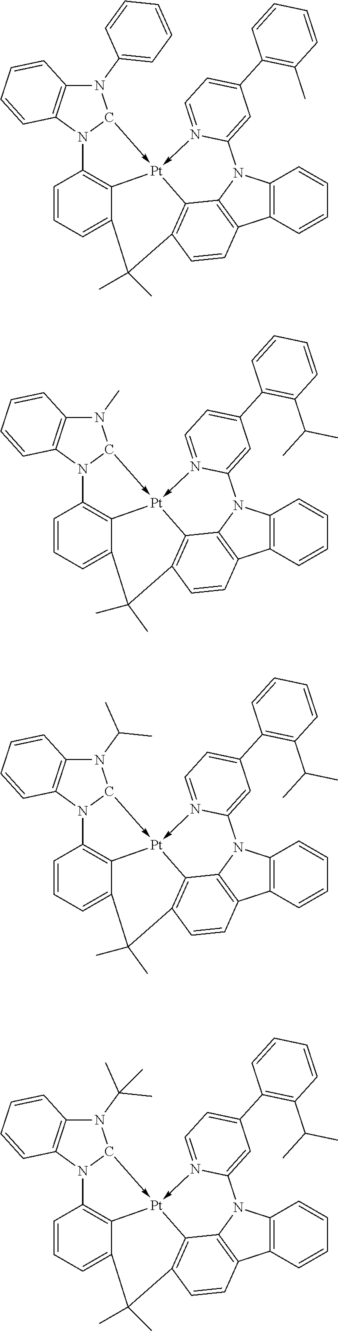

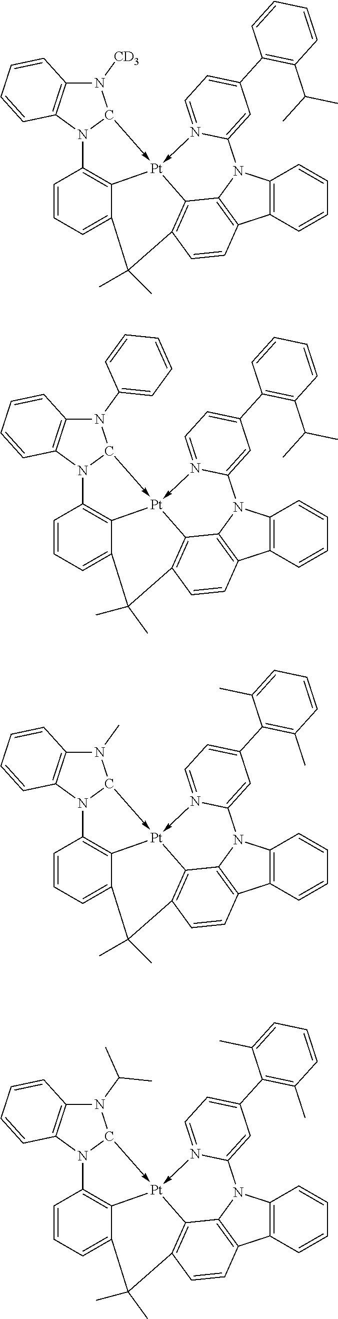

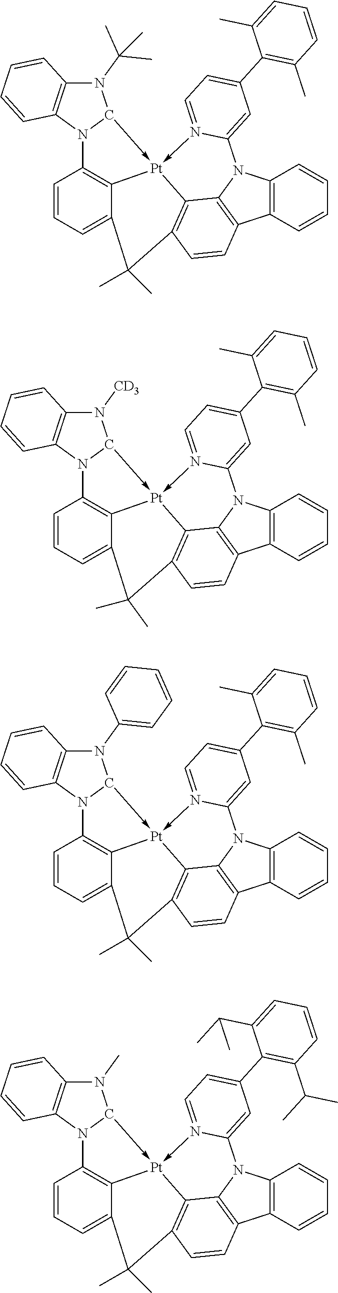

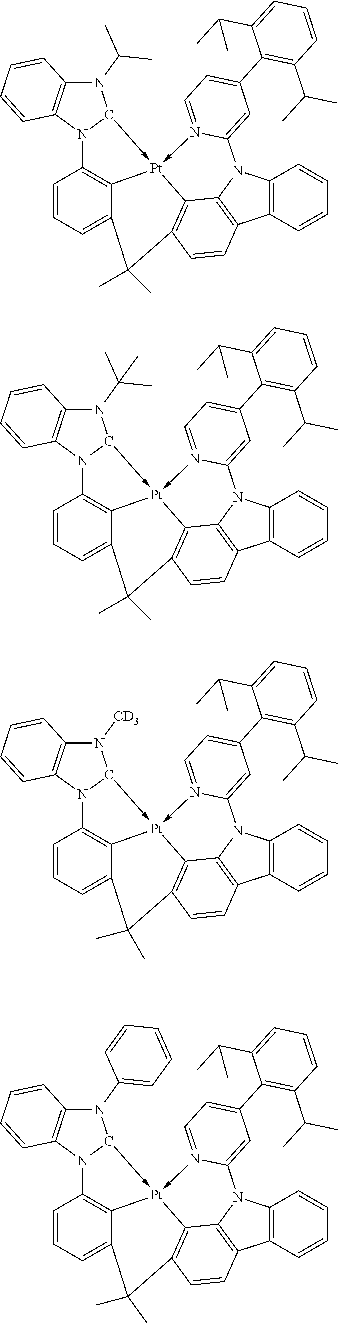

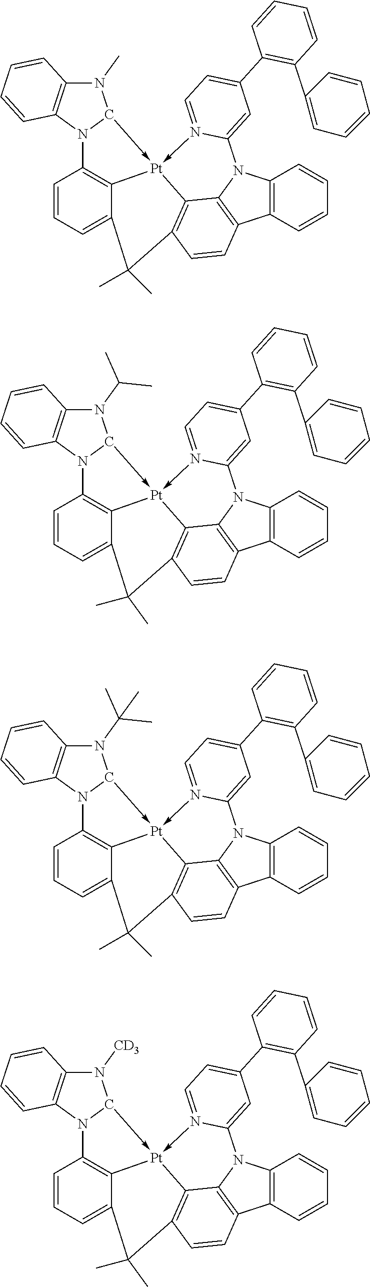

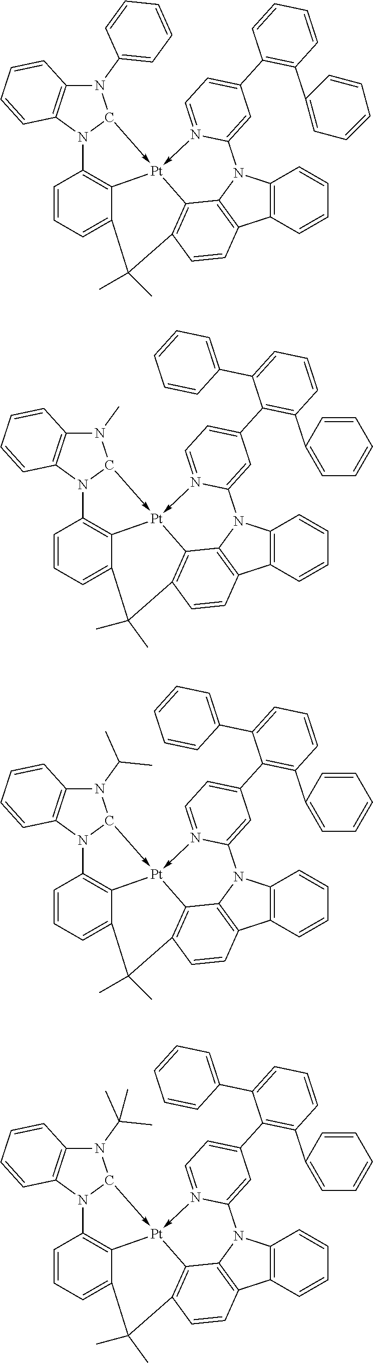

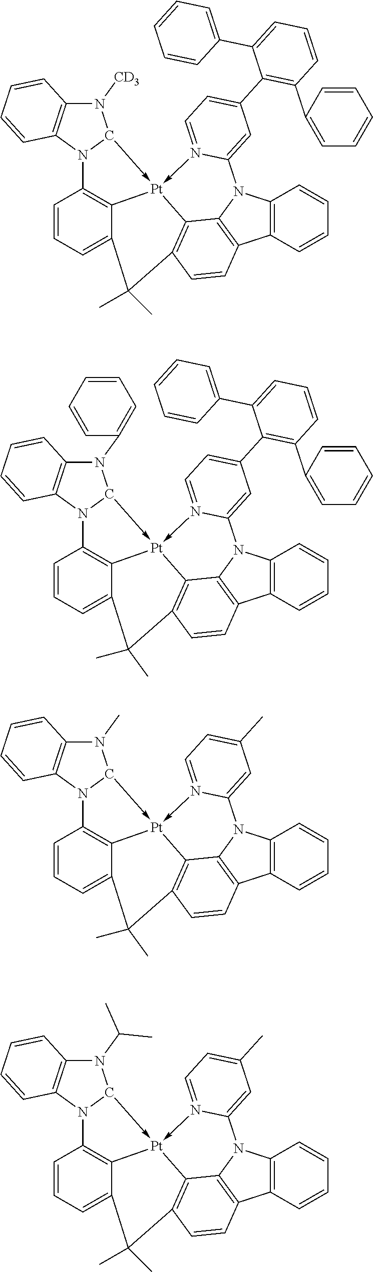

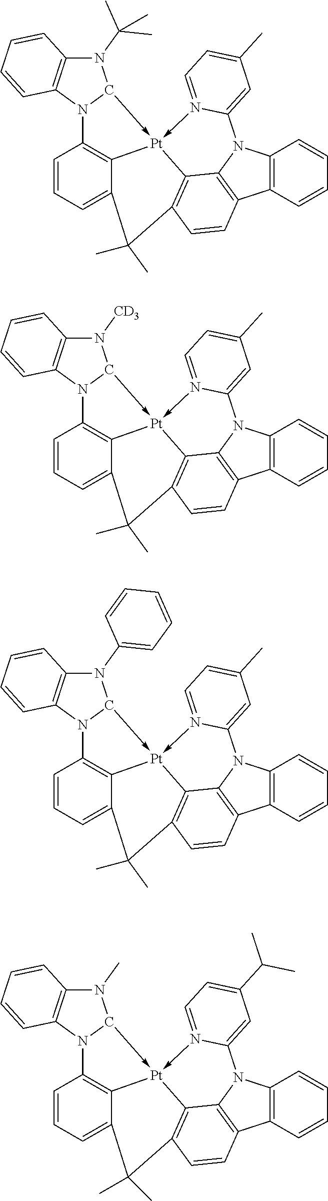

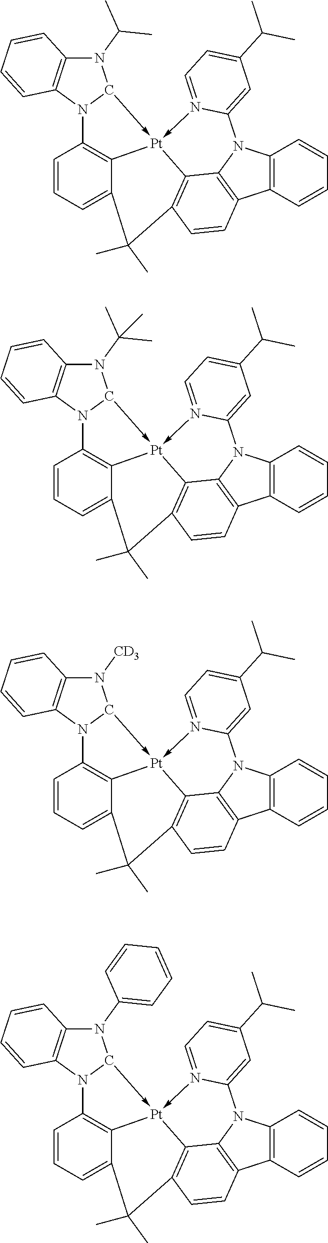

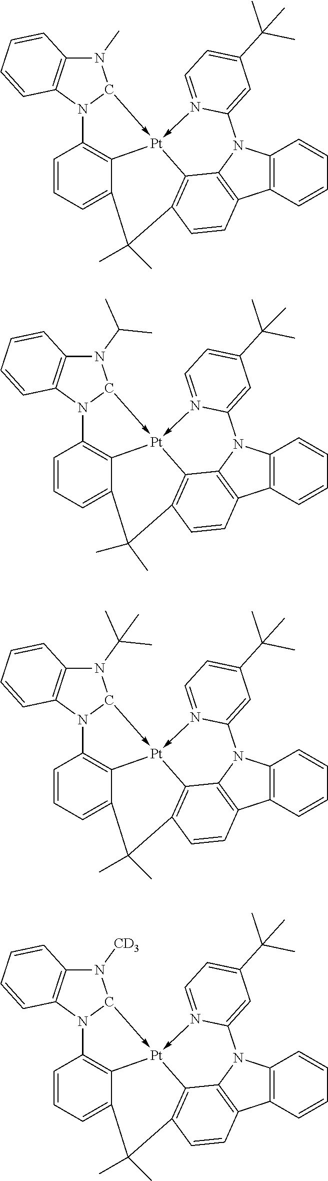

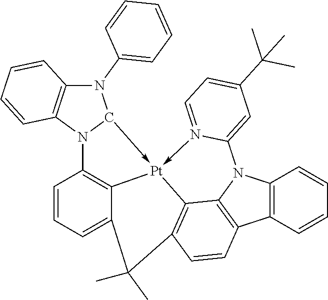

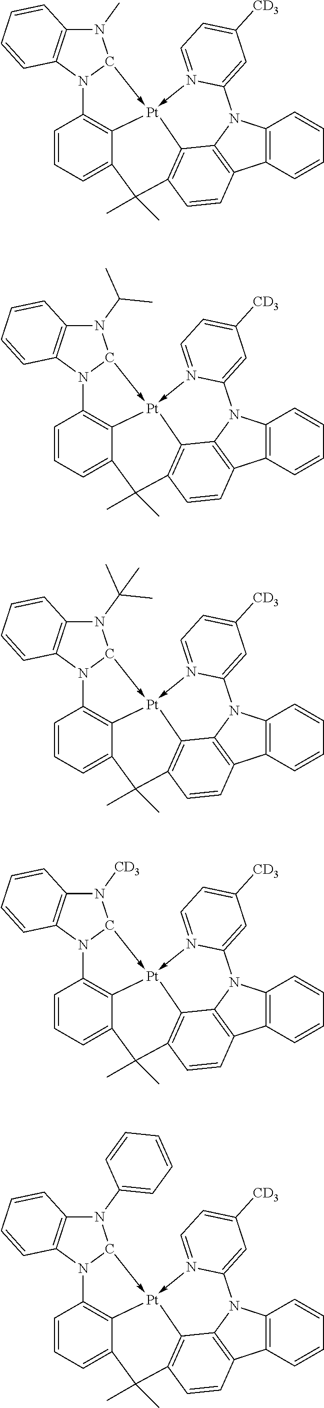

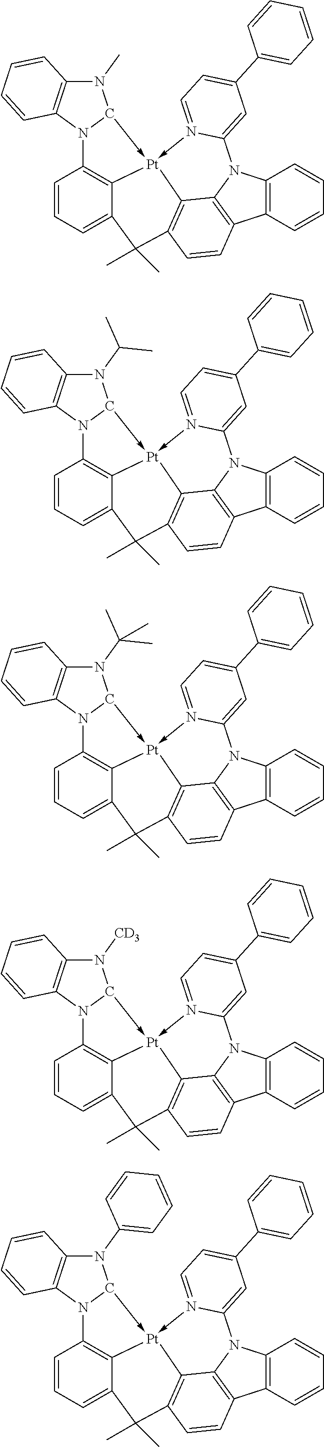

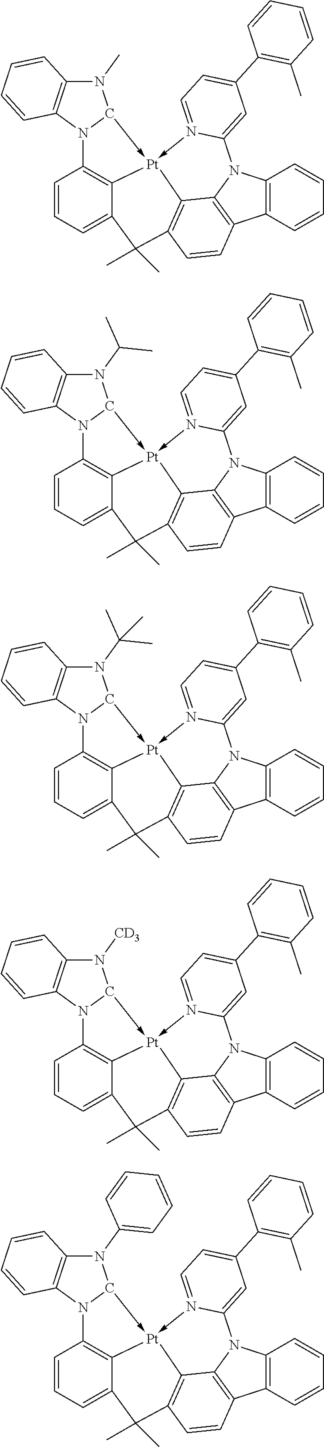

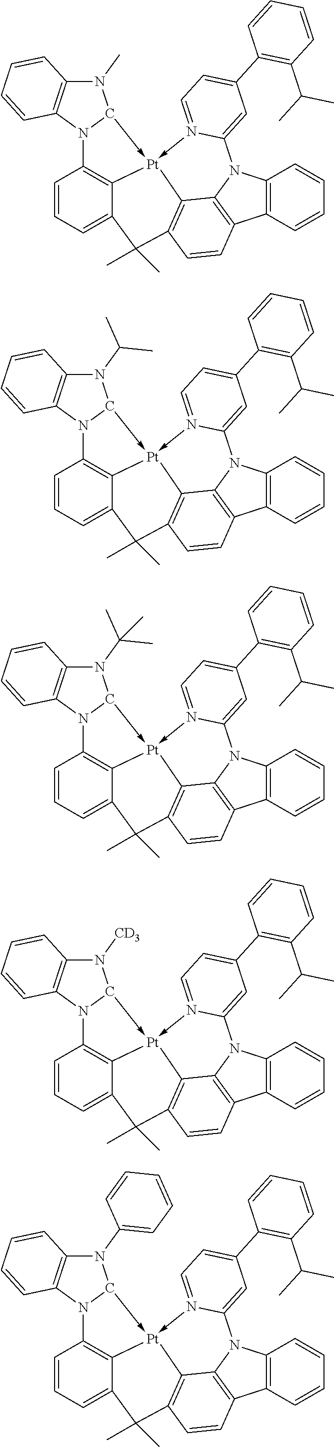

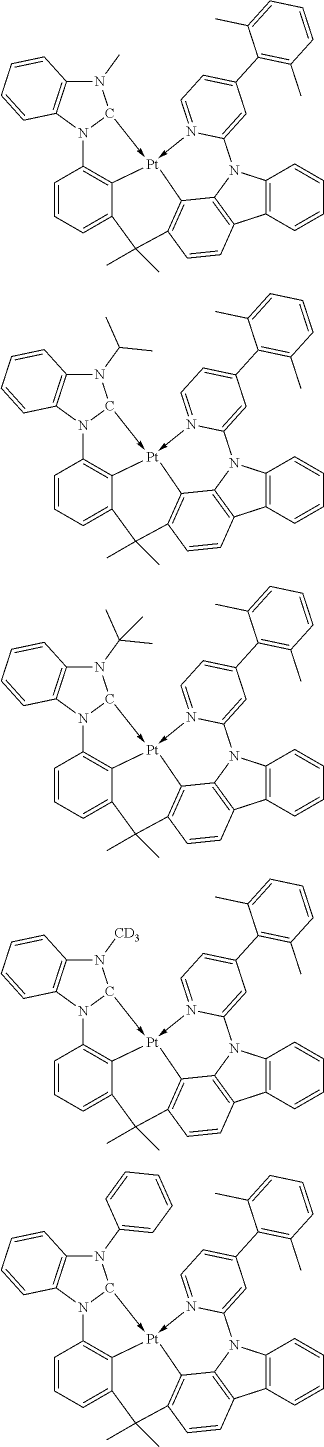

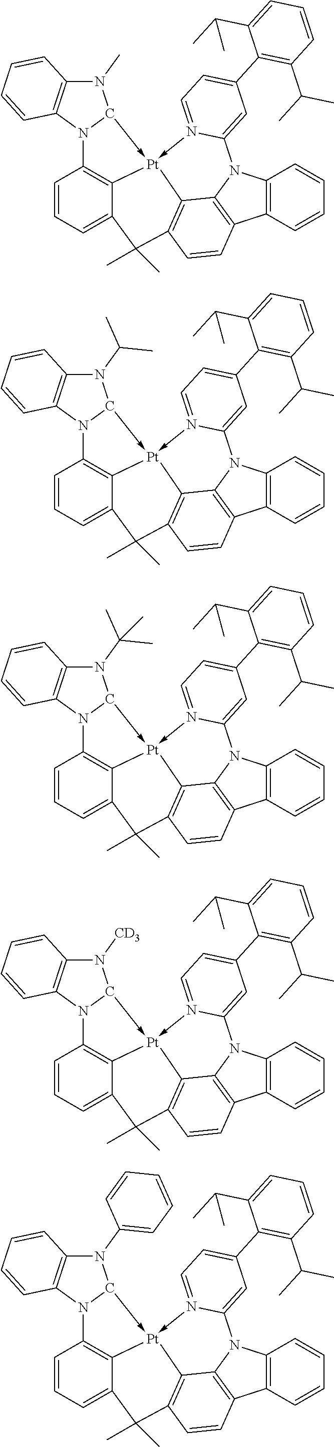

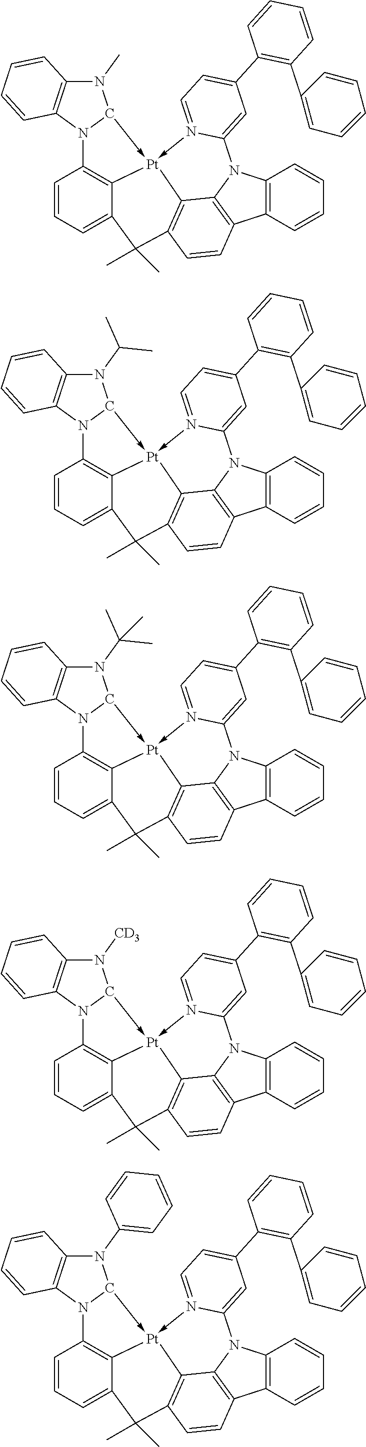

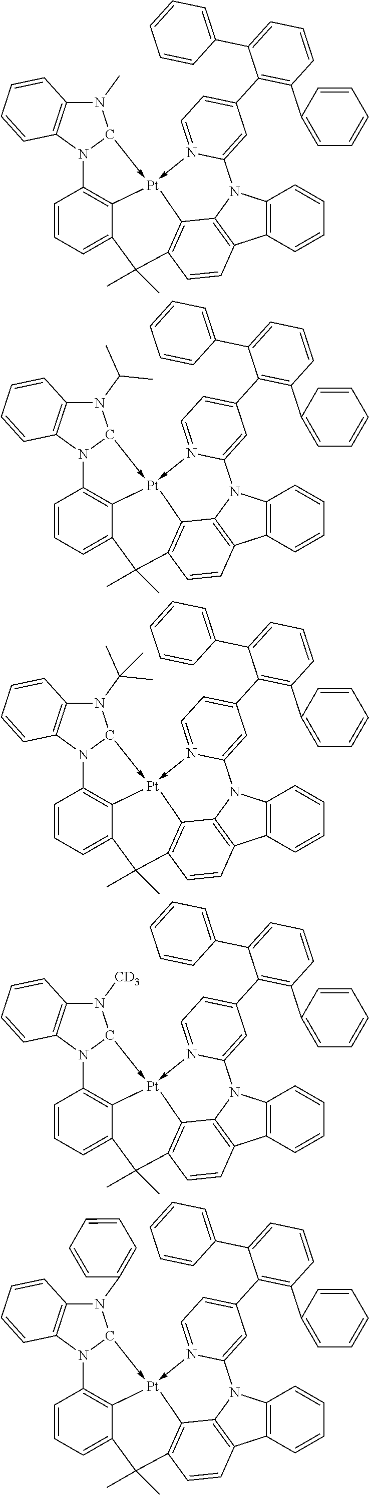

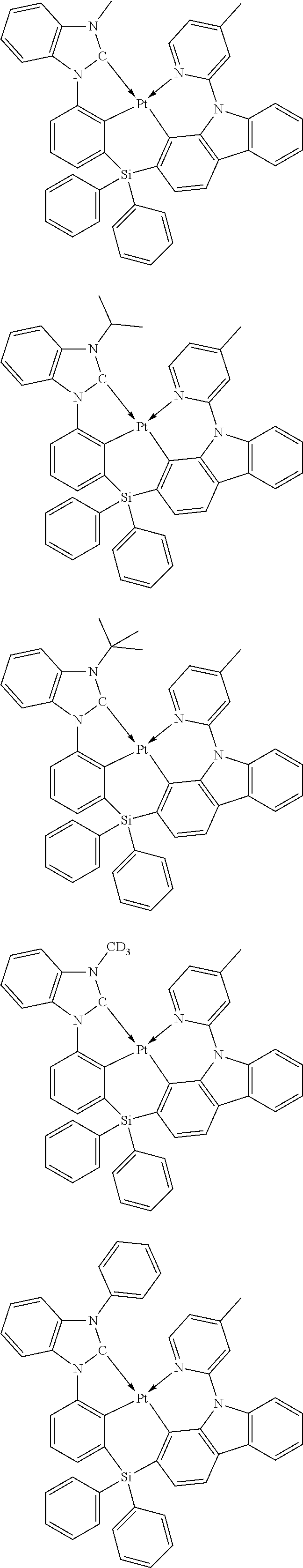

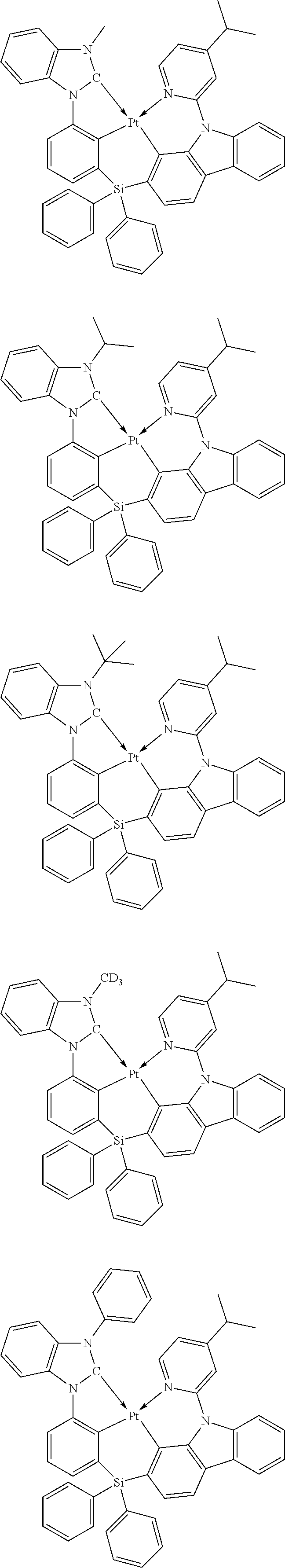

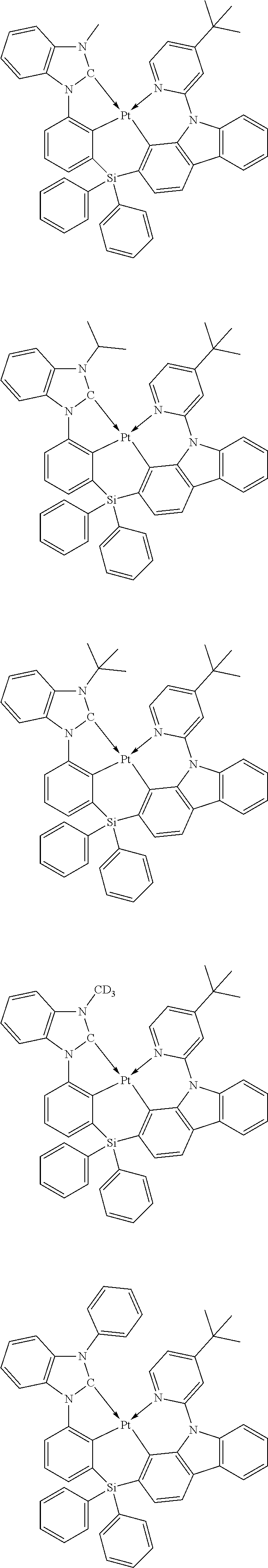

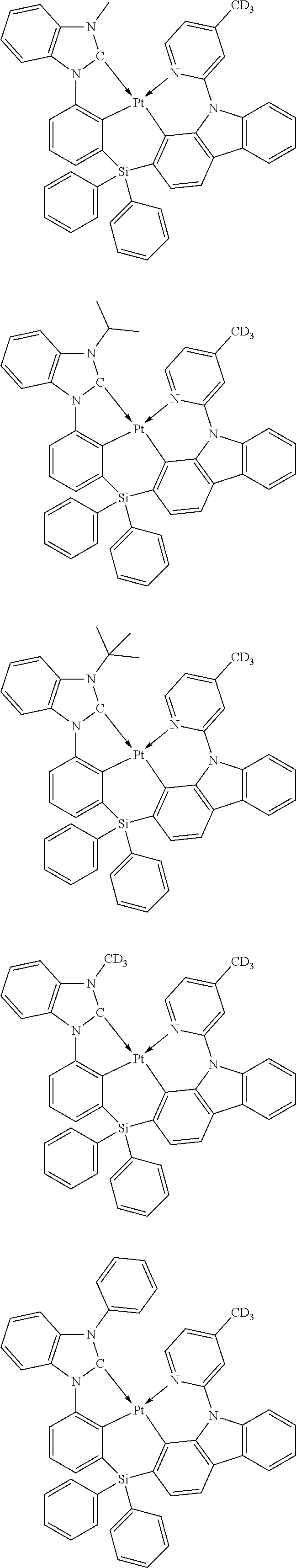

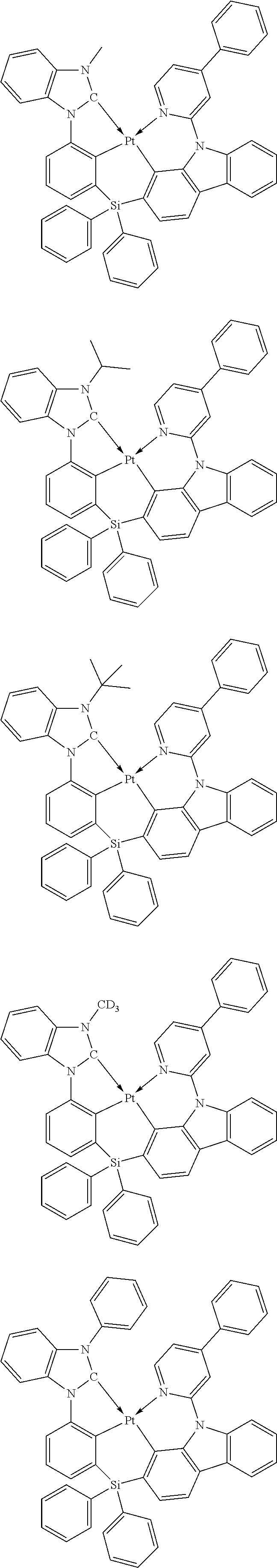

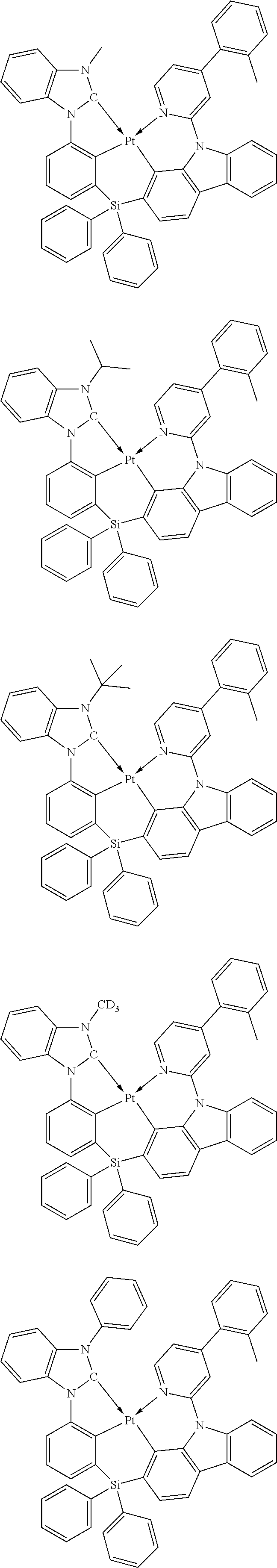

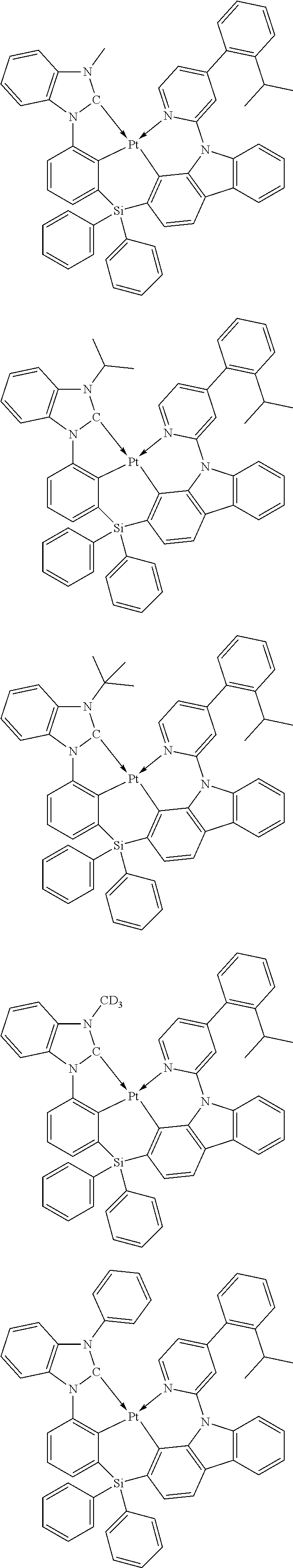

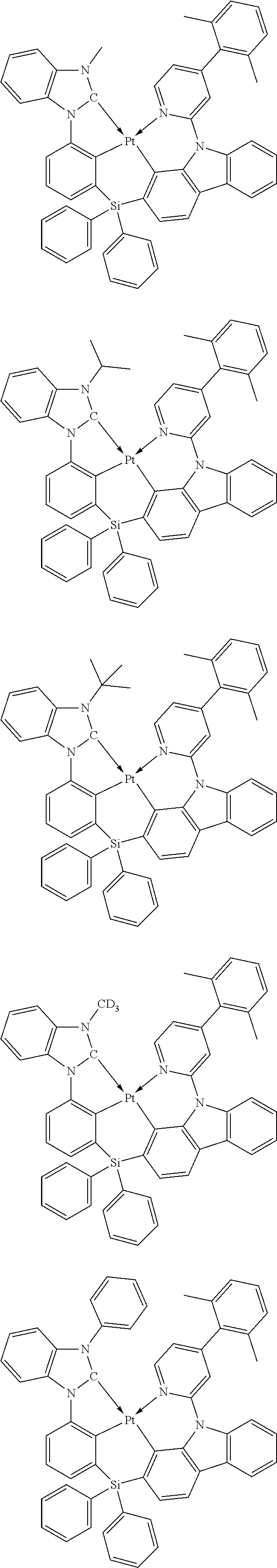

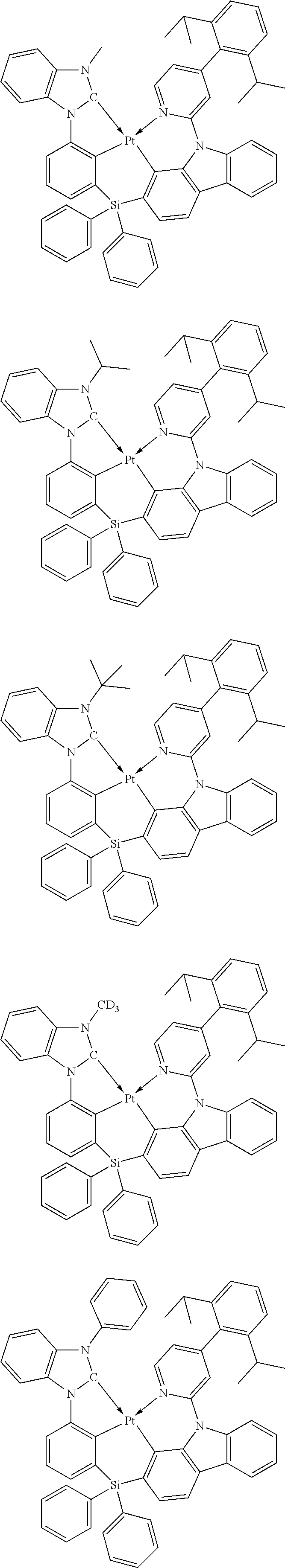

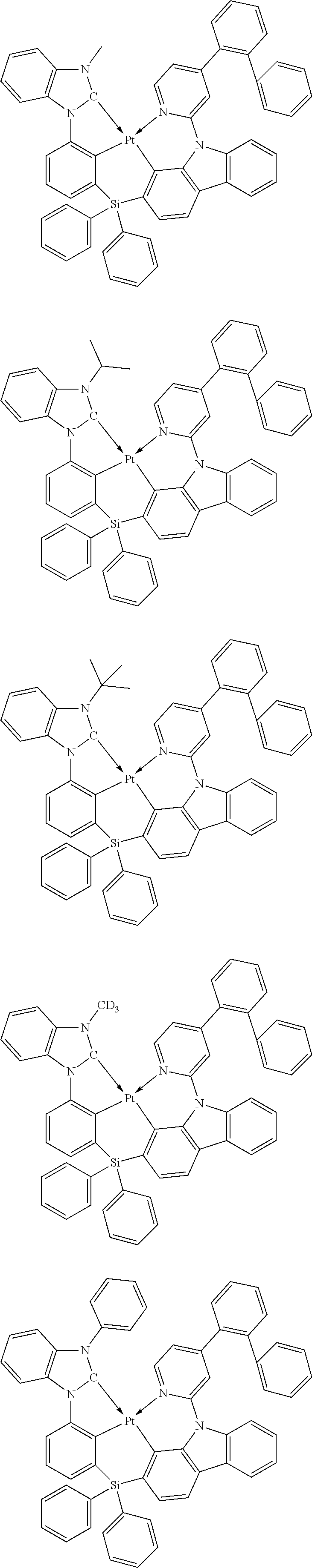

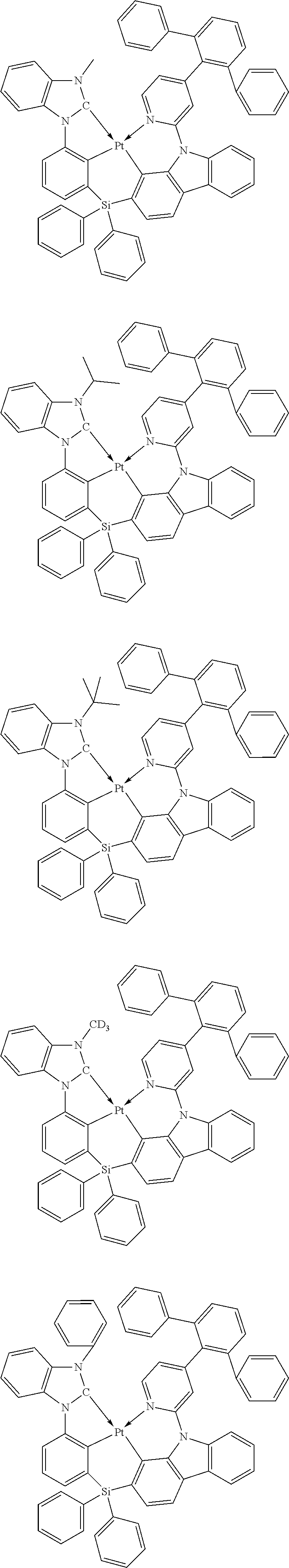

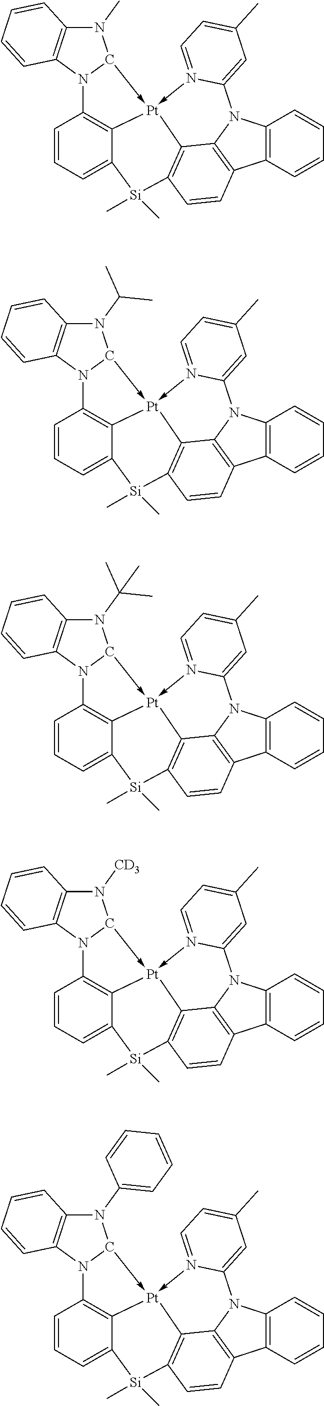

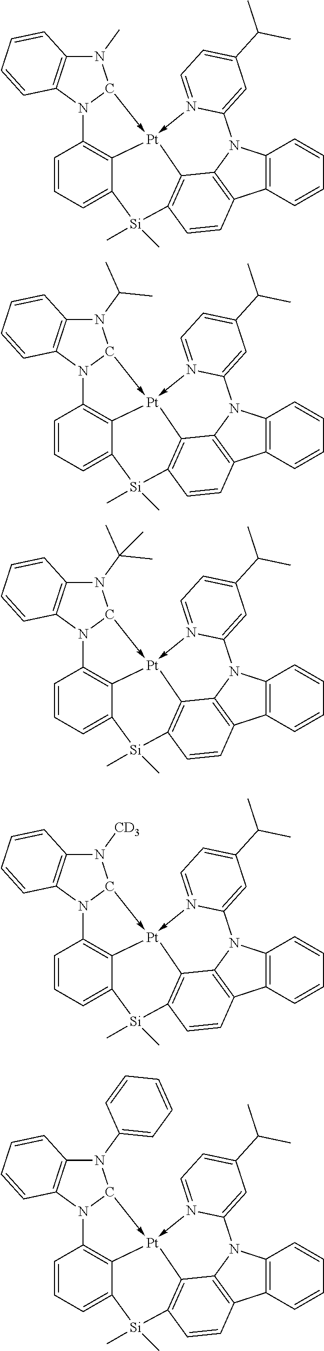

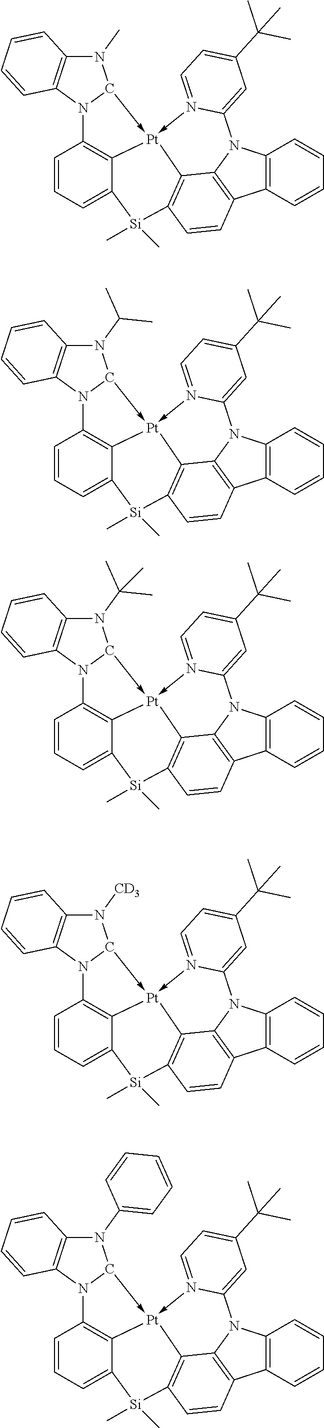

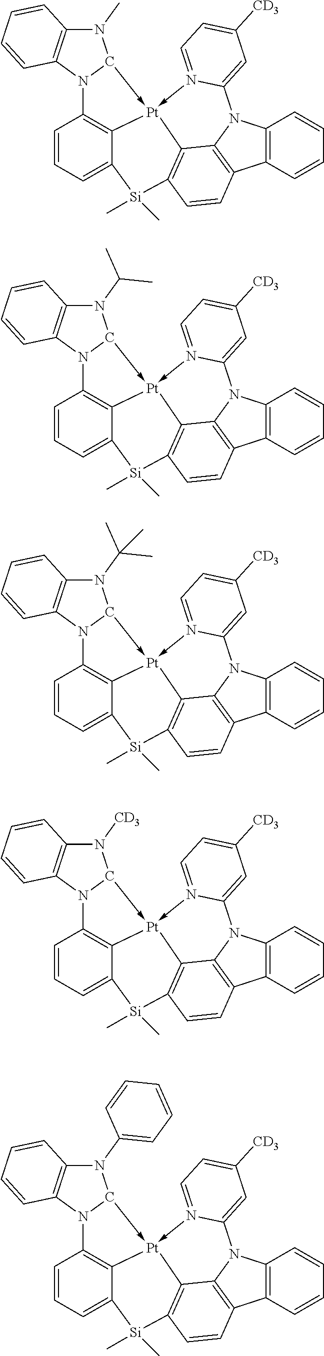

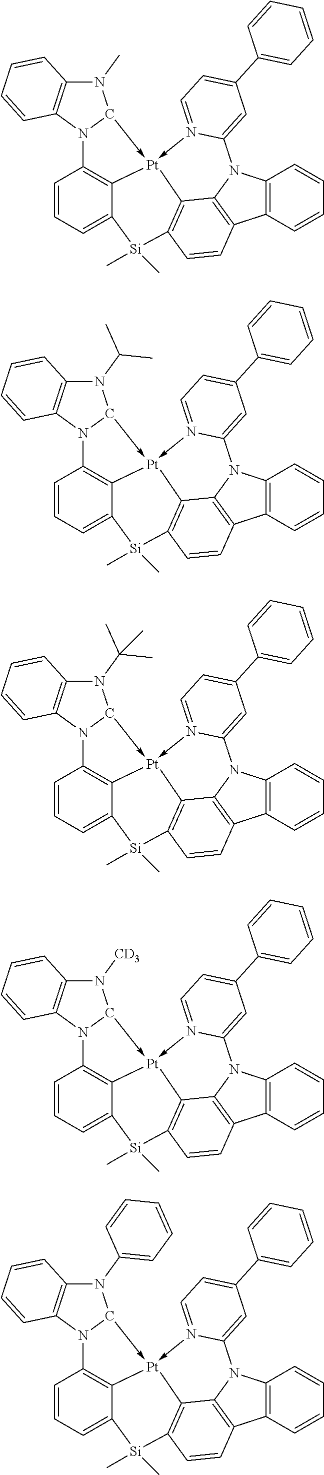

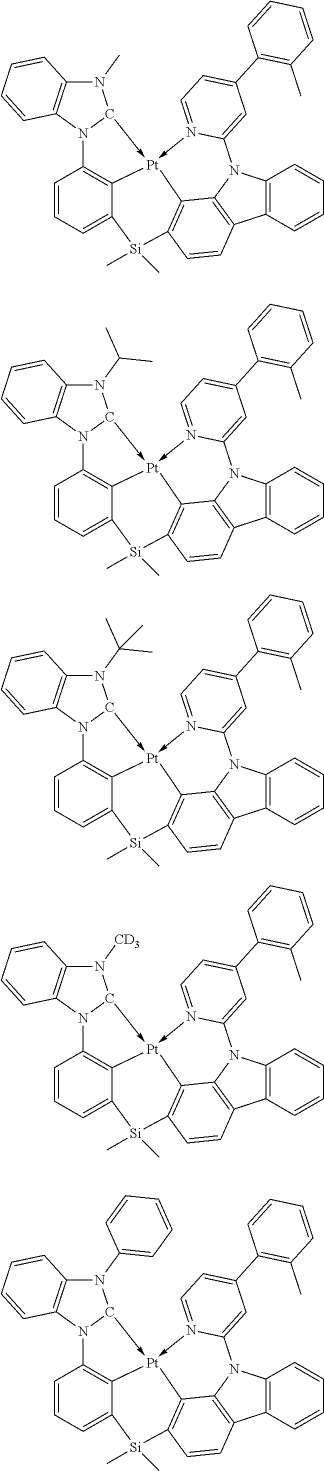

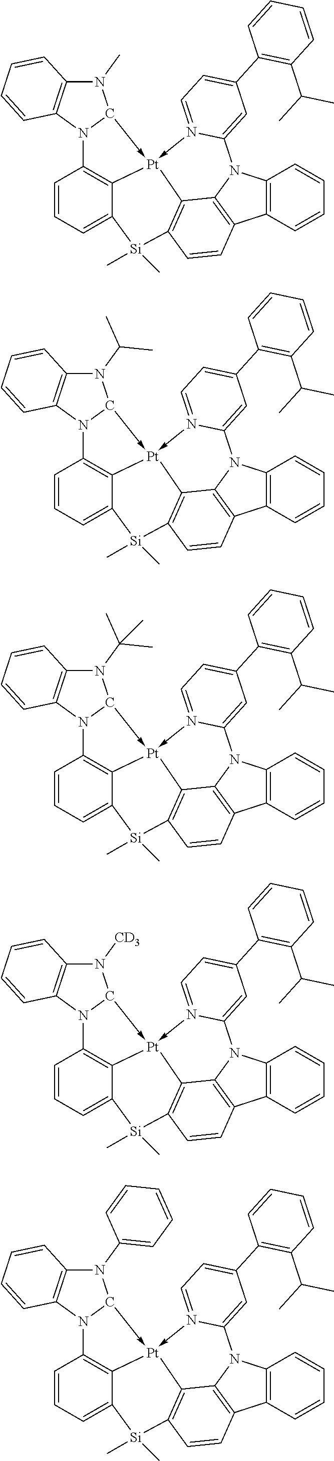

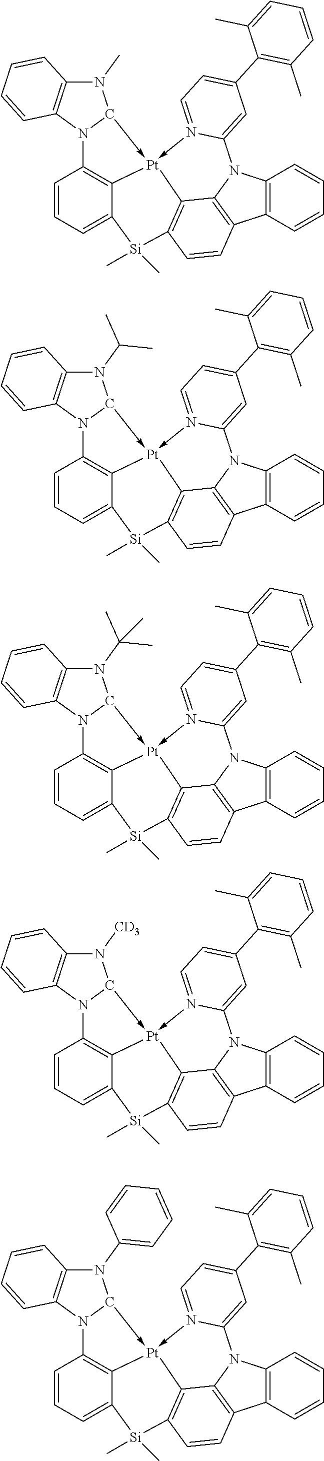

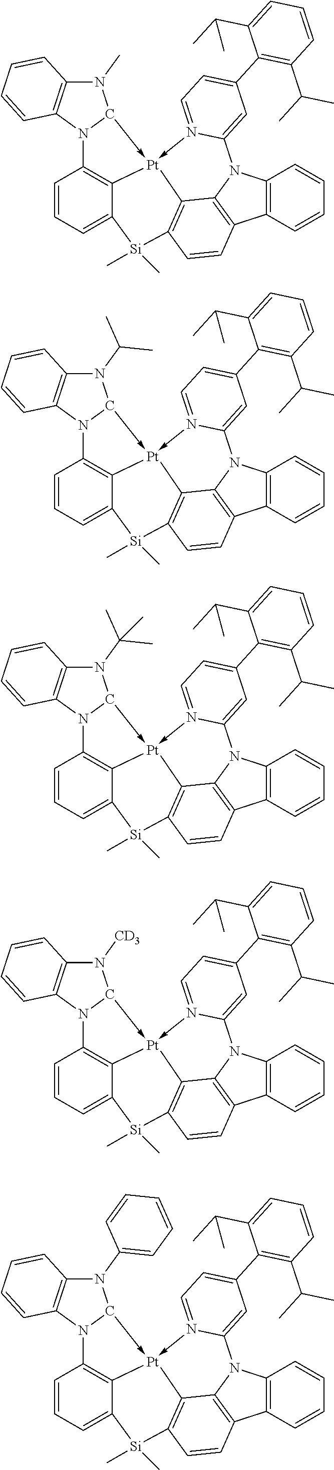

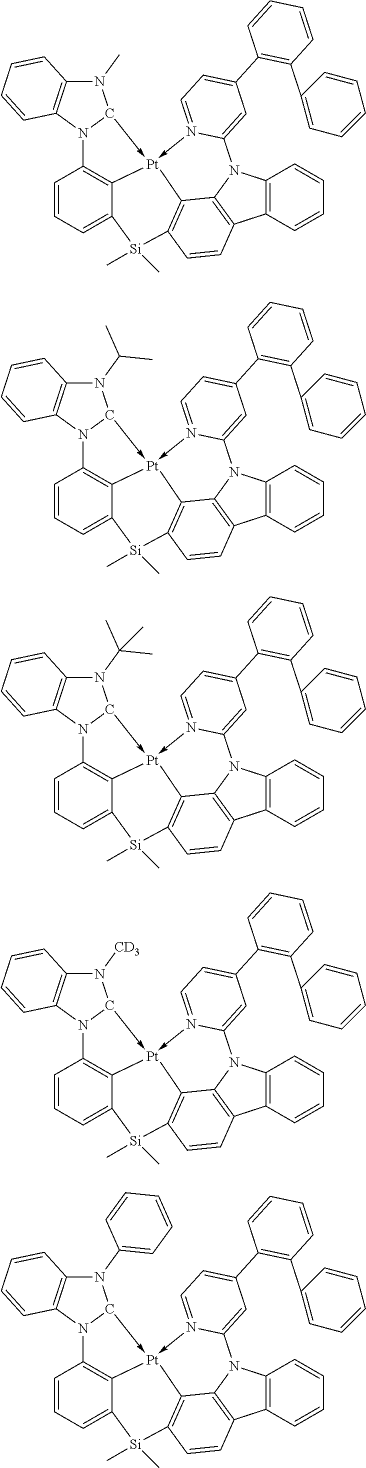

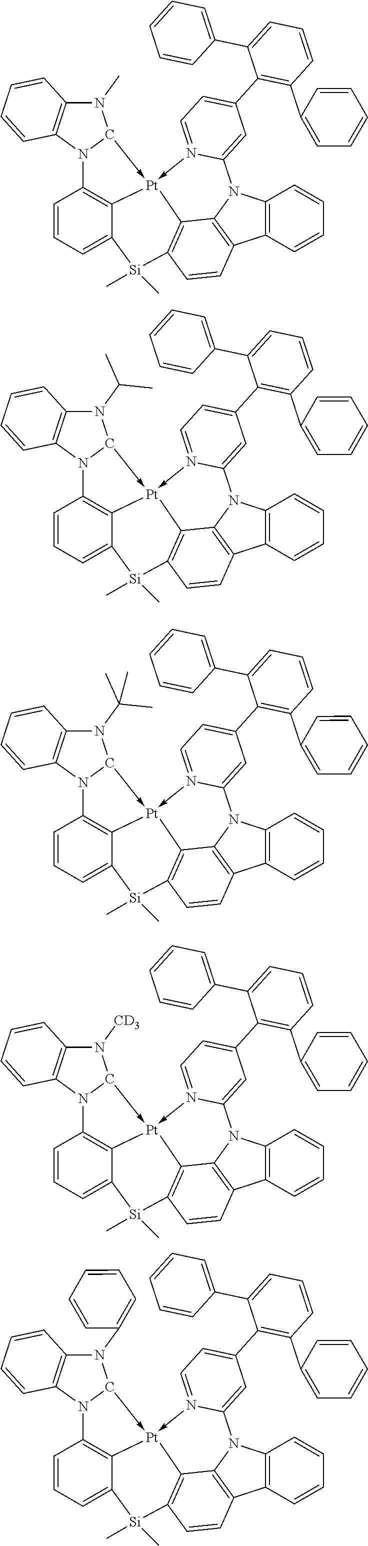

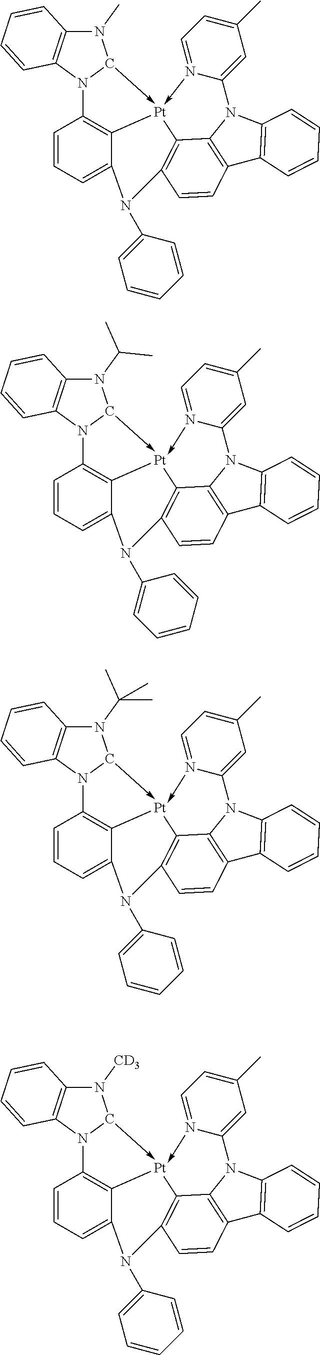

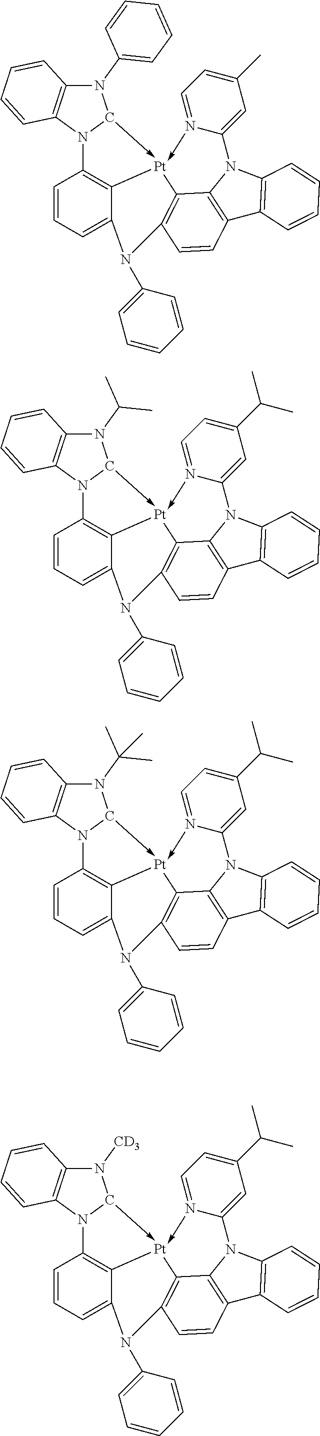

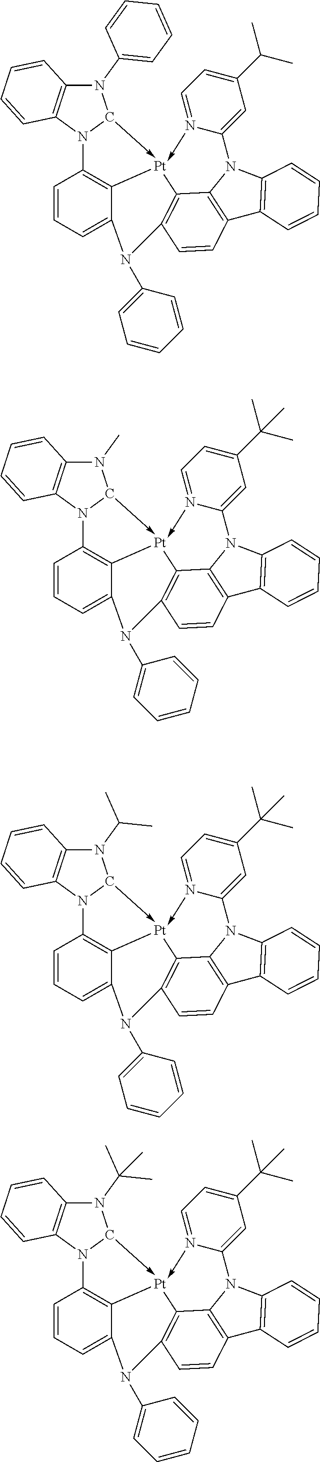

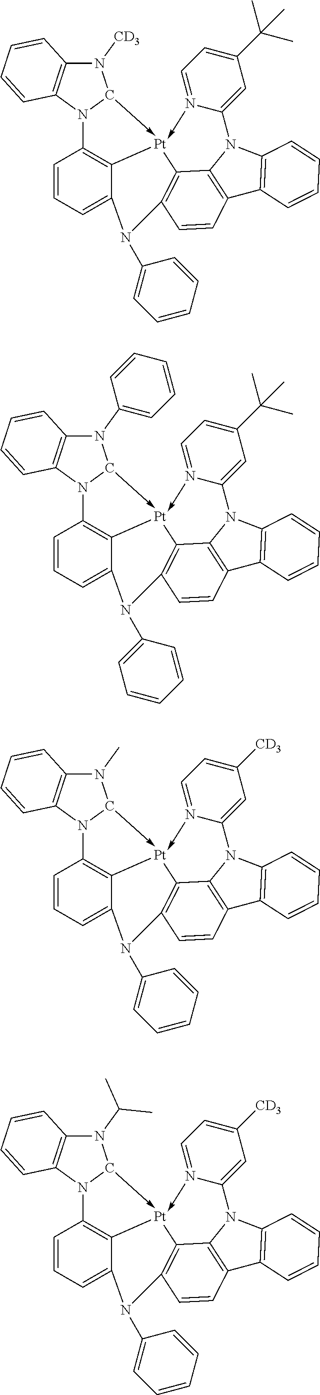

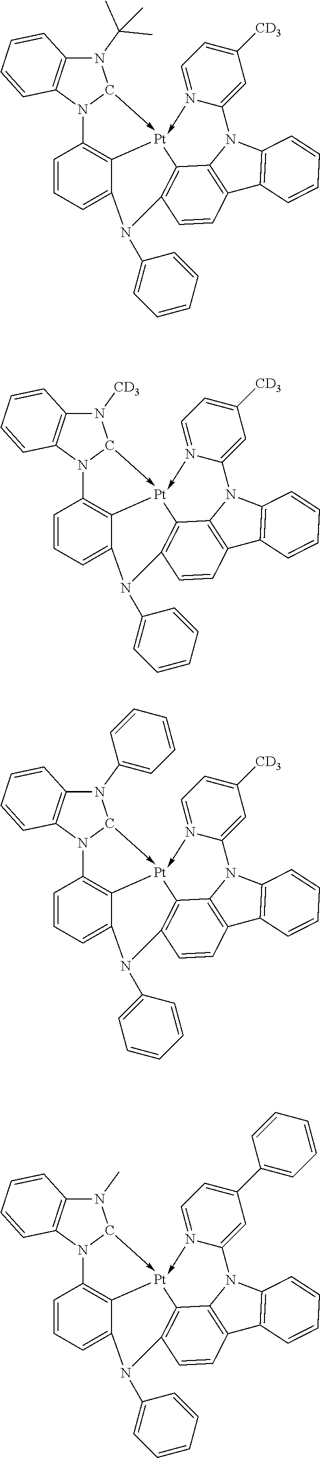

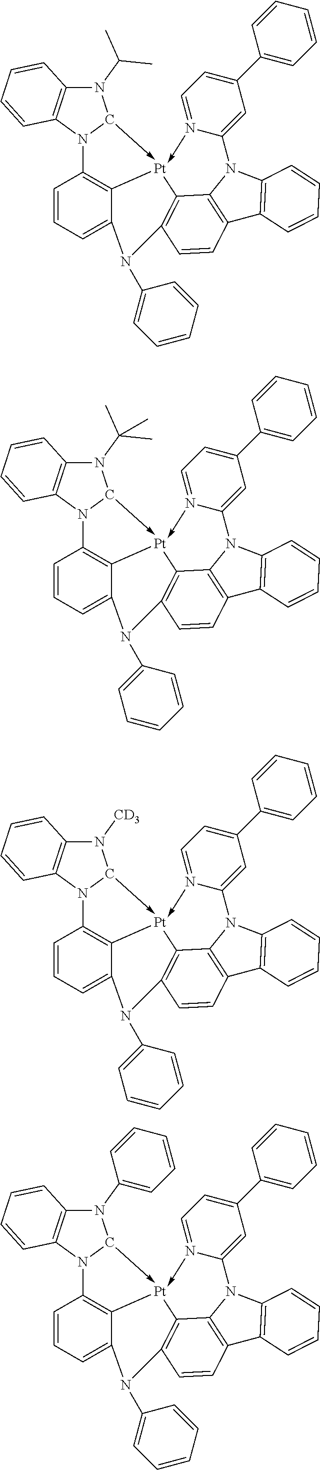

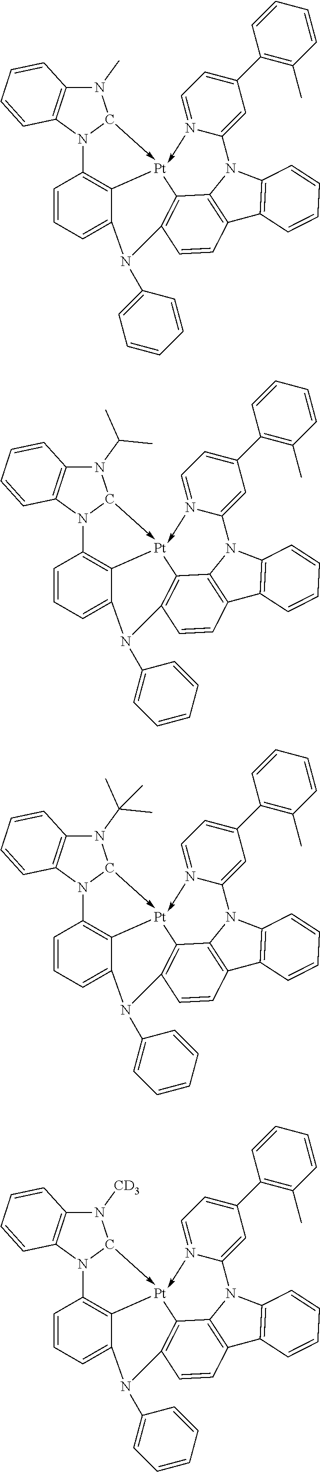

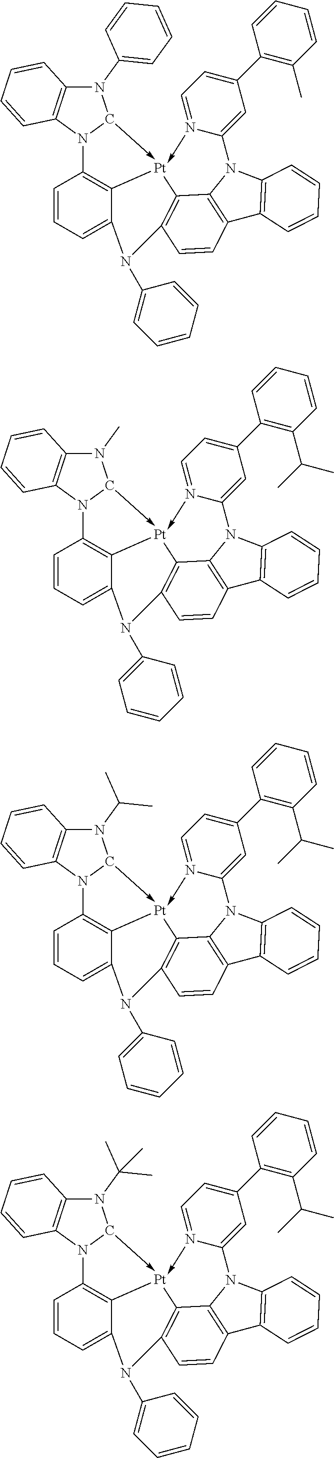

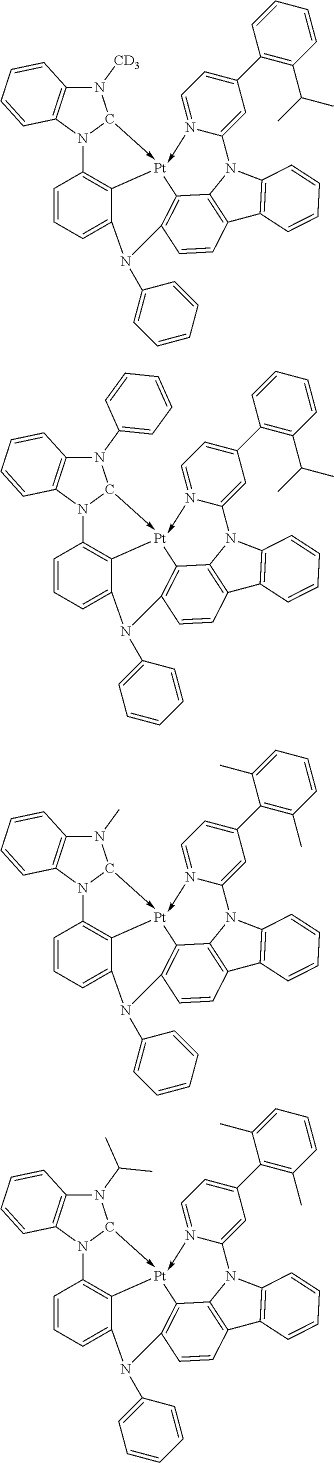

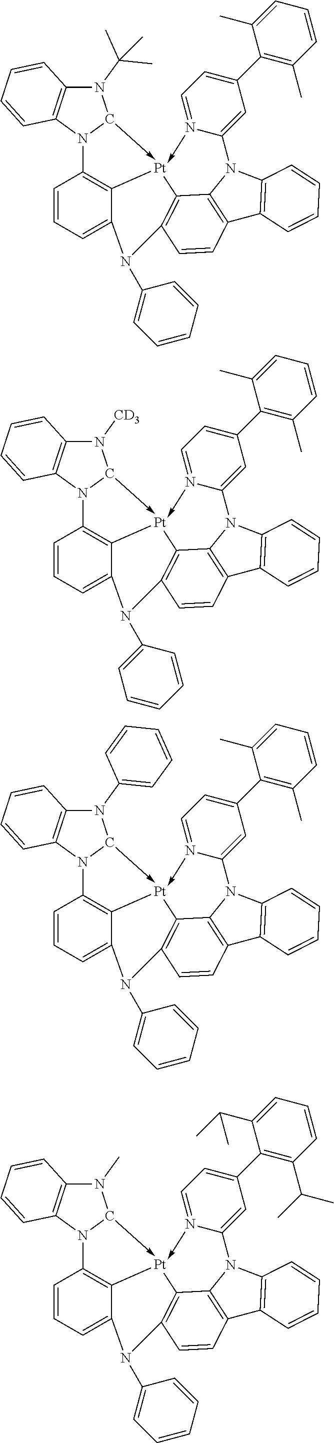

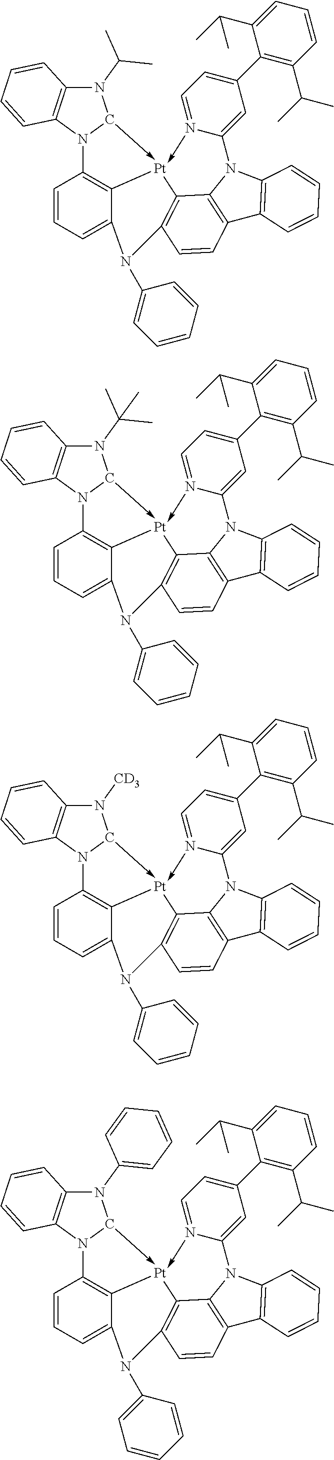

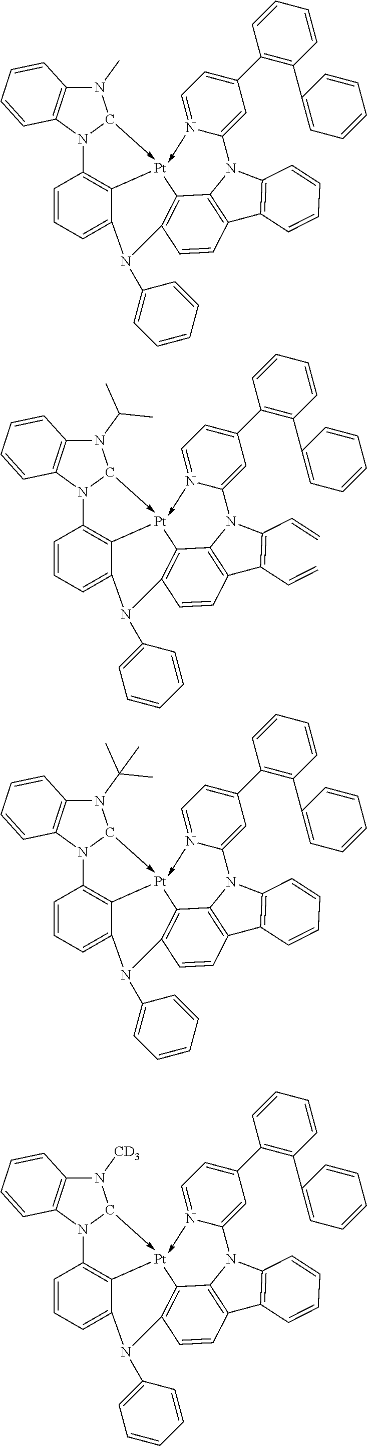

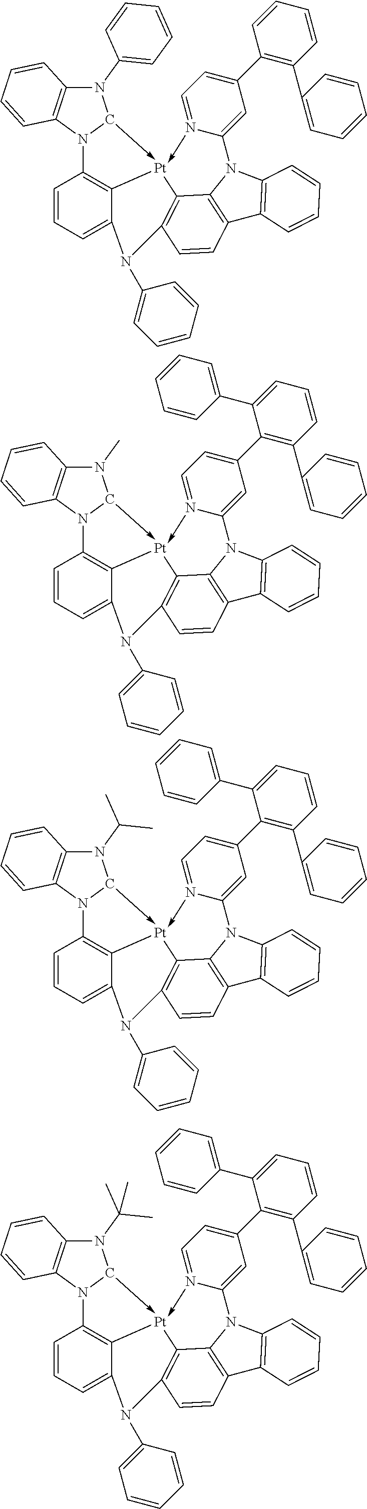

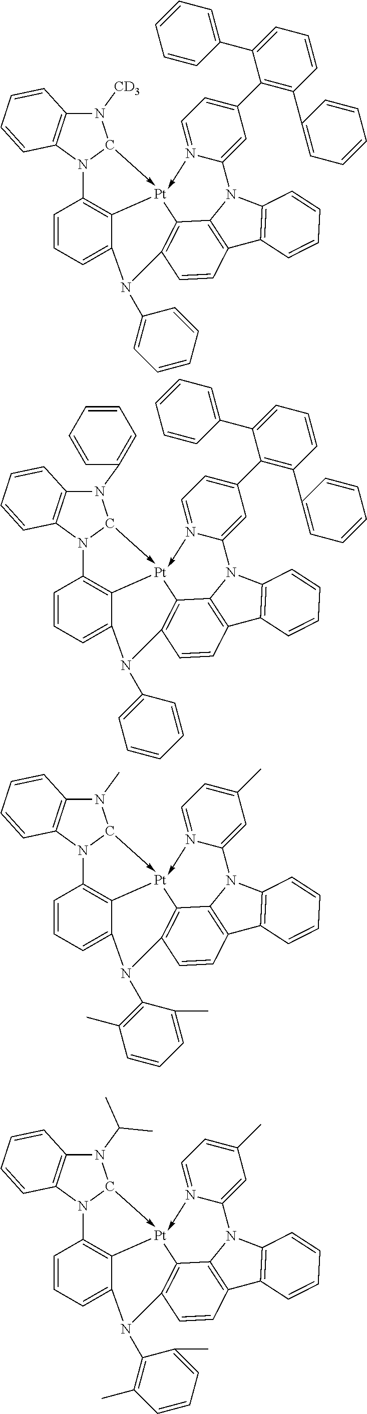

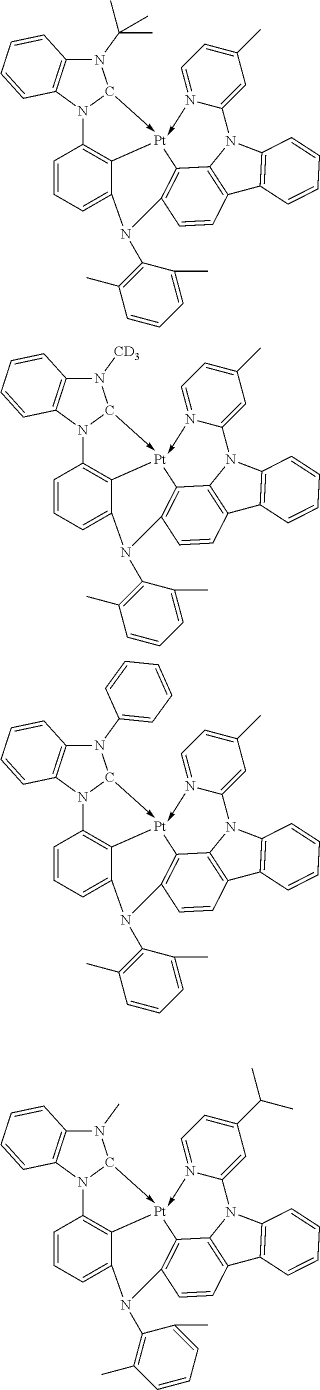

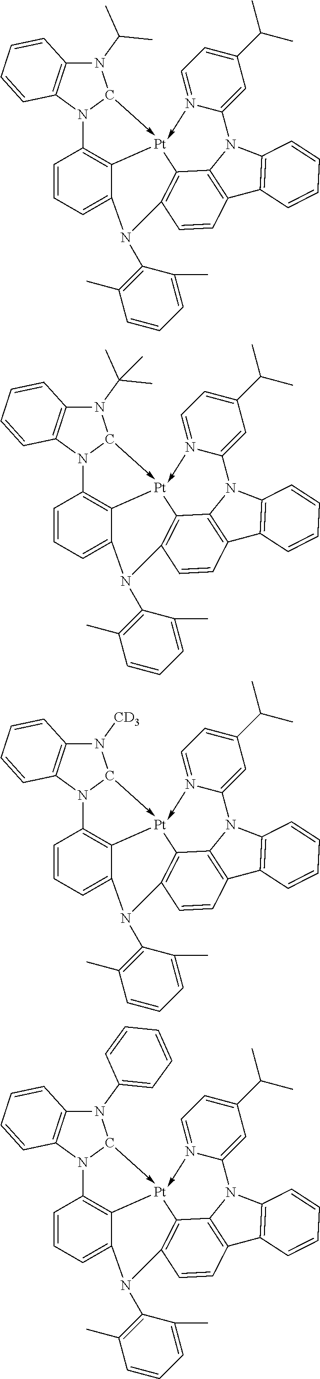

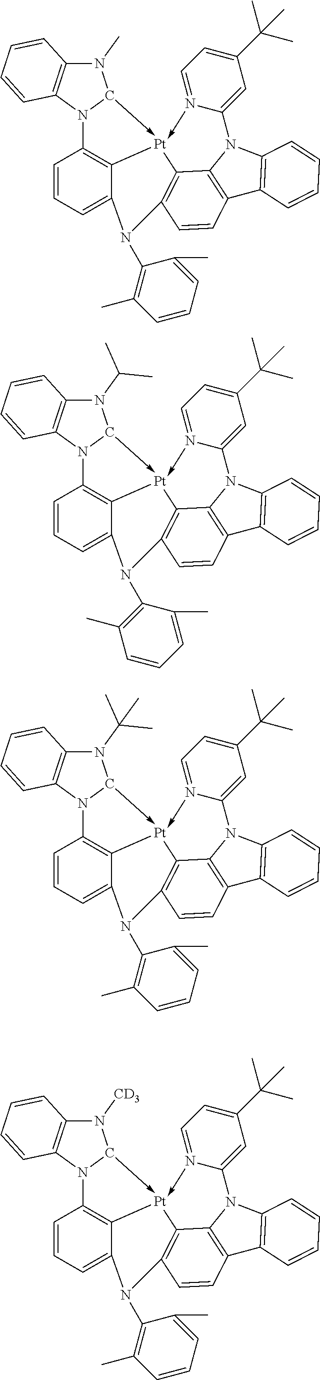

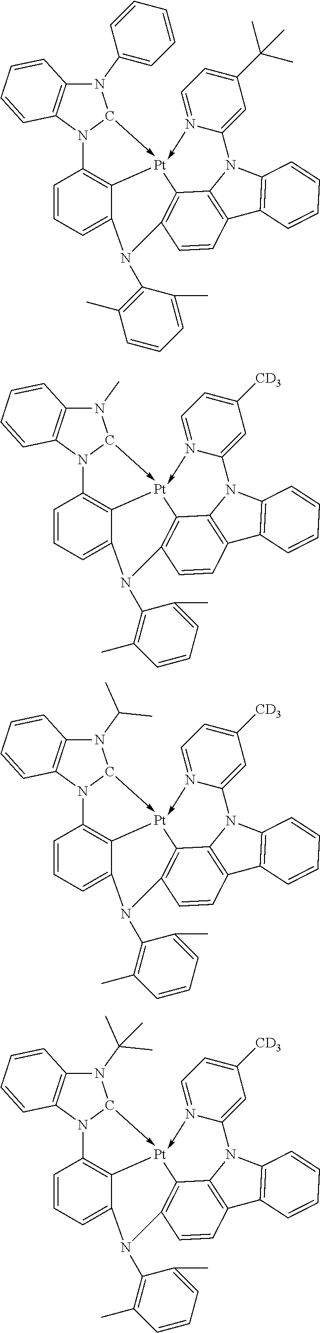

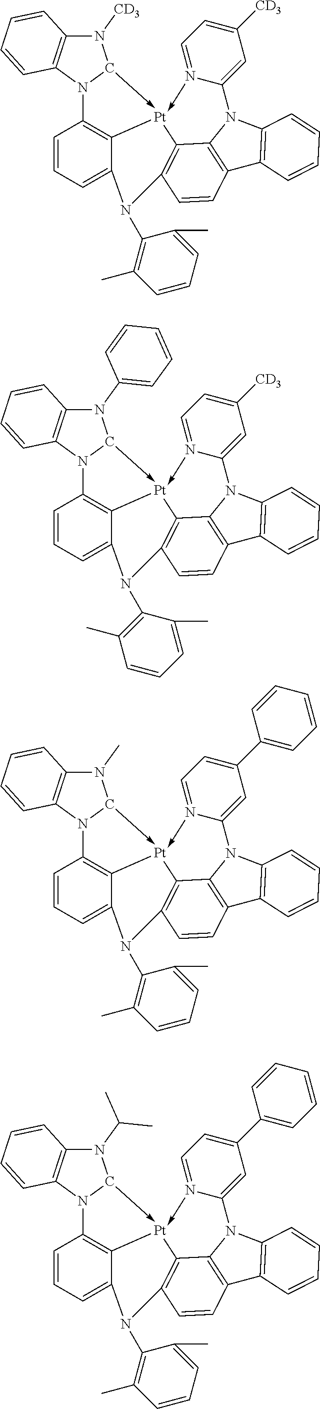

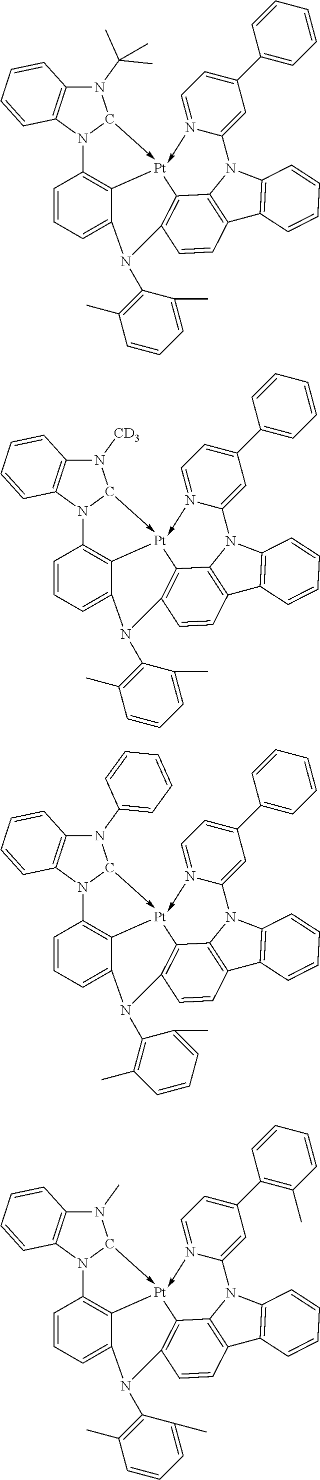

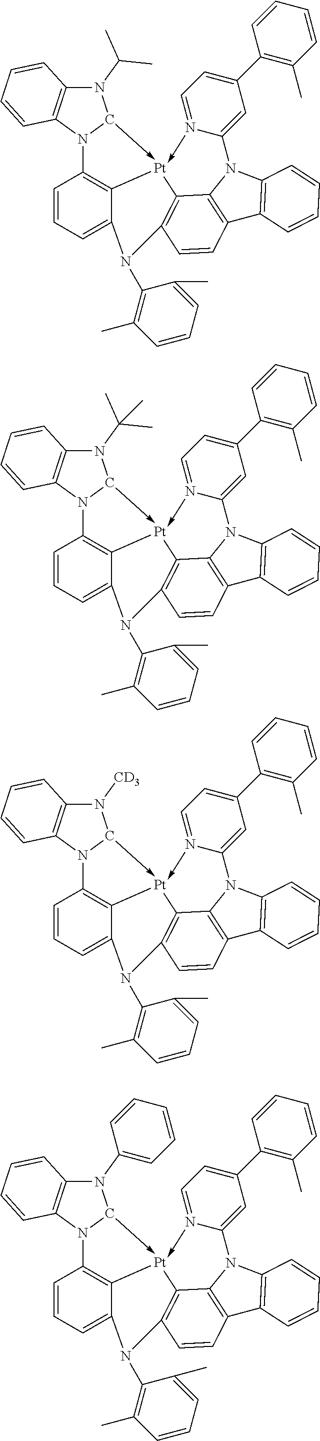

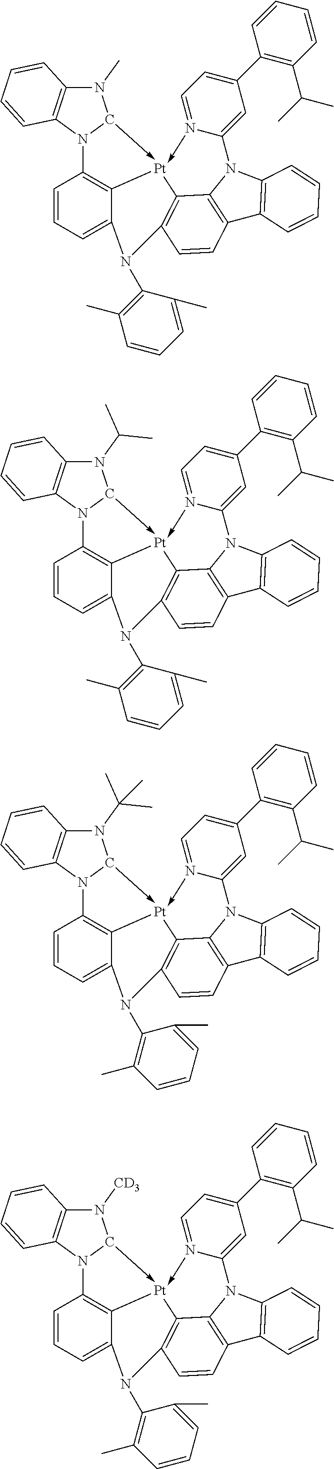

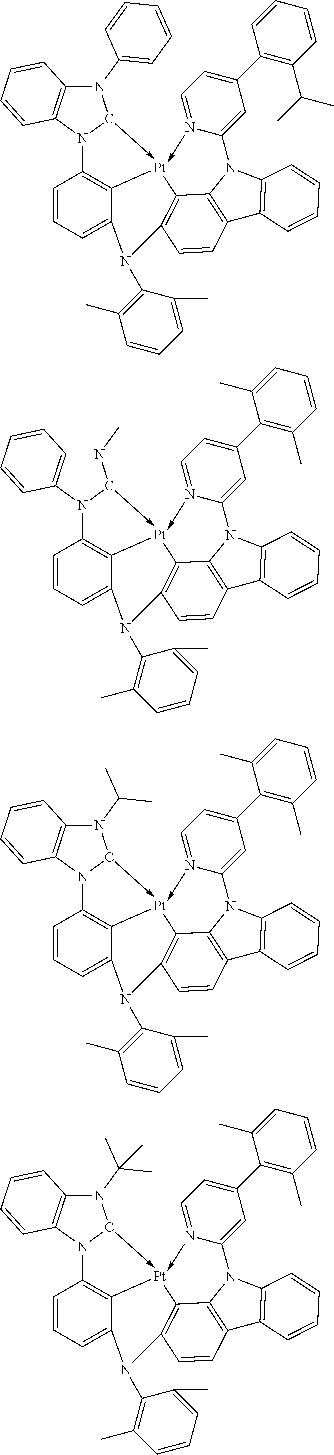

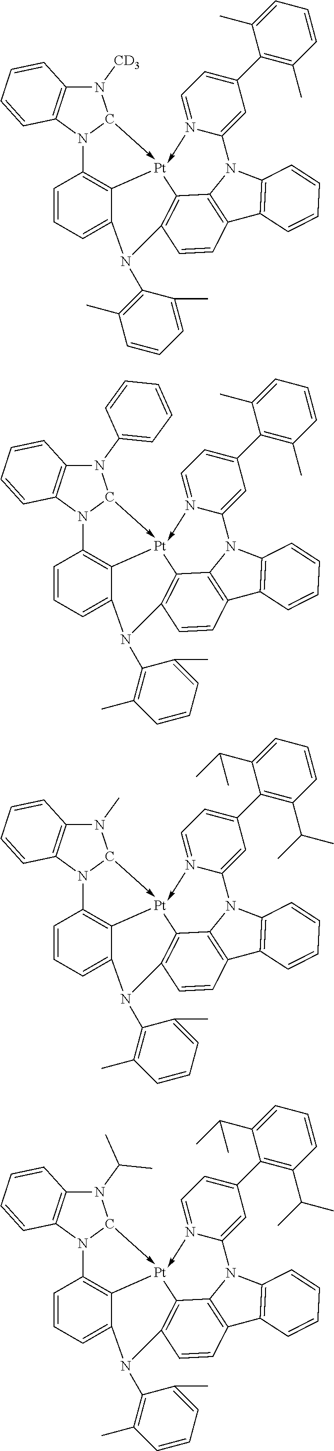

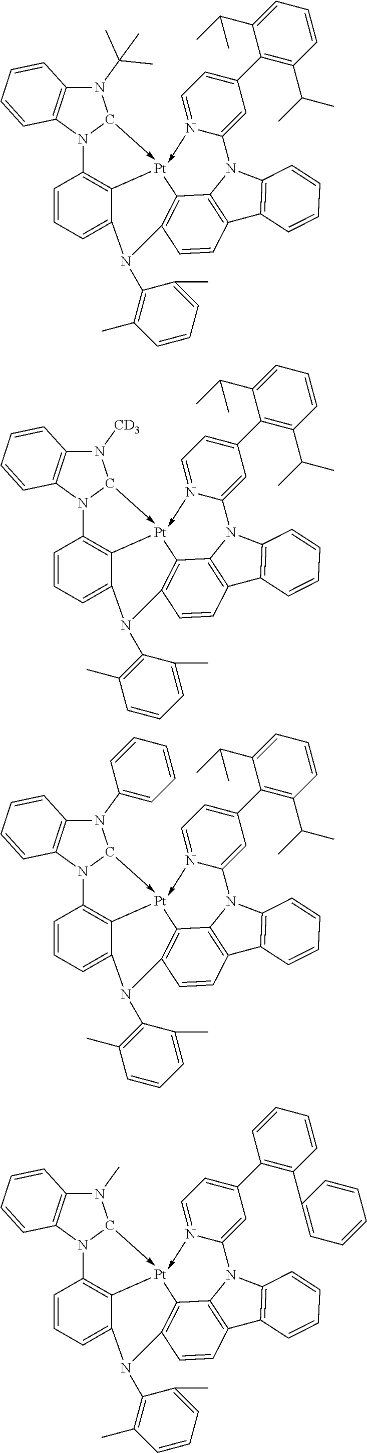

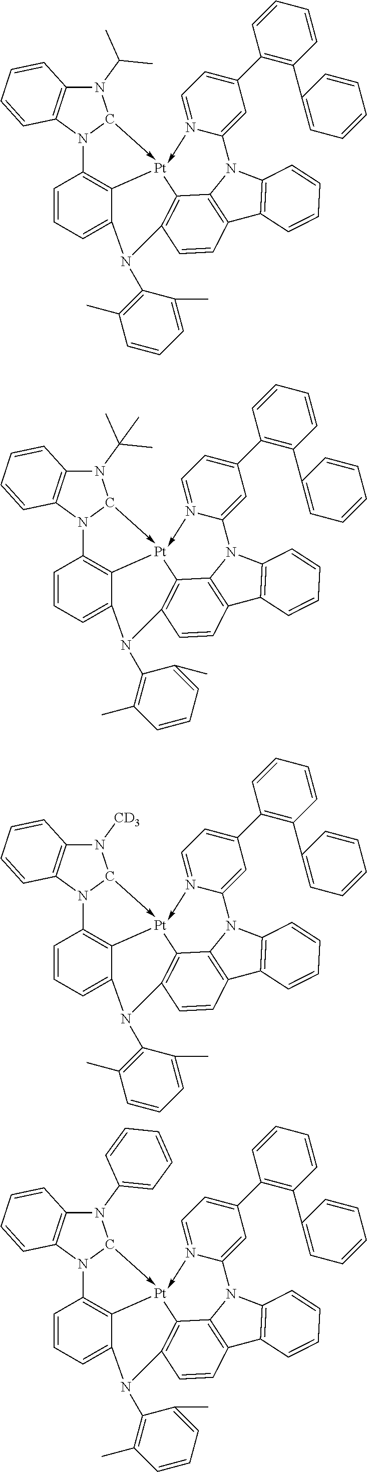

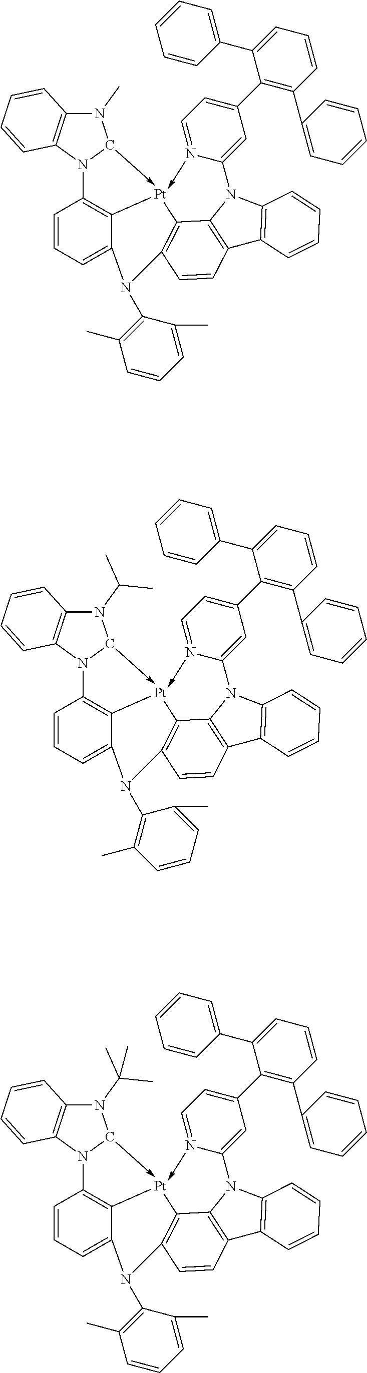

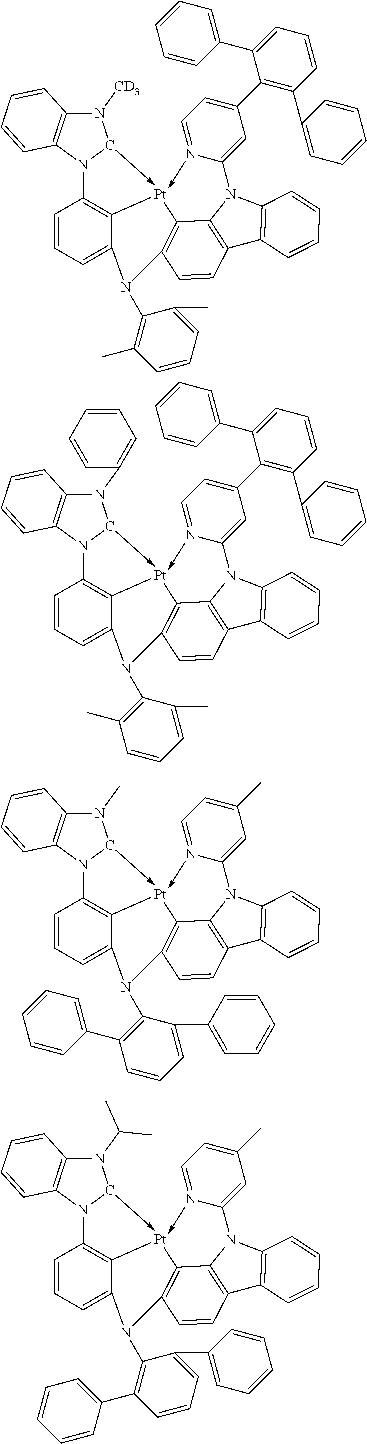

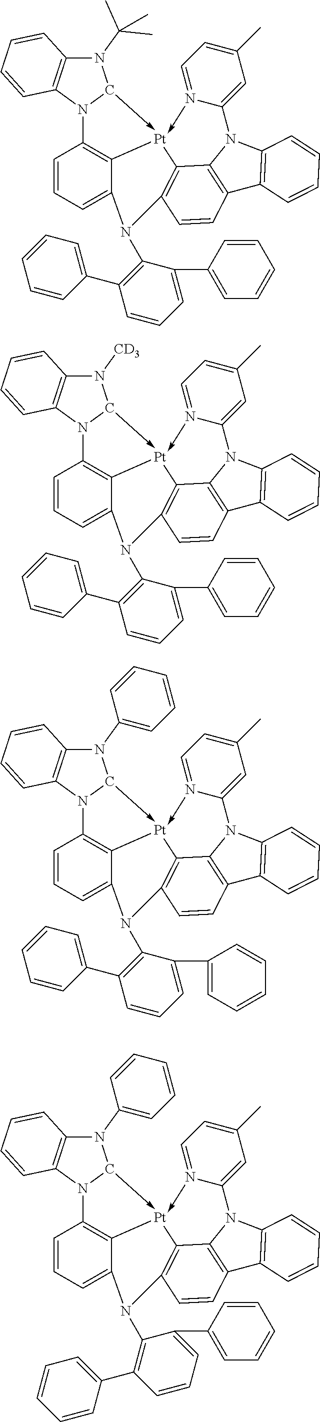

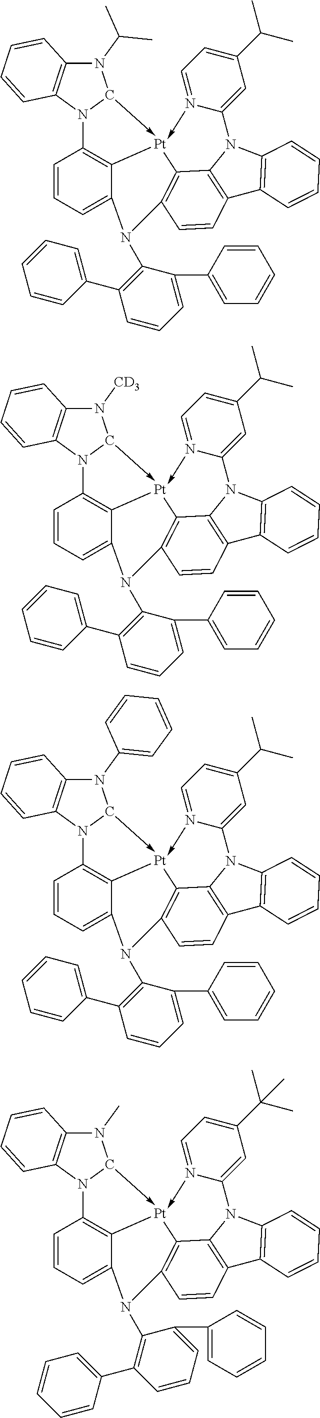

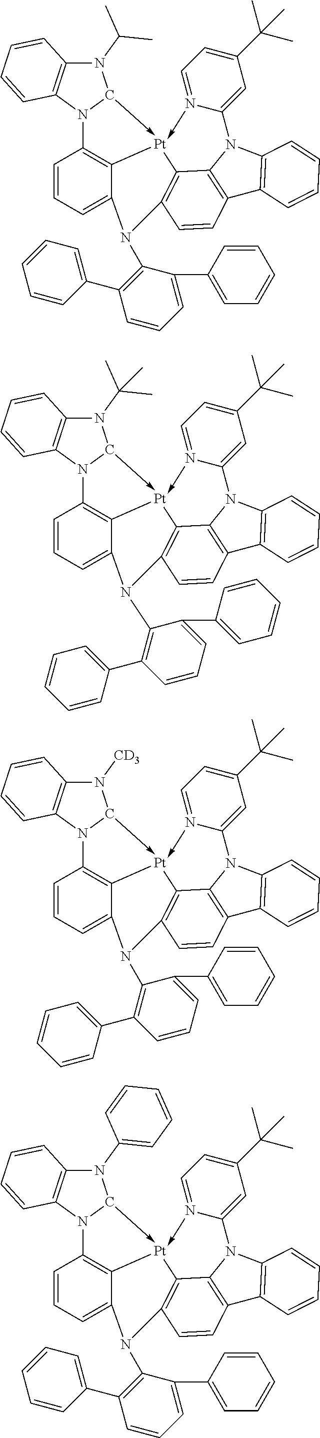

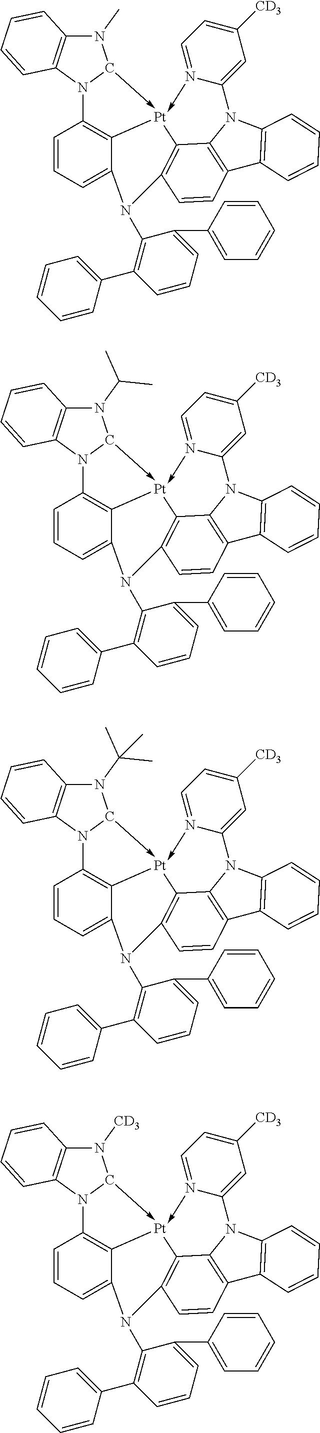

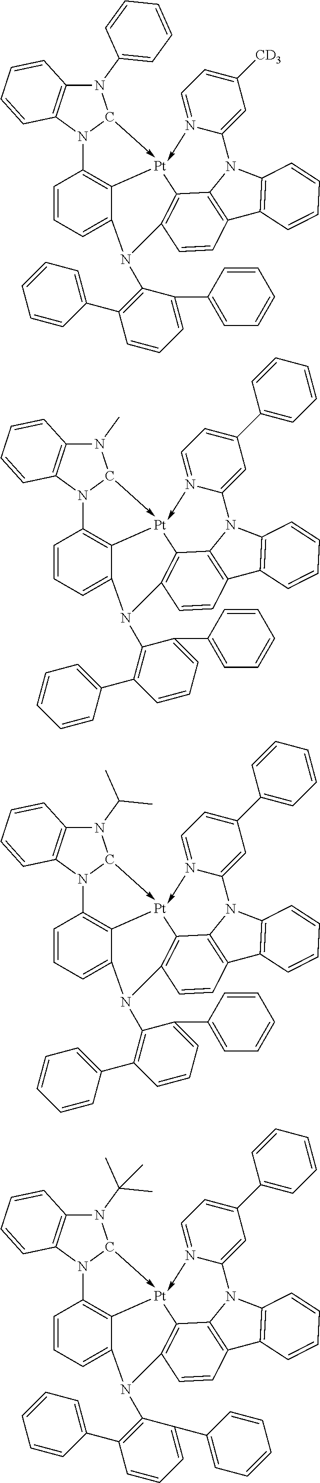

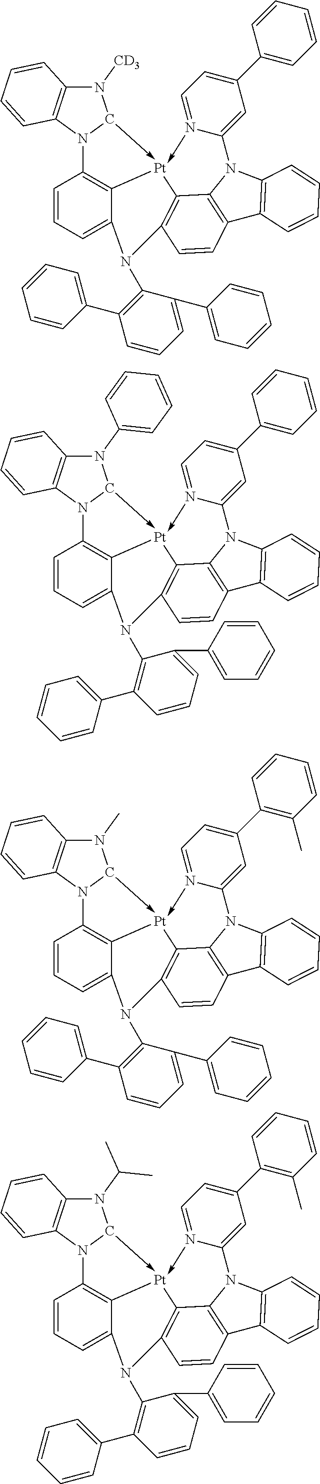

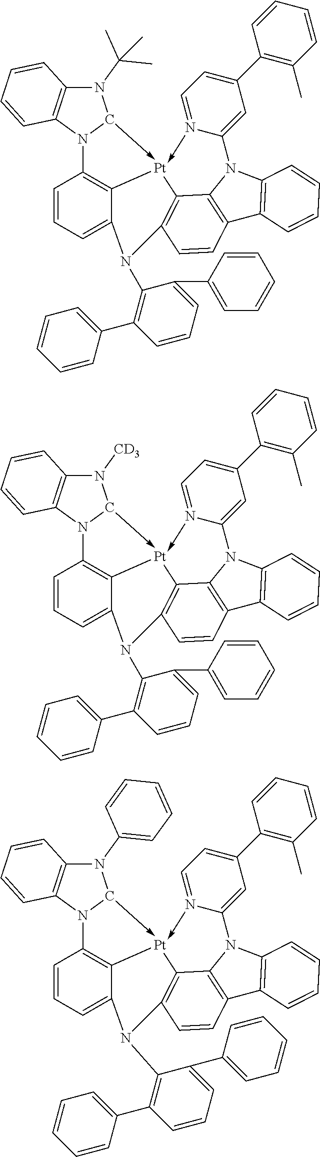

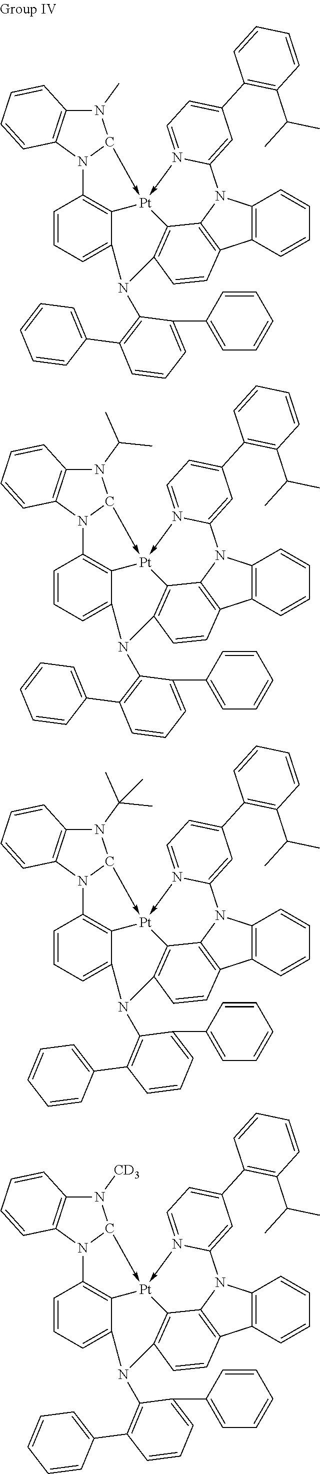

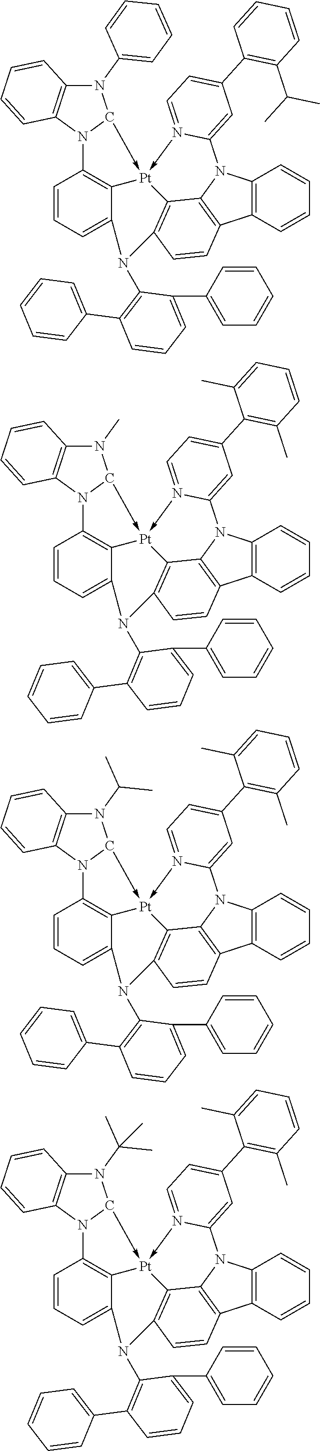

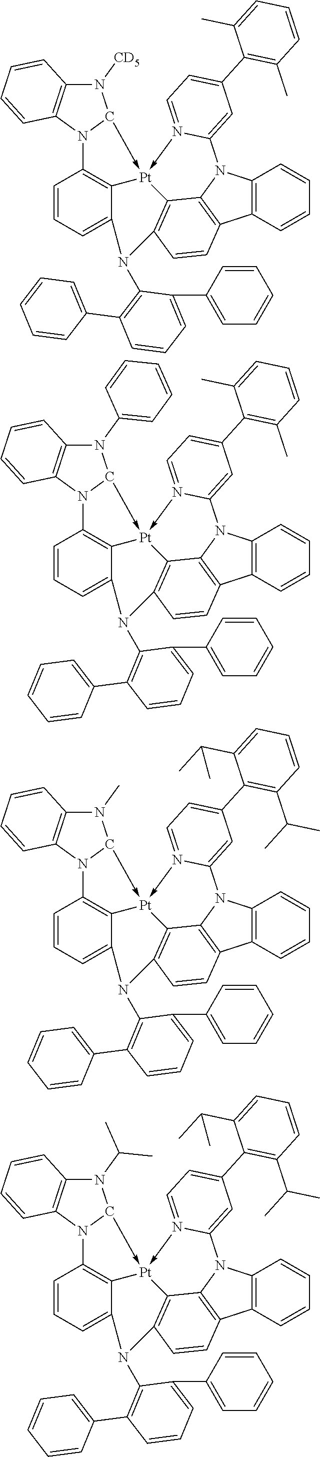

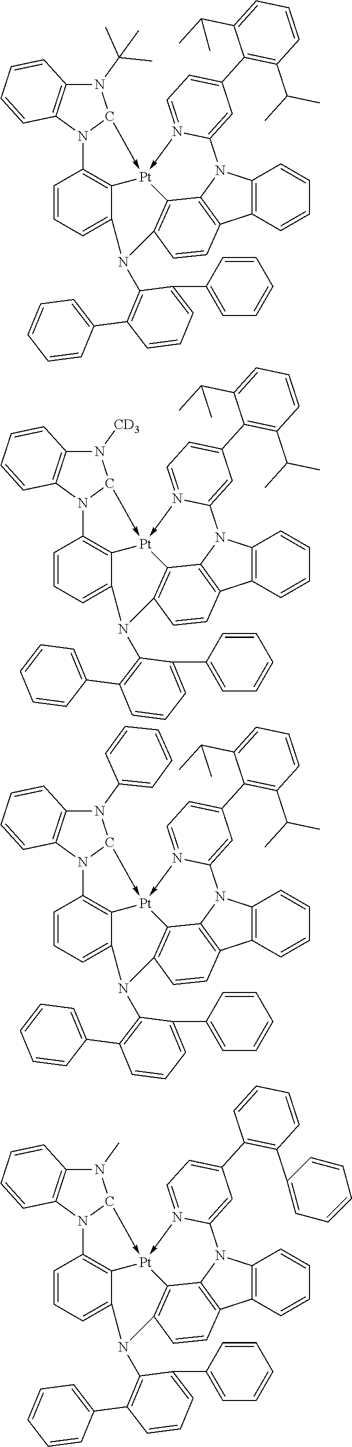

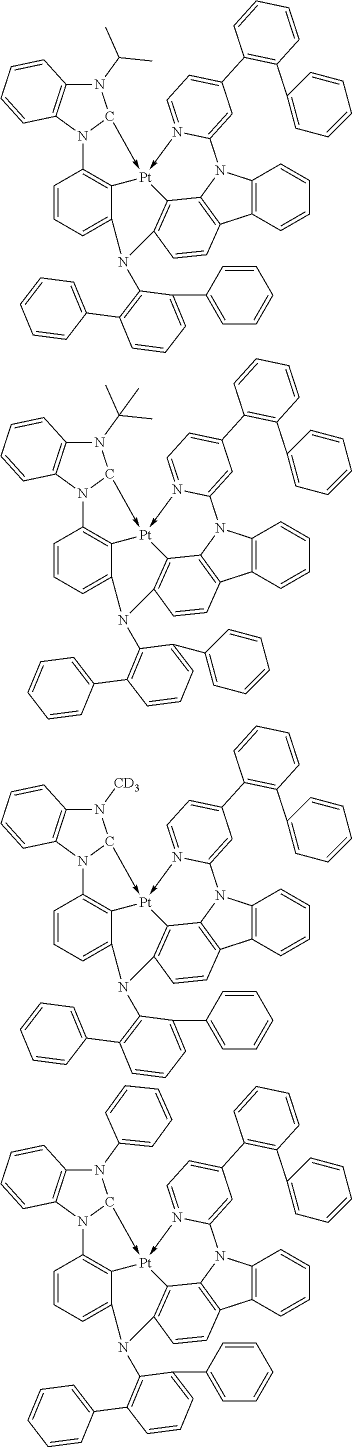

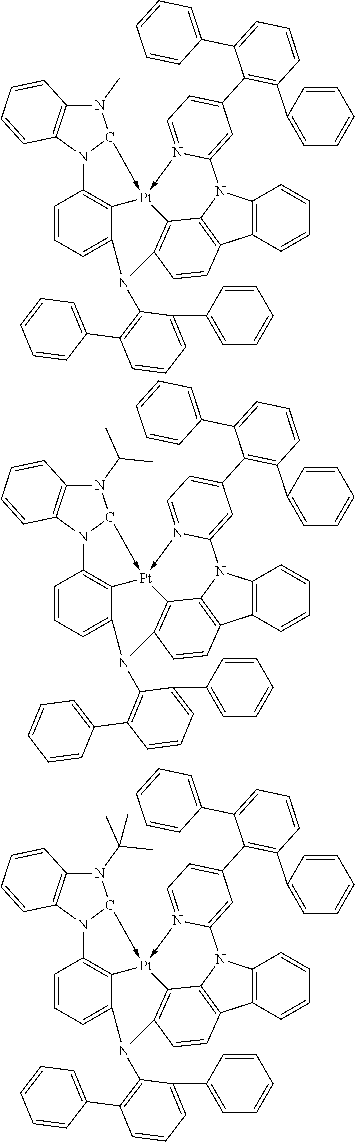

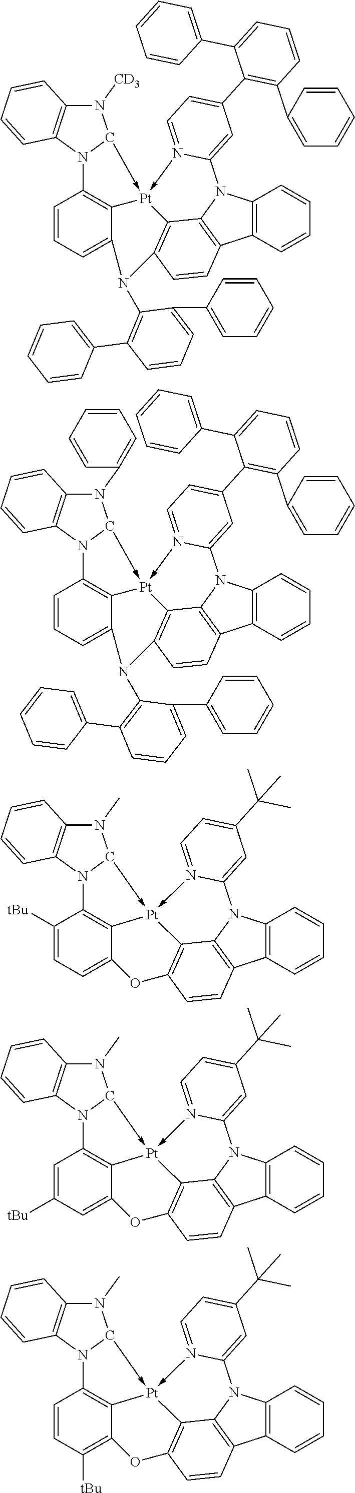

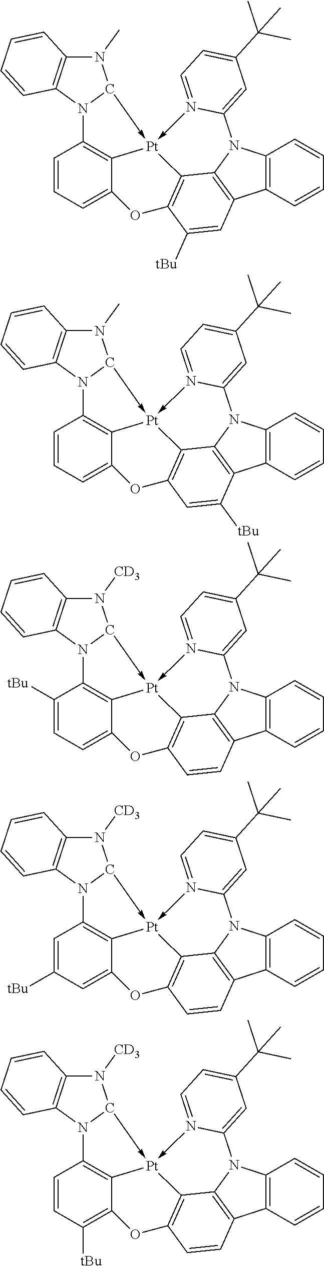

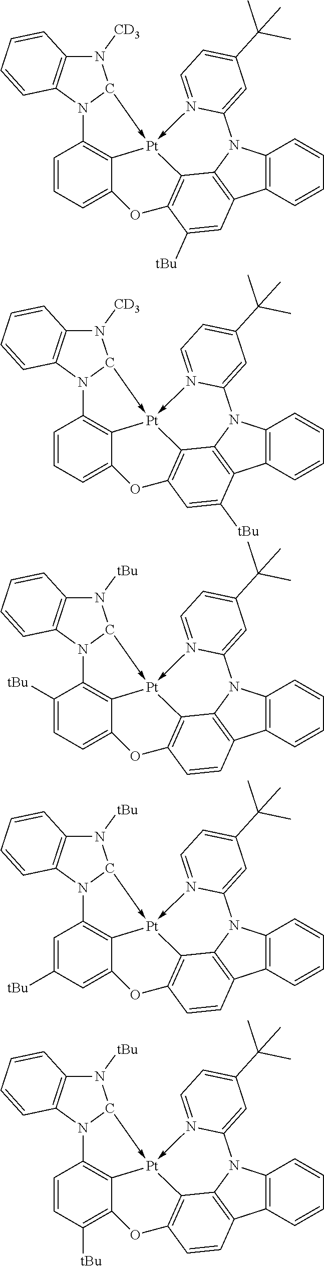

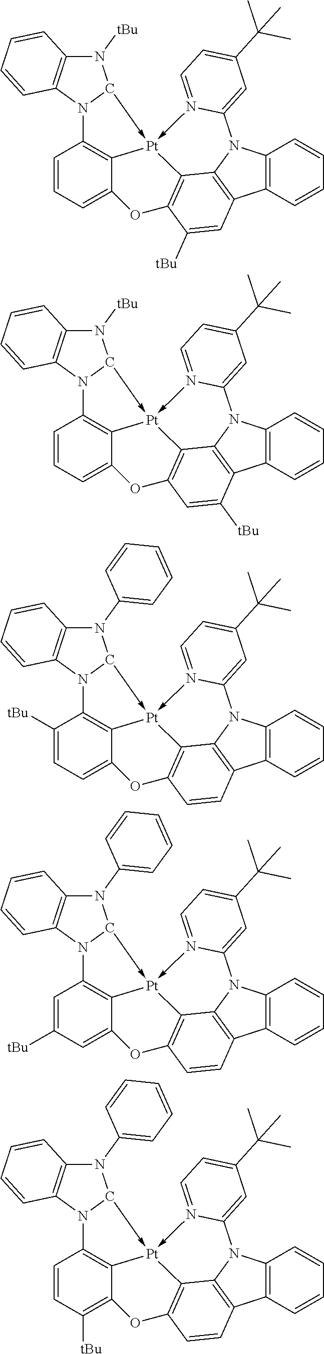

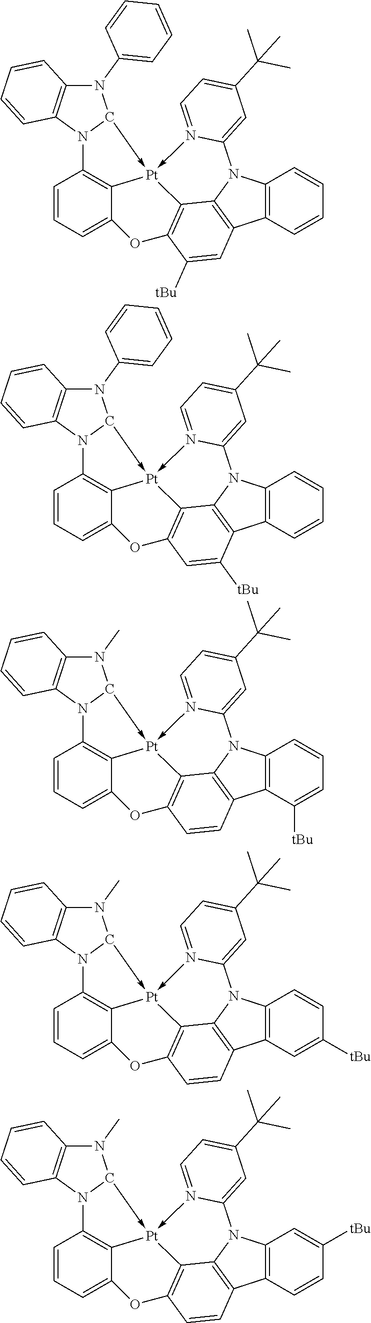

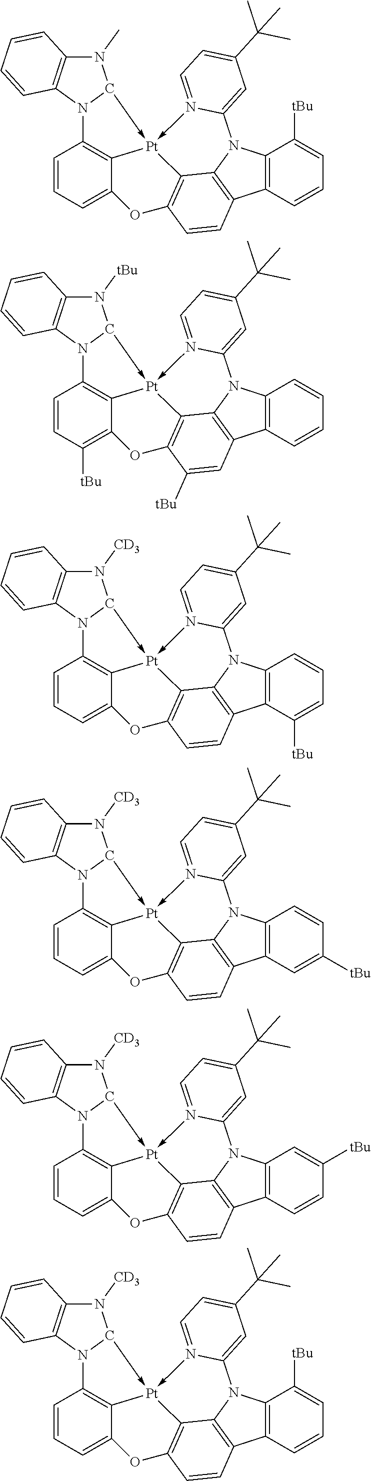

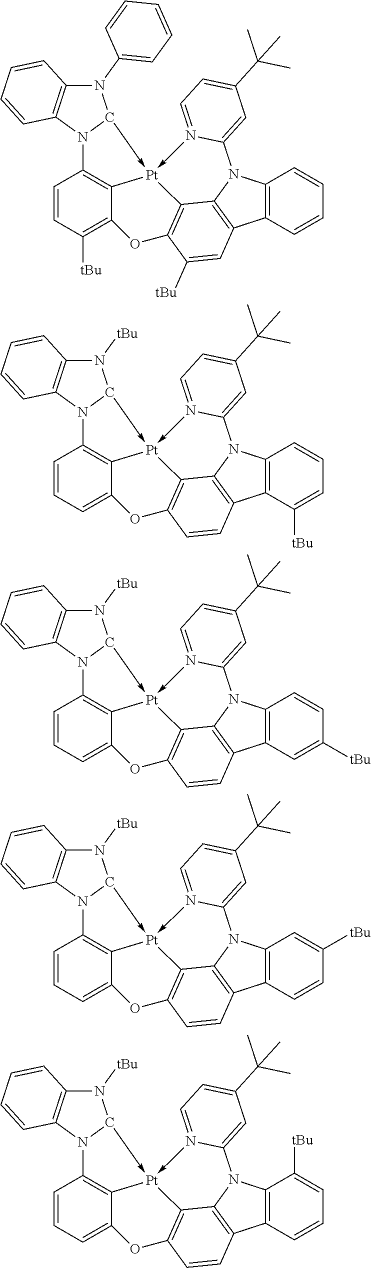

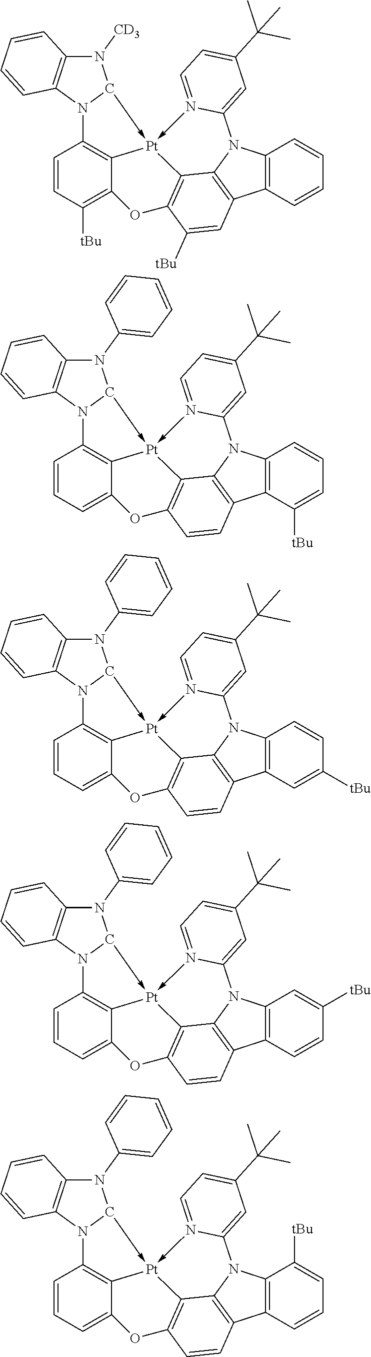

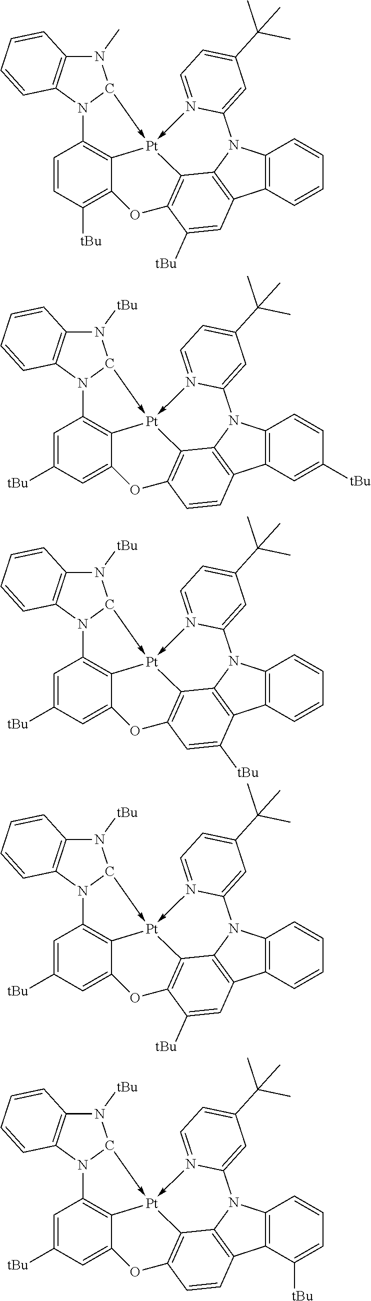

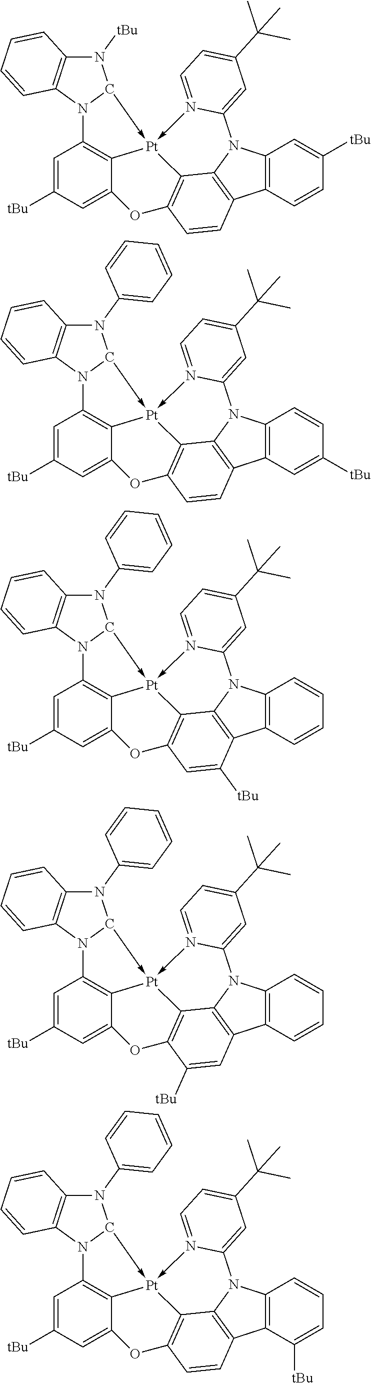

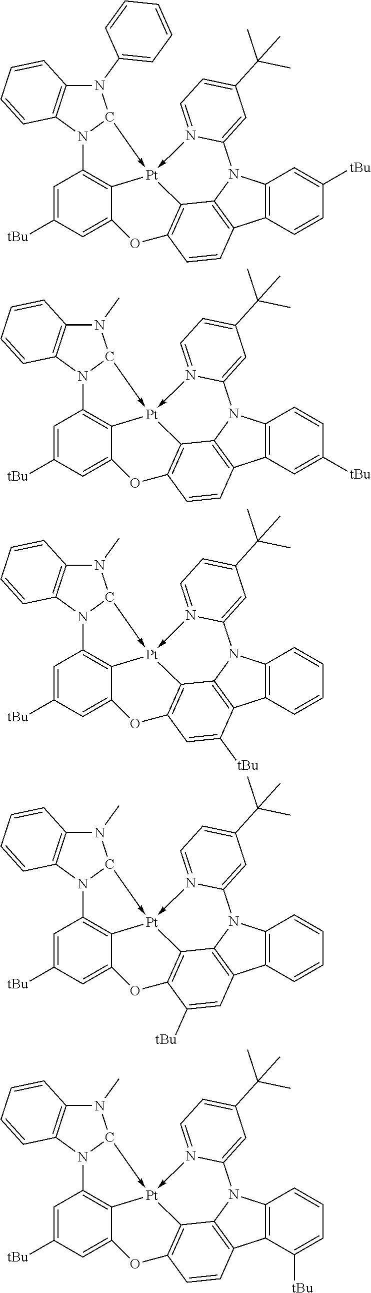

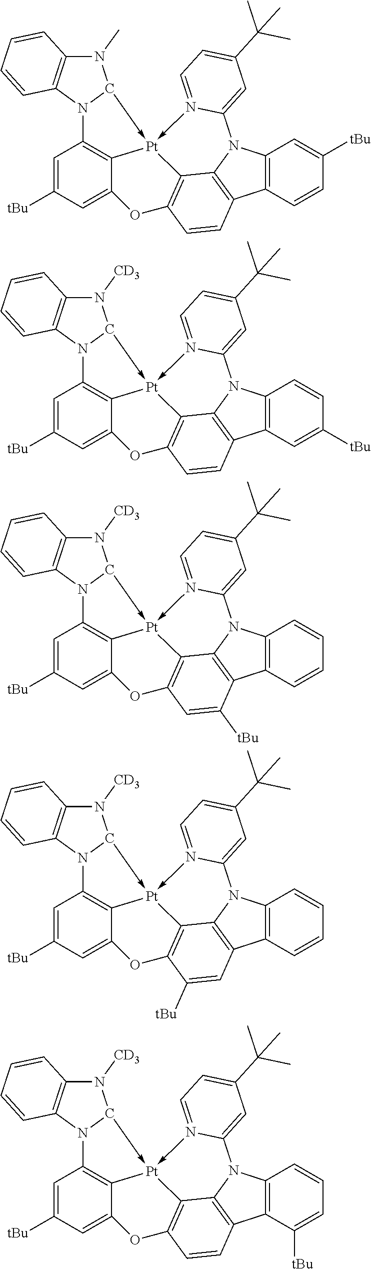

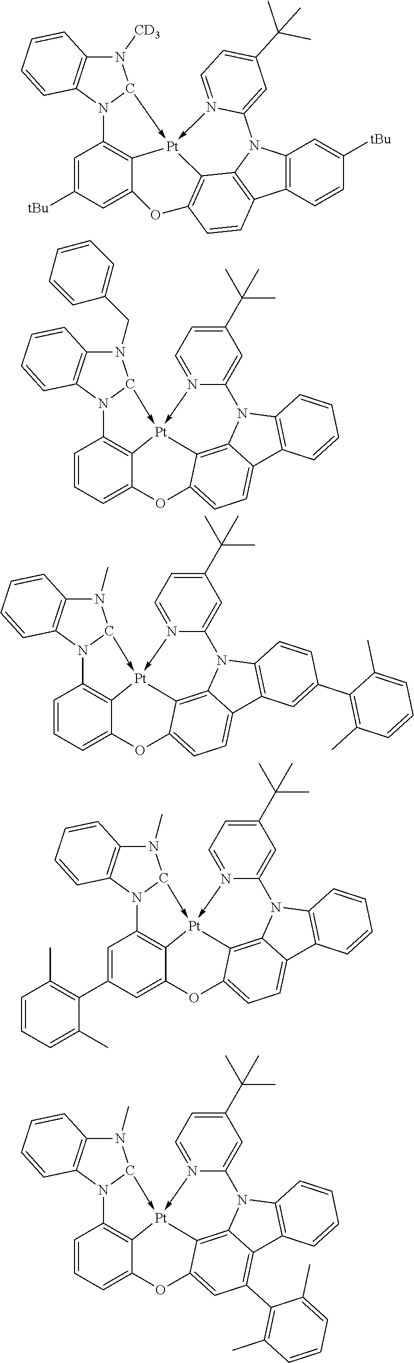

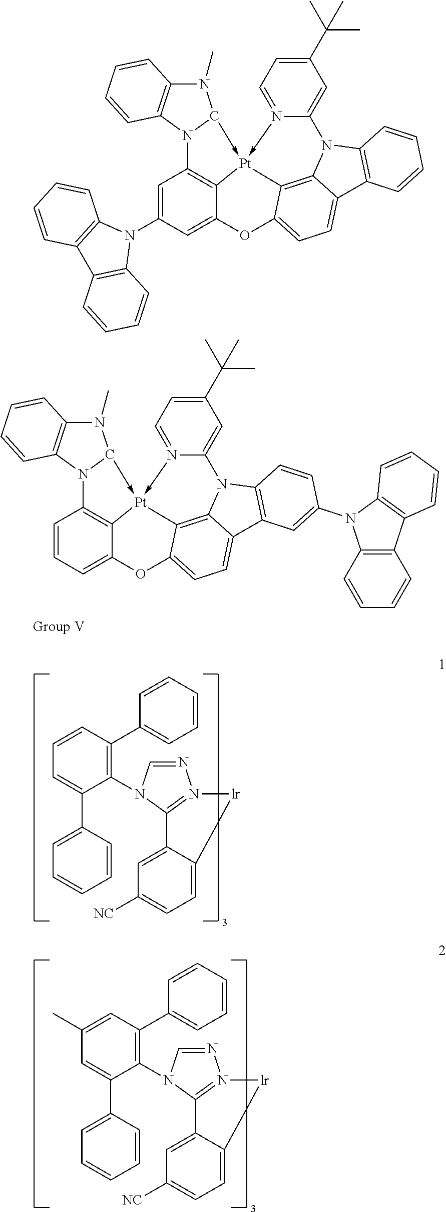

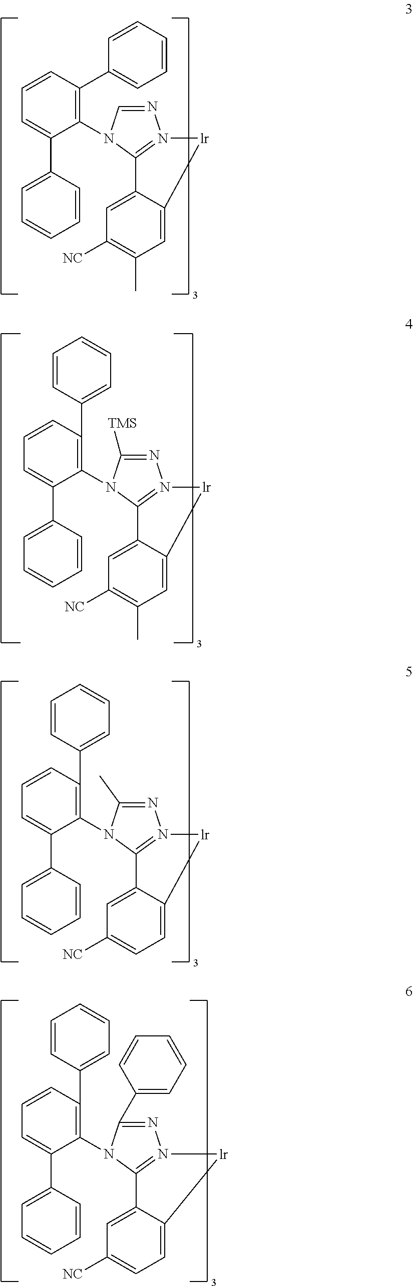

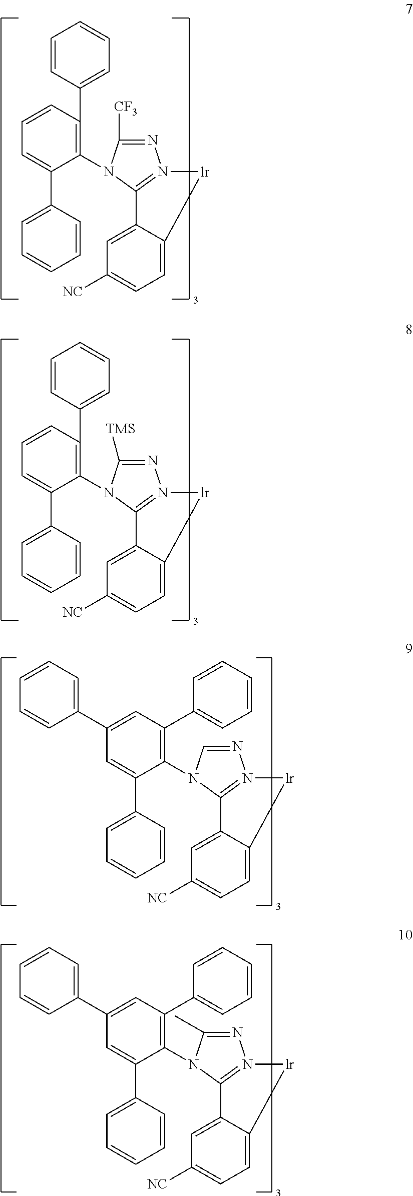

a compound represented by Formula A below:

(L₁₀₁)ₙ₁₀₁-M₁₀₁(L₁₀₂)ₘ₁₀₁ Formula A

wherein,

L₁₀₁, n101, M₁₀₁, L₁₀₂, and m101 in Formula A are the same as described in connection with Tables 1 to 3:

TABLE 1

| Compound Name | L₁₀₁ | n101 | M₁₀₁ | L₁₀₂ | m101 |
|---|---|---|---|---|---|
| BD001 | LM1 | 3 | Ir | — | 0 |
| BD002 | LM2 | 3 | Ir | — | 0 |
| BD003 | LM3 | 3 | Ir | — | 0 |
| BD004 | LM4 | 3 | Ir | — | 0 |
| BD005 | LM5 | 3 | Ir | — | 0 |
| BD006 | LM6 | 3 | Ir | — | 0 |
| BD007 | LM7 | 3 | Ir | — | 0 |
| BD008 | LM8 | 3 | Ir | — | 0 |
| BD009 | LM9 | 3 | Ir | — | 0 |
| BD010 | LM10 | 3 | Ir | — | 0 |
| BD011 | LM11 | 3 | Ir | — | 0 |
| BD012 | LM12 | 3 | Ir | — | 0 |
| BD013 | LM13 | 3 | Ir | — | 0 |
| BD014 | LM14 | 3 | Ir | — | 0 |
| BD015 | LM15 | 3 | Ir | — | 0 |
| BD016 | LM16 | 3 | Ir | — | 0 |
| BD017 | LM17 | 3 | Ir | — | 0 |
| BD018 | LM18 | 3 | Ir | — | 0 |
| BD019 | LM19 | 3 | Ir | — | 0 |
| BD020 | LM20 | 3 | Ir | — | 0 |
| BD021 | LM21 | 3 | Ir | — | 0 |
| BD022 | LM22 | 3 | Ir | — | 0 |
| BD023 | LM23 | 3 | Ir | — | 0 |
| BD024 | LM24 | 3 | Ir | — | 0 |
| BD025 | LM25 | 3 | Ir | — | 0 |
| BD026 | LM26 | 3 | Ir | — | 0 |
| BD027 | LM27 | 3 | Ir | — | 0 |
| BD028 | LM28 | 3 | Ir | — | 0 |
| BD029 | LM29 | 3 | Ir | — | 0 |
| BD030 | LM30 | 3 | Ir | — | 0 |
| BD031 | LM31 | 3 | Ir | — | 0 |
| BD032 | LM32 | 3 | Ir | — | 0 |
| BD033 | LM33 | 3 | Ir | — | 0 |
| BD034 | LM34 | 3 | Ir | — | 0 |
| BD035 | LM35 | 3 | Ir | — | 0 |
| BD036 | LM36 | 3 | Ir | — | 0 |
| BD037 | LM37 | 3 | Ir | — | 0 |
| BD038 | LM38 | 3 | Ir | — | 0 |
| BD039 | LM39 | 3 | Ir | — | 0 |
| BD040 | LM40 | 3 | Ir | — | 0 |
| BD041 | LM41 | 3 | Ir | — | 0 |
| BD042 | LM42 | 3 | Ir | — | 0 |
| BD043 | LM43 | 3 | Ir | — | 0 |
| BD044 | LM44 | 3 | Ir | — | 0 |
| BD045 | LM45 | 3 | Ir | — | 0 |
| BD046 | LM46 | 3 | Ir | — | 0 |
| BD047 | LM47 | 3 | Ir | — | 0 |
| BD048 | LM48 | 3 | Ir | — | 0 |
| BD049 | LM49 | 3 | Ir | — | 0 |
| BD050 | LM50 | 3 | Ir | — | 0 |
| BD051 | LM51 | 3 | Ir | — | 0 |
| BD052 | LM52 | 3 | Ir | — | 0 |
| BD053 | LM53 | 3 | Ir | — | 0 |
| BD054 | LM54 | 3 | Ir | — | 0 |
| BD055 | LM55 | 3 | Ir | — | 0 |
| BD056 | LM56 | 3 | Ir | — | 0 |
| BD057 | LM57 | 3 | Ir | — | 0 |
| BD058 | LM58 | 3 | Ir | — | 0 |
| BD059 | LM59 | 3 | Ir | — | 0 |
| BD060 | LM60 | 3 | Ir | — | 0 |
| BD061 | LM61 | 3 | Ir | — | 0 |
| BD062 | LM62 | 3 | Ir | — | 0 |
| BD063 | LM63 | 3 | Ir | — | 0 |
| BD064 | LM64 | 3 | Ir | — | 0 |
| BD065 | LM65 | 3 | Ir | — | 0 |
| BD066 | LM66 | 3 | Ir | — | 0 |
| BD067 | LM67 | 3 | Ir | — | 0 |
| BD068 | LM68 | 3 | Ir | — | 0 |
| BD069 | LM69 | 3 | Ir | — | 0 |
| BD070 | LM70 | 3 | Ir | — | 0 |
| BD071 | LM71 | 3 | Ir | — | 0 |
| BD072 | LM72 | 3 | Ir | — | 0 |
| BD073 | LM73 | 3 | Ir | — | 0 |
| BD074 | LM74 | 3 | Ir | — | 0 |
| BD075 | LM75 | 3 | Ir | — | 0 |
| BD076 | LM76 | 3 | Ir | — | 0 |
| BD077 | LM77 | 3 | Ir | — | 0 |
| BD078 | LM78 | 3 | Ir | — | 0 |
| BD079 | LM79 | 3 | Ir | — | 0 |
| BD080 | LM80 | 3 | Ir | — | 0 |
| BD081 | LM81 | 3 | Ir | — | 0 |
| BD082 | LM82 | 3 | Ir | — | 0 |
| BD083 | LM83 | 3 | Ir | — | 0 |
| BD084 | LM84 | 3 | Ir | — | 0 |
| BD085 | LM85 | 3 | Ir | — | 0 |
| BD086 | LM86 | 3 | Ir | — | 0 |
| BD087 | LM87 | 3 | Ir | — | 0 |
| BD088 | LM88 | 3 | Ir | — | 0 |
| BD089 | LM89 | 3 | Ir | — | 0 |
| BD090 | LM90 | 3 | Ir | — | 0 |
| BD091 | LM91 | 3 | Ir | — | 0 |
| BD092 | LM92 | 3 | Ir | — | 0 |
| BD093 | LM93 | 3 | Ir | — | 0 |
| BD094 | LM94 | 3 | Ir | — | 0 |
| BD095 | LM95 | 3 | Ir | — | 0 |
| BD096 | LM96 | 3 | Ir | — | 0 |
| BD097 | LM97 | 3 | Ir | — | 0 |
| BD098 | LM98 | 3 | Ir | — | 0 |
| BD099 | LM99 | 3 | Ir | — | 0 |
| BD100 | LM100 | 3 | Ir | — | 0 |

TABLE 2

| Compound Name | L₁₀₁ | n101 | M₁₀₁ | L₁₀₂ | m101 |
|---|---|---|---|---|---|
| BD101 | LM101 | 3 | Ir | — | 0 |
| BD102 | LM102 | 3 | Ir | — | 0 |
| BD103 | LM103 | 3 | Ir | — | 0 |
| BD104 | LM104 | 3 | Ir | — | 0 |
| BD105 | LM105 | 3 | Ir | — | 0 |
| BD106 | LM106 | 3 | Ir | — | 0 |
| BD107 | LM107 | 3 | Ir | — | 0 |
| BD108 | LM108 | 3 | Ir | — | 0 |
| BD109 | LM109 | 3 | Ir | — | 0 |
| BD110 | LM110 | 3 | Ir | — | 0 |
| BD111 | LM111 | 3 | Ir | — | 0 |
| BD112 | LM112 | 3 | Ir | — | 0 |
| BD113 | LM113 | 3 | Ir | — | 0 |
| BD114 | LM114 | 3 | Ir | — | 0 |
| BD115 | LM115 | 3 | Ir | — | 0 |
| BD116 | LM116 | 3 | Ir | — | 0 |
| BD117 | LM117 | 3 | Ir | — | 0 |
| BD118 | LM118 | 3 | Ir | — | 0 |
| BD119 | LM119 | 3 | Ir | — | 0 |
| BD120 | LM120 | 3 | Ir | — | 0 |
| BD121 | LM121 | 3 | Ir | — | 0 |
| BD122 | LM122 | 3 | Ir | — | 0 |
| BD123 | LM123 | 3 | Ir | — | 0 |
| BD124 | LM124 | 3 | Ir | — | 0 |
| BD125 | LM125 | 3 | Ir | — | 0 |
| BD126 | LM126 | 3 | Ir | — | 0 |
| BD127 | LM127 | 3 | Ir | — | 0 |
| BD128 | LM128 | 3 | Ir | — | 0 |
| BD129 | LM129 | 3 | Ir | — | 0 |
| BD130 | LM130 | 3 | Ir | — | 0 |
| BD131 | LM131 | 3 | Ir | — | 0 |
| BD132 | LM132 | 3 | Ir | — | 0 |
| BD133 | LM133 | 3 | Ir | — | 0 |
| BD134 | LM134 | 3 | Ir | — | 0 |
| BD135 | LM135 | 3 | Ir | — | 0 |
| BD136 | LM136 | 3 | Ir | — | 0 |
| BD137 | LM137 | 3 | Ir | — | 0 |
| BD138 | LM138 | 3 | Ir | — | 0 |
| BD139 | LM139 | 3 | Ir | — | 0 |
| BD140 | LM140 | 3 | Ir | — | 0 |
| BD141 | LM141 | 3 | Ir | — | 0 |
| BD142 | LM142 | 3 | Ir | — | 0 |
| BD143 | LM143 | 3 | Ir | — | 0 |
| BD144 | LM144 | 3 | Ir | — | 0 |
| BD145 | LM145 | 3 | Ir | — | 0 |
| BD146 | LM146 | 3 | Ir | — | 0 |
| BD147 | LM147 | 3 | Ir | — | 0 |
| BD148 | LM148 | 3 | Ir | — | 0 |
| BD149 | LM149 | 3 | Ir | — | 0 |
| BD150 | LM150 | 3 | Ir | — | 0 |
| BD151 | LM151 | 3 | Ir | — | 0 |
| BD152 | LM152 | 3 | Ir | — | 0 |
| BD153 | LM153 | 3 | Ir | — | 0 |
| BD154 | LM154 | 3 | Ir | — | 0 |
| BD155 | LM155 | 3 | Ir | — | 0 |
| BD156 | LM156 | 3 | Ir | — | 0 |
| BD157 | LM157 | 3 | Ir | — | 0 |
| BD158 | LM158 | 3 | Ir | — | 0 |
| BD159 | LM159 | 3 | Ir | — | 0 |
| BD160 | LM160 | 3 | Ir | — | 0 |
| BD161 | LM161 | 3 | Ir | — | 0 |
| BD162 | LM162 | 3 | Ir | — | 0 |
| BD163 | LM163 | 3 | Ir | — | 0 |
| BD164 | LM164 | 3 | Ir | — | 0 |
| BD165 | LM165 | 3 | Ir | — | 0 |
| BD166 | LM166 | 3 | Ir | — | 0 |
| BD167 | LM167 | 3 | Ir | — | 0 |
| BD168 | LM168 | 3 | Ir | — | 0 |
| BD169 | LM169 | 3 | Ir | — | 0 |
| BD170 | LM170 | 3 | Ir | — | 0 |
| BD171 | LM171 | 3 | Ir | — | 0 |
| BD172 | LM172 | 3 | Ir | — | 0 |
| BD173 | LM173 | 3 | Ir | — | 0 |
| BD174 | LM174 | 3 | Ir | — | 0 |
| BD175 | LM175 | 3 | Ir | — | 0 |
| BD176 | LM176 | 3 | Ir | — | 0 |
| BD177 | LM177 | 3 | Ir | — | 0 |
| BD178 | LM178 | 3 | Ir | — | 0 |
| BD179 | LM179 | 3 | Ir | — | 0 |
| BD180 | LM180 | 3 | Ir | — | 0 |
| BD181 | LM181 | 3 | Ir | — | 0 |
| BD182 | LM182 | 3 | Ir | — | 0 |
| BD183 | LM183 | 3 | Ir | — | 0 |
| BD184 | LM184 | 3 | Ir | — | 0 |
| BD185 | LM185 | 3 | Ir | — | 0 |
| BD186 | LM186 | 3 | Ir | — | 0 |
| BD187 | LM187 | 3 | Ir | — | 0 |
| BD188 | LM188 | 3 | Ir | — | 0 |
| BD189 | LM189 | 3 | Ir | — | 0 |
| BD190 | LM190 | 3 | Ir | — | 0 |
| BD191 | LM191 | 3 | Ir | — | 0 |
| BD192 | LM192 | 3 | Ir | — | 0 |
| BD193 | LM193 | 3 | Ir | — | 0 |
| BD194 | LM194 | 3 | Ir | — | 0 |
| BD195 | LM195 | 3 | Ir | — | 0 |
| BD196 | LM196 | 3 | Ir | — | 0 |
| BD197 | LM197 | 3 | Ir | — | 0 |
| BD198 | LM198 | 3 | Ir | — | 0 |
| BD199 | LM199 | 3 | Ir | — | 0 |
| BD200 | LM200 | 3 | Ir | — | 0 |

TABLE 3

| Compound Name | L₁₀₁ | n101 | M₁₀₁ | L₁₀₂ | m101 |
|---|---|---|---|---|---|
| BD201 | LM201 | 3 | Ir | — | 0 |
| BD202 | LM202 | 3 | Ir | — | 0 |
| BD203 | LM203 | 3 | Ir | — | 0 |
| BD204 | LM204 | 3 | Ir | — | 0 |
| BD205 | LM205 | 3 | Ir | — | 0 |
| BD206 | LM206 | 3 | Ir | — | 0 |
| BD207 | LM207 | 3 | Ir | — | 0 |
| BD208 | LM208 | 3 | Ir | — | 0 |
| BD209 | LM209 | 3 | Ir | — | 0 |
| BD210 | LM210 | 3 | Ir | — | 0 |
| BD211 | LM211 | 3 | Ir | — | 0 |
| BD212 | LM212 | 3 | Ir | — | 0 |
| BD213 | LM213 | 3 | Ir | — | 0 |
| BD214 | LM214 | 3 | Ir | — | 0 |
| BD215 | LM215 | 3 | Ir | — | 0 |
| BD216 | LM216 | 3 | Ir | — | 0 |
| BD217 | LM217 | 3 | Ir | — | 0 |
| BD218 | LM218 | 3 | Ir | — | 0 |
| BD219 | LM219 | 3 | Ir | — | 0 |
| BD220 | LM220 | 3 | Ir | — | 0 |
| BD221 | LM221 | 3 | Ir | — | 0 |
| BD222 | LM222 | 3 | Ir | — | 0 |
| BD223 | LM223 | 3 | Ir | — | 0 |
| BD224 | LM224 | 3 | Ir | — | 0 |
| BD225 | LM225 | 3 | Ir | — | 0 |
| BD226 | LM226 | 3 | Ir | — | 0 |
| BD227 | LM227 | 3 | Ir | — | 0 |
| BD228 | LM228 | 3 | Ir | — | 0 |
| BD229 | LM229 | 3 | Ir | — | 0 |
| BD230 | LM230 | 3 | Ir | — | 0 |
| BD231 | LM231 | 3 | Ir | — | 0 |
| BD232 | LM232 | 3 | Ir | — | 0 |
| BD233 | LM233 | 3 | Ir | — | 0 |
| BD234 | LM234 | 3 | Ir | — | 0 |
| BD235 | LM235 | 3 | Ir | — | 0 |
| BD236 | LM236 | 3 | Ir | — | 0 |
| BD237 | LM237 | 3 | Ir | — | 0 |
| BD238 | LM238 | 3 | Ir | — | 0 |
| BD239 | LM239 | 3 | Ir | — | 0 |
| BD240 | LM240 | 3 | Ir | — | 0 |
| BD241 | LM241 | 3 | Ir | — | 0 |
| BD242 | LM242 | 3 | Ir | — | 0 |
| BD243 | LM243 | 3 | Ir | — | 0 |
| BD244 | LFM1 | 3 | Ir | — | 0 |
| BD245 | LFM2 | 3 | Ir | — | 0 |
| BD246 | LFM3 | 3 | Ir | — | 0 |
| BD247 | LFM4 | 3 | Ir | — | 0 |
| BD248 | LFM5 | 3 | Ir | — | 0 |
| BD249 | LFM6 | 3 | Ir | — | 0 |
| BD250 | LFM7 | 3 | Ir | — | 0 |
| BD251 | LFP1 | 3 | Ir | — | 0 |
| BD252 | LFP2 | 3 | Ir | — | 0 |
| BD253 | LFP3 | 3 | Ir | — | 0 |
| BD254 | LFP4 | 3 | Ir | — | 0 |
| BD255 | LFP5 | 3 | Ir | — | 0 |
| BD256 | LFP6 | 3 | Ir | — | 0 |
| BD257 | LFP7 | 3 | Ir | — | 0 |
| BD258 | LM47 | 2 | Ir | AN1 | 1 |
| BD259 | LM47 | 2 | Ir | AN2 | 1 |
| BD260 | LM47 | 2 | Ir | AN3 | 1 |
| BD261 | LM47 | 2 | Ir | AN4 | 1 |
| BD262 | LM47 | 2 | Ir | AN5 | 1 |
| BD263 | LM11 | 2 | Pt | — | 0 |
| BD264 | LM13 | 2 | Pt | — | 0 |
| BD265 | LM15 | 2 | Pt | — | 0 |
| BD266 | LM45 | 2 | Pt | — | 0 |
| BD267 | LM47 | 2 | Pt | — | 0 |
| BD268 | LM49 | 2 | Pt | — | 0 |
| BD269 | LM98 | 2 | Pt | — | 0 |
| BD270 | LM100 | 2 | Pt | — | 0 |
| BD271 | LM102 | 2 | Pt | — | 0 |
| BD272 | LM132 | 2 | Pt | — | 0 |
| BD273 | LM134 | 2 | Pt | — | 0 |
| BD274 | LM136 | 2 | Pt | — | 0 |
| BD275 | LM151 | 2 | Pt | — | 0 |
| BD276 | LM153 | 2 | Pt | — | 0 |
| BD277 | LM158 | 2 | Pt | — | 0 |
| BD278 | LM180 | 2 | Pt | — | 0 |
| BD279 | LM182 | 2 | Pt | — | 0 |
| BD280 | LM187 | 2 | Pt | — | 0 |
| BD281 | LM201 | 2 | Pt | — | 0 |
| BD282 | LM206 | 2 | Pt | — | 0 |
| BD283 | LM211 | 2 | Pt | — | 0 |
| BD284 | LM233 | 2 | Pt | — | 0 |
| BD285 | LM235 | 2 | Pt | — | 0 |
| BD286 | LM240 | 2 | Pt | — | 0 |
| BD287 | LFM5 | 2 | Pt | — | 0 |
| BD288 | LFM6 | 2 | Pt | — | 0 |
| BD289 | LFM7 | 2 | Pt | — | 0 |
| BD290 | LFP5 | 2 | Pt | — | 0 |
| BD291 | LFP6 | 2 | Pt | — | 0 |
| BD292 | LFP7 | 2 | Pt | — | 0 |
| BD293 | LM47 | 1 | Pt | AN1 | 1 |
| BD294 | LM47 | 1 | Pt | AN2 | 1 |
| BD295 | LM47 | 1 | Pt | AN3 | 1 |
| BD296 | LM47 | 1 | Pt | AN4 | 1 |
| BD297 | LM47 | 1 | Pt | AN5 | 1 |

LM1 to LM243, LFM1 to LFM7, and LFP1 to LFP7 in Tables 1 to 3 may be understood by referring to Formulae 1-1 to 1-3 and Tables 4 to 6:

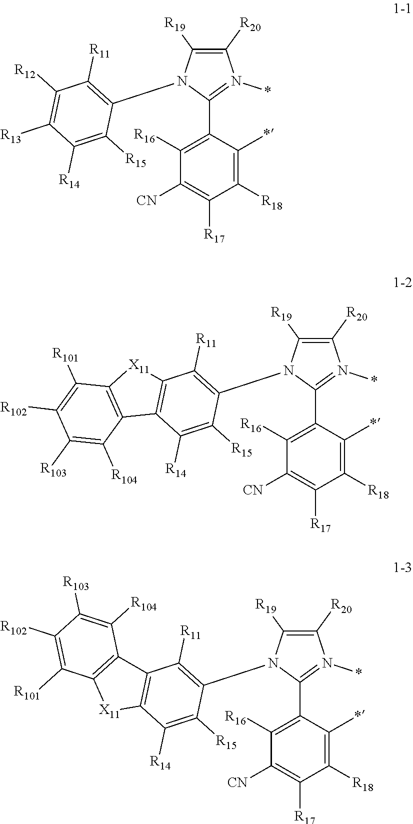

TABLE 4
Formula 1-1

| Ligand name | R₁₁ | R₁₂ | R₁₃ | R₁₄ | R₁₅ | R₁₆ | R₁₇ | R₁₈ | R₁₉ | R₂₀ |
|---|---|---|---|---|---|---|---|---|---|---|
| LM1 | X1 | H | X3 | H | X1 | H | H | H | H | D |
| LM2 | X1 | H | X3 | H | X1 | H | H | H | D | H |
| LM3 | X1 | H | X3 | H | X1 | H | H | H | D | D |
| LM4 | Y1 | H | X3 | H | Y1 | H | H | H | D | D |
| LM5 | Y2 | H | X3 | H | Y2 | H | H | H | D | D |
| LM6 | Y3 | H | X3 | H | Y3 | H | H | H | D | D |
| LM7 | Y3 | D | X3 | D | Y3 | H | H | H | D | D |
| LM8 | Y3 | D | X3 | D | Y3 | D | H | H | D | D |
| LM9 | Y3 | D | X3 | D | Y3 | D | D | H | D | D |
| LM10 | Y3 | D | X3 | D | Y3 | D | D | D | D | D |
| LM11 | Y3 | D | Y11 | D | Y3 | D | D | D | D | D |
| LM12 | Y3 | D | Y11 | D | Y3 | H | X1 | H | D | D |
| LM13 | Y3 | D | Y11 | D | Y3 | D | Y3 | D | D | D |
| LM14 | Y3 | D | Y11 | D | Y3 | H | X4 | H | D | D |
| LM15 | Y3 | D | Y11 | D | Y3 | D | Y12 | D | D | D |
| LM16 | X2 | H | X3 | H | X2 | H | H | H | H | D |
| LM17 | X2 | H | X3 | H | X2 | H | H | H | D | H |
| LM18 | X2 | H | X3 | H | X2 | H | H | H | D | D |
| LM19 | Y4 | H | X3 | H | Y4 | H | H | H | D | D |
| LM20 | Y5 | H | X3 | H | Y5 | H | H | H | D | D |
| LM21 | Y6 | H | X3 | H | Y6 | H | H | H | D | D |
| LM22 | Y7 | H | X3 | H | Y7 | H | H | H | D | D |
| LM23 | Y8 | H | X3 | H | Y8 | H | H | H | D | D |
| LM24 | Y9 | H | X3 | H | Y9 | H | H | H | D | D |
| LM25 | Y10 | H | X3 | H | Y10 | H | H | H | D | D |
| LM26 | Y10 | D | X3 | D | Y10 | H | H | H | D | D |
| LM27 | Y10 | D | X3 | D | Y10 | D | H | H | D | D |
| LM28 | Y10 | D | X3 | D | Y10 | D | D | H | D | D |
| LM29 | Y10 | D | X3 | D | Y10 | D | D | D | D | D |
| LM30 | Y10 | D | Y11 | D | Y10 | D | D | D | D | D |
| LM31 | Y10 | D | Y11 | D | Y10 | H | X1 | H | D | D |
| LM32 | Y10 | D | Y11 | D | Y10 | D | Y3 | D | D | D |
| LM33 | Y10 | D | Y11 | D | Y10 | H | X4 | H | D | D |
| LM34 | Y10 | D | Y11 | D | Y10 | D | Y12 | D | D | D |
| LM35 | X1 | H | X4 | H | X1 | H | H | H | H | D |
| LM36 | X1 | H | X4 | H | X1 | H | H | H | D | H |
| LM37 | X1 | H | X4 | H | X1 | H | H | H | D | D |
| LM38 | Y1 | H | X4 | H | Y1 | H | H | H | D | D |
| LM39 | Y2 | H | X4 | H | Y2 | H | H | H | D | D |
| LM40 | Y3 | H | X4 | H | Y3 | H | H | H | D | D |
| LM41 | Y3 | D | X4 | D | Y3 | H | H | H | D | D |
| LM42 | Y3 | D | X4 | D | Y3 | D | H | H | D | D |
| LM43 | Y3 | D | X4 | D | Y3 | D | D | H | D | D |
| LM44 | Y3 | D | X4 | D | Y3 | D | D | D | D | D |
| LM45 | Y3 | D | Y12 | D | Y3 | D | D | D | D | D |
| LM46 | Y3 | D | Y12 | D | Y3 | H | X1 | H | D | D |
| LM47 | Y3 | D | Y12 | D | Y3 | D | Y3 | D | D | D |
| LM48 | Y3 | D | Y12 | D | Y3 | H | X4 | H | D | D |
| LM49 | Y3 | D | Y12 | D | Y3 | D | Y12 | D | D | D |
| LM50 | X2 | H | X4 | H | X2 | H | H | H | H | D |
| LM51 | X2 | H | X4 | H | X2 | H | H | H | D | H |
| LM52 | X2 | H | X4 | H | X2 | H | H | H | D | D |
| LM53 | Y4 | H | X4 | H | Y4 | H | H | H | D | D |
| LM54 | Y5 | H | X4 | H | Y5 | H | H | H | D | D |
| LM55 | Y6 | H | X4 | H | Y6 | H | H | H | D | D |
| LM56 | Y7 | H | X4 | H | Y7 | H | H | H | D | D |
| LM57 | Y8 | H | X4 | H | Y8 | H | H | H | D | D |
| LM58 | Y9 | H | X4 | H | Y9 | H | H | H | D | D |
| LM59 | Y10 | H | X4 | H | Y10 | H | H | H | D | D |
| LM60 | Y10 | D | X4 | D | Y10 | H | H | H | D | D |
| LM61 | Y10 | D | X4 | D | Y10 | D | H | H | D | D |
| LM62 | Y10 | D | X4 | D | Y10 | D | D | H | D | D |
| LM63 | Y10 | D | X4 | D | Y10 | D | D | D | D | D |
| LM64 | Y10 | D | Y12 | D | Y10 | D | D | D | D | D |
| LM65 | Y10 | D | Y12 | D | Y10 | H | X1 | H | D | D |
| LM66 | Y10 | D | Y12 | D | Y10 | D | Y3 | D | D | D |
| LM67 | Y10 | D | Y12 | D | Y10 | H | X4 | H | D | D |
| LM68 | Y10 | D | Y12 | D | Y10 | D | Y12 | D | D | D |
| LM69 | X1 | H | X5 | H | X1 | H | H | H | H | D |
| LM70 | X1 | H | X5 | H | X1 | H | H | H | D | H |
| LM71 | X1 | H | X5 | H | X1 | H | H | H | D | D |
| LM72 | Y1 | H | X5 | H | Y1 | H | H | H | D | D |
| LM73 | Y2 | H | X5 | H | Y2 | H | H | H | D | D |
| LM74 | Y3 | H | X5 | H | Y3 | H | H | H | D | D |
| LM75 | Y3 | D | X5 | D | Y3 | H | H | H | D | D |
| LM76 | Y3 | D | X5 | D | Y3 | D | H | H | D | D |
| LM77 | Y3 | D | X5 | D | Y3 | D | D | H | D | D |
| LM78 | Y3 | D | X5 | D | Y3 | D | D | D | D | D |
| LM79 | Y3 | D | Y13 | D | Y3 | D | D | D | D | D |
| LM80 | Y3 | D | Y13 | D | Y3 | H | X1 | H | D | D |
| LM81 | Y3 | D | Y13 | D | Y3 | D | Y3 | D | D | D |
| LM82 | Y3 | D | Y13 | D | Y3 | H | X4 | H | D | D |
| LM83 | Y3 | D | Y13 | D | Y3 | D | Y12 | D | D | D |
| LM84 | X2 | H | X5 | H | X2 | H | H | H | H | D |
| LM85 | X2 | H | X5 | H | X2 | H | H | H | D | H |
| LM86 | X2 | H | X5 | H | X2 | H | H | H | D | D |
| LM87 | Y4 | H | X5 | H | Y4 | H | H | H | D | D |
| LM88 | Y5 | H | X5 | H | Y5 | H | H | H | D | D |
| LM89 | Y6 | H | X5 | H | Y6 | H | H | H | D | D |
| LM90 | Y7 | H | X5 | H | Y7 | H | H | H | D | D |
| LM91 | Y8 | H | X5 | H | Y8 | H | H | H | D | D |
| LM92 | Y9 | H | X5 | H | Y9 | H | H | H | D | D |
| LM93 | Y10 | H | X5 | H | Y10 | H | H | H | D | D |
| LM94 | Y10 | D | X5 | D | Y10 | H | H | H | D | D |
| LM95 | Y10 | D | X5 | D | Y10 | D | H | H | D | D |
| LM96 | Y10 | D | X5 | D | Y10 | D | D | H | D | D |
| LM97 | Y10 | D | X5 | D | Y10 | D | D | D | D | D |
| LM98 | Y10 | D | Y13 | D | Y10 | D | D | D | D | D |
| LM99 | Y10 | D | Y13 | D | Y10 | H | X1 | H | D | D |
| LM100 | Y10 | D | Y13 | D | Y10 | D | Y3 | D | D | D |
| LM101 | Y10 | D | Y13 | D | Y10 | H | X4 | H | D | D |
| LM102 | Y10 | D | Y13 | D | Y10 | D | Y12 | D | D | D |
| LM103 | X1 | H | X6 | H | X1 | H | H | H | H | D |
| LM104 | X1 | H | X6 | H | X1 | H | H | H | D | H |
| LM105 | X1 | H | X6 | H | X1 | H | H | H | D | D |
| LM106 | Y1 | H | X6 | H | Y1 | H | H | H | D | D |
| LM107 | Y2 | H | X6 | H | Y2 | H | H | H | D | D |
| LM108 | Y3 | H | X6 | H | Y3 | H | H | H | D | D |
| LM109 | Y3 | D | X6 | D | Y3 | H | H | H | D | D |
| LM110 | Y3 | D | X6 | D | Y3 | D | H | H | D | D |
| LM111 | Y3 | D | X6 | D | Y3 | D | D | H | D | D |
| LM112 | Y3 | D | X6 | D | Y3 | D | D | D | D | D |
| LM113 | Y3 | D | Y14 | D | Y3 | D | D | D | D | D |
| LM114 | Y3 | D | Y14 | D | Y3 | H | X1 | H | D | D |
| LM115 | Y3 | D | Y14 | D | Y3 | D | Y3 | D | D | D |
| LM116 | Y3 | D | Y14 | D | Y3 | H | X4 | H | D | D |
| LM117 | Y3 | D | Y14 | D | Y3 | D | Y12 | D | D | D |
| LM118 | X2 | H | X6 | H | X2 | H | H | H | H | D |
| LM119 | X2 | H | X6 | H | X2 | H | H | H | D | H |
| LM120 | X2 | H | X6 | H | X2 | H | H | H | D | D |
| LM121 | Y4 | H | X6 | H | Y4 | H | H | H | D | D |
| LM122 | Y5 | H | X6 | H | Y5 | H | H | H | D | D |
| LM123 | Y6 | H | X6 | H | Y6 | H | H | H | D | D |
| LM124 | Y7 | H | X6 | H | Y7 | H | H | H | D | D |
| LM125 | Y8 | H | X6 | H | Y8 | H | H | H | D | D |
| LM126 | Y9 | H | X6 | H | Y9 | H | H | H | D | D |
| LM127 | Y10 | H | X6 | H | Y10 | H | H | H | D | D |
| LM128 | Y10 | D | X6 | D | Y10 | H | H | H | D | D |
| LM129 | Y10 | D | X6 | D | Y10 | D | H | H | D | D |
| LM130 | Y10 | D | X6 | D | Y10 | D | D | H | D | D |
| LM131 | Y10 | D | X6 | D | Y10 | D | D | D | D | D |
| LM132 | Y10 | D | Y14 | D | Y10 | D | D | D | D | D |
| LM133 | Y10 | D | Y14 | D | Y10 | H | X1 | H | D | D |
| LM134 | Y10 | D | Y14 | D | Y10 | D | Y3 | D | D | D |
| LM135 | Y10 | D | Y14 | D | Y10 | H | X4 | H | D | D |
| LM136 | Y10 | D | Y14 | D | Y10 | D | Y12 | D | D | D |
| LM137 | X1 | H | X7 | H | X1 | H | H | H | H | D |
| LM138 | X1 | H | X7 | H | X1 | H | H | H | D | H |
| LM139 | X1 | H | X7 | H | X1 | H | H | H | D | D |
| LM140 | Y1 | H | X7 | H | Y1 | H | H | H | D | D |
| LM141 | Y2 | H | X7 | H | Y2 | H | H | H | D | D |
| LM142 | Y3 | H | X7 | H | Y3 | H | H | H | D | D |
| LM143 | Y3 | D | X7 | D | Y3 | H | H | H | D | D |
| LM144 | Y3 | D | X7 | D | Y3 | D | H | H | D | D |
| LM145 | Y3 | D | X7 | D | Y3 | D | D | H | D | D |
| LM146 | Y3 | D | X7 | D | Y3 | D | D | D | D | D |
| LM147 | Y3 | D | X8 | D | Y3 | D | D | D | D | D |
| LM148 | Y3 | D | Y16 | D | Y3 | D | D | D | D | D |
| LM149 | Y3 | D | Y17 | D | Y3 | D | D | D | D | D |
| LM150 | Y3 | D | Y18 | D | Y3 | D | D | D | D | D |
| LM151 | Y3 | D | Y15 | D | Y3 | D | D | D | D | D |
| LM152 | Y3 | D | Y15 | D | Y3 | H | X1 | H | D | D |
| LM153 | Y3 | D | Y15 | D | Y3 | D | Y3 | D | D | D |
| LM154 | Y3 | D | Y16 | D | Y3 | D | Y3 | D | D | D |
| LM155 | Y3 | D | Y17 | D | Y3 | D | Y3 | D | D | D |
| LM156 | Y3 | D | Y18 | D | Y3 | D | Y3 | D | D | D |
| LM157 | Y3 | D | Y15 | D | Y3 | H | X4 | H | D | D |
| LM158 | Y3 | D | Y15 | D | Y3 | D | Y12 | D | D | D |
| LM159 | Y3 | D | Y16 | D | Y3 | D | Y12 | D | D | D |
| LM160 | Y3 | D | Y17 | D | Y3 | D | Y12 | D | D | D |
| LM161 | Y3 | D | Y18 | D | Y3 | D | Y12 | D | D | D |
| LM162 | X2 | H | X7 | H | X2 | H | H | H | H | D |
| LM163 | X2 | H | X7 | H | X2 | H | H | H | D | H |
| LM164 | X2 | H | X7 | H | X2 | H | H | H | D | D |
| LM165 | Y4 | H | X7 | H | Y4 | H | H | H | D | D |
| LM166 | Y5 | H | X7 | H | Y5 | H | H | H | D | D |
| LM167 | Y6 | H | X7 | H | Y6 | H | H | H | D | D |
| LM168 | Y7 | H | X7 | H | Y7 | H | H | H | D | D |
| LM169 | Y8 | H | X7 | H | Y8 | H | H | H | D | D |
| LM170 | Y9 | H | X7 | H | Y9 | H | H | H | D | D |
| LM171 | Y10 | H | X7 | H | Y10 | H | H | H | D | D |
| LM172 | Y10 | D | X7 | D | Y10 | H | H | H | D | D |
| LM173 | Y10 | D | X7 | D | Y10 | D | H | H | D | D |
| LM174 | Y10 | D | X7 | D | Y10 | D | D | H | D | D |
| LM175 | Y10 | D | X7 | D | Y10 | D | D | D | D | D |
| LM176 | Y10 | D | X8 | D | Y10 | D | D | D | D | D |
| LM177 | Y10 | D | Y16 | D | Y10 | D | D | D | D | D |
| LM178 | Y10 | D | Y17 | D | Y10 | D | D | D | D | D |
| LM179 | Y10 | D | Y18 | D | Y10 | D | D | D | D | D |
| LM180 | Y10 | D | Y15 | D | Y10 | D | D | D | D | D |
| LM181 | Y10 | D | Y15 | D | Y10 | H | X1 | H | D | D |
| LM182 | Y10 | D | Y15 | D | Y10 | D | Y3 | D | D | D |
| LM183 | Y10 | D | Y16 | D | Y10 | D | Y3 | D | D | D |
| LM184 | Y10 | D | Y17 | D | Y10 | D | Y3 | D | D | D |
| LM185 | Y10 | D | Y18 | D | Y10 | D | Y3 | D | D | D |
| LM186 | Y10 | D | Y15 | D | Y10 | H | X4 | H | D | D |
| LM187 | Y10 | D | Y15 | D | Y10 | D | Y12 | D | D | D |
| LM188 | Y10 | D | Y16 | D | Y10 | D | Y12 | D | D | D |
| LM189 | Y10 | D | Y17 | D | Y10 | D | Y12 | D | D | D |
| LM190 | Y10 | D | Y18 | D | Y10 | D | Y12 | D | D | D |
| LM191 | X1 | X7 | H | H | X1 | H | H | H | H | D |
| LM192 | X1 | X7 | H | H | X1 | H | H | H | D | H |
| LM193 | X1 | X7 | H | H | X1 | H | H | H | D | D |
| LM194 | Y1 | X7 | H | H | Y1 | H | H | H | D | D |
| LM195 | Y2 | X7 | H | H | Y2 | H | H | H | D | D |
| LM196 | Y3 | X7 | H | H | Y3 | H | H | H | D | D |
| LM197 | Y3 | X7 | D | D | Y3 | H | H | H | D | D |
| LM198 | Y3 | X7 | D | D | Y3 | D | H | H | D | D |
| LM199 | Y3 | X7 | D | D | Y3 | D | D | H | D | D |
| LM200 | Y3 | X7 | D | D | Y3 | D | D | D | D | D |
| LM201 | Y3 | Y15 | D | D | Y3 | D | D | D | D | D |
| LM202 | Y3 | Y16 | D | D | Y3 | D | D | D | D | D |
| LM203 | Y3 | Y17 | D | D | Y3 | D | D | D | D | D |
| LM204 | Y3 | Y18 | D | D | Y3 | D | D | D | D | D |
| LM205 | Y3 | Y15 | D | D | Y3 | H | X1 | H | D | D |
| LM206 | Y3 | Y15 | D | D | Y3 | D | Y3 | D | D | D |
| LM207 | Y3 | Y16 | D | D | Y3 | D | Y3 | D | D | D |
| LM208 | Y3 | Y17 | D | D | Y3 | D | Y3 | D | D | D |
| LM209 | Y3 | Y18 | D | D | Y3 | D | Y3 | D | D | D |
| LM210 | Y3 | Y15 | D | D | Y3 | H | X4 | H | D | D |
| LM211 | Y3 | Y15 | D | D | Y3 | D | Y12 | D | D | D |
| LM212 | Y3 | Y16 | D | D | Y3 | D | Y12 | D | D | D |
| LM213 | Y3 | Y17 | D | D | Y3 | D | Y12 | D | D | D |
| LM214 | Y3 | Y18 | D | D | Y3 | D | Y12 | D | D | D |
| LM215 | X2 | X7 | H | H | X2 | H | H | H | H | D |
| LM216 | X2 | X7 | H | H | X2 | H | H | H | D | H |
| LM217 | X2 | X7 | H | H | X2 | H | H | H | D | D |
| LM218 | Y4 | X7 | H | H | Y4 | H | H | H | D | D |
| LM219 | Y5 | X7 | H | H | Y5 | H | H | H | D | D |
| LM220 | Y6 | X7 | H | H | Y6 | H | H | H | D | D |
| LM221 | Y7 | X7 | H | H | Y7 | H | H | H | D | D |
| LM222 | Y8 | X7 | H | H | Y8 | H | H | H | D | D |
| LM223 | Y9 | X7 | H | H | Y9 | H | H | H | D | D |
| LM224 | Y10 | X7 | H | H | Y10 | H | H | H | D | D |
| LM225 | Y10 | X7 | D | D | Y10 | H | H | H | D | D |
| LM226 | Y10 | X7 | D | D | Y10 | D | H | H | D | D |
| LM227 | Y10 | X7 | D | D | Y10 | D | D | H | D | D |
| LM228 | Y10 | X7 | D | D | Y10 | D | D | D | D | D |
| LM229 | Y10 | X8 | D | D | Y10 | D | D | D | D | D |
| LM230 | Y10 | Y16 | D | D | Y10 | D | D | D | D | D |
| LM231 | Y10 | Y17 | D | D | Y10 | D | D | D | D | D |
| LM232 | Y10 | Y18 | D | D | Y10 | D | D | D | D | D |
| LM233 | Y10 | Y15 | D | D | Y10 | D | D | D | D | D |
| LM234 | Y10 | Y15 | D | D | Y10 | H | X1 | H | D | D |
| LM235 | Y10 | Y15 | D | D | Y10 | D | Y3 | D | D | D |
| LM236 | Y10 | Y16 | D | D | Y10 | D | Y3 | D | D | D |
| LM237 | Y10 | Y17 | D | D | Y10 | D | Y3 | D | D | D |
| LM238 | Y10 | Y18 | D | D | Y10 | D | Y3 | D | D | D |
| LM239 | Y10 | Y15 | D | D | Y10 | H | X4 | H | D | D |
| LM240 | Y10 | Y15 | D | D | Y10 | D | Y12 | D | D | D |
| LM241 | Y10 | Y16 | D | D | Y10 | D | Y12 | D | D | D |
| LM242 | Y10 | Y17 | D | D | Y10 | D | Y12 | D | D | D |
| LM243 | Y10 | Y18 | D | D | Y10 | D | Y12 | D | D | D |

TABLE 5
Formula 1-2

| Ligand name | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFM1 | Y10 | N—Ph | D | D | D | D | D | Y10 | D | D | D | D | D |
| LFM2 | Y10 | S | D | D | D | D | D | Y10 | D | D | D | D | D |
| LFM3 | Y10 | O | D | D | D | D | D | Y10 | D | D | D | D | D |
| LFM4 | Y3 | O | D | D | D | D | D | Y3 | D | D | D | D | D |
| LFM5 | Y10 | O | D | D | D | D | D | Y10 | D | D | D | D | D |
| LFM6 | Y10 | O | D | D | D | D | D | Y10 | D | Y3 | D | D | D |
| LFM7 | Y10 | O | D | D | D | D | D | Y10 | D | Y12 | D | D | D |

TABLE 6
Formula 1-3

| Ligand name | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFP1 | Y10 | N—Ph | D | D | D | D | D | Y10 | D | D | D | D | D |
| LFP2 | Y10 | S | D | D | D | D | D | Y10 | D | D | D | D | D |
| LFP3 | Y10 | O | D | D | D | D | D | Y10 | D | D | D | D | D |
| LFP4 | Y3 | O | D | D | D | D | D | Y3 | D | D | D | D | D |
| LFP5 | Y10 | O | D | D | D | D | D | Y10 | D | D | D | D | D |
| LFP6 | Y10 | O | D | D | D | D | D | Y10 | D | Y3 | D | D | D |
| LFP7 | Y10 | O | D | D | D | D | D | Y10 | D | Y12 | D | D | D |

X1 to X10 and Y1 to Y18 in Tables 4 to 6 are the same as described below, and Ph in the tables refers to a phenyl group:

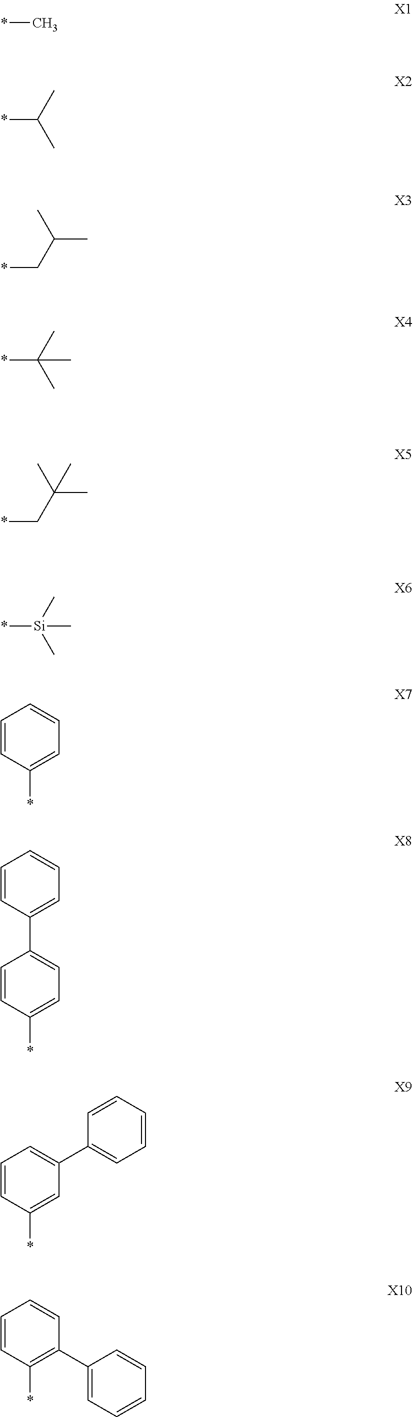

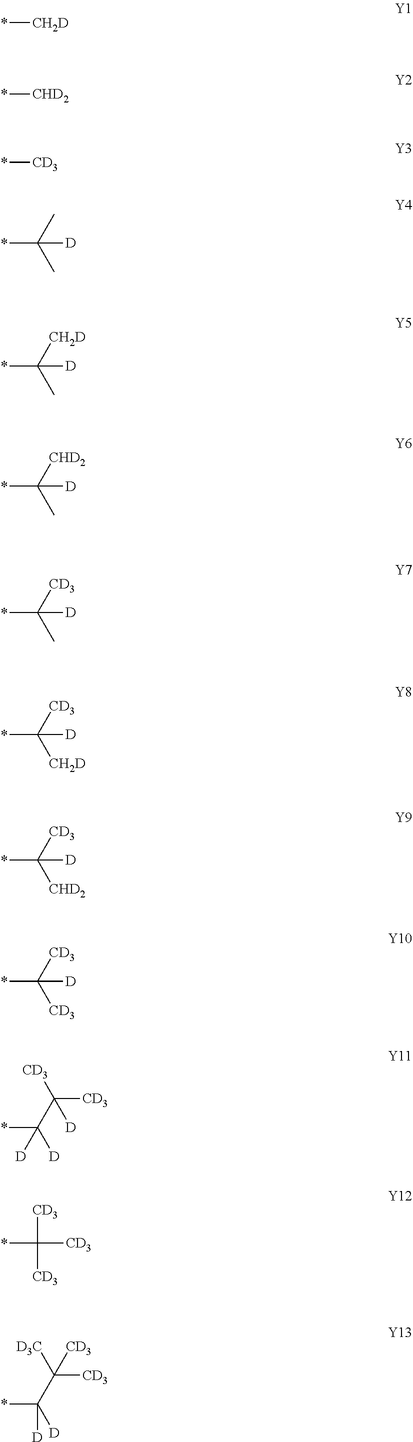

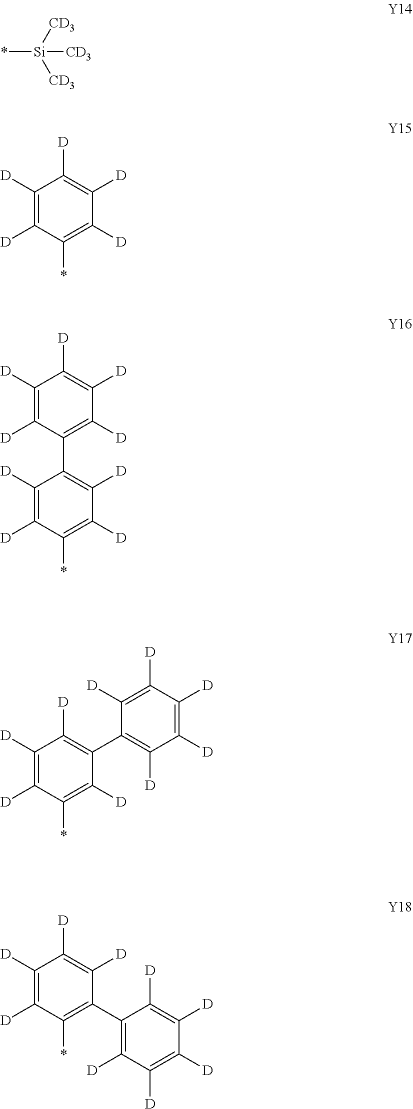 [SMILES: *[Si](C([2H])([2H])[2H])(C([2H])([2H])[2H])C([2H])([2H])[2H] ; *c1c([2H])c([2H])c([2H])c([2H])c1[2H] ; *c1c([2H])c([2H])c(-c2c([2H])c([2H])c([2H])c([2H])c2[2H])c([2H])c1[2H] ; *c1c([2H])c([2H])c([2H])c(-c2c([2H])c([2H])c([2H])c([2H])c2[2H])c1[2H] ; *c1c([2H])c([2H])c([2H])c([2H])c1-c1c([2H])c([2H])c([2H])c([2H])c1[2H]]

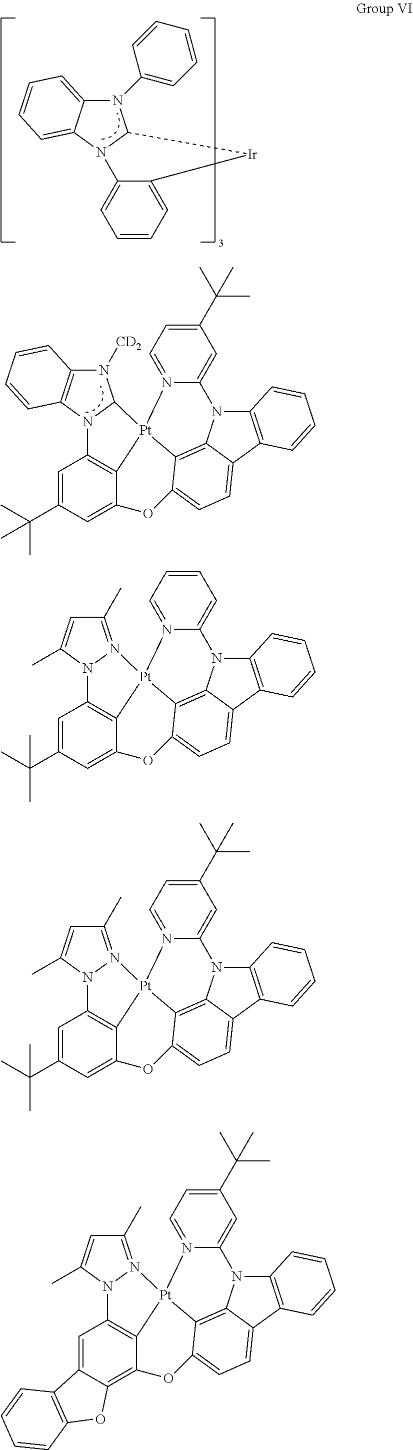

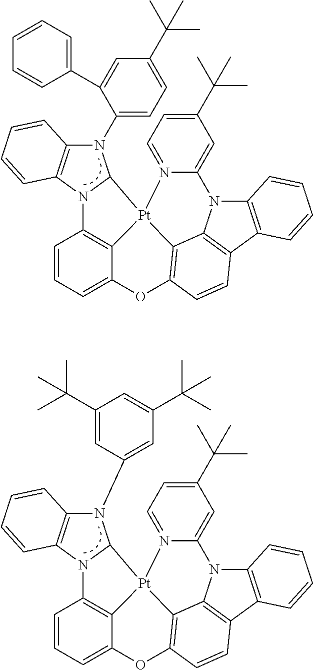

In one or more embodiments, the sensitizer may include the thermally activated delayed fluorescence emitter represented by Formula 201 or 202.

For example, A₂₁₁ in Formula 202 may be a substituted or unsubstituted π electron-deficient nitrogen-free cyclic group.

In one or more embodiments, the π electron-deficient nitrogen-free cyclic group may be a phenyl group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group; or a condensed cyclic group of two or more r electron-deficient nitrogen-free cyclic groups, but embodiments of the present disclosure are not limited thereto.

For example, D₂₁₁ in Formula 202 may be: —F, a cyano group, or an π-electron deficient nitrogen-containing cyclic group;

a C₁-C₆₀ alkyl group, an π-electron deficient nitrogen-containing cyclic group, or an π electron-deficient nitrogen-free cyclic group, each substituted with at least one of —F or a cyano group; or

an π-electron deficient nitrogen-containing cyclic group, substituted with at least one deuterium, a C₁-C₆₀ alkyl group, an π-electron deficient nitrogen-containing cyclic group, or an π electron-deficient nitrogen-free cyclic group.

In one or more embodiments, the π electron-deficient nitrogen-free cyclic group is the same as described herein.

The term “π electron-deficient nitrogen-containing cyclic group” used herein refers to a cyclic group having at least one —N=moiety, and, for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, or a benzimidazolobenzimidazole group; or a condensed cyclic group in which two or more π electron-efficient nitrogen-containing cyclic groups are condensed with each other.

In one or more embodiments, the sensitizer may be one from Groups VII to XIII, but embodiments of the present disclosure are not limited thereto: Group VII

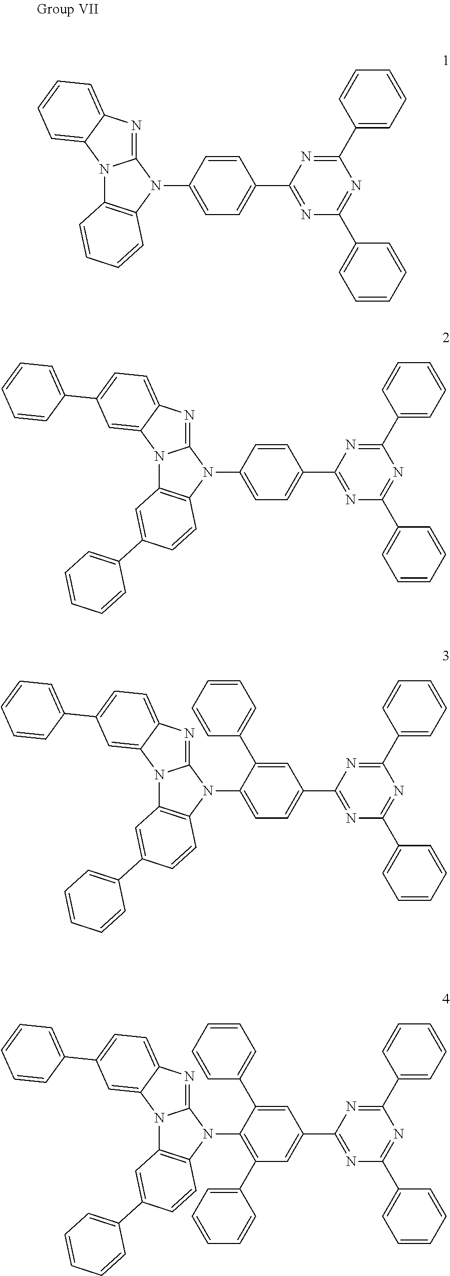 [SMILES: c1ccc(-c2nc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)nc(-c7ccccc7)n6)cc5)c5ccc(-c6ccccc6)cc5n4c3c2)cc1 ; c1ccc(-c2ccc3nc4n(-c5ccc(-c6nc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)nc(-c7ccccc7)n6)cc5-c5ccccc5)c5ccc(-c6ccccc6)cc5n4c3c2)cc1]

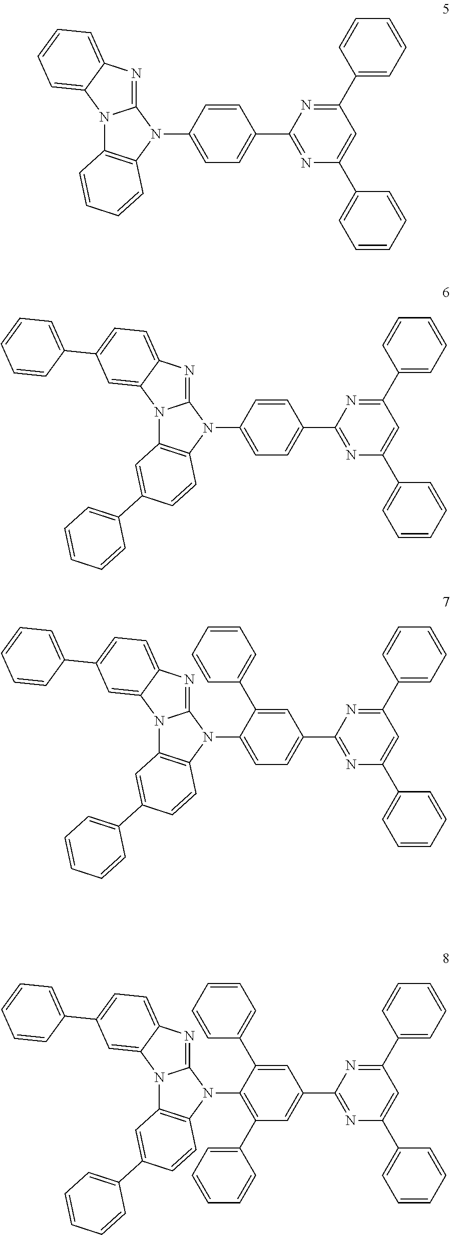 [SMILES: c1ccc(-c2cc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)cc(-c7ccccc7)n6)cc5)c5ccc(-c6ccccc6)cc5n4c3c2)cc1 ; c1ccc(-c2ccc3nc4n(-c5ccc(-c6nc(-c7ccccc7)cc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)cc(-c7ccccc7)n6)cc5-c5ccccc5)c5ccc(-c6ccccc6)cc5n4c3c2)cc1]

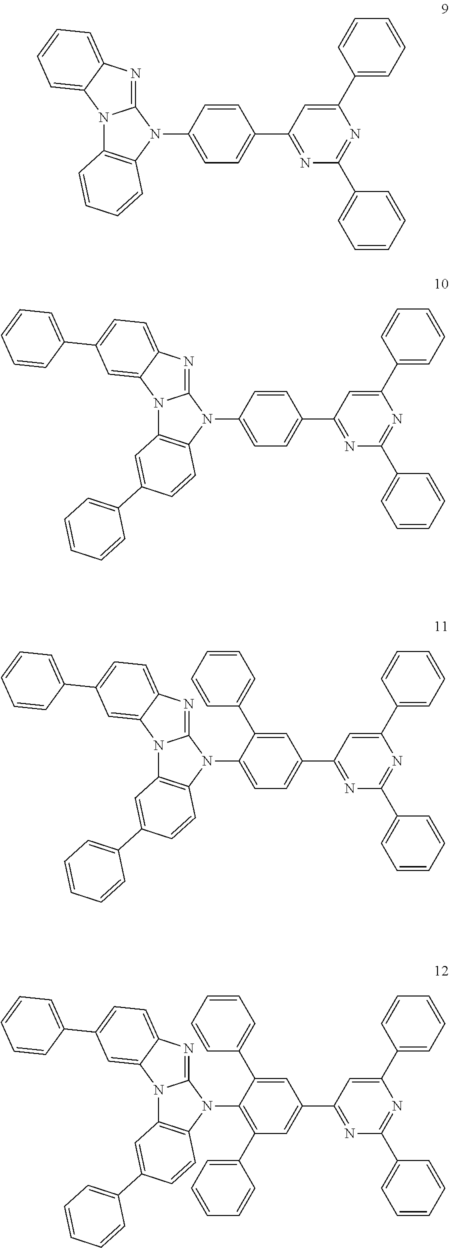 [SMILES: c1ccc(-c2cc(-c3ccc(-n4c5ccccc5n5c6ccccc6nc45)cc3)nc(-c3ccccc3)n2)cc1 ; c1ccc(-c2ccc3nc4n(-c5ccc(-c6cc(-c7ccccc7)nc(-c7ccccc7)n6)cc5)c5ccc(-c6ccccc6)cc5n4c3c2)cc1 ; c1ccc(-c2ccc3nc4n(-c5ccc(-c6cc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6cc(-c7ccccc7)nc(-c7ccccc7)n6)cc5-c5ccccc5)c5ccc(-c6ccccc6)cc5n4c3c2)cc1]

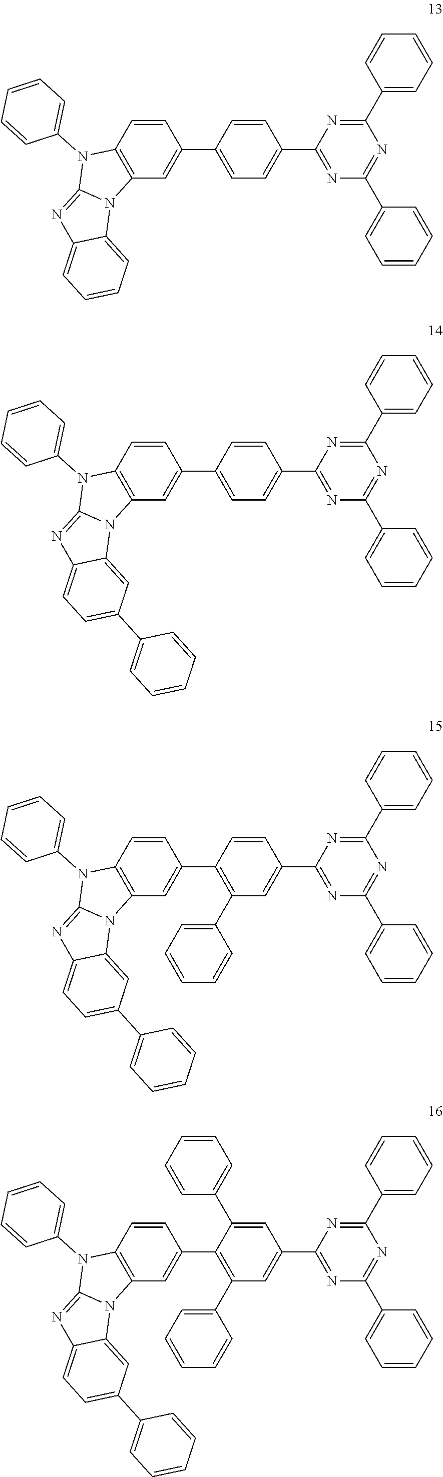 [SMILES: c1ccc(-c2nc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5ccccc5)c5ccc(-c6ccc(-c7nc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)nc(-c8ccccc8)n7)cc6-c6ccccc6)cc5n4c3c2)cc1]

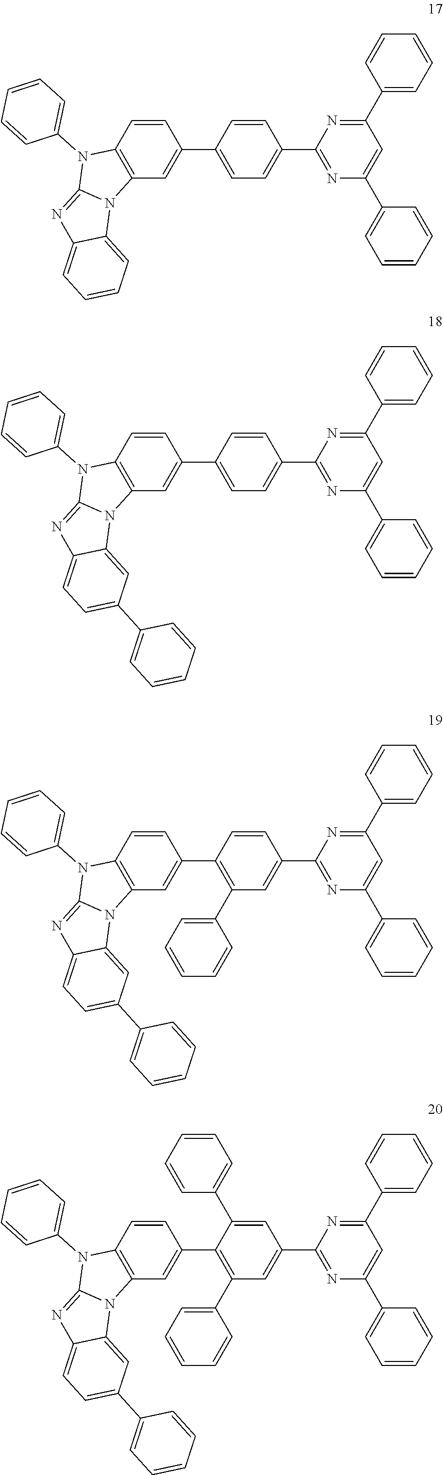 [SMILES: c1ccc(-c2cc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5ccccc5)c5ccc(-c6ccc(-c7nc(-c8ccccc8)cc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)cc(-c8ccccc8)n7)cc6-c6ccccc6)cc5n4c3c2)cc1]

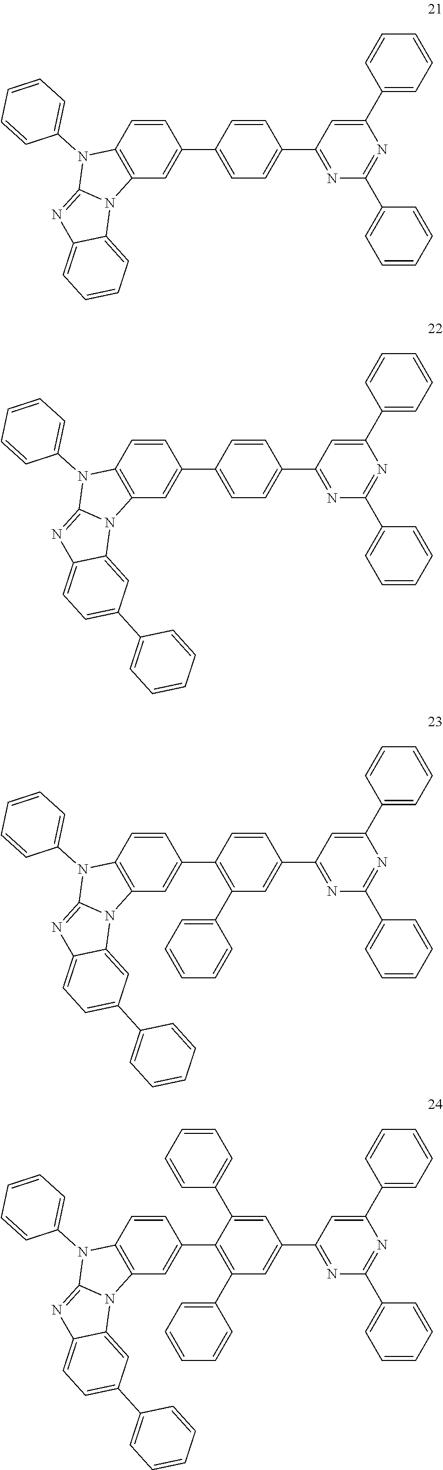 [SMILES: c1ccc(-c2cc(-c3ccc(-c4ccc5c(c4)n4c6ccccc6nc4n5-c4ccccc4)cc3)nc(-c3ccccc3)n2)cc1 ; c1ccc(-c2ccc3nc4n(-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5ccccc5)c5ccc(-c6ccc(-c7cc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7cc(-c8ccccc8)nc(-c8ccccc8)n7)cc6-c6ccccc6)cc5n4c3c2)cc1]

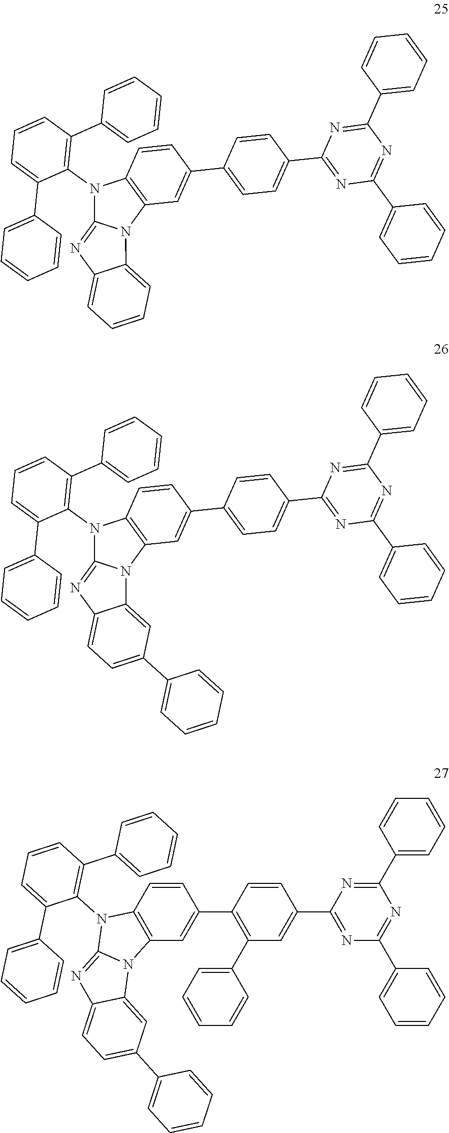 [SMILES: c1ccc(-c2nc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6-c6ccccc6)cc5n4c3c2)cc1]

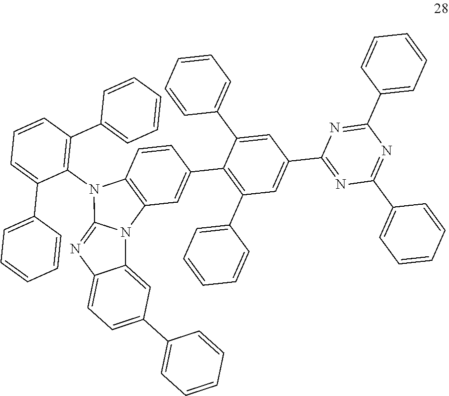 [SMILES: c1ccc(-c2ccc3nc4n(-c5c(-c6ccccc6)cccc5-c5ccccc5)c5ccc(-c6c(-c7ccccc7)cc(-c7nc(-c8ccccc8)nc(-c8ccccc8)n7)cc6-c6ccccc6)cc5n4c3c2)cc1]

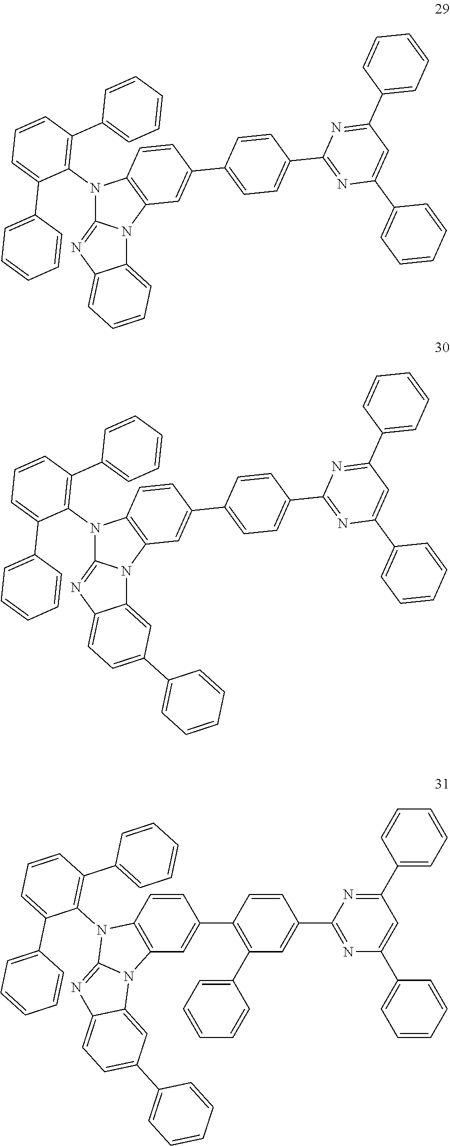 [SMILES: c1ccc(-c2cc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6-c6ccccc6)cc5n4c3c2)cc1]

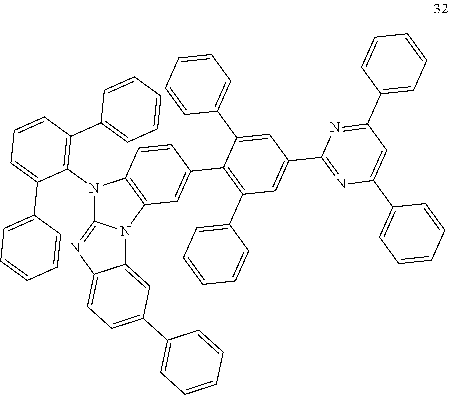 [SMILES: c1ccc(-c2ccc3nc4n(-c5c(-c6ccccc6)cccc5-c5ccccc5)c5ccc(-c6c(-c7ccccc7)cc(-c7nc(-c8ccccc8)cc(-c8ccccc8)n7)cc6-c6ccccc6)cc5n4c3c2)cc1]

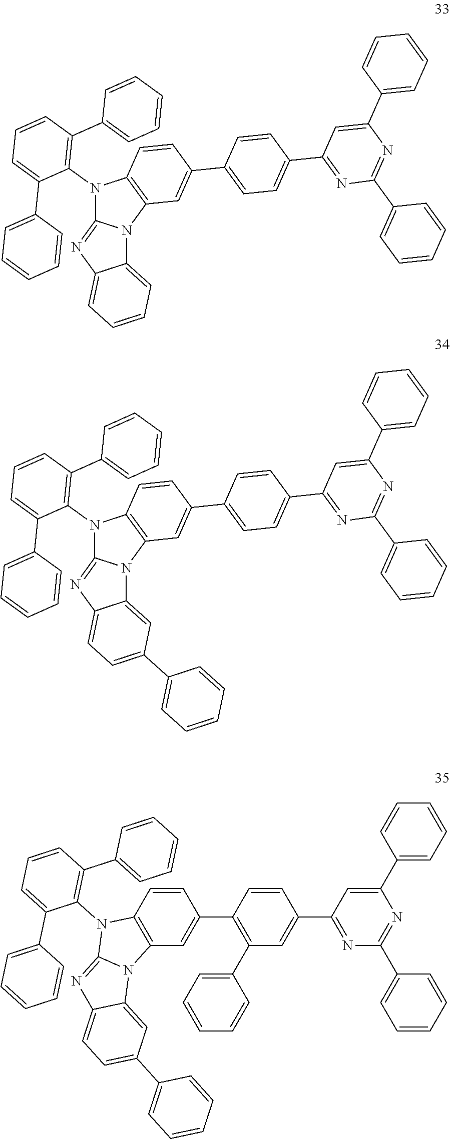 [SMILES: c1ccc(-c2cc(-c3ccc(-c4ccc5c(c4)n4c6ccccc6nc4n5-c4c(-c5ccccc5)cccc4-c4ccccc4)cc3)nc(-c3ccccc3)n2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6-c6ccccc6)cc5n4c3c2)cc1]

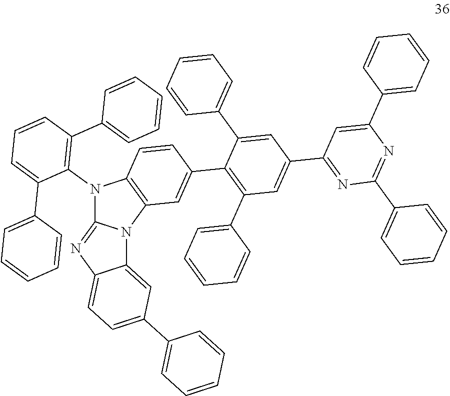 [SMILES: c1ccc(-c2ccc3nc4n(-c5c(-c6ccccc6)cccc5-c5ccccc5)c5ccc(-c6c(-c7ccccc7)cc(-c7cc(-c8ccccc8)nc(-c8ccccc8)n7)cc6-c6ccccc6)cc5n4c3c2)cc1]

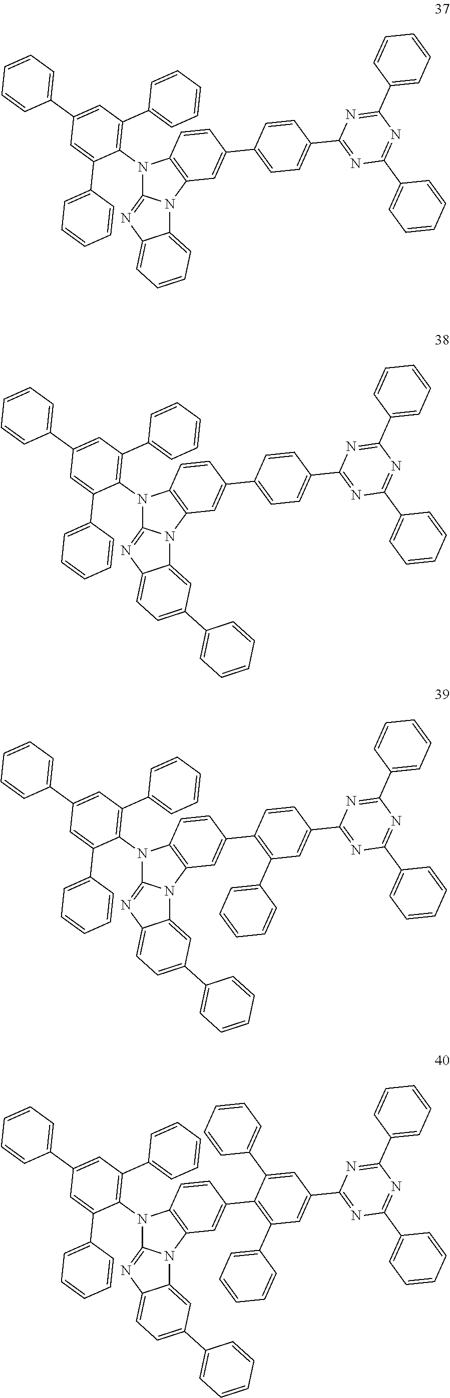

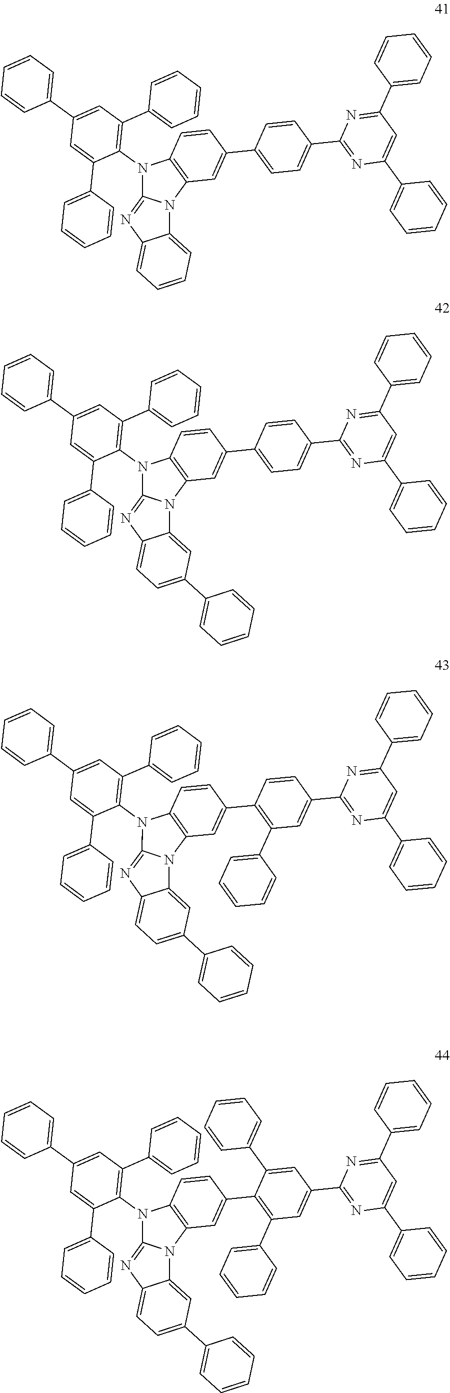

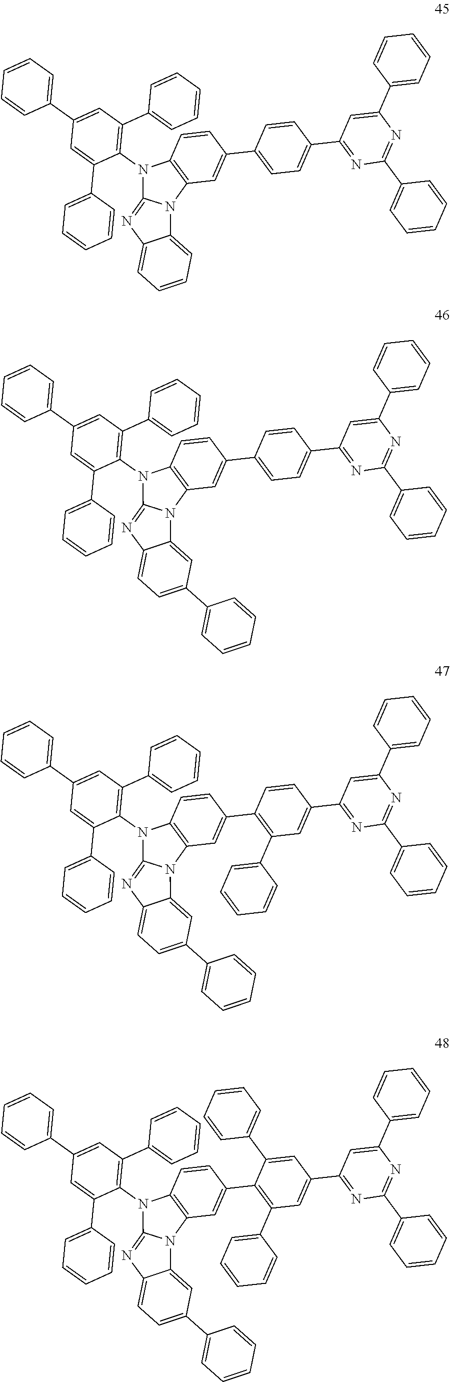

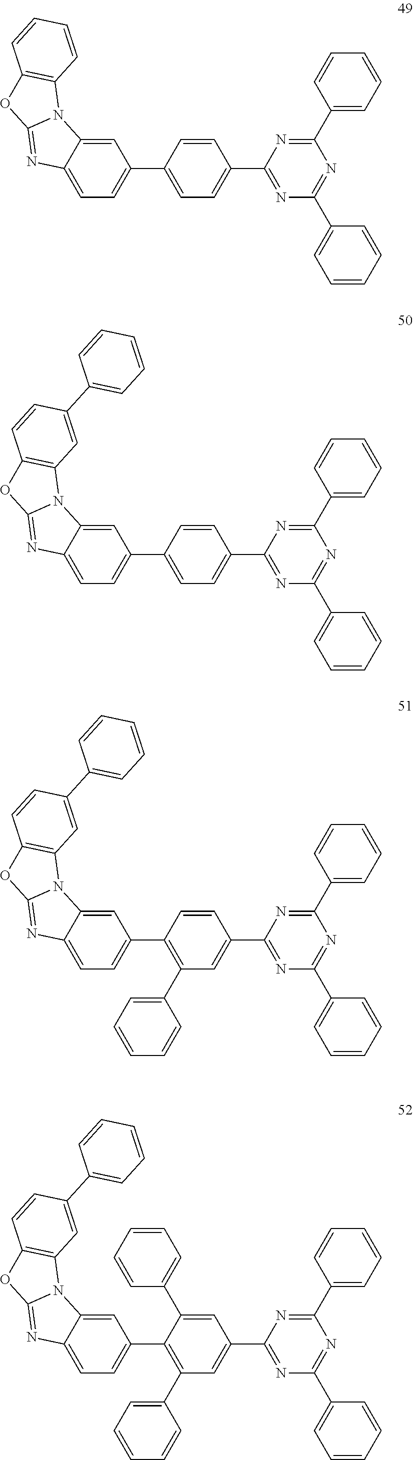 [SMILES: c1ccc(-c2nc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)nc(-c8ccccc8)n7)cc6)cc5n4c3c2)cc1 ; c1ccc(-c2ccc3oc4nc5ccc(-c6ccc(-c7nc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)nc(-c8ccccc8)n7)cc6-c6ccccc6)cc5n4c3c2)cc1]

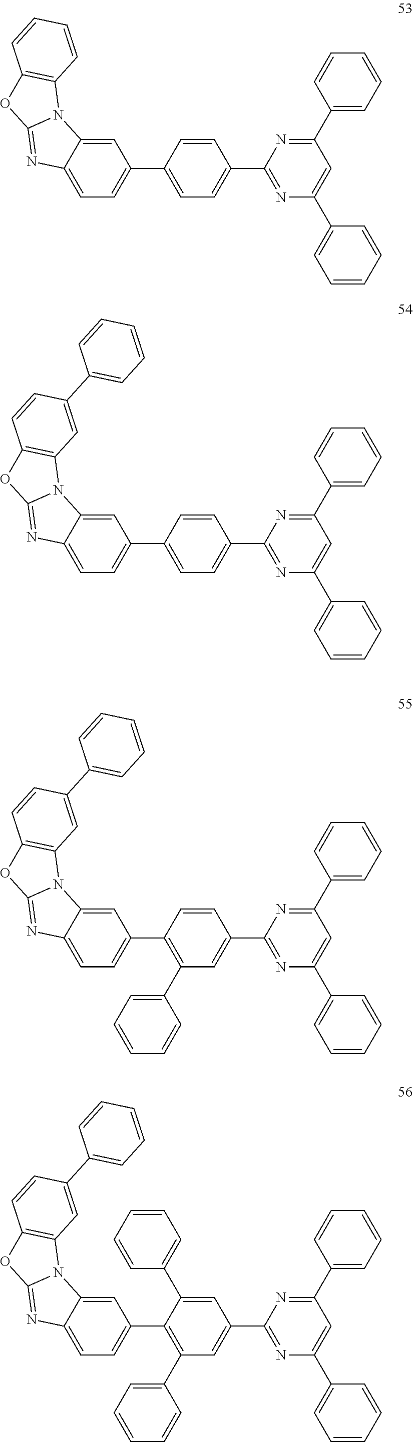 [SMILES: c1ccc(-c2cc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)cc(-c8ccccc8)n7)cc6)cc5n4c3c2)cc1 ; c1ccc(-c2ccc3oc4nc5ccc(-c6ccc(-c7nc(-c8ccccc8)cc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)cc(-c8ccccc8)n7)cc6-c6ccccc6)cc5n4c3c2)cc1]

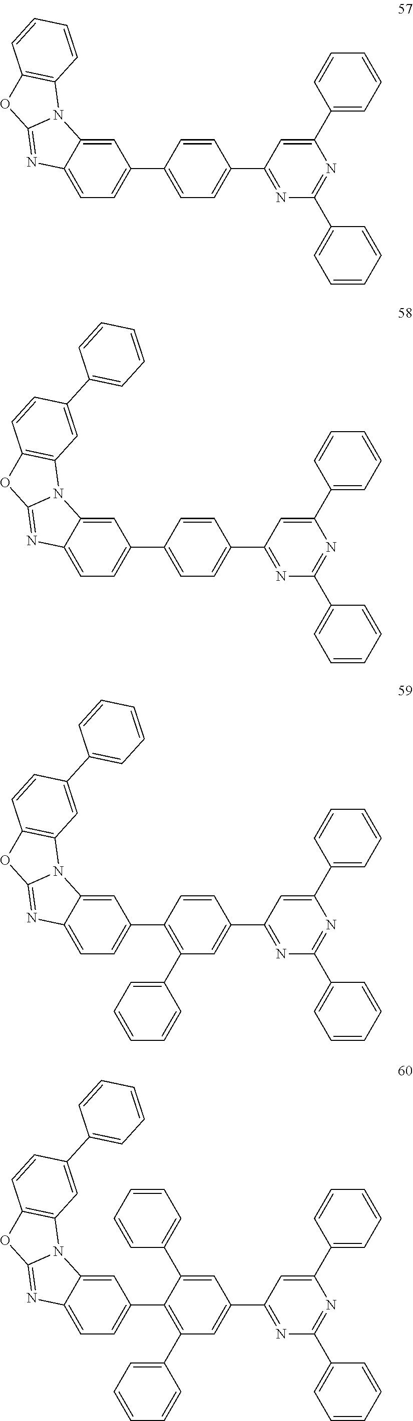 [SMILES: c1ccc(-c2cc(-c3ccc(-c4ccc5nc6oc7ccccc7n6c5c4)cc3)nc(-c3ccccc3)n2)cc1 ; c1ccc(-c2ccc3oc4nc5ccc(-c6ccc(-c7cc(-c8ccccc8)nc(-c8ccccc8)n7)cc6)cc5n4c3c2)cc1 ; c1ccc(-c2ccc3oc4nc5ccc(-c6ccc(-c7cc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7cc(-c8ccccc8)nc(-c8ccccc8)n7)cc6-c6ccccc6)cc5n4c3c2)cc1]

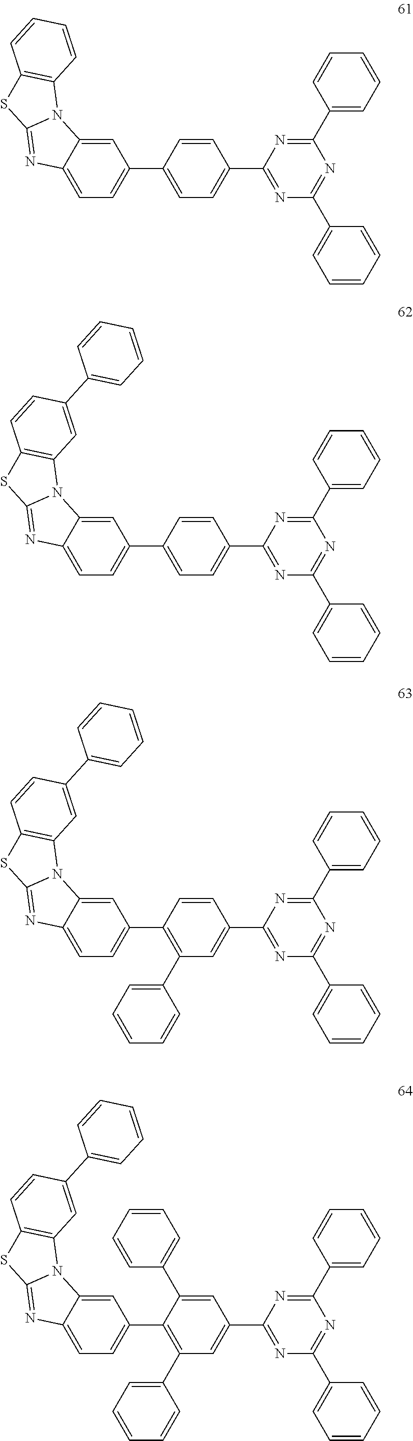 [SMILES: c1ccc(-c2nc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)nc(-c8ccccc8)n7)cc6)cc5n4c3c2)cc1 ; c1ccc(-c2ccc3sc4nc5ccc(-c6ccc(-c7nc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7nc(-c8ccccc8)nc(-c8ccccc8)n7)cc6-c6ccccc6)cc5n4c3c2)cc1]

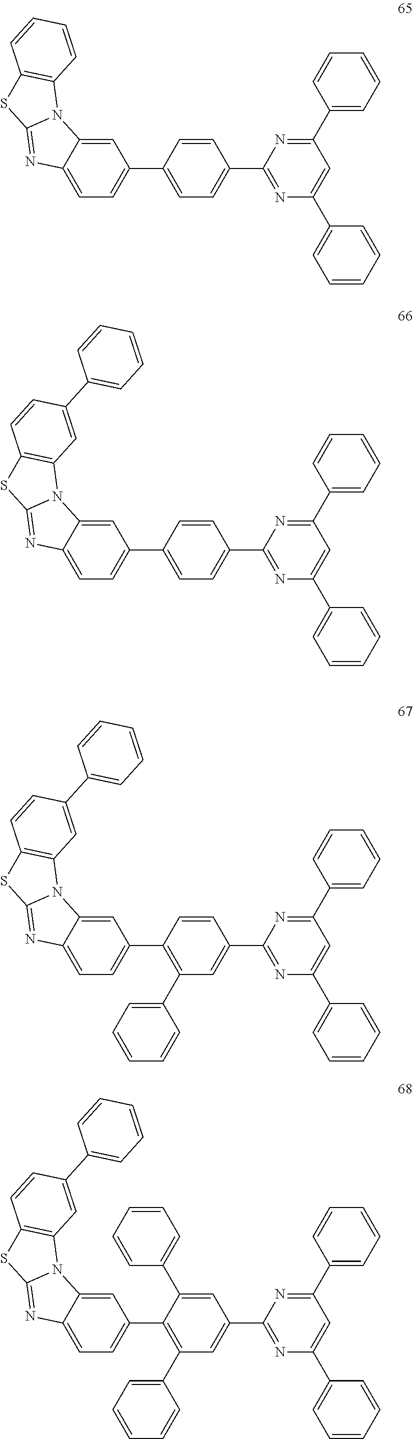

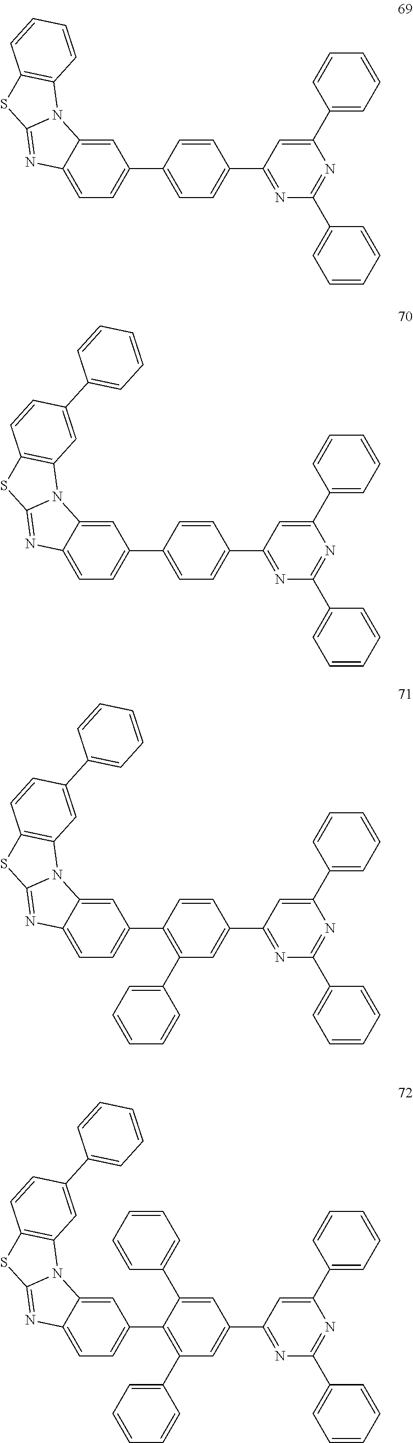 [SMILES: c1ccc(-c2cc(-c3ccc(-c4ccc5nc6sc7ccccc7n6c5c4)cc3)nc(-c3ccccc3)n2)cc1 ; c1ccc(-c2ccc3sc4nc5ccc(-c6ccc(-c7cc(-c8ccccc8)nc(-c8ccccc8)n7)cc6)cc5n4c3c2)cc1 ; c1ccc(-c2ccc3sc4nc5ccc(-c6ccc(-c7cc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7cc(-c8ccccc8)nc(-c8ccccc8)n7)cc6-c6ccccc6)cc5n4c3c2)cc1]

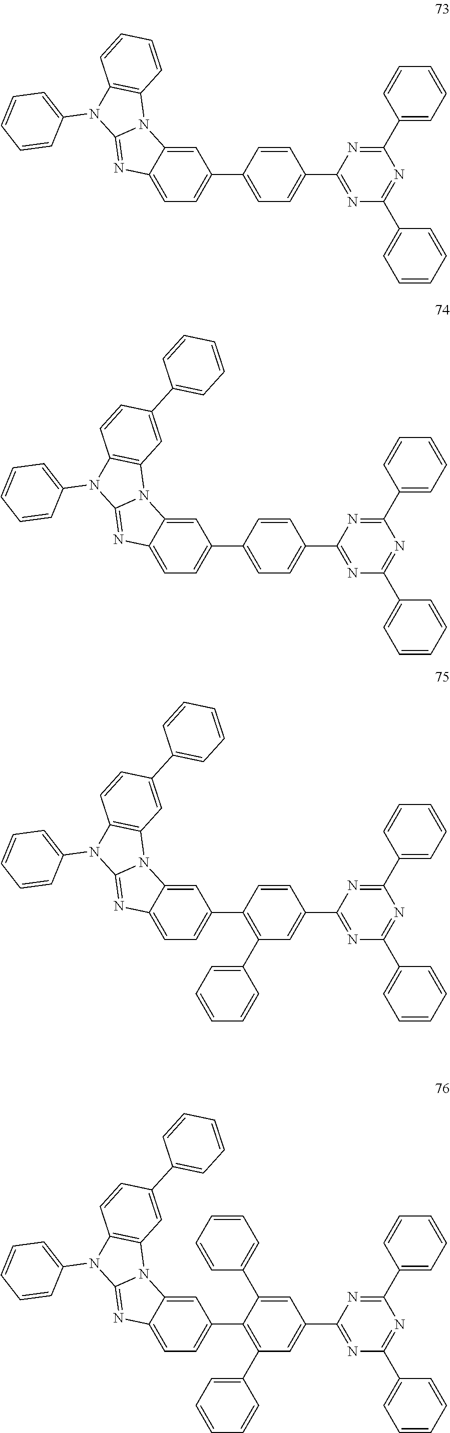 [SMILES: c1ccc(-c2nc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2ccccc2)cc1 ; c1ccc(-c2ccc3c(c2)n2c4cc(-c5ccc(-c6nc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2ccccc2)cc1]

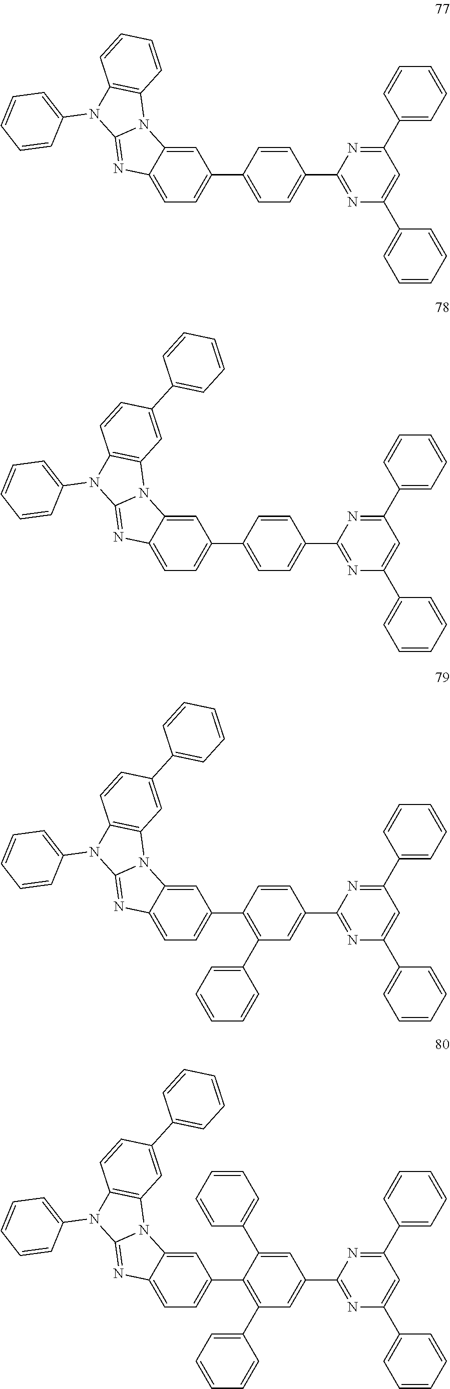 [SMILES: c1ccc(-c2cc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2ccccc2)cc1 ; c1ccc(-c2ccc3c(c2)n2c4cc(-c5ccc(-c6nc(-c7ccccc7)cc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2ccccc2)cc1]

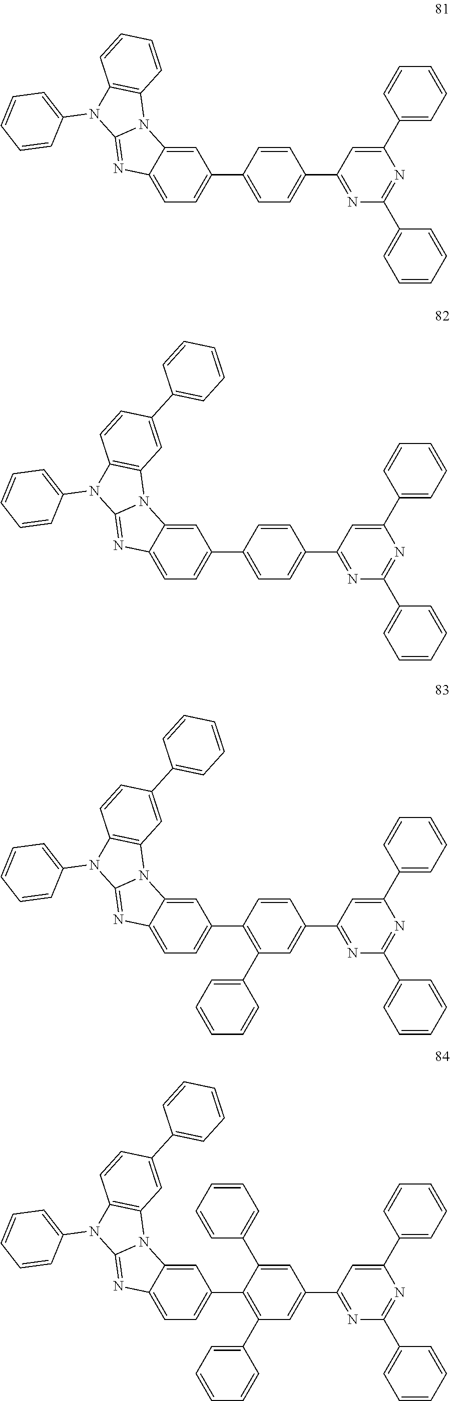 [SMILES: c1ccc(-c2cc(-c3ccc(-c4ccc5nc6n(-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2ccccc2)cc1 ; c1ccc(-c2ccc3c(c2)n2c4cc(-c5ccc(-c6cc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2ccccc2)cc1]

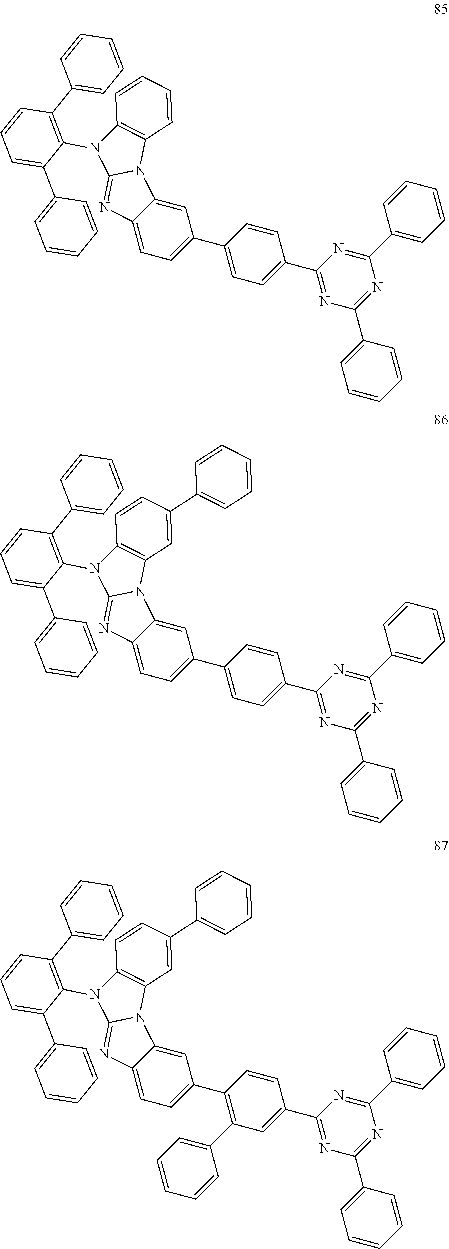 [SMILES: c1ccc(-c2nc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2c(-c3ccccc3)cccc2-c2ccccc2)cc1 ; c1ccc(-c2ccc3c(c2)n2c4cc(-c5ccc(-c6nc(-c7ccccc7)nc(-c7ccccc7)n6)cc5-c5ccccc5)ccc4nc2n3-c2c(-c3ccccc3)cccc2-c2ccccc2)cc1]

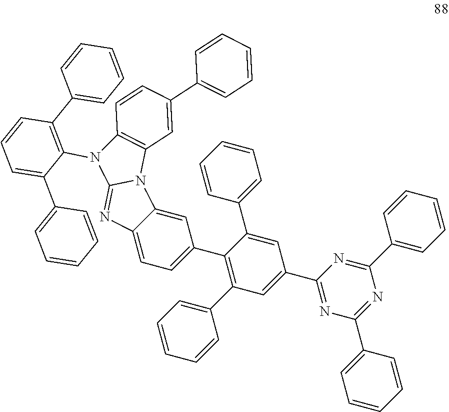 [SMILES: c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2c(-c3ccccc3)cccc2-c2ccccc2)cc1]

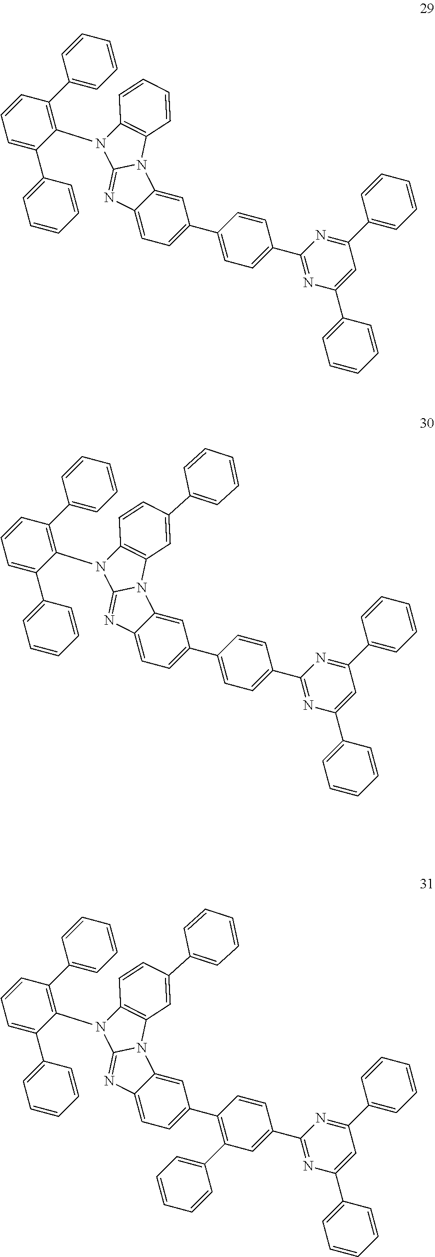 [SMILES: c1ccc(-c2cc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2c(-c3ccccc3)cccc2-c2ccccc2)cc1 ; c1ccc(-c2ccc3c(c2)n2c4cc(-c5ccc(-c6nc(-c7ccccc7)cc(-c7ccccc7)n6)cc5-c5ccccc5)ccc4nc2n3-c2c(-c3ccccc3)cccc2-c2ccccc2)cc1]

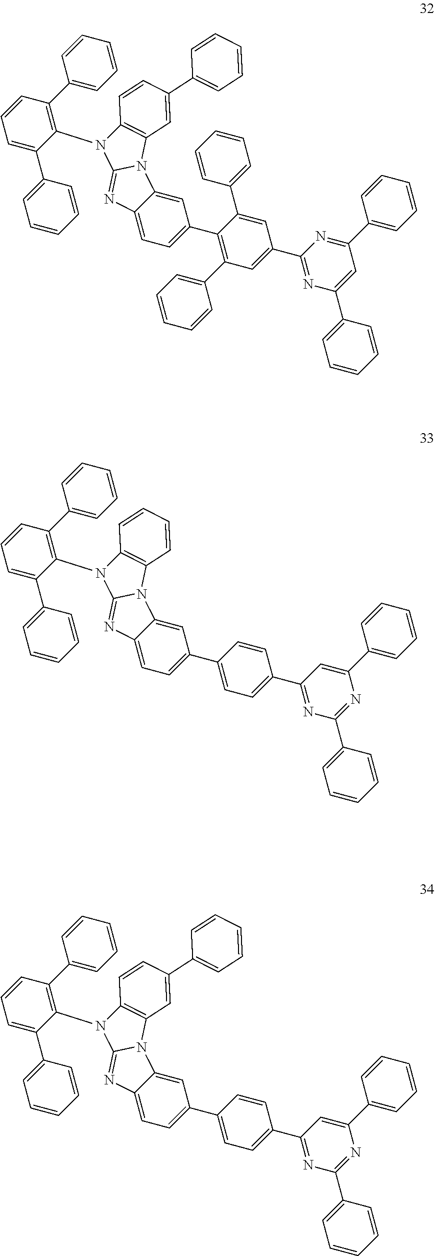 [SMILES: c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2c(-c3ccccc3)cccc2-c2ccccc2)cc1 ; c1ccc(-c2cc(-c3ccc(-c4ccc5nc6n(-c7c(-c8ccccc8)cccc7-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2c(-c3ccccc3)cccc2-c2ccccc2)cc1]

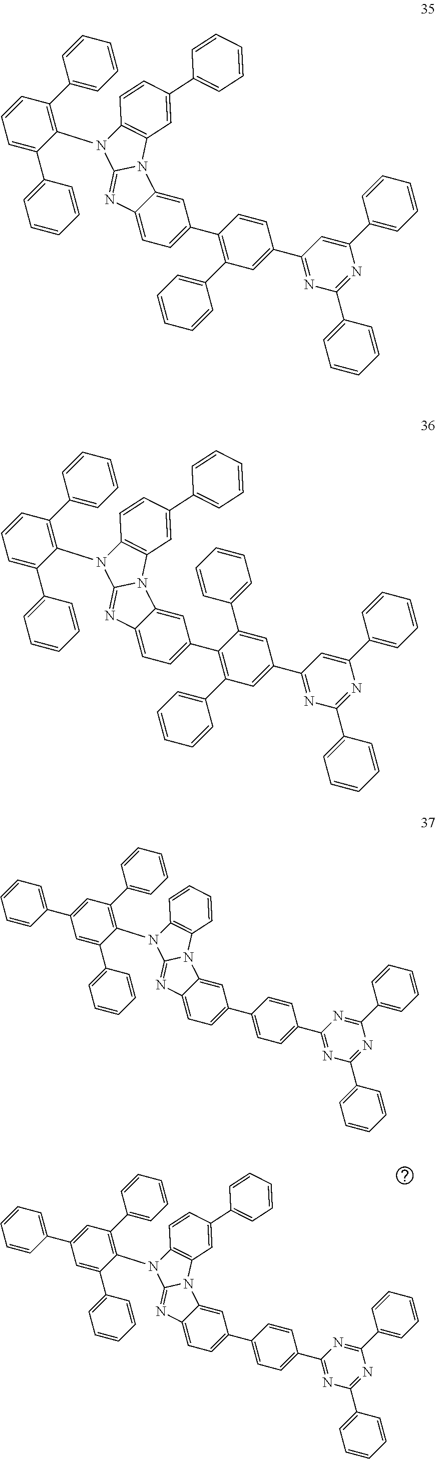 [SMILES: c1ccc(-c2ccc3c(c2)n2c4cc(-c5ccc(-c6cc(-c7ccccc7)nc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2c(-c3ccccc3)cccc2-c2ccccc2)cc1 ; c1ccc(-c2cc(-c3ccccc3)c(-n3c4ccccc4n4c5cc(-c6ccc(-c7nc(-c8ccccc8)nc(-c8ccccc8)n7)cc6)ccc5nc34)c(-c3ccccc3)c2)cc1 ; c1ccc(-c2cc(-c3ccccc3)c(-n3c4ccc(-c5ccccc5)cc4n4c5cc(-c6ccc(-c7nc(-c8ccccc8)nc(-c8ccccc8)n7)cc6)ccc5nc34)c(-c3ccccc3)c2)cc1]

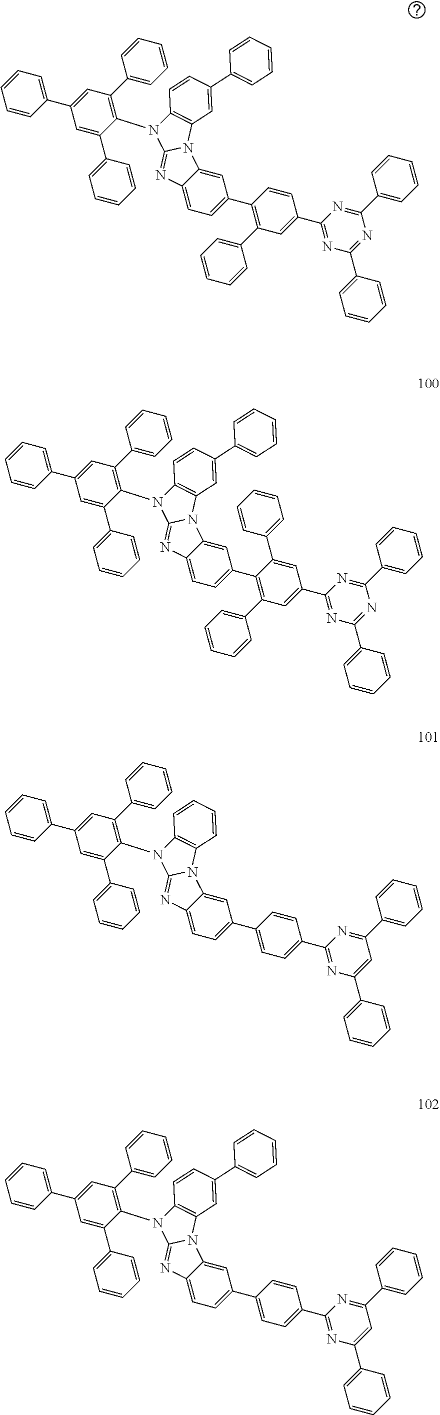

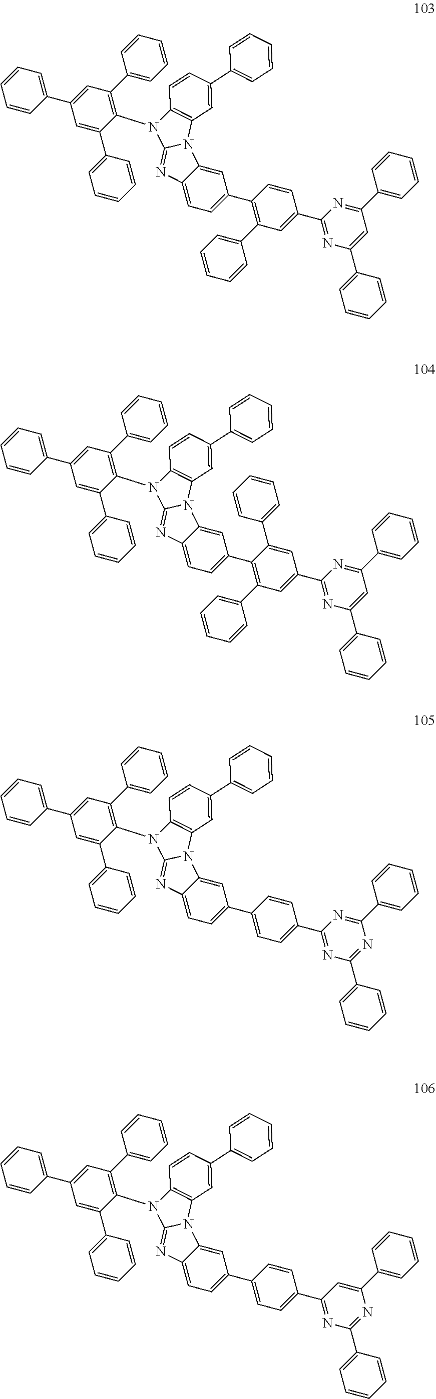

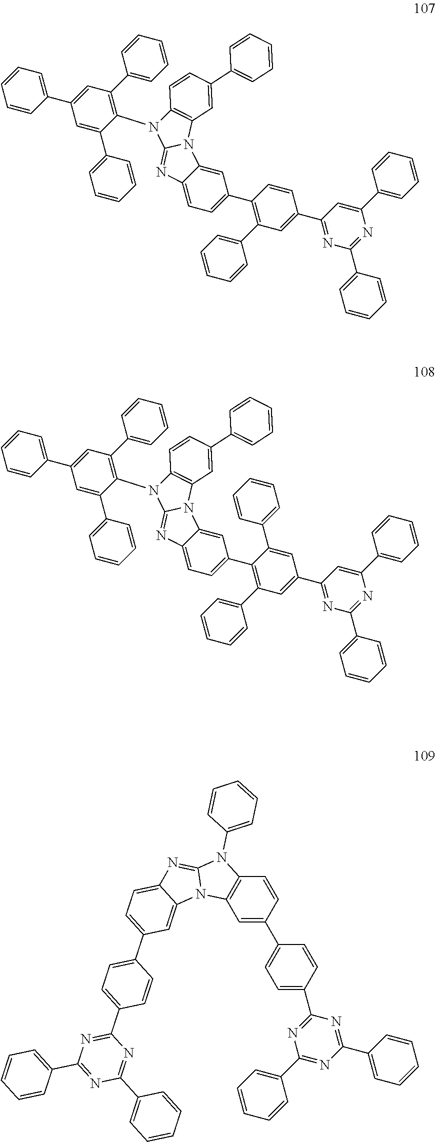 [SMILES: c1ccc(-c2cc(-c3ccccc3)c(-n3c4ccc(-c5ccccc5)cc4n4c5cc(-c6ccc(-c7cc(-c8ccccc8)nc(-c8ccccc8)n7)cc6-c6ccccc6)ccc5nc34)c(-c3ccccc3)c2)cc1 ; c1ccc(-c2cc(-c3ccccc3)c(-n3c4ccc(-c5ccccc5)cc4n4c5cc(-c6c(-c7ccccc7)cc(-c7cc(-c8ccccc8)nc(-c8ccccc8)n7)cc6-c6ccccc6)ccc5nc34)c(-c3ccccc3)c2)cc1 ; c1ccc(-c2nc(-c3ccccc3)nc(-c3ccc(-c4ccc5nc6n(-c7ccccc7)c7ccc(-c8ccc(-c9nc(-c%10ccccc%10)nc(-c%10ccccc%10)n9)cc8)cc7n6c5c4)cc3)n2)cc1]

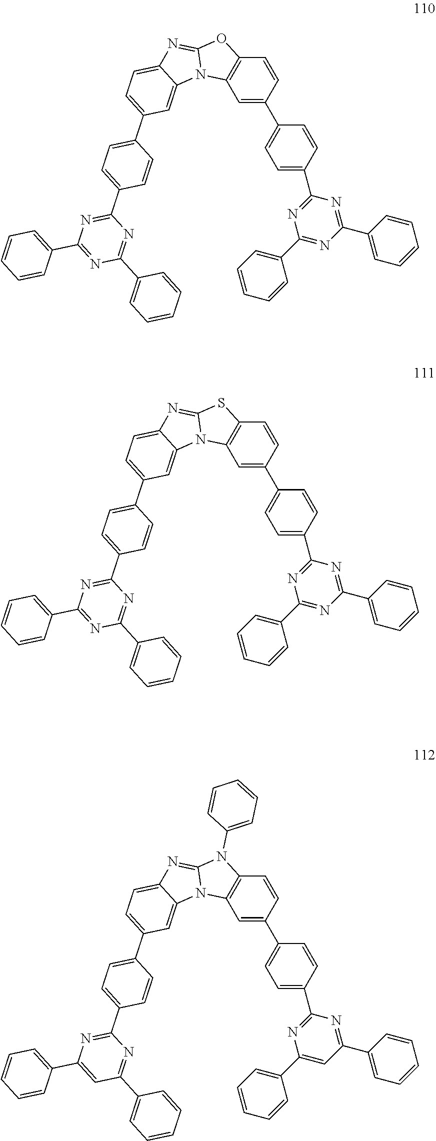 [SMILES: c1ccc(-c2nc(-c3ccccc3)nc(-c3ccc(-c4ccc5nc6oc7ccc(-c8ccc(-c9nc(-c%10ccccc%10)nc(-c%10ccccc%10)n9)cc8)cc7n6c5c4)cc3)n2)cc1 ; c1ccc(-c2nc(-c3ccccc3)nc(-c3ccc(-c4ccc5nc6sc7ccc(-c8ccc(-c9nc(-c%10ccccc%10)nc(-c%10ccccc%10)n9)cc8)cc7n6c5c4)cc3)n2)cc1 ; c1ccc(-c2cc(-c3ccccc3)nc(-c3ccc(-c4ccc5nc6n(-c7ccccc7)c7ccc(-c8ccc(-c9nc(-c%10ccccc%10)cc(-c%10ccccc%10)n9)cc8)cc7n6c5c4)cc3)n2)cc1]

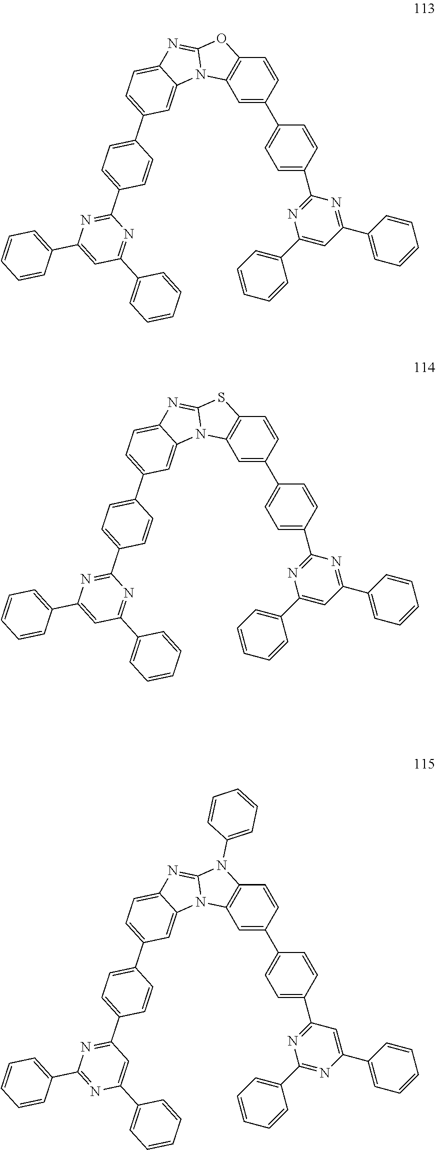 [SMILES: c1ccc(-c2cc(-c3ccccc3)nc(-c3ccc(-c4ccc5nc6oc7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccccc3)nc(-c3ccc(-c4ccc5nc6sc7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccc(-c4ccc5nc6n(-c7ccccc7)c7ccc(-c8ccc(-c9cc(-c%10ccccc%10)nc(-c%10ccccc%10)n9)cc8)cc7n6c5c4)cc3)nc(-c3ccccc3)n2)cc1]

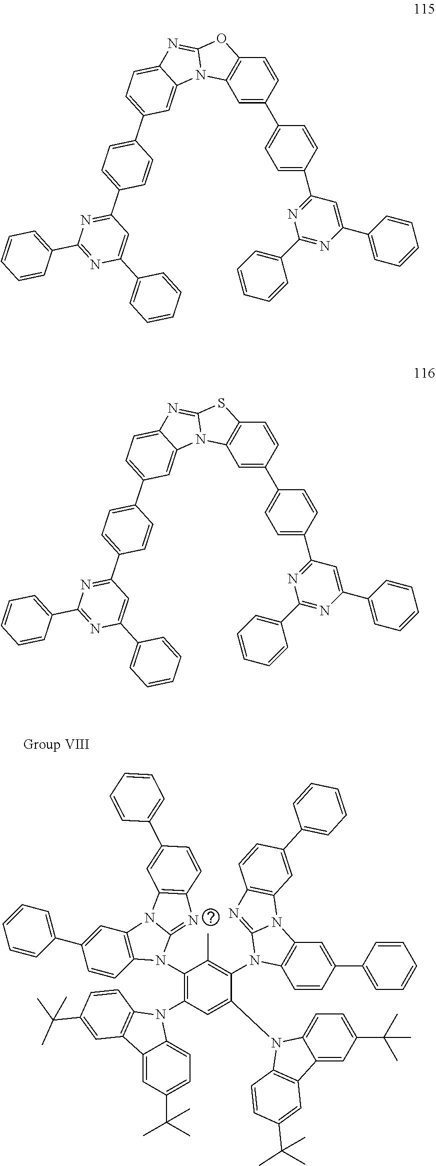 [SMILES: c1ccc(-c2cc(-c3ccc(-c4ccc5nc6oc7ccc(-c8ccc(-c9cc(-c%10ccccc%10)nc(-c%10ccccc%10)n9)cc8)cc7n6c5c4)cc3)nc(-c3ccccc3)n2)cc1 ; c1ccc(-c2cc(-c3ccc(-c4ccc5nc6sc7ccc(-c8ccc(-c9cc(-c%10ccccc%10)nc(-c%10ccccc%10)n9)cc8)cc7n6c5c4)cc3)nc(-c3ccccc3)n2)cc1 ; Cc1c(-n2c3ccc(-c4ccccc4)cc3n3c4cc(-c5ccccc5)ccc4nc23)c(-n2c3ccc(C(C)(C)C)cc3c3cc(C(C)(C)C)ccc32)cc(-n2c3ccc(C(C)(C)C)cc3c3cc(C(C)(C)C)ccc32)c1-n1c2ccc(-c3ccccc3)cc2n2c3cc(-c4ccccc4)ccc3nc12]

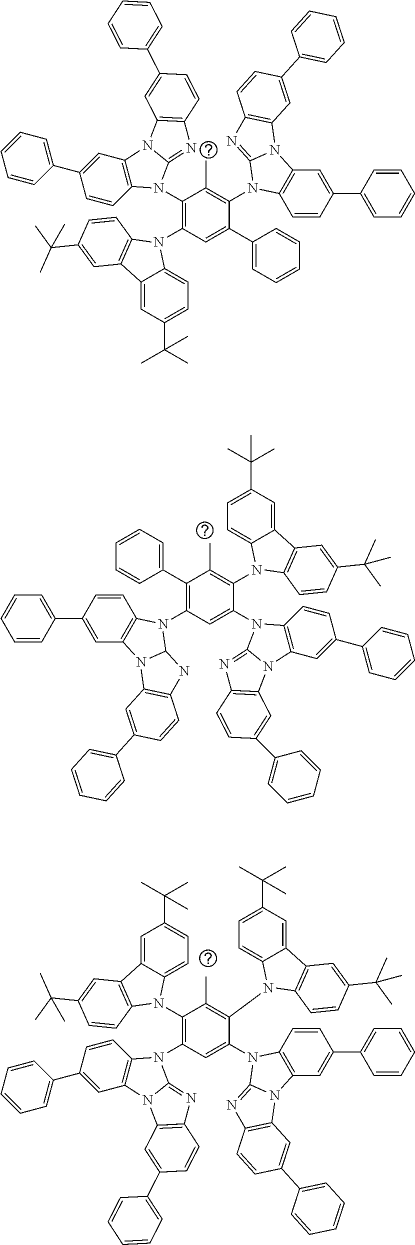

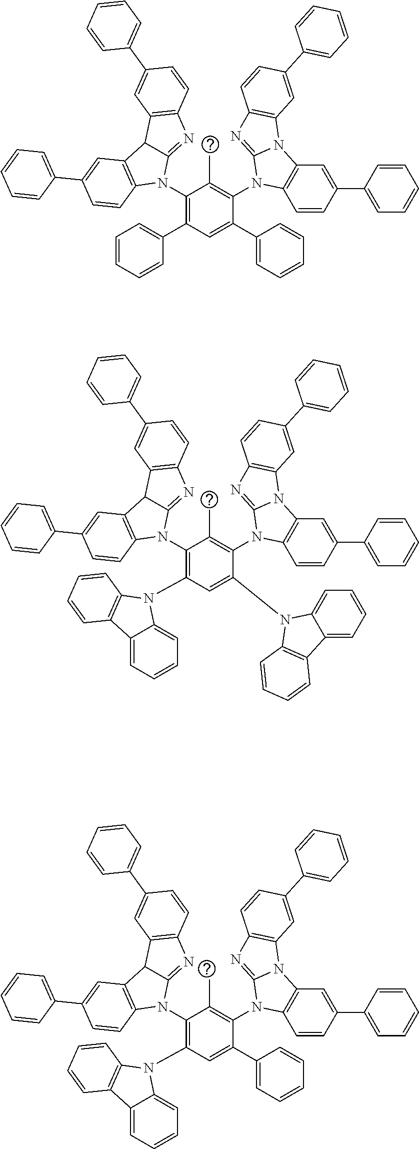 [SMILES: Cc1c(N2C3=Nc4ccc(-c5ccccc5)cc4C3c3cc(-c4ccccc4)ccc32)c(-c2ccccc2)cc(-c2ccccc2)c1-n1c2ccc(-c3ccccc3)cc2n2c3cc(-c4ccccc4)ccc3nc12 ; Cc1c(N2C3=Nc4ccc(-c5ccccc5)cc4C3c3cc(-c4ccccc4)ccc32)c(-n2c3ccccc3c3ccccc32)cc(-n2c3ccccc3c3ccccc32)c1-n1c2ccc(-c3ccccc3)cc2n2c3cc(-c4ccccc4)ccc3nc12 ; Cc1c(N2C3=Nc4ccc(-c5ccccc5)cc4C3c3cc(-c4ccccc4)ccc32)c(-n2c3ccccc3c3ccccc32)cc(-c2ccccc2)c1-n1c2ccc(-c3ccccc3)cc2n2c3cc(-c4ccccc4)ccc3nc12]

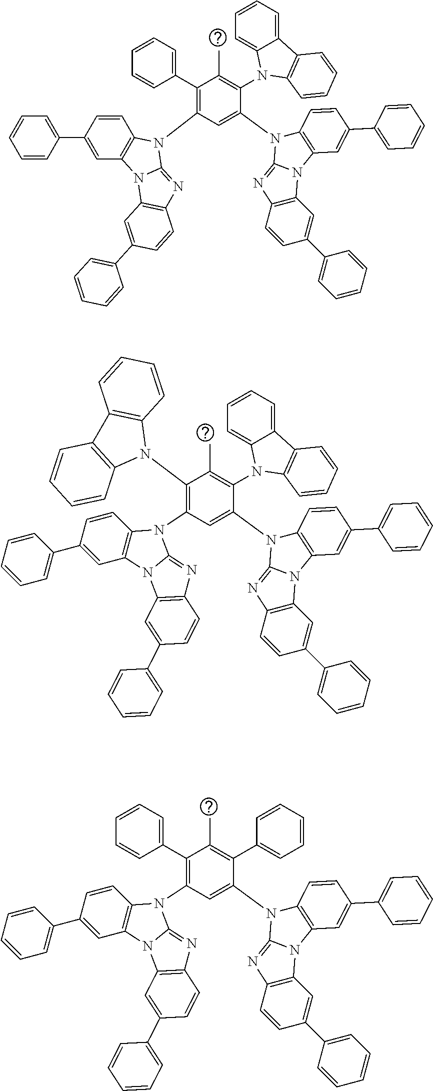 [SMILES: Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; Cc1c(-n2c3ccccc3c3ccccc32)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-c1ccccc1]

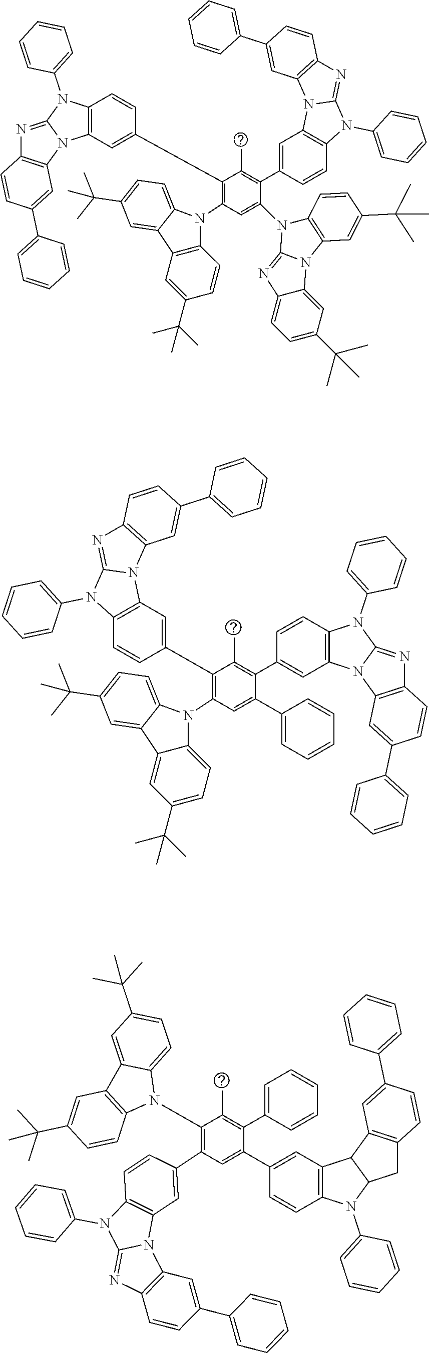

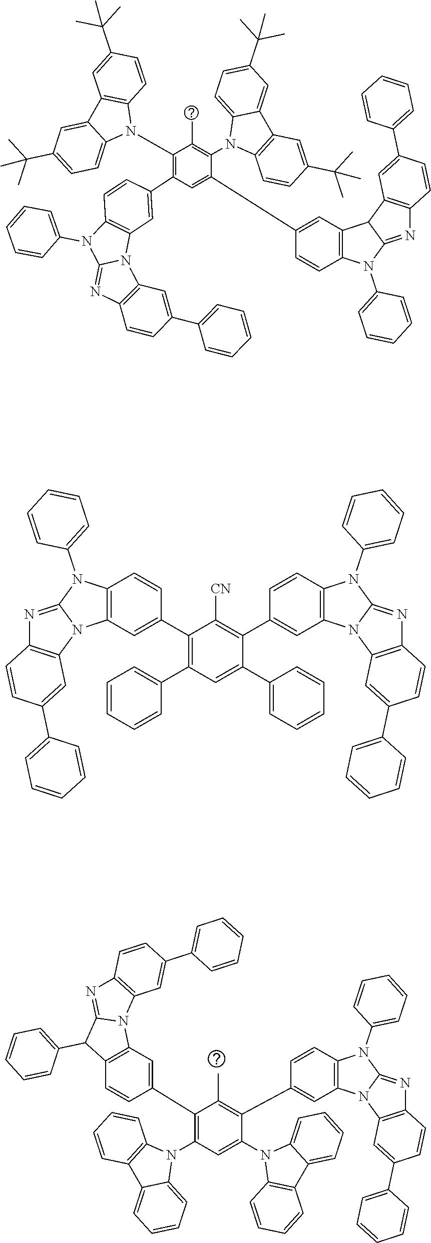 [SMILES: Cc1c(-n2c3ccc(C(C)(C)C)cc3c3cc(C(C)(C)C)ccc32)c(-c2ccc3c(c2)C2C(=Nc4ccc(-c5ccccc5)cc42)N3c2ccccc2)cc(-c2ccc3c(c2)n2c4cc(-c5ccccc5)ccc4nc2n3-c2ccccc2)c1-n1c2ccc(C(C)(C)C)cc2c2cc(C(C)(C)C)ccc21 ; N#Cc1c(-c2ccc3c(c2)n2c4cc(-c5ccccc5)ccc4nc2n3-c2ccccc2)c(-c2ccccc2)cc(-c2ccccc2)c1-c1ccc2c(c1)n1c3cc(-c4ccccc4)ccc3nc1n2-c1ccccc1 ; Cc1c(-c2ccc3c(c2)-n2c(nc4ccc(-c5ccccc5)cc42)C3c2ccccc2)c(-n2c3ccccc3c3ccccc32)cc(-n2c3ccccc3c3ccccc32)c1-c1ccc2c(c1)n1c3cc(-c4ccccc4)ccc3nc1n2-c1ccccc1]

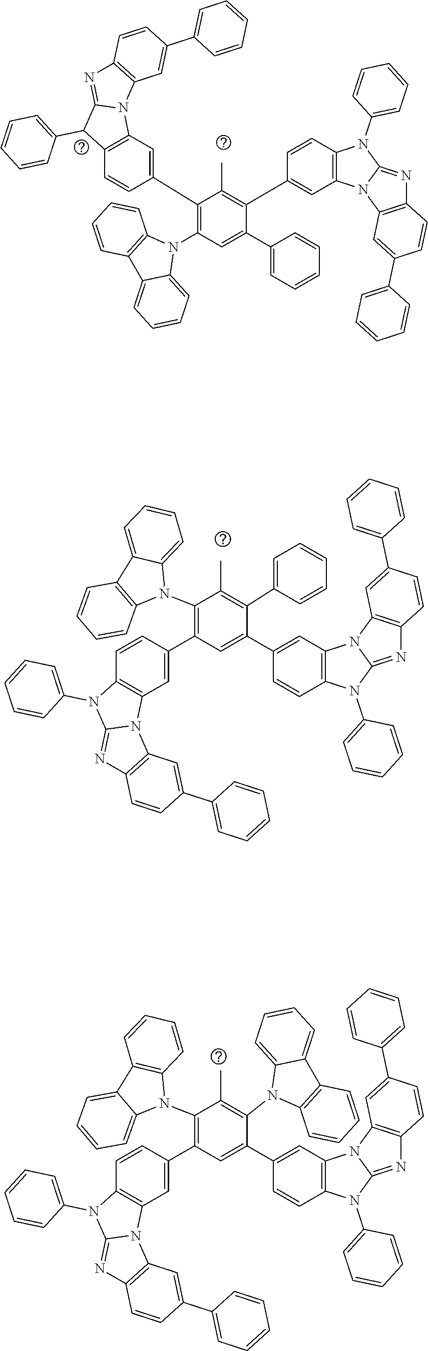 [SMILES: Cc1c(-c2ccc3c(c2)n2c4cc(-c5ccccc5)ccc4nc2n3-c2ccccc2)c(-c2ccccc2)cc(-n2c3ccccc3c3ccccc32)c1-c1ccc2c(c1)-n1c(nc3ccc(-c4ccccc4)cc31)C2c1ccccc1 ; Cc1c(-c2ccccc2)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-n1c2ccccc2c2ccccc21 ; Cc1c(-n2c3ccccc3c3ccccc32)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-n1c2ccccc2c2ccccc21]

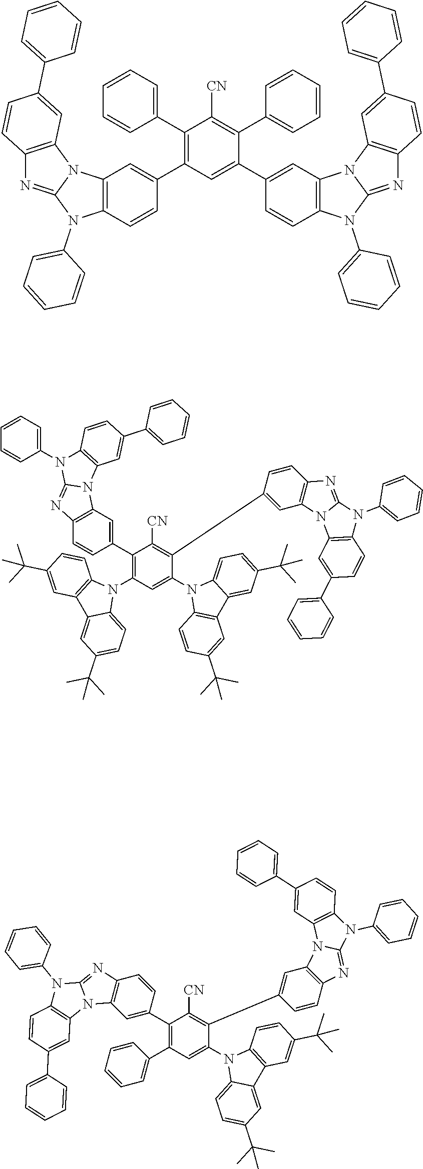 [SMILES: N#Cc1c(-c2ccccc2)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-c1ccccc1 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1 ; CC(C)(C)c1ccc2c(c1)c1cc(C(C)(C)C)ccc1n2-c1cc(-c2ccccc2)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1]

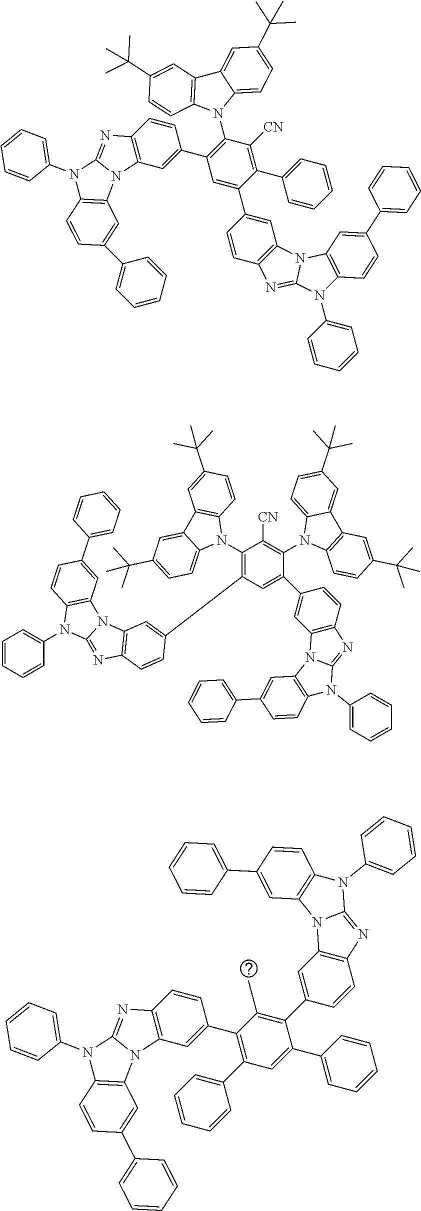 [SMILES: CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-c2ccccc2)c1C#N ; CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-n2c3ccc(C(C)(C)C)cc3c3cc(C(C)(C)C)ccc32)c1C#N ; Cc1c(-c2ccc3nc4n(-c5ccccc5)c5ccc(-c6ccccc6)cc5n4c3c2)c(-c2ccccc2)cc(-c2ccccc2)c1-c1ccc2nc3n(-c4ccccc4)c4ccc(-c5ccccc5)cc4n3c2c1]

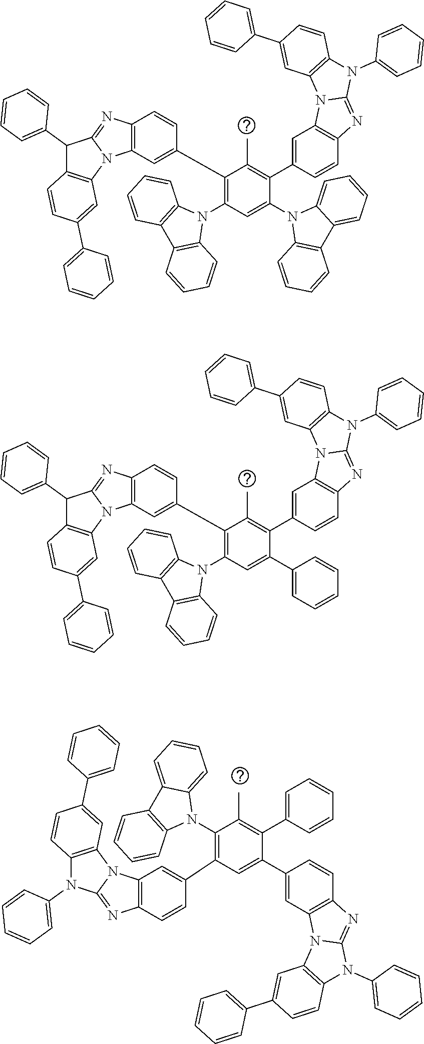 [SMILES: Cc1c(-c2ccc3nc4n(c3c2)-c2cc(-c3ccccc3)ccc2C4c2ccccc2)c(-n2c3ccccc3c3ccccc32)cc(-n2c3ccccc3c3ccccc32)c1-c1ccc2nc3n(-c4ccccc4)c4ccc(-c5ccccc5)cc4n3c2c1 ; Cc1c(-c2ccc3nc4n(-c5ccccc5)c5ccc(-c6ccccc6)cc5n4c3c2)c(-c2ccccc2)cc(-n2c3ccccc3c3ccccc32)c1-c1ccc2nc3n(c2c1)-c1cc(-c2ccccc2)ccc1C3c1ccccc1 ; Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21]

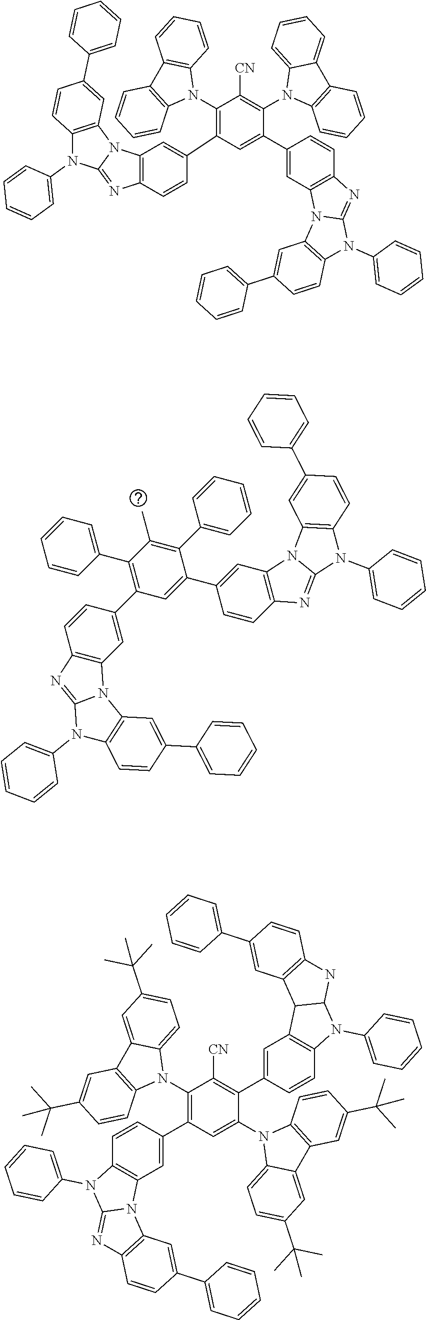 [SMILES: N#Cc1c(-n2c3ccccc3c3ccccc32)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-c1ccccc1 ; CC(C)(C)c1ccc2c(c1)c1cc(C(C)(C)C)ccc1n2-c1cc(-c2ccc3c(c2)n2c4cc(-c5ccccc5)ccc4nc2n3-c2ccccc2)c(-n2c3ccc(C(C)(C)C)cc3c3cc(C(C)(C)C)ccc32)c(C#N)c1-c1ccc2c(c1)C1c3cc(-c4ccccc4)ccc3NC1N2c1ccccc1]

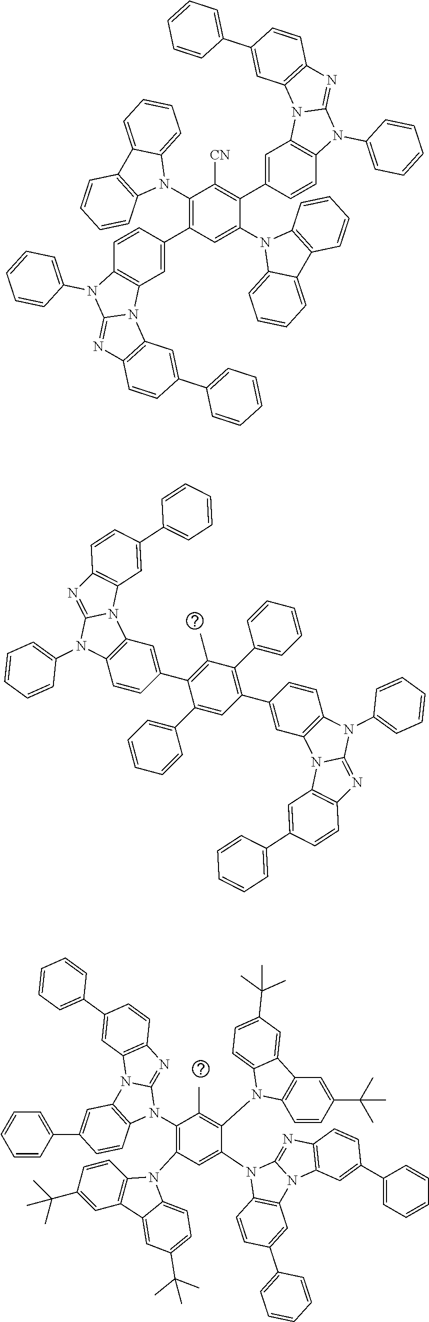 [SMILES: N#Cc1c(-c2ccc3c(c2)n2c4cc(-c5ccccc5)ccc4nc2n3-c2ccccc2)c(-n2c3ccccc3c3ccccc32)cc(-c2ccc3c(c2)n2c4cc(-c5ccccc5)ccc4nc2n3-c2ccccc2)c1-n1c2ccccc2c2ccccc21 ; Cc1c(-c2ccccc2)c(-c2ccc3c(c2)n2c4cc(-c5ccccc5)ccc4nc2n3-c2ccccc2)cc(-c2ccccc2)c1-c1ccc2c(c1)n1c3cc(-c4ccccc4)ccc3nc1n2-c1ccccc1 ; Cc1c(-n2c3ccc(C(C)(C)C)cc3c3cc(C(C)(C)C)ccc32)c(-n2c3ccc(-c4ccccc4)cc3n3c4cc(-c5ccccc5)ccc4nc23)cc(-n2c3ccc(C(C)(C)C)cc3c3cc(C(C)(C)C)ccc32)c1-n1c2ccc(-c3ccccc3)cc2n2c3cc(-c4ccccc4)ccc3nc12]

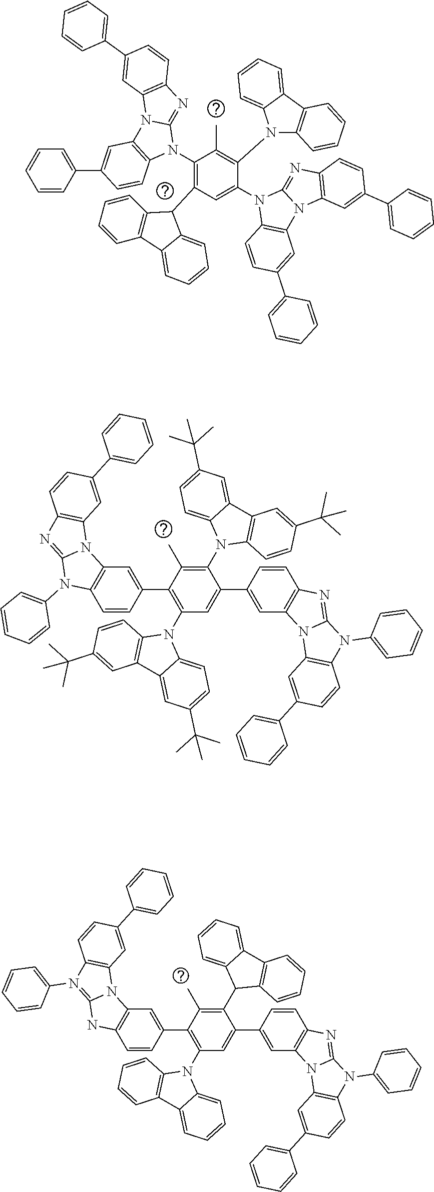

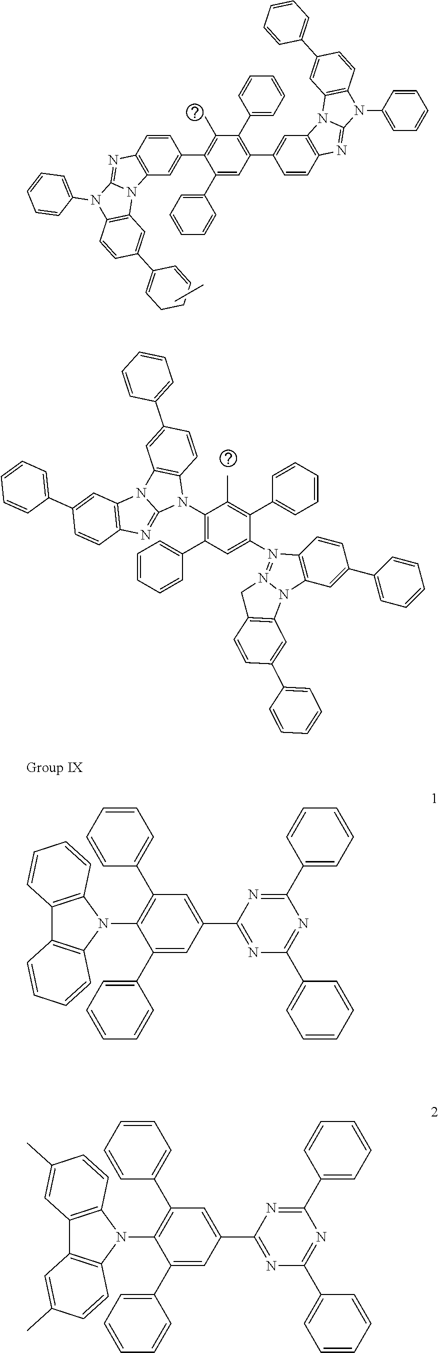

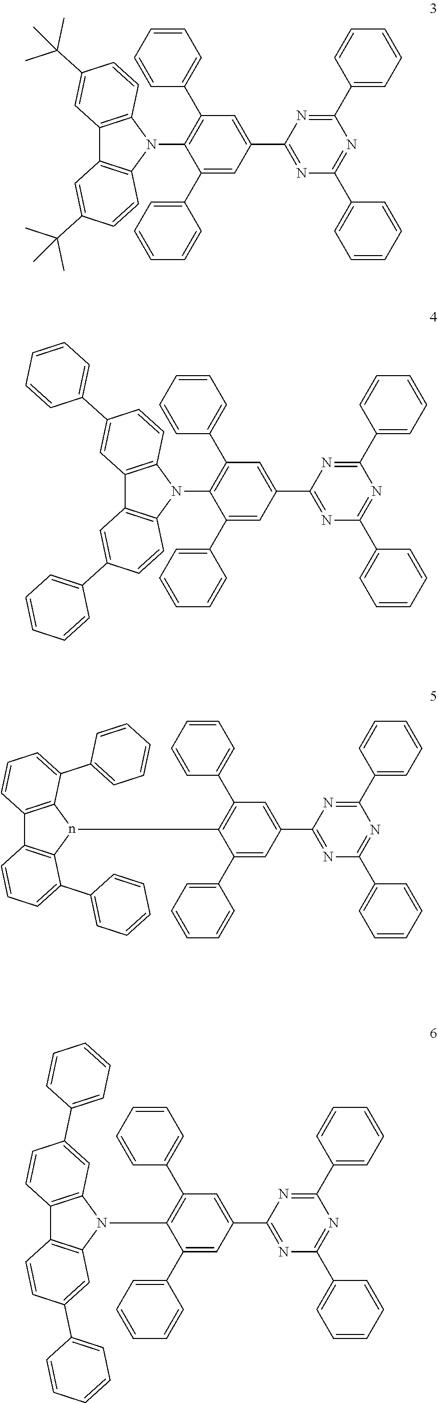

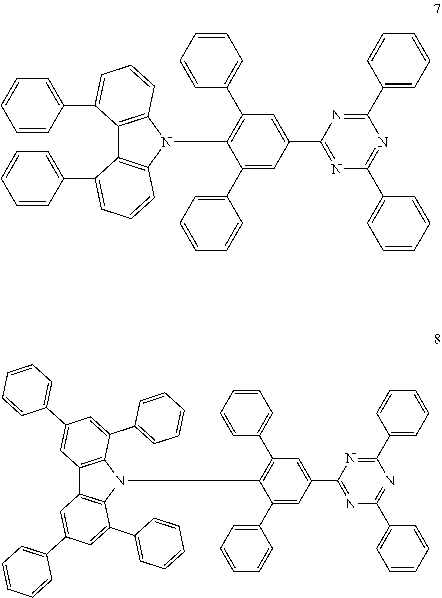 [SMILES: c1ccc(-c2nc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1]

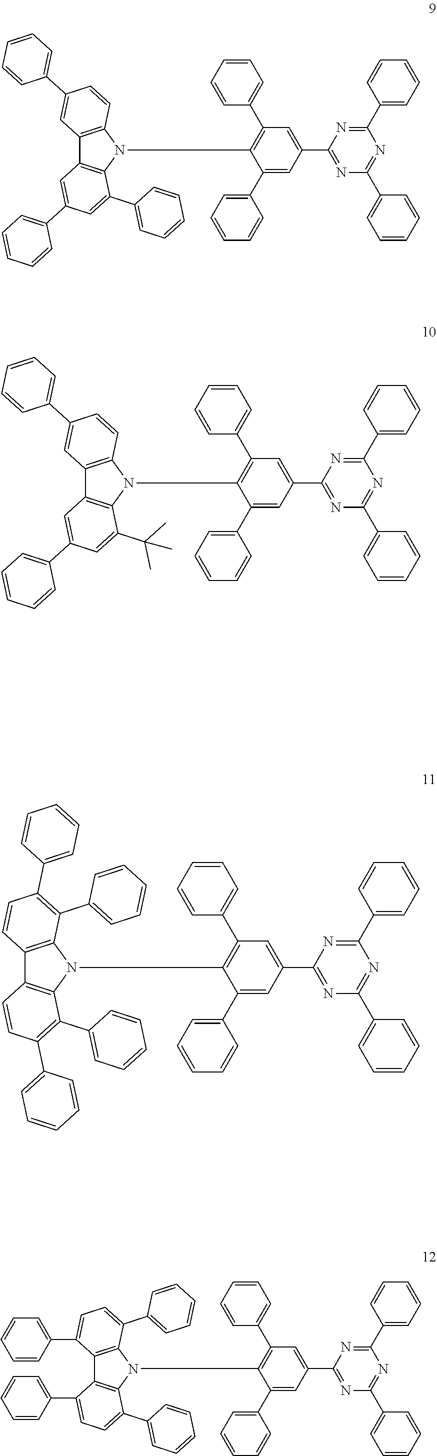 [SMILES: c1ccc(-c2ccc3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)nc(-c5ccccc5)n4)cc3-c3ccccc3)c12 ; c1ccc(-c2nc(-c3ccccc3)nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)n2)cc1 ; c1ccc(-c2nc(-c3ccccc3)nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)n2)cc1]

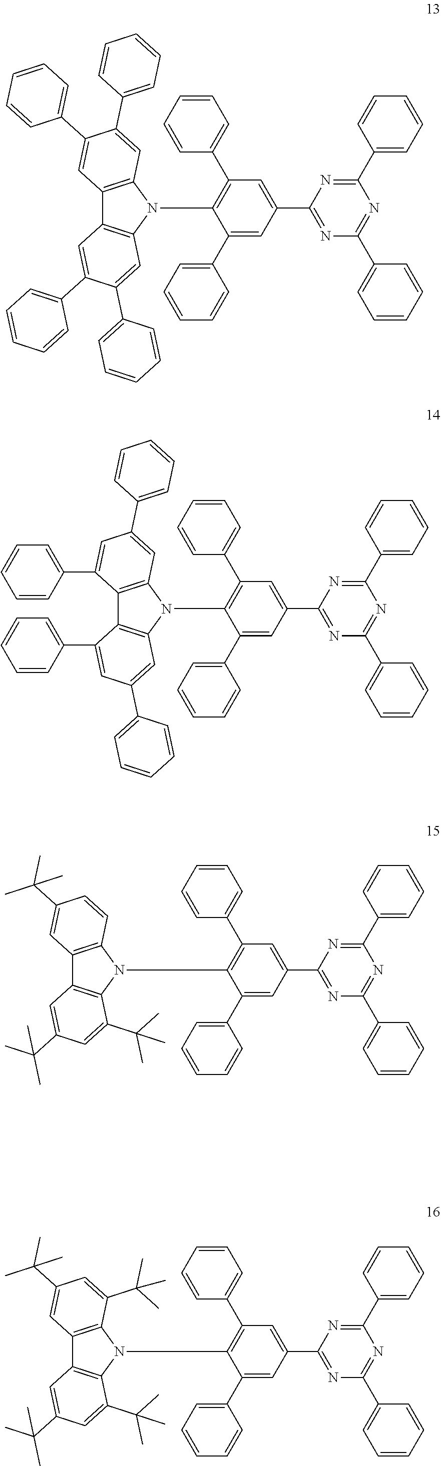 [SMILES: c1ccc(-c2nc(-c3ccccc3)nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)nc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1]

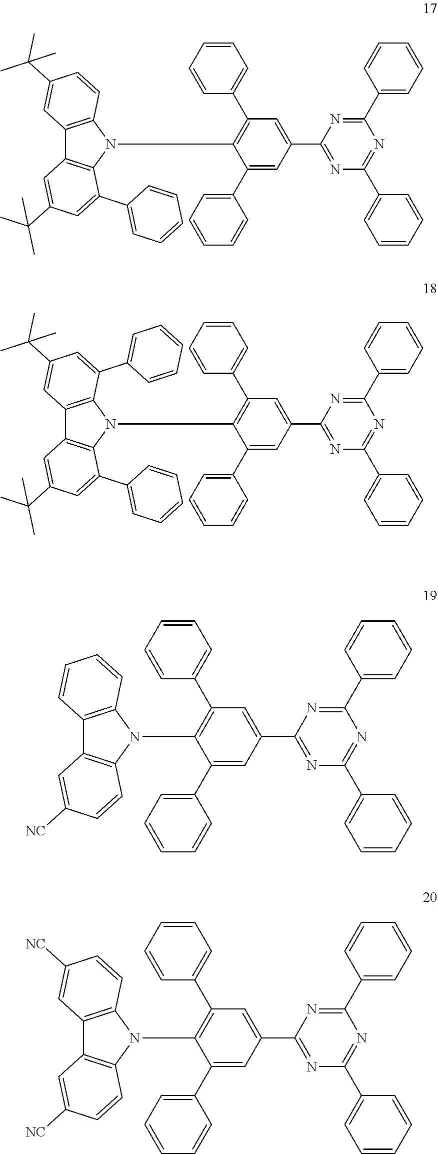

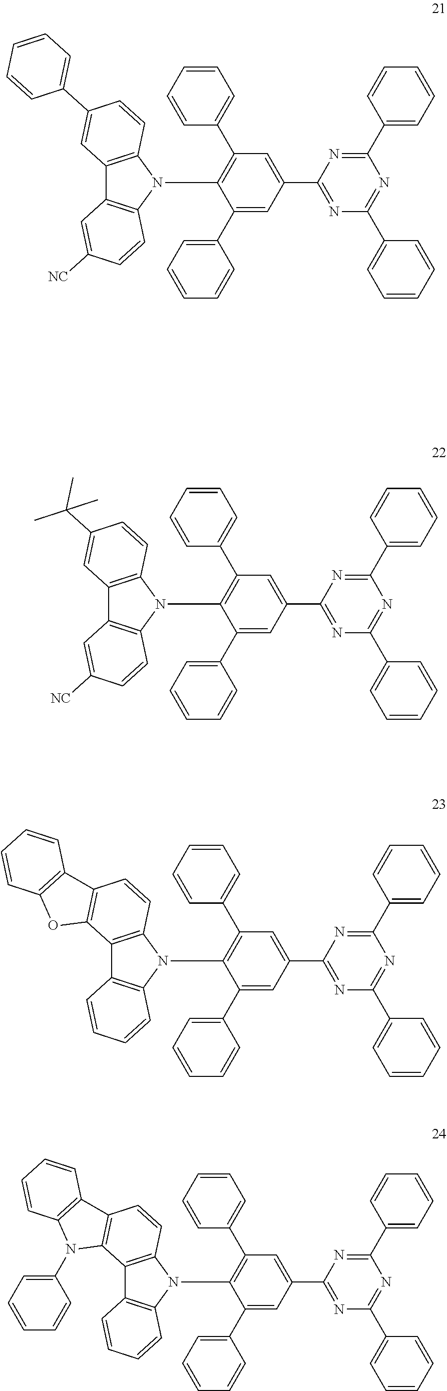

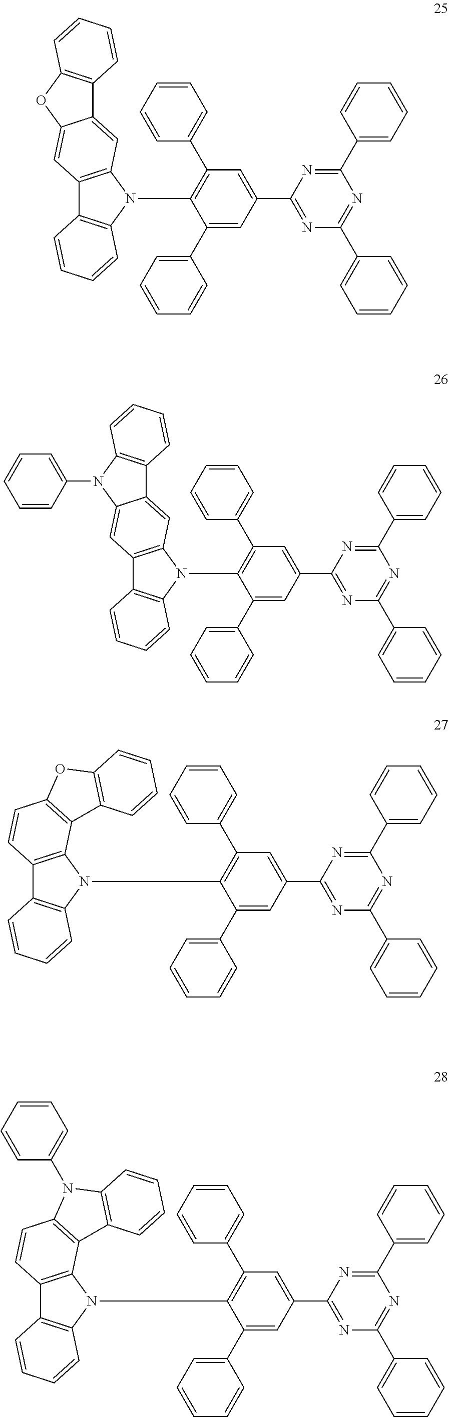 [SMILES: c1ccc(-c2nc(-c3ccccc3)nc(-c3cc(-c4ccccc4)c(-n4c5ccccc5c5cc6oc7ccccc7c6cc54)c(-c4ccccc4)c3)n2)cc1 ; c1ccc(-c2nc(-c3ccccc3)nc(-c3cc(-c4ccccc4)c(-n4c5ccccc5c5cc6c(cc54)c4ccccc4n6-c4ccccc4)c(-c4ccccc4)c3)n2)cc1 ; c1ccc(-c2nc(-c3ccccc3)nc(-c3cc(-c4ccccc4)c(-n4c5ccccc5c5ccc6oc7ccccc7c6c54)c(-c4ccccc4)c3)n2)cc1 ; c1ccc(-c2nc(-c3ccccc3)nc(-c3cc(-c4ccccc4)c(-n4c5ccccc5c5ccc6c(c7ccccc7n6-c6ccccc6)c54)c(-c4ccccc4)c3)n2)cc1]

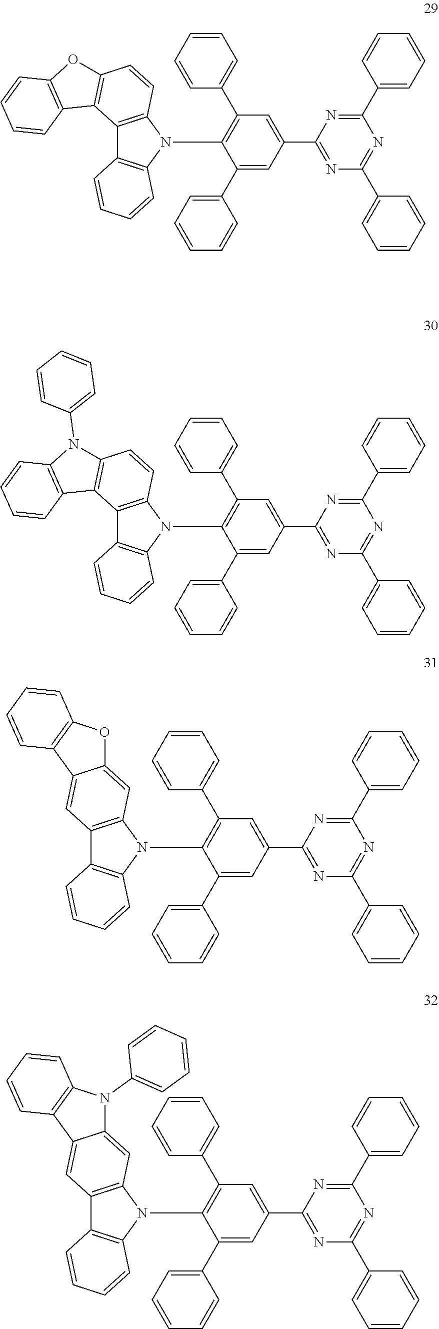 [SMILES: c1ccc(-c2nc(-c3ccccc3)nc(-c3cc(-c4ccccc4)c(-n4c5ccccc5c5c6c(ccc54)oc4ccccc46)c(-c4ccccc4)c3)n2)cc1 ; c1ccc(-c2nc(-c3ccccc3)nc(-c3cc(-c4ccccc4)c(-n4c5ccccc5c5c6c7ccccc7n(-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2nc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1]

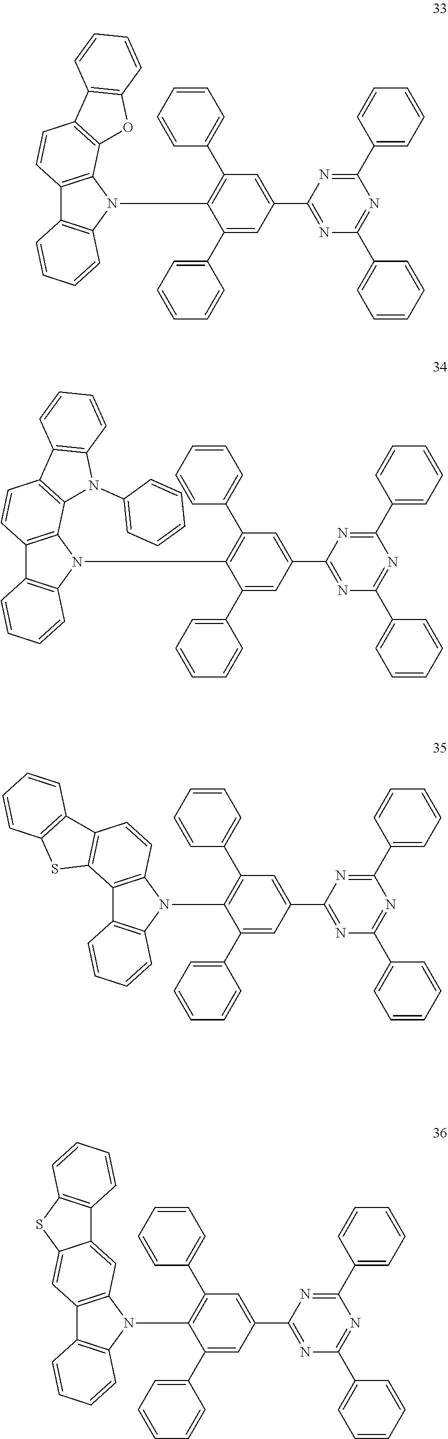 [SMILES: c1ccc(-c2nc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2nc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1 ; c1ccc(-c2nc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2nc(-c3ccccc3)nc(-c3cc(-c4ccccc4)c(-n4c5ccccc5c5cc6sc7ccccc7c6cc54)c(-c4ccccc4)c3)n2)cc1]

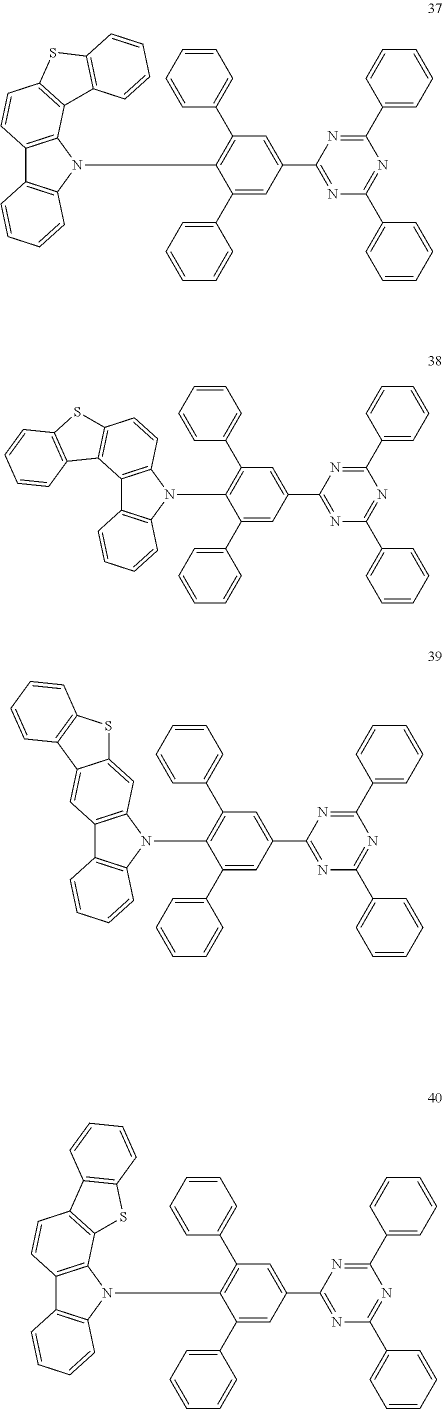 [SMILES: c1ccc(-c2nc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2nc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2nc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2nc(-c3ccccc3)nc(-c3cc(-c4ccccc4)c(-n4c5ccccc5c5ccc6c7ccccc7sc6c54)c(-c4ccccc4)c3)n2)cc1]

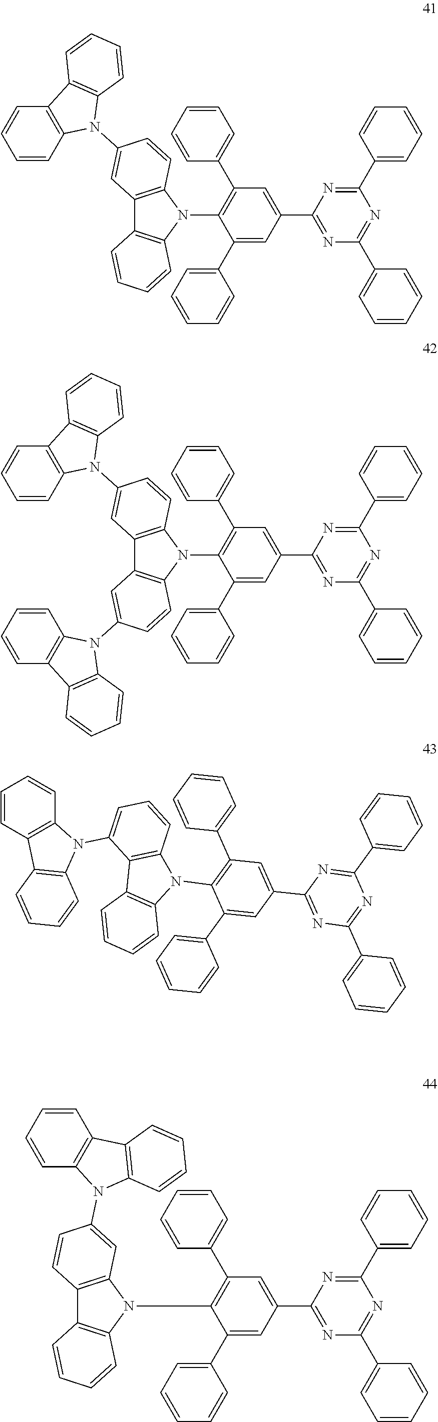 [SMILES: c1ccc(-c2nc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2nc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

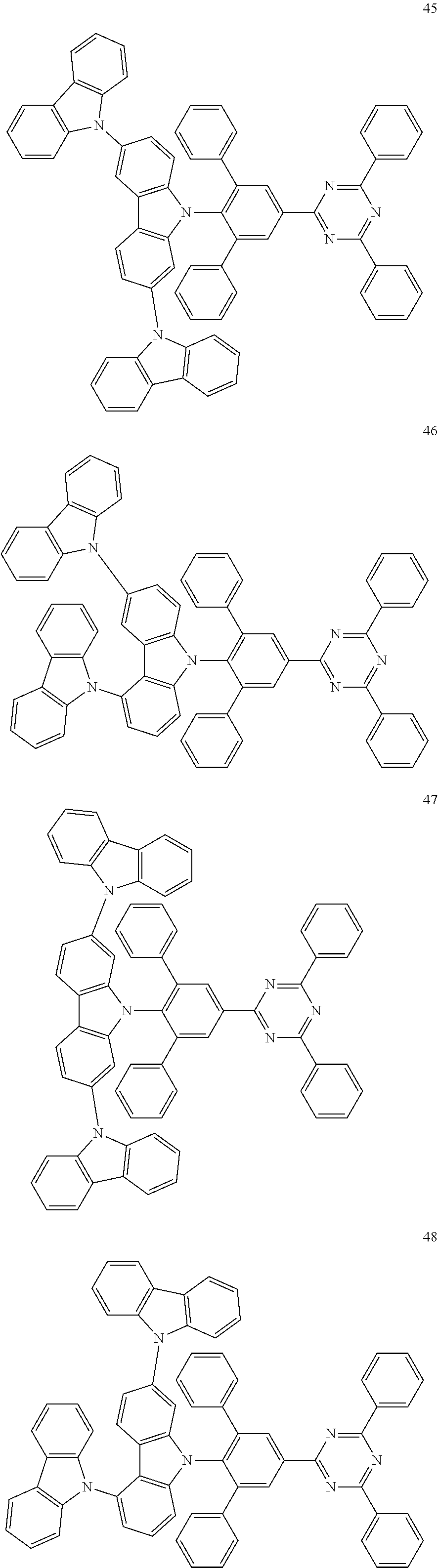

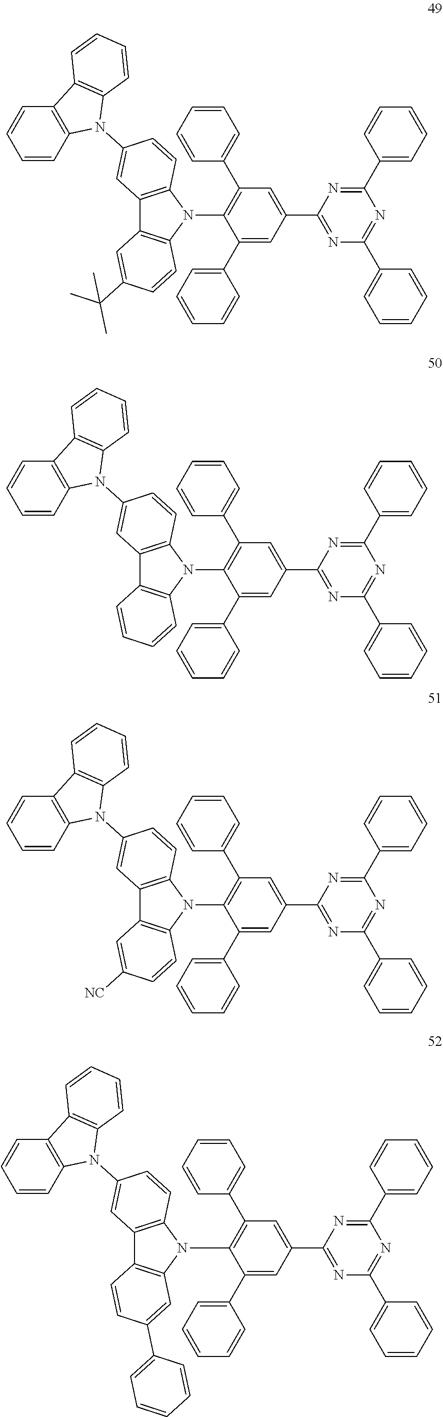 [SMILES: CC(C)(C)c1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1 ; c1ccc(-c2nc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; N#Cc1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5nc(-c6ccccc6)nc(-c6ccccc6)n5)cc4-c4ccccc4)c3c2)cc1]

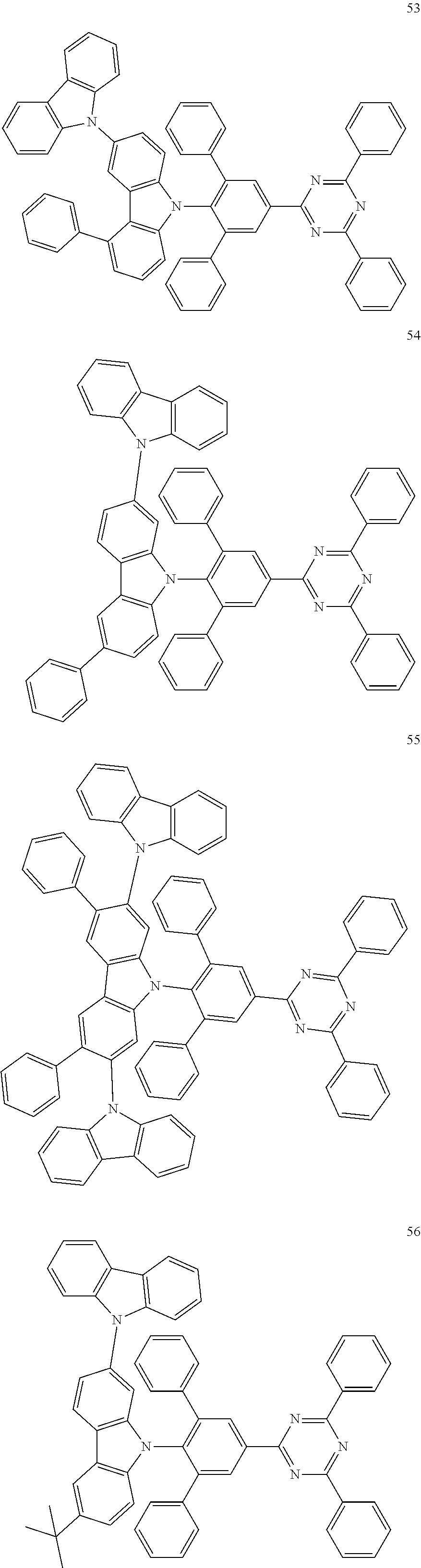

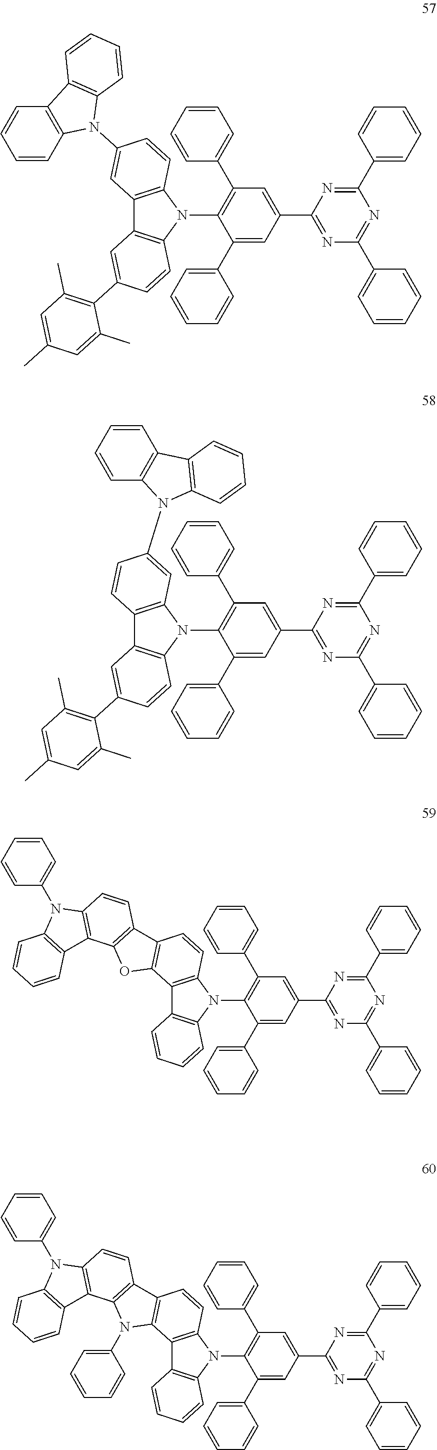

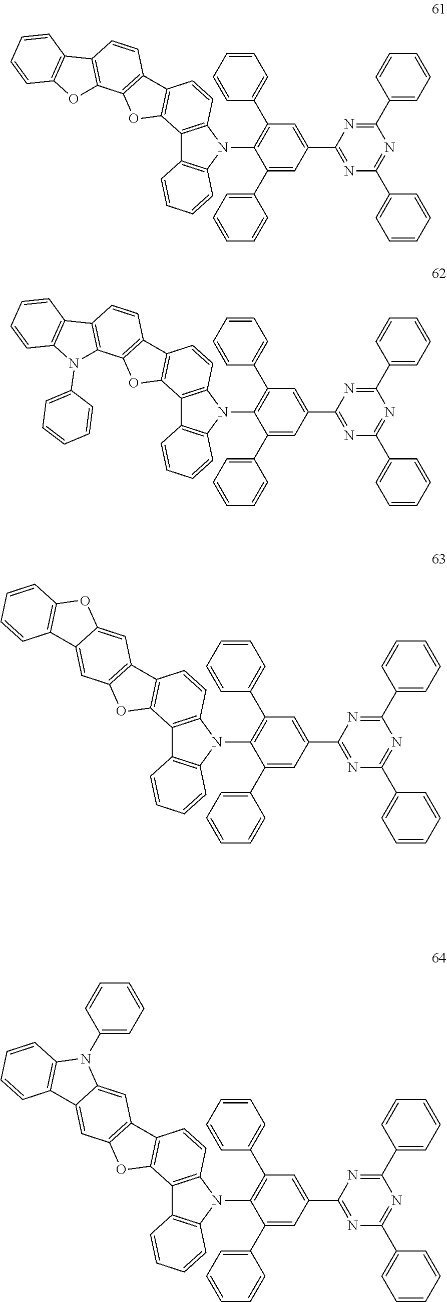 [SMILES: c1ccc(-c2nc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2nc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1]

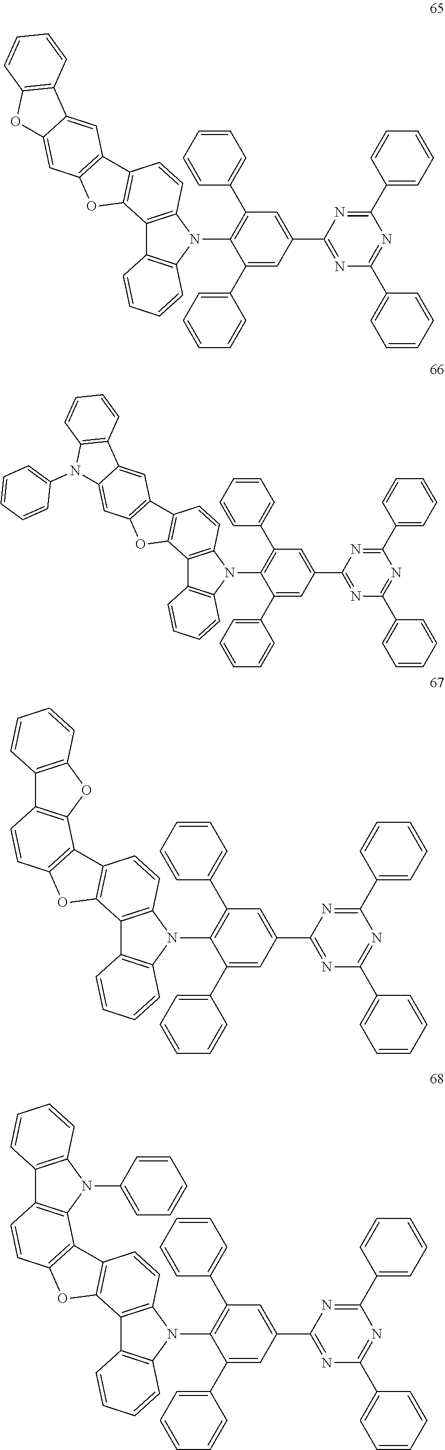 [SMILES: c1ccc(-c2nc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1]

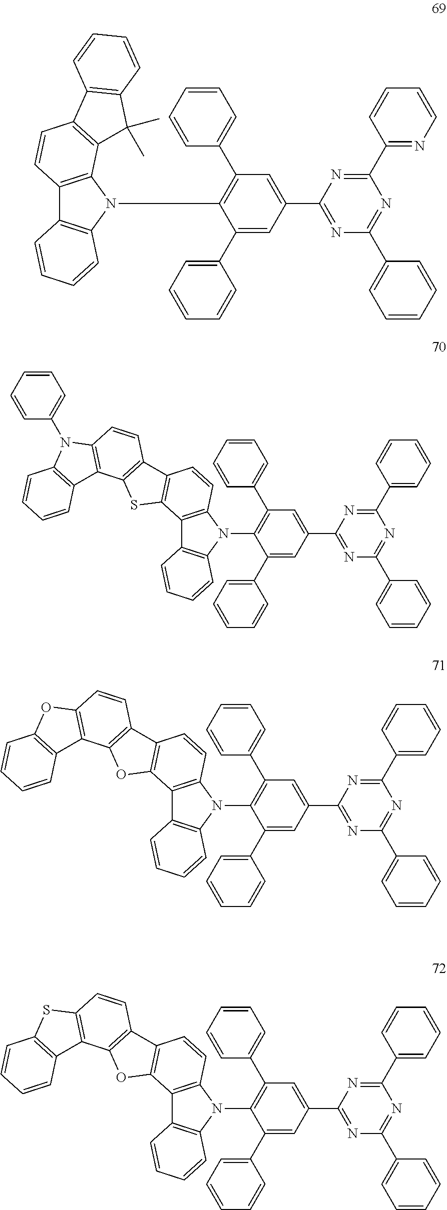 [SMILES: CC1(C)c2ccccc2-c2ccc3c4ccccc4n(-c4c(-c5ccccc5)cc(-c5nc(-c6ccccc6)nc(-c6ccccn6)n5)cc4-c4ccccc4)c3c21 ; c1ccc(-c2nc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1]

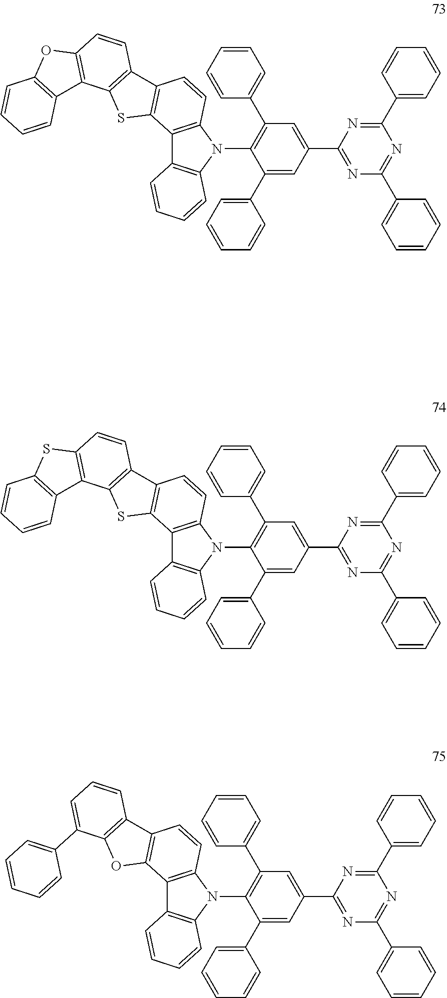 [SMILES: c1ccc(-c2nc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1]

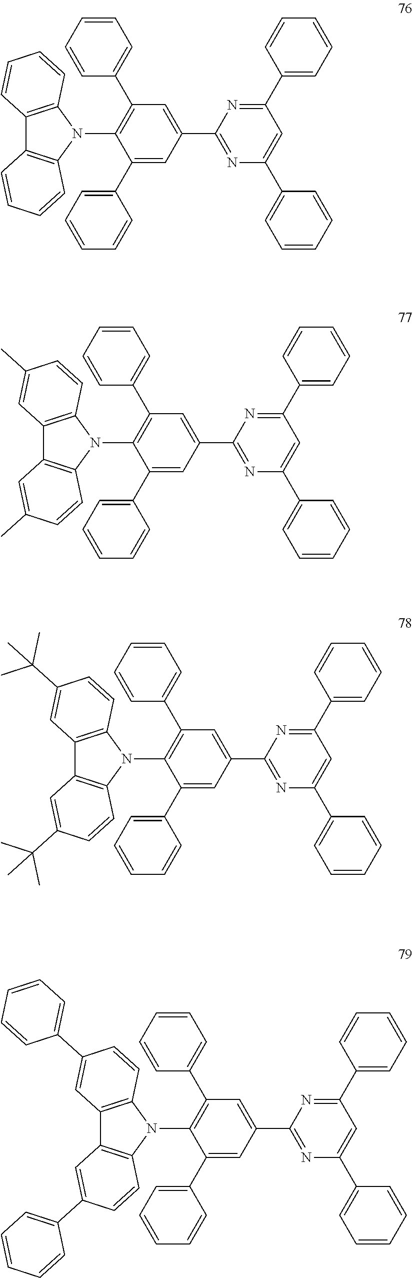 [SMILES: c1ccc(-c2cc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)cc(-c3ccccc3)n2)cc1-c1ccccc1 ; CC(C)(C)c1ccc2c(c1)c1cc(C(C)(C)C)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)cc(-c4ccccc4)n3)cc2-c2ccccc2)cc1]

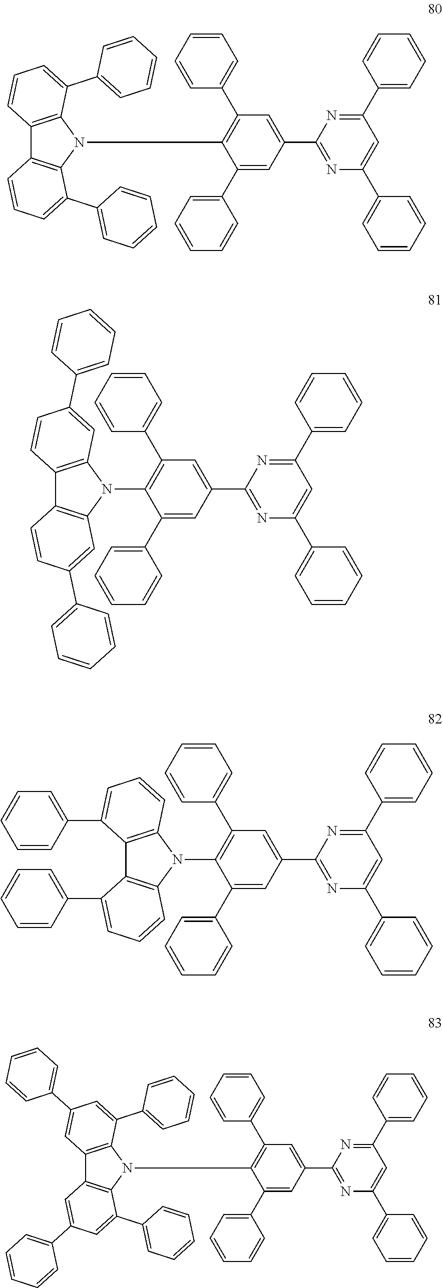 [SMILES: c1ccc(-c2cc(-c3ccccc3)nc(-c3cc(-c4ccccc4)c(-n4c5c(-c6ccccc6)cccc5c5cccc(-c6ccccc6)c54)c(-c4ccccc4)c3)n2)cc1 ; c1ccc(-c2ccc3c4ccc(-c5ccccc5)cc4n(-c4c(-c5ccccc5)cc(-c5nc(-c6ccccc6)cc(-c6ccccc6)n5)cc4-c4ccccc4)c3c2)cc1 ; c1ccc(-c2cc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1]

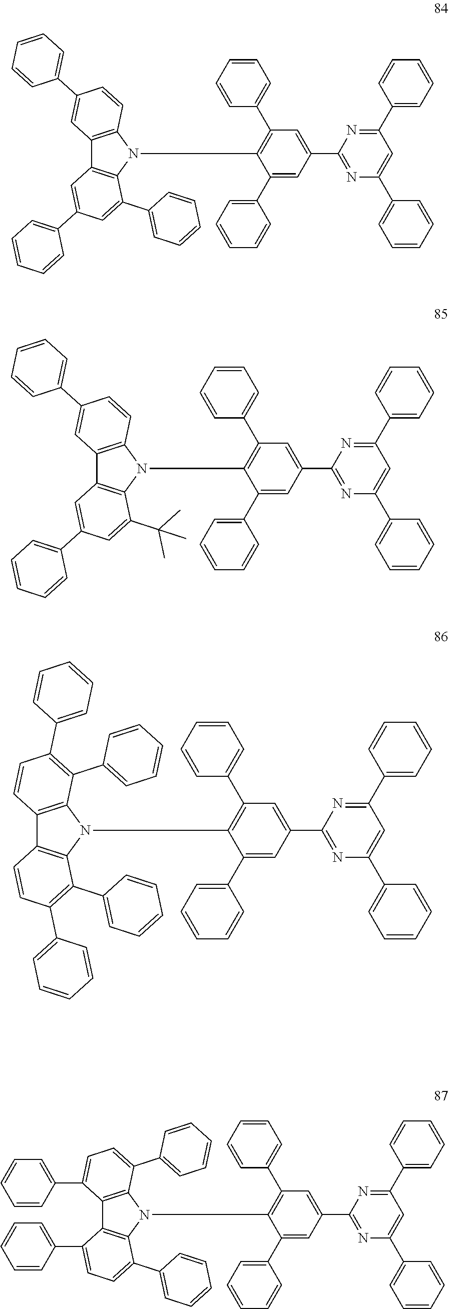 [SMILES: c1ccc(-c2ccc3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)cc(-c5ccccc5)n4)cc3-c3ccccc3)c12 ; c1ccc(-c2cc(-c3ccccc3)nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)n2)cc1 ; c1ccc(-c2cc(-c3ccccc3)nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)n2)cc1]

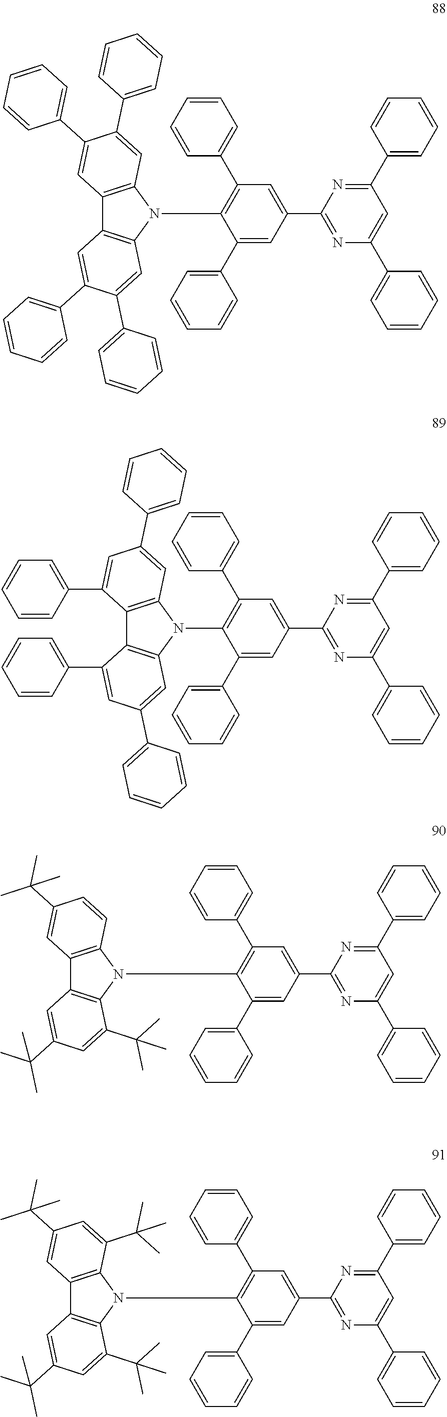 [SMILES: c1ccc(-c2cc(-c3ccccc3)nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)cc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1]

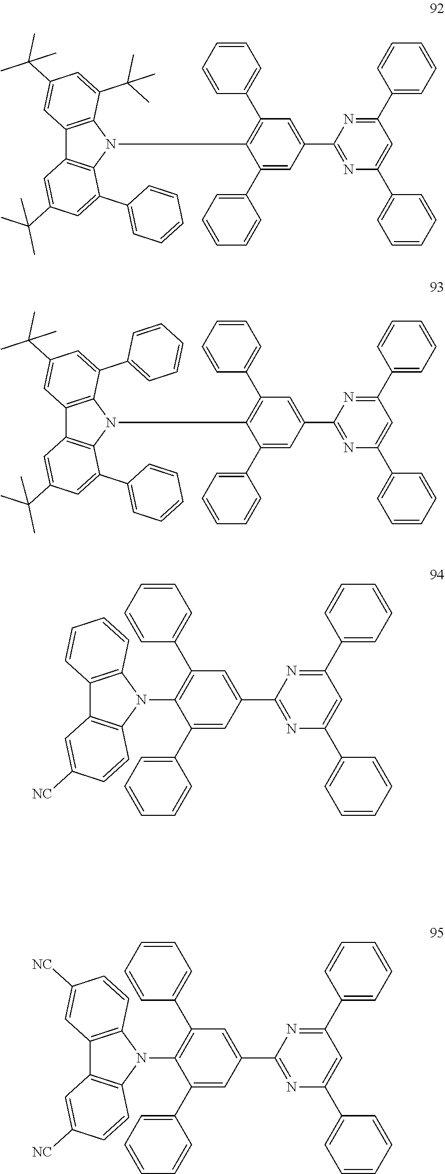 [SMILES: CC(C)(C)c1cc(-c2ccccc2)c2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)cc(-c3ccccc3)n2)cc1-c1ccccc1 ; N#Cc1ccc2c(c1)c1ccccc1n2-c1c(-c2ccccc2)cc(-c2nc(-c3ccccc3)cc(-c3ccccc3)n2)cc1-c1ccccc1 ; N#Cc1ccc2c(c1)c1cc(C#N)ccc1n2-c1c(-c2ccccc2)cc(-c2nc(-c3ccccc3)cc(-c3ccccc3)n2)cc1-c1ccccc1]

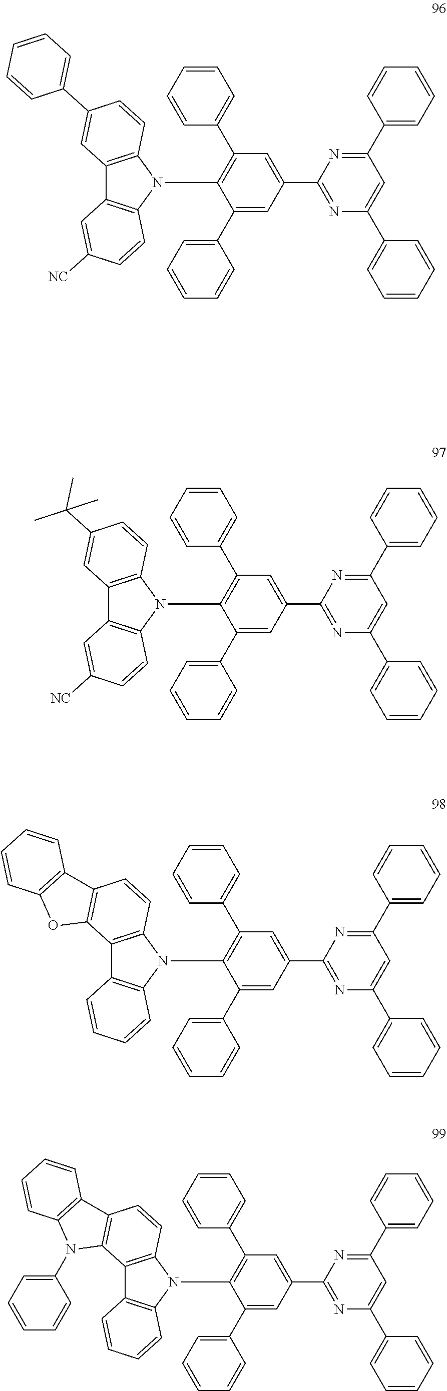

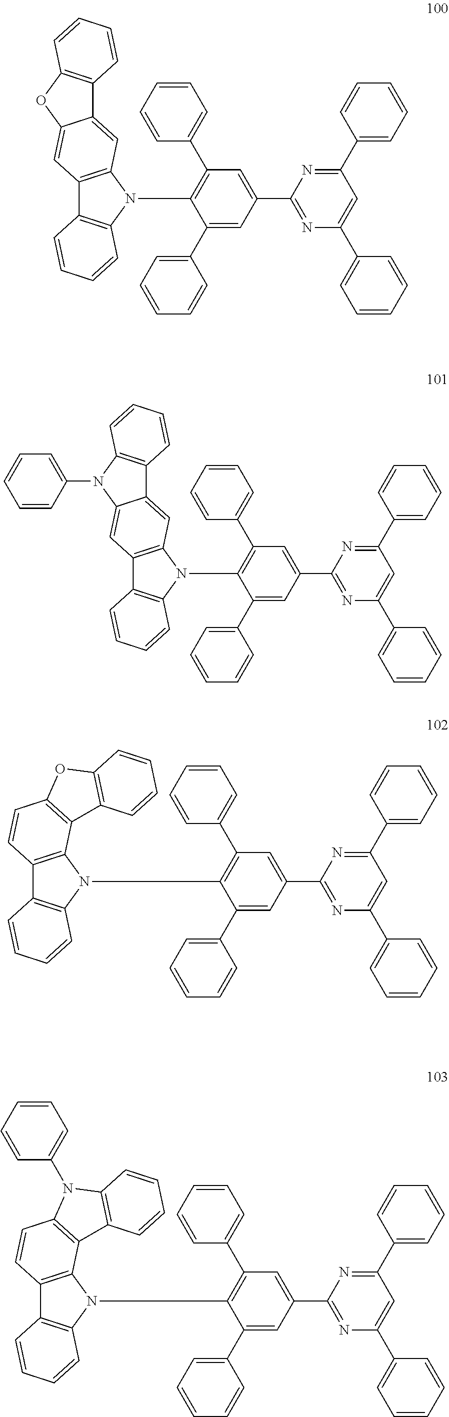 [SMILES: c1ccc(-c2cc(-c3ccccc3)nc(-c3cc(-c4ccccc4)c(-n4c5ccccc5c5cc6oc7ccccc7c6cc54)c(-c4ccccc4)c3)n2)cc1 ; c1ccc(-c2cc(-c3ccccc3)nc(-c3cc(-c4ccccc4)c(-n4c5ccccc5c5cc6c(cc54)c4ccccc4n6-c4ccccc4)c(-c4ccccc4)c3)n2)cc1 ; c1ccc(-c2cc(-c3ccccc3)nc(-c3cc(-c4ccccc4)c(-n4c5ccccc5c5ccc6oc7ccccc7c6c54)c(-c4ccccc4)c3)n2)cc1 ; c1ccc(-c2cc(-c3ccccc3)nc(-c3cc(-c4ccccc4)c(-n4c5ccccc5c5ccc6c(c7ccccc7n6-c6ccccc6)c54)c(-c4ccccc4)c3)n2)cc1]

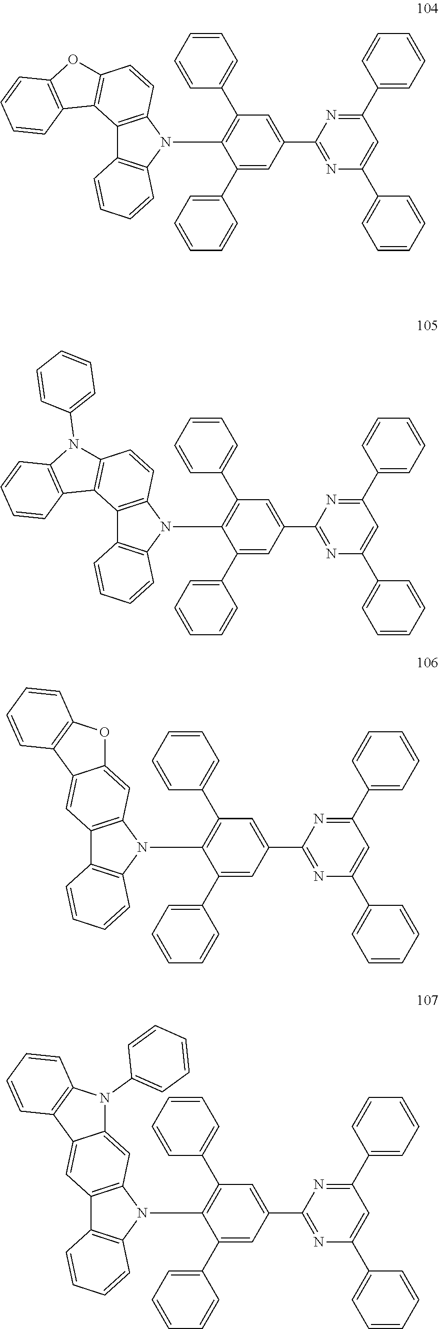 [SMILES: c1ccc(-c2cc(-c3ccccc3)nc(-c3cc(-c4ccccc4)c(-n4c5ccccc5c5c6c(ccc54)oc4ccccc46)c(-c4ccccc4)c3)n2)cc1 ; c1ccc(-c2cc(-c3ccccc3)nc(-c3cc(-c4ccccc4)c(-n4c5ccccc5c5c6c7ccccc7n(-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2cc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1]

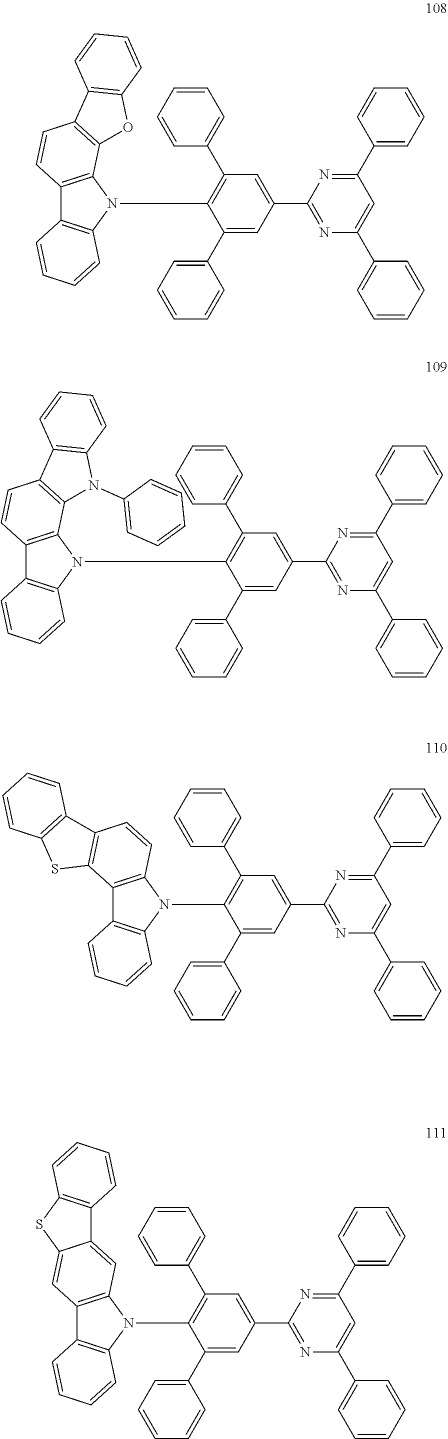 [SMILES: c1ccc(-c2cc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2cc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1 ; c1ccc(-c2cc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2cc(-c3ccccc3)nc(-c3cc(-c4ccccc4)c(-n4c5ccccc5c5cc6sc7ccccc7c6cc54)c(-c4ccccc4)c3)n2)cc1]

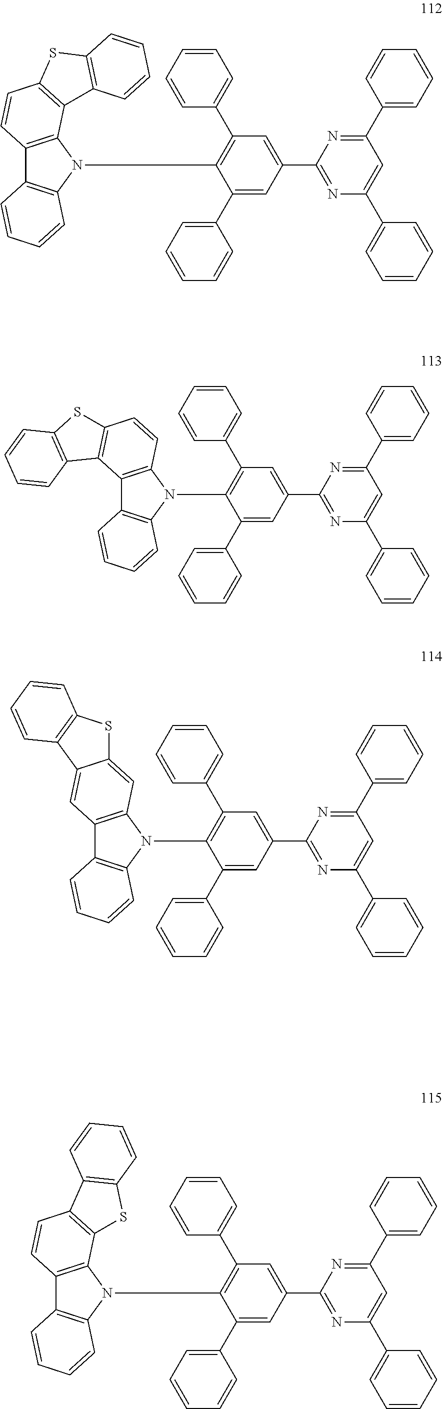 [SMILES: c1ccc(-c2cc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2cc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2cc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2cc(-c3ccccc3)nc(-c3cc(-c4ccccc4)c(-n4c5ccccc5c5ccc6c7ccccc7sc6c54)c(-c4ccccc4)c3)n2)cc1]

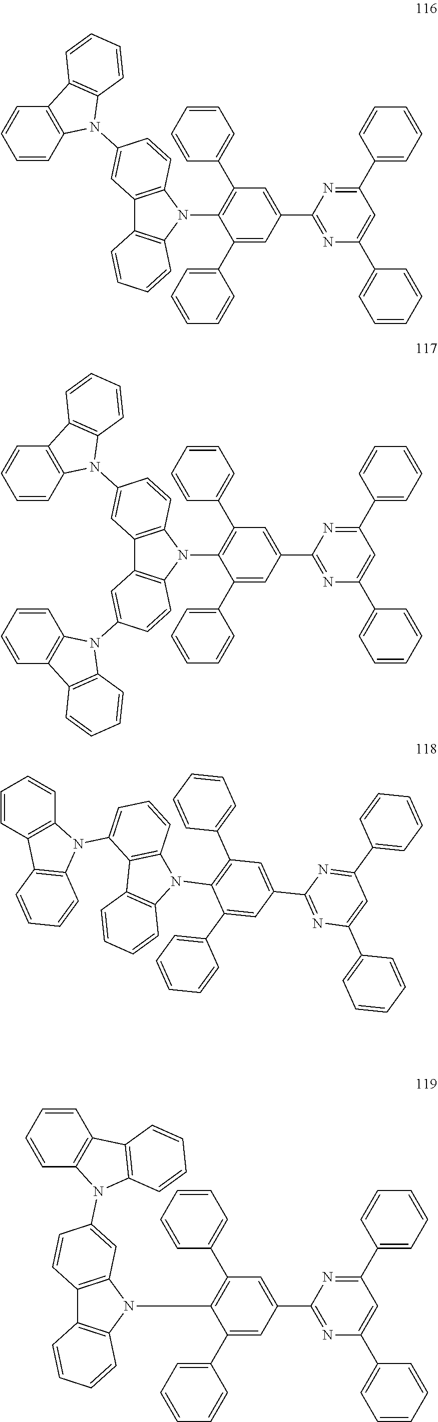 [SMILES: c1ccc(-c2cc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2cc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

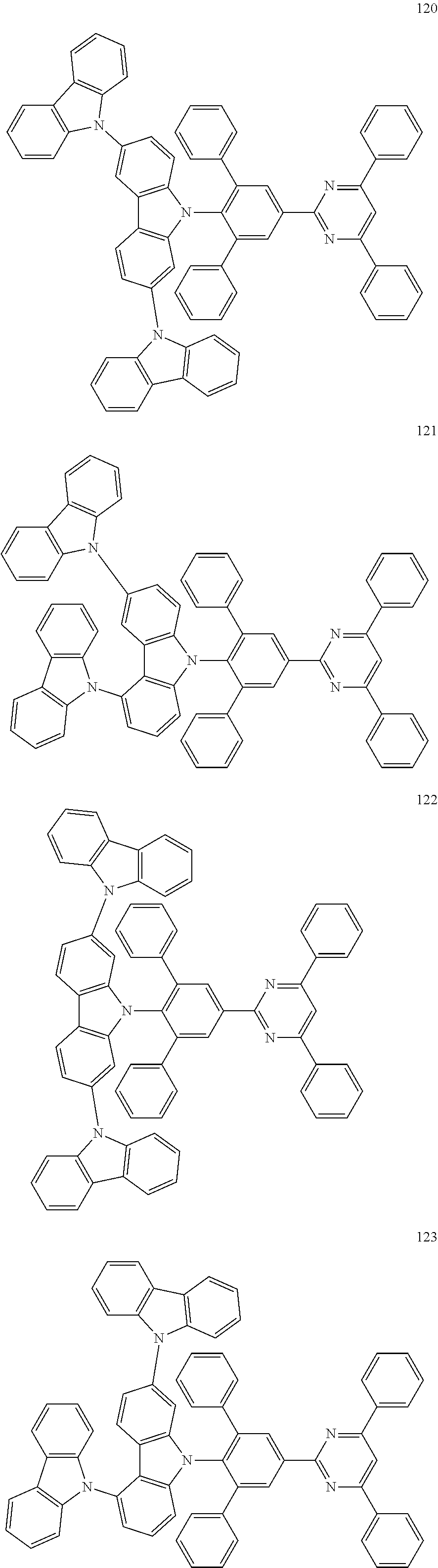

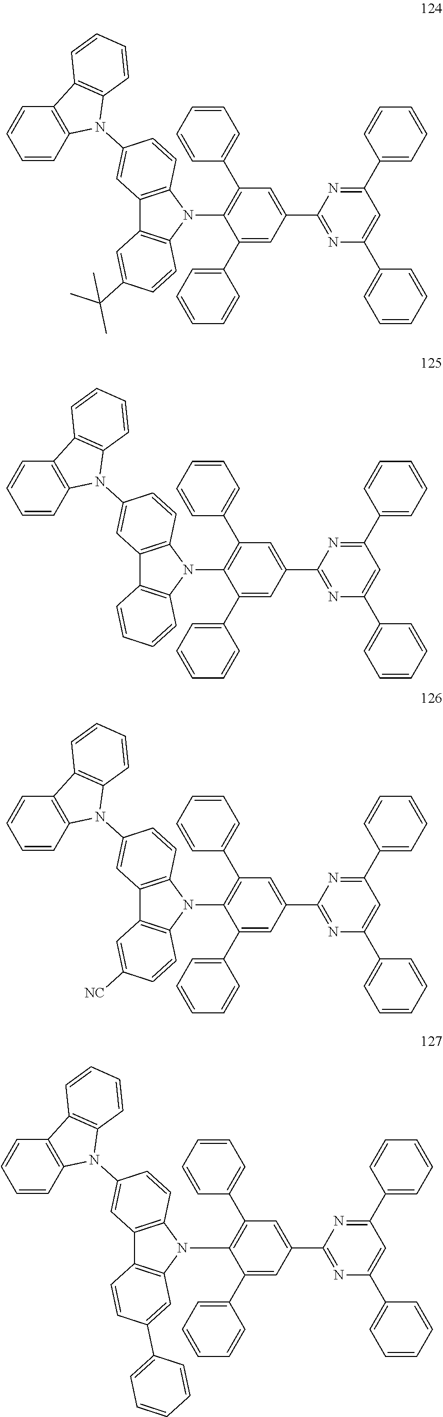 [SMILES: CC(C)(C)c1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1 ; c1ccc(-c2cc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; N#Cc1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5nc(-c6ccccc6)cc(-c6ccccc6)n5)cc4-c4ccccc4)c3c2)cc1]

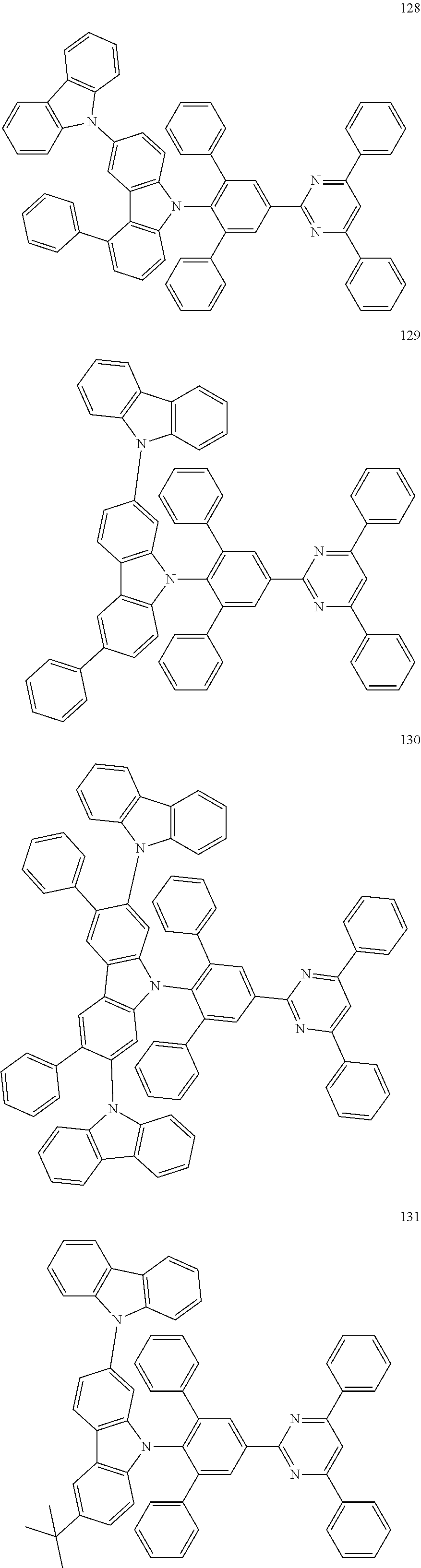

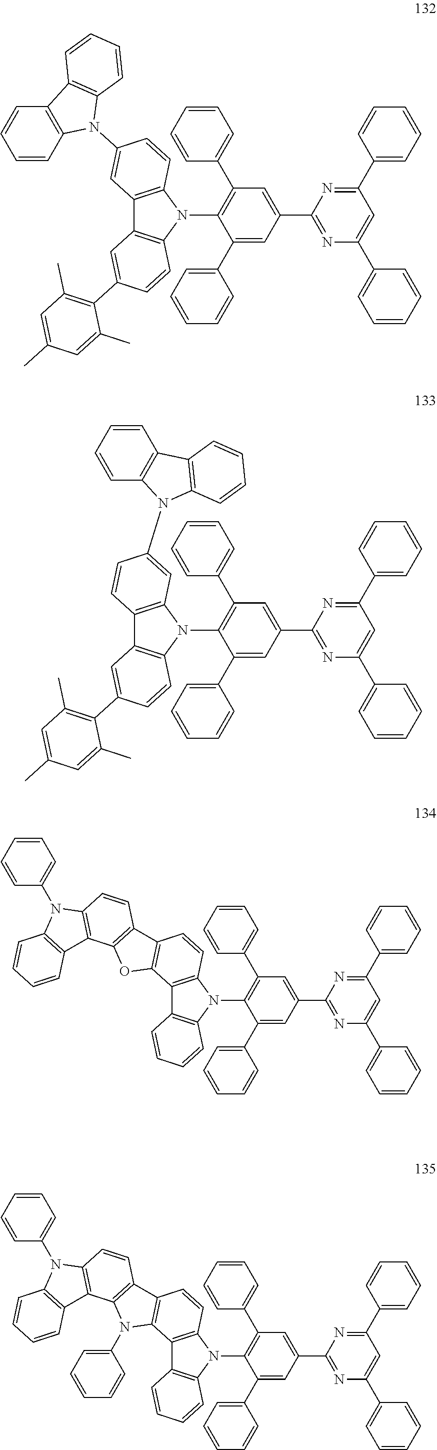

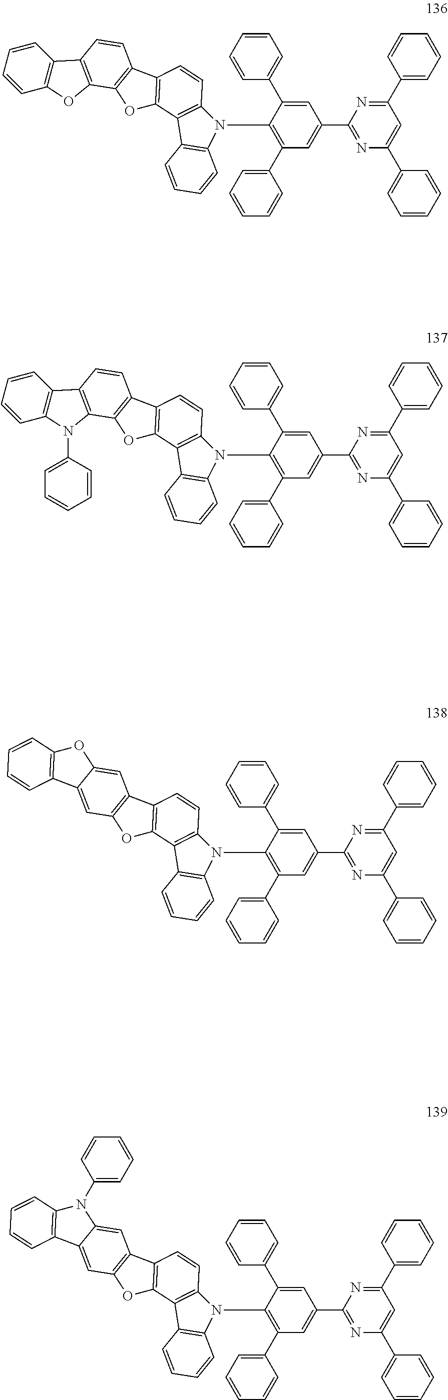 [SMILES: c1ccc(-c2cc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2cc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1]

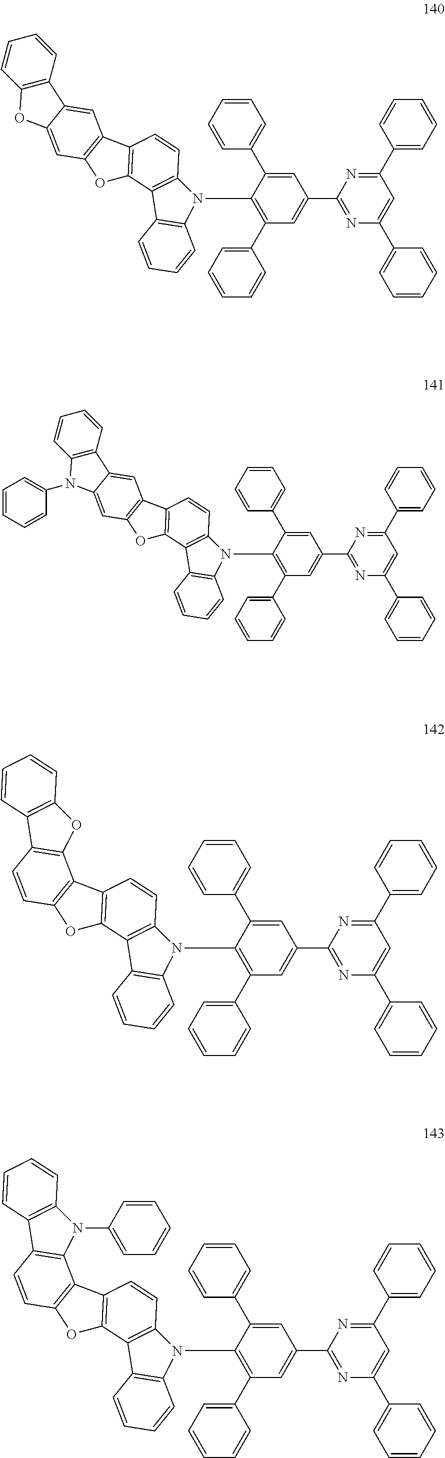 [SMILES: c1ccc(-c2cc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1]

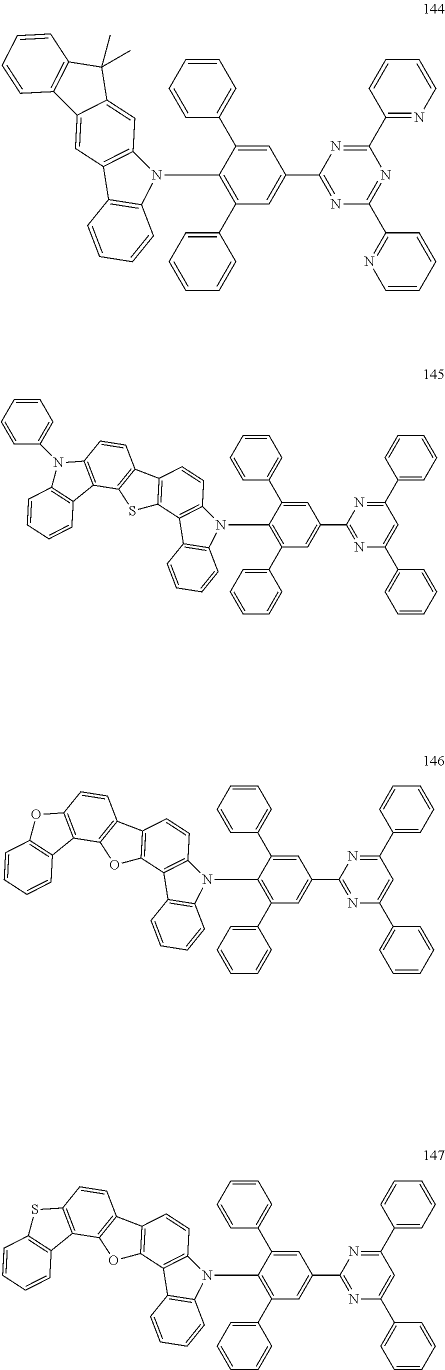 [SMILES: CC1(C)c2ccccc2-c2cc3c4ccccc4n(-c4c(-c5ccccc5)cc(-c5nc(-c6ccccn6)nc(-c6ccccn6)n5)cc4-c4ccccc4)c3cc21 ; c1ccc(-c2cc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1]

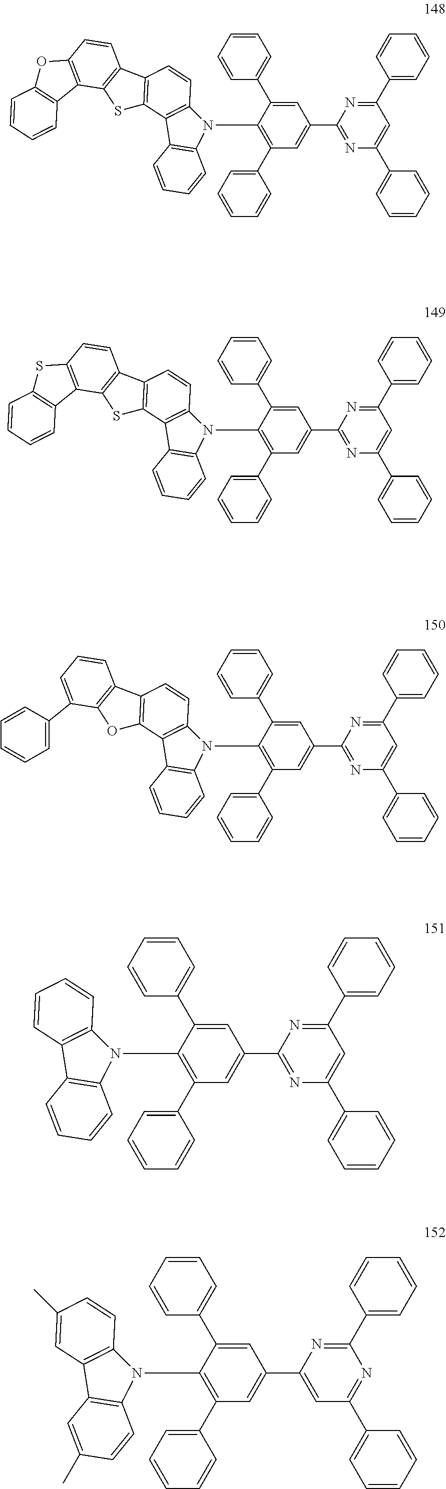

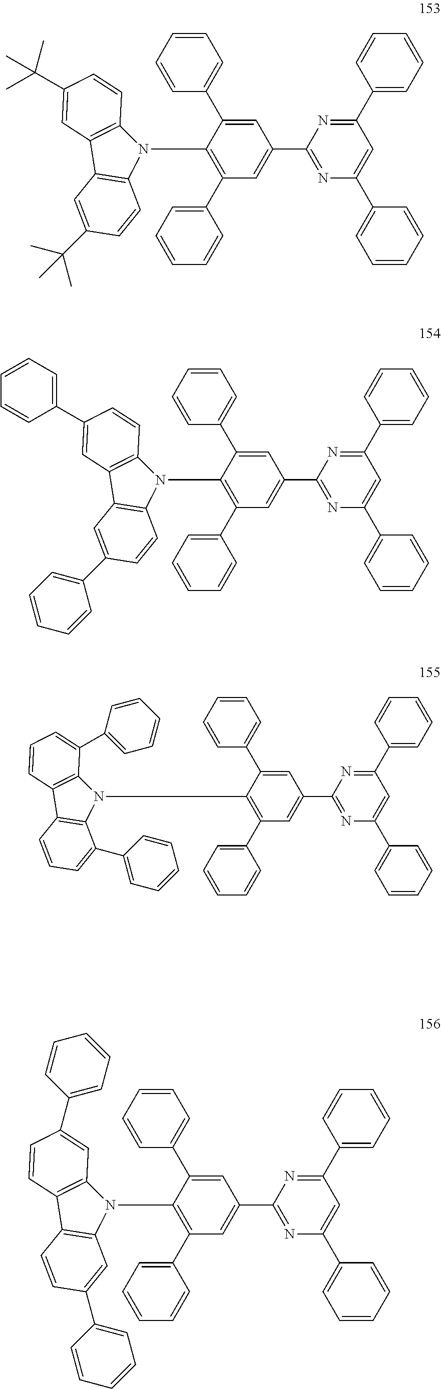 [SMILES: CC(C)(C)c1ccc2c(c1)c1cc(C(C)(C)C)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)cc(-c4ccccc4)n3)cc2-c2ccccc2)cc1 ; c1ccc(-c2cc(-c3ccccc3)nc(-c3cc(-c4ccccc4)c(-n4c5c(-c6ccccc6)cccc5c5cccc(-c6ccccc6)c54)c(-c4ccccc4)c3)n2)cc1 ; c1ccc(-c2ccc3c4ccc(-c5ccccc5)cc4n(-c4c(-c5ccccc5)cc(-c5nc(-c6ccccc6)cc(-c6ccccc6)n5)cc4-c4ccccc4)c3c2)cc1]

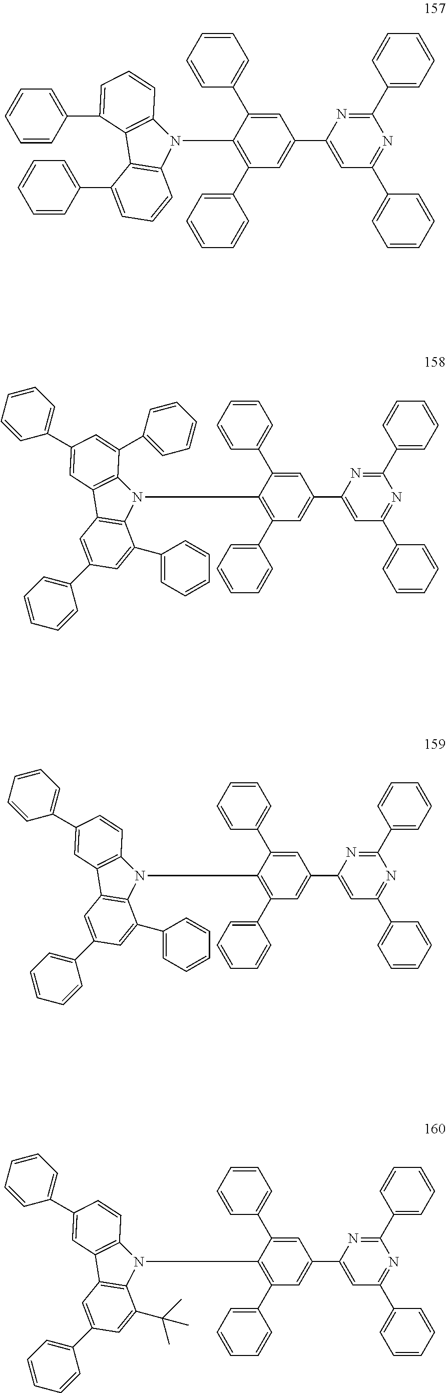 [SMILES: c1ccc(-c2cc(-c3cc(-c4ccccc4)c(-n4c5cccc(-c6ccccc6)c5c5c(-c6ccccc6)cccc54)c(-c4ccccc4)c3)nc(-c3ccccc3)n2)cc1 ; c1ccc(-c2cc(-c3ccccc3)c3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4cc(-c5ccccc5)nc(-c5ccccc5)n4)cc3-c3ccccc3)c12]

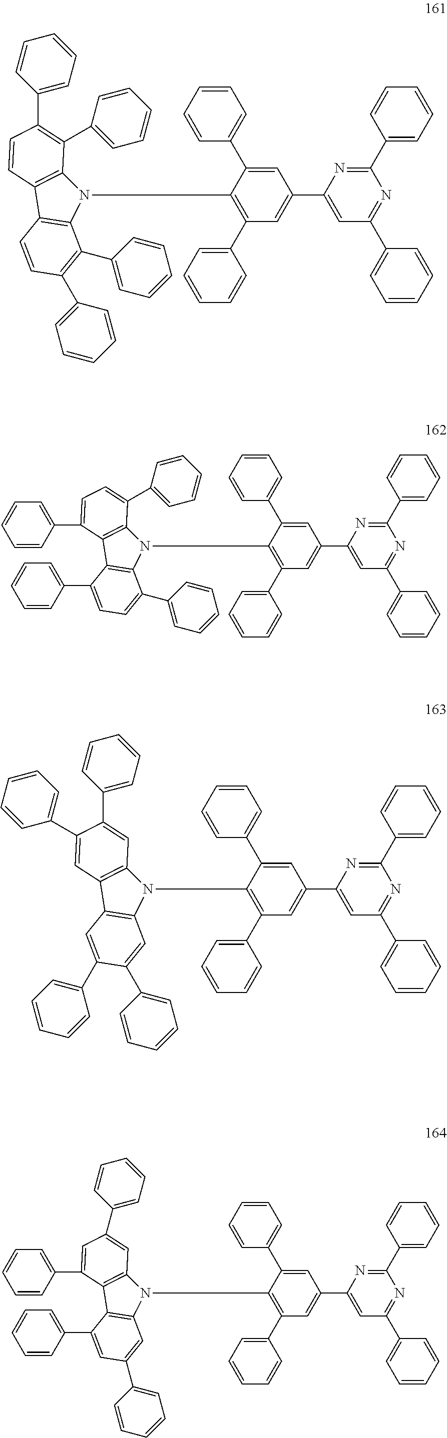

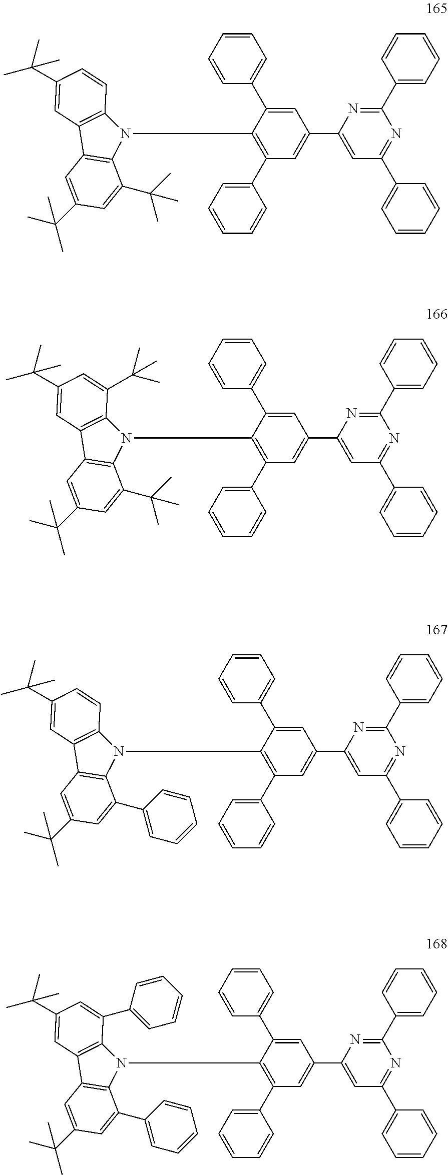 [SMILES: CC(C)(C)c1ccc2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1 ; CC(C)(C)c1ccc2c(c1)c1cc(C(C)(C)C)cc(-c3ccccc3)c1n2-c1c(-c2ccccc2)cc(-c2cc(-c3ccccc3)nc(-c3ccccc3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2cc(-c3ccccc3)nc(-c3ccccc3)n2)cc1-c1ccccc1]

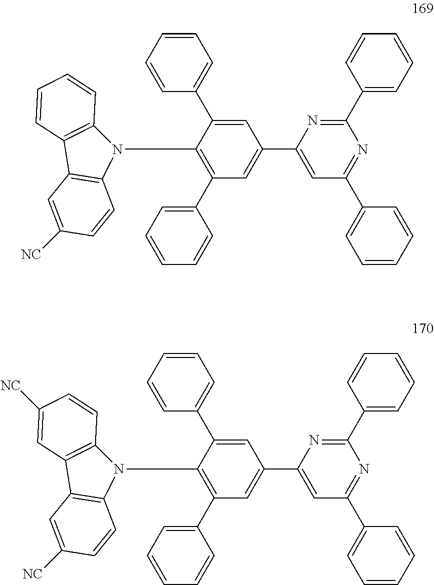 [SMILES: N#Cc1ccc2c(c1)c1ccccc1n2-c1c(-c2ccccc2)cc(-c2cc(-c3ccccc3)nc(-c3ccccc3)n2)cc1-c1ccccc1 ; N#Cc1ccc2c(c1)c1cc(C#N)ccc1n2-c1c(-c2ccccc2)cc(-c2cc(-c3ccccc3)nc(-c3ccccc3)n2)cc1-c1ccccc1]

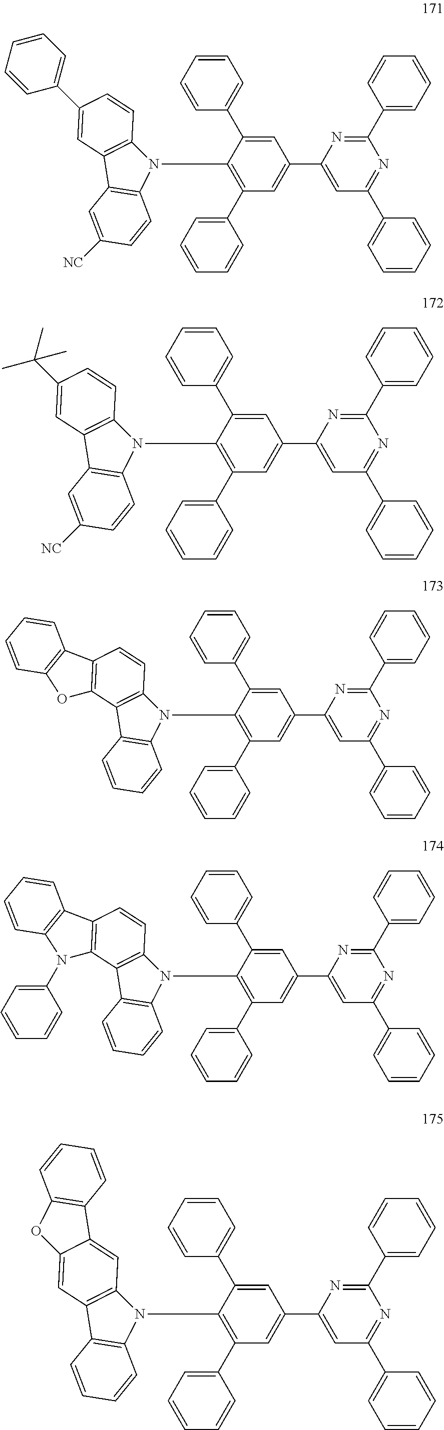

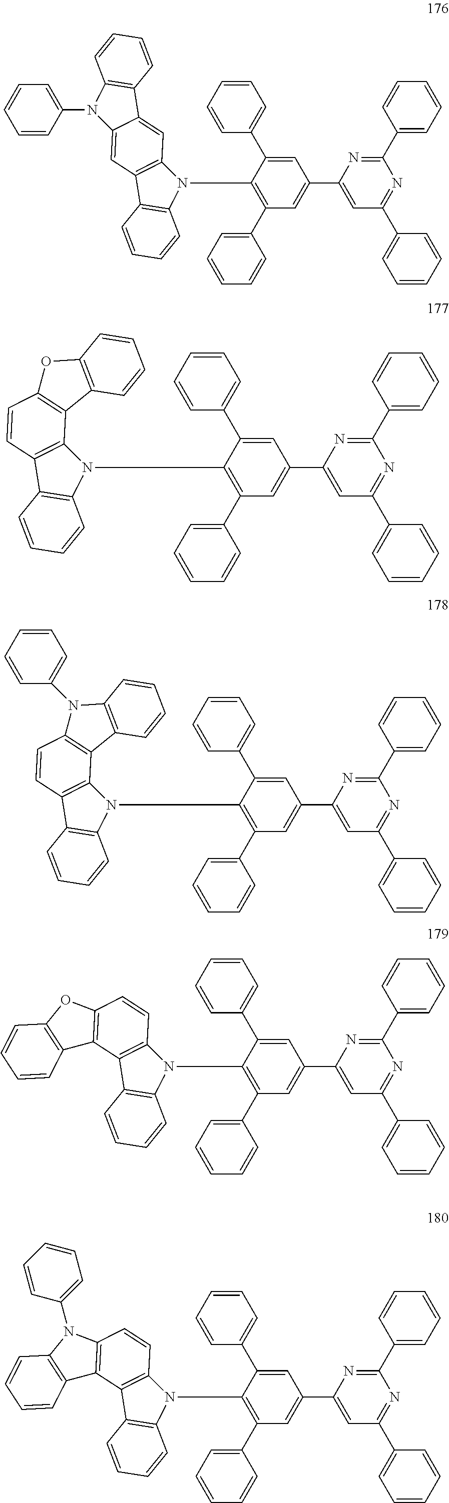

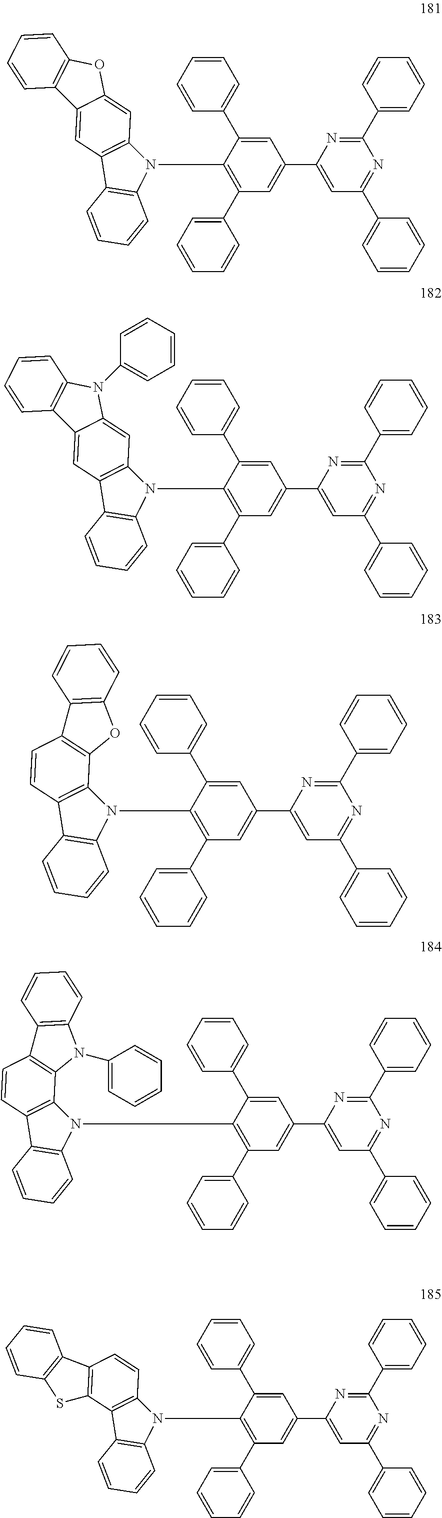

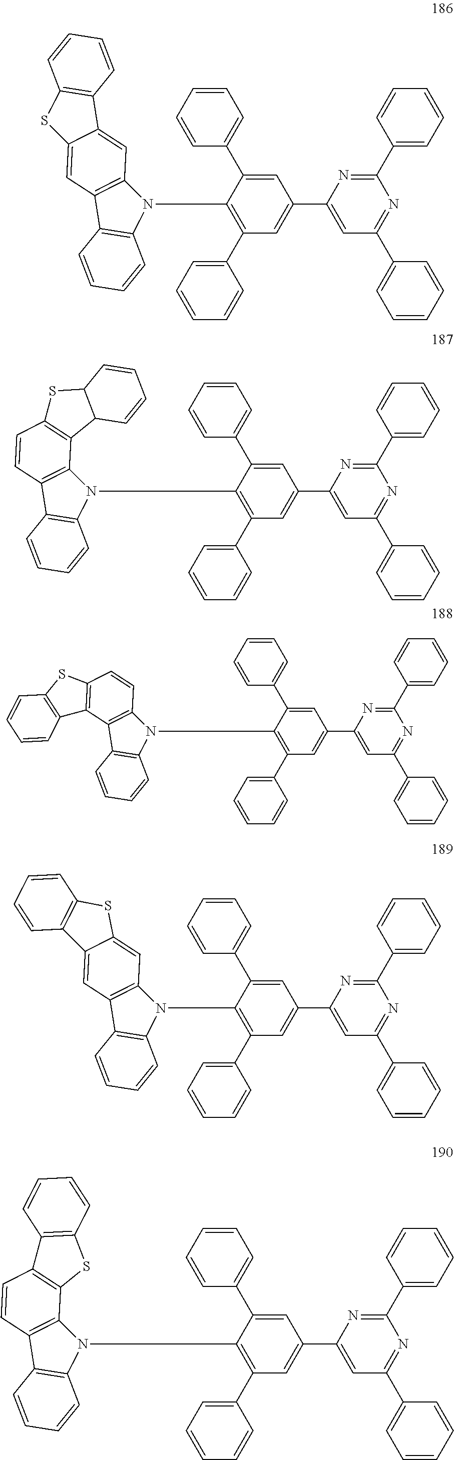

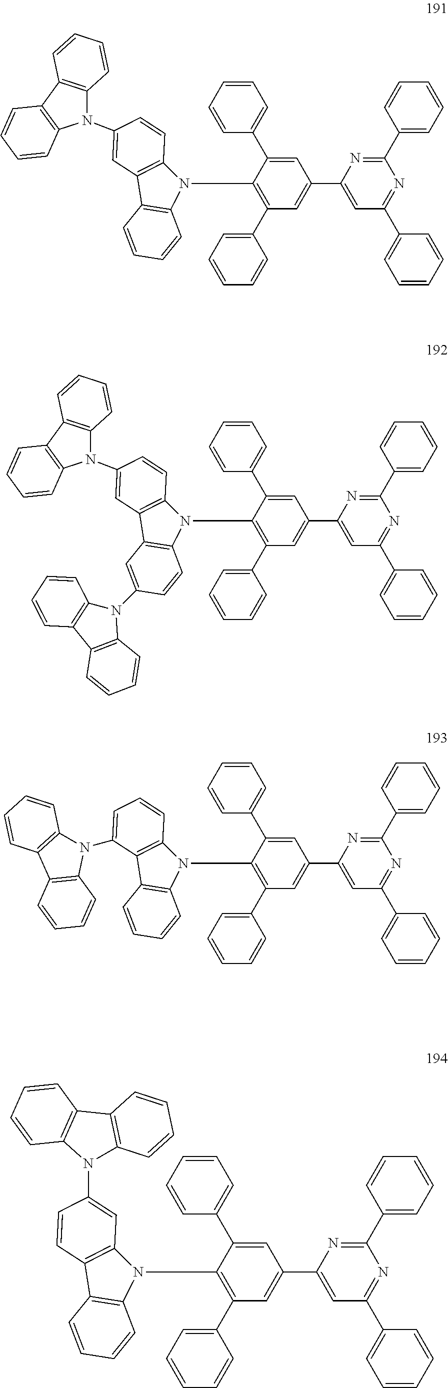 [SMILES: c1ccc(-c2cc(-c3cc(-c4ccccc4)c(-n4c5ccccc5c5cc(-n6c7ccccc7c7ccccc76)ccc54)c(-c4ccccc4)c3)nc(-c3ccccc3)n2)cc1 ; c1ccc(-c2cc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccccc3)n2)cc1 ; c1ccc(-c2cc(-c3cc(-c4ccccc4)c(-n4c5ccccc5c5c(-n6c7ccccc7c7ccccc76)cccc54)c(-c4ccccc4)c3)nc(-c3ccccc3)n2)cc1 ; c1ccc(-c2cc(-c3cc(-c4ccccc4)c(-n4c5ccccc5c5ccc(-n6c7ccccc7c7ccccc76)cc54)c(-c4ccccc4)c3)nc(-c3ccccc3)n2)cc1]

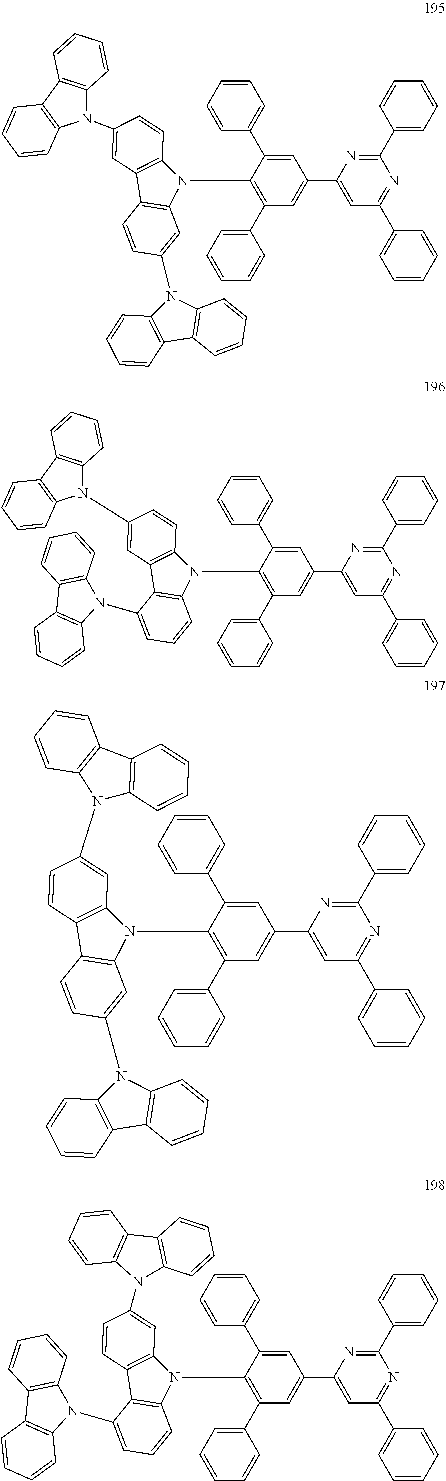

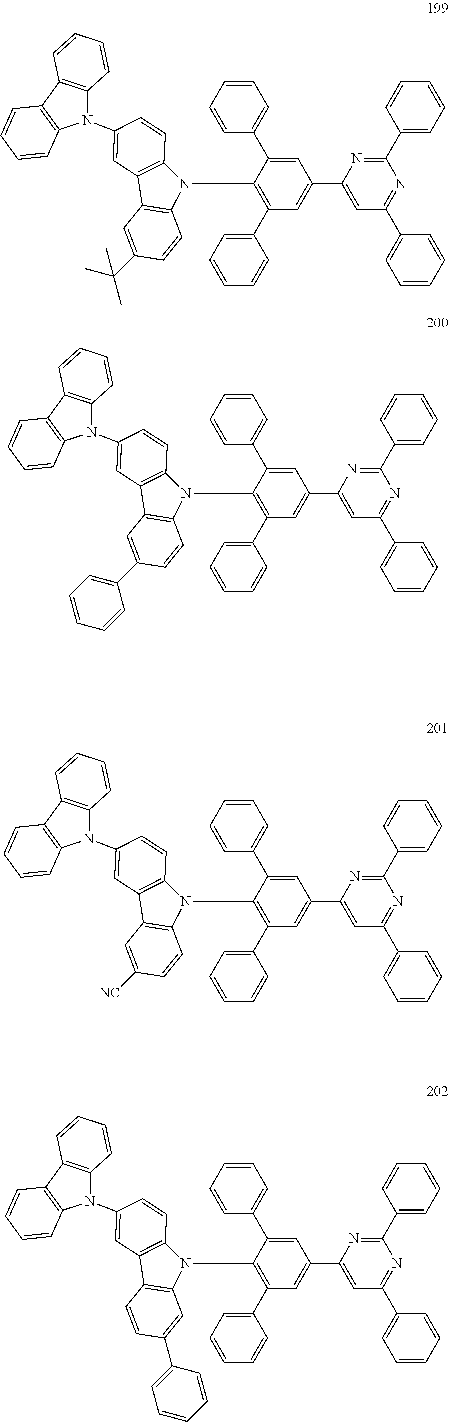 [SMILES: CC(C)(C)c1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3cc(-c4ccccc4)nc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5cc(-c6ccccc6)nc(-c6ccccc6)n5)cc4-c4ccccc4)c3c2)cc1]

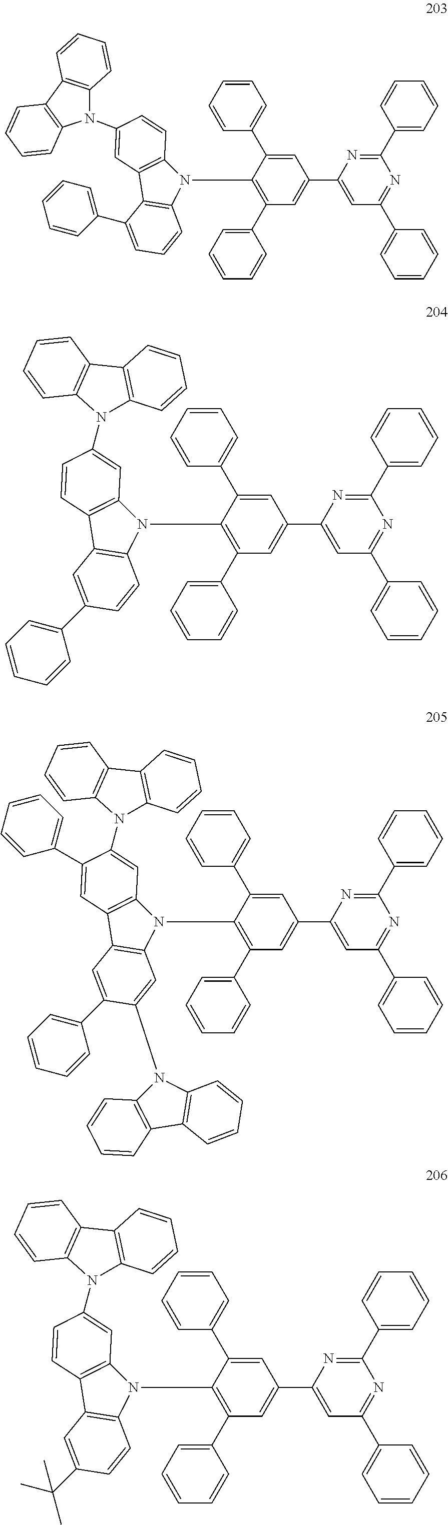

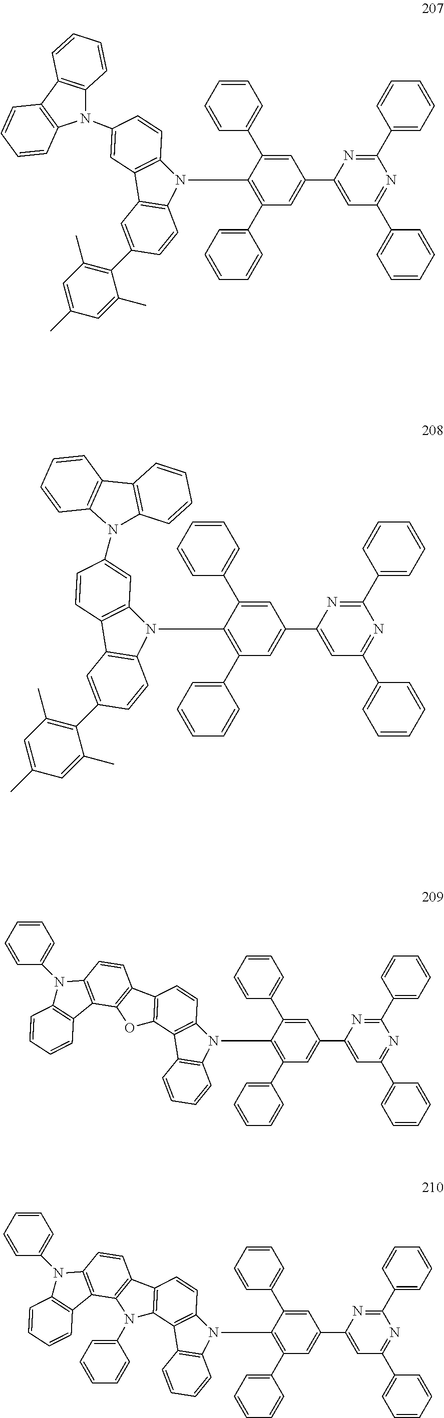

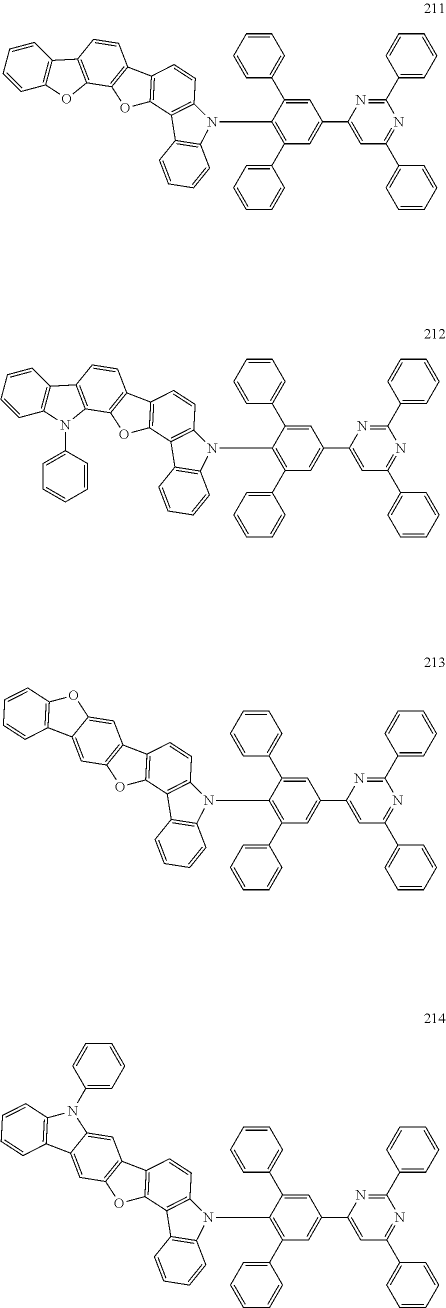 [SMILES: c1ccc(-c2cc(-c3cc(-c4ccccc4)c(-n4c5ccccc5c5c6oc7c(ccc8c9ccccc9oc87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(ccc8c9ccccc9n(-c9ccccc9)c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8c(cc7c6ccc54)oc4ccccc48)c(-c4ccccc4)c3)nc(-c3ccccc3)n2)cc1 ; c1ccc(-c2cc(-c3cc(-c4ccccc4)c(-n4c5ccccc5c5c6oc7cc8c9ccccc9n(-c9ccccc9)c8cc7c6ccc54)c(-c4ccccc4)c3)nc(-c3ccccc3)n2)cc1]

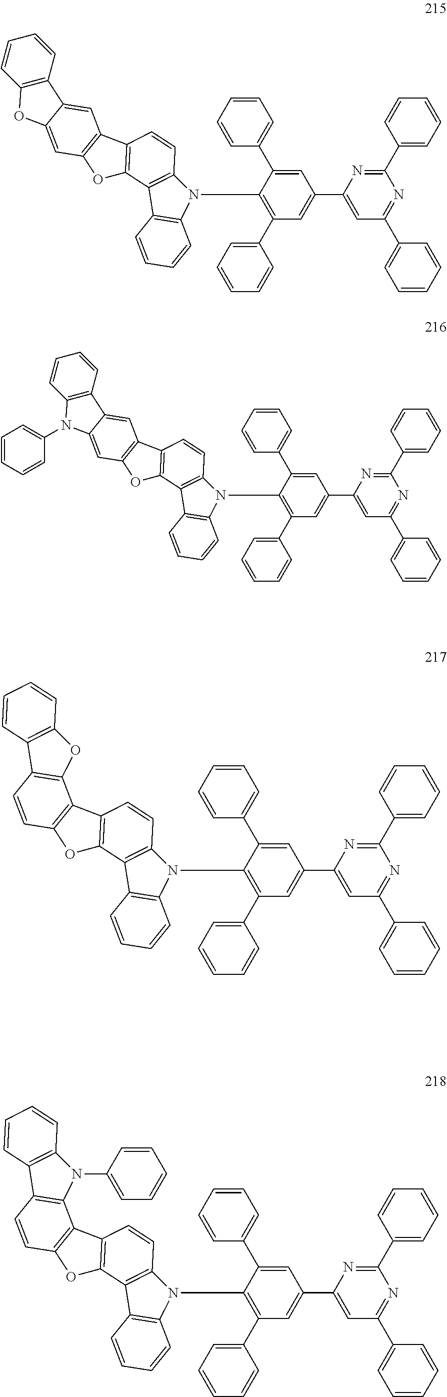 [SMILES: c1ccc(-c2cc(-c3cc(-c4ccccc4)c(-n4c5ccccc5c5c6oc7cc8oc9ccccc9c8cc7c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8c(cc7c6ccc54)c4ccccc4n8-c4ccccc4)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9oc8c7c6ccc54)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9n(-c9ccccc9)c8c7c6ccc54)c(-c4ccccc4)c3)nc(-c3ccccc3)n2)cc1]

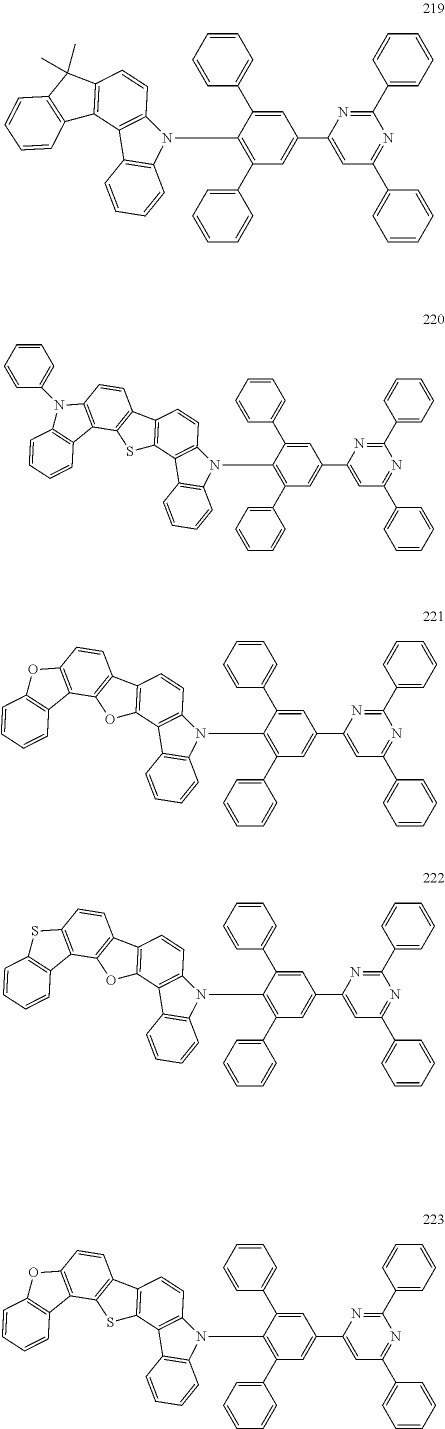

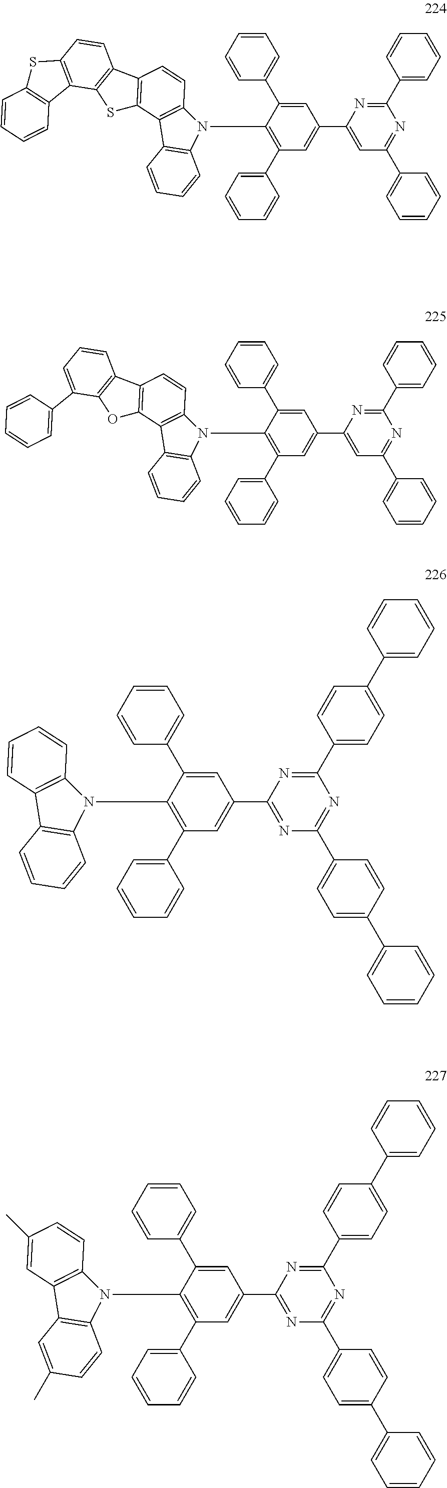 [SMILES: c1ccc(-c2cc(-c3cc(-c4ccccc4)c(-n4c5ccccc5c5c6sc7c(ccc8sc9ccccc9c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(-c8ccccc8)cccc7c6ccc54)c(-c4ccccc4)c3)nc(-c3ccccc3)n2)cc1 ; c1ccc(-c2ccc(-c3nc(-c4ccc(-c5ccccc5)cc4)nc(-c4cc(-c5ccccc5)c(-n5c6ccccc6c6ccccc65)c(-c5ccccc5)c4)n3)cc2)cc1 ; Cc1ccc2c(c1)c1cc(C)ccc1n2-c1c(-c2ccccc2)cc(-c2nc(-c3ccc(-c4ccccc4)cc3)nc(-c3ccc(-c4ccccc4)cc3)n2)cc1-c1ccccc1]

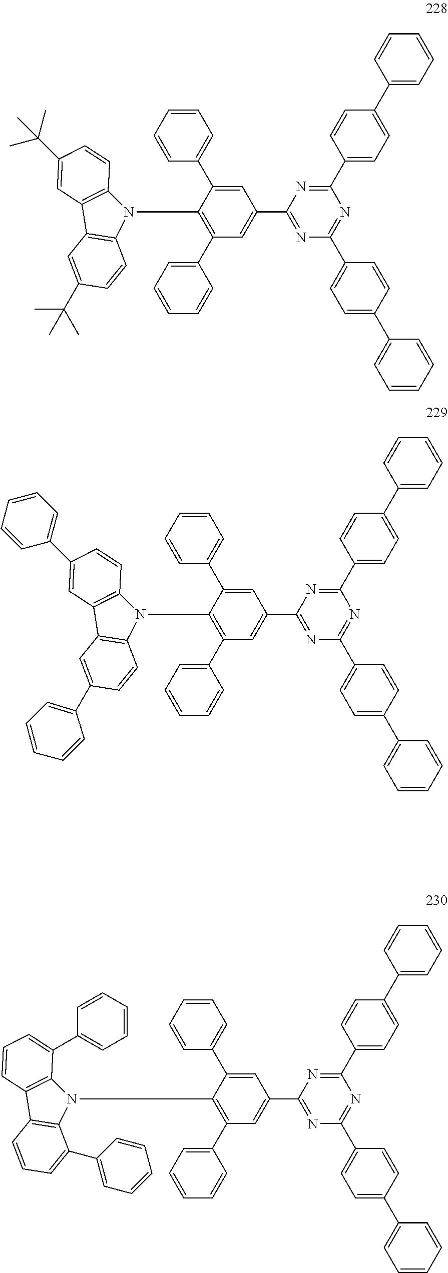 [SMILES: CC(C)(C)c1ccc2c(c1)c1cc(C(C)(C)C)ccc1n2-c1c(-c2ccccc2)cc(-c2nc(-c3ccc(-c4ccccc4)cc3)nc(-c3ccc(-c4ccccc4)cc3)n2)cc1-c1ccccc1 ; c1ccc(-c2ccc(-c3nc(-c4ccc(-c5ccccc5)cc4)nc(-c4cc(-c5ccccc5)c(-n5c6ccc(-c7ccccc7)cc6c6cc(-c7ccccc7)ccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6c(-c7ccccc7)cccc6c6cccc(-c7ccccc7)c65)c(-c5ccccc5)c4)n3)cc2)cc1]

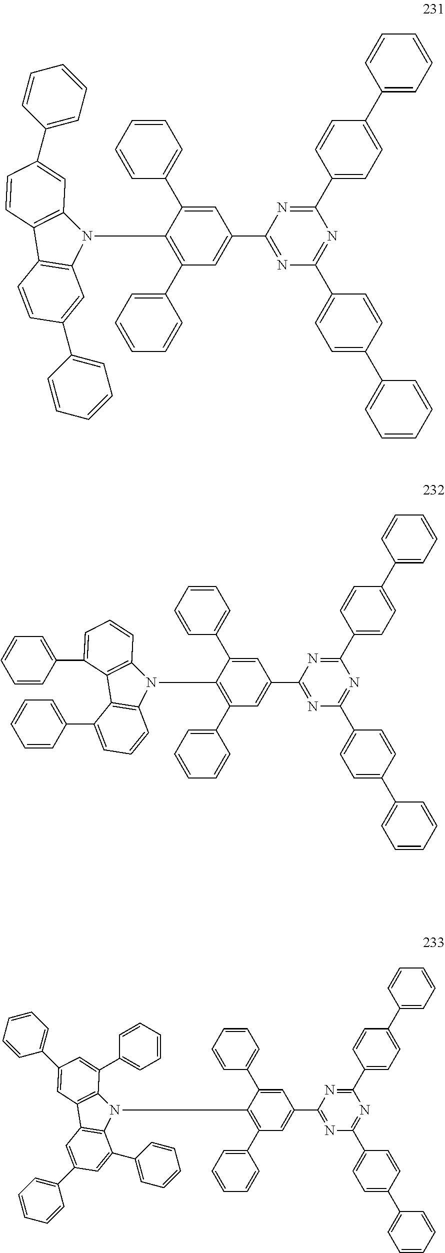 [SMILES: c1ccc(-c2ccc(-c3nc(-c4ccc(-c5ccccc5)cc4)nc(-c4cc(-c5ccccc5)c(-n5c6cc(-c7ccccc7)ccc6c6ccc(-c7ccccc7)cc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6cccc(-c7ccccc7)c6c6c(-c7ccccc7)cccc65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1]

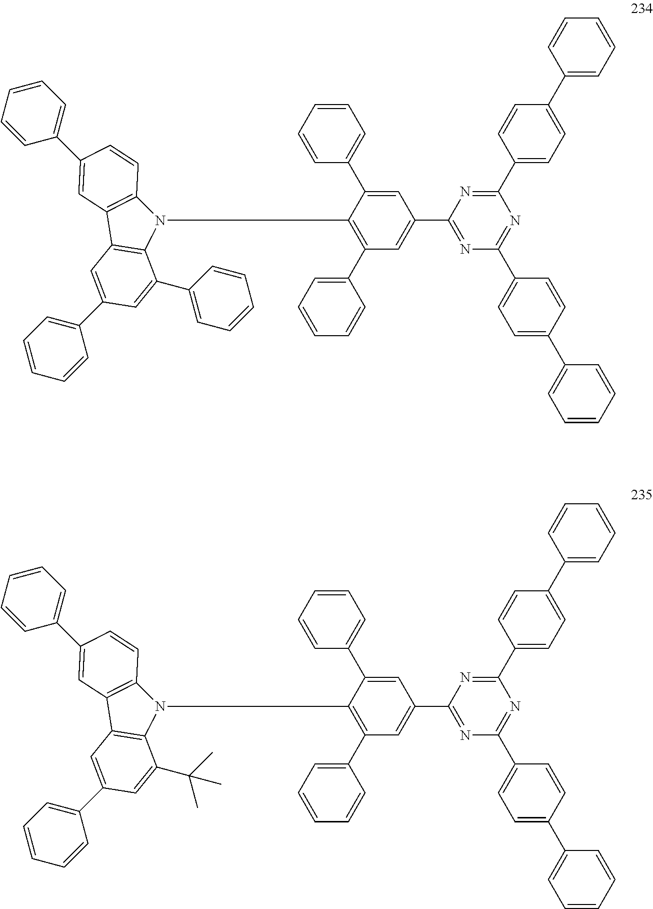 [SMILES: c1ccc(-c2ccc(-c3nc(-c4ccc(-c5ccccc5)cc4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1 ; CC(C)(C)c1cc(-c2ccccc2)cc2c3cc(-c4ccccc4)ccc3n(-c3c(-c4ccccc4)cc(-c4nc(-c5ccc(-c6ccccc6)cc5)nc(-c5ccc(-c6ccccc6)cc5)n4)cc3-c3ccccc3)c12]

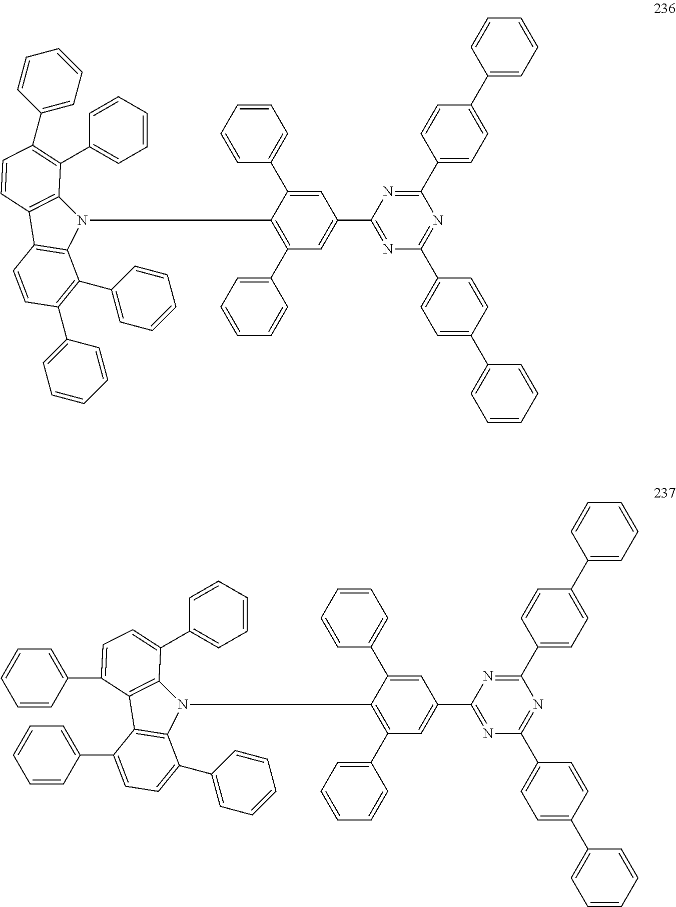 [SMILES: c1ccc(-c2ccc(-c3nc(-c4ccc(-c5ccccc5)cc4)nc(-c4cc(-c5ccccc5)c(-n5c6c(-c7ccccc7)c(-c7ccccc7)ccc6c6ccc(-c7ccccc7)c(-c7ccccc7)c65)c(-c5ccccc5)c4)n3)cc2)cc1 ; c1ccc(-c2ccc(-c3nc(-c4ccc(-c5ccccc5)cc4)nc(-c4cc(-c5ccccc5)c(-n5c6c(-c7ccccc7)ccc(-c7ccccc7)c6c6c(-c7ccccc7)ccc(-c7ccccc7)c65)c(-c5ccccc5)c4)n3)cc2)cc1]

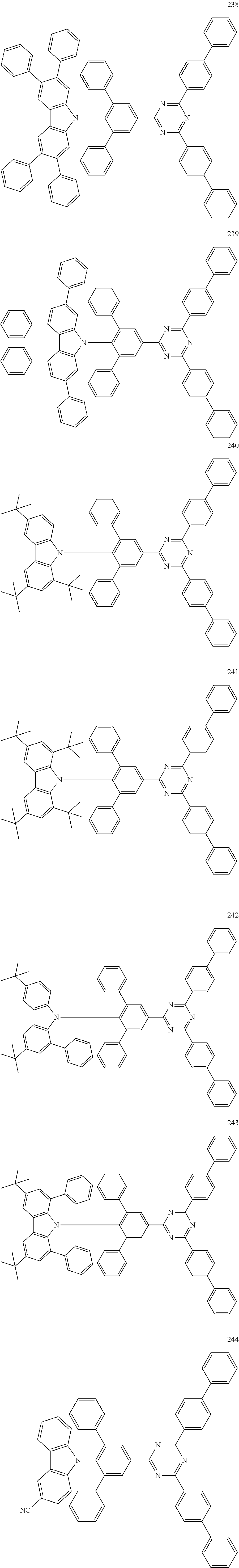

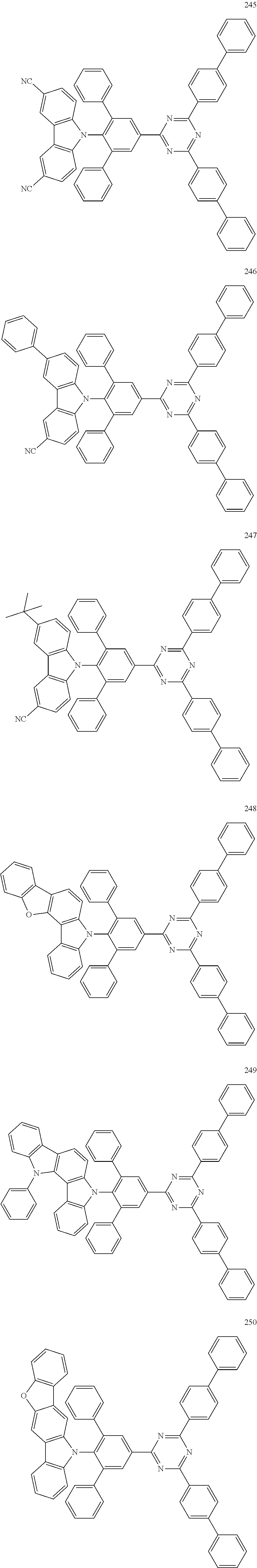

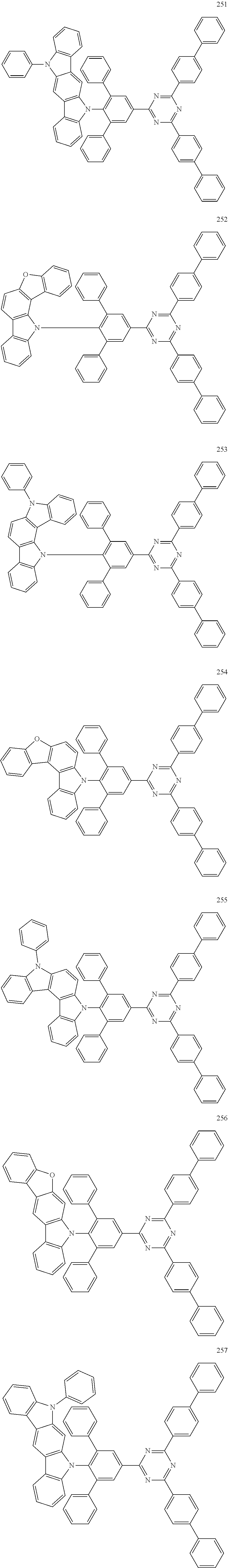

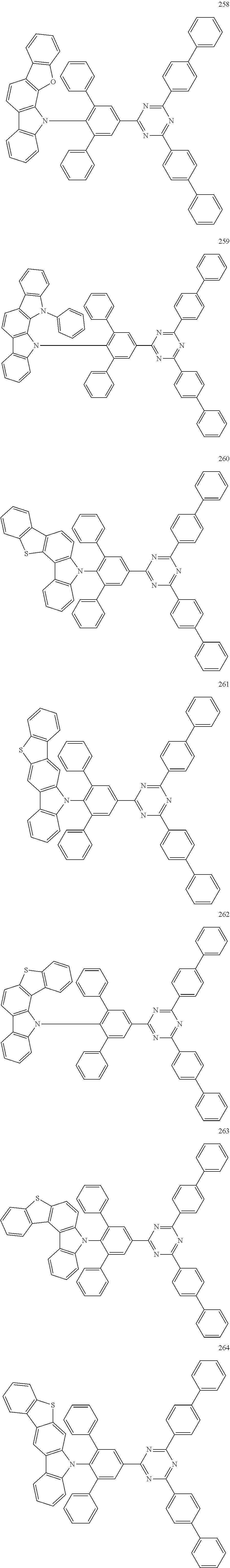

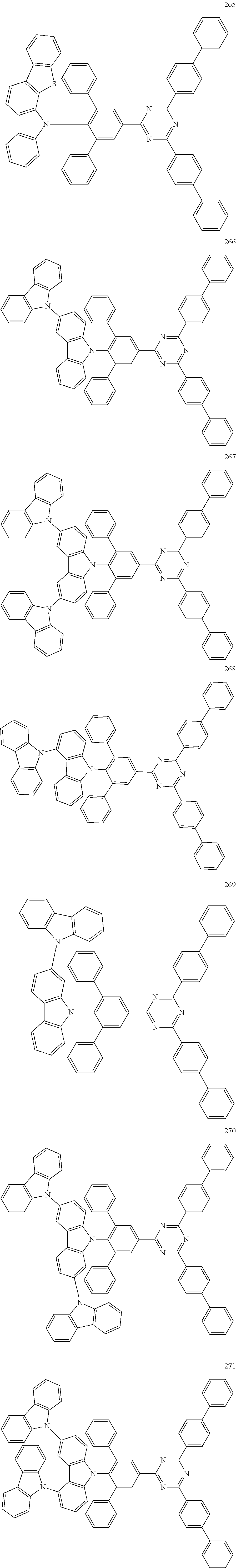

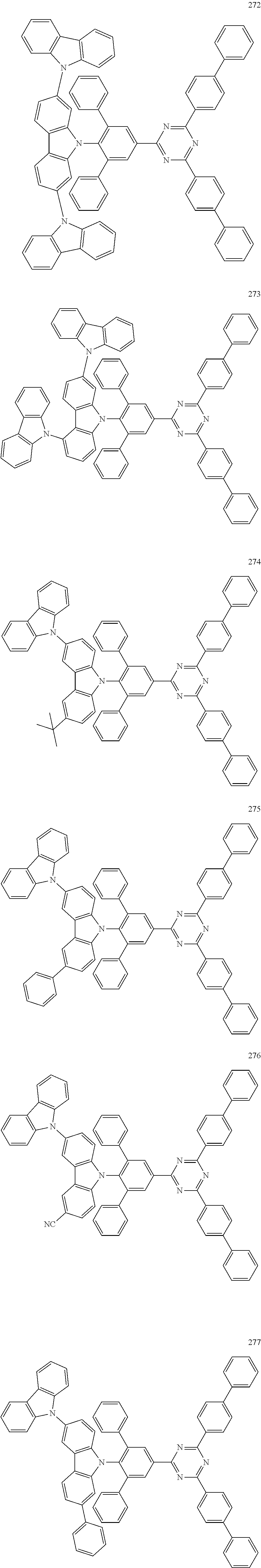

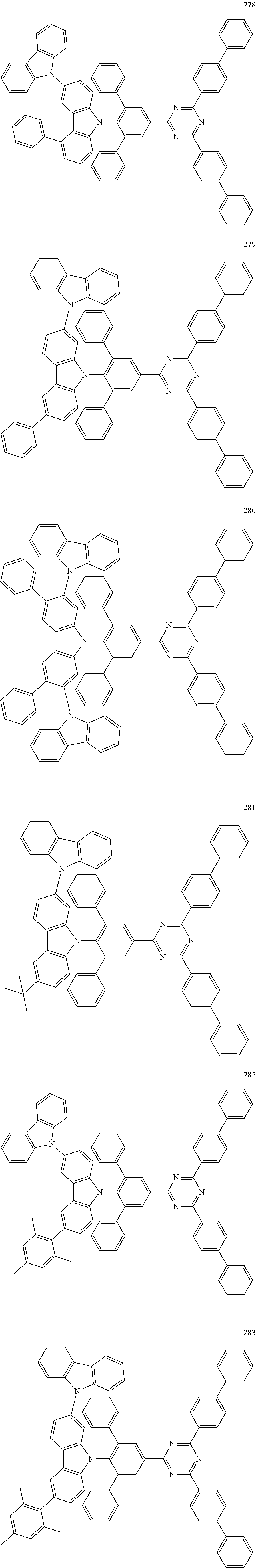

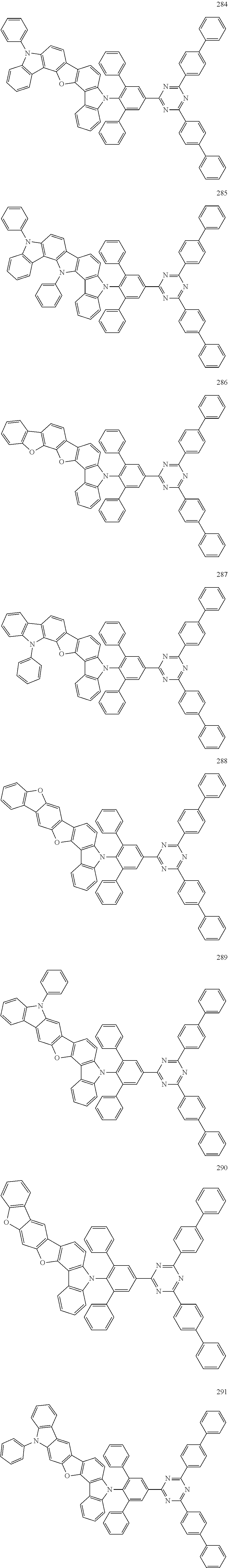

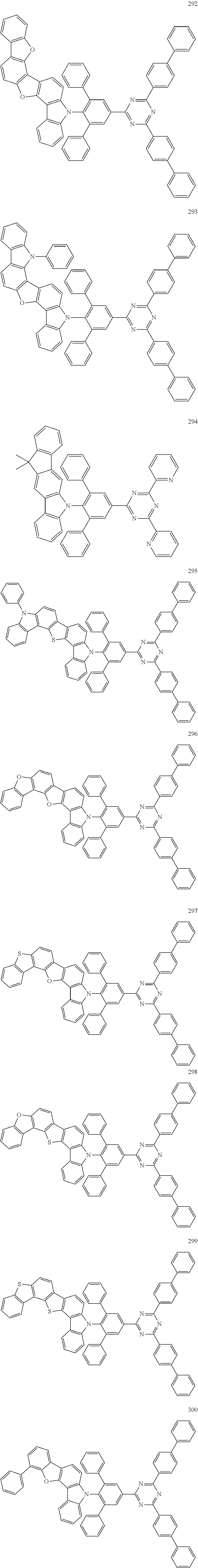

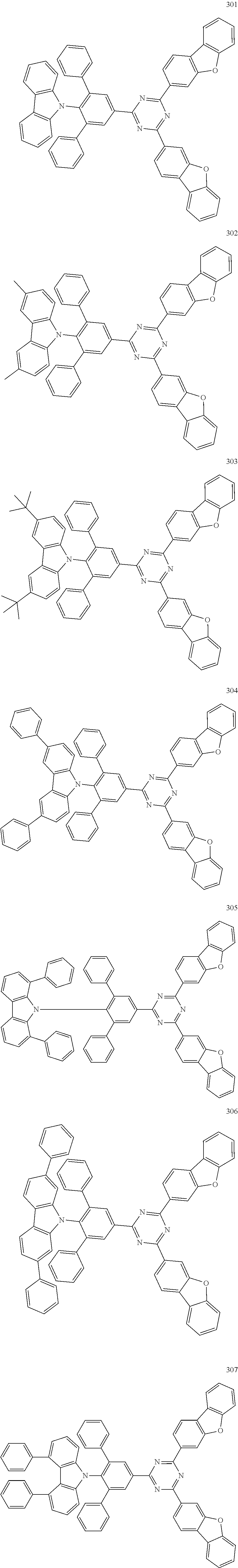

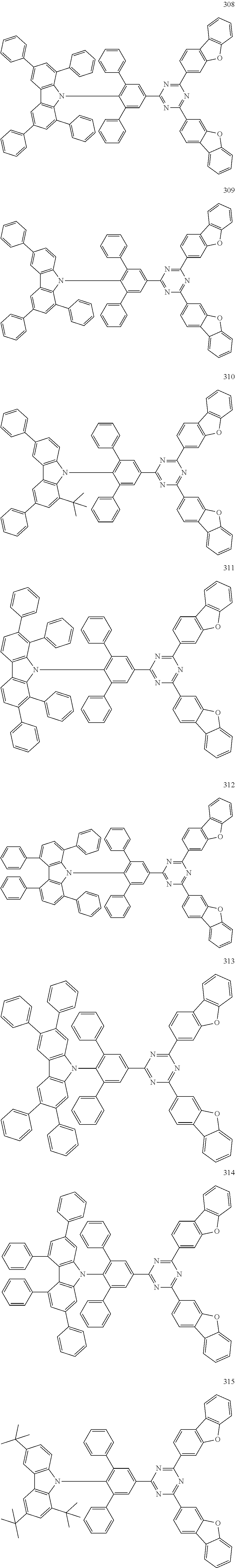

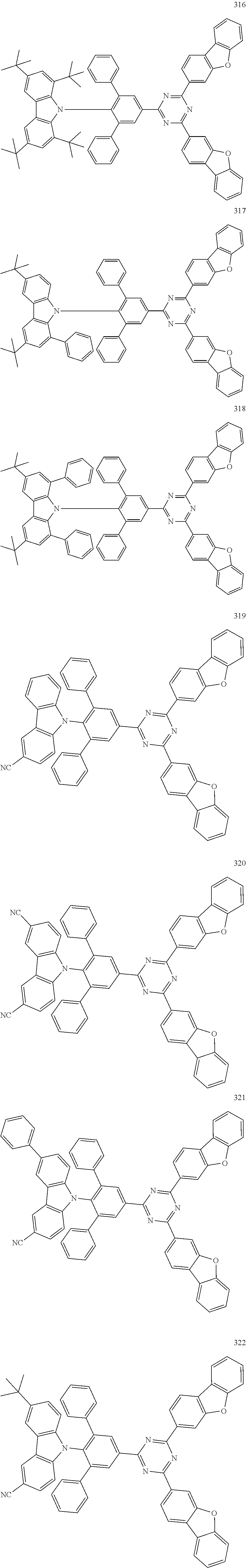

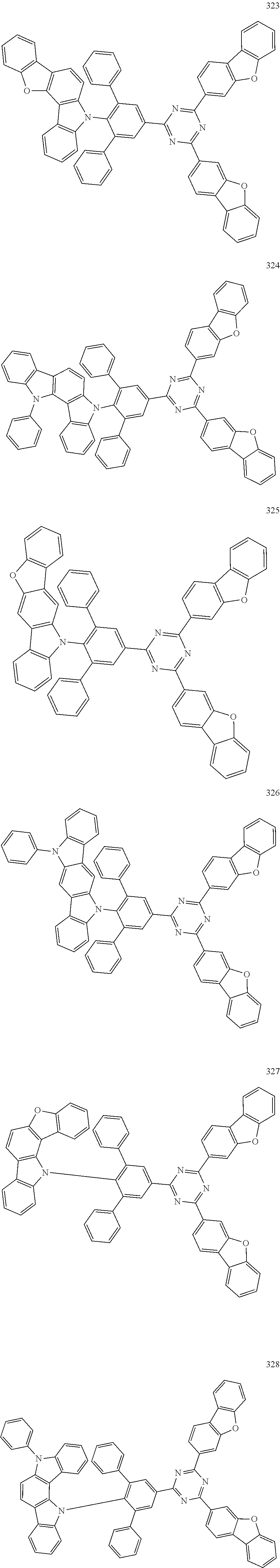

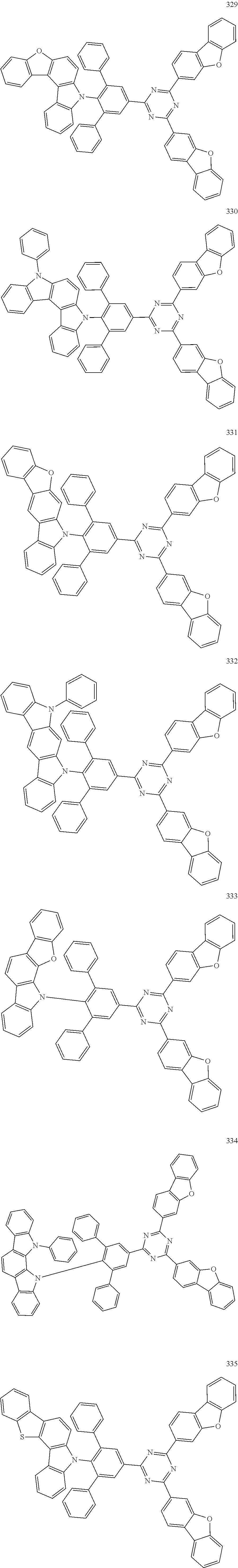

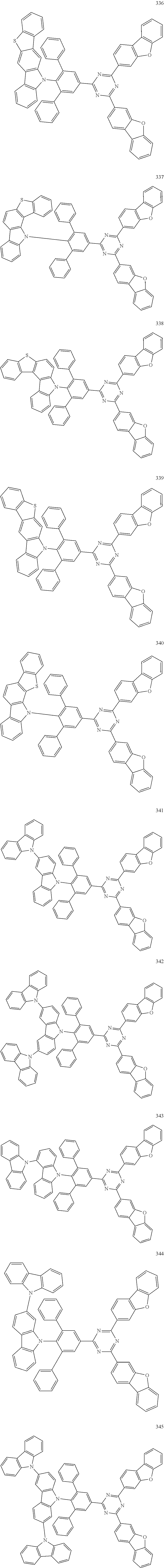

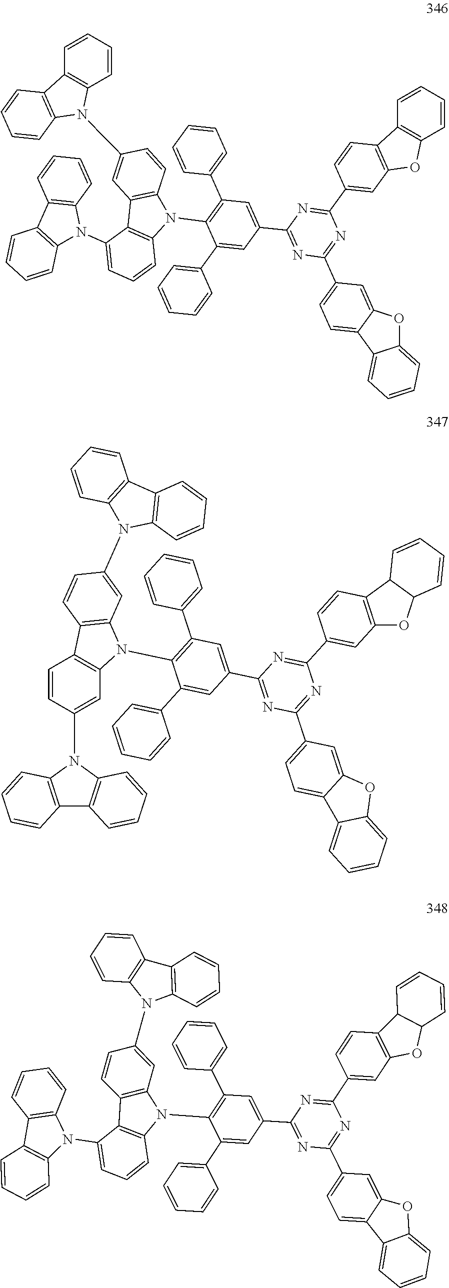 [SMILES: c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-n4c5ccccc5c5ccccc54)cc3c3c(-n4c5ccccc5c5ccccc54)cccc32)cc1 ; C1=CC2Oc3cc(-c4nc(-c5cc(-c6ccccc6)c(-n6c7cc(-n8c9ccccc9c9ccccc98)ccc7c7ccc(-n8c9ccccc9c9ccccc98)cc76)c(-c6ccccc6)c5)nc(-c5ccc6c(c5)oc5ccccc56)n4)ccc3C2C=C1 ; C1=CC2Oc3cc(-c4nc(-c5cc(-c6ccccc6)c(-n6c7cc(-n8c9ccccc9c9ccccc98)ccc7c7c(-n8c9ccccc9c9ccccc98)cccc76)c(-c6ccccc6)c5)nc(-c5ccc6c(c5)oc5ccccc56)n4)ccc3C2C=C1]

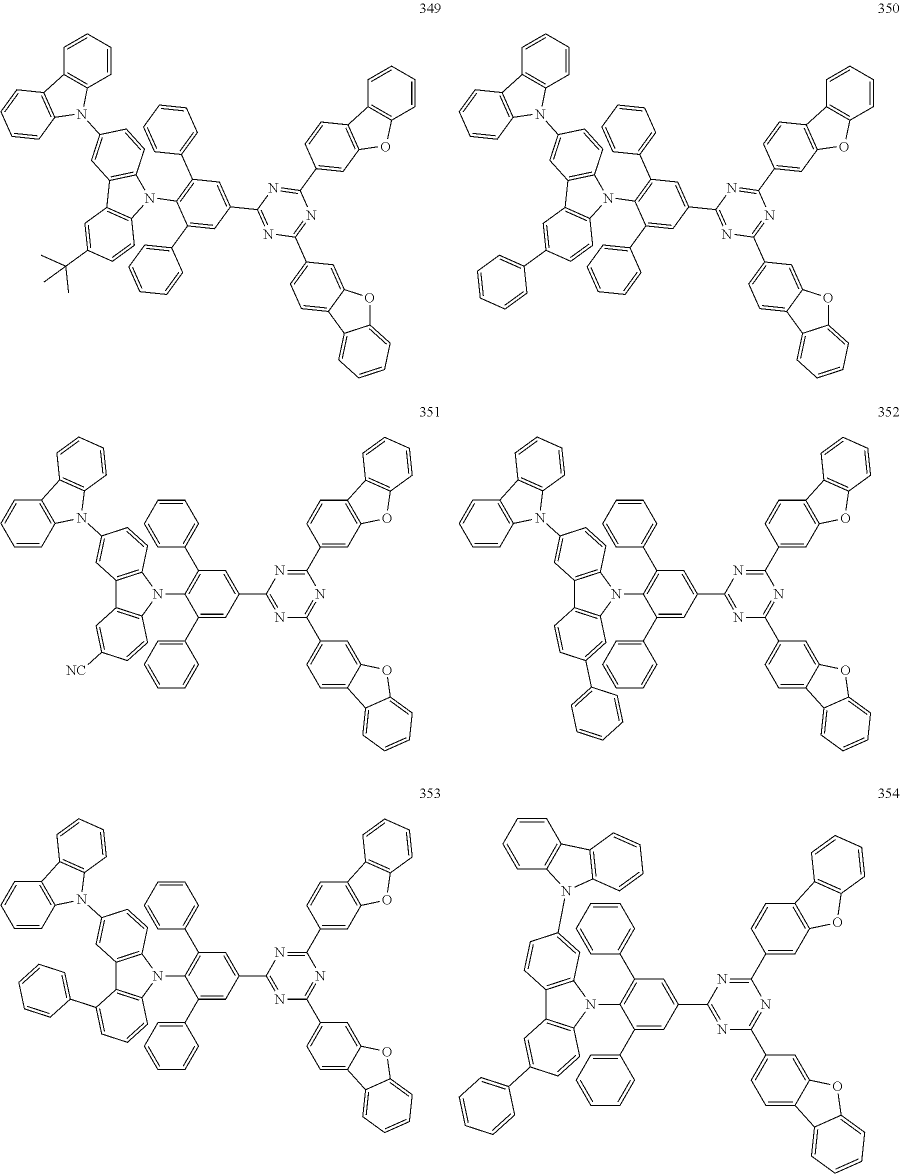

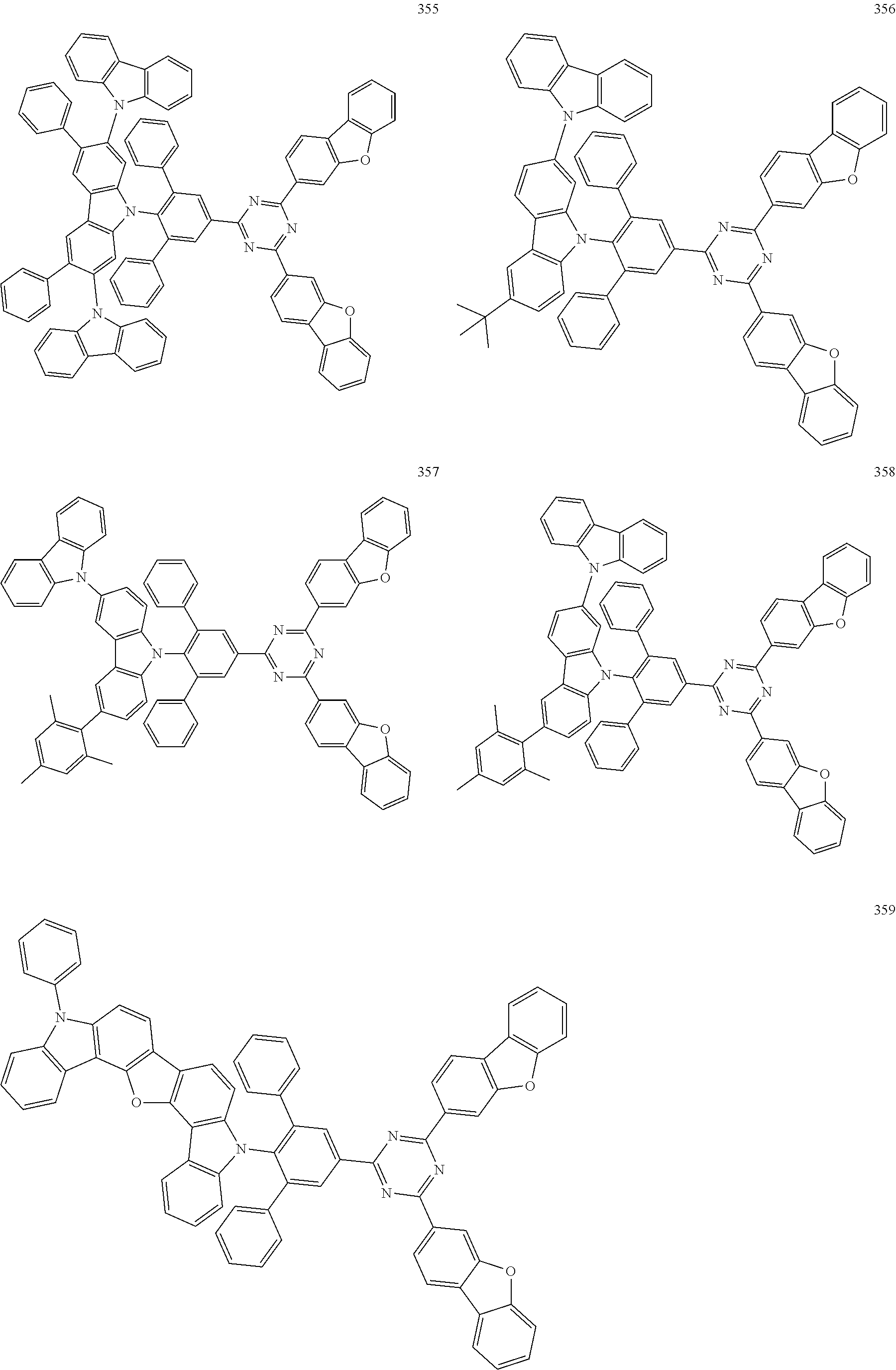

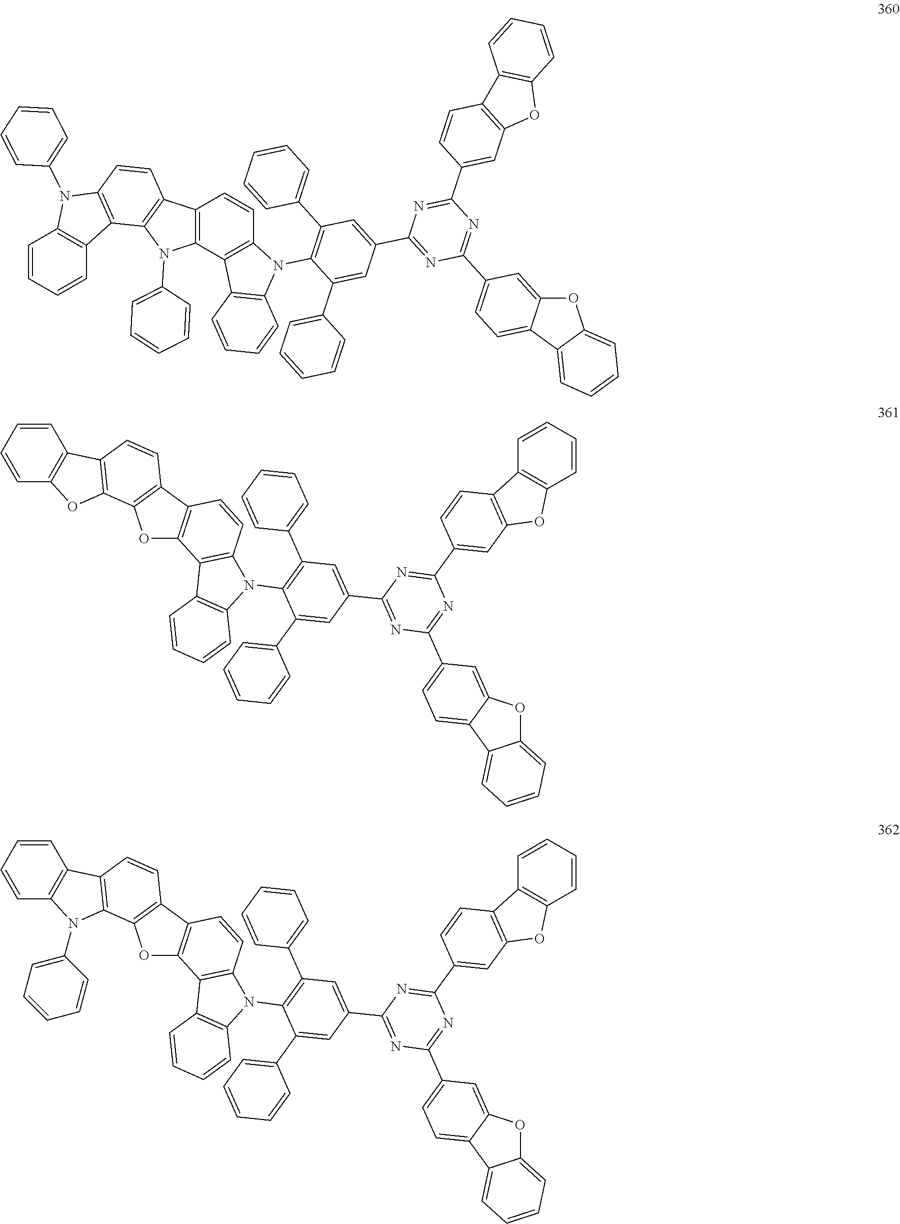

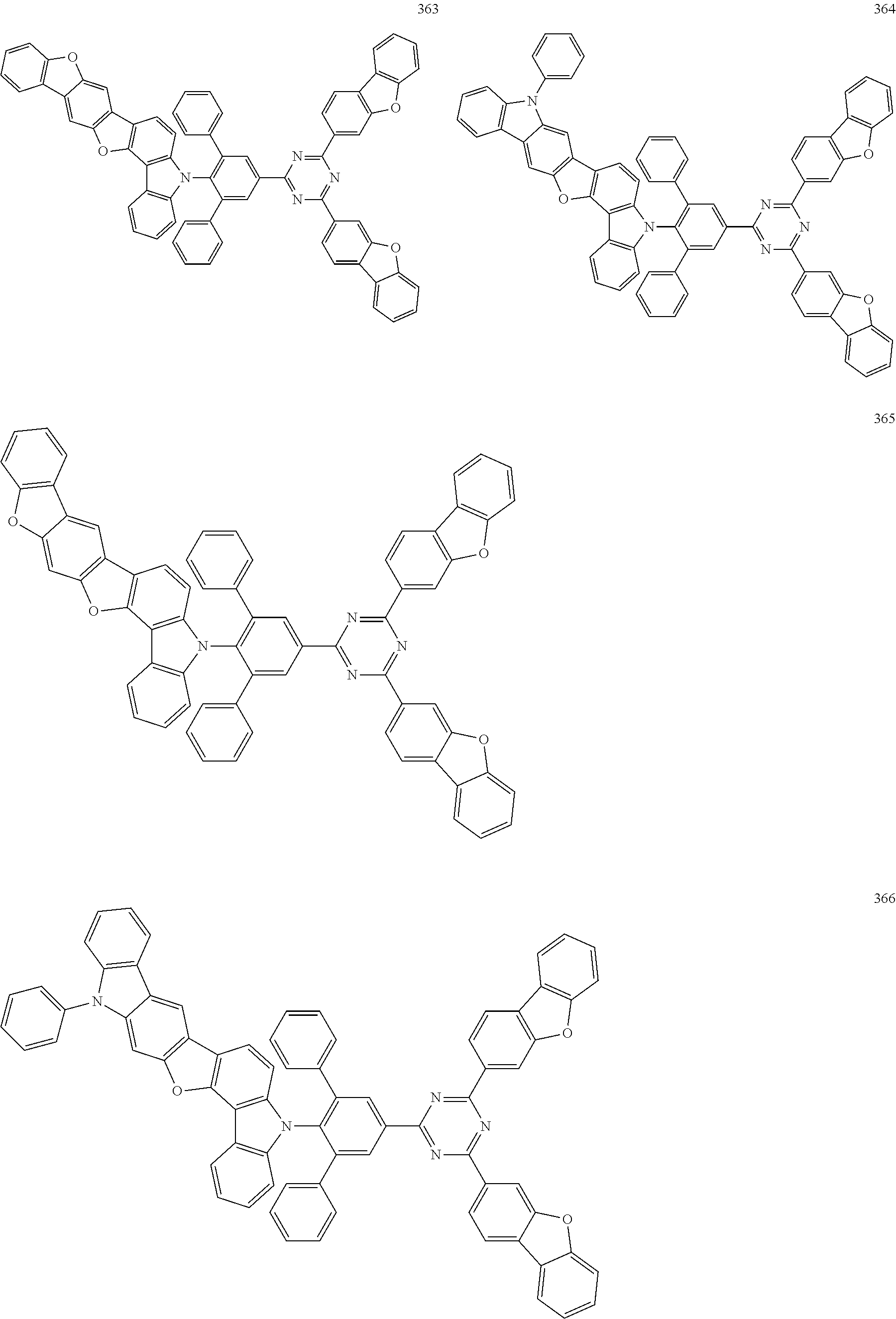

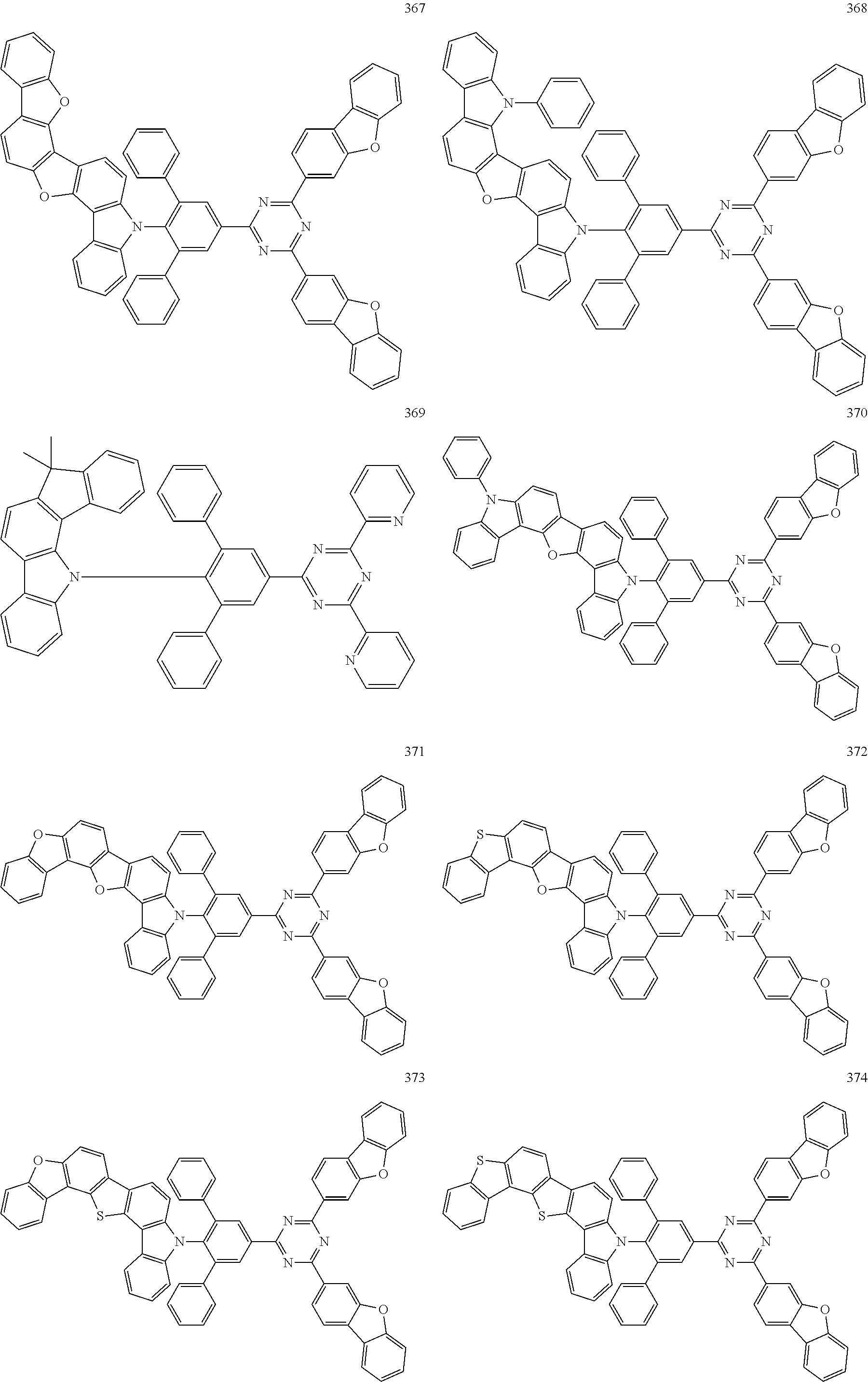

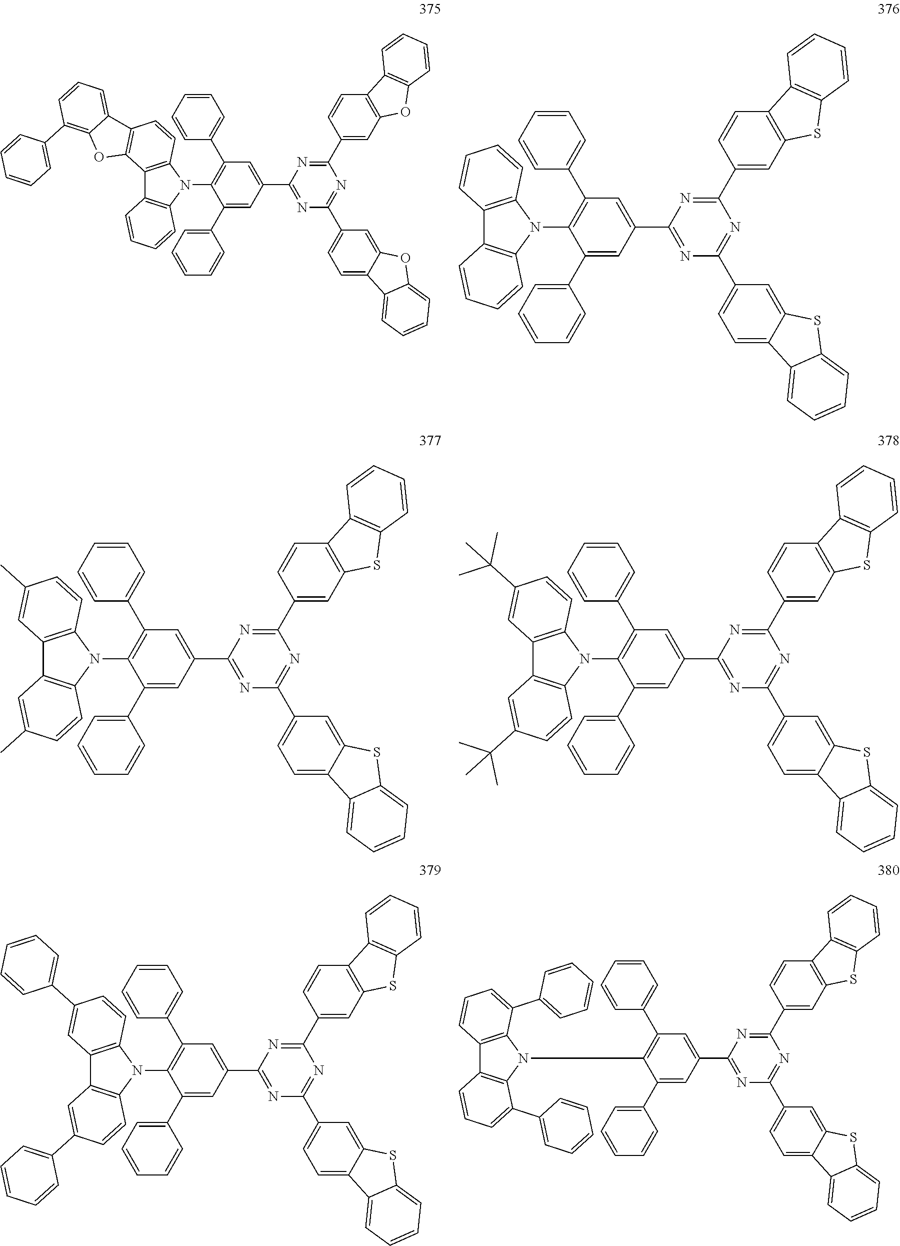

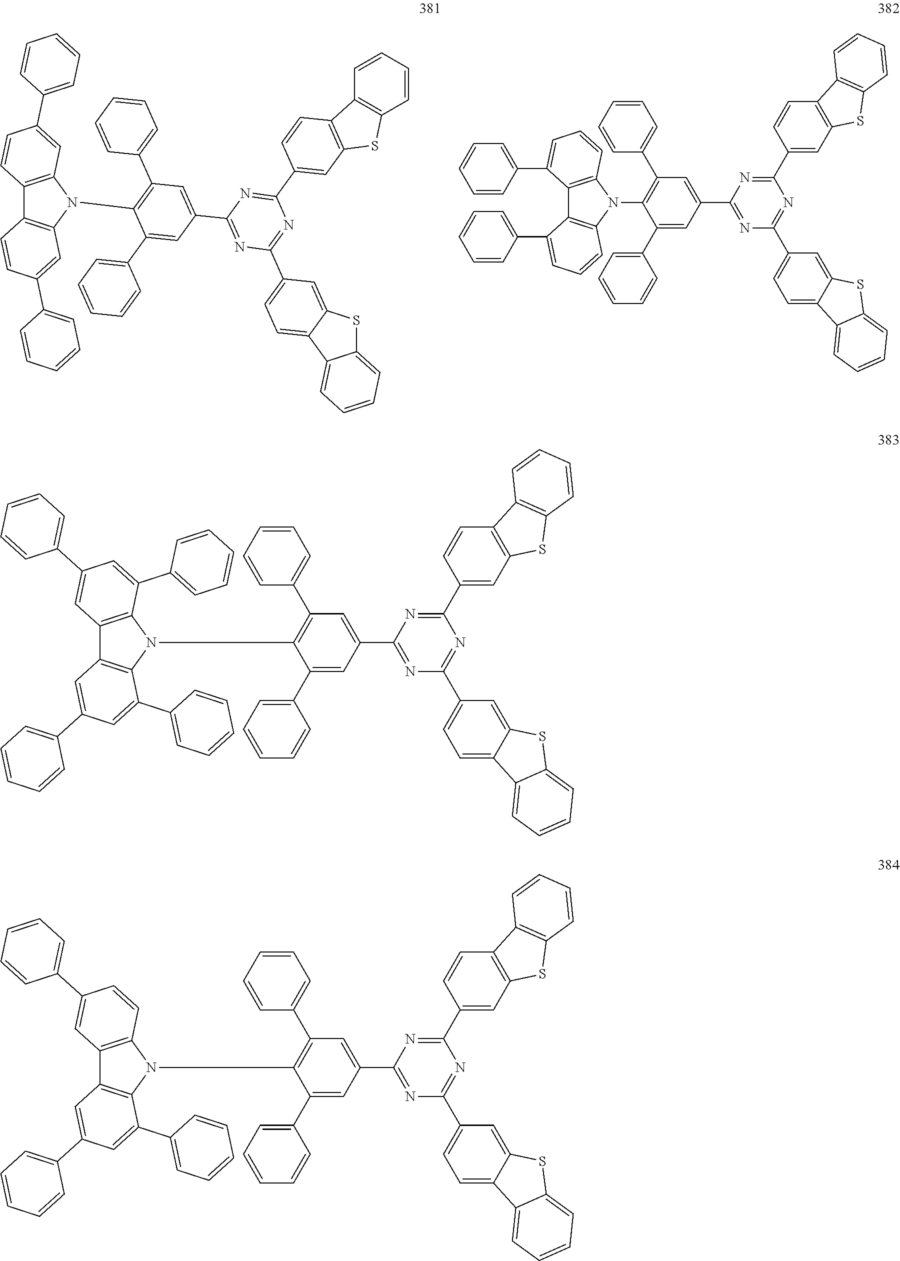

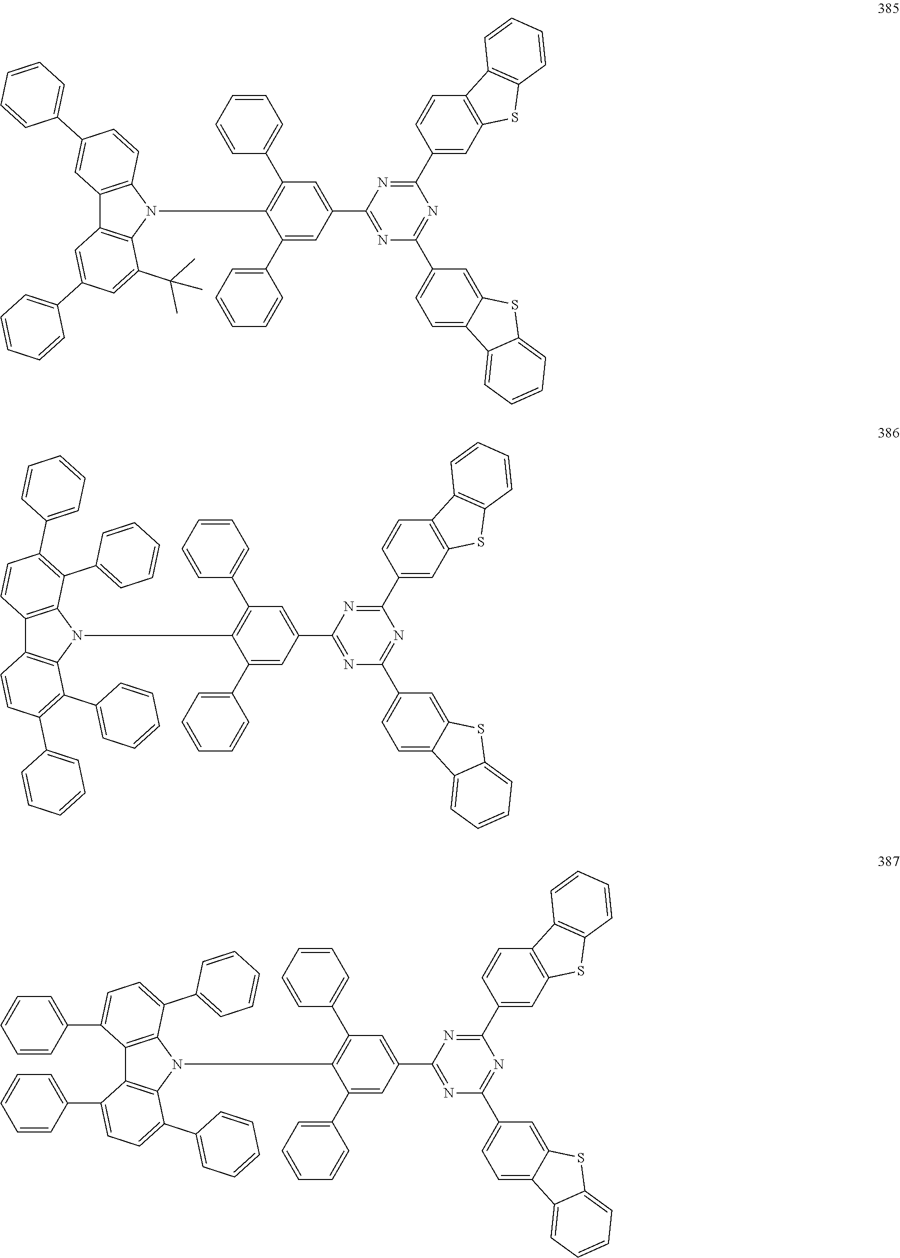 [SMILES: CC(C)(C)c1cc(-c2ccccc2)cc2c3cc(-c4ccccc4)ccc3n(-c3c(-c4ccccc4)cc(-c4nc(-c5ccc6c(c5)sc5ccccc56)nc(-c5ccc6c(c5)sc5ccccc56)n4)cc3-c3ccccc3)c12 ; c1ccc(-c2cc(-c3nc(-c4ccc5c(c4)sc4ccccc45)nc(-c4ccc5c(c4)sc4ccccc45)n3)cc(-c3ccccc3)c2-n2c3c(-c4ccccc4)c(-c4ccccc4)ccc3c3ccc(-c4ccccc4)c(-c4ccccc4)c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3c(-c4ccccc4)ccc(-c4ccccc4)c3c3c(-c4ccccc4)ccc(-c4ccccc4)c32)cc1]

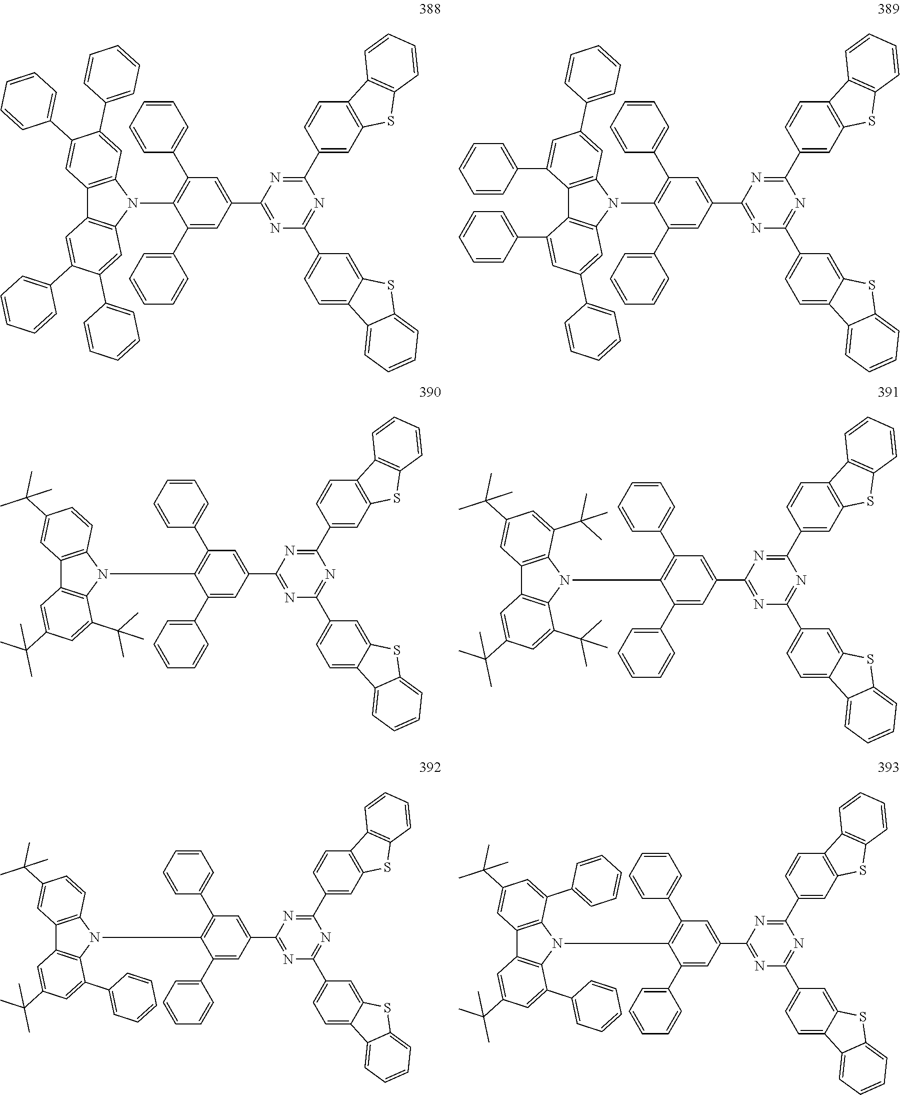

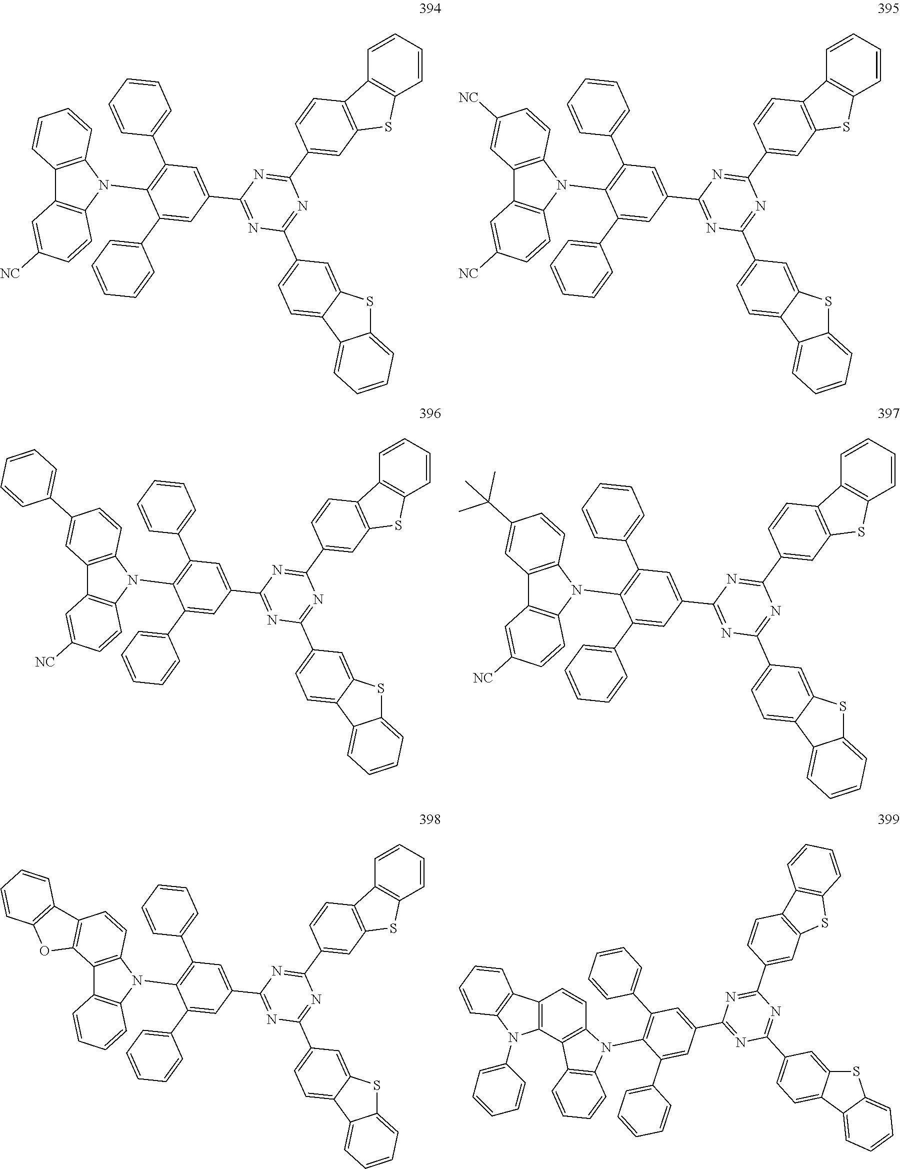

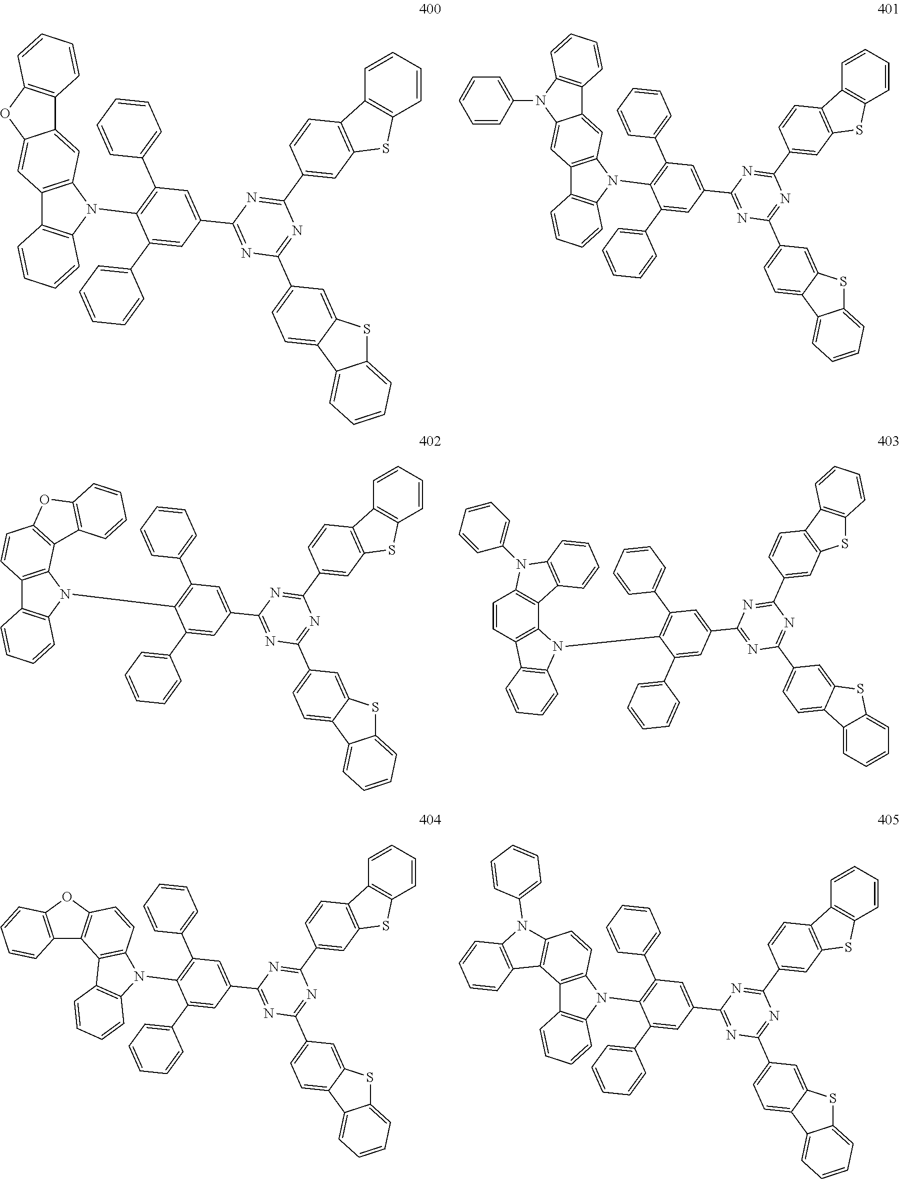

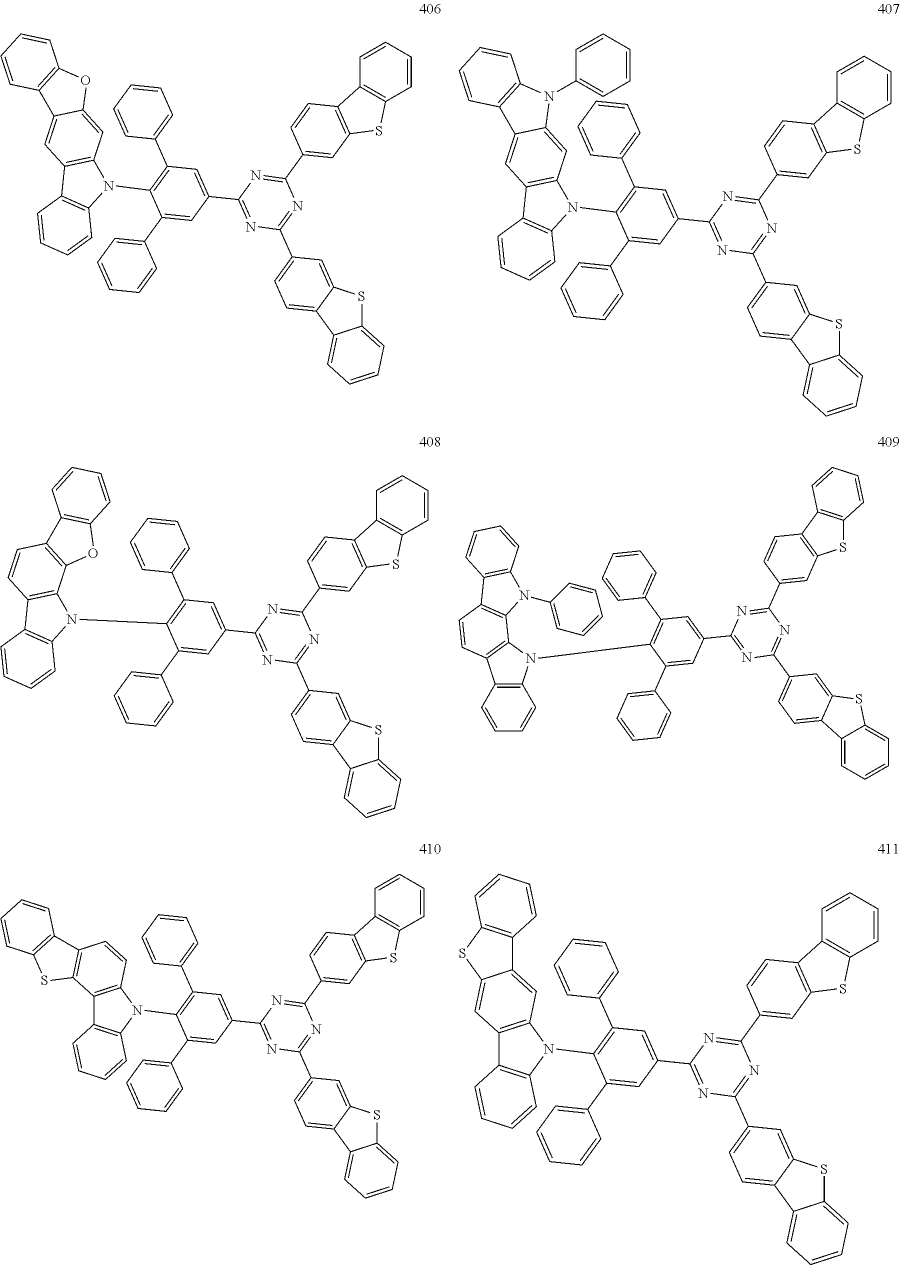

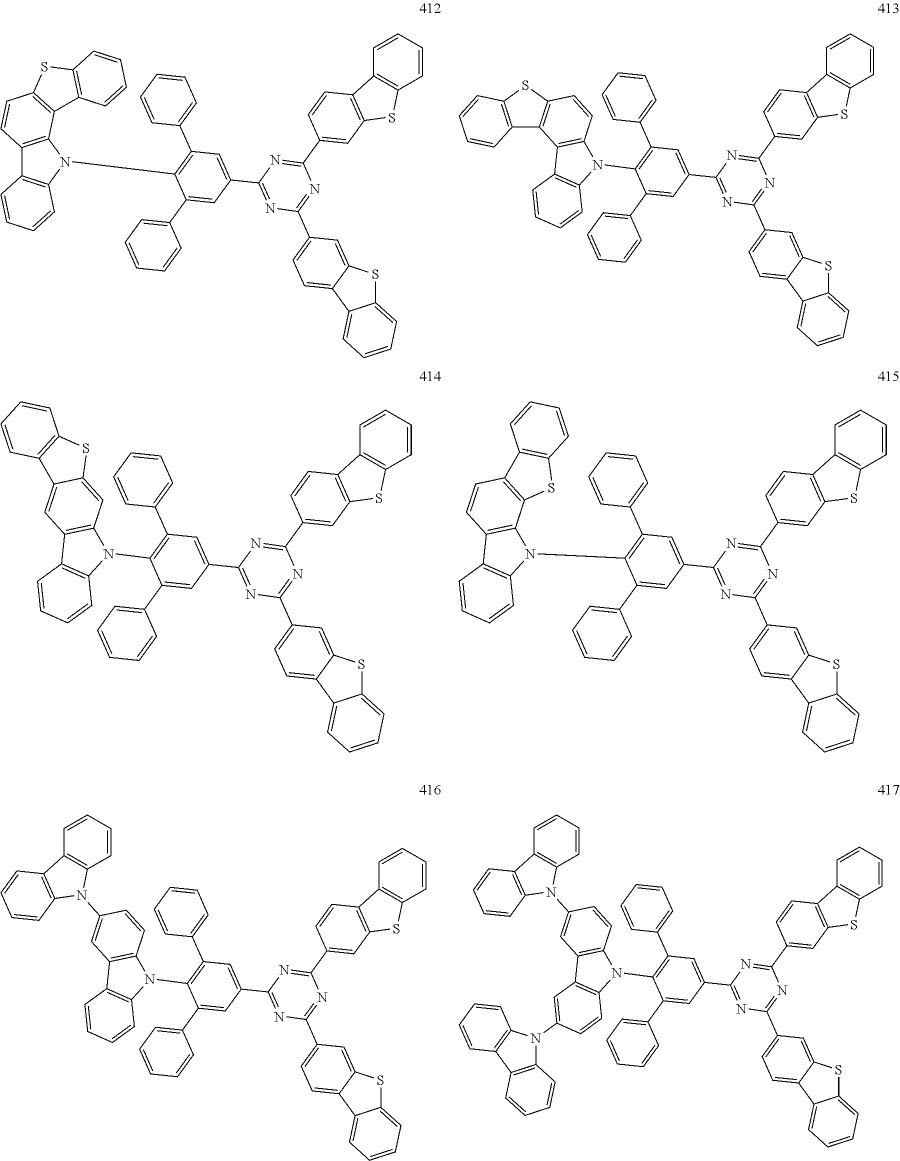

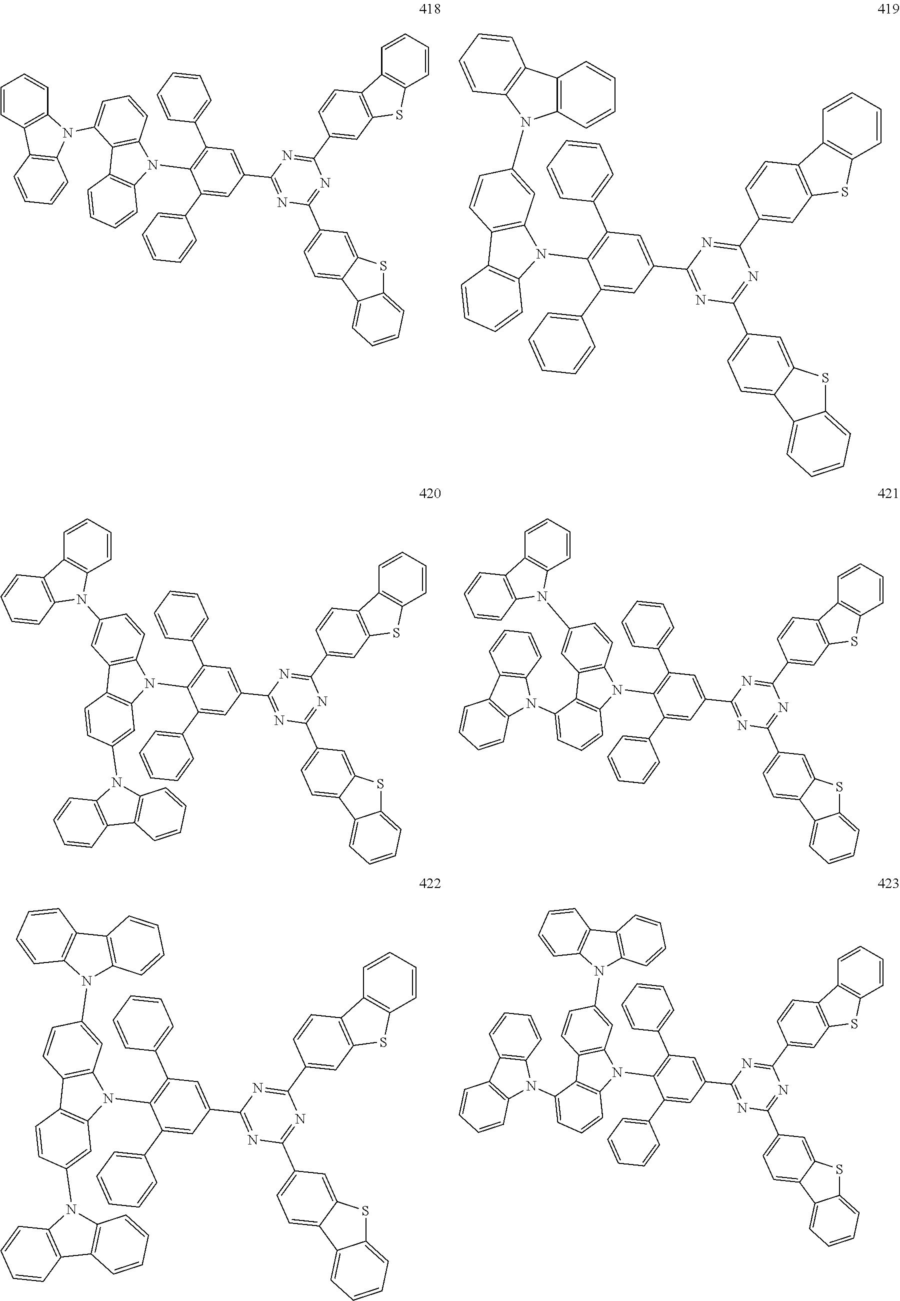

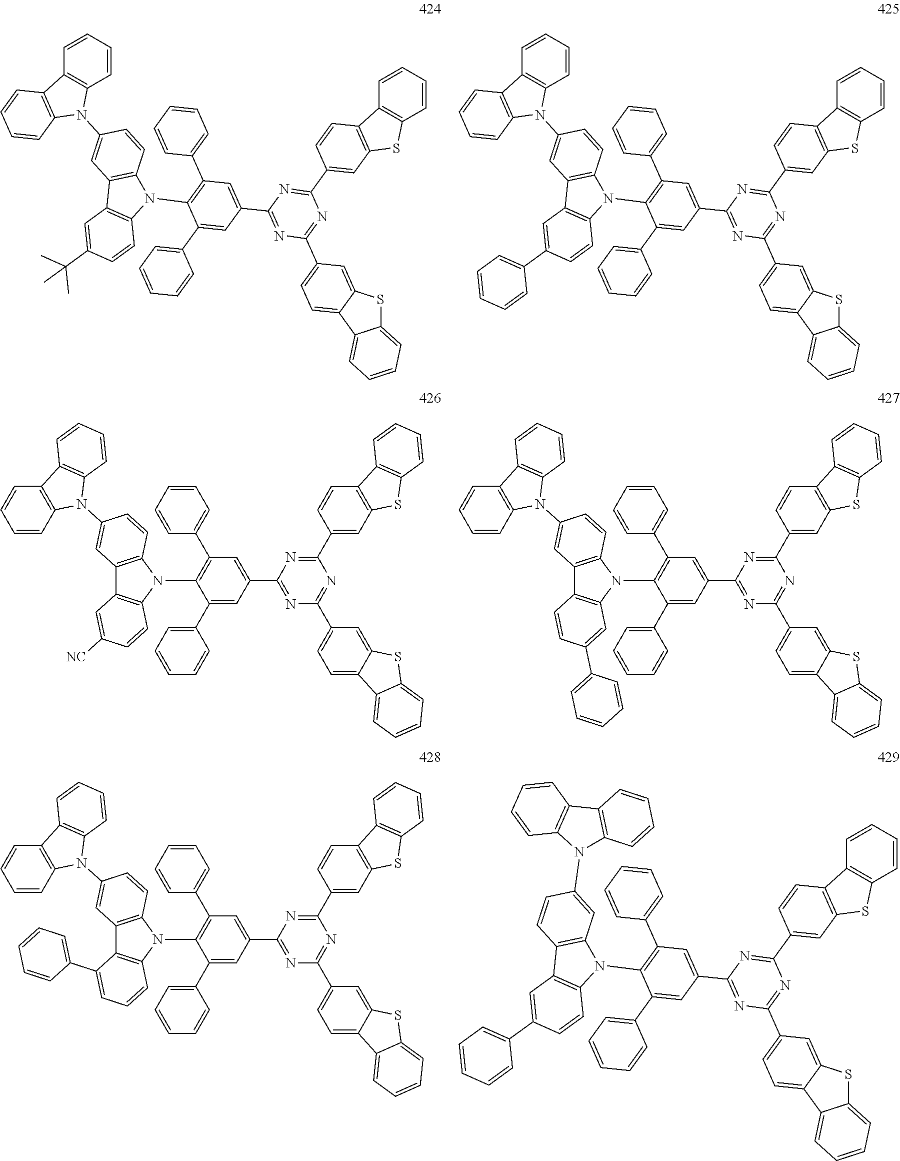

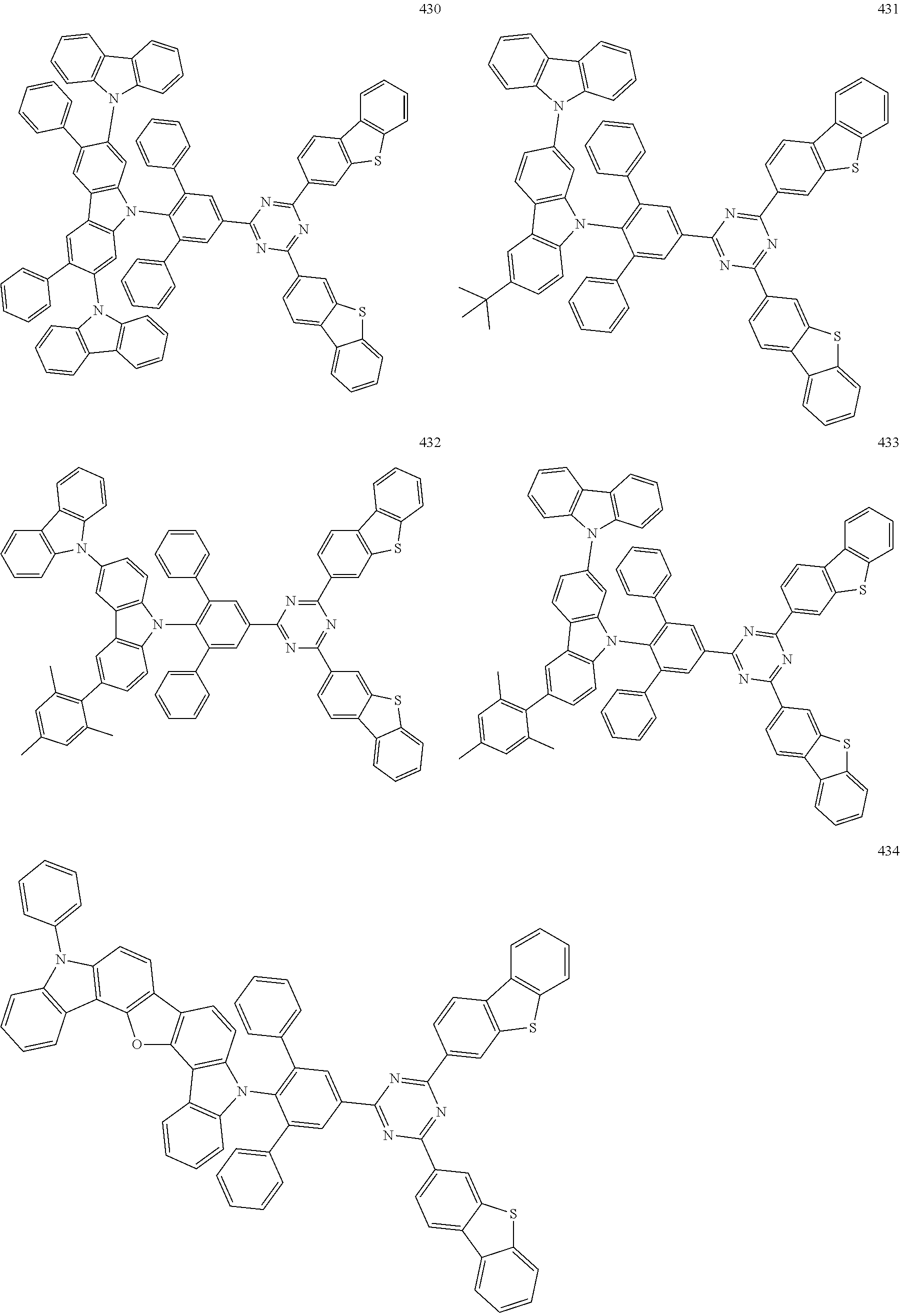

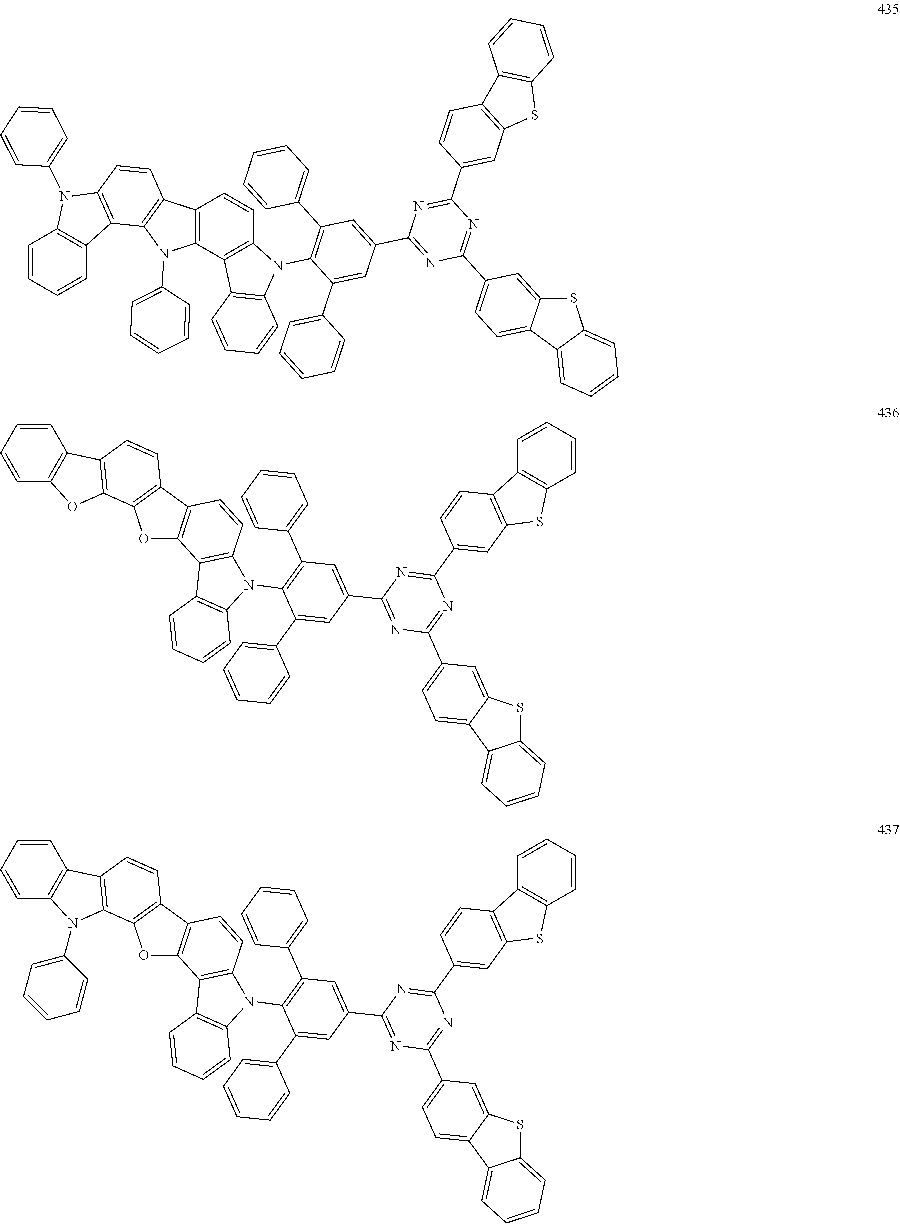

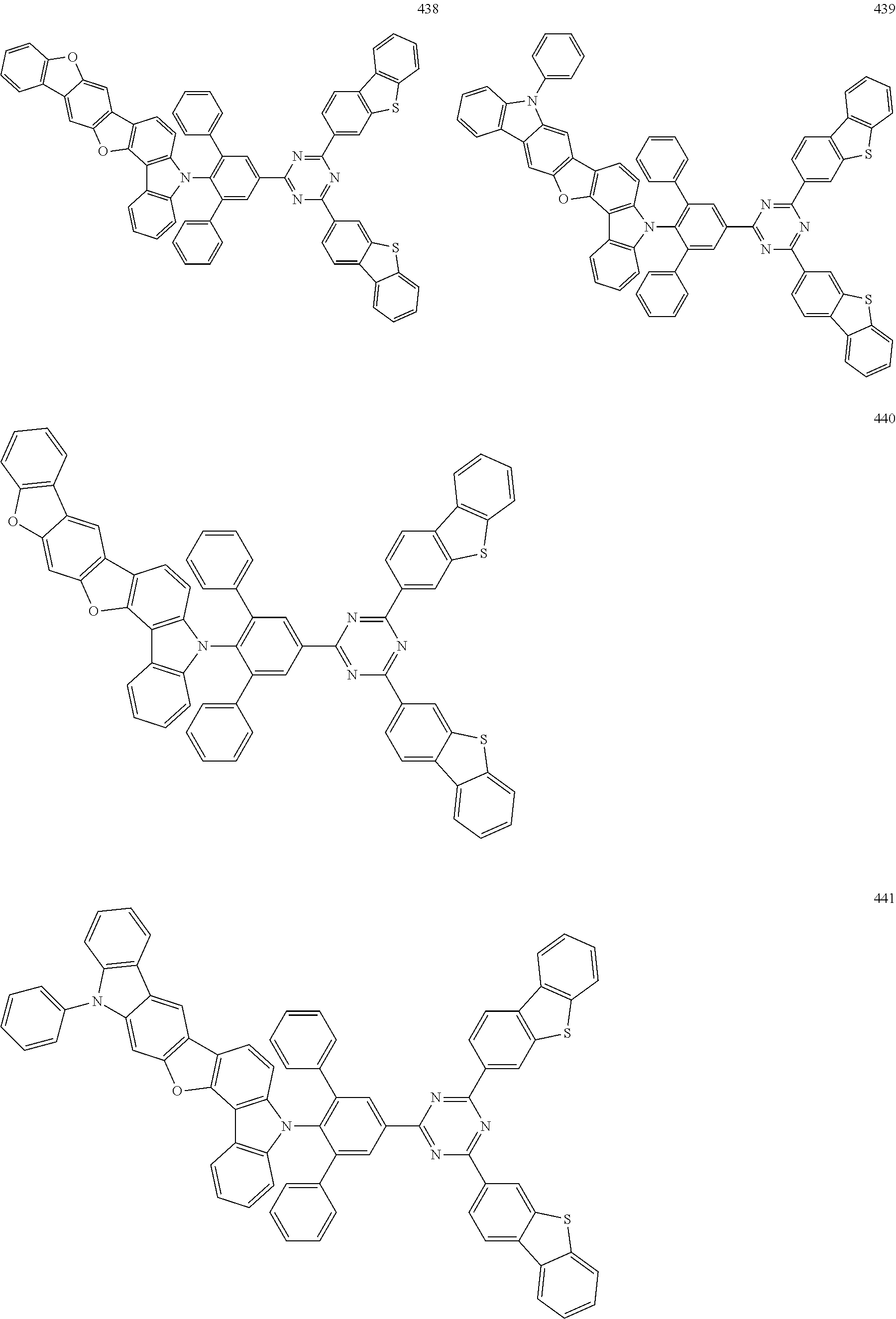

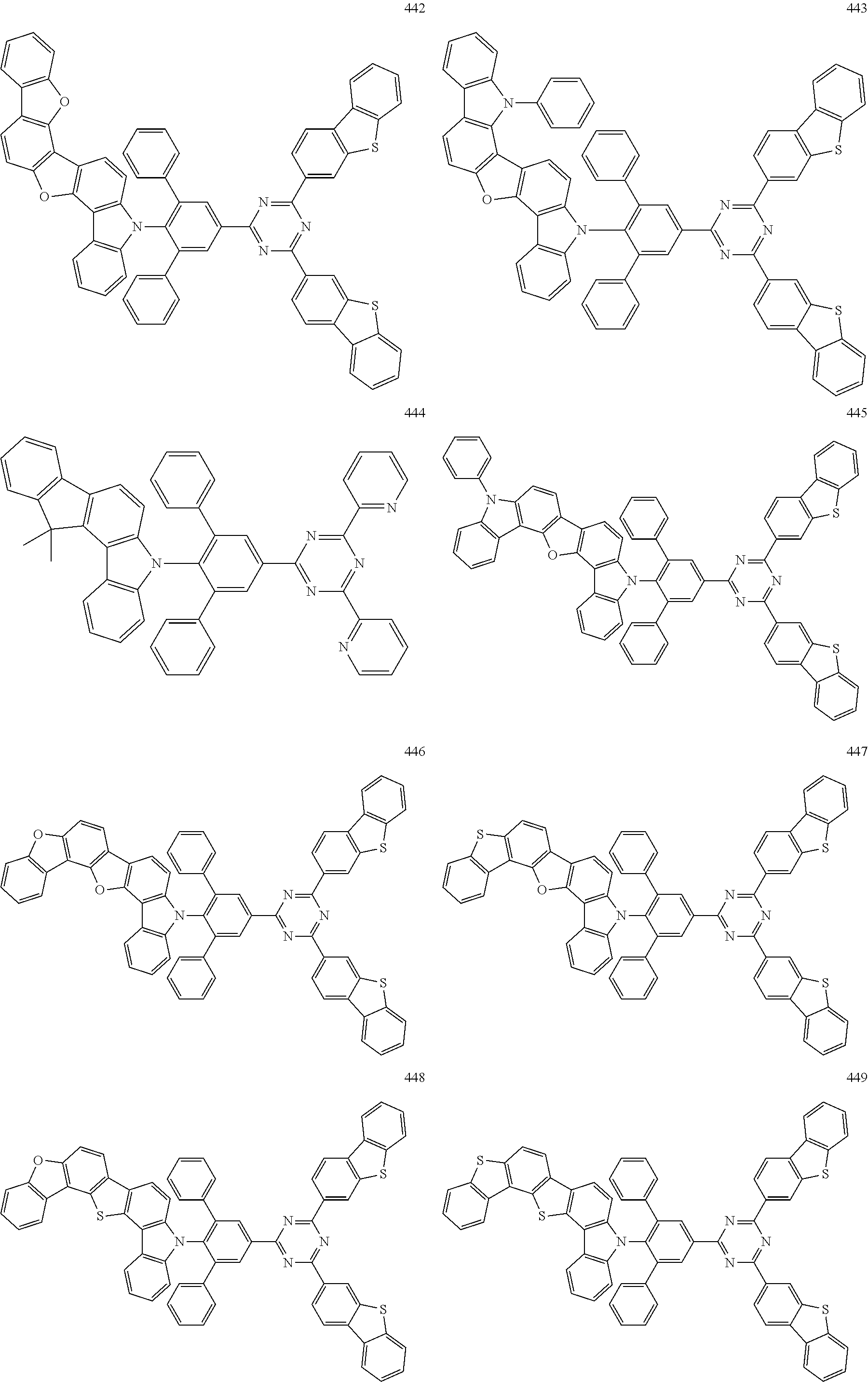

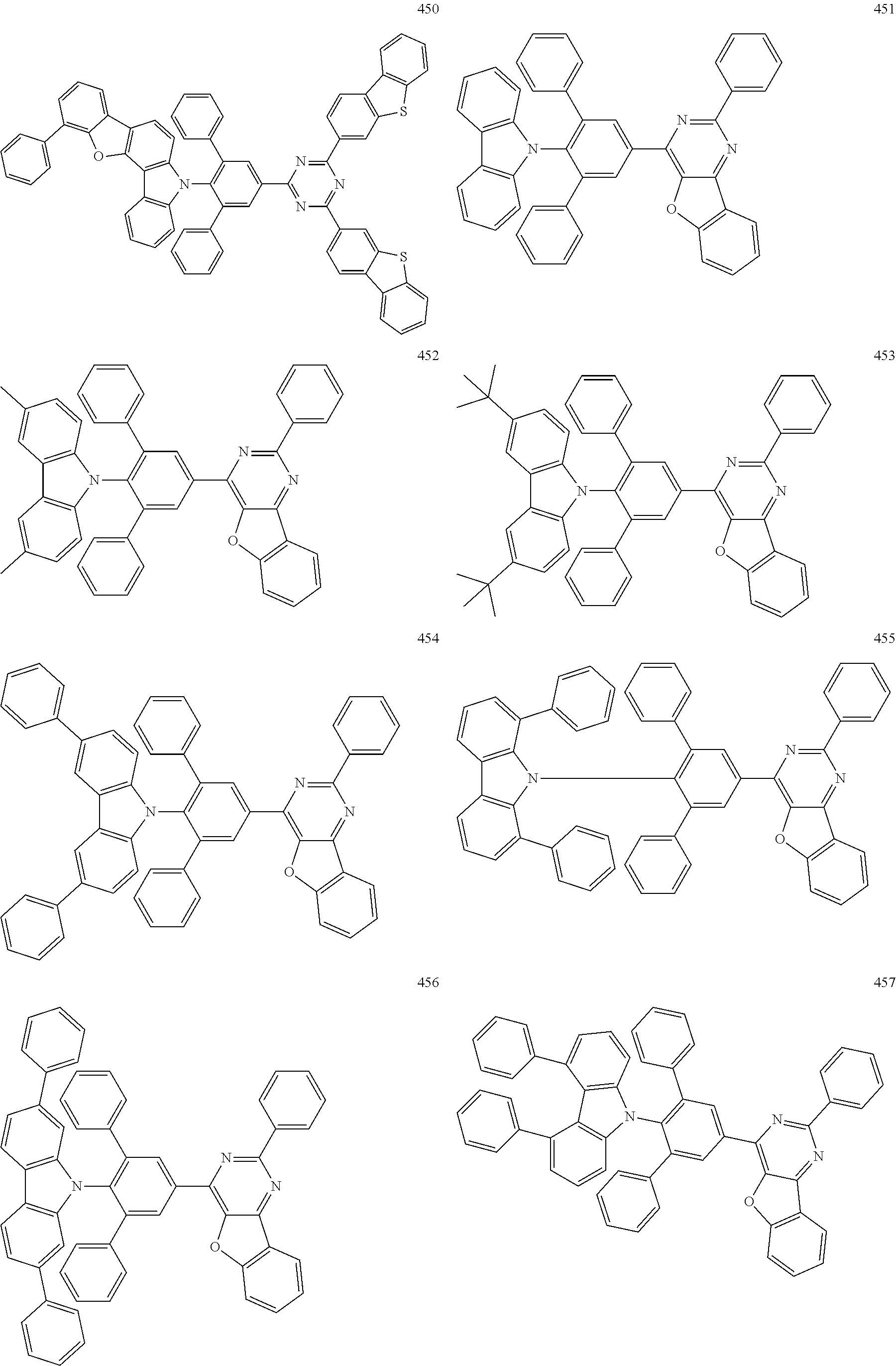

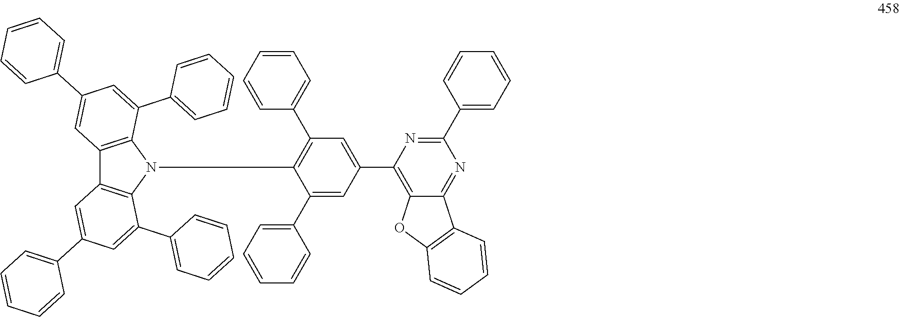 [SMILES: c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1]

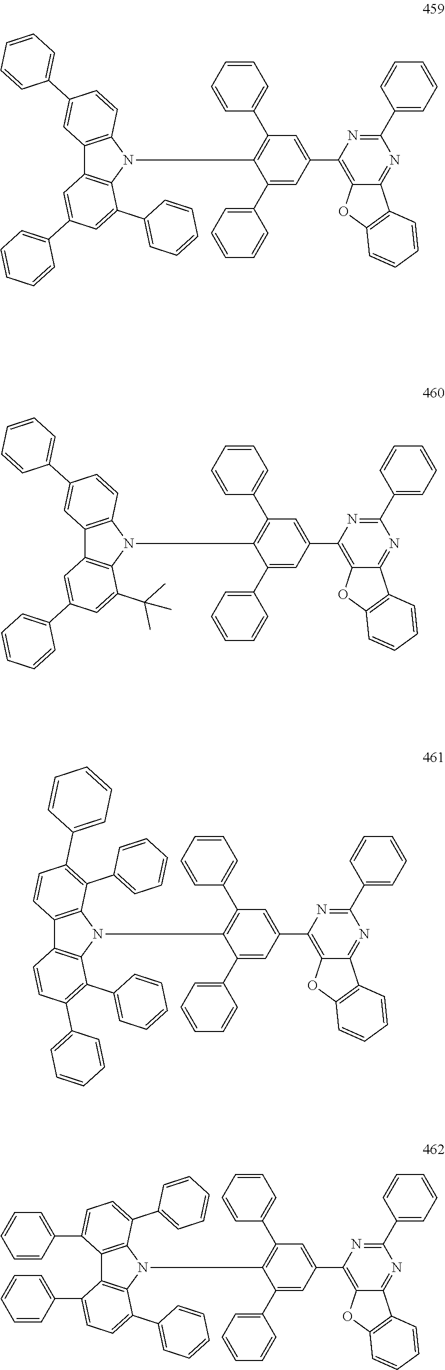 [SMILES: c1ccc(-c2ccc3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)nc5c4oc4ccccc45)cc3-c3ccccc3)c12 ; c1ccc(-c2nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)c3oc4ccccc4c3n2)cc1 ; c1ccc(-c2nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)c3oc4ccccc4c3n2)cc1]

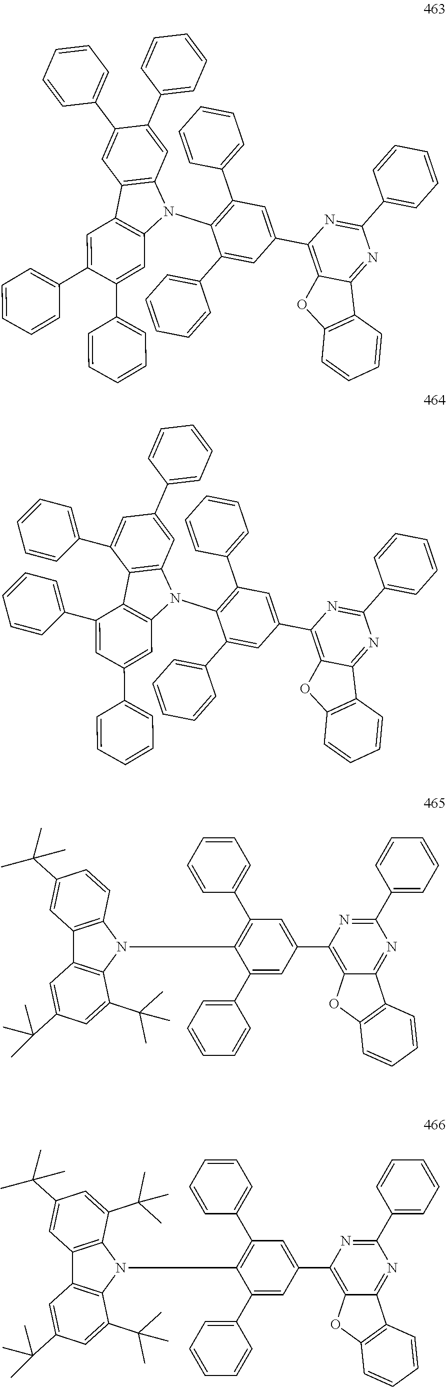 [SMILES: c1ccc(-c2nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)c3oc4ccccc4c3n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)nc6c5oc5ccccc56)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1]

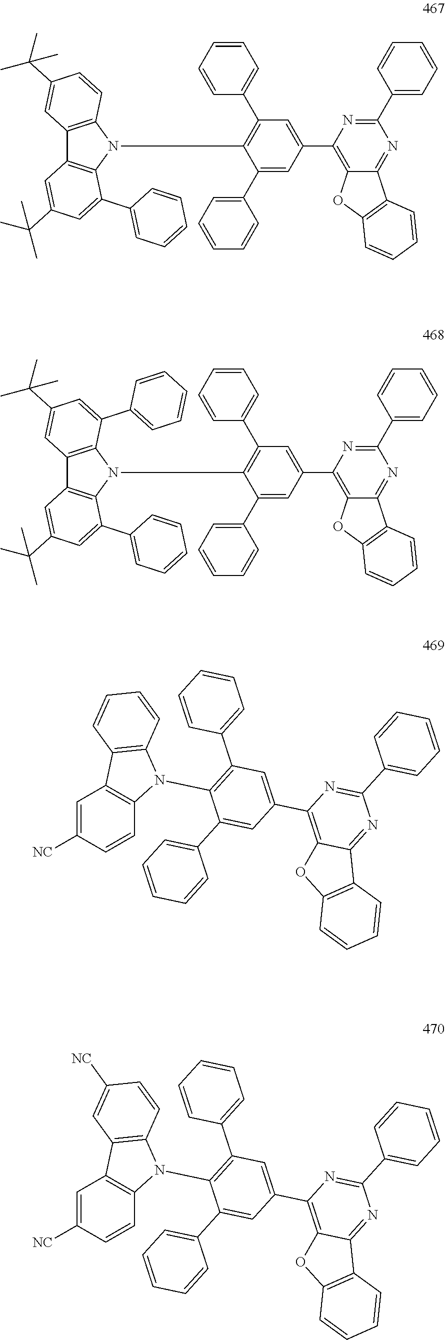 [SMILES: CC(C)(C)c1ccc2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1 ; N#Cc1ccc2c(c1)c1ccccc1n2-c1c(-c2ccccc2)cc(-c2nc(-c3ccccc3)nc3c2oc2ccccc23)cc1-c1ccccc1 ; N#Cc1ccc2c(c1)c1cc(C#N)ccc1n2-c1c(-c2ccccc2)cc(-c2nc(-c3ccccc3)nc3c2oc2ccccc23)cc1-c1ccccc1]

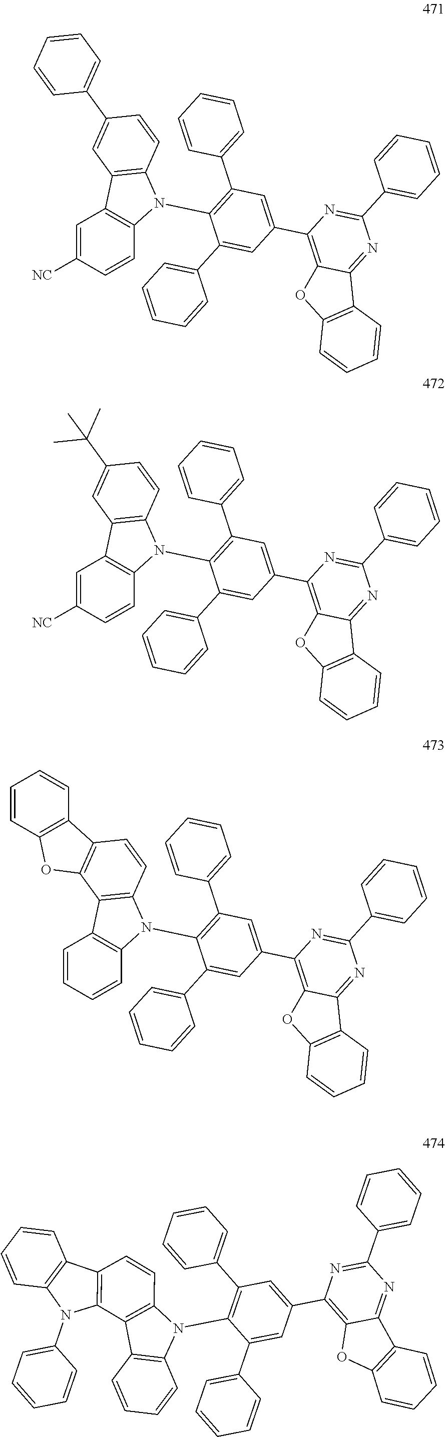 [SMILES: N#Cc1ccc2c(c1)c1cc(-c3ccccc3)ccc1n2-c1c(-c2ccccc2)cc(-c2nc(-c3ccccc3)nc3c2oc2ccccc23)cc1-c1ccccc1 ; CC(C)(C)c1ccc2c(c1)c1cc(C#N)ccc1n2-c1c(-c2ccccc2)cc(-c2nc(-c3ccccc3)nc3c2oc2ccccc23)cc1-c1ccccc1 ; c1ccc(-c2nc(-c3cc(-c4ccccc4)c(-n4c5ccccc5c5c6oc7ccccc7c6ccc54)c(-c4ccccc4)c3)c3oc4ccccc4c3n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)c3oc4ccccc4c3n2)cc1]

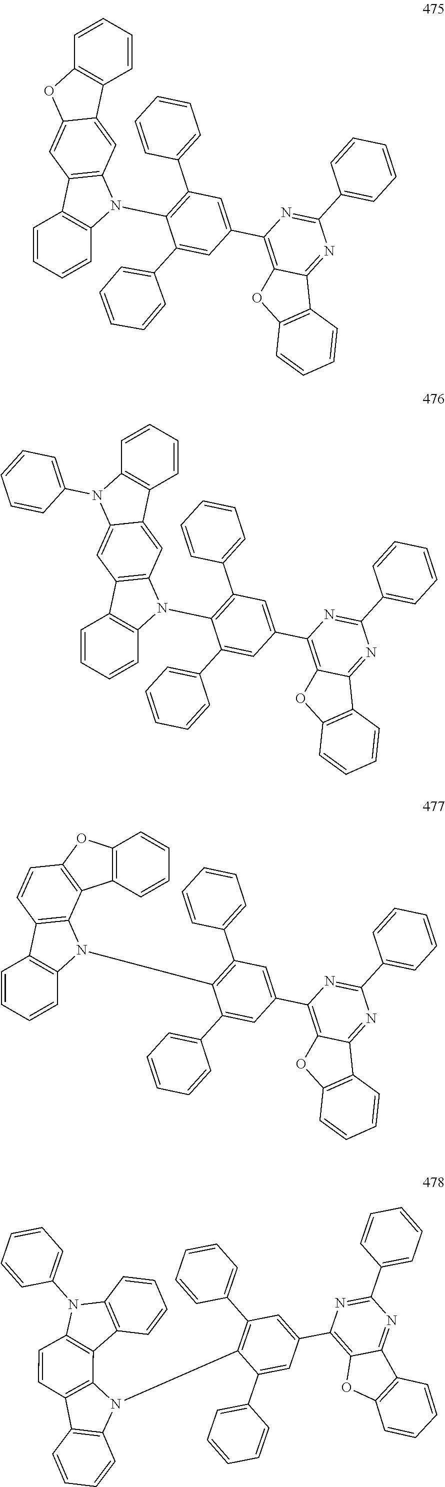 [SMILES: c1ccc(-c2nc(-c3cc(-c4ccccc4)c(-n4c5ccccc5c5cc6oc7ccccc7c6cc54)c(-c4ccccc4)c3)c3oc4ccccc4c3n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)c3oc4ccccc4c3n2)cc1 ; c1ccc(-c2nc(-c3cc(-c4ccccc4)c(-n4c5ccccc5c5ccc6oc7ccccc7c6c54)c(-c4ccccc4)c3)c3oc4ccccc4c3n2)cc1 ; c1ccc(-c2nc(-c3cc(-c4ccccc4)c(-n4c5ccccc5c5ccc6c(c7ccccc7n6-c6ccccc6)c54)c(-c4ccccc4)c3)c3oc4ccccc4c3n2)cc1]

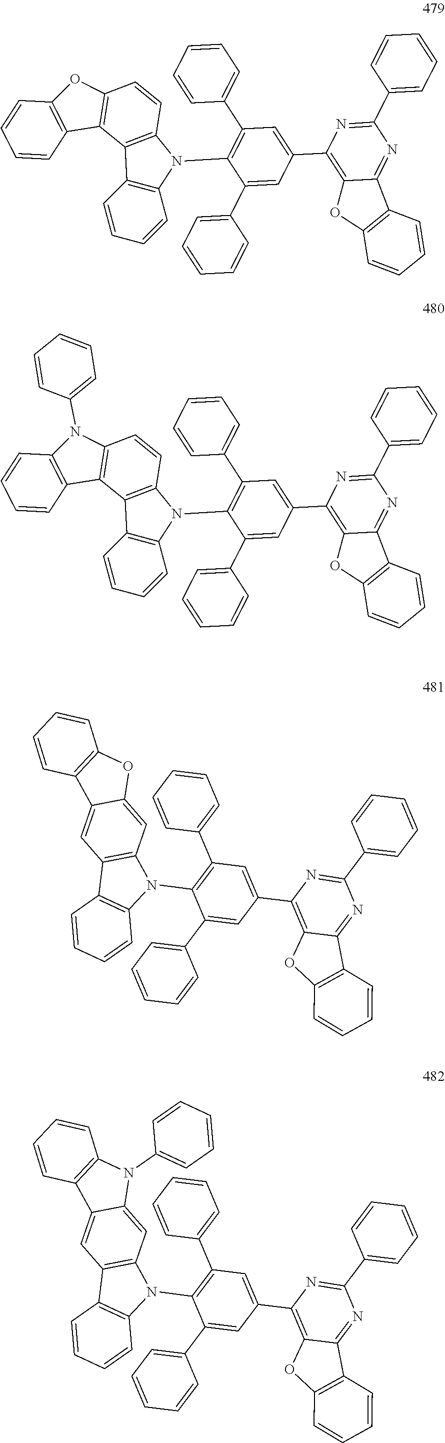 [SMILES: c1ccc(-c2nc(-c3cc(-c4ccccc4)c(-n4c5ccccc5c5c6c(ccc54)oc4ccccc46)c(-c4ccccc4)c3)c3oc4ccccc4c3n2)cc1 ; c1ccc(-c2nc(-c3cc(-c4ccccc4)c(-n4c5ccccc5c5c6c7ccccc7n(-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5cc6c(cc54)oc4ccccc46)c(-c4ccccc4)c3)c3oc4ccccc4c3n2)cc1 ; c1ccc(-c2nc(-c3cc(-c4ccccc4)c(-n4c5ccccc5c5cc6c7ccccc7n(-c7ccccc7)c6cc54)c(-c4ccccc4)c3)c3oc4ccccc4c3n2)cc1]

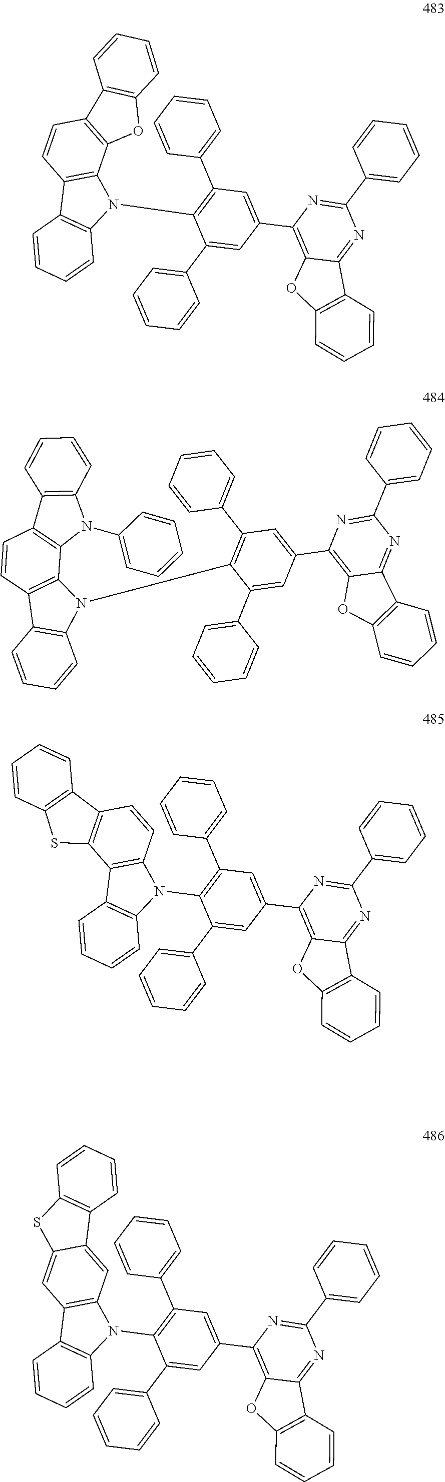 [SMILES: c1ccc(-c2nc(-c3cc(-c4ccccc4)c(-n4c5ccccc5c5ccc6c7ccccc7oc6c54)c(-c4ccccc4)c3)c3oc4ccccc4c3n2)cc1 ; c1ccc(-c2nc(-c3cc(-c4ccccc4)c(-n4c5ccccc5c5ccc6c7ccccc7n(-c7ccccc7)c6c54)c(-c4ccccc4)c3)c3oc4ccccc4c3n2)cc1 ; c1ccc(-c2nc(-c3cc(-c4ccccc4)c(-n4c5ccccc5c5c6sc7ccccc7c6ccc54)c(-c4ccccc4)c3)c3oc4ccccc4c3n2)cc1 ; c1ccc(-c2nc(-c3cc(-c4ccccc4)c(-n4c5ccccc5c5cc6sc7ccccc7c6cc54)c(-c4ccccc4)c3)c3oc4ccccc4c3n2)cc1]

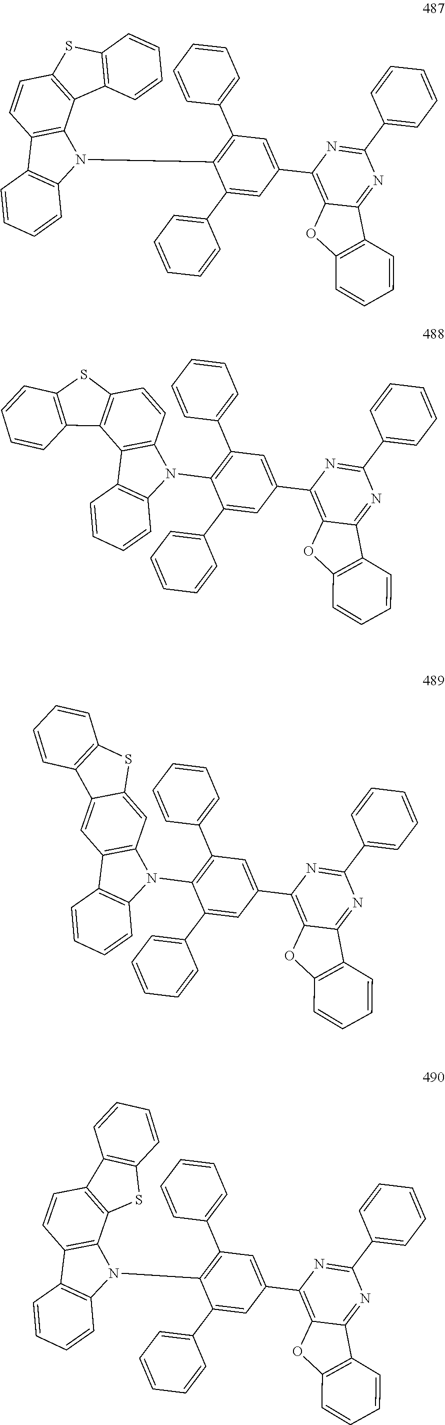 [SMILES: c1ccc(-c2nc(-c3cc(-c4ccccc4)c(-n4c5ccccc5c5ccc6sc7ccccc7c6c54)c(-c4ccccc4)c3)c3oc4ccccc4c3n2)cc1 ; c1ccc(-c2nc(-c3cc(-c4ccccc4)c(-n4c5ccccc5c5c6c(ccc54)sc4ccccc46)c(-c4ccccc4)c3)c3oc4ccccc4c3n2)cc1 ; c1ccc(-c2nc(-c3cc(-c4ccccc4)c(-n4c5ccccc5c5cc6c(cc54)sc4ccccc46)c(-c4ccccc4)c3)c3oc4ccccc4c3n2)cc1 ; c1ccc(-c2nc(-c3cc(-c4ccccc4)c(-n4c5ccccc5c5ccc6c7ccccc7sc6c54)c(-c4ccccc4)c3)c3oc4ccccc4c3n2)cc1]

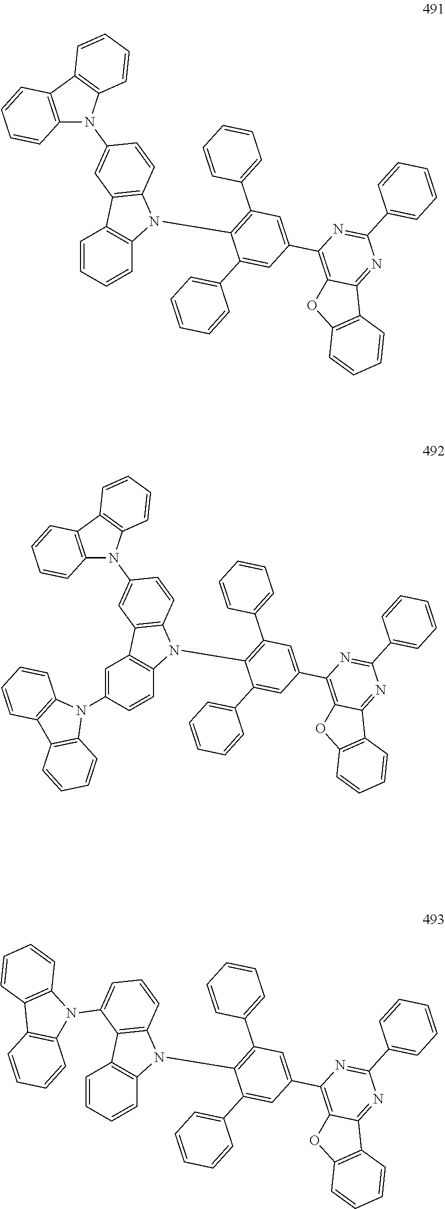 [SMILES: c1ccc(-c2nc(-c3cc(-c4ccccc4)c(-n4c5ccccc5c5cc(-n6c7ccccc7c7ccccc76)ccc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)c3oc4ccccc4c3n2)cc1 ; c1ccc(-c2nc(-c3cc(-c4ccccc4)c(-n4c5ccccc5c5c(-n6c7ccccc7c7ccccc76)cccc54)c(-c4ccccc4)c3)c3oc4ccccc4c3n2)cc1]

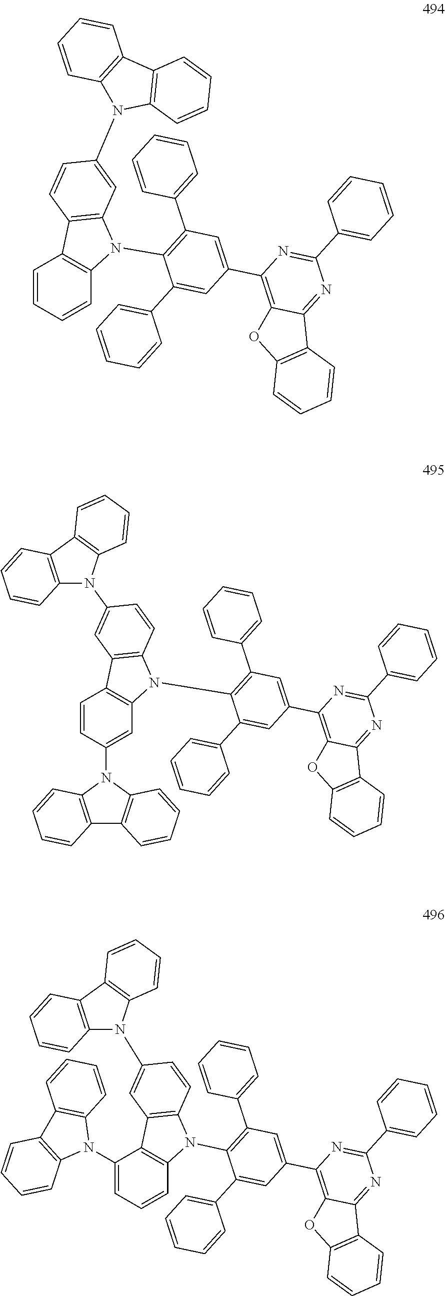 [SMILES: c1ccc(-c2nc(-c3cc(-c4ccccc4)c(-n4c5ccccc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3oc4ccccc4c3n2)cc1]

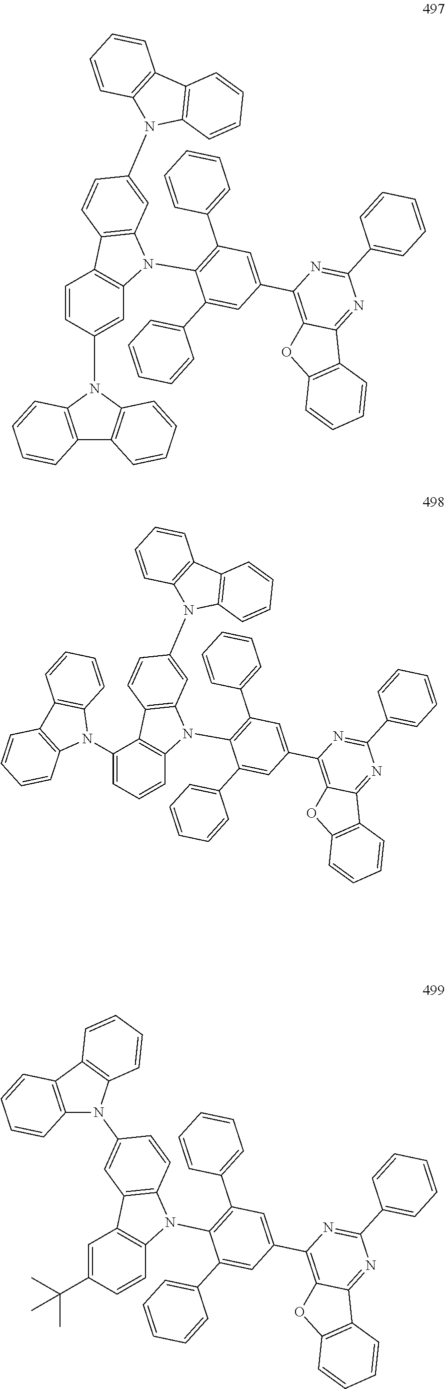 [SMILES: c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)c3oc4ccccc4c3n2)cc1 ; CC(C)(C)c1ccc2c(c1)c1cc(-n3c4ccccc4c4ccccc43)ccc1n2-c1c(-c2ccccc2)cc(-c2nc(-c3ccccc3)nc3c2oc2ccccc23)cc1-c1ccccc1]

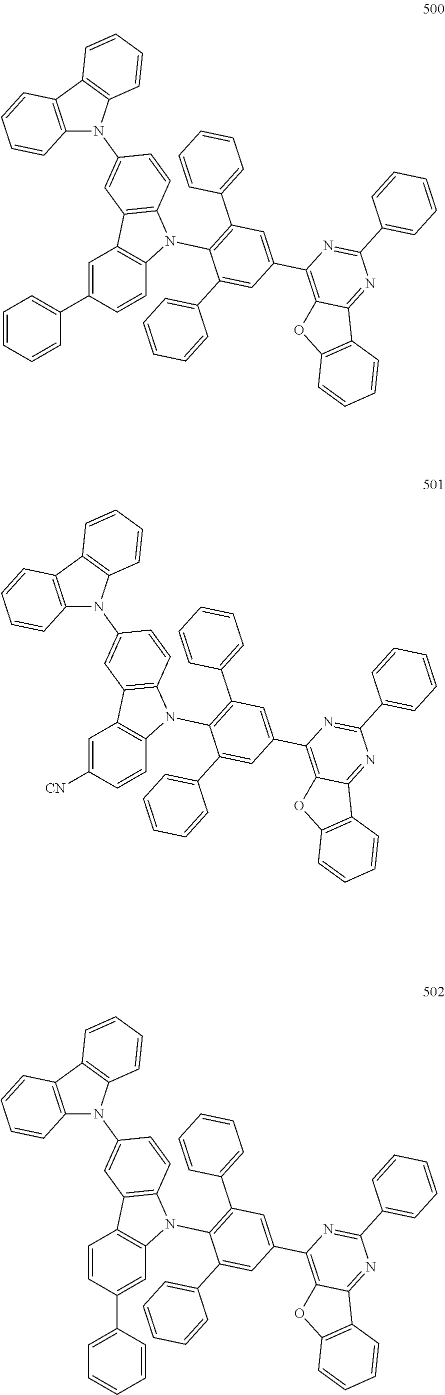

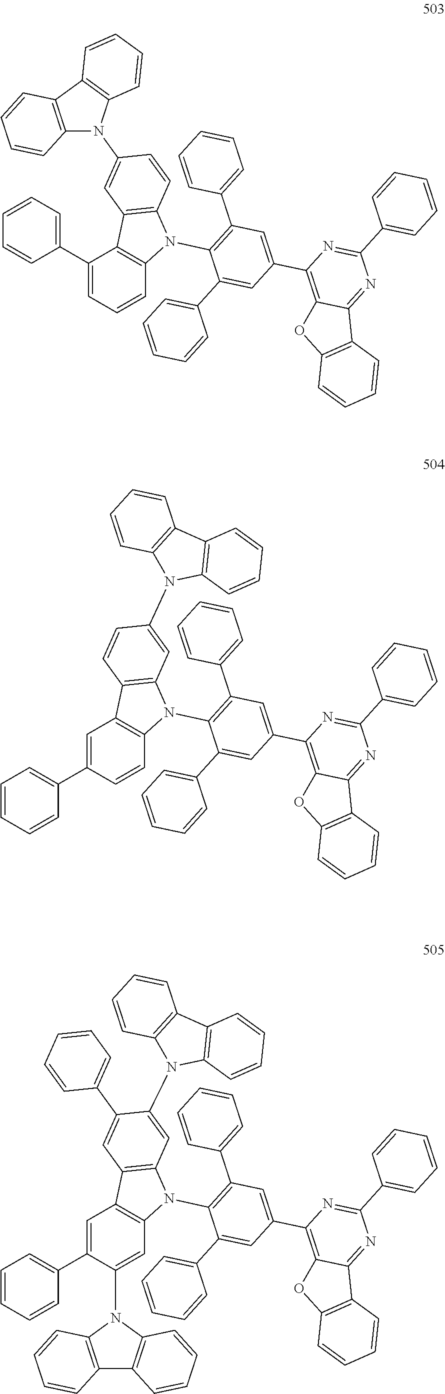 [SMILES: c1ccc(-c2nc(-c3cc(-c4ccccc4)c(-n4c5ccc(-n6c7ccccc7c7ccccc76)cc5c5c(-c6ccccc6)cccc54)c(-c4ccccc4)c3)c3oc4ccccc4c3n2)cc1 ; c1ccc(-c2ccc3c(c2)c2ccc(-n4c5ccccc5c5ccccc54)cc2n3-c2c(-c3ccccc3)cc(-c3nc(-c4ccccc4)nc4c3oc3ccccc34)cc2-c2ccccc2)cc1 ; c1ccc(-c2nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)c3oc4ccccc4c3n2)cc1]

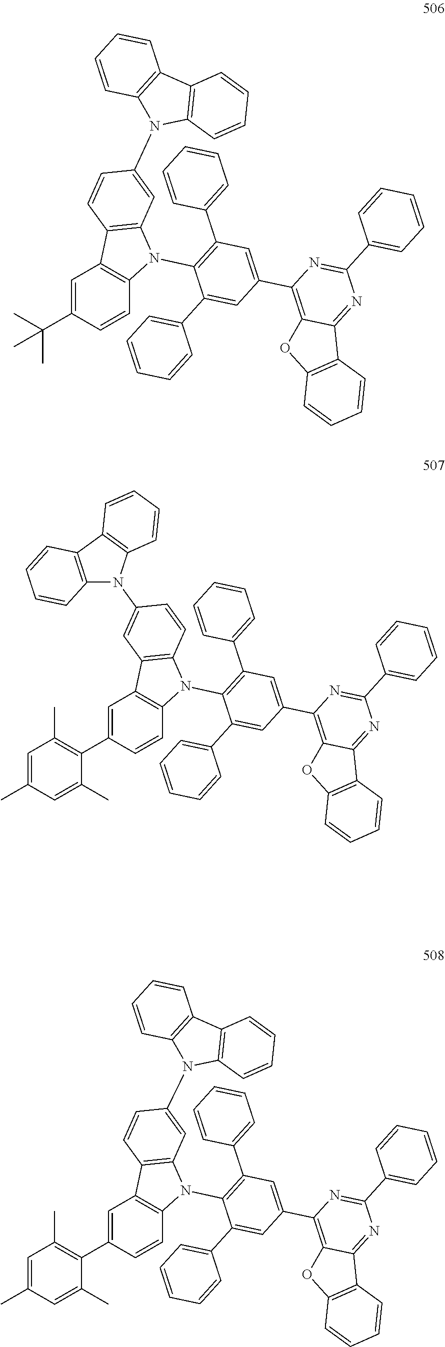 [SMILES: CC(C)(C)c1ccc2c(c1)c1ccc(-n3c4ccccc4c4ccccc43)cc1n2-c1c(-c2ccccc2)cc(-c2nc(-c3ccccc3)nc3c2oc2ccccc23)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)nc4c3oc3ccccc34)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccccc4)nc4c3oc3ccccc34)cc2-c2ccccc2)c(C)c1]

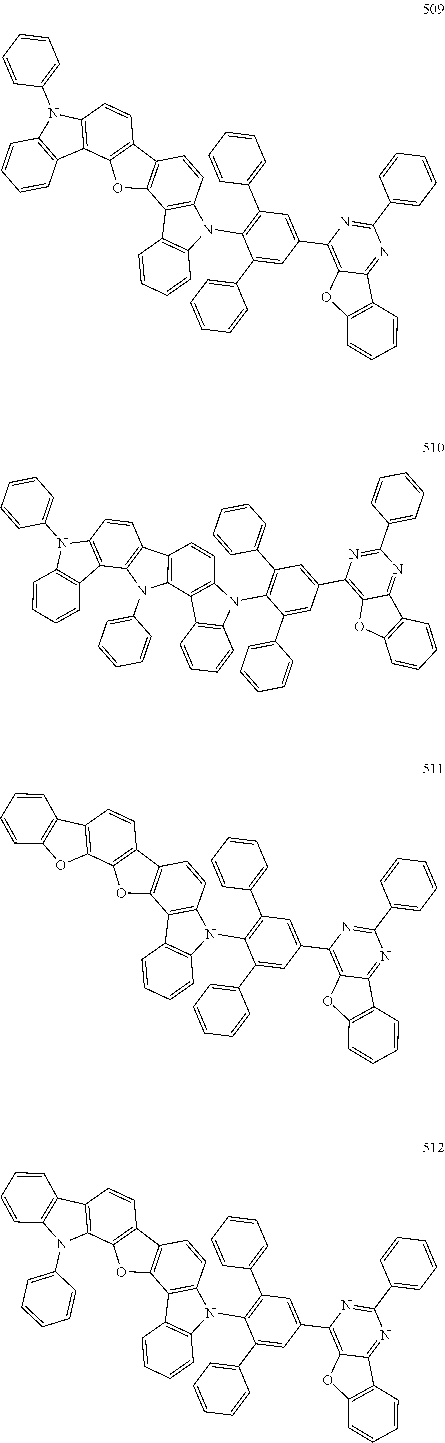 [SMILES: c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)c3oc4ccccc4c3n2)cc1 ; c1ccc(-c2nc(-c3cc(-c4ccccc4)c(-n4c5ccccc5c5c6oc7c(ccc8c9ccccc9oc87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8c9ccccc9n(-c9ccccc9)c87)c6ccc54)c(-c4ccccc4)c3)c3oc4ccccc4c3n2)cc1]

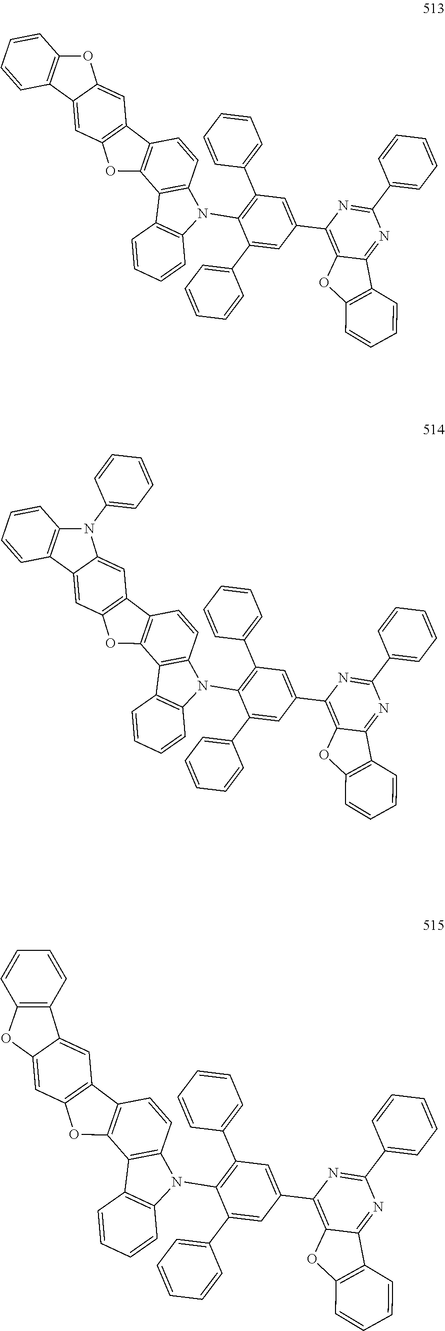 [SMILES: c1ccc(-c2nc(-c3cc(-c4ccccc4)c(-n4c5ccccc5c5c6oc7cc8c(cc7c6ccc54)oc4ccccc48)c(-c4ccccc4)c3)c3oc4ccccc4c3n2)cc1 ; c1ccc(-c2nc(-c3cc(-c4ccccc4)c(-n4c5ccccc5c5c6oc7cc8c9ccccc9n(-c9ccccc9)c8cc7c6ccc54)c(-c4ccccc4)c3)c3oc4ccccc4c3n2)cc1 ; c1ccc(-c2nc(-c3cc(-c4ccccc4)c(-n4c5ccccc5c5c6oc7cc8oc9ccccc9c8cc7c6ccc54)c(-c4ccccc4)c3)c3oc4ccccc4c3n2)cc1]

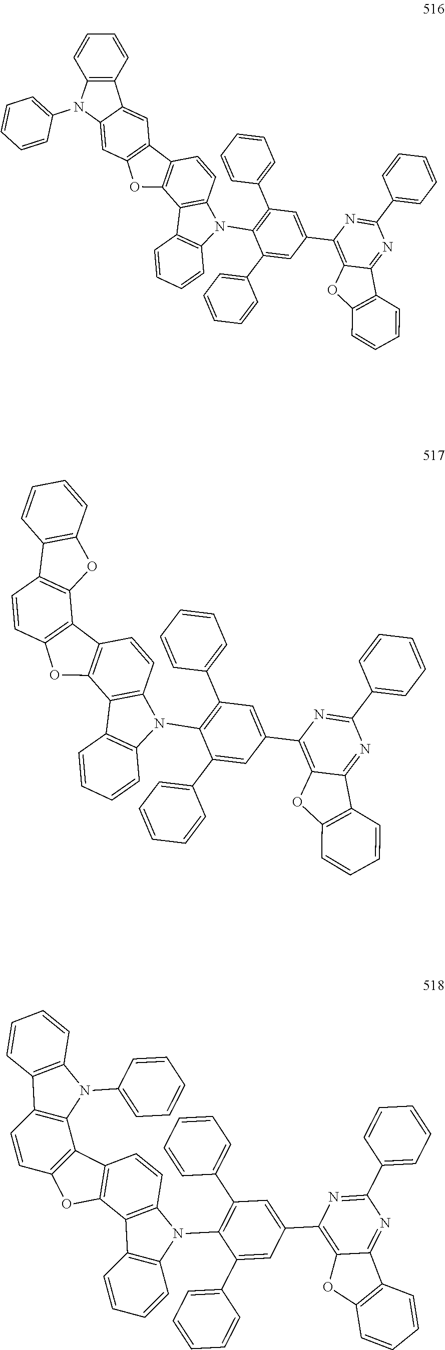 [SMILES: c1ccc(-c2nc(-c3cc(-c4ccccc4)c(-n4c5ccccc5c5c6oc7cc8c(cc7c6ccc54)c4ccccc4n8-c4ccccc4)c(-c4ccccc4)c3)c3oc4ccccc4c3n2)cc1 ; c1ccc(-c2nc(-c3cc(-c4ccccc4)c(-n4c5ccccc5c5c6oc7ccc8c9ccccc9oc8c7c6ccc54)c(-c4ccccc4)c3)c3oc4ccccc4c3n2)cc1 ; c1ccc(-c2nc(-c3cc(-c4ccccc4)c(-n4c5ccccc5c5c6oc7ccc8c9ccccc9n(-c9ccccc9)c8c7c6ccc54)c(-c4ccccc4)c3)c3oc4ccccc4c3n2)cc1]

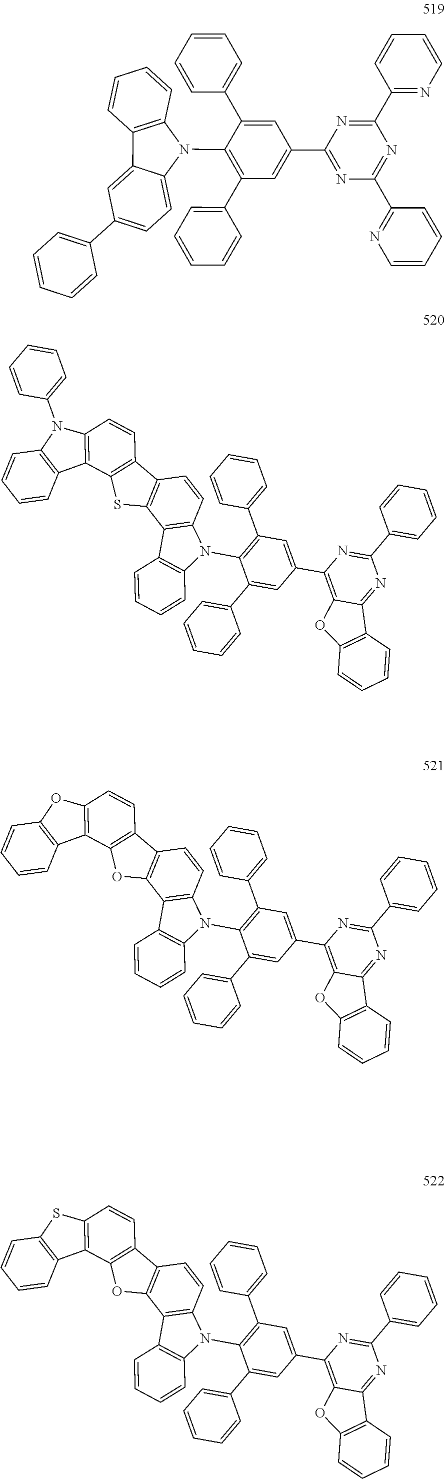 [SMILES: c1ccc(-c2ccc3c(c2)c2ccccc2n3-c2c(-c3ccccc3)cc(-c3nc(-c4ccccn4)nc(-c4ccccn4)n3)cc2-c2ccccc2)cc1 ; c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8oc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8sc9ccccc9c87)c6ccc54)c(-c4ccccc4)c3)c3oc4ccccc4c3n2)cc1]

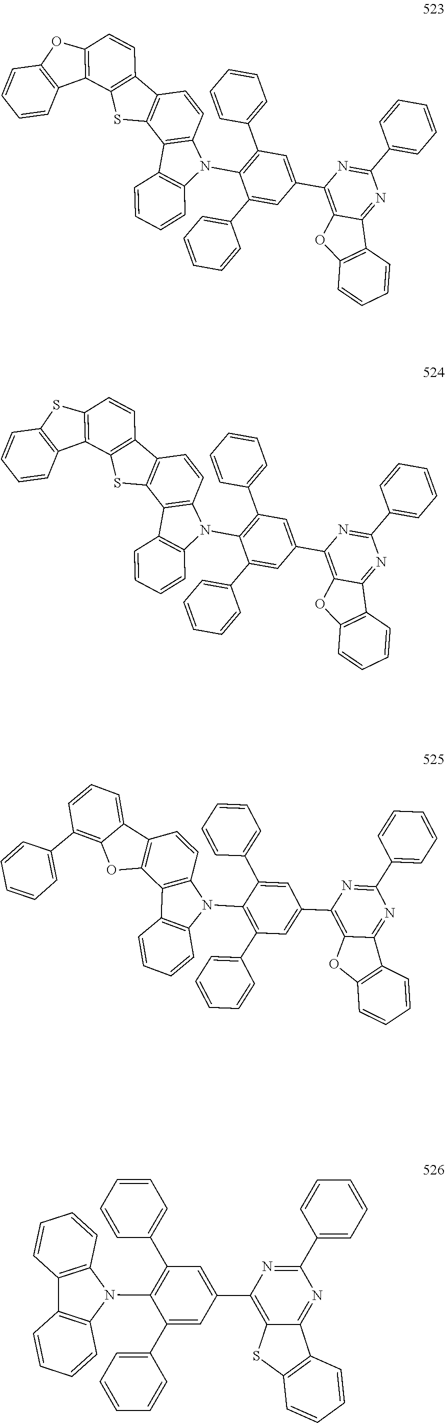 [SMILES: c1ccc(-c2nc(-c3cc(-c4ccccc4)c(-n4c5ccccc5c5c6sc7c(ccc8oc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6sc7c(ccc8sc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(-c8ccccc8)cccc7c6ccc54)c(-c4ccccc4)c3)c3oc4ccccc4c3n2)cc1 ; c1ccc(-c2nc(-c3cc(-c4ccccc4)c(-n4c5ccccc5c5ccccc54)c(-c4ccccc4)c3)c3sc4ccccc4c3n2)cc1]

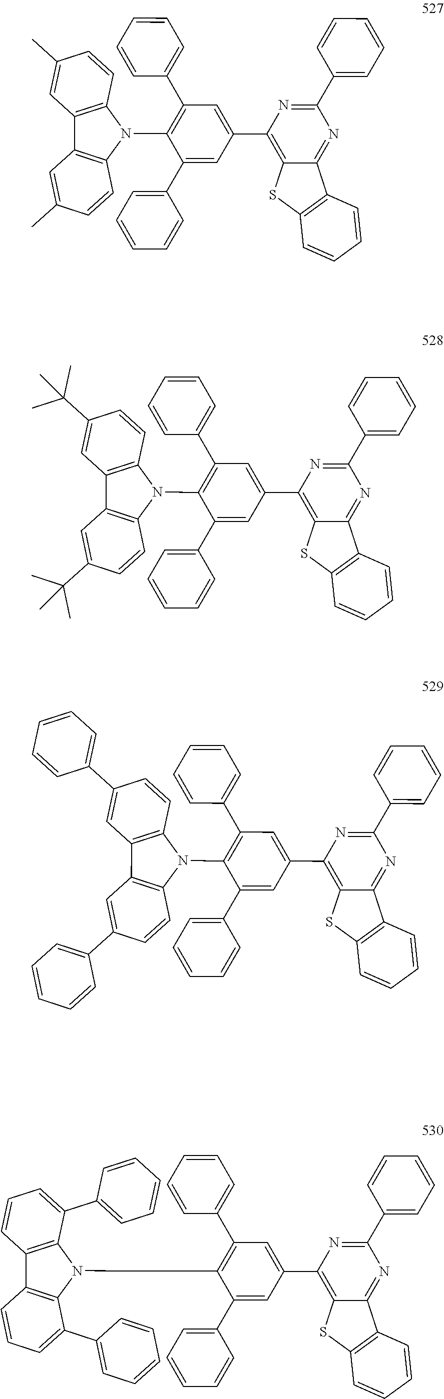 [SMILES: Cc1ccc2c(c1)c1cc(C)ccc1n2-c1c(-c2ccccc2)cc(-c2nc(-c3ccccc3)nc3c2sc2ccccc23)cc1-c1ccccc1 ; CC(C)(C)c1ccc2c(c1)c1cc(C(C)(C)C)ccc1n2-c1c(-c2ccccc2)cc(-c2nc(-c3ccccc3)nc3c2sc2ccccc23)cc1-c1ccccc1 ; c1ccc(-c2ccc3c(c2)c2cc(-c4ccccc4)ccc2n3-c2c(-c3ccccc3)cc(-c3nc(-c4ccccc4)nc4c3sc3ccccc34)cc2-c2ccccc2)cc1 ; c1ccc(-c2nc(-c3cc(-c4ccccc4)c(-n4c5c(-c6ccccc6)cccc5c5cccc(-c6ccccc6)c54)c(-c4ccccc4)c3)c3sc4ccccc4c3n2)cc1]

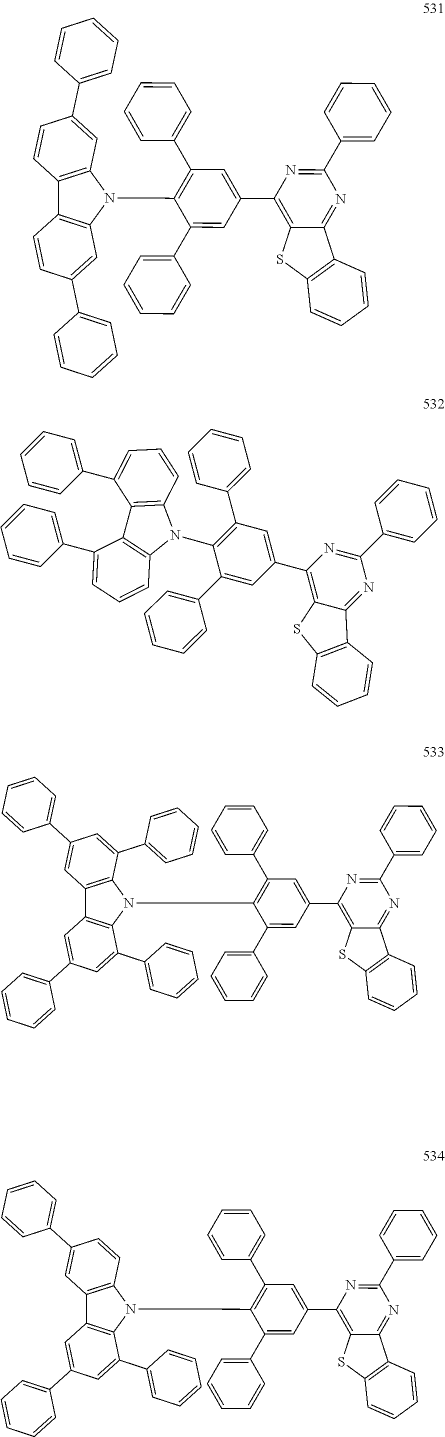 [SMILES: c1ccc(-c2ccc3c4ccc(-c5ccccc5)cc4n(-c4c(-c5ccccc5)cc(-c5nc(-c6ccccc6)nc6c5sc5ccccc56)cc4-c4ccccc4)c3c2)cc1 ; c1ccc(-c2nc(-c3cc(-c4ccccc4)c(-n4c5cccc(-c6ccccc6)c5c5c(-c6ccccc6)cccc54)c(-c4ccccc4)c3)c3sc4ccccc4c3n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc4c3sc3ccccc34)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc4c3sc3ccccc34)cc2-c2ccccc2)cc1]

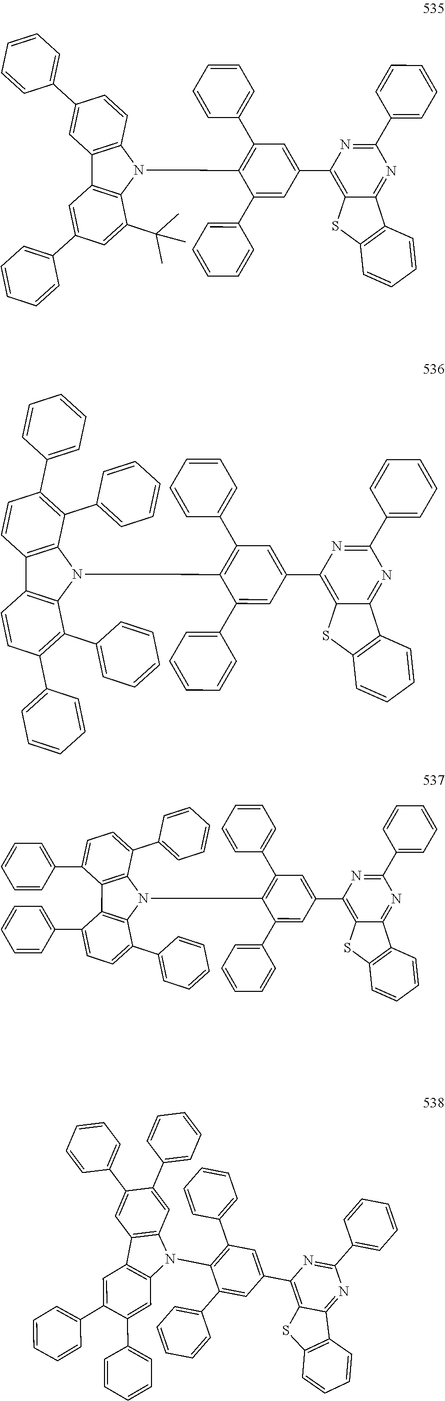

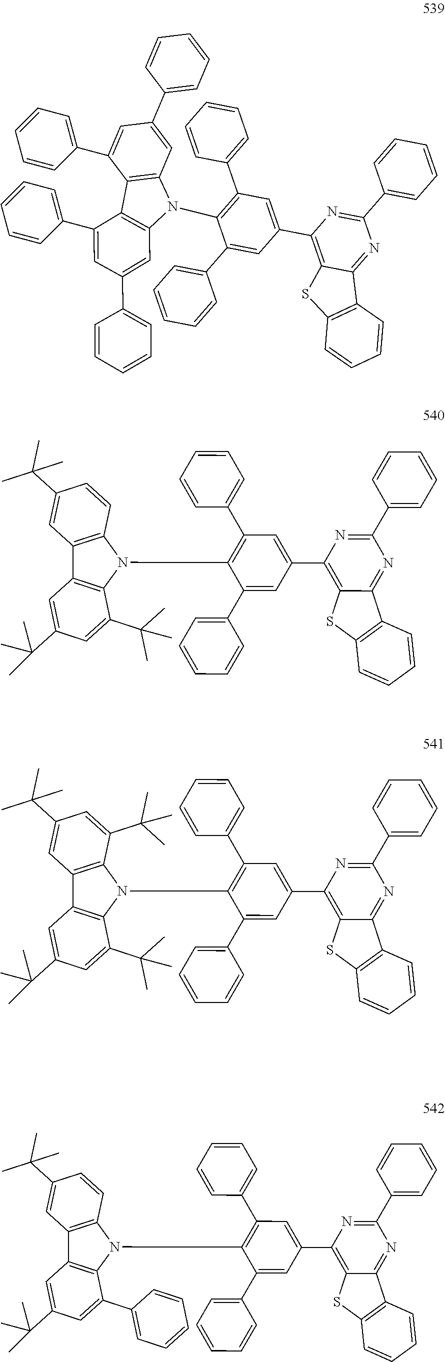 [SMILES: c1ccc(-c2cc(-c3ccccc3)c3c4c(-c5ccccc5)cc(-c5ccccc5)cc4n(-c4c(-c5ccccc5)cc(-c5nc(-c6ccccc6)nc6c5sc5ccccc56)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc3c2sc2ccccc23)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc3c2sc2ccccc23)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)nc3c2sc2ccccc23)cc1-c1ccccc1]

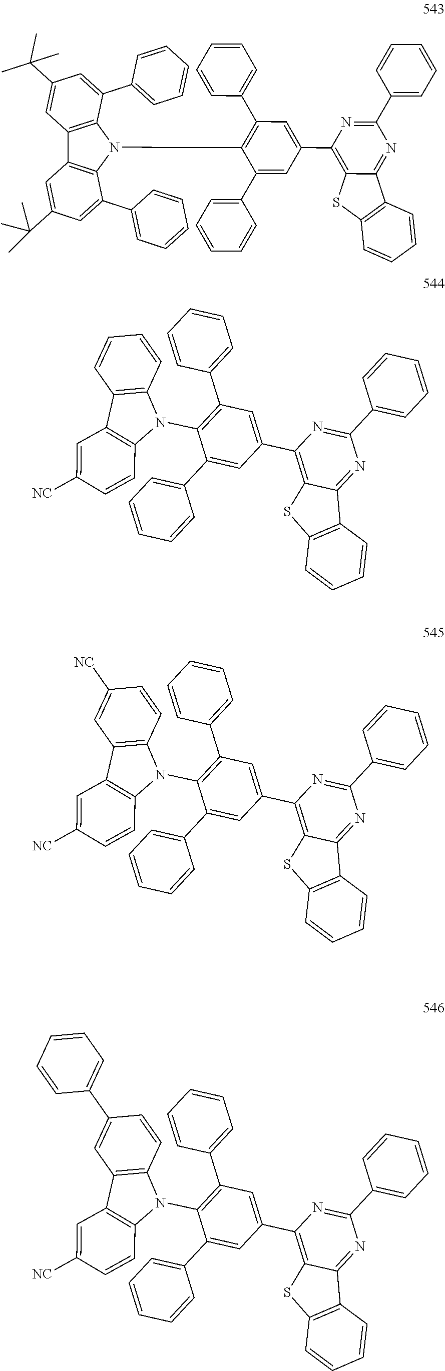 [SMILES: CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc3c2sc2ccccc23)cc1-c1ccccc1 ; N#Cc1ccc2c(c1)c1ccccc1n2-c1c(-c2ccccc2)cc(-c2nc(-c3ccccc3)nc3c2sc2ccccc23)cc1-c1ccccc1 ; N#Cc1ccc2c(c1)c1cc(C#N)ccc1n2-c1c(-c2ccccc2)cc(-c2nc(-c3ccccc3)nc3c2sc2ccccc23)cc1-c1ccccc1 ; N#Cc1ccc2c(c1)c1cc(-c3ccccc3)ccc1n2-c1c(-c2ccccc2)cc(-c2nc(-c3ccccc3)nc3c2sc2ccccc23)cc1-c1ccccc1]

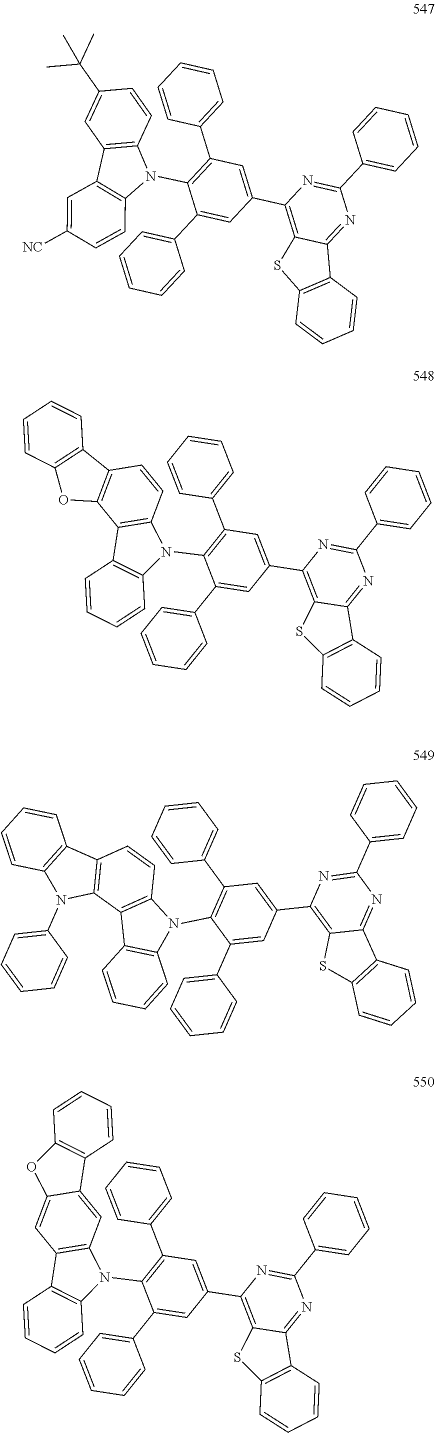 [SMILES: CC(C)(C)c1ccc2c(c1)c1cc(C#N)ccc1n2-c1c(-c2ccccc2)cc(-c2nc(-c3ccccc3)nc3c2sc2ccccc23)cc1-c1ccccc1 ; c1ccc(-c2nc(-c3cc(-c4ccccc4)c(-n4c5ccccc5c5c6oc7ccccc7c6ccc54)c(-c4ccccc4)c3)c3sc4ccccc4c3n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)c3sc4ccccc4c3n2)cc1 ; c1ccc(-c2nc(-c3cc(-c4ccccc4)c(-n4c5ccccc5c5cc6oc7ccccc7c6cc54)c(-c4ccccc4)c3)c3sc4ccccc4c3n2)cc1]

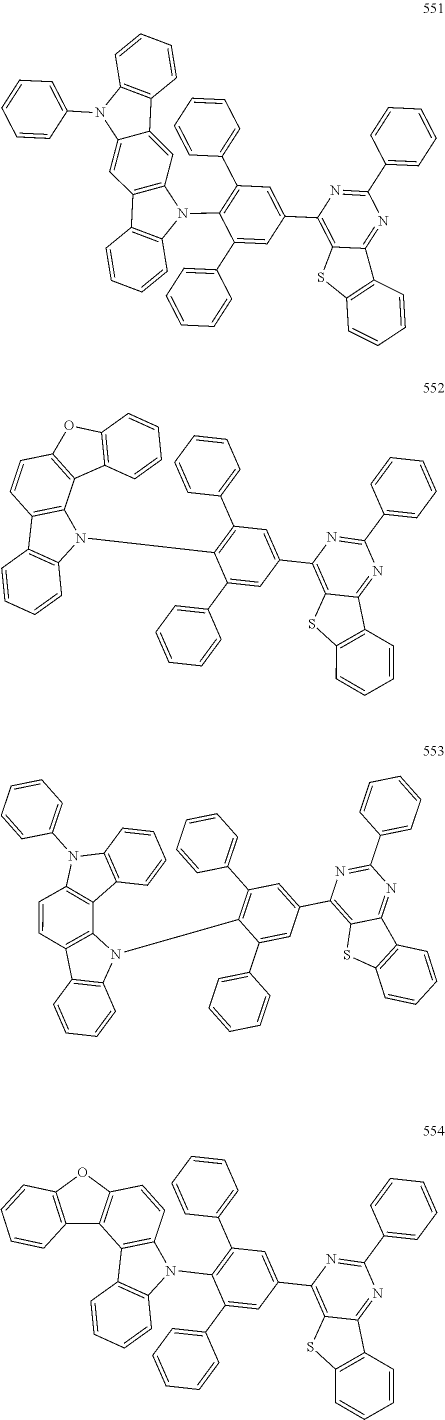 [SMILES: c1ccc(-c2nc(-c3cc(-c4ccccc4)c(-n4c5ccccc5c5cc6c(cc54)c4ccccc4n6-c4ccccc4)c(-c4ccccc4)c3)c3sc4ccccc4c3n2)cc1 ; c1ccc(-c2nc(-c3cc(-c4ccccc4)c(-n4c5ccccc5c5ccc6oc7ccccc7c6c54)c(-c4ccccc4)c3)c3sc4ccccc4c3n2)cc1 ; c1ccc(-c2nc(-c3cc(-c4ccccc4)c(-n4c5ccccc5c5ccc6c(c7ccccc7n6-c6ccccc6)c54)c(-c4ccccc4)c3)c3sc4ccccc4c3n2)cc1 ; c1ccc(-c2nc(-c3cc(-c4ccccc4)c(-n4c5ccccc5c5c6c(ccc54)oc4ccccc46)c(-c4ccccc4)c3)c3sc4ccccc4c3n2)cc1]

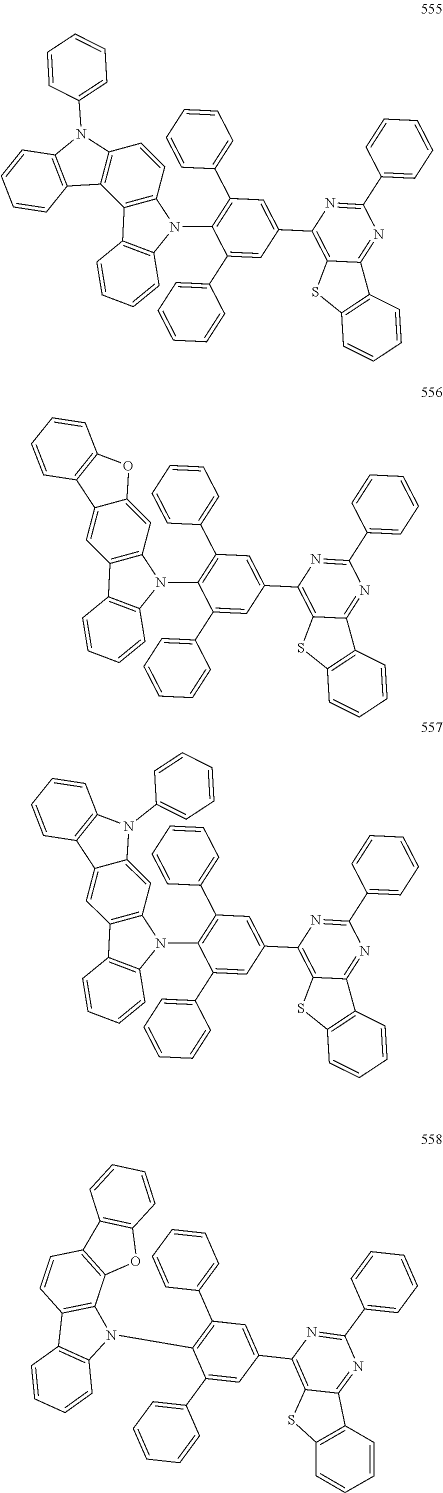 [SMILES: c1ccc(-c2nc(-c3cc(-c4ccccc4)c(-n4c5ccccc5c5c6c7ccccc7n(-c7ccccc7)c6ccc54)c(-c4ccccc4)c3)c3sc4ccccc4c3n2)cc1 ; c1ccc(-c2nc(-c3cc(-c4ccccc4)c(-n4c5ccccc5c5cc6c(cc54)oc4ccccc46)c(-c4ccccc4)c3)c3sc4ccccc4c3n2)cc1 ; c1ccc(-c2nc(-c3cc(-c4ccccc4)c(-n4c5ccccc5c5cc6c7ccccc7n(-c7ccccc7)c6cc54)c(-c4ccccc4)c3)c3sc4ccccc4c3n2)cc1 ; c1ccc(-c2nc(-c3cc(-c4ccccc4)c(-n4c5ccccc5c5ccc6c7ccccc7oc6c54)c(-c4ccccc4)c3)c3sc4ccccc4c3n2)cc1]

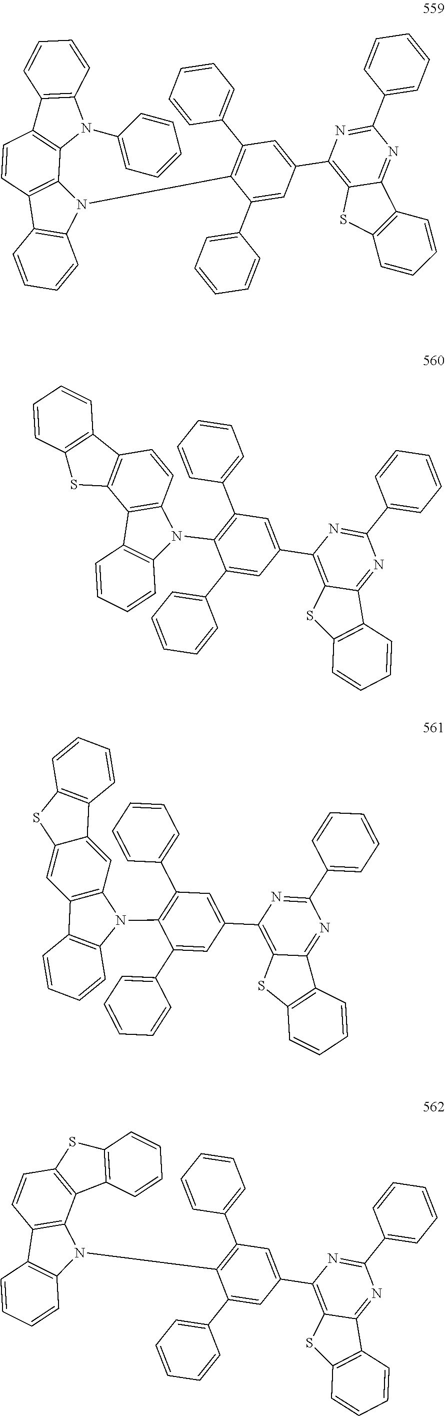 [SMILES: c1ccc(-c2nc(-c3cc(-c4ccccc4)c(-n4c5ccccc5c5ccc6c7ccccc7n(-c7ccccc7)c6c54)c(-c4ccccc4)c3)c3sc4ccccc4c3n2)cc1 ; c1ccc(-c2nc(-c3cc(-c4ccccc4)c(-n4c5ccccc5c5c6sc7ccccc7c6ccc54)c(-c4ccccc4)c3)c3sc4ccccc4c3n2)cc1 ; c1ccc(-c2nc(-c3cc(-c4ccccc4)c(-n4c5ccccc5c5cc6sc7ccccc7c6cc54)c(-c4ccccc4)c3)c3sc4ccccc4c3n2)cc1 ; c1ccc(-c2nc(-c3cc(-c4ccccc4)c(-n4c5ccccc5c5ccc6sc7ccccc7c6c54)c(-c4ccccc4)c3)c3sc4ccccc4c3n2)cc1]

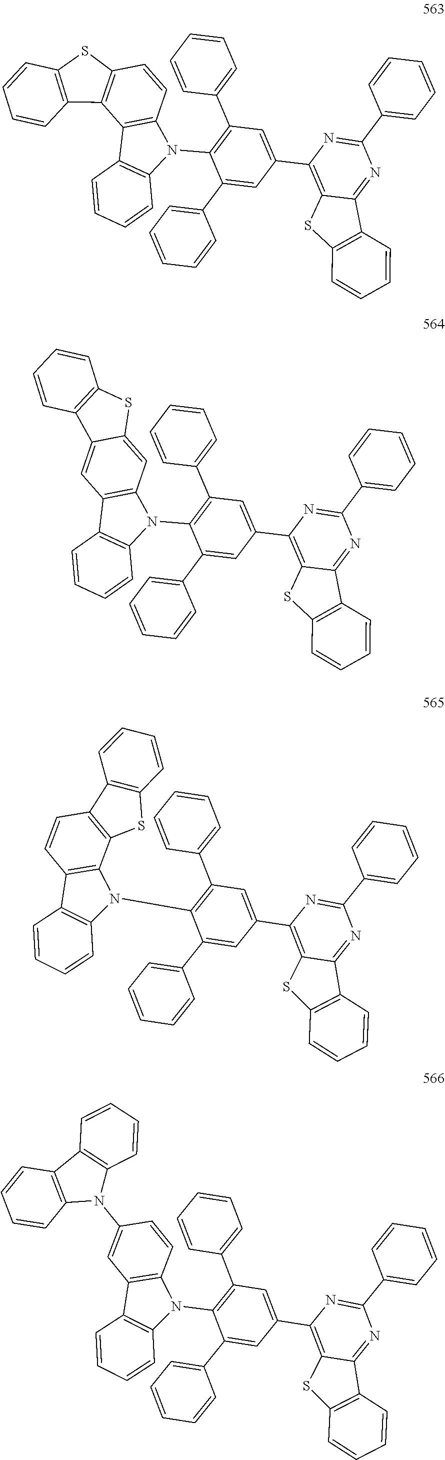 [SMILES: c1ccc(-c2nc(-c3cc(-c4ccccc4)c(-n4c5ccccc5c5c6c(ccc54)sc4ccccc46)c(-c4ccccc4)c3)c3sc4ccccc4c3n2)cc1 ; c1ccc(-c2nc(-c3cc(-c4ccccc4)c(-n4c5ccccc5c5cc6c(cc54)sc4ccccc46)c(-c4ccccc4)c3)c3sc4ccccc4c3n2)cc1 ; c1ccc(-c2nc(-c3cc(-c4ccccc4)c(-n4c5ccccc5c5ccc6c7ccccc7sc6c54)c(-c4ccccc4)c3)c3sc4ccccc4c3n2)cc1 ; c1ccc(-c2nc(-c3cc(-c4ccccc4)c(-n4c5ccccc5c5cc(-n6c7ccccc7c7ccccc76)ccc54)c(-c4ccccc4)c3)c3sc4ccccc4c3n2)cc1]

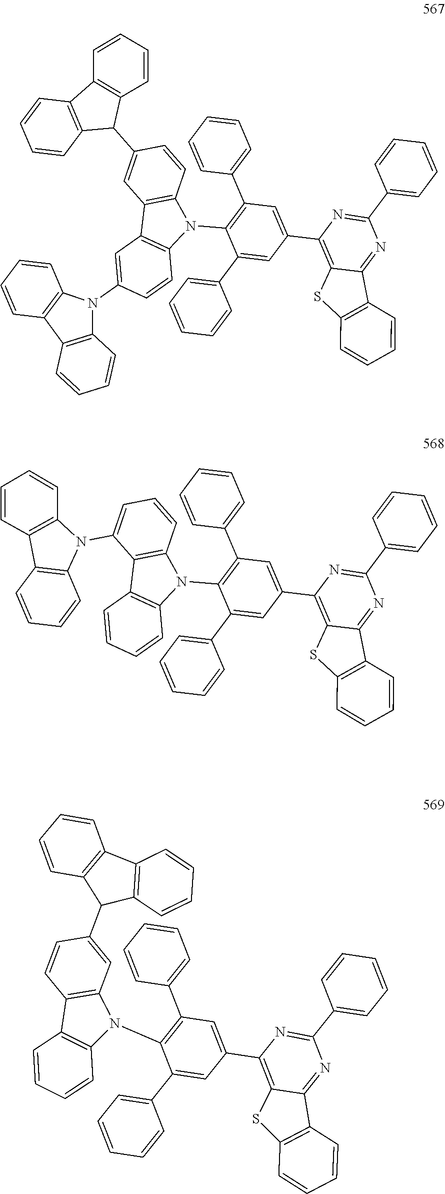 [SMILES: c1ccc(-c2nc(-c3cc(-c4ccccc4)c(-n4c5ccc(C6c7ccccc7-c7ccccc76)cc5c5cc(-n6c7ccccc7c7ccccc76)ccc54)c(-c4ccccc4)c3)c3sc4ccccc4c3n2)cc1 ; c1ccc(-c2nc(-c3cc(-c4ccccc4)c(-n4c5ccccc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5ccccc5c5ccc(C6c7ccccc7-c7ccccc76)cc54)c(-c4ccccc4)c3)c3sc4ccccc4c3n2)cc1]

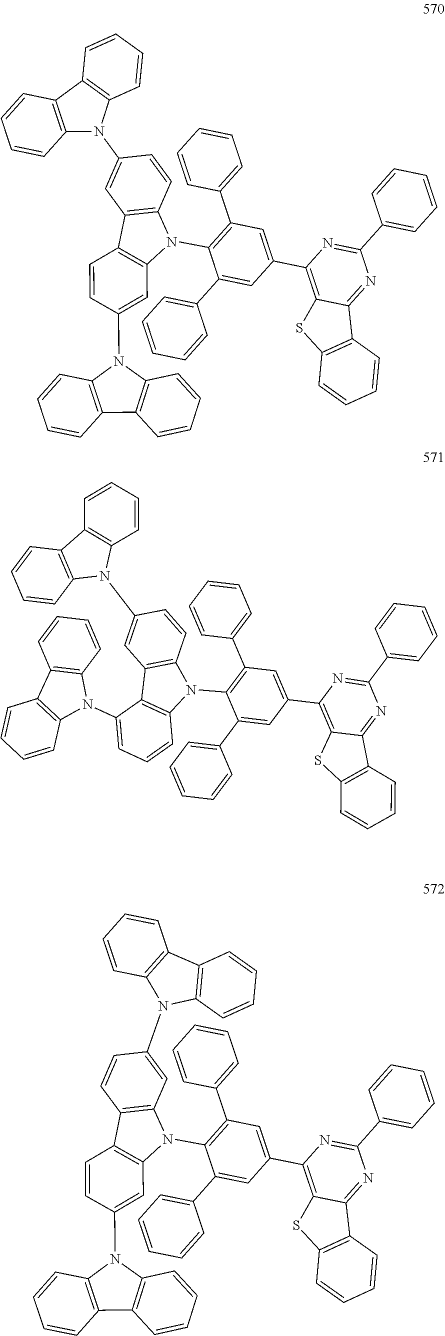 [SMILES: c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3sc4ccccc4c3n2)cc1]

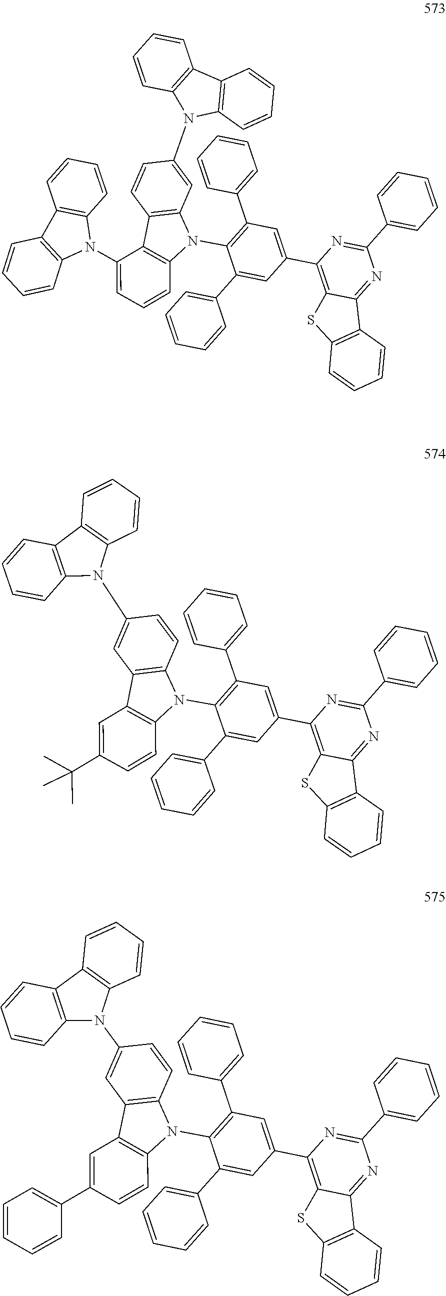 [SMILES: c1ccc(-c2nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)c3sc4ccccc4c3n2)cc1 ; CC(C)(C)c1ccc2c(c1)c1cc(-n3c4ccccc4c4ccccc43)ccc1n2-c1c(-c2ccccc2)cc(-c2nc(-c3ccccc3)nc3c2sc2ccccc23)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccccc4)nc4c3sc3ccccc34)cc2-c2ccccc2)cc1]

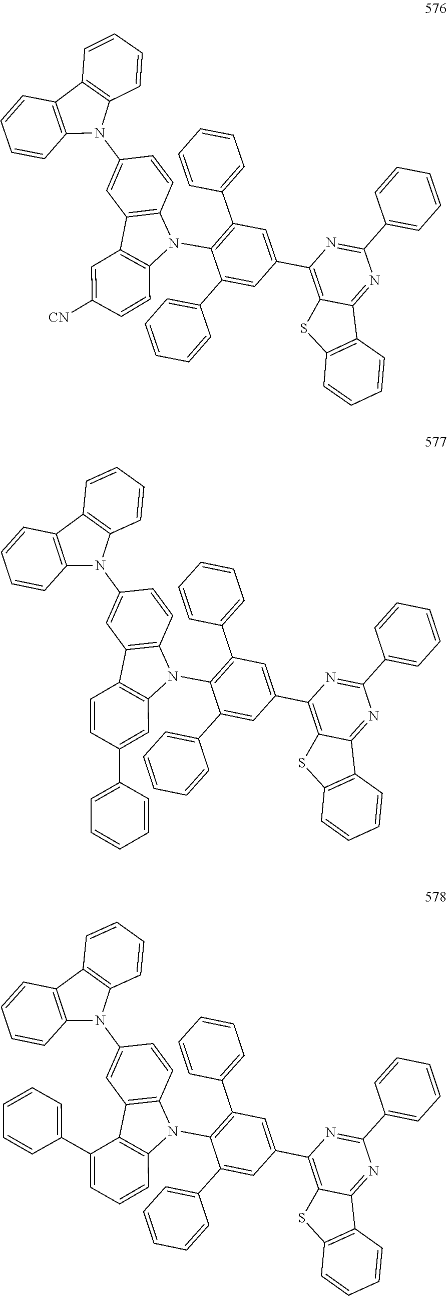

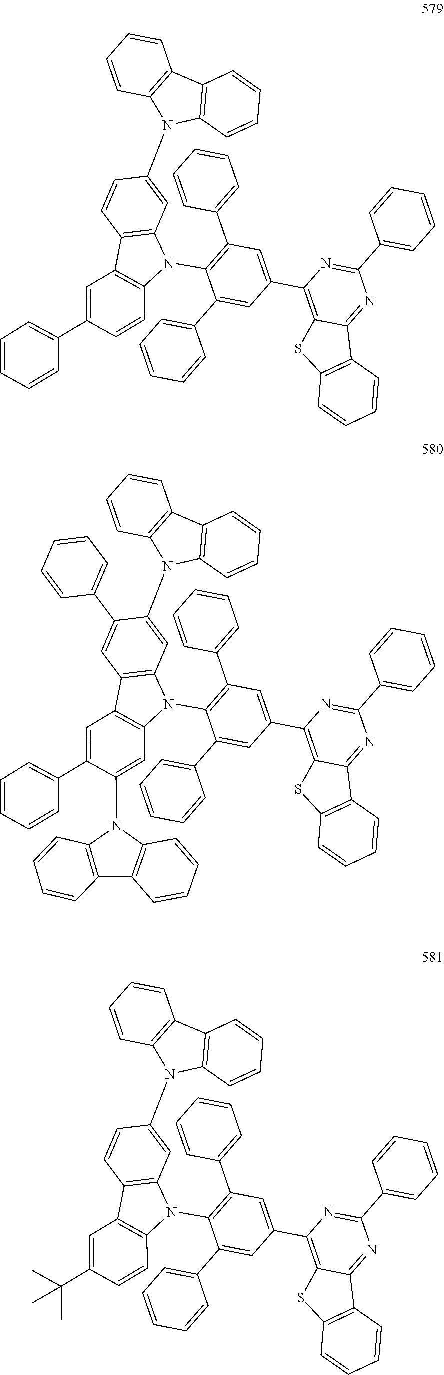 [SMILES: c1ccc(-c2ccc3c(c2)c2ccc(-n4c5ccccc5c5ccccc54)cc2n3-c2c(-c3ccccc3)cc(-c3nc(-c4ccccc4)nc4c3sc3ccccc34)cc2-c2ccccc2)cc1 ; c1ccc(-c2nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)c3sc4ccccc4c3n2)cc1 ; CC(C)(C)c1ccc2c(c1)c1ccc(-n3c4ccccc4c4ccccc43)cc1n2-c1c(-c2ccccc2)cc(-c2nc(-c3ccccc3)nc3c2sc2ccccc23)cc1-c1ccccc1]

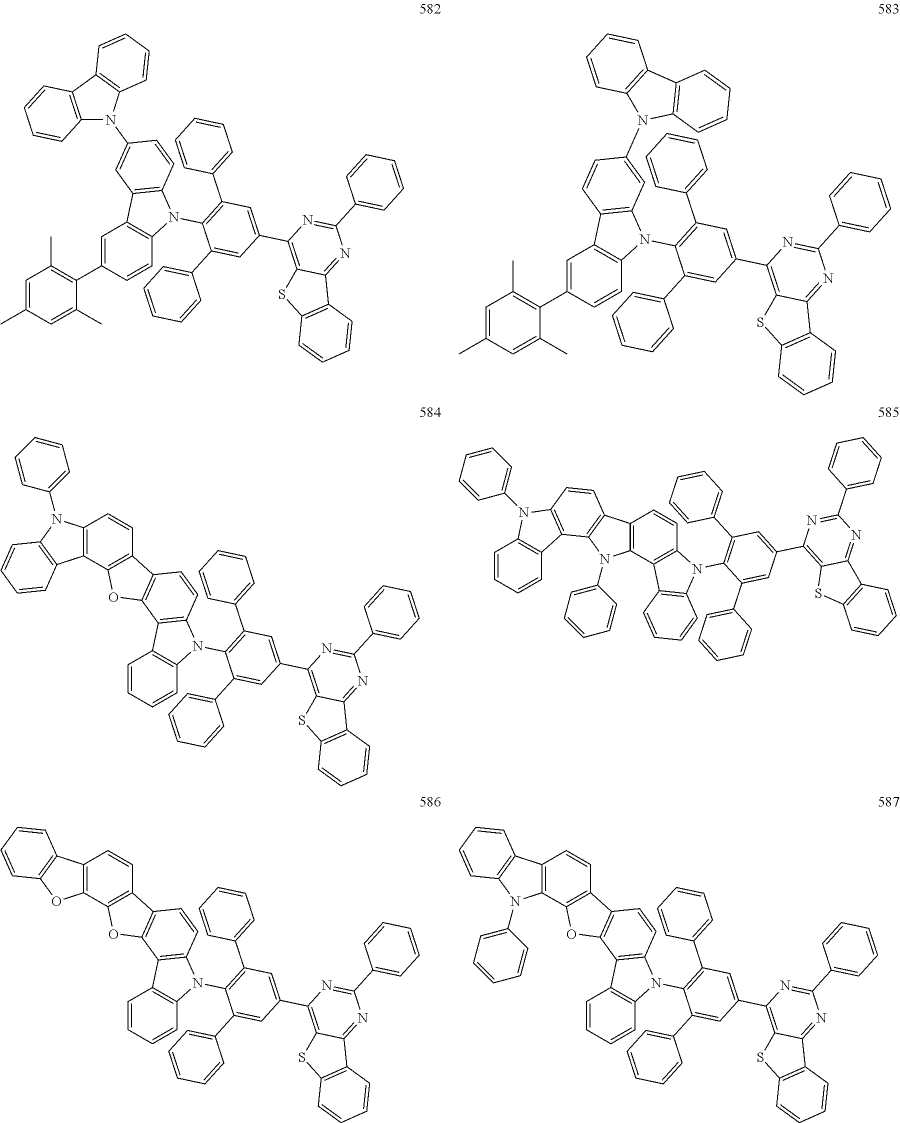

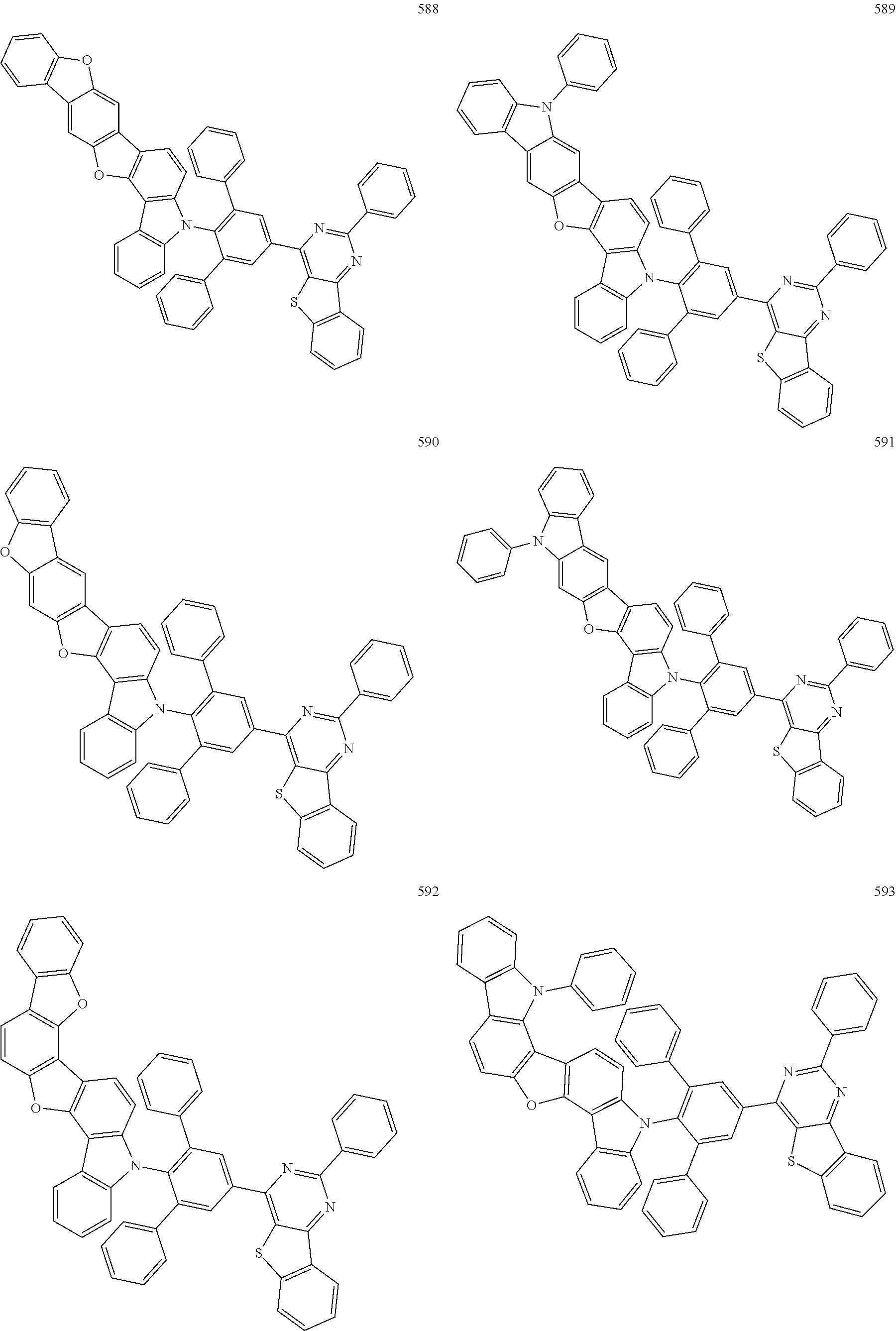

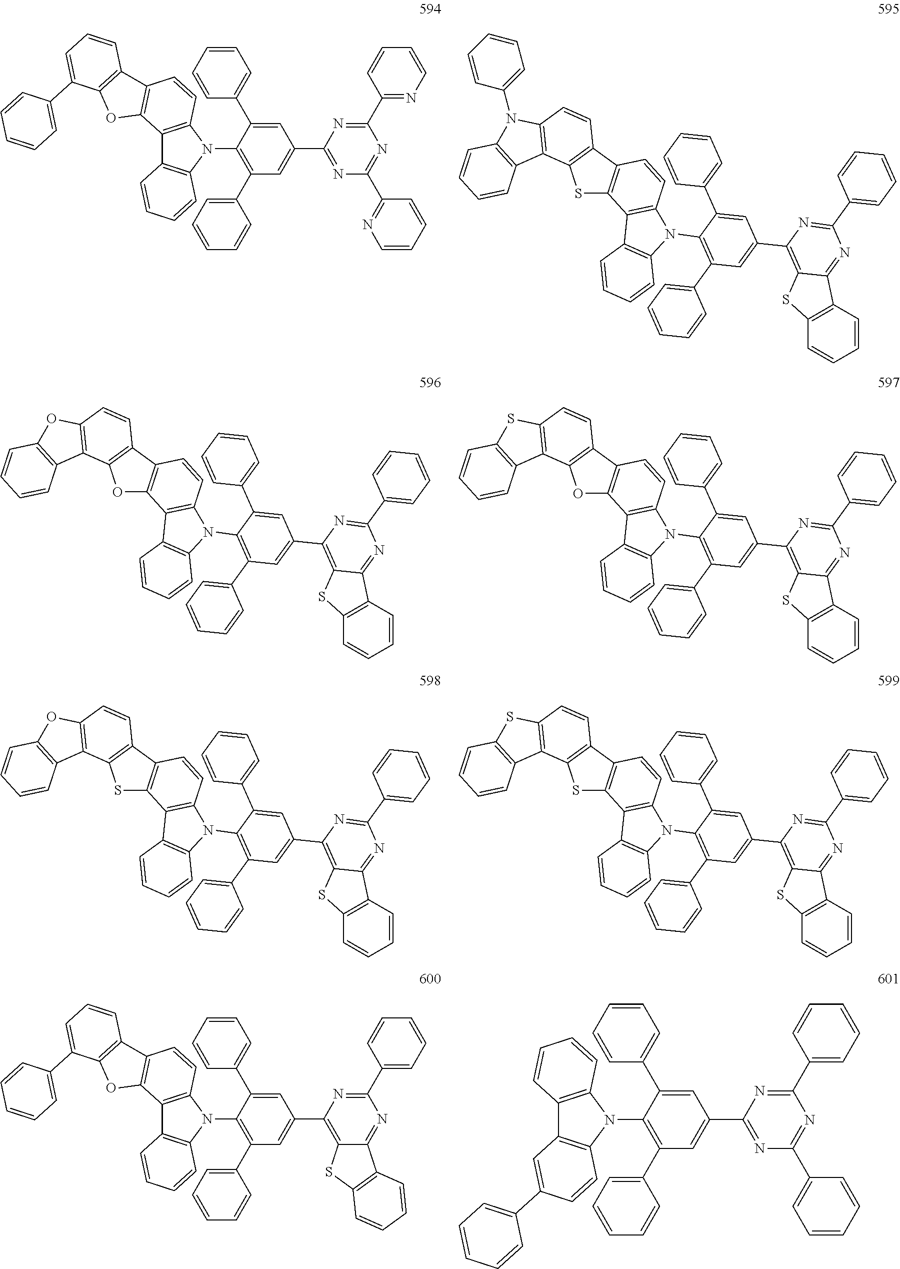

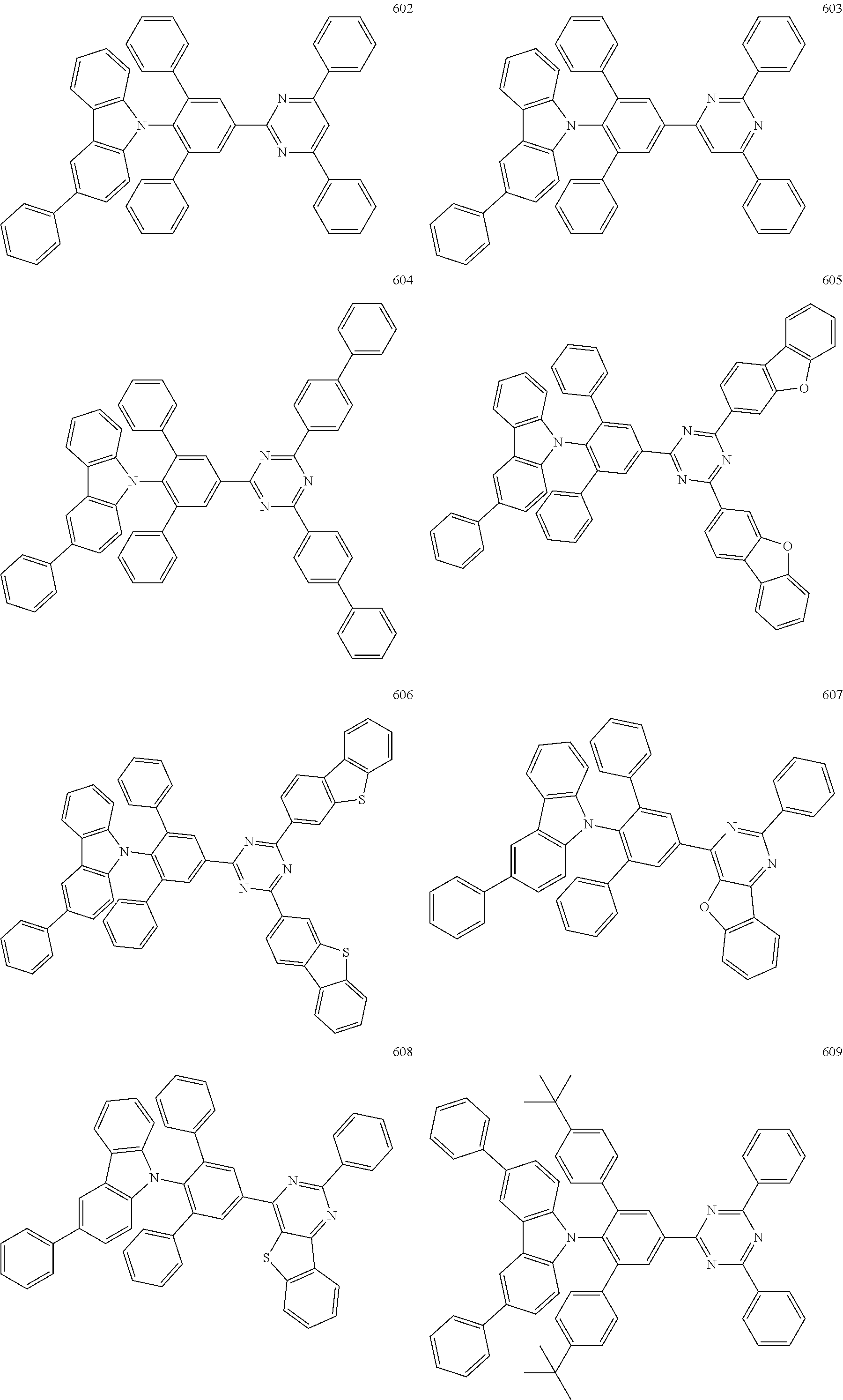

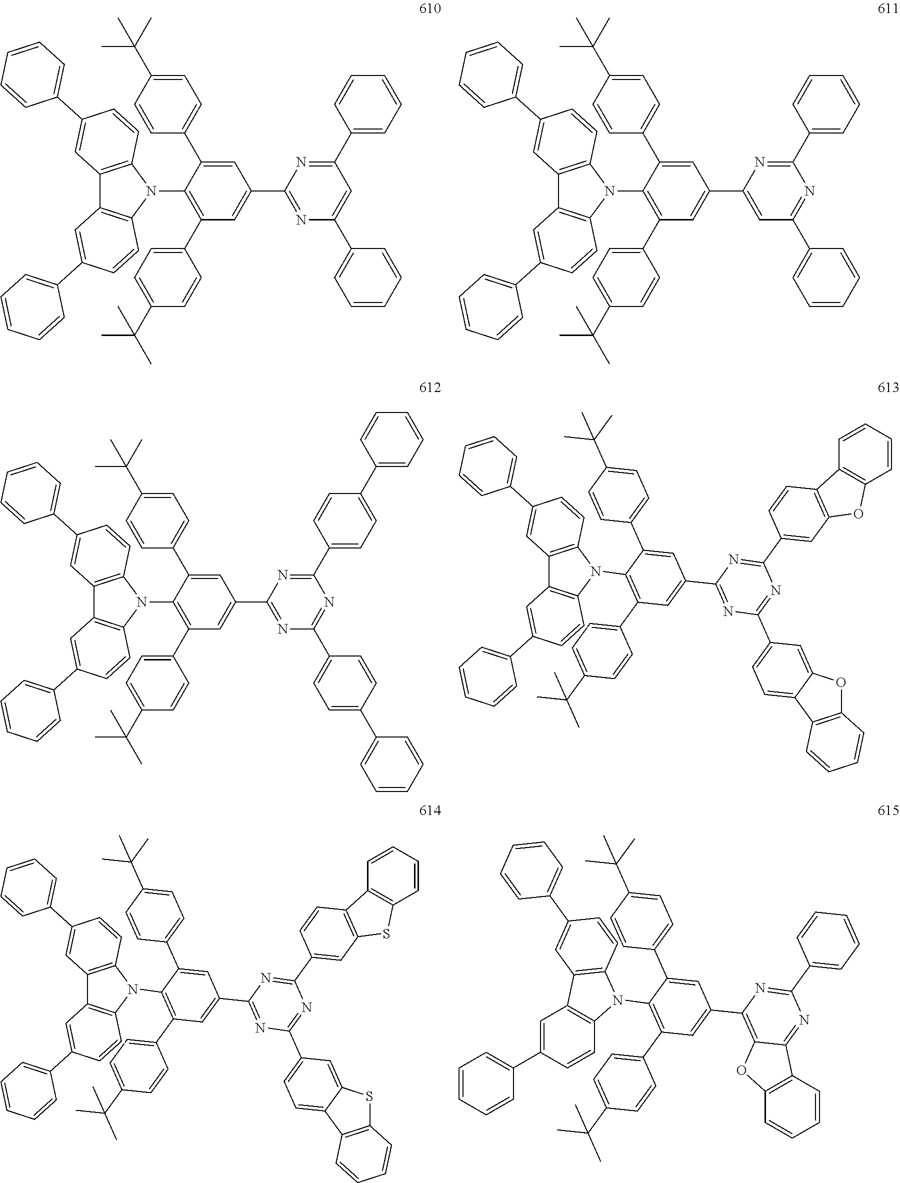

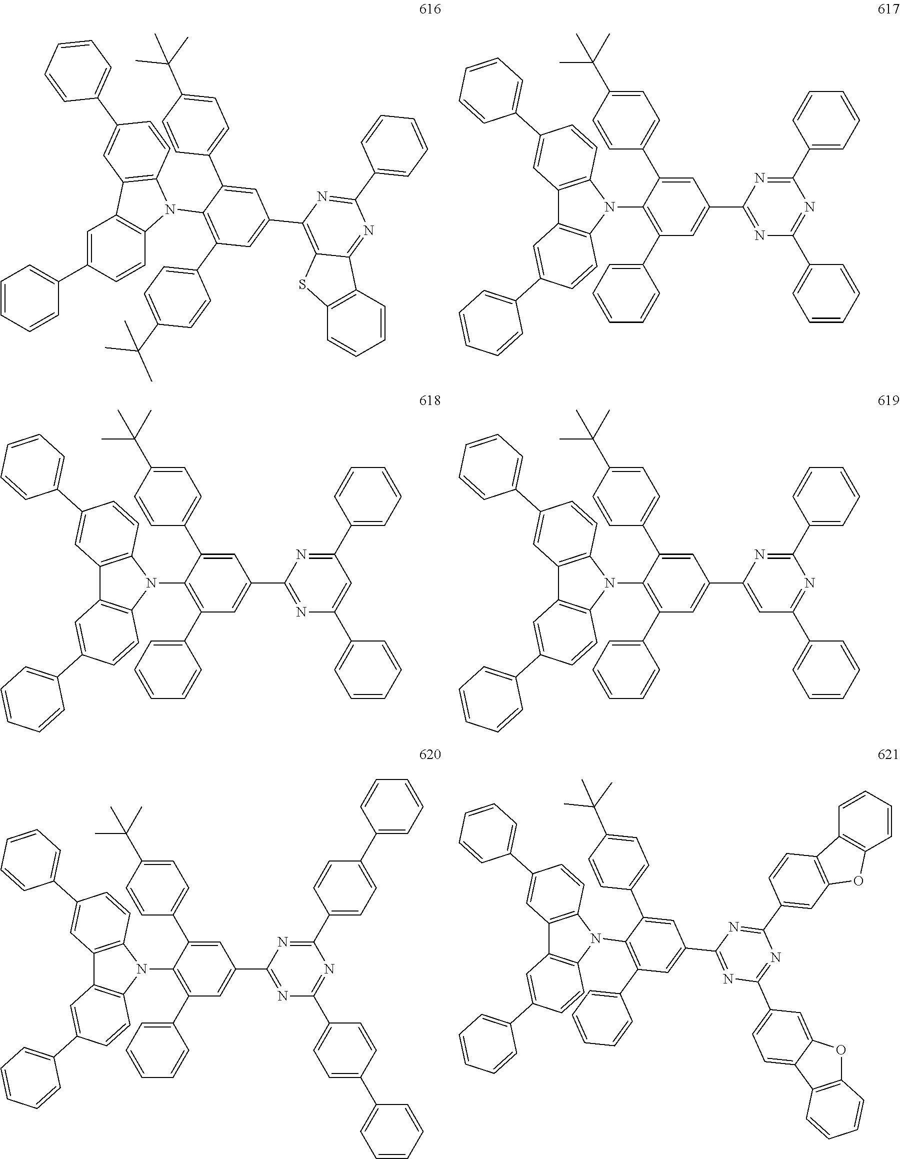

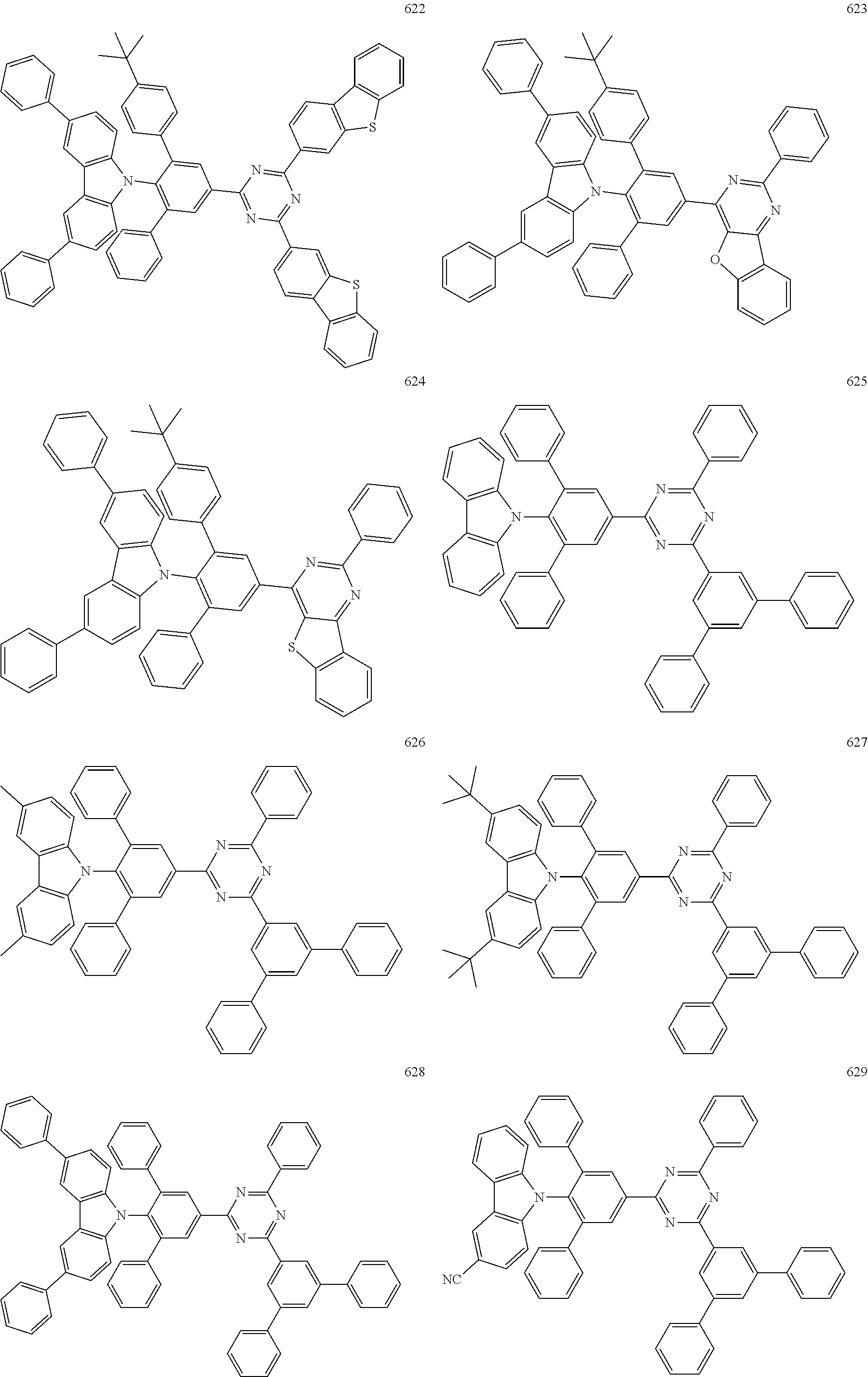

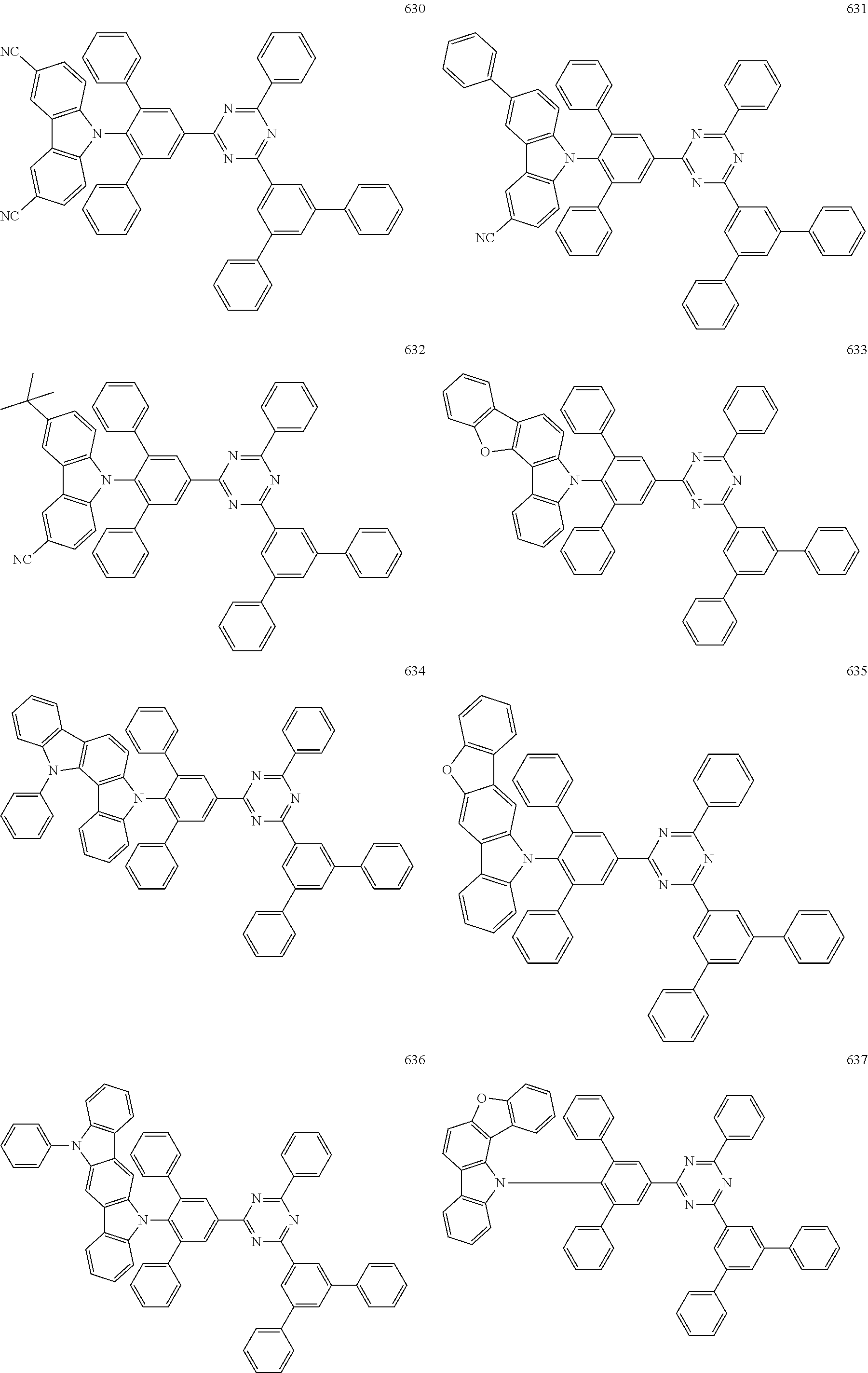

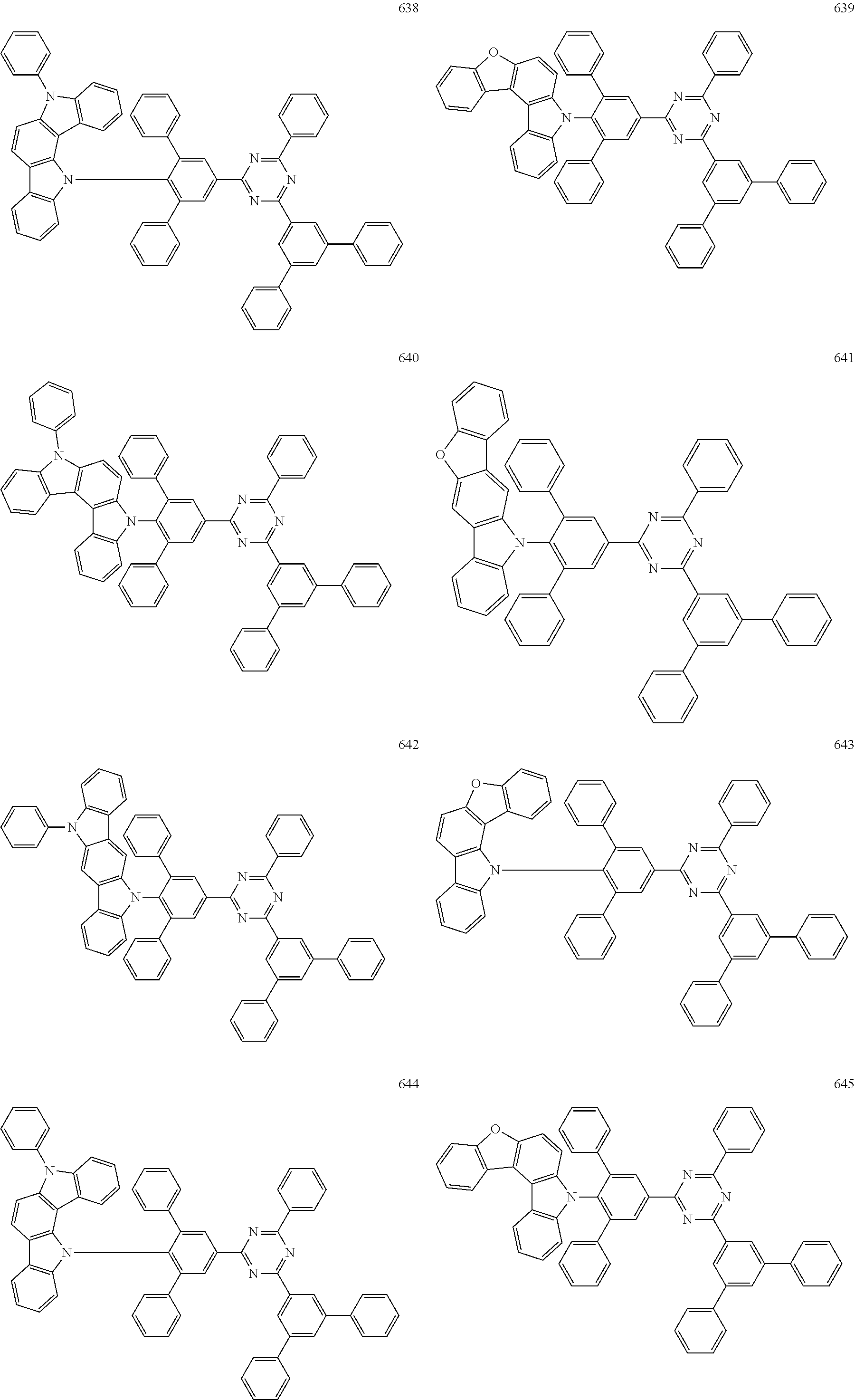

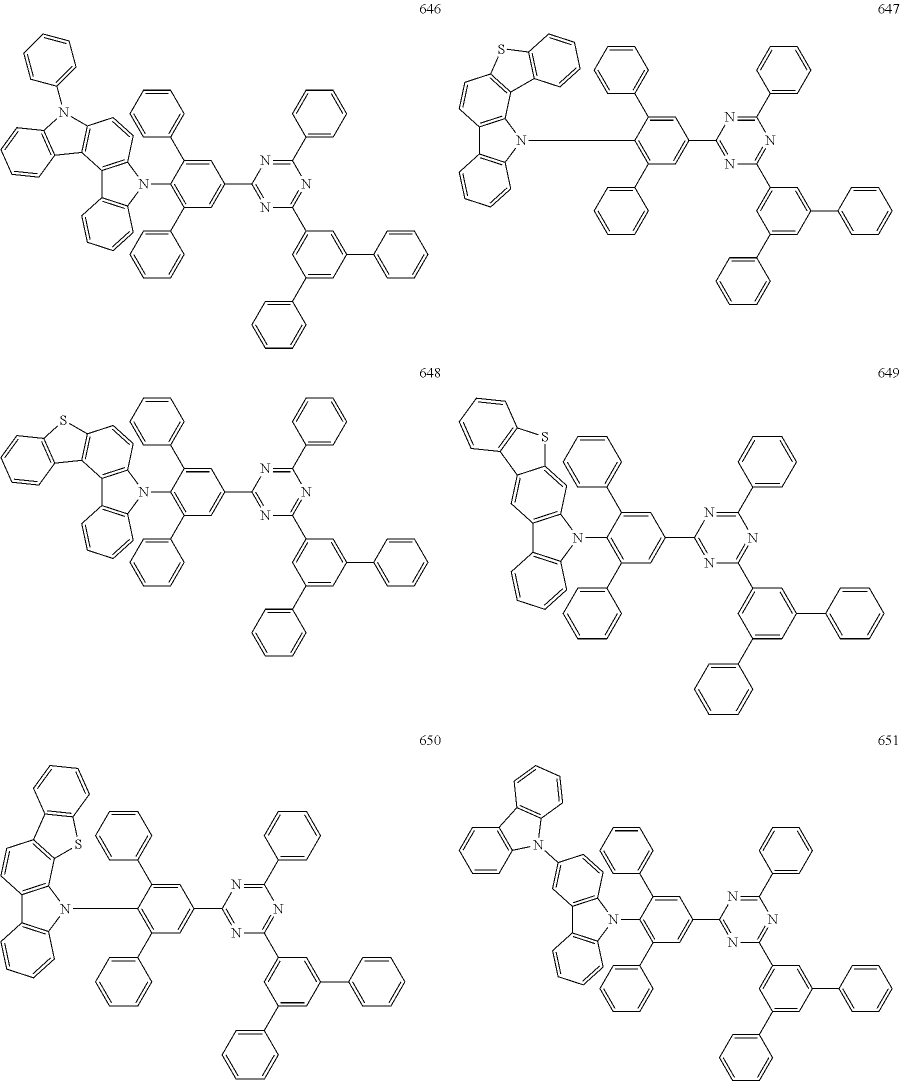

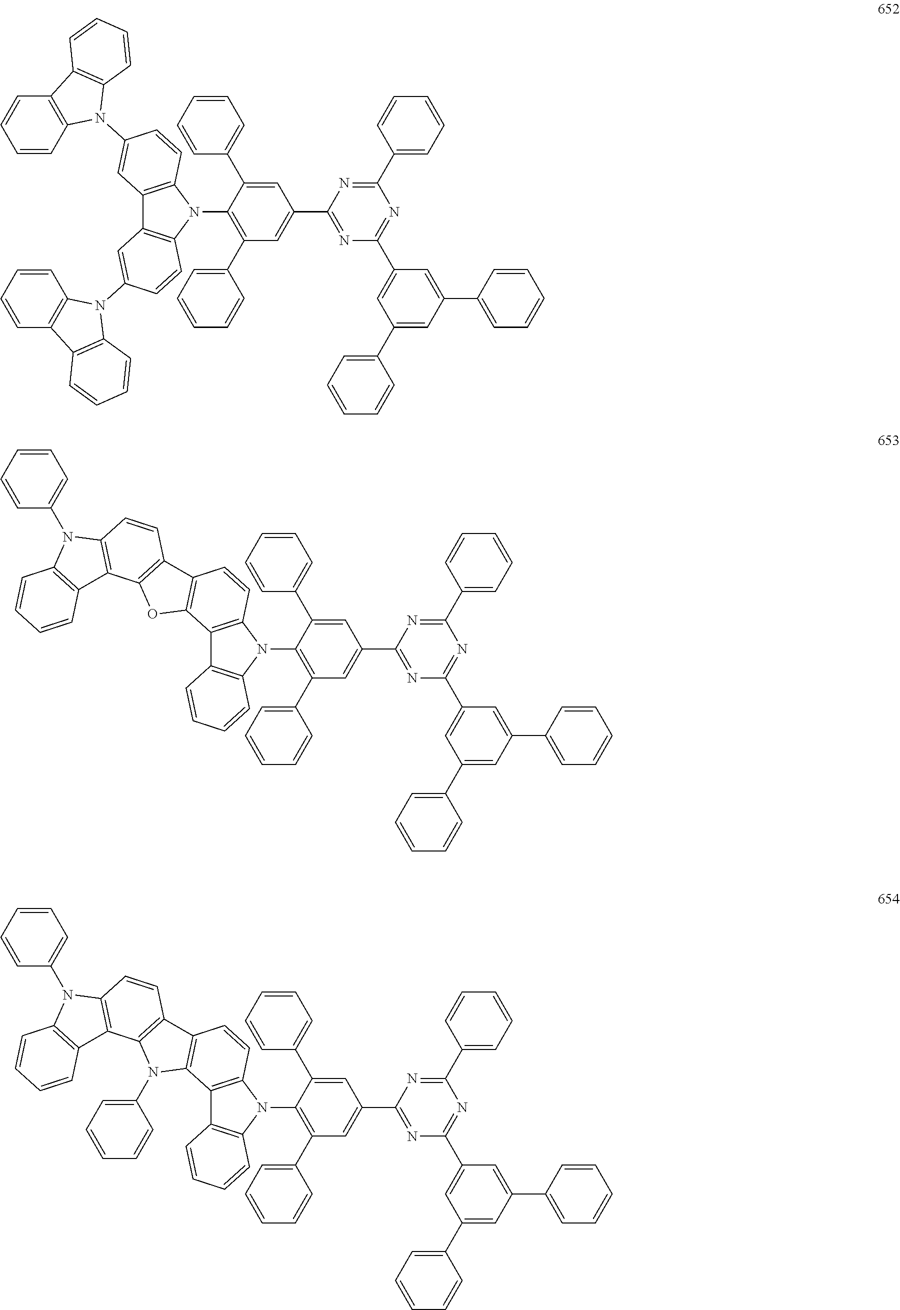 [SMILES: c1ccc(-c2cc(-c3ccccc3)cc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)c2)cc1]

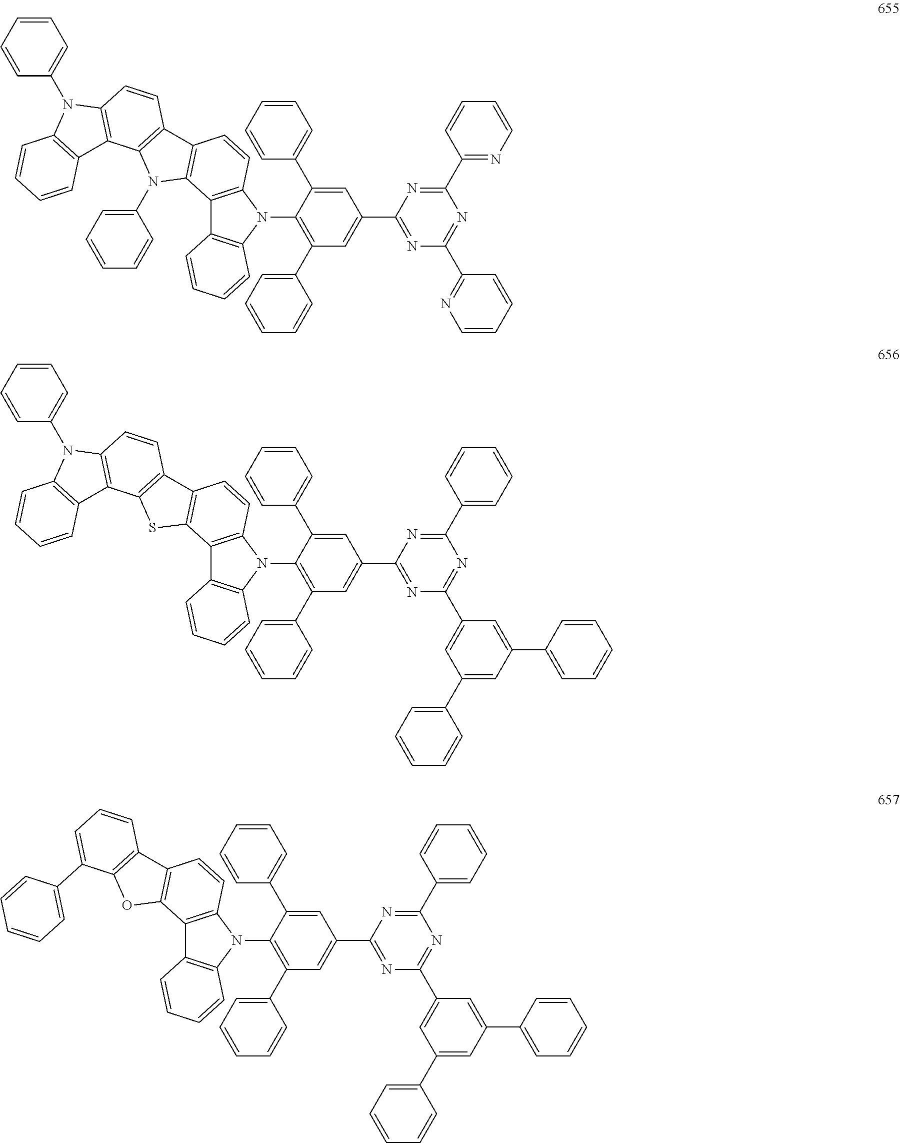 [SMILES: c1ccc(-c2cc(-c3nc(-c4ccccn4)nc(-c4ccccn4)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3ccccc3)cc(-c3nc(-c4ccccc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7oc8c(-c9ccccc9)cccc8c7ccc65)c(-c5ccccc5)c4)n3)c2)cc1]

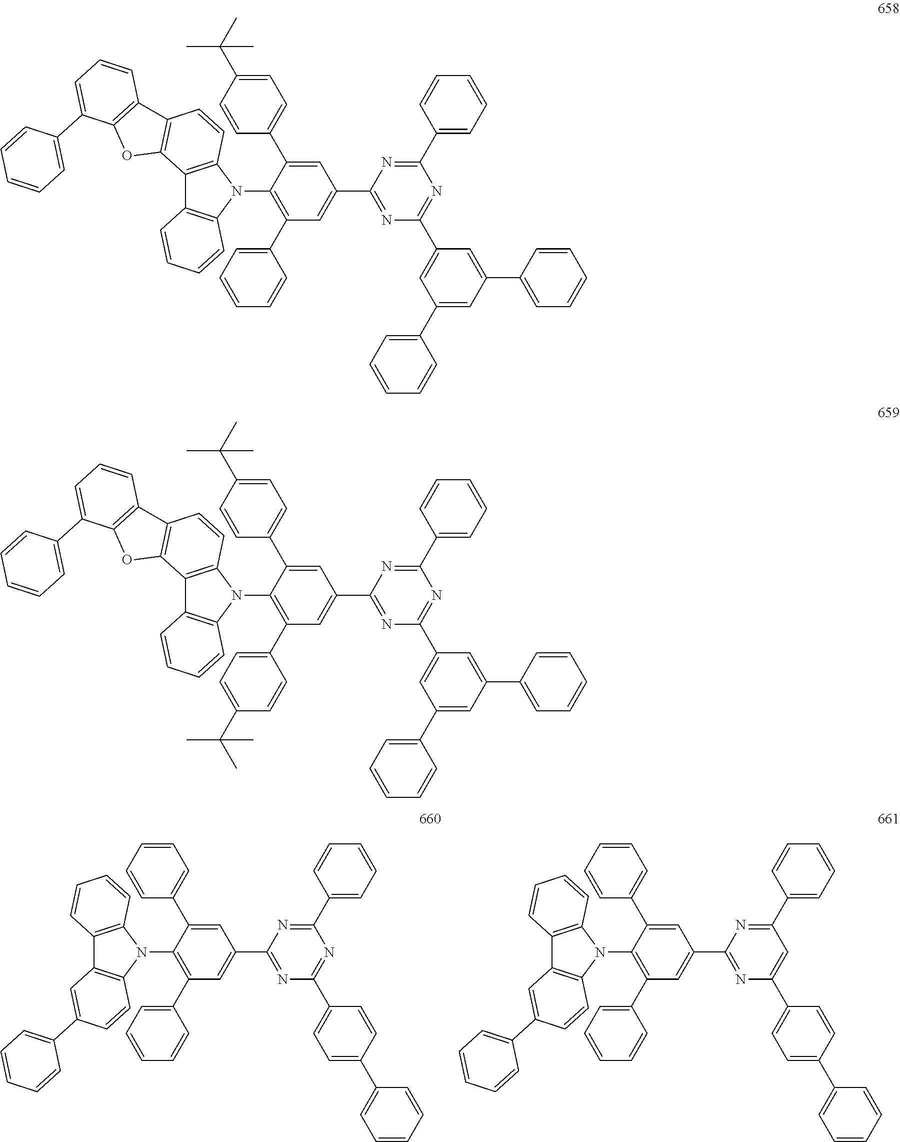

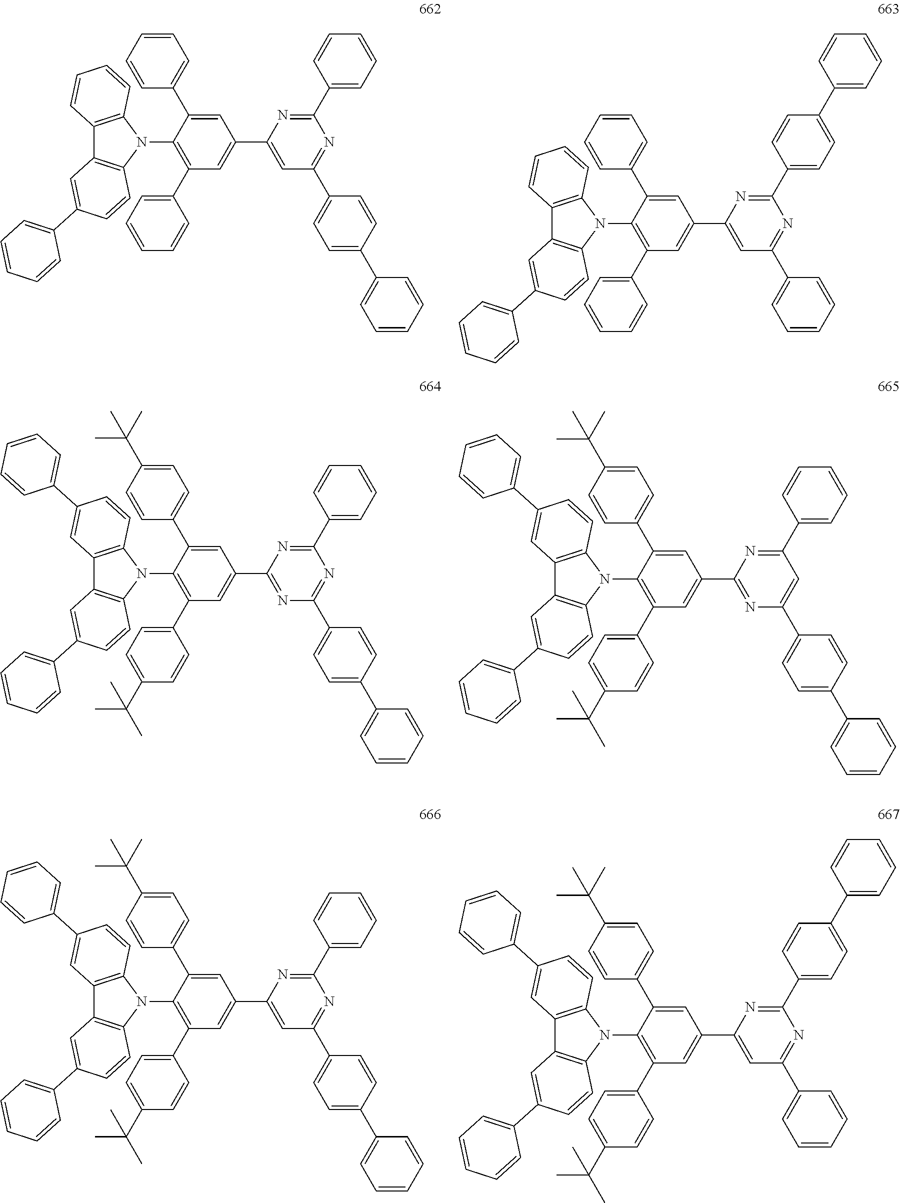

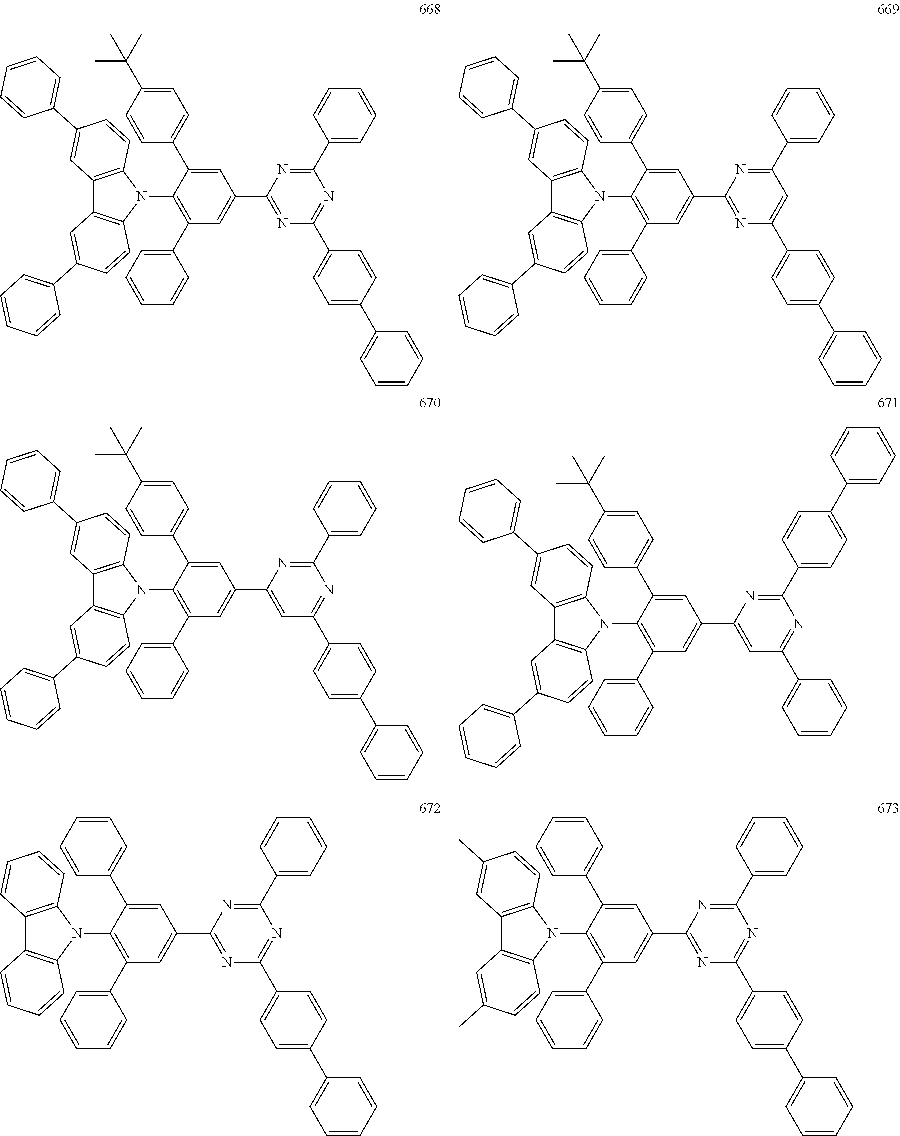

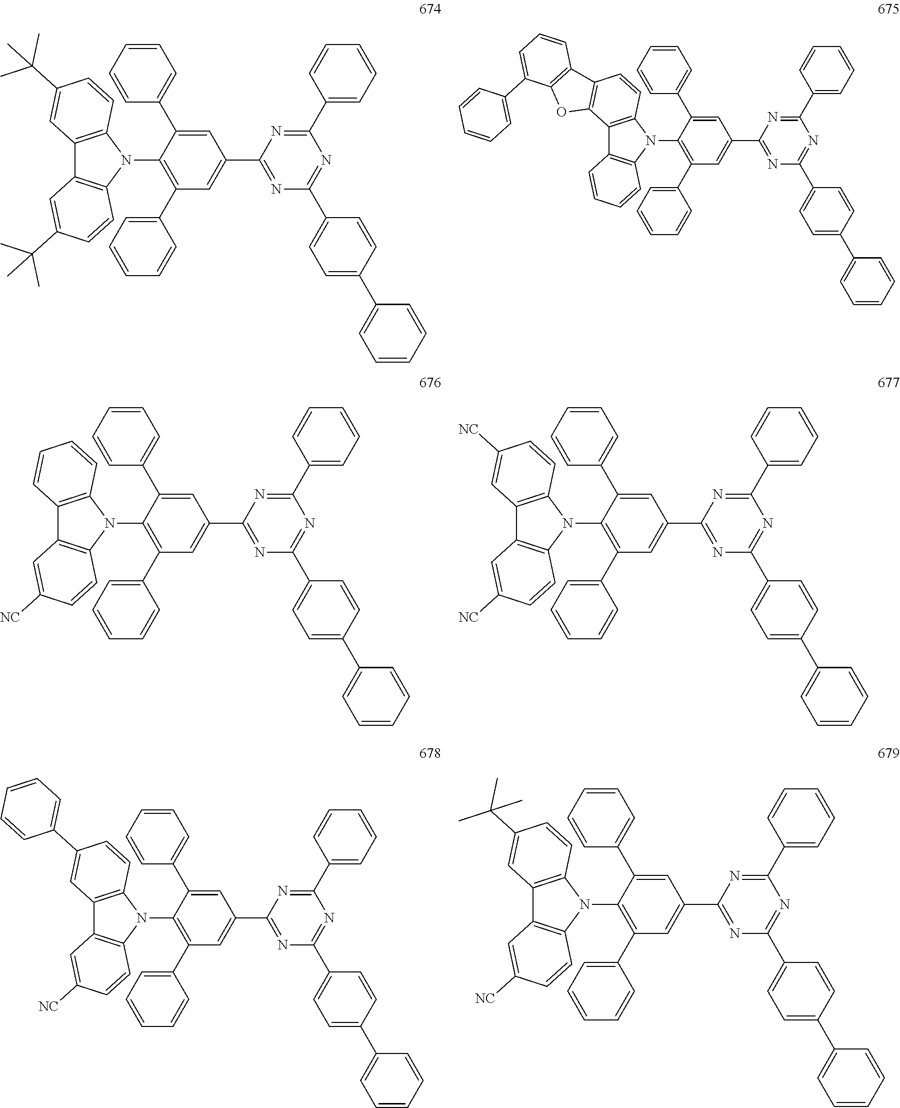

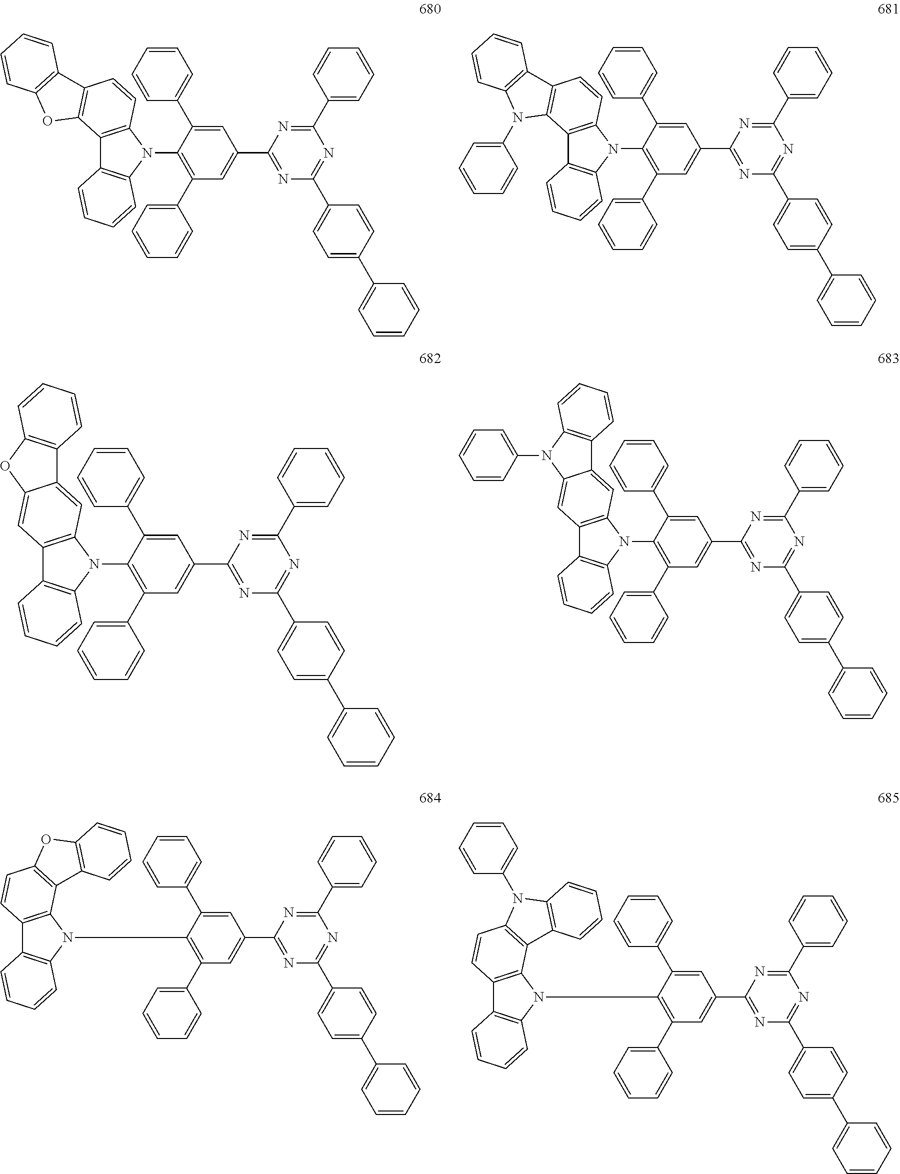

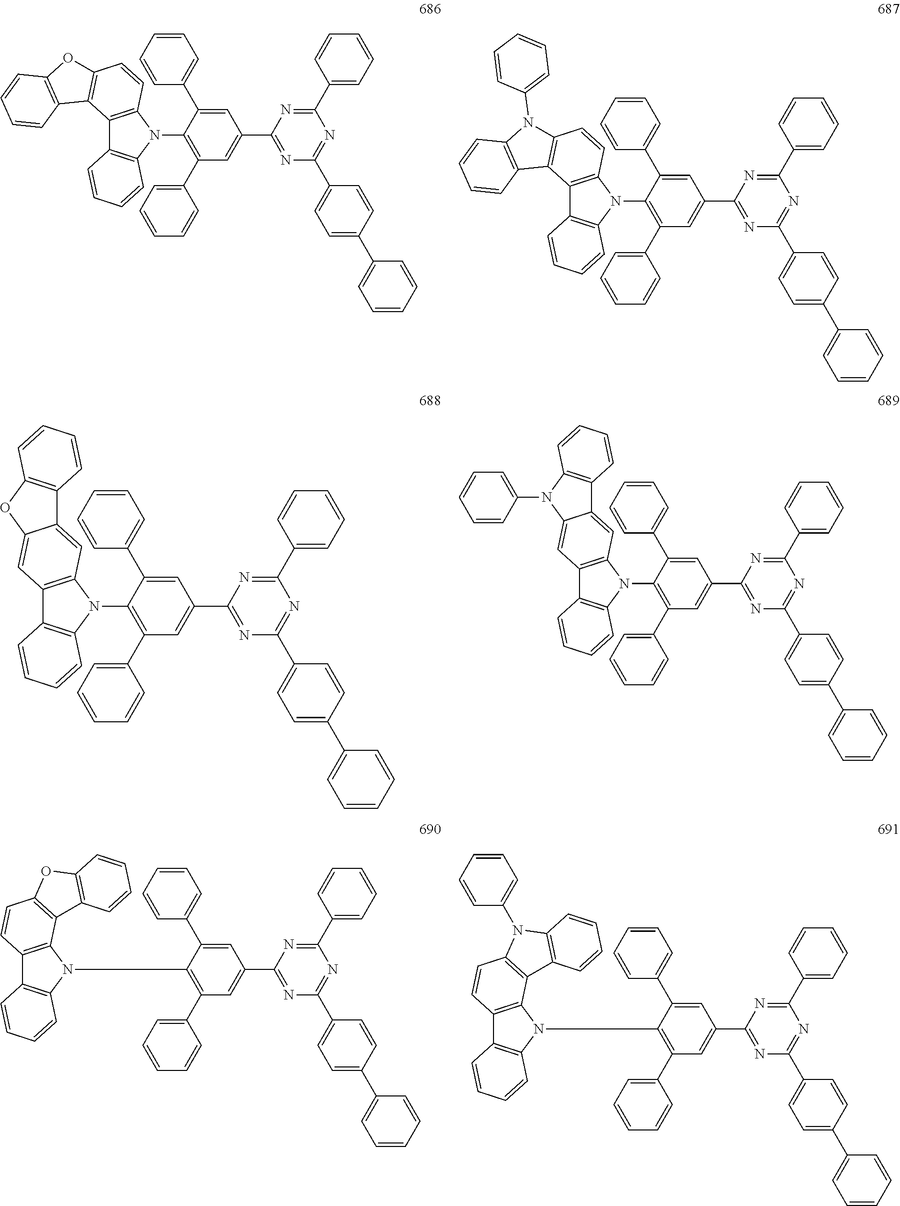

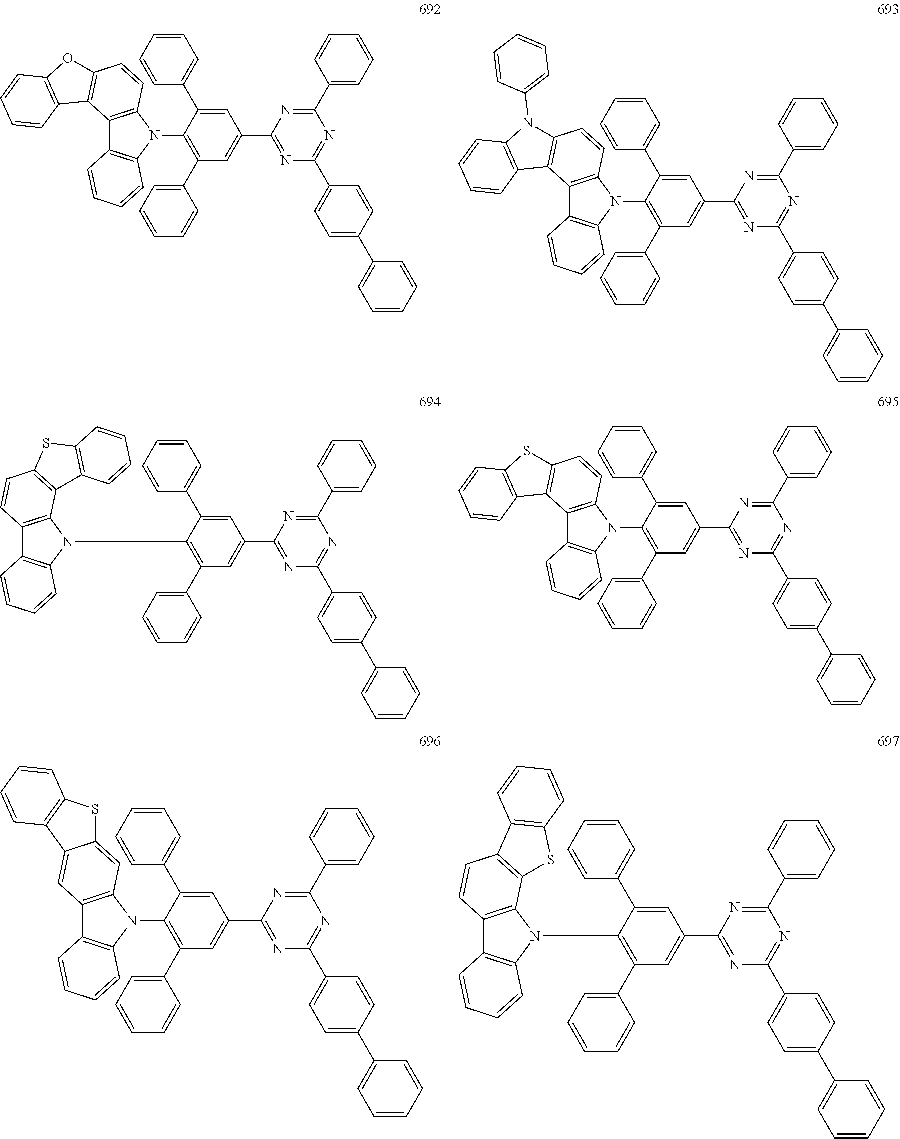

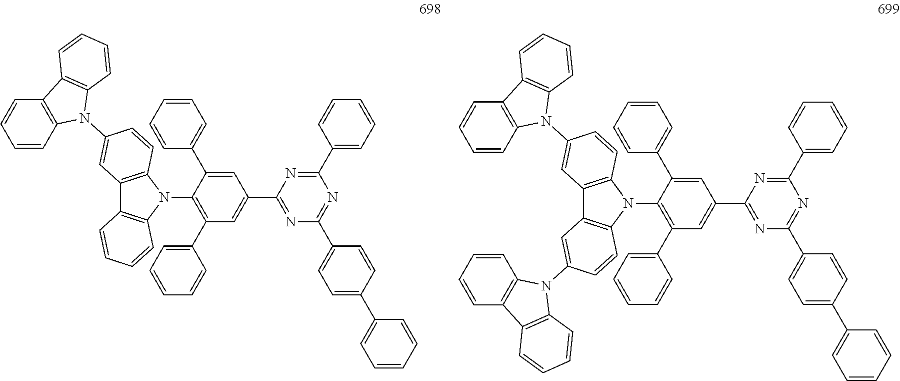 [SMILES: c1ccc(-c2ccc(-c3nc(-c4ccccc4)nc(-c4cc(-c5ccccc5)c(-n5c6ccccc6c6cc(-n7c8ccccc8c8ccccc87)ccc65)c(-c5ccccc5)c4)n3)cc2)cc1 ; c1ccc(-c2ccc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1]

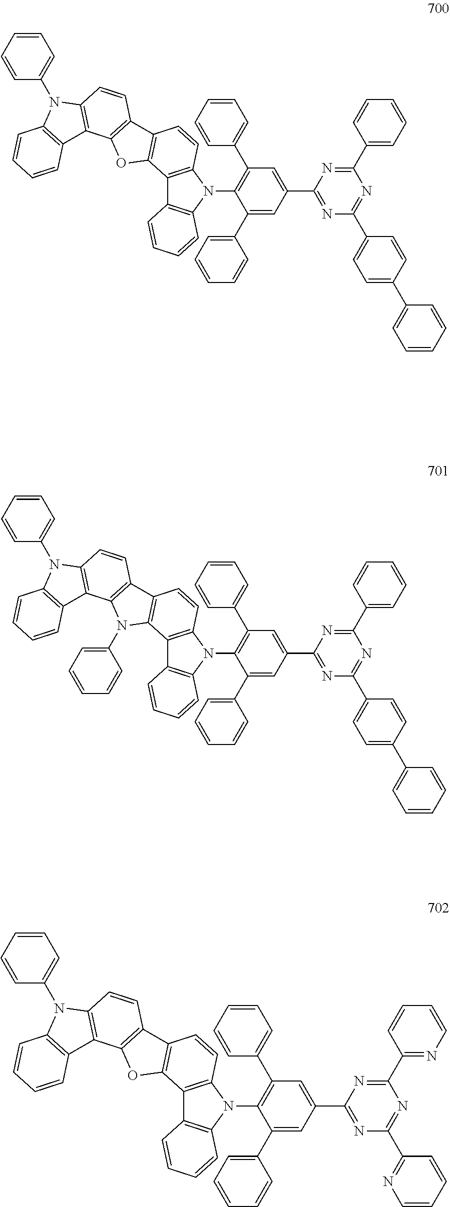 [SMILES: c1ccc(-c2ccc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1 ; c1ccc(-c2cc(-c3nc(-c4ccccn4)nc(-c4ccccn4)n3)cc(-c3ccccc3)c2-n2c3ccccc3c3c4oc5c(ccc6c5c5ccccc5n6-c5ccccc5)c4ccc32)cc1]

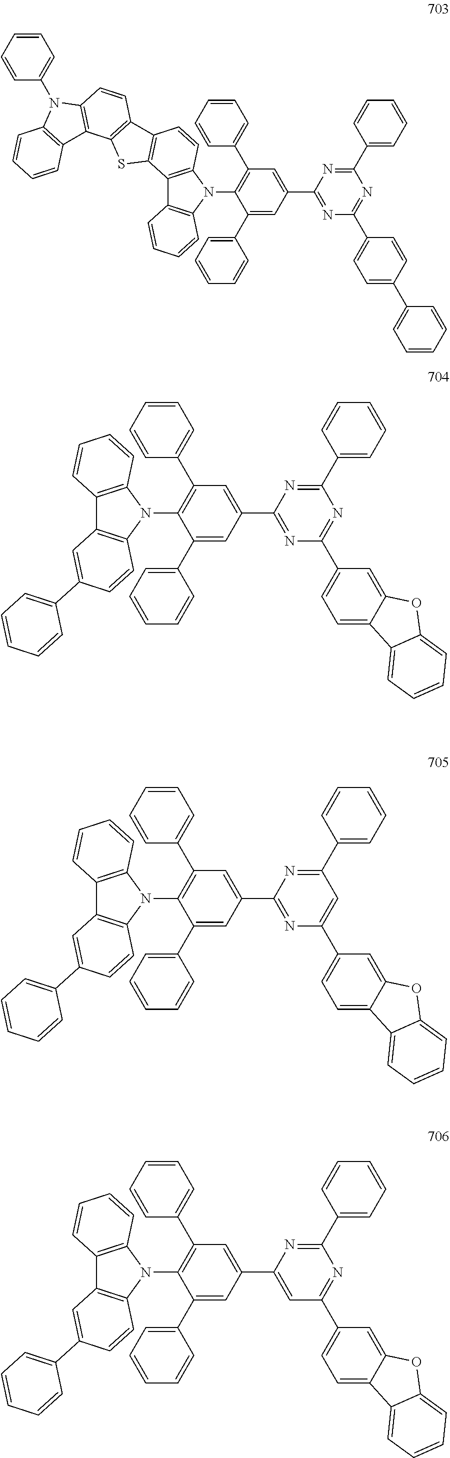 [SMILES: c1ccc(-c2ccc(-c3nc(-c4ccccc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1 ; c1ccc(-c2ccc3c(c2)c2ccccc2n3-c2c(-c3ccccc3)cc(-c3nc(-c4ccccc4)nc(-c4ccc5c(c4)oc4ccccc45)n3)cc2-c2ccccc2)cc1 ; c1ccc(-c2ccc3c(c2)c2ccccc2n3-c2c(-c3ccccc3)cc(-c3nc(-c4ccccc4)cc(-c4ccc5c(c4)oc4ccccc45)n3)cc2-c2ccccc2)cc1 ; c1ccc(-c2ccc3c(c2)c2ccccc2n3-c2c(-c3ccccc3)cc(-c3cc(-c4ccc5c(c4)oc4ccccc45)nc(-c4ccccc4)n3)cc2-c2ccccc2)cc1]

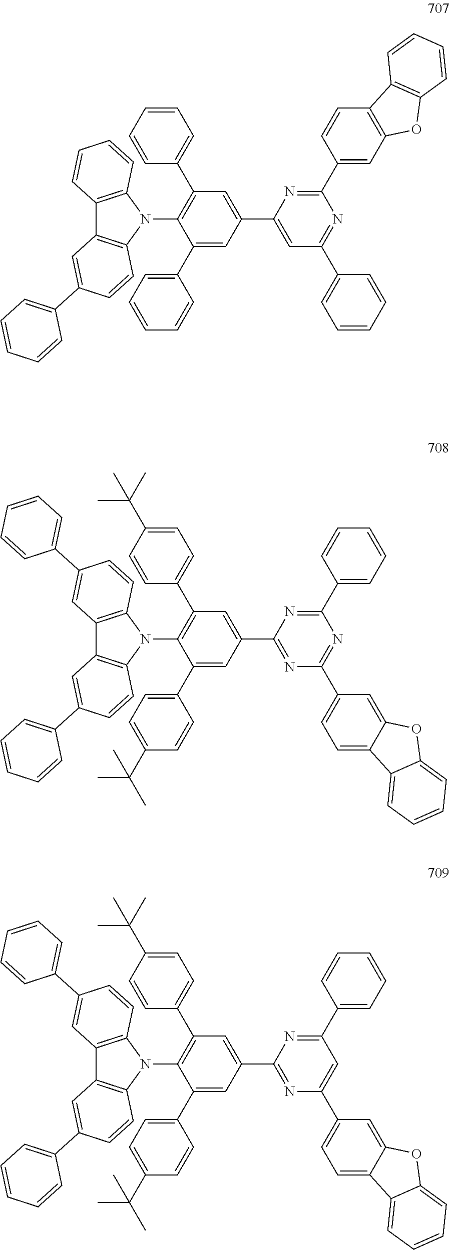 [SMILES: c1ccc(-c2ccc3c(c2)c2ccccc2n3-c2c(-c3ccccc3)cc(-c3cc(-c4ccccc4)nc(-c4ccc5c(c4)oc4ccccc45)n3)cc2-c2ccccc2)cc1 ; CC(C)(C)c1ccc(-c2cc(-c3nc(-c4ccccc4)nc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)cc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1]

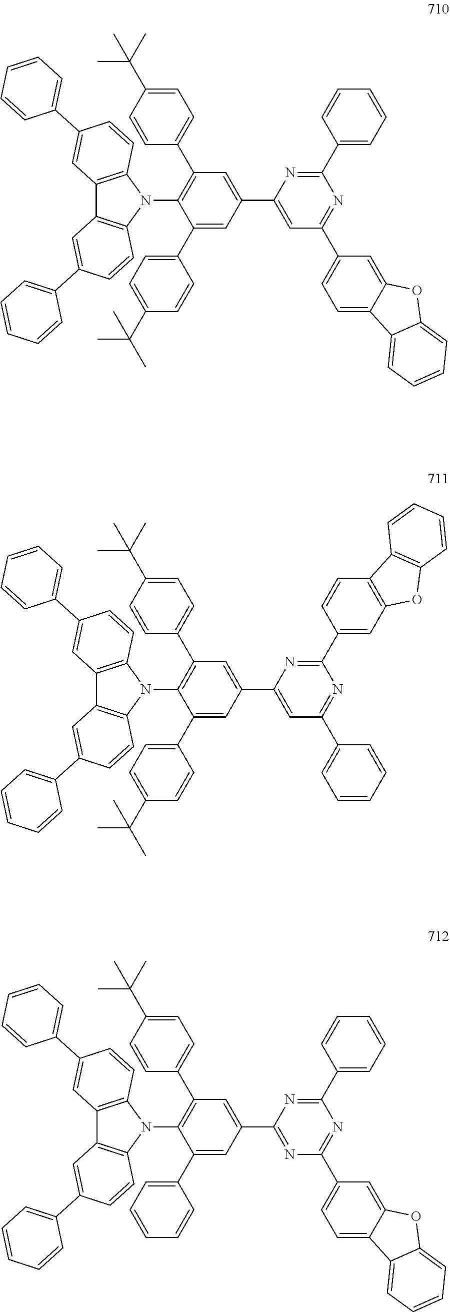 [SMILES: CC(C)(C)c1ccc(-c2cc(-c3cc(-c4ccc5c(c4)oc4ccccc45)nc(-c4ccccc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3cc(-c4ccccc4)nc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)nc(-c4ccc5c(c4)oc4ccccc45)n3)cc(-c3ccccc3)c2-n2c3ccc(-c4ccccc4)cc3c3cc(-c4ccccc4)ccc32)cc1]

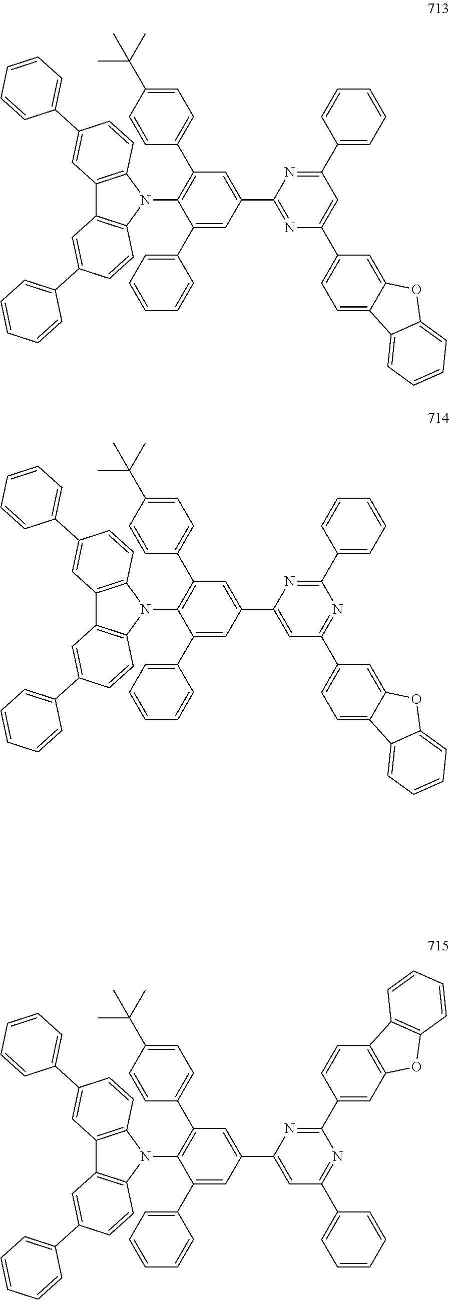 [SMILES: CC(C)(C)c1ccc(-c2cc(-c3nc(-c4ccccc4)cc(-c4ccc5c(c4)oc4ccccc45)n3)cc(-c3ccccc3)c2-n2c3ccc(-c4ccccc4)cc3c3cc(-c4ccccc4)ccc32)cc1 ; CC(C)(C)c1ccc(-c2cc(-c3cc(-c4ccc5c(c4)oc4ccccc45)nc(-c4ccccc4)n3)cc(-c3ccccc3)c2-n2c3ccc(-c4ccccc4)cc3c3cc(-c4ccccc4)ccc32)cc1 ; CC(C)(C)c1ccc(-c2cc(-c3cc(-c4ccccc4)nc(-c4ccc5c(c4)oc4ccccc45)n3)cc(-c3ccccc3)c2-n2c3ccc(-c4ccccc4)cc3c3cc(-c4ccccc4)ccc32)cc1]

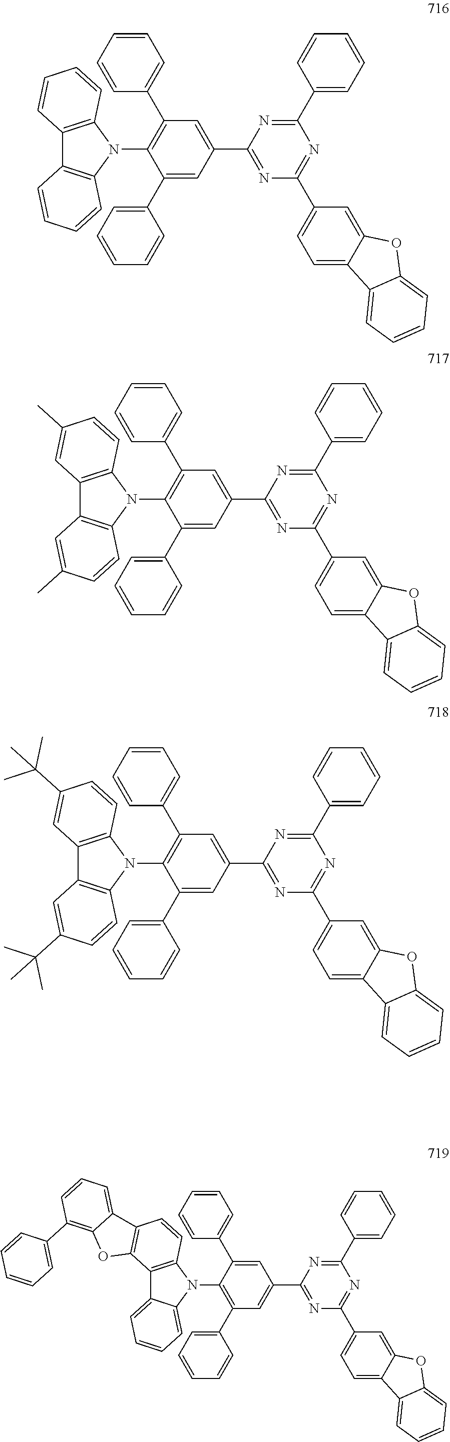 [SMILES: c1ccc(-c2nc(-c3cc(-c4ccccc4)c(-n4c5ccccc5c5ccccc54)c(-c4ccccc4)c3)nc(-c3ccc4c(c3)oc3ccccc34)n2)cc1 ; Cc1ccc2c(c1)c1cc(C)ccc1n2-c1c(-c2ccccc2)cc(-c2nc(-c3ccccc3)nc(-c3ccc4c(c3)oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccccc3)nc(-c3ccc4c(c3)oc3ccccc34)n2)cc1-c1ccccc1 ; c1ccc(-c2nc(-c3cc(-c4ccccc4)c(-n4c5ccccc5c5c6oc7c(-c8ccccc8)cccc7c6ccc54)c(-c4ccccc4)c3)nc(-c3ccc4c(c3)oc3ccccc34)n2)cc1]

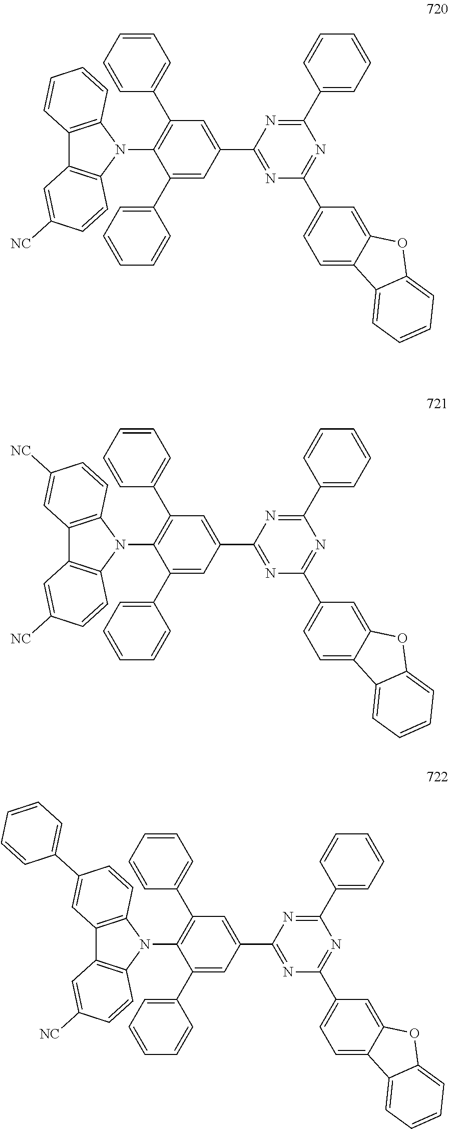 [SMILES: N#Cc1ccc2c(c1)c1ccccc1n2-c1c(-c2ccccc2)cc(-c2nc(-c3ccccc3)nc(-c3ccc4c(c3)oc3ccccc34)n2)cc1-c1ccccc1 ; N#Cc1ccc2c(c1)c1cc(C#N)ccc1n2-c1c(-c2ccccc2)cc(-c2nc(-c3ccccc3)nc(-c3ccc4c(c3)oc3ccccc34)n2)cc1-c1ccccc1 ; N#Cc1ccc2c(c1)c1cc(-c3ccccc3)ccc1n2-c1c(-c2ccccc2)cc(-c2nc(-c3ccccc3)nc(-c3ccc4c(c3)oc3ccccc34)n2)cc1-c1ccccc1]

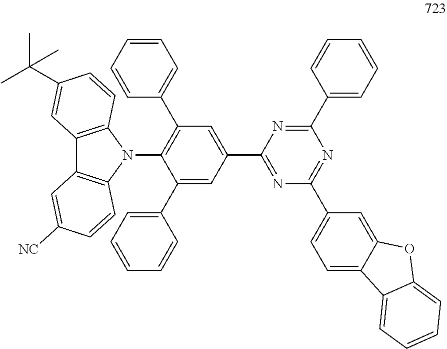 [SMILES: CC(C)(C)c1ccc2c(c1)c1cc(C#N)ccc1n2-c1c(-c2ccccc2)cc(-c2nc(-c3ccccc3)nc(-c3ccc4c(c3)oc3ccccc34)n2)cc1-c1ccccc1]

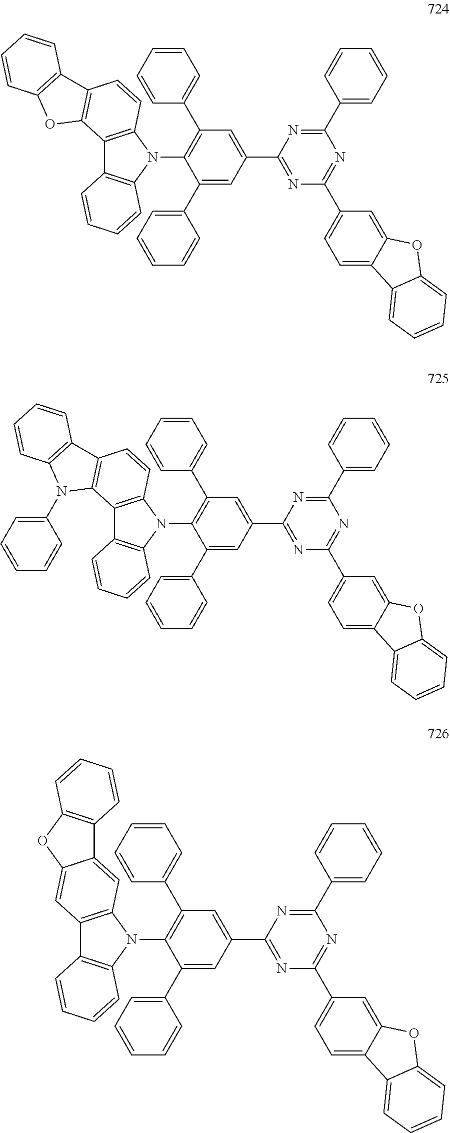 [SMILES: c1ccc(-c2nc(-c3cc(-c4ccccc4)c(-n4c5ccccc5c5c6oc7ccccc7c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6oc7ccccc7c6cc54)c(-c4ccccc4)c3)nc(-c3ccc4c(c3)oc3ccccc34)n2)cc1]

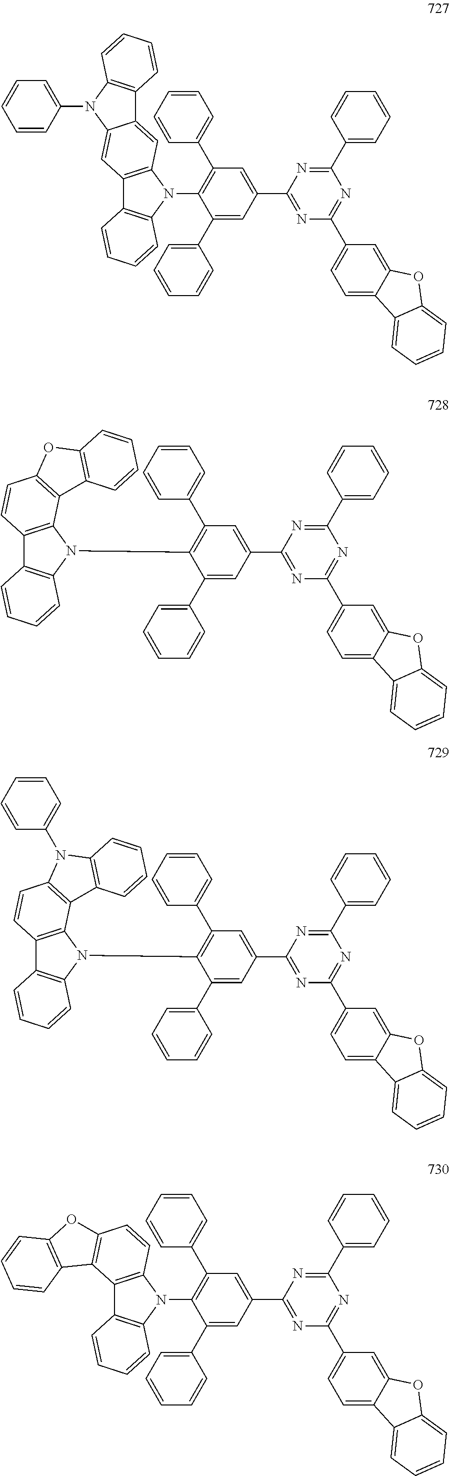 [SMILES: c1ccc(-c2nc(-c3cc(-c4ccccc4)c(-n4c5ccccc5c5cc6c(cc54)c4ccccc4n6-c4ccccc4)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6oc7ccccc7c6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6c(c7ccccc7n6-c6ccccc6)c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)oc4ccccc46)c(-c4ccccc4)c3)nc(-c3ccc4c(c3)oc3ccccc34)n2)cc1]

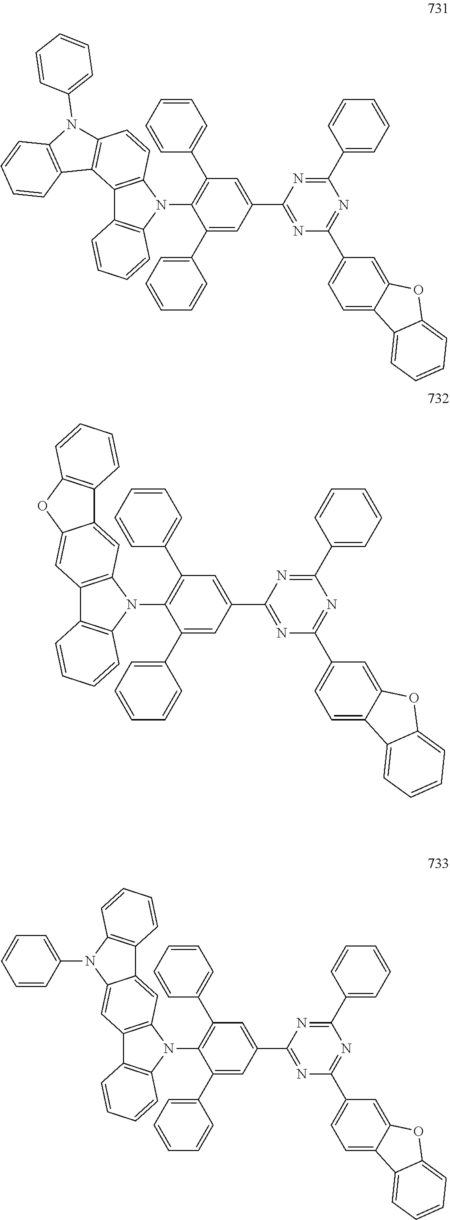 [SMILES: c1ccc(-c2nc(-c3cc(-c4ccccc4)c(-n4c5ccccc5c5c6c7ccccc7n(-c7ccccc7)c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6oc7ccccc7c6cc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)nc(-c3ccc4c(c3)oc3ccccc34)n2)cc1]

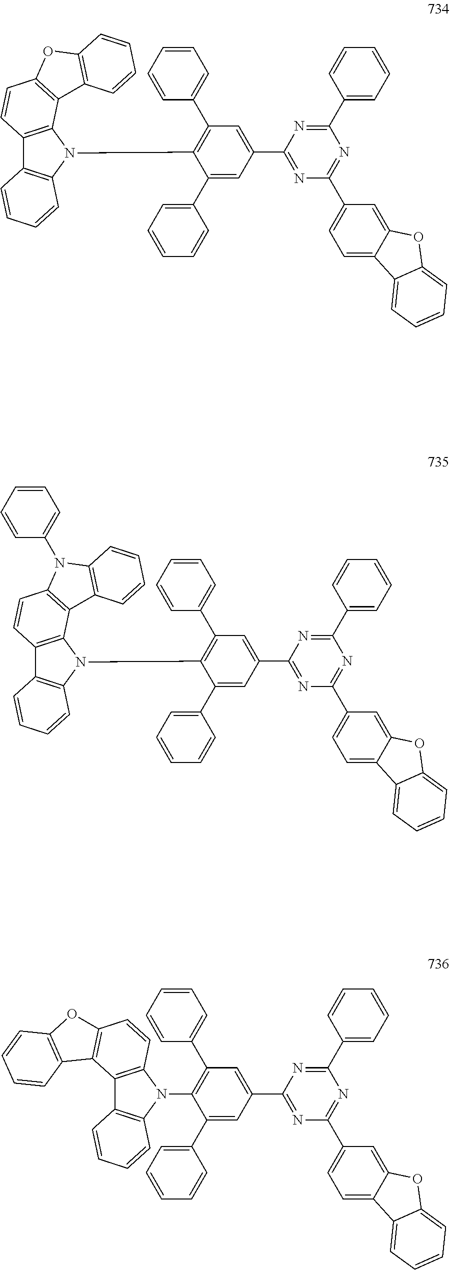 [SMILES: c1ccc(-c2nc(-c3cc(-c4ccccc4)c(-n4c5ccccc5c5ccc6oc7ccccc7c6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6c(c7ccccc7n6-c6ccccc6)c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)oc4ccccc46)c(-c4ccccc4)c3)nc(-c3ccc4c(c3)oc3ccccc34)n2)cc1]

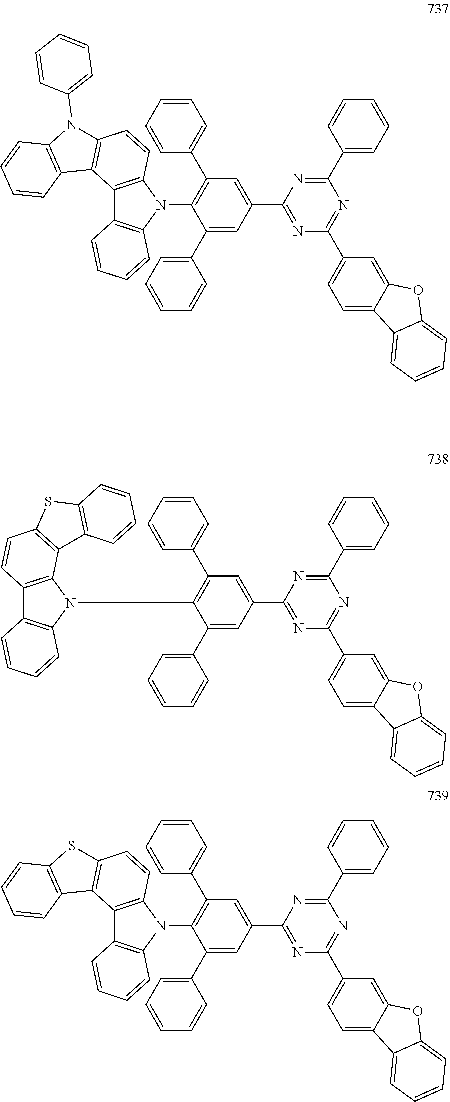 [SMILES: c1ccc(-c2nc(-c3cc(-c4ccccc4)c(-n4c5ccccc5c5c6c7ccccc7n(-c7ccccc7)c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6sc7ccccc7c6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)sc4ccccc46)c(-c4ccccc4)c3)nc(-c3ccc4c(c3)oc3ccccc34)n2)cc1]

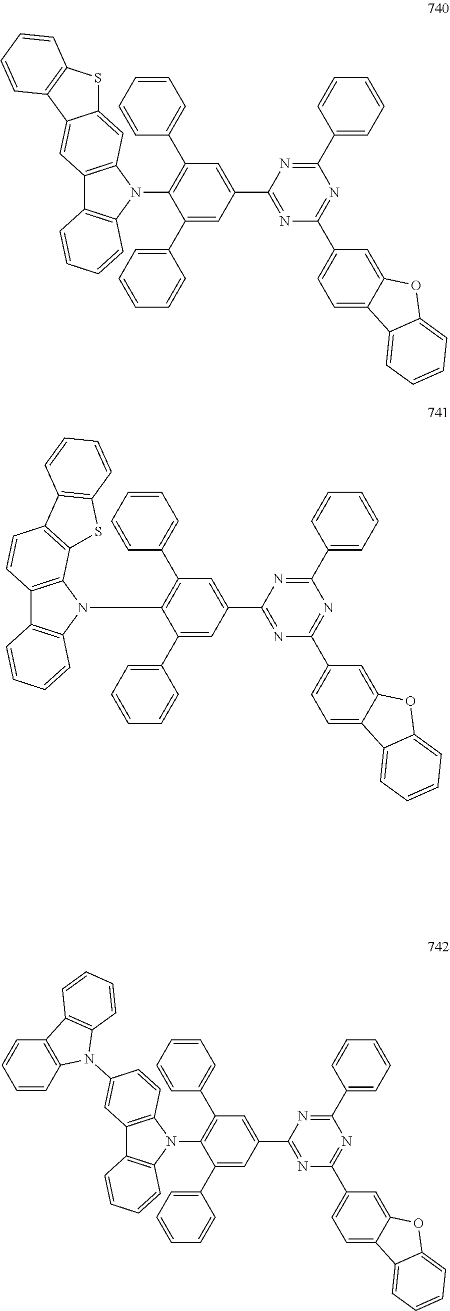 [SMILES: c1ccc(-c2nc(-c3cc(-c4ccccc4)c(-n4c5ccccc5c5cc6c(cc54)sc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6c7ccccc7sc6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc(-n6c7ccccc7c7ccccc76)ccc54)c(-c4ccccc4)c3)nc(-c3ccc4c(c3)oc3ccccc34)n2)cc1]

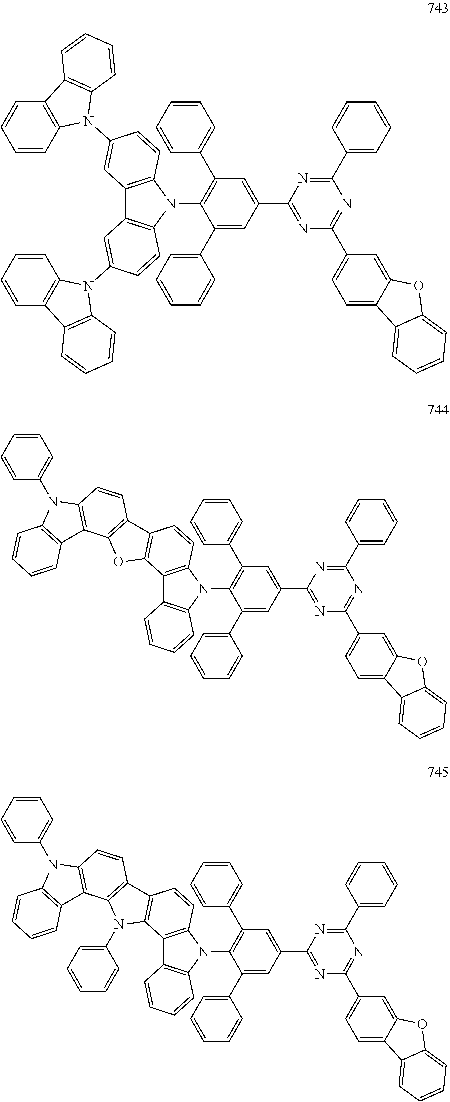 [SMILES: c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)nc(-c3ccc4c(c3)oc3ccccc34)n2)cc1]

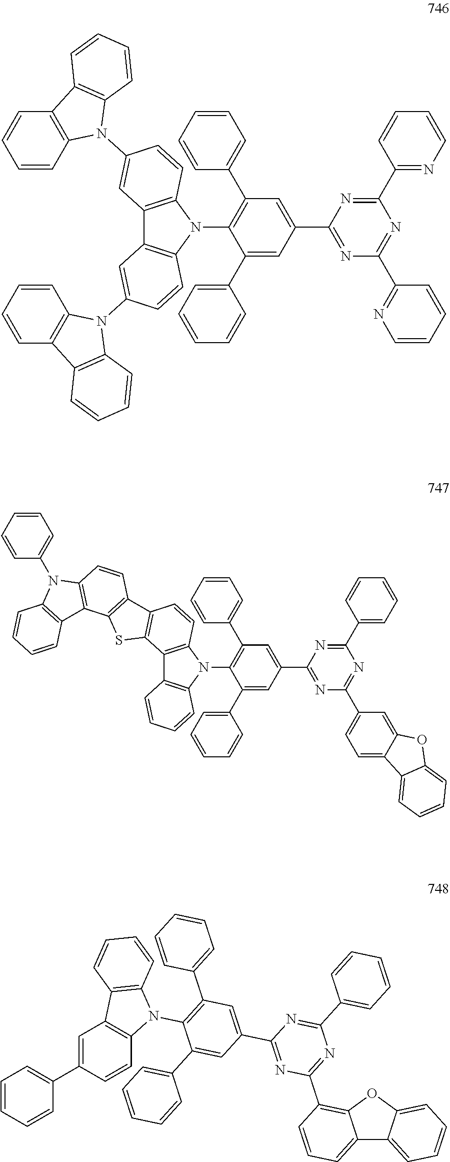 [SMILES: c1ccc(-c2cc(-c3nc(-c4ccccn4)nc(-c4ccccn4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)nc(-c3ccc4c(c3)oc3ccccc34)n2)cc1 ; c1ccc(-c2ccc3c(c2)c2ccccc2n3-c2c(-c3ccccc3)cc(-c3nc(-c4ccccc4)nc(-c4cccc5c4oc4ccccc45)n3)cc2-c2ccccc2)cc1]

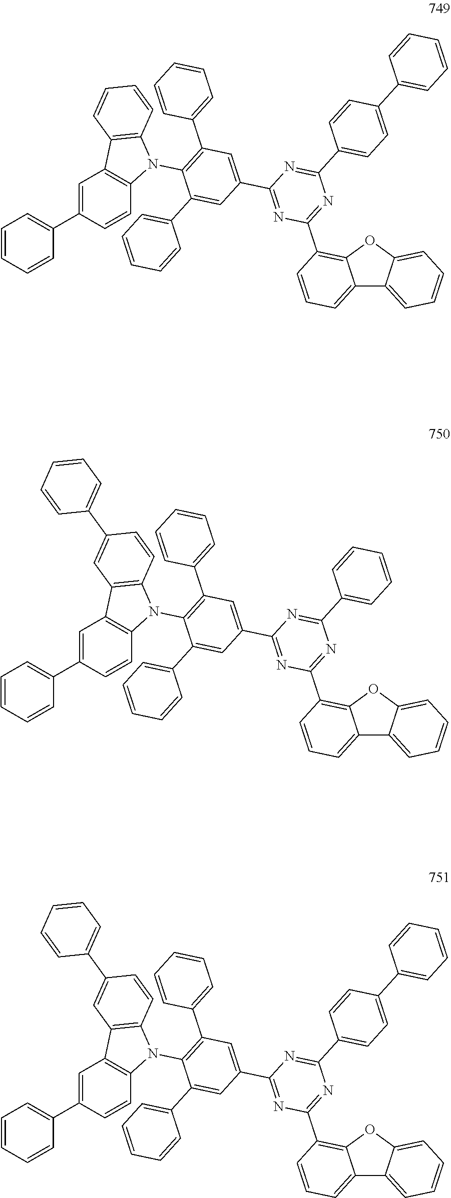 [SMILES: c1ccc(-c2ccc(-c3nc(-c4cc(-c5ccccc5)c(-n5c6ccccc6c6cc(-c7ccccc7)ccc65)c(-c5ccccc5)c4)nc(-c4cccc5c4oc4ccccc45)n3)cc2)cc1 ; c1ccc(-c2ccc3c(c2)c2cc(-c4ccccc4)ccc2n3-c2c(-c3ccccc3)cc(-c3nc(-c4ccccc4)nc(-c4cccc5c4oc4ccccc45)n3)cc2-c2ccccc2)cc1 ; c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5c4oc4ccccc45)n3)cc2)cc1]

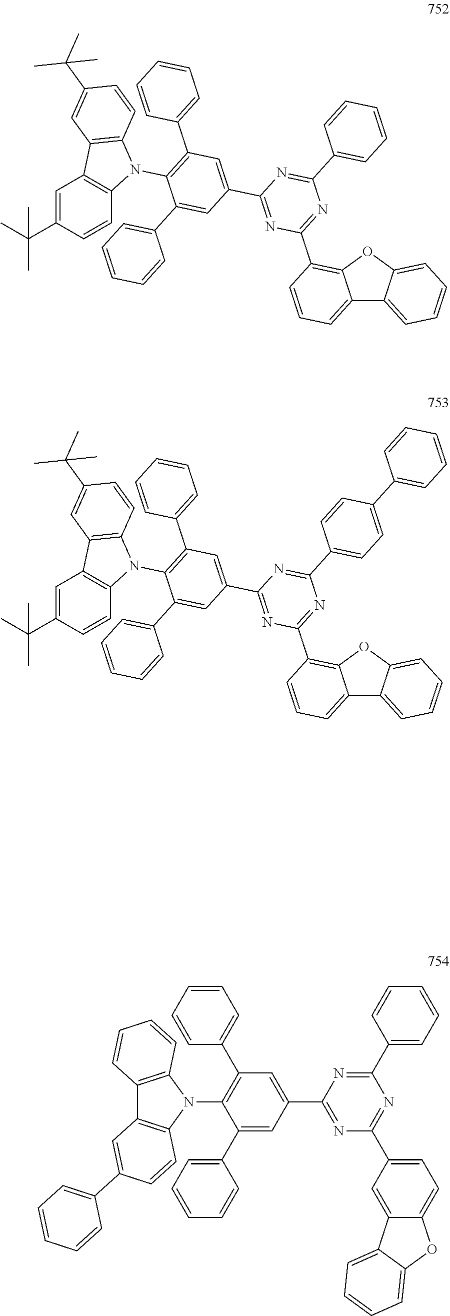 [SMILES: CC(C)(C)c1ccc2c(c1)c1cc(C(C)(C)C)ccc1n2-c1c(-c2ccccc2)cc(-c2nc(-c3ccccc3)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4c3oc3ccccc34)n2)cc1-c1ccccc1 ; c1ccc(-c2ccc3c(c2)c2ccccc2n3-c2c(-c3ccccc3)cc(-c3nc(-c4ccccc4)nc(-c4ccc5oc6ccccc6c5c4)n3)cc2-c2ccccc2)cc1]

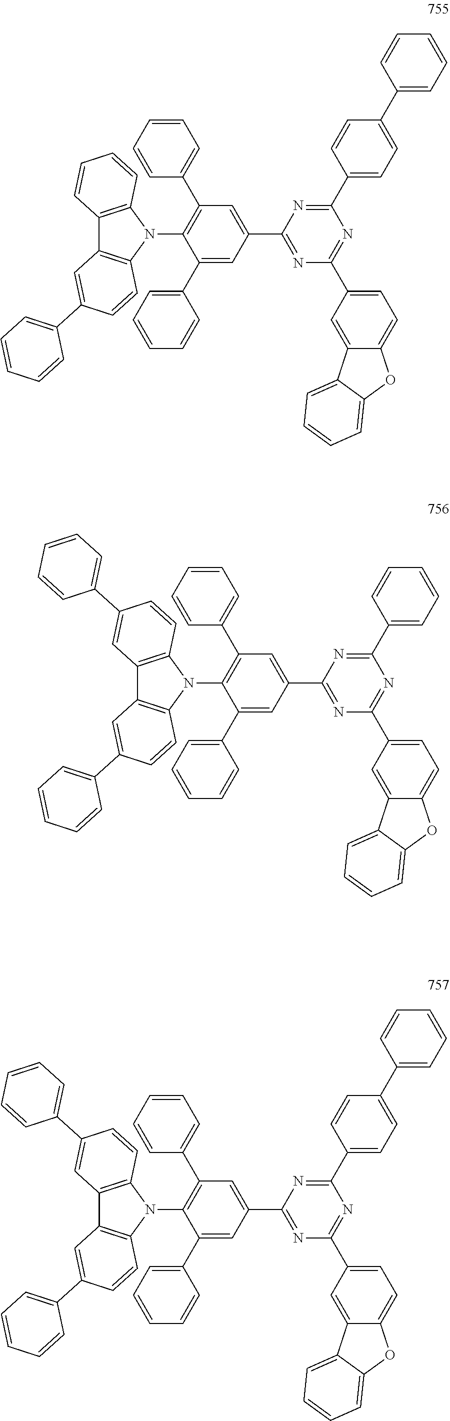 [SMILES: c1ccc(-c2ccc(-c3nc(-c4cc(-c5ccccc5)c(-n5c6ccccc6c6cc(-c7ccccc7)ccc65)c(-c5ccccc5)c4)nc(-c4ccc5oc6ccccc6c5c4)n3)cc2)cc1 ; c1ccc(-c2ccc3c(c2)c2cc(-c4ccccc4)ccc2n3-c2c(-c3ccccc3)cc(-c3nc(-c4ccccc4)nc(-c4ccc5oc6ccccc6c5c4)n3)cc2-c2ccccc2)cc1 ; c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4ccc5oc6ccccc6c5c4)n3)cc2)cc1]

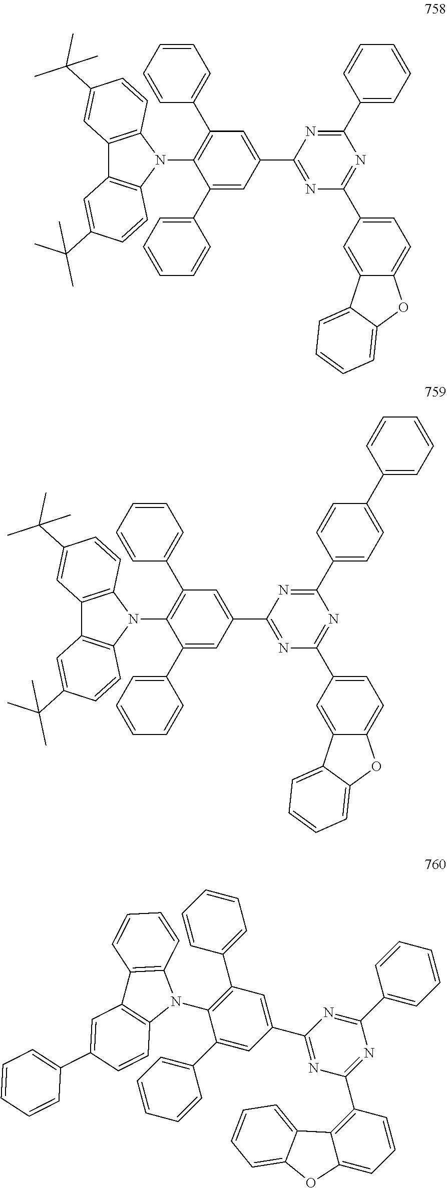 [SMILES: CC(C)(C)c1ccc2c(c1)c1cc(C(C)(C)C)ccc1n2-c1c(-c2ccccc2)cc(-c2nc(-c3ccccc3)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3ccc4oc5ccccc5c4c3)n2)cc1-c1ccccc1 ; c1ccc(-c2ccc3c(c2)c2ccccc2n3-c2c(-c3ccccc3)cc(-c3nc(-c4ccccc4)nc(-c4cccc5oc6ccccc6c45)n3)cc2-c2ccccc2)cc1]

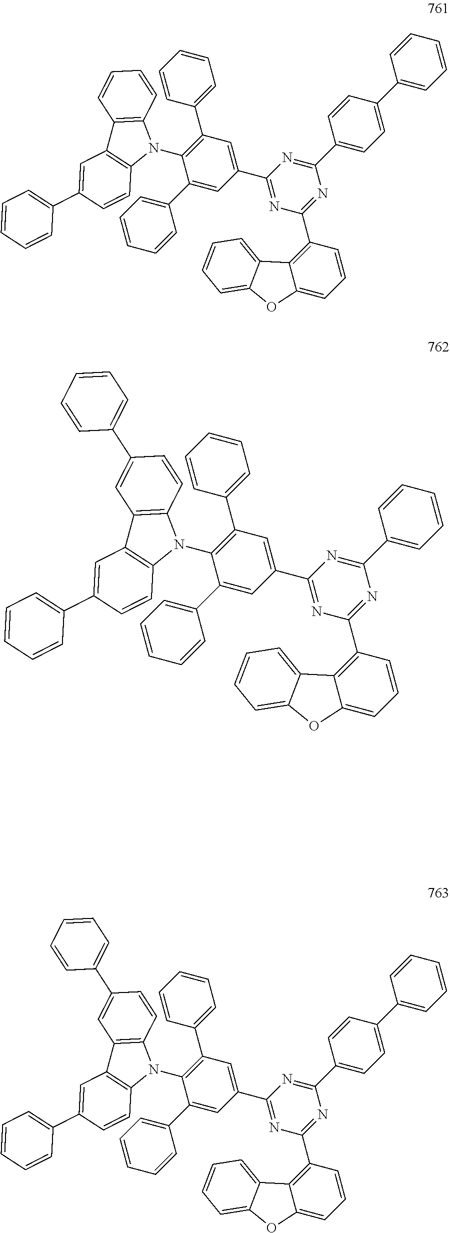 [SMILES: c1ccc(-c2ccc(-c3nc(-c4cc(-c5ccccc5)c(-n5c6ccccc6c6cc(-c7ccccc7)ccc65)c(-c5ccccc5)c4)nc(-c4cccc5oc6ccccc6c45)n3)cc2)cc1 ; c1ccc(-c2ccc3c(c2)c2cc(-c4ccccc4)ccc2n3-c2c(-c3ccccc3)cc(-c3nc(-c4ccccc4)nc(-c4cccc5oc6ccccc6c45)n3)cc2-c2ccccc2)cc1 ; c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5oc6ccccc6c45)n3)cc2)cc1]

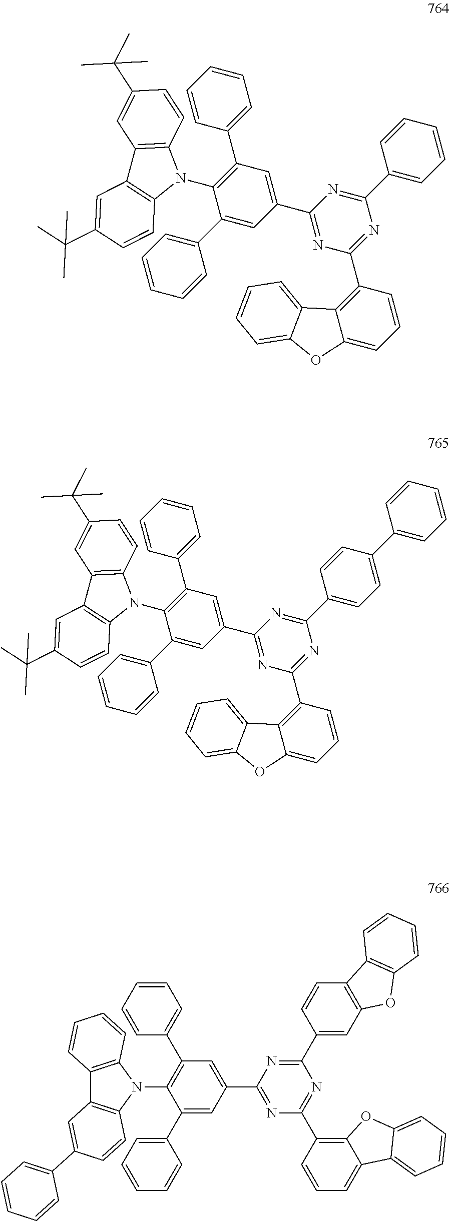 [SMILES: CC(C)(C)c1ccc2c(c1)c1cc(C(C)(C)C)ccc1n2-c1c(-c2ccccc2)cc(-c2nc(-c3ccccc3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4oc5ccccc5c34)n2)cc1-c1ccccc1 ; c1ccc(-c2ccc3c(c2)c2ccccc2n3-c2c(-c3ccccc3)cc(-c3nc(-c4ccc5c(c4)oc4ccccc45)nc(-c4cccc5c4oc4ccccc45)n3)cc2-c2ccccc2)cc1]

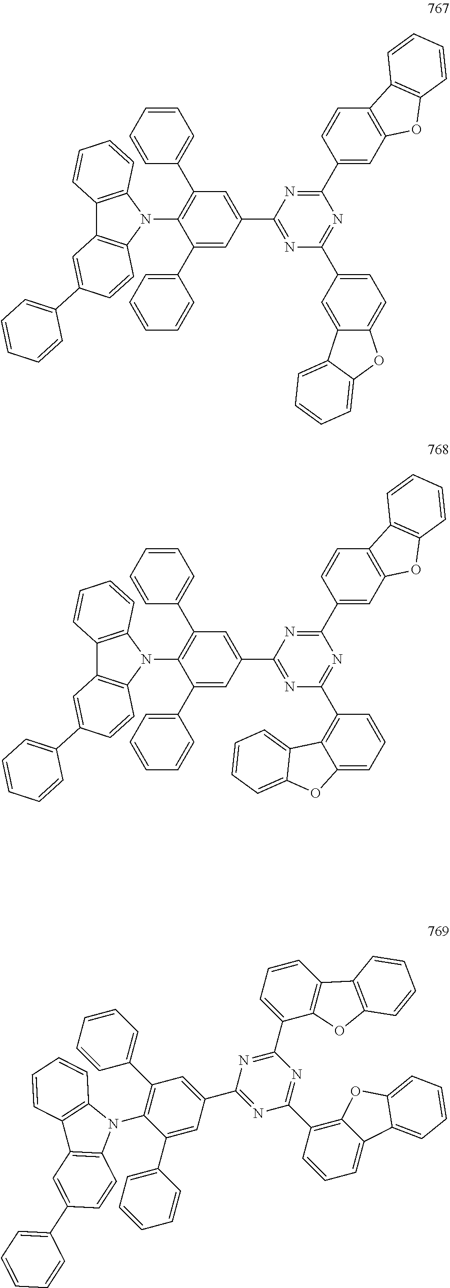 [SMILES: c1ccc(-c2ccc3c(c2)c2ccccc2n3-c2c(-c3ccccc3)cc(-c3nc(-c4ccc5c(c4)oc4ccccc45)nc(-c4ccc5oc6ccccc6c5c4)n3)cc2-c2ccccc2)cc1 ; c1ccc(-c2ccc3c(c2)c2ccccc2n3-c2c(-c3ccccc3)cc(-c3nc(-c4ccc5c(c4)oc4ccccc45)nc(-c4cccc5oc6ccccc6c45)n3)cc2-c2ccccc2)cc1 ; c1ccc(-c2ccc3c(c2)c2ccccc2n3-c2c(-c3ccccc3)cc(-c3nc(-c4cccc5c4oc4ccccc45)nc(-c4cccc5c4oc4ccccc45)n3)cc2-c2ccccc2)cc1]

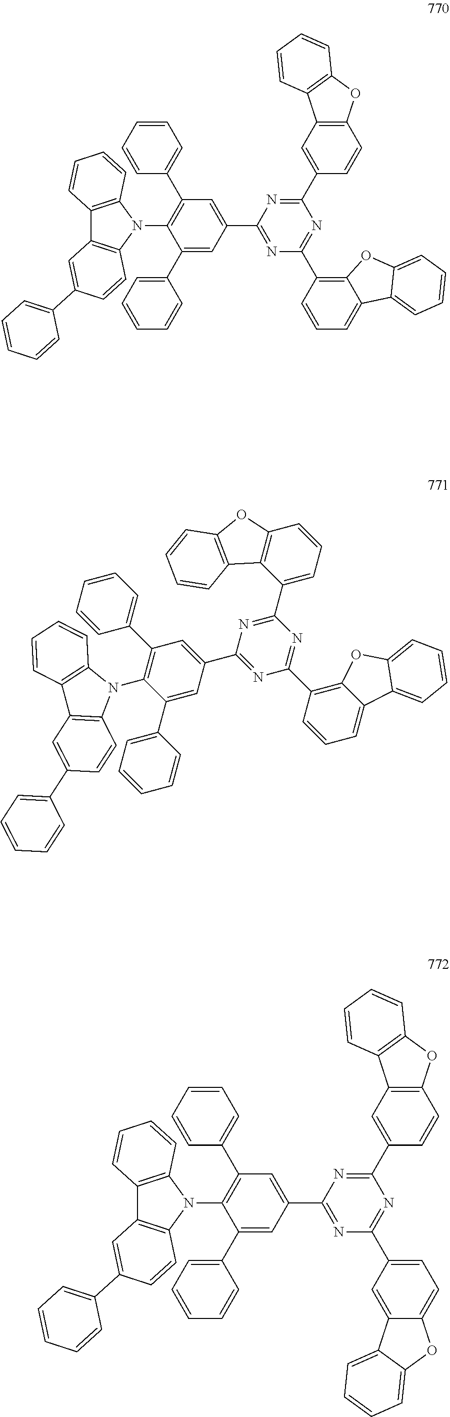 [SMILES: c1ccc(-c2ccc3c(c2)c2ccccc2n3-c2c(-c3ccccc3)cc(-c3nc(-c4ccc5oc6ccccc6c5c4)nc(-c4cccc5c4oc4ccccc45)n3)cc2-c2ccccc2)cc1 ; c1ccc(-c2ccc3c(c2)c2ccccc2n3-c2c(-c3ccccc3)cc(-c3nc(-c4cccc5c4oc4ccccc45)nc(-c4cccc5oc6ccccc6c45)n3)cc2-c2ccccc2)cc1 ; c1ccc(-c2ccc3c(c2)c2ccccc2n3-c2c(-c3ccccc3)cc(-c3nc(-c4ccc5oc6ccccc6c5c4)nc(-c4ccc5oc6ccccc6c5c4)n3)cc2-c2ccccc2)cc1]

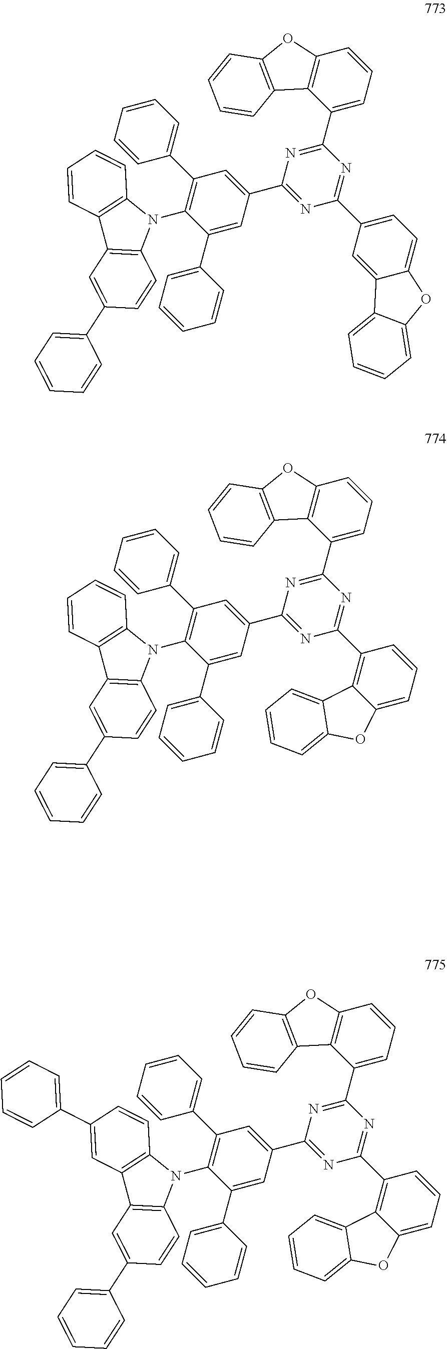 [SMILES: c1ccc(-c2ccc3c(c2)c2ccccc2n3-c2c(-c3ccccc3)cc(-c3nc(-c4ccc5oc6ccccc6c5c4)nc(-c4cccc5oc6ccccc6c45)n3)cc2-c2ccccc2)cc1 ; c1ccc(-c2ccc3c(c2)c2ccccc2n3-c2c(-c3ccccc3)cc(-c3nc(-c4cccc5oc6ccccc6c45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4cccc5oc6ccccc6c45)nc(-c4cccc5oc6ccccc6c45)n3)cc2-c2ccccc2)cc1]

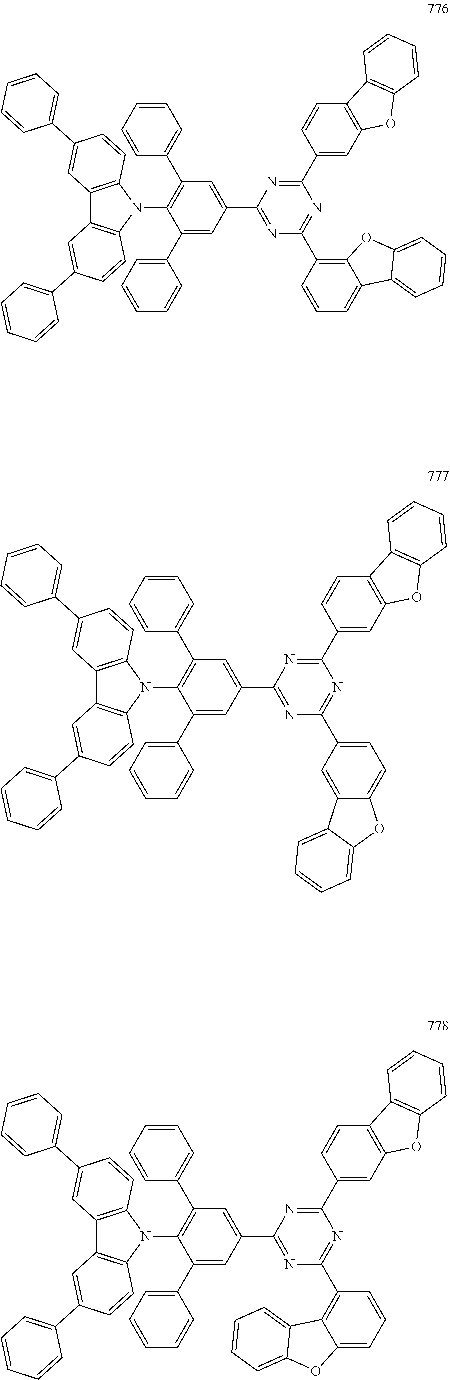 [SMILES: c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5c(c4)oc4ccccc45)nc(-c4ccc5oc6ccccc6c5c4)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5oc6ccccc6c45)n3)cc2-c2ccccc2)cc1]

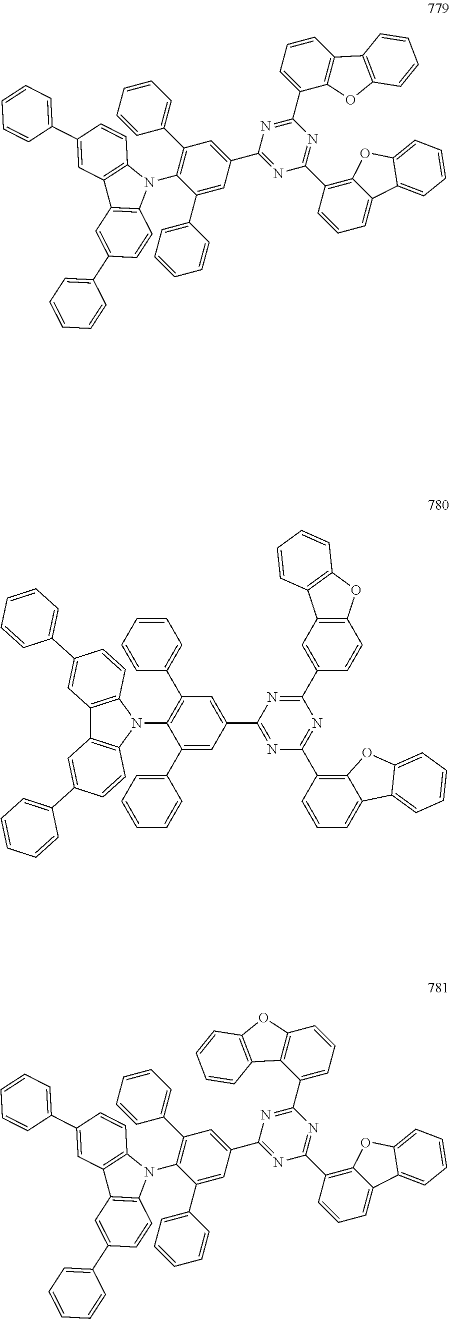 [SMILES: c1ccc(-c2ccc3c(c2)c2cc(-c4ccccc4)ccc2n3-c2c(-c3ccccc3)cc(-c3nc(-c4cccc5c4oc4ccccc45)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4cccc5c4oc4ccccc45)nc(-c4cccc5oc6ccccc6c45)n3)cc2-c2ccccc2)cc1]

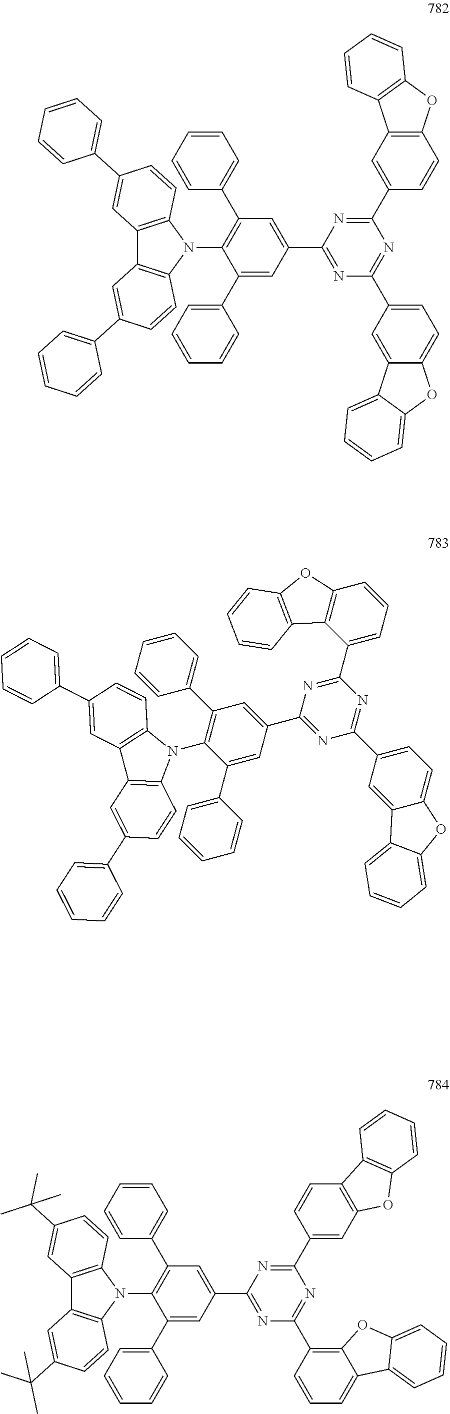 [SMILES: c1ccc(-c2ccc3c(c2)c2cc(-c4ccccc4)ccc2n3-c2c(-c3ccccc3)cc(-c3nc(-c4ccc5oc6ccccc6c5c4)nc(-c4ccc5oc6ccccc6c5c4)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5oc6ccccc6c45)n3)cc2-c2ccccc2)cc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4c3oc3ccccc34)n2)cc1-c1ccccc1]

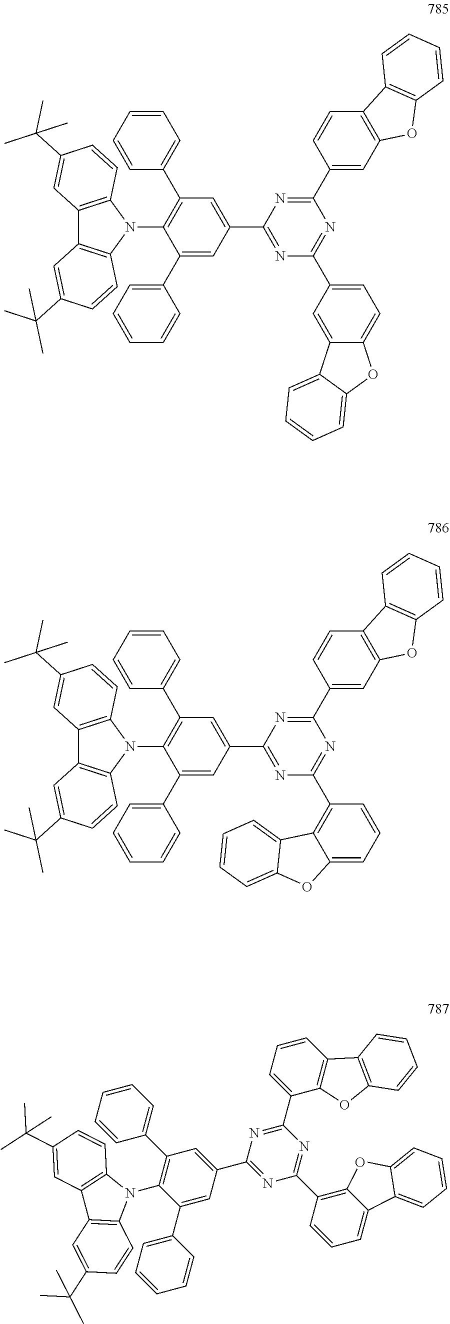 [SMILES: CC(C)(C)c1ccc2c(c1)c1cc(C(C)(C)C)ccc1n2-c1c(-c2ccccc2)cc(-c2nc(-c3ccc4c(c3)oc3ccccc34)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4c3oc3ccccc34)n2)cc1-c1ccccc1]

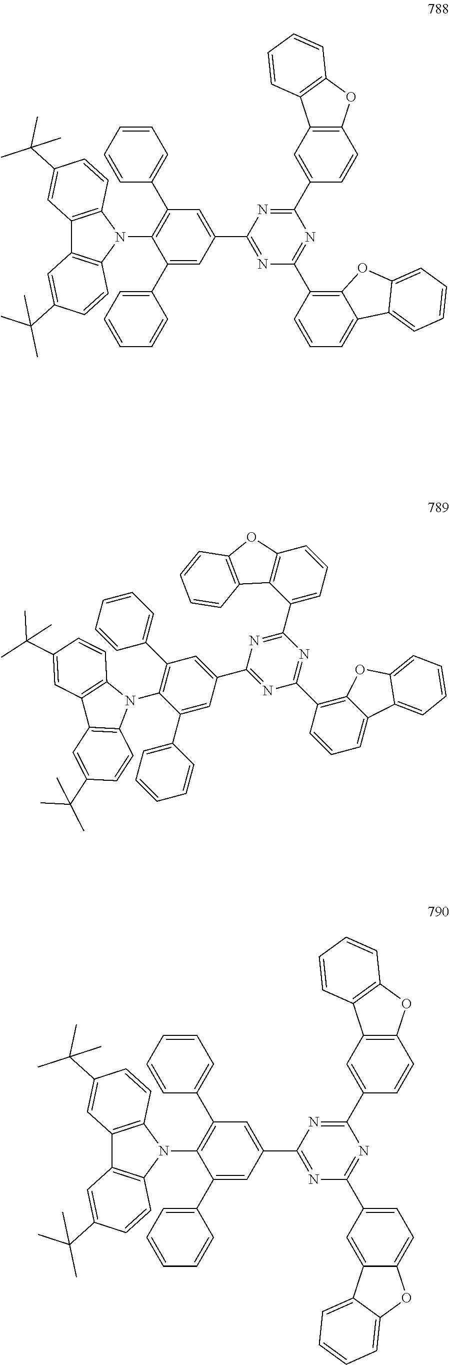 [SMILES: CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3ccc4oc5ccccc5c4c3)nc(-c3ccc4oc5ccccc5c4c3)n2)cc1-c1ccccc1]

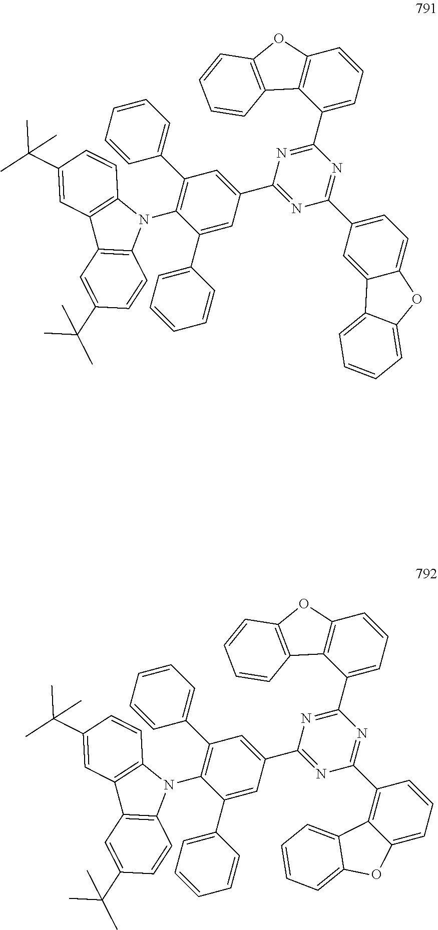 [SMILES: CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3cccc4oc5ccccc5c34)nc(-c3cccc4oc5ccccc5c34)n2)cc1-c1ccccc1]

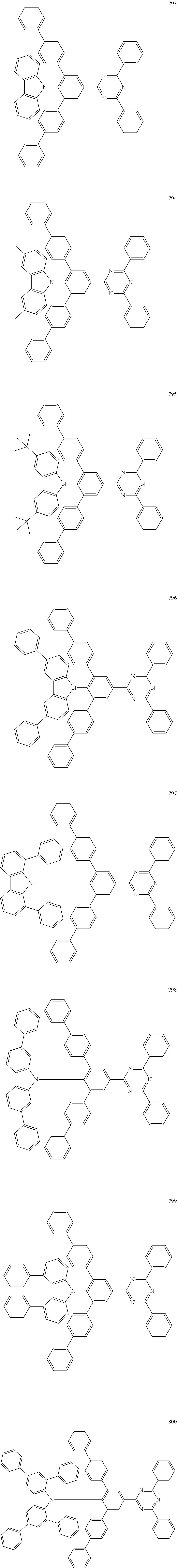

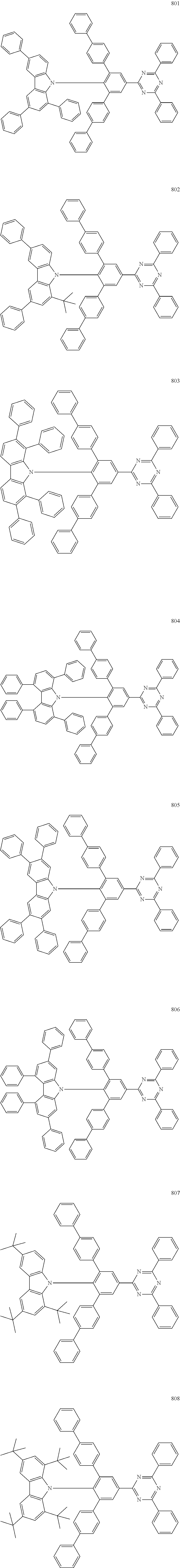

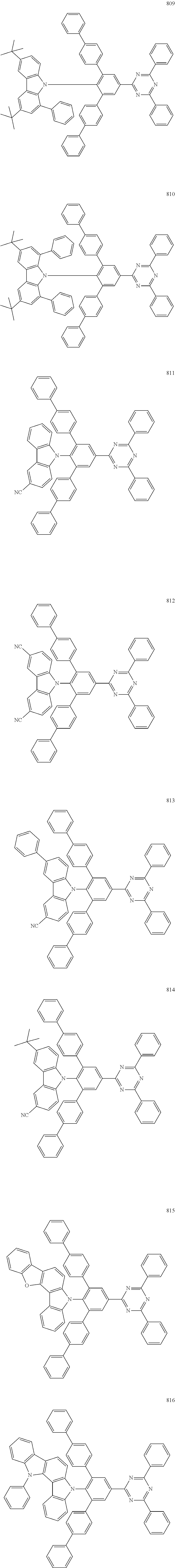

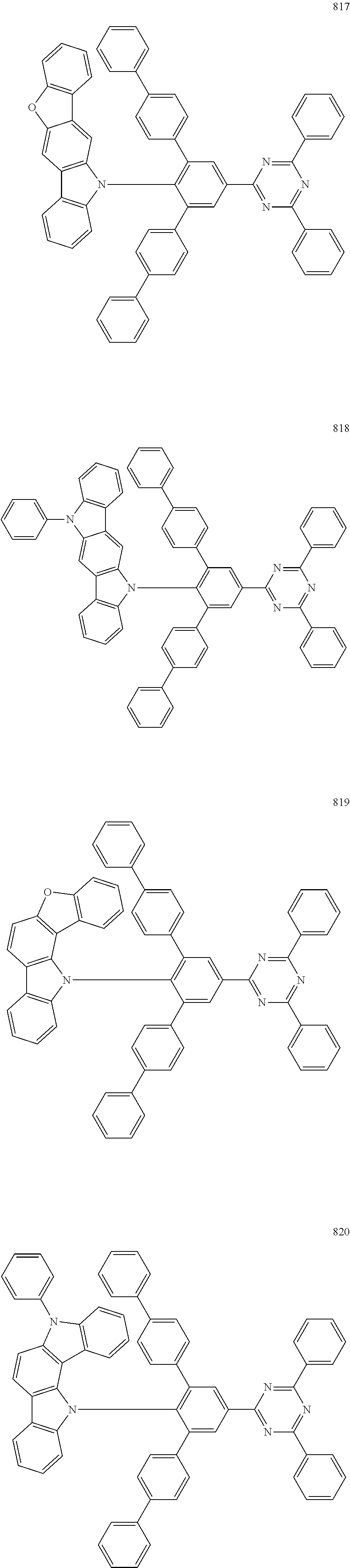 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4cc5oc6ccccc6c5cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc5c(cc43)c3ccccc3n5-c3ccccc3)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5oc6ccccc6c5c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c(c6ccccc6n5-c5ccccc5)c43)cc2)cc1]

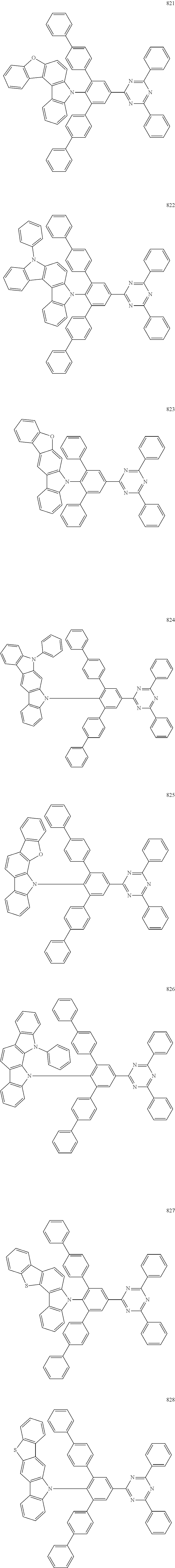

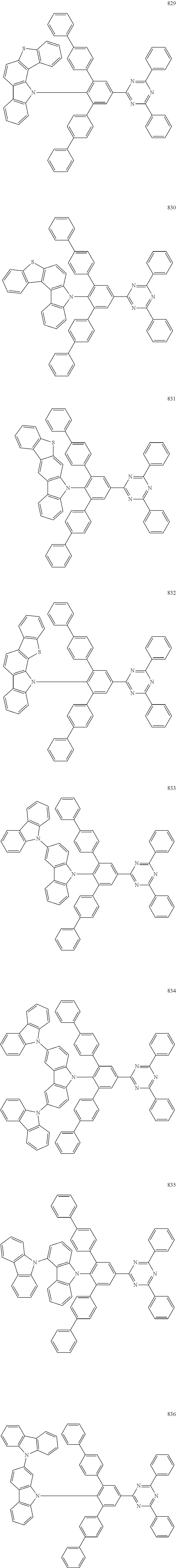

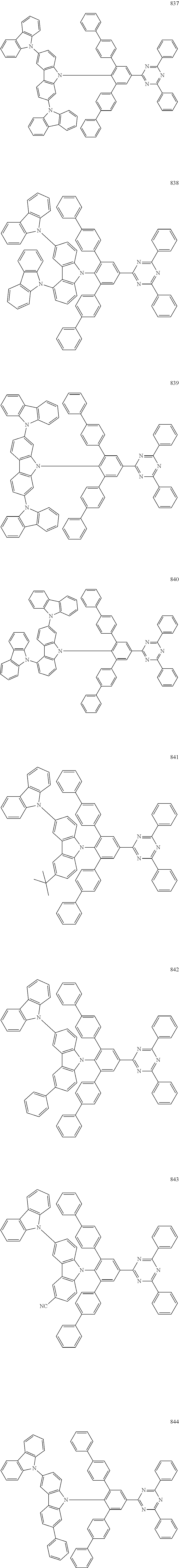

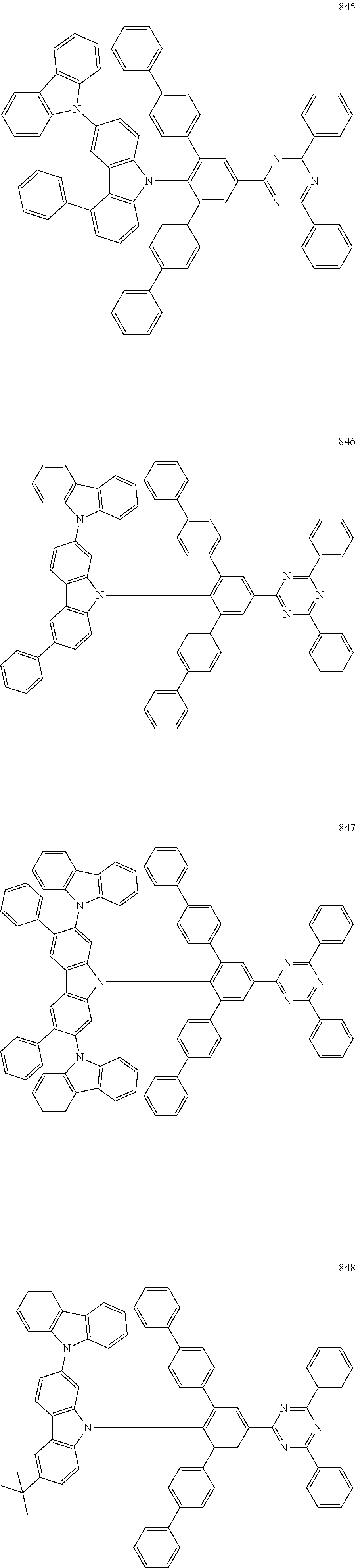

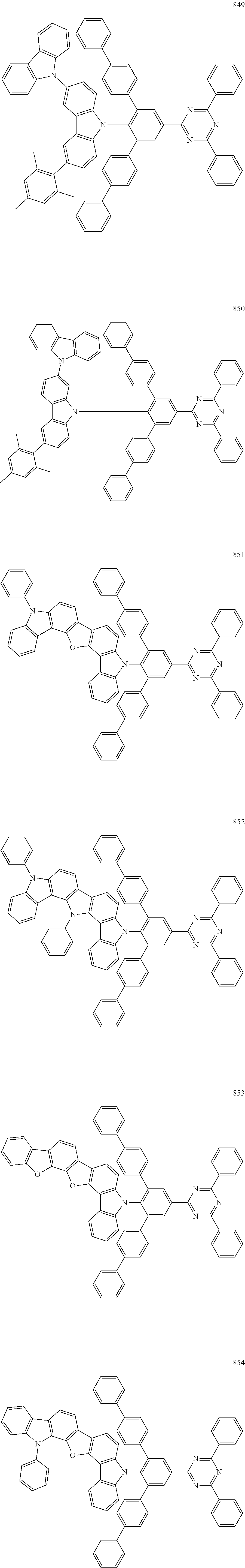

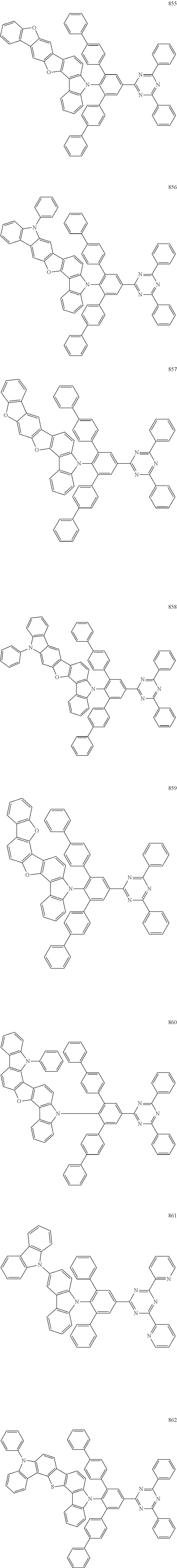

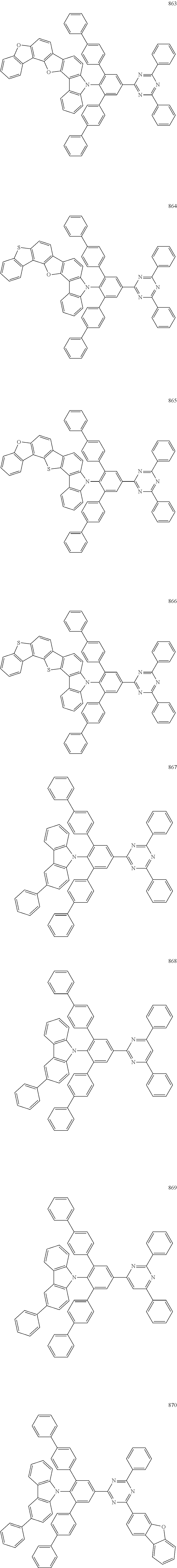

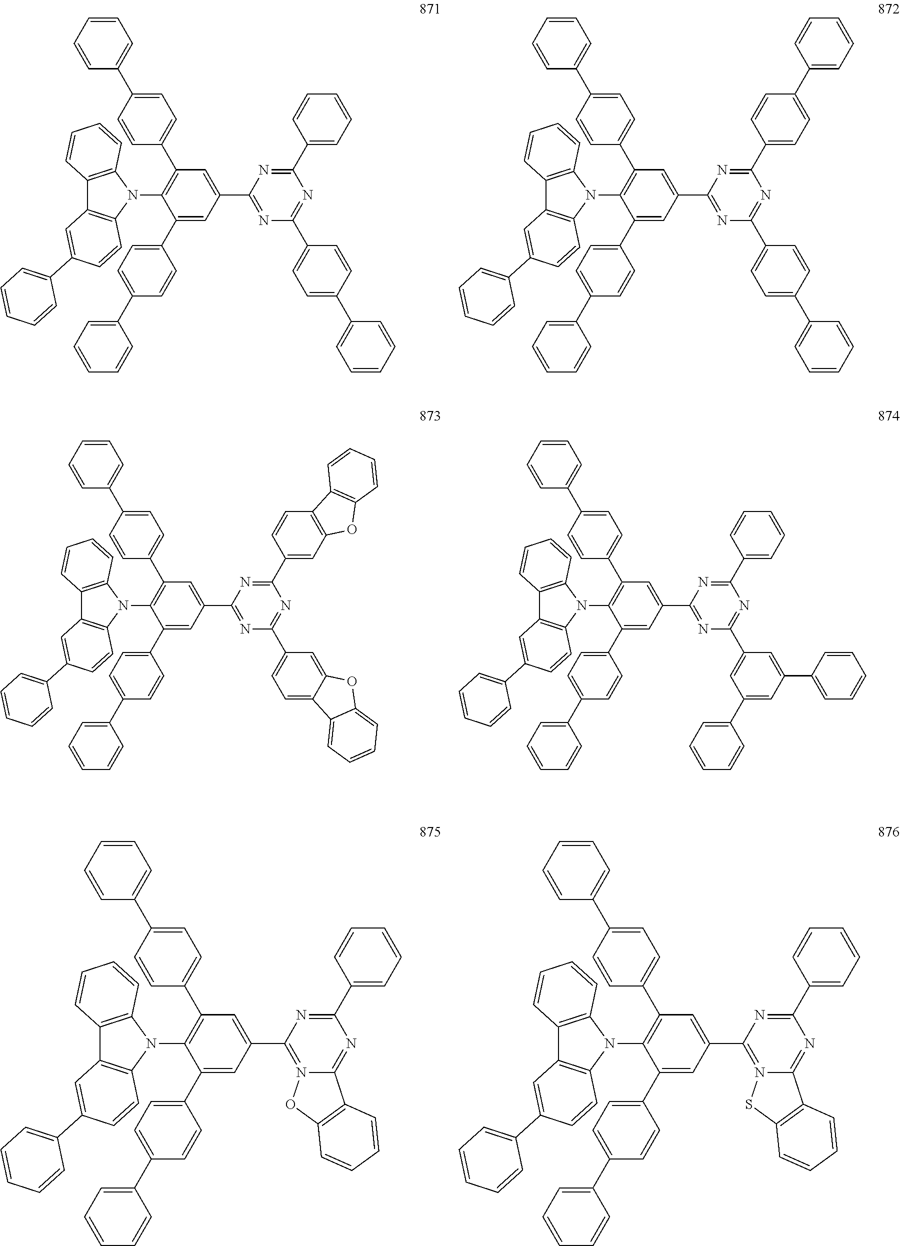

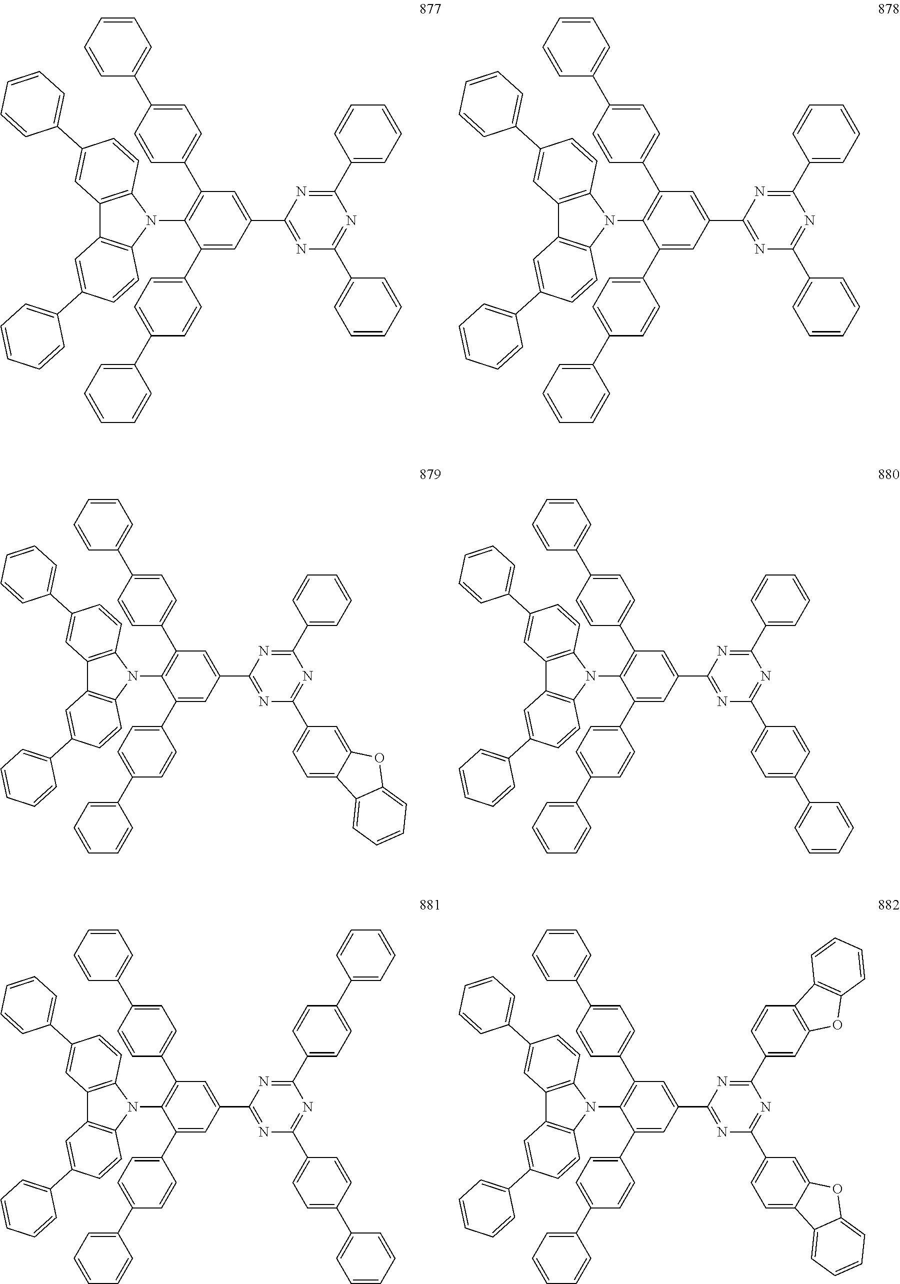

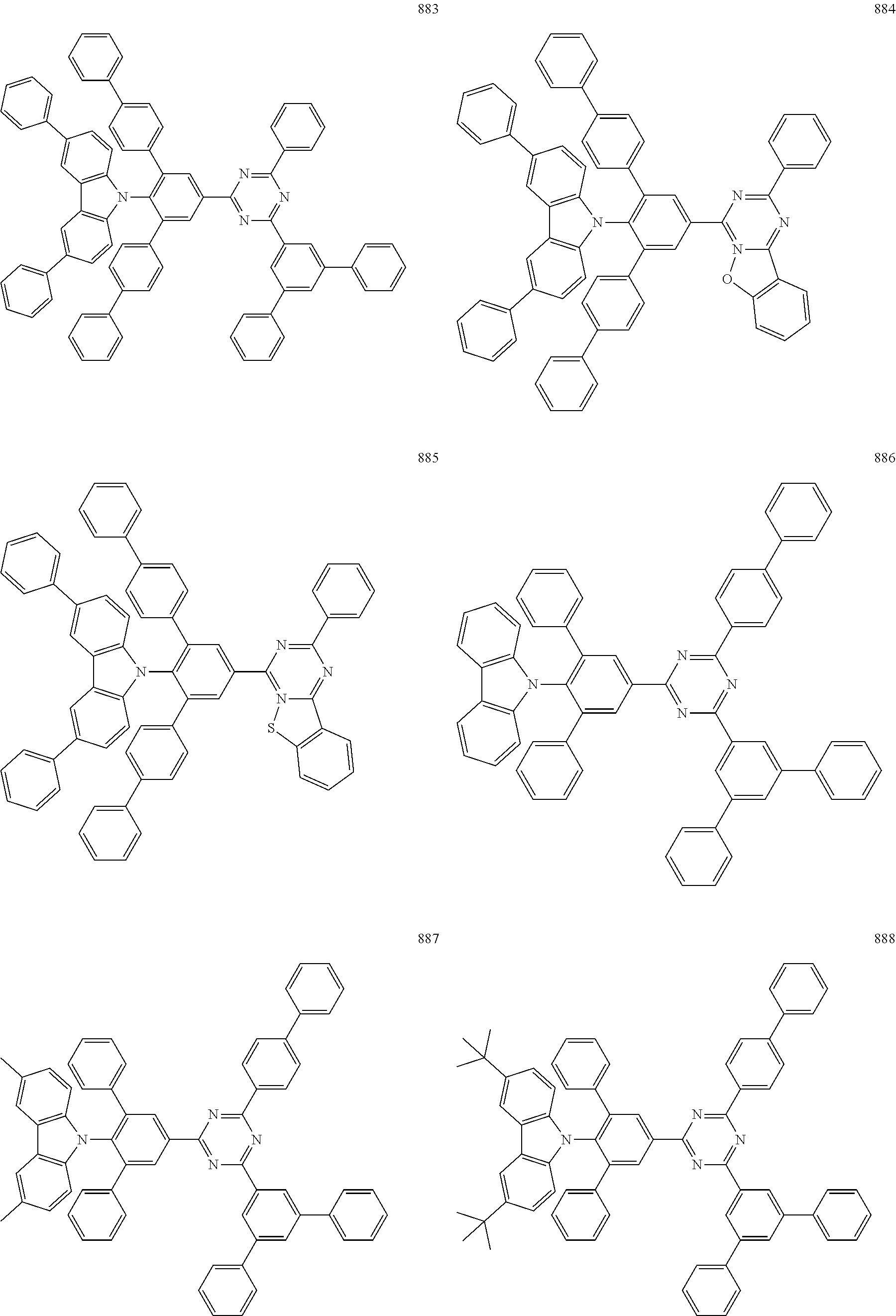

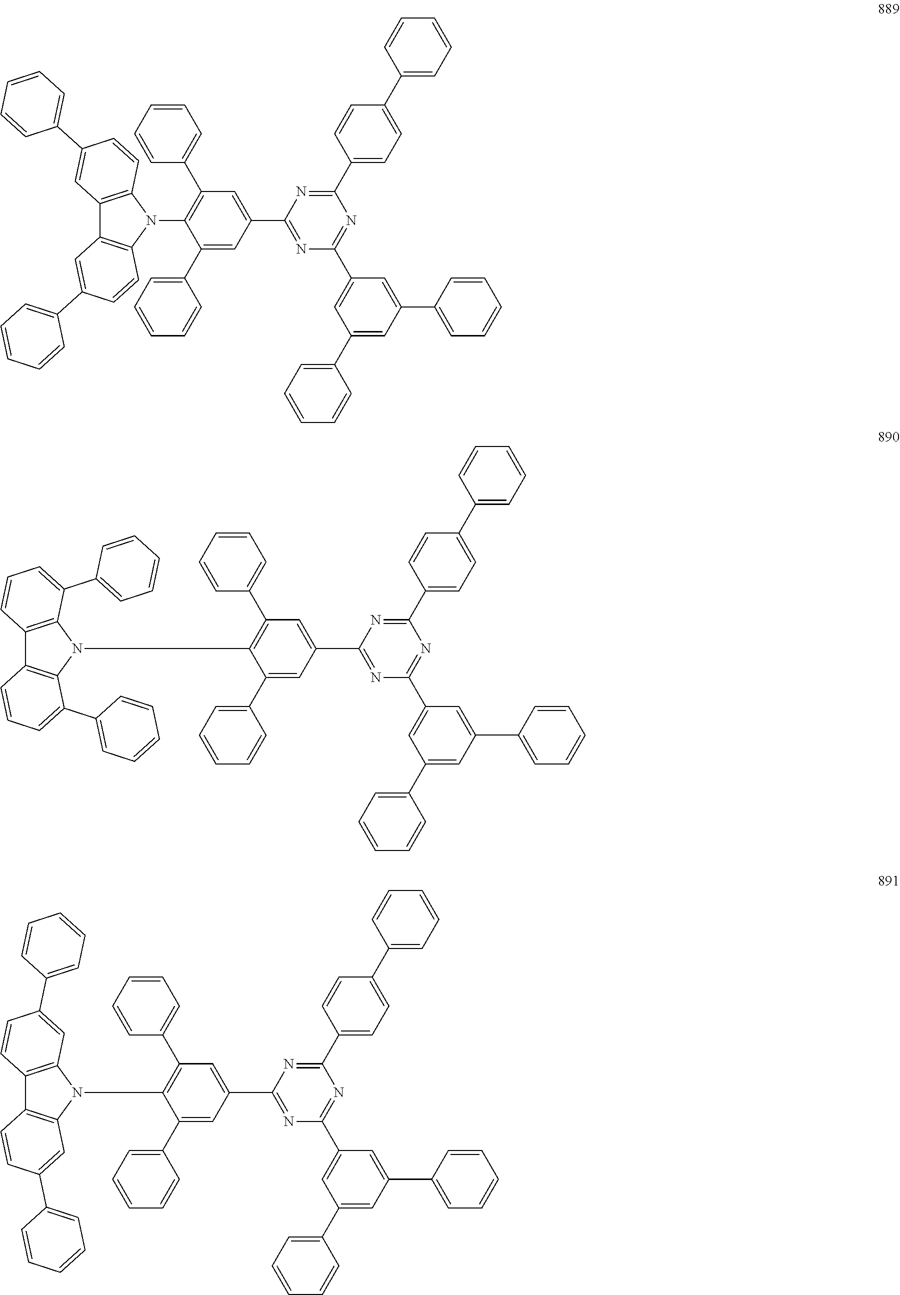

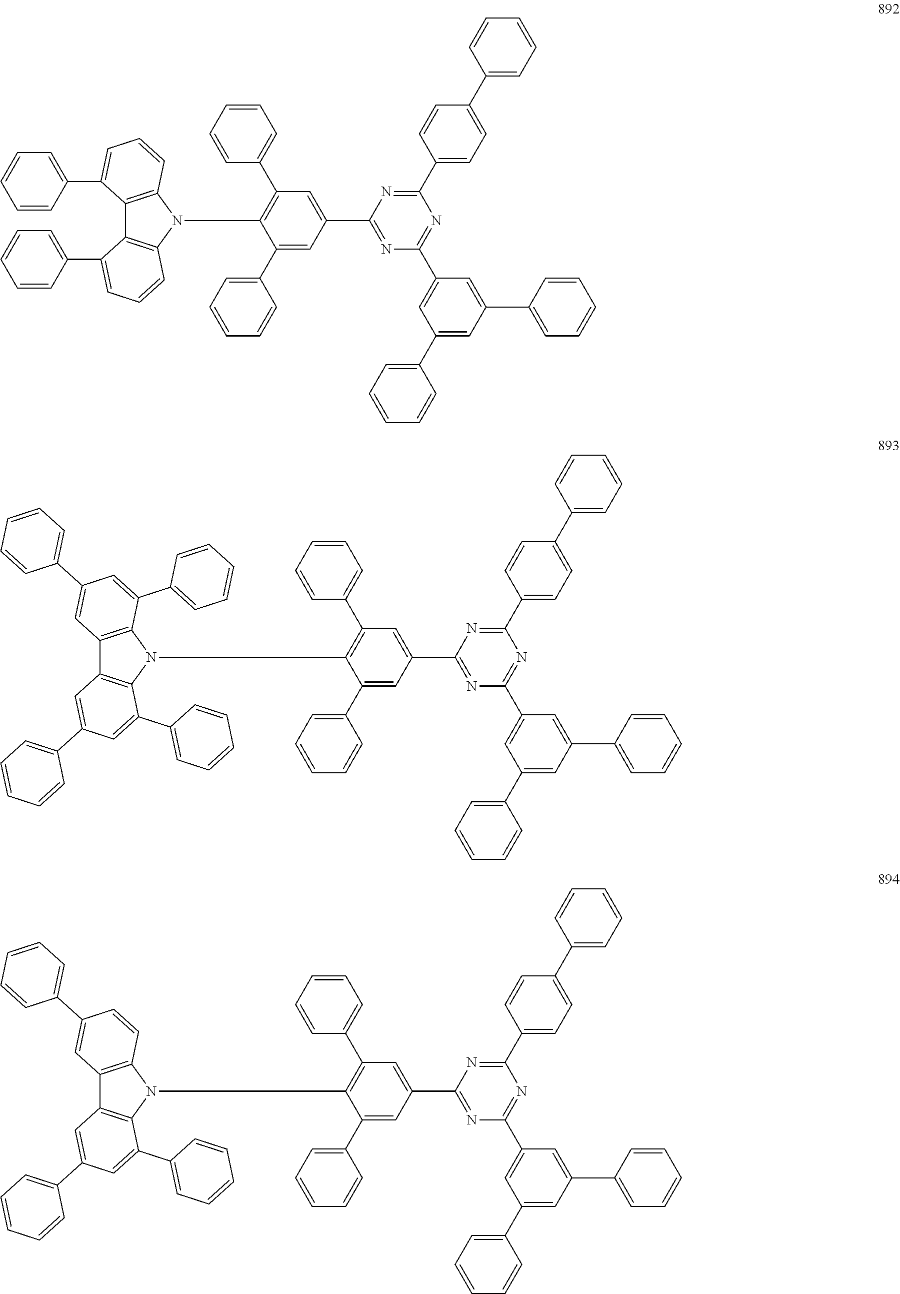 [SMILES: c1ccc(-c2ccc(-c3nc(-c4cc(-c5ccccc5)cc(-c5ccccc5)c4)nc(-c4cc(-c5ccccc5)c(-n5c6cccc(-c7ccccc7)c6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1]

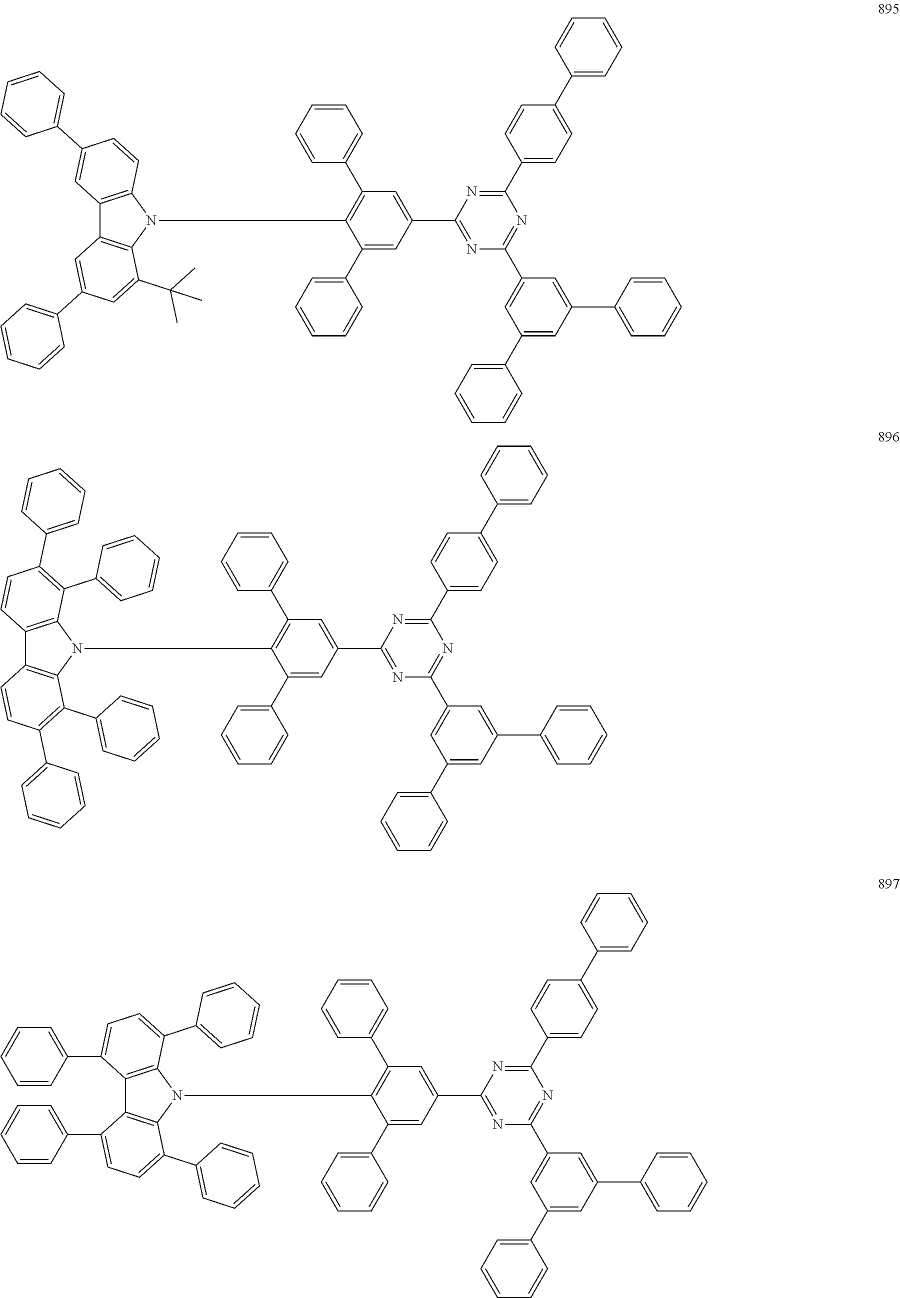

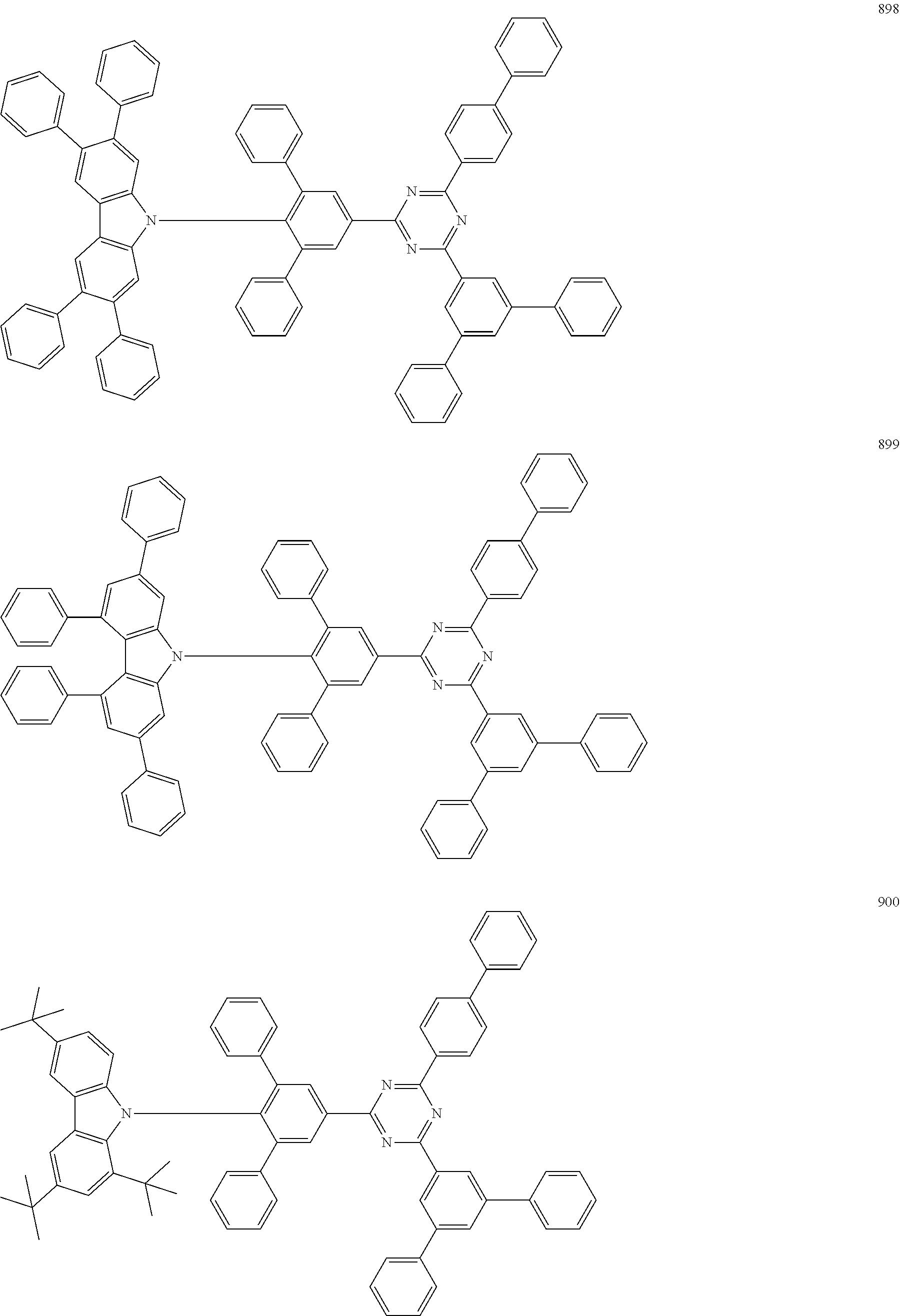 [SMILES: c1ccc(-c2ccc(-c3nc(-c4cc(-c5ccccc5)cc(-c5ccccc5)c4)nc(-c4cc(-c5ccccc5)c(-n5c6cc(-c7ccccc7)c(-c7ccccc7)cc6c6cc(-c7ccccc7)c(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)cc(-c7ccccc7)c6c6c(-c7ccccc7)cc(-c7ccccc7)cc65)c(-c5ccccc5)c4)n3)cc2)cc1 ; CC(C)(C)c1ccc2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

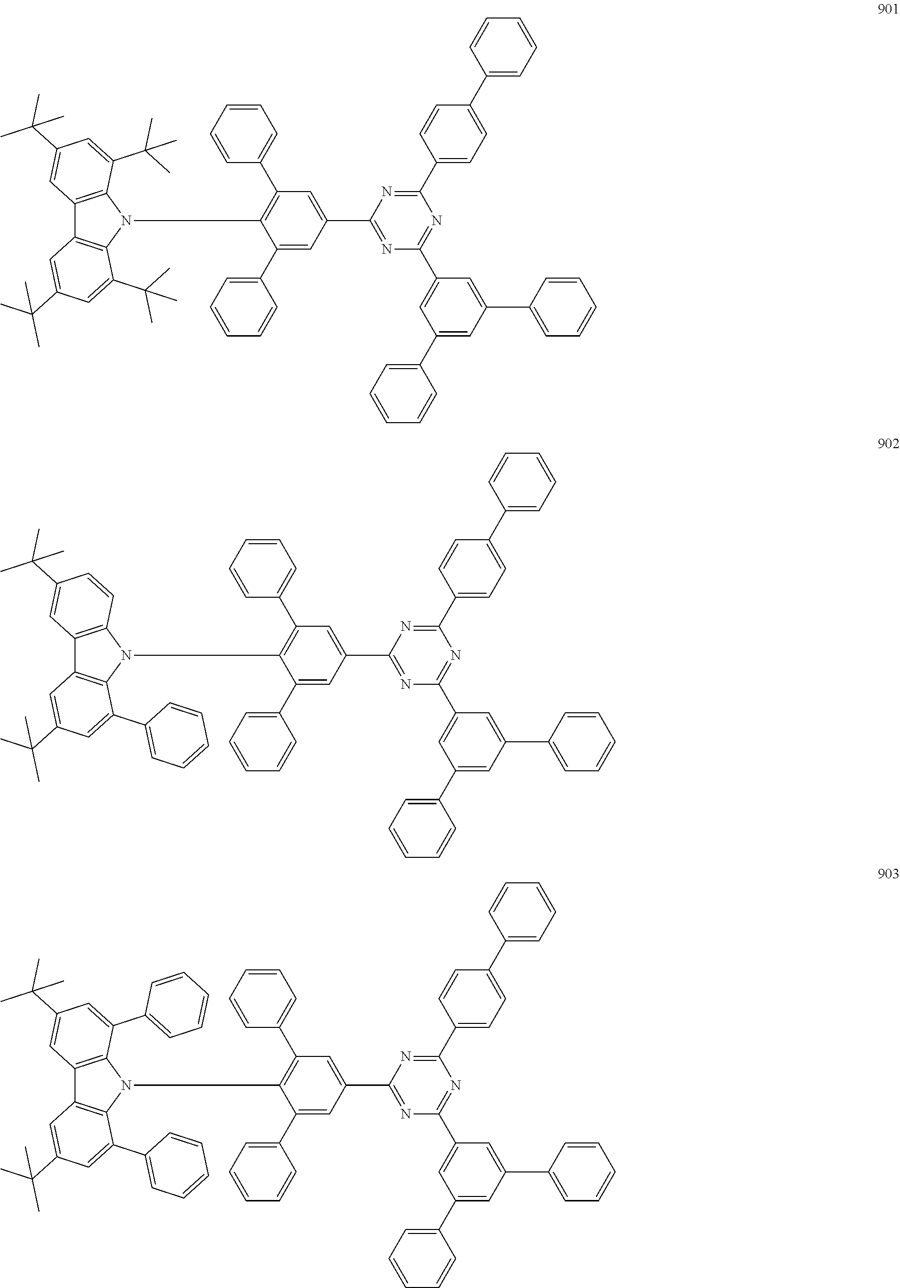 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

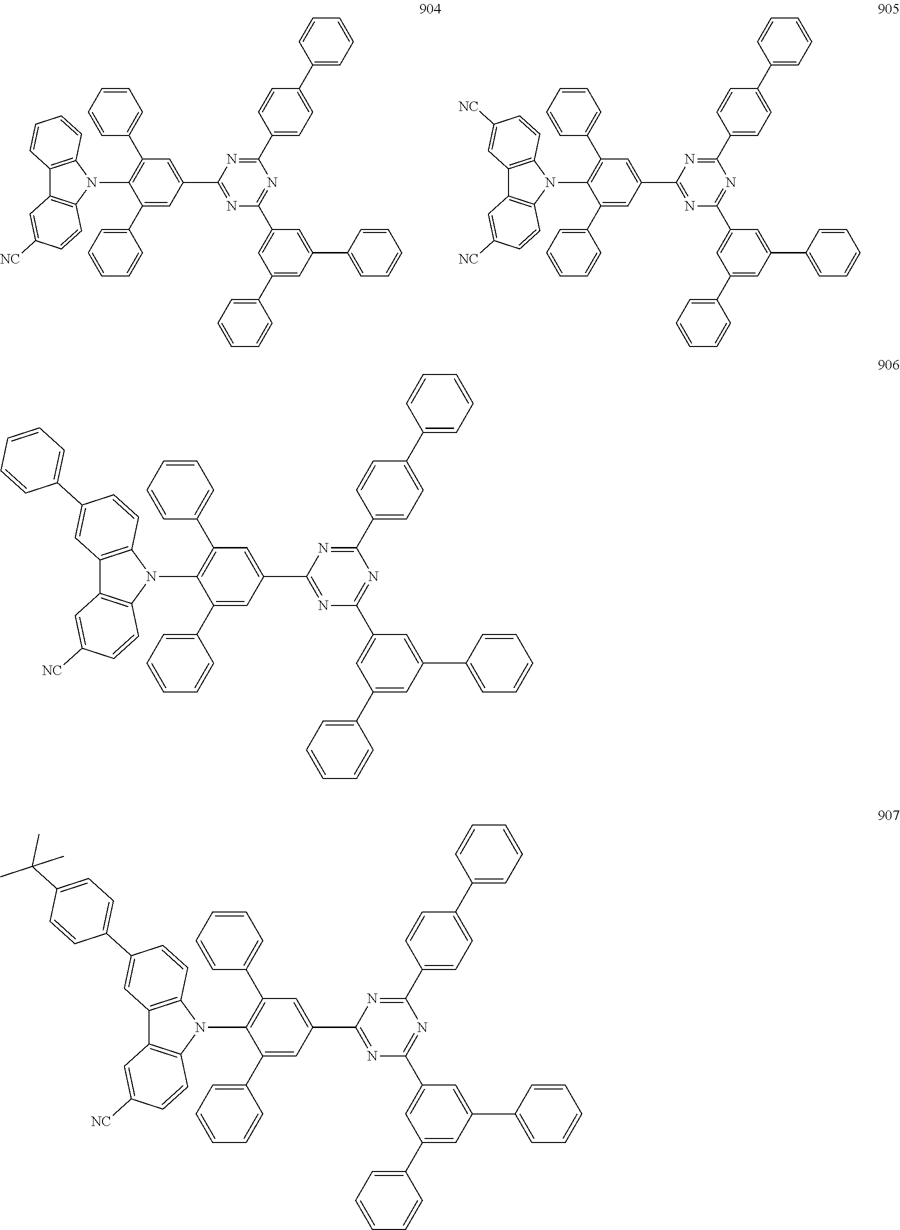

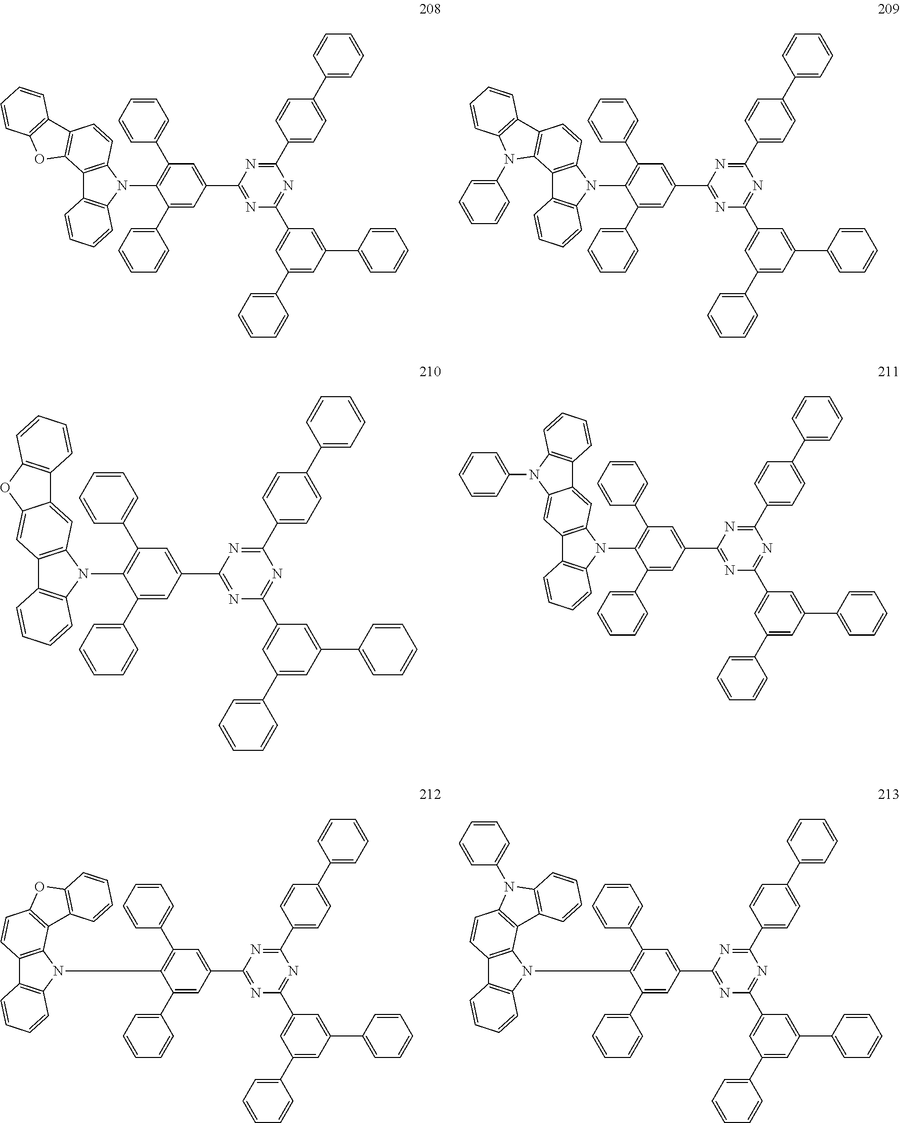

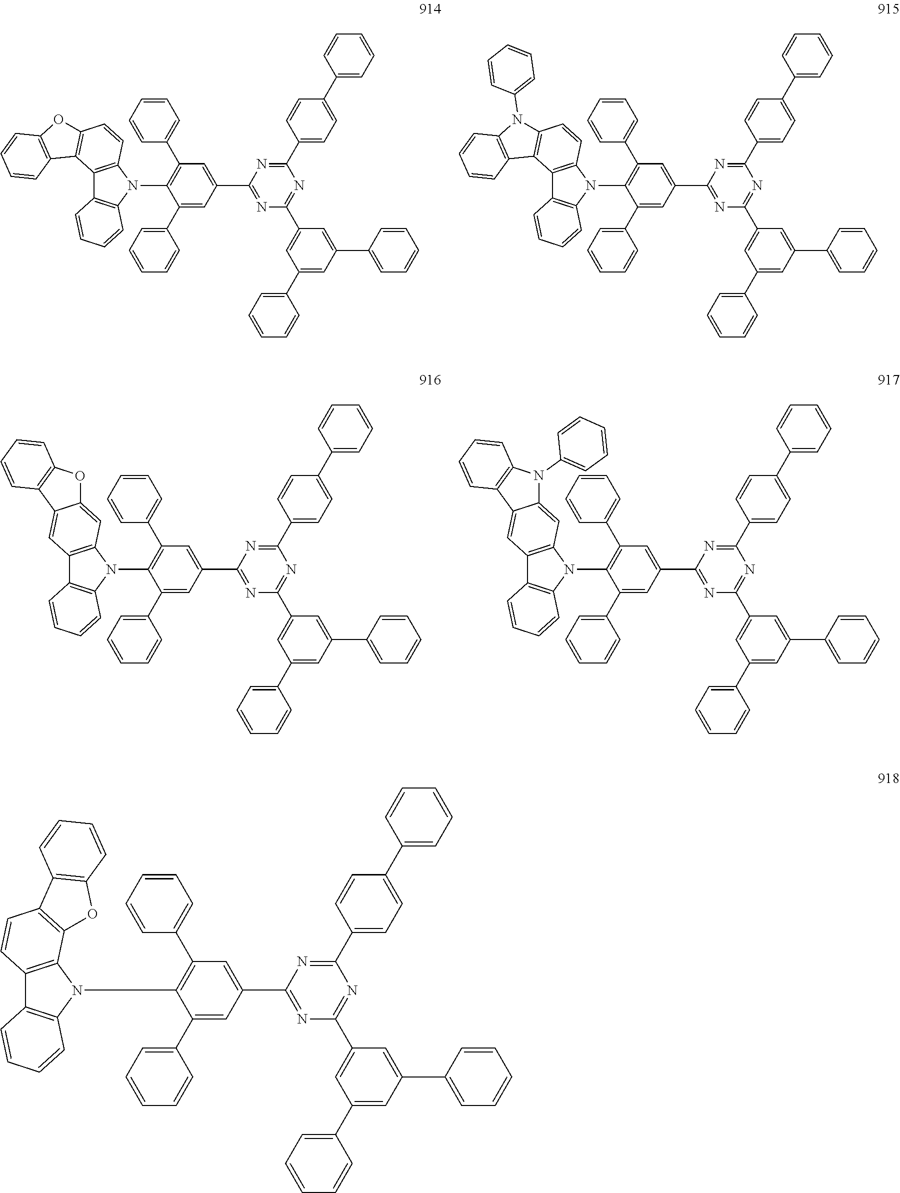

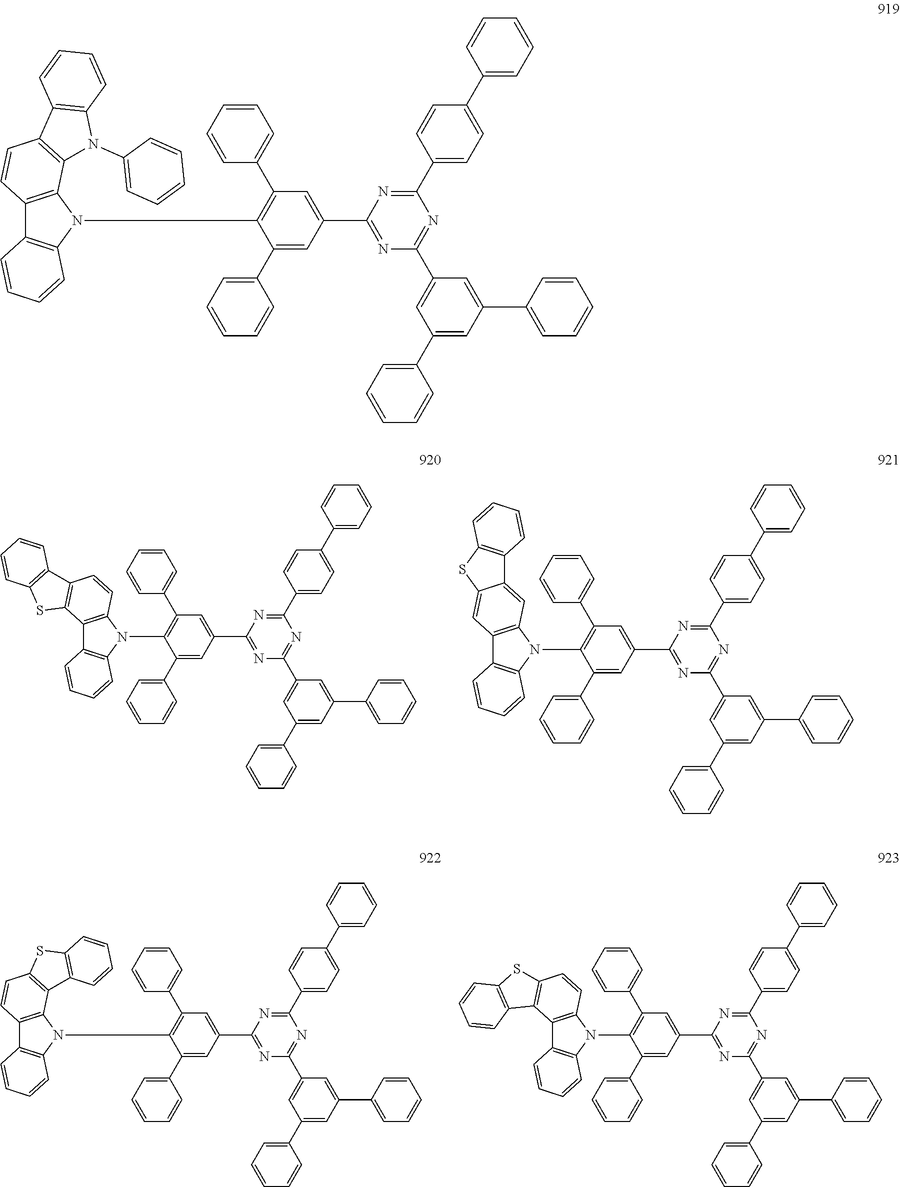

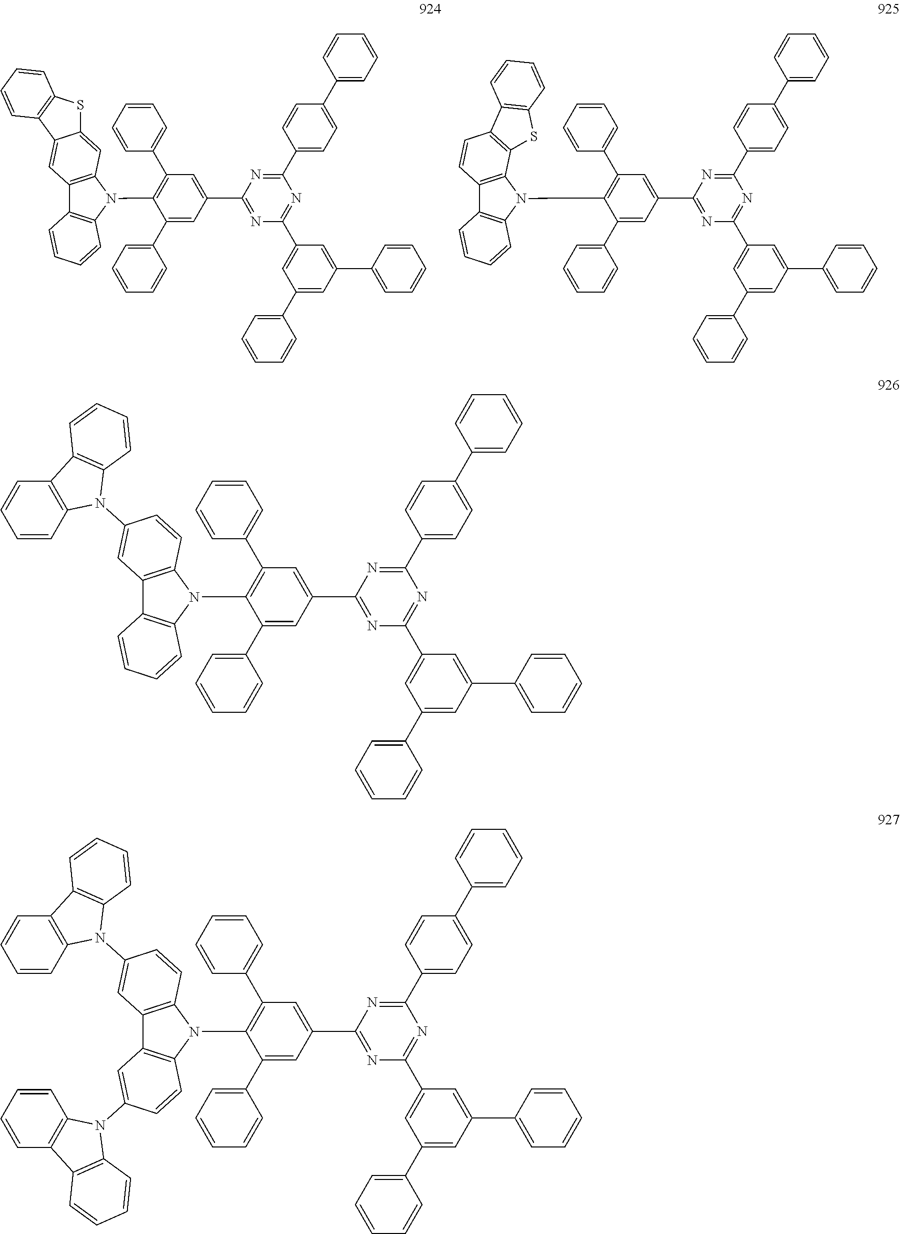

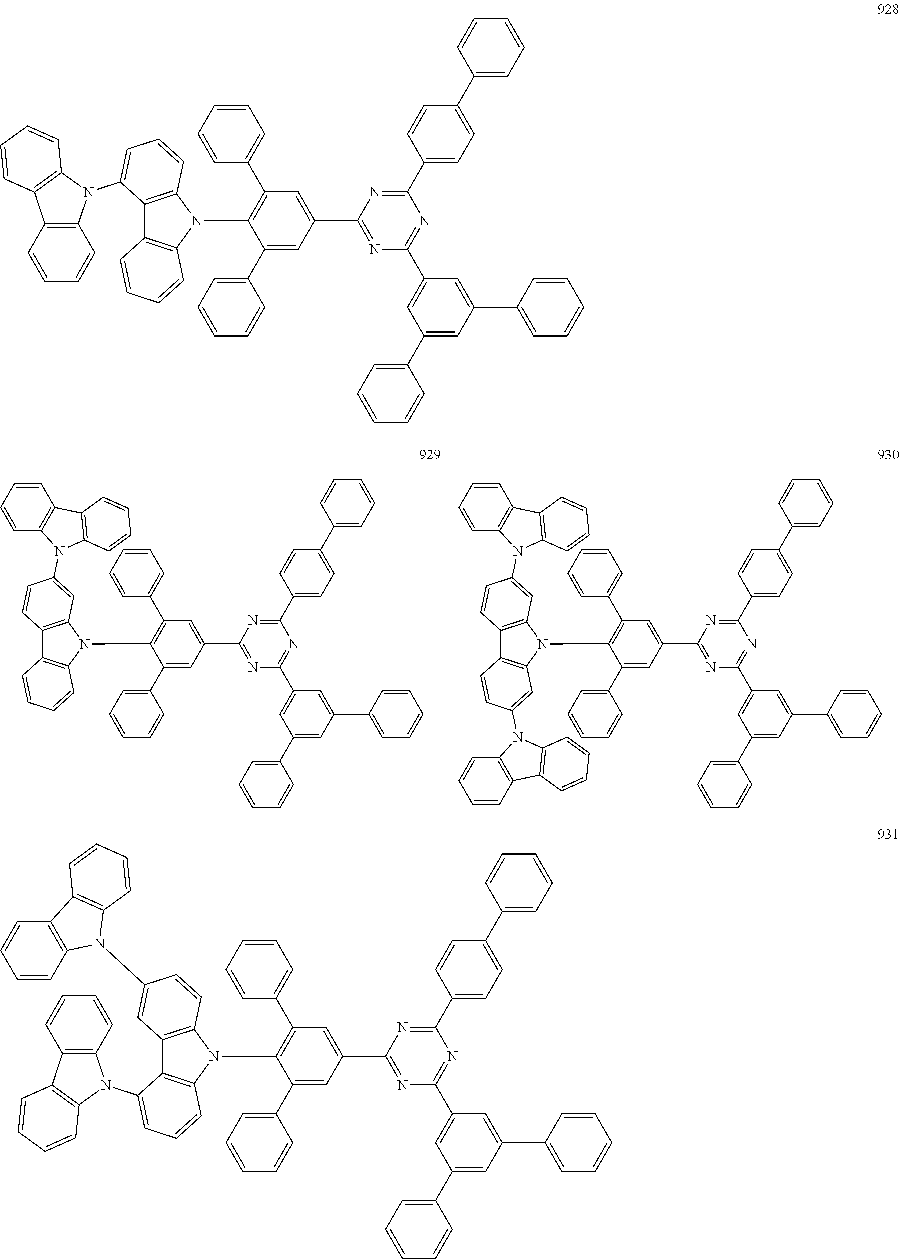

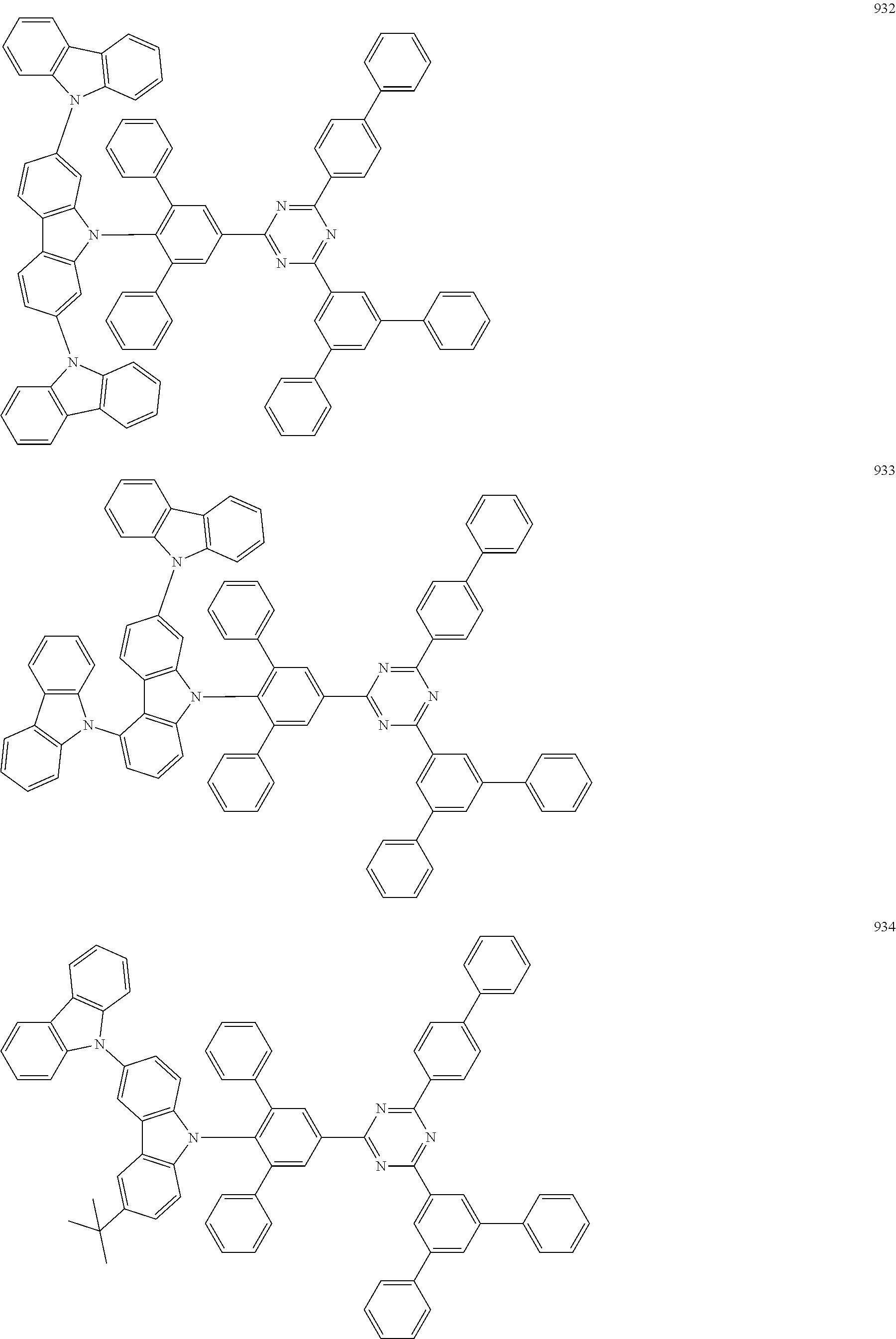 [SMILES: c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6c(-n7c8ccccc8c8ccccc87)cccc65)c(-c5ccccc5)c4)n3)cc2)cc1 ; CC(C)(C)c1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

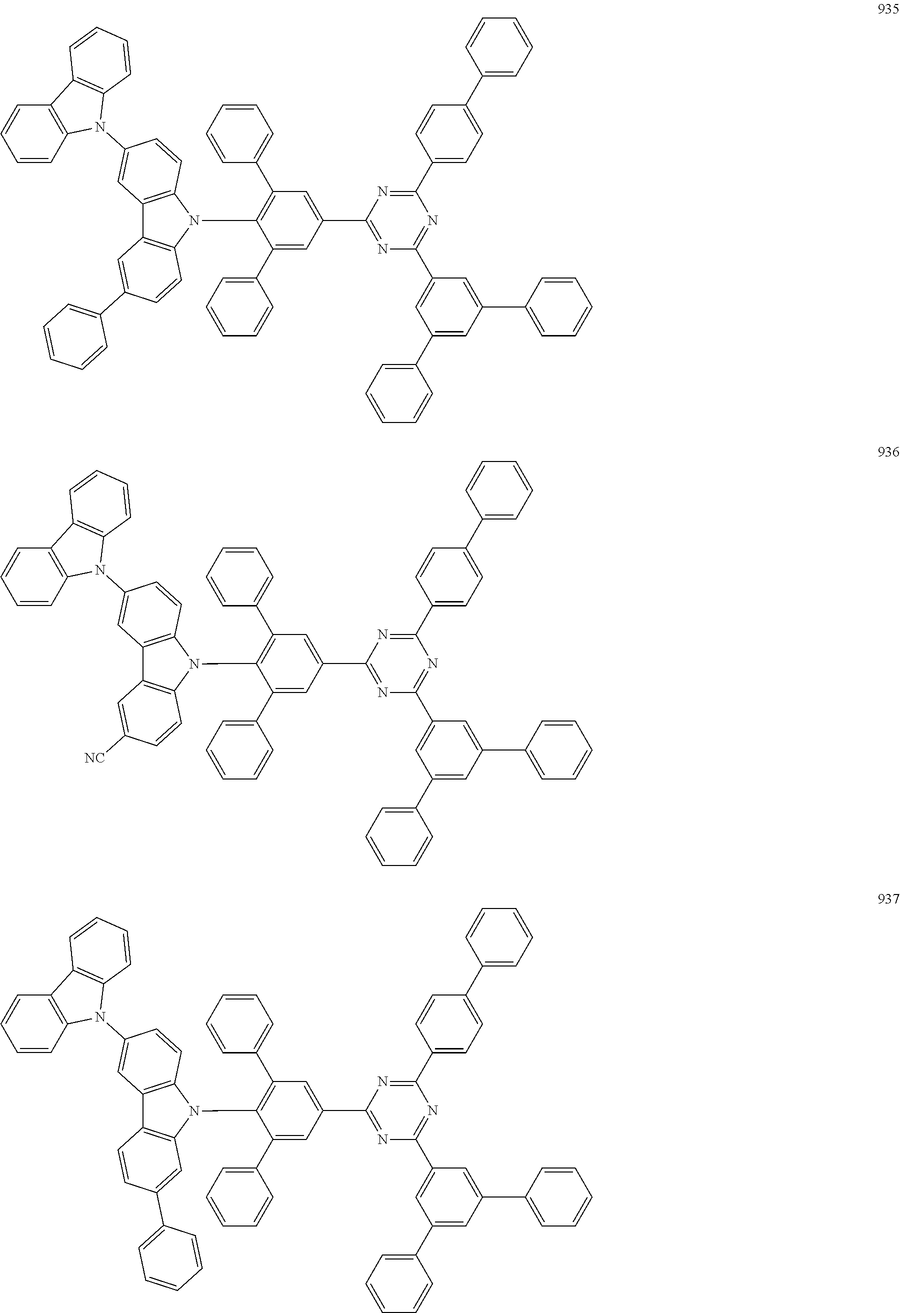 [SMILES: c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-n7c8ccccc8c8ccccc87)ccc65)c(-c5ccccc5)c4)n3)cc2)cc1 ; N#Cc1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-c7ccccc7)cc65)c(-c5ccccc5)c4)n3)cc2)cc1]

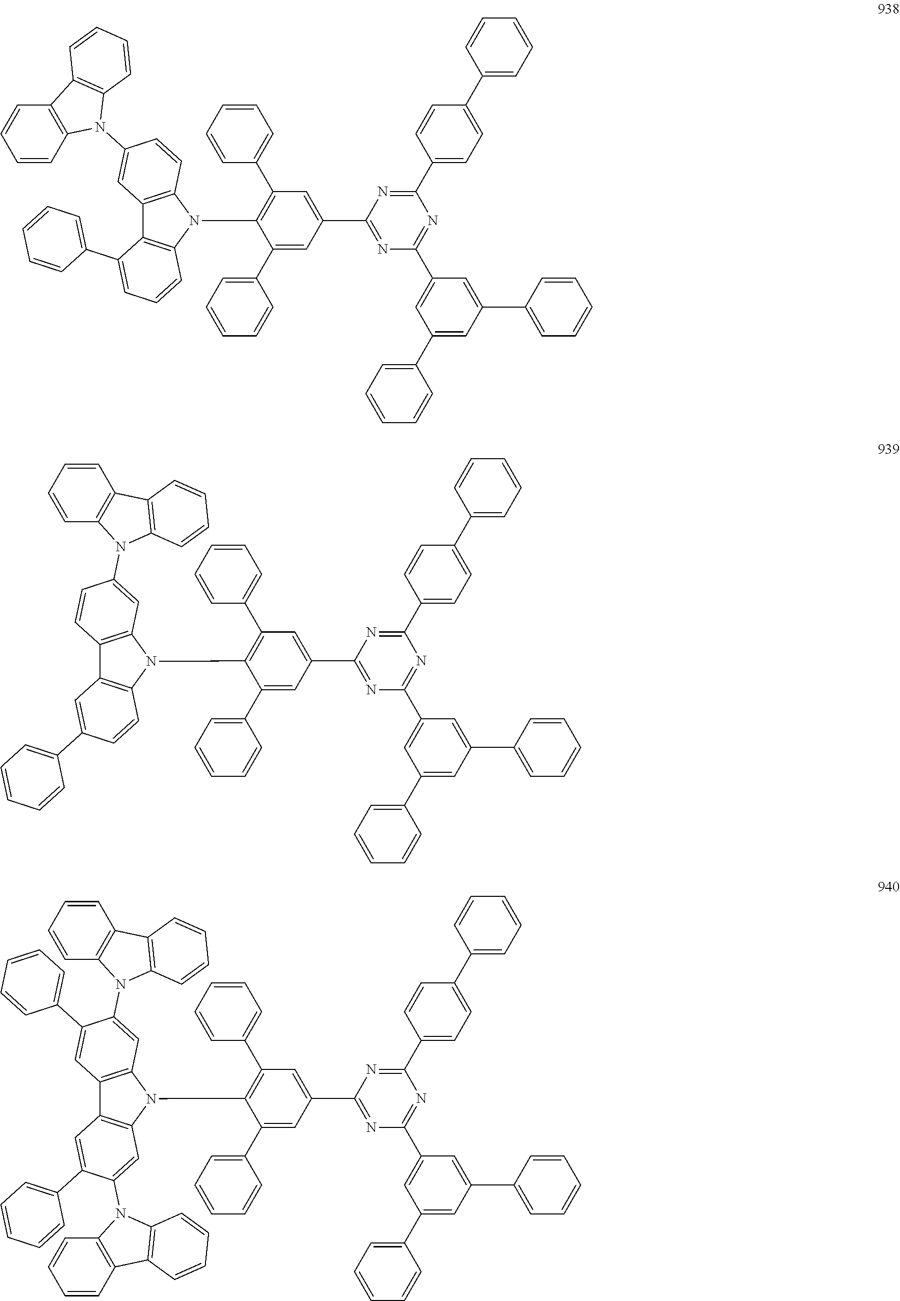 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)c(-c7ccccc7)cc6c6cc(-c7ccccc7)c(-n7c8ccccc8c8ccccc87)cc65)c(-c5ccccc5)c4)n3)cc2)cc1]

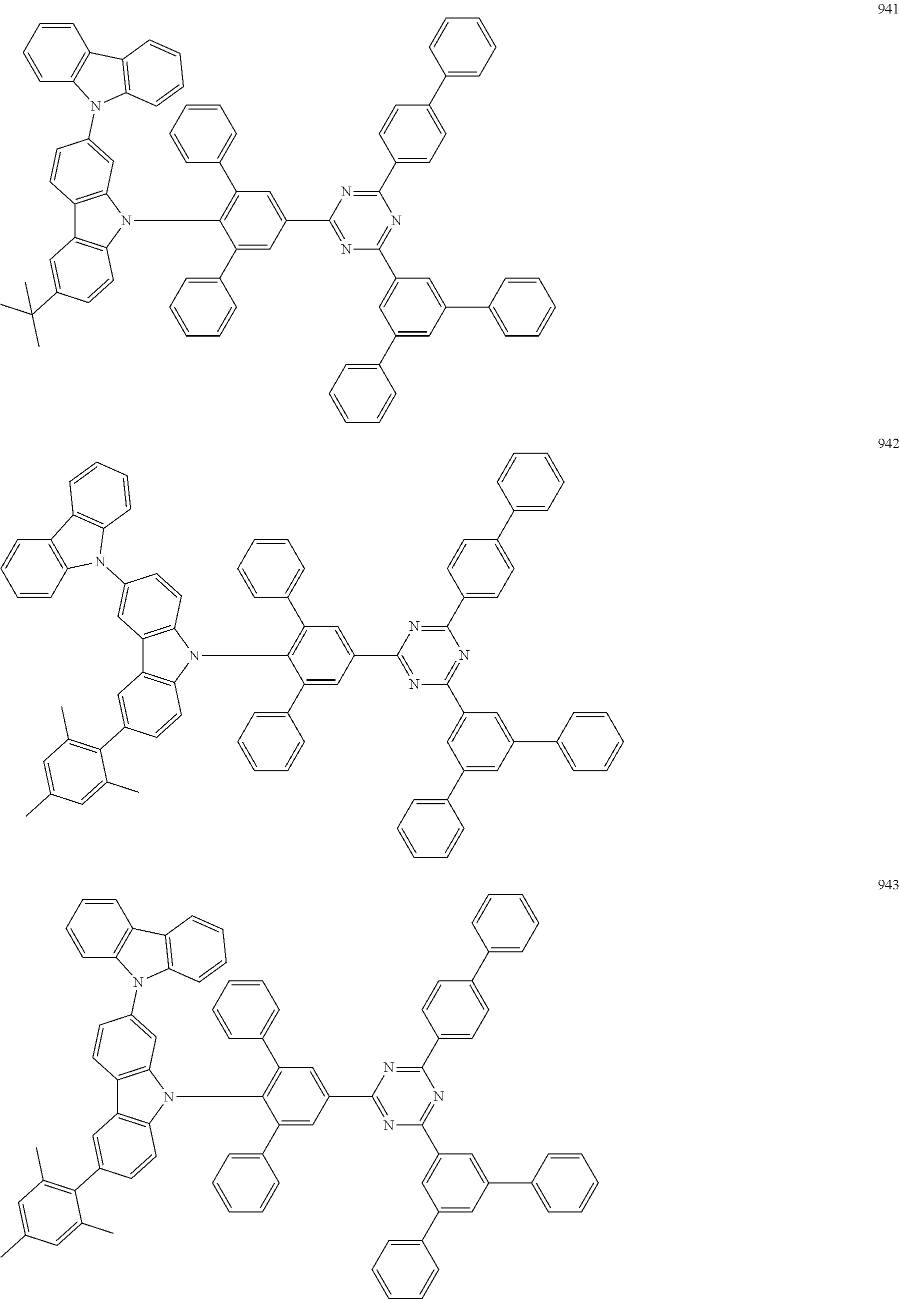 [SMILES: CC(C)(C)c1ccc2c(c1)c1ccc(-n3c4ccccc4c4ccccc43)cc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1]

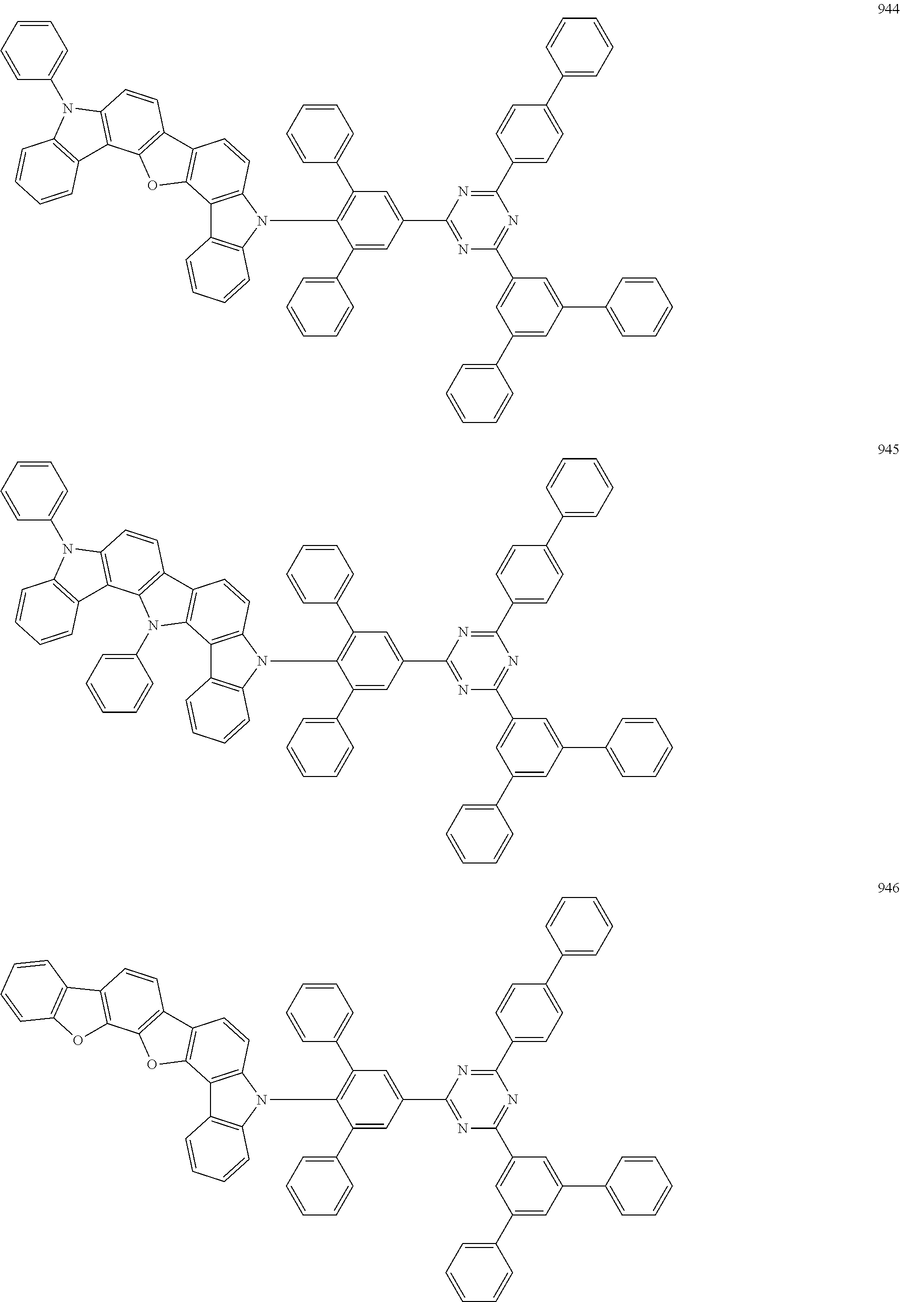 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10oc98)c7ccc65)c(-c5ccccc5)c4)n3)cc2)cc1]

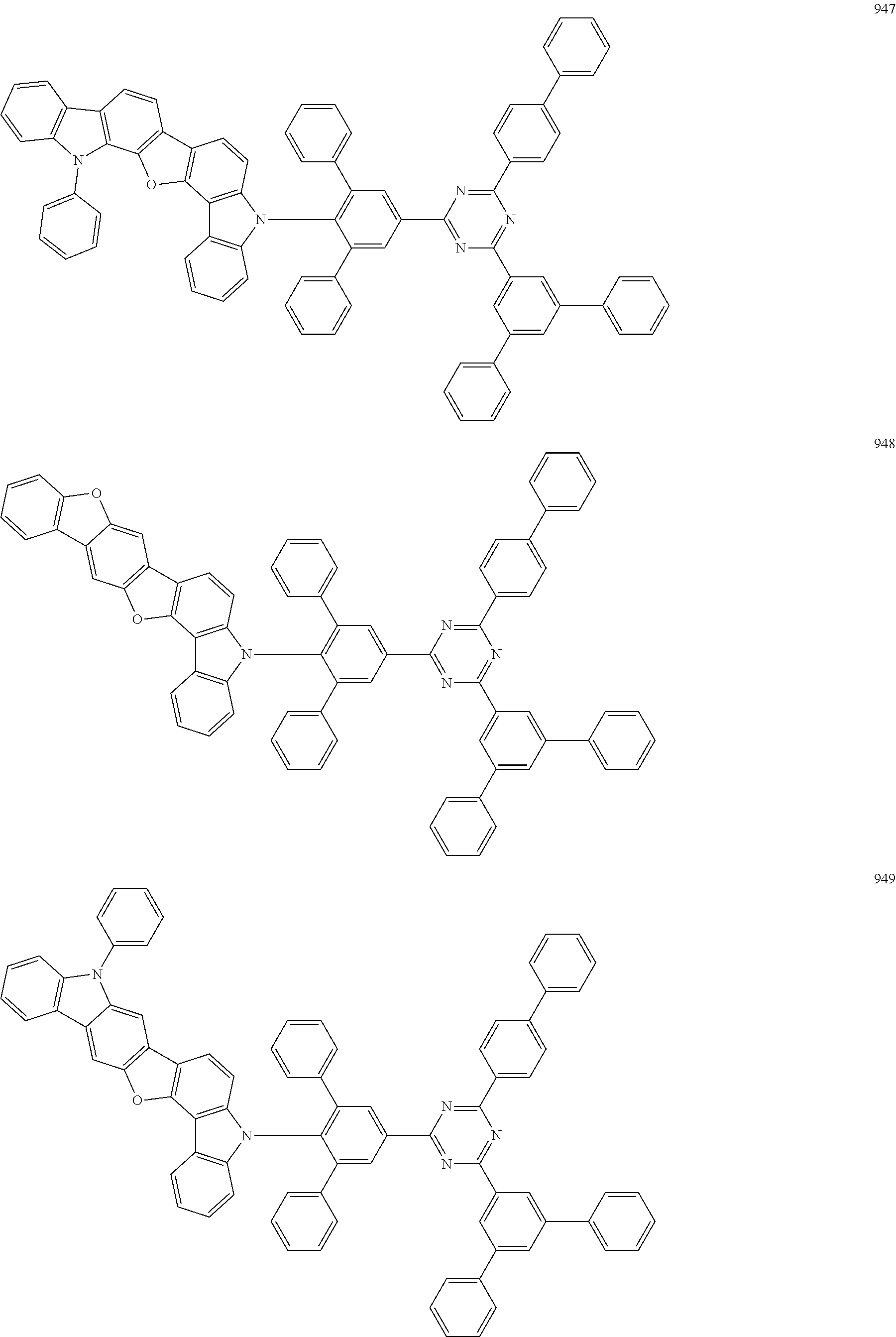 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10n(-c%10ccccc%10)c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)oc5ccccc59)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c%10ccccc%10n(-c%10ccccc%10)c9cc8c7ccc65)c(-c5ccccc5)c4)n3)cc2)cc1]

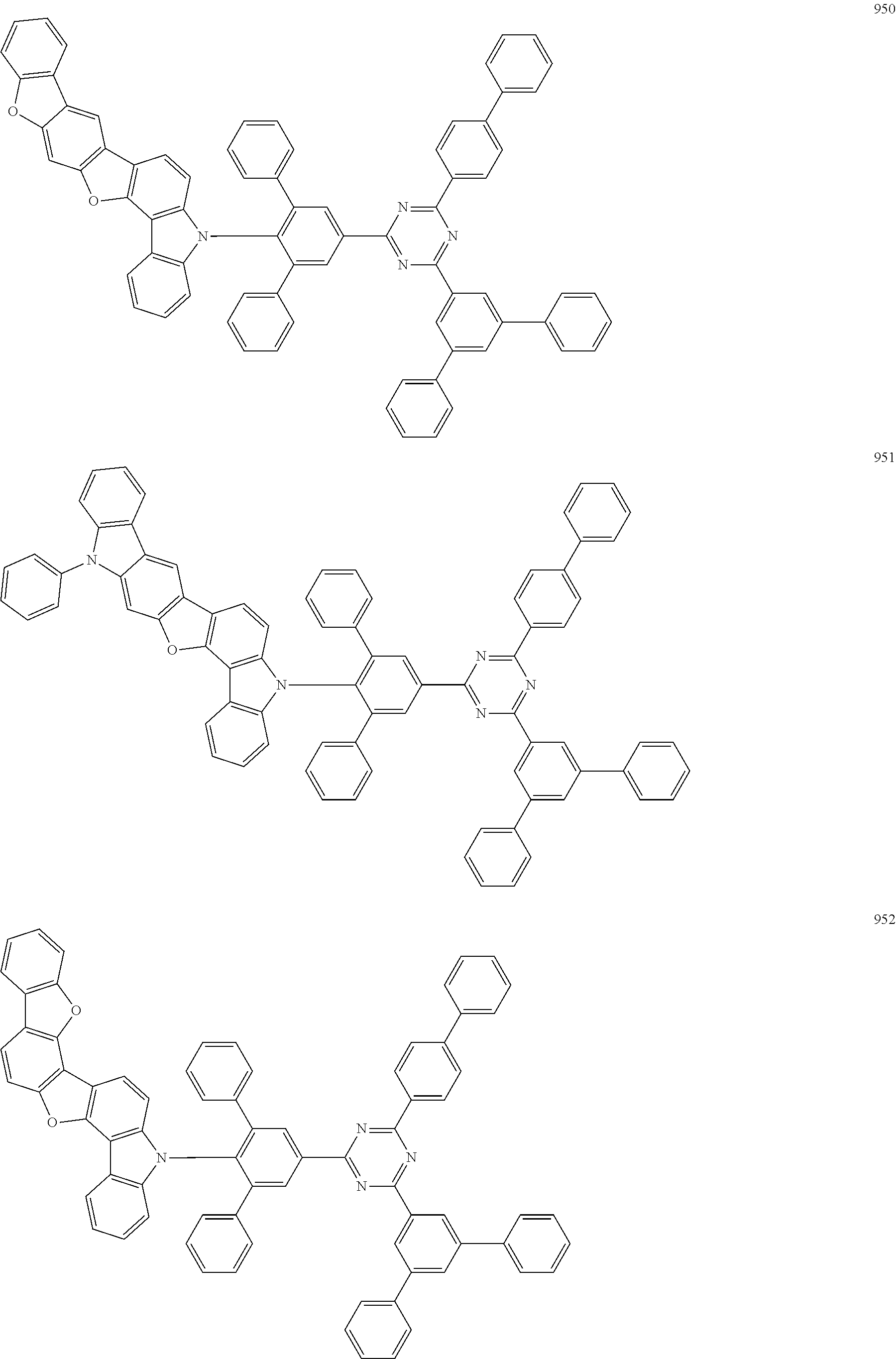 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8cc9oc%10ccccc%10c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)c5ccccc5n9-c5ccccc5)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10oc9c8c7ccc65)c(-c5ccccc5)c4)n3)cc2)cc1]

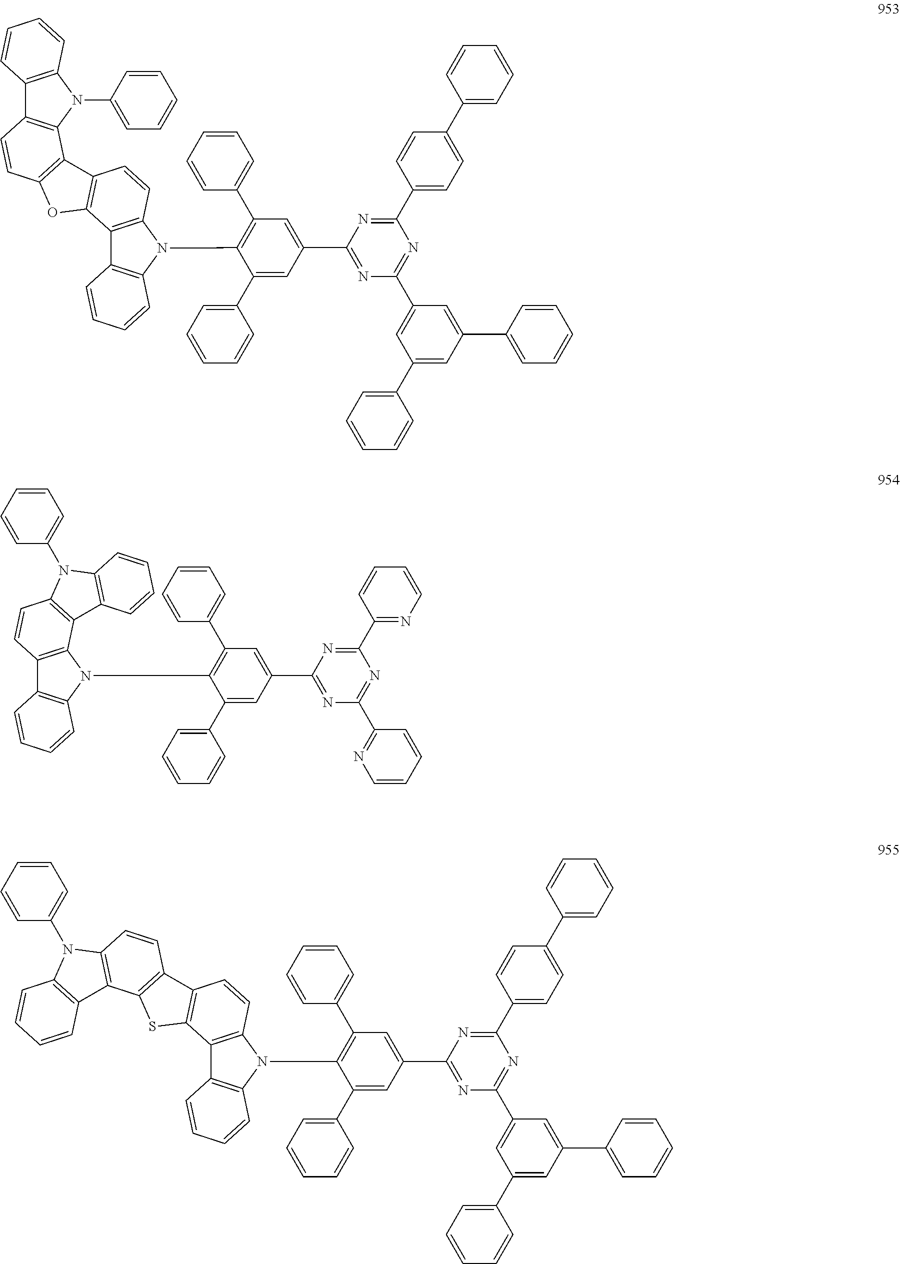 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10n(-c%10ccccc%10)c9c8c7ccc65)c(-c5ccccc5)c4)n3)cc2)cc1 ; c1ccc(-c2cc(-c3nc(-c4ccccn4)nc(-c4ccccn4)n3)cc(-c3ccccc3)c2-n2c3ccccc3c3ccc4c(c5ccccc5n4-c4ccccc4)c32)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1]

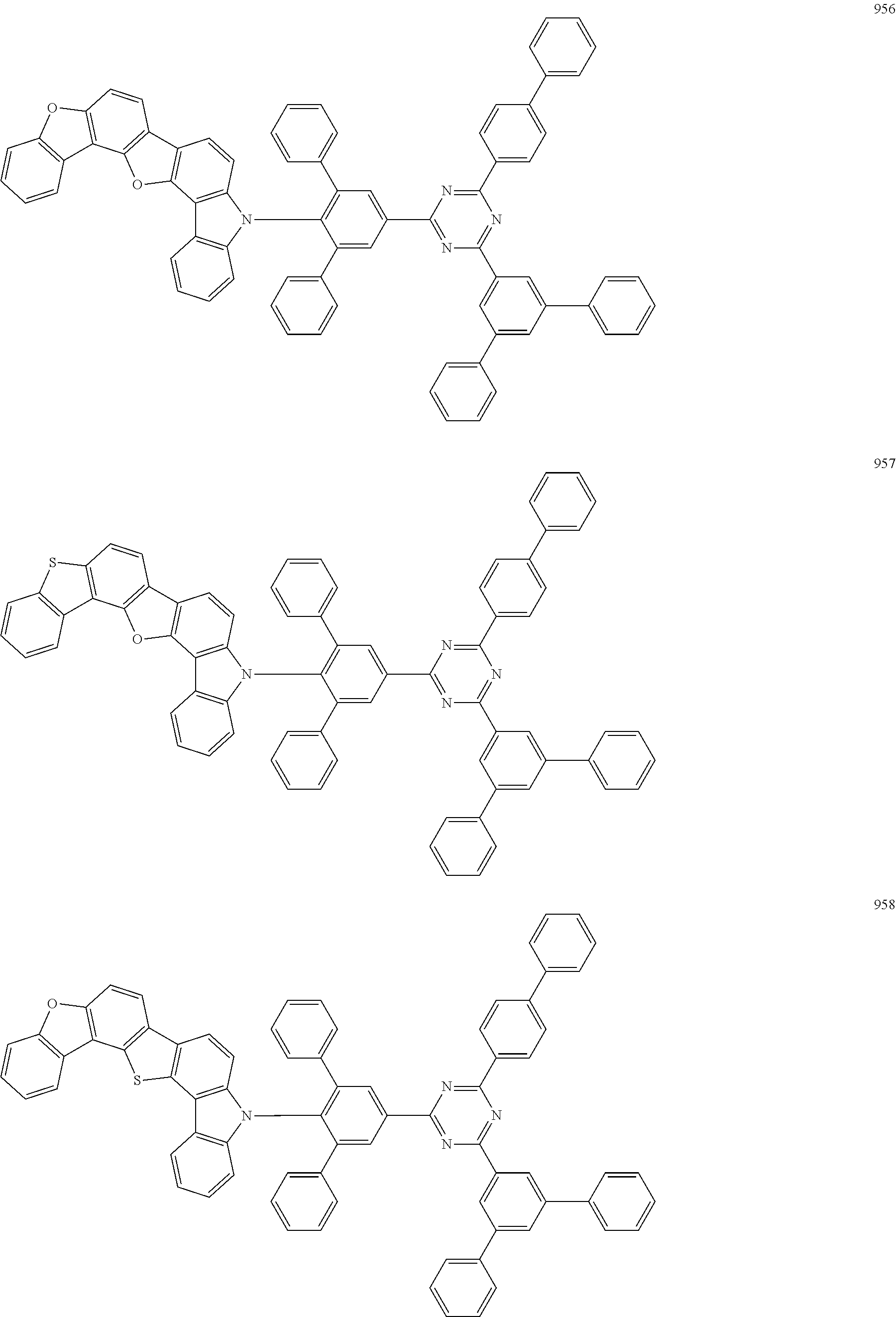 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9oc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9sc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9oc%10ccccc%10c98)c7ccc65)c(-c5ccccc5)c4)n3)cc2)cc1]

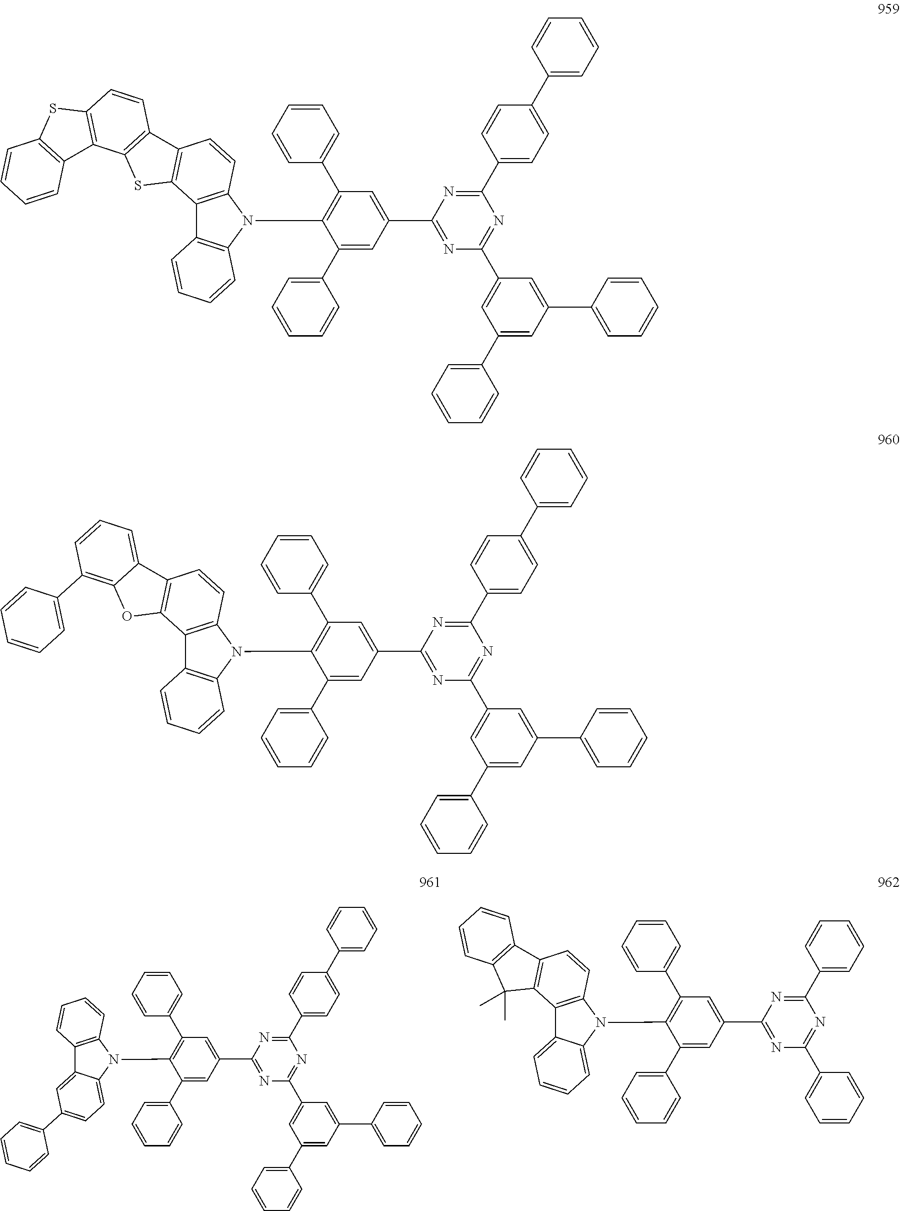

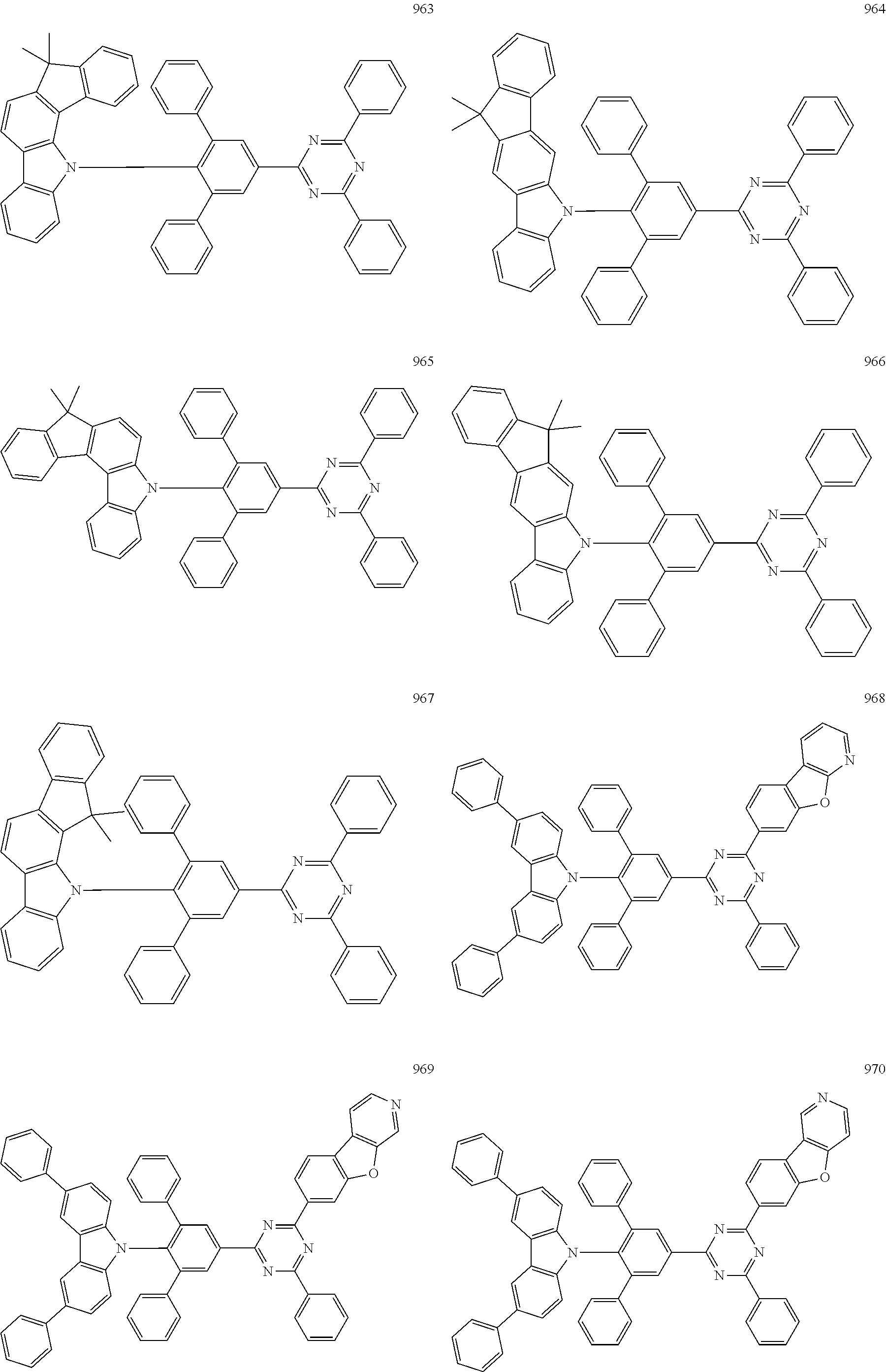

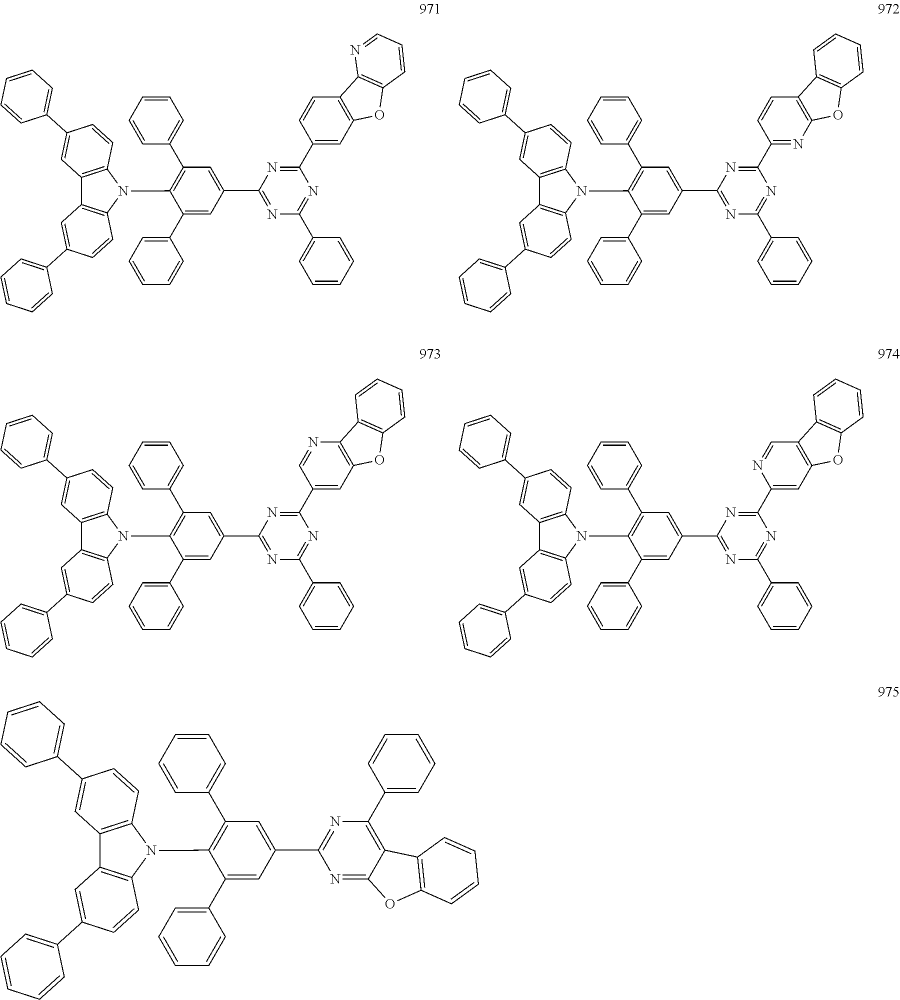

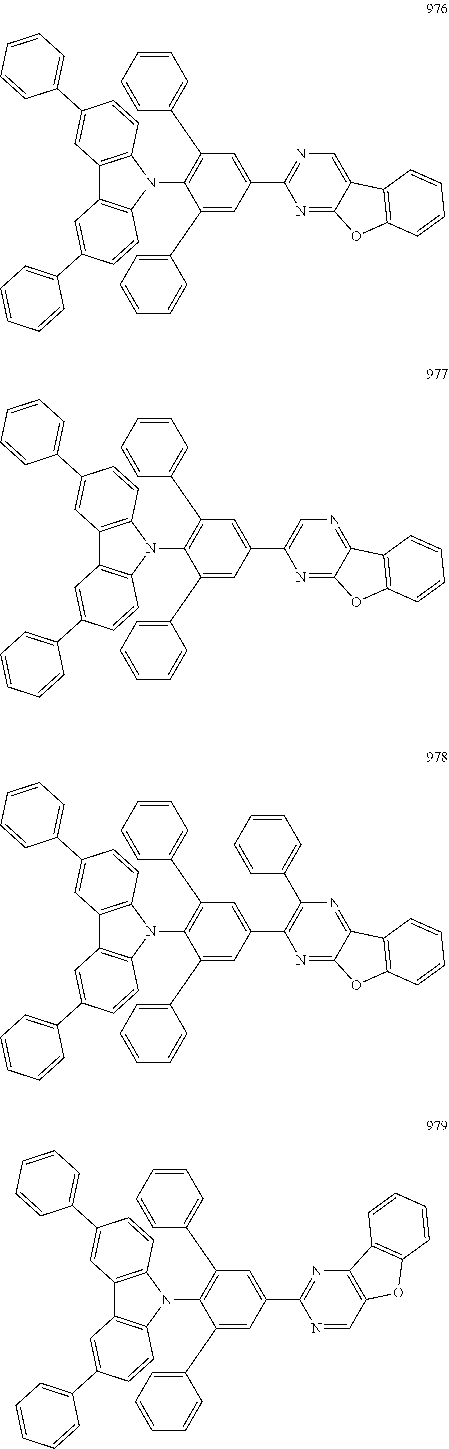 [SMILES: c1ccc(-c2ccc3c(c2)c2cc(-c4ccccc4)ccc2n3-c2c(-c3ccccc3)cc(-c3ncc4c(n3)oc3ccccc34)cc2-c2ccccc2)cc1 ; c1ccc(-c2ccc3c(c2)c2cc(-c4ccccc4)ccc2n3-c2c(-c3ccccc3)cc(-c3cnc4c(n3)oc3ccccc34)cc2-c2ccccc2)cc1 ; c1ccc(-c2ccc3c(c2)c2cc(-c4ccccc4)ccc2n3-c2c(-c3ccccc3)cc(-c3nc4oc5ccccc5c4nc3-c3ccccc3)cc2-c2ccccc2)cc1 ; c1ccc(-c2ccc3c(c2)c2cc(-c4ccccc4)ccc2n3-c2c(-c3ccccc3)cc(-c3ncc4oc5ccccc5c4n3)cc2-c2ccccc2)cc1]

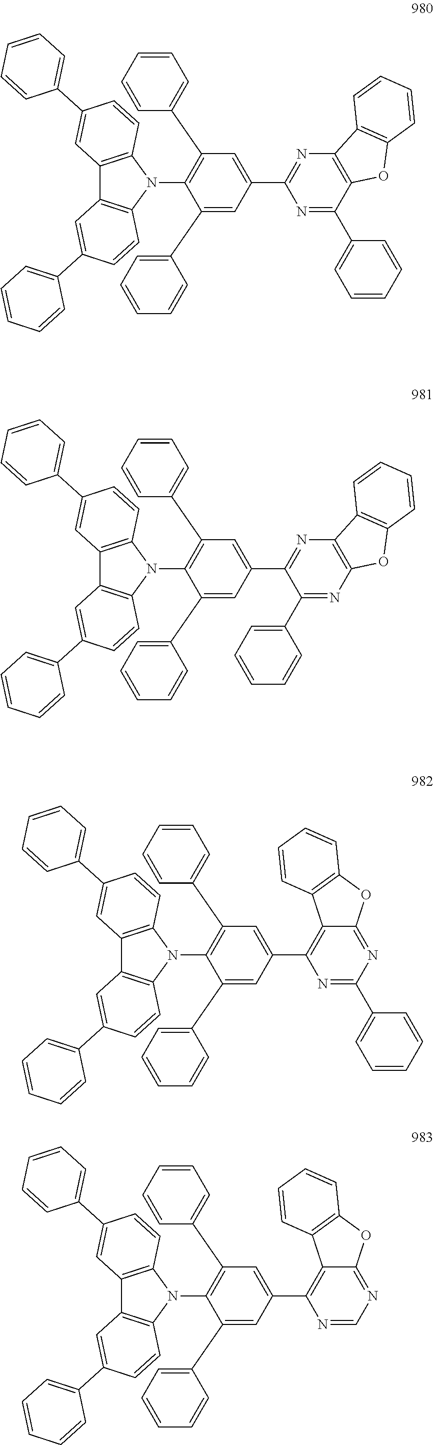

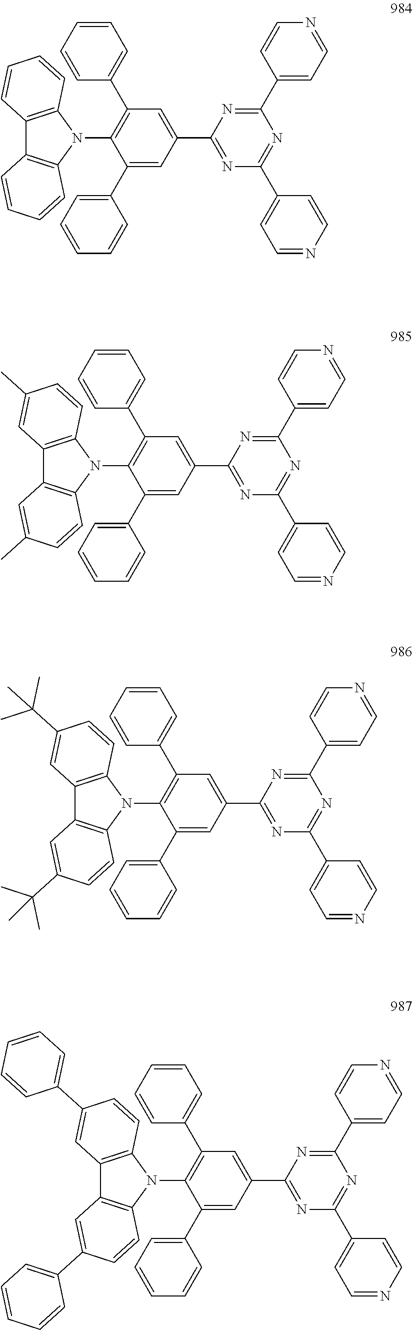 [SMILES: c1ccc(-c2cc(-c3nc(-c4ccncc4)nc(-c4ccncc4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccncc3)nc(-c3ccncc3)n2)cc1-c1ccccc1 ; CC(C)(C)c1ccc2c(c1)c1cc(C(C)(C)C)ccc1n2-c1c(-c2ccccc2)cc(-c2nc(-c3ccncc3)nc(-c3ccncc3)n2)cc1-c1ccccc1 ; c1ccc(-c2ccc3c(c2)c2cc(-c4ccccc4)ccc2n3-c2c(-c3ccccc3)cc(-c3nc(-c4ccncc4)nc(-c4ccncc4)n3)cc2-c2ccccc2)cc1]

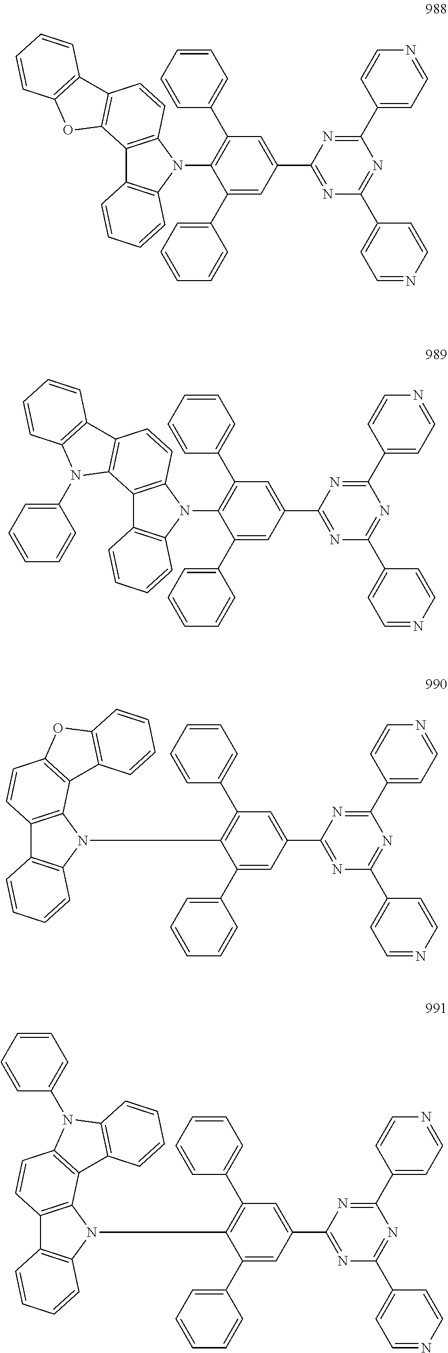 [SMILES: c1ccc(-c2cc(-c3nc(-c4ccncc4)nc(-c4ccncc4)n3)cc(-c3ccccc3)c2-n2c3ccccc3c3c4oc5ccccc5c4ccc32)cc1 ; c1ccc(-c2cc(-c3nc(-c4ccncc4)nc(-c4ccncc4)n3)cc(-c3ccccc3)c2-n2c3ccccc3c3c2ccc2c4ccccc4n(-c4ccccc4)c23)cc1 ; c1ccc(-c2cc(-c3nc(-c4ccncc4)nc(-c4ccncc4)n3)cc(-c3ccccc3)c2-n2c3ccccc3c3ccc4oc5ccccc5c4c32)cc1 ; c1ccc(-c2cc(-c3nc(-c4ccncc4)nc(-c4ccncc4)n3)cc(-c3ccccc3)c2-n2c3ccccc3c3ccc4c(c5ccccc5n4-c4ccccc4)c32)cc1]

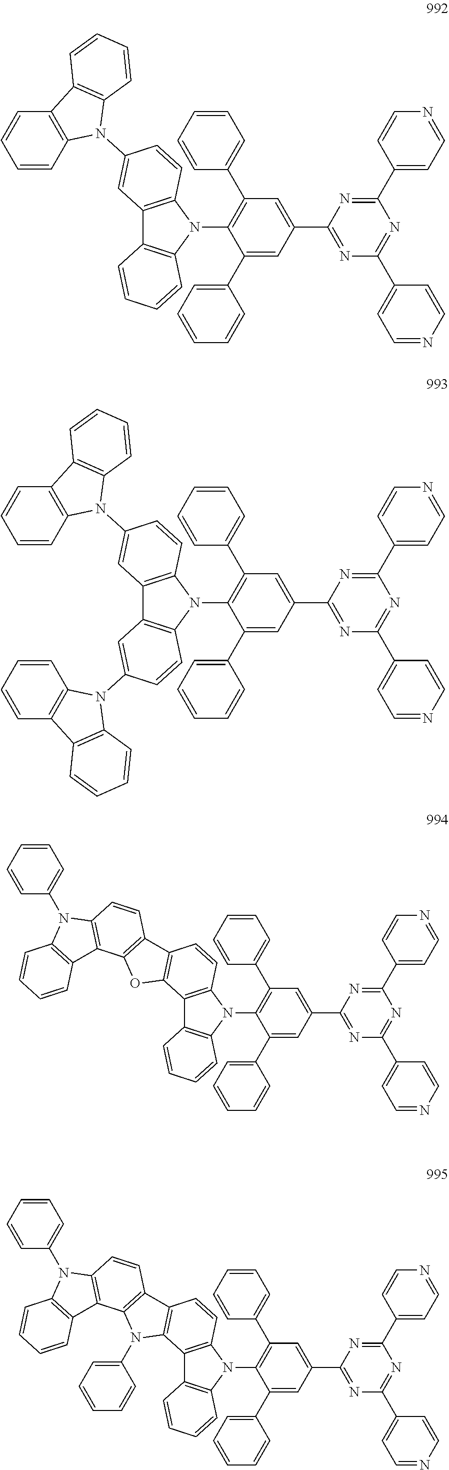 [SMILES: c1ccc(-c2cc(-c3nc(-c4ccncc4)nc(-c4ccncc4)n3)cc(-c3ccccc3)c2-n2c3ccccc3c3cc(-n4c5ccccc5c5ccccc54)ccc32)cc1 ; c1ccc(-c2cc(-c3nc(-c4ccncc4)nc(-c4ccncc4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2cc(-c3nc(-c4ccncc4)nc(-c4ccncc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4ccncc4)nc(-c4ccncc4)n3)cc(-c3ccccc3)c2-n2c3ccccc3c3c2ccc2c4ccc5c(c6ccccc6n5-c5ccccc5)c4n(-c4ccccc4)c23)cc1]

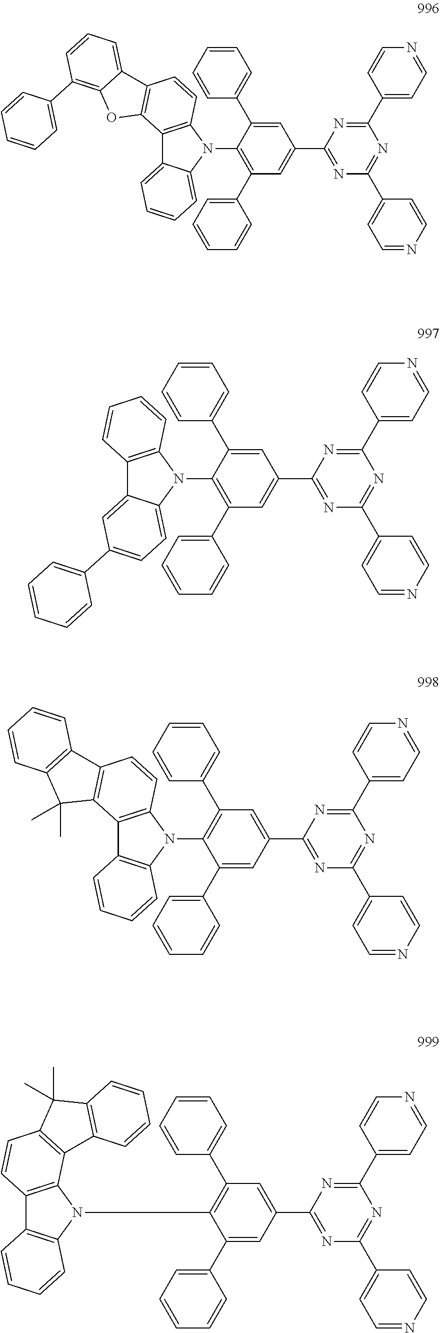 [SMILES: c1ccc(-c2cc(-c3nc(-c4ccncc4)nc(-c4ccncc4)n3)cc(-c3ccccc3)c2-n2c3ccccc3c3c4oc5c(-c6ccccc6)cccc5c4ccc32)cc1 ; c1ccc(-c2ccc3c(c2)c2ccccc2n3-c2c(-c3ccccc3)cc(-c3nc(-c4ccncc4)nc(-c4ccncc4)n3)cc2-c2ccccc2)cc1 ; CC1(C)c2ccccc2-c2ccc3c(c21)c1ccccc1n3-c1c(-c2ccccc2)cc(-c2nc(-c3ccncc3)nc(-c3ccncc3)n2)cc1-c1ccccc1 ; CC1(C)c2ccccc2-c2c1ccc1c3ccccc3n(-c3c(-c4ccccc4)cc(-c4nc(-c5ccncc5)nc(-c5ccncc5)n4)cc3-c3ccccc3)c21]

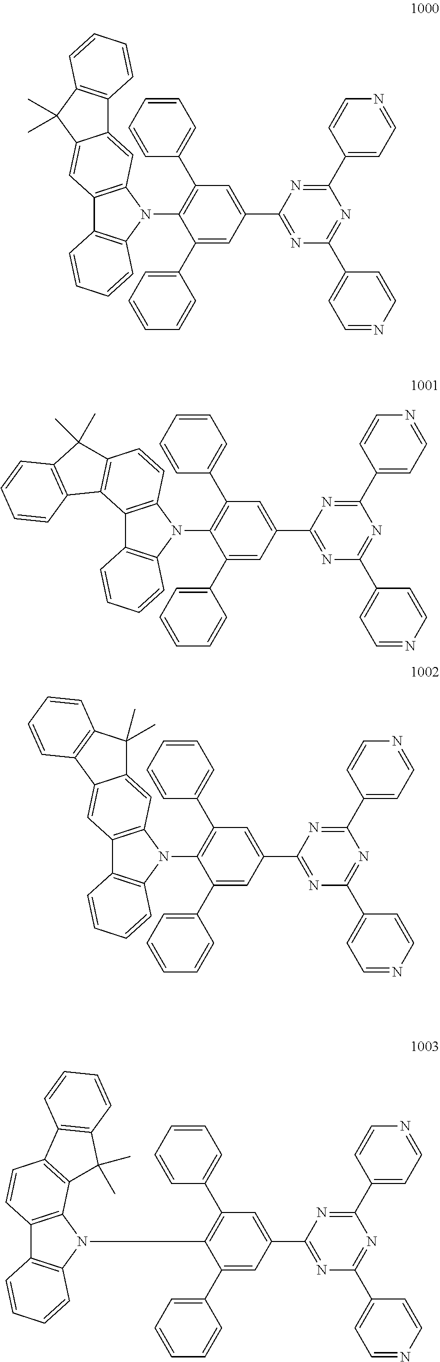 [SMILES: CC1(C)c2ccccc2-c2cc3c(cc21)c1ccccc1n3-c1c(-c2ccccc2)cc(-c2nc(-c3ccncc3)nc(-c3ccncc3)n2)cc1-c1ccccc1 ; CC1(C)c2ccccc2-c2c1ccc1c2c2ccccc2n1-c1c(-c2ccccc2)cc(-c2nc(-c3ccncc3)nc(-c3ccncc3)n2)cc1-c1ccccc1 ; CC1(C)c2ccccc2-c2cc3c4ccccc4n(-c4c(-c5ccccc5)cc(-c5nc(-c6ccncc6)nc(-c6ccncc6)n5)cc4-c4ccccc4)c3cc21 ; CC1(C)c2ccccc2-c2ccc3c4ccccc4n(-c4c(-c5ccccc5)cc(-c5nc(-c6ccncc6)nc(-c6ccncc6)n5)cc4-c4ccccc4)c3c21]

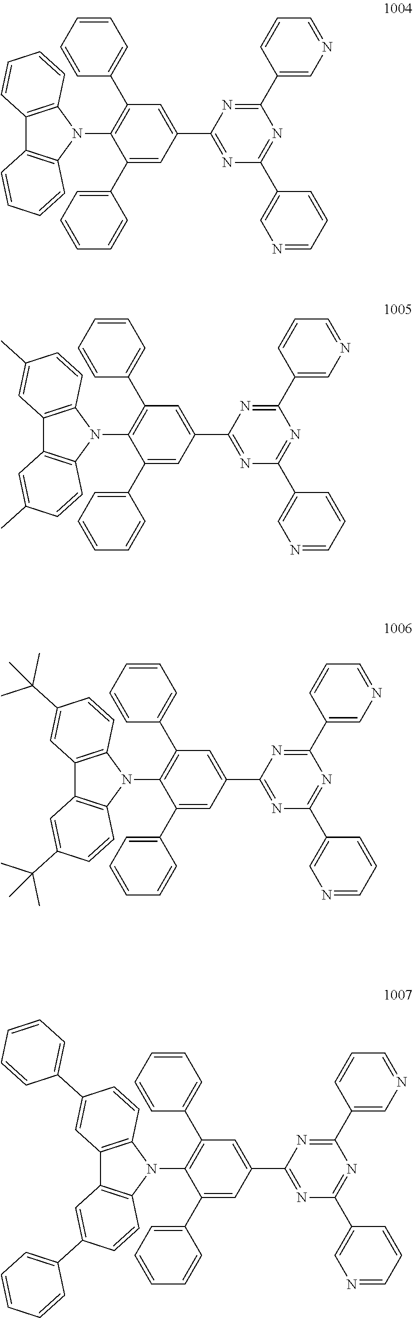 [SMILES: c1ccc(-c2cc(-c3nc(-c4cccnc4)nc(-c4cccnc4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3cccnc3)nc(-c3cccnc3)n2)cc1-c1ccccc1 ; CC(C)(C)c1ccc2c(c1)c1cc(C(C)(C)C)ccc1n2-c1c(-c2ccccc2)cc(-c2nc(-c3cccnc3)nc(-c3cccnc3)n2)cc1-c1ccccc1 ; c1ccc(-c2ccc3c(c2)c2cc(-c4ccccc4)ccc2n3-c2c(-c3ccccc3)cc(-c3nc(-c4cccnc4)nc(-c4cccnc4)n3)cc2-c2ccccc2)cc1]

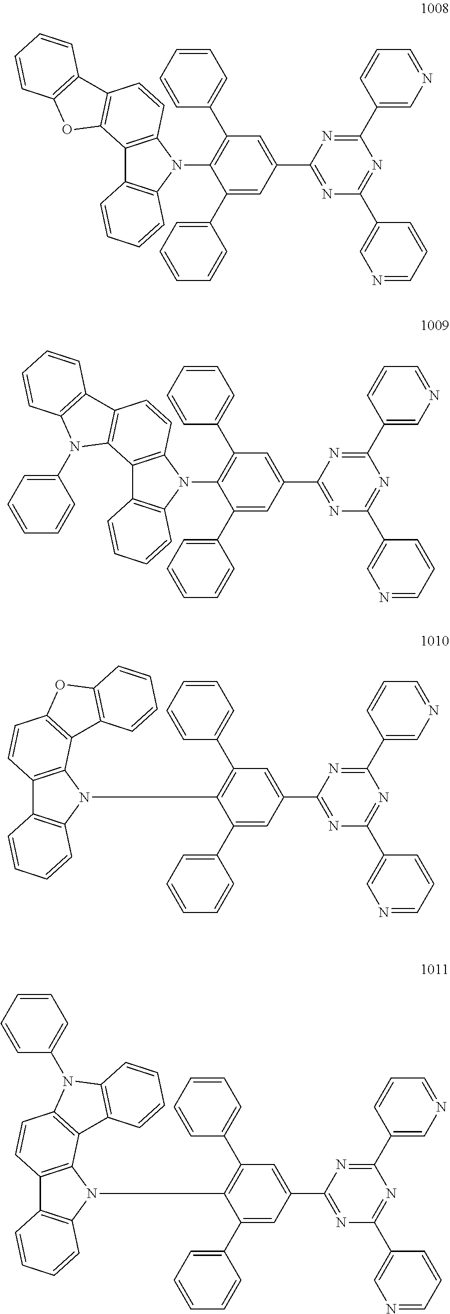 [SMILES: c1ccc(-c2cc(-c3nc(-c4cccnc4)nc(-c4cccnc4)n3)cc(-c3ccccc3)c2-n2c3ccccc3c3c4oc5ccccc5c4ccc32)cc1 ; c1ccc(-c2cc(-c3nc(-c4cccnc4)nc(-c4cccnc4)n3)cc(-c3ccccc3)c2-n2c3ccccc3c3c2ccc2c4ccccc4n(-c4ccccc4)c23)cc1 ; c1ccc(-c2cc(-c3nc(-c4cccnc4)nc(-c4cccnc4)n3)cc(-c3ccccc3)c2-n2c3ccccc3c3ccc4oc5ccccc5c4c32)cc1 ; c1ccc(-c2cc(-c3nc(-c4cccnc4)nc(-c4cccnc4)n3)cc(-c3ccccc3)c2-n2c3ccccc3c3ccc4c(c5ccccc5n4-c4ccccc4)c32)cc1]

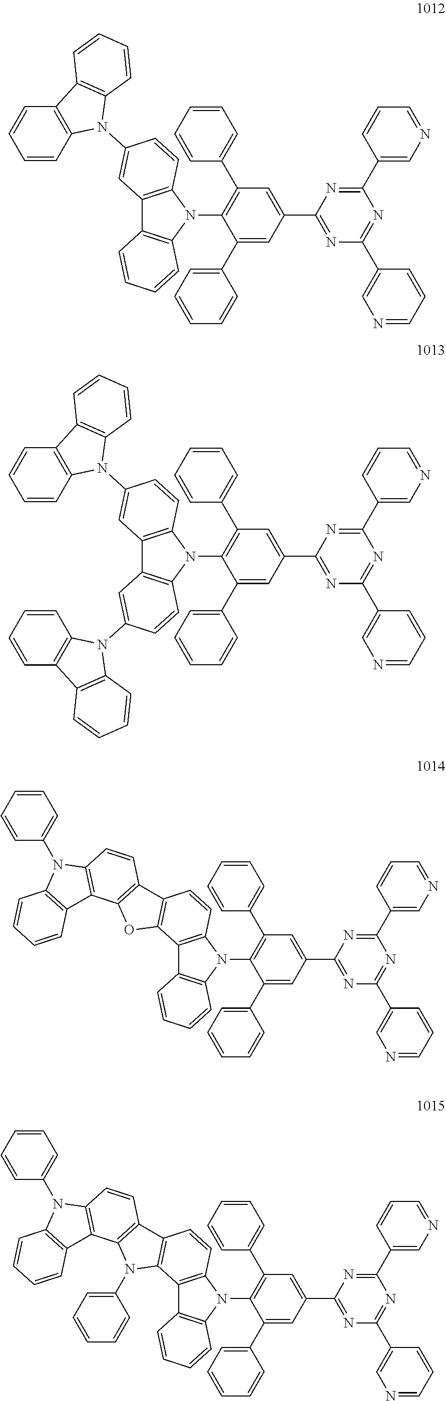 [SMILES: c1ccc(-c2cc(-c3nc(-c4cccnc4)nc(-c4cccnc4)n3)cc(-c3ccccc3)c2-n2c3ccccc3c3cc(-n4c5ccccc5c5ccccc54)ccc32)cc1 ; c1ccc(-c2cc(-c3nc(-c4cccnc4)nc(-c4cccnc4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2cc(-c3nc(-c4cccnc4)nc(-c4cccnc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4cccnc4)nc(-c4cccnc4)n3)cc(-c3ccccc3)c2-n2c3ccccc3c3c2ccc2c4ccc5c(c6ccccc6n5-c5ccccc5)c4n(-c4ccccc4)c23)cc1]

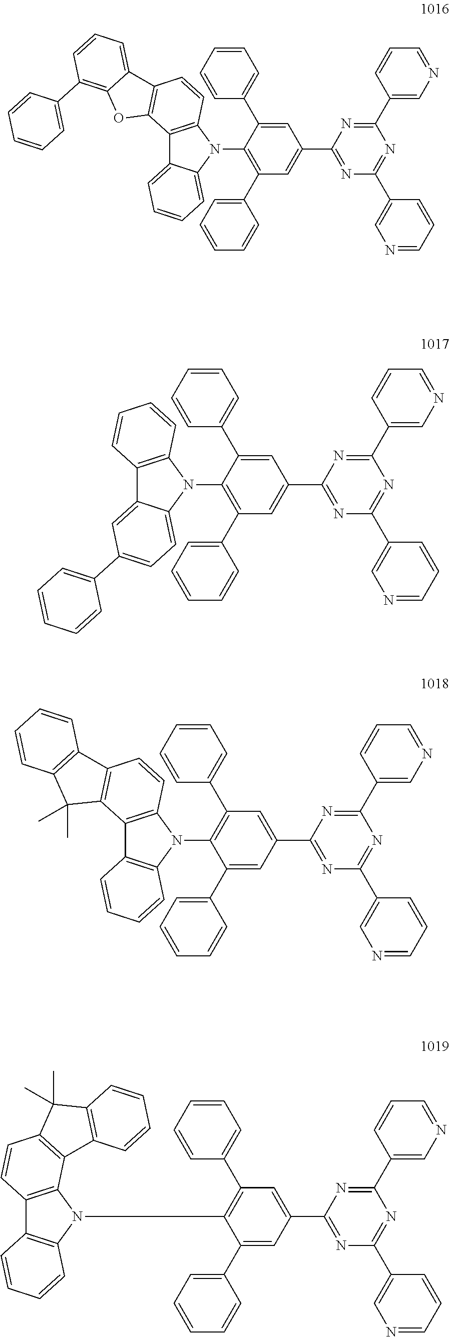 [SMILES: c1ccc(-c2cc(-c3nc(-c4cccnc4)nc(-c4cccnc4)n3)cc(-c3ccccc3)c2-n2c3ccccc3c3c4oc5c(-c6ccccc6)cccc5c4ccc32)cc1 ; c1ccc(-c2ccc3c(c2)c2ccccc2n3-c2c(-c3ccccc3)cc(-c3nc(-c4cccnc4)nc(-c4cccnc4)n3)cc2-c2ccccc2)cc1 ; CC1(C)c2ccccc2-c2ccc3c(c21)c1ccccc1n3-c1c(-c2ccccc2)cc(-c2nc(-c3cccnc3)nc(-c3cccnc3)n2)cc1-c1ccccc1 ; CC1(C)c2ccccc2-c2c1ccc1c3ccccc3n(-c3c(-c4ccccc4)cc(-c4nc(-c5cccnc5)nc(-c5cccnc5)n4)cc3-c3ccccc3)c21]

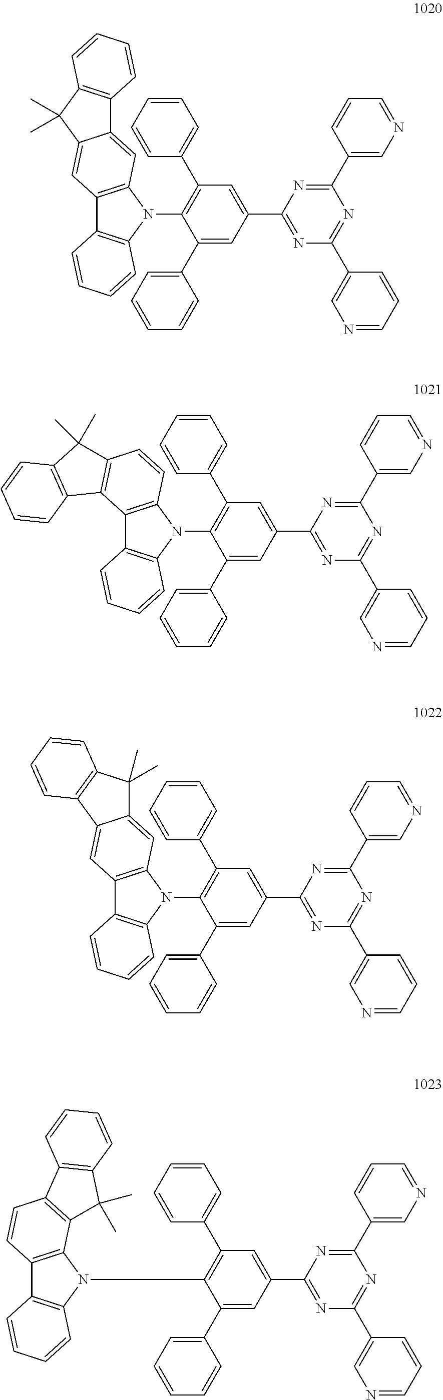 [SMILES: CC1(C)c2ccccc2-c2cc3c(cc21)c1ccccc1n3-c1c(-c2ccccc2)cc(-c2nc(-c3cccnc3)nc(-c3cccnc3)n2)cc1-c1ccccc1 ; CC1(C)c2ccccc2-c2c1ccc1c2c2ccccc2n1-c1c(-c2ccccc2)cc(-c2nc(-c3cccnc3)nc(-c3cccnc3)n2)cc1-c1ccccc1 ; CC1(C)c2ccccc2-c2cc3c4ccccc4n(-c4c(-c5ccccc5)cc(-c5nc(-c6cccnc6)nc(-c6cccnc6)n5)cc4-c4ccccc4)c3cc21 ; CC1(C)c2ccccc2-c2ccc3c4ccccc4n(-c4c(-c5ccccc5)cc(-c5nc(-c6cccnc6)nc(-c6cccnc6)n5)cc4-c4ccccc4)c3c21]

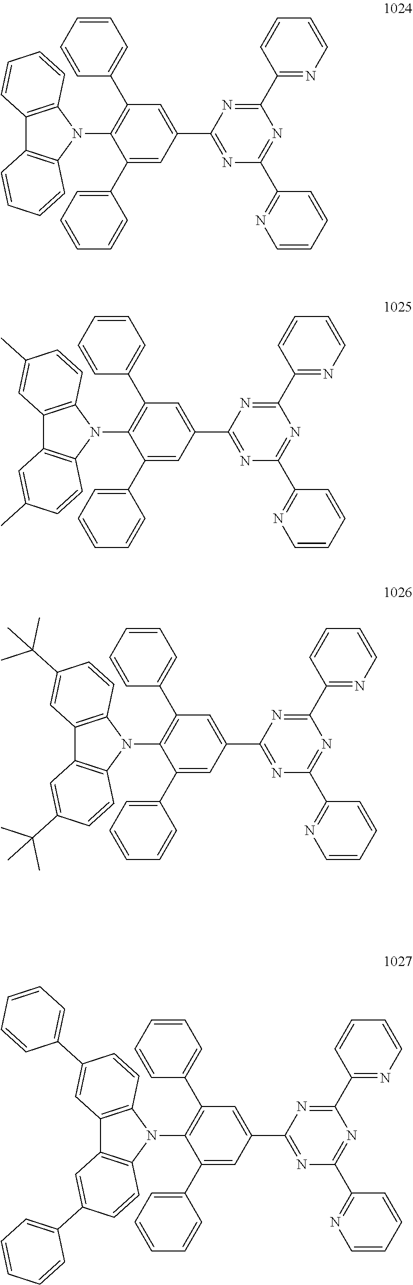 [SMILES: c1ccc(-c2cc(-c3nc(-c4ccccn4)nc(-c4ccccn4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccccn3)nc(-c3ccccn3)n2)cc1-c1ccccc1 ; CC(C)(C)c1ccc2c(c1)c1cc(C(C)(C)C)ccc1n2-c1c(-c2ccccc2)cc(-c2nc(-c3ccccn3)nc(-c3ccccn3)n2)cc1-c1ccccc1 ; c1ccc(-c2ccc3c(c2)c2cc(-c4ccccc4)ccc2n3-c2c(-c3ccccc3)cc(-c3nc(-c4ccccn4)nc(-c4ccccn4)n3)cc2-c2ccccc2)cc1]

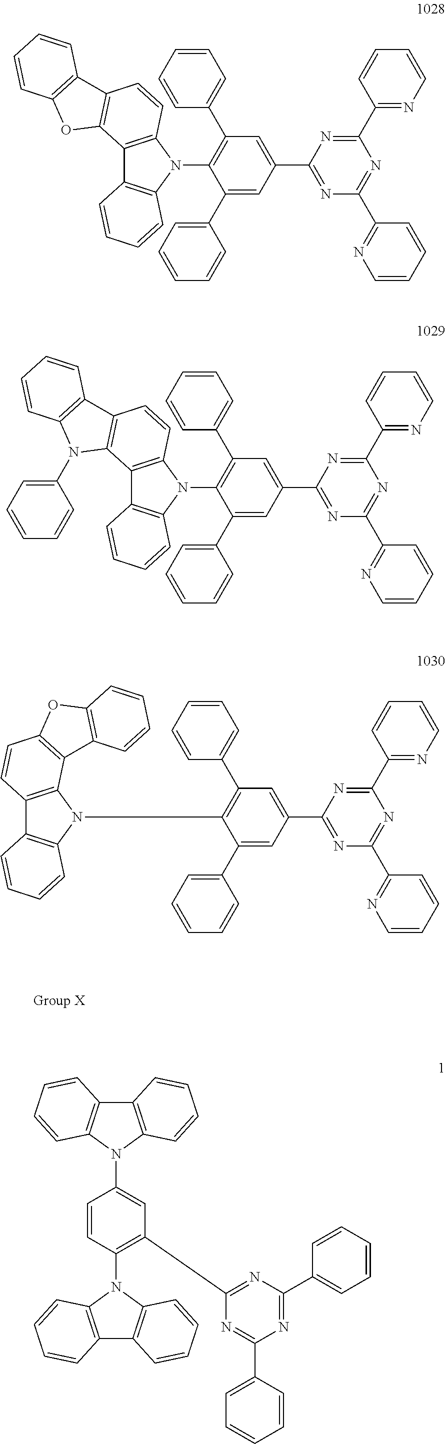 [SMILES: c1ccc(-c2cc(-c3nc(-c4ccccn4)nc(-c4ccccn4)n3)cc(-c3ccccc3)c2-n2c3ccccc3c3c4oc5ccccc5c4ccc32)cc1 ; c1ccc(-c2cc(-c3nc(-c4ccccn4)nc(-c4ccccn4)n3)cc(-c3ccccc3)c2-n2c3ccccc3c3c2ccc2c4ccccc4n(-c4ccccc4)c23)cc1 ; c1ccc(-c2cc(-c3nc(-c4ccccn4)nc(-c4ccccn4)n3)cc(-c3ccccc3)c2-n2c3ccccc3c3ccc4oc5ccccc5c4c32)cc1 ; c1ccc(-c2nc(-c3ccccc3)nc(-c3cc(-n4c5ccccc5c5ccccc54)ccc3-n3c4ccccc4c4ccccc43)n2)cc1]

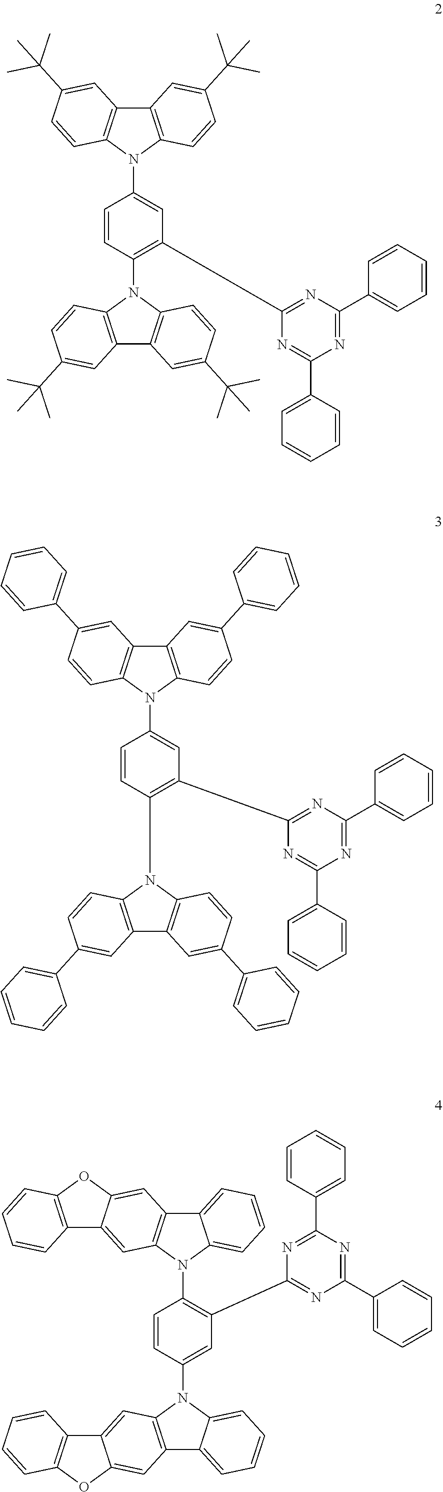 [SMILES: CC(C)(C)c1ccc2c(c1)c1cc(C(C)(C)C)ccc1n2-c1ccc(-n2c3ccc(C(C)(C)C)cc3c3cc(C(C)(C)C)ccc32)c(-c2nc(-c3ccccc3)nc(-c3ccccc3)n2)c1 ; c1ccc(-c2ccc3c(c2)c2cc(-c4ccccc4)ccc2n3-c2ccc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2nc(-c3ccccc3)nc(-c3cc(-n4c5ccccc5c5cc6oc7ccccc7c6cc54)ccc3-n3c4ccccc4c4cc5oc6ccccc6c5cc43)n2)cc1]

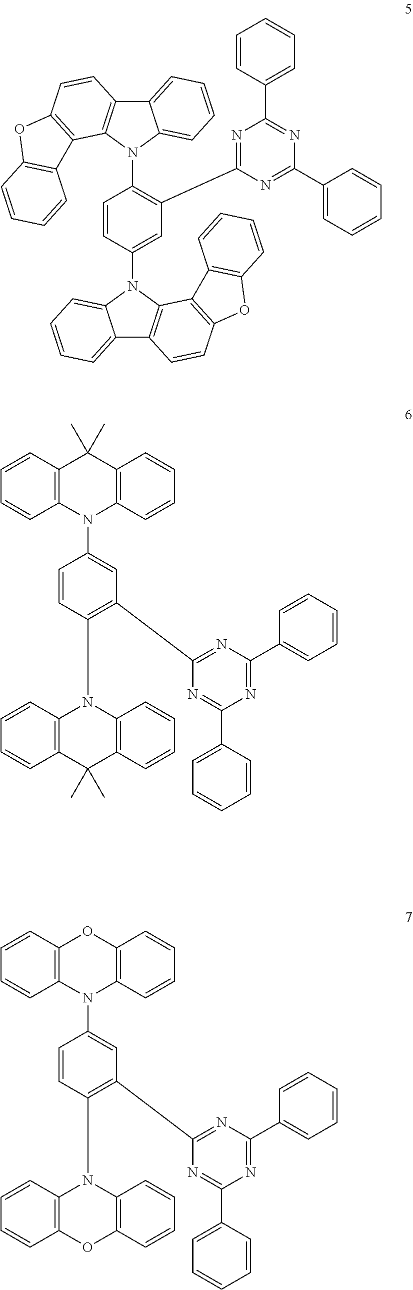 [SMILES: c1ccc(-c2nc(-c3ccccc3)nc(-c3cc(-n4c5ccccc5c5ccc6oc7ccccc7c6c54)ccc3-n3c4ccccc4c4ccc5oc6ccccc6c5c43)n2)cc1 ; CC1(C)c2ccccc2N(c2ccc(N3c4ccccc4C(C)(C)c4ccccc43)c(-c3nc(-c4ccccc4)nc(-c4ccccc4)n3)c2)c2ccccc21 ; c1ccc(-c2nc(-c3ccccc3)nc(-c3cc(N4c5ccccc5Oc5ccccc54)ccc3N3c4ccccc4Oc4ccccc43)n2)cc1]

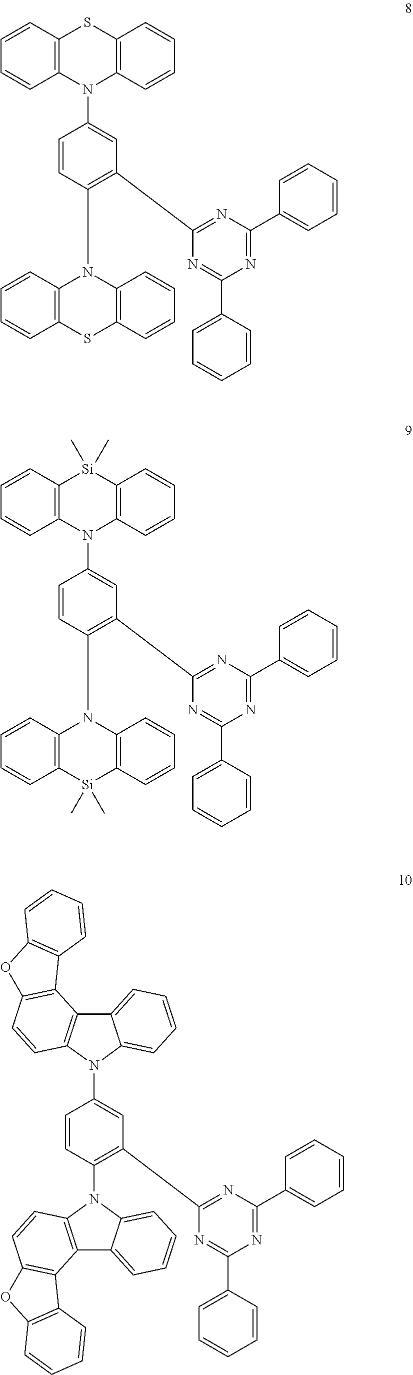 [SMILES: c1ccc(-c2nc(-c3ccccc3)nc(-c3cc(N4c5ccccc5Sc5ccccc54)ccc3N3c4ccccc4Sc4ccccc43)n2)cc1 ; C[Si]1(C)c2ccccc2N(c2ccc(N3c4ccccc4[Si](C)(C)c4ccccc43)c(-c3nc(-c4ccccc4)nc(-c4ccccc4)n3)c2)c2ccccc21 ; c1ccc(-c2nc(-c3ccccc3)nc(-c3cc(-n4c5ccccc5c5c6c(ccc54)oc4ccccc46)ccc3-n3c4ccccc4c4c5c(ccc43)oc3ccccc35)n2)cc1]

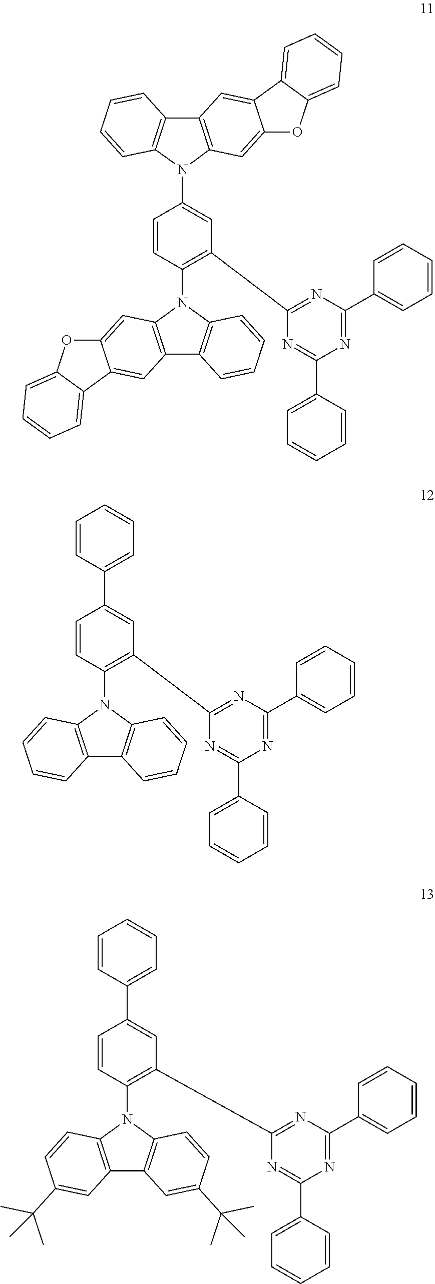 [SMILES: c1ccc(-c2nc(-c3ccccc3)nc(-c3cc(-n4c5ccccc5c5cc6c(cc54)oc4ccccc46)ccc3-n3c4ccccc4c4cc5c(cc43)oc3ccccc35)n2)cc1 ; c1ccc(-c2ccc(-n3c4ccccc4c4ccccc43)c(-c3nc(-c4ccccc4)nc(-c4ccccc4)n3)c2)cc1 ; CC(C)(C)c1ccc2c(c1)c1cc(C(C)(C)C)ccc1n2-c1ccc(-c2ccccc2)cc1-c1nc(-c2ccccc2)nc(-c2ccccc2)n1]

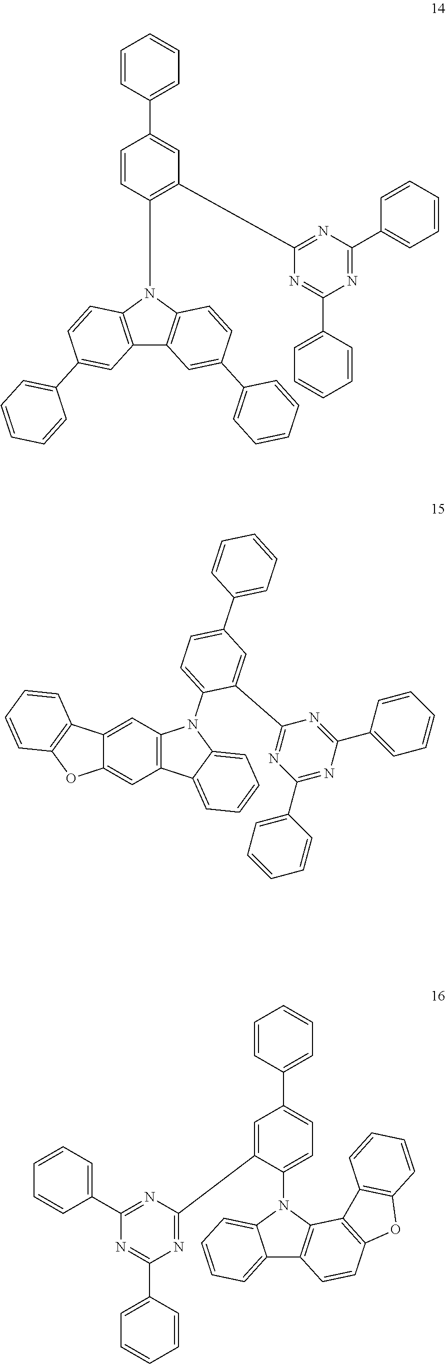 [SMILES: c1ccc(-c2ccc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2ccc(-n3c4ccccc4c4cc5oc6ccccc6c5cc43)c(-c3nc(-c4ccccc4)nc(-c4ccccc4)n3)c2)cc1 ; c1ccc(-c2ccc(-n3c4ccccc4c4ccc5oc6ccccc6c5c43)c(-c3nc(-c4ccccc4)nc(-c4ccccc4)n3)c2)cc1]

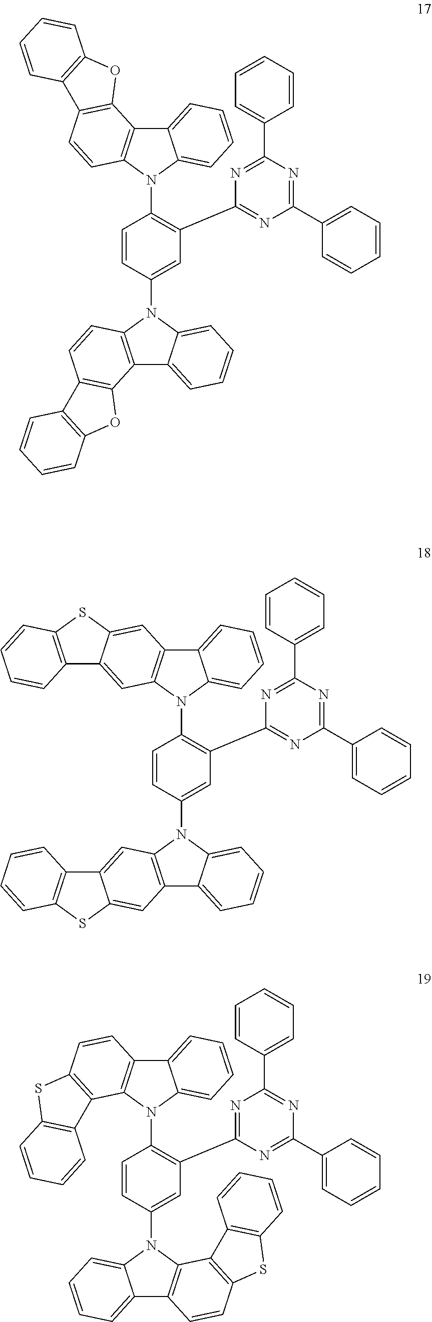 [SMILES: c1ccc(-c2nc(-c3ccccc3)nc(-c3cc(-n4c5ccccc5c5c6oc7ccccc7c6ccc54)ccc3-n3c4ccccc4c4c5oc6ccccc6c5ccc43)n2)cc1 ; c1ccc(-c2nc(-c3ccccc3)nc(-c3cc(-n4c5ccccc5c5cc6sc7ccccc7c6cc54)ccc3-n3c4ccccc4c4cc5sc6ccccc6c5cc43)n2)cc1 ; c1ccc(-c2nc(-c3ccccc3)nc(-c3cc(-n4c5ccccc5c5ccc6sc7ccccc7c6c54)ccc3-n3c4ccccc4c4ccc5sc6ccccc6c5c43)n2)cc1]

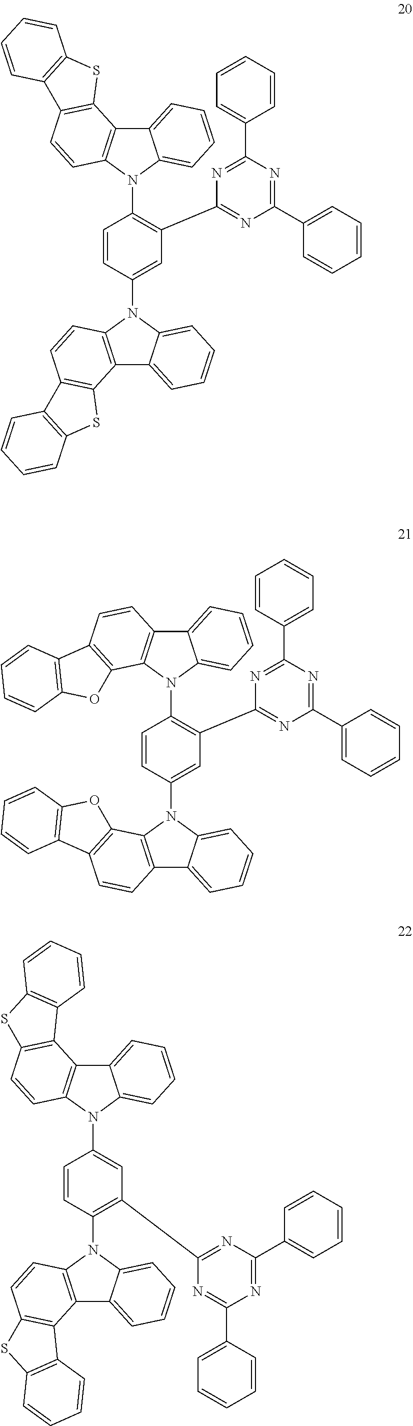 [SMILES: c1ccc(-c2nc(-c3ccccc3)nc(-c3cc(-n4c5ccccc5c5c6sc7ccccc7c6ccc54)ccc3-n3c4ccccc4c4c5sc6ccccc6c5ccc43)n2)cc1 ; c1ccc(-c2nc(-c3ccccc3)nc(-c3cc(-n4c5ccccc5c5ccc6c7ccccc7oc6c54)ccc3-n3c4ccccc4c4ccc5c6ccccc6oc5c43)n2)cc1 ; c1ccc(-c2nc(-c3ccccc3)nc(-c3cc(-n4c5ccccc5c5c6c(ccc54)sc4ccccc46)ccc3-n3c4ccccc4c4c5c(ccc43)sc3ccccc35)n2)cc1]

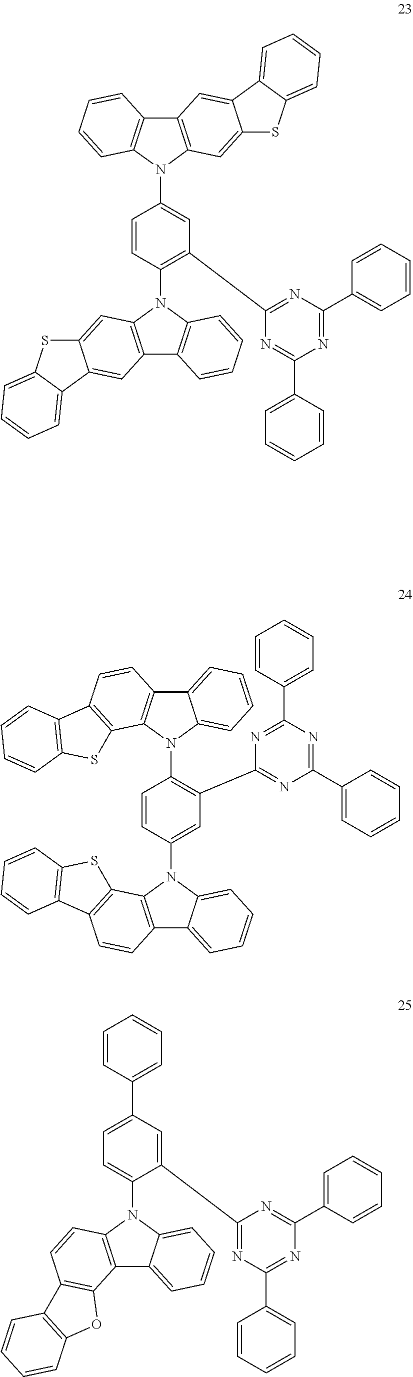 [SMILES: c1ccc(-c2nc(-c3ccccc3)nc(-c3cc(-n4c5ccccc5c5cc6c(cc54)sc4ccccc46)ccc3-n3c4ccccc4c4cc5c(cc43)sc3ccccc35)n2)cc1 ; c1ccc(-c2nc(-c3ccccc3)nc(-c3cc(-n4c5ccccc5c5ccc6c7ccccc7sc6c54)ccc3-n3c4ccccc4c4ccc5c6ccccc6sc5c43)n2)cc1 ; c1ccc(-c2ccc(-n3c4ccccc4c4c5oc6ccccc6c5ccc43)c(-c3nc(-c4ccccc4)nc(-c4ccccc4)n3)c2)cc1]

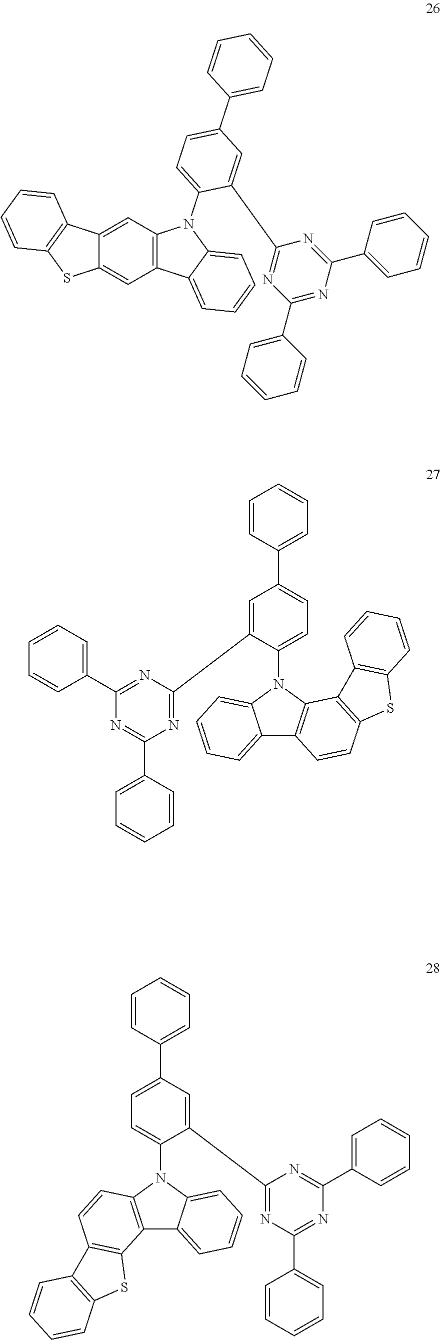 [SMILES: c1ccc(-c2ccc(-n3c4ccccc4c4cc5sc6ccccc6c5cc43)c(-c3nc(-c4ccccc4)nc(-c4ccccc4)n3)c2)cc1 ; c1ccc(-c2ccc(-n3c4ccccc4c4ccc5sc6ccccc6c5c43)c(-c3nc(-c4ccccc4)nc(-c4ccccc4)n3)c2)cc1 ; c1ccc(-c2ccc(-n3c4ccccc4c4c5sc6ccccc6c5ccc43)c(-c3nc(-c4ccccc4)nc(-c4ccccc4)n3)c2)cc1]

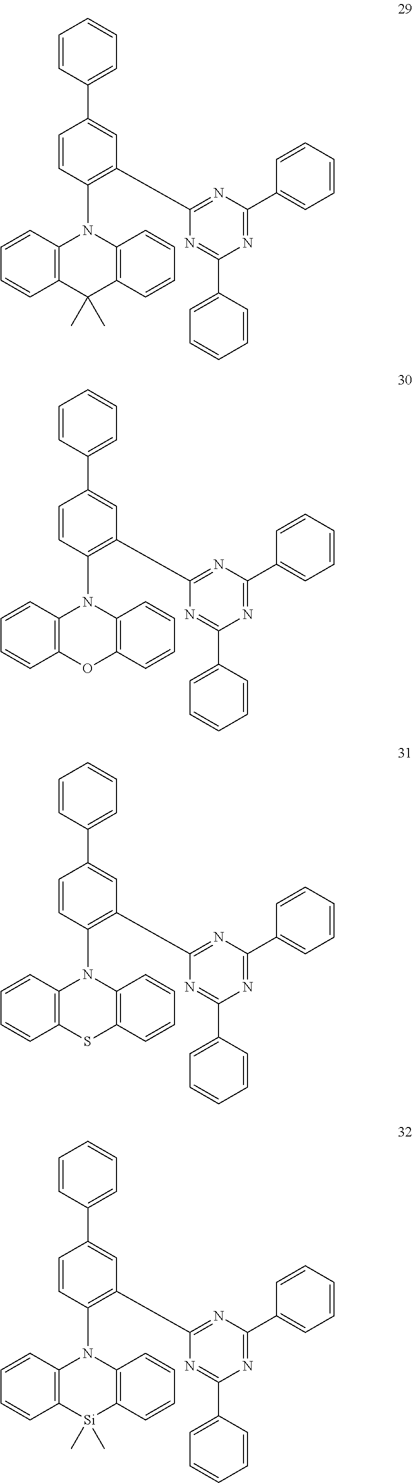 [SMILES: CC1(C)c2ccccc2N(c2ccc(-c3ccccc3)cc2-c2nc(-c3ccccc3)nc(-c3ccccc3)n2)c2ccccc21 ; c1ccc(-c2ccc(N3c4ccccc4Oc4ccccc43)c(-c3nc(-c4ccccc4)nc(-c4ccccc4)n3)c2)cc1 ; c1ccc(-c2ccc(N3c4ccccc4Sc4ccccc43)c(-c3nc(-c4ccccc4)nc(-c4ccccc4)n3)c2)cc1 ; C[Si]1(C)c2ccccc2N(c2ccc(-c3ccccc3)cc2-c2nc(-c3ccccc3)nc(-c3ccccc3)n2)c2ccccc21]

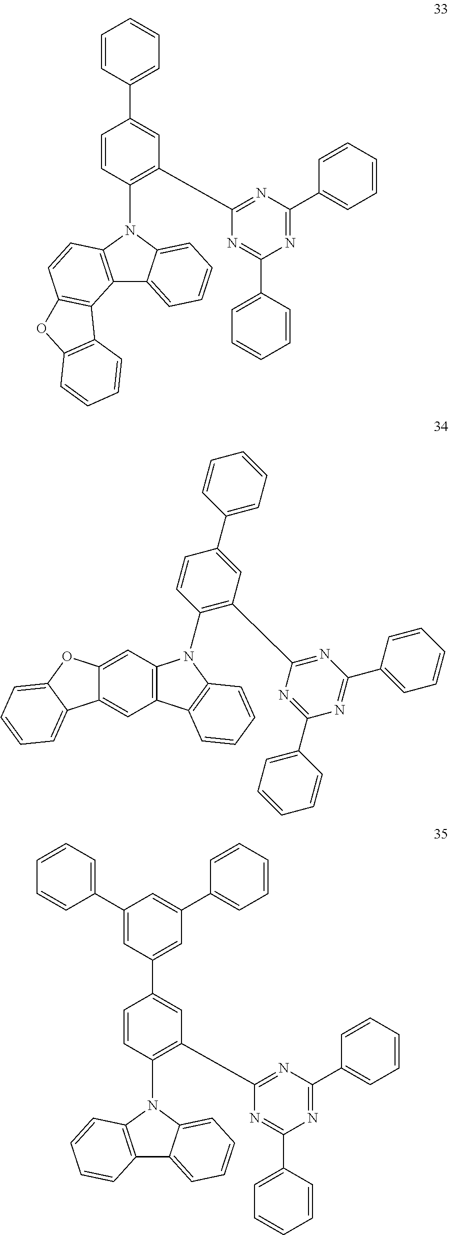 [SMILES: c1ccc(-c2ccc(-n3c4ccccc4c4c5c(ccc43)oc3ccccc35)c(-c3nc(-c4ccccc4)nc(-c4ccccc4)n3)c2)cc1 ; c1ccc(-c2ccc(-n3c4ccccc4c4cc5c(cc43)oc3ccccc35)c(-c3nc(-c4ccccc4)nc(-c4ccccc4)n3)c2)cc1 ; c1ccc(-c2cc(-c3ccccc3)cc(-c3ccc(-n4c5ccccc5c5ccccc54)c(-c4nc(-c5ccccc5)nc(-c5ccccc5)n4)c3)c2)cc1]

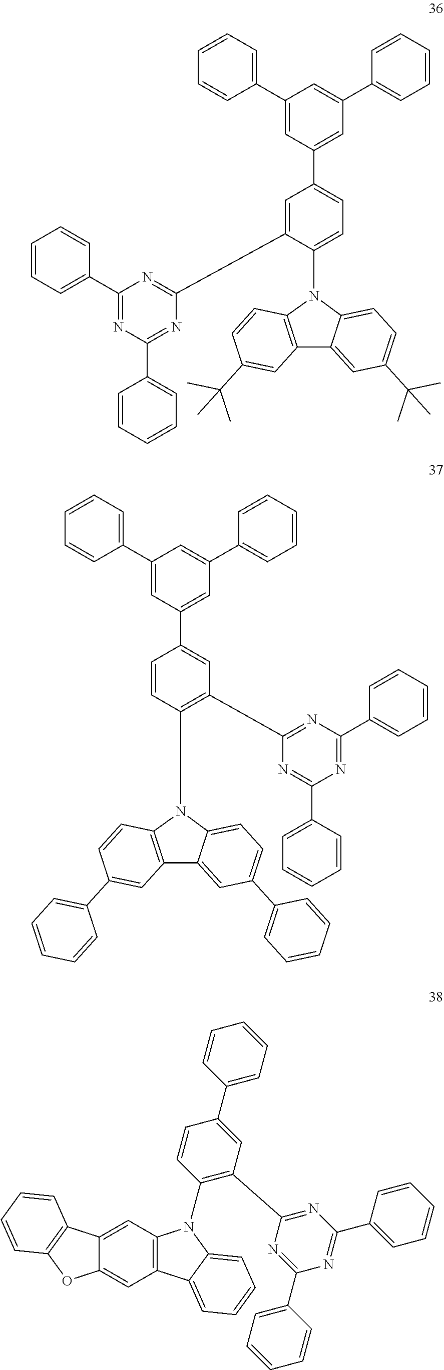 [SMILES: CC(C)(C)c1ccc2c(c1)c1cc(C(C)(C)C)ccc1n2-c1ccc(-c2cc(-c3ccccc3)cc(-c3ccccc3)c2)cc1-c1nc(-c2ccccc2)nc(-c2ccccc2)n1 ; c1ccc(-c2cc(-c3ccccc3)cc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2ccc(-n3c4ccccc4c4cc5oc6ccccc6c5cc43)c(-c3nc(-c4ccccc4)nc(-c4ccccc4)n3)c2)cc1]

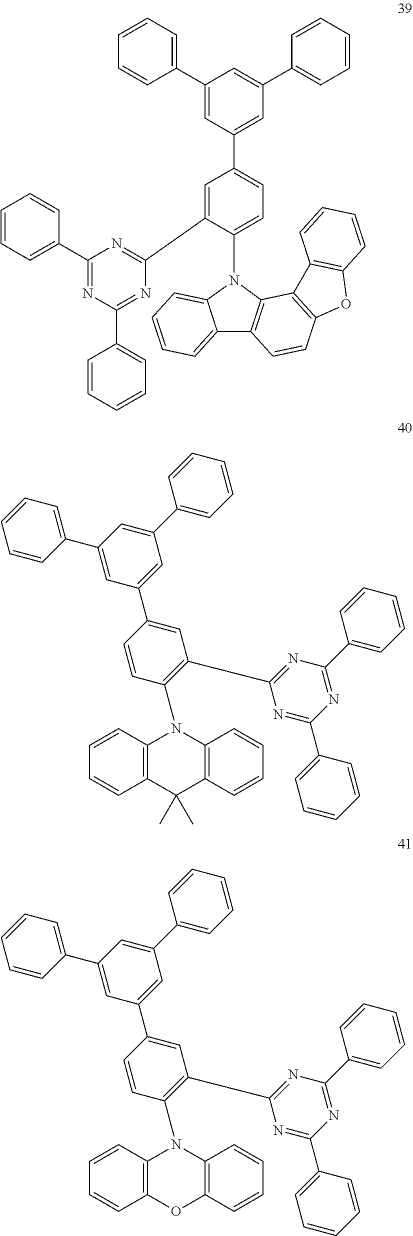

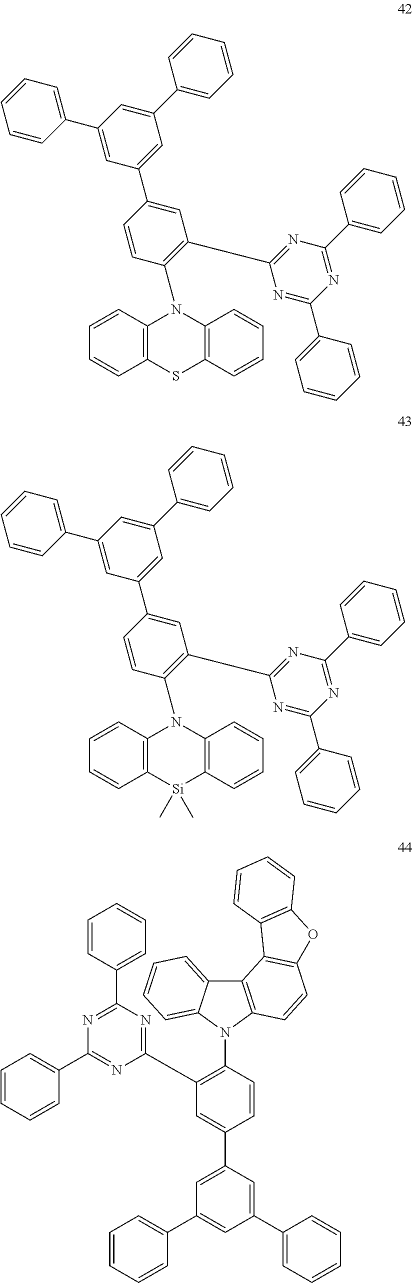 [SMILES: c1ccc(-c2cc(-c3ccccc3)cc(-c3ccc(N4c5ccccc5Sc5ccccc54)c(-c4nc(-c5ccccc5)nc(-c5ccccc5)n4)c3)c2)cc1 ; C[Si]1(C)c2ccccc2N(c2ccc(-c3cc(-c4ccccc4)cc(-c4ccccc4)c3)cc2-c2nc(-c3ccccc3)nc(-c3ccccc3)n2)c2ccccc21 ; c1ccc(-c2cc(-c3ccccc3)cc(-c3ccc(-n4c5ccccc5c5c6c(ccc54)oc4ccccc46)c(-c4nc(-c5ccccc5)nc(-c5ccccc5)n4)c3)c2)cc1]

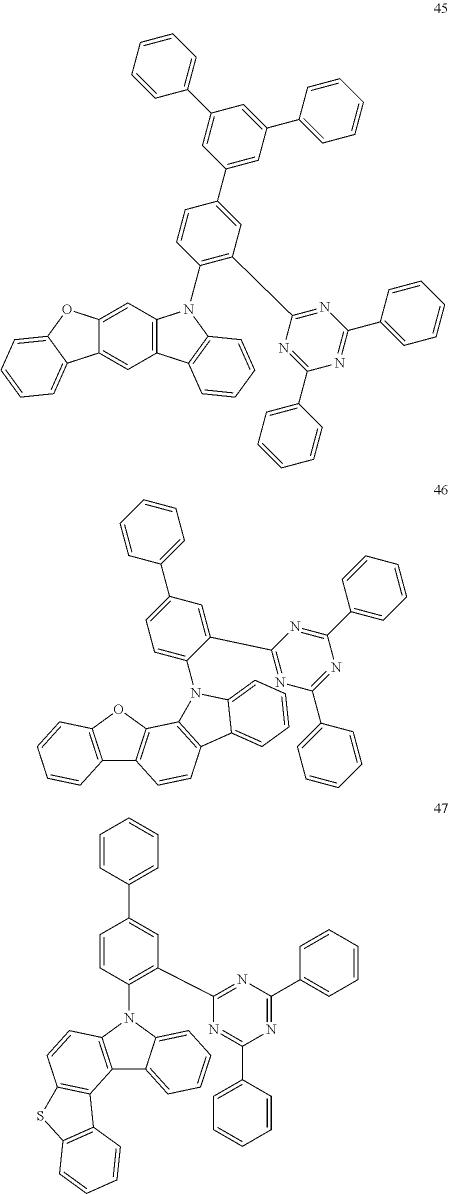

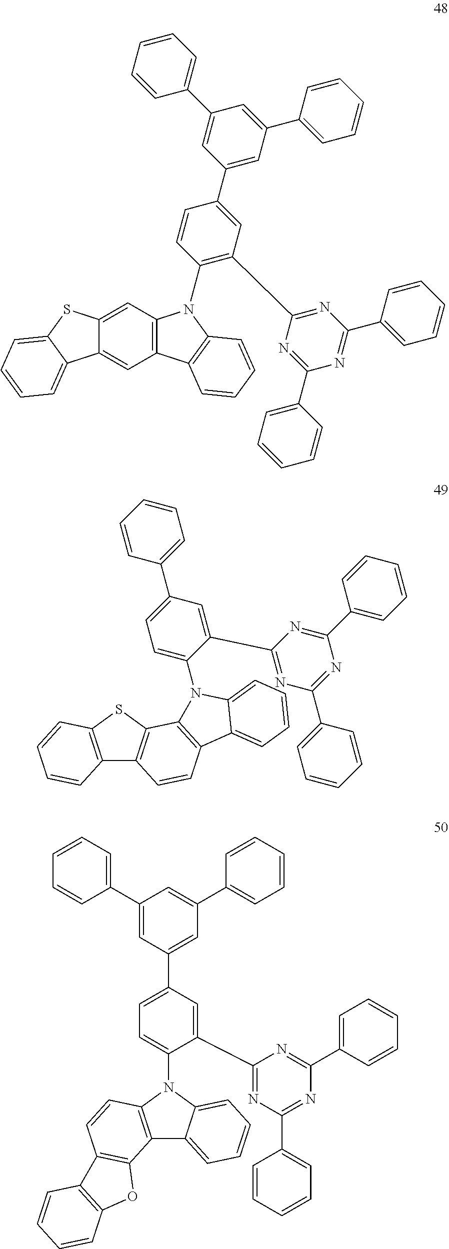 [SMILES: c1ccc(-c2cc(-c3ccccc3)cc(-c3ccc(-n4c5ccccc5c5cc6c(cc54)sc4ccccc46)c(-c4nc(-c5ccccc5)nc(-c5ccccc5)n4)c3)c2)cc1 ; c1ccc(-c2ccc(-n3c4ccccc4c4ccc5c6ccccc6sc5c43)c(-c3nc(-c4ccccc4)nc(-c4ccccc4)n3)c2)cc1 ; c1ccc(-c2cc(-c3ccccc3)cc(-c3ccc(-n4c5ccccc5c5c6oc7ccccc7c6ccc54)c(-c4nc(-c5ccccc5)nc(-c5ccccc5)n4)c3)c2)cc1]

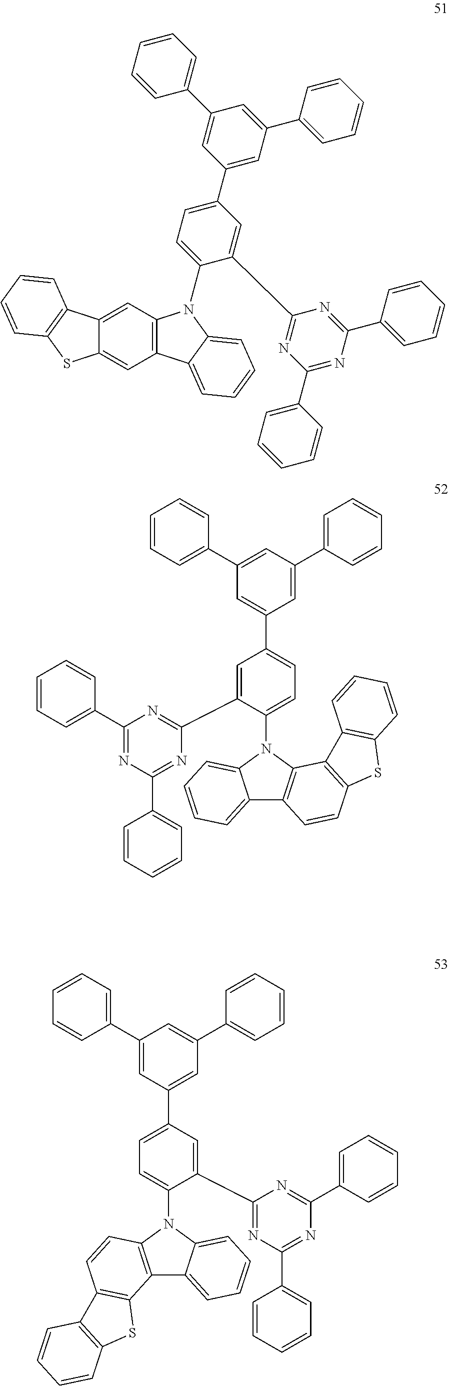 [SMILES: c1ccc(-c2cc(-c3ccccc3)cc(-c3ccc(-n4c5ccccc5c5cc6sc7ccccc7c6cc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2cc(-c3ccccc3)cc(-c3ccc(-n4c5ccccc5c5c6sc7ccccc7c6ccc54)c(-c4nc(-c5ccccc5)nc(-c5ccccc5)n4)c3)c2)cc1]

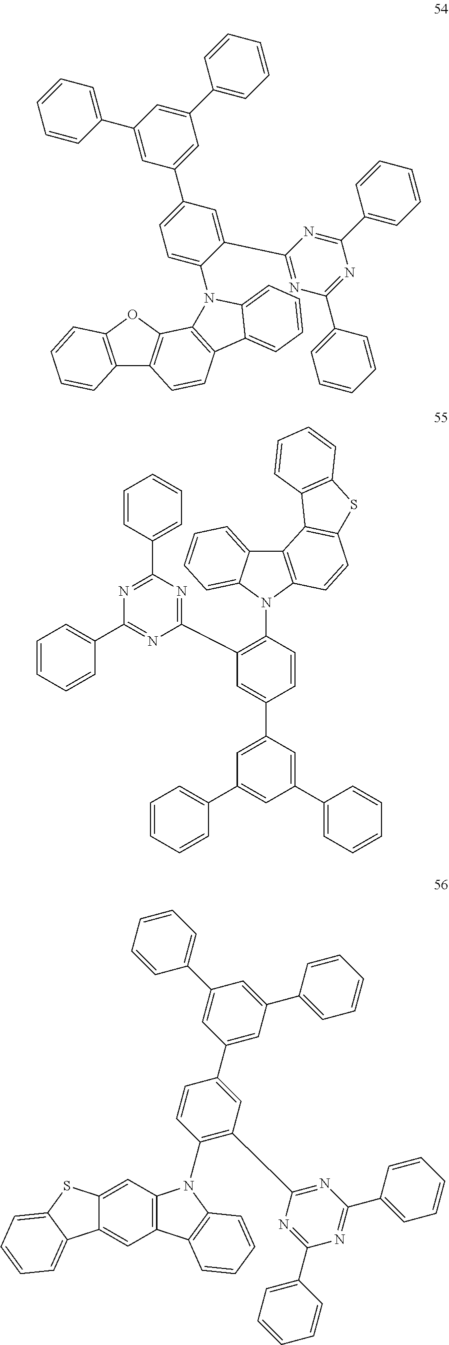

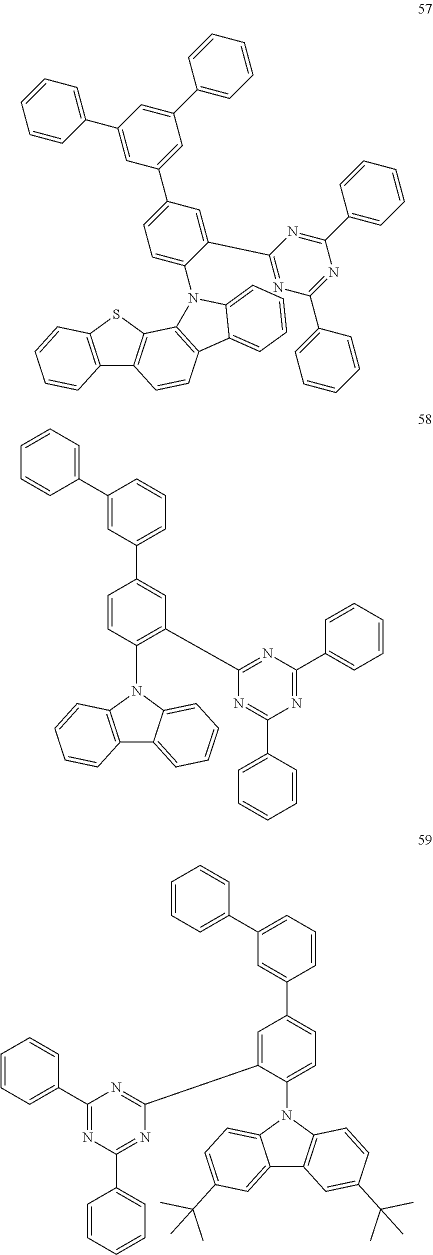

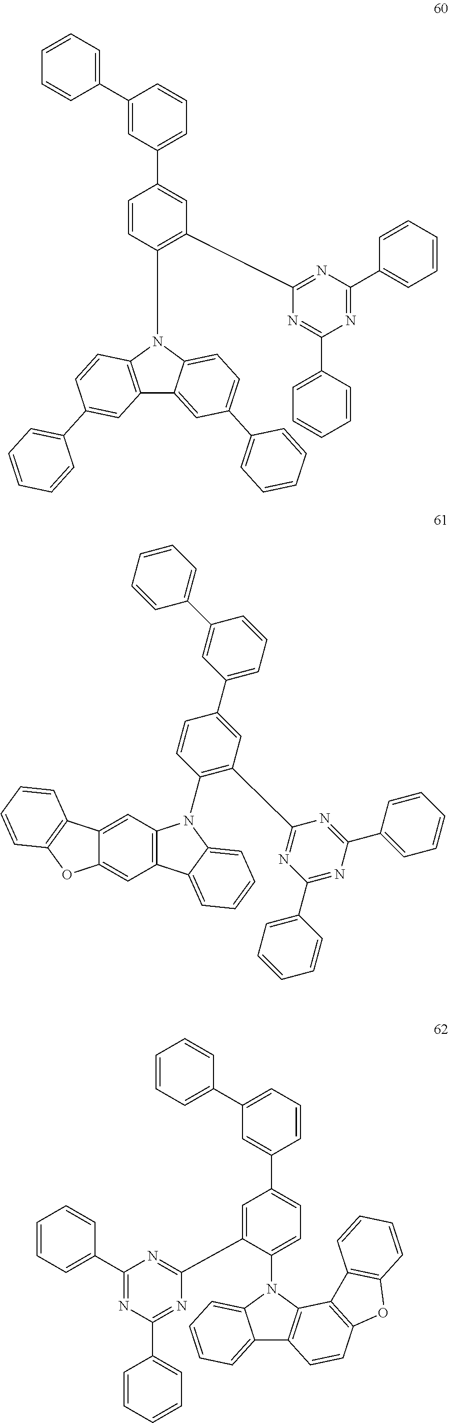 [SMILES: c1ccc(-c2cccc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2cccc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cccc(-c3ccc(-n4c5ccccc5c5ccc6oc7ccccc7c6c54)c(-c4nc(-c5ccccc5)nc(-c5ccccc5)n4)c3)c2)cc1]

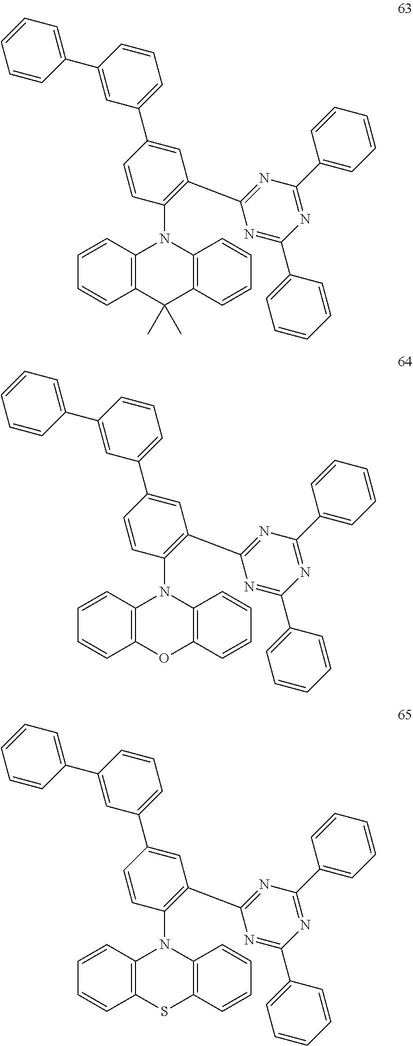 [SMILES: CC1(C)c2ccccc2N(c2ccc(-c3cccc(-c4ccccc4)c3)cc2-c2nc(-c3ccccc3)nc(-c3ccccc3)n2)c2ccccc21 ; c1ccc(-c2cccc(-c3ccc(N4c5ccccc5Oc5ccccc54)c(-c4nc(-c5ccccc5)nc(-c5ccccc5)n4)c3)c2)cc1 ; c1ccc(-c2cccc(-c3ccc(N4c5ccccc5Sc5ccccc54)c(-c4nc(-c5ccccc5)nc(-c5ccccc5)n4)c3)c2)cc1]

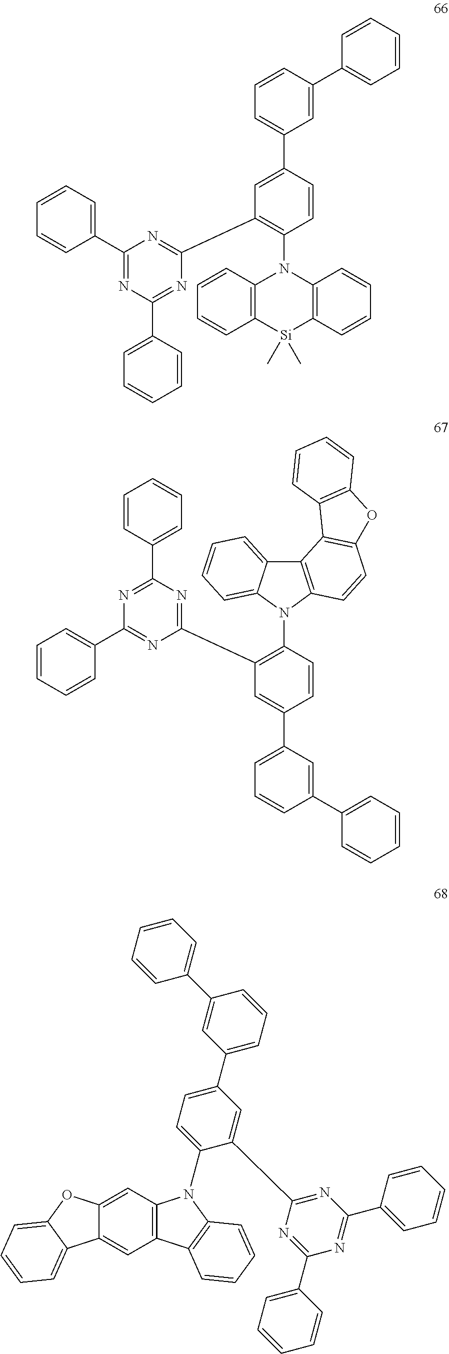 [SMILES: C[Si]1(C)c2ccccc2N(c2ccc(-c3cccc(-c4ccccc4)c3)cc2-c2nc(-c3ccccc3)nc(-c3ccccc3)n2)c2ccccc21 ; c1ccc(-c2cccc(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2cccc(-c3ccc(-n4c5ccccc5c5cc6c(cc54)oc4ccccc46)c(-c4nc(-c5ccccc5)nc(-c5ccccc5)n4)c3)c2)cc1]

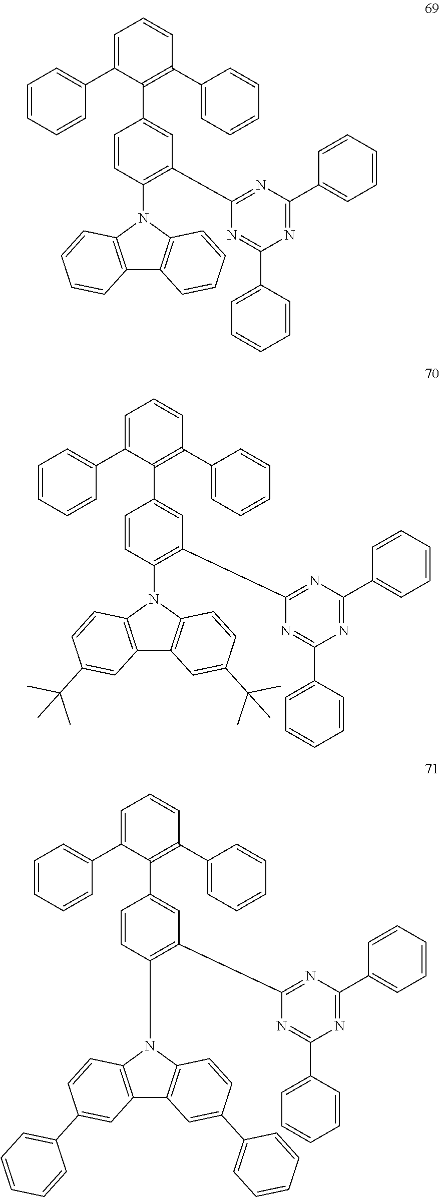 [SMILES: c1ccc(-c2nc(-c3ccccc3)nc(-c3cc(-c4c(-c5ccccc5)cccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2c(-c3ccccc3)cccc2-c2ccccc2)cc1-c1nc(-c2ccccc2)nc(-c2ccccc2)n1 ; c1ccc(-c2ccc3c(c2)c2cc(-c4ccccc4)ccc2n3-c2ccc(-c3c(-c4ccccc4)cccc3-c3ccccc3)cc2-c2nc(-c3ccccc3)nc(-c3ccccc3)n2)cc1]

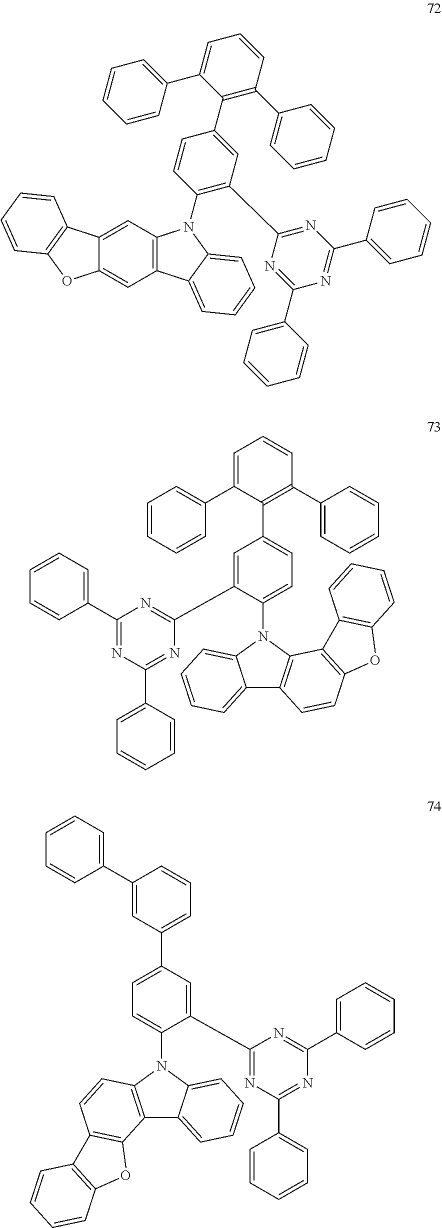 [SMILES: c1ccc(-c2nc(-c3ccccc3)nc(-c3cc(-c4c(-c5ccccc5)cccc4-c4ccccc4)ccc3-n3c4ccccc4c4cc5oc6ccccc6c5cc43)n2)cc1 ; c1ccc(-c2nc(-c3ccccc3)nc(-c3cc(-c4c(-c5ccccc5)cccc4-c4ccccc4)ccc3-n3c4ccccc4c4ccc5oc6ccccc6c5c43)n2)cc1 ; c1ccc(-c2cccc(-c3ccc(-n4c5ccccc5c5c6oc7ccccc7c6ccc54)c(-c4nc(-c5ccccc5)nc(-c5ccccc5)n4)c3)c2)cc1]

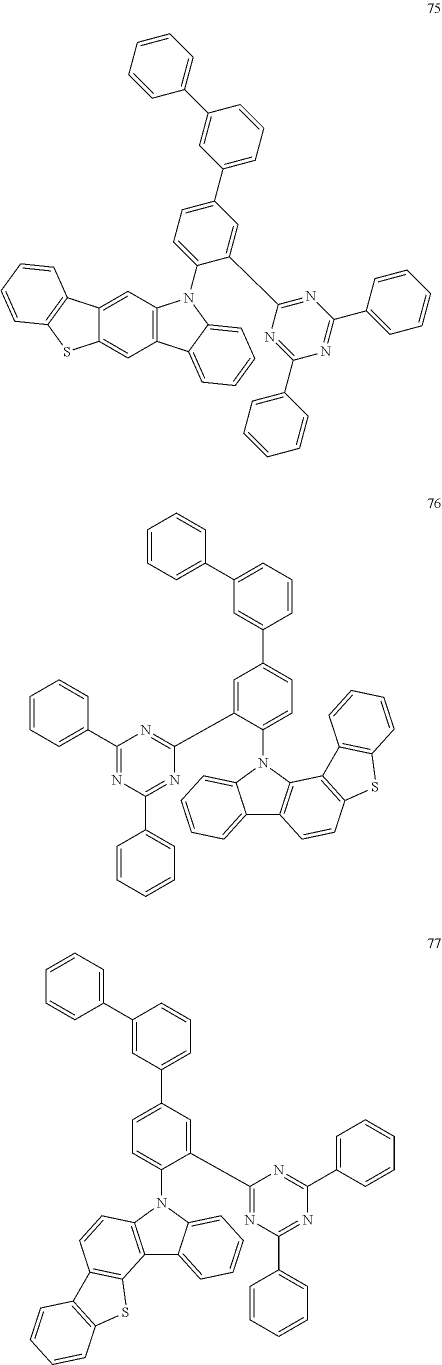 [SMILES: c1ccc(-c2cccc(-c3ccc(-n4c5ccccc5c5cc6sc7ccccc7c6cc54)c(-c4nc(-c5ccccc5)nc(-c5ccccc5)n4)c3)c2)cc1 ; c1ccc(-c2cccc(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2cccc(-c3ccc(-n4c5ccccc5c5c6sc7ccccc7c6ccc54)c(-c4nc(-c5ccccc5)nc(-c5ccccc5)n4)c3)c2)cc1]

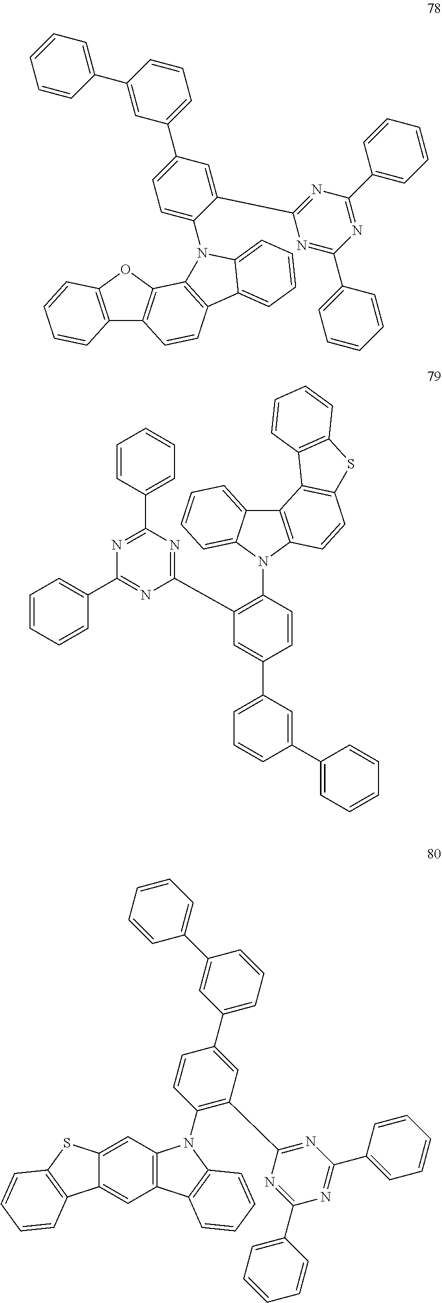 [SMILES: c1ccc(-c2cccc(-c3ccc(-n4c5ccccc5c5ccc6c7ccccc7oc6c54)c(-c4nc(-c5ccccc5)nc(-c5ccccc5)n4)c3)c2)cc1 ; c1ccc(-c2cccc(-c3ccc(-n4c5ccccc5c5c6c(ccc54)sc4ccccc46)c(-c4nc(-c5ccccc5)nc(-c5ccccc5)n4)c3)c2)cc1 ; c1ccc(-c2cccc(-c3ccc(-n4c5ccccc5c5cc6c(cc54)sc4ccccc46)c(-c4nc(-c5ccccc5)nc(-c5ccccc5)n4)c3)c2)cc1]

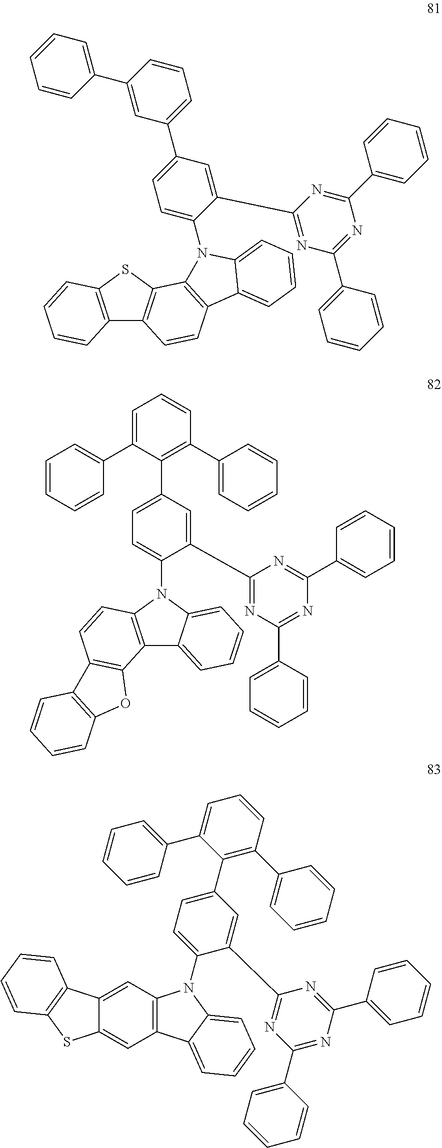 [SMILES: c1ccc(-c2cccc(-c3ccc(-n4c5ccccc5c5ccc6c7ccccc7sc6c54)c(-c4nc(-c5ccccc5)nc(-c5ccccc5)n4)c3)c2)cc1 ; c1ccc(-c2nc(-c3ccccc3)nc(-c3cc(-c4c(-c5ccccc5)cccc4-c4ccccc4)ccc3-n3c4ccccc4c4c5oc6ccccc6c5ccc43)n2)cc1 ; c1ccc(-c2nc(-c3ccccc3)nc(-c3cc(-c4c(-c5ccccc5)cccc4-c4ccccc4)ccc3-n3c4ccccc4c4cc5sc6ccccc6c5cc43)n2)cc1]

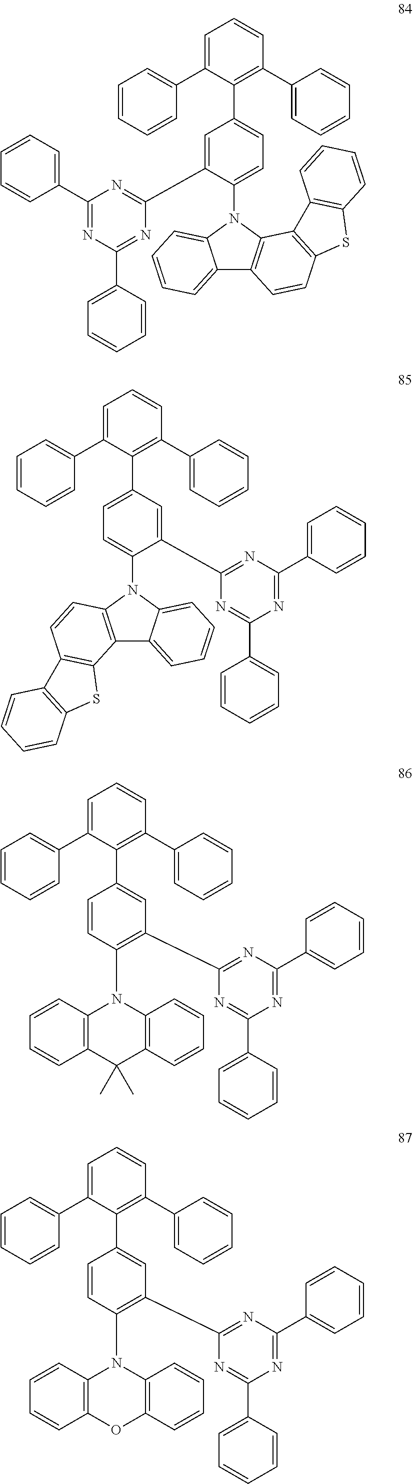

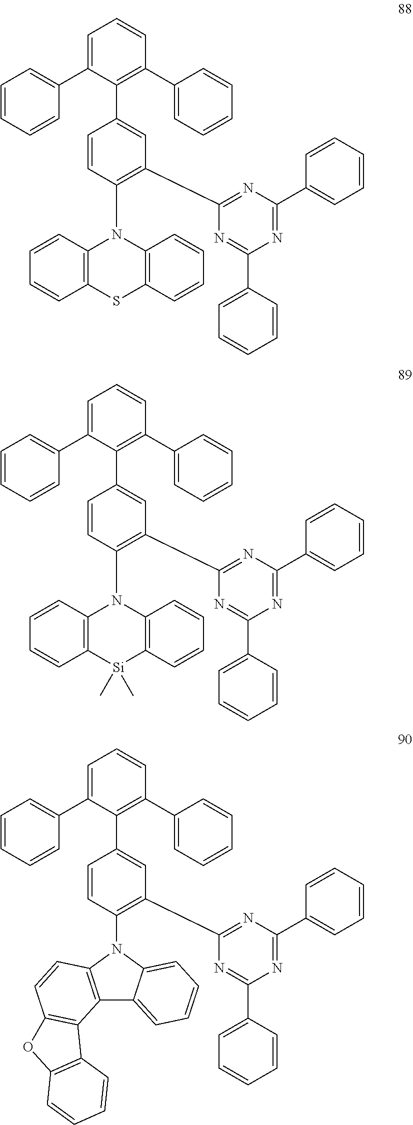 [SMILES: c1ccc(-c2nc(-c3ccccc3)nc(-c3cc(-c4c(-c5ccccc5)cccc4-c4ccccc4)ccc3N3c4ccccc4Sc4ccccc43)n2)cc1 ; C[Si]1(C)c2ccccc2N(c2ccc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)ccc3-n3c4ccccc4c4c5c(ccc43)oc3ccccc35)n2)cc1]

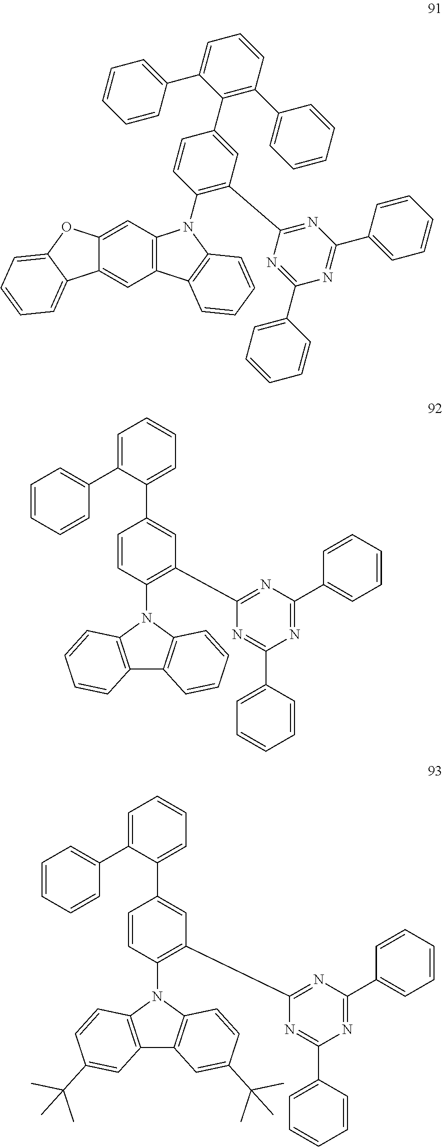 [SMILES: c1ccc(-c2nc(-c3ccccc3)nc(-c3cc(-c4c(-c5ccccc5)cccc4-c4ccccc4)ccc3-n3c4ccccc4c4cc5c(cc43)oc3ccccc35)n2)cc1 ; c1ccc(-c2nc(-c3ccccc3)nc(-c3cc(-c4ccccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2ccccc2-c2ccccc2)cc1-c1nc(-c2ccccc2)nc(-c2ccccc2)n1]

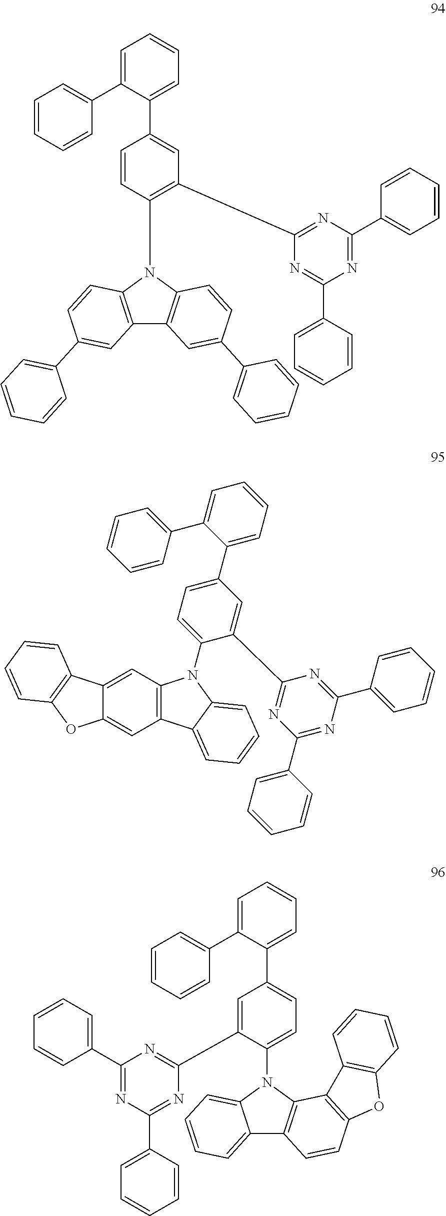 [SMILES: c1ccc(-c2ccc3c(c2)c2cc(-c4ccccc4)ccc2n3-c2ccc(-c3ccccc3-c3ccccc3)cc2-c2nc(-c3ccccc3)nc(-c3ccccc3)n2)cc1 ; c1ccc(-c2nc(-c3ccccc3)nc(-c3cc(-c4ccccc4-c4ccccc4)ccc3-n3c4ccccc4c4cc5oc6ccccc6c5cc43)n2)cc1 ; c1ccc(-c2nc(-c3ccccc3)nc(-c3cc(-c4ccccc4-c4ccccc4)ccc3-n3c4ccccc4c4ccc5oc6ccccc6c5c43)n2)cc1]

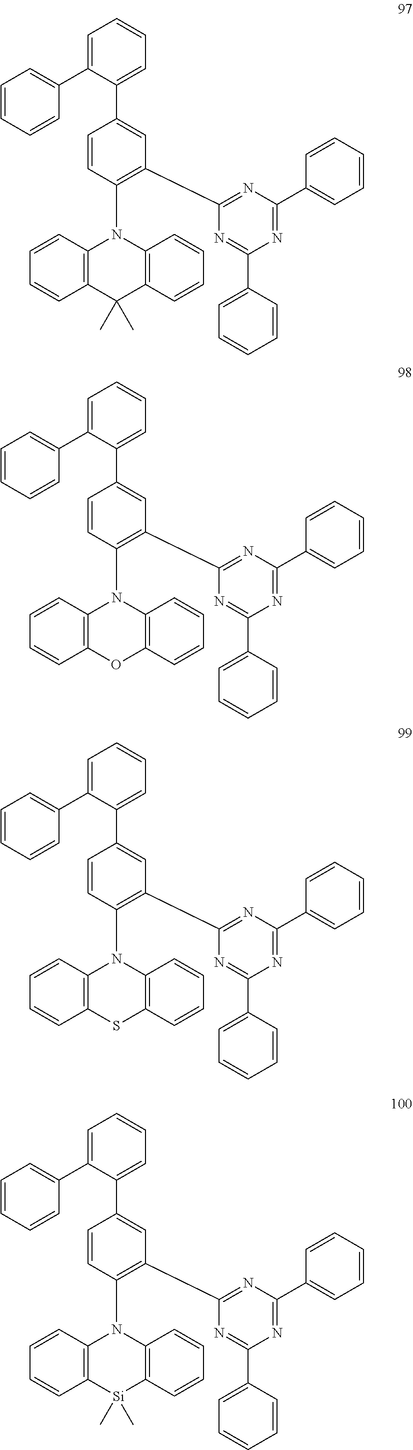 [SMILES: CC1(C)c2ccccc2N(c2ccc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)ccc3N3c4ccccc4Oc4ccccc43)n2)cc1 ; c1ccc(-c2nc(-c3ccccc3)nc(-c3cc(-c4ccccc4-c4ccccc4)ccc3N3c4ccccc4Sc4ccccc43)n2)cc1 ; C[Si]1(C)c2ccccc2N(c2ccc(-c3ccccc3-c3ccccc3)cc2-c2nc(-c3ccccc3)nc(-c3ccccc3)n2)c2ccccc21]

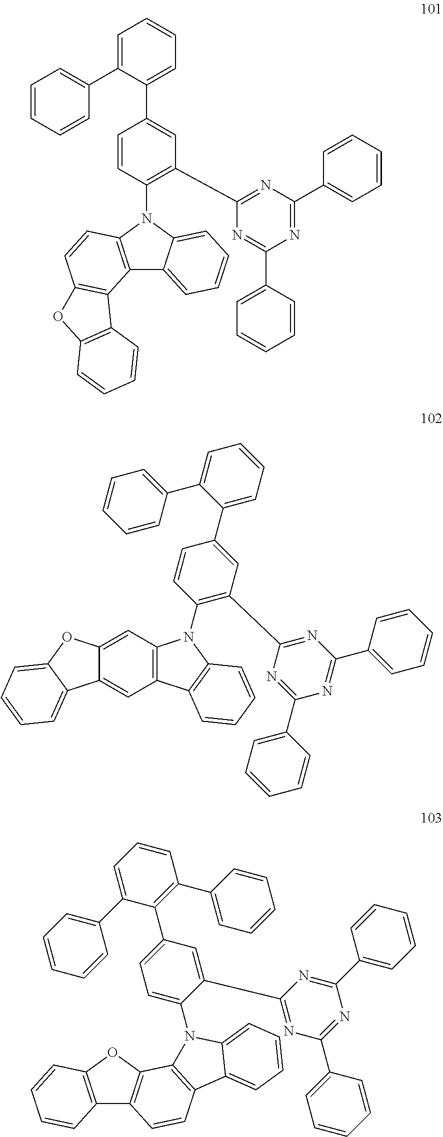 [SMILES: c1ccc(-c2nc(-c3ccccc3)nc(-c3cc(-c4ccccc4-c4ccccc4)ccc3-n3c4ccccc4c4c5c(ccc43)oc3ccccc35)n2)cc1 ; c1ccc(-c2nc(-c3ccccc3)nc(-c3cc(-c4ccccc4-c4ccccc4)ccc3-n3c4ccccc4c4cc5c(cc43)oc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4ccc5c6ccccc6oc5c43)n2)cc1]

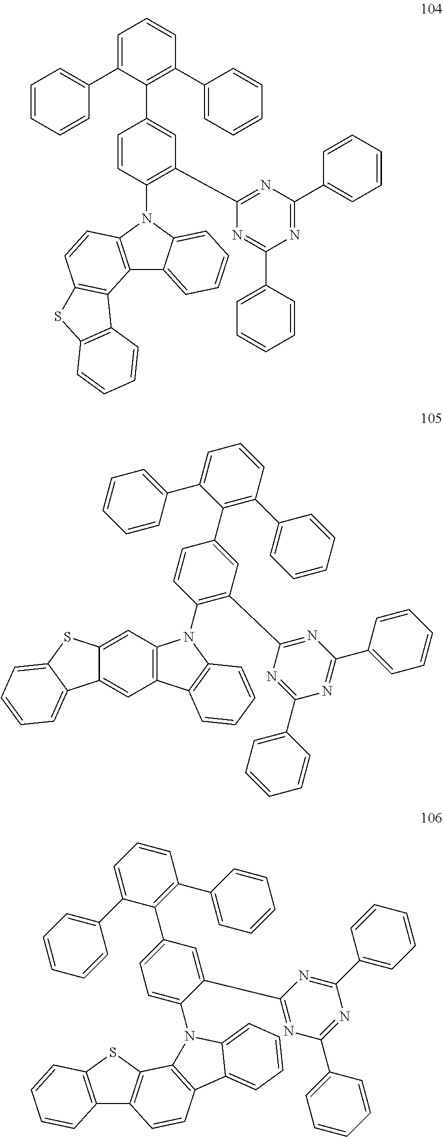 [SMILES: c1ccc(-c2nc(-c3ccccc3)nc(-c3cc(-c4c(-c5ccccc5)cccc4-c4ccccc4)ccc3-n3c4ccccc4c4c5c(ccc43)sc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4cc5c(cc43)sc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4ccc5c6ccccc6sc5c43)n2)cc1]

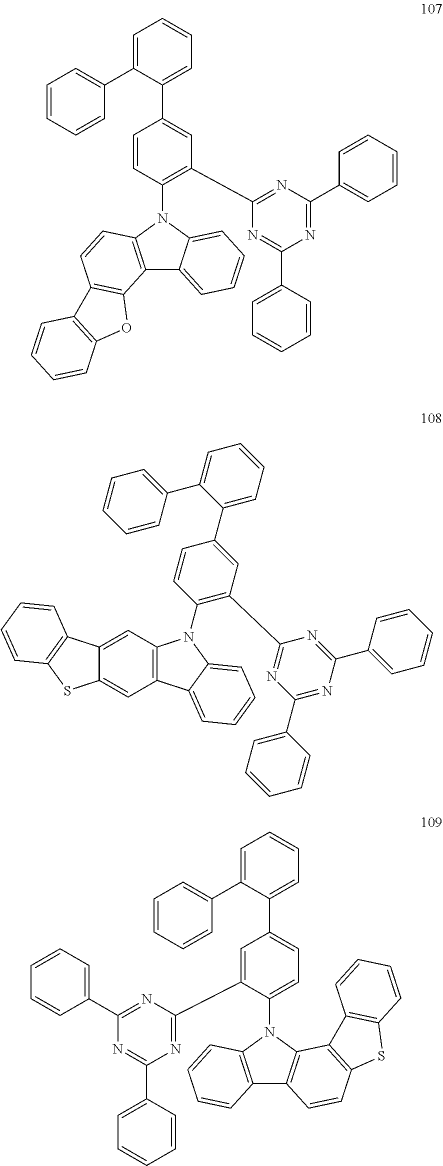

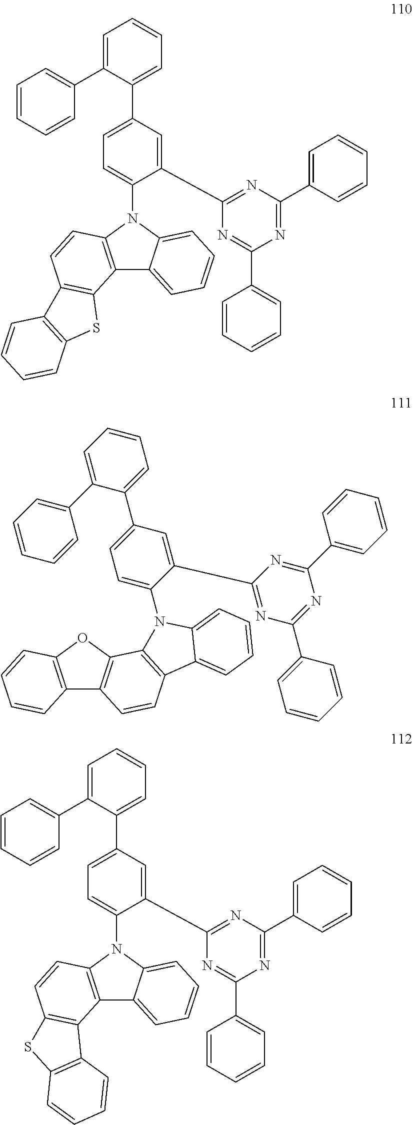

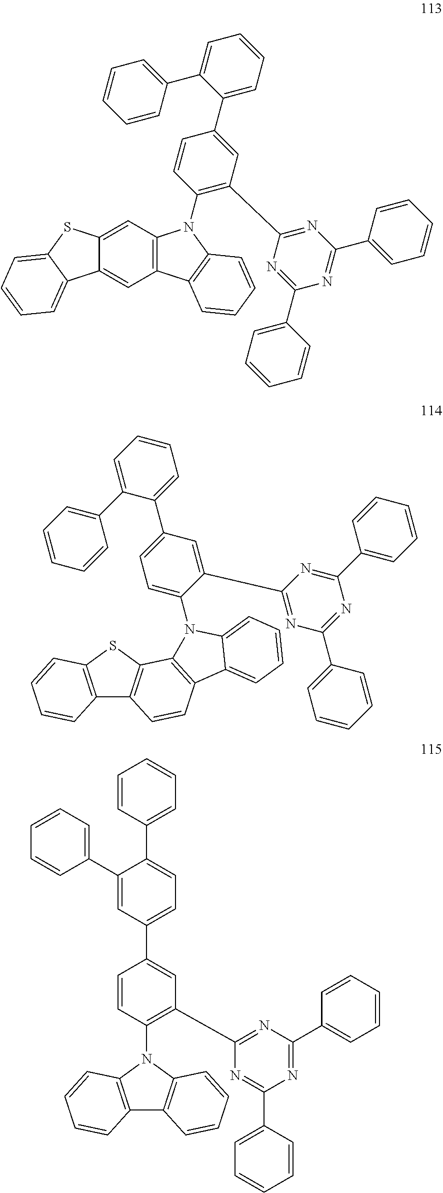

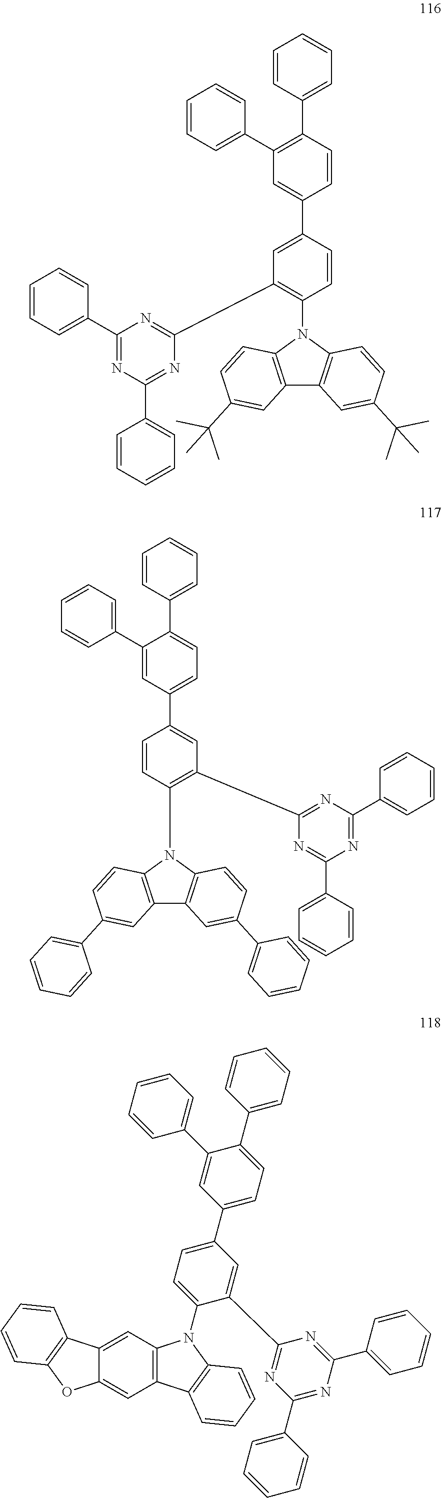 [SMILES: CC(C)(C)c1ccc2c(c1)c1cc(C(C)(C)C)ccc1n2-c1ccc(-c2ccc(-c3ccccc3)c(-c3ccccc3)c2)cc1-c1nc(-c2ccccc2)nc(-c2ccccc2)n1 ; c1ccc(-c2ccc3c(c2)c2cc(-c4ccccc4)ccc2n3-c2ccc(-c3ccc(-c4ccccc4)c(-c4ccccc4)c3)cc2-c2nc(-c3ccccc3)nc(-c3ccccc3)n2)cc1 ; c1ccc(-c2nc(-c3ccccc3)nc(-c3cc(-c4ccc(-c5ccccc5)c(-c5ccccc5)c4)ccc3-n3c4ccccc4c4cc5oc6ccccc6c5cc43)n2)cc1]

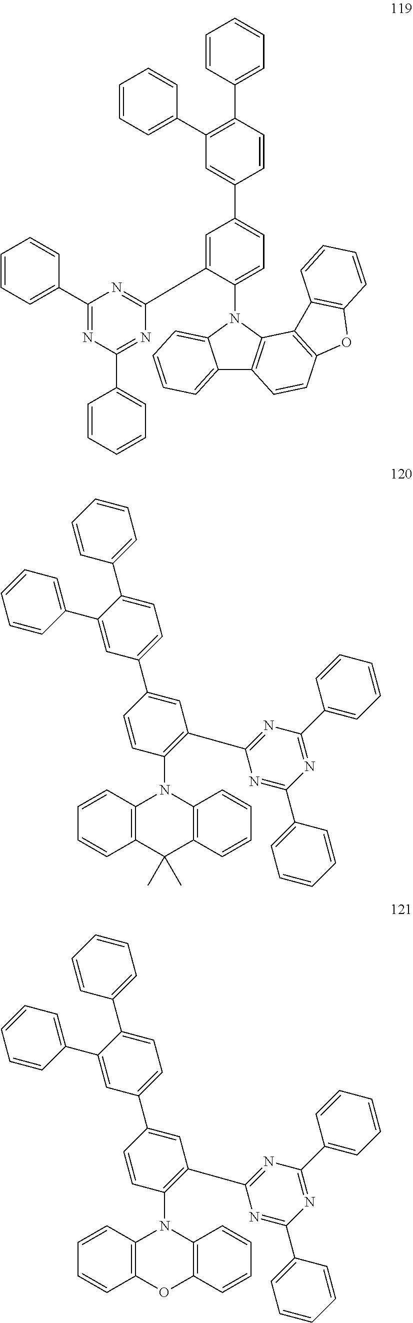 [SMILES: c1ccc(-c2nc(-c3ccccc3)nc(-c3cc(-c4ccc(-c5ccccc5)c(-c5ccccc5)c4)ccc3-n3c4ccccc4c4ccc5oc6ccccc6c5c43)n2)cc1 ; CC1(C)c2ccccc2N(c2ccc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3cc(-c4ccc(-c5ccccc5)c(-c5ccccc5)c4)ccc3N3c4ccccc4Oc4ccccc43)n2)cc1]

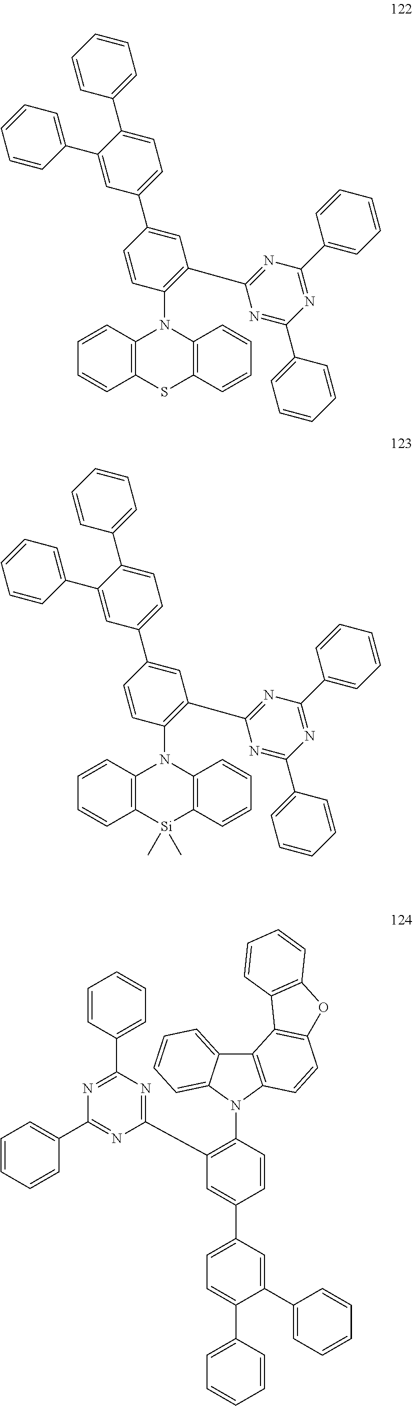 [SMILES: c1ccc(-c2nc(-c3ccccc3)nc(-c3cc(-c4ccc(-c5ccccc5)c(-c5ccccc5)c4)ccc3N3c4ccccc4Sc4ccccc43)n2)cc1 ; C[Si]1(C)c2ccccc2N(c2ccc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3cc(-c4ccc(-c5ccccc5)c(-c5ccccc5)c4)ccc3-n3c4ccccc4c4c5c(ccc43)oc3ccccc35)n2)cc1]

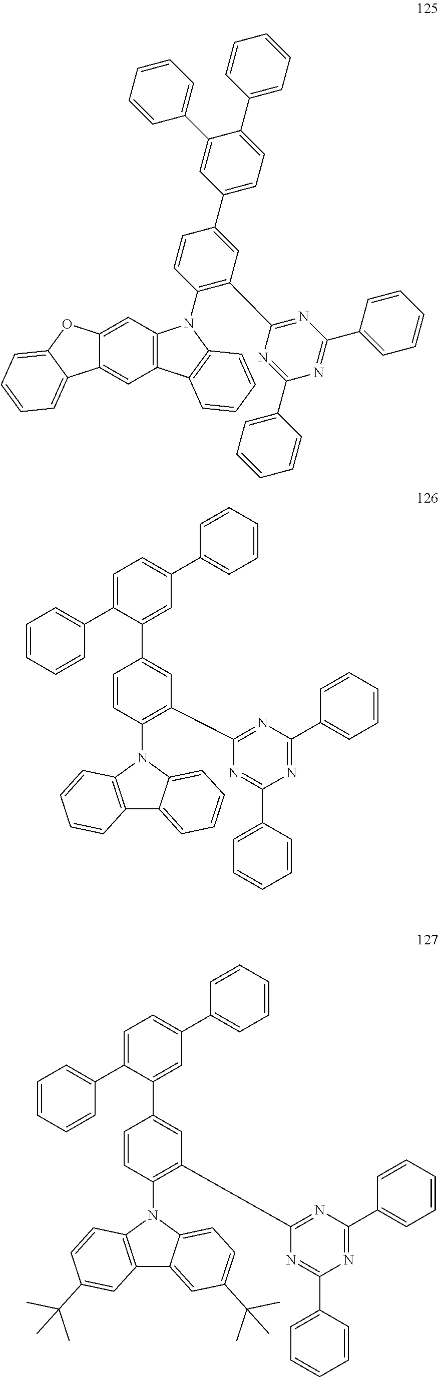

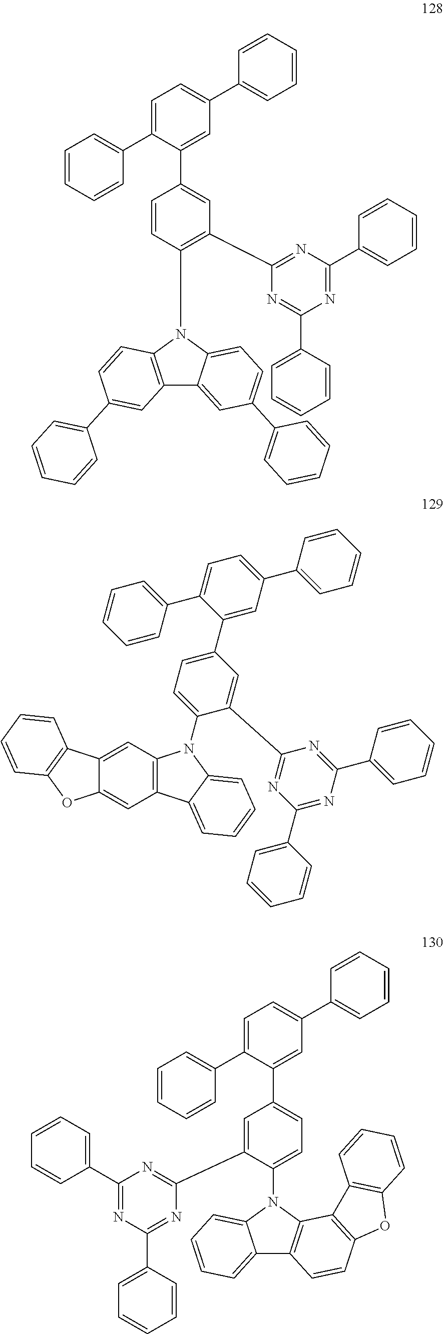 [SMILES: c1ccc(-c2ccc(-c3ccccc3)c(-c3ccc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6oc7ccccc7c6c54)c(-c4nc(-c5ccccc5)nc(-c5ccccc5)n4)c3)c2)cc1]

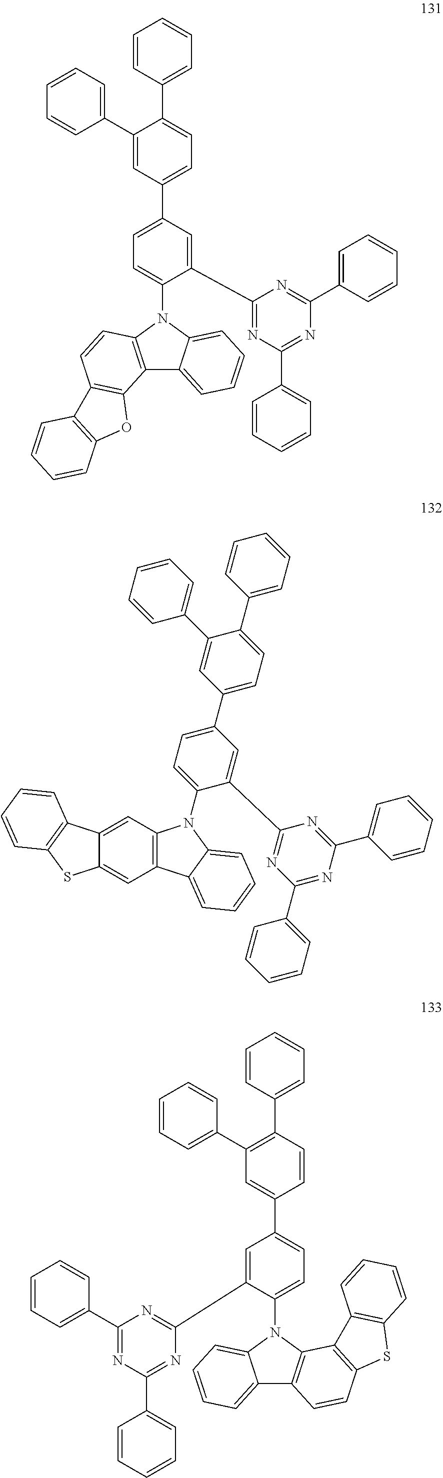 [SMILES: c1ccc(-c2nc(-c3ccccc3)nc(-c3cc(-c4ccc(-c5ccccc5)c(-c5ccccc5)c4)ccc3-n3c4ccccc4c4c5oc6ccccc6c5ccc43)n2)cc1 ; c1ccc(-c2nc(-c3ccccc3)nc(-c3cc(-c4ccc(-c5ccccc5)c(-c5ccccc5)c4)ccc3-n3c4ccccc4c4cc5sc6ccccc6c5cc43)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5sc6ccccc6c5c43)n2)cc1]

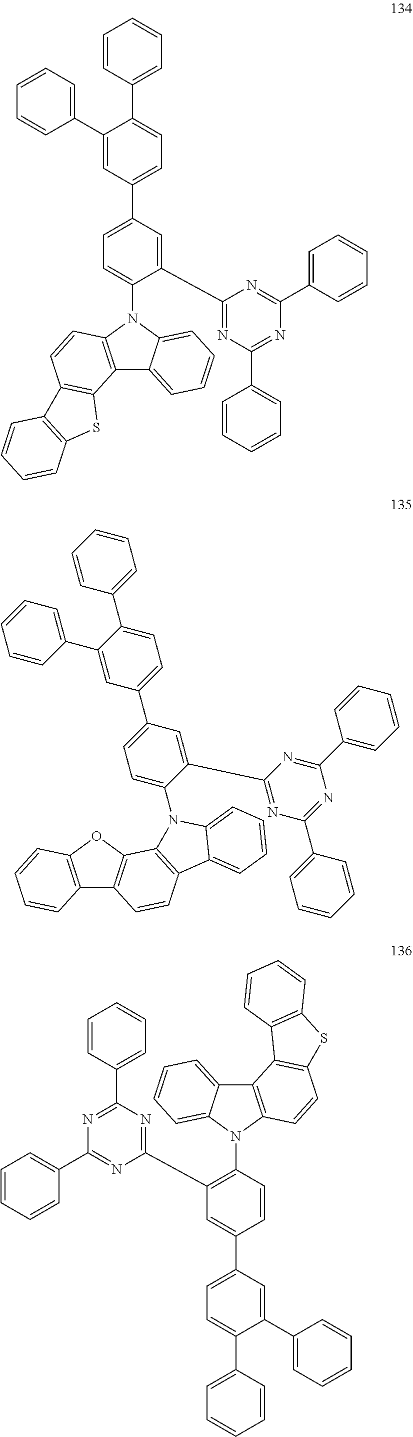 [SMILES: c1ccc(-c2nc(-c3ccccc3)nc(-c3cc(-c4ccc(-c5ccccc5)c(-c5ccccc5)c4)ccc3-n3c4ccccc4c4c5sc6ccccc6c5ccc43)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5c6ccccc6oc5c43)n2)cc1 ; c1ccc(-c2nc(-c3ccccc3)nc(-c3cc(-c4ccc(-c5ccccc5)c(-c5ccccc5)c4)ccc3-n3c4ccccc4c4c5c(ccc43)sc3ccccc35)n2)cc1]

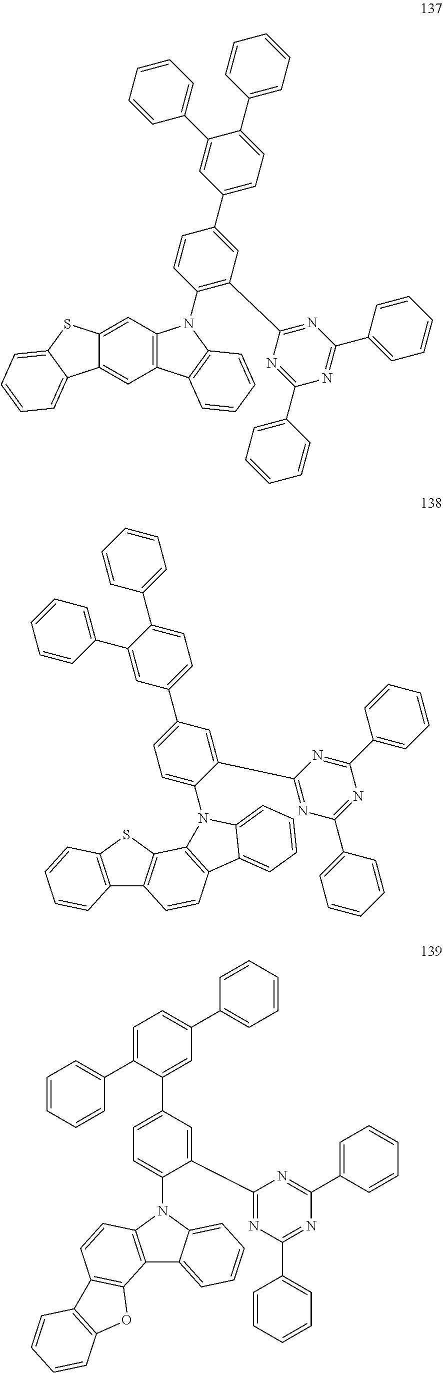 [SMILES: c1ccc(-c2nc(-c3ccccc3)nc(-c3cc(-c4ccc(-c5ccccc5)c(-c5ccccc5)c4)ccc3-n3c4ccccc4c4cc5c(cc43)sc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5c6ccccc6sc5c43)n2)cc1 ; c1ccc(-c2ccc(-c3ccccc3)c(-c3ccc(-n4c5ccccc5c5c6oc7ccccc7c6ccc54)c(-c4nc(-c5ccccc5)nc(-c5ccccc5)n4)c3)c2)cc1]

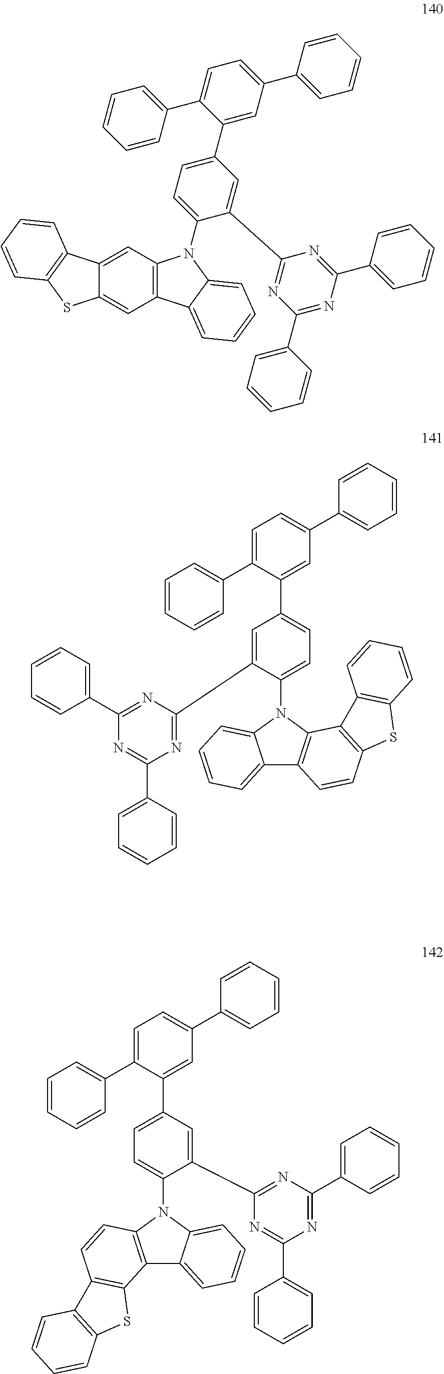 [SMILES: c1ccc(-c2ccc(-c3ccccc3)c(-c3ccc(-n4c5ccccc5c5cc6sc7ccccc7c6cc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2ccc(-c3ccccc3)c(-c3ccc(-n4c5ccccc5c5c6sc7ccccc7c6ccc54)c(-c4nc(-c5ccccc5)nc(-c5ccccc5)n4)c3)c2)cc1]

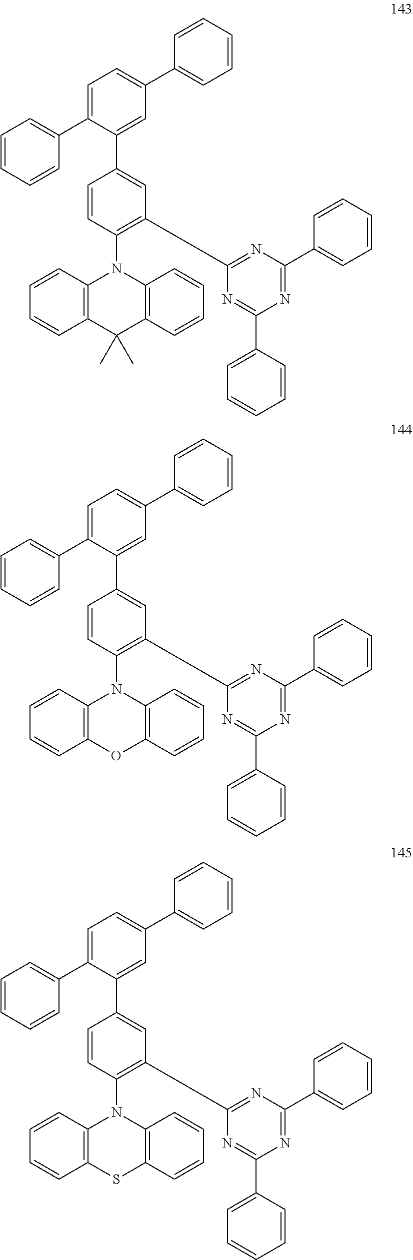 [SMILES: CC1(C)c2ccccc2N(c2ccc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3ccc(N4c5ccccc5Oc5ccccc54)c(-c4nc(-c5ccccc5)nc(-c5ccccc5)n4)c3)c2)cc1 ; c1ccc(-c2ccc(-c3ccccc3)c(-c3ccc(N4c5ccccc5Sc5ccccc54)c(-c4nc(-c5ccccc5)nc(-c5ccccc5)n4)c3)c2)cc1]

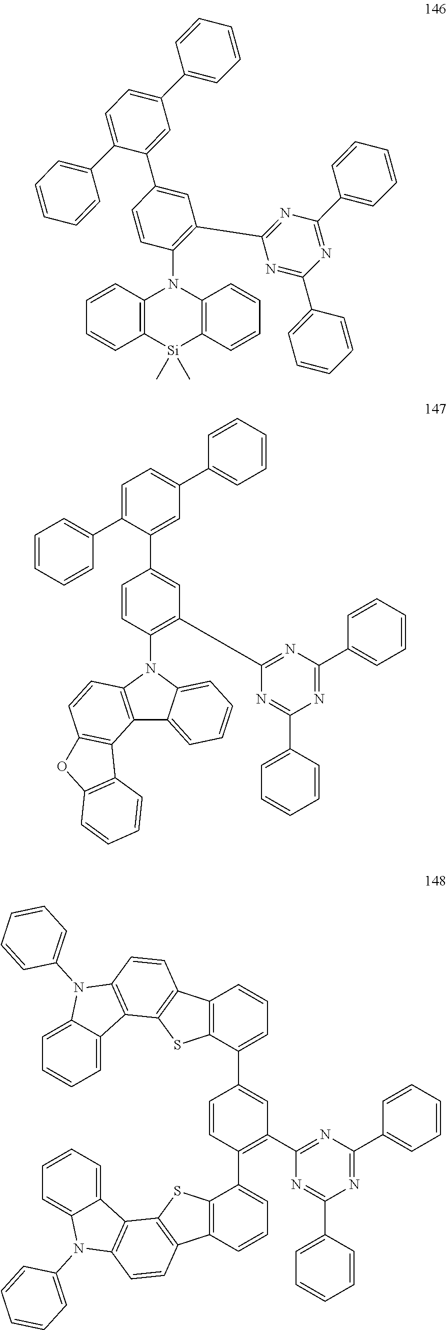 [SMILES: C[Si]1(C)c2ccccc2N(c2ccc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2nc(-c3ccccc3)nc(-c3cc(-c4cccc5c4sc4c5ccc5c4c4ccccc4n5-c4ccccc4)ccc3-c3cccc4c3sc3c4ccc4c3c3ccccc3n4-c3ccccc3)n2)cc1]

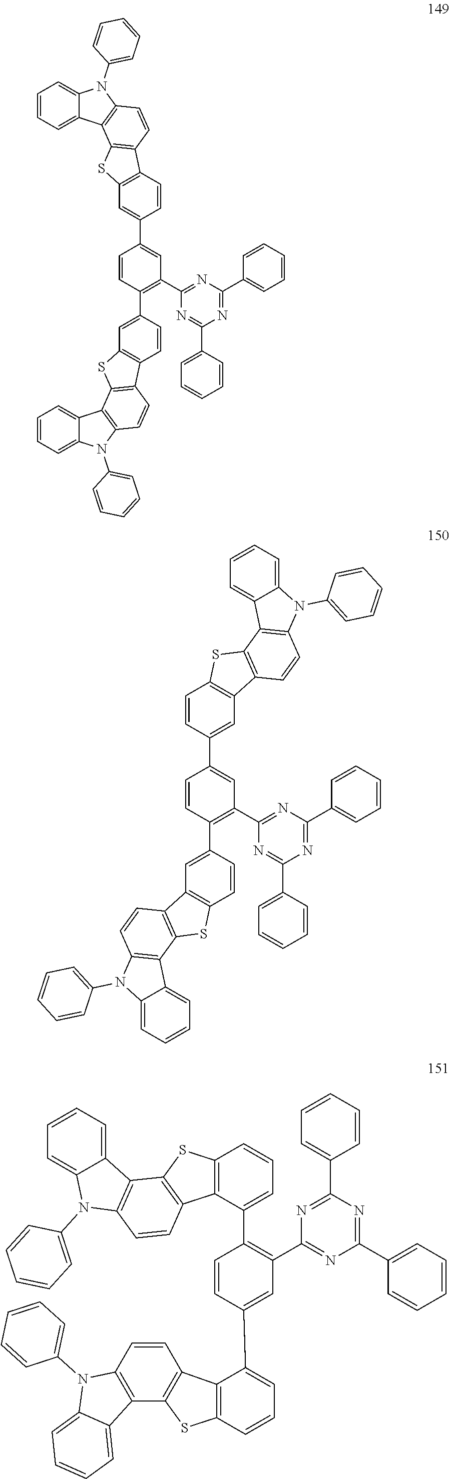 [SMILES: c1ccc(-c2nc(-c3ccccc3)nc(-c3cc(-c4ccc5c(c4)sc4c5ccc5c4c4ccccc4n5-c4ccccc4)ccc3-c3ccc4c(c3)sc3c4ccc4c3c3ccccc3n4-c3ccccc3)n2)cc1 ; c1ccc(-c2nc(-c3ccccc3)nc(-c3cc(-c4ccc5sc6c(ccc7c6c6ccccc6n7-c6ccccc6)c5c4)ccc3-c3ccc4sc5c(ccc6c5c5ccccc5n6-c5ccccc5)c4c3)n2)cc1 ; c1ccc(-c2nc(-c3ccccc3)nc(-c3cc(-c4cccc5sc6c(ccc7c6c6ccccc6n7-c6ccccc6)c45)ccc3-c3cccc4sc5c(ccc6c5c5ccccc5n6-c5ccccc5)c34)n2)cc1]

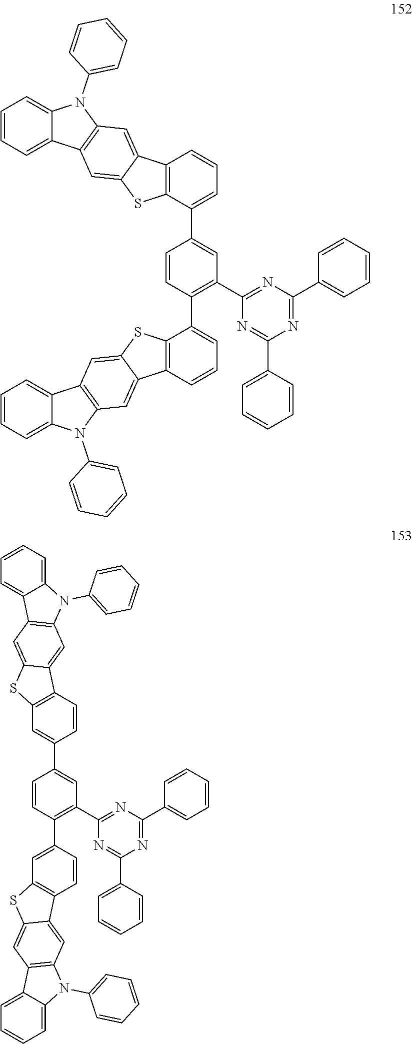 [SMILES: c1ccc(-c2nc(-c3ccccc3)nc(-c3cc(-c4cccc5c4sc4cc6c7ccccc7n(-c7ccccc7)c6cc45)ccc3-c3cccc4c3sc3cc5c6ccccc6n(-c6ccccc6)c5cc34)n2)cc1 ; c1ccc(-c2nc(-c3ccccc3)nc(-c3cc(-c4ccc5c(c4)sc4cc6c7ccccc7n(-c7ccccc7)c6cc45)ccc3-c3ccc4c(c3)sc3cc5c6ccccc6n(-c6ccccc6)c5cc34)n2)cc1]

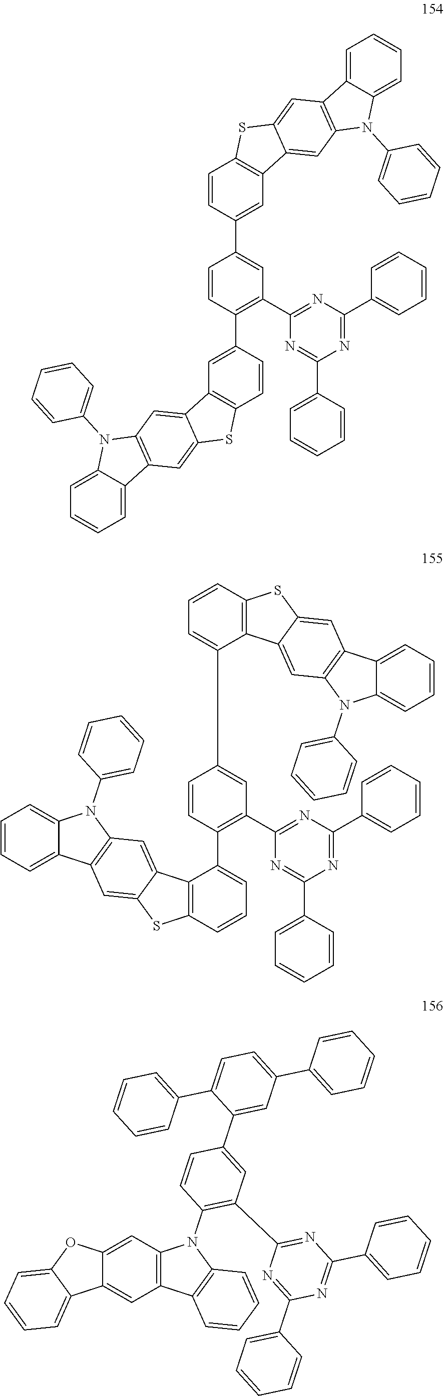 [SMILES: c1ccc(-c2nc(-c3ccccc3)nc(-c3cc(-c4ccc5sc6cc7c8ccccc8n(-c8ccccc8)c7cc6c5c4)ccc3-c3ccc4sc5cc6c7ccccc7n(-c7ccccc7)c6cc5c4c3)n2)cc1 ; c1ccc(-c2nc(-c3ccccc3)nc(-c3cc(-c4cccc5sc6cc7c8ccccc8n(-c8ccccc8)c7cc6c45)ccc3-c3cccc4sc5cc6c7ccccc7n(-c7ccccc7)c6cc5c34)n2)cc1 ; c1ccc(-c2ccc(-c3ccccc3)c(-c3ccc(-n4c5ccccc5c5cc6c(cc54)oc4ccccc46)c(-c4nc(-c5ccccc5)nc(-c5ccccc5)n4)c3)c2)cc1]

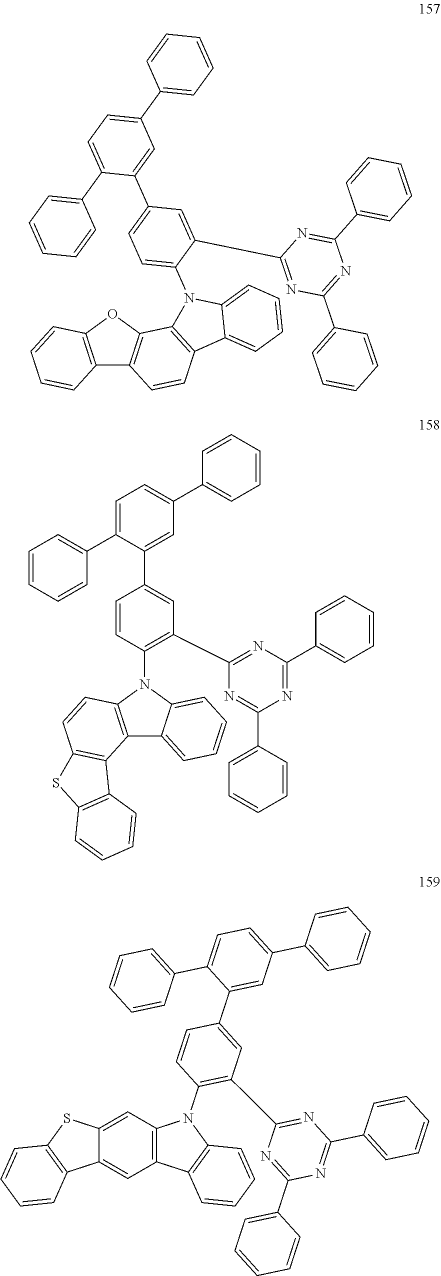 [SMILES: c1ccc(-c2ccc(-c3ccccc3)c(-c3ccc(-n4c5ccccc5c5ccc6c7ccccc7oc6c54)c(-c4nc(-c5ccccc5)nc(-c5ccccc5)n4)c3)c2)cc1 ; c1ccc(-c2ccc(-c3ccccc3)c(-c3ccc(-n4c5ccccc5c5c6c(ccc54)sc4ccccc46)c(-c4nc(-c5ccccc5)nc(-c5ccccc5)n4)c3)c2)cc1 ; c1ccc(-c2ccc(-c3ccccc3)c(-c3ccc(-n4c5ccccc5c5cc6c(cc54)sc4ccccc46)c(-c4nc(-c5ccccc5)nc(-c5ccccc5)n4)c3)c2)cc1]

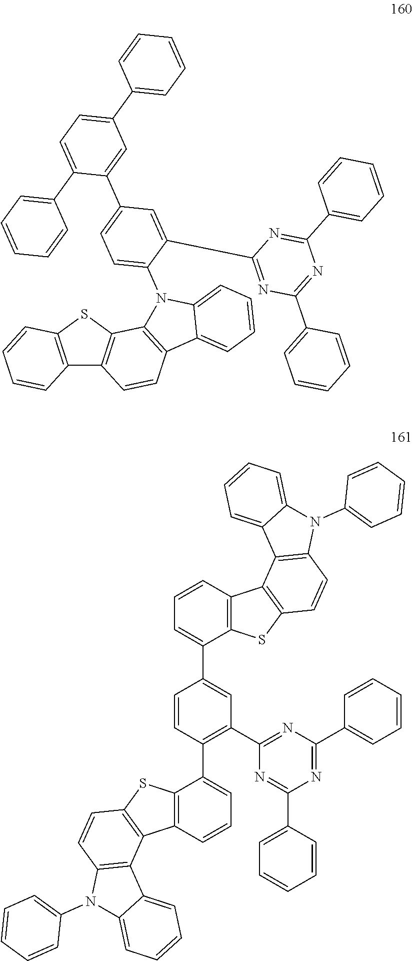 [SMILES: c1ccc(-c2ccc(-c3ccccc3)c(-c3ccc(-n4c5ccccc5c5ccc6c7ccccc7sc6c54)c(-c4nc(-c5ccccc5)nc(-c5ccccc5)n4)c3)c2)cc1 ; c1ccc(-c2nc(-c3ccccc3)nc(-c3cc(-c4cccc5c4sc4ccc6c(c7ccccc7n6-c6ccccc6)c45)ccc3-c3cccc4c3sc3ccc5c(c6ccccc6n5-c5ccccc5)c34)n2)cc1]

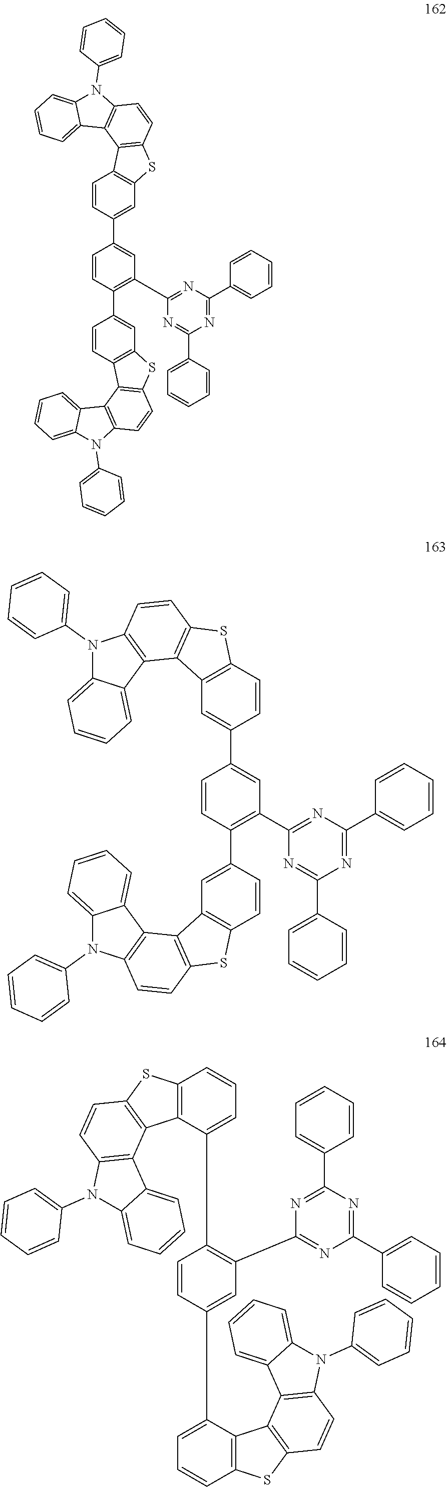 [SMILES: c1ccc(-c2nc(-c3ccccc3)nc(-c3cc(-c4ccc5c(c4)sc4ccc6c(c7ccccc7n6-c6ccccc6)c45)ccc3-c3ccc4c(c3)sc3ccc5c(c6ccccc6n5-c5ccccc5)c34)n2)cc1 ; c1ccc(-c2nc(-c3ccccc3)nc(-c3cc(-c4ccc5sc6ccc7c(c8ccccc8n7-c7ccccc7)c6c5c4)ccc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)ccc3-c3cccc4sc5ccc6c(c7ccccc7n6-c6ccccc6)c5c34)n2)cc1]

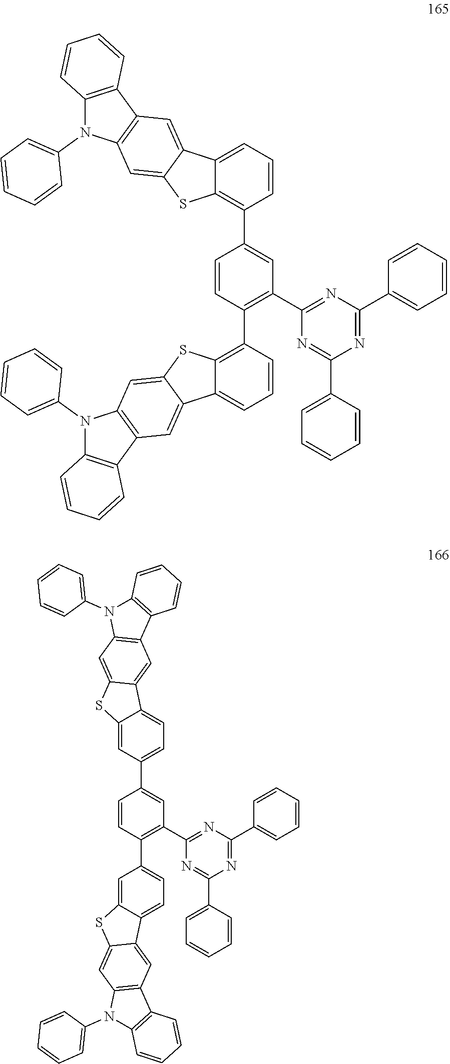 [SMILES: c1ccc(-c2nc(-c3ccccc3)nc(-c3cc(-c4cccc5c4sc4cc6c(cc45)c4ccccc4n6-c4ccccc4)ccc3-c3cccc4c3sc3cc5c(cc34)c3ccccc3n5-c3ccccc3)n2)cc1 ; c1ccc(-c2nc(-c3ccccc3)nc(-c3cc(-c4ccc5c(c4)sc4cc6c(cc45)c4ccccc4n6-c4ccccc4)ccc3-c3ccc4c(c3)sc3cc5c(cc34)c3ccccc3n5-c3ccccc3)n2)cc1]

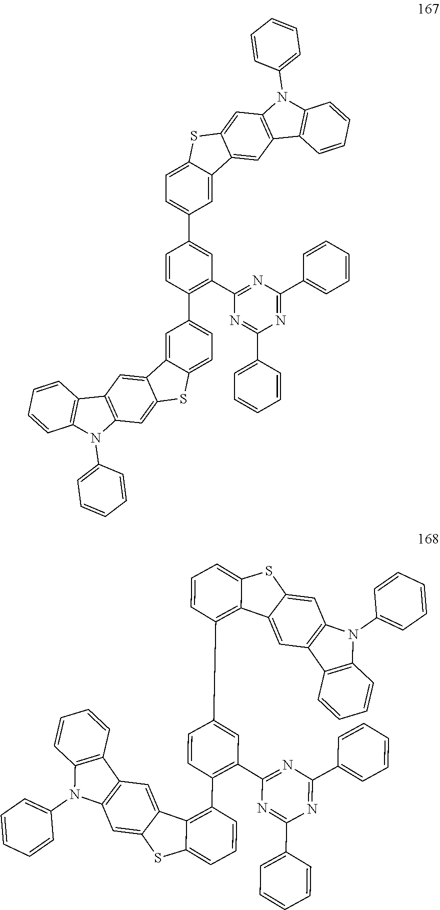 [SMILES: c1ccc(-c2nc(-c3ccccc3)nc(-c3cc(-c4ccc5sc6cc7c(cc6c5c4)c4ccccc4n7-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4cccc5sc6cc7c(cc6c45)c4ccccc4n7-c4ccccc4)ccc3-c3cccc4sc5cc6c(cc5c34)c3ccccc3n6-c3ccccc3)n2)cc1]

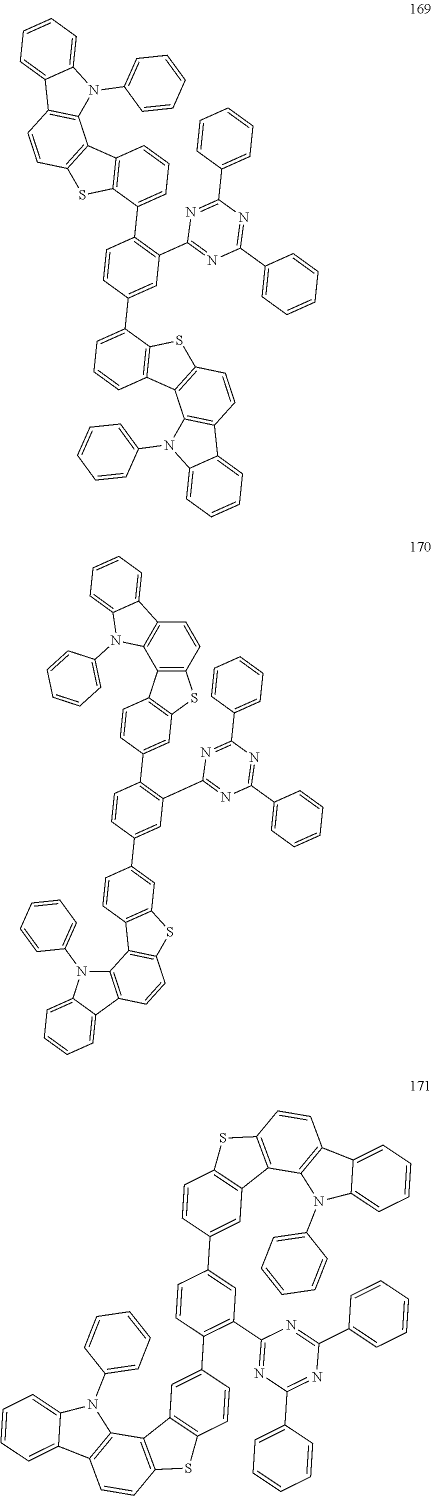 [SMILES: c1ccc(-c2nc(-c3ccccc3)nc(-c3cc(-c4cccc5c4sc4ccc6c7ccccc7n(-c7ccccc7)c6c45)ccc3-c3cccc4c3sc3ccc5c6ccccc6n(-c6ccccc6)c5c34)n2)cc1 ; c1ccc(-c2nc(-c3ccccc3)nc(-c3cc(-c4ccc5c(c4)sc4ccc6c7ccccc7n(-c7ccccc7)c6c45)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccc5sc6ccc7c8ccccc8n(-c8ccccc8)c7c6c5c4)ccc3-c3ccc4sc5ccc6c7ccccc7n(-c7ccccc7)c6c5c4c3)n2)cc1]

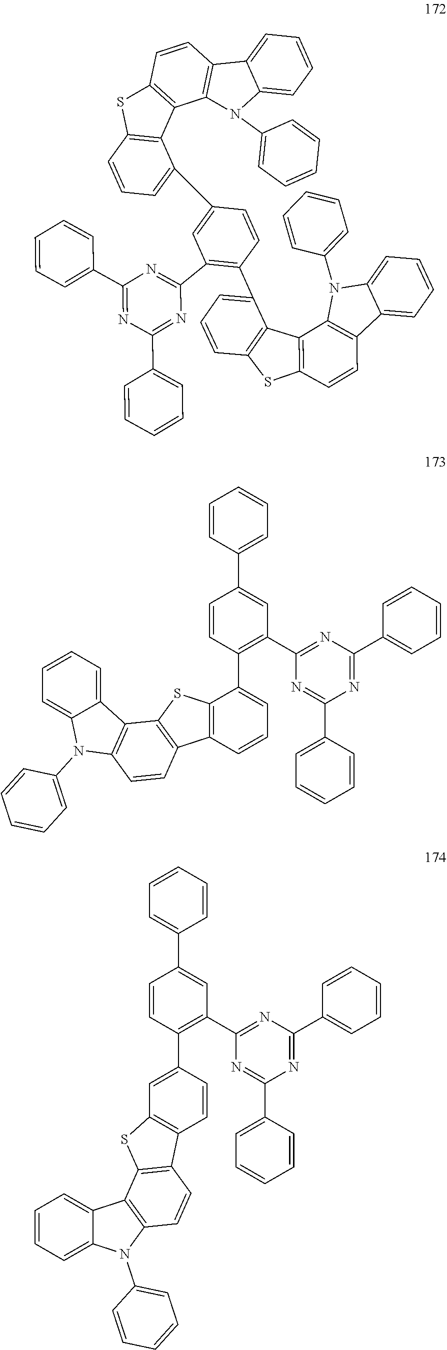 [SMILES: c1ccc(-c2nc(-c3ccccc3)nc(-c3cc(-c4cccc5sc6ccc7c8ccccc8n(-c8ccccc8)c7c6c45)ccc3-c3cccc4sc5ccc6c7ccccc7n(-c7ccccc7)c6c5c34)n2)cc1 ; c1ccc(-c2ccc(-c3cccc4c3sc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)sc3c4ccc4c3c3ccccc3n4-c3ccccc3)c(-c3nc(-c4ccccc4)nc(-c4ccccc4)n3)c2)cc1]

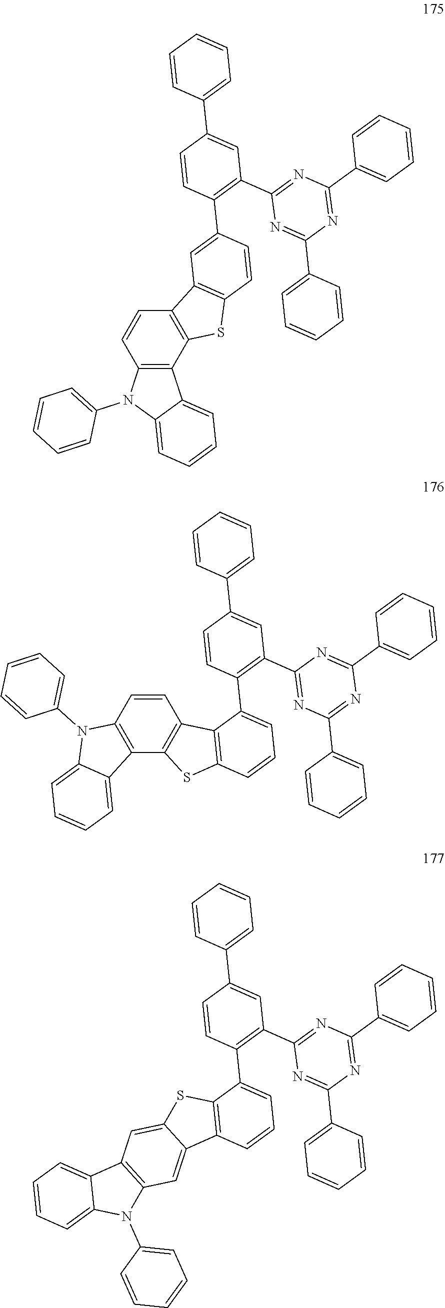 [SMILES: c1ccc(-c2ccc(-c3ccc4sc5c(ccc6c5c5ccccc5n6-c5ccccc5)c4c3)c(-c3nc(-c4ccccc4)nc(-c4ccccc4)n3)c2)cc1 ; c1ccc(-c2ccc(-c3cccc4sc5c(ccc6c5c5ccccc5n6-c5ccccc5)c34)c(-c3nc(-c4ccccc4)nc(-c4ccccc4)n3)c2)cc1 ; c1ccc(-c2ccc(-c3cccc4c3sc3cc5c6ccccc6n(-c6ccccc6)c5cc34)c(-c3nc(-c4ccccc4)nc(-c4ccccc4)n3)c2)cc1]

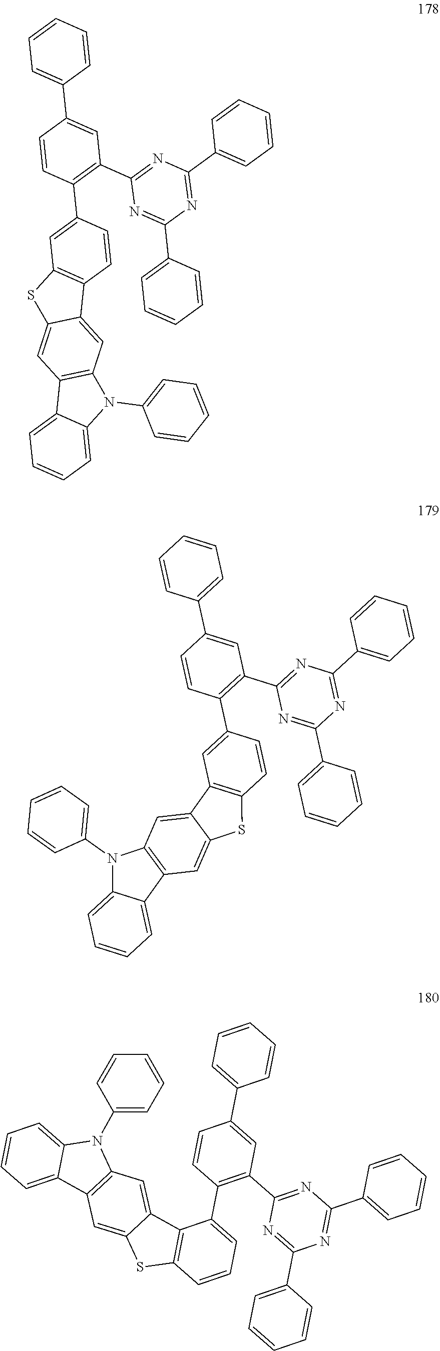 [SMILES: c1ccc(-c2ccc(-c3ccc4c(c3)sc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4sc5cc6c7ccccc7n(-c7ccccc7)c6cc5c34)c(-c3nc(-c4ccccc4)nc(-c4ccccc4)n3)c2)cc1]

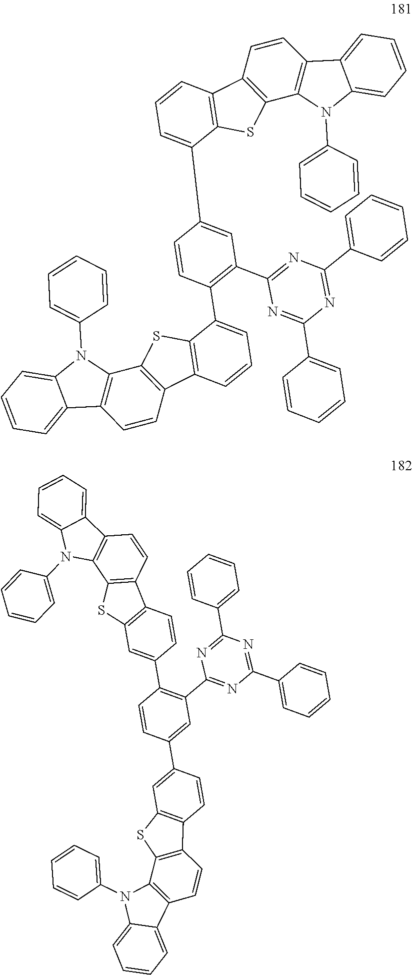 [SMILES: c1ccc(-c2nc(-c3ccccc3)nc(-c3cc(-c4cccc5c4sc4c5ccc5c6ccccc6n(-c6ccccc6)c54)ccc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)ccc3-c3ccc4c(c3)sc3c4ccc4c5ccccc5n(-c5ccccc5)c43)n2)cc1]

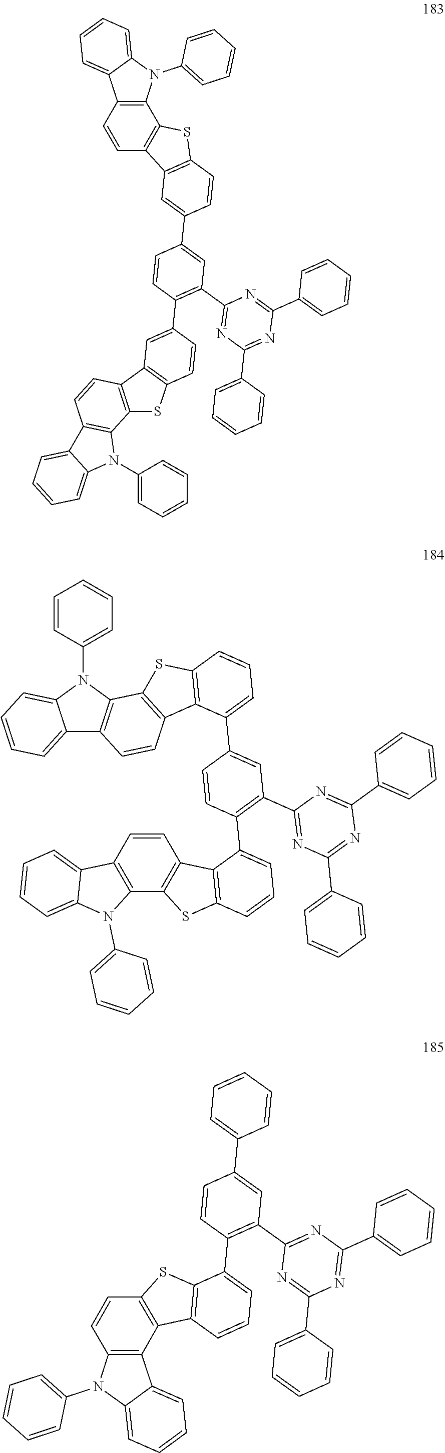 [SMILES: c1ccc(-c2nc(-c3ccccc3)nc(-c3cc(-c4ccc5sc6c(ccc7c8ccccc8n(-c8ccccc8)c76)c5c4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4cccc5sc6c(ccc7c8ccccc8n(-c8ccccc8)c76)c45)ccc3-c3cccc4sc5c(ccc6c7ccccc7n(-c7ccccc7)c65)c34)n2)cc1 ; c1ccc(-c2ccc(-c3cccc4c3sc3ccc5c(c6ccccc6n5-c5ccccc5)c34)c(-c3nc(-c4ccccc4)nc(-c4ccccc4)n3)c2)cc1]

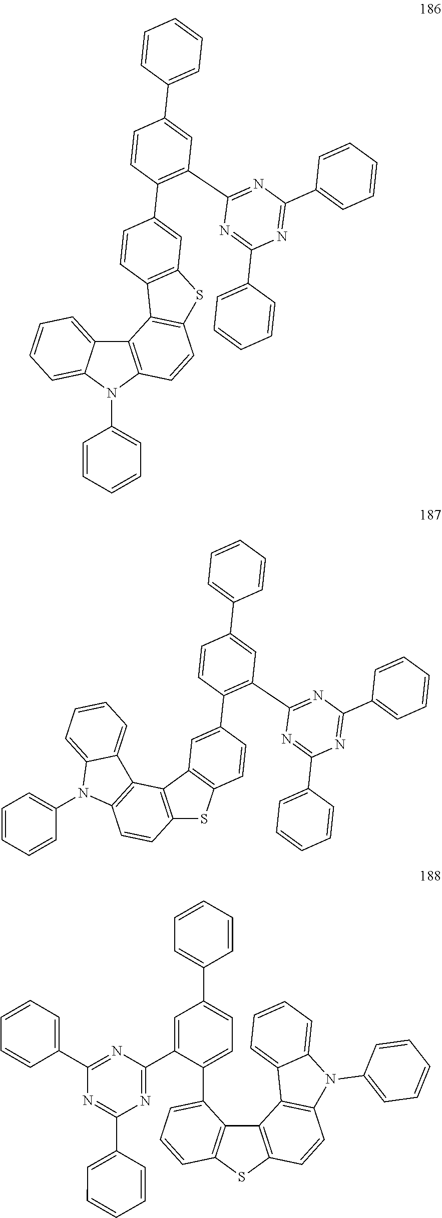 [SMILES: c1ccc(-c2ccc(-c3ccc4c(c3)sc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4sc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4sc5ccc6c(c7ccccc7n6-c6ccccc6)c5c34)c(-c3nc(-c4ccccc4)nc(-c4ccccc4)n3)c2)cc1]

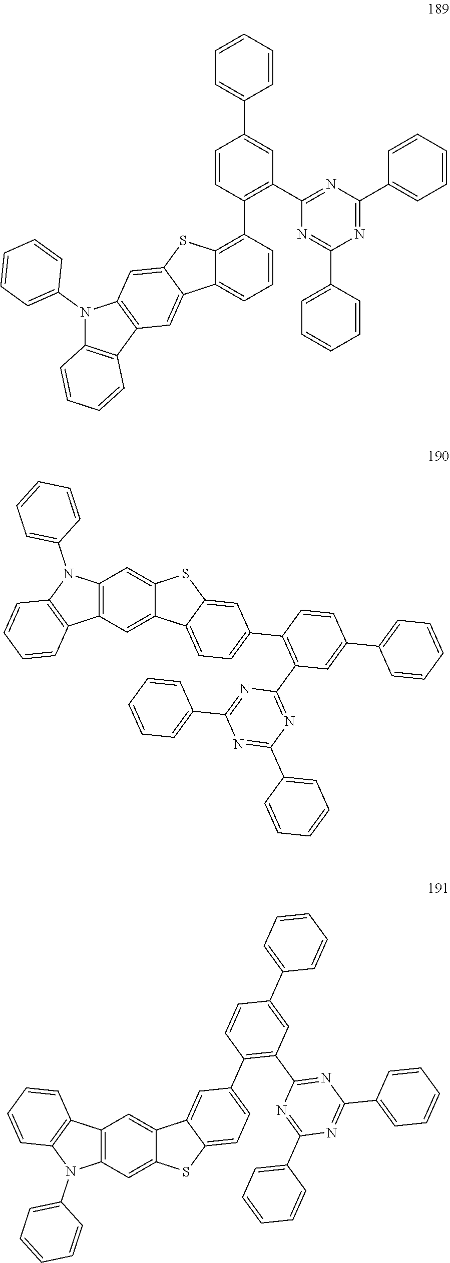 [SMILES: c1ccc(-c2ccc(-c3cccc4c3sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4sc5cc6c(cc5c4c3)c3ccccc3n6-c3ccccc3)c(-c3nc(-c4ccccc4)nc(-c4ccccc4)n3)c2)cc1]

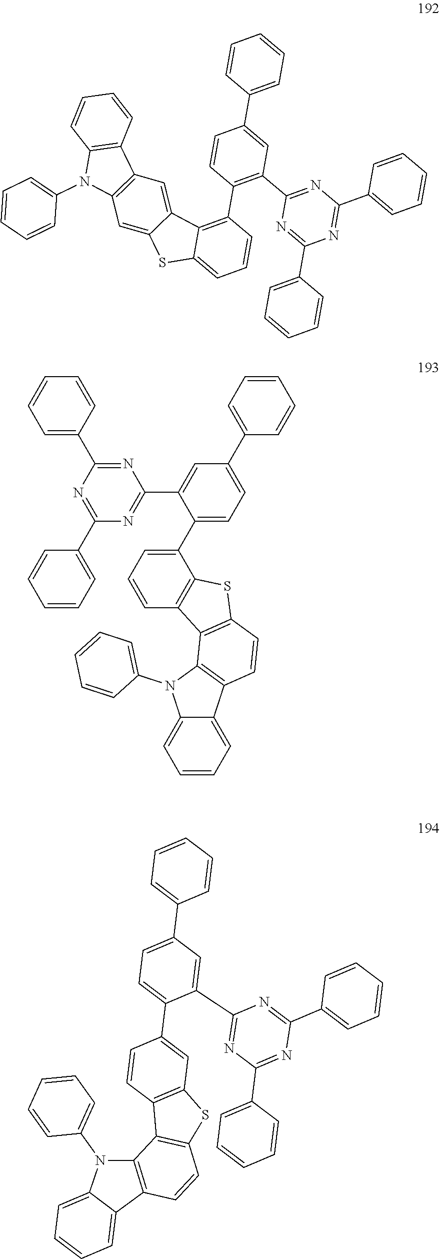

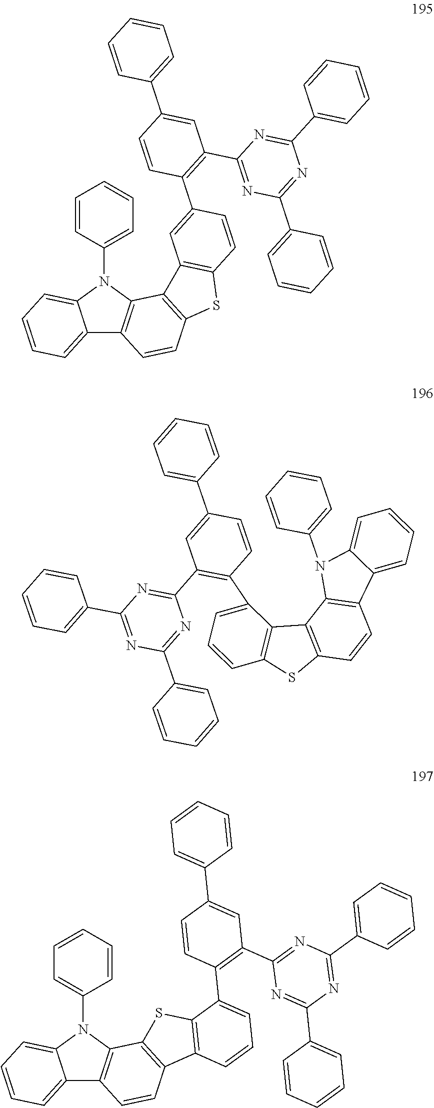 [SMILES: c1ccc(-c2ccc(-c3ccc4sc5ccc6c7ccccc7n(-c7ccccc7)c6c5c4c3)c(-c3nc(-c4ccccc4)nc(-c4ccccc4)n3)c2)cc1 ; c1ccc(-c2ccc(-c3cccc4sc5ccc6c7ccccc7n(-c7ccccc7)c6c5c34)c(-c3nc(-c4ccccc4)nc(-c4ccccc4)n3)c2)cc1 ; c1ccc(-c2ccc(-c3cccc4c3sc3c4ccc4c5ccccc5n(-c5ccccc5)c43)c(-c3nc(-c4ccccc4)nc(-c4ccccc4)n3)c2)cc1]

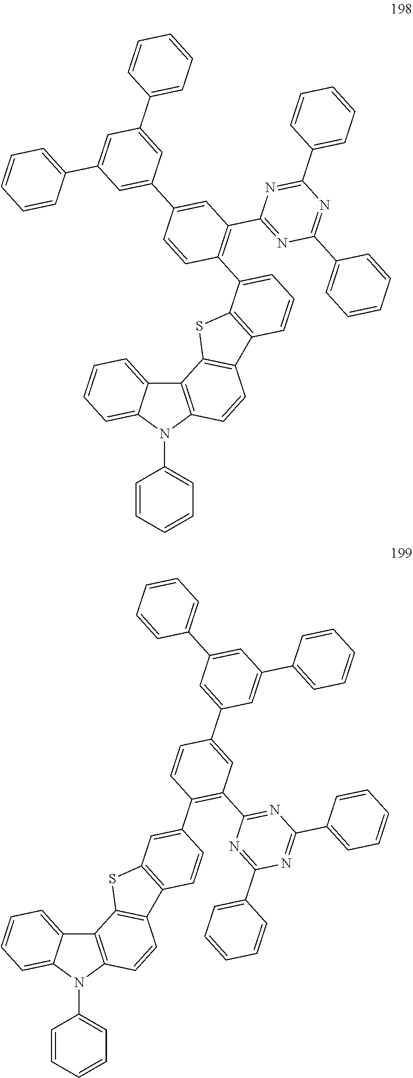 [SMILES: c1ccc(-c2cc(-c3ccccc3)cc(-c3ccc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1]

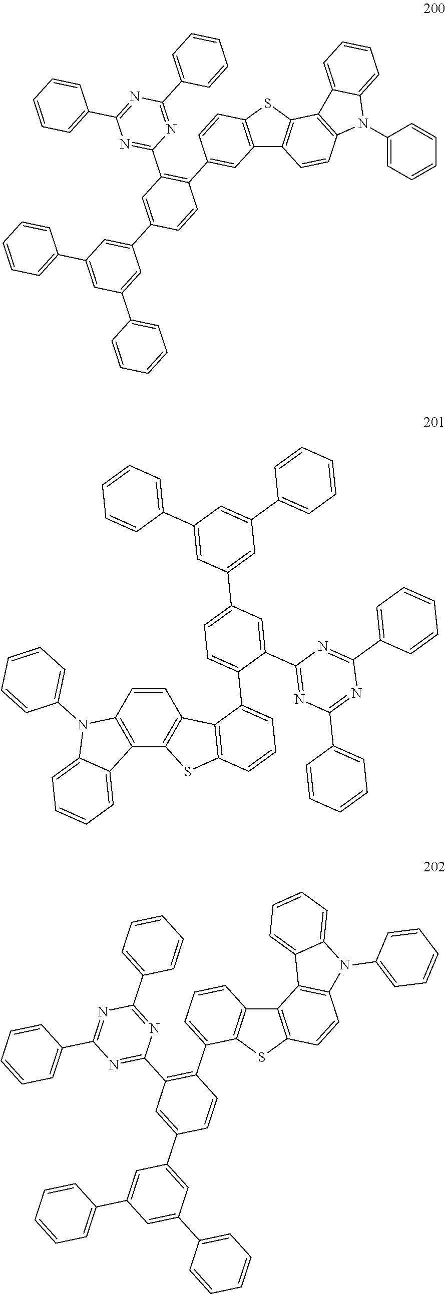 [SMILES: c1ccc(-c2cc(-c3ccccc3)cc(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

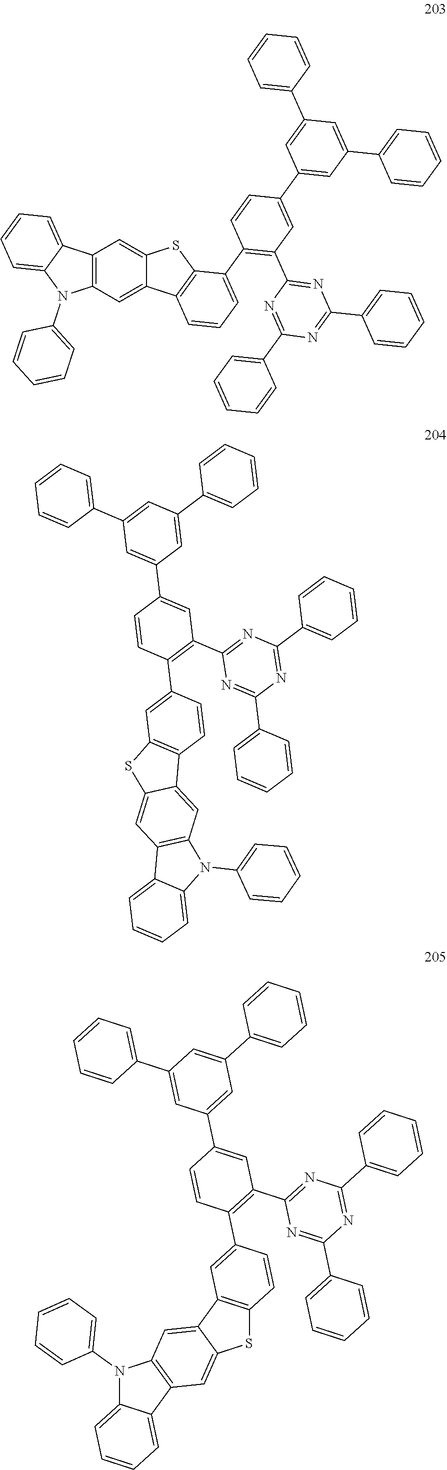

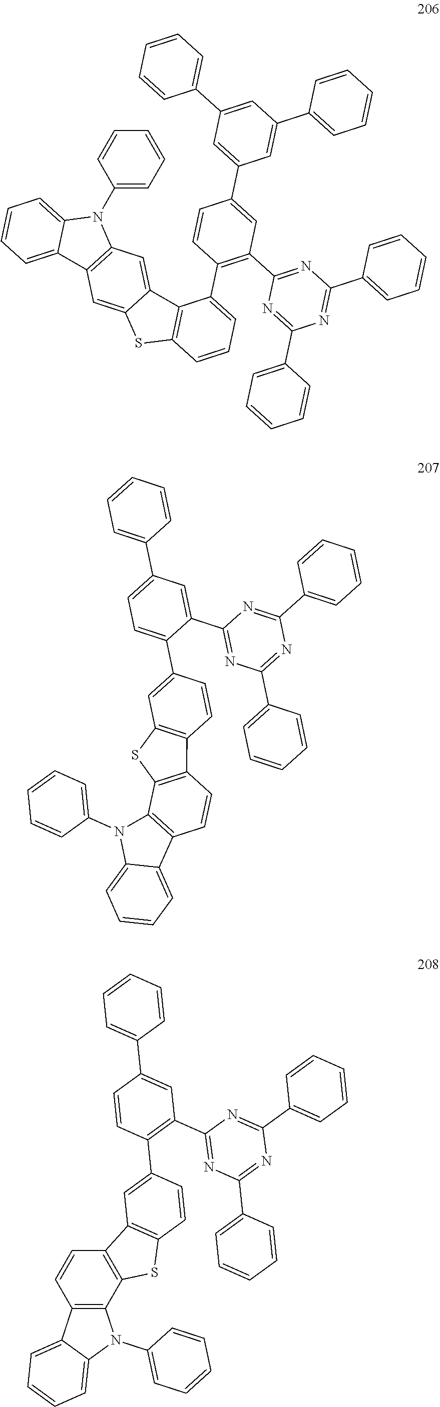 [SMILES: c1ccc(-c2cc(-c3ccccc3)cc(-c3ccc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2ccc(-c3ccc4c(c3)sc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4sc5c(ccc6c7ccccc7n(-c7ccccc7)c65)c4c3)c(-c3nc(-c4ccccc4)nc(-c4ccccc4)n3)c2)cc1]

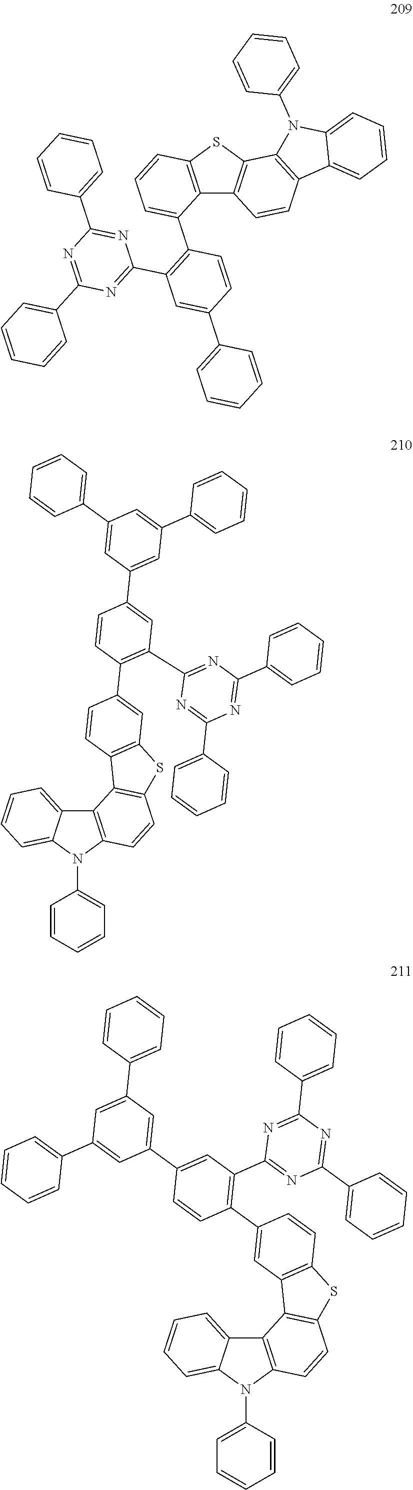 [SMILES: c1ccc(-c2ccc(-c3cccc4sc5c(ccc6c7ccccc7n(-c7ccccc7)c65)c34)c(-c3nc(-c4ccccc4)nc(-c4ccccc4)n3)c2)cc1 ; c1ccc(-c2cc(-c3ccccc3)cc(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1]

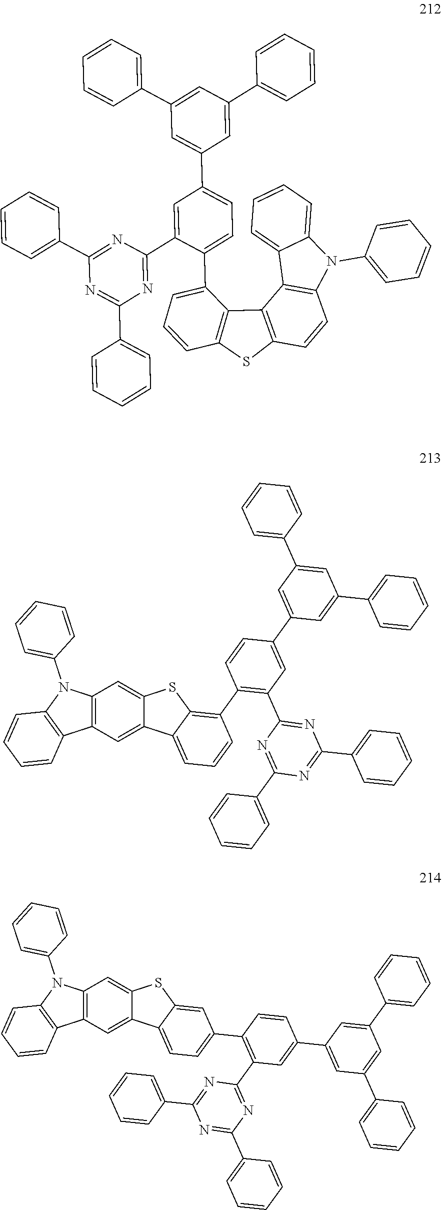 [SMILES: c1ccc(-c2cc(-c3ccccc3)cc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1]

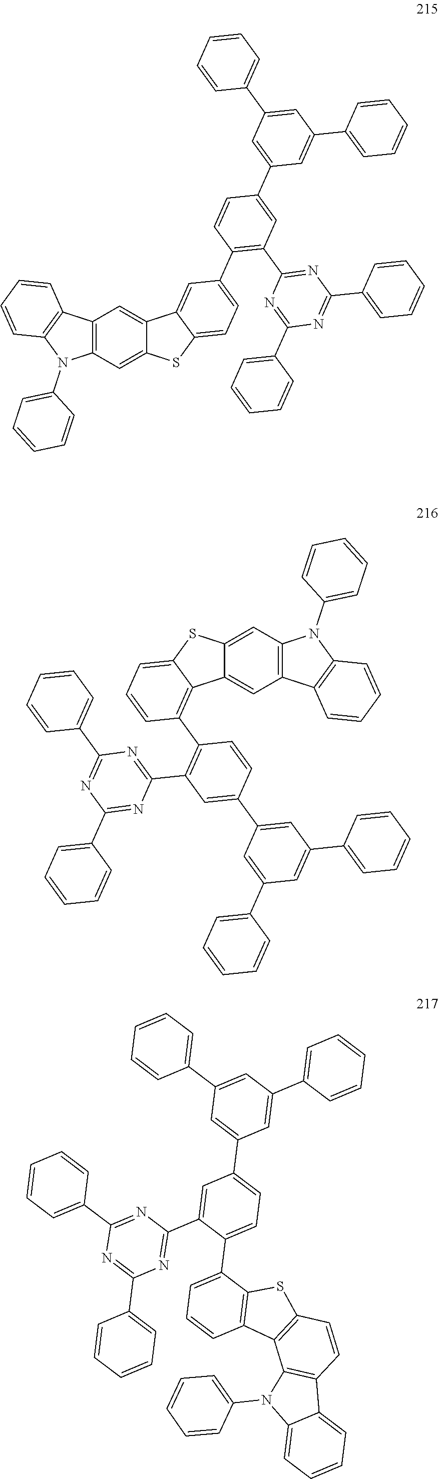 [SMILES: c1ccc(-c2cc(-c3ccccc3)cc(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5c4sc4ccc6c7ccccc7n(-c7ccccc7)c6c45)c(-c4nc(-c5ccccc5)nc(-c5ccccc5)n4)c3)c2)cc1]

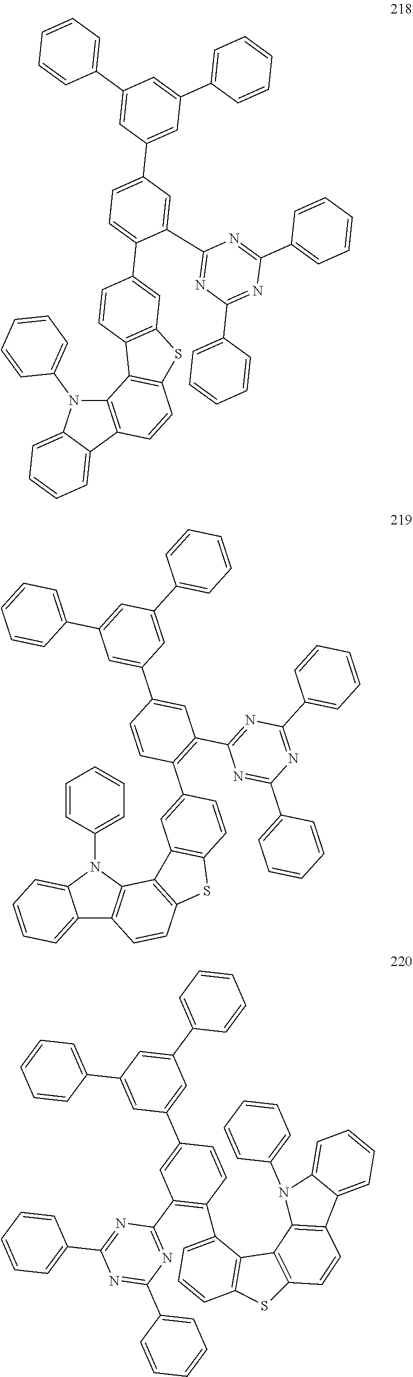 [SMILES: c1ccc(-c2cc(-c3ccccc3)cc(-c3ccc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6ccc7c8ccccc8n(-c8ccccc8)c7c6c45)c(-c4nc(-c5ccccc5)nc(-c5ccccc5)n4)c3)c2)cc1]

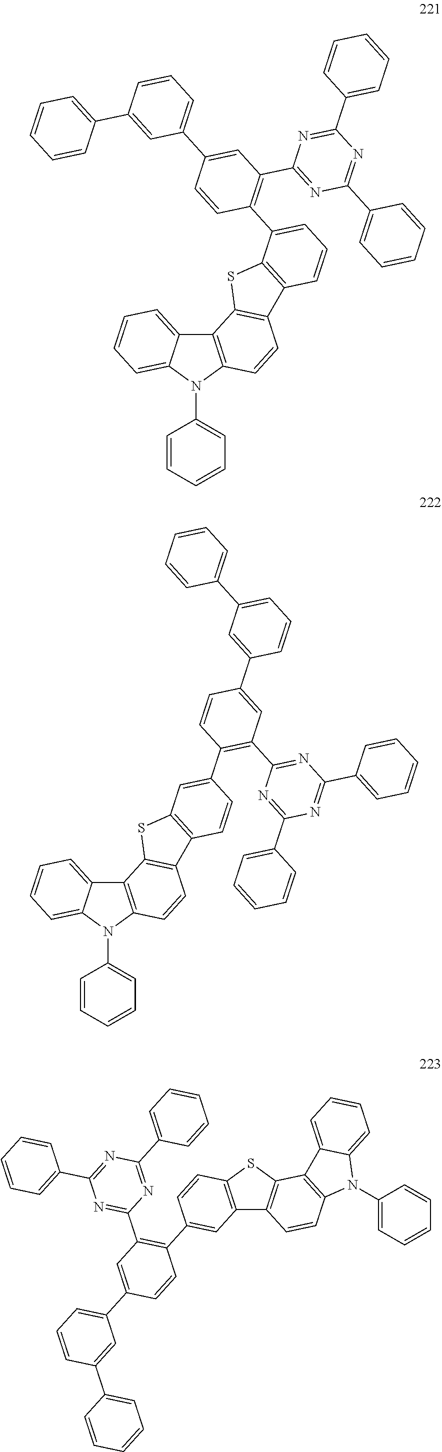

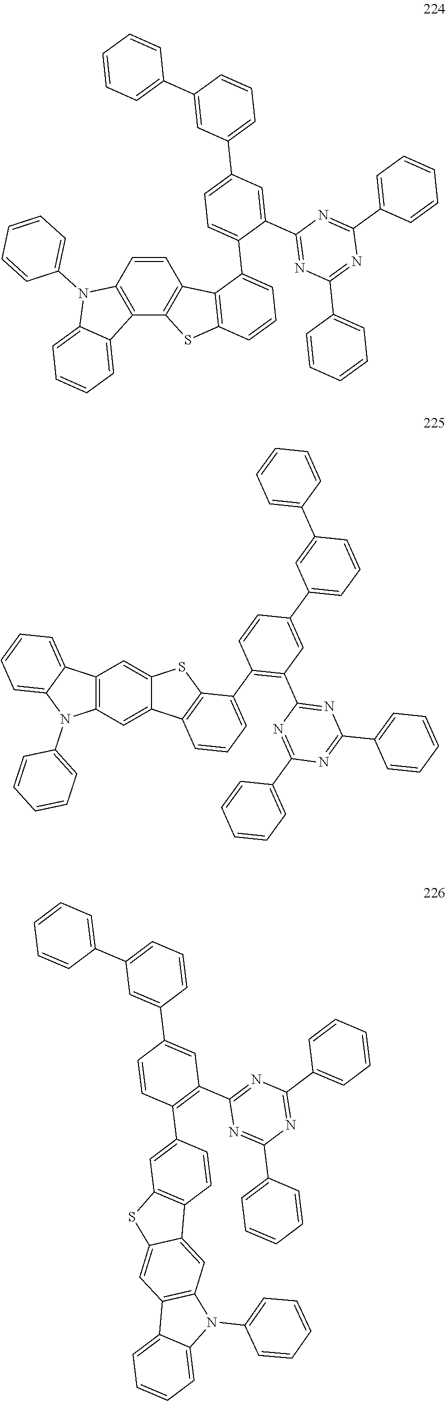 [SMILES: c1ccc(-c2cccc(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3ccc(-c4cccc5c4sc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1]

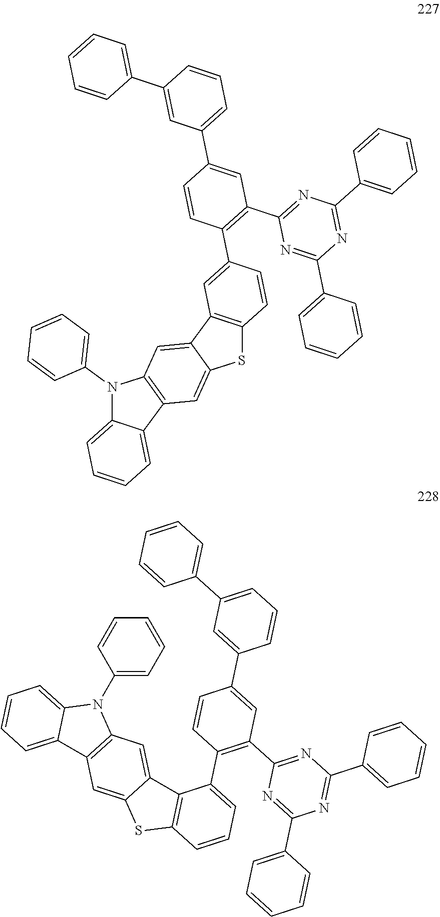 [SMILES: c1ccc(-c2cccc(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3ccc(-c4cccc5sc6cc7c8ccccc8n(-c8ccccc8)c7cc6c45)c(-c4nc(-c5ccccc5)nc(-c5ccccc5)n4)c3)c2)cc1]

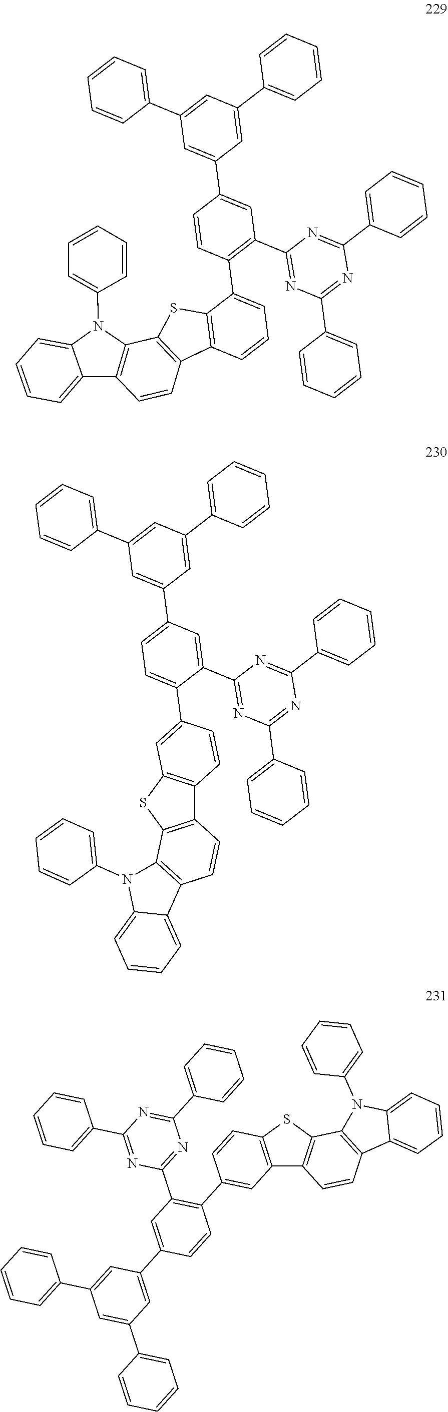 [SMILES: c1ccc(-c2cc(-c3ccccc3)cc(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1]

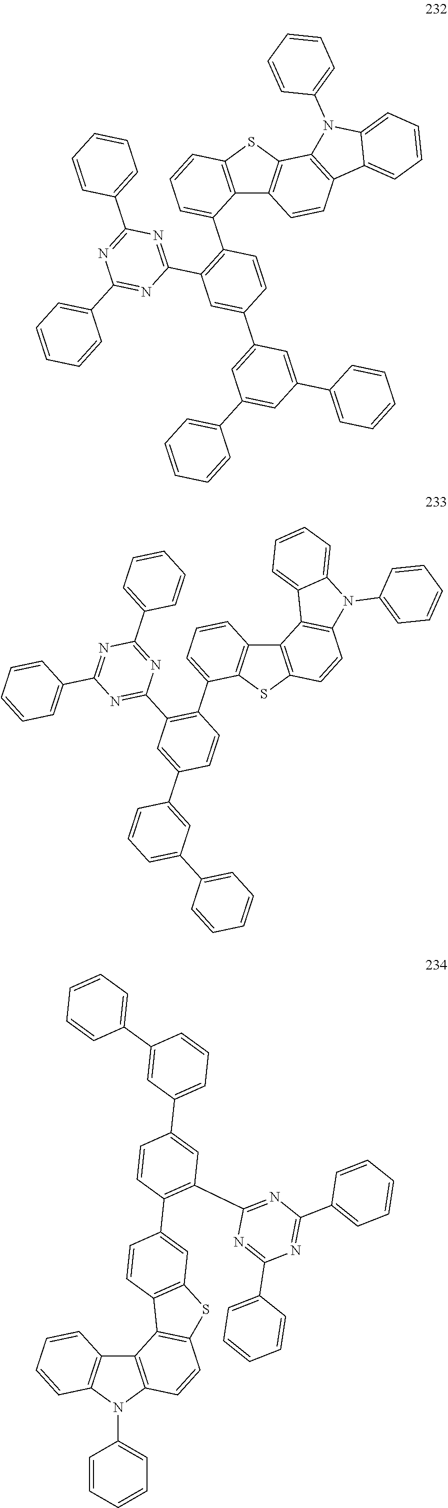 [SMILES: c1ccc(-c2cc(-c3ccccc3)cc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1]

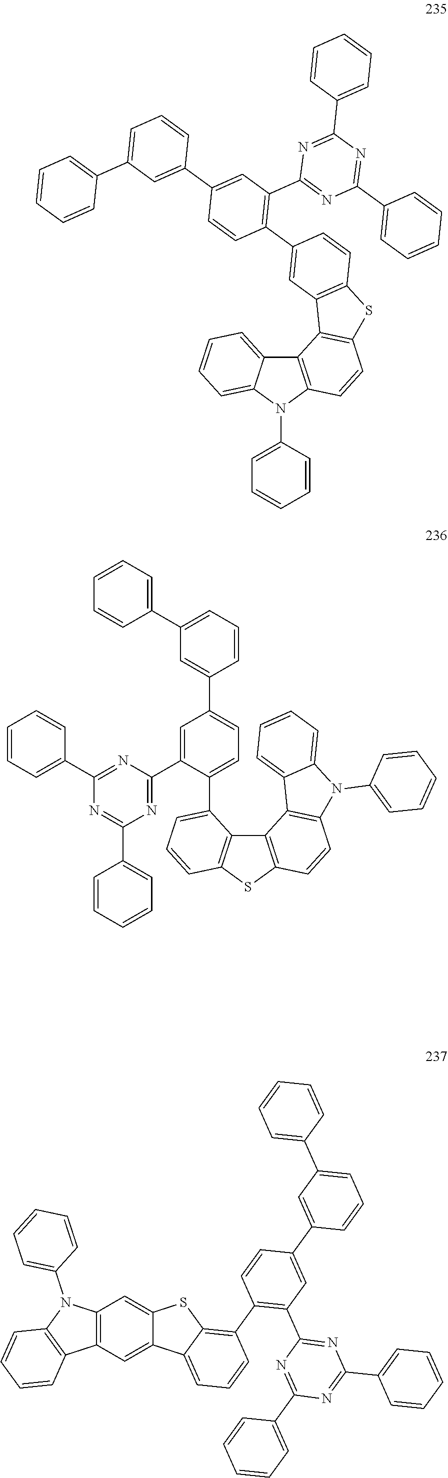 [SMILES: c1ccc(-c2cccc(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4sc4cc6c(cc45)c4ccccc4n6-c4ccccc4)c(-c4nc(-c5ccccc5)nc(-c5ccccc5)n4)c3)c2)cc1]

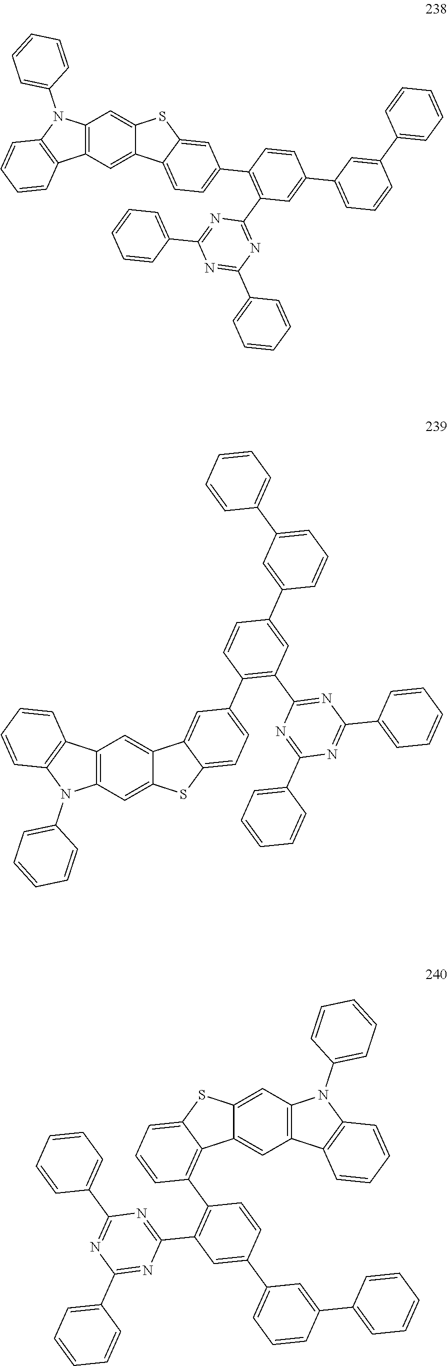 [SMILES: c1ccc(-c2cccc(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5sc6cc7c(cc6c45)c4ccccc4n7-c4ccccc4)c(-c4nc(-c5ccccc5)nc(-c5ccccc5)n4)c3)c2)cc1]

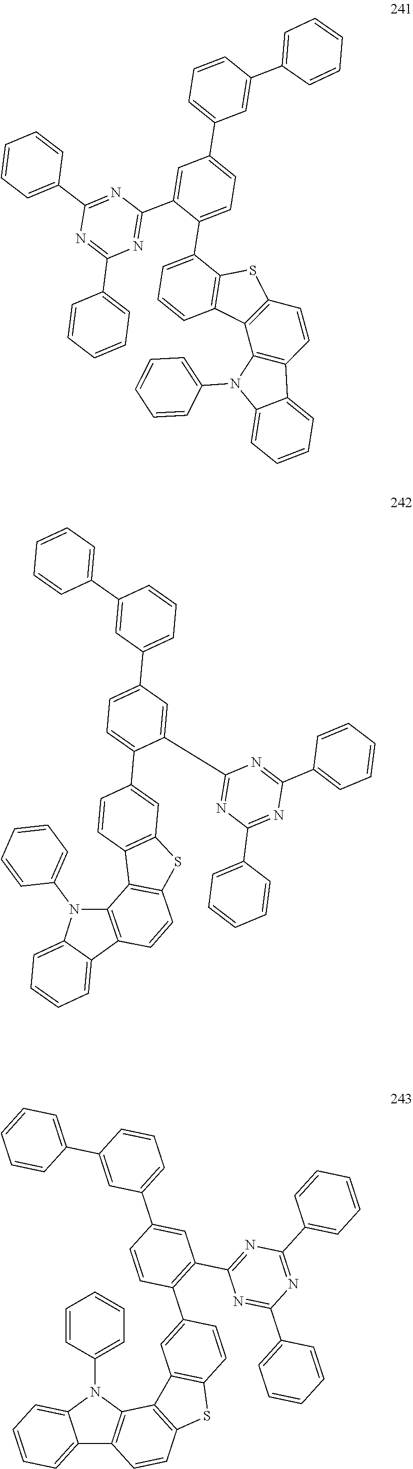 [SMILES: c1ccc(-c2cccc(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1]

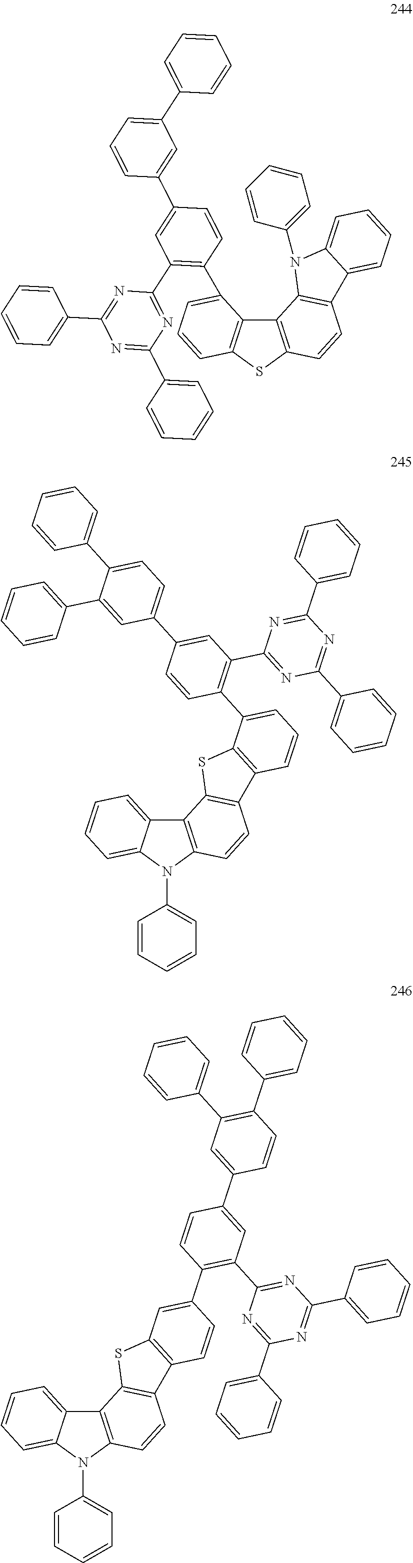 [SMILES: c1ccc(-c2cccc(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)sc3c4ccc4c3c3ccccc3n4-c3ccccc3)n2)cc1]

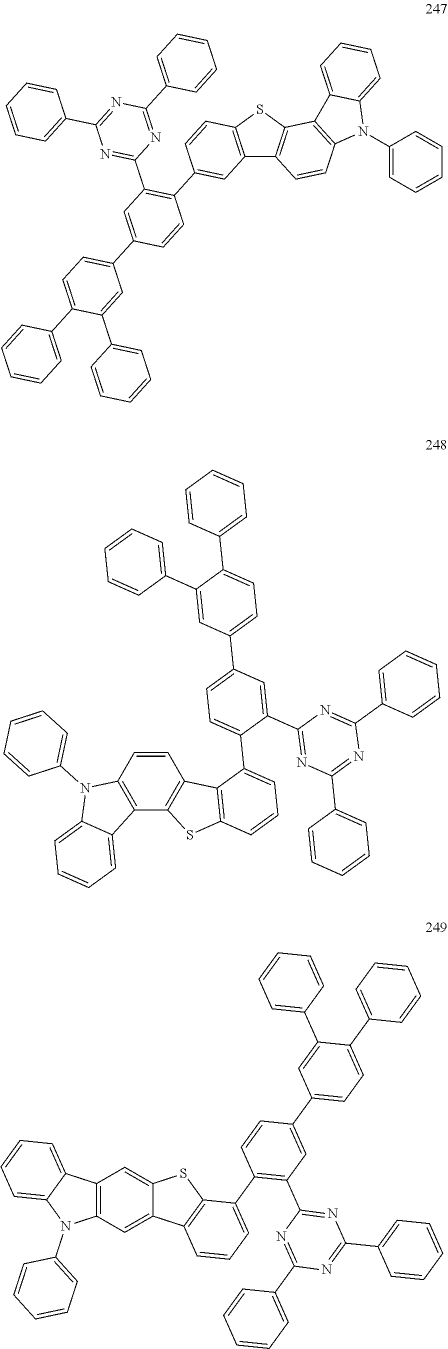 [SMILES: c1ccc(-c2nc(-c3ccccc3)nc(-c3cc(-c4ccc(-c5ccccc5)c(-c5ccccc5)c4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4sc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)ccc3-c3cccc4c3sc3cc5c6ccccc6n(-c6ccccc6)c5cc34)n2)cc1]

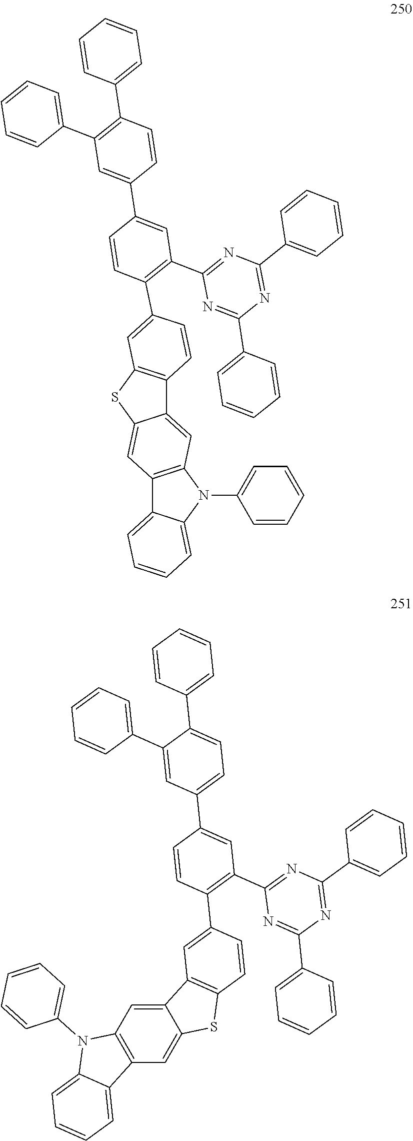 [SMILES: c1ccc(-c2nc(-c3ccccc3)nc(-c3cc(-c4ccc(-c5ccccc5)c(-c5ccccc5)c4)ccc3-c3ccc4c(c3)sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)ccc3-c3ccc4sc5cc6c7ccccc7n(-c7ccccc7)c6cc5c4c3)n2)cc1]

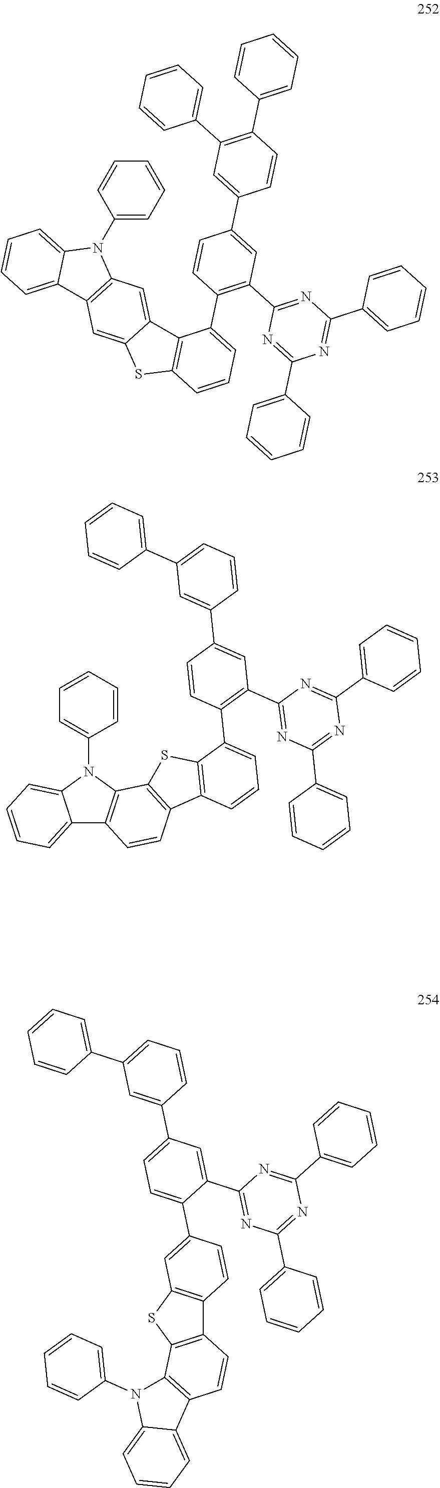 [SMILES: c1ccc(-c2nc(-c3ccccc3)nc(-c3cc(-c4ccc(-c5ccccc5)c(-c5ccccc5)c4)ccc3-c3cccc4sc5cc6c7ccccc7n(-c7ccccc7)c6cc5c34)n2)cc1 ; c1ccc(-c2cccc(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1]

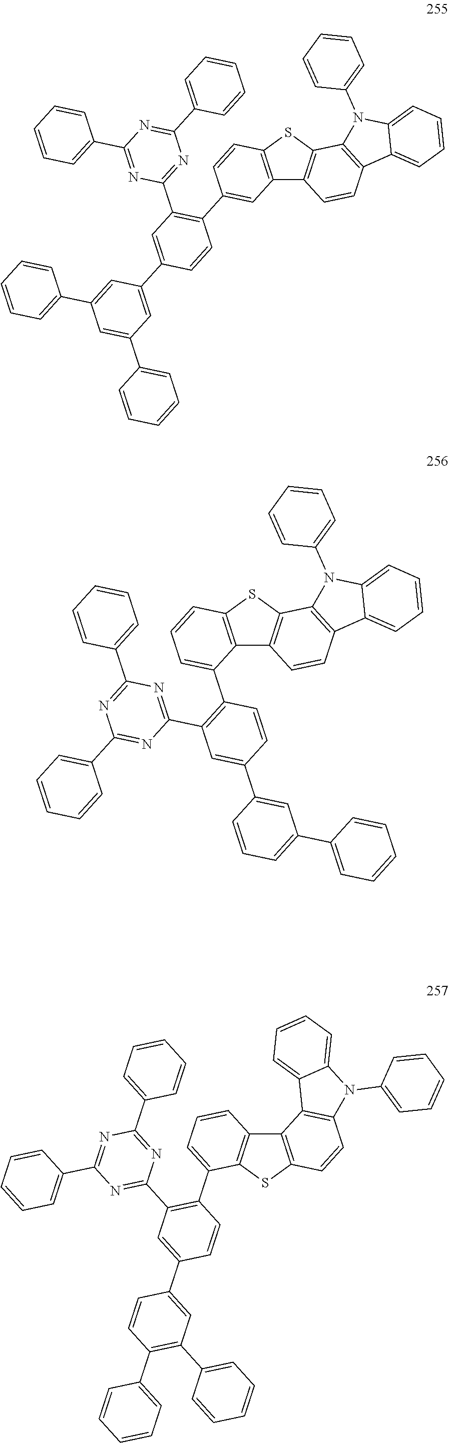 [SMILES: c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3ccc5c(c6ccccc6n5-c5ccccc5)c34)n2)cc1]

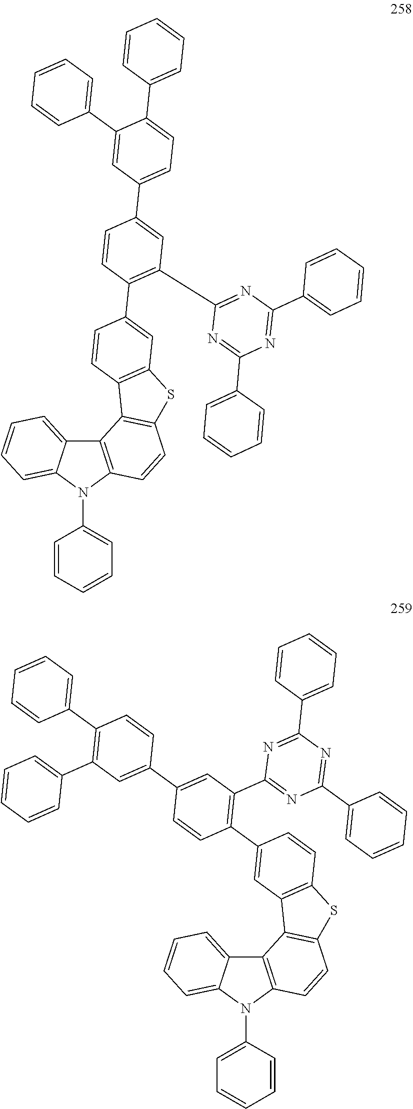 [SMILES: c1ccc(-c2nc(-c3ccccc3)nc(-c3cc(-c4ccc(-c5ccccc5)c(-c5ccccc5)c4)ccc3-c3ccc4c(c3)sc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4sc5ccc6c(c7ccccc7n6-c6ccccc6)c5c4c3)n2)cc1]

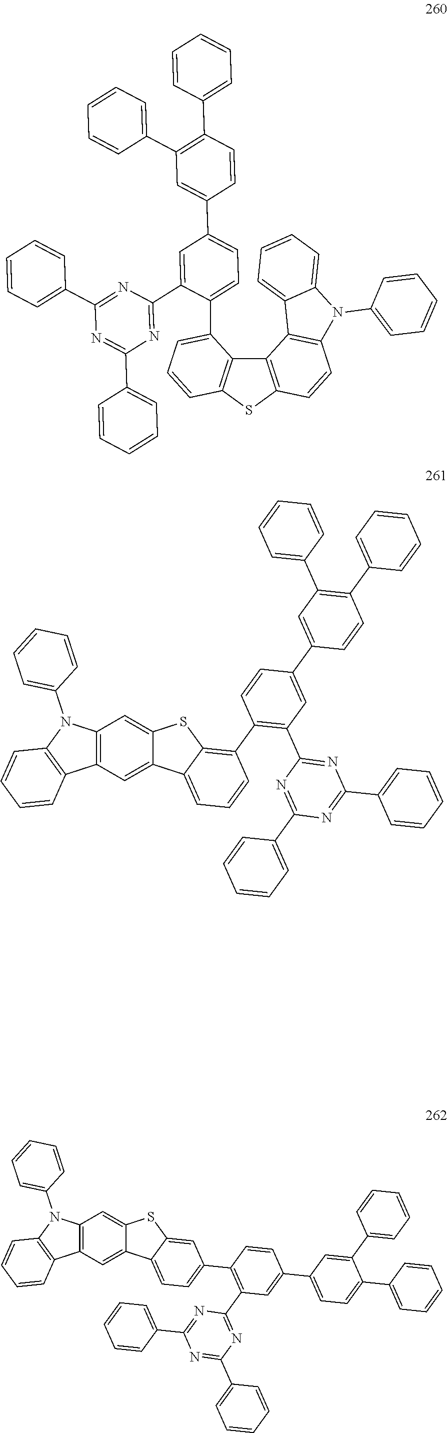 [SMILES: c1ccc(-c2nc(-c3ccccc3)nc(-c3cc(-c4ccc(-c5ccccc5)c(-c5ccccc5)c4)ccc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)sc3cc5c(cc34)c3ccccc3n5-c3ccccc3)n2)cc1]

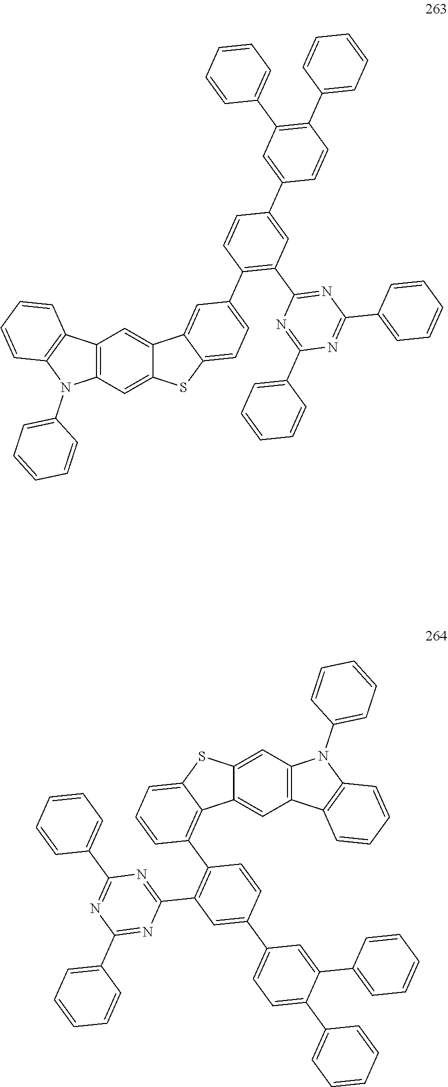 [SMILES: c1ccc(-c2nc(-c3ccccc3)nc(-c3cc(-c4ccc(-c5ccccc5)c(-c5ccccc5)c4)ccc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4sc5cc6c(cc5c34)c3ccccc3n6-c3ccccc3)n2)cc1]

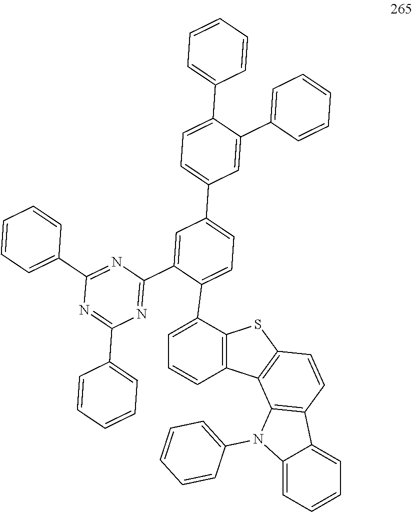 [SMILES: c1ccc(-c2nc(-c3ccccc3)nc(-c3cc(-c4ccc(-c5ccccc5)c(-c5ccccc5)c4)ccc3-c3cccc4c3sc3ccc5c6ccccc6n(-c6ccccc6)c5c34)n2)cc1]

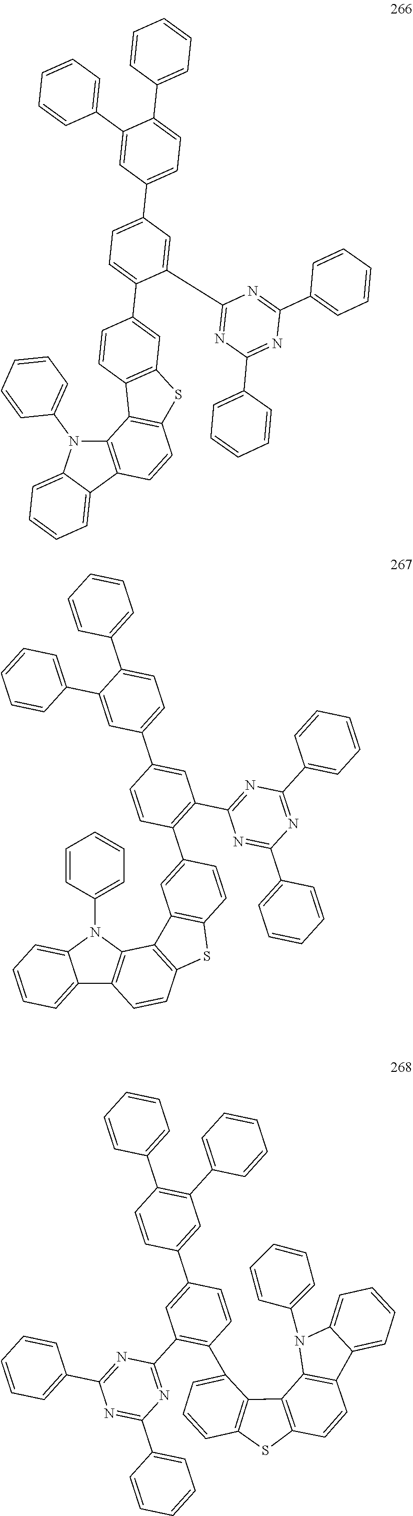 [SMILES: c1ccc(-c2nc(-c3ccccc3)nc(-c3cc(-c4ccc(-c5ccccc5)c(-c5ccccc5)c4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3ccc4sc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)ccc3-c3cccc4sc5ccc6c7ccccc7n(-c7ccccc7)c6c5c34)n2)cc1]

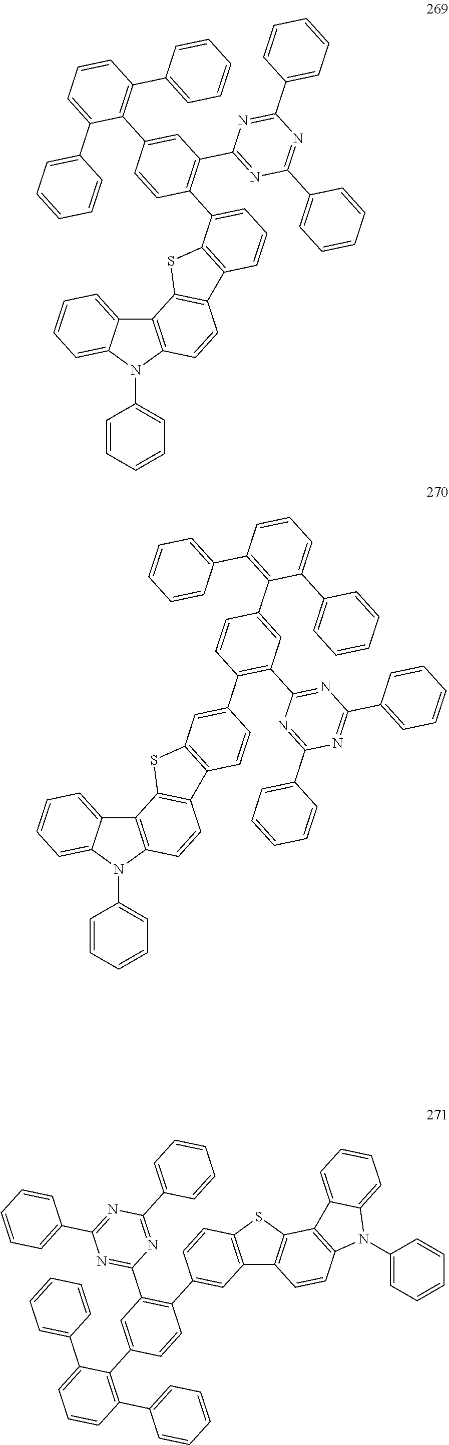 [SMILES: c1ccc(-c2nc(-c3ccccc3)nc(-c3cc(-c4c(-c5ccccc5)cccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4sc5c(ccc6c5c5ccccc5n6-c5ccccc5)c4c3)n2)cc1]

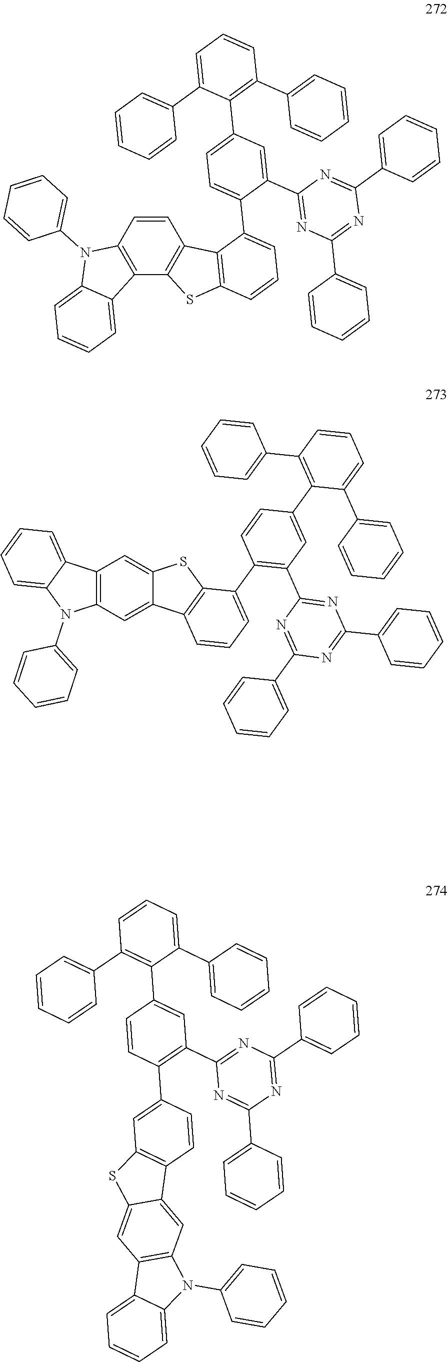 [SMILES: c1ccc(-c2nc(-c3ccccc3)nc(-c3cc(-c4c(-c5ccccc5)cccc4-c4ccccc4)ccc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)ccc3-c3cccc4c3sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4c(c3)sc3cc5c6ccccc6n(-c6ccccc6)c5cc34)n2)cc1]

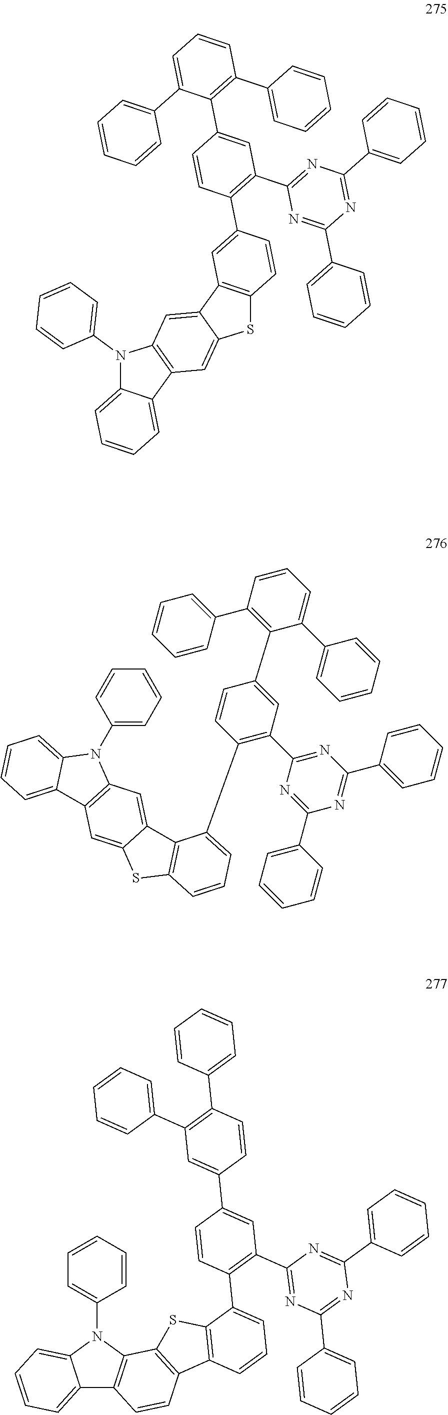 [SMILES: c1ccc(-c2nc(-c3ccccc3)nc(-c3cc(-c4c(-c5ccccc5)cccc4-c4ccccc4)ccc3-c3ccc4sc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)ccc3-c3cccc4sc5cc6c7ccccc7n(-c7ccccc7)c6cc5c34)n2)cc1 ; c1ccc(-c2nc(-c3ccccc3)nc(-c3cc(-c4ccc(-c5ccccc5)c(-c5ccccc5)c4)ccc3-c3cccc4c3sc3c4ccc4c5ccccc5n(-c5ccccc5)c43)n2)cc1]

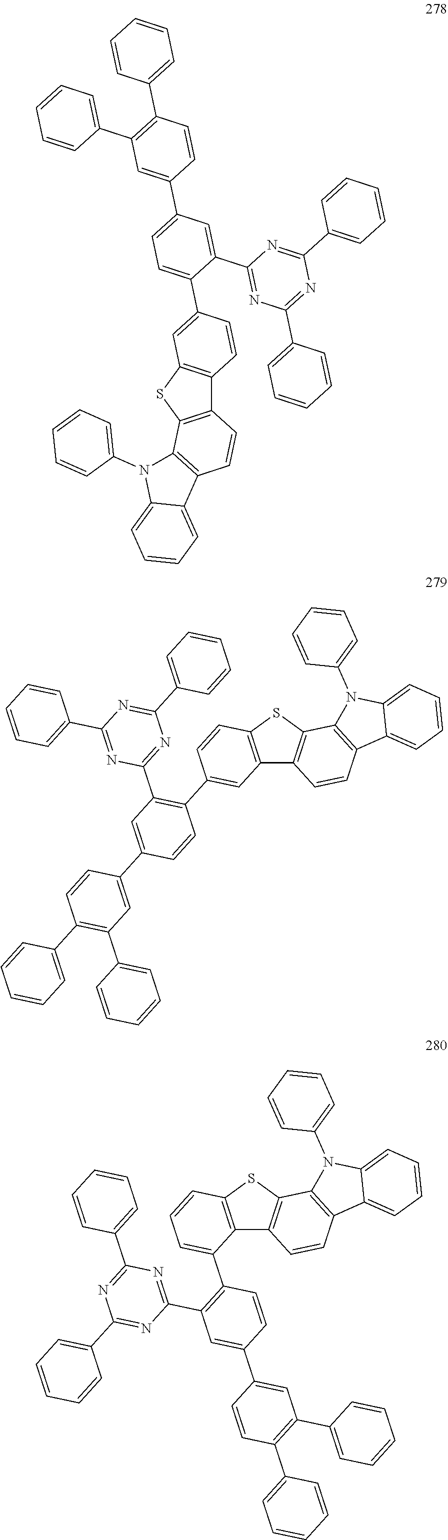 [SMILES: c1ccc(-c2nc(-c3ccccc3)nc(-c3cc(-c4ccc(-c5ccccc5)c(-c5ccccc5)c4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)ccc3-c3cccc4sc5c(ccc6c7ccccc7n(-c7ccccc7)c65)c34)n2)cc1]

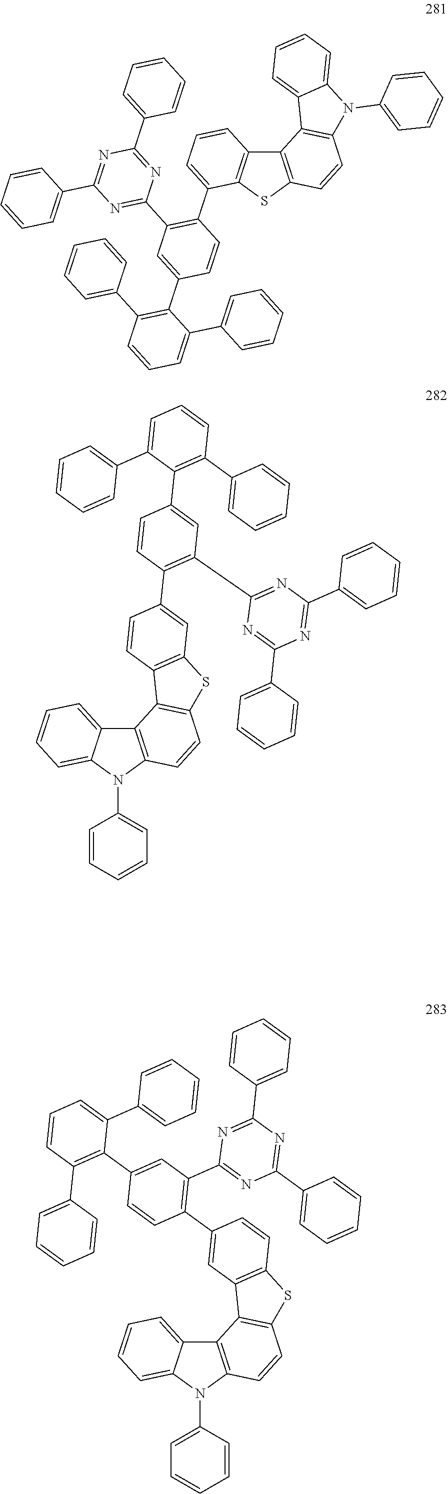

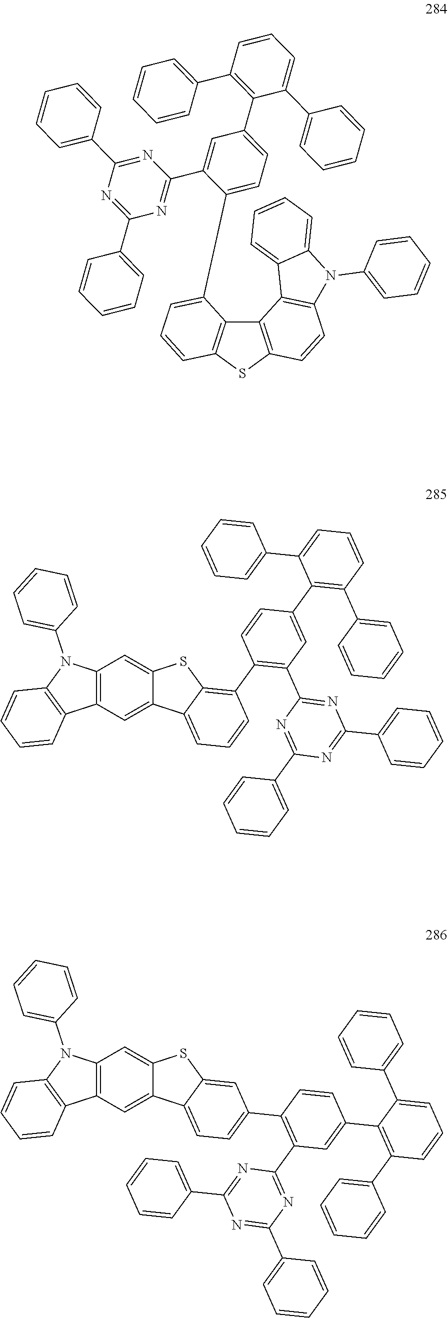 [SMILES: c1ccc(-c2nc(-c3ccccc3)nc(-c3cc(-c4c(-c5ccccc5)cccc4-c4ccccc4)ccc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)ccc3-c3cccc4c3sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)sc3cc5c(cc34)c3ccccc3n5-c3ccccc3)n2)cc1]

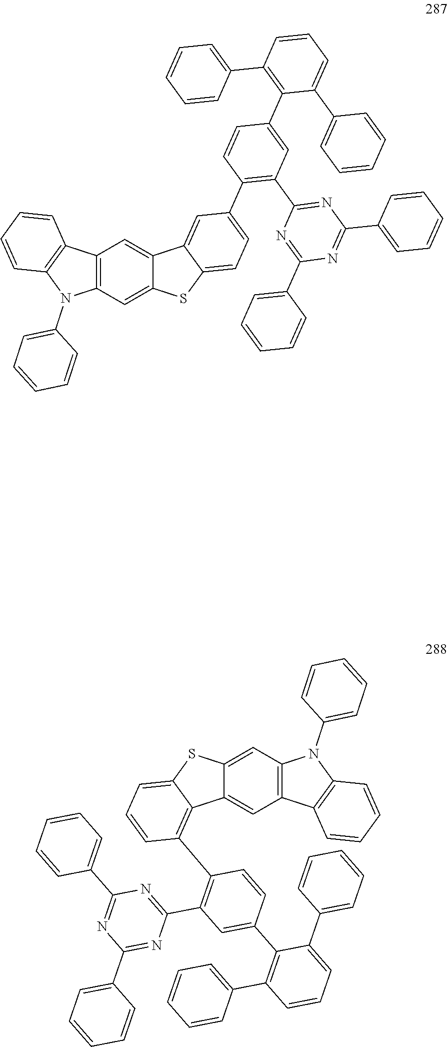 [SMILES: c1ccc(-c2nc(-c3ccccc3)nc(-c3cc(-c4c(-c5ccccc5)cccc4-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)ccc3-c3cccc4sc5cc6c(cc5c34)c3ccccc3n6-c3ccccc3)n2)cc1]

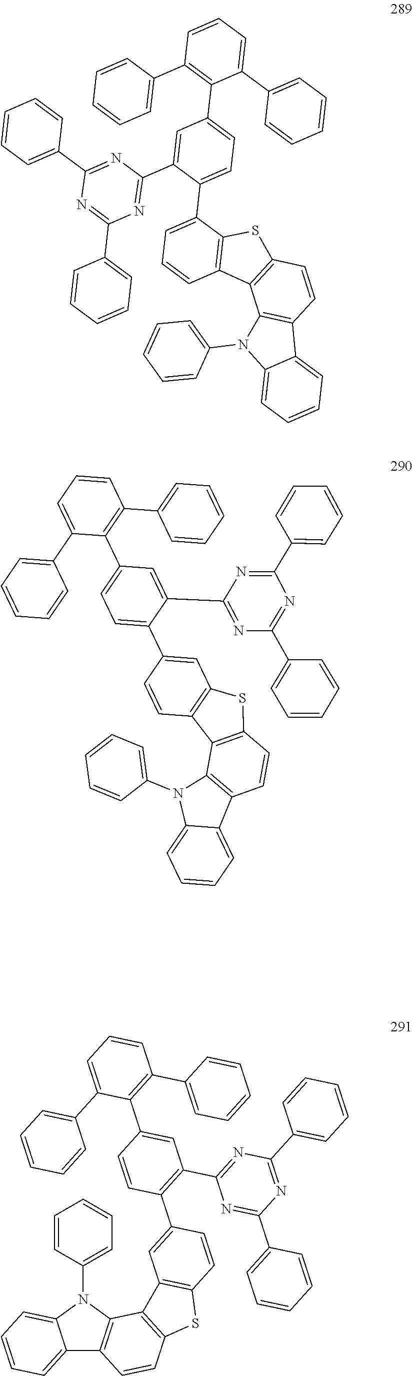 [SMILES: c1ccc(-c2nc(-c3ccccc3)nc(-c3cc(-c4c(-c5ccccc5)cccc4-c4ccccc4)ccc3-c3cccc4c3sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)ccc3-c3ccc4sc5ccc6c7ccccc7n(-c7ccccc7)c6c5c4c3)n2)cc1]

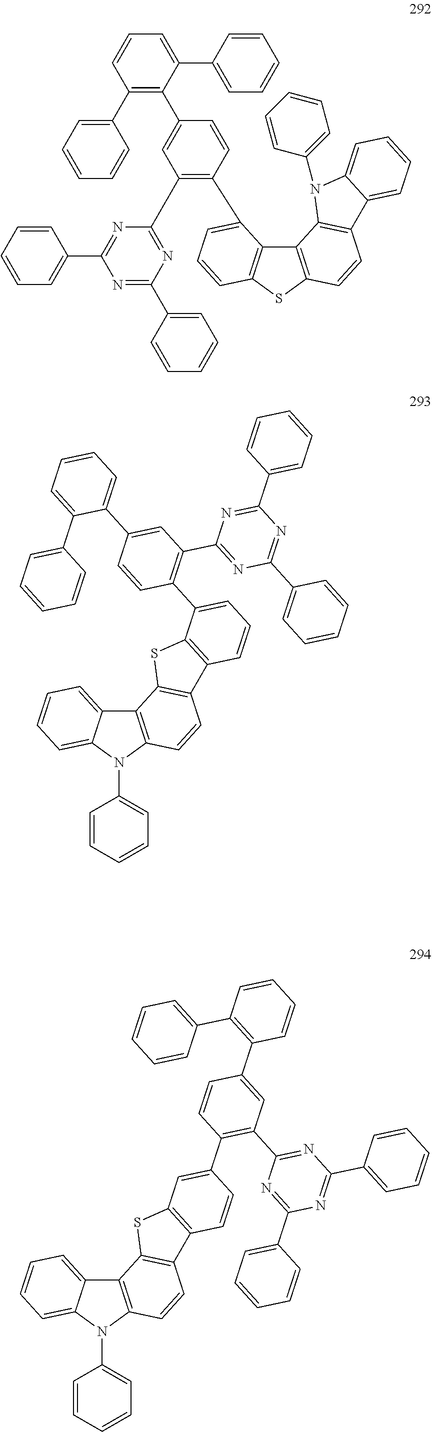 [SMILES: c1ccc(-c2nc(-c3ccccc3)nc(-c3cc(-c4c(-c5ccccc5)cccc4-c4ccccc4)ccc3-c3cccc4sc5ccc6c7ccccc7n(-c7ccccc7)c6c5c34)n2)cc1 ; c1ccc(-c2nc(-c3ccccc3)nc(-c3cc(-c4ccccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)ccc3-c3ccc4c(c3)sc3c4ccc4c3c3ccccc3n4-c3ccccc3)n2)cc1]

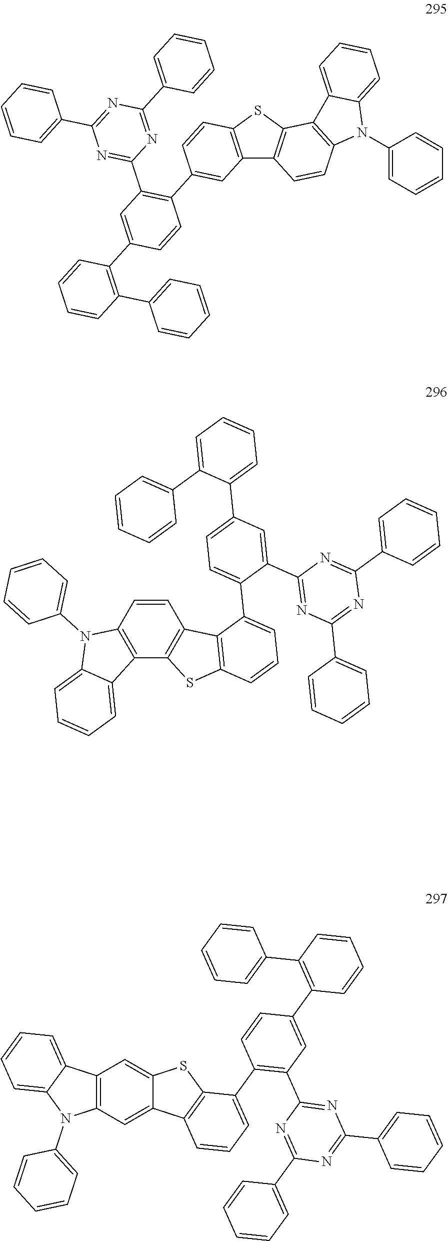 [SMILES: c1ccc(-c2nc(-c3ccccc3)nc(-c3cc(-c4ccccc4-c4ccccc4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)ccc3-c3cccc4sc5c(ccc6c5c5ccccc5n6-c5ccccc5)c34)n2)cc1 ; c1ccc(-c2nc(-c3ccccc3)nc(-c3cc(-c4ccccc4-c4ccccc4)ccc3-c3cccc4c3sc3cc5c6ccccc6n(-c6ccccc6)c5cc34)n2)cc1]

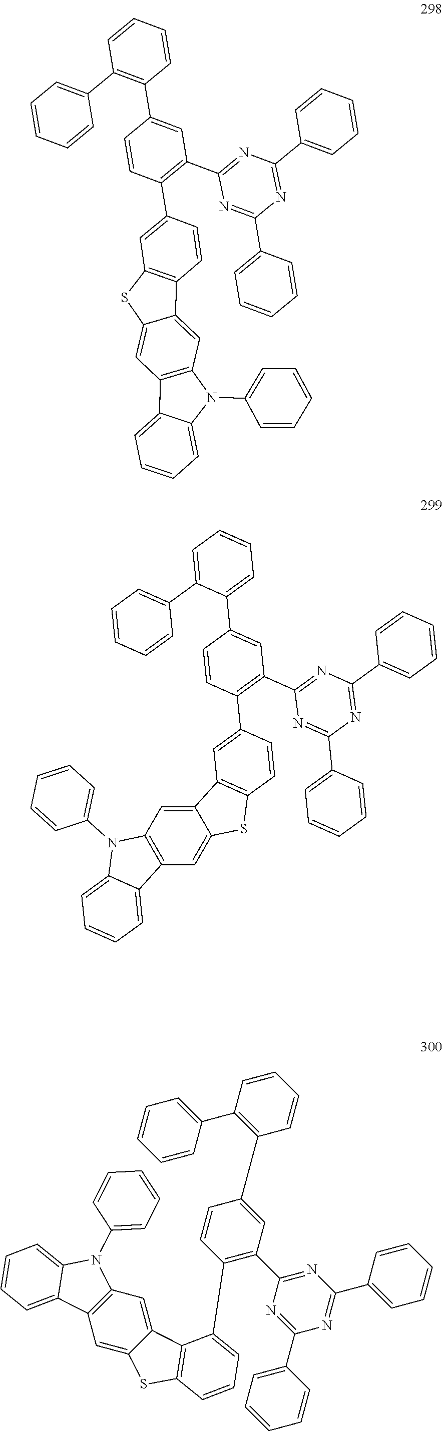 [SMILES: c1ccc(-c2nc(-c3ccccc3)nc(-c3cc(-c4ccccc4-c4ccccc4)ccc3-c3ccc4c(c3)sc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)ccc3-c3cccc4sc5cc6c7ccccc7n(-c7ccccc7)c6cc5c34)n2)cc1]

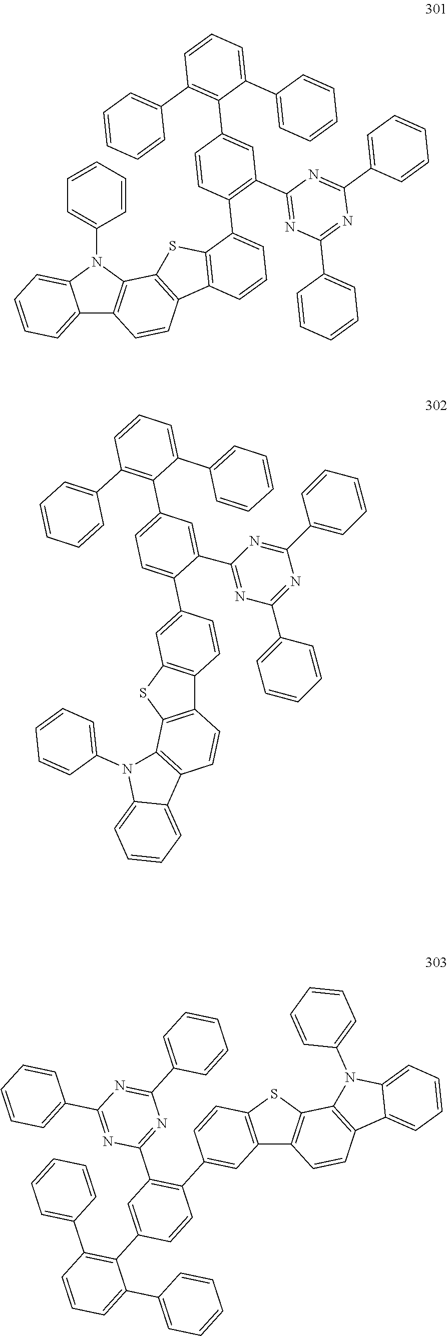 [SMILES: c1ccc(-c2nc(-c3ccccc3)nc(-c3cc(-c4c(-c5ccccc5)cccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4sc5c(ccc6c7ccccc7n(-c7ccccc7)c65)c4c3)n2)cc1]

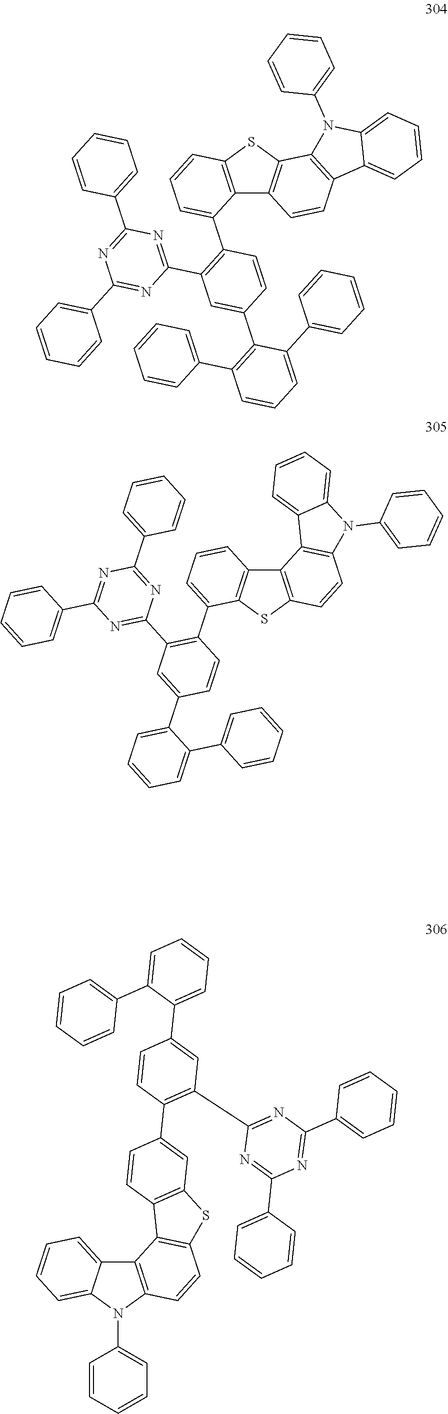 [SMILES: c1ccc(-c2nc(-c3ccccc3)nc(-c3cc(-c4c(-c5ccccc5)cccc4-c4ccccc4)ccc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)ccc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)ccc3-c3ccc4c(c3)sc3ccc5c(c6ccccc6n5-c5ccccc5)c34)n2)cc1]

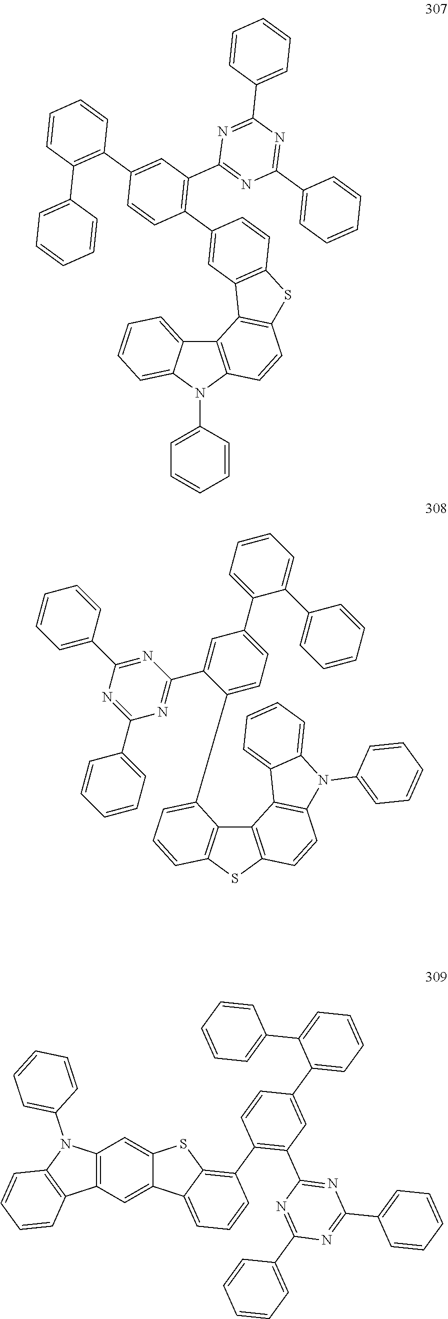 [SMILES: c1ccc(-c2nc(-c3ccccc3)nc(-c3cc(-c4ccccc4-c4ccccc4)ccc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)ccc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)ccc3-c3cccc4c3sc3cc5c(cc34)c3ccccc3n5-c3ccccc3)n2)cc1]

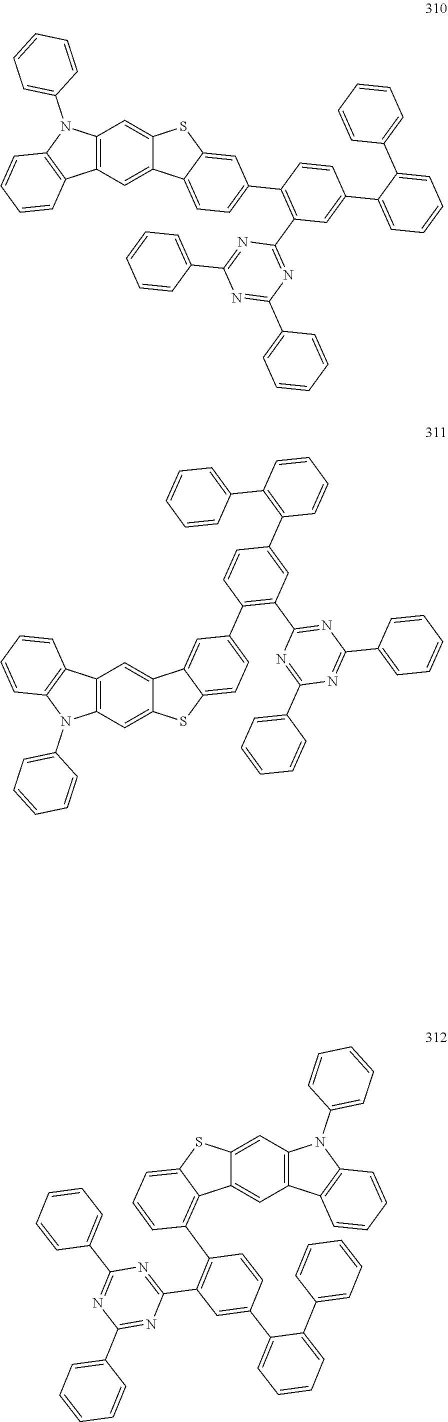 [SMILES: c1ccc(-c2nc(-c3ccccc3)nc(-c3cc(-c4ccccc4-c4ccccc4)ccc3-c3ccc4c(c3)sc3cc5c(cc34)c3ccccc3n5-c3ccccc3)n2)cc1 ; c1ccc(-c2nc(-c3ccccc3)nc(-c3cc(-c4ccccc4-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)ccc3-c3cccc4sc5cc6c(cc5c34)c3ccccc3n6-c3ccccc3)n2)cc1]

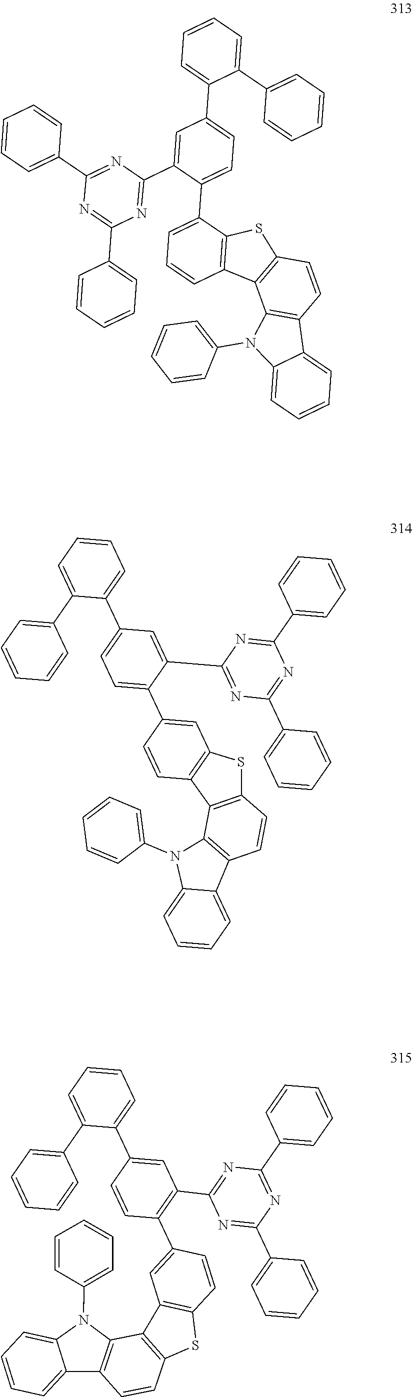 [SMILES: c1ccc(-c2nc(-c3ccccc3)nc(-c3cc(-c4ccccc4-c4ccccc4)ccc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4sc5ccc6c7ccccc7n(-c7ccccc7)c6c5c4c3)n2)cc1]

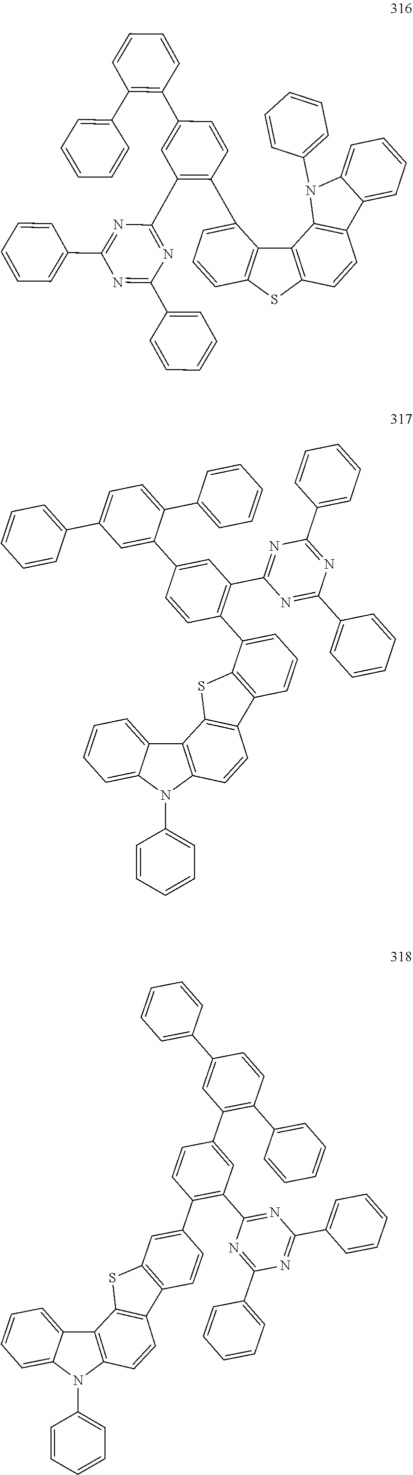 [SMILES: c1ccc(-c2nc(-c3ccccc3)nc(-c3cc(-c4ccccc4-c4ccccc4)ccc3-c3cccc4sc5ccc6c7ccccc7n(-c7ccccc7)c6c5c34)n2)cc1 ; c1ccc(-c2ccc(-c3ccccc3)c(-c3ccc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1]

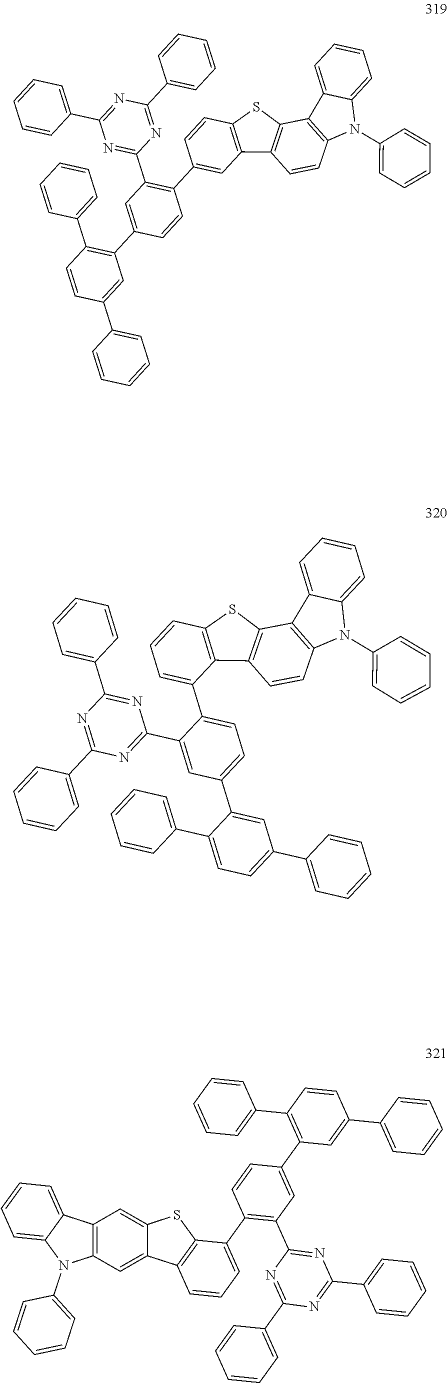 [SMILES: c1ccc(-c2ccc(-c3ccccc3)c(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4cc6c7ccccc7n(-c7ccccc7)c6cc45)c(-c4nc(-c5ccccc5)nc(-c5ccccc5)n4)c3)c2)cc1]

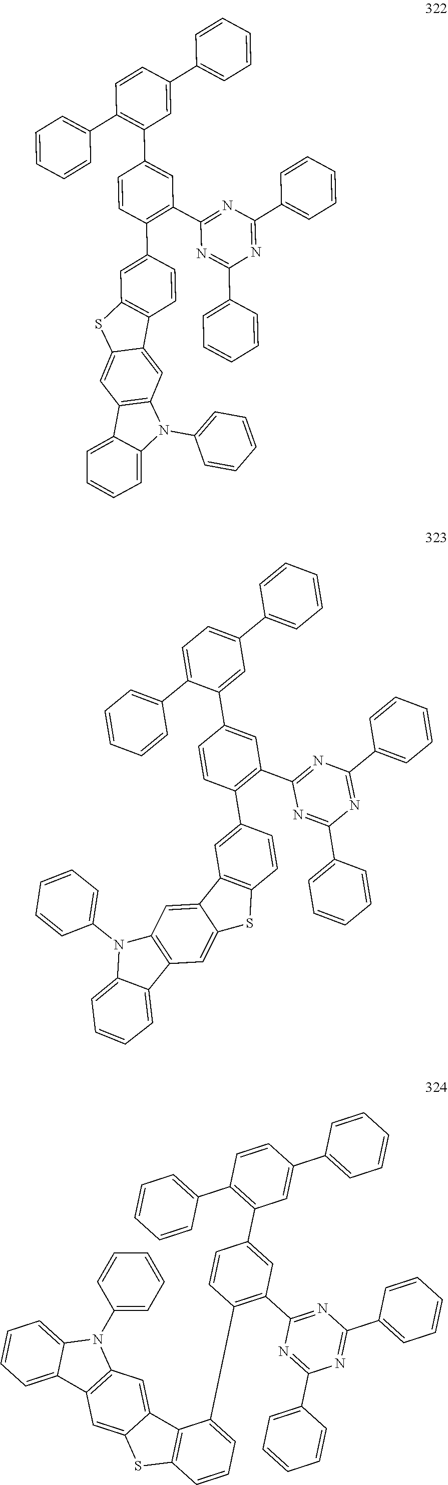 [SMILES: c1ccc(-c2ccc(-c3ccccc3)c(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6cc7c8ccccc8n(-c8ccccc8)c7cc6c45)c(-c4nc(-c5ccccc5)nc(-c5ccccc5)n4)c3)c2)cc1]

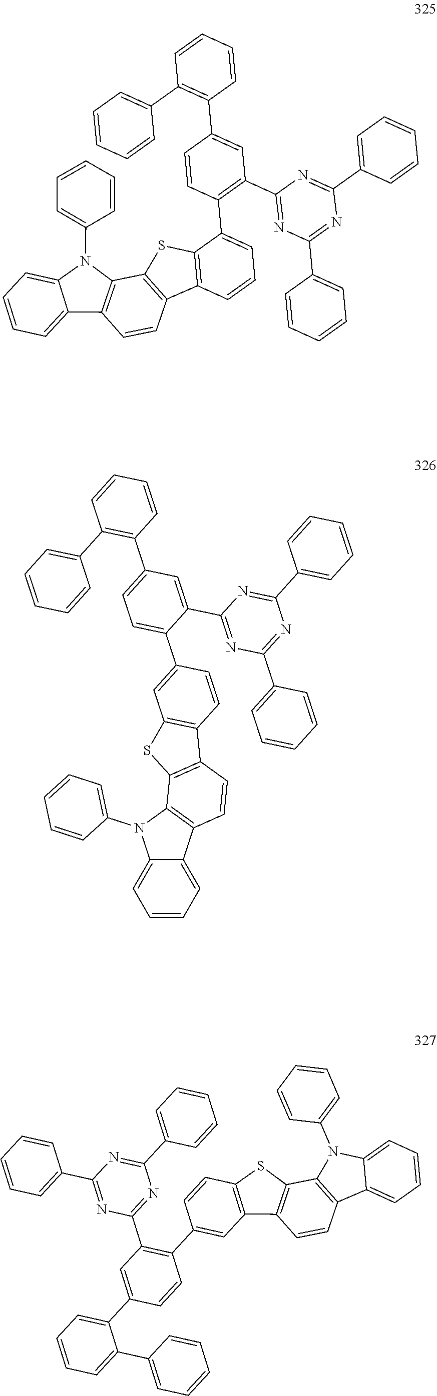 [SMILES: c1ccc(-c2nc(-c3ccccc3)nc(-c3cc(-c4ccccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4sc5c(ccc6c7ccccc7n(-c7ccccc7)c65)c4c3)n2)cc1]

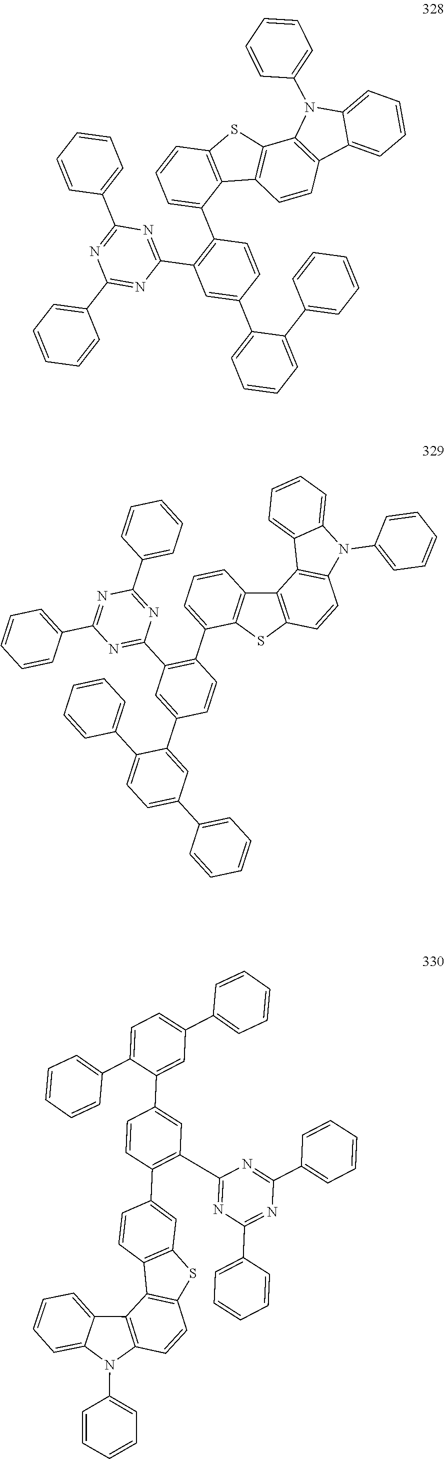 [SMILES: c1ccc(-c2nc(-c3ccccc3)nc(-c3cc(-c4ccccc4-c4ccccc4)ccc3-c3cccc4sc5c(ccc6c7ccccc7n(-c7ccccc7)c65)c34)n2)cc1 ; c1ccc(-c2ccc(-c3ccccc3)c(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1]

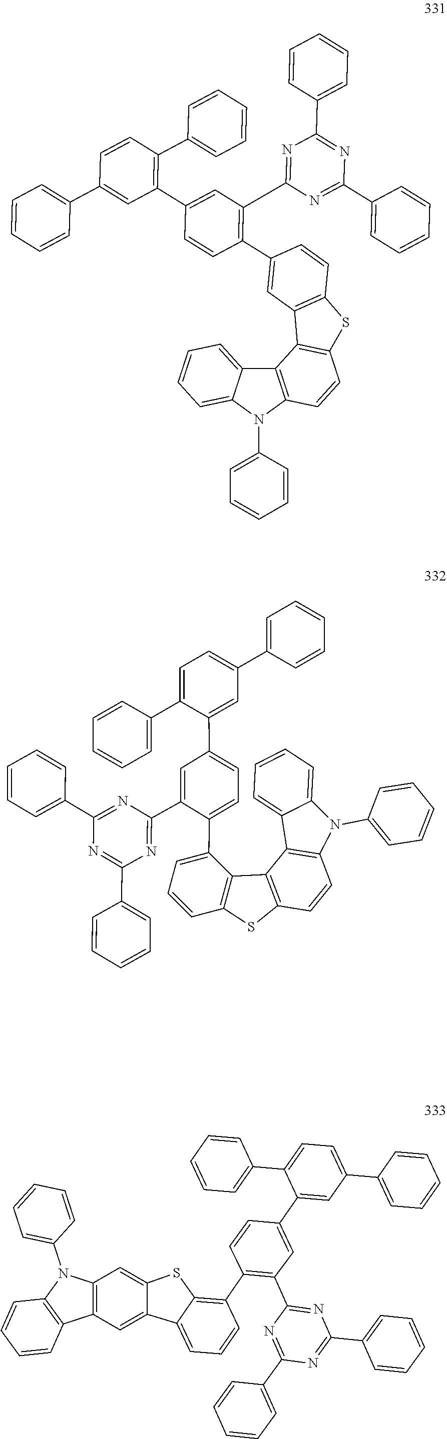 [SMILES: c1ccc(-c2ccc(-c3ccccc3)c(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4cc6c(cc45)c4ccccc4n6-c4ccccc4)c(-c4nc(-c5ccccc5)nc(-c5ccccc5)n4)c3)c2)cc1]

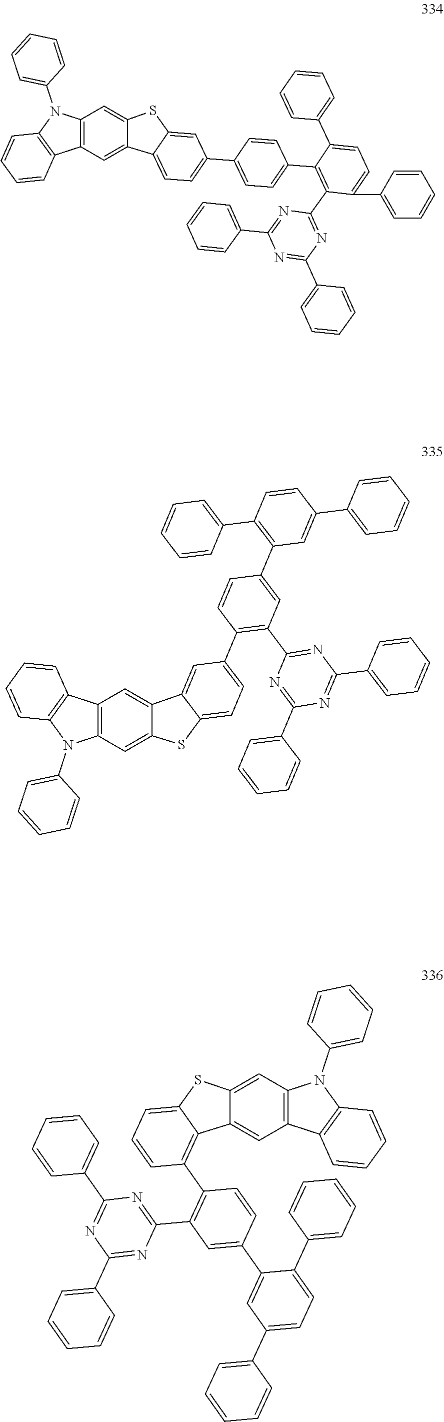 [SMILES: c1ccc(-c2nc(-c3ccccc3)nc(-c3c(-c4ccccc4)ccc(-c4ccccc4)c3-c3ccc(-c4ccc5c(c4)sc4cc6c(cc45)c4ccccc4n6-c4ccccc4)cc3)n2)cc1 ; c1ccc(-c2ccc(-c3ccccc3)c(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5sc6cc7c(cc6c45)c4ccccc4n7-c4ccccc4)c(-c4nc(-c5ccccc5)nc(-c5ccccc5)n4)c3)c2)cc1]

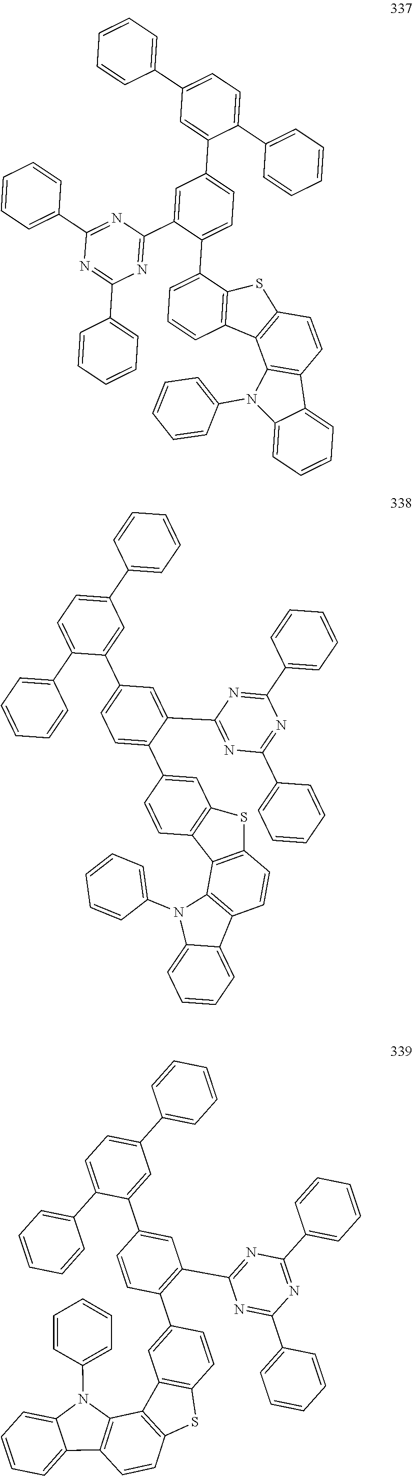 [SMILES: c1ccc(-c2ccc(-c3ccccc3)c(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1]

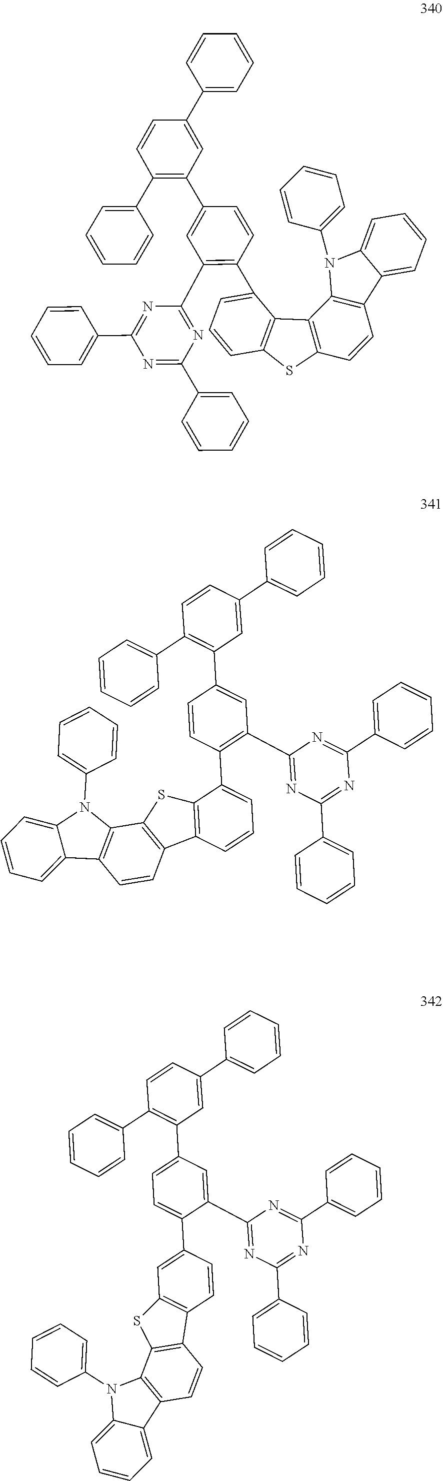 [SMILES: c1ccc(-c2ccc(-c3ccccc3)c(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1]

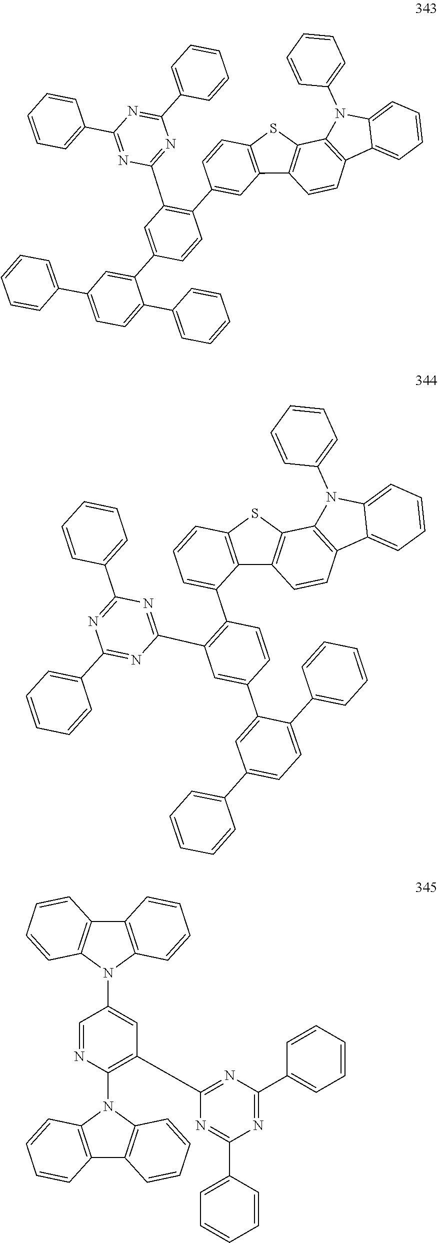 [SMILES: c1ccc(-c2ccc(-c3ccccc3)c(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-n4c5ccccc5c5ccccc54)cnc3-n3c4ccccc4c4ccccc43)n2)cc1]

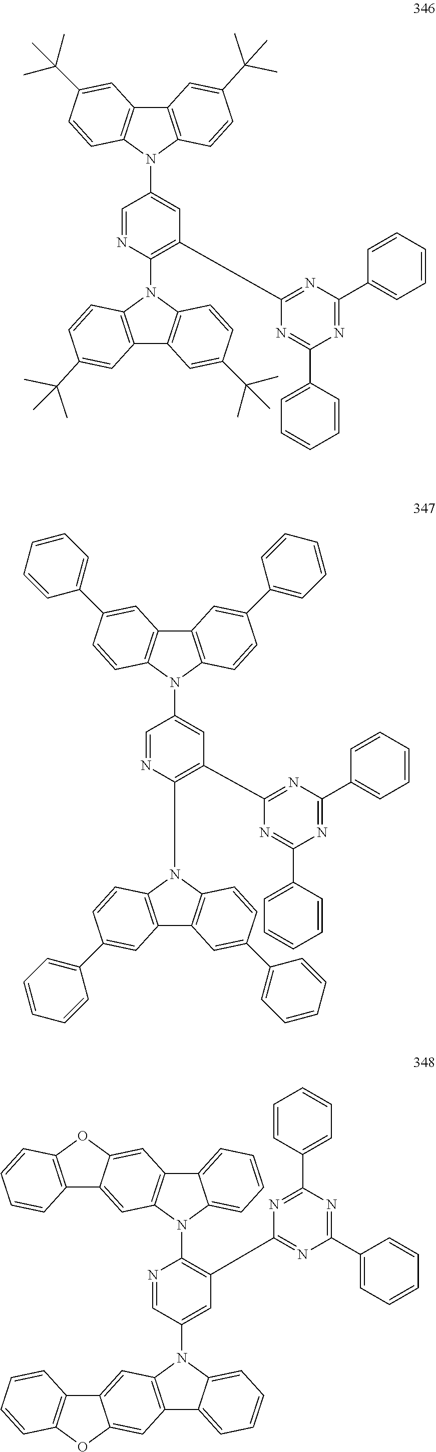 [SMILES: CC(C)(C)c1ccc2c(c1)c1cc(C(C)(C)C)ccc1n2-c1cnc(-n2c3ccc(C(C)(C)C)cc3c3cc(C(C)(C)C)ccc32)c(-c2nc(-c3ccccc3)nc(-c3ccccc3)n2)c1 ; c1ccc(-c2ccc3c(c2)c2cc(-c4ccccc4)ccc2n3-c2cnc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2nc(-c3ccccc3)nc(-c3cc(-n4c5ccccc5c5cc6oc7ccccc7c6cc54)cnc3-n3c4ccccc4c4cc5oc6ccccc6c5cc43)n2)cc1]

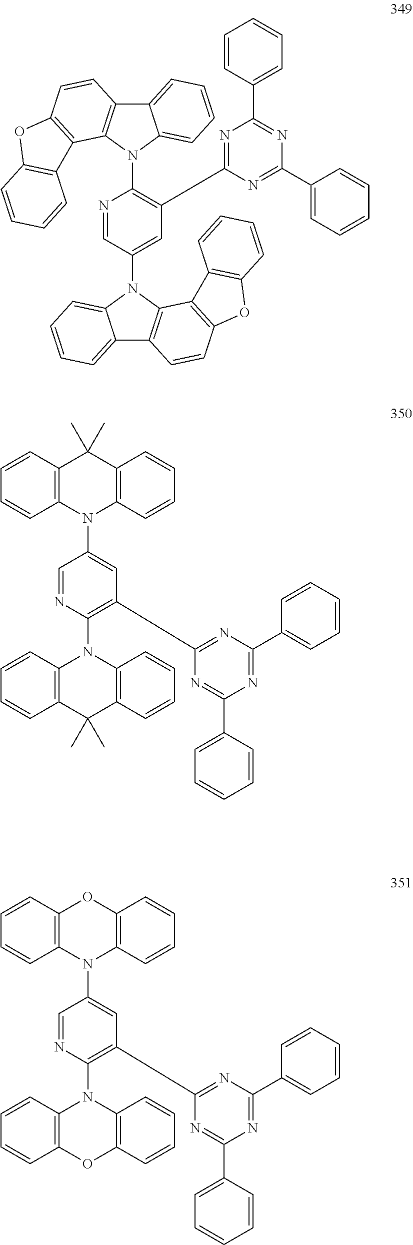 [SMILES: c1ccc(-c2nc(-c3ccccc3)nc(-c3cc(-n4c5ccccc5c5ccc6oc7ccccc7c6c54)cnc3-n3c4ccccc4c4ccc5oc6ccccc6c5c43)n2)cc1 ; CC1(C)c2ccccc2N(c2cnc(N3c4ccccc4C(C)(C)c4ccccc43)c(-c3nc(-c4ccccc4)nc(-c4ccccc4)n3)c2)c2ccccc21 ; c1ccc(-c2nc(-c3ccccc3)nc(-c3cc(N4c5ccccc5Oc5ccccc54)cnc3N3c4ccccc4Oc4ccccc43)n2)cc1]

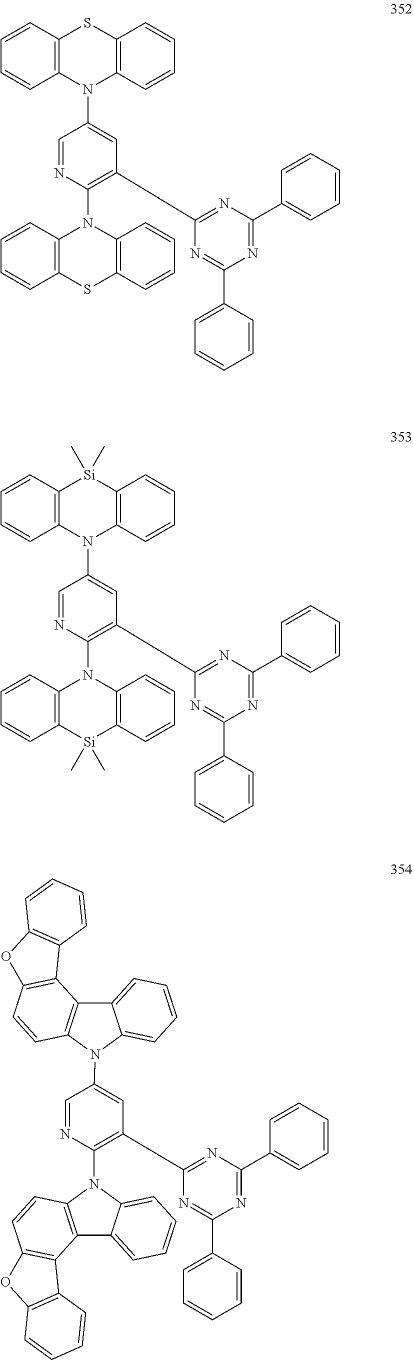 [SMILES: c1ccc(-c2nc(-c3ccccc3)nc(-c3cc(N4c5ccccc5Sc5ccccc54)cnc3N3c4ccccc4Sc4ccccc43)n2)cc1 ; C[Si]1(C)c2ccccc2N(c2cnc(N3c4ccccc4[Si](C)(C)c4ccccc43)c(-c3nc(-c4ccccc4)nc(-c4ccccc4)n3)c2)c2ccccc21 ; c1ccc(-c2nc(-c3ccccc3)nc(-c3cc(-n4c5ccccc5c5c6c(ccc54)oc4ccccc46)cnc3-n3c4ccccc4c4c5c(ccc43)oc3ccccc35)n2)cc1]

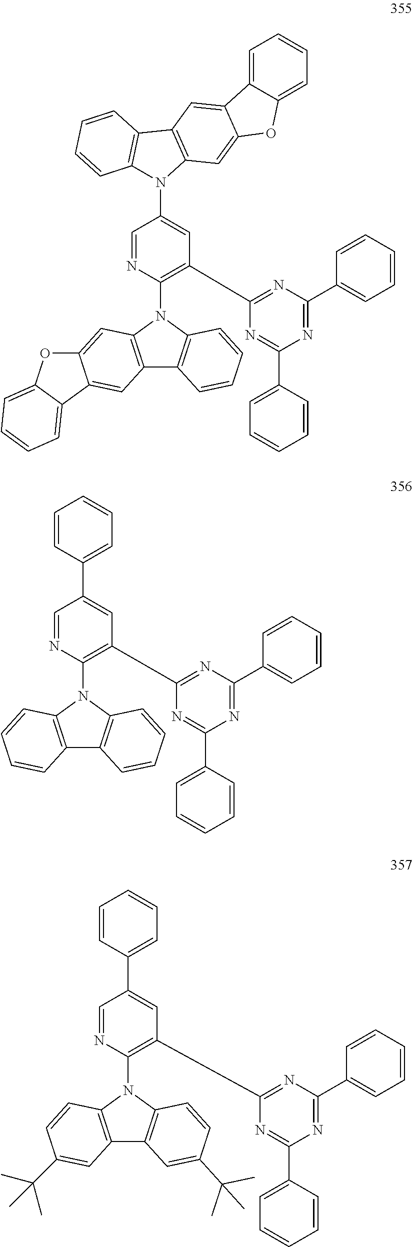 [SMILES: c1ccc(-c2nc(-c3ccccc3)nc(-c3cc(-n4c5ccccc5c5cc6c(cc54)oc4ccccc46)cnc3-n3c4ccccc4c4cc5c(cc43)oc3ccccc35)n2)cc1 ; c1ccc(-c2cnc(-n3c4ccccc4c4ccccc43)c(-c3nc(-c4ccccc4)nc(-c4ccccc4)n3)c2)cc1 ; CC(C)(C)c1ccc2c(c1)c1cc(C(C)(C)C)ccc1n2-c1ncc(-c2ccccc2)cc1-c1nc(-c2ccccc2)nc(-c2ccccc2)n1]

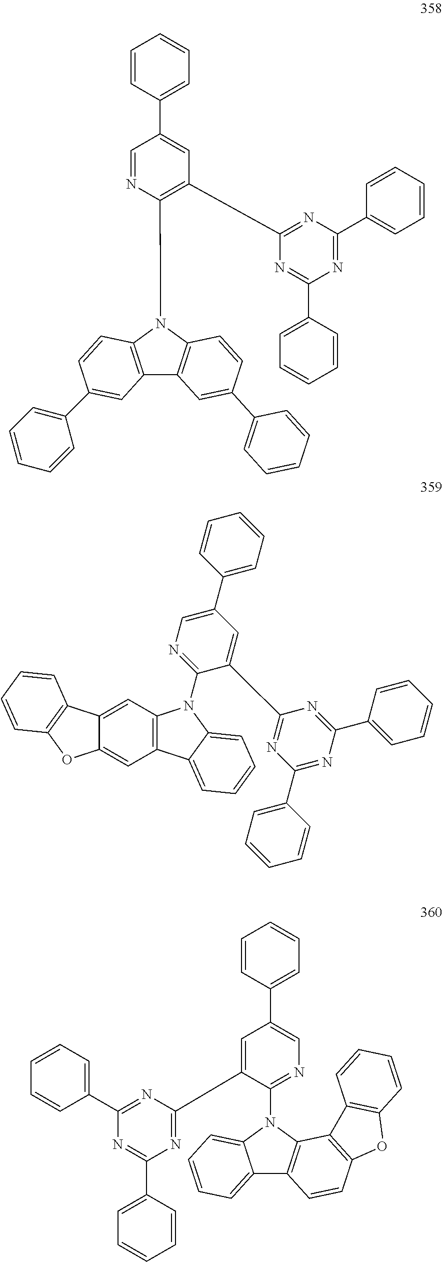

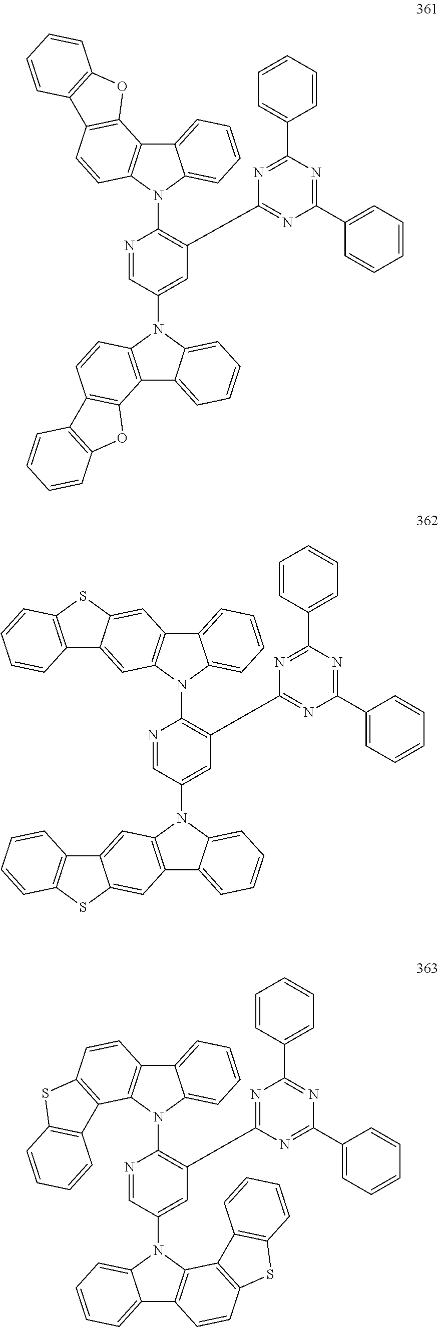 [SMILES: c1ccc(-c2nc(-c3ccccc3)nc(-c3cc(-n4c5ccccc5c5c6oc7ccccc7c6ccc54)cnc3-n3c4ccccc4c4c5oc6ccccc6c5ccc43)n2)cc1 ; c1ccc(-c2nc(-c3ccccc3)nc(-c3cc(-n4c5ccccc5c5cc6sc7ccccc7c6cc54)cnc3-n3c4ccccc4c4cc5sc6ccccc6c5cc43)n2)cc1 ; c1ccc(-c2nc(-c3ccccc3)nc(-c3cc(-n4c5ccccc5c5ccc6sc7ccccc7c6c54)cnc3-n3c4ccccc4c4ccc5sc6ccccc6c5c43)n2)cc1]

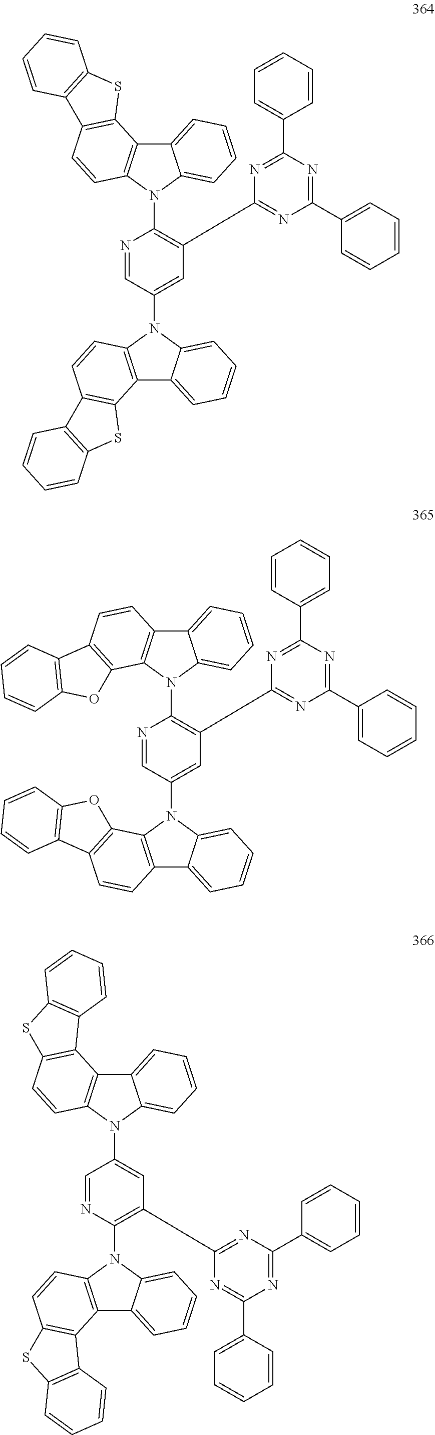 [SMILES: c1ccc(-c2nc(-c3ccccc3)nc(-c3cc(-n4c5ccccc5c5c6sc7ccccc7c6ccc54)cnc3-n3c4ccccc4c4c5sc6ccccc6c5ccc43)n2)cc1 ; c1ccc(-c2nc(-c3ccccc3)nc(-c3cc(-n4c5ccccc5c5ccc6c7ccccc7oc6c54)cnc3-n3c4ccccc4c4ccc5c6ccccc6oc5c43)n2)cc1 ; c1ccc(-c2nc(-c3ccccc3)nc(-c3cc(-n4c5ccccc5c5c6c(ccc54)sc4ccccc46)cnc3-n3c4ccccc4c4c5c(ccc43)sc3ccccc35)n2)cc1]

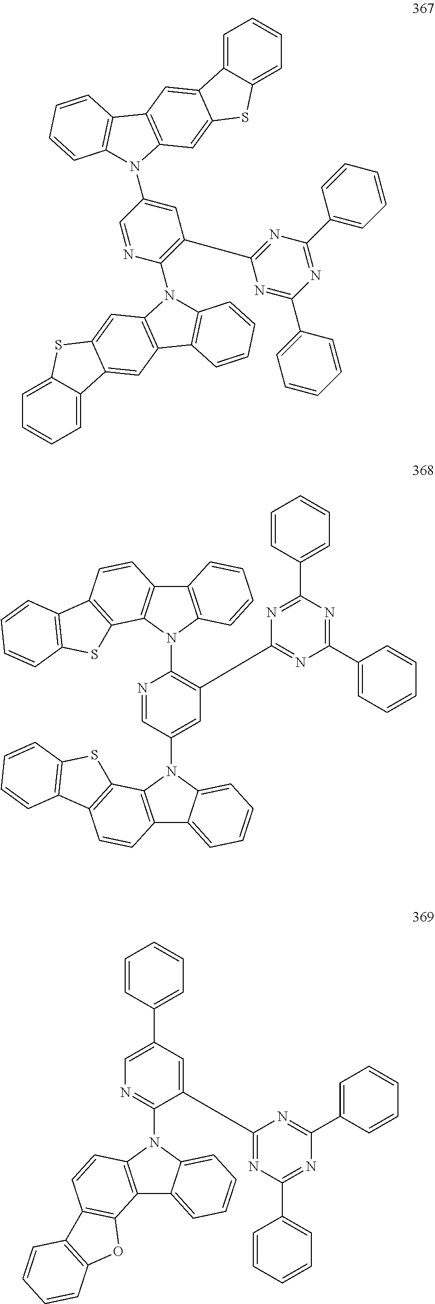 [SMILES: c1ccc(-c2nc(-c3ccccc3)nc(-c3cc(-n4c5ccccc5c5cc6c(cc54)sc4ccccc46)cnc3-n3c4ccccc4c4cc5c(cc43)sc3ccccc35)n2)cc1 ; c1ccc(-c2nc(-c3ccccc3)nc(-c3cc(-n4c5ccccc5c5ccc6c7ccccc7sc6c54)cnc3-n3c4ccccc4c4ccc5c6ccccc6sc5c43)n2)cc1 ; c1ccc(-c2cnc(-n3c4ccccc4c4c5oc6ccccc6c5ccc43)c(-c3nc(-c4ccccc4)nc(-c4ccccc4)n3)c2)cc1]

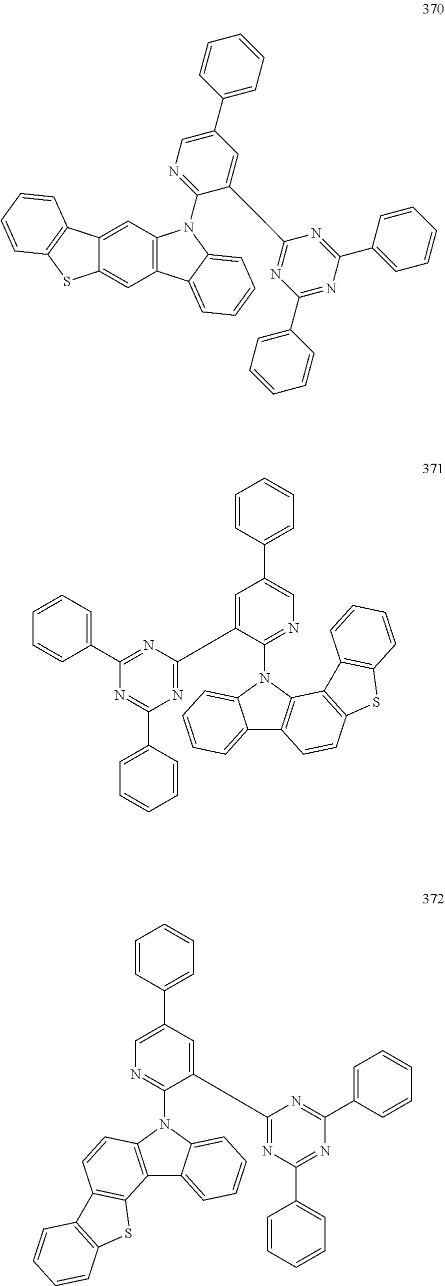 [SMILES: c1ccc(-c2cnc(-n3c4ccccc4c4cc5sc6ccccc6c5cc43)c(-c3nc(-c4ccccc4)nc(-c4ccccc4)n3)c2)cc1 ; c1ccc(-c2cnc(-n3c4ccccc4c4ccc5sc6ccccc6c5c43)c(-c3nc(-c4ccccc4)nc(-c4ccccc4)n3)c2)cc1 ; c1ccc(-c2cnc(-n3c4ccccc4c4c5sc6ccccc6c5ccc43)c(-c3nc(-c4ccccc4)nc(-c4ccccc4)n3)c2)cc1]

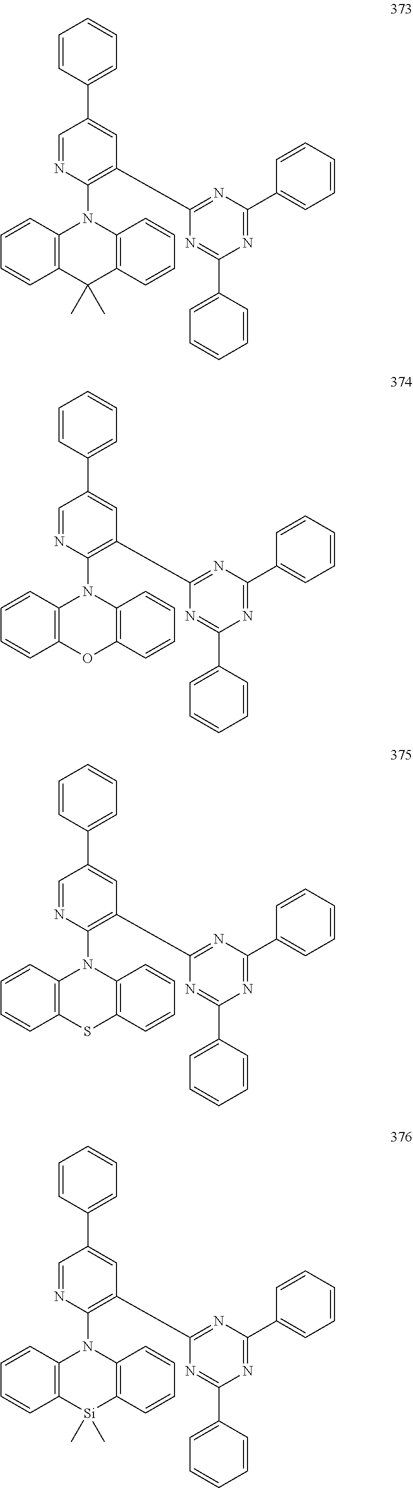 [SMILES: CC1(C)c2ccccc2N(c2ncc(-c3ccccc3)cc2-c2nc(-c3ccccc3)nc(-c3ccccc3)n2)c2ccccc21 ; c1ccc(-c2cnc(N3c4ccccc4Oc4ccccc43)c(-c3nc(-c4ccccc4)nc(-c4ccccc4)n3)c2)cc1 ; c1ccc(-c2cnc(N3c4ccccc4Sc4ccccc43)c(-c3nc(-c4ccccc4)nc(-c4ccccc4)n3)c2)cc1 ; C[Si]1(C)c2ccccc2N(c2ncc(-c3ccccc3)cc2-c2nc(-c3ccccc3)nc(-c3ccccc3)n2)c2ccccc21]

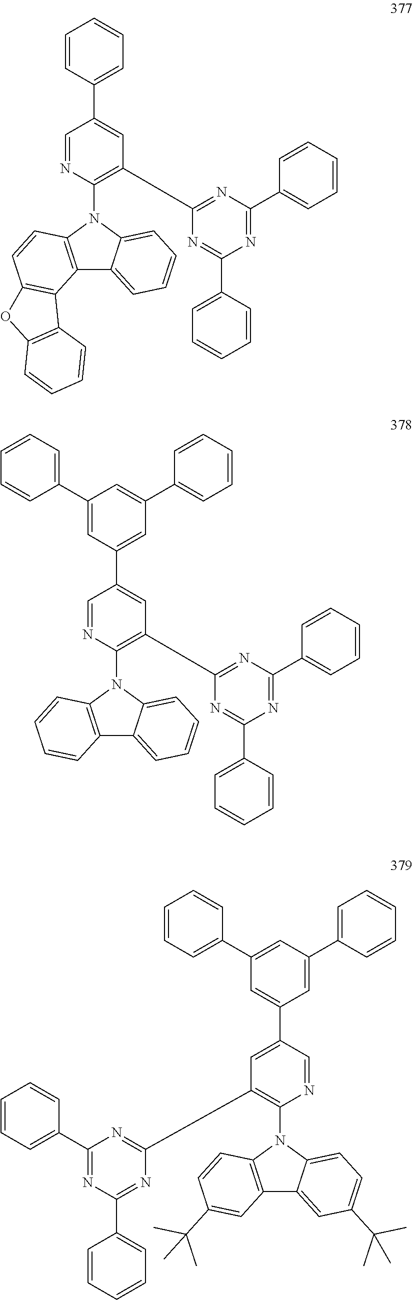

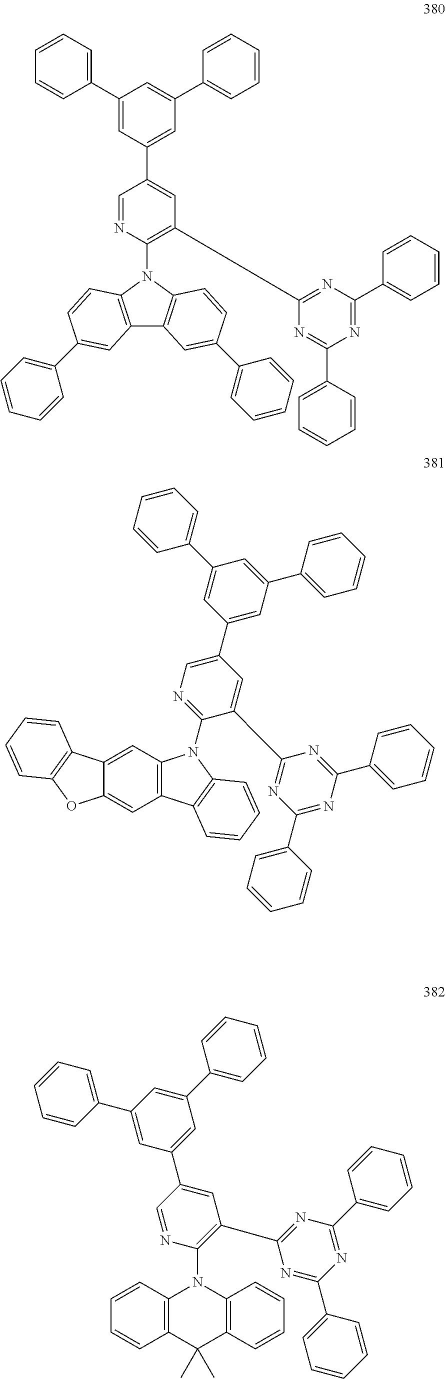 [SMILES: c1ccc(-c2cc(-c3ccccc3)cc(-c3cnc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6oc7ccccc7c6cc54)c(-c4nc(-c5ccccc5)nc(-c5ccccc5)n4)c3)c2)cc1 ; CC1(C)c2ccccc2N(c2ncc(-c3cc(-c4ccccc4)cc(-c4ccccc4)c3)cc2-c2nc(-c3ccccc3)nc(-c3ccccc3)n2)c2ccccc21]

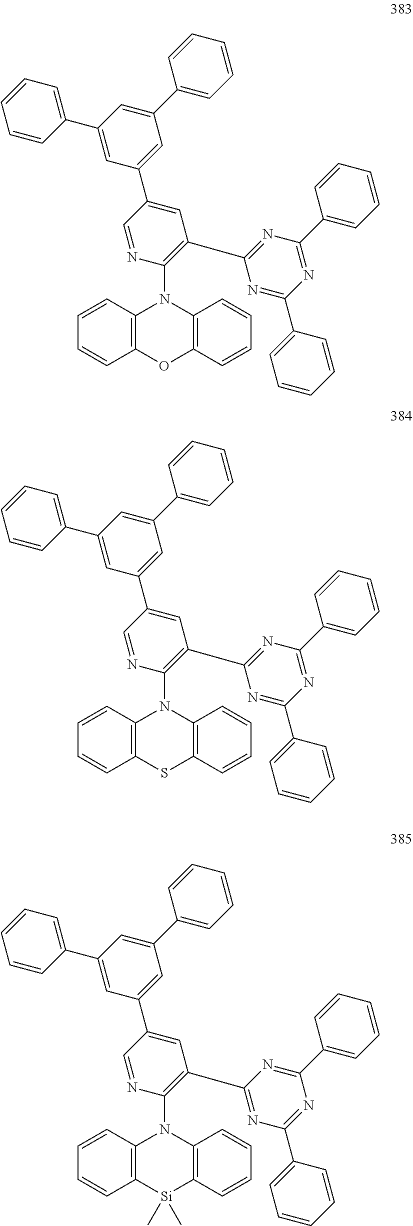 [SMILES: c1ccc(-c2cc(-c3ccccc3)cc(-c3cnc(N4c5ccccc5Oc5ccccc54)c(-c4nc(-c5ccccc5)nc(-c5ccccc5)n4)c3)c2)cc1 ; c1ccc(-c2cc(-c3ccccc3)cc(-c3cnc(N4c5ccccc5Sc5ccccc54)c(-c4nc(-c5ccccc5)nc(-c5ccccc5)n4)c3)c2)cc1 ; C[Si]1(C)c2ccccc2N(c2ncc(-c3cc(-c4ccccc4)cc(-c4ccccc4)c3)cc2-c2nc(-c3ccccc3)nc(-c3ccccc3)n2)c2ccccc21]

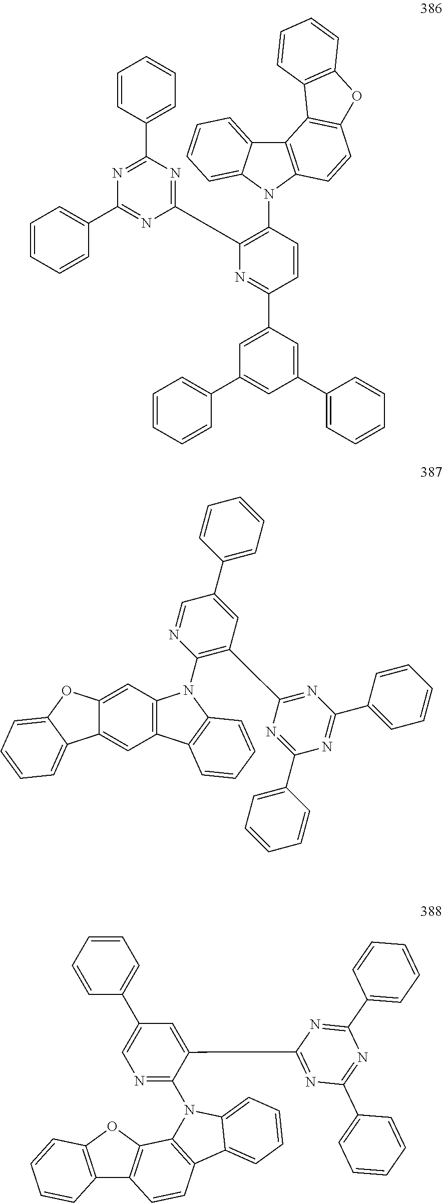 [SMILES: c1ccc(-c2cc(-c3ccccc3)cc(-c3ccc(-n4c5ccccc5c5c6c(ccc54)oc4ccccc46)c(-c4nc(-c5ccccc5)nc(-c5ccccc5)n4)n3)c2)cc1 ; c1ccc(-c2cnc(-n3c4ccccc4c4cc5c(cc43)oc3ccccc35)c(-c3nc(-c4ccccc4)nc(-c4ccccc4)n3)c2)cc1 ; c1ccc(-c2cnc(-n3c4ccccc4c4ccc5c6ccccc6oc5c43)c(-c3nc(-c4ccccc4)nc(-c4ccccc4)n3)c2)cc1]

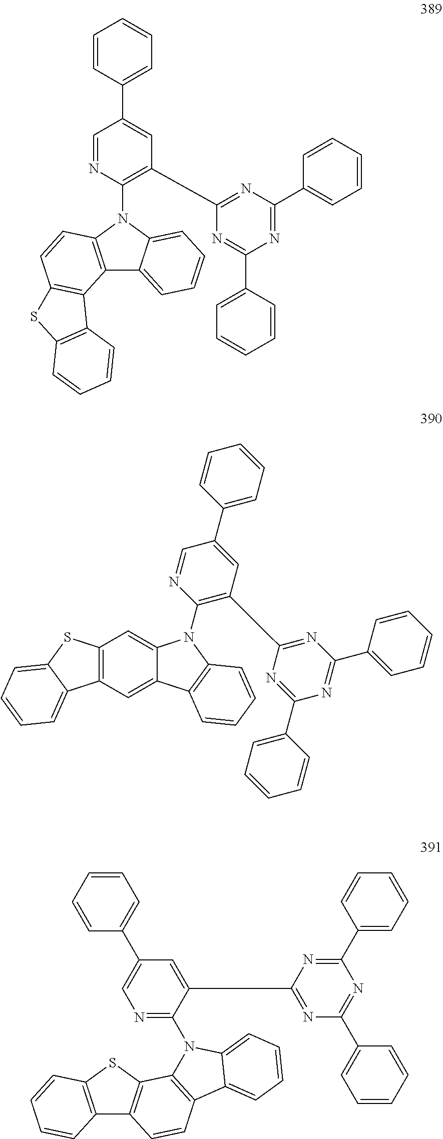 [SMILES: c1ccc(-c2cnc(-n3c4ccccc4c4c5c(ccc43)sc3ccccc35)c(-c3nc(-c4ccccc4)nc(-c4ccccc4)n3)c2)cc1 ; c1ccc(-c2cnc(-n3c4ccccc4c4cc5c(cc43)sc3ccccc35)c(-c3nc(-c4ccccc4)nc(-c4ccccc4)n3)c2)cc1 ; c1ccc(-c2cnc(-n3c4ccccc4c4ccc5c6ccccc6sc5c43)c(-c3nc(-c4ccccc4)nc(-c4ccccc4)n3)c2)cc1]

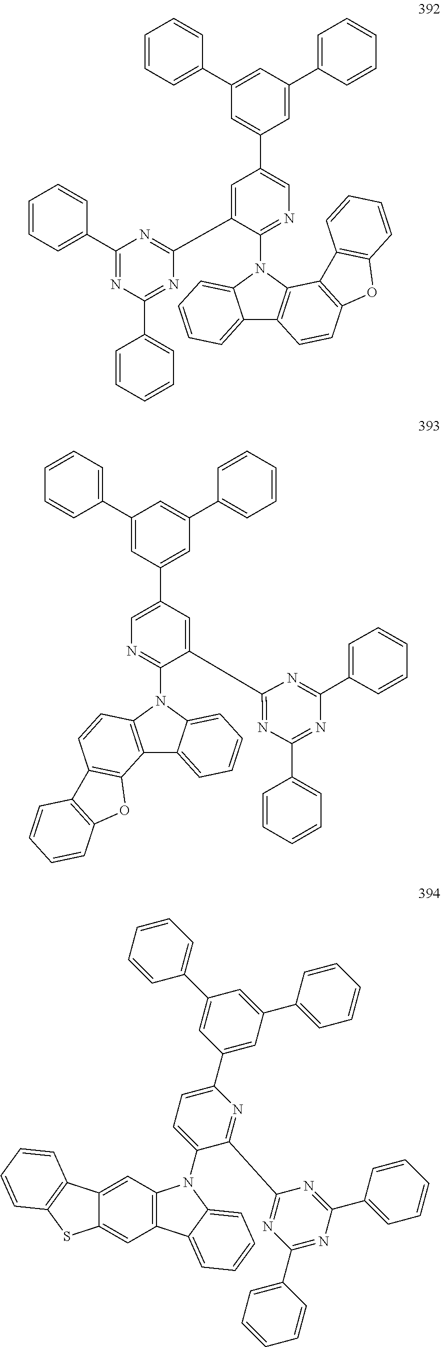 [SMILES: c1ccc(-c2cc(-c3ccccc3)cc(-c3cnc(-n4c5ccccc5c5ccc6oc7ccccc7c6c54)c(-c4nc(-c5ccccc5)nc(-c5ccccc5)n4)c3)c2)cc1 ; c1ccc(-c2cc(-c3ccccc3)cc(-c3cnc(-n4c5ccccc5c5c6oc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6sc7ccccc7c6cc54)c(-c4nc(-c5ccccc5)nc(-c5ccccc5)n4)n3)c2)cc1]

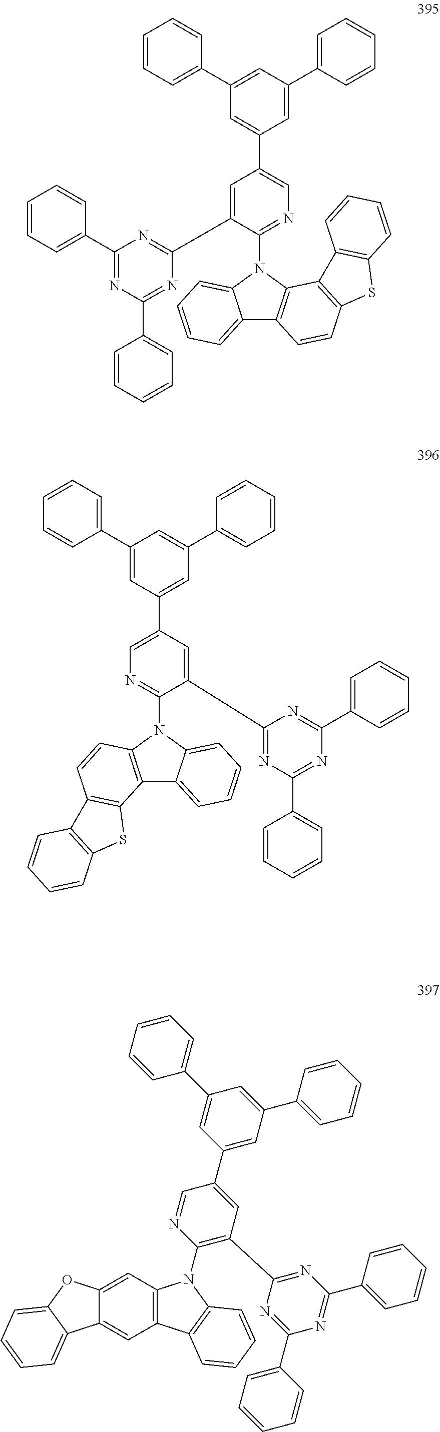 [SMILES: c1ccc(-c2cc(-c3ccccc3)cc(-c3cnc(-n4c5ccccc5c5ccc6sc7ccccc7c6c54)c(-c4nc(-c5ccccc5)nc(-c5ccccc5)n4)c3)c2)cc1 ; c1ccc(-c2cc(-c3ccccc3)cc(-c3cnc(-n4c5ccccc5c5c6sc7ccccc7c6ccc54)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6c(cc54)oc4ccccc46)c(-c4nc(-c5ccccc5)nc(-c5ccccc5)n4)c3)c2)cc1]

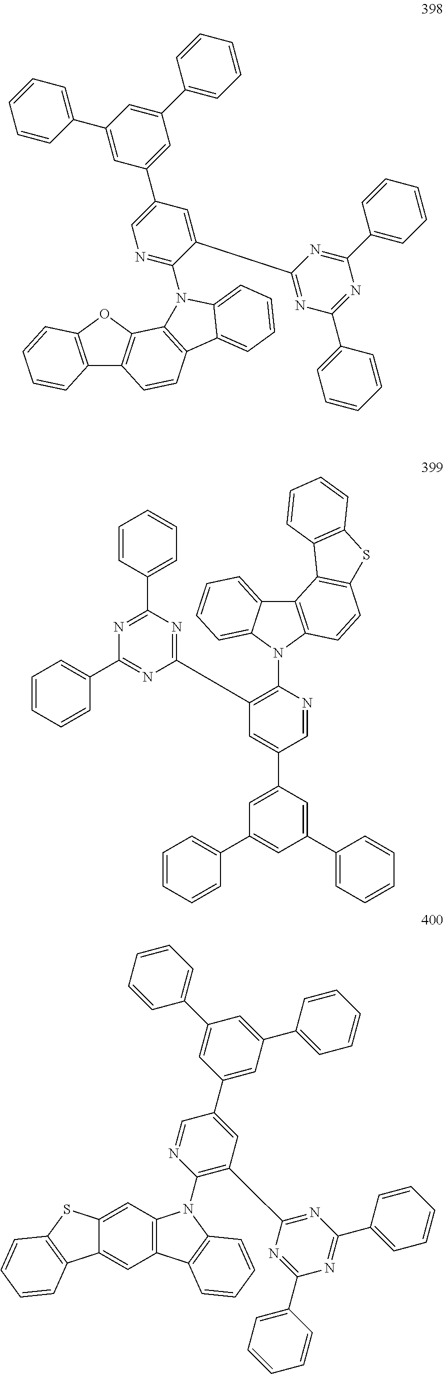 [SMILES: c1ccc(-c2cc(-c3ccccc3)cc(-c3cnc(-n4c5ccccc5c5ccc6c7ccccc7oc6c54)c(-c4nc(-c5ccccc5)nc(-c5ccccc5)n4)c3)c2)cc1 ; c1ccc(-c2cc(-c3ccccc3)cc(-c3cnc(-n4c5ccccc5c5c6c(ccc54)sc4ccccc46)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6c(cc54)sc4ccccc46)c(-c4nc(-c5ccccc5)nc(-c5ccccc5)n4)c3)c2)cc1]

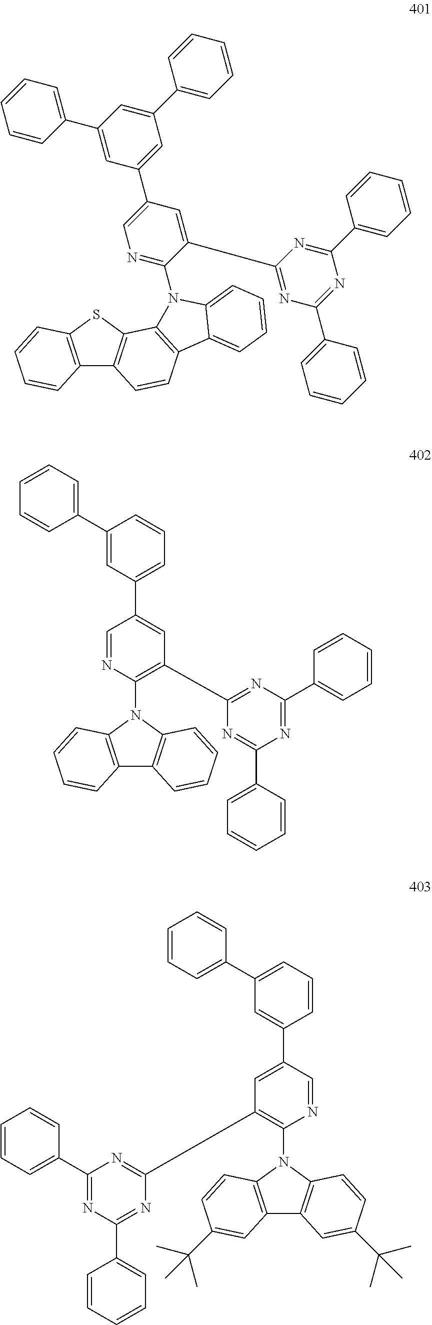 [SMILES: c1ccc(-c2cc(-c3ccccc3)cc(-c3cnc(-n4c5ccccc5c5ccc6c7ccccc7sc6c54)c(-c4nc(-c5ccccc5)nc(-c5ccccc5)n4)c3)c2)cc1 ; c1ccc(-c2cccc(-c3cnc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ncc(-c2cccc(-c3ccccc3)c2)cc1-c1nc(-c2ccccc2)nc(-c2ccccc2)n1]

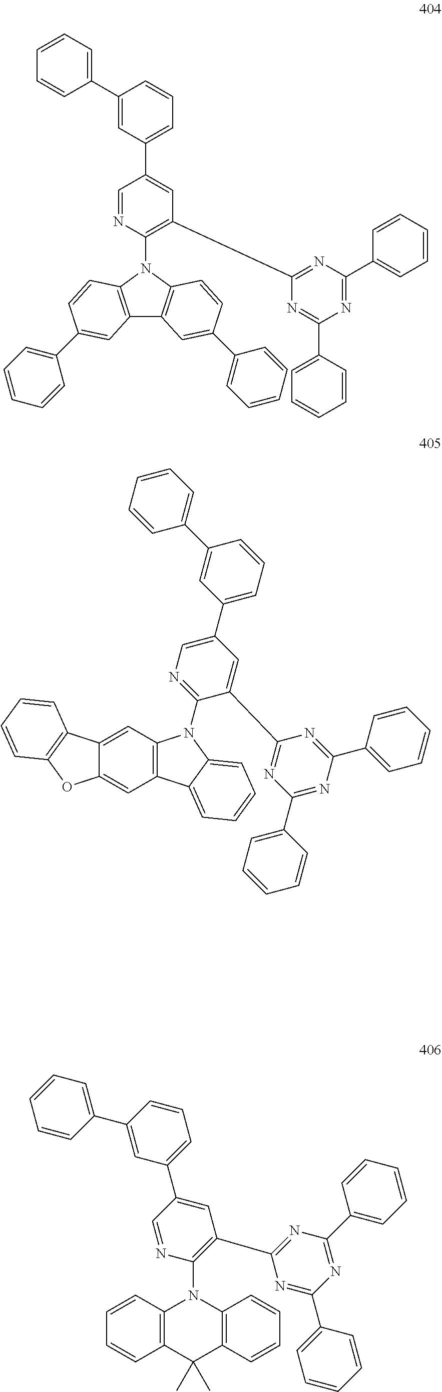 [SMILES: c1ccc(-c2cccc(-c3cnc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2cccc(-c3cnc(-n4c5ccccc5c5cc6oc7ccccc7c6cc54)c(-c4nc(-c5ccccc5)nc(-c5ccccc5)n4)c3)c2)cc1 ; CC1(C)c2ccccc2N(c2ncc(-c3cccc(-c4ccccc4)c3)cc2-c2nc(-c3ccccc3)nc(-c3ccccc3)n2)c2ccccc21]

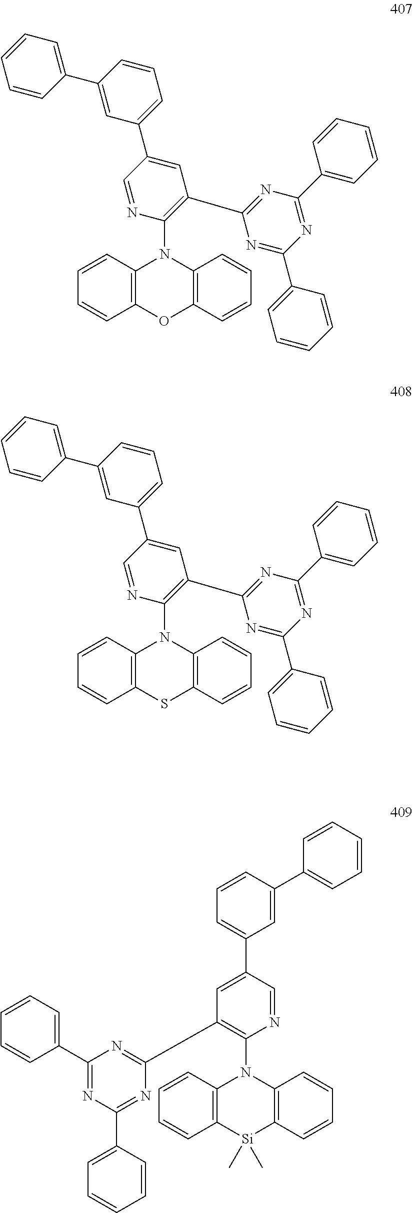 [SMILES: c1ccc(-c2cccc(-c3cnc(N4c5ccccc5Oc5ccccc54)c(-c4nc(-c5ccccc5)nc(-c5ccccc5)n4)c3)c2)cc1 ; c1ccc(-c2cccc(-c3cnc(N4c5ccccc5Sc5ccccc54)c(-c4nc(-c5ccccc5)nc(-c5ccccc5)n4)c3)c2)cc1 ; C[Si]1(C)c2ccccc2N(c2ncc(-c3cccc(-c4ccccc4)c3)cc2-c2nc(-c3ccccc3)nc(-c3ccccc3)n2)c2ccccc21]

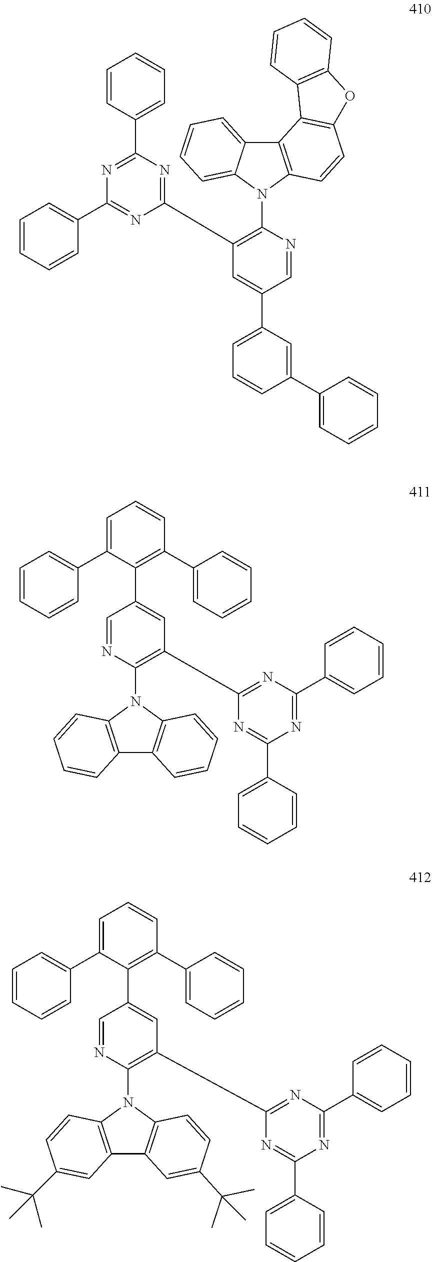 [SMILES: c1ccc(-c2cccc(-c3cnc(-n4c5ccccc5c5c6c(ccc54)oc4ccccc46)c(-c4nc(-c5ccccc5)nc(-c5ccccc5)n4)c3)c2)cc1 ; c1ccc(-c2nc(-c3ccccc3)nc(-c3cc(-c4c(-c5ccccc5)cccc4-c4ccccc4)cnc3-n3c4ccccc4c4ccccc43)n2)cc1 ; CC(C)(C)c1ccc2c(c1)c1cc(C(C)(C)C)ccc1n2-c1ncc(-c2c(-c3ccccc3)cccc2-c2ccccc2)cc1-c1nc(-c2ccccc2)nc(-c2ccccc2)n1]

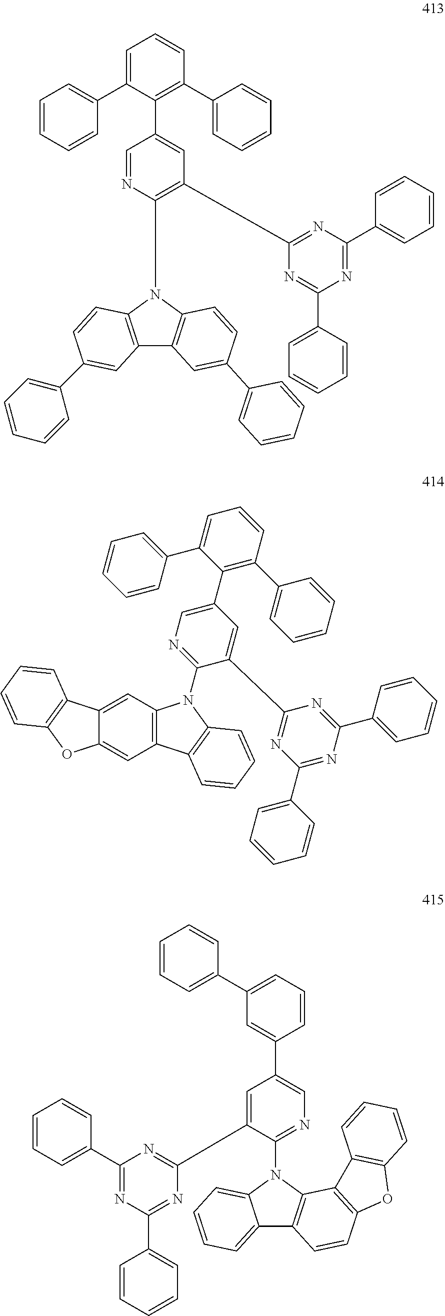 [SMILES: c1ccc(-c2ccc3c(c2)c2cc(-c4ccccc4)ccc2n3-c2ncc(-c3c(-c4ccccc4)cccc3-c3ccccc3)cc2-c2nc(-c3ccccc3)nc(-c3ccccc3)n2)cc1 ; c1ccc(-c2nc(-c3ccccc3)nc(-c3cc(-c4c(-c5ccccc5)cccc4-c4ccccc4)cnc3-n3c4ccccc4c4cc5oc6ccccc6c5cc43)n2)cc1 ; c1ccc(-c2cccc(-c3cnc(-n4c5ccccc5c5ccc6oc7ccccc7c6c54)c(-c4nc(-c5ccccc5)nc(-c5ccccc5)n4)c3)c2)cc1]

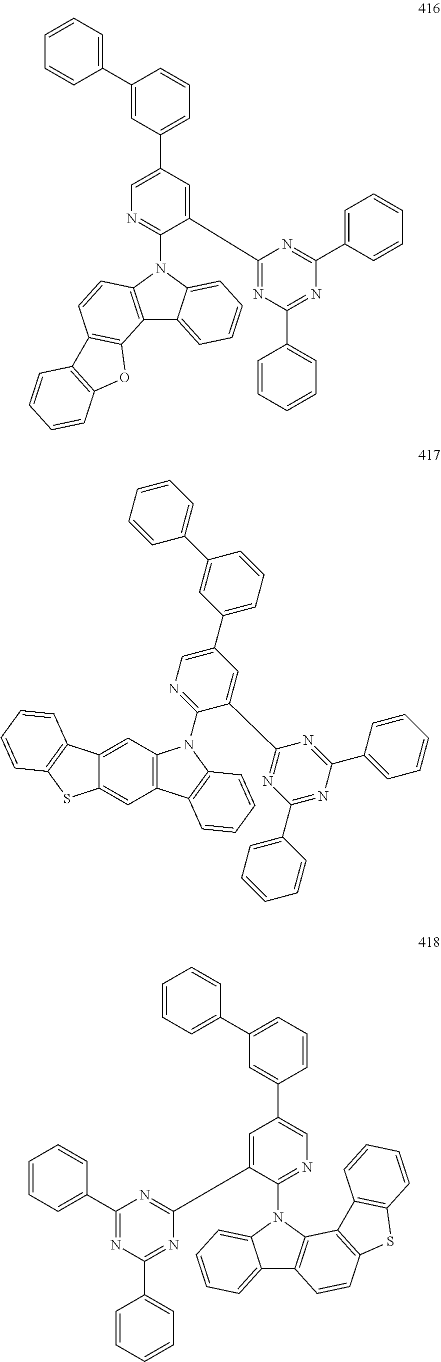 [SMILES: c1ccc(-c2cccc(-c3cnc(-n4c5ccccc5c5c6oc7ccccc7c6ccc54)c(-c4nc(-c5ccccc5)nc(-c5ccccc5)n4)c3)c2)cc1 ; c1ccc(-c2cccc(-c3cnc(-n4c5ccccc5c5cc6sc7ccccc7c6cc54)c(-c4nc(-c5ccccc5)nc(-c5ccccc5)n4)c3)c2)cc1 ; c1ccc(-c2cccc(-c3cnc(-n4c5ccccc5c5ccc6sc7ccccc7c6c54)c(-c4nc(-c5ccccc5)nc(-c5ccccc5)n4)c3)c2)cc1]

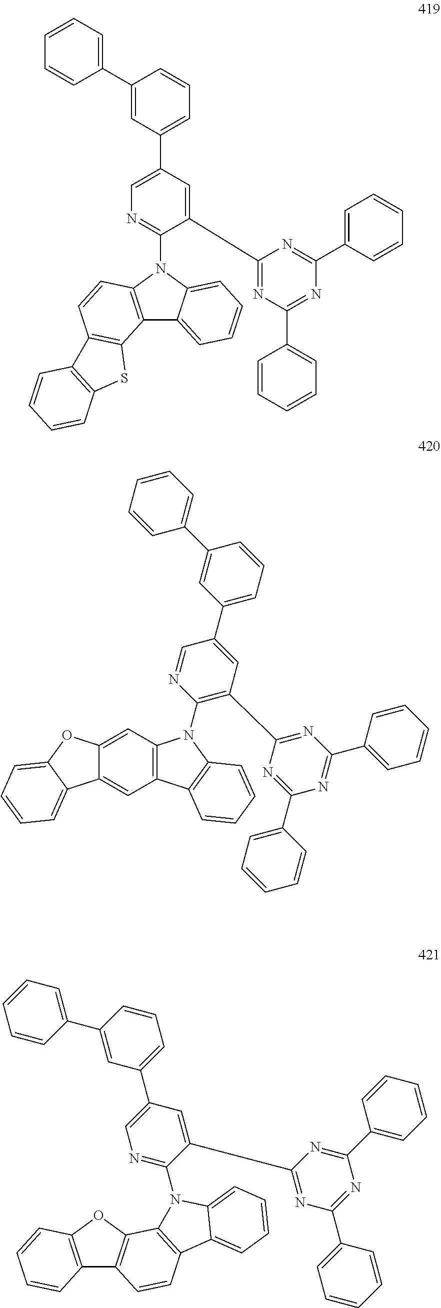 [SMILES: c1ccc(-c2cccc(-c3cnc(-n4c5ccccc5c5c6sc7ccccc7c6ccc54)c(-c4nc(-c5ccccc5)nc(-c5ccccc5)n4)c3)c2)cc1 ; c1ccc(-c2cccc(-c3cnc(-n4c5ccccc5c5cc6c(cc54)oc4ccccc46)c(-c4nc(-c5ccccc5)nc(-c5ccccc5)n4)c3)c2)cc1 ; c1ccc(-c2cccc(-c3cnc(-n4c5ccccc5c5ccc6c7ccccc7oc6c54)c(-c4nc(-c5ccccc5)nc(-c5ccccc5)n4)c3)c2)cc1]

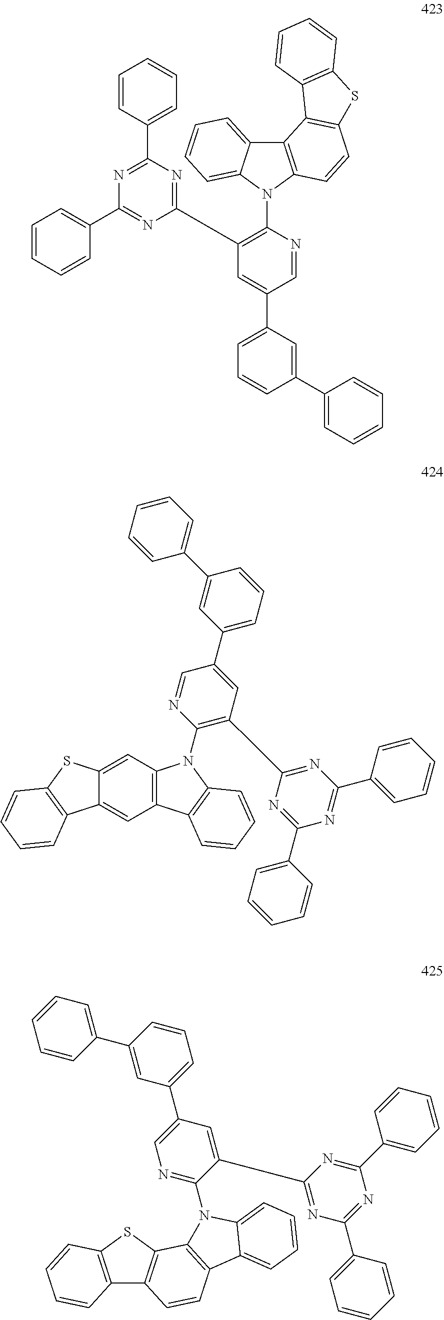 [SMILES: c1ccc(-c2cccc(-c3cnc(-n4c5ccccc5c5c6c(ccc54)sc4ccccc46)c(-c4nc(-c5ccccc5)nc(-c5ccccc5)n4)c3)c2)cc1 ; c1ccc(-c2cccc(-c3cnc(-n4c5ccccc5c5cc6c(cc54)sc4ccccc46)c(-c4nc(-c5ccccc5)nc(-c5ccccc5)n4)c3)c2)cc1 ; c1ccc(-c2cccc(-c3cnc(-n4c5ccccc5c5ccc6c7ccccc7sc6c54)c(-c4nc(-c5ccccc5)nc(-c5ccccc5)n4)c3)c2)cc1]

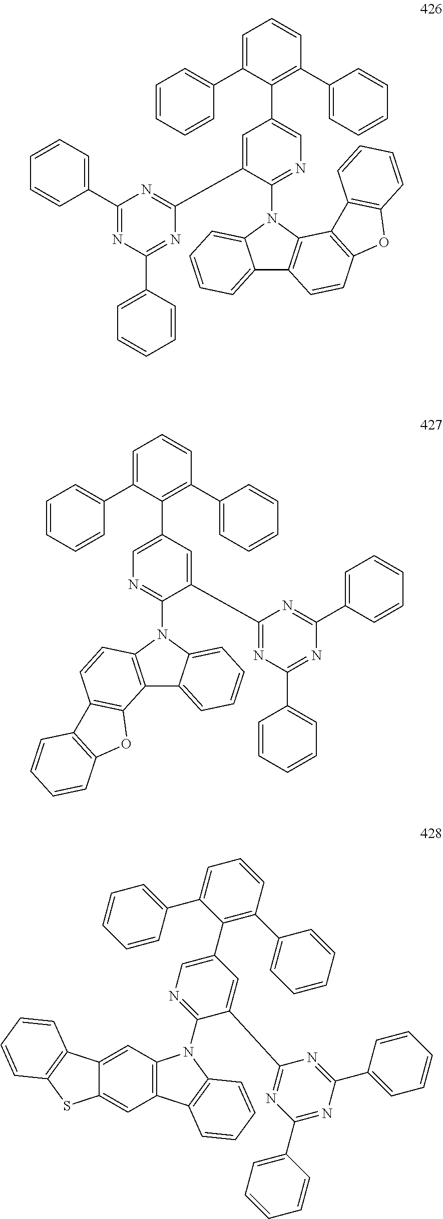 [SMILES: c1ccc(-c2nc(-c3ccccc3)nc(-c3cc(-c4c(-c5ccccc5)cccc4-c4ccccc4)cnc3-n3c4ccccc4c4ccc5oc6ccccc6c5c43)n2)cc1 ; c1ccc(-c2nc(-c3ccccc3)nc(-c3cc(-c4c(-c5ccccc5)cccc4-c4ccccc4)cnc3-n3c4ccccc4c4c5oc6ccccc6c5ccc43)n2)cc1 ; c1ccc(-c2nc(-c3ccccc3)nc(-c3cc(-c4c(-c5ccccc5)cccc4-c4ccccc4)cnc3-n3c4ccccc4c4cc5sc6ccccc6c5cc43)n2)cc1]

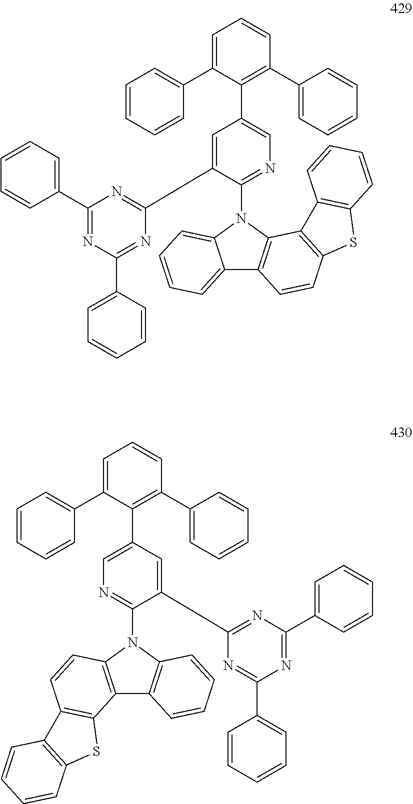 [SMILES: c1ccc(-c2nc(-c3ccccc3)nc(-c3cc(-c4c(-c5ccccc5)cccc4-c4ccccc4)cnc3-n3c4ccccc4c4ccc5sc6ccccc6c5c43)n2)cc1 ; c1ccc(-c2nc(-c3ccccc3)nc(-c3cc(-c4c(-c5ccccc5)cccc4-c4ccccc4)cnc3-n3c4ccccc4c4c5sc6ccccc6c5ccc43)n2)cc1]

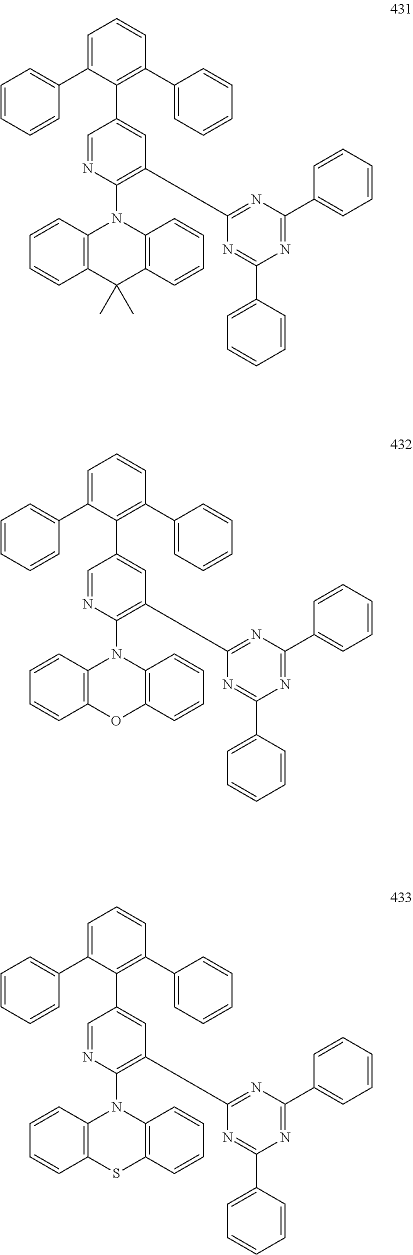 [SMILES: CC1(C)c2ccccc2N(c2ncc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)cnc3N3c4ccccc4Oc4ccccc43)n2)cc1 ; c1ccc(-c2nc(-c3ccccc3)nc(-c3cc(-c4c(-c5ccccc5)cccc4-c4ccccc4)cnc3N3c4ccccc4Sc4ccccc43)n2)cc1]

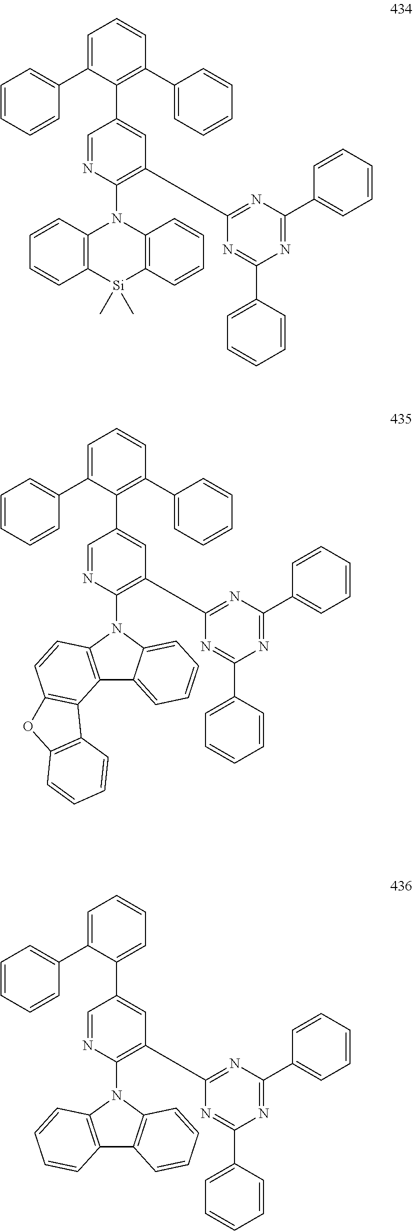 [SMILES: C[Si]1(C)c2ccccc2N(c2ncc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)cnc3-n3c4ccccc4c4c5c(ccc43)oc3ccccc35)n2)cc1 ; c1ccc(-c2nc(-c3ccccc3)nc(-c3cc(-c4ccccc4-c4ccccc4)cnc3-n3c4ccccc4c4ccccc43)n2)cc1]

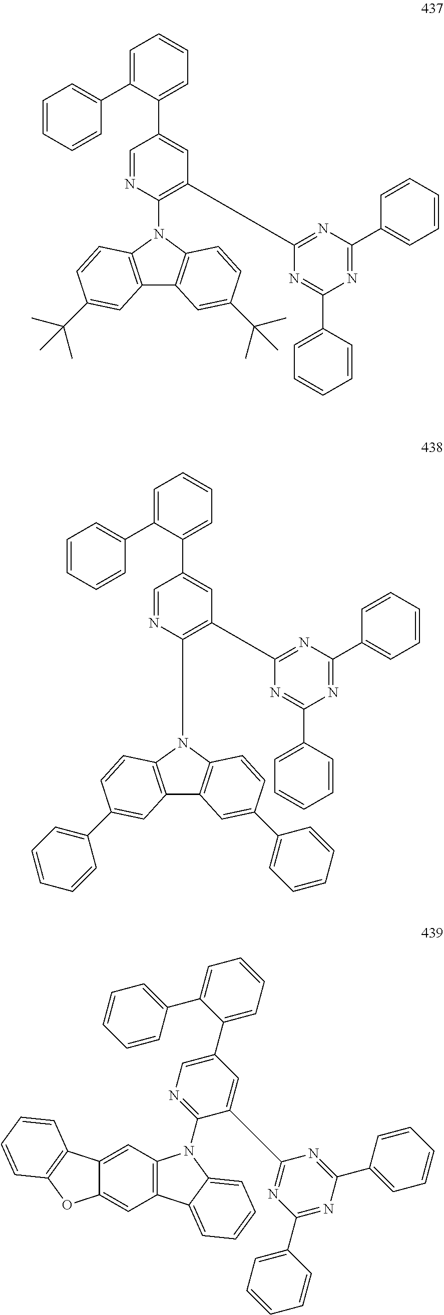 [SMILES: CC(C)(C)c1ccc2c(c1)c1cc(C(C)(C)C)ccc1n2-c1ncc(-c2ccccc2-c2ccccc2)cc1-c1nc(-c2ccccc2)nc(-c2ccccc2)n1 ; c1ccc(-c2ccc3c(c2)c2cc(-c4ccccc4)ccc2n3-c2ncc(-c3ccccc3-c3ccccc3)cc2-c2nc(-c3ccccc3)nc(-c3ccccc3)n2)cc1 ; c1ccc(-c2nc(-c3ccccc3)nc(-c3cc(-c4ccccc4-c4ccccc4)cnc3-n3c4ccccc4c4cc5oc6ccccc6c5cc43)n2)cc1]

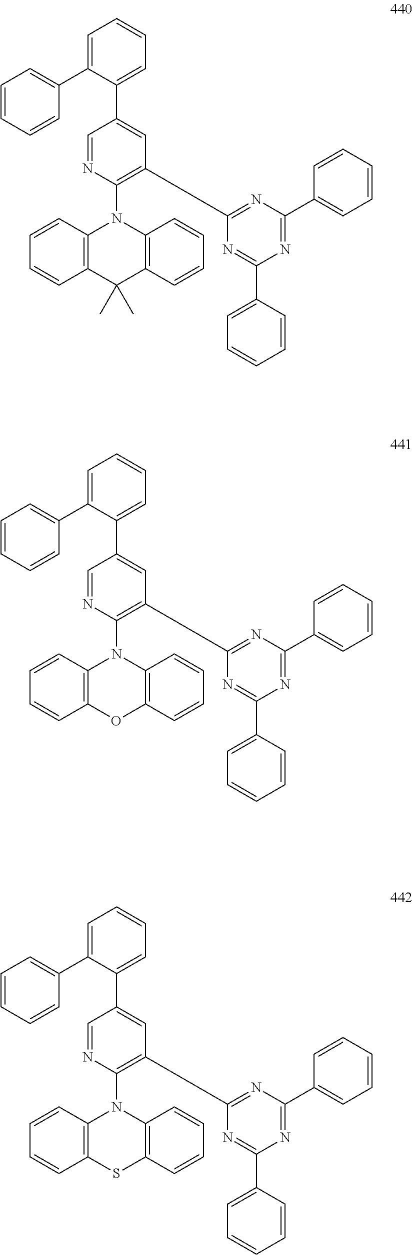 [SMILES: CC1(C)c2ccccc2N(c2ncc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)cnc3N3c4ccccc4Oc4ccccc43)n2)cc1 ; c1ccc(-c2nc(-c3ccccc3)nc(-c3cc(-c4ccccc4-c4ccccc4)cnc3N3c4ccccc4Sc4ccccc43)n2)cc1]

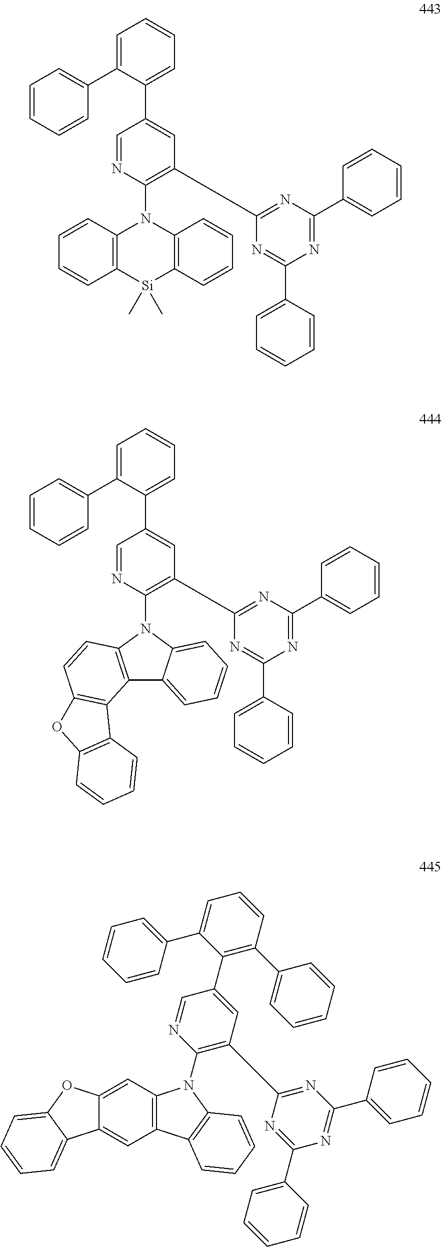 [SMILES: C[Si]1(C)c2ccccc2N(c2ncc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)cnc3-n3c4ccccc4c4c5c(ccc43)oc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4cc5c(cc43)oc3ccccc35)n2)cc1]

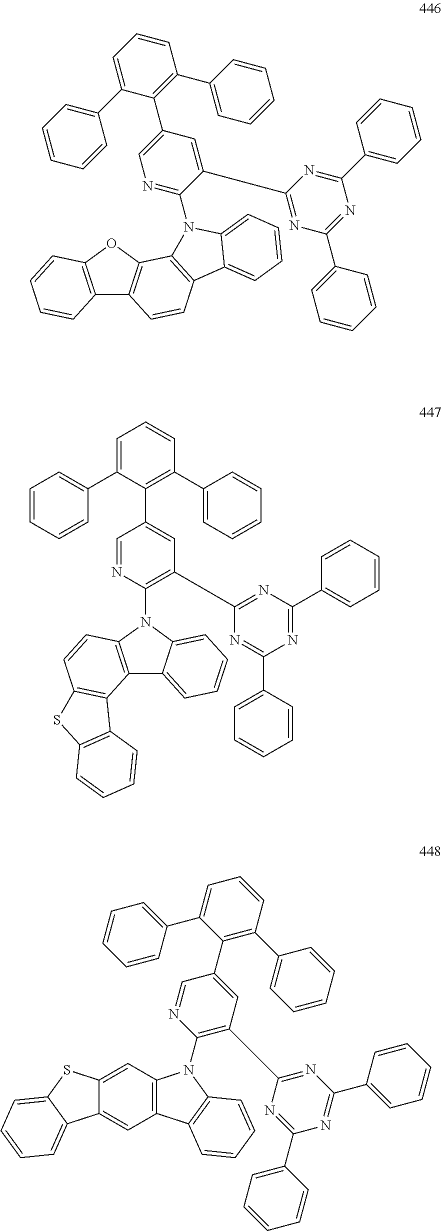 [SMILES: c1ccc(-c2nc(-c3ccccc3)nc(-c3cc(-c4c(-c5ccccc5)cccc4-c4ccccc4)cnc3-n3c4ccccc4c4ccc5c6ccccc6oc5c43)n2)cc1 ; c1ccc(-c2nc(-c3ccccc3)nc(-c3cc(-c4c(-c5ccccc5)cccc4-c4ccccc4)cnc3-n3c4ccccc4c4c5c(ccc43)sc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4cc5c(cc43)sc3ccccc35)n2)cc1]

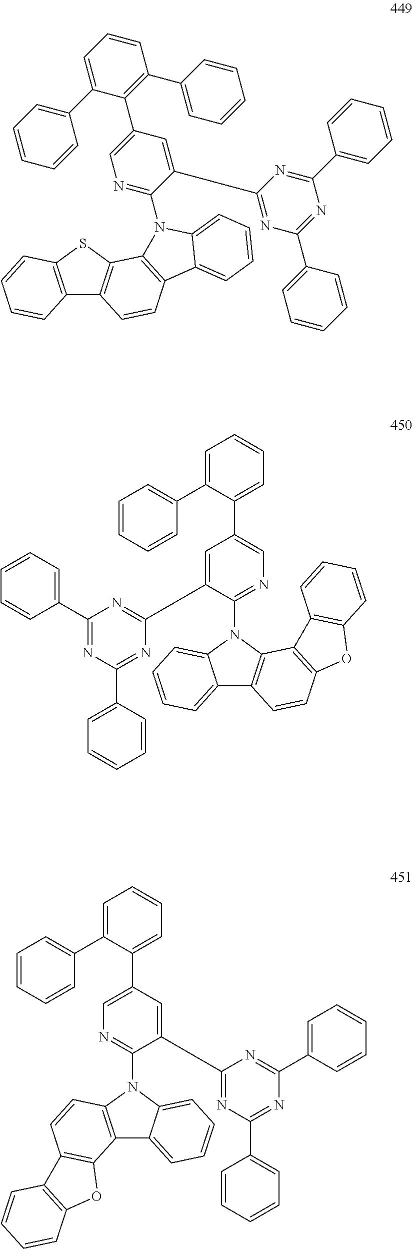 [SMILES: c1ccc(-c2nc(-c3ccccc3)nc(-c3cc(-c4c(-c5ccccc5)cccc4-c4ccccc4)cnc3-n3c4ccccc4c4ccc5c6ccccc6sc5c43)n2)cc1 ; c1ccc(-c2nc(-c3ccccc3)nc(-c3cc(-c4ccccc4-c4ccccc4)cnc3-n3c4ccccc4c4ccc5oc6ccccc6c5c43)n2)cc1 ; c1ccc(-c2nc(-c3ccccc3)nc(-c3cc(-c4ccccc4-c4ccccc4)cnc3-n3c4ccccc4c4c5oc6ccccc6c5ccc43)n2)cc1]

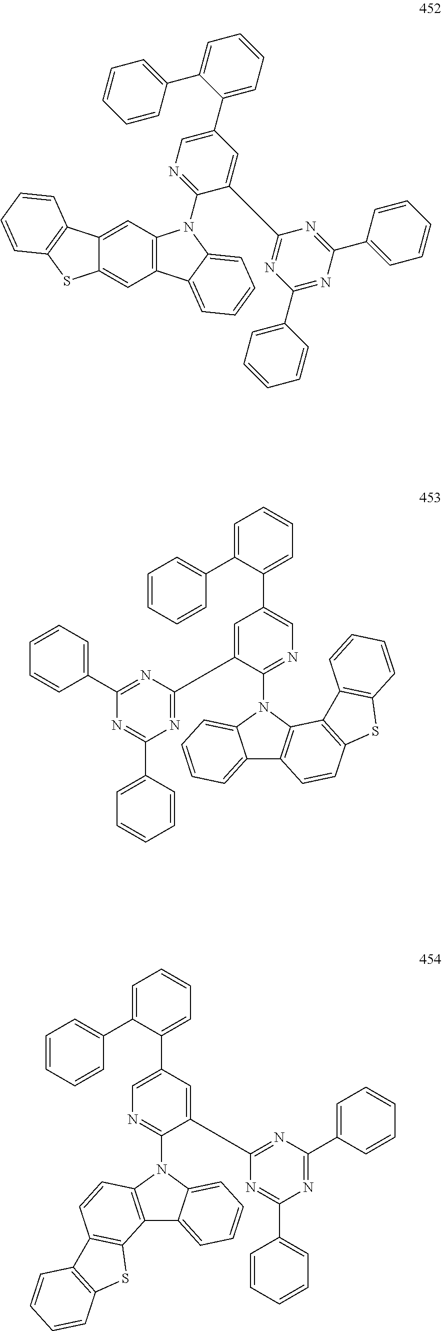 [SMILES: c1ccc(-c2nc(-c3ccccc3)nc(-c3cc(-c4ccccc4-c4ccccc4)cnc3-n3c4ccccc4c4cc5sc6ccccc6c5cc43)n2)cc1 ; c1ccc(-c2nc(-c3ccccc3)nc(-c3cc(-c4ccccc4-c4ccccc4)cnc3-n3c4ccccc4c4ccc5sc6ccccc6c5c43)n2)cc1 ; c1ccc(-c2nc(-c3ccccc3)nc(-c3cc(-c4ccccc4-c4ccccc4)cnc3-n3c4ccccc4c4c5sc6ccccc6c5ccc43)n2)cc1]

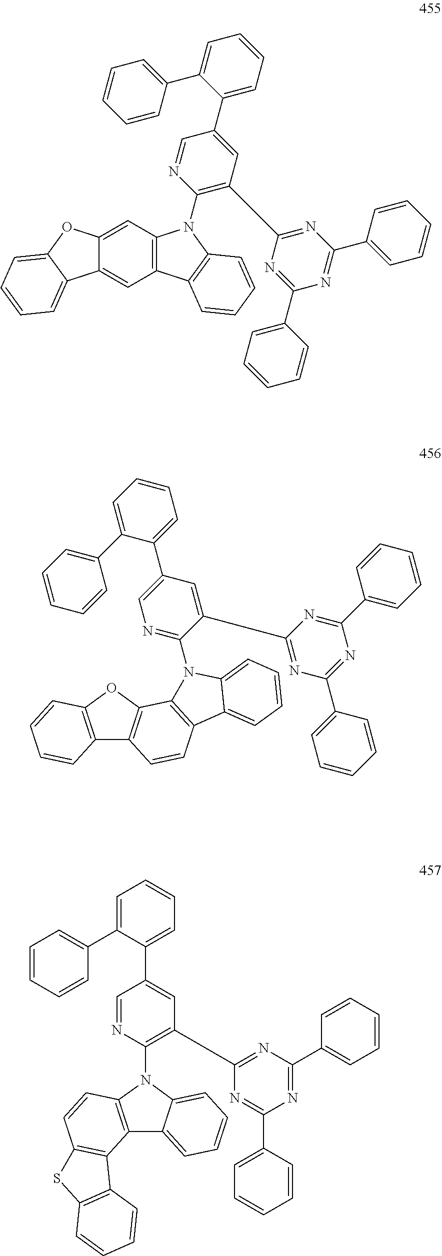 [SMILES: c1ccc(-c2nc(-c3ccccc3)nc(-c3cc(-c4ccccc4-c4ccccc4)cnc3-n3c4ccccc4c4cc5c(cc43)oc3ccccc35)n2)cc1 ; c1ccc(-c2nc(-c3ccccc3)nc(-c3cc(-c4ccccc4-c4ccccc4)cnc3-n3c4ccccc4c4ccc5c6ccccc6oc5c43)n2)cc1 ; c1ccc(-c2nc(-c3ccccc3)nc(-c3cc(-c4ccccc4-c4ccccc4)cnc3-n3c4ccccc4c4c5c(ccc43)sc3ccccc35)n2)cc1]

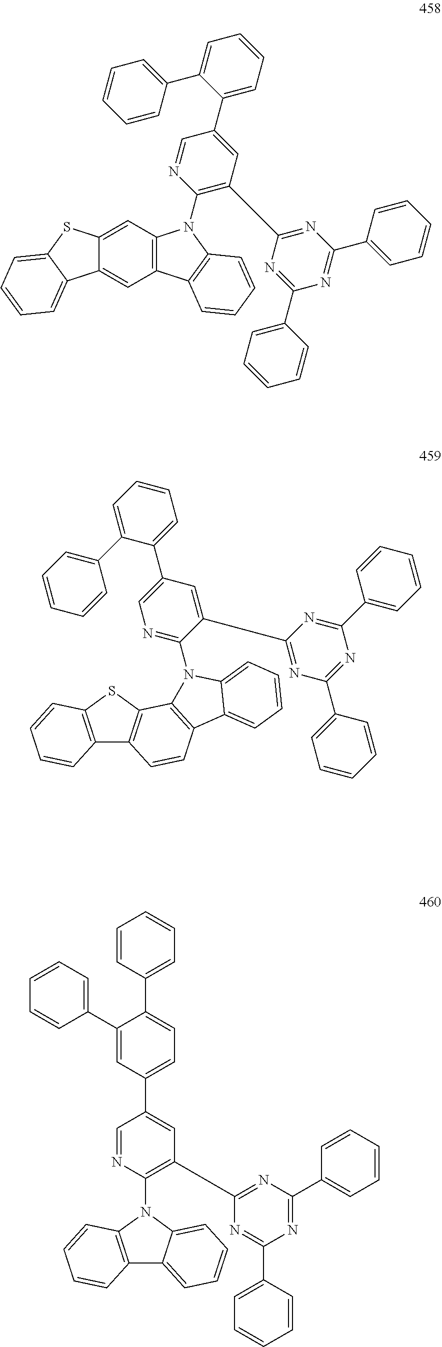 [SMILES: c1ccc(-c2nc(-c3ccccc3)nc(-c3cc(-c4ccccc4-c4ccccc4)cnc3-n3c4ccccc4c4cc5c(cc43)sc3ccccc35)n2)cc1 ; c1ccc(-c2nc(-c3ccccc3)nc(-c3cc(-c4ccccc4-c4ccccc4)cnc3-n3c4ccccc4c4ccc5c6ccccc6sc5c43)n2)cc1 ; c1ccc(-c2nc(-c3ccccc3)nc(-c3cc(-c4ccc(-c5ccccc5)c(-c5ccccc5)c4)cnc3-n3c4ccccc4c4ccccc43)n2)cc1]

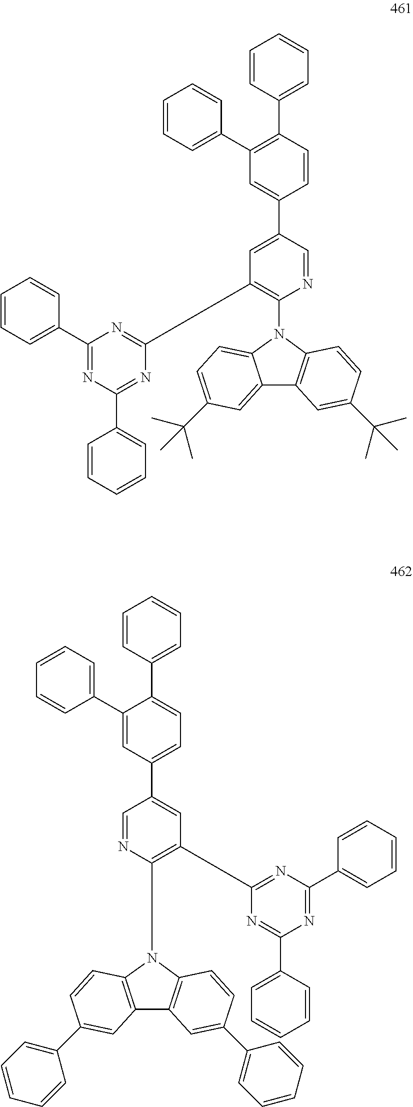 [SMILES: CC(C)(C)c1ccc2c(c1)c1cc(C(C)(C)C)ccc1n2-c1ncc(-c2ccc(-c3ccccc3)c(-c3ccccc3)c2)cc1-c1nc(-c2ccccc2)nc(-c2ccccc2)n1 ; c1ccc(-c2ccc3c(c2)c2cc(-c4ccccc4)ccc2n3-c2ncc(-c3ccc(-c4ccccc4)c(-c4ccccc4)c3)cc2-c2nc(-c3ccccc3)nc(-c3ccccc3)n2)cc1]

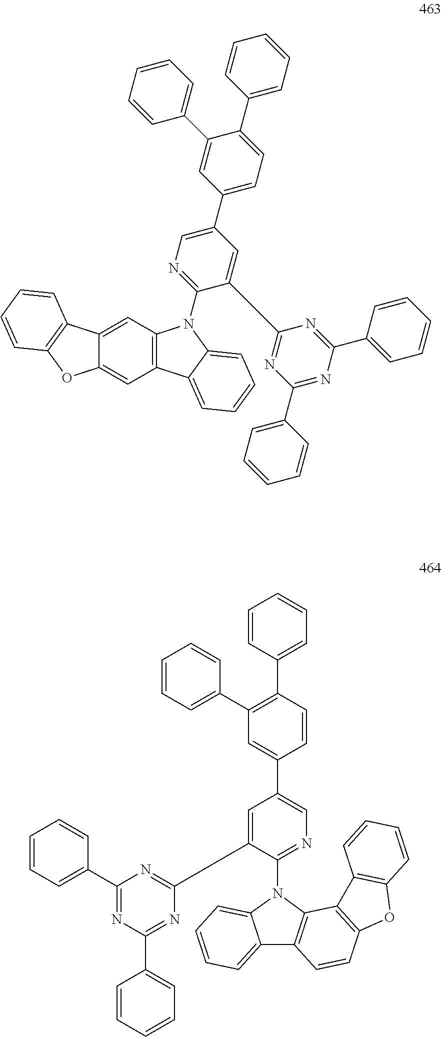 [SMILES: c1ccc(-c2nc(-c3ccccc3)nc(-c3cc(-c4ccc(-c5ccccc5)c(-c5ccccc5)c4)cnc3-n3c4ccccc4c4cc5oc6ccccc6c5cc43)n2)cc1 ; c1ccc(-c2nc(-c3ccccc3)nc(-c3cc(-c4ccc(-c5ccccc5)c(-c5ccccc5)c4)cnc3-n3c4ccccc4c4ccc5oc6ccccc6c5c43)n2)cc1]

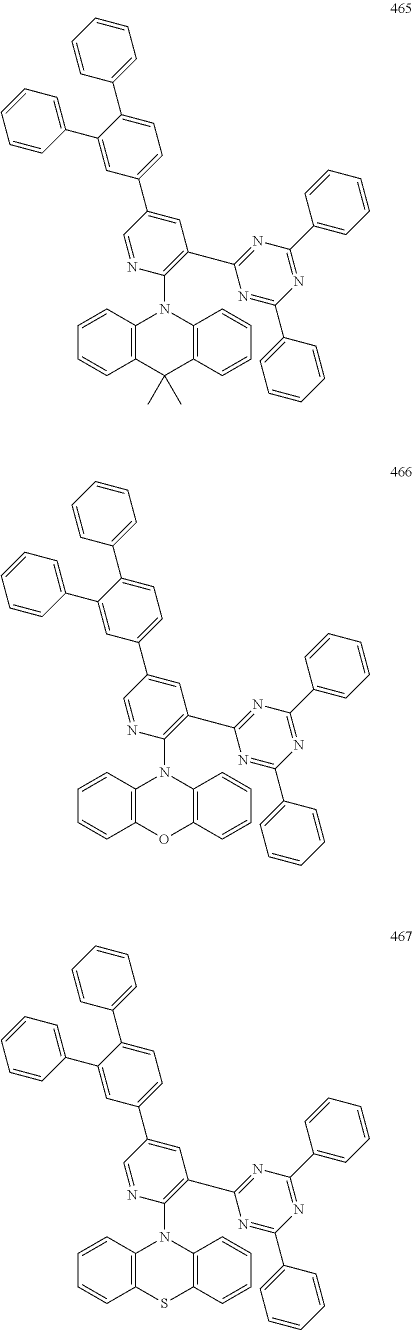 [SMILES: CC1(C)c2ccccc2N(c2ncc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3cc(-c4ccc(-c5ccccc5)c(-c5ccccc5)c4)cnc3N3c4ccccc4Oc4ccccc43)n2)cc1 ; c1ccc(-c2nc(-c3ccccc3)nc(-c3cc(-c4ccc(-c5ccccc5)c(-c5ccccc5)c4)cnc3N3c4ccccc4Sc4ccccc43)n2)cc1]

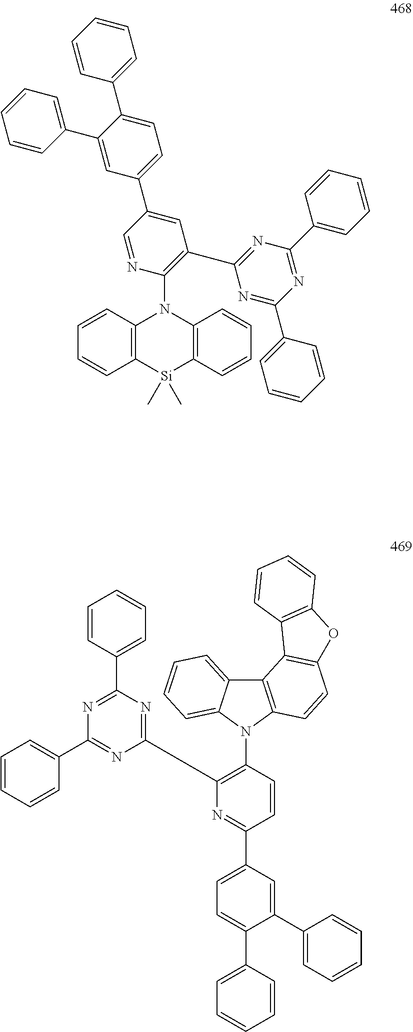 [SMILES: C[Si]1(C)c2ccccc2N(c2ncc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3nc(-c4ccc(-c5ccccc5)c(-c5ccccc5)c4)ccc3-n3c4ccccc4c4c5c(ccc43)oc3ccccc35)n2)cc1]

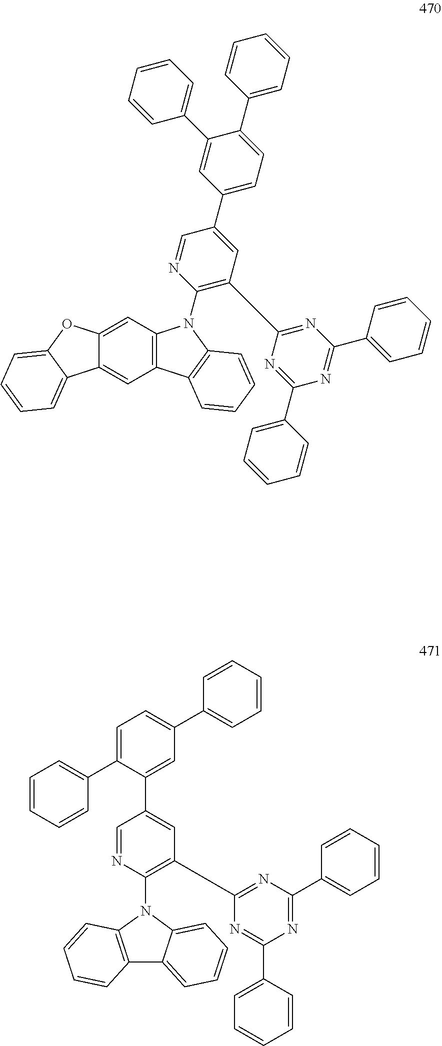 [SMILES: c1ccc(-c2nc(-c3ccccc3)nc(-c3cc(-c4ccc(-c5ccccc5)c(-c5ccccc5)c4)cnc3-n3c4ccccc4c4cc5c(cc43)oc3ccccc35)n2)cc1 ; c1ccc(-c2ccc(-c3ccccc3)c(-c3cnc(-n4c5ccccc5c5ccccc54)c(-c4nc(-c5ccccc5)nc(-c5ccccc5)n4)c3)c2)cc1]

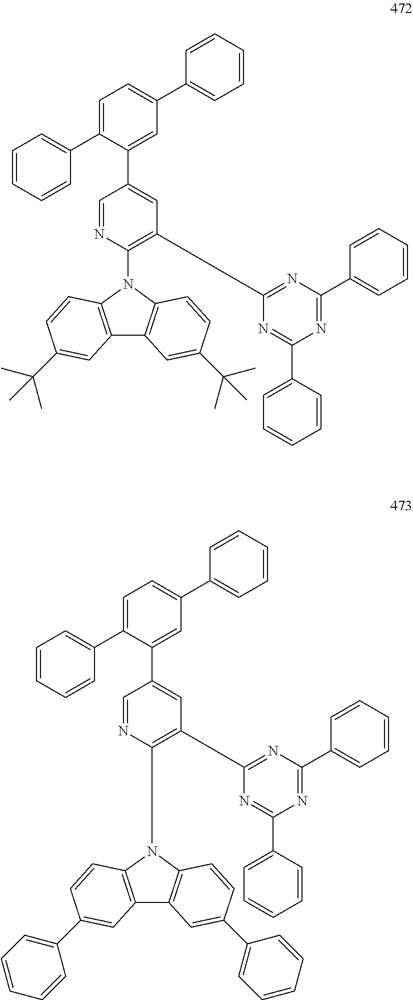 [SMILES: CC(C)(C)c1ccc2c(c1)c1cc(C(C)(C)C)ccc1n2-c1ncc(-c2cc(-c3ccccc3)ccc2-c2ccccc2)cc1-c1nc(-c2ccccc2)nc(-c2ccccc2)n1 ; c1ccc(-c2ccc(-c3ccccc3)c(-c3cnc(-n4c5ccc(-c6ccccc6)cc5c5cc(-c6ccccc6)ccc54)c(-c4nc(-c5ccccc5)nc(-c5ccccc5)n4)c3)c2)cc1]

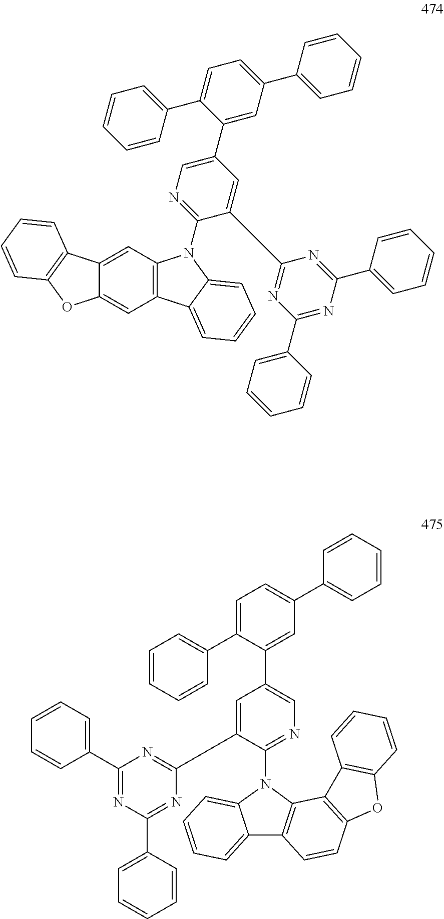 [SMILES: c1ccc(-c2ccc(-c3ccccc3)c(-c3cnc(-n4c5ccccc5c5cc6oc7ccccc7c6cc54)c(-c4nc(-c5ccccc5)nc(-c5ccccc5)n4)c3)c2)cc1 ; c1ccc(-c2ccc(-c3ccccc3)c(-c3cnc(-n4c5ccccc5c5ccc6oc7ccccc7c6c54)c(-c4nc(-c5ccccc5)nc(-c5ccccc5)n4)c3)c2)cc1]

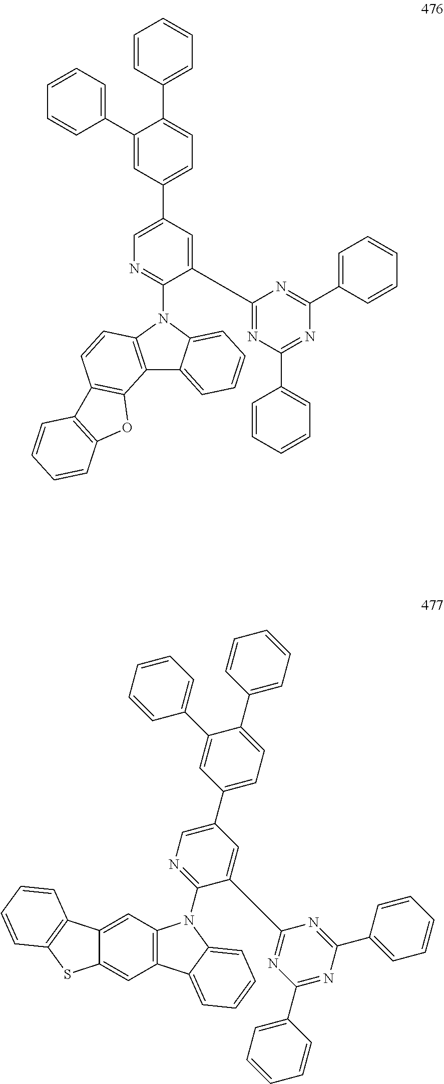 [SMILES: c1ccc(-c2nc(-c3ccccc3)nc(-c3cc(-c4ccc(-c5ccccc5)c(-c5ccccc5)c4)cnc3-n3c4ccccc4c4c5oc6ccccc6c5ccc43)n2)cc1 ; c1ccc(-c2nc(-c3ccccc3)nc(-c3cc(-c4ccc(-c5ccccc5)c(-c5ccccc5)c4)cnc3-n3c4ccccc4c4cc5sc6ccccc6c5cc43)n2)cc1]

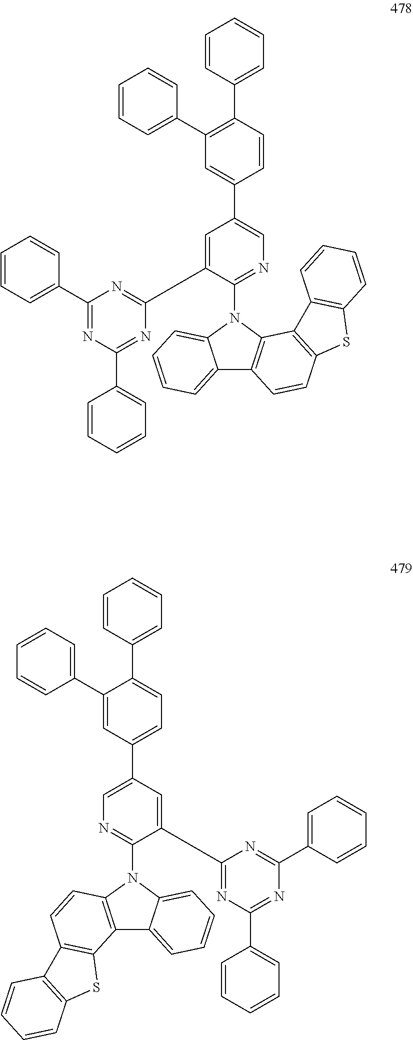 [SMILES: c1ccc(-c2nc(-c3ccccc3)nc(-c3cc(-c4ccc(-c5ccccc5)c(-c5ccccc5)c4)cnc3-n3c4ccccc4c4ccc5sc6ccccc6c5c43)n2)cc1 ; c1ccc(-c2nc(-c3ccccc3)nc(-c3cc(-c4ccc(-c5ccccc5)c(-c5ccccc5)c4)cnc3-n3c4ccccc4c4c5sc6ccccc6c5ccc43)n2)cc1]

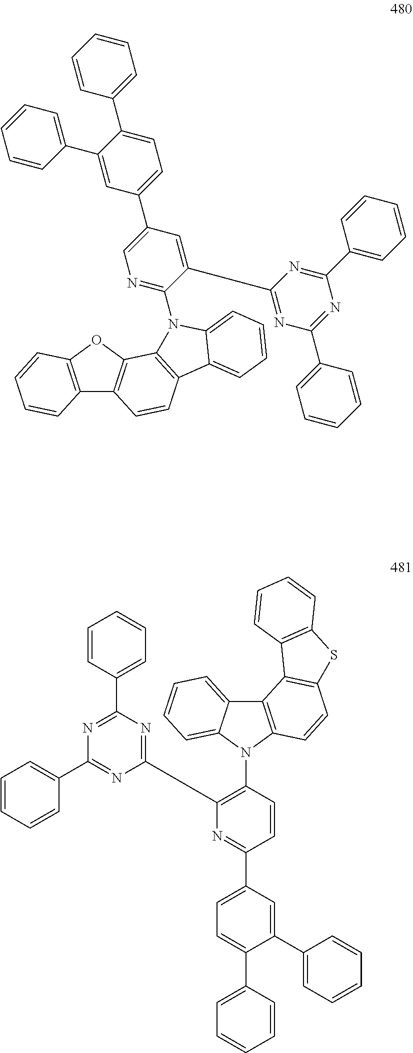 [SMILES: c1ccc(-c2nc(-c3ccccc3)nc(-c3cc(-c4ccc(-c5ccccc5)c(-c5ccccc5)c4)cnc3-n3c4ccccc4c4ccc5c6ccccc6oc5c43)n2)cc1 ; c1ccc(-c2nc(-c3ccccc3)nc(-c3nc(-c4ccc(-c5ccccc5)c(-c5ccccc5)c4)ccc3-n3c4ccccc4c4c5c(ccc43)sc3ccccc35)n2)cc1]

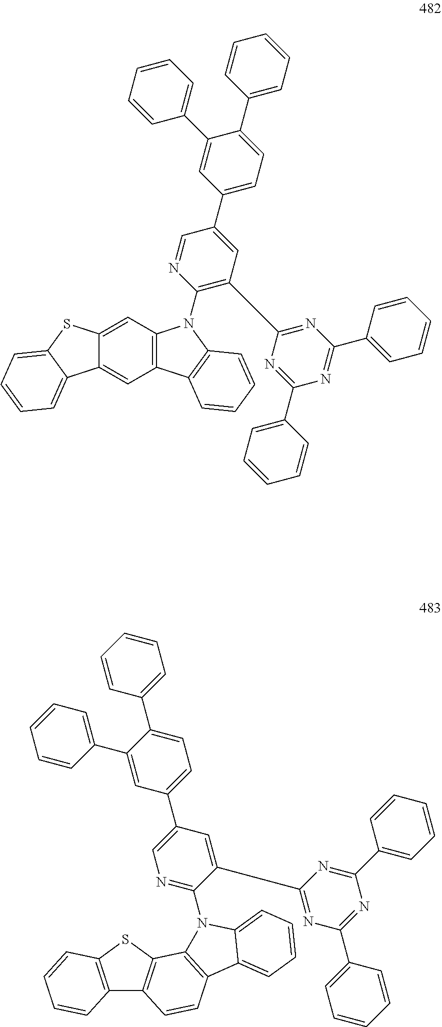 [SMILES: c1ccc(-c2nc(-c3ccccc3)nc(-c3cc(-c4ccc(-c5ccccc5)c(-c5ccccc5)c4)cnc3-n3c4ccccc4c4cc5c(cc43)sc3ccccc35)n2)cc1 ; c1ccc(-c2nc(-c3ccccc3)nc(-c3cc(-c4ccc(-c5ccccc5)c(-c5ccccc5)c4)cnc3-n3c4ccccc4c4ccc5c6ccccc6sc5c43)n2)cc1]

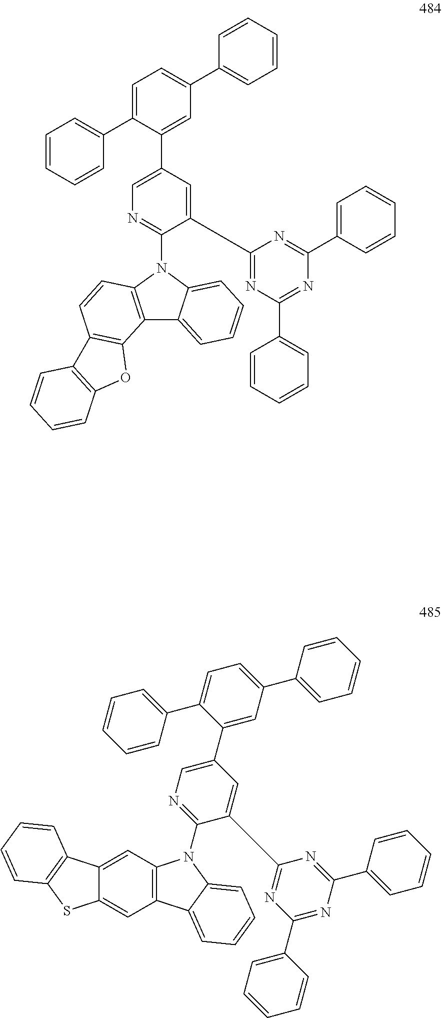 [SMILES: c1ccc(-c2ccc(-c3ccccc3)c(-c3cnc(-n4c5ccccc5c5c6oc7ccccc7c6ccc54)c(-c4nc(-c5ccccc5)nc(-c5ccccc5)n4)c3)c2)cc1 ; c1ccc(-c2ccc(-c3ccccc3)c(-c3cnc(-n4c5ccccc5c5cc6sc7ccccc7c6cc54)c(-c4nc(-c5ccccc5)nc(-c5ccccc5)n4)c3)c2)cc1]

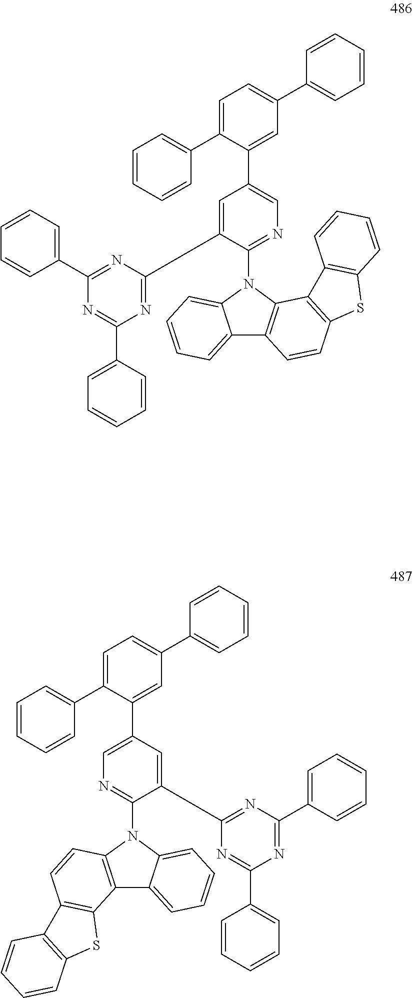 [SMILES: c1ccc(-c2ccc(-c3ccccc3)c(-c3cnc(-n4c5ccccc5c5ccc6sc7ccccc7c6c54)c(-c4nc(-c5ccccc5)nc(-c5ccccc5)n4)c3)c2)cc1 ; c1ccc(-c2ccc(-c3ccccc3)c(-c3cnc(-n4c5ccccc5c5c6sc7ccccc7c6ccc54)c(-c4nc(-c5ccccc5)nc(-c5ccccc5)n4)c3)c2)cc1]

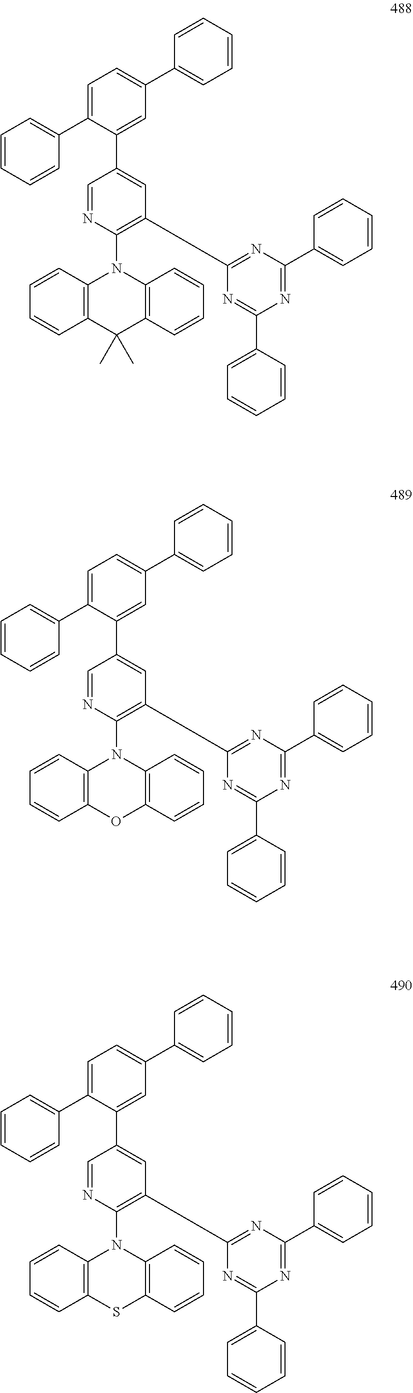 [SMILES: CC1(C)c2ccccc2N(c2ncc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3cnc(N4c5ccccc5Oc5ccccc54)c(-c4nc(-c5ccccc5)nc(-c5ccccc5)n4)c3)c2)cc1 ; c1ccc(-c2ccc(-c3ccccc3)c(-c3cnc(N4c5ccccc5Sc5ccccc54)c(-c4nc(-c5ccccc5)nc(-c5ccccc5)n4)c3)c2)cc1]

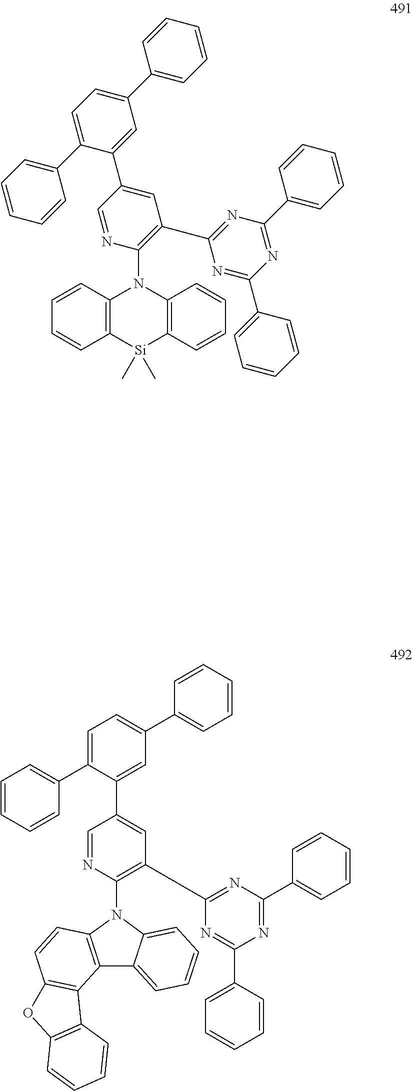 [SMILES: C[Si]1(C)c2ccccc2N(c2ncc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3cnc(-n4c5ccccc5c5c6c(ccc54)oc4ccccc46)c(-c4nc(-c5ccccc5)nc(-c5ccccc5)n4)c3)c2)cc1]

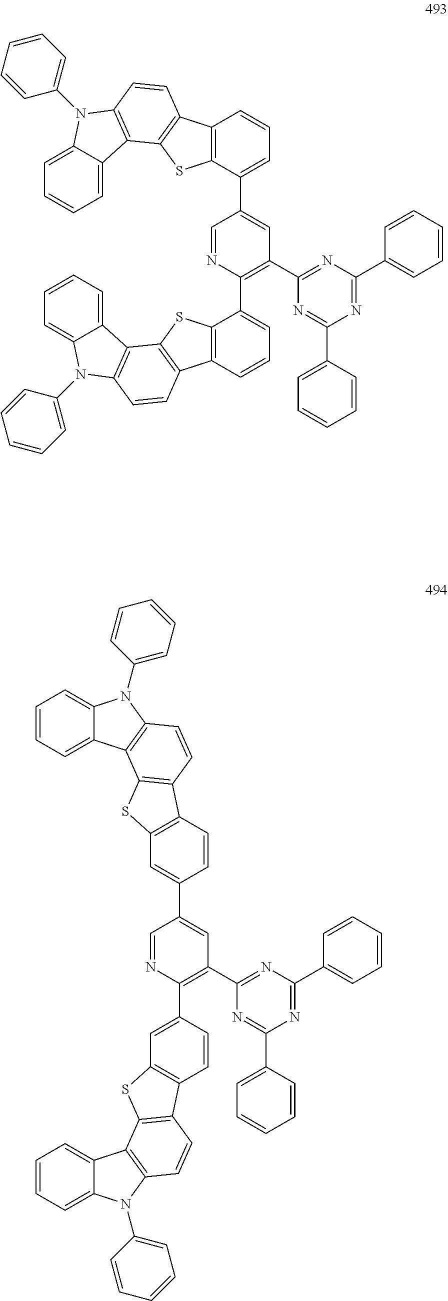 [SMILES: c1ccc(-c2nc(-c3ccccc3)nc(-c3cc(-c4cccc5c4sc4c5ccc5c4c4ccccc4n5-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4ccc5c(c4)sc4c5ccc5c4c4ccccc4n5-c4ccccc4)cnc3-c3ccc4c(c3)sc3c4ccc4c3c3ccccc3n4-c3ccccc3)n2)cc1]

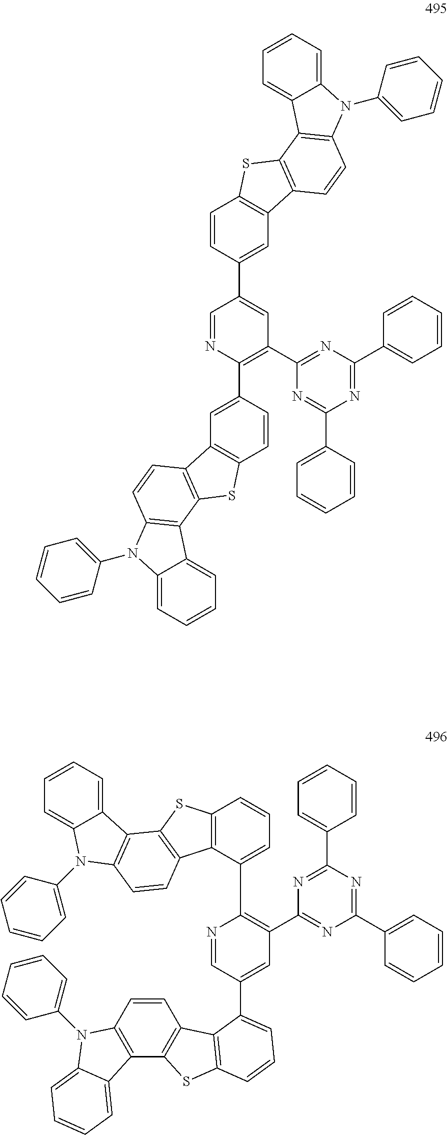 [SMILES: c1ccc(-c2nc(-c3ccccc3)nc(-c3cc(-c4ccc5sc6c(ccc7c6c6ccccc6n7-c6ccccc6)c5c4)cnc3-c3ccc4sc5c(ccc6c5c5ccccc5n6-c5ccccc5)c4c3)n2)cc1 ; c1ccc(-c2nc(-c3ccccc3)nc(-c3cc(-c4cccc5sc6c(ccc7c6c6ccccc6n7-c6ccccc6)c45)cnc3-c3cccc4sc5c(ccc6c5c5ccccc5n6-c5ccccc5)c34)n2)cc1]

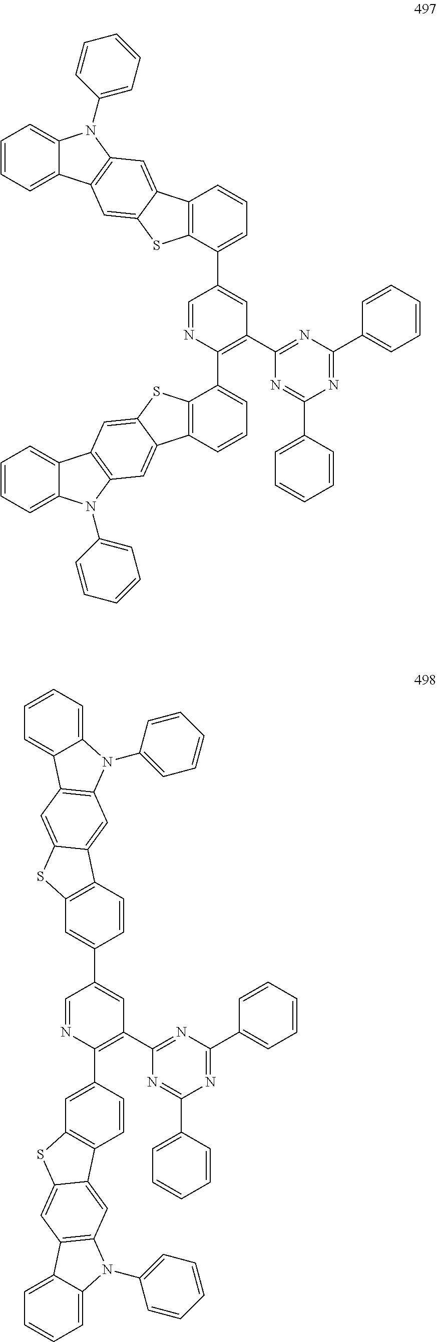 [SMILES: c1ccc(-c2nc(-c3ccccc3)nc(-c3cc(-c4cccc5c4sc4cc6c7ccccc7n(-c7ccccc7)c6cc45)cnc3-c3cccc4c3sc3cc5c6ccccc6n(-c6ccccc6)c5cc34)n2)cc1 ; c1ccc(-c2nc(-c3ccccc3)nc(-c3cc(-c4ccc5c(c4)sc4cc6c7ccccc7n(-c7ccccc7)c6cc45)cnc3-c3ccc4c(c3)sc3cc5c6ccccc6n(-c6ccccc6)c5cc34)n2)cc1]

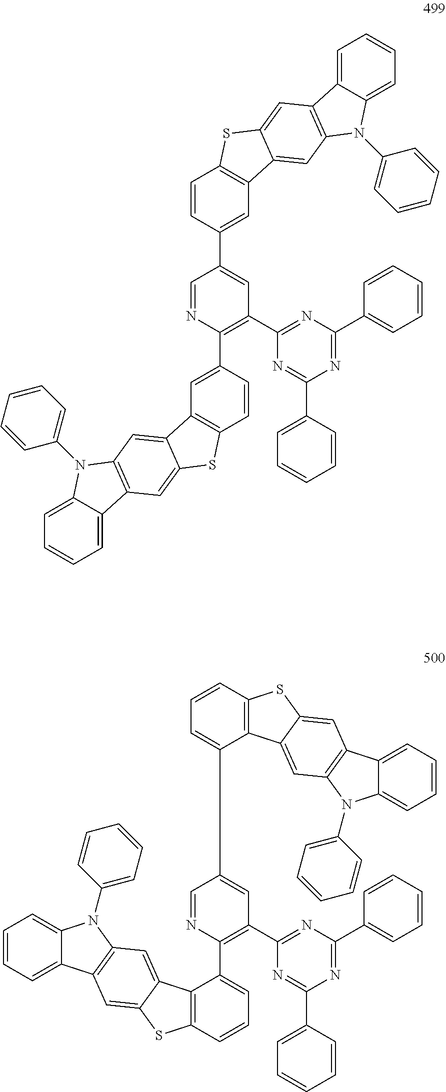 [SMILES: c1ccc(-c2nc(-c3ccccc3)nc(-c3cc(-c4ccc5sc6cc7c8ccccc8n(-c8ccccc8)c7cc6c5c4)cnc3-c3ccc4sc5cc6c7ccccc7n(-c7ccccc7)c6cc5c4c3)n2)cc1 ; c1ccc(-c2nc(-c3ccccc3)nc(-c3cc(-c4cccc5sc6cc7c8ccccc8n(-c8ccccc8)c7cc6c45)cnc3-c3cccc4sc5cc6c7ccccc7n(-c7ccccc7)c6cc5c34)n2)cc1]

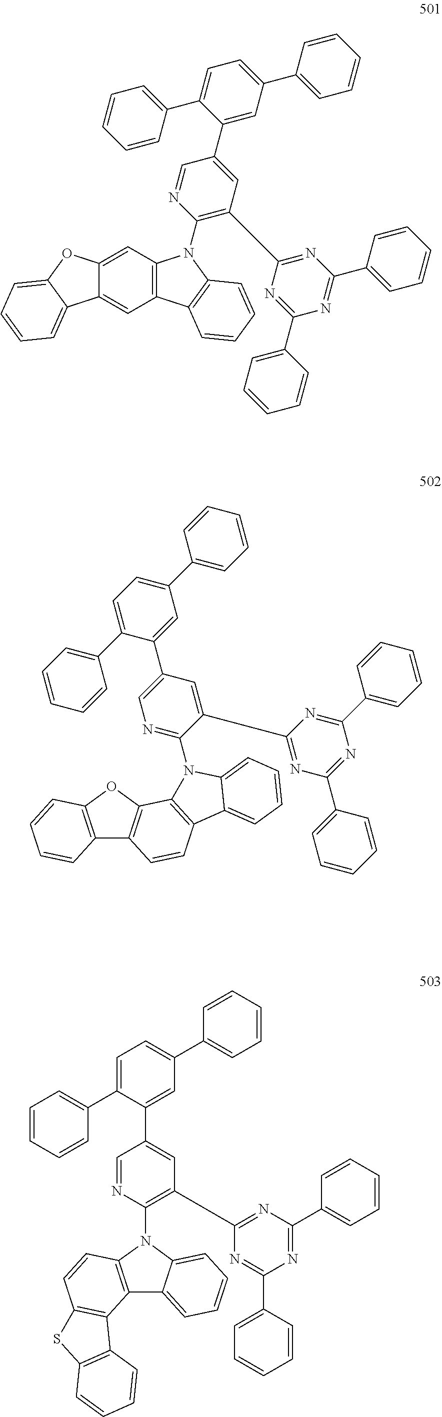 [SMILES: c1ccc(-c2ccc(-c3ccccc3)c(-c3cnc(-n4c5ccccc5c5cc6c(cc54)oc4ccccc46)c(-c4nc(-c5ccccc5)nc(-c5ccccc5)n4)c3)c2)cc1 ; c1ccc(-c2ccc(-c3ccccc3)c(-c3cnc(-n4c5ccccc5c5ccc6c7ccccc7oc6c54)c(-c4nc(-c5ccccc5)nc(-c5ccccc5)n4)c3)c2)cc1 ; c1ccc(-c2ccc(-c3ccccc3)c(-c3cnc(-n4c5ccccc5c5c6c(ccc54)sc4ccccc46)c(-c4nc(-c5ccccc5)nc(-c5ccccc5)n4)c3)c2)cc1]

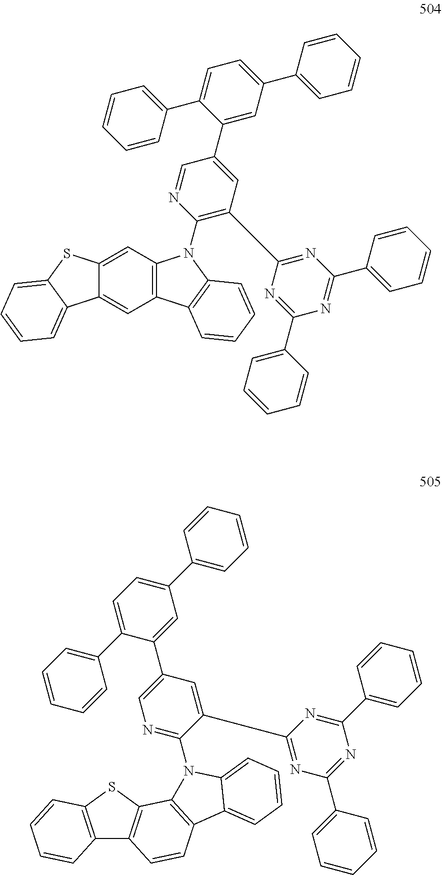 [SMILES: c1ccc(-c2ccc(-c3ccccc3)c(-c3cnc(-n4c5ccccc5c5cc6c(cc54)sc4ccccc46)c(-c4nc(-c5ccccc5)nc(-c5ccccc5)n4)c3)c2)cc1 ; c1ccc(-c2ccc(-c3ccccc3)c(-c3cnc(-n4c5ccccc5c5ccc6c7ccccc7sc6c54)c(-c4nc(-c5ccccc5)nc(-c5ccccc5)n4)c3)c2)cc1]

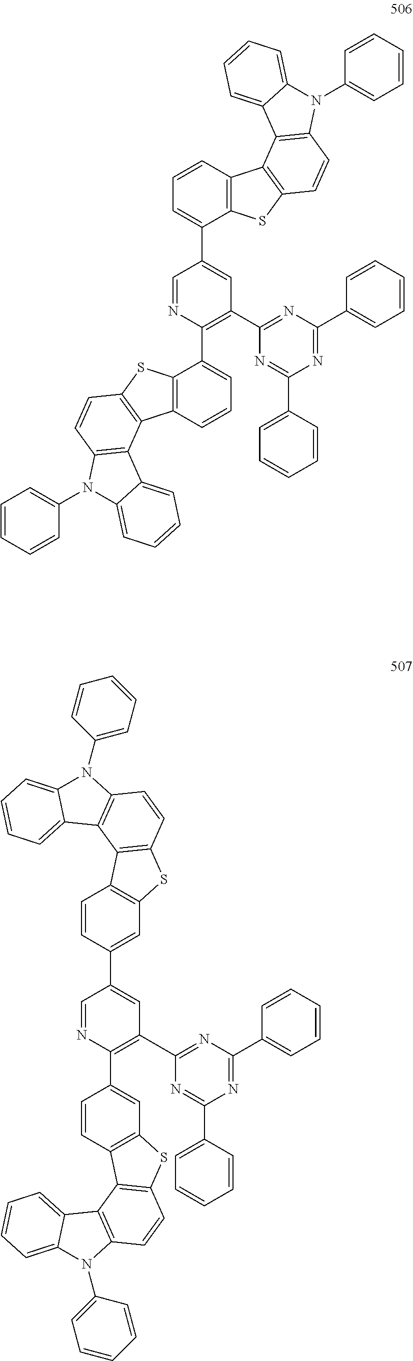 [SMILES: c1ccc(-c2nc(-c3ccccc3)nc(-c3cc(-c4cccc5c4sc4ccc6c(c7ccccc7n6-c6ccccc6)c45)cnc3-c3cccc4c3sc3ccc5c(c6ccccc6n5-c5ccccc5)c34)n2)cc1 ; c1ccc(-c2nc(-c3ccccc3)nc(-c3cc(-c4ccc5c(c4)sc4ccc6c(c7ccccc7n6-c6ccccc6)c45)cnc3-c3ccc4c(c3)sc3ccc5c(c6ccccc6n5-c5ccccc5)c34)n2)cc1]

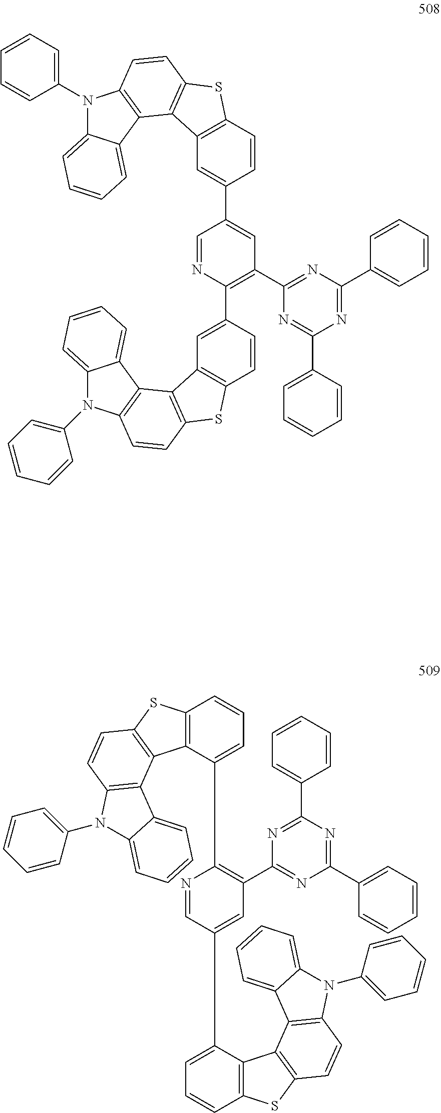 [SMILES: c1ccc(-c2nc(-c3ccccc3)nc(-c3cc(-c4ccc5sc6ccc7c(c8ccccc8n7-c7ccccc7)c6c5c4)cnc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)cnc3-c3cccc4sc5ccc6c(c7ccccc7n6-c6ccccc6)c5c34)n2)cc1]

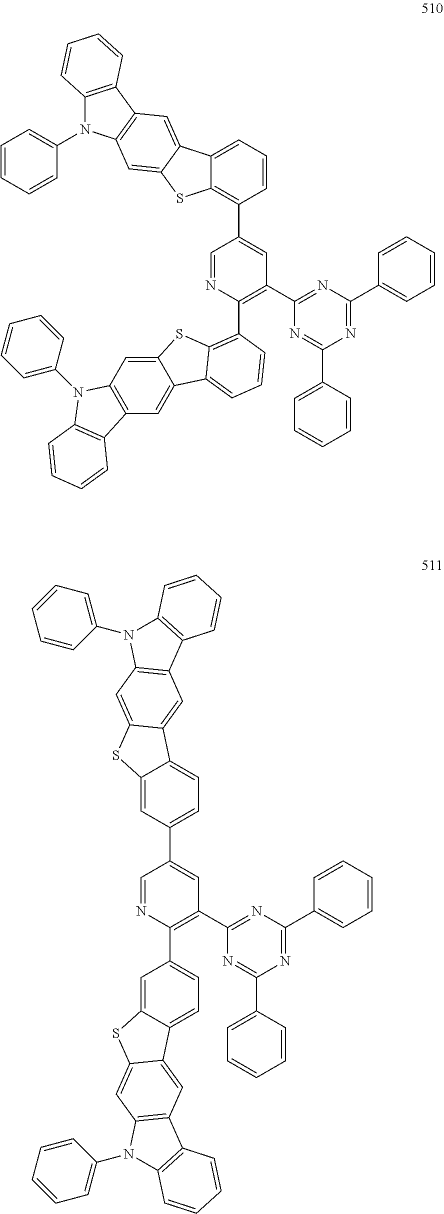 [SMILES: c1ccc(-c2nc(-c3ccccc3)nc(-c3cc(-c4cccc5c4sc4cc6c(cc45)c4ccccc4n6-c4ccccc4)cnc3-c3cccc4c3sc3cc5c(cc34)c3ccccc3n5-c3ccccc3)n2)cc1 ; c1ccc(-c2nc(-c3ccccc3)nc(-c3cc(-c4ccc5c(c4)sc4cc6c(cc45)c4ccccc4n6-c4ccccc4)cnc3-c3ccc4c(c3)sc3cc5c(cc34)c3ccccc3n5-c3ccccc3)n2)cc1]

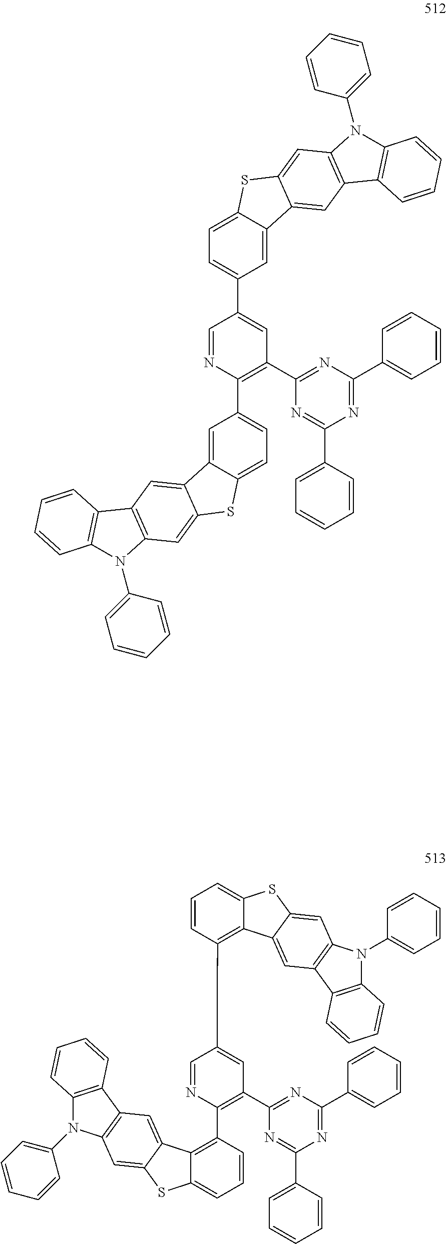 [SMILES: c1ccc(-c2nc(-c3ccccc3)nc(-c3cc(-c4ccc5sc6cc7c(cc6c5c4)c4ccccc4n7-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4cccc5sc6cc7c(cc6c45)c4ccccc4n7-c4ccccc4)cnc3-c3cccc4sc5cc6c(cc5c34)c3ccccc3n6-c3ccccc3)n2)cc1]

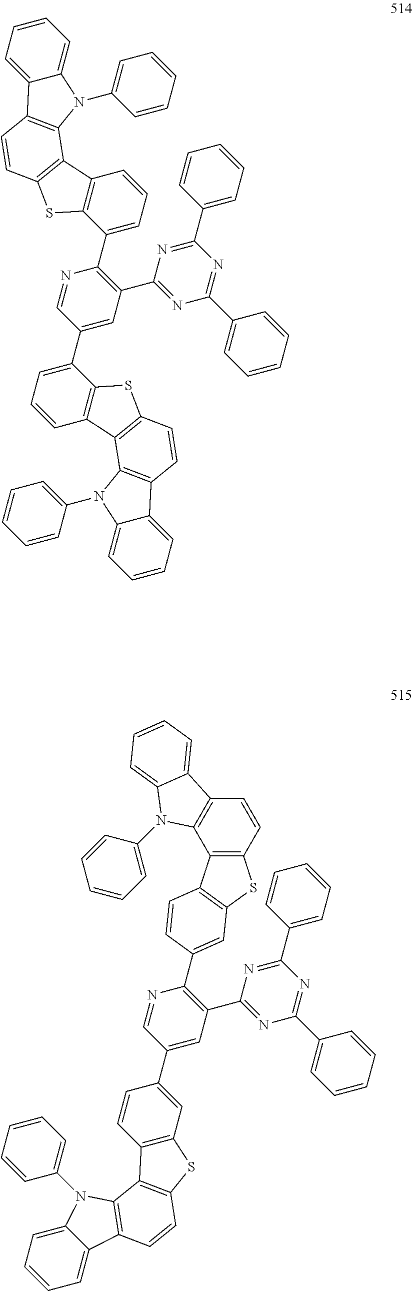 [SMILES: c1ccc(-c2nc(-c3ccccc3)nc(-c3cc(-c4cccc5c4sc4ccc6c7ccccc7n(-c7ccccc7)c6c45)cnc3-c3cccc4c3sc3ccc5c6ccccc6n(-c6ccccc6)c5c34)n2)cc1 ; c1ccc(-c2nc(-c3ccccc3)nc(-c3cc(-c4ccc5c(c4)sc4ccc6c7ccccc7n(-c7ccccc7)c6c45)cnc3-c3ccc4c(c3)sc3ccc5c6ccccc6n(-c6ccccc6)c5c34)n2)cc1]

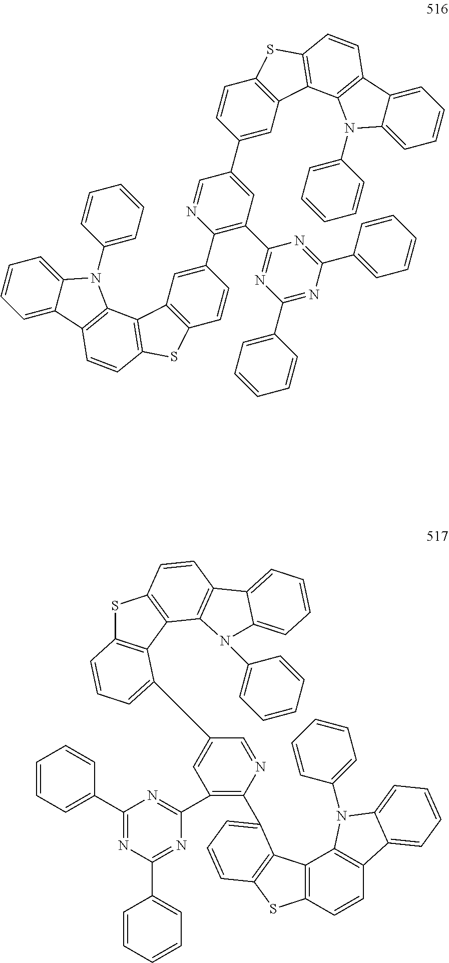 [SMILES: c1ccc(-c2nc(-c3ccccc3)nc(-c3cc(-c4ccc5sc6ccc7c8ccccc8n(-c8ccccc8)c7c6c5c4)cnc3-c3ccc4sc5ccc6c7ccccc7n(-c7ccccc7)c6c5c4c3)n2)cc1 ; c1ccc(-c2nc(-c3ccccc3)nc(-c3cc(-c4cccc5sc6ccc7c8ccccc8n(-c8ccccc8)c7c6c45)cnc3-c3cccc4sc5ccc6c7ccccc7n(-c7ccccc7)c6c5c34)n2)cc1]

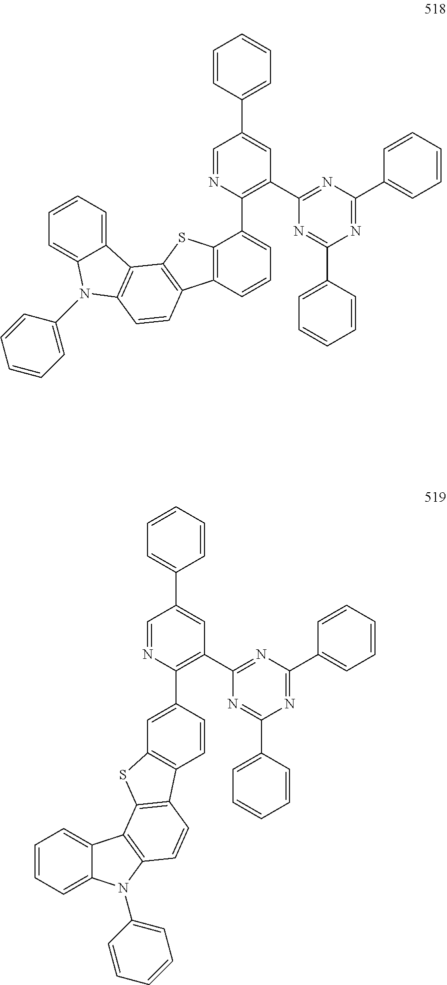 [SMILES: c1ccc(-c2cnc(-c3cccc4c3sc3c4ccc4c3c3ccccc3n4-c3ccccc3)c(-c3nc(-c4ccccc4)nc(-c4ccccc4)n3)c2)cc1 ; c1ccc(-c2cnc(-c3ccc4c(c3)sc3c4ccc4c3c3ccccc3n4-c3ccccc3)c(-c3nc(-c4ccccc4)nc(-c4ccccc4)n3)c2)cc1]

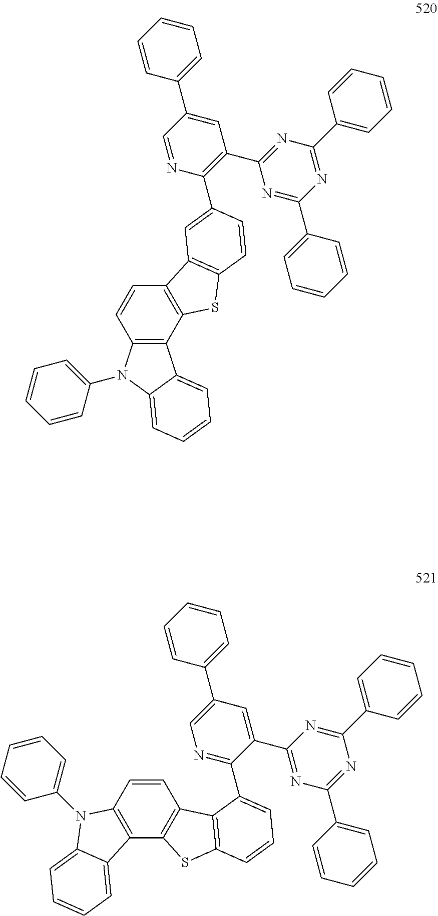 [SMILES: c1ccc(-c2cnc(-c3ccc4sc5c(ccc6c5c5ccccc5n6-c5ccccc5)c4c3)c(-c3nc(-c4ccccc4)nc(-c4ccccc4)n3)c2)cc1 ; c1ccc(-c2cnc(-c3cccc4sc5c(ccc6c5c5ccccc5n6-c5ccccc5)c34)c(-c3nc(-c4ccccc4)nc(-c4ccccc4)n3)c2)cc1]

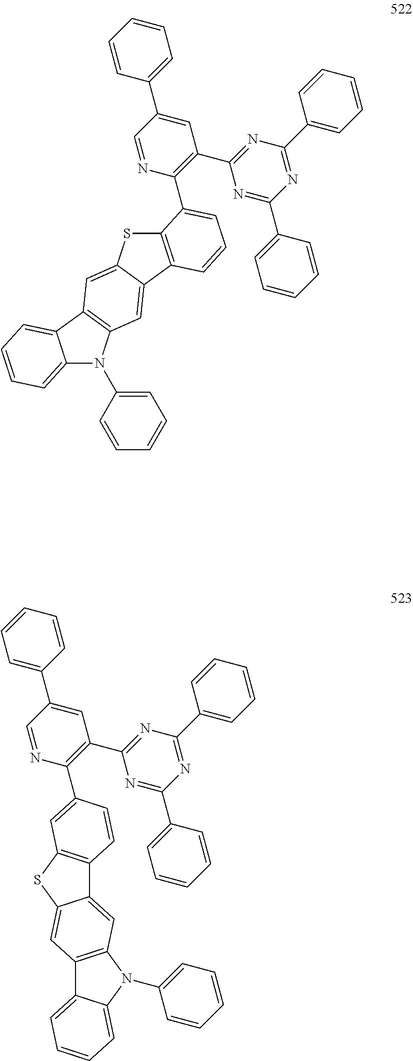 [SMILES: c1ccc(-c2cnc(-c3cccc4c3sc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4c(c3)sc3cc5c6ccccc6n(-c6ccccc6)c5cc34)c(-c3nc(-c4ccccc4)nc(-c4ccccc4)n3)c2)cc1]

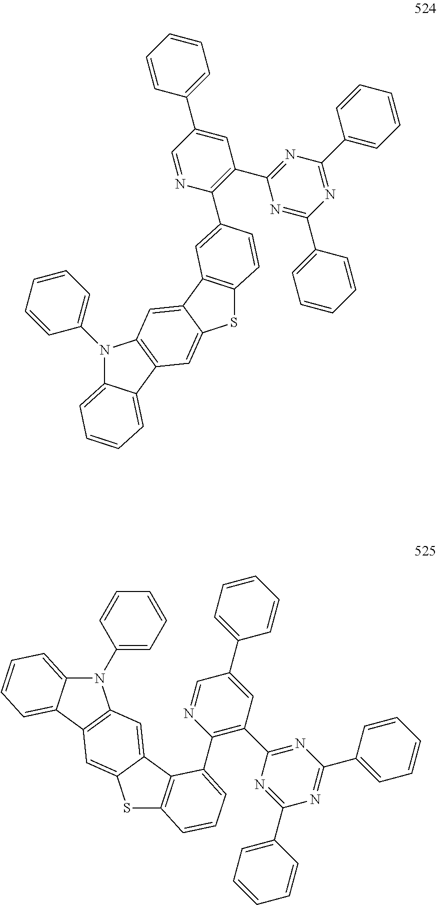 [SMILES: c1ccc(-c2cnc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4sc5cc6c7ccccc7n(-c7ccccc7)c6cc5c34)c(-c3nc(-c4ccccc4)nc(-c4ccccc4)n3)c2)cc1]

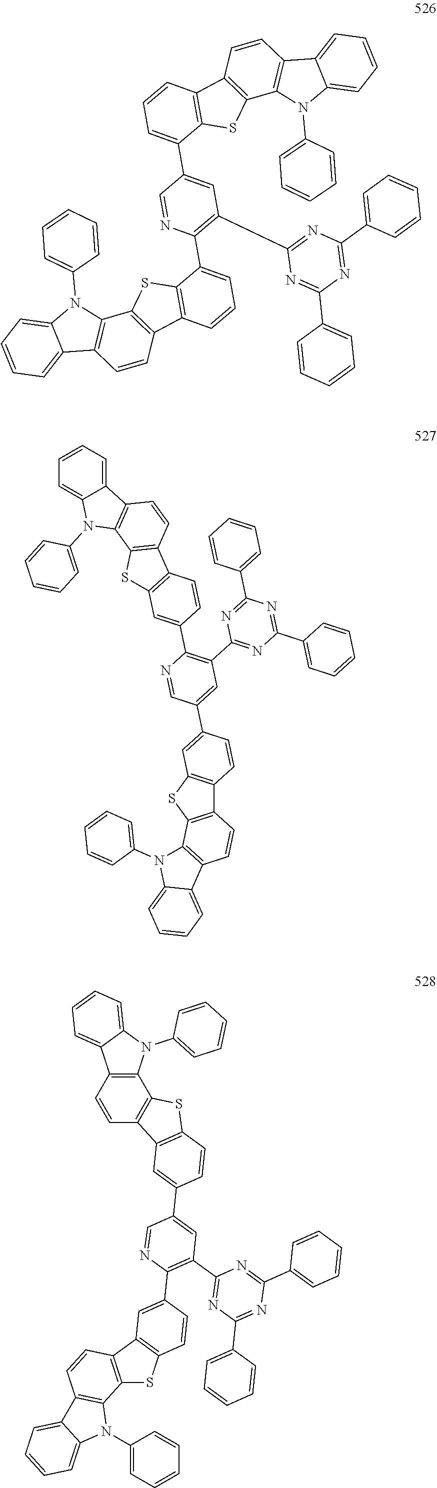 [SMILES: c1ccc(-c2nc(-c3ccccc3)nc(-c3cc(-c4cccc5c4sc4c5ccc5c6ccccc6n(-c6ccccc6)c54)cnc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)cnc3-c3ccc4c(c3)sc3c4ccc4c5ccccc5n(-c5ccccc5)c43)n2)cc1 ; c1ccc(-c2nc(-c3ccccc3)nc(-c3cc(-c4ccc5sc6c(ccc7c8ccccc8n(-c8ccccc8)c76)c5c4)cnc3-c3ccc4sc5c(ccc6c7ccccc7n(-c7ccccc7)c65)c4c3)n2)cc1]

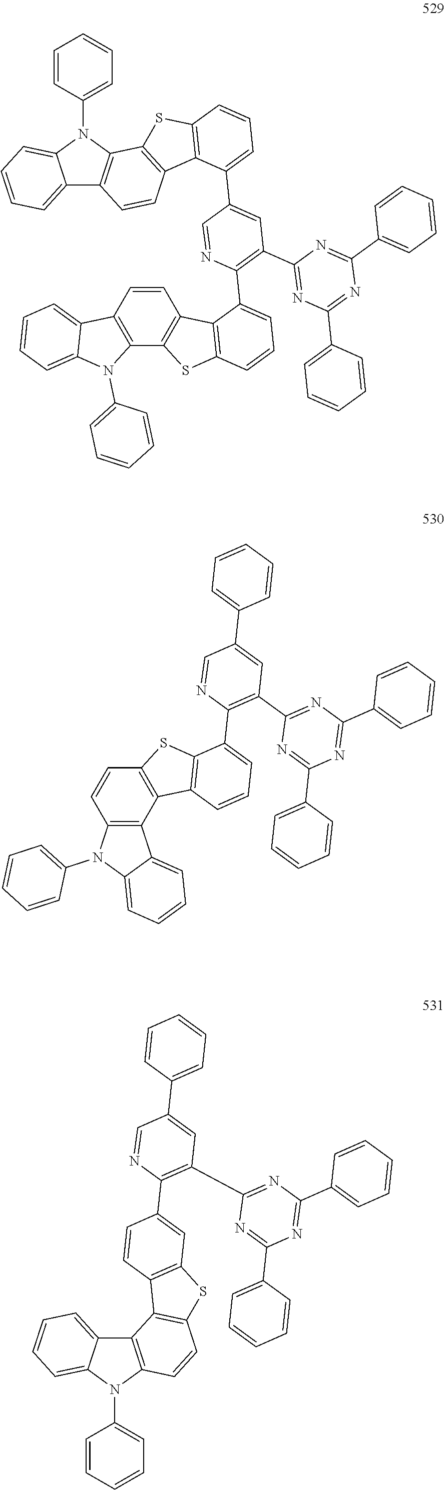 [SMILES: c1ccc(-c2nc(-c3ccccc3)nc(-c3cc(-c4cccc5sc6c(ccc7c8ccccc8n(-c8ccccc8)c76)c45)cnc3-c3cccc4sc5c(ccc6c7ccccc7n(-c7ccccc7)c65)c34)n2)cc1 ; c1ccc(-c2cnc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)sc3ccc5c(c6ccccc6n5-c5ccccc5)c34)c(-c3nc(-c4ccccc4)nc(-c4ccccc4)n3)c2)cc1]

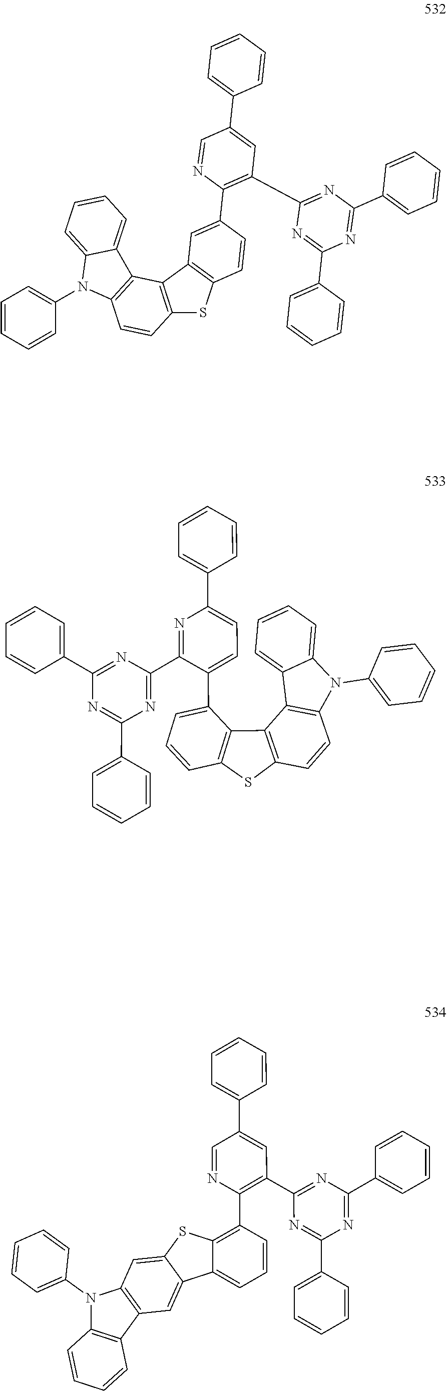 [SMILES: c1ccc(-c2cnc(-c3ccc4sc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4sc5ccc6c(c7ccccc7n6-c6ccccc6)c5c34)c(-c3nc(-c4ccccc4)nc(-c4ccccc4)n3)n2)cc1 ; c1ccc(-c2cnc(-c3cccc4c3sc3cc5c(cc34)c3ccccc3n5-c3ccccc3)c(-c3nc(-c4ccccc4)nc(-c4ccccc4)n3)c2)cc1]

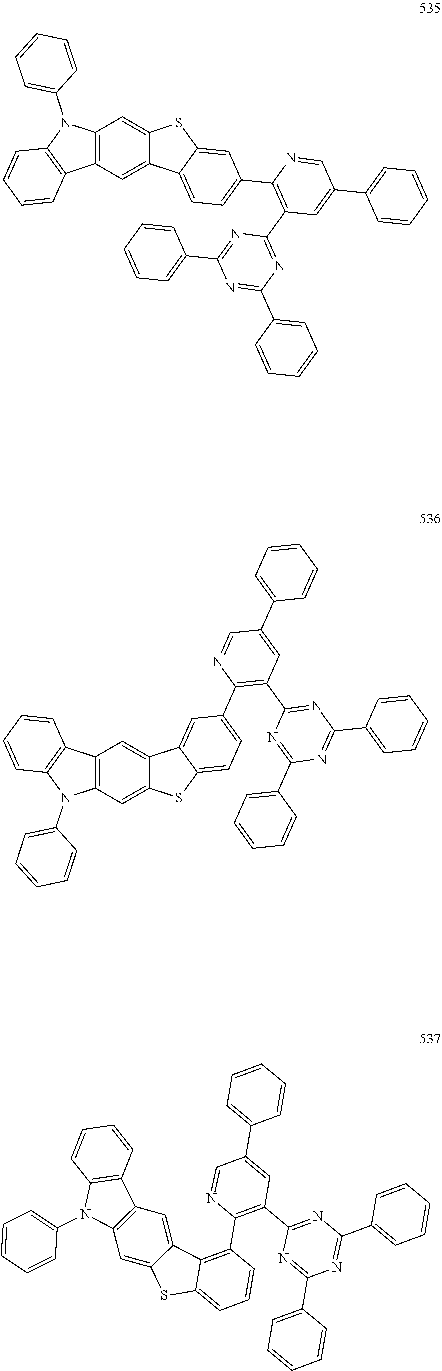 [SMILES: c1ccc(-c2cnc(-c3ccc4c(c3)sc3cc5c(cc34)c3ccccc3n5-c3ccccc3)c(-c3nc(-c4ccccc4)nc(-c4ccccc4)n3)c2)cc1 ; c1ccc(-c2cnc(-c3ccc4sc5cc6c(cc5c4c3)c3ccccc3n6-c3ccccc3)c(-c3nc(-c4ccccc4)nc(-c4ccccc4)n3)c2)cc1 ; c1ccc(-c2cnc(-c3cccc4sc5cc6c(cc5c34)c3ccccc3n6-c3ccccc3)c(-c3nc(-c4ccccc4)nc(-c4ccccc4)n3)c2)cc1]

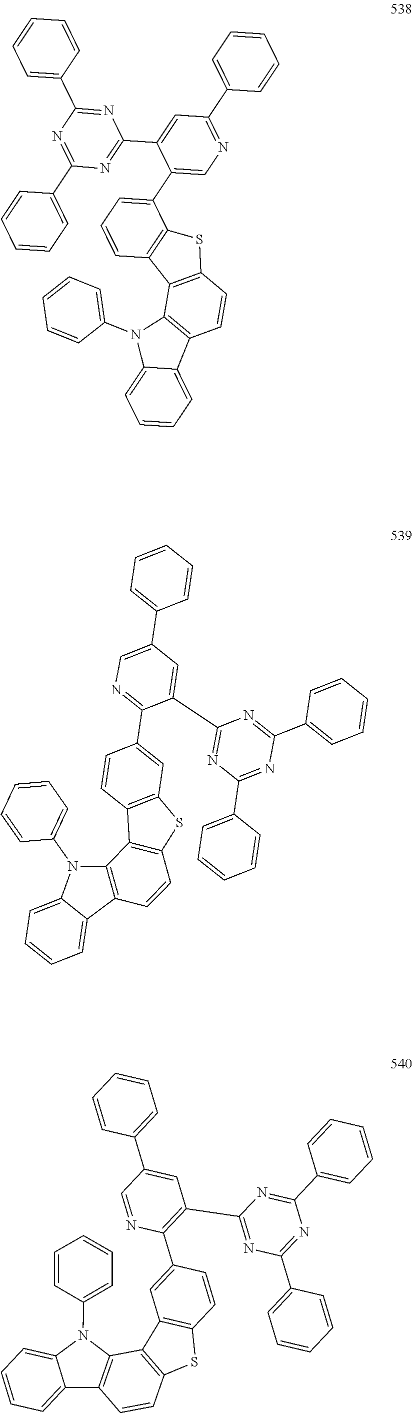 [SMILES: c1ccc(-c2cc(-c3nc(-c4ccccc4)nc(-c4ccccc4)n3)c(-c3cccc4c3sc3ccc5c6ccccc6n(-c6ccccc6)c5c34)cn2)cc1 ; c1ccc(-c2cnc(-c3ccc4c(c3)sc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4sc5ccc6c7ccccc7n(-c7ccccc7)c6c5c4c3)c(-c3nc(-c4ccccc4)nc(-c4ccccc4)n3)c2)cc1]

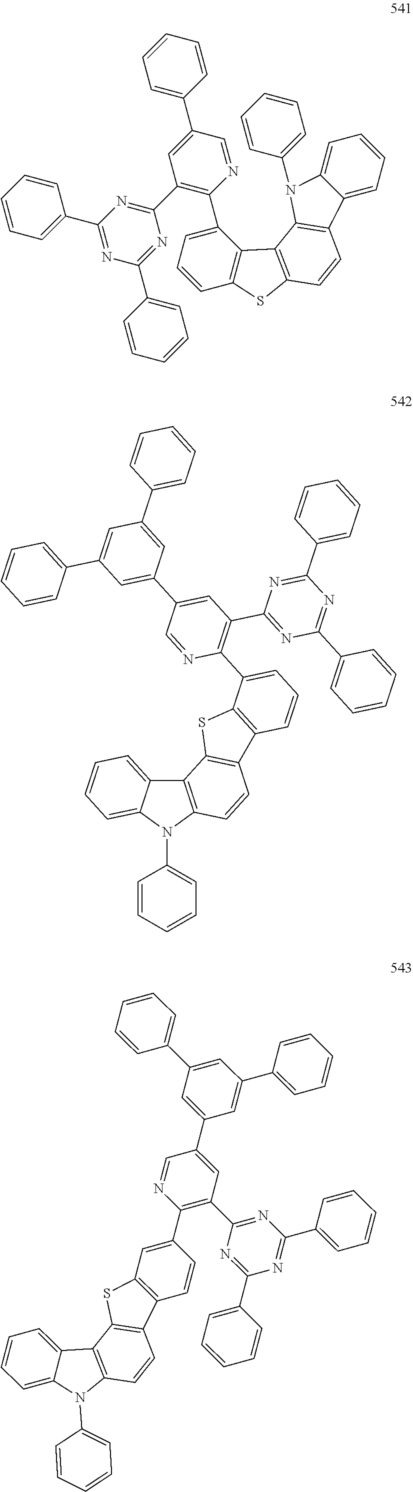 [SMILES: c1ccc(-c2cnc(-c3cccc4sc5ccc6c7ccccc7n(-c7ccccc7)c6c5c34)c(-c3nc(-c4ccccc4)nc(-c4ccccc4)n3)c2)cc1 ; c1ccc(-c2cc(-c3ccccc3)cc(-c3cnc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1]

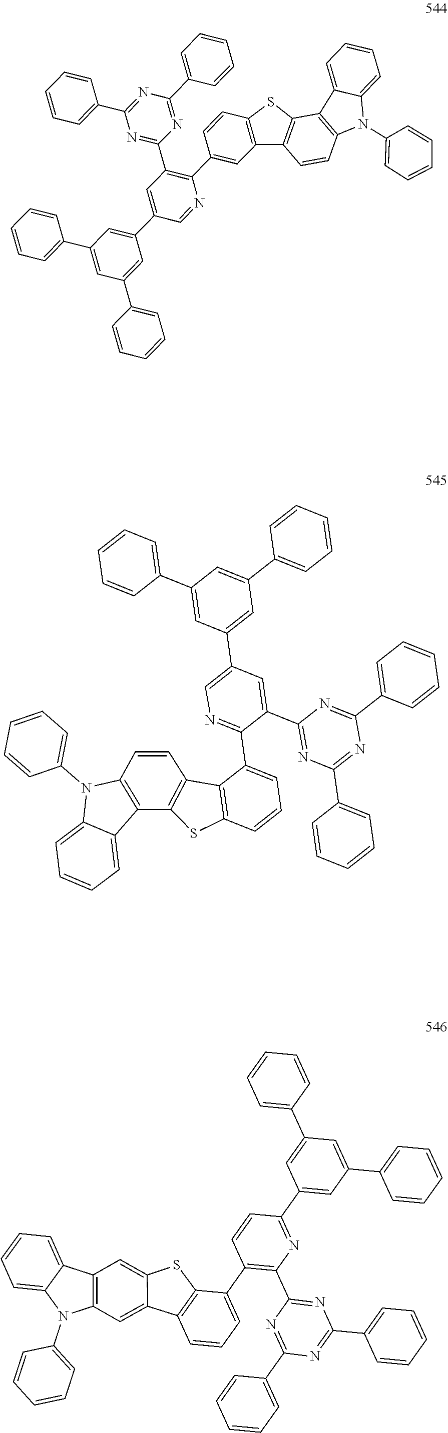 [SMILES: c1ccc(-c2cc(-c3ccccc3)cc(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4cc6c7ccccc7n(-c7ccccc7)c6cc45)c(-c4nc(-c5ccccc5)nc(-c5ccccc5)n4)n3)c2)cc1]

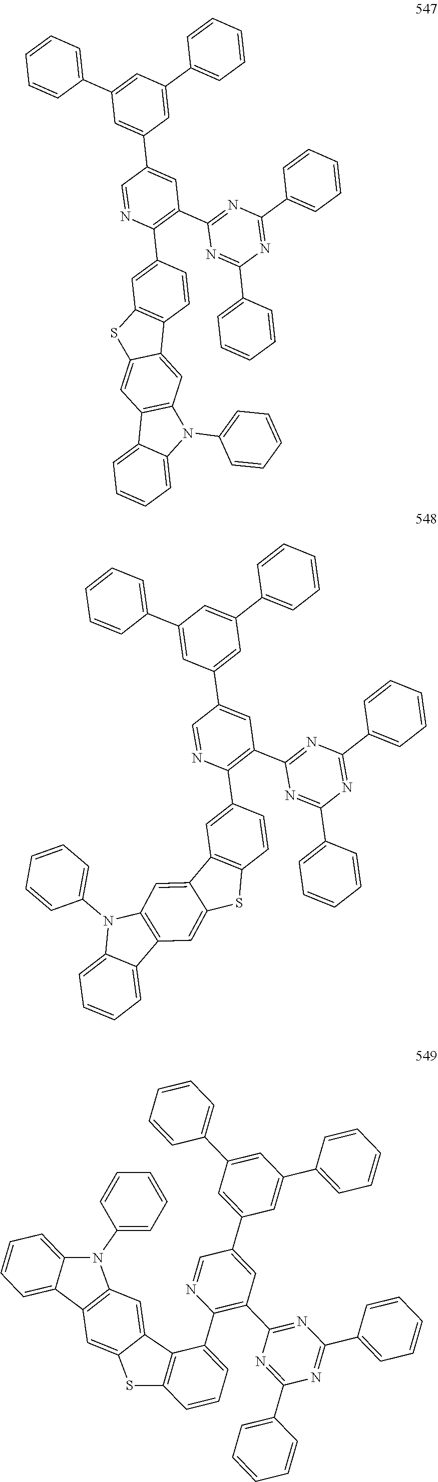 [SMILES: c1ccc(-c2cc(-c3ccccc3)cc(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6cc7c8ccccc8n(-c8ccccc8)c7cc6c45)c(-c4nc(-c5ccccc5)nc(-c5ccccc5)n4)c3)c2)cc1]

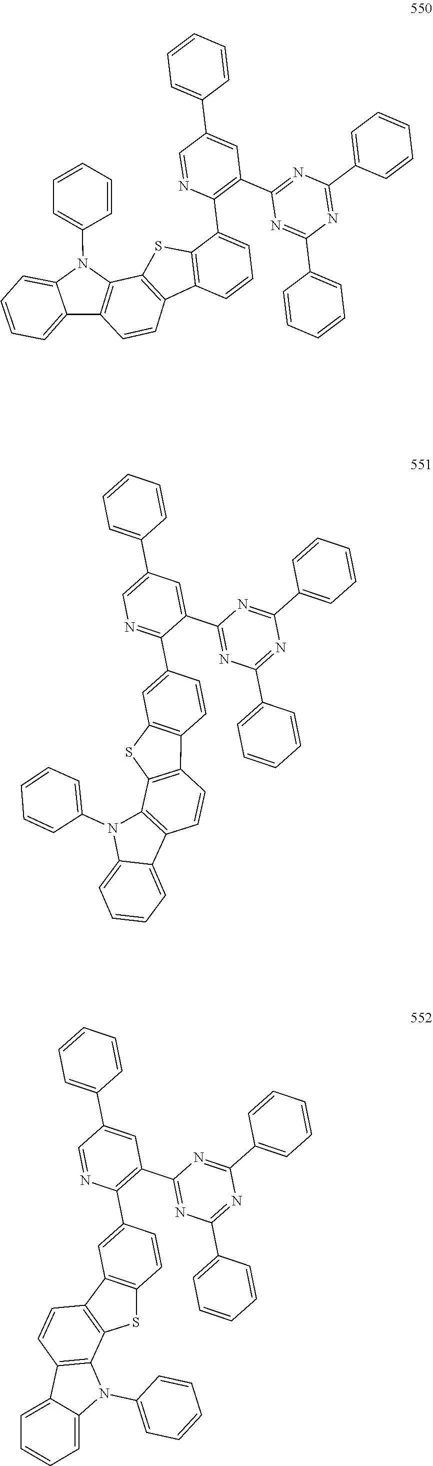 [SMILES: c1ccc(-c2cnc(-c3cccc4c3sc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4c(c3)sc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4sc5c(ccc6c7ccccc7n(-c7ccccc7)c65)c4c3)c(-c3nc(-c4ccccc4)nc(-c4ccccc4)n3)c2)cc1]

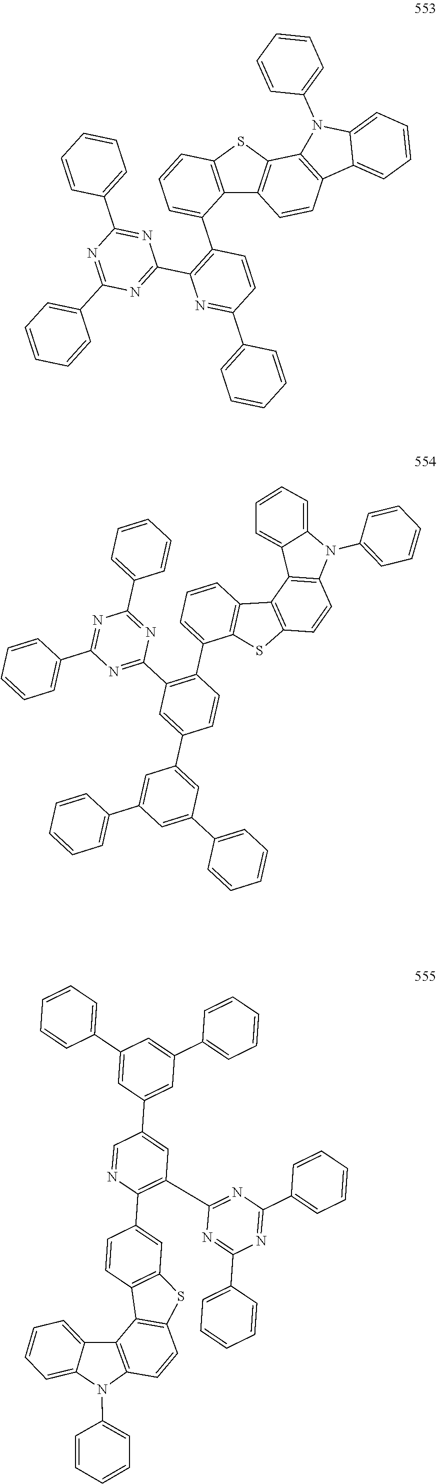 [SMILES: c1ccc(-c2ccc(-c3cccc4sc5c(ccc6c7ccccc7n(-c7ccccc7)c65)c34)c(-c3nc(-c4ccccc4)nc(-c4ccccc4)n3)n2)cc1 ; c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1]

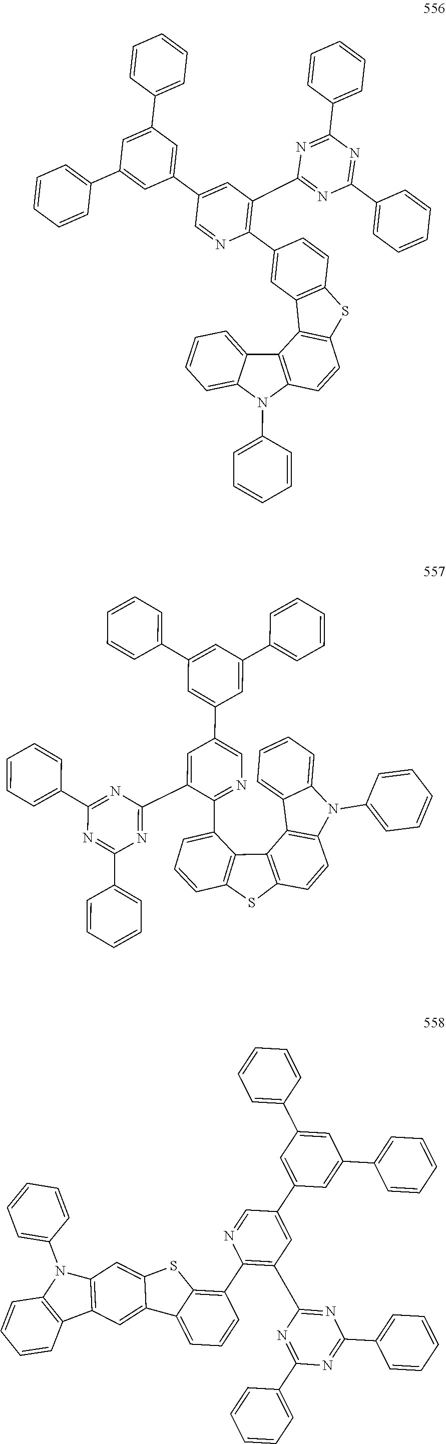 [SMILES: c1ccc(-c2cc(-c3ccccc3)cc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4cccc5c4sc4cc6c(cc45)c4ccccc4n6-c4ccccc4)c(-c4nc(-c5ccccc5)nc(-c5ccccc5)n4)c3)c2)cc1]

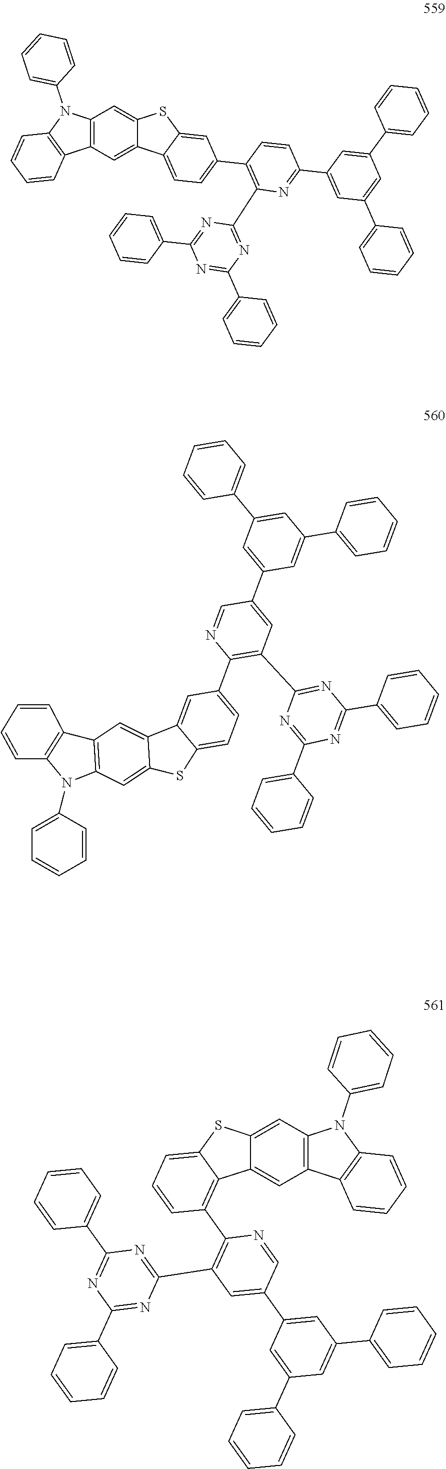 [SMILES: c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)n3)c2)cc1 ; c1ccc(-c2cc(-c3ccccc3)cc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3cnc(-c4cccc5sc6cc7c(cc6c45)c4ccccc4n7-c4ccccc4)c(-c4nc(-c5ccccc5)nc(-c5ccccc5)n4)c3)c2)cc1]

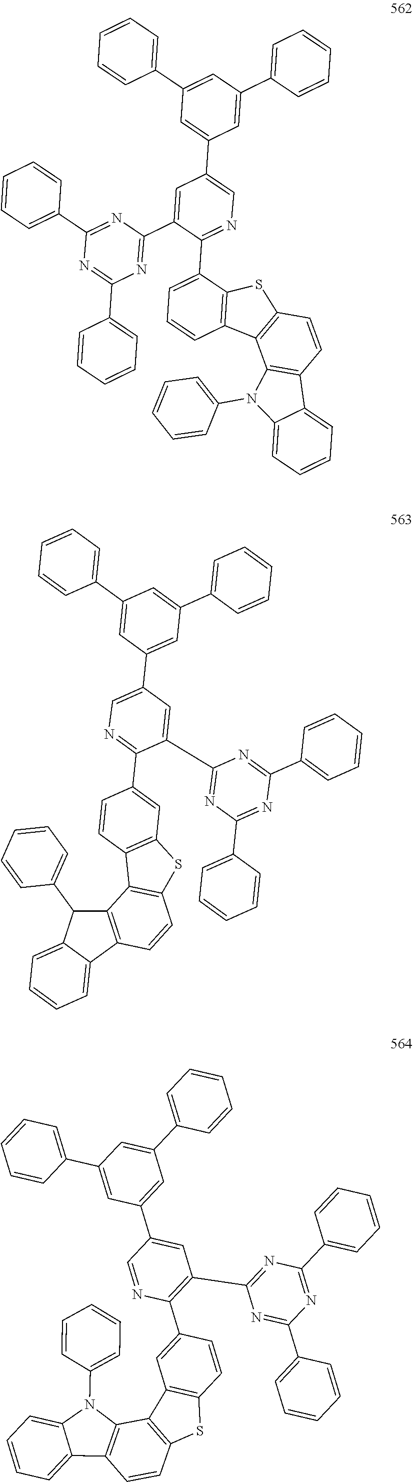 [SMILES: c1ccc(-c2cc(-c3ccccc3)cc(-c3cnc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4ccc6c(c45)C(c4ccccc4)c4ccccc4-6)c(-c4nc(-c5ccccc5)nc(-c5ccccc5)n4)c3)c2)cc1 ; c1ccc(-c2cc(-c3ccccc3)cc(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1]

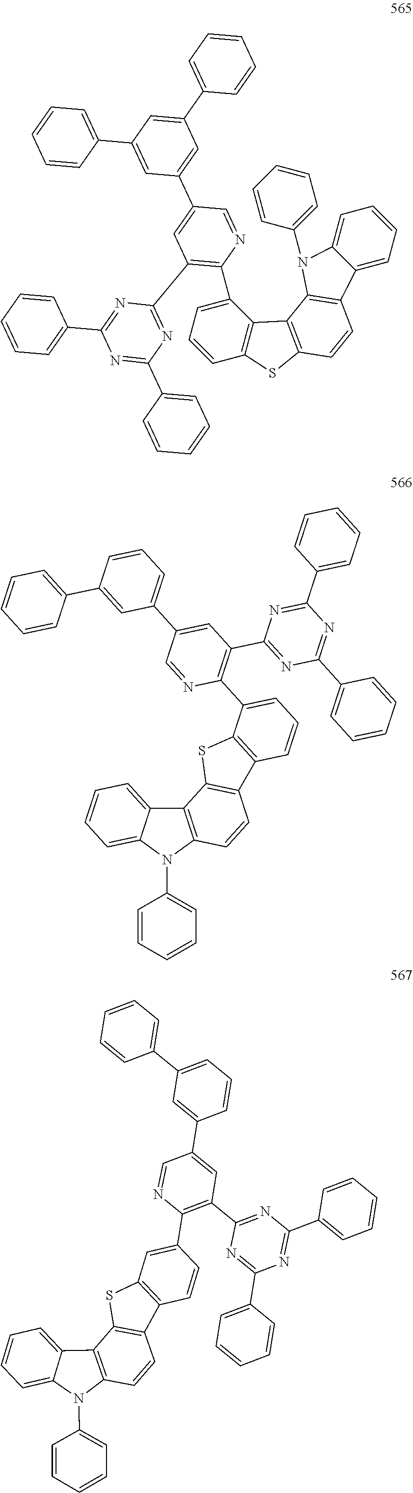 [SMILES: c1ccc(-c2cc(-c3ccccc3)cc(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2cccc(-c3cnc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2cccc(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1]

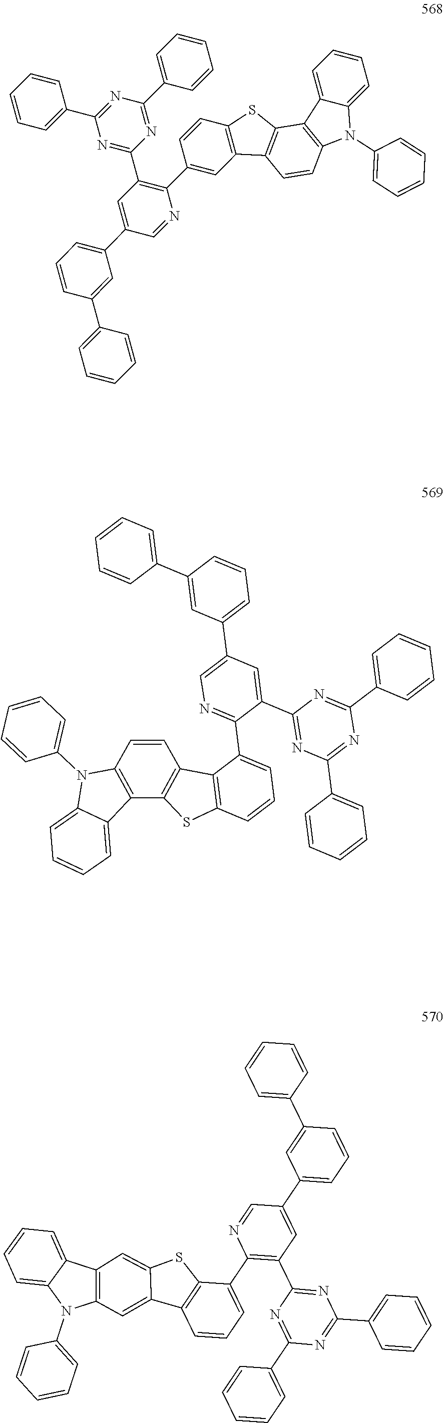 [SMILES: c1ccc(-c2cccc(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3cnc(-c4cccc5c4sc4cc6c7ccccc7n(-c7ccccc7)c6cc45)c(-c4nc(-c5ccccc5)nc(-c5ccccc5)n4)c3)c2)cc1]

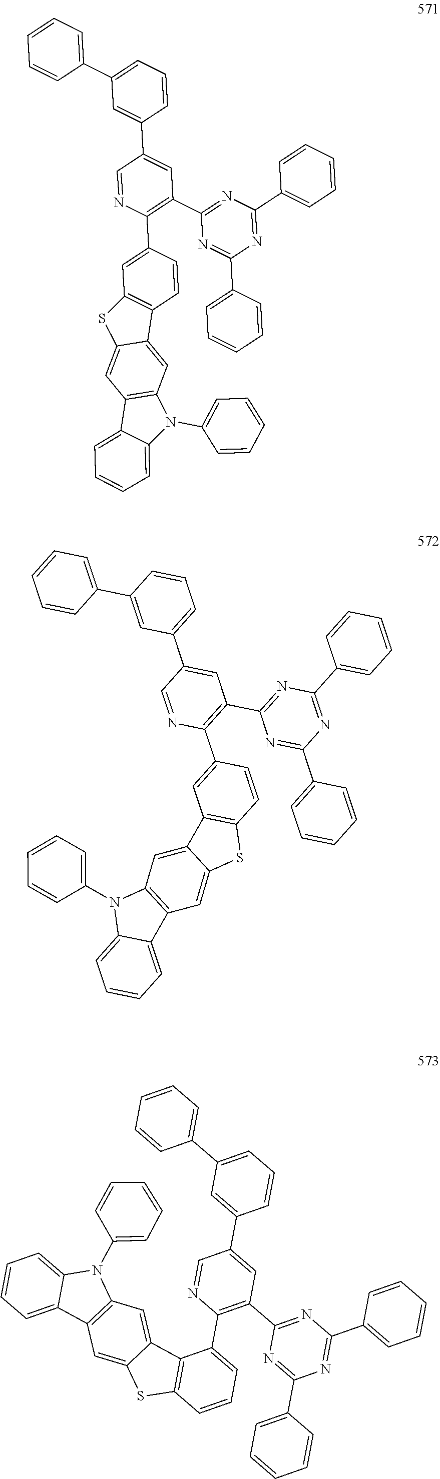 [SMILES: c1ccc(-c2cccc(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3cnc(-c4cccc5sc6cc7c8ccccc8n(-c8ccccc8)c7cc6c45)c(-c4nc(-c5ccccc5)nc(-c5ccccc5)n4)c3)c2)cc1]

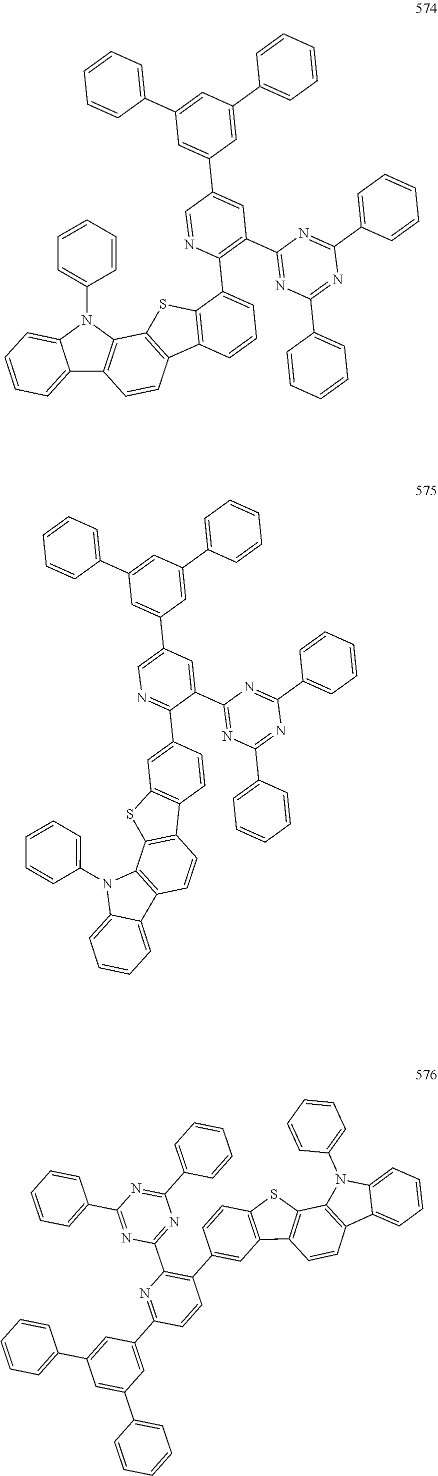 [SMILES: c1ccc(-c2cc(-c3ccccc3)cc(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)n3)c2)cc1]

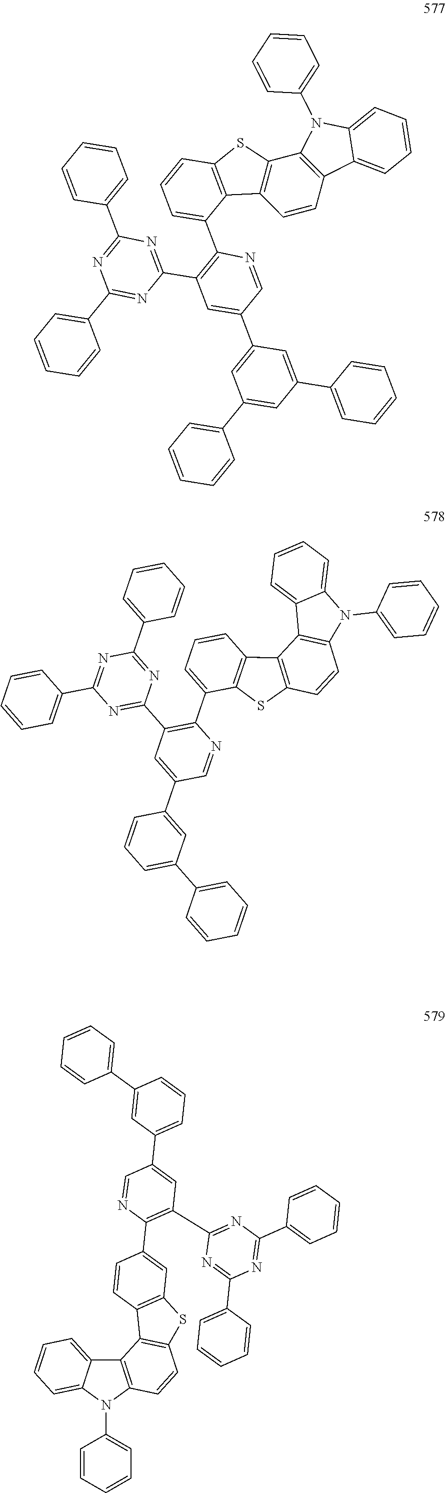 [SMILES: c1ccc(-c2cc(-c3ccccc3)cc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3cnc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1]

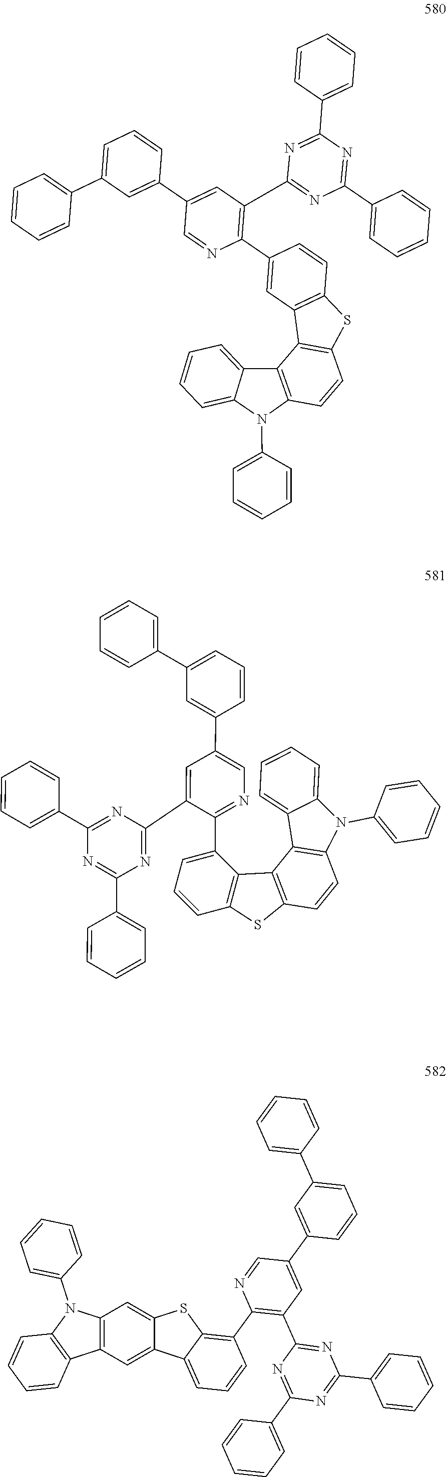 [SMILES: c1ccc(-c2cccc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3cnc(-c4cccc5c4sc4cc6c(cc45)c4ccccc4n6-c4ccccc4)c(-c4nc(-c5ccccc5)nc(-c5ccccc5)n4)c3)c2)cc1]

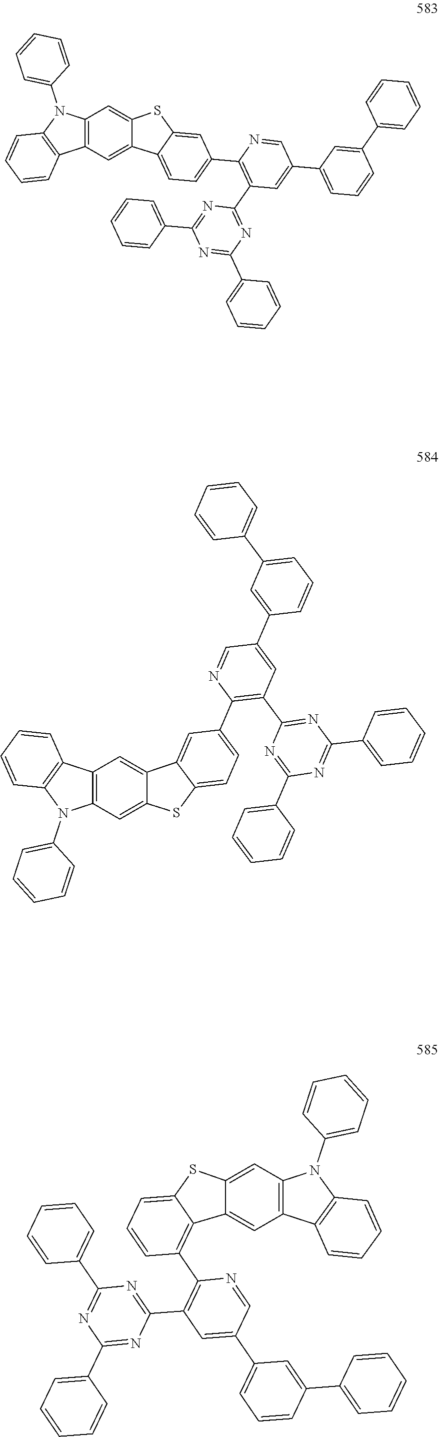 [SMILES: c1ccc(-c2cccc(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3cnc(-c4cccc5sc6cc7c(cc6c45)c4ccccc4n7-c4ccccc4)c(-c4nc(-c5ccccc5)nc(-c5ccccc5)n4)c3)c2)cc1]

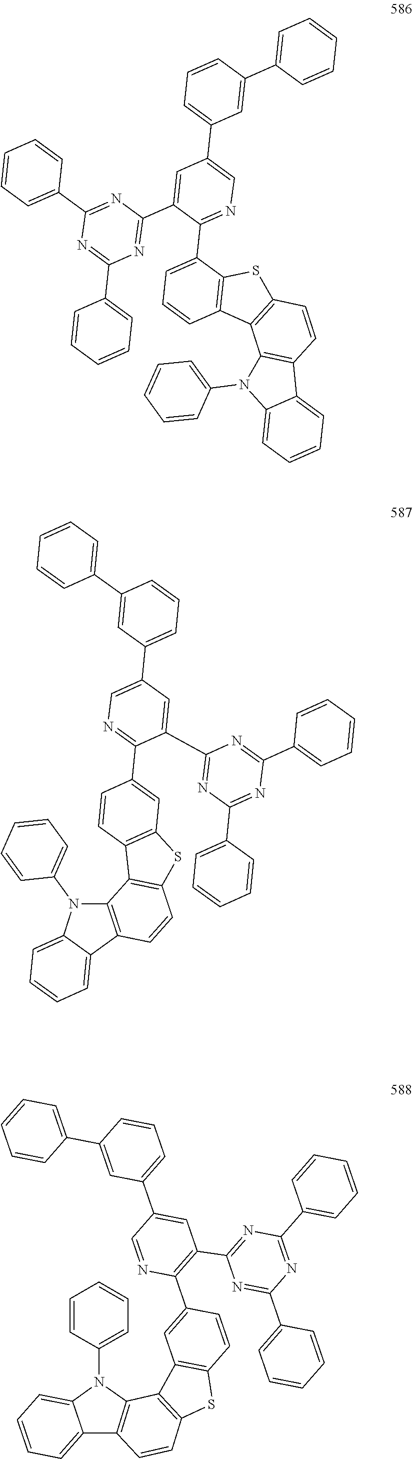 [SMILES: c1ccc(-c2cccc(-c3cnc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1]

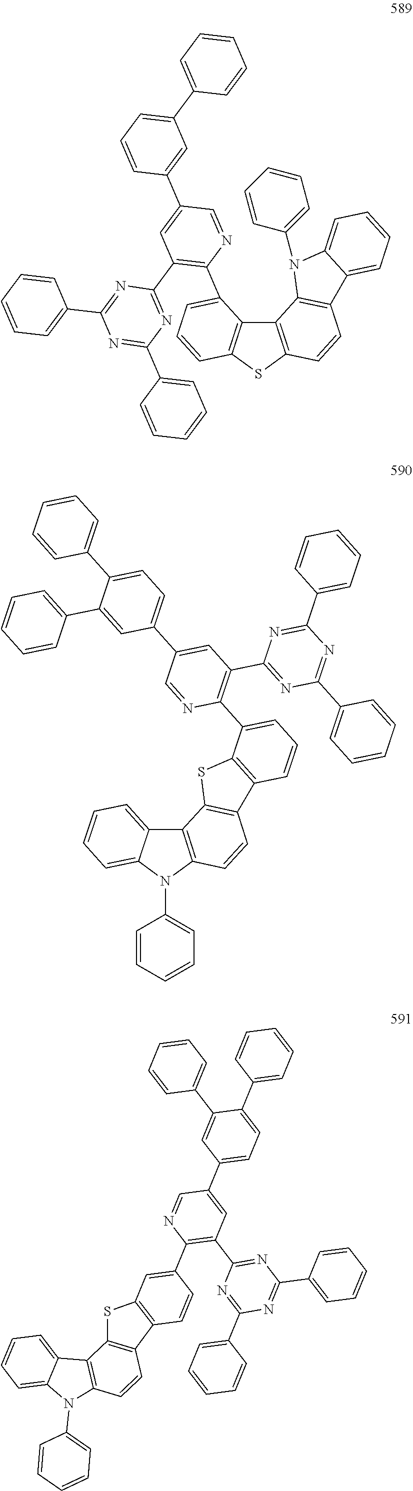 [SMILES: c1ccc(-c2cccc(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3sc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c3c3ccccc3n4-c3ccccc3)n2)cc1]

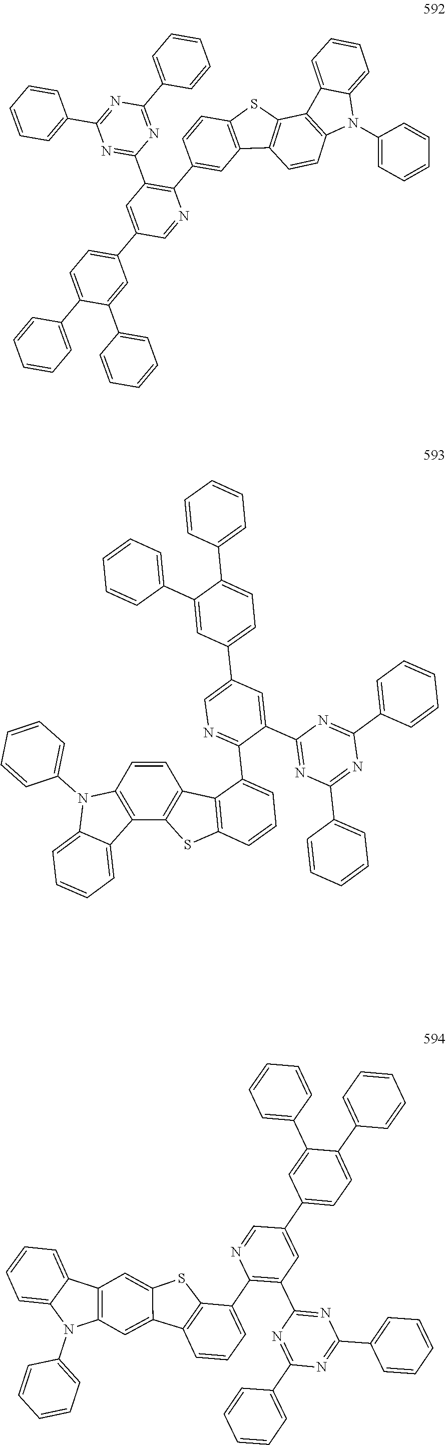 [SMILES: c1ccc(-c2nc(-c3ccccc3)nc(-c3cc(-c4ccc(-c5ccccc5)c(-c5ccccc5)c4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c6ccccc6n(-c6ccccc6)c5cc34)n2)cc1]

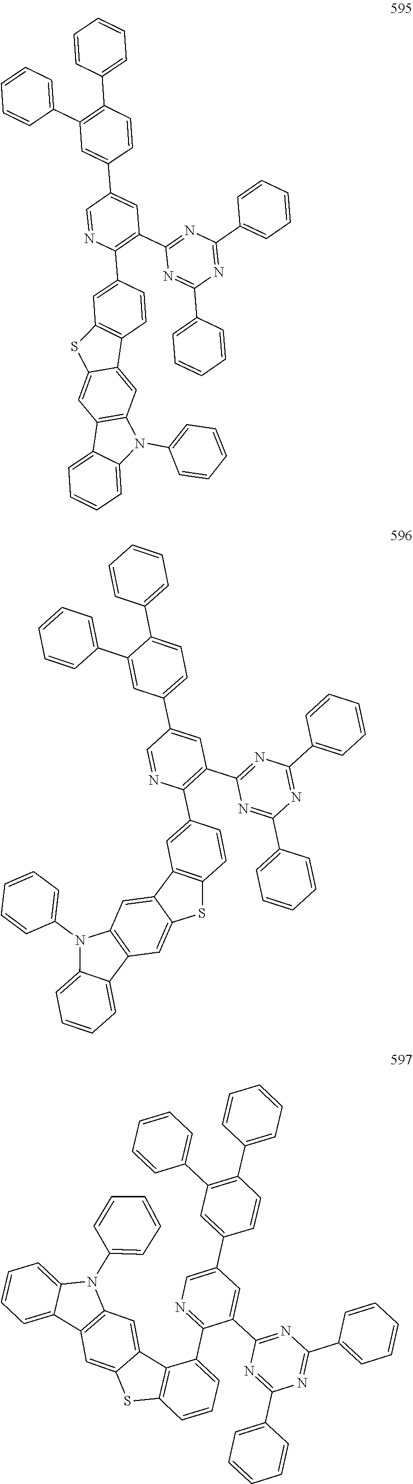 [SMILES: c1ccc(-c2nc(-c3ccccc3)nc(-c3cc(-c4ccc(-c5ccccc5)c(-c5ccccc5)c4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)cnc3-c3cccc4sc5cc6c7ccccc7n(-c7ccccc7)c6cc5c34)n2)cc1]

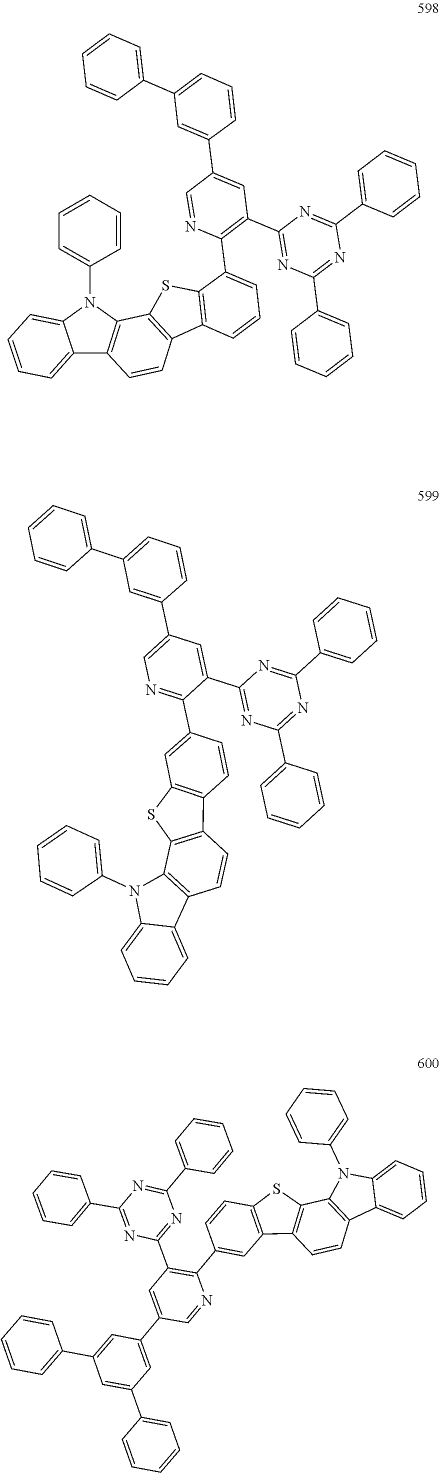 [SMILES: c1ccc(-c2cccc(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1]

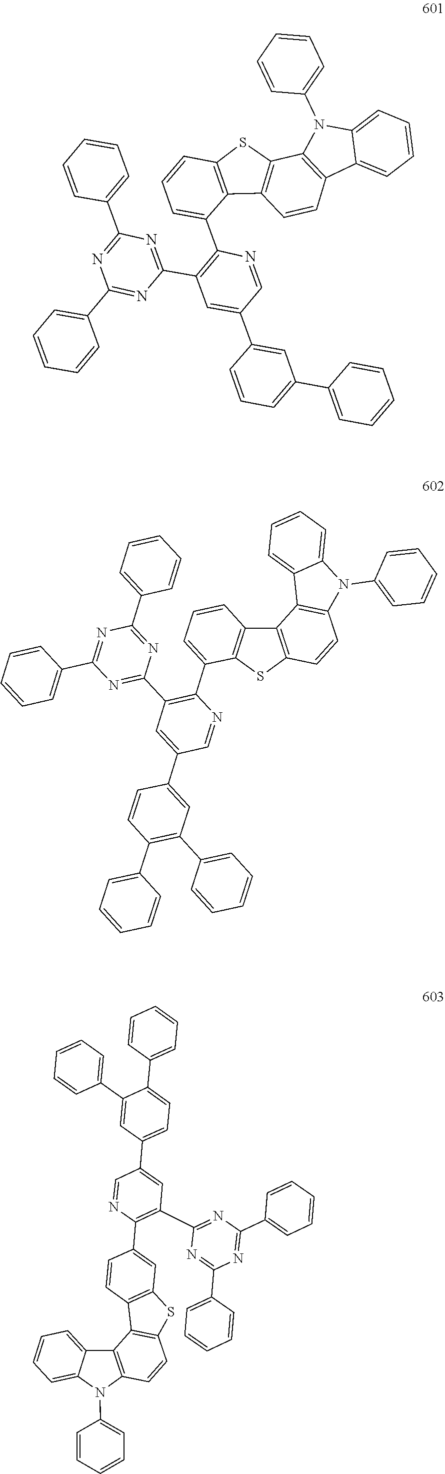 [SMILES: c1ccc(-c2cccc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3sc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c(c6ccccc6n5-c5ccccc5)c34)n2)cc1]

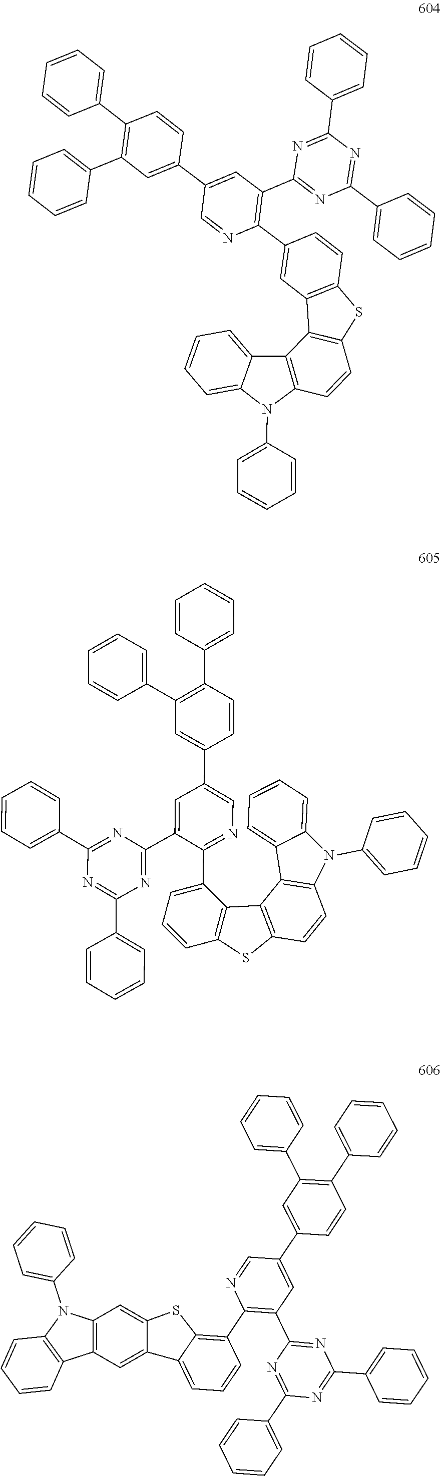 [SMILES: c1ccc(-c2nc(-c3ccccc3)nc(-c3cc(-c4ccc(-c5ccccc5)c(-c5ccccc5)c4)cnc3-c3ccc4sc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)cnc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c(cc34)c3ccccc3n5-c3ccccc3)n2)cc1]

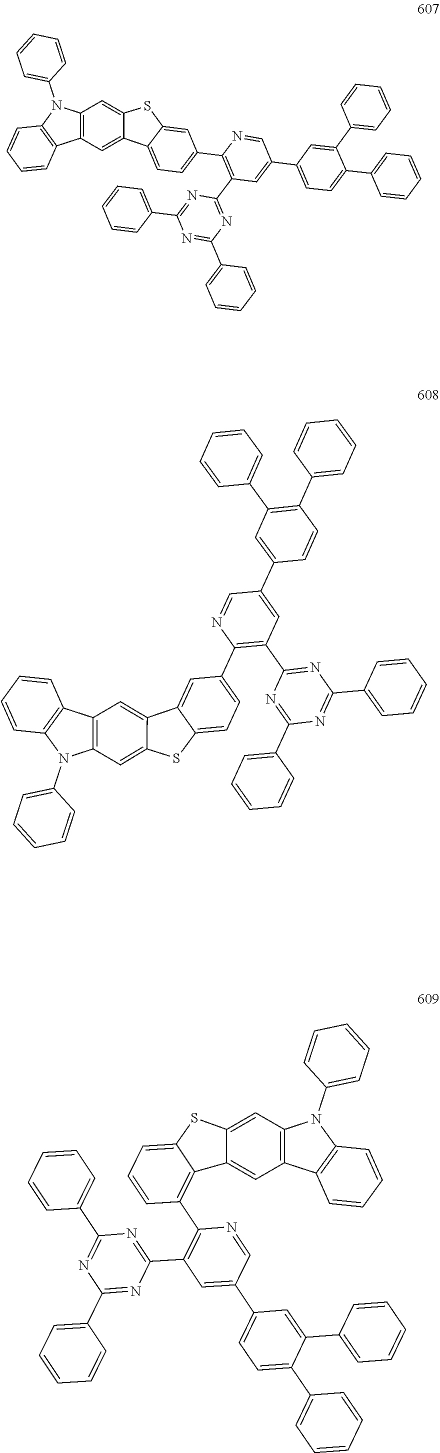 [SMILES: c1ccc(-c2nc(-c3ccccc3)nc(-c3cc(-c4ccc(-c5ccccc5)c(-c5ccccc5)c4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4sc5cc6c(cc5c34)c3ccccc3n6-c3ccccc3)n2)cc1]

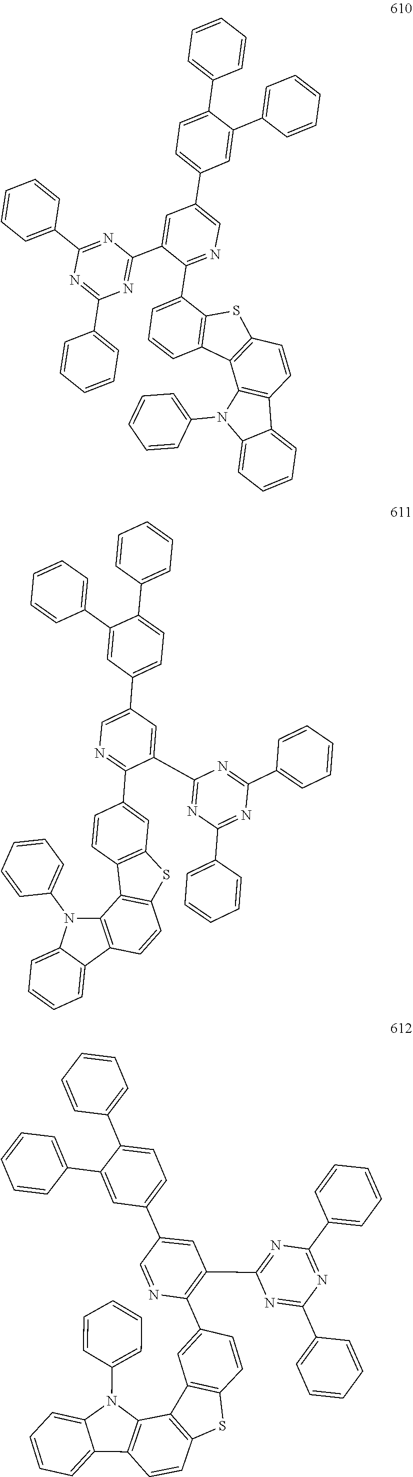 [SMILES: c1ccc(-c2nc(-c3ccccc3)nc(-c3cc(-c4ccc(-c5ccccc5)c(-c5ccccc5)c4)cnc3-c3cccc4c3sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4sc5ccc6c7ccccc7n(-c7ccccc7)c6c5c4c3)n2)cc1]

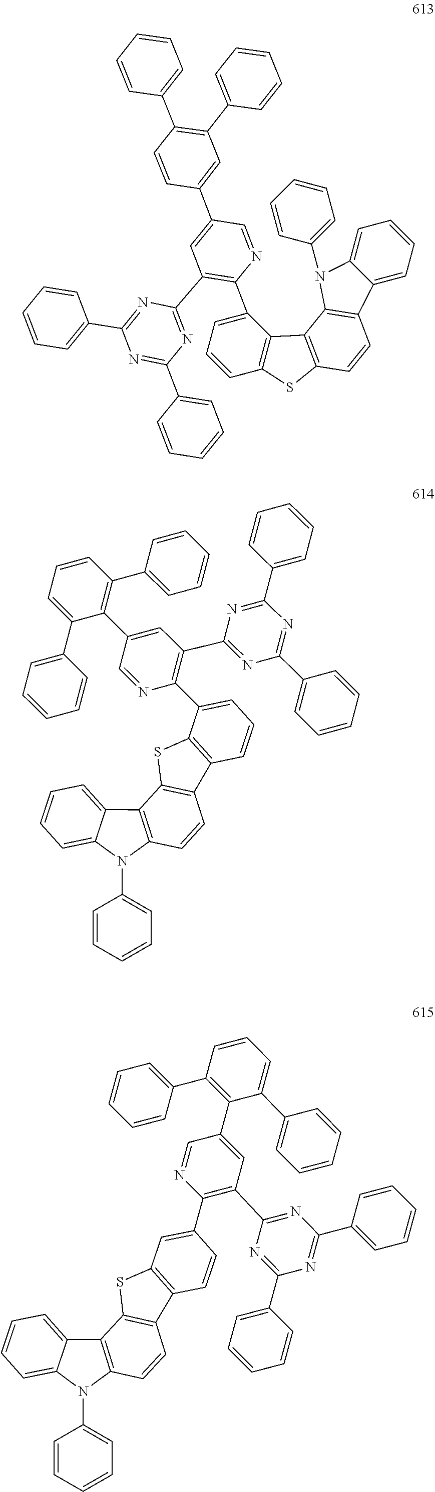 [SMILES: c1ccc(-c2nc(-c3ccccc3)nc(-c3cc(-c4ccc(-c5ccccc5)c(-c5ccccc5)c4)cnc3-c3cccc4sc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c3c3ccccc3n4-c3ccccc3)n2)cc1]

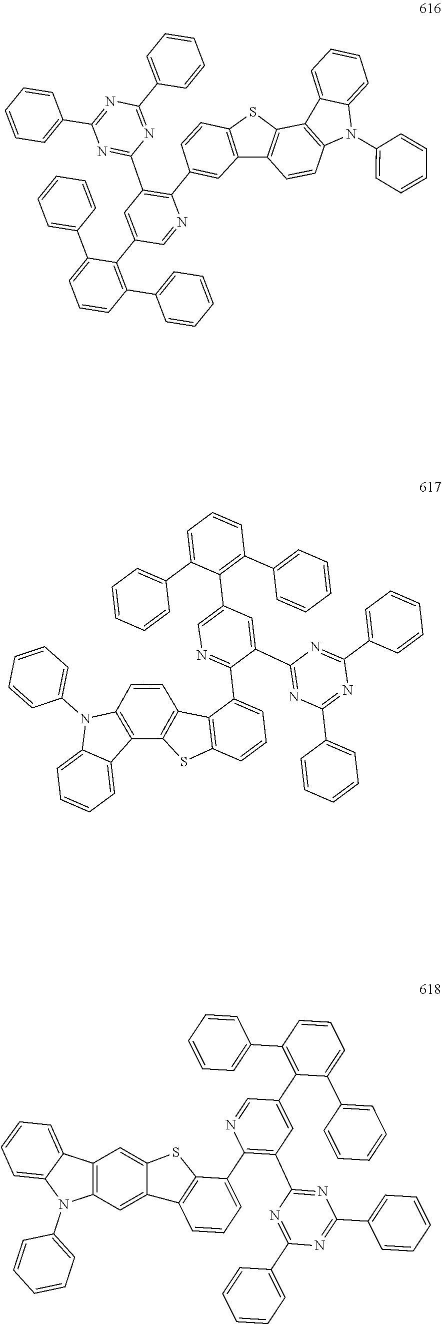 [SMILES: c1ccc(-c2nc(-c3ccccc3)nc(-c3cc(-c4c(-c5ccccc5)cccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)cnc3-c3cccc4c3sc3cc5c6ccccc6n(-c6ccccc6)c5cc34)n2)cc1]

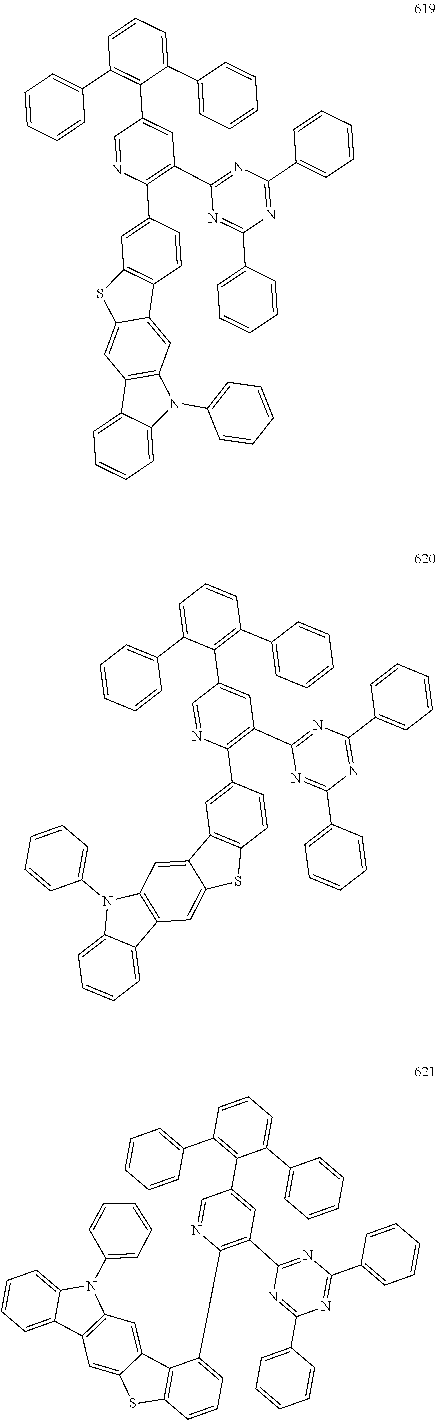 [SMILES: c1ccc(-c2nc(-c3ccccc3)nc(-c3cc(-c4c(-c5ccccc5)cccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4sc5cc6c7ccccc7n(-c7ccccc7)c6cc5c34)n2)cc1]

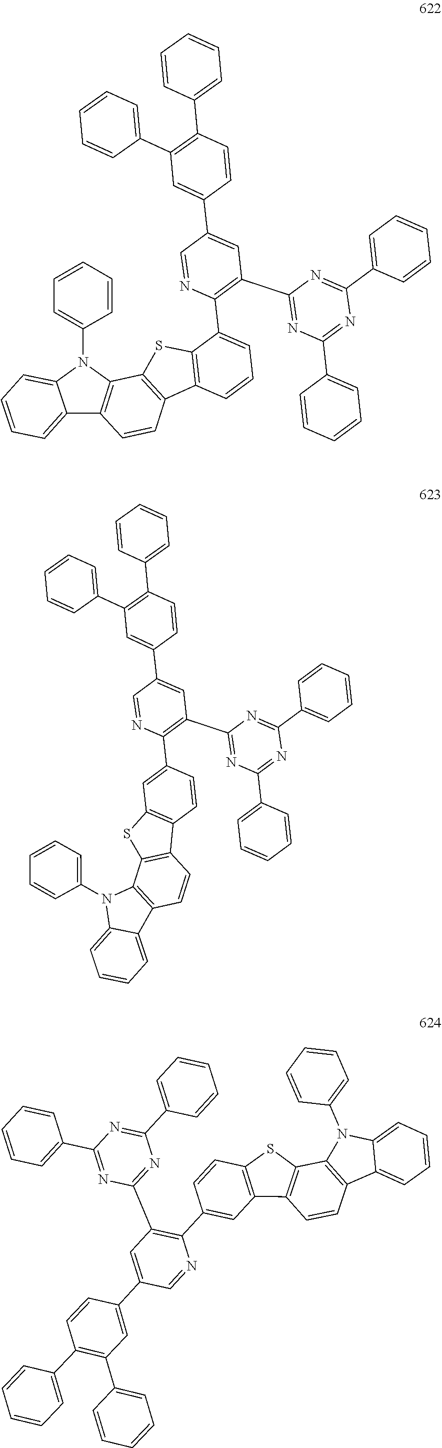 [SMILES: c1ccc(-c2nc(-c3ccccc3)nc(-c3cc(-c4ccc(-c5ccccc5)c(-c5ccccc5)c4)cnc3-c3cccc4c3sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4sc5c(ccc6c7ccccc7n(-c7ccccc7)c65)c4c3)n2)cc1]

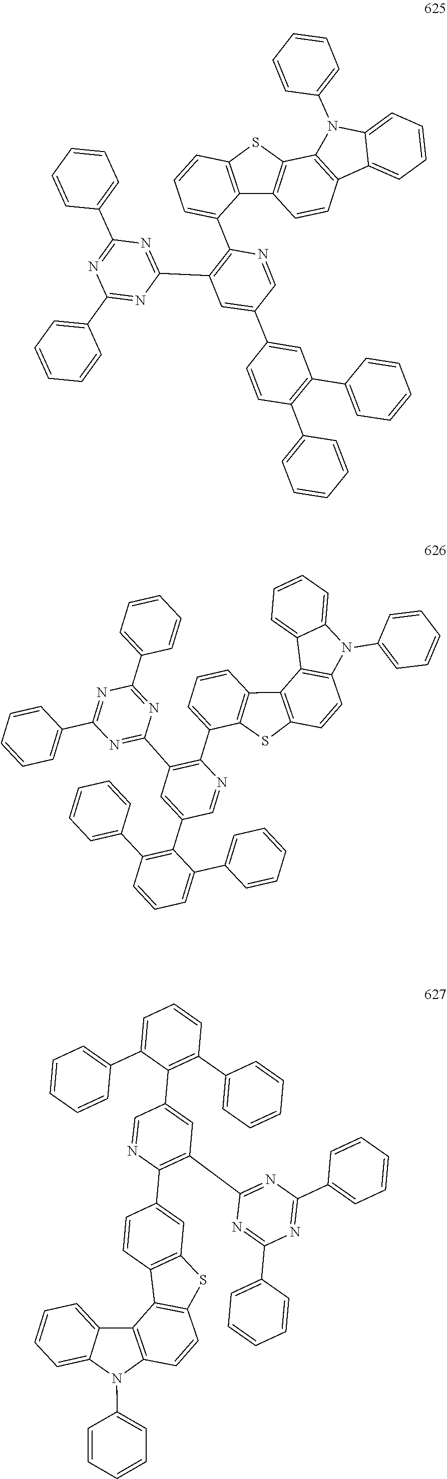 [SMILES: c1ccc(-c2nc(-c3ccccc3)nc(-c3cc(-c4ccc(-c5ccccc5)c(-c5ccccc5)c4)cnc3-c3cccc4sc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)cnc3-c3ccc4c(c3)sc3ccc5c(c6ccccc6n5-c5ccccc5)c34)n2)cc1]

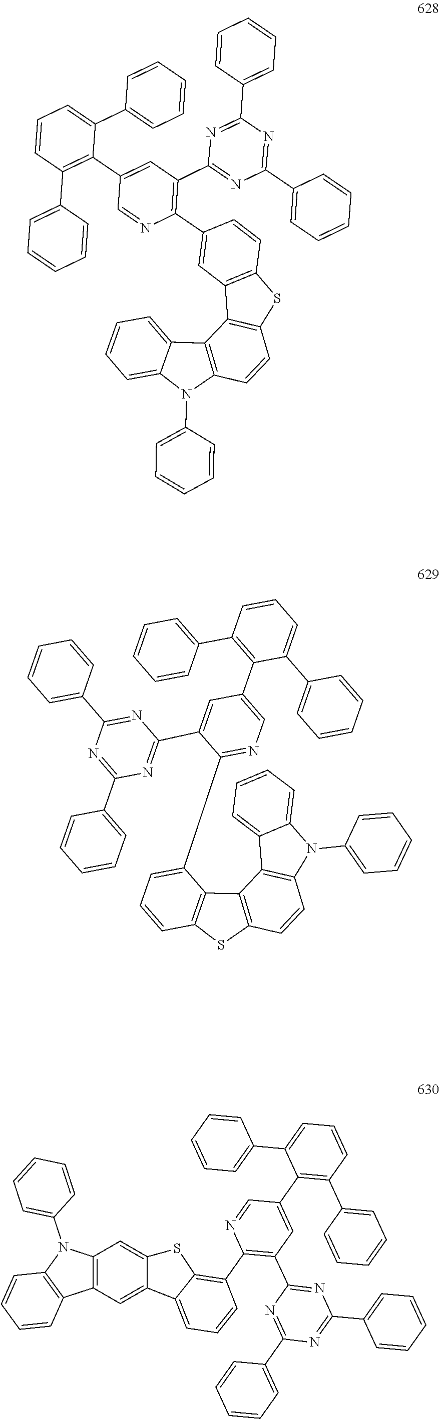 [SMILES: c1ccc(-c2nc(-c3ccccc3)nc(-c3cc(-c4c(-c5ccccc5)cccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)cnc3-c3cccc4c3sc3cc5c(cc34)c3ccccc3n5-c3ccccc3)n2)cc1]

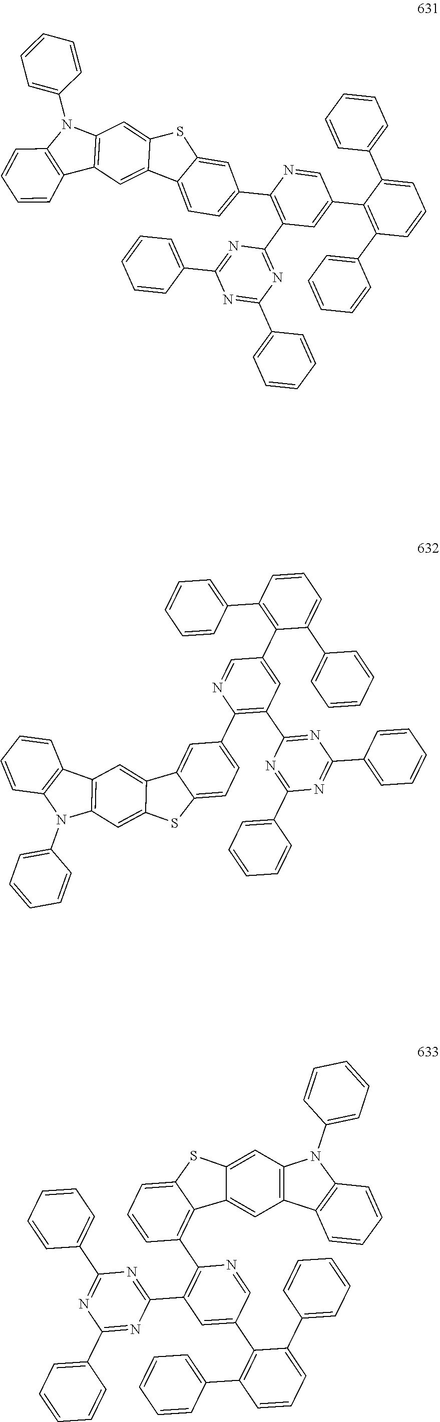 [SMILES: c1ccc(-c2nc(-c3ccccc3)nc(-c3cc(-c4c(-c5ccccc5)cccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4sc5cc6c(cc5c34)c3ccccc3n6-c3ccccc3)n2)cc1]

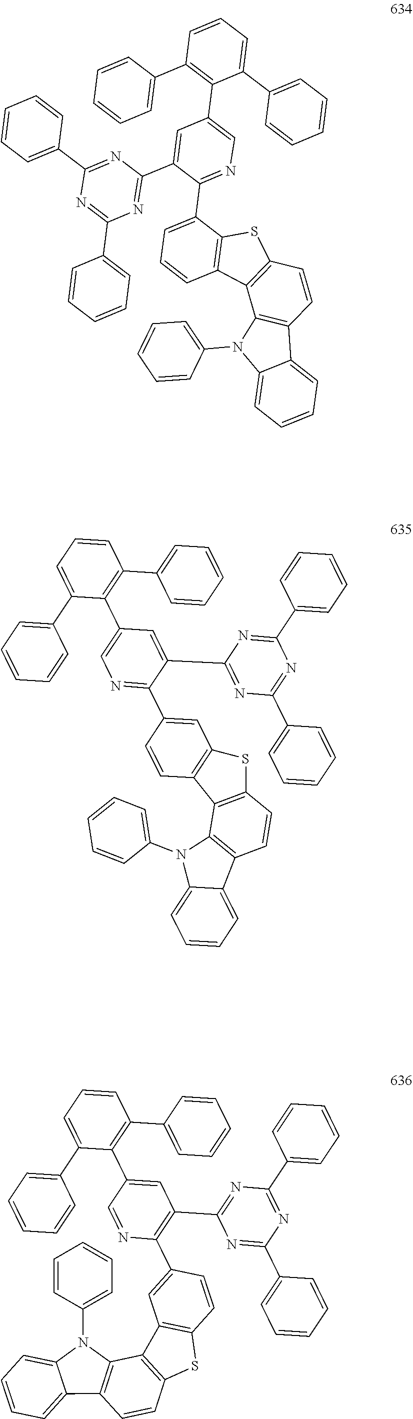 [SMILES: c1ccc(-c2nc(-c3ccccc3)nc(-c3cc(-c4c(-c5ccccc5)cccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4sc5ccc6c7ccccc7n(-c7ccccc7)c6c5c4c3)n2)cc1]

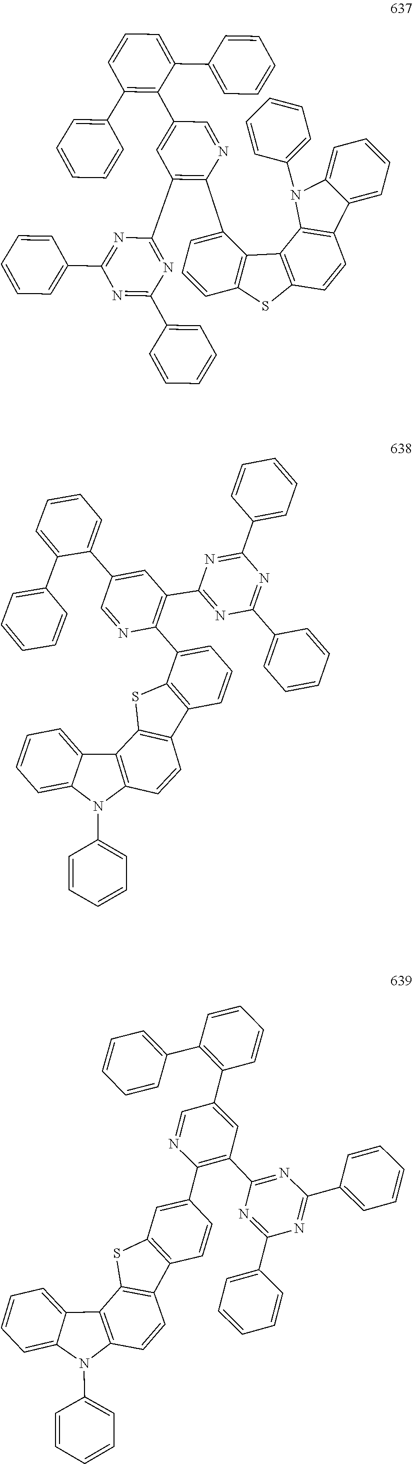 [SMILES: c1ccc(-c2nc(-c3ccccc3)nc(-c3cc(-c4c(-c5ccccc5)cccc4-c4ccccc4)cnc3-c3cccc4sc5ccc6c7ccccc7n(-c7ccccc7)c6c5c34)n2)cc1 ; c1ccc(-c2nc(-c3ccccc3)nc(-c3cc(-c4ccccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)sc3c4ccc4c3c3ccccc3n4-c3ccccc3)n2)cc1]

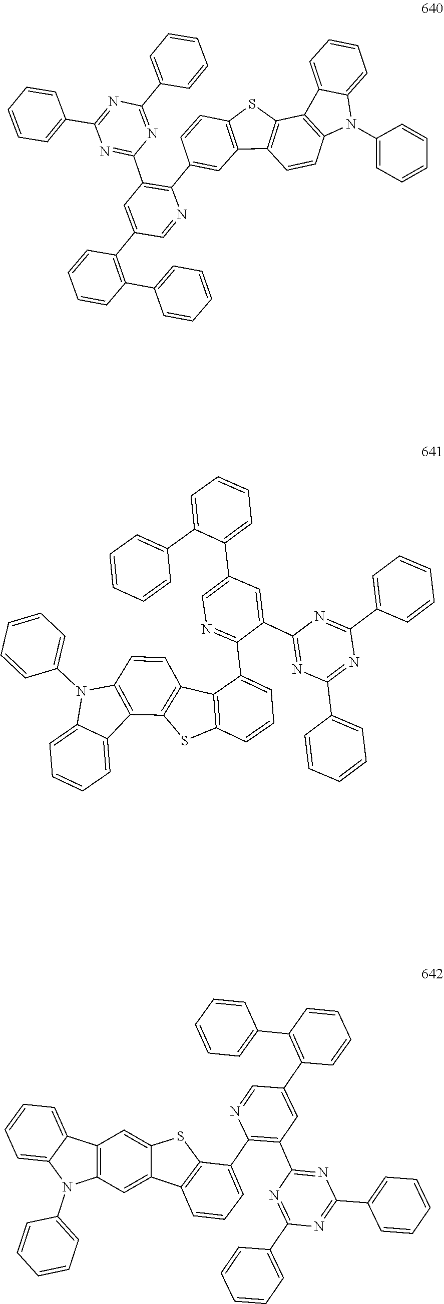 [SMILES: c1ccc(-c2nc(-c3ccccc3)nc(-c3cc(-c4ccccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4sc5c(ccc6c5c5ccccc5n6-c5ccccc5)c34)n2)cc1 ; c1ccc(-c2nc(-c3ccccc3)nc(-c3cc(-c4ccccc4-c4ccccc4)cnc3-c3cccc4c3sc3cc5c6ccccc6n(-c6ccccc6)c5cc34)n2)cc1]

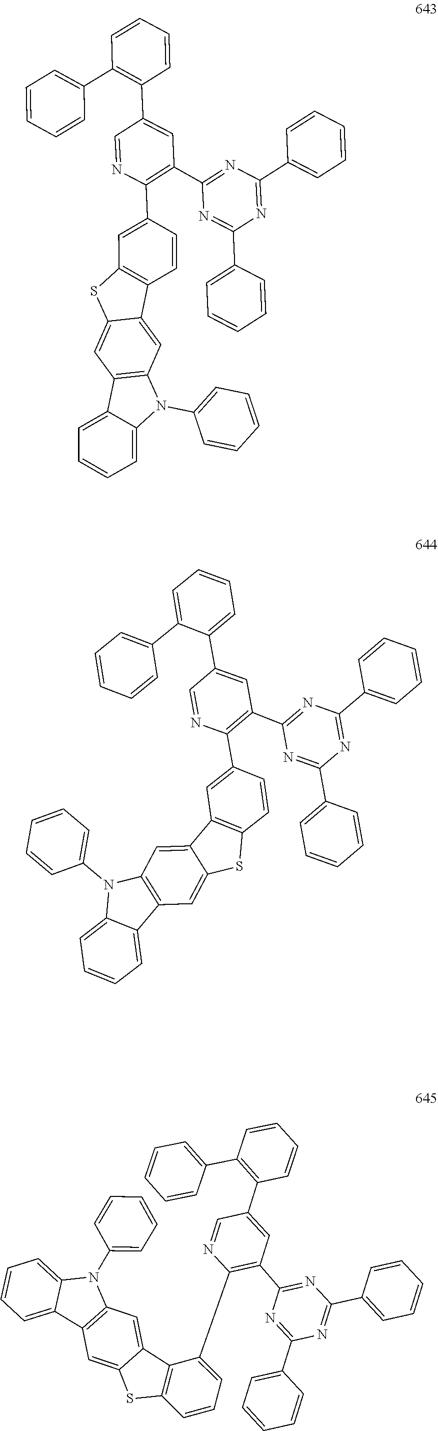 [SMILES: c1ccc(-c2nc(-c3ccccc3)nc(-c3cc(-c4ccccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4sc5cc6c7ccccc7n(-c7ccccc7)c6cc5c34)n2)cc1]

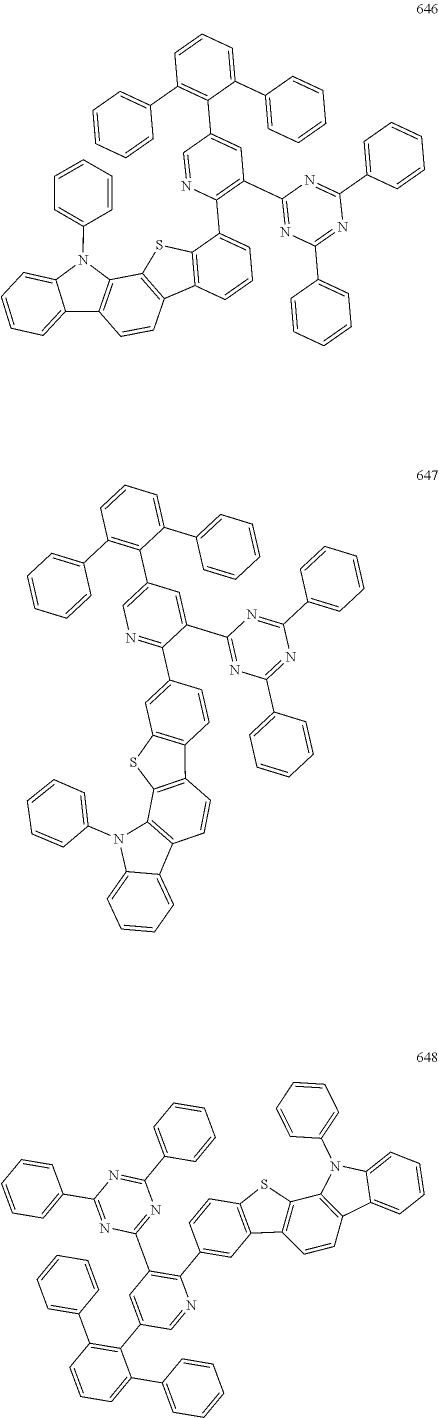 [SMILES: c1ccc(-c2nc(-c3ccccc3)nc(-c3cc(-c4c(-c5ccccc5)cccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4sc5c(ccc6c7ccccc7n(-c7ccccc7)c65)c4c3)n2)cc1]

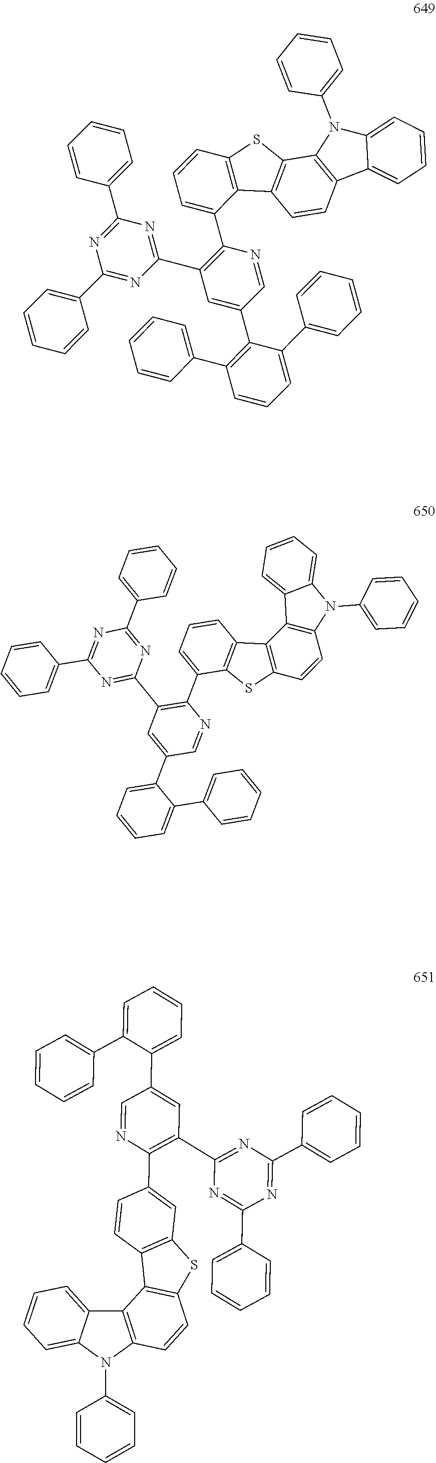 [SMILES: c1ccc(-c2nc(-c3ccccc3)nc(-c3cc(-c4c(-c5ccccc5)cccc4-c4ccccc4)cnc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)sc3ccc5c(c6ccccc6n5-c5ccccc5)c34)n2)cc1]

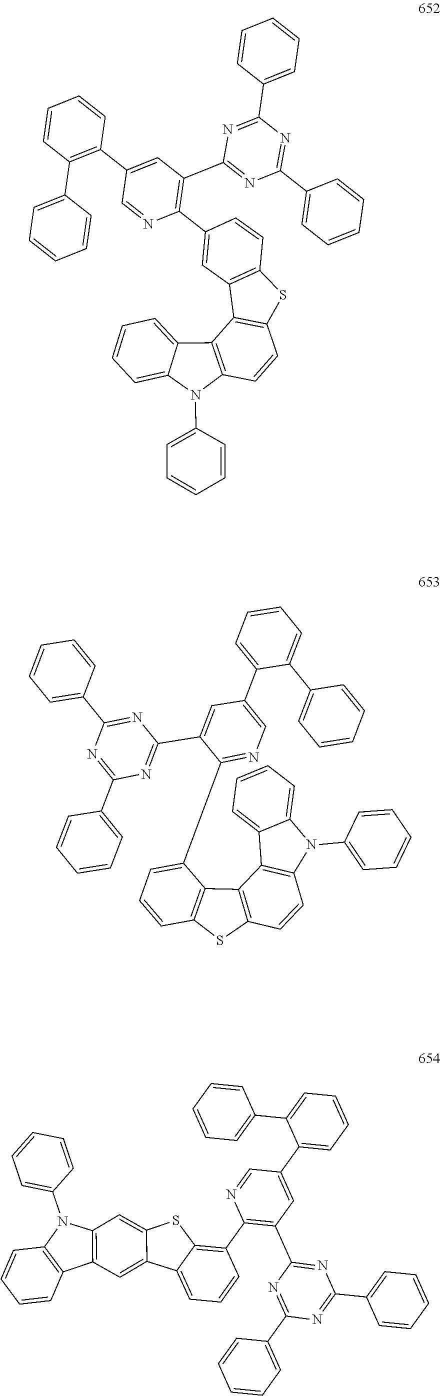 [SMILES: c1ccc(-c2nc(-c3ccccc3)nc(-c3cc(-c4ccccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3sc3cc5c(cc34)c3ccccc3n5-c3ccccc3)n2)cc1]

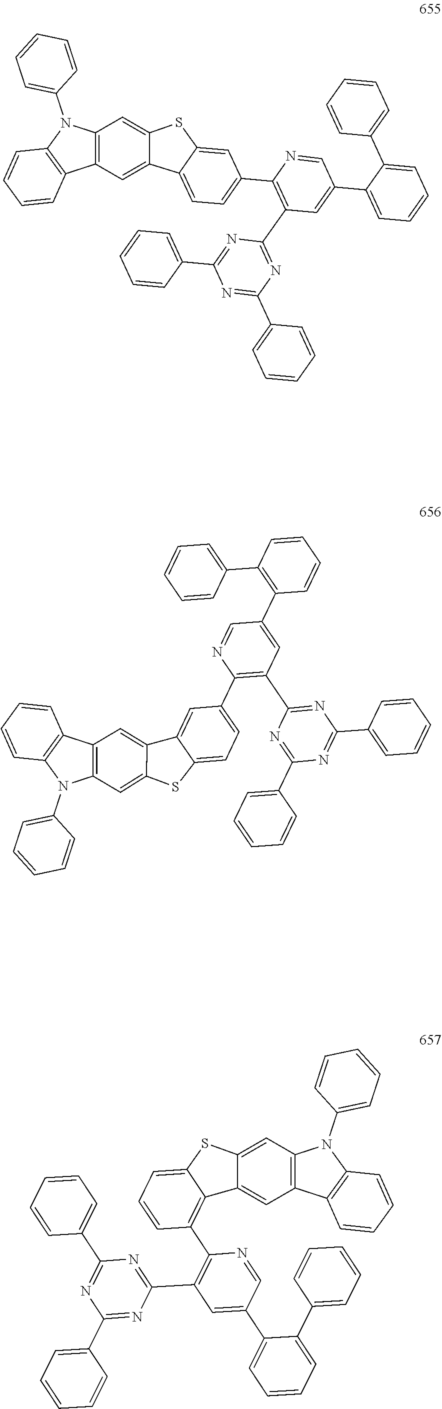 [SMILES: c1ccc(-c2nc(-c3ccccc3)nc(-c3cc(-c4ccccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4sc5cc6c(cc5c34)c3ccccc3n6-c3ccccc3)n2)cc1]

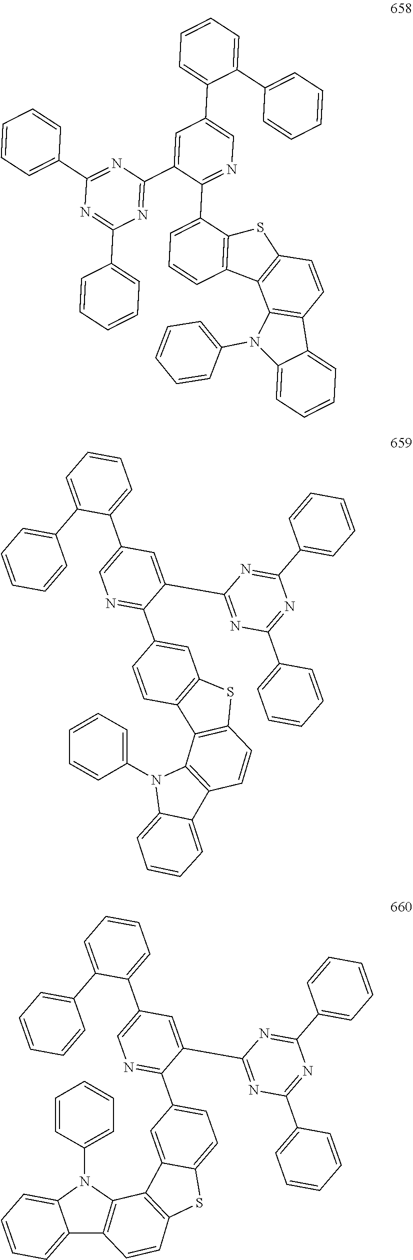 [SMILES: c1ccc(-c2nc(-c3ccccc3)nc(-c3cc(-c4ccccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4sc5ccc6c7ccccc7n(-c7ccccc7)c6c5c4c3)n2)cc1]

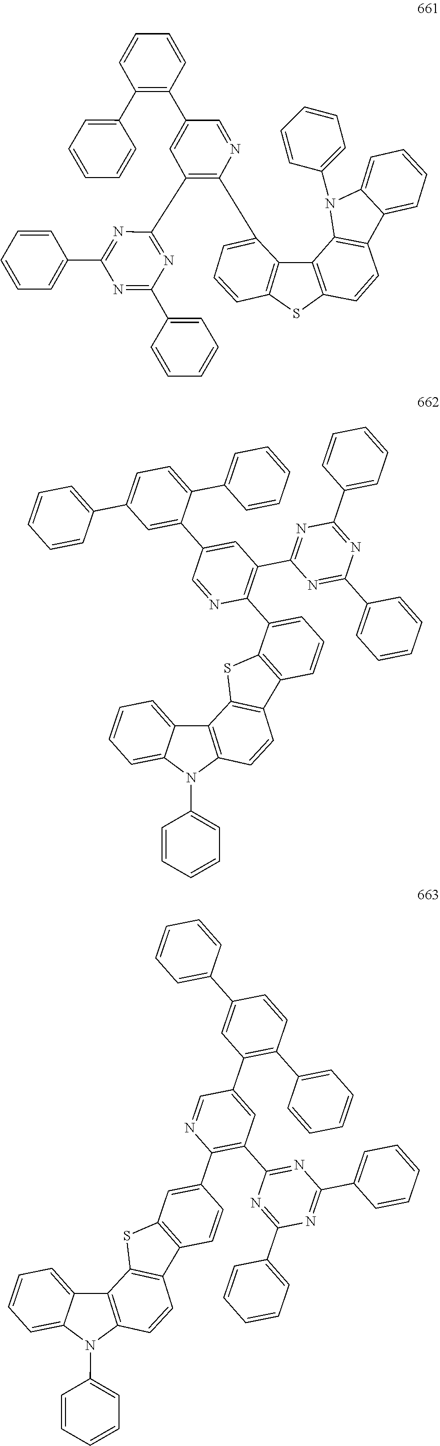

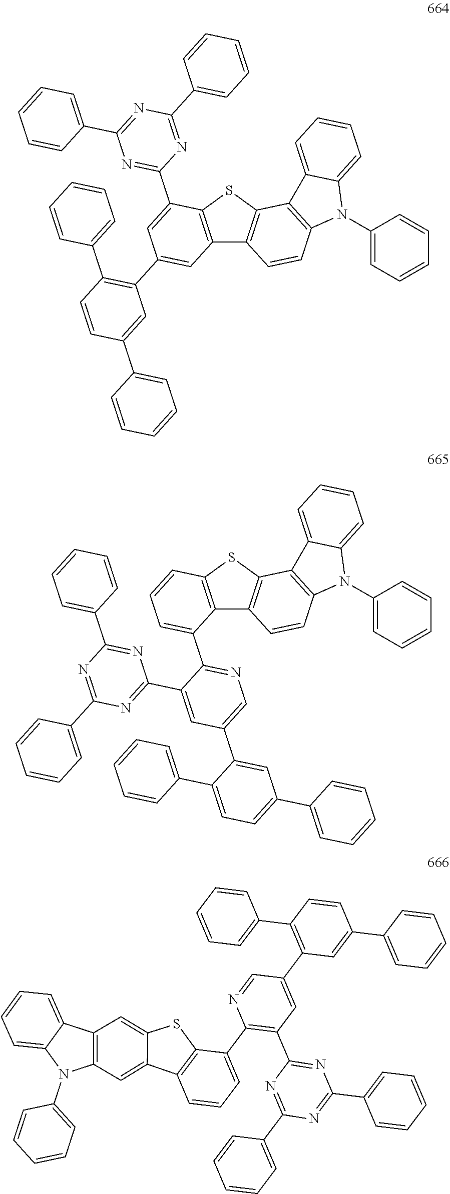 [SMILES: c1ccc(-c2ccc(-c3ccccc3)c(-c3cc(-c4nc(-c5ccccc5)nc(-c5ccccc5)n4)c4sc5c(ccc6c5c5ccccc5n6-c5ccccc5)c4c3)c2)cc1 ; c1ccc(-c2ccc(-c3ccccc3)c(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4cc6c7ccccc7n(-c7ccccc7)c6cc45)c(-c4nc(-c5ccccc5)nc(-c5ccccc5)n4)c3)c2)cc1]

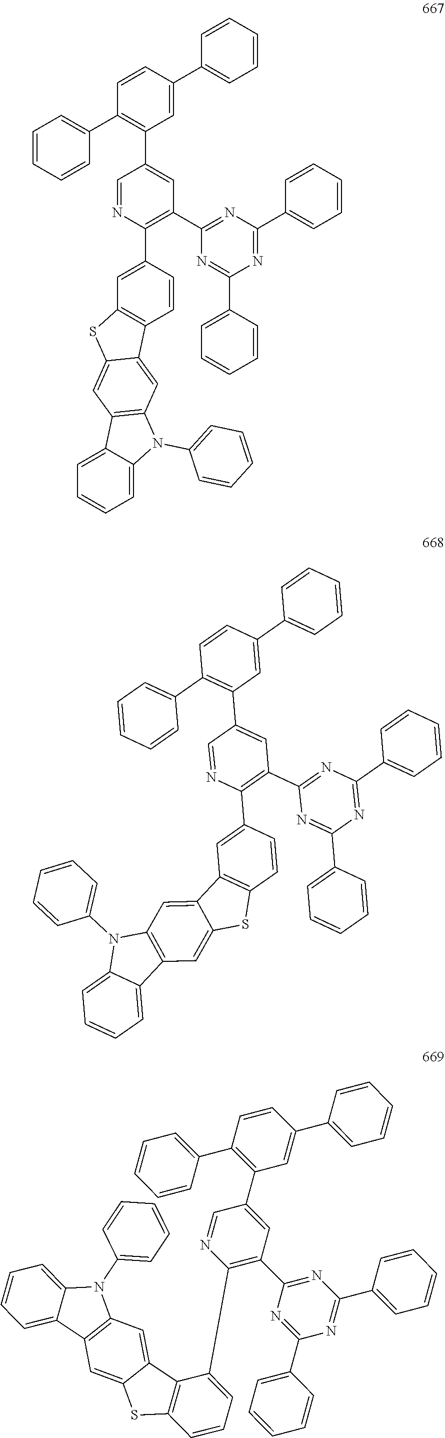 [SMILES: c1ccc(-c2ccc(-c3ccccc3)c(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6cc7c8ccccc8n(-c8ccccc8)c7cc6c45)c(-c4nc(-c5ccccc5)nc(-c5ccccc5)n4)c3)c2)cc1]

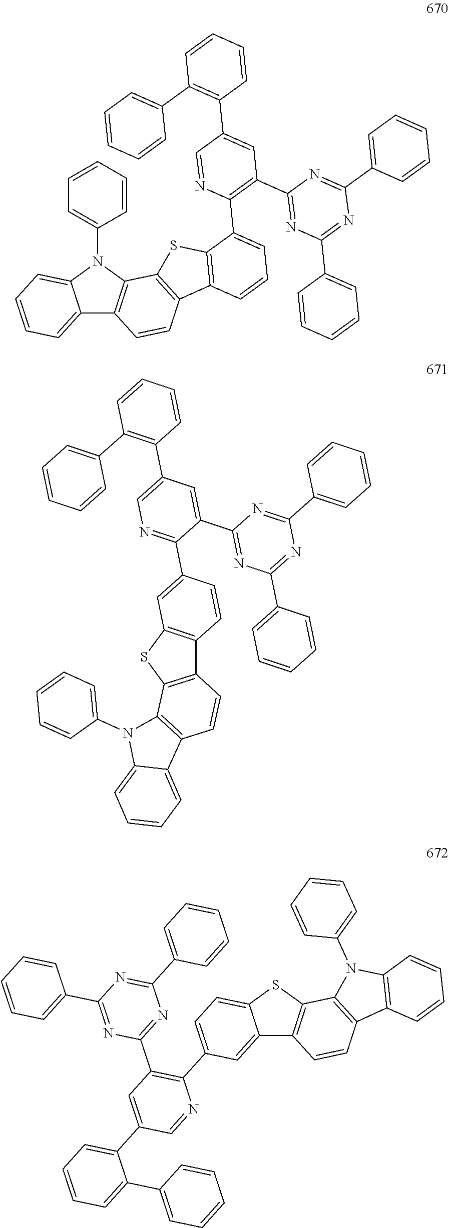

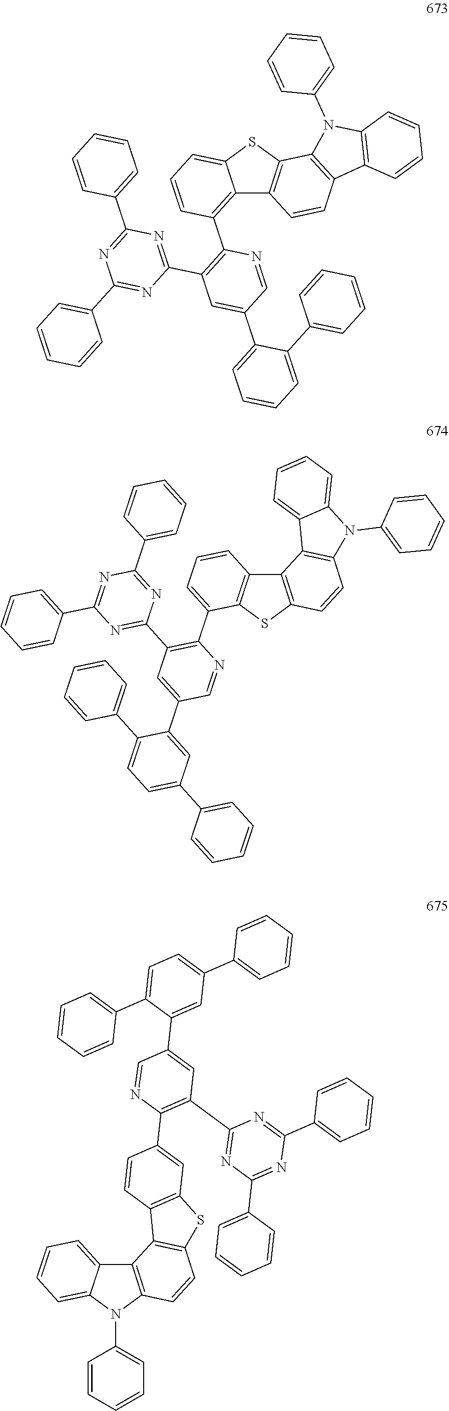 [SMILES: c1ccc(-c2nc(-c3ccccc3)nc(-c3cc(-c4ccccc4-c4ccccc4)cnc3-c3cccc4sc5c(ccc6c7ccccc7n(-c7ccccc7)c65)c34)n2)cc1 ; c1ccc(-c2ccc(-c3ccccc3)c(-c3cnc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1]

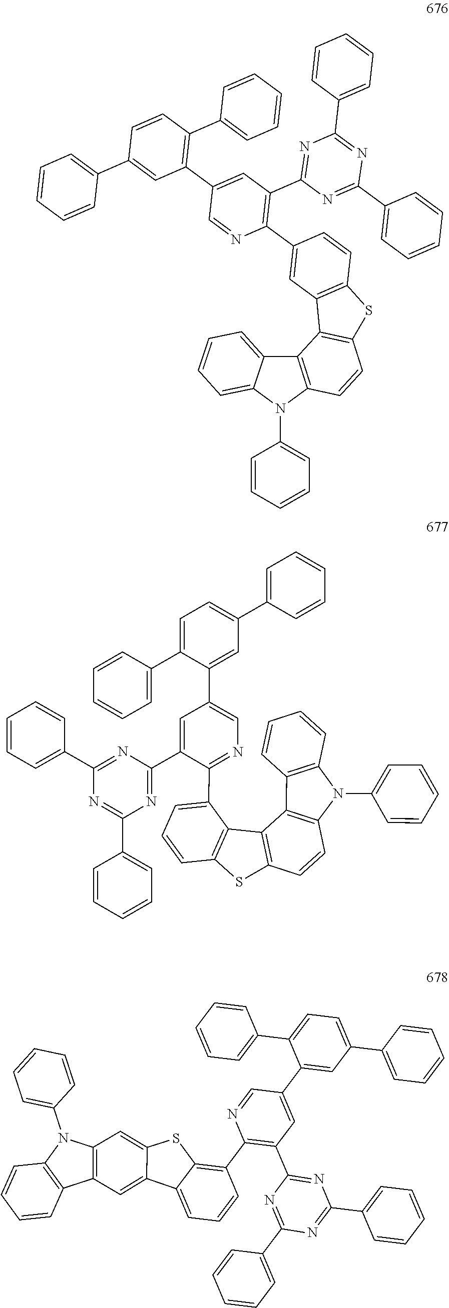

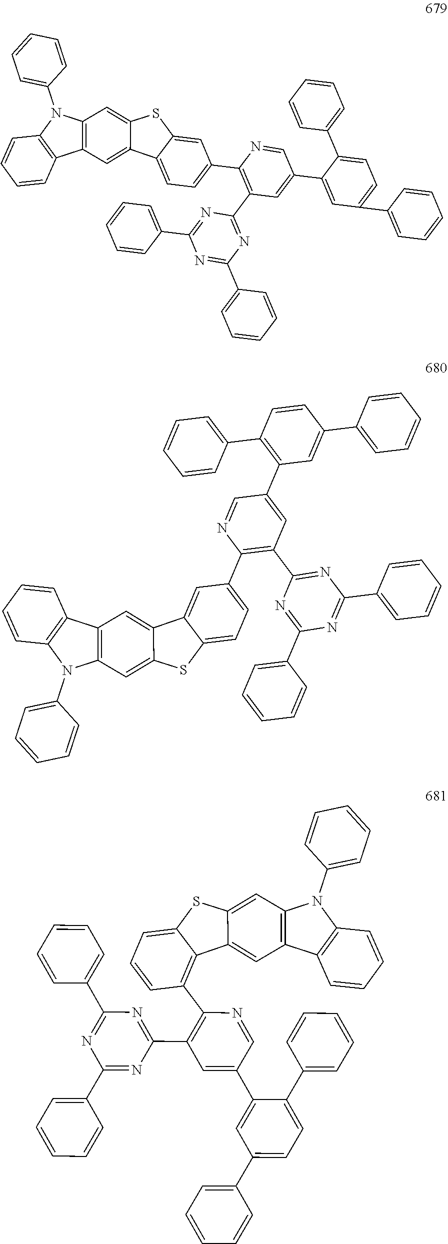 [SMILES: c1ccc(-c2ccc(-c3ccccc3)c(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5sc6cc7c(cc6c45)c4ccccc4n7-c4ccccc4)c(-c4nc(-c5ccccc5)nc(-c5ccccc5)n4)c3)c2)cc1]

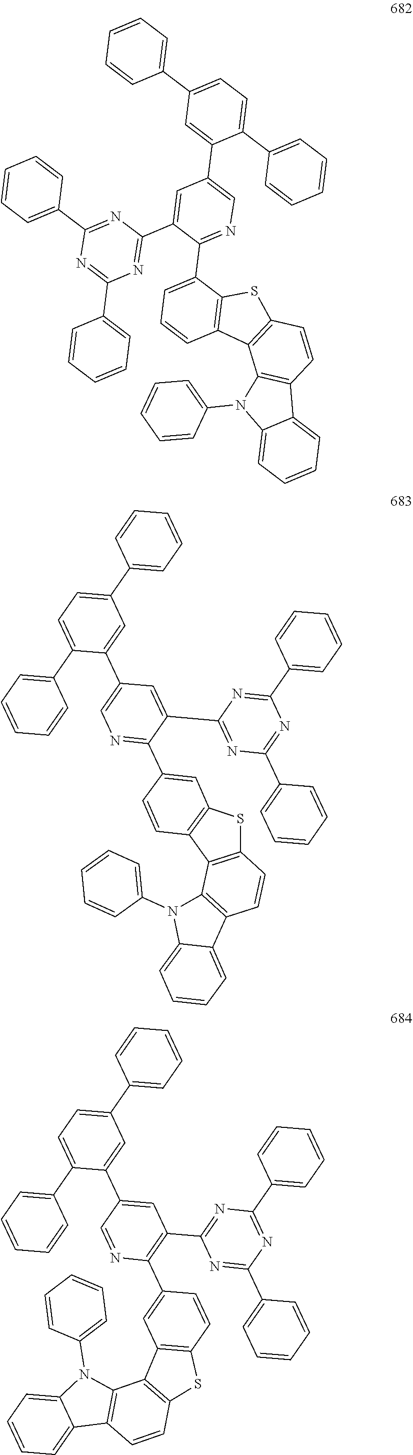

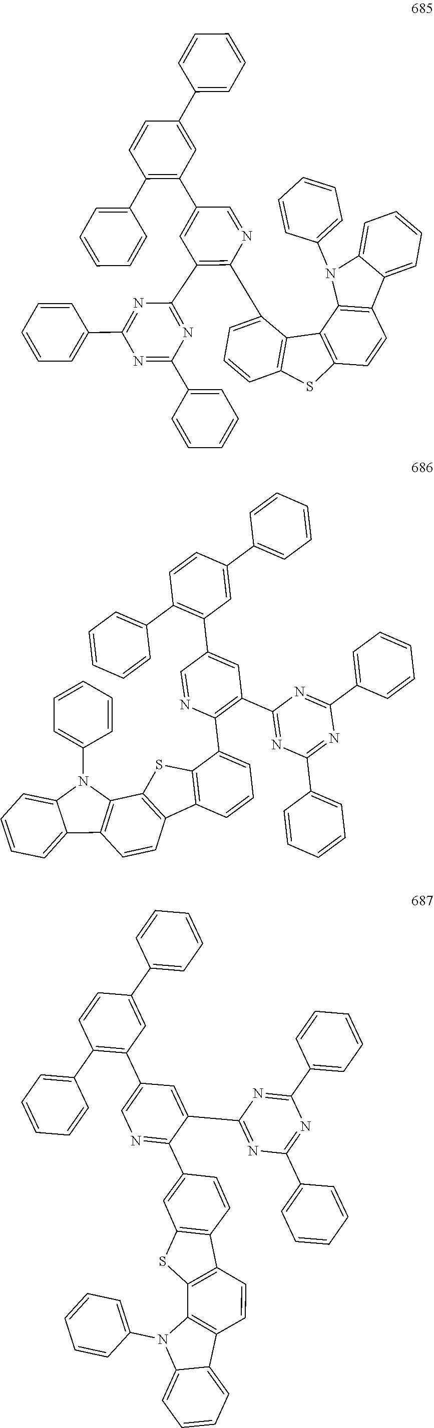 [SMILES: c1ccc(-c2ccc(-c3ccccc3)c(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1]

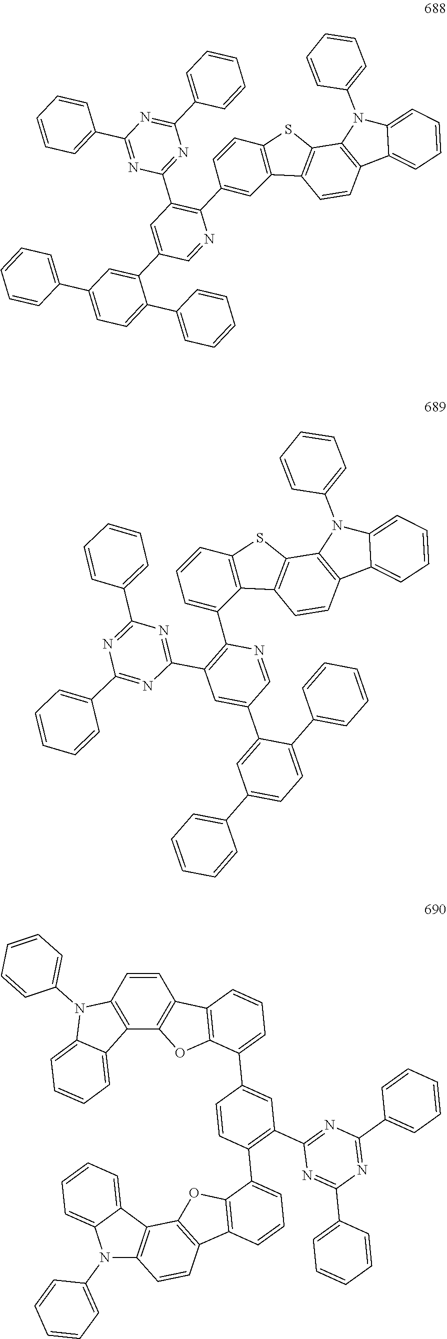 [SMILES: c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4cccc5c4oc4c5ccc5c4c4ccccc4n5-c4ccccc4)ccc3-c3cccc4c3oc3c4ccc4c3c3ccccc3n4-c3ccccc3)n2)cc1]

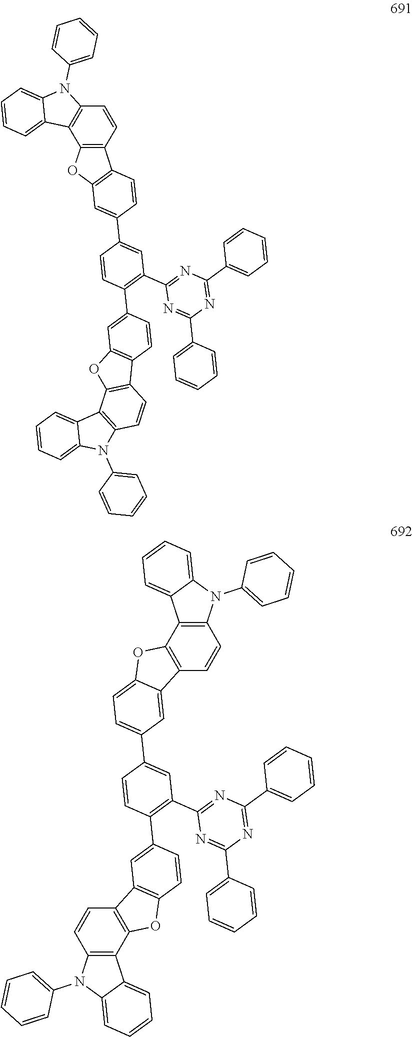 [SMILES: c1ccc(-c2nc(-c3ccccc3)nc(-c3cc(-c4ccc5c(c4)oc4c5ccc5c4c4ccccc4n5-c4ccccc4)ccc3-c3ccc4c(c3)oc3c4ccc4c3c3ccccc3n4-c3ccccc3)n2)cc1 ; c1ccc(-c2nc(-c3ccccc3)nc(-c3cc(-c4ccc5oc6c(ccc7c6c6ccccc6n7-c6ccccc6)c5c4)ccc3-c3ccc4oc5c(ccc6c5c5ccccc5n6-c5ccccc5)c4c3)n2)cc1]

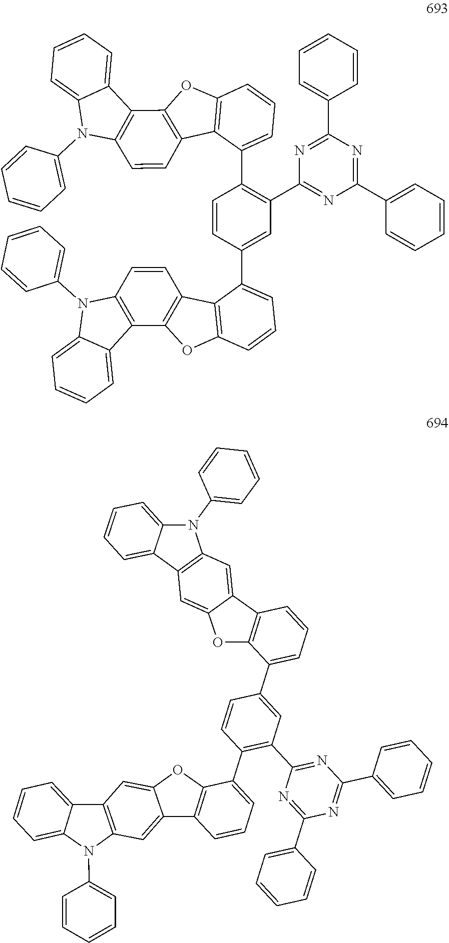 [SMILES: c1ccc(-c2nc(-c3ccccc3)nc(-c3cc(-c4cccc5oc6c(ccc7c6c6ccccc6n7-c6ccccc6)c45)ccc3-c3cccc4oc5c(ccc6c5c5ccccc5n6-c5ccccc5)c34)n2)cc1 ; c1ccc(-c2nc(-c3ccccc3)nc(-c3cc(-c4cccc5c4oc4cc6c7ccccc7n(-c7ccccc7)c6cc45)ccc3-c3cccc4c3oc3cc5c6ccccc6n(-c6ccccc6)c5cc34)n2)cc1]

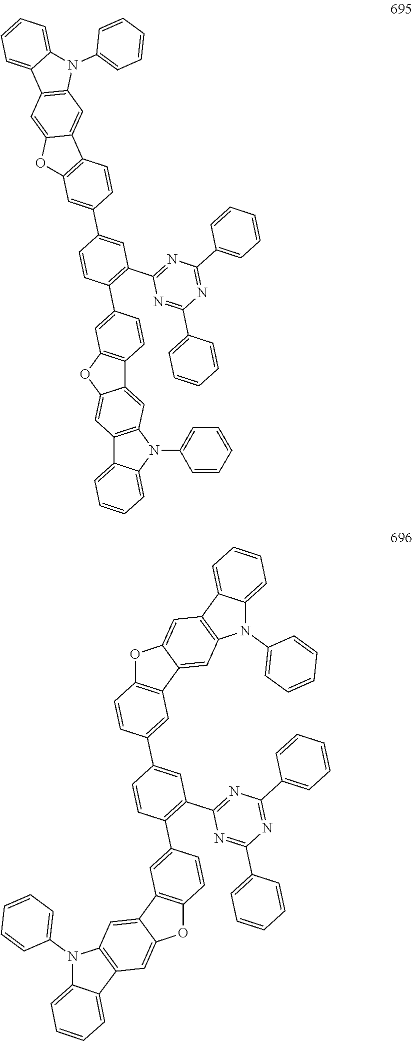 [SMILES: c1ccc(-c2nc(-c3ccccc3)nc(-c3cc(-c4ccc5c(c4)oc4cc6c7ccccc7n(-c7ccccc7)c6cc45)ccc3-c3ccc4c(c3)oc3cc5c6ccccc6n(-c6ccccc6)c5cc34)n2)cc1 ; c1ccc(-c2nc(-c3ccccc3)nc(-c3cc(-c4ccc5oc6cc7c8ccccc8n(-c8ccccc8)c7cc6c5c4)ccc3-c3ccc4oc5cc6c7ccccc7n(-c7ccccc7)c6cc5c4c3)n2)cc1]

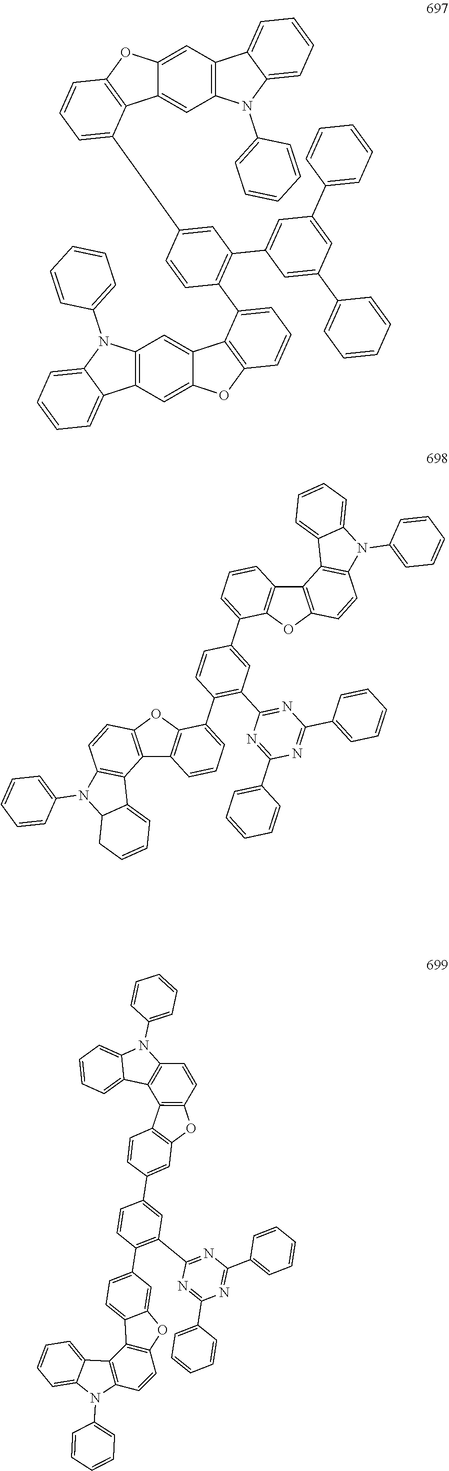 [SMILES: c1ccc(-c2cc(-c3ccccc3)cc(-c3cc(-c4cccc5oc6cc7c8ccccc8n(-c8ccccc8)c7cc6c45)ccc3-c3cccc4oc5cc6c7ccccc7n(-c7ccccc7)c6cc5c34)c2)cc1 ; C1=CCC2C(=C1)c1c(ccc3oc4c(-c5ccc(-c6cccc7c6oc6ccc8c(c9ccccc9n8-c8ccccc8)c67)cc5-c5nc(-c6ccccc6)nc(-c6ccccc6)n5)cccc4c13)N2c1ccccc1 ; c1ccc(-c2nc(-c3ccccc3)nc(-c3cc(-c4ccc5c(c4)oc4ccc6c(c7ccccc7n6-c6ccccc6)c45)ccc3-c3ccc4c(c3)oc3ccc5c(c6ccccc6n5-c5ccccc5)c34)n2)cc1]

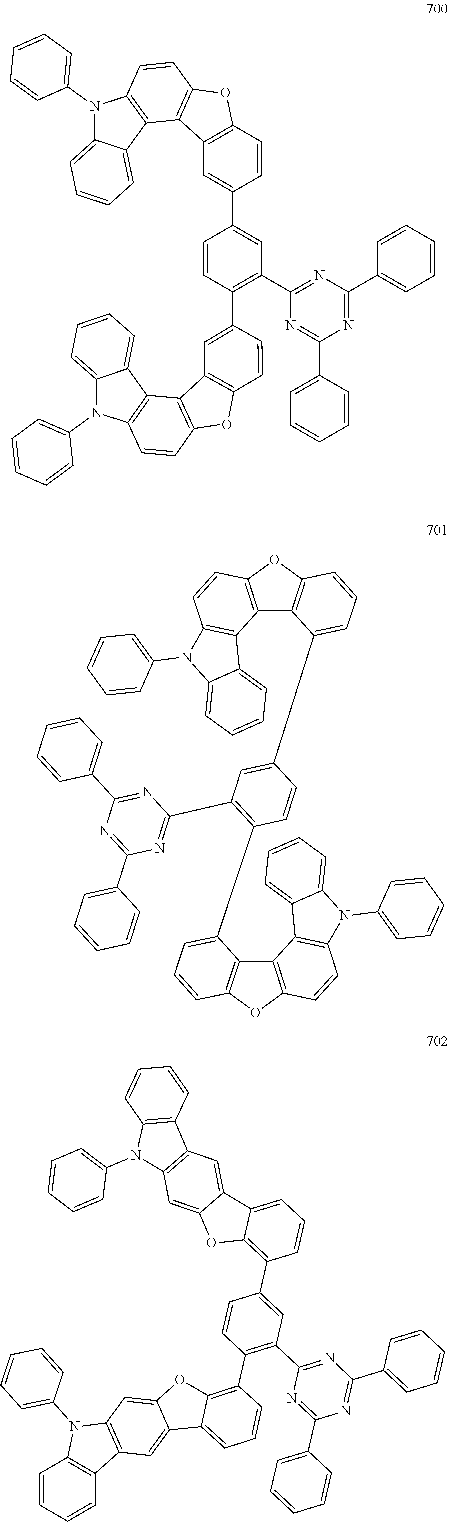 [SMILES: c1ccc(-c2nc(-c3ccccc3)nc(-c3cc(-c4ccc5oc6ccc7c(c8ccccc8n7-c7ccccc7)c6c5c4)ccc3-c3ccc4oc5ccc6c(c7ccccc7n6-c6ccccc6)c5c4c3)n2)cc1 ; c1ccc(-c2nc(-c3ccccc3)nc(-c3cc(-c4cccc5oc6ccc7c(c8ccccc8n7-c7ccccc7)c6c45)ccc3-c3cccc4oc5ccc6c(c7ccccc7n6-c6ccccc6)c5c34)n2)cc1 ; c1ccc(-c2nc(-c3ccccc3)nc(-c3cc(-c4cccc5c4oc4cc6c(cc45)c4ccccc4n6-c4ccccc4)ccc3-c3cccc4c3oc3cc5c(cc34)c3ccccc3n5-c3ccccc3)n2)cc1]

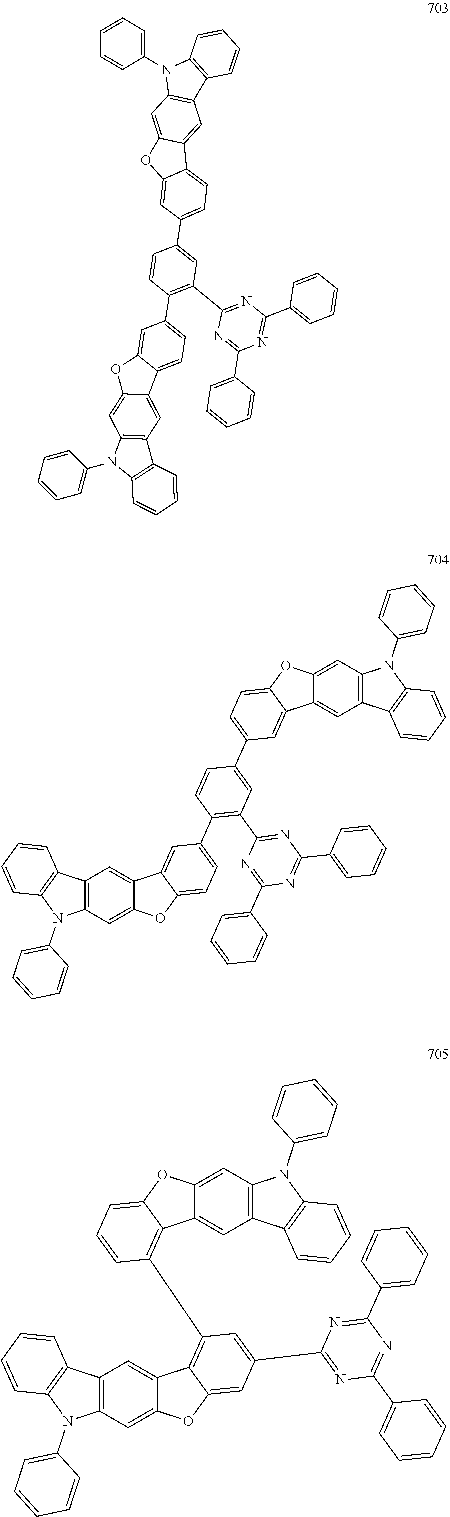 [SMILES: c1ccc(-c2nc(-c3ccccc3)nc(-c3cc(-c4ccc5c(c4)oc4cc6c(cc45)c4ccccc4n6-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccc5oc6cc7c(cc6c5c4)c4ccccc4n7-c4ccccc4)ccc3-c3ccc4oc5cc6c(cc5c4c3)c3ccccc3n6-c3ccccc3)n2)cc1 ; c1ccc(-c2nc(-c3ccccc3)nc(-c3cc(-c4cccc5oc6cc7c(cc6c45)c4ccccc4n7-c4ccccc4)c4c(c3)oc3cc5c(cc34)c3ccccc3n5-c3ccccc3)n2)cc1]

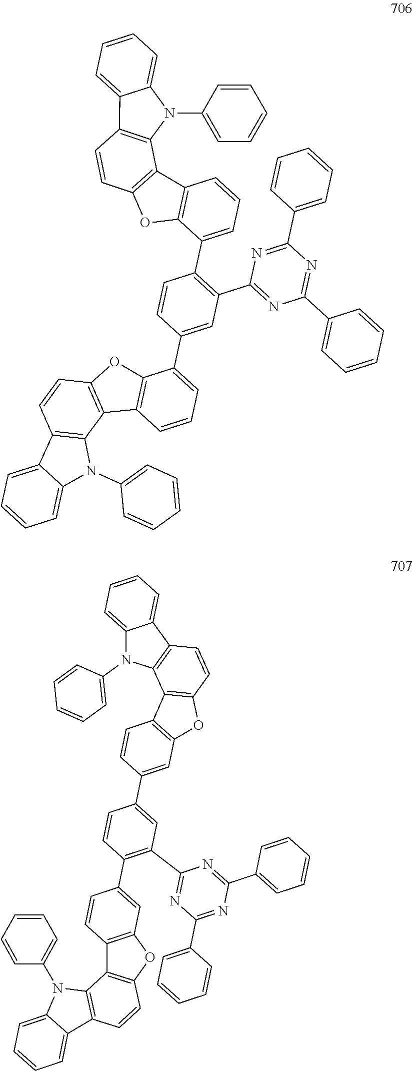 [SMILES: c1ccc(-c2nc(-c3ccccc3)nc(-c3cc(-c4cccc5c4oc4ccc6c7ccccc7n(-c7ccccc7)c6c45)ccc3-c3cccc4c3oc3ccc5c6ccccc6n(-c6ccccc6)c5c34)n2)cc1 ; c1ccc(-c2nc(-c3ccccc3)nc(-c3cc(-c4ccc5c(c4)oc4ccc6c7ccccc7n(-c7ccccc7)c6c45)ccc3-c3ccc4c(c3)oc3ccc5c6ccccc6n(-c6ccccc6)c5c34)n2)cc1]

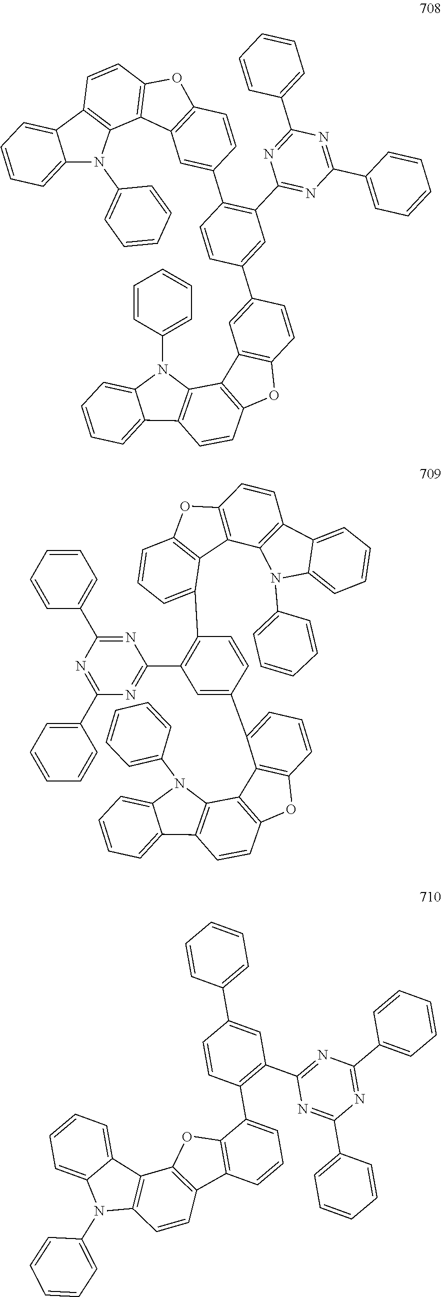

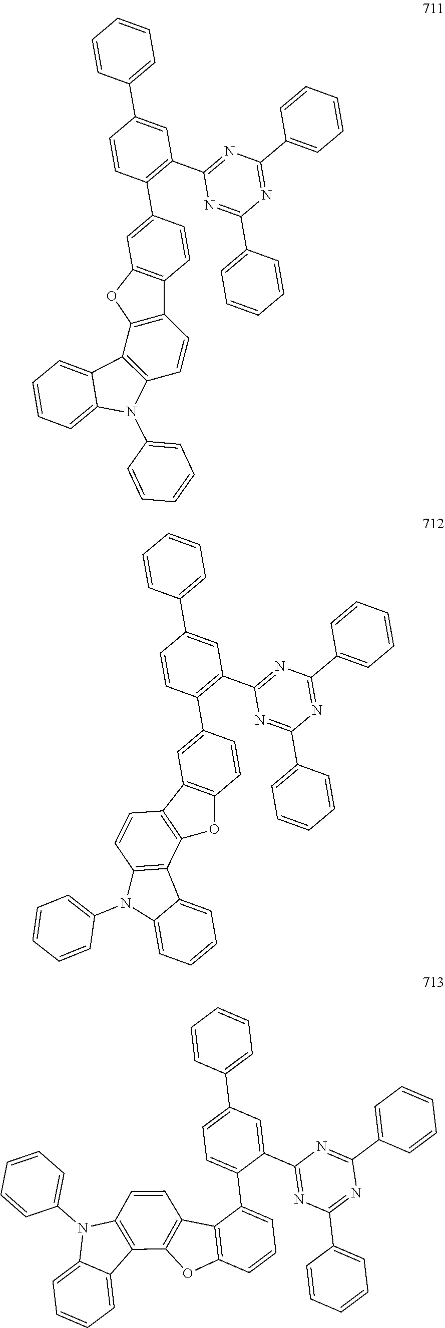

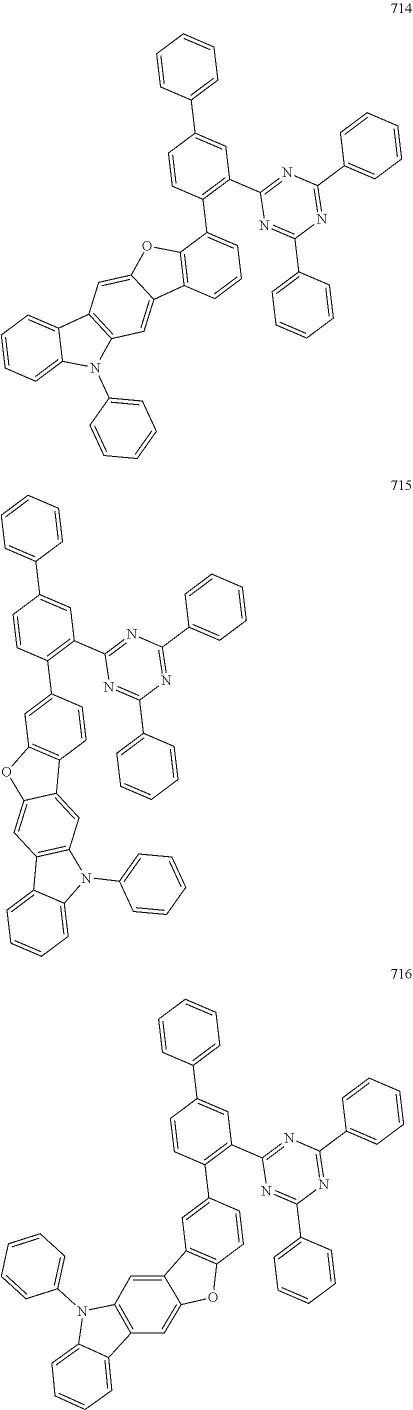 [SMILES: c1ccc(-c2ccc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4c(c3)oc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4oc5cc6c7ccccc7n(-c7ccccc7)c6cc5c4c3)c(-c3nc(-c4ccccc4)nc(-c4ccccc4)n3)c2)cc1]

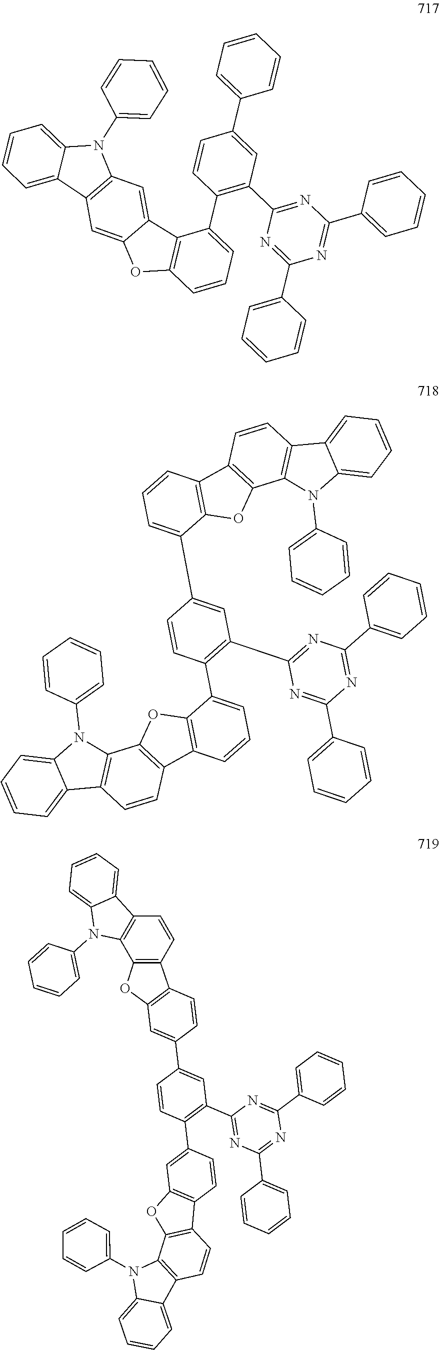 [SMILES: c1ccc(-c2ccc(-c3cccc4oc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4oc4c5ccc5c6ccccc6n(-c6ccccc6)c54)ccc3-c3cccc4c3oc3c4ccc4c5ccccc5n(-c5ccccc5)c43)n2)cc1 ; c1ccc(-c2nc(-c3ccccc3)nc(-c3cc(-c4ccc5c(c4)oc4c5ccc5c6ccccc6n(-c6ccccc6)c54)ccc3-c3ccc4c(c3)oc3c4ccc4c5ccccc5n(-c5ccccc5)c43)n2)cc1]

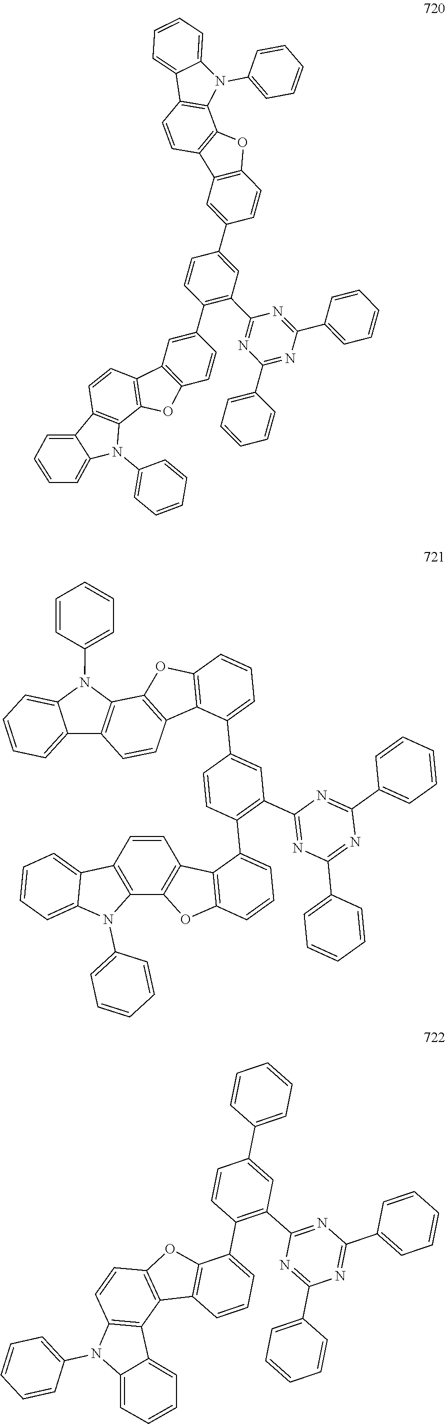 [SMILES: c1ccc(-c2nc(-c3ccccc3)nc(-c3cc(-c4ccc5oc6c(ccc7c8ccccc8n(-c8ccccc8)c76)c5c4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4cccc5oc6c(ccc7c8ccccc8n(-c8ccccc8)c76)c45)ccc3-c3cccc4oc5c(ccc6c7ccccc7n(-c7ccccc7)c65)c34)n2)cc1 ; c1ccc(-c2ccc(-c3cccc4c3oc3ccc5c(c6ccccc6n5-c5ccccc5)c34)c(-c3nc(-c4ccccc4)nc(-c4ccccc4)n3)c2)cc1]

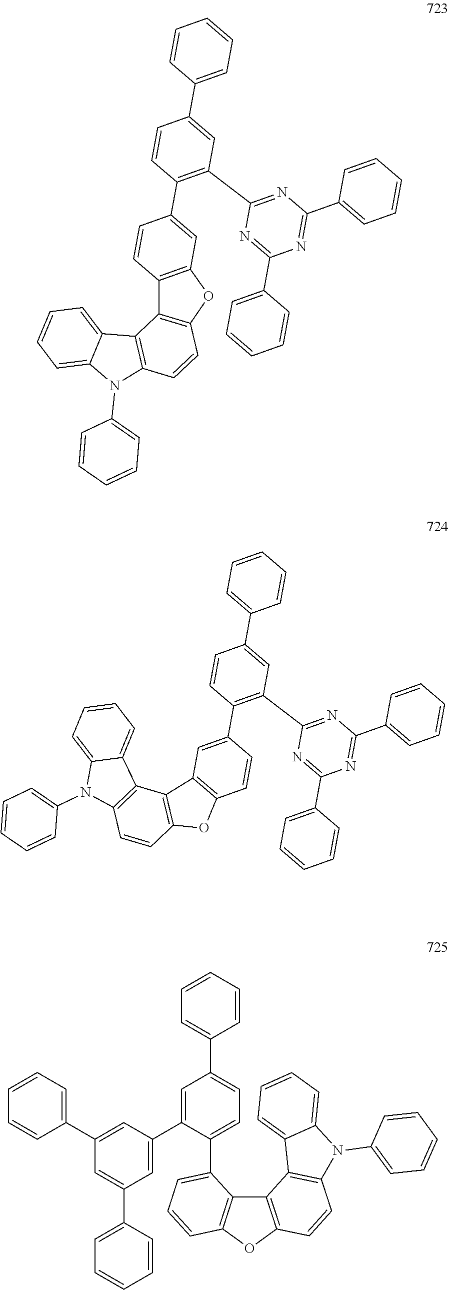 [SMILES: c1ccc(-c2ccc(-c3ccc4c(c3)oc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4oc5ccc6c(c7ccccc7n6-c6ccccc6)c5c4c3)c(-c3nc(-c4ccccc4)nc(-c4ccccc4)n3)c2)cc1 ; c1ccc(-c2cc(-c3ccccc3)cc(-c3cc(-c4ccccc4)ccc3-c3cccc4oc5ccc6c(c7ccccc7n6-c6ccccc6)c5c34)c2)cc1]

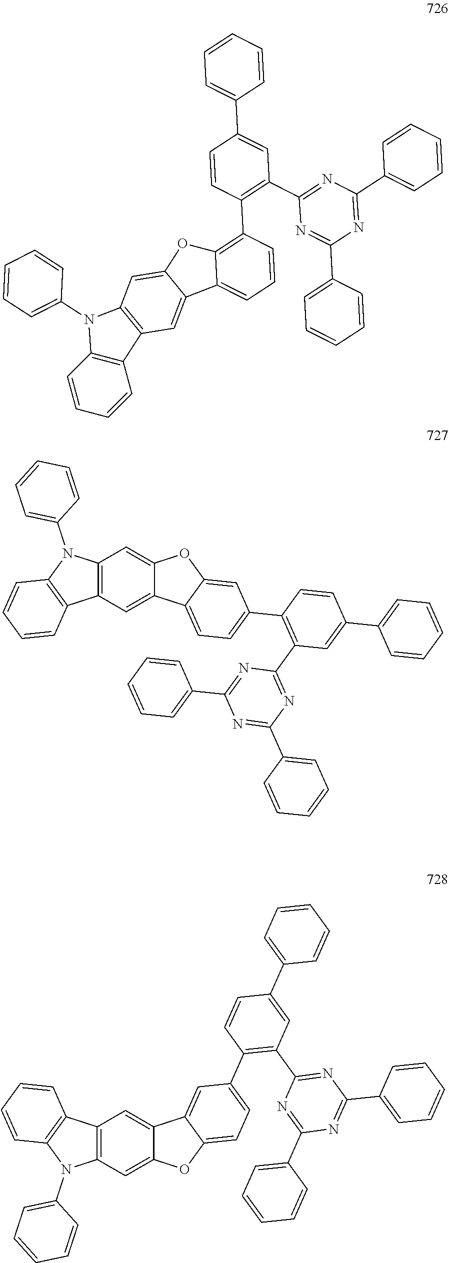 [SMILES: c1ccc(-c2ccc(-c3cccc4c3oc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)oc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4oc5cc6c(cc5c4c3)c3ccccc3n6-c3ccccc3)c(-c3nc(-c4ccccc4)nc(-c4ccccc4)n3)c2)cc1]

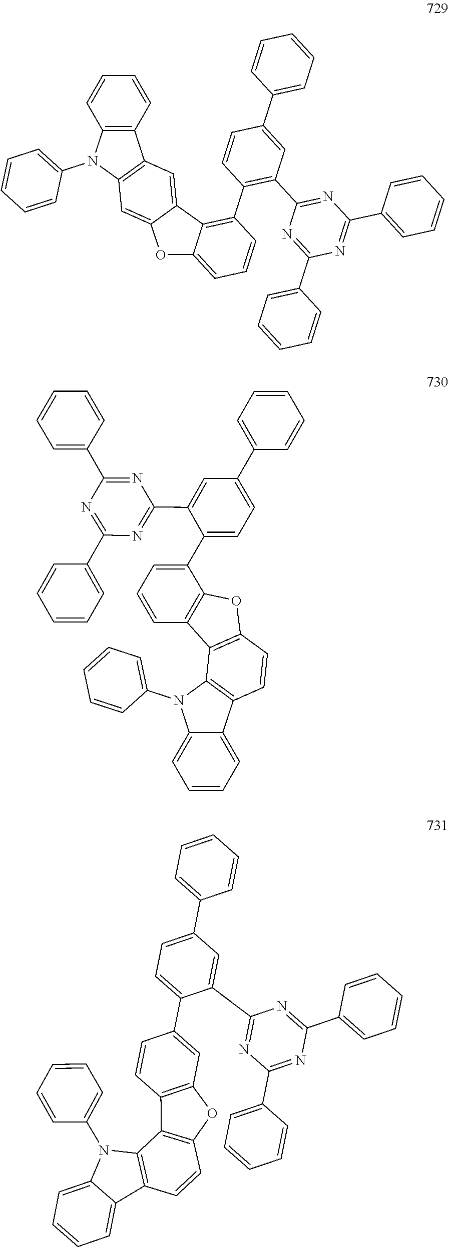

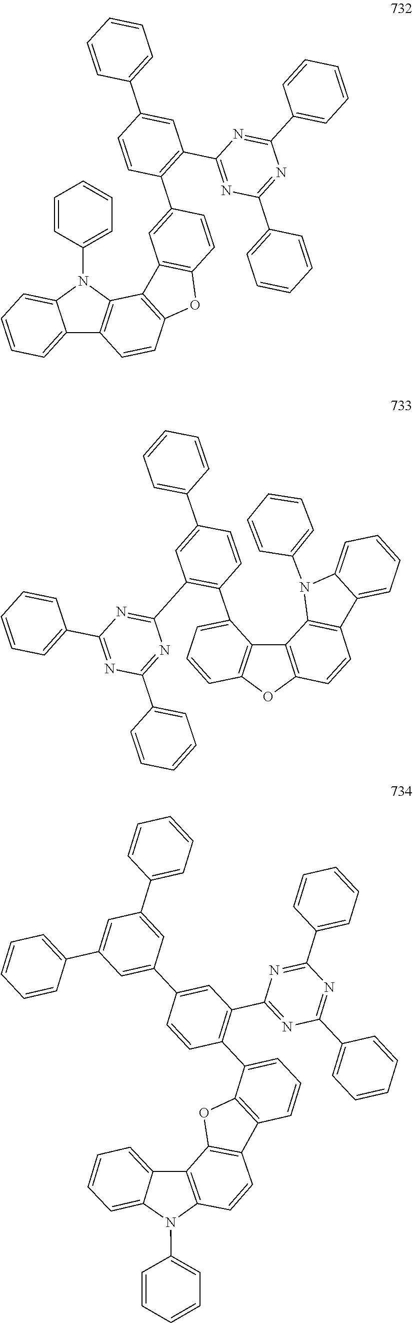

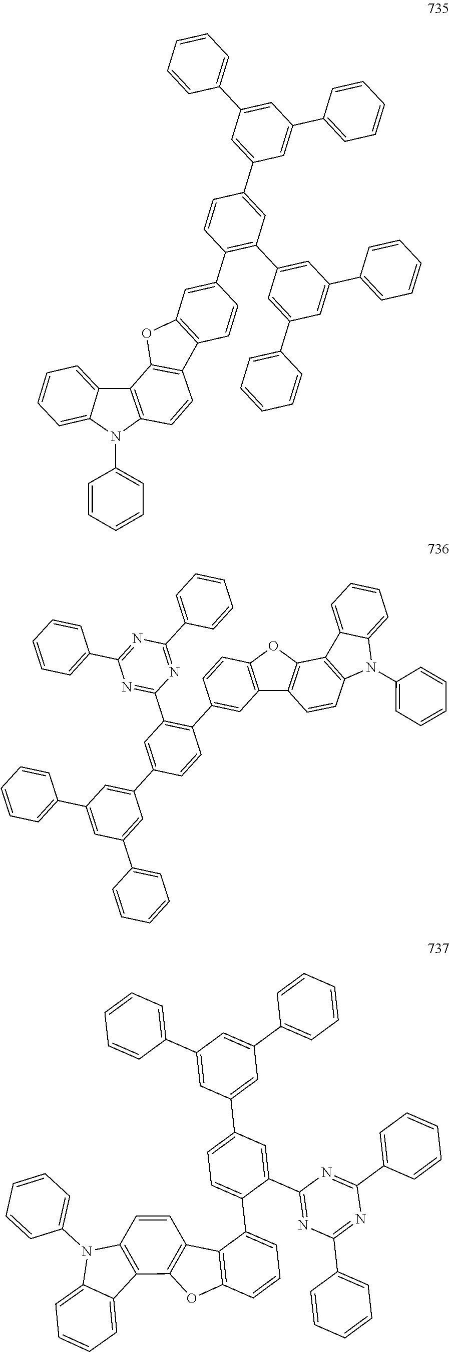 [SMILES: c1ccc(-c2cc(-c3ccccc3)cc(-c3ccc(-c4ccc5c(c4)oc4c5ccc5c4c4ccccc4n5-c4ccccc4)c(-c4cc(-c5ccccc5)cc(-c5ccccc5)c4)c3)c2)cc1 ; c1ccc(-c2cc(-c3ccccc3)cc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c6c6ccccc6n7-c6ccccc6)c45)c(-c4nc(-c5ccccc5)nc(-c5ccccc5)n4)c3)c2)cc1]

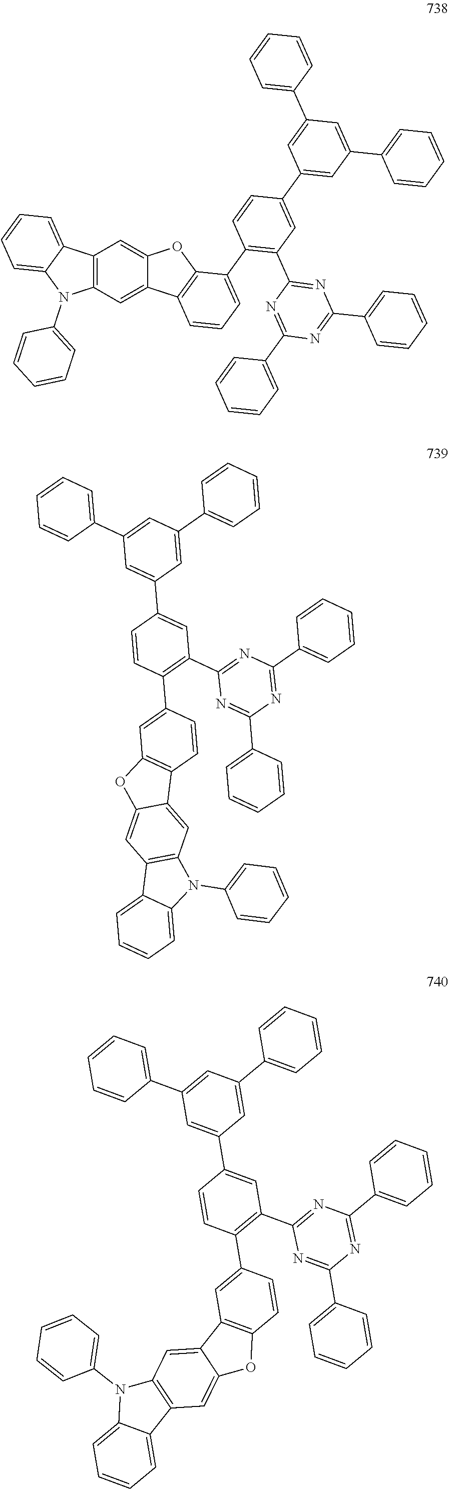 [SMILES: c1ccc(-c2cc(-c3ccccc3)cc(-c3ccc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1]

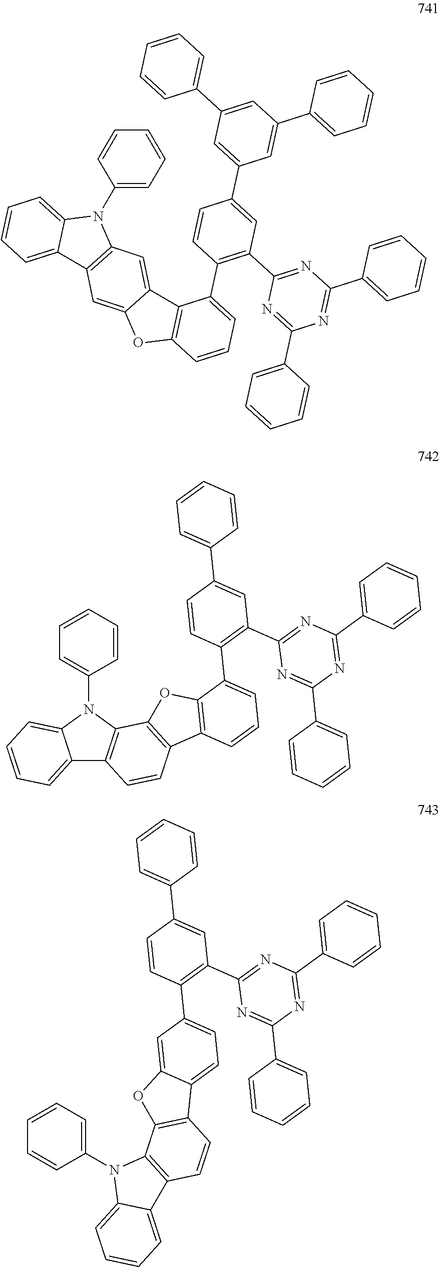

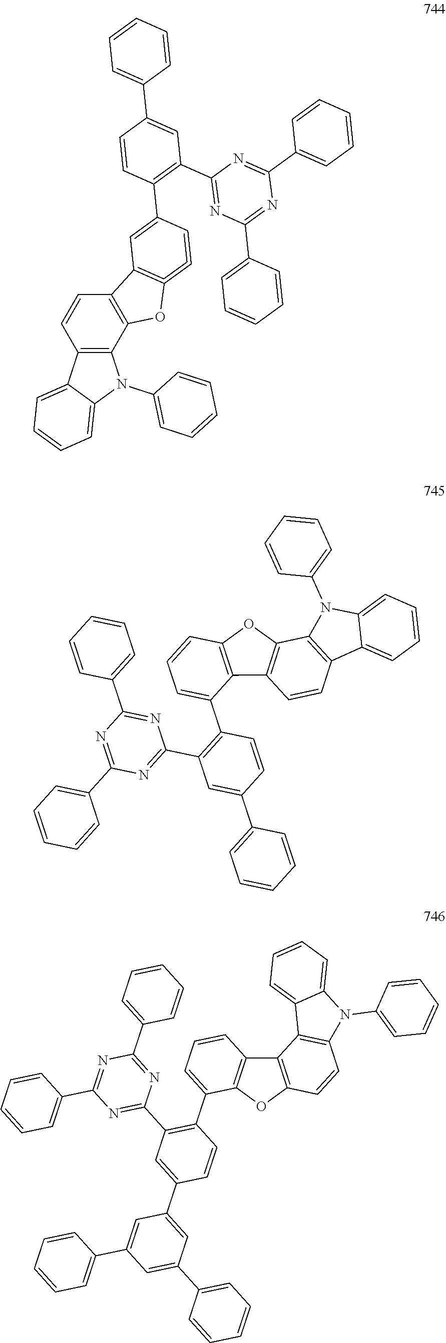

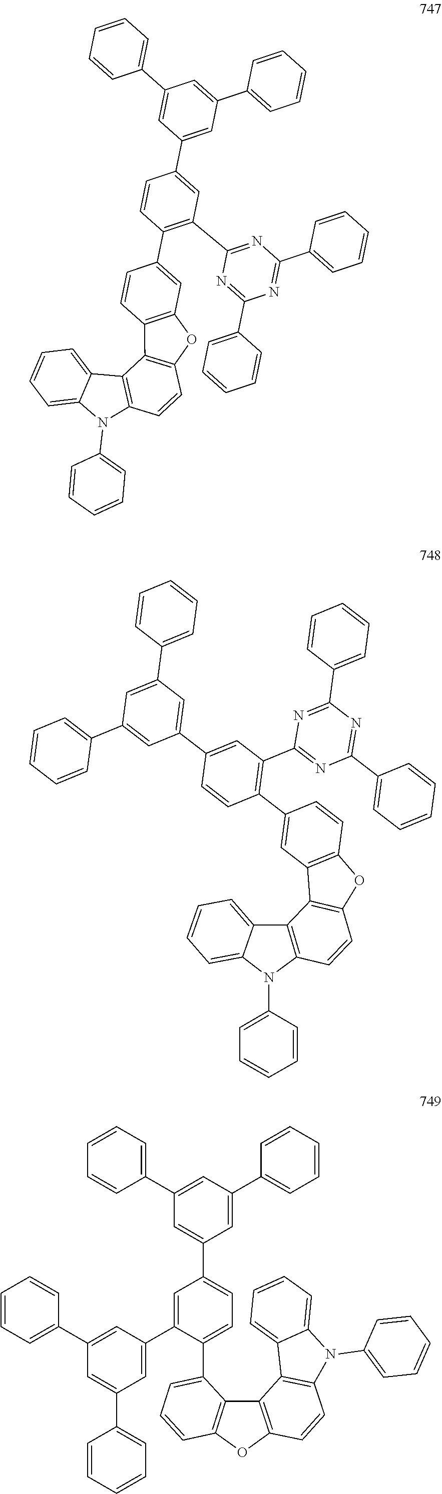 [SMILES: c1ccc(-c2cc(-c3ccccc3)cc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6ccc7c(c8ccccc8n7-c7ccccc7)c6c45)c(-c4cc(-c5ccccc5)cc(-c5ccccc5)c4)c3)c2)cc1]

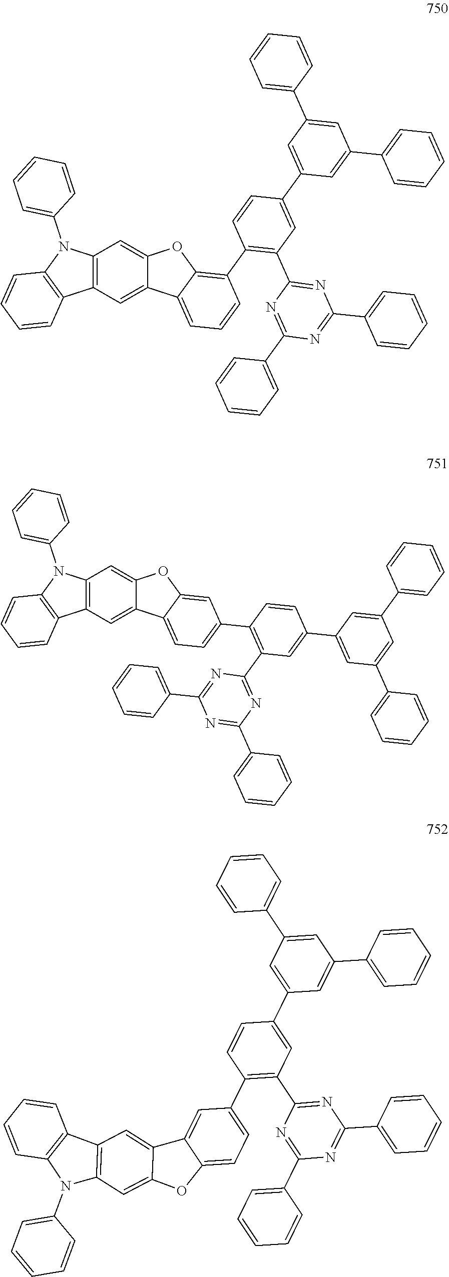 [SMILES: c1ccc(-c2cc(-c3ccccc3)cc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1]

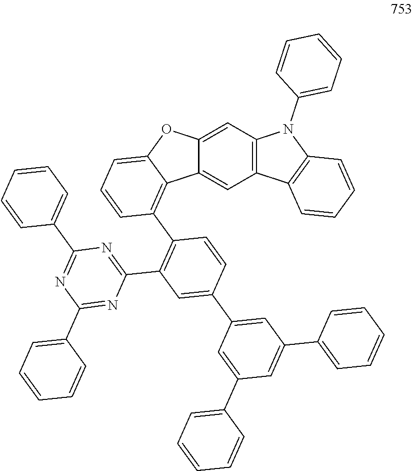 [SMILES: c1ccc(-c2cc(-c3ccccc3)cc(-c3ccc(-c4cccc5oc6cc7c(cc6c45)c4ccccc4n7-c4ccccc4)c(-c4nc(-c5ccccc5)nc(-c5ccccc5)n4)c3)c2)cc1]

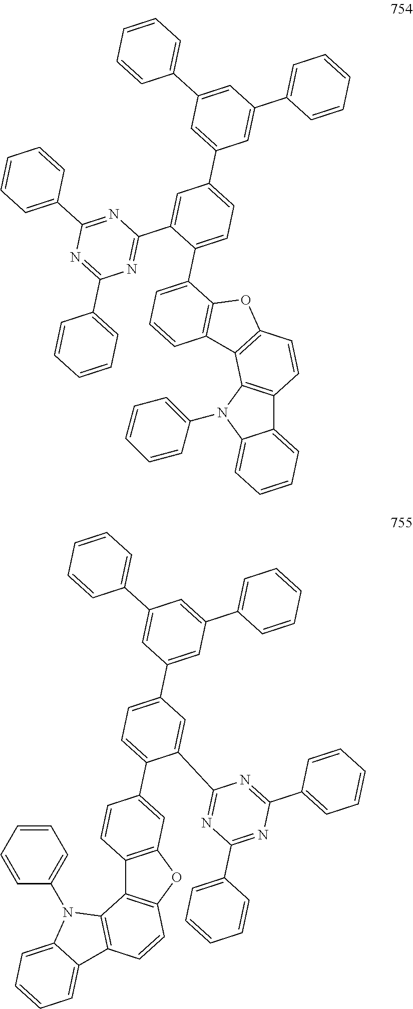 [SMILES: c1ccc(-c2cc(-c3ccccc3)cc(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1]

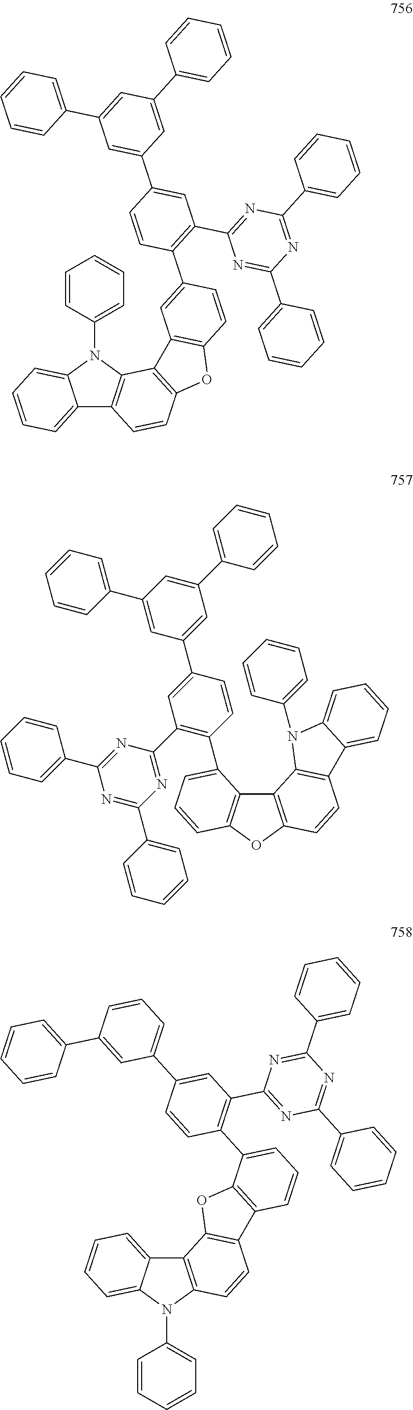 [SMILES: c1ccc(-c2cc(-c3ccccc3)cc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2cccc(-c3ccc(-c4cccc5c4oc4c5ccc5c4c4ccccc4n5-c4ccccc4)c(-c4nc(-c5ccccc5)nc(-c5ccccc5)n4)c3)c2)cc1]

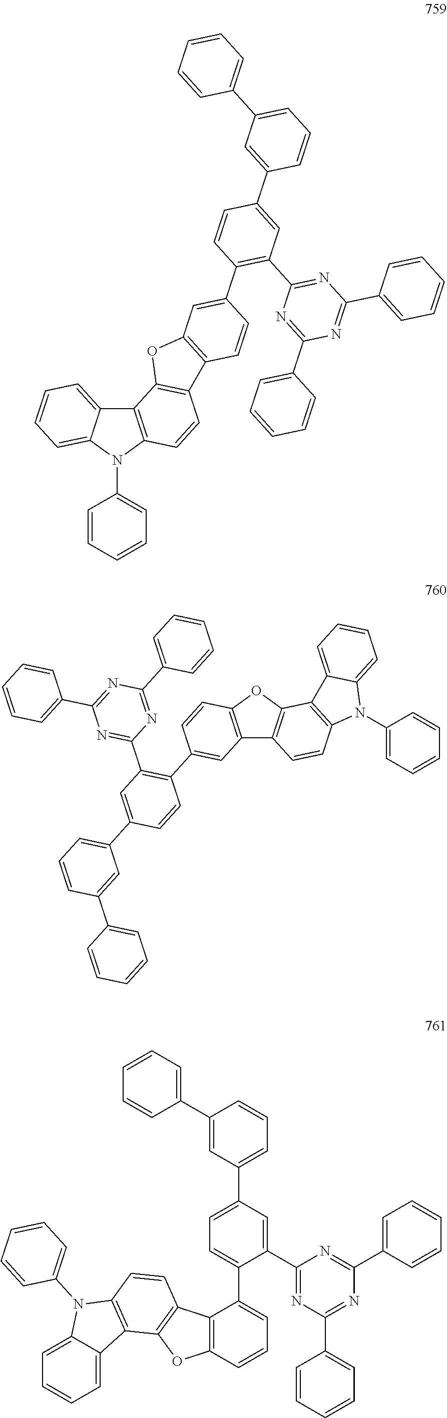 [SMILES: c1ccc(-c2cccc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2cccc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5oc6c(ccc7c6c6ccccc6n7-c6ccccc6)c45)c(-c4nc(-c5ccccc5)nc(-c5ccccc5)n4)c3)c2)cc1]

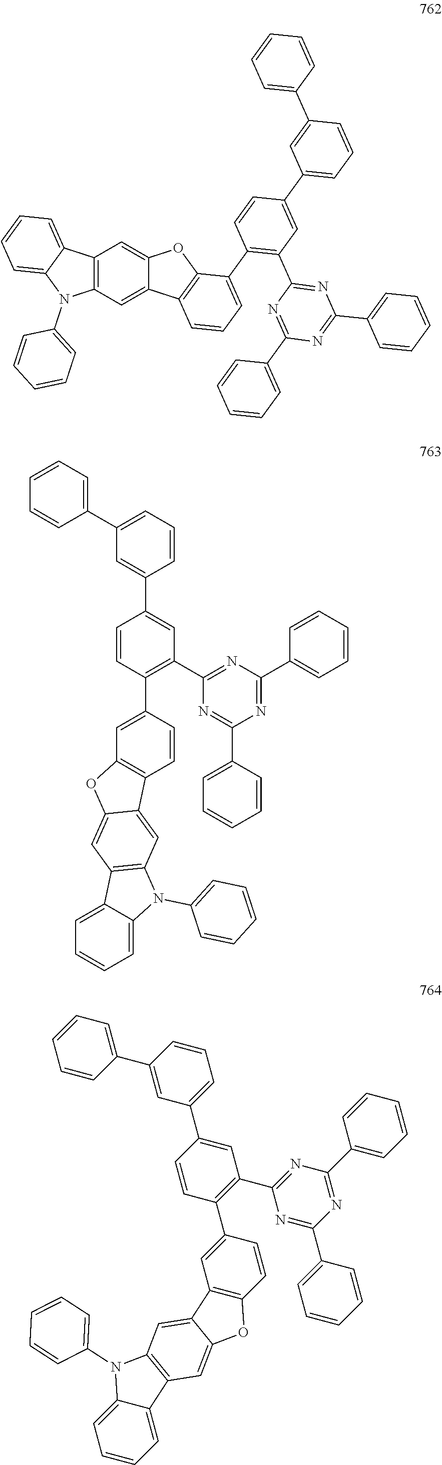 [SMILES: c1ccc(-c2cccc(-c3ccc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1]

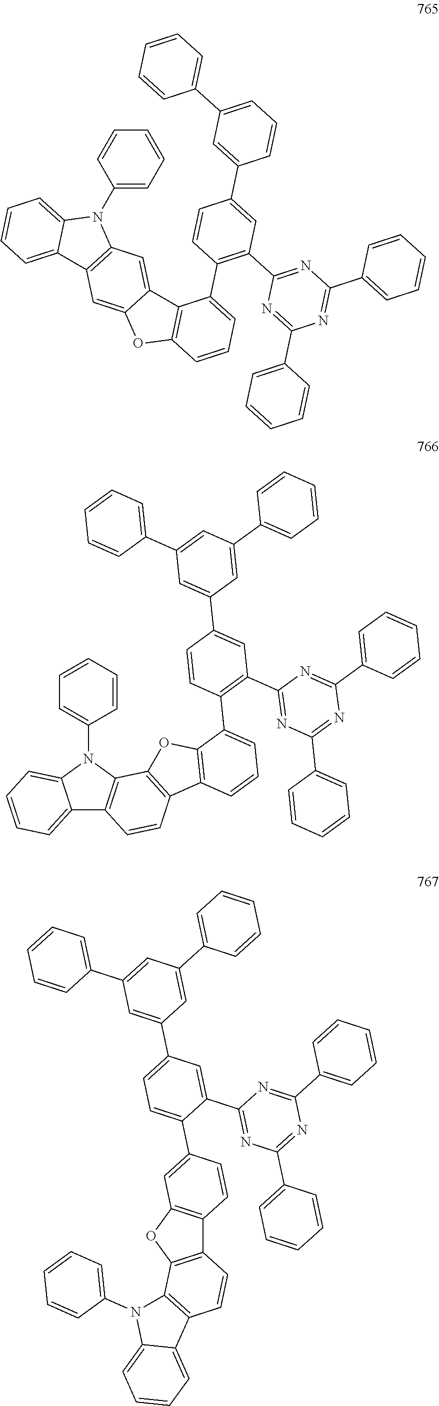

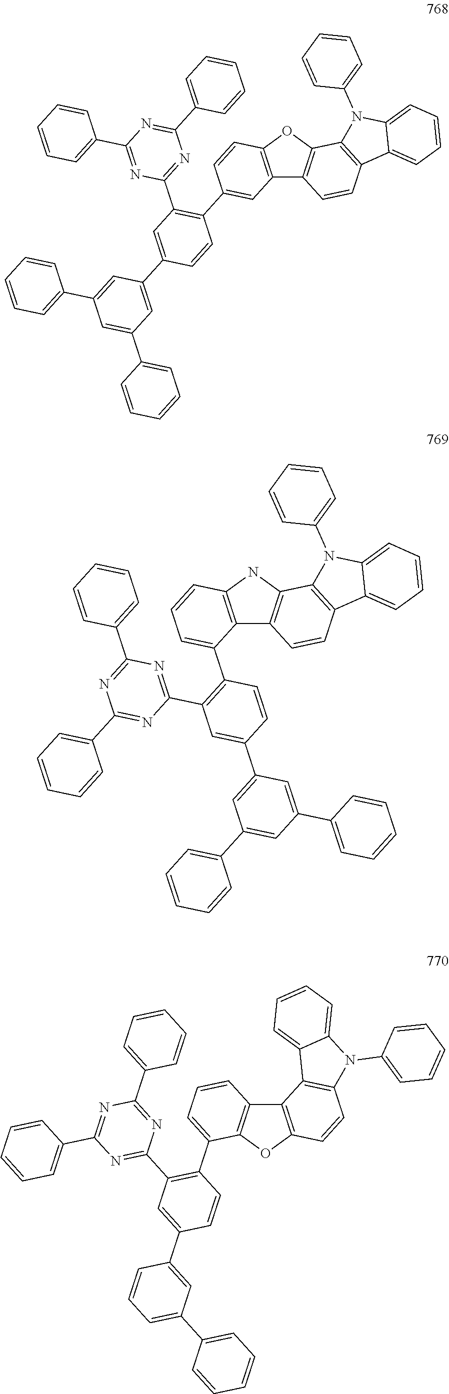 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3ccc(-c4cccc5[nH]c6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4oc4ccc6c(c7ccccc7n6-c6ccccc6)c45)c(-c4nc(-c5ccccc5)nc(-c5ccccc5)n4)c3)c2)cc1]

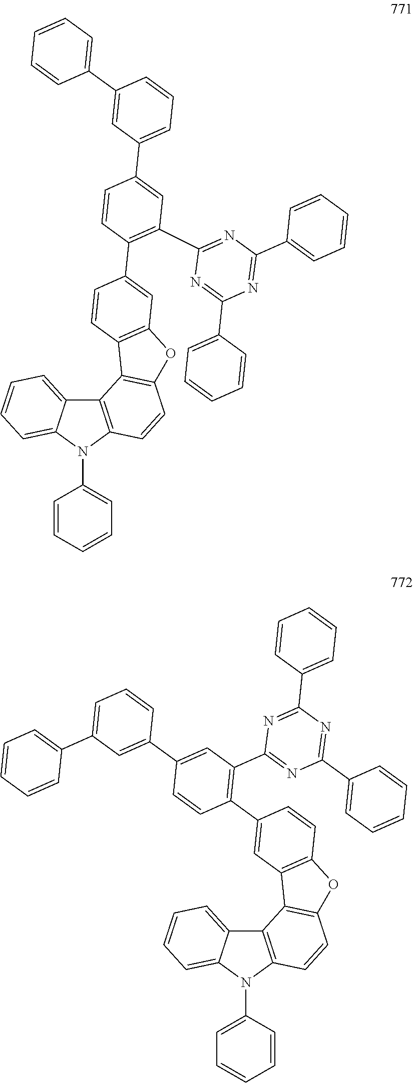 [SMILES: c1ccc(-c2cccc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1]

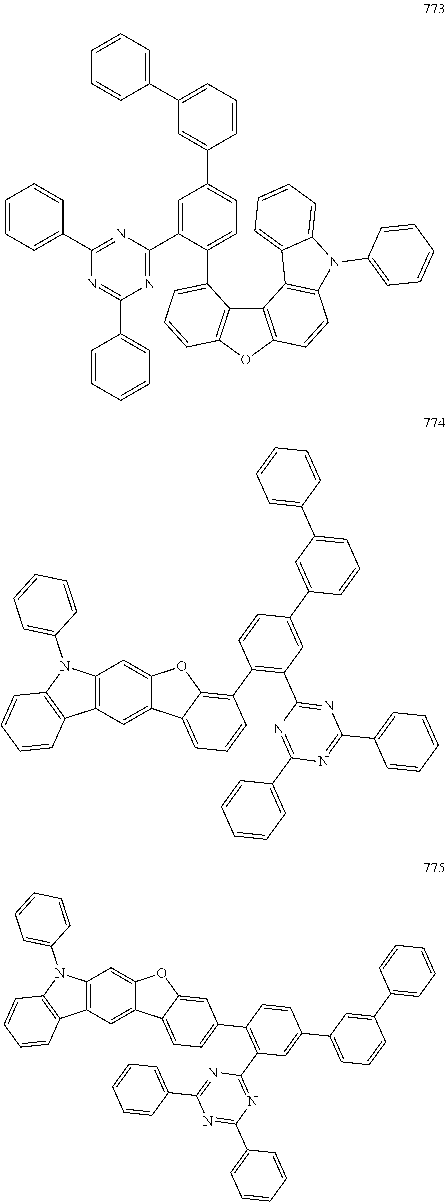 [SMILES: c1ccc(-c2cccc(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

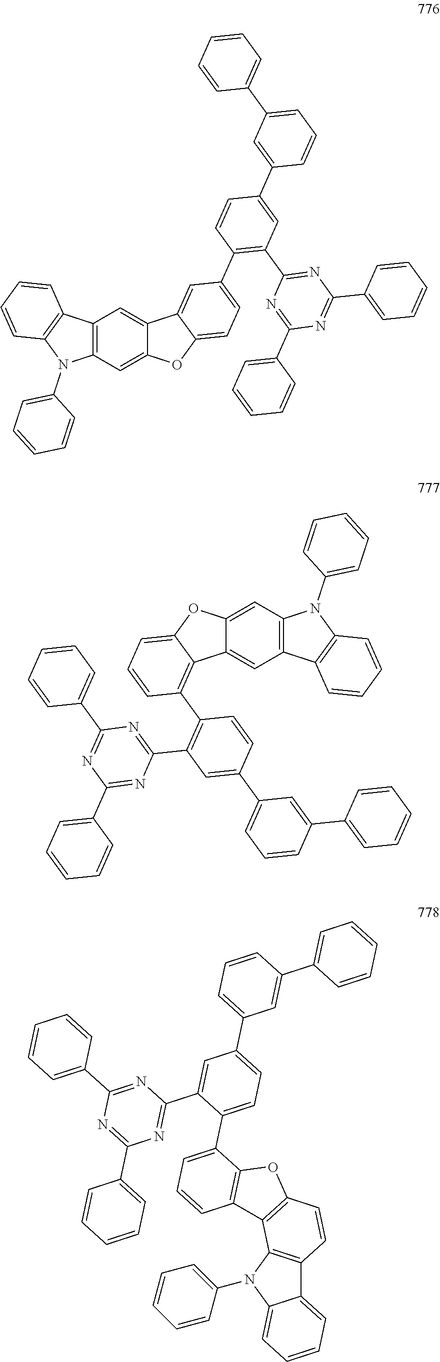 [SMILES: c1ccc(-c2cccc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5c4oc4ccc6c7ccccc7n(-c7ccccc7)c6c45)c(-c4nc(-c5ccccc5)nc(-c5ccccc5)n4)c3)c2)cc1]

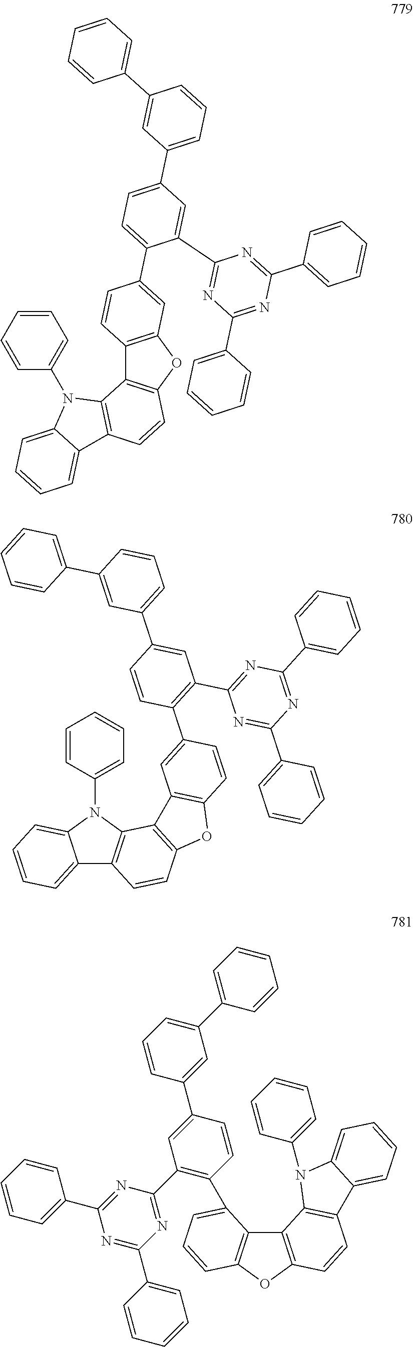

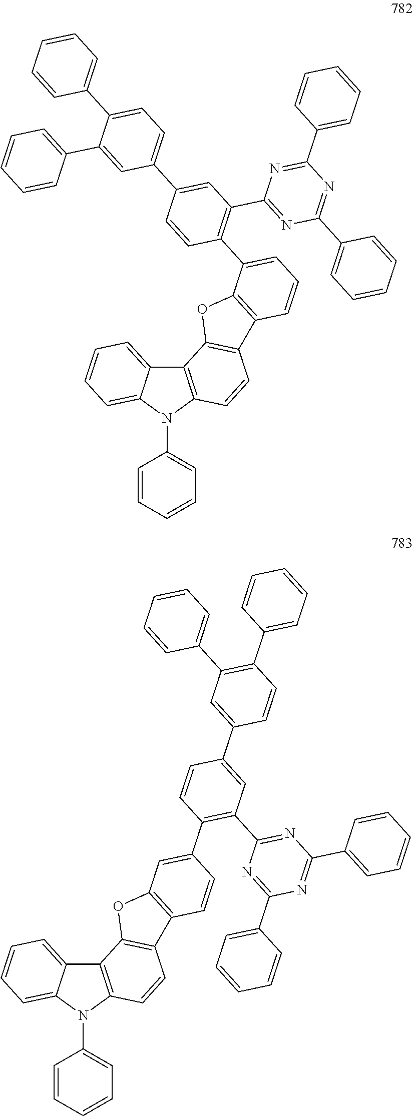 [SMILES: c1ccc(-c2nc(-c3ccccc3)nc(-c3cc(-c4ccc(-c5ccccc5)c(-c5ccccc5)c4)ccc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c3c3ccccc3n4-c3ccccc3)n2)cc1]

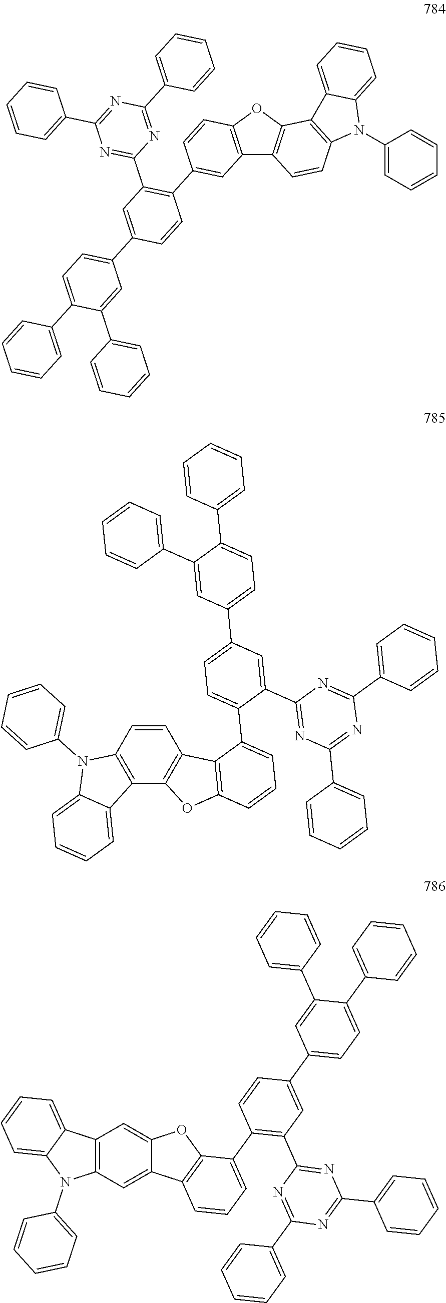 [SMILES: c1ccc(-c2nc(-c3ccccc3)nc(-c3cc(-c4ccc(-c5ccccc5)c(-c5ccccc5)c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c6ccccc6n(-c6ccccc6)c5cc34)n2)cc1]

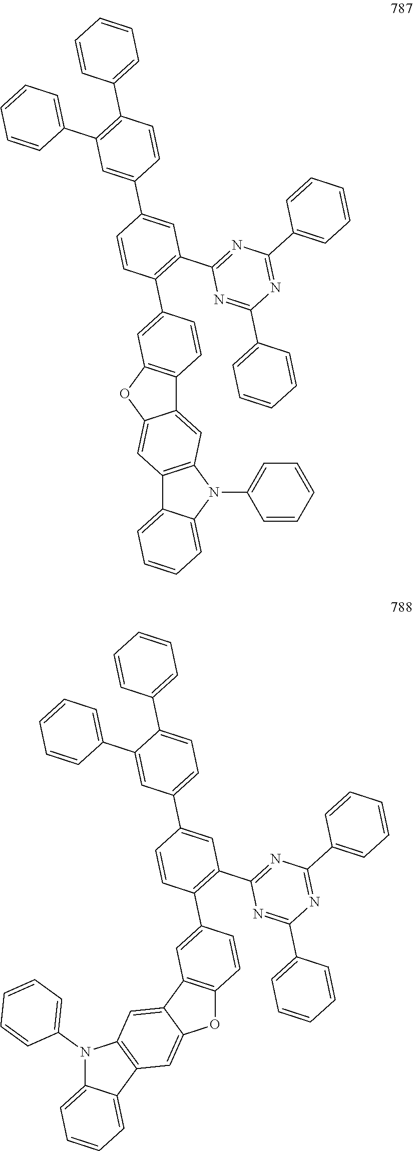 [SMILES: c1ccc(-c2nc(-c3ccccc3)nc(-c3cc(-c4ccc(-c5ccccc5)c(-c5ccccc5)c4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)ccc3-c3ccc4oc5cc6c7ccccc7n(-c7ccccc7)c6cc5c4c3)n2)cc1]

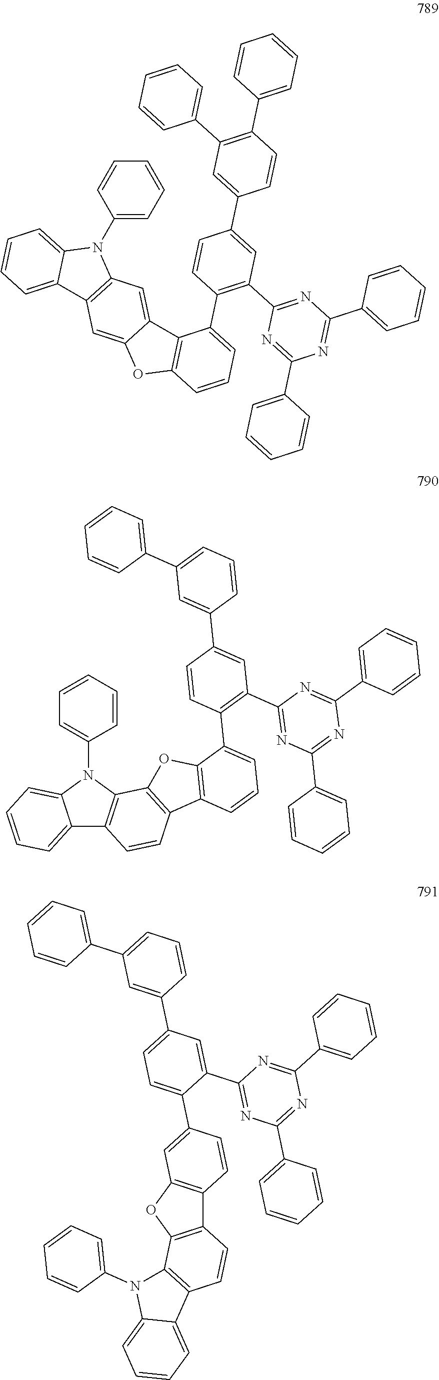 [SMILES: c1ccc(-c2nc(-c3ccccc3)nc(-c3cc(-c4ccc(-c5ccccc5)c(-c5ccccc5)c4)ccc3-c3cccc4oc5cc6c7ccccc7n(-c7ccccc7)c6cc5c34)n2)cc1 ; c1ccc(-c2cccc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1]

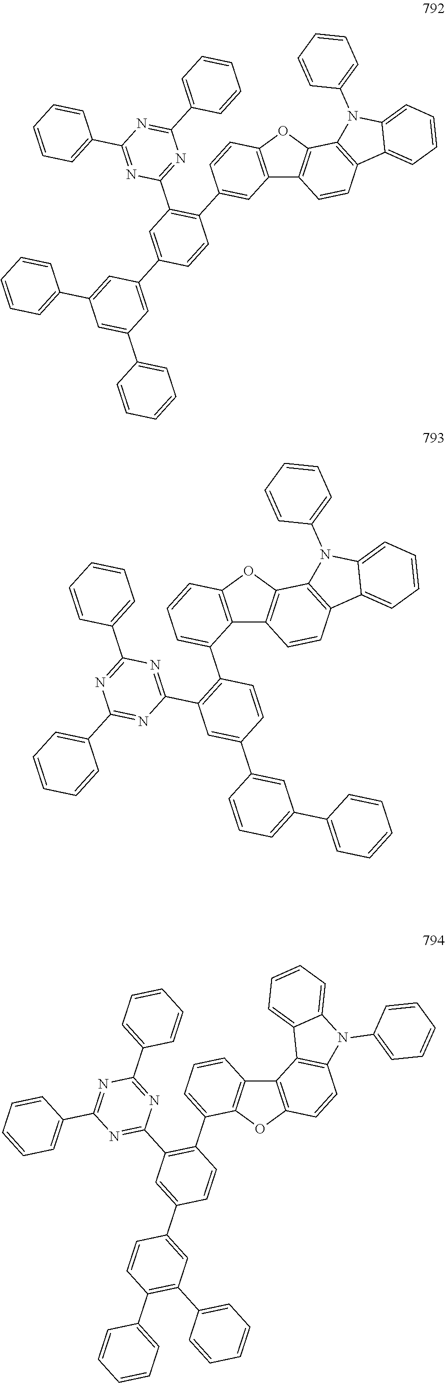 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3oc3ccc5c(c6ccccc6n5-c5ccccc5)c34)n2)cc1]

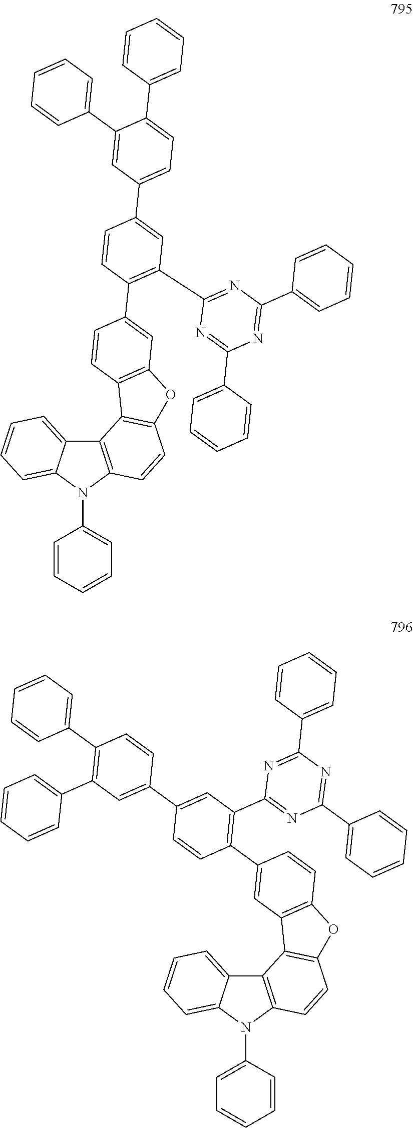 [SMILES: c1ccc(-c2nc(-c3ccccc3)nc(-c3cc(-c4ccc(-c5ccccc5)c(-c5ccccc5)c4)ccc3-c3ccc4c(c3)oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4oc5ccc6c(c7ccccc7n6-c6ccccc6)c5c4c3)n2)cc1]

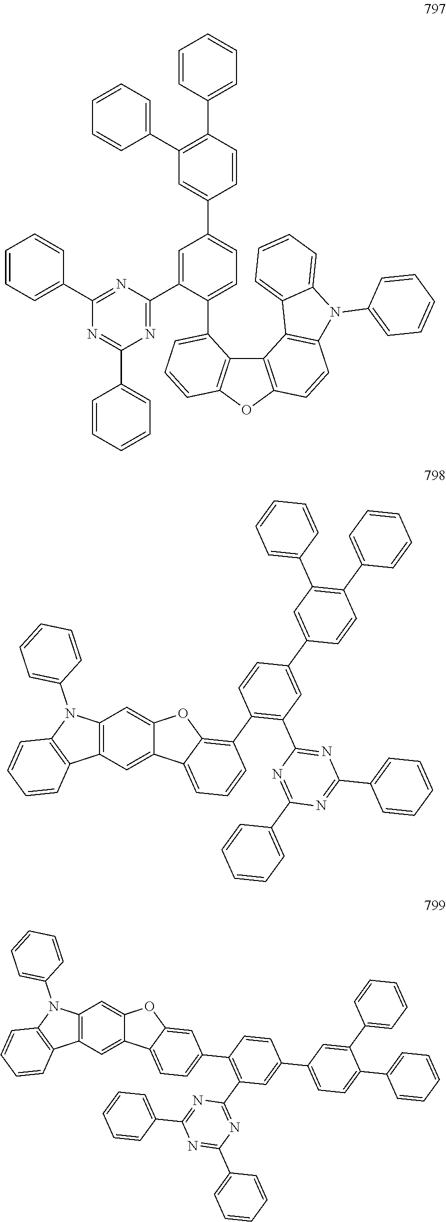 [SMILES: c1ccc(-c2nc(-c3ccccc3)nc(-c3cc(-c4ccc(-c5ccccc5)c(-c5ccccc5)c4)ccc3-c3cccc4oc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3cc5c(cc34)c3ccccc3n5-c3ccccc3)n2)cc1]

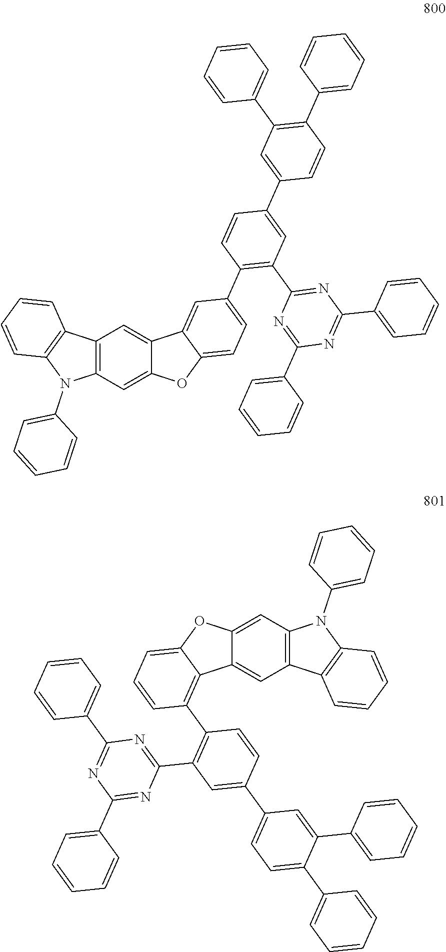 [SMILES: c1ccc(-c2nc(-c3ccccc3)nc(-c3cc(-c4ccc(-c5ccccc5)c(-c5ccccc5)c4)ccc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4oc5cc6c(cc5c34)c3ccccc3n6-c3ccccc3)n2)cc1]

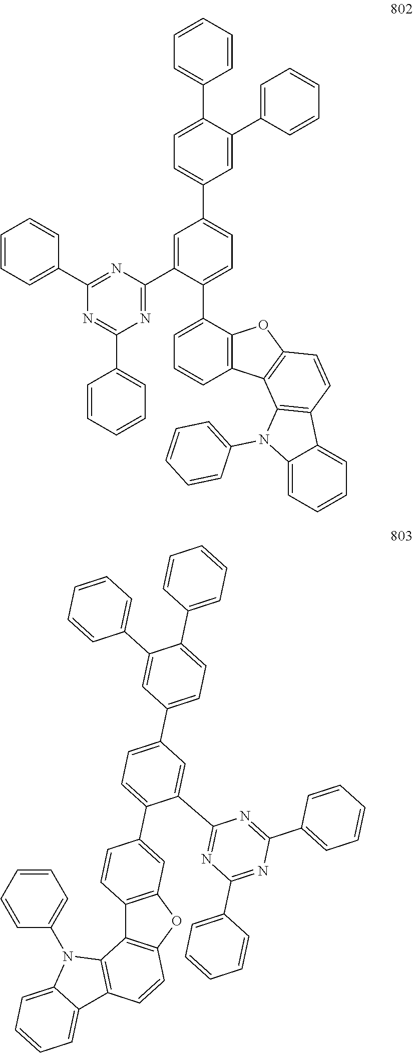 [SMILES: c1ccc(-c2nc(-c3ccccc3)nc(-c3cc(-c4ccc(-c5ccccc5)c(-c5ccccc5)c4)ccc3-c3cccc4c3oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3ccc5c6ccccc6n(-c6ccccc6)c5c34)n2)cc1]

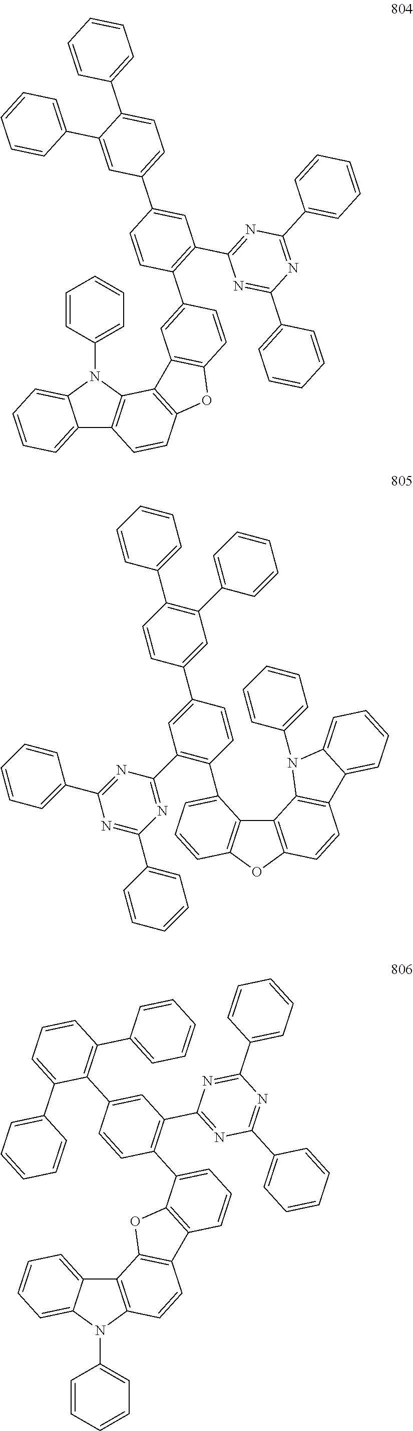 [SMILES: c1ccc(-c2nc(-c3ccccc3)nc(-c3cc(-c4ccc(-c5ccccc5)c(-c5ccccc5)c4)ccc3-c3ccc4oc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)ccc3-c3cccc4oc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)ccc3-c3cccc4c3oc3c4ccc4c3c3ccccc3n4-c3ccccc3)n2)cc1]

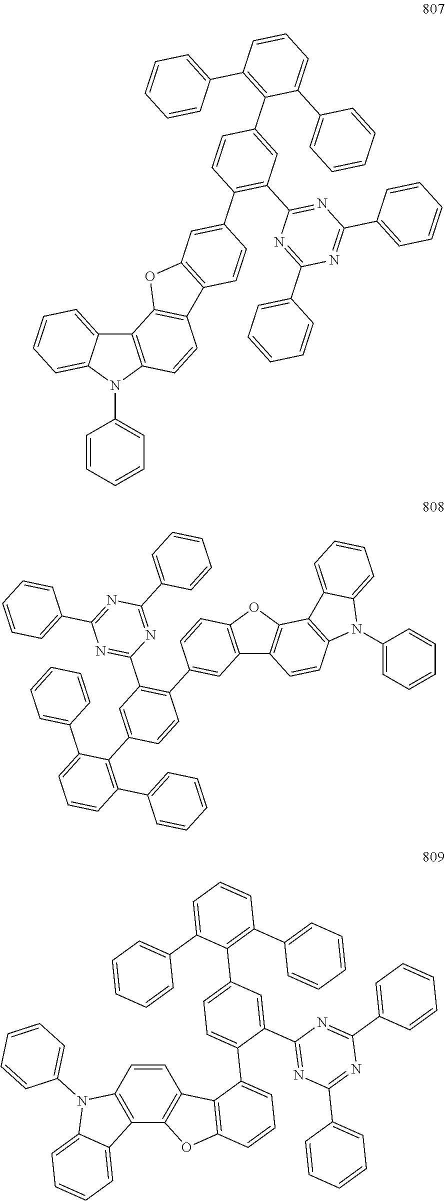 [SMILES: c1ccc(-c2nc(-c3ccccc3)nc(-c3cc(-c4c(-c5ccccc5)cccc4-c4ccccc4)ccc3-c3ccc4c(c3)oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)ccc3-c3cccc4oc5c(ccc6c5c5ccccc5n6-c5ccccc5)c34)n2)cc1]

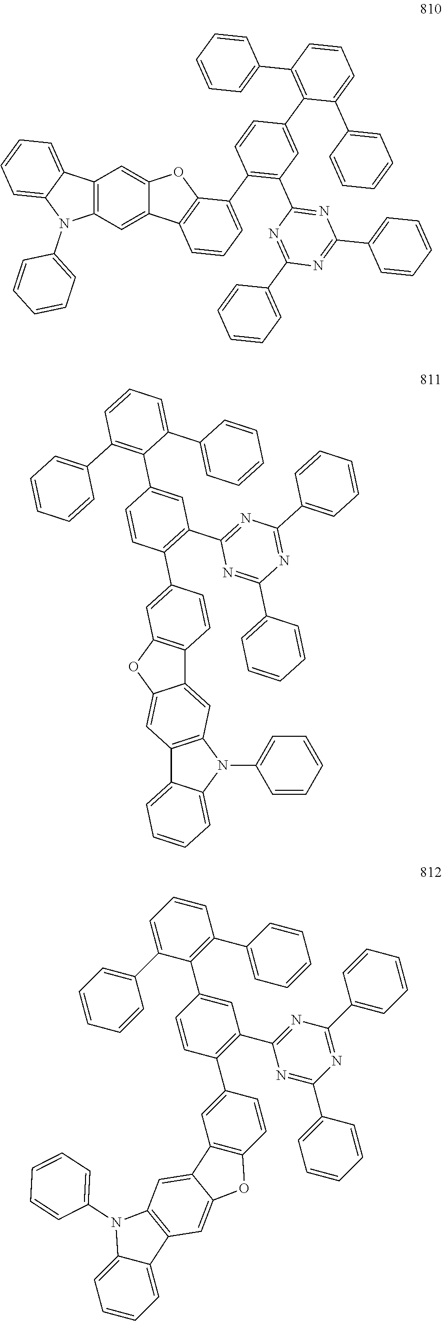 [SMILES: c1ccc(-c2nc(-c3ccccc3)nc(-c3cc(-c4c(-c5ccccc5)cccc4-c4ccccc4)ccc3-c3cccc4c3oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4oc5cc6c7ccccc7n(-c7ccccc7)c6cc5c4c3)n2)cc1]

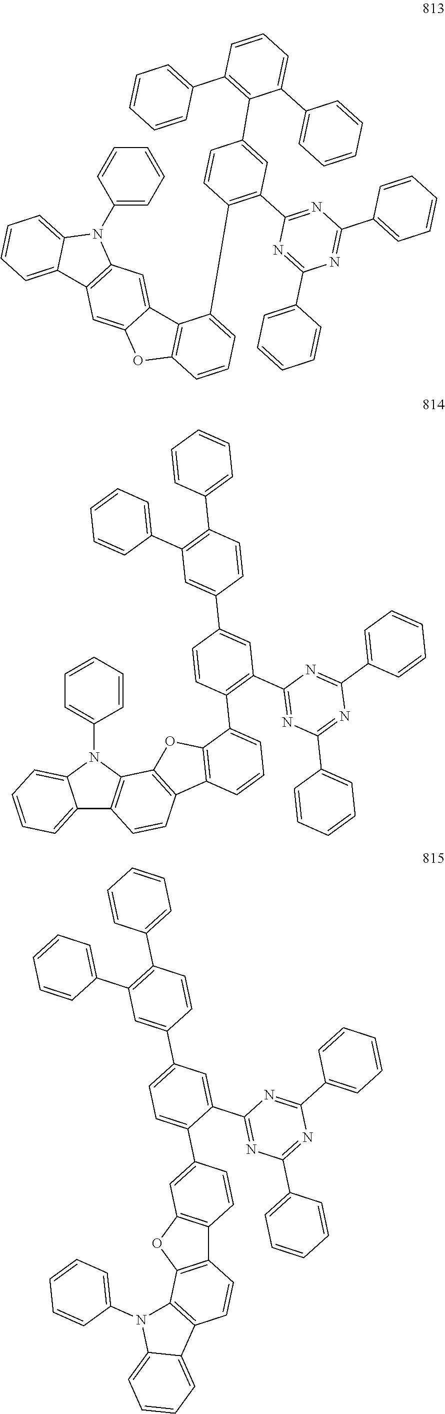

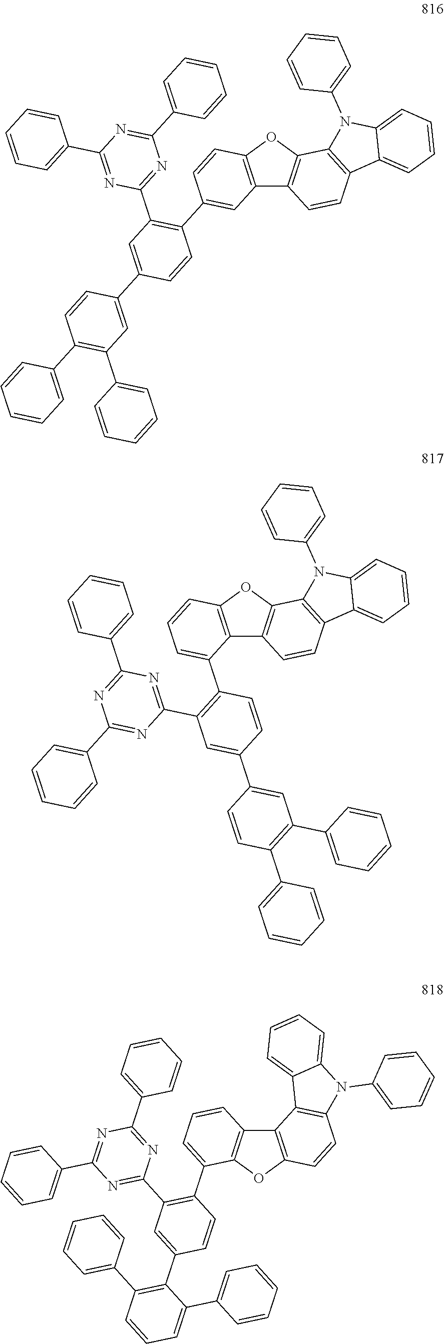 [SMILES: c1ccc(-c2nc(-c3ccccc3)nc(-c3cc(-c4ccc(-c5ccccc5)c(-c5ccccc5)c4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)ccc3-c3cccc4c3oc3ccc5c(c6ccccc6n5-c5ccccc5)c34)n2)cc1]

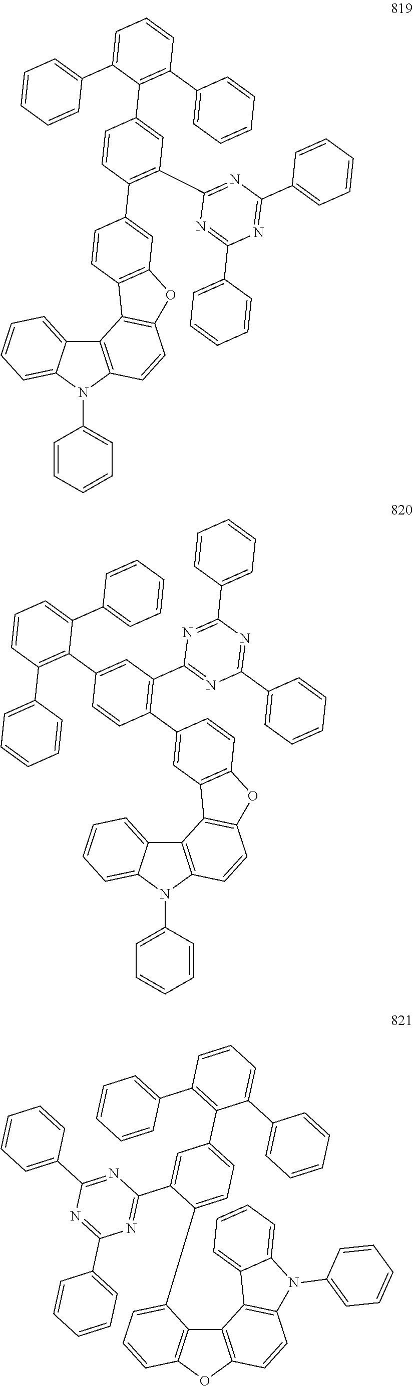 [SMILES: c1ccc(-c2nc(-c3ccccc3)nc(-c3cc(-c4c(-c5ccccc5)cccc4-c4ccccc4)ccc3-c3ccc4c(c3)oc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)ccc3-c3ccc4oc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)ccc3-c3cccc4oc5ccc6c(c7ccccc7n6-c6ccccc6)c5c34)n2)cc1]

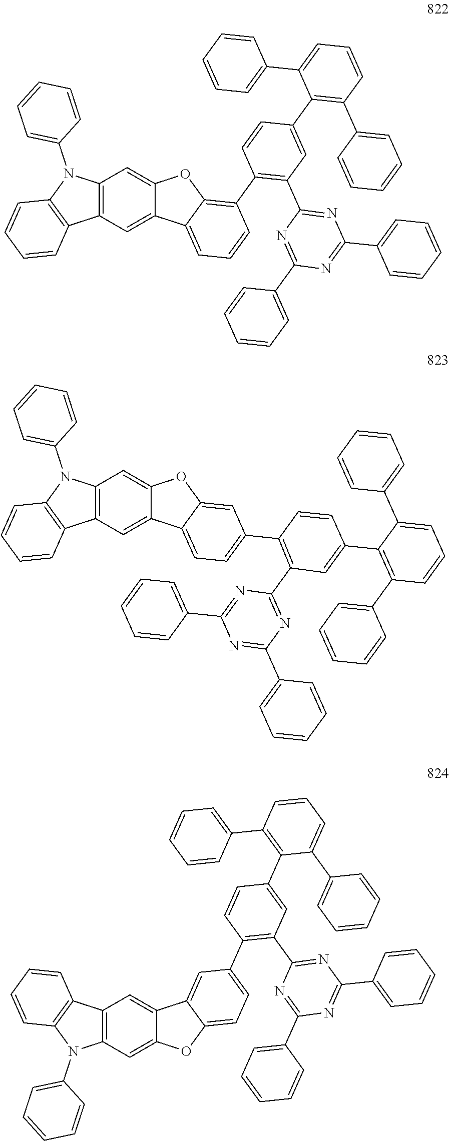 [SMILES: c1ccc(-c2nc(-c3ccccc3)nc(-c3cc(-c4c(-c5ccccc5)cccc4-c4ccccc4)ccc3-c3cccc4c3oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4oc5cc6c(cc5c4c3)c3ccccc3n6-c3ccccc3)n2)cc1]

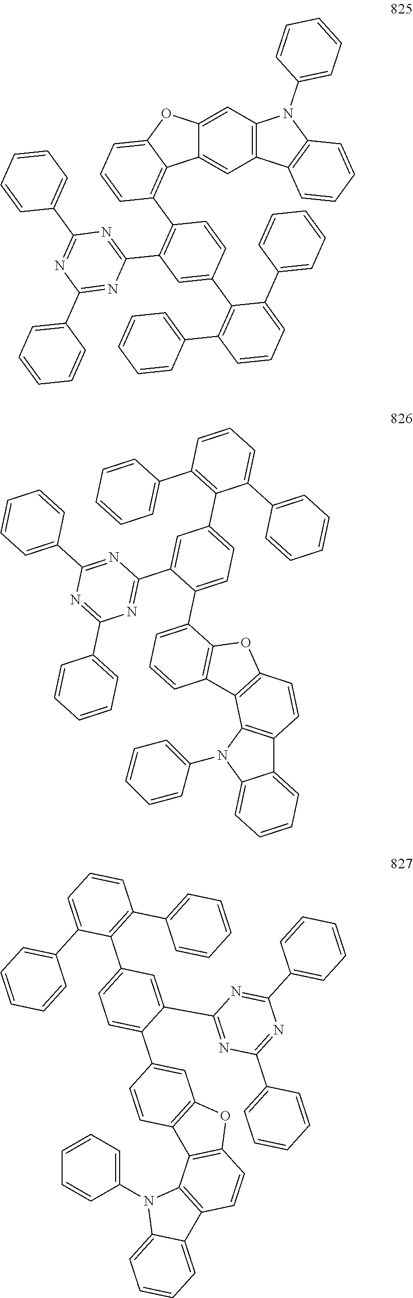 [SMILES: c1ccc(-c2nc(-c3ccccc3)nc(-c3cc(-c4c(-c5ccccc5)cccc4-c4ccccc4)ccc3-c3cccc4oc5cc6c(cc5c34)c3ccccc3n6-c3ccccc3)n2)cc1 ; c1ccc(-c2nc(-c3ccccc3)nc(-c3cc(-c4c(-c5ccccc5)cccc4-c4ccccc4)ccc3-c3cccc4c3oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)ccc3-c3ccc4c(c3)oc3ccc5c6ccccc6n(-c6ccccc6)c5c34)n2)cc1]

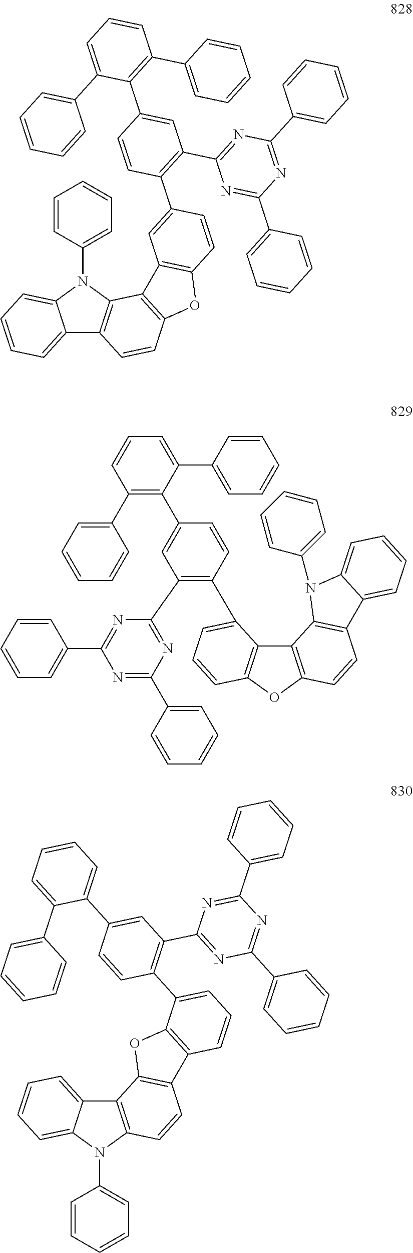 [SMILES: c1ccc(-c2nc(-c3ccccc3)nc(-c3cc(-c4c(-c5ccccc5)cccc4-c4ccccc4)ccc3-c3ccc4oc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)ccc3-c3cccc4oc5ccc6c7ccccc7n(-c7ccccc7)c6c5c34)n2)cc1 ; c1ccc(-c2nc(-c3ccccc3)nc(-c3cc(-c4ccccc4-c4ccccc4)ccc3-c3cccc4c3oc3c4ccc4c3c3ccccc3n4-c3ccccc3)n2)cc1]

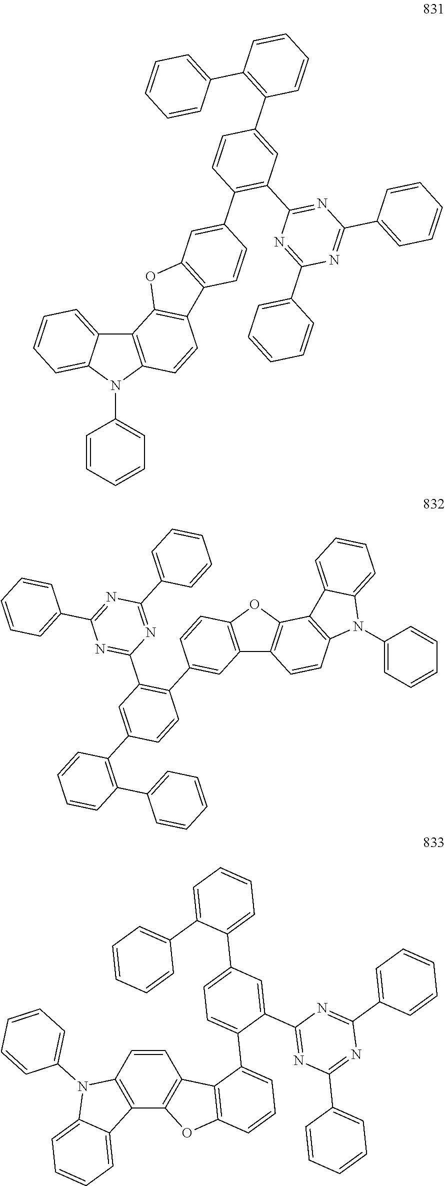 [SMILES: c1ccc(-c2nc(-c3ccccc3)nc(-c3cc(-c4ccccc4-c4ccccc4)ccc3-c3ccc4c(c3)oc3c4ccc4c3c3ccccc3n4-c3ccccc3)n2)cc1 ; c1ccc(-c2nc(-c3ccccc3)nc(-c3cc(-c4ccccc4-c4ccccc4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccccc4-c4ccccc4)ccc3-c3cccc4oc5c(ccc6c5c5ccccc5n6-c5ccccc5)c34)n2)cc1]

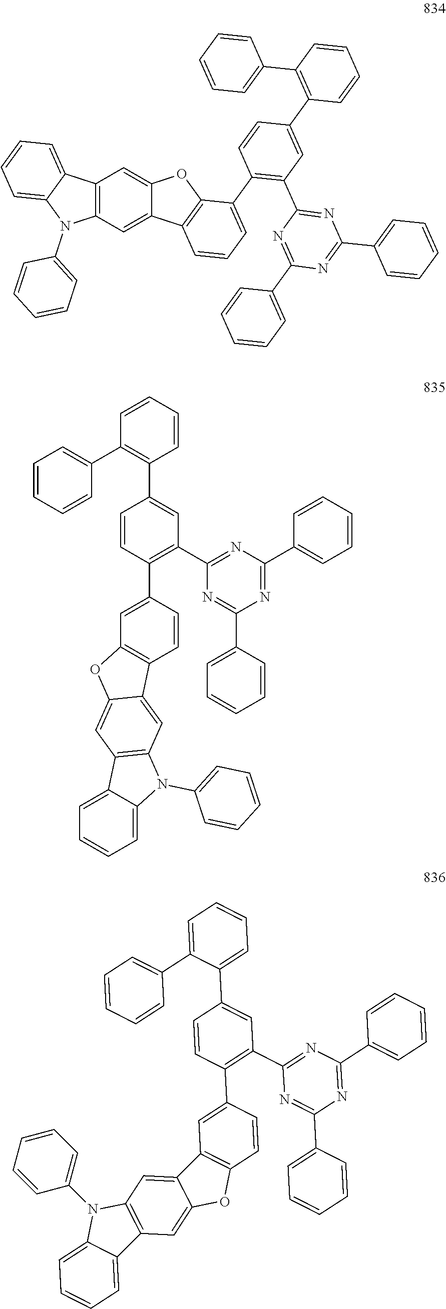 [SMILES: c1ccc(-c2nc(-c3ccccc3)nc(-c3cc(-c4ccccc4-c4ccccc4)ccc3-c3cccc4c3oc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4oc5cc6c7ccccc7n(-c7ccccc7)c6cc5c4c3)n2)cc1]

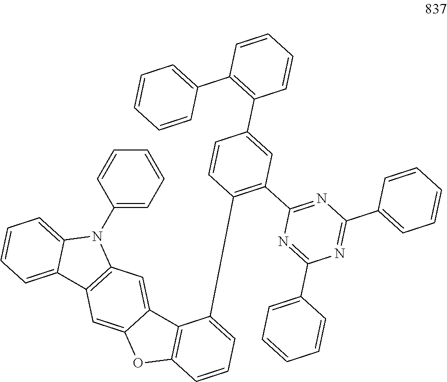 [SMILES: c1ccc(-c2nc(-c3ccccc3)nc(-c3cc(-c4ccccc4-c4ccccc4)ccc3-c3cccc4oc5cc6c7ccccc7n(-c7ccccc7)c6cc5c34)n2)cc1]

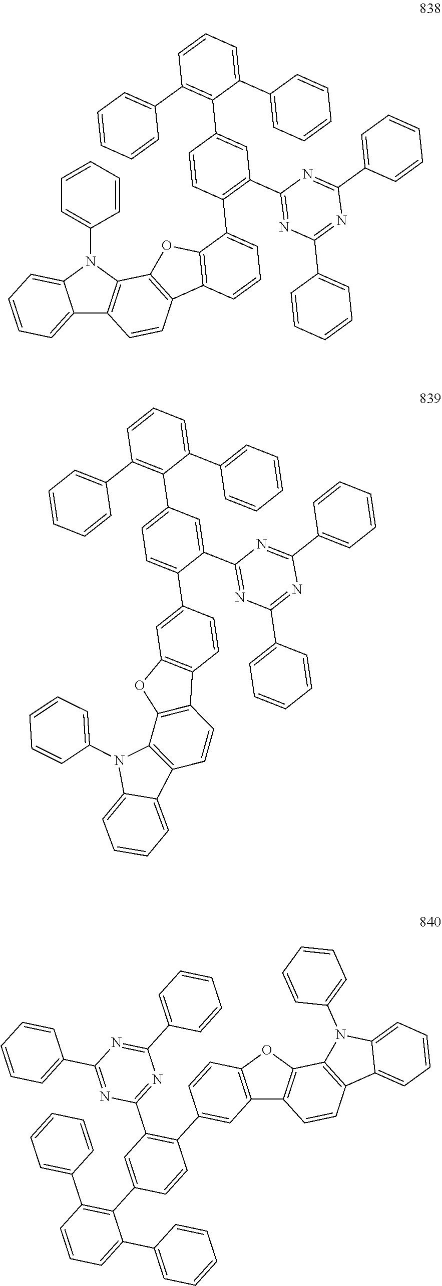 [SMILES: c1ccc(-c2nc(-c3ccccc3)nc(-c3cc(-c4c(-c5ccccc5)cccc4-c4ccccc4)ccc3-c3cccc4c3oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4c(c3)oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4oc5c(ccc6c7ccccc7n(-c7ccccc7)c65)c4c3)n2)cc1]

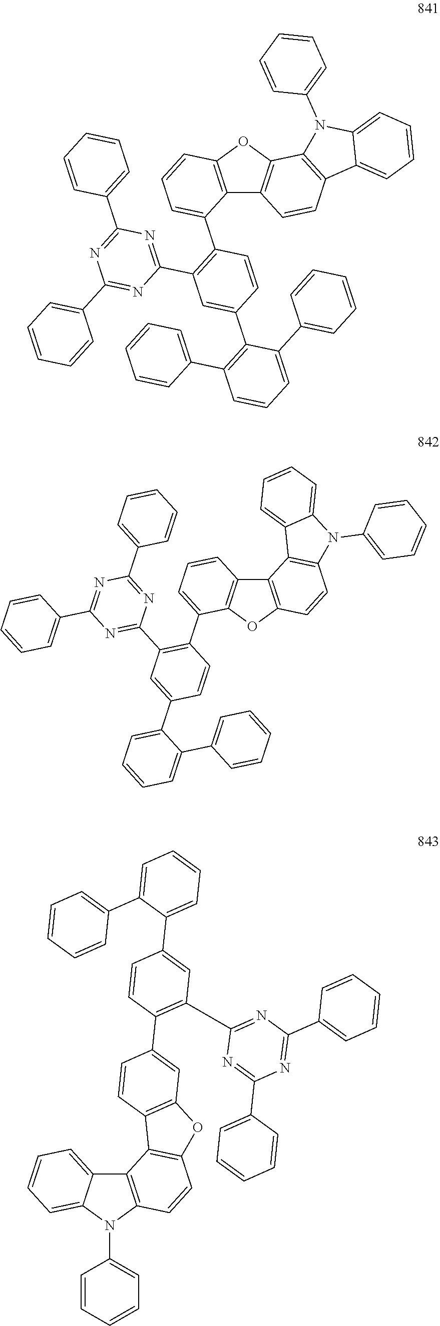 [SMILES: c1ccc(-c2nc(-c3ccccc3)nc(-c3cc(-c4c(-c5ccccc5)cccc4-c4ccccc4)ccc3-c3cccc4oc5c(ccc6c7ccccc7n(-c7ccccc7)c65)c34)n2)cc1 ; c1ccc(-c2nc(-c3ccccc3)nc(-c3cc(-c4ccccc4-c4ccccc4)ccc3-c3cccc4c3oc3ccc5c(c6ccccc6n5-c5ccccc5)c34)n2)cc1 ; c1ccc(-c2nc(-c3ccccc3)nc(-c3cc(-c4ccccc4-c4ccccc4)ccc3-c3ccc4c(c3)oc3ccc5c(c6ccccc6n5-c5ccccc5)c34)n2)cc1]

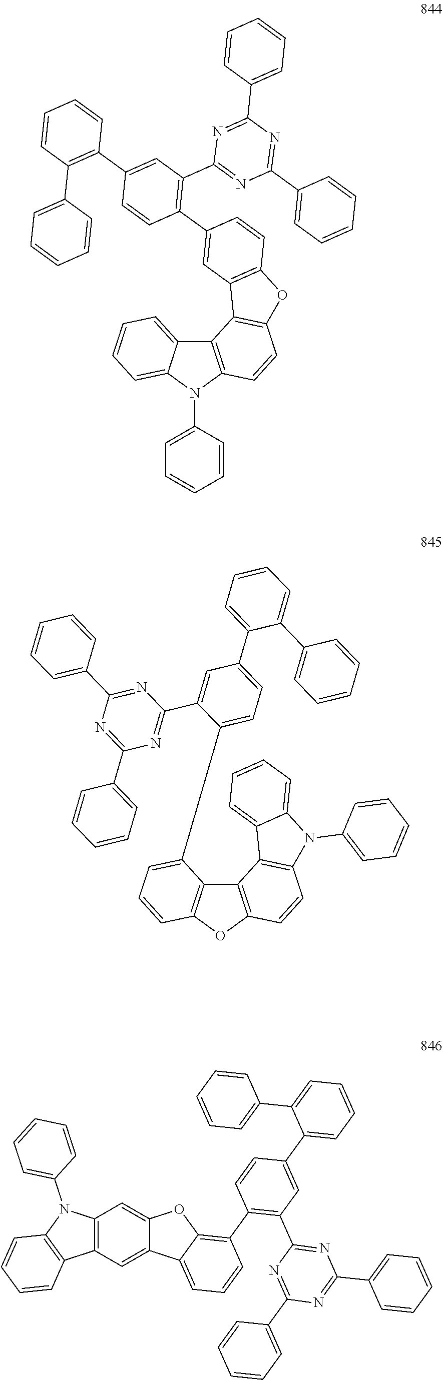

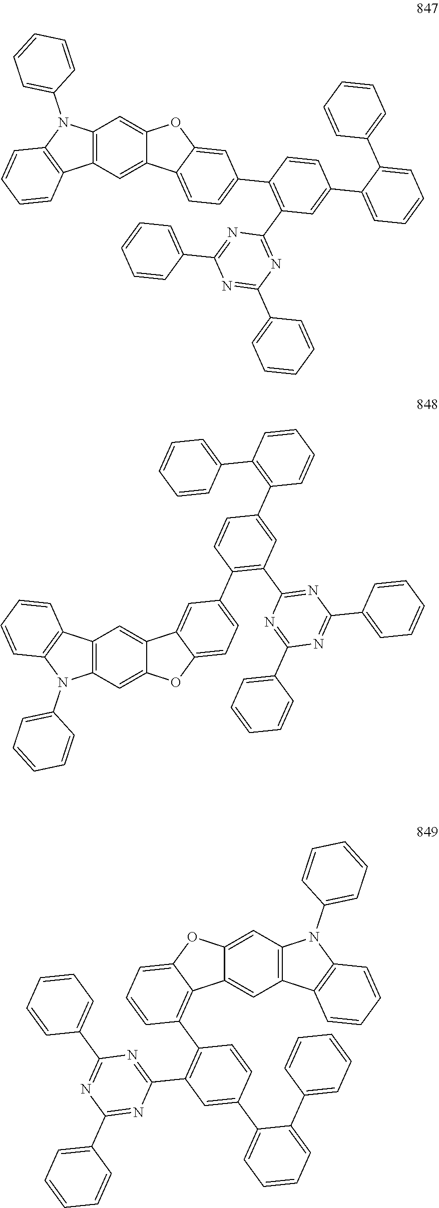 [SMILES: c1ccc(-c2nc(-c3ccccc3)nc(-c3cc(-c4ccccc4-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccccc4-c4ccccc4)ccc3-c3ccc4oc5cc6c(cc5c4c3)c3ccccc3n6-c3ccccc3)n2)cc1 ; c1ccc(-c2nc(-c3ccccc3)nc(-c3cc(-c4ccccc4-c4ccccc4)ccc3-c3cccc4oc5cc6c(cc5c34)c3ccccc3n6-c3ccccc3)n2)cc1]

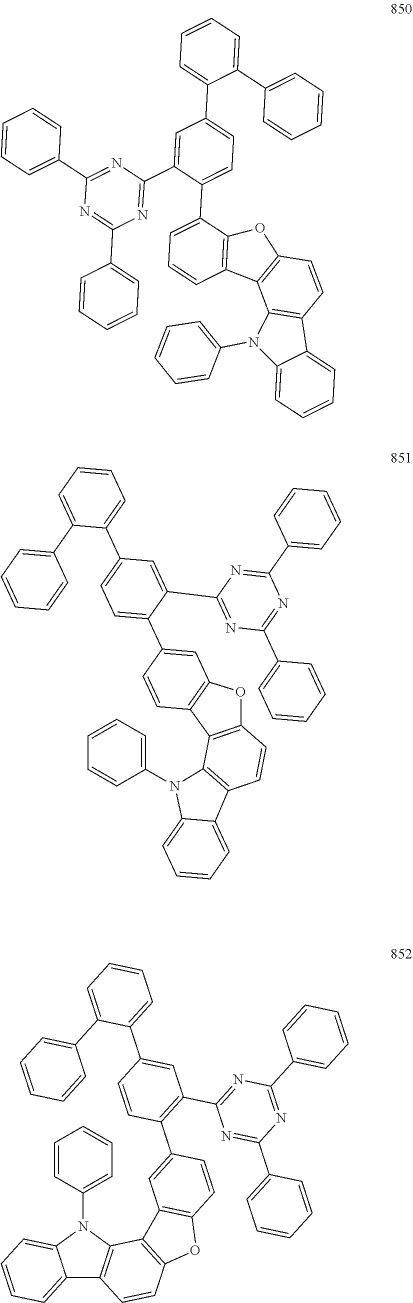 [SMILES: c1ccc(-c2nc(-c3ccccc3)nc(-c3cc(-c4ccccc4-c4ccccc4)ccc3-c3cccc4c3oc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4c(c3)oc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4oc5ccc6c7ccccc7n(-c7ccccc7)c6c5c4c3)n2)cc1]

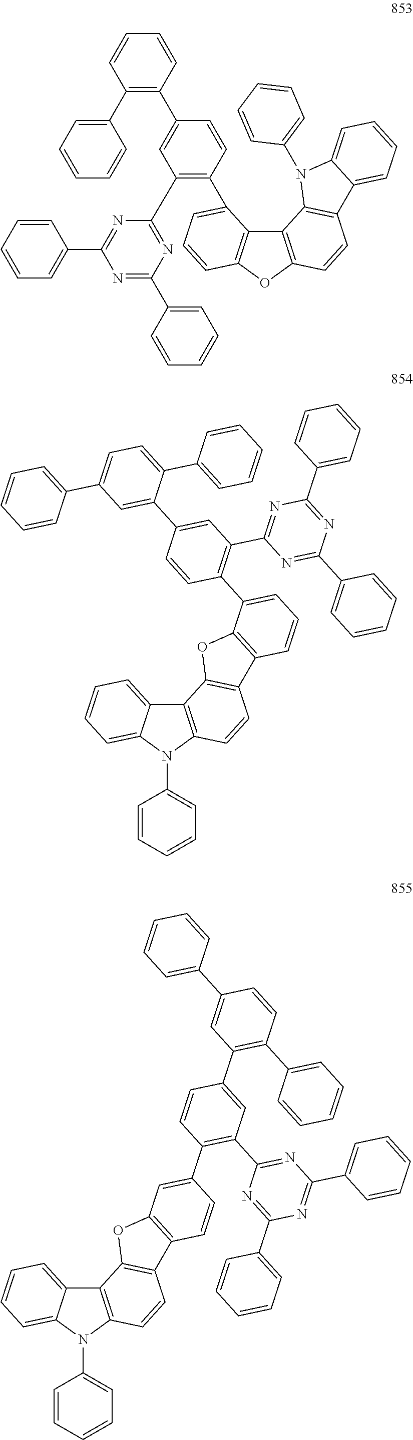 [SMILES: c1ccc(-c2nc(-c3ccccc3)nc(-c3cc(-c4ccccc4-c4ccccc4)ccc3-c3cccc4oc5ccc6c7ccccc7n(-c7ccccc7)c6c5c34)n2)cc1 ; c1ccc(-c2ccc(-c3ccccc3)c(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

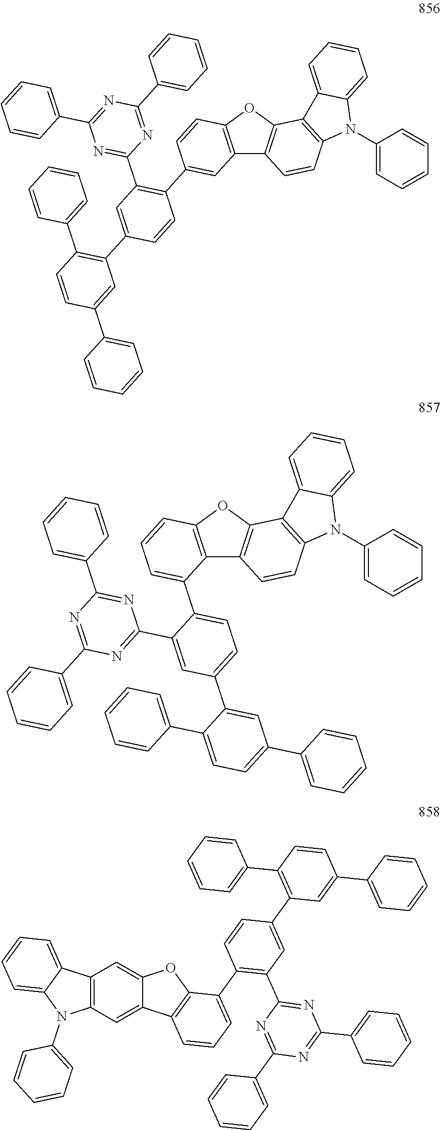 [SMILES: c1ccc(-c2ccc(-c3ccccc3)c(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

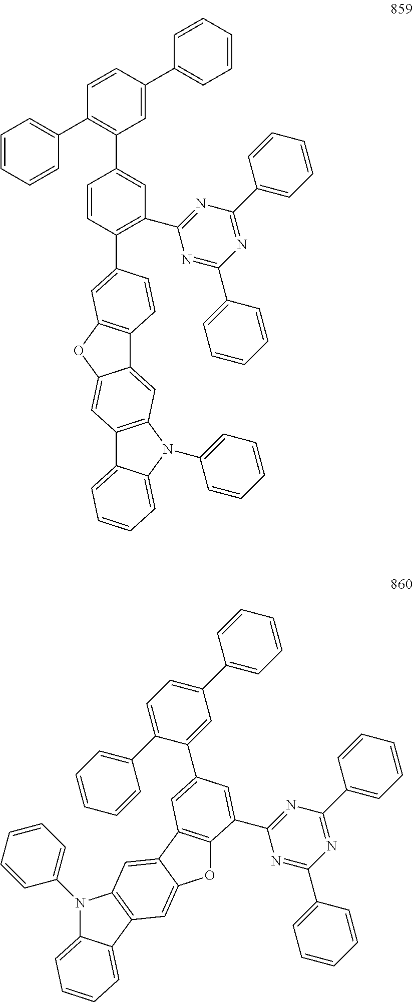 [SMILES: c1ccc(-c2ccc(-c3ccccc3)c(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cc(-c4nc(-c5ccccc5)nc(-c5ccccc5)n4)c4oc5cc6c7ccccc7n(-c7ccccc7)c6cc5c4c3)c2)cc1]

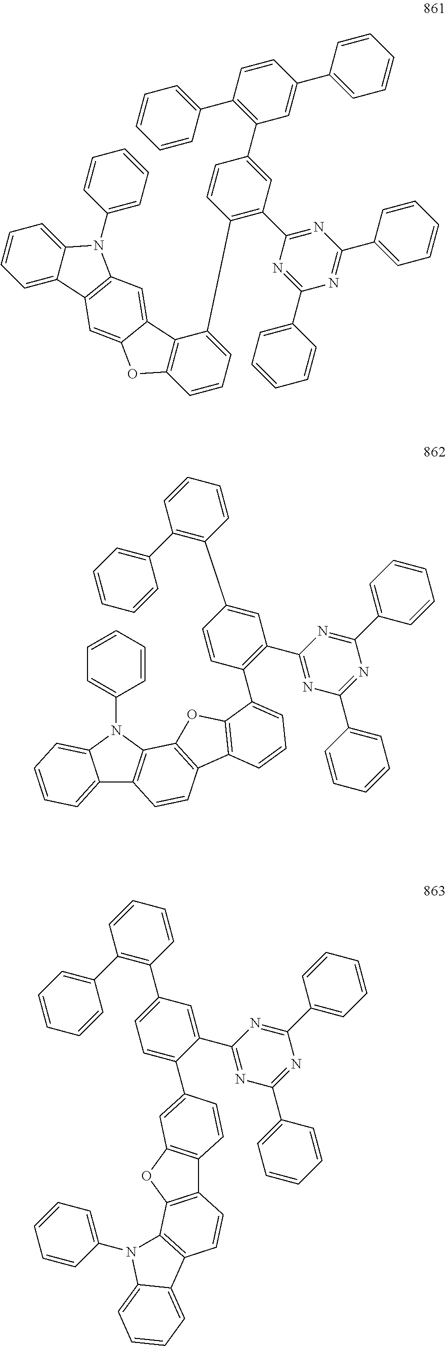 [SMILES: c1ccc(-c2ccc(-c3ccccc3)c(-c3ccc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)ccc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4c(c3)oc3c4ccc4c5ccccc5n(-c5ccccc5)c43)n2)cc1]

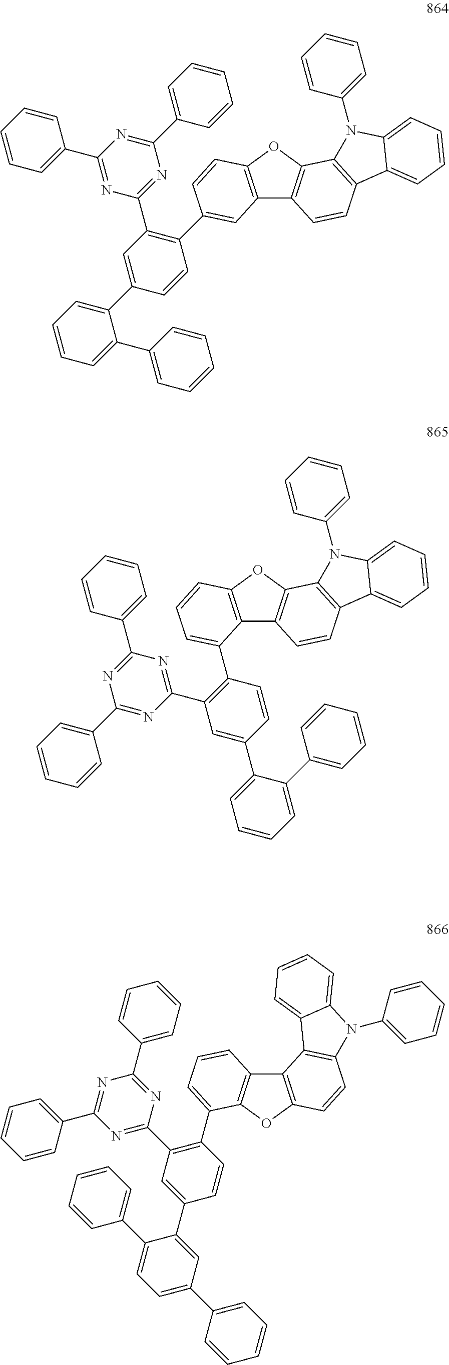 [SMILES: c1ccc(-c2nc(-c3ccccc3)nc(-c3cc(-c4ccccc4-c4ccccc4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)ccc3-c3cccc4oc5c(ccc6c7ccccc7n(-c7ccccc7)c65)c34)n2)cc1 ; c1ccc(-c2ccc(-c3ccccc3)c(-c3ccc(-c4cccc5c4oc4ccc6c(c7ccccc7n6-c6ccccc6)c45)c(-c4nc(-c5ccccc5)nc(-c5ccccc5)n4)c3)c2)cc1]

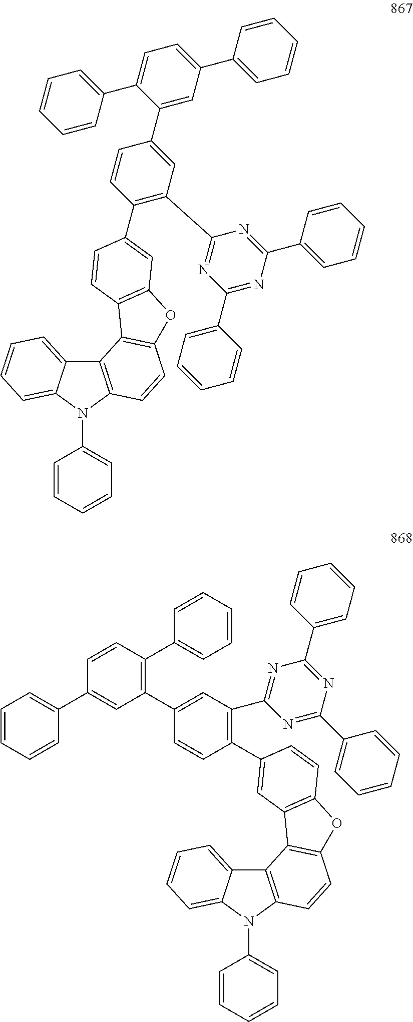 [SMILES: c1ccc(-c2ccc(-c3ccccc3)c(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1]

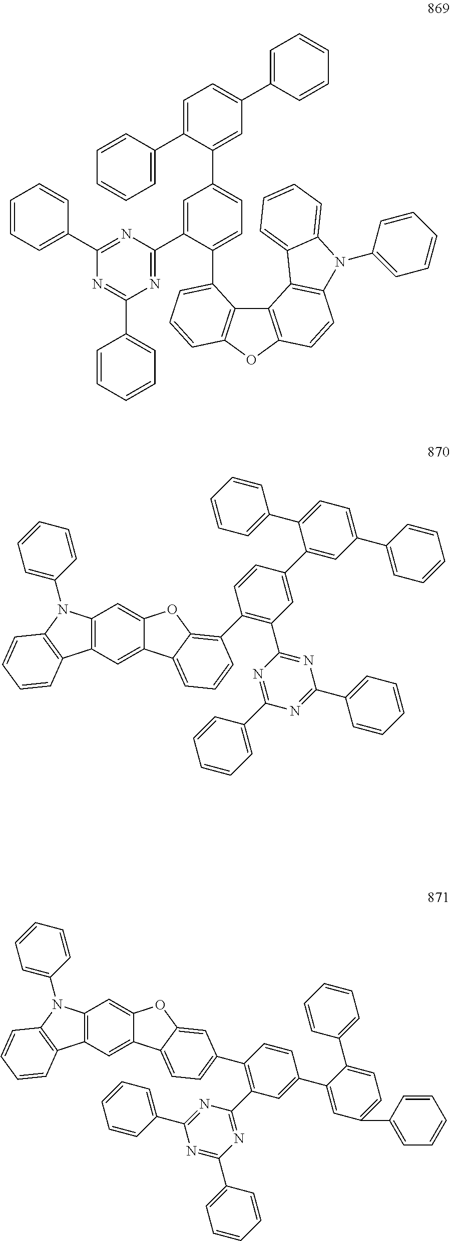 [SMILES: c1ccc(-c2ccc(-c3ccccc3)c(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

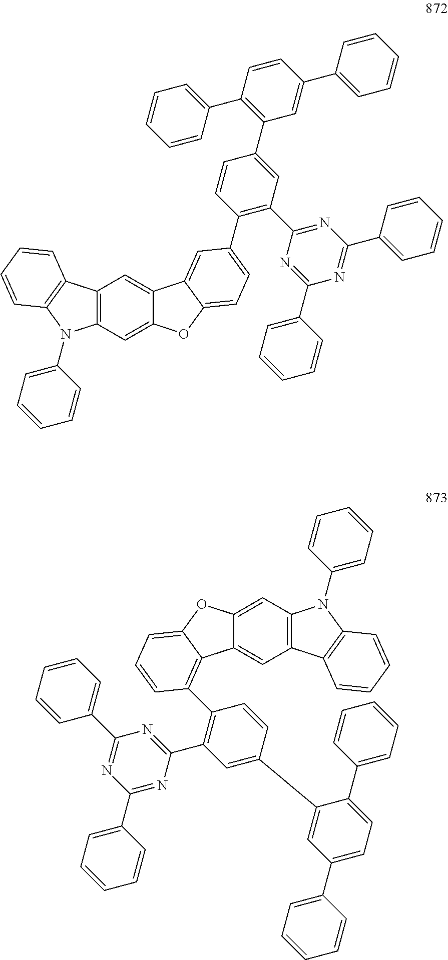 [SMILES: c1ccc(-c2ccc(-c3ccccc3)c(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5oc6cc7c(cc6c45)c4ccccc4n7-c4ccccc4)c(-c4nc(-c5ccccc5)nc(-c5ccccc5)n4)c3)c2)cc1]

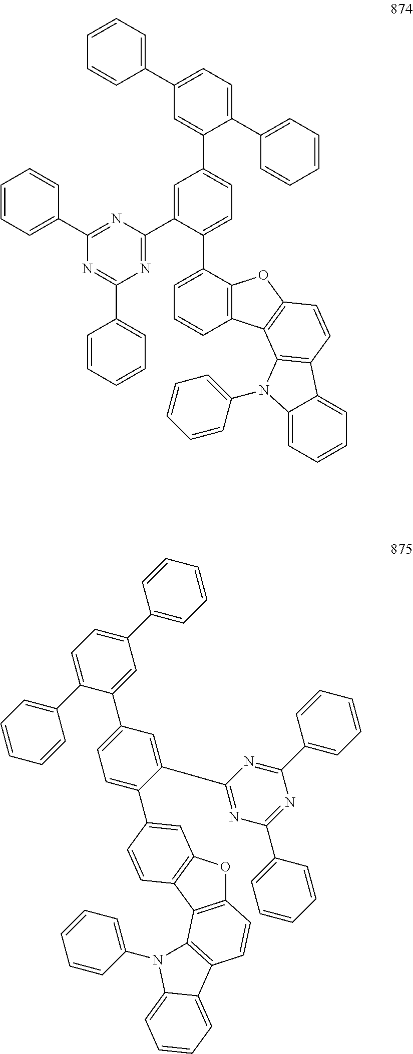 [SMILES: c1ccc(-c2ccc(-c3ccccc3)c(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1]

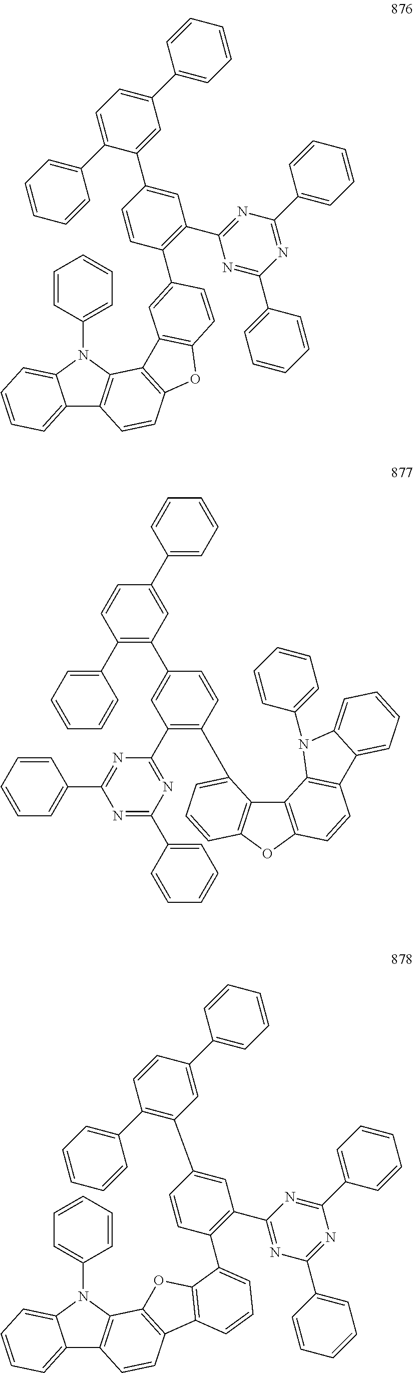 [SMILES: c1ccc(-c2ccc(-c3ccccc3)c(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3ccc(-c4cccc5c4oc4c5ccc5c6ccccc6n(-c6ccccc6)c54)c(-c4nc(-c5ccccc5)nc(-c5ccccc5)n4)c3)c2)cc1]

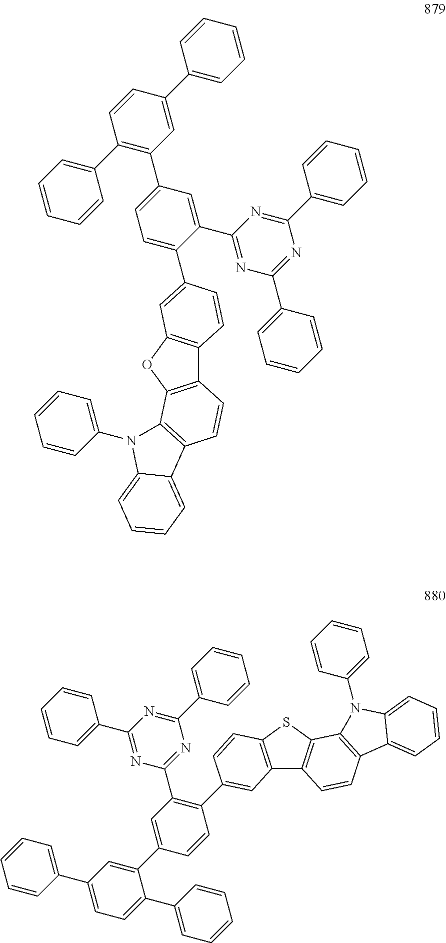 [SMILES: c1ccc(-c2ccc(-c3ccccc3)c(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1]

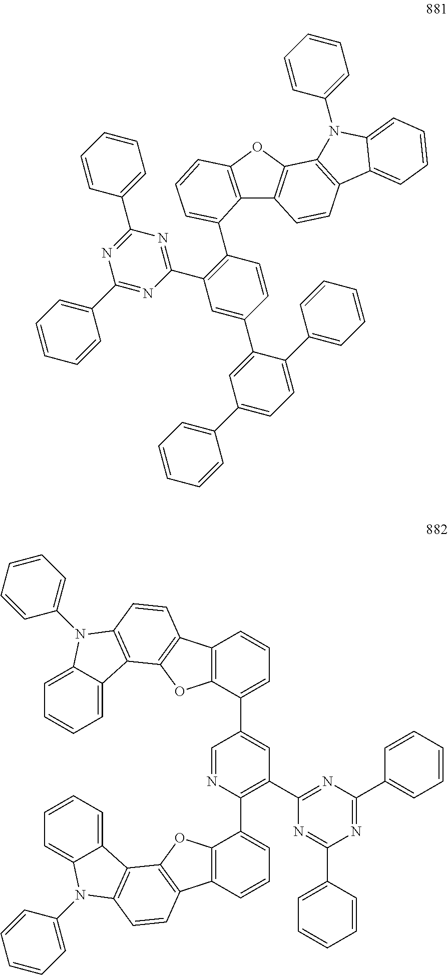 [SMILES: c1ccc(-c2ccc(-c3ccccc3)c(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4cccc5c4oc4c5ccc5c4c4ccccc4n5-c4ccccc4)cnc3-c3cccc4c3oc3c4ccc4c3c3ccccc3n4-c3ccccc3)n2)cc1]

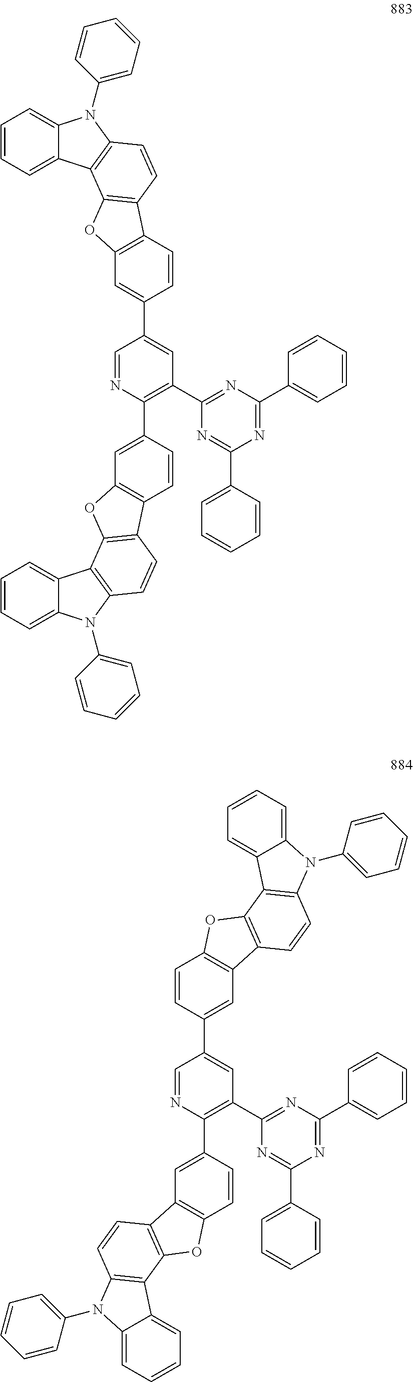 [SMILES: c1ccc(-c2nc(-c3ccccc3)nc(-c3cc(-c4ccc5c(c4)oc4c5ccc5c4c4ccccc4n5-c4ccccc4)cnc3-c3ccc4c(c3)oc3c4ccc4c3c3ccccc3n4-c3ccccc3)n2)cc1 ; c1ccc(-c2nc(-c3ccccc3)nc(-c3cc(-c4ccc5oc6c(ccc7c6c6ccccc6n7-c6ccccc6)c5c4)cnc3-c3ccc4oc5c(ccc6c5c5ccccc5n6-c5ccccc5)c4c3)n2)cc1]

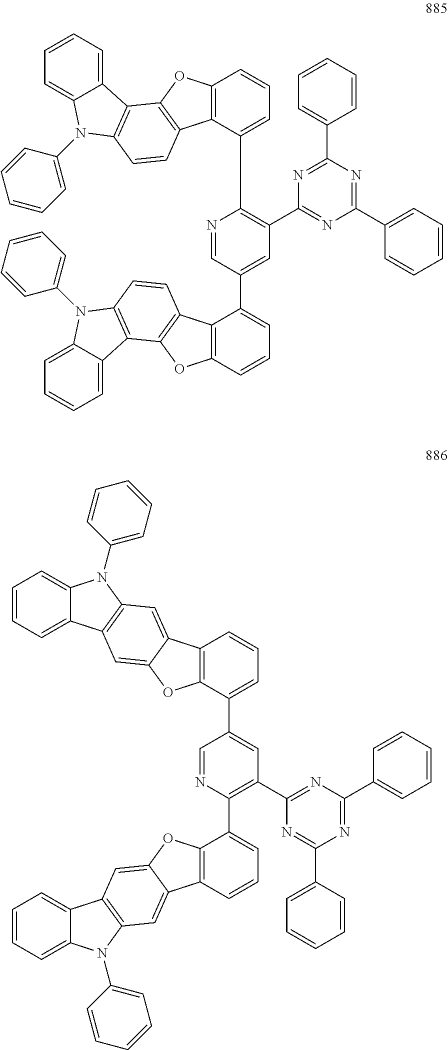 [SMILES: c1ccc(-c2nc(-c3ccccc3)nc(-c3cc(-c4cccc5oc6c(ccc7c6c6ccccc6n7-c6ccccc6)c45)cnc3-c3cccc4oc5c(ccc6c5c5ccccc5n6-c5ccccc5)c34)n2)cc1 ; c1ccc(-c2nc(-c3ccccc3)nc(-c3cc(-c4cccc5c4oc4cc6c7ccccc7n(-c7ccccc7)c6cc45)cnc3-c3cccc4c3oc3cc5c6ccccc6n(-c6ccccc6)c5cc34)n2)cc1]

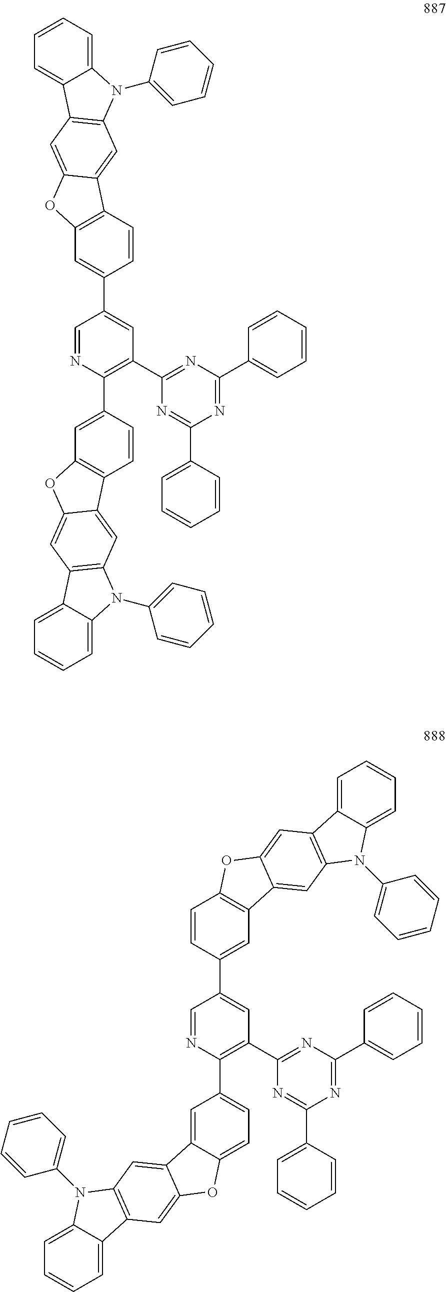 [SMILES: c1ccc(-c2nc(-c3ccccc3)nc(-c3cc(-c4ccc5c(c4)oc4cc6c7ccccc7n(-c7ccccc7)c6cc45)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccc5oc6cc7c8ccccc8n(-c8ccccc8)c7cc6c5c4)cnc3-c3ccc4oc5cc6c7ccccc7n(-c7ccccc7)c6cc5c4c3)n2)cc1]

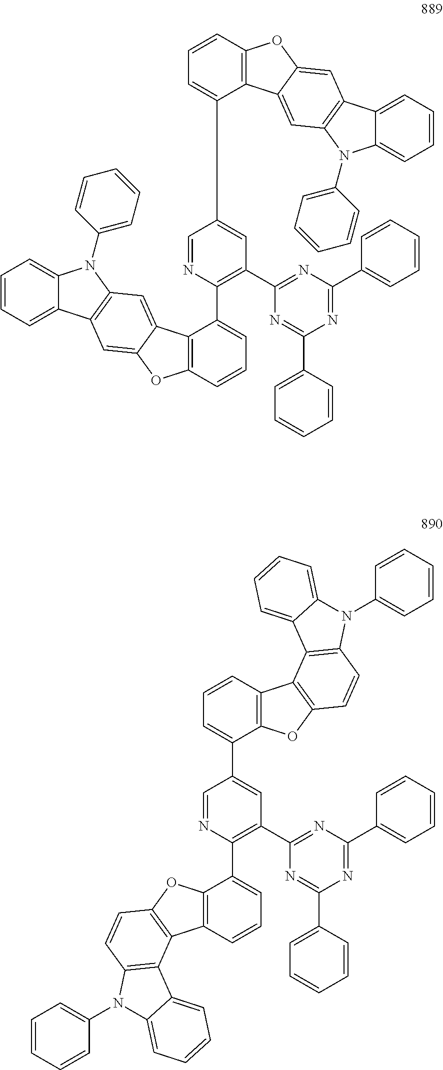 [SMILES: c1ccc(-c2nc(-c3ccccc3)nc(-c3cc(-c4cccc5oc6cc7c8ccccc8n(-c8ccccc8)c7cc6c45)cnc3-c3cccc4oc5cc6c7ccccc7n(-c7ccccc7)c6cc5c34)n2)cc1 ; c1ccc(-c2nc(-c3ccccc3)nc(-c3cc(-c4cccc5c4oc4ccc6c(c7ccccc7n6-c6ccccc6)c45)cnc3-c3cccc4c3oc3ccc5c(c6ccccc6n5-c5ccccc5)c34)n2)cc1]

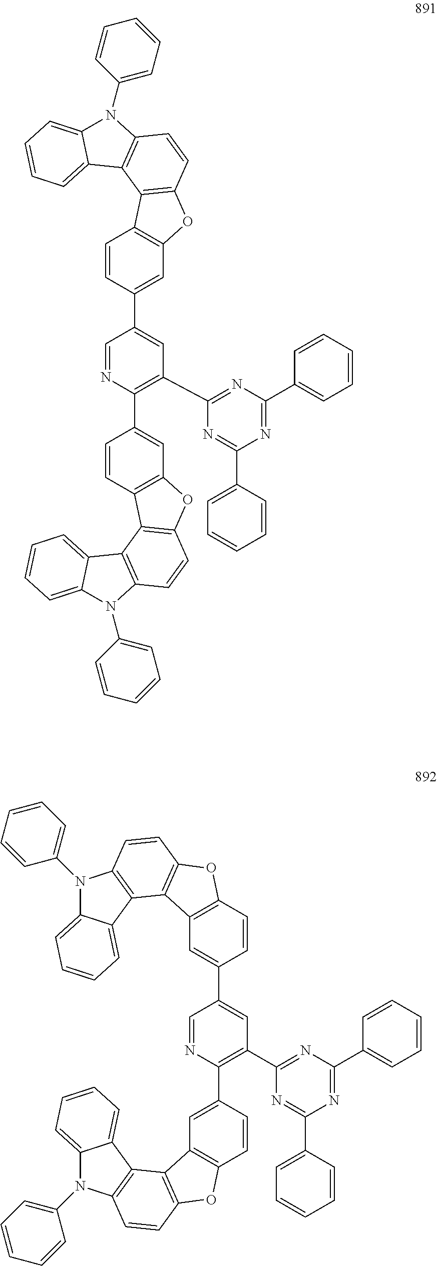 [SMILES: c1ccc(-c2nc(-c3ccccc3)nc(-c3cc(-c4ccc5c(c4)oc4ccc6c(c7ccccc7n6-c6ccccc6)c45)cnc3-c3ccc4c(c3)oc3ccc5c(c6ccccc6n5-c5ccccc5)c34)n2)cc1 ; c1ccc(-c2nc(-c3ccccc3)nc(-c3cc(-c4ccc5oc6ccc7c(c8ccccc8n7-c7ccccc7)c6c5c4)cnc3-c3ccc4oc5ccc6c(c7ccccc7n6-c6ccccc6)c5c4c3)n2)cc1]

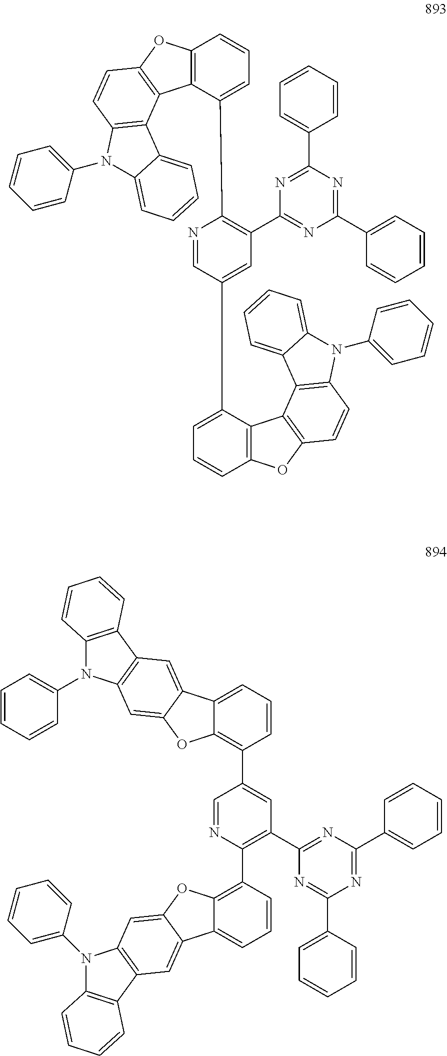 [SMILES: c1ccc(-c2nc(-c3ccccc3)nc(-c3cc(-c4cccc5oc6ccc7c(c8ccccc8n7-c7ccccc7)c6c45)cnc3-c3cccc4oc5ccc6c(c7ccccc7n6-c6ccccc6)c5c34)n2)cc1 ; c1ccc(-c2nc(-c3ccccc3)nc(-c3cc(-c4cccc5c4oc4cc6c(cc45)c4ccccc4n6-c4ccccc4)cnc3-c3cccc4c3oc3cc5c(cc34)c3ccccc3n5-c3ccccc3)n2)cc1]

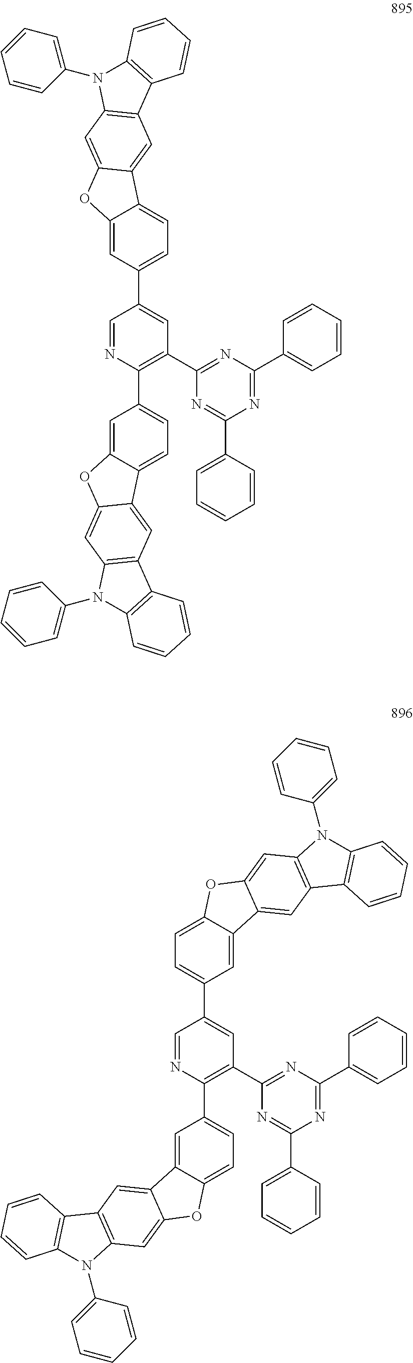 [SMILES: c1ccc(-c2nc(-c3ccccc3)nc(-c3cc(-c4ccc5c(c4)oc4cc6c(cc45)c4ccccc4n6-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccc5oc6cc7c(cc6c5c4)c4ccccc4n7-c4ccccc4)cnc3-c3ccc4oc5cc6c(cc5c4c3)c3ccccc3n6-c3ccccc3)n2)cc1]

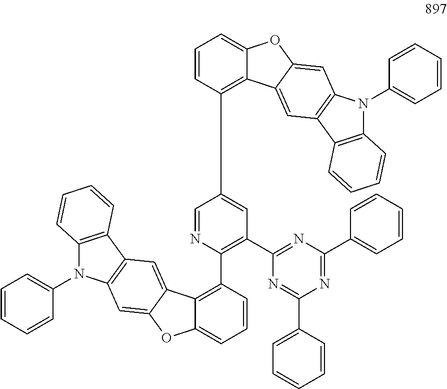 [SMILES: c1ccc(-c2nc(-c3ccccc3)nc(-c3cc(-c4cccc5oc6cc7c(cc6c45)c4ccccc4n7-c4ccccc4)cnc3-c3cccc4oc5cc6c(cc5c34)c3ccccc3n6-c3ccccc3)n2)cc1]

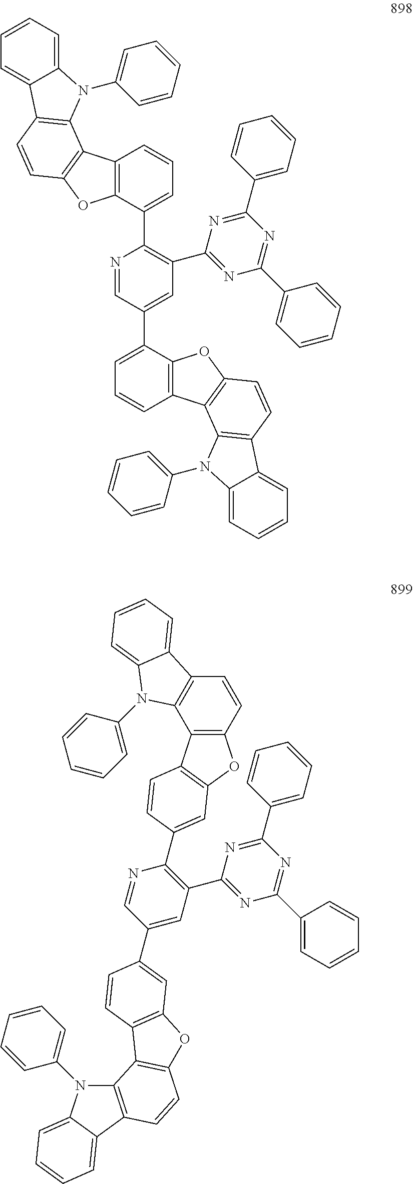 [SMILES: c1ccc(-c2nc(-c3ccccc3)nc(-c3cc(-c4cccc5c4oc4ccc6c7ccccc7n(-c7ccccc7)c6c45)cnc3-c3cccc4c3oc3ccc5c6ccccc6n(-c6ccccc6)c5c34)n2)cc1 ; c1ccc(-c2nc(-c3ccccc3)nc(-c3cc(-c4ccc5c(c4)oc4ccc6c7ccccc7n(-c7ccccc7)c6c45)cnc3-c3ccc4c(c3)oc3ccc5c6ccccc6n(-c6ccccc6)c5c34)n2)cc1]

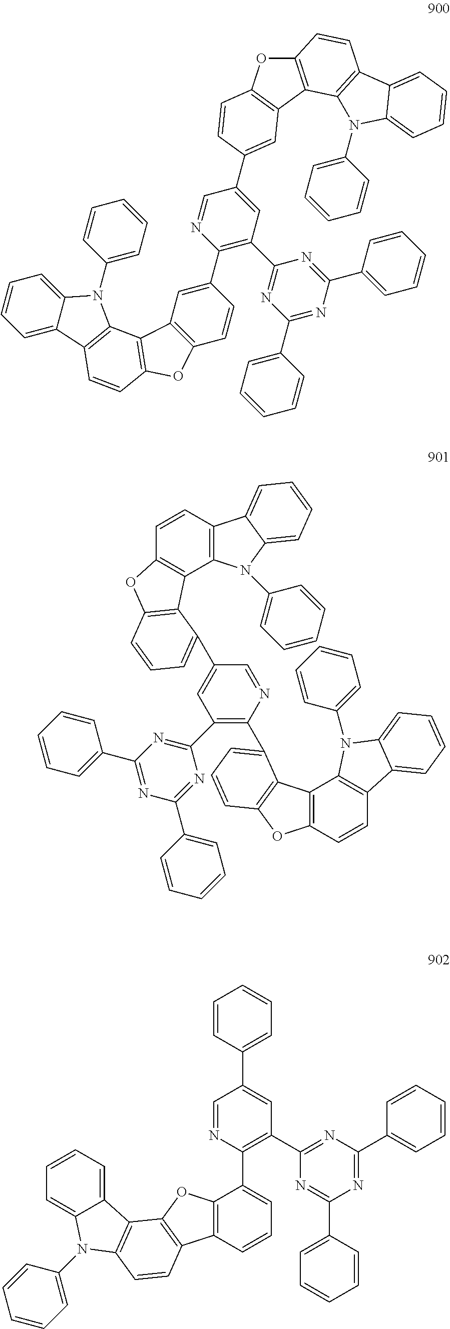 [SMILES: c1ccc(-c2nc(-c3ccccc3)nc(-c3cc(-c4ccc5oc6ccc7c8ccccc8n(-c8ccccc8)c7c6c5c4)cnc3-c3ccc4oc5ccc6c7ccccc7n(-c7ccccc7)c6c5c4c3)n2)cc1 ; c1ccc(-c2nc(-c3ccccc3)nc(-c3cc(-c4cccc5oc6ccc7c8ccccc8n(-c8ccccc8)c7c6c45)cnc3-c3cccc4oc5ccc6c7ccccc7n(-c7ccccc7)c6c5c34)n2)cc1 ; c1ccc(-c2cnc(-c3cccc4c3oc3c4ccc4c3c3ccccc3n4-c3ccccc3)c(-c3nc(-c4ccccc4)nc(-c4ccccc4)n3)c2)cc1]

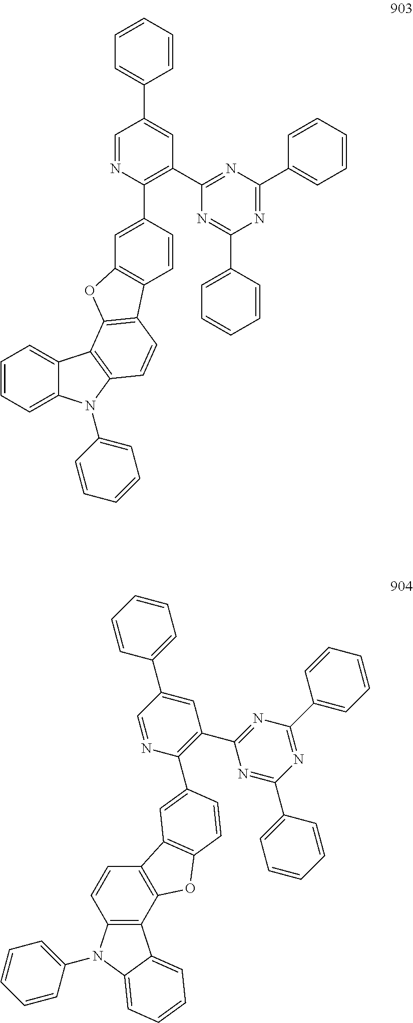 [SMILES: c1ccc(-c2cnc(-c3ccc4c(c3)oc3c4ccc4c3c3ccccc3n4-c3ccccc3)c(-c3nc(-c4ccccc4)nc(-c4ccccc4)n3)c2)cc1 ; c1ccc(-c2cnc(-c3ccc4oc5c(ccc6c5c5ccccc5n6-c5ccccc5)c4c3)c(-c3nc(-c4ccccc4)nc(-c4ccccc4)n3)c2)cc1]

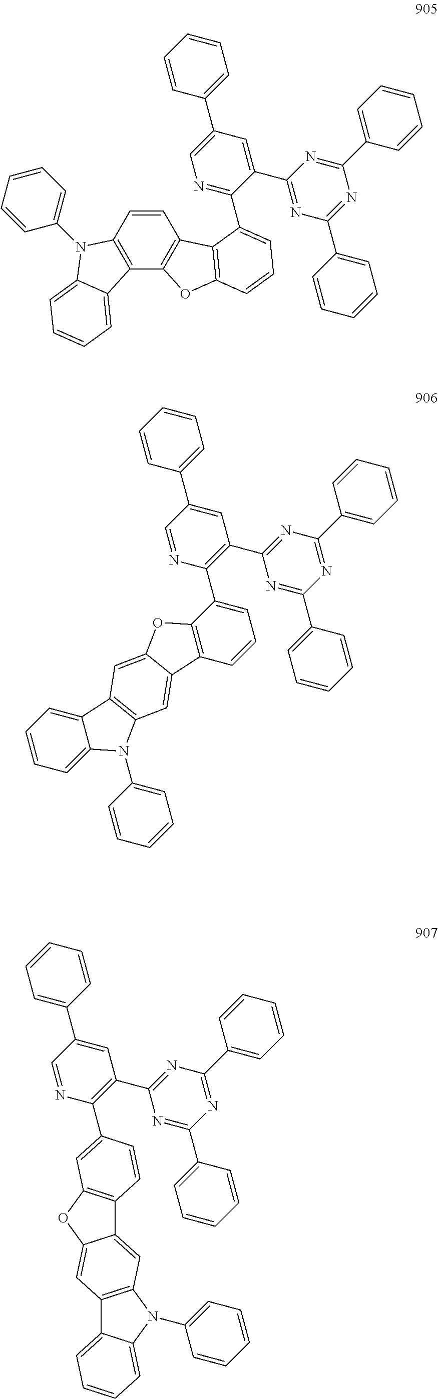 [SMILES: c1ccc(-c2cnc(-c3cccc4oc5c(ccc6c5c5ccccc5n6-c5ccccc5)c34)c(-c3nc(-c4ccccc4)nc(-c4ccccc4)n3)c2)cc1 ; c1ccc(-c2cnc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4c(c3)oc3cc5c6ccccc6n(-c6ccccc6)c5cc34)c(-c3nc(-c4ccccc4)nc(-c4ccccc4)n3)c2)cc1]

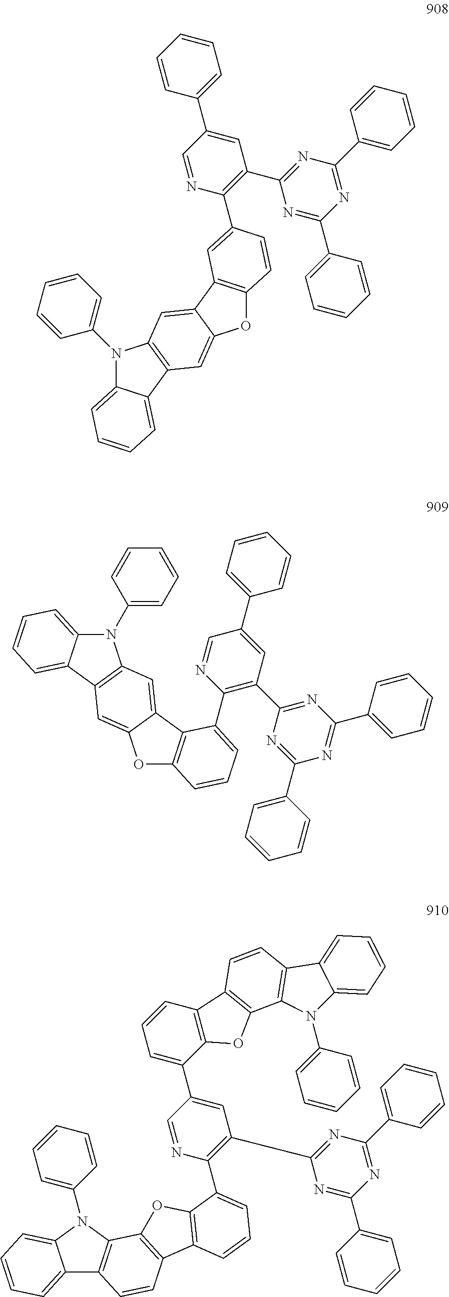 [SMILES: c1ccc(-c2cnc(-c3ccc4oc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4oc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4oc4c5ccc5c6ccccc6n(-c6ccccc6)c54)cnc3-c3cccc4c3oc3c4ccc4c5ccccc5n(-c5ccccc5)c43)n2)cc1]

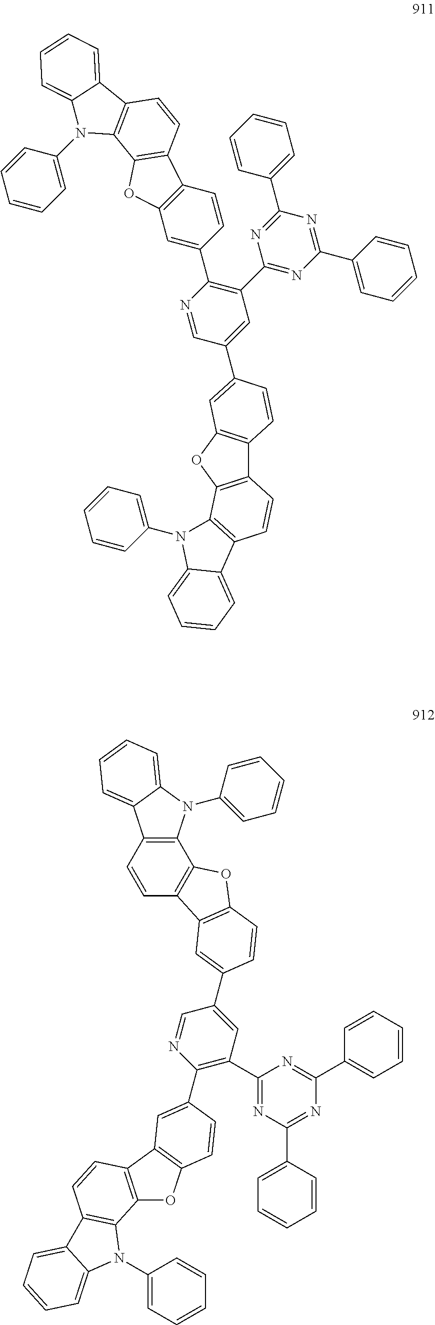 [SMILES: c1ccc(-c2nc(-c3ccccc3)nc(-c3cc(-c4ccc5c(c4)oc4c5ccc5c6ccccc6n(-c6ccccc6)c54)cnc3-c3ccc4c(c3)oc3c4ccc4c5ccccc5n(-c5ccccc5)c43)n2)cc1 ; c1ccc(-c2nc(-c3ccccc3)nc(-c3cc(-c4ccc5oc6c(ccc7c8ccccc8n(-c8ccccc8)c76)c5c4)cnc3-c3ccc4oc5c(ccc6c7ccccc7n(-c7ccccc7)c65)c4c3)n2)cc1]

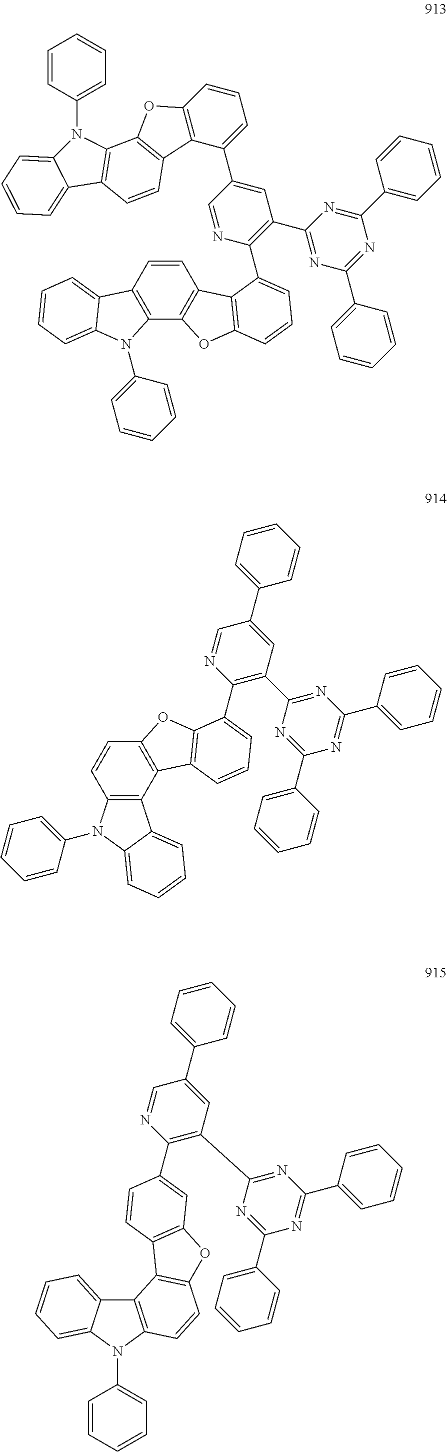 [SMILES: c1ccc(-c2nc(-c3ccccc3)nc(-c3cc(-c4cccc5oc6c(ccc7c8ccccc8n(-c8ccccc8)c76)c45)cnc3-c3cccc4oc5c(ccc6c7ccccc7n(-c7ccccc7)c65)c34)n2)cc1 ; c1ccc(-c2cnc(-c3cccc4c3oc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)oc3ccc5c(c6ccccc6n5-c5ccccc5)c34)c(-c3nc(-c4ccccc4)nc(-c4ccccc4)n3)c2)cc1]

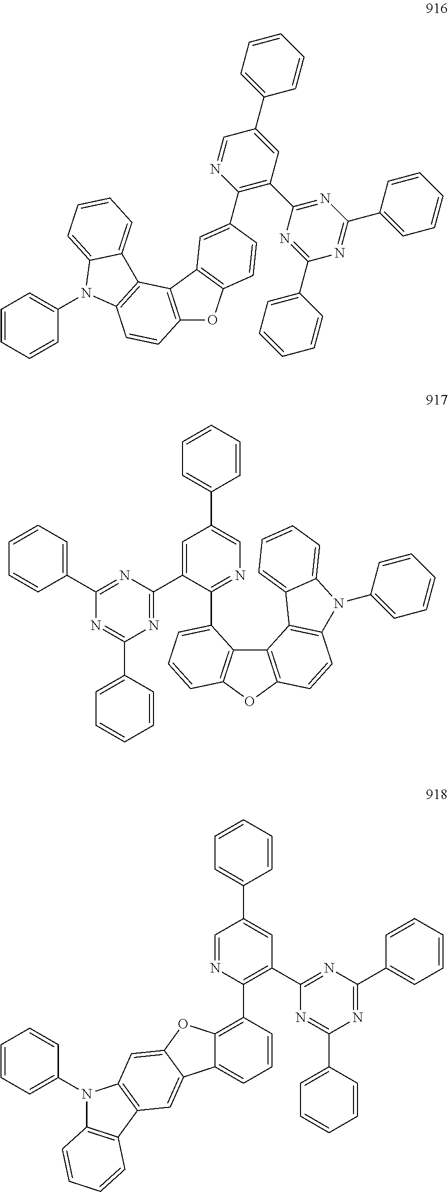 [SMILES: c1ccc(-c2cnc(-c3ccc4oc5ccc6c(c7ccccc7n6-c6ccccc6)c5c4c3)c(-c3nc(-c4ccccc4)nc(-c4ccccc4)n3)c2)cc1 ; c1ccc(-c2cnc(-c3cccc4oc5ccc6c(c7ccccc7n6-c6ccccc6)c5c34)c(-c3nc(-c4ccccc4)nc(-c4ccccc4)n3)c2)cc1 ; c1ccc(-c2cnc(-c3cccc4c3oc3cc5c(cc34)c3ccccc3n5-c3ccccc3)c(-c3nc(-c4ccccc4)nc(-c4ccccc4)n3)c2)cc1]

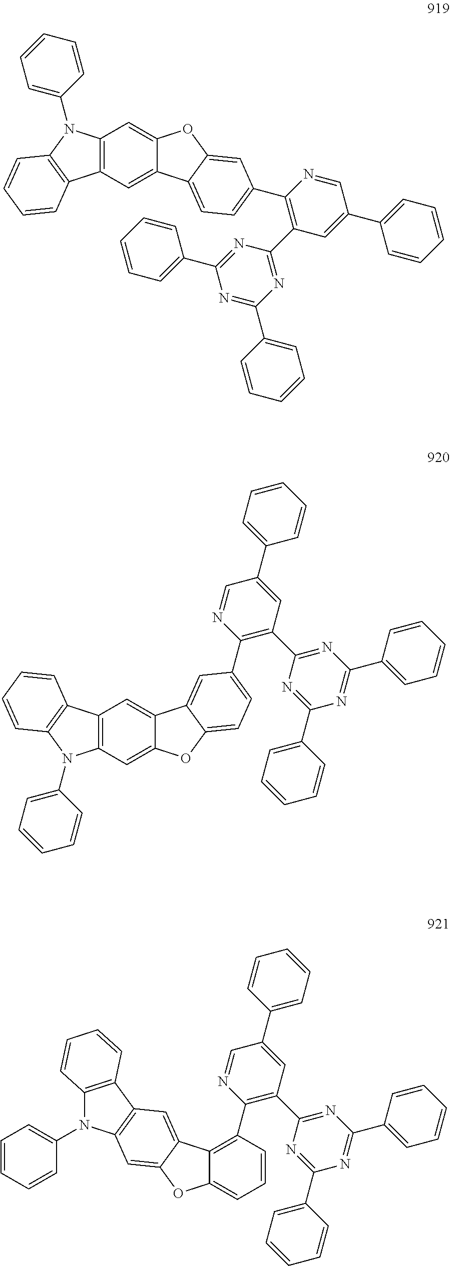 [SMILES: c1ccc(-c2cnc(-c3ccc4c(c3)oc3cc5c(cc34)c3ccccc3n5-c3ccccc3)c(-c3nc(-c4ccccc4)nc(-c4ccccc4)n3)c2)cc1 ; c1ccc(-c2cnc(-c3ccc4oc5cc6c(cc5c4c3)c3ccccc3n6-c3ccccc3)c(-c3nc(-c4ccccc4)nc(-c4ccccc4)n3)c2)cc1 ; c1ccc(-c2cnc(-c3cccc4oc5cc6c(cc5c34)c3ccccc3n6-c3ccccc3)c(-c3nc(-c4ccccc4)nc(-c4ccccc4)n3)c2)cc1]

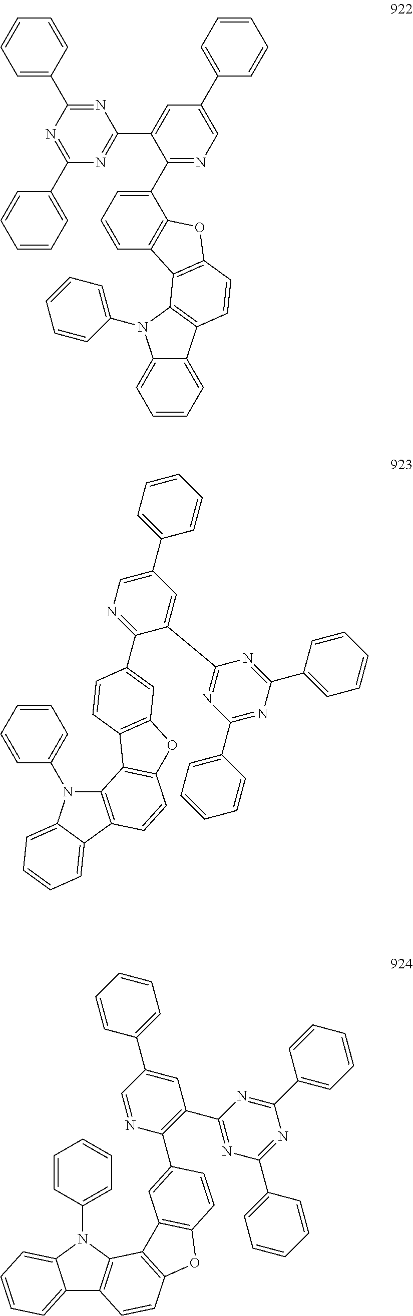 [SMILES: c1ccc(-c2cnc(-c3cccc4c3oc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4c(c3)oc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4oc5ccc6c7ccccc7n(-c7ccccc7)c6c5c4c3)c(-c3nc(-c4ccccc4)nc(-c4ccccc4)n3)c2)cc1]

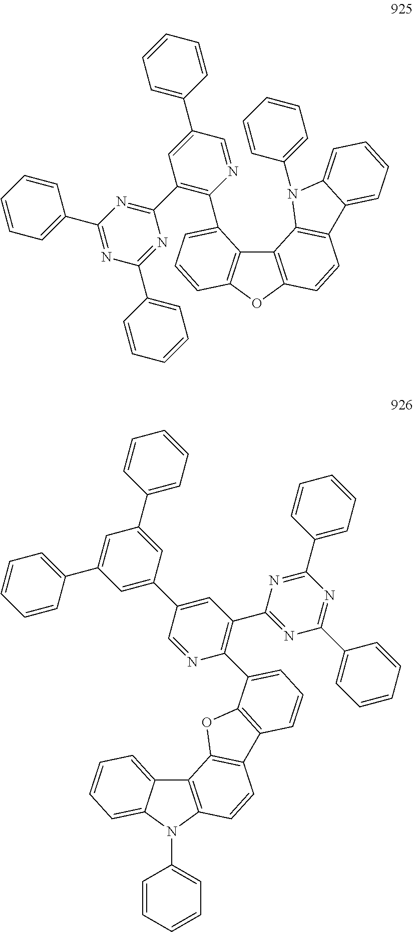 [SMILES: c1ccc(-c2cnc(-c3cccc4oc5ccc6c7ccccc7n(-c7ccccc7)c6c5c34)c(-c3nc(-c4ccccc4)nc(-c4ccccc4)n3)c2)cc1 ; c1ccc(-c2cc(-c3ccccc3)cc(-c3cnc(-c4cccc5c4oc4c5ccc5c4c4ccccc4n5-c4ccccc4)c(-c4nc(-c5ccccc5)nc(-c5ccccc5)n4)c3)c2)cc1]

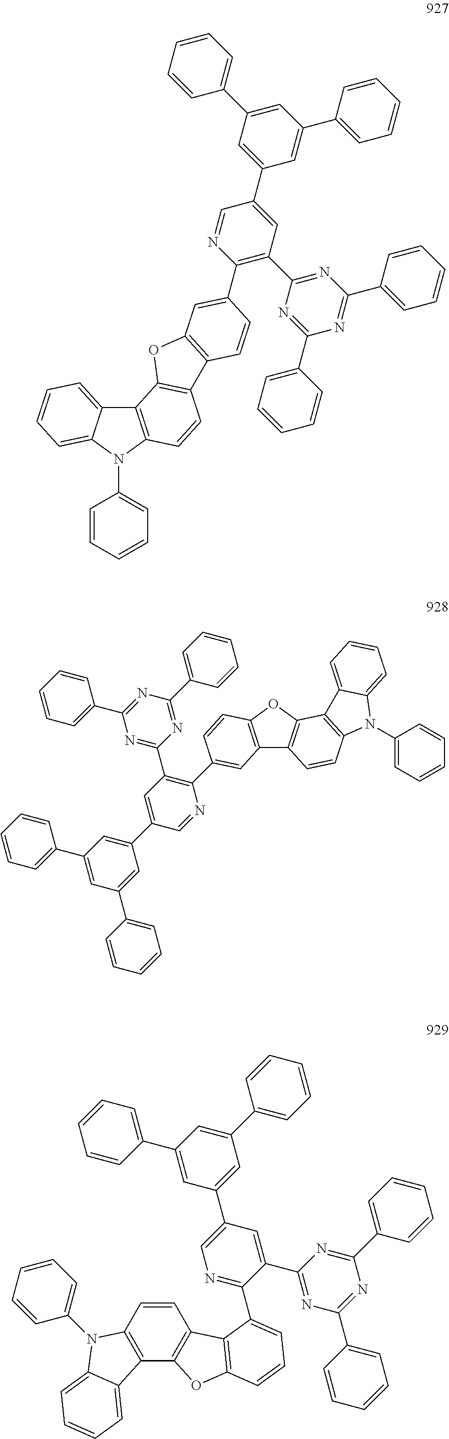 [SMILES: c1ccc(-c2cc(-c3ccccc3)cc(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

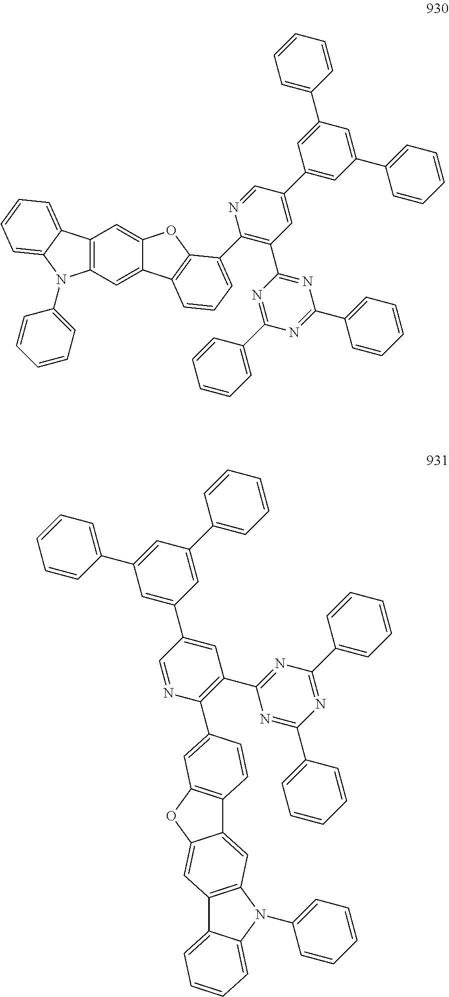 [SMILES: c1ccc(-c2cc(-c3ccccc3)cc(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1]

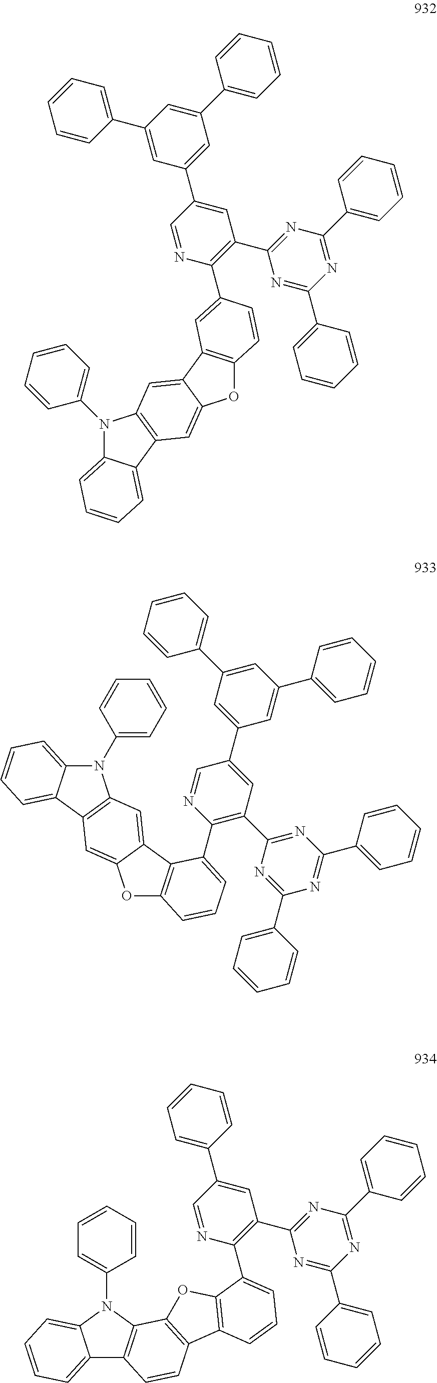 [SMILES: c1ccc(-c2cc(-c3ccccc3)cc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2cnc(-c3cccc4c3oc3c4ccc4c5ccccc5n(-c5ccccc5)c43)c(-c3nc(-c4ccccc4)nc(-c4ccccc4)n3)c2)cc1]

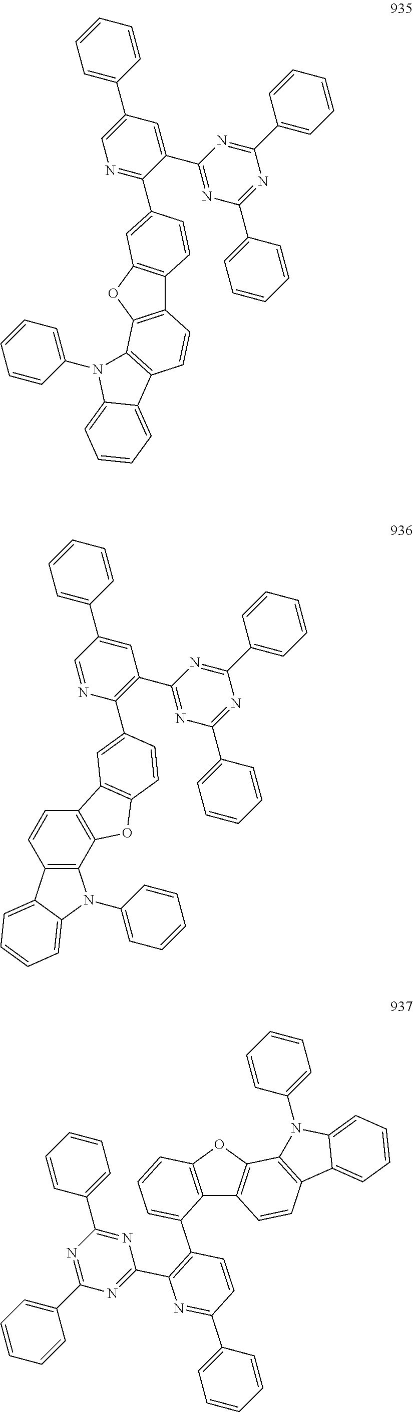 [SMILES: c1ccc(-c2cnc(-c3ccc4c(c3)oc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c7ccccc7n(-c7ccccc7)c65)c34)c(-c3nc(-c4ccccc4)nc(-c4ccccc4)n3)n2)cc1]

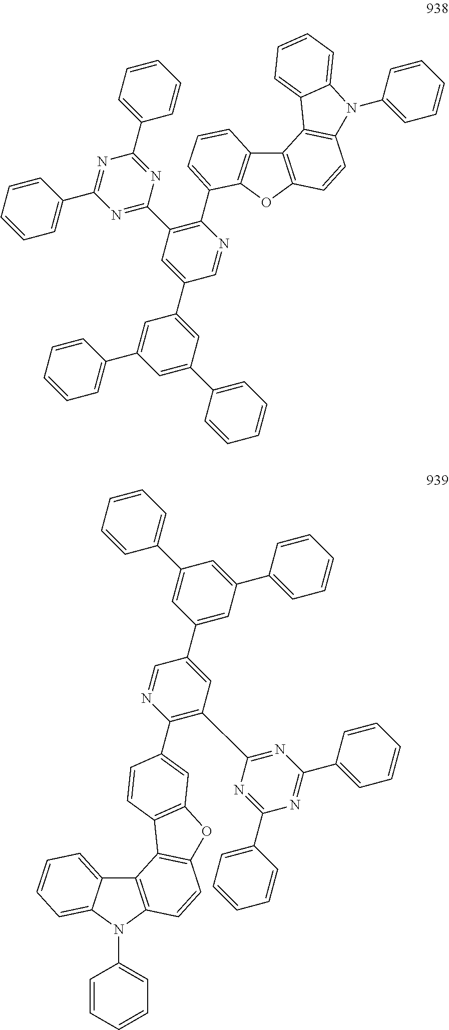 [SMILES: c1ccc(-c2cc(-c3ccccc3)cc(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1]

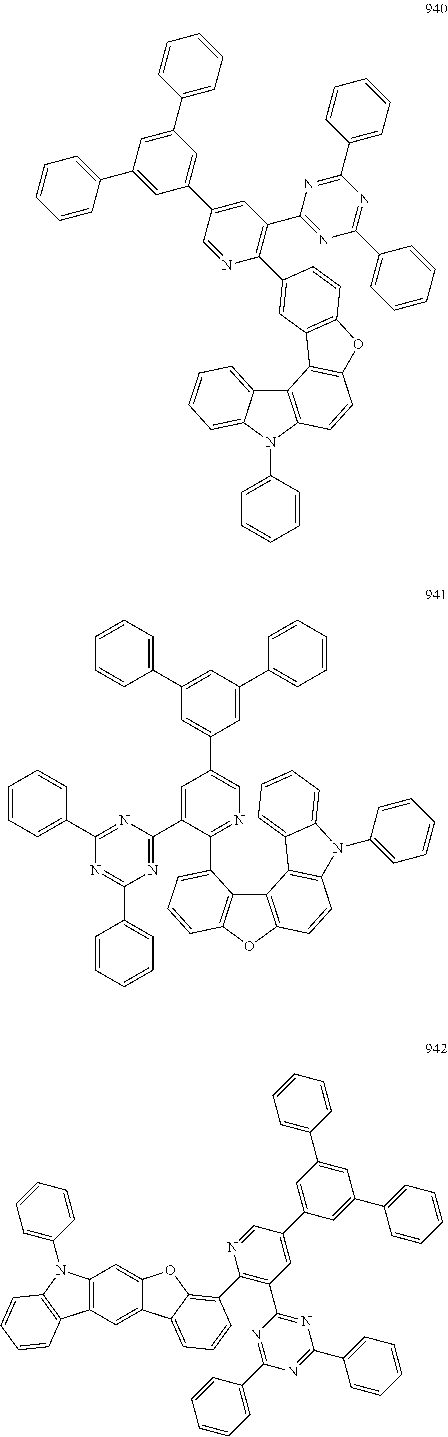 [SMILES: c1ccc(-c2cc(-c3ccccc3)cc(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4cccc5c4oc4cc6c(cc45)c4ccccc4n6-c4ccccc4)c(-c4nc(-c5ccccc5)nc(-c5ccccc5)n4)c3)c2)cc1]

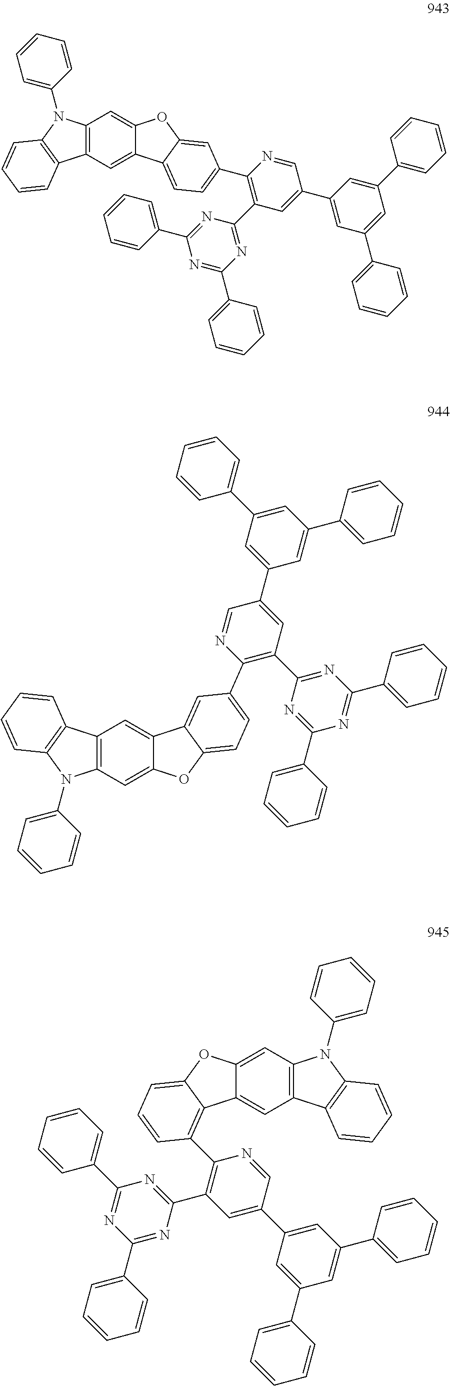 [SMILES: c1ccc(-c2cc(-c3ccccc3)cc(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3cnc(-c4cccc5oc6cc7c(cc6c45)c4ccccc4n7-c4ccccc4)c(-c4nc(-c5ccccc5)nc(-c5ccccc5)n4)c3)c2)cc1]

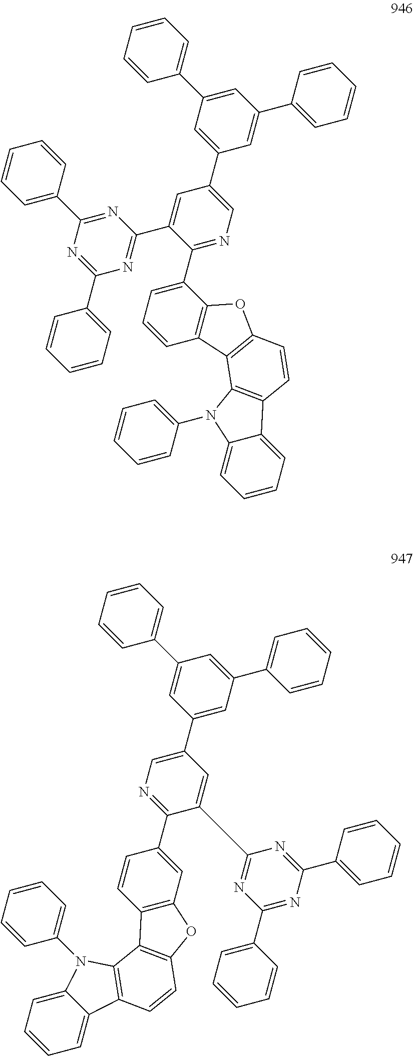 [SMILES: c1ccc(-c2cc(-c3ccccc3)cc(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1]

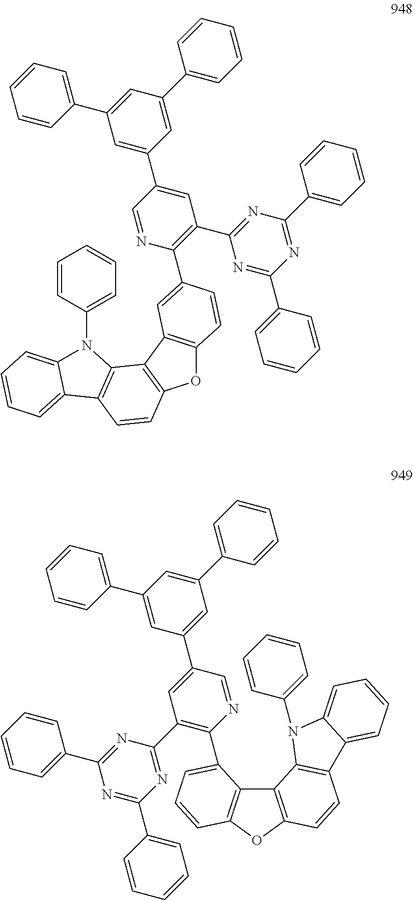 [SMILES: c1ccc(-c2cc(-c3ccccc3)cc(-c3cnc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

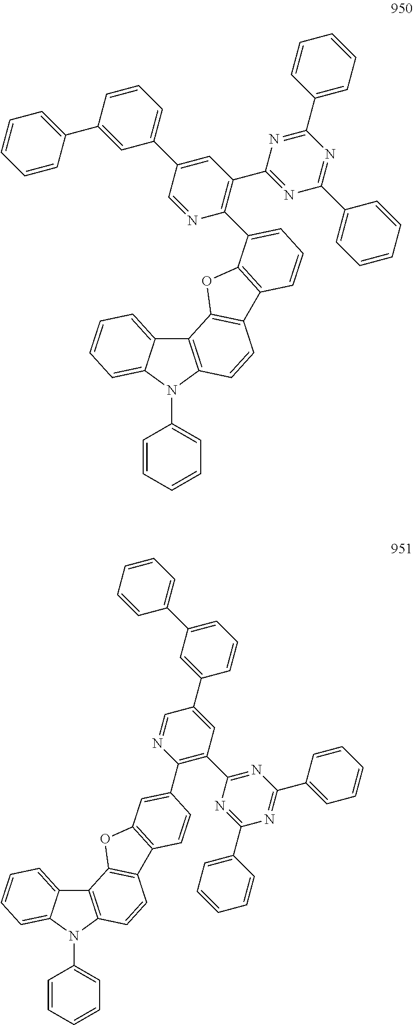 [SMILES: c1ccc(-c2cccc(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cccc(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1]

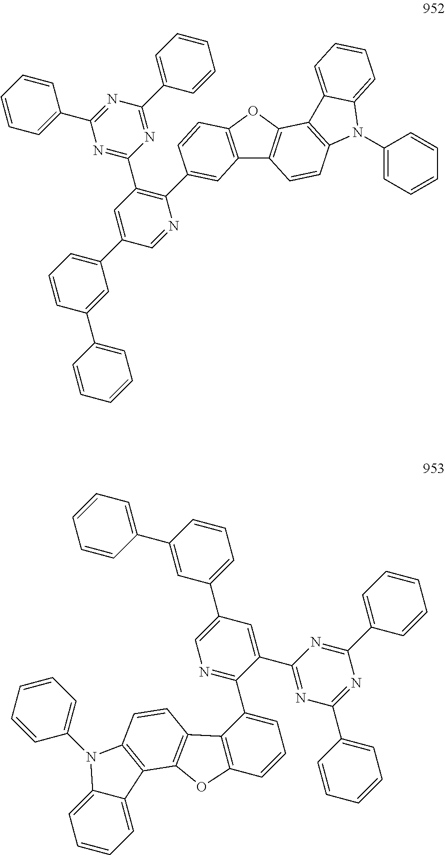 [SMILES: c1ccc(-c2cccc(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

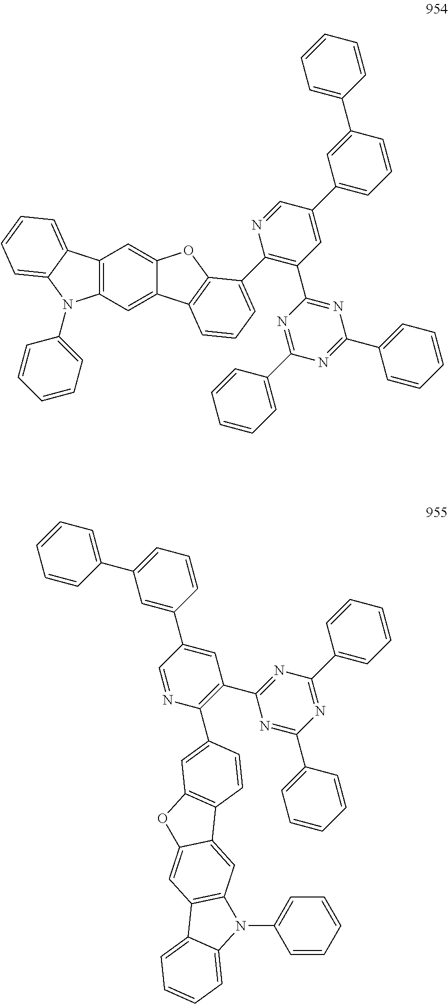 [SMILES: c1ccc(-c2cccc(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1]

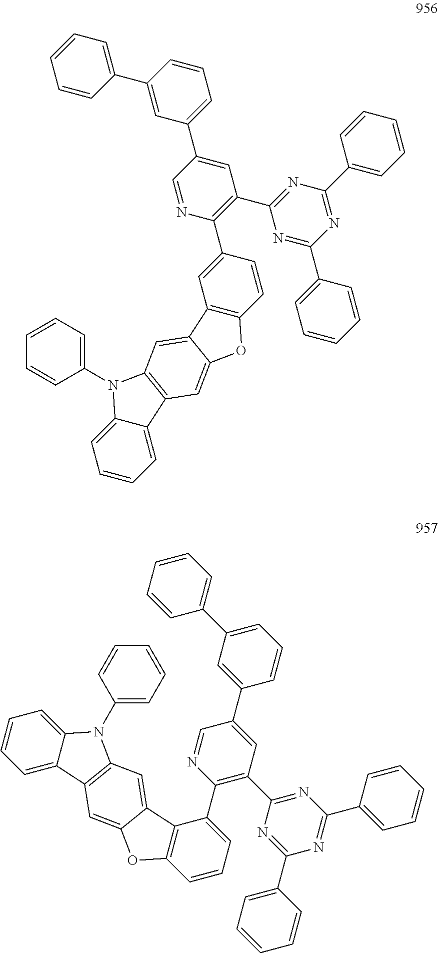 [SMILES: c1ccc(-c2cccc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

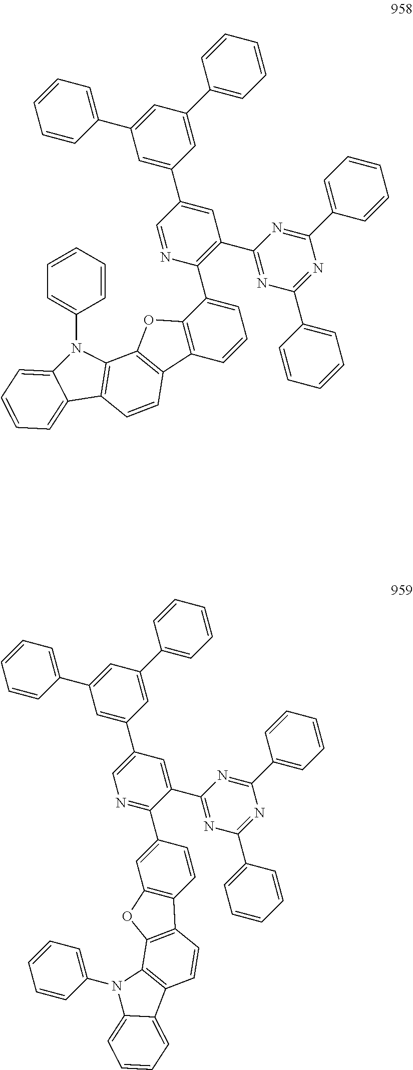 [SMILES: c1ccc(-c2cc(-c3ccccc3)cc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1]

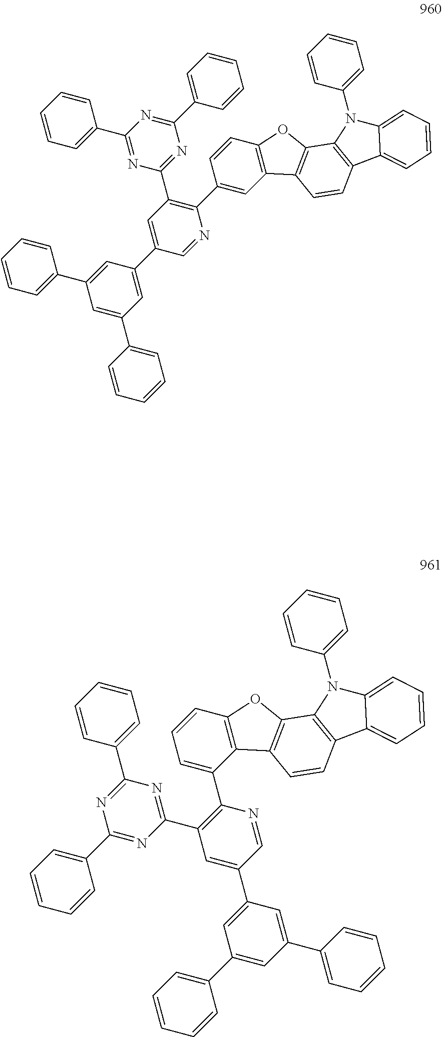 [SMILES: c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

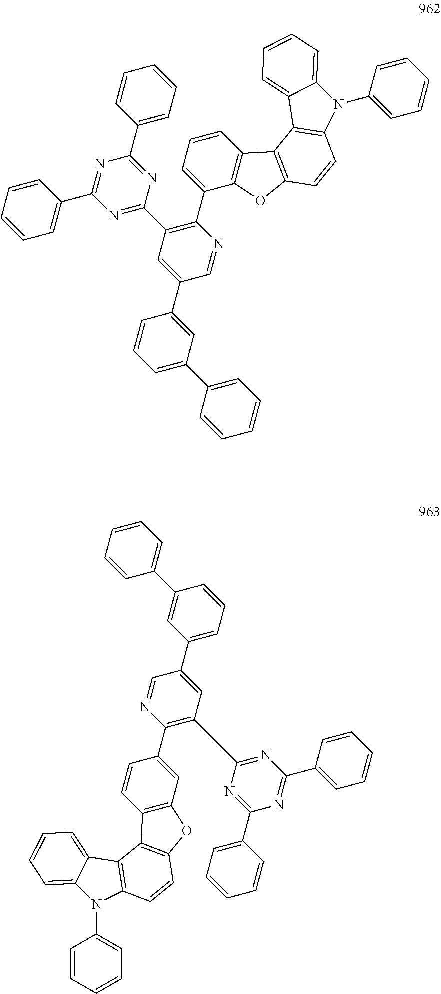 [SMILES: c1ccc(-c2cccc(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1]

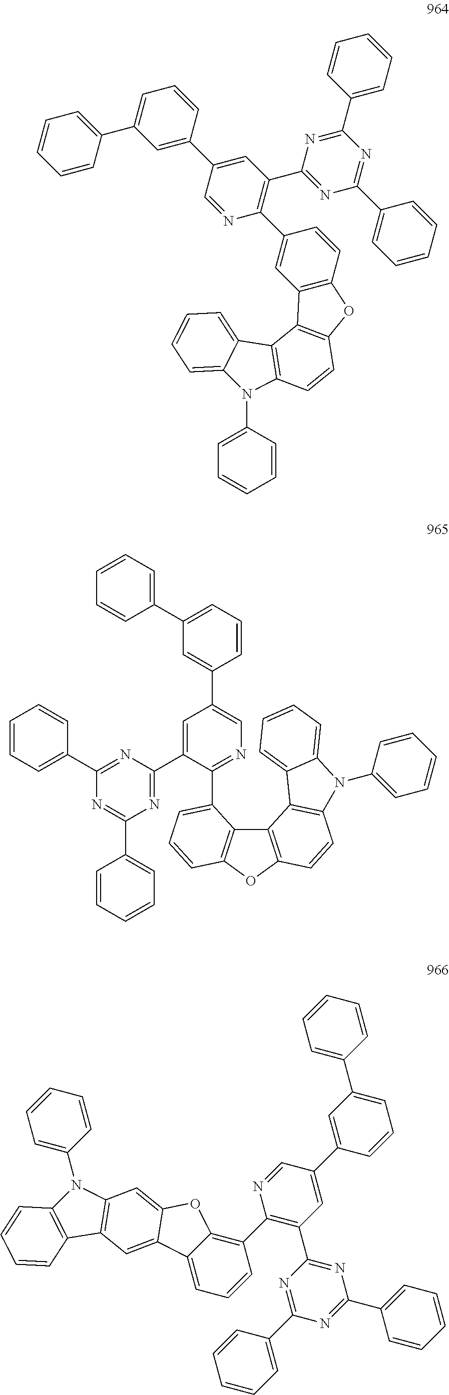 [SMILES: c1ccc(-c2cccc(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3cnc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3cnc(-c4cccc5c4oc4cc6c(cc45)c4ccccc4n6-c4ccccc4)c(-c4nc(-c5ccccc5)nc(-c5ccccc5)n4)c3)c2)cc1]

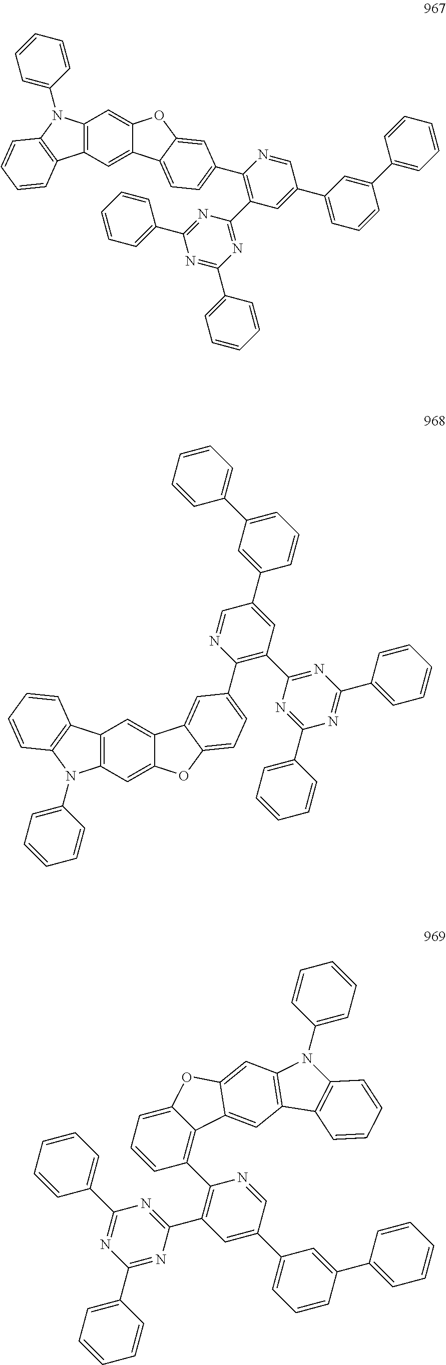 [SMILES: c1ccc(-c2cccc(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3cnc(-c4cccc5oc6cc7c(cc6c45)c4ccccc4n7-c4ccccc4)c(-c4nc(-c5ccccc5)nc(-c5ccccc5)n4)c3)c2)cc1]

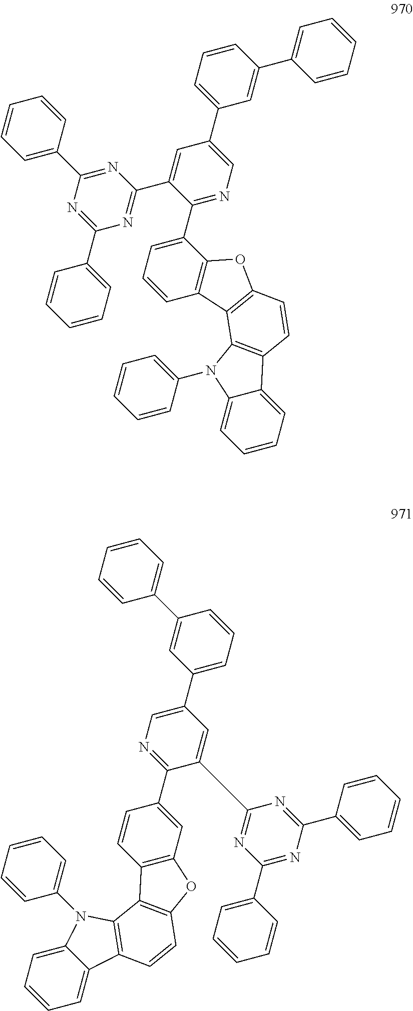 [SMILES: c1ccc(-c2cccc(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1]

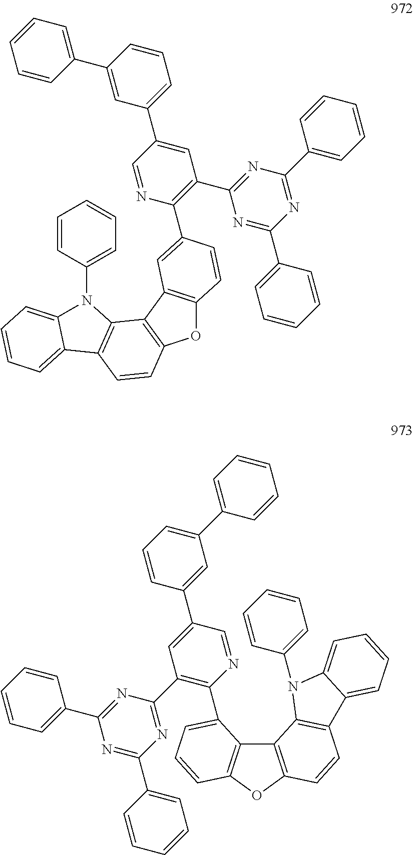 [SMILES: c1ccc(-c2cccc(-c3cnc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

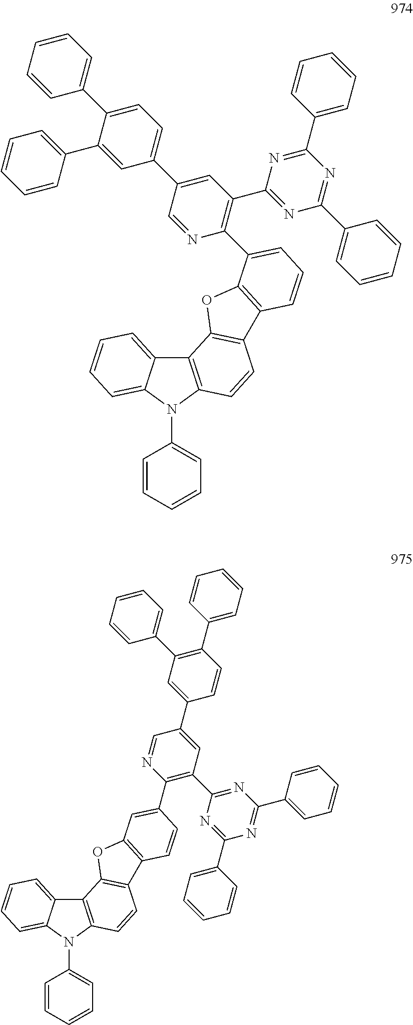 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)oc3c4ccc4c3c3ccccc3n4-c3ccccc3)n2)cc1]

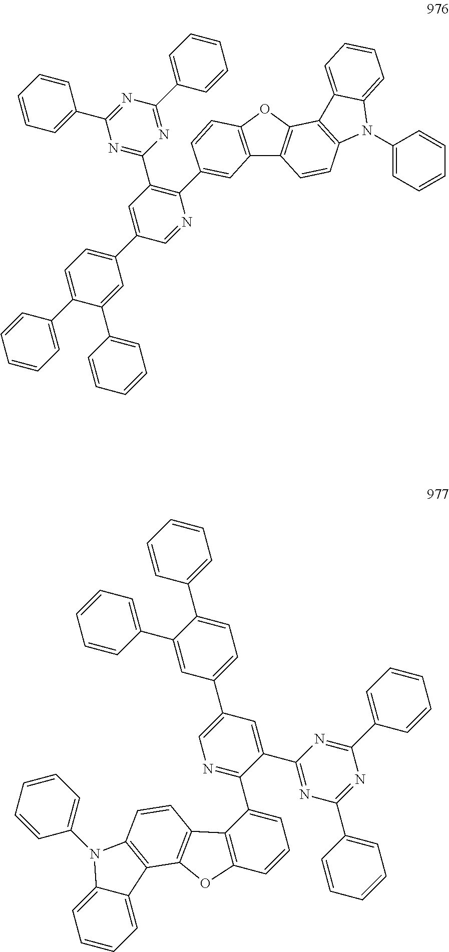 [SMILES: c1ccc(-c2nc(-c3ccccc3)nc(-c3cc(-c4ccc(-c5ccccc5)c(-c5ccccc5)c4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c5c5ccccc5n6-c5ccccc5)c34)n2)cc1]

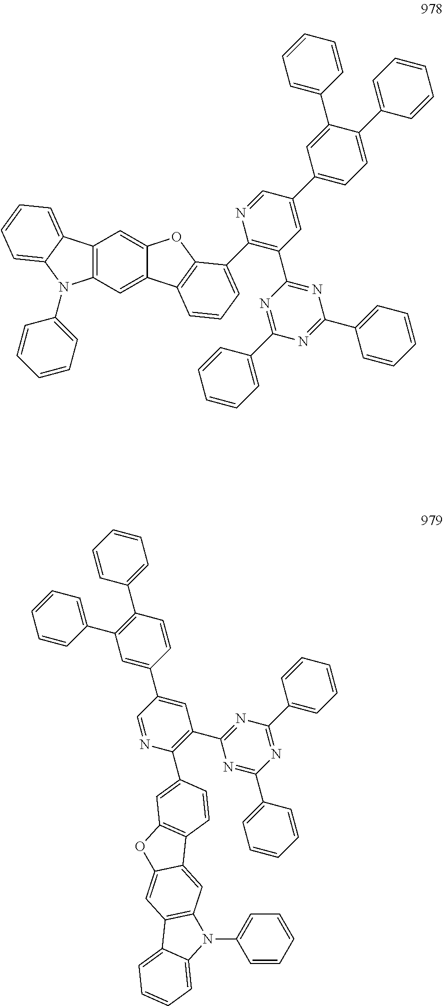 [SMILES: c1ccc(-c2nc(-c3ccccc3)nc(-c3cc(-c4ccc(-c5ccccc5)c(-c5ccccc5)c4)cnc3-c3cccc4c3oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3cc5c6ccccc6n(-c6ccccc6)c5cc34)n2)cc1]

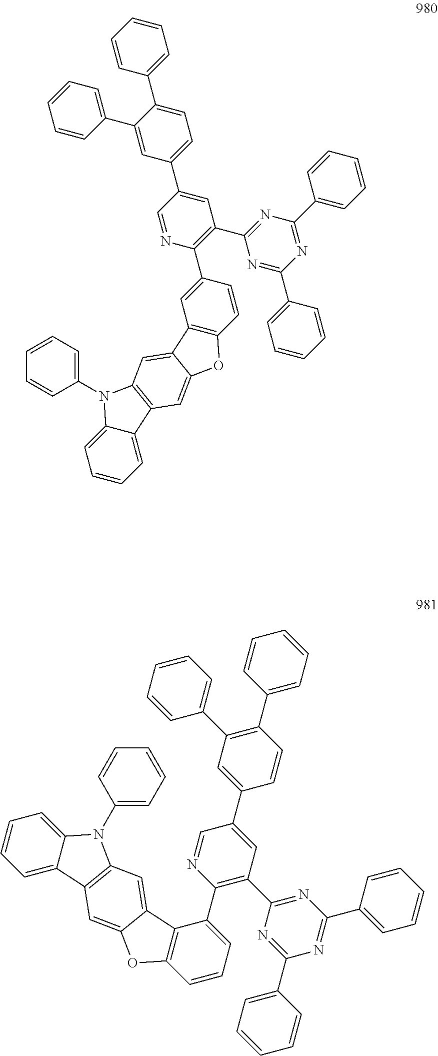 [SMILES: c1ccc(-c2nc(-c3ccccc3)nc(-c3cc(-c4ccc(-c5ccccc5)c(-c5ccccc5)c4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)cnc3-c3cccc4oc5cc6c7ccccc7n(-c7ccccc7)c6cc5c34)n2)cc1]

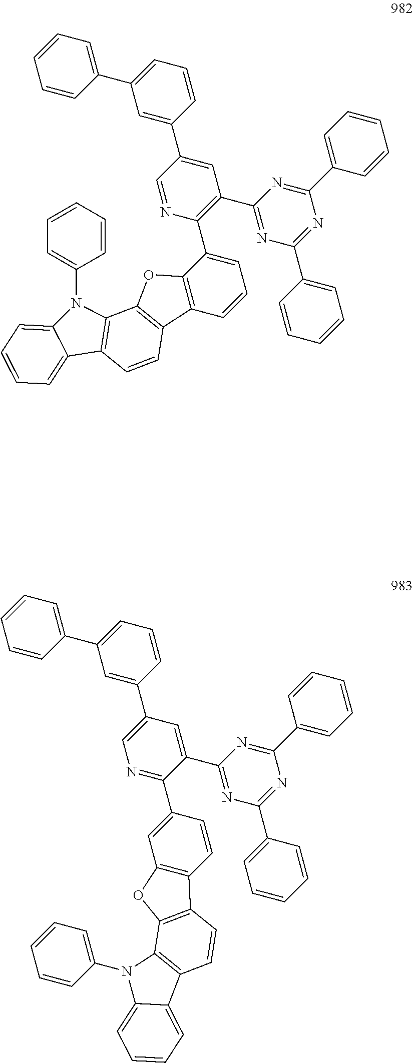 [SMILES: c1ccc(-c2cccc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1]

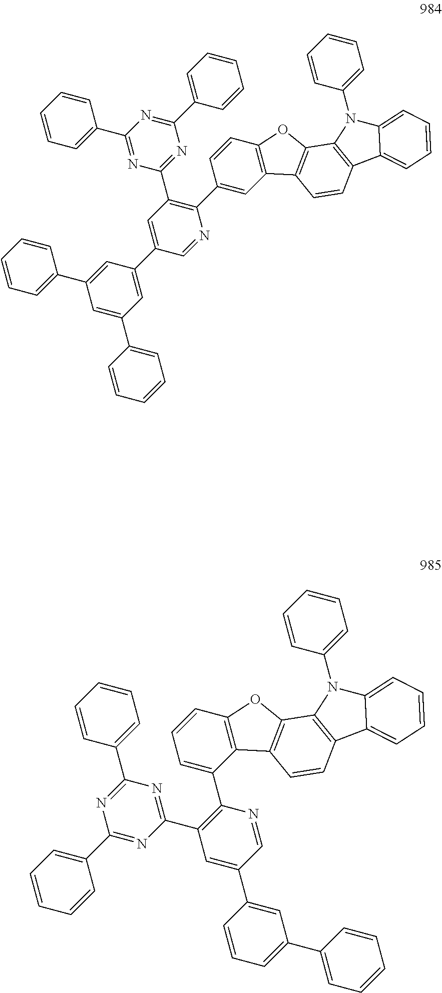 [SMILES: c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

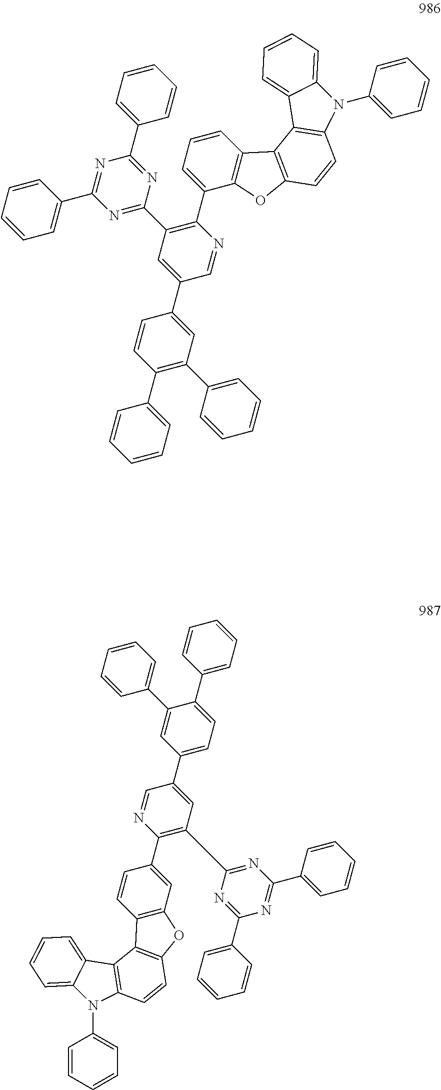 [SMILES: c1ccc(-c2nc(-c3ccccc3)nc(-c3cc(-c4ccc(-c5ccccc5)c(-c5ccccc5)c4)cnc3-c3cccc4c3oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3ccc5c(c6ccccc6n5-c5ccccc5)c34)n2)cc1]

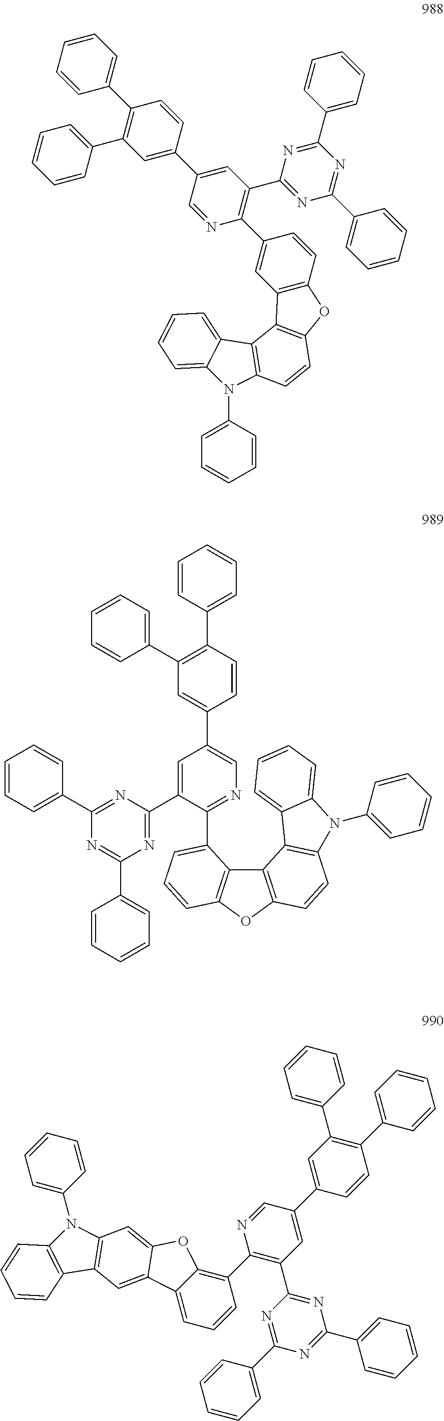 [SMILES: c1ccc(-c2nc(-c3ccccc3)nc(-c3cc(-c4ccc(-c5ccccc5)c(-c5ccccc5)c4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)cnc3-c3cccc4oc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3oc3cc5c(cc34)c3ccccc3n5-c3ccccc3)n2)cc1]

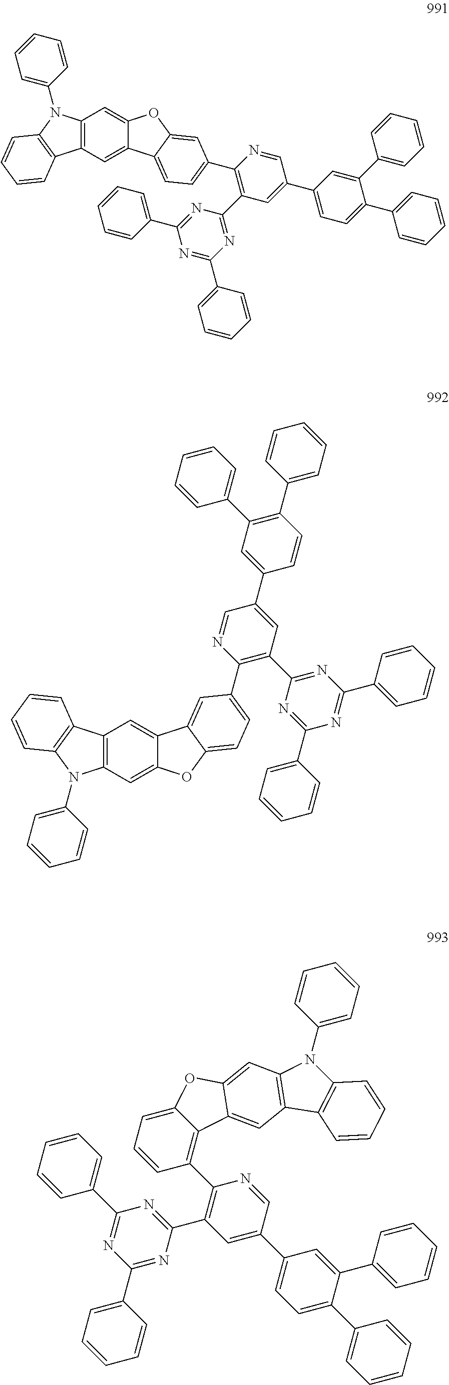 [SMILES: c1ccc(-c2nc(-c3ccccc3)nc(-c3cc(-c4ccc(-c5ccccc5)c(-c5ccccc5)c4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4oc5cc6c(cc5c34)c3ccccc3n6-c3ccccc3)n2)cc1]

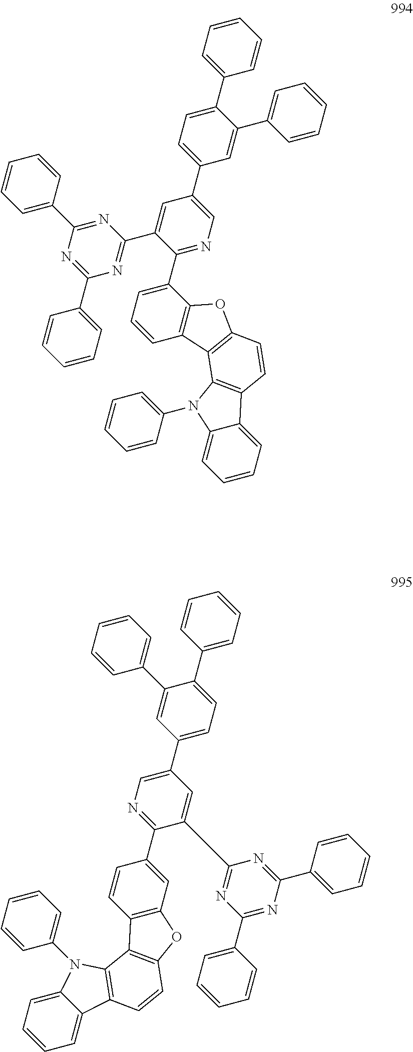 [SMILES: c1ccc(-c2nc(-c3ccccc3)nc(-c3cc(-c4ccc(-c5ccccc5)c(-c5ccccc5)c4)cnc3-c3cccc4c3oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3ccc5c6ccccc6n(-c6ccccc6)c5c34)n2)cc1]

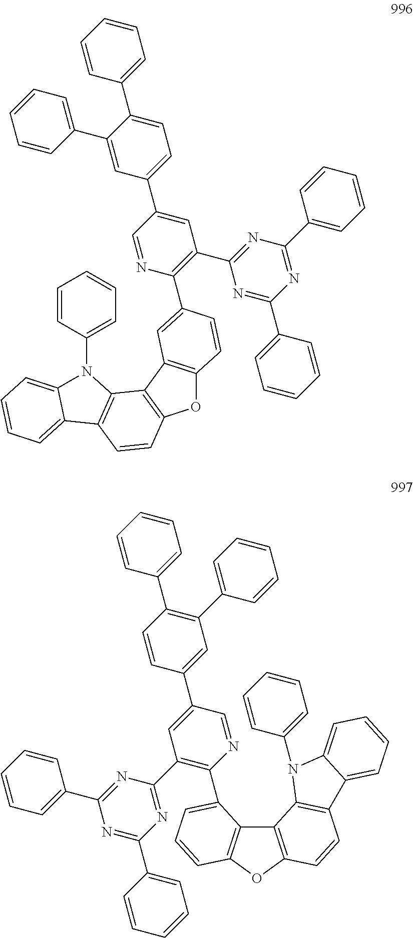 [SMILES: c1ccc(-c2nc(-c3ccccc3)nc(-c3cc(-c4ccc(-c5ccccc5)c(-c5ccccc5)c4)cnc3-c3ccc4oc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)cnc3-c3cccc4oc5ccc6c7ccccc7n(-c7ccccc7)c6c5c34)n2)cc1]

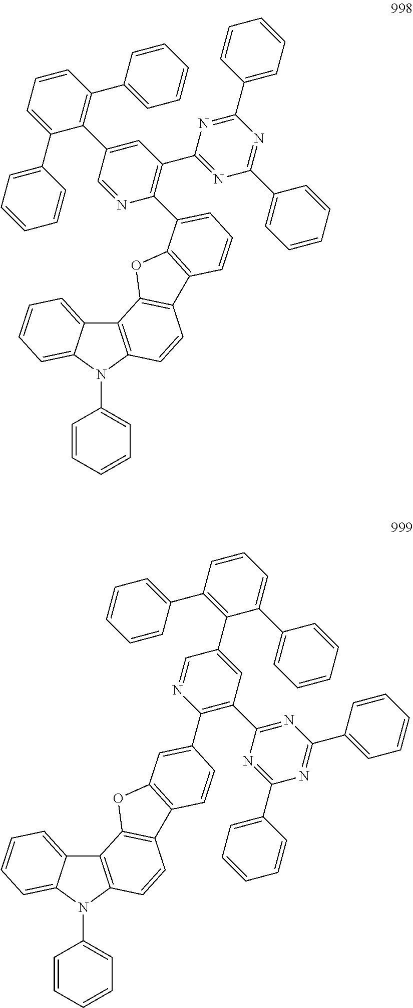 [SMILES: c1ccc(-c2nc(-c3ccccc3)nc(-c3cc(-c4c(-c5ccccc5)cccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)oc3c4ccc4c3c3ccccc3n4-c3ccccc3)n2)cc1]

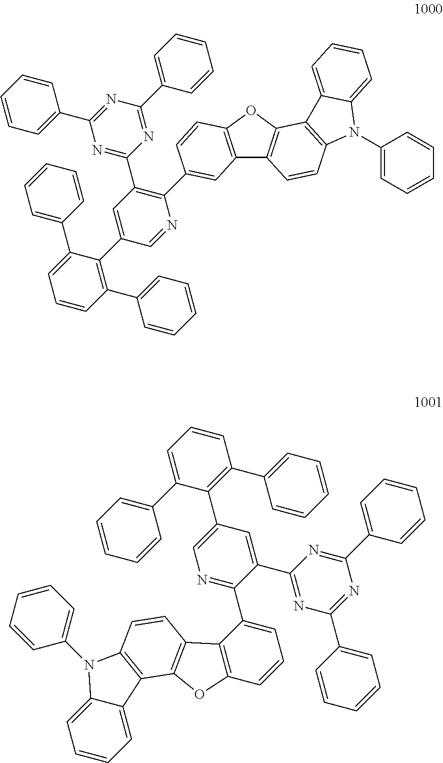 [SMILES: c1ccc(-c2nc(-c3ccccc3)nc(-c3cc(-c4c(-c5ccccc5)cccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4oc5c(ccc6c5c5ccccc5n6-c5ccccc5)c34)n2)cc1]

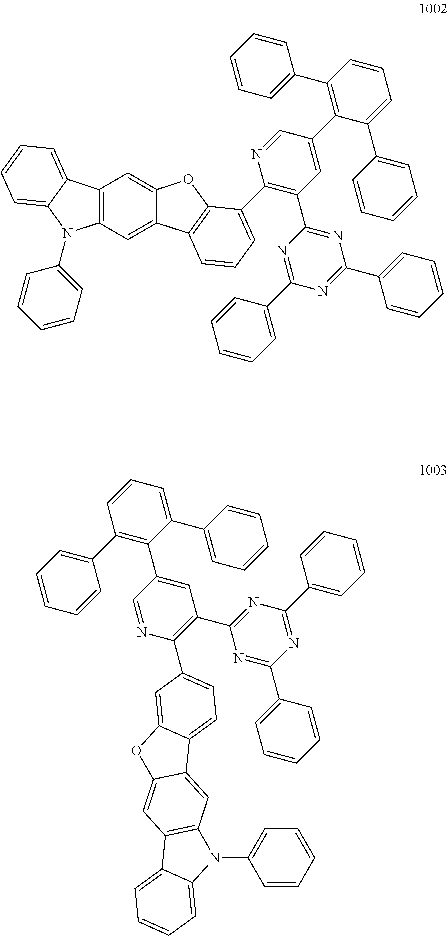 [SMILES: c1ccc(-c2nc(-c3ccccc3)nc(-c3cc(-c4c(-c5ccccc5)cccc4-c4ccccc4)cnc3-c3cccc4c3oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4c(c3)oc3cc5c6ccccc6n(-c6ccccc6)c5cc34)n2)cc1]

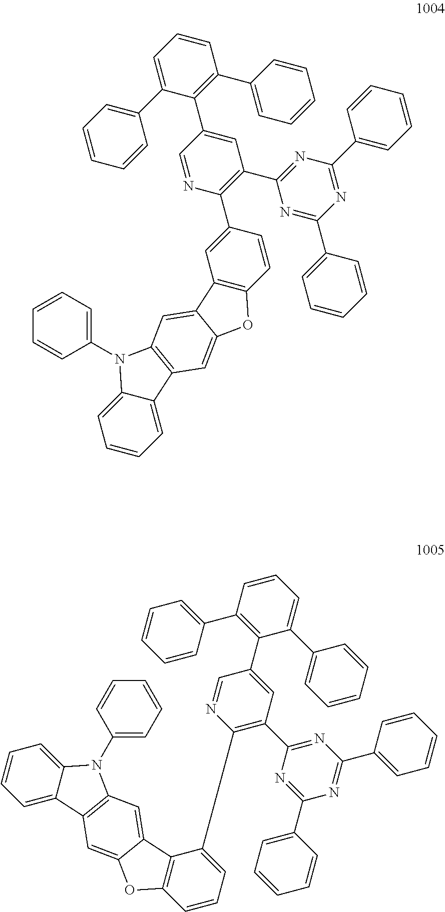 [SMILES: c1ccc(-c2nc(-c3ccccc3)nc(-c3cc(-c4c(-c5ccccc5)cccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4oc5cc6c7ccccc7n(-c7ccccc7)c6cc5c34)n2)cc1]

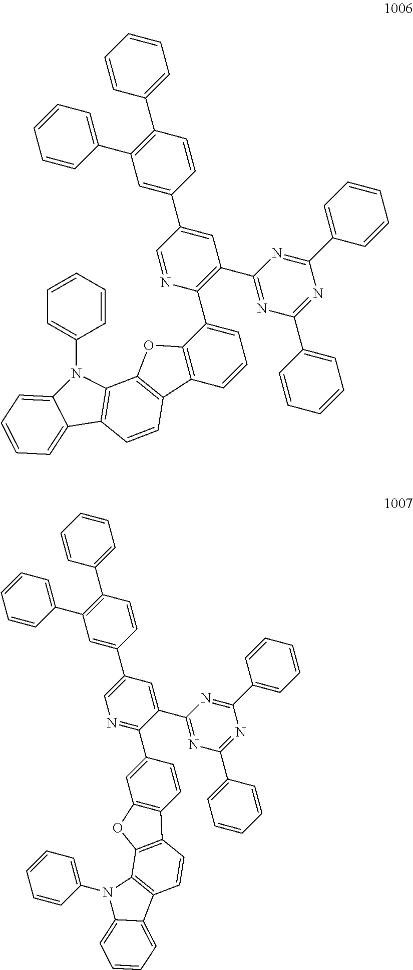 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)oc3c4ccc4c5ccccc5n(-c5ccccc5)c43)n2)cc1]

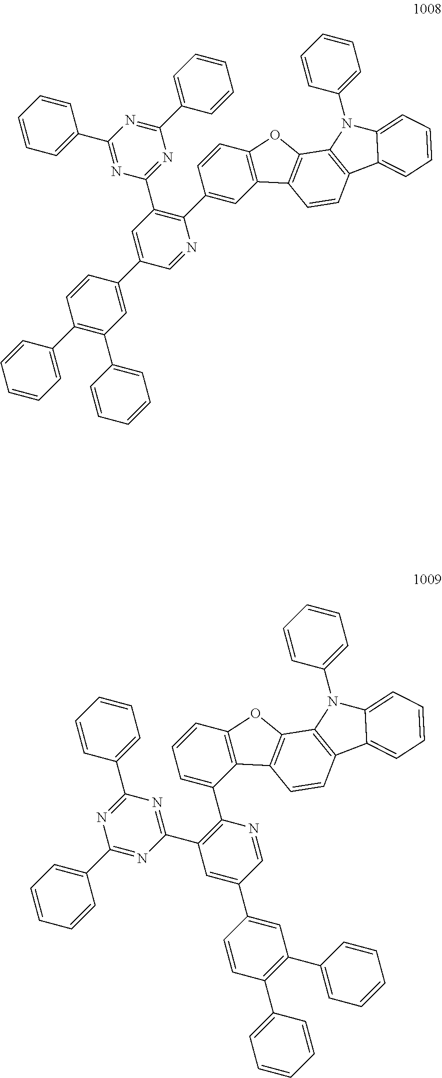 [SMILES: c1ccc(-c2nc(-c3ccccc3)nc(-c3cc(-c4ccc(-c5ccccc5)c(-c5ccccc5)c4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c7ccccc7n(-c7ccccc7)c65)c34)n2)cc1]

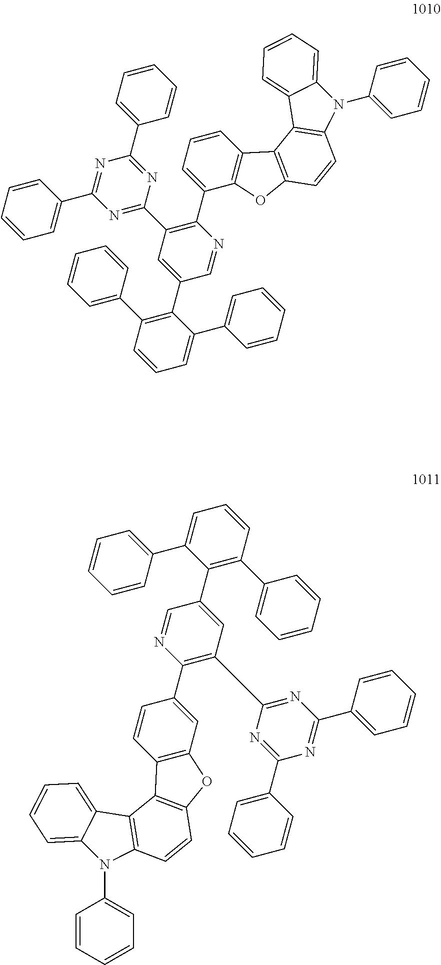 [SMILES: c1ccc(-c2nc(-c3ccccc3)nc(-c3cc(-c4c(-c5ccccc5)cccc4-c4ccccc4)cnc3-c3cccc4c3oc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)cnc3-c3ccc4c(c3)oc3ccc5c(c6ccccc6n5-c5ccccc5)c34)n2)cc1]

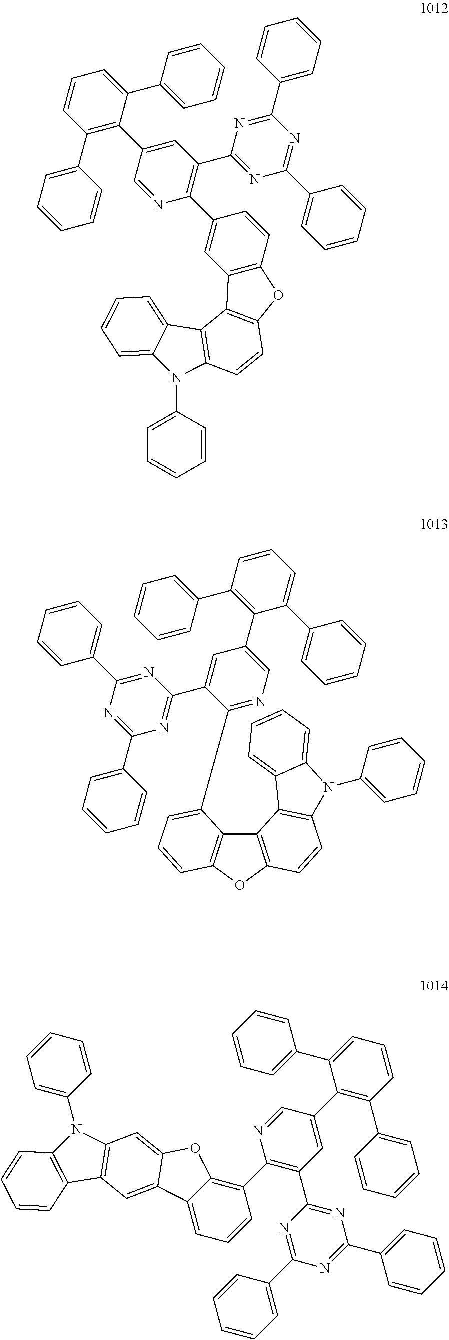 [SMILES: c1ccc(-c2nc(-c3ccccc3)nc(-c3cc(-c4c(-c5ccccc5)cccc4-c4ccccc4)cnc3-c3ccc4oc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)cnc3-c3cccc4oc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)cnc3-c3cccc4c3oc3cc5c(cc34)c3ccccc3n5-c3ccccc3)n2)cc1]

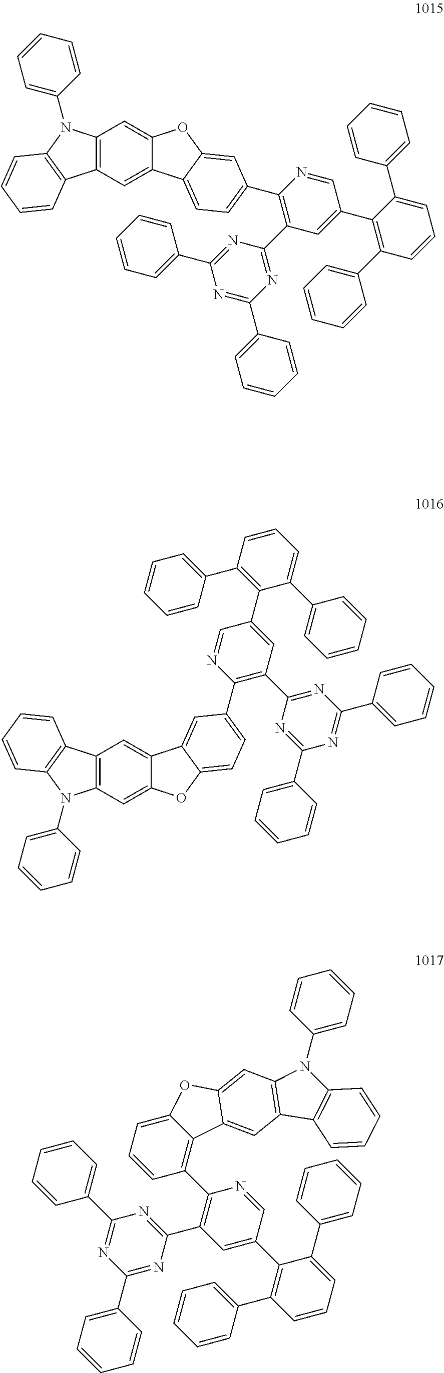 [SMILES: c1ccc(-c2nc(-c3ccccc3)nc(-c3cc(-c4c(-c5ccccc5)cccc4-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4oc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4oc5cc6c(cc5c34)c3ccccc3n6-c3ccccc3)n2)cc1]

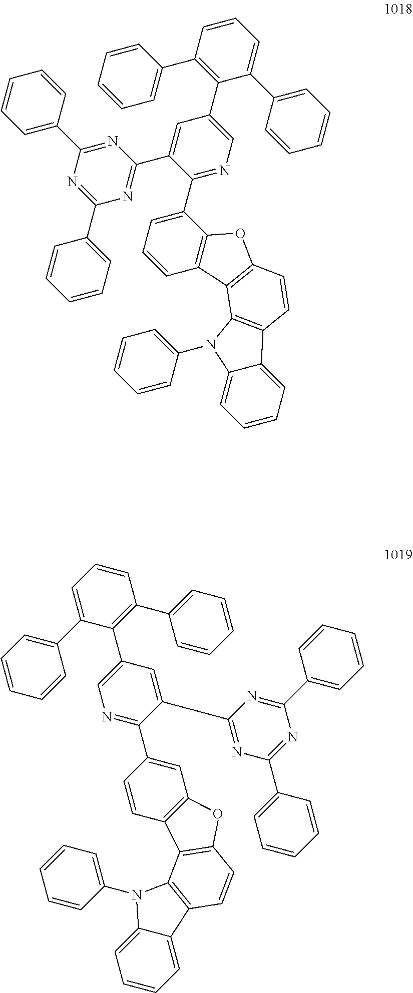 [SMILES: c1ccc(-c2nc(-c3ccccc3)nc(-c3cc(-c4c(-c5ccccc5)cccc4-c4ccccc4)cnc3-c3cccc4c3oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)oc3ccc5c6ccccc6n(-c6ccccc6)c5c34)n2)cc1]

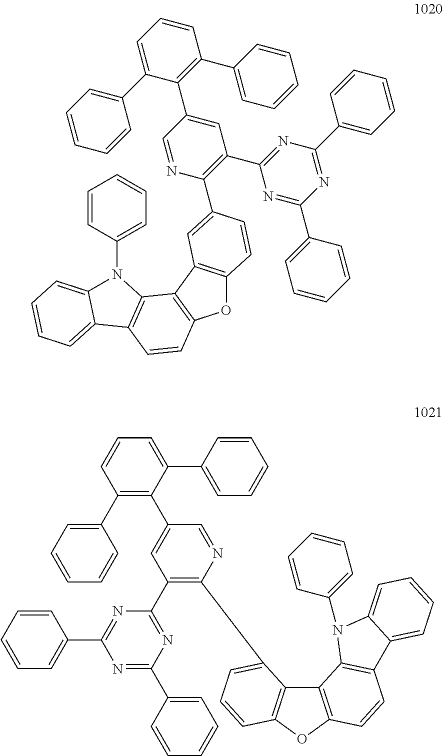 [SMILES: c1ccc(-c2nc(-c3ccccc3)nc(-c3cc(-c4c(-c5ccccc5)cccc4-c4ccccc4)cnc3-c3ccc4oc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)cnc3-c3cccc4oc5ccc6c7ccccc7n(-c7ccccc7)c6c5c34)n2)cc1]

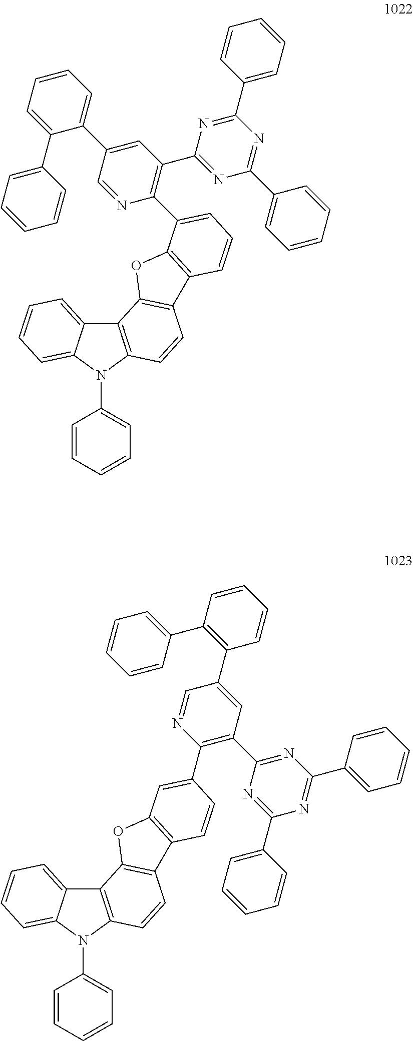 [SMILES: c1ccc(-c2nc(-c3ccccc3)nc(-c3cc(-c4ccccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)oc3c4ccc4c3c3ccccc3n4-c3ccccc3)n2)cc1]

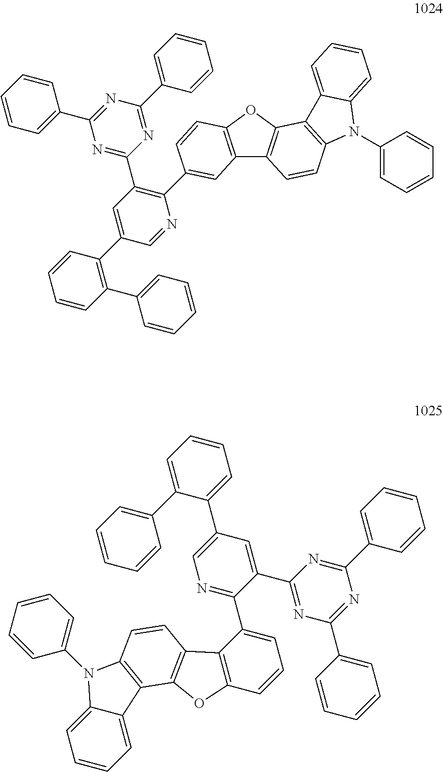 [SMILES: c1ccc(-c2nc(-c3ccccc3)nc(-c3cc(-c4ccccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4oc5c(ccc6c5c5ccccc5n6-c5ccccc5)c34)n2)cc1]

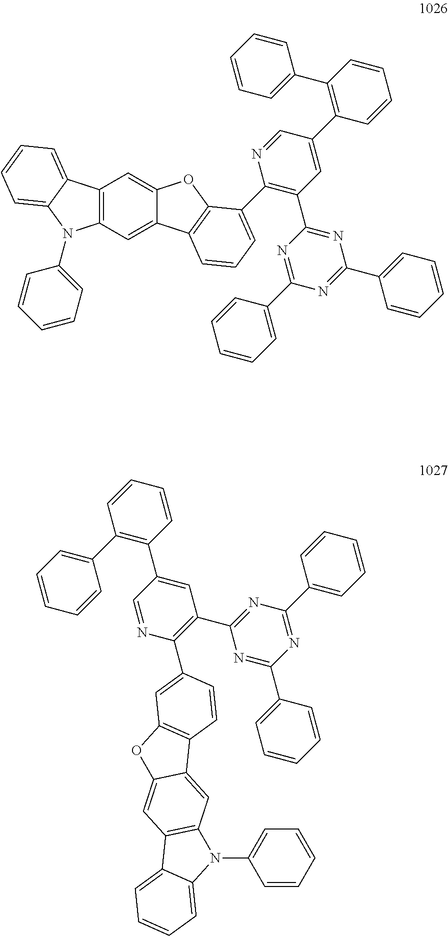 [SMILES: c1ccc(-c2nc(-c3ccccc3)nc(-c3cc(-c4ccccc4-c4ccccc4)cnc3-c3cccc4c3oc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4c(c3)oc3cc5c6ccccc6n(-c6ccccc6)c5cc34)n2)cc1]

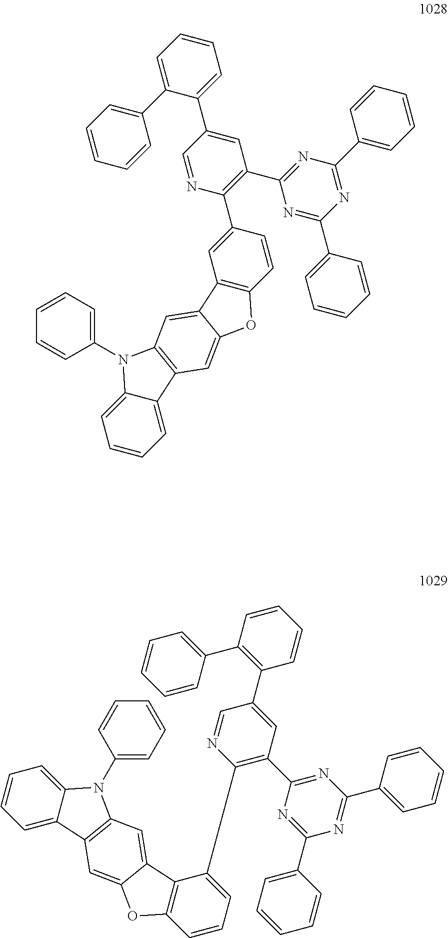 [SMILES: c1ccc(-c2nc(-c3ccccc3)nc(-c3cc(-c4ccccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4oc5cc6c7ccccc7n(-c7ccccc7)c6cc5c34)n2)cc1]

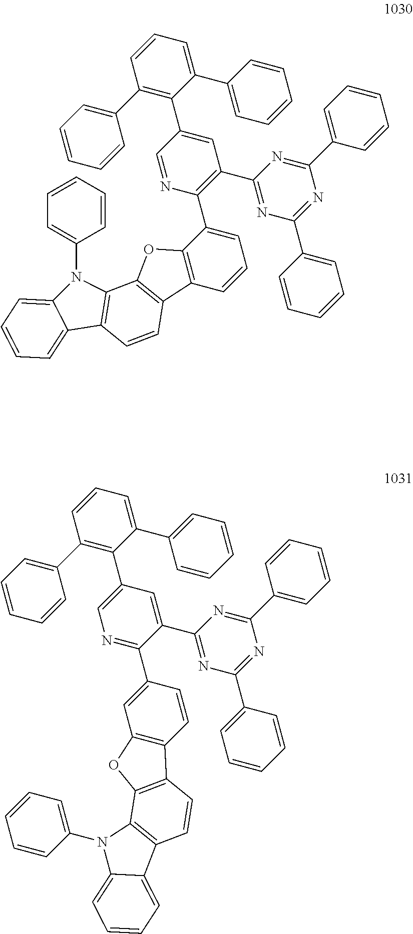 [SMILES: c1ccc(-c2nc(-c3ccccc3)nc(-c3cc(-c4c(-c5ccccc5)cccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4c(c3)oc3c4ccc4c5ccccc5n(-c5ccccc5)c43)n2)cc1]

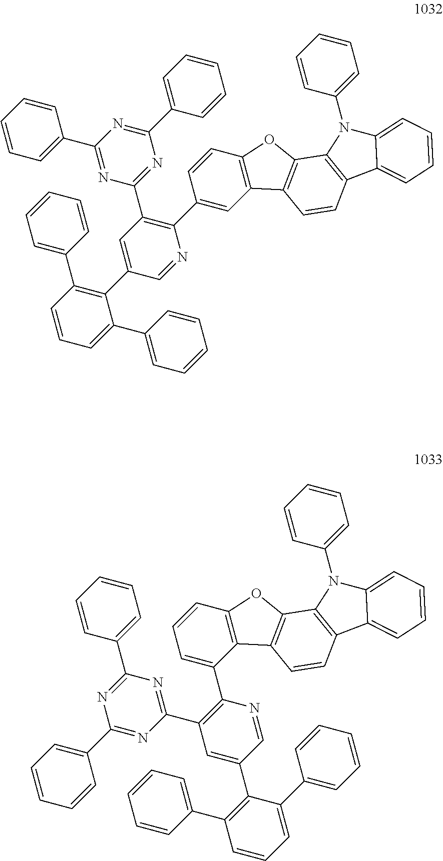 [SMILES: c1ccc(-c2nc(-c3ccccc3)nc(-c3cc(-c4c(-c5ccccc5)cccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4c(-c5ccccc5)cccc4-c4ccccc4)cnc3-c3cccc4oc5c(ccc6c7ccccc7n(-c7ccccc7)c65)c34)n2)cc1]

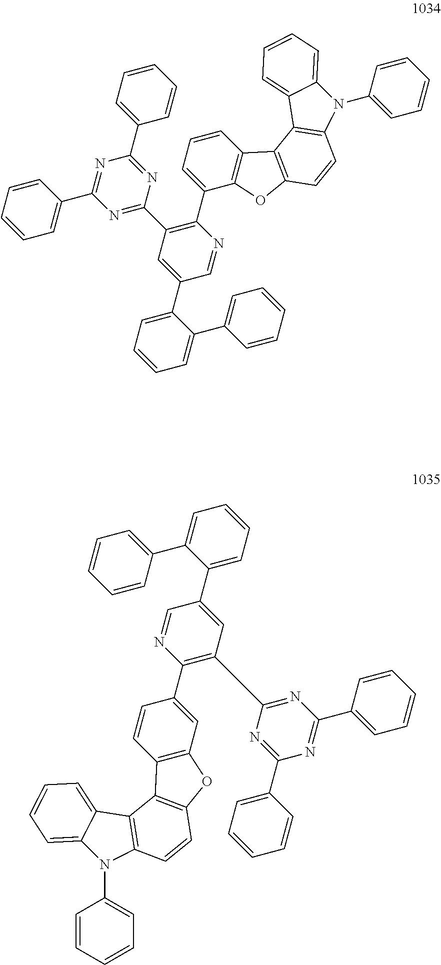 [SMILES: c1ccc(-c2nc(-c3ccccc3)nc(-c3cc(-c4ccccc4-c4ccccc4)cnc3-c3cccc4c3oc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)oc3ccc5c(c6ccccc6n5-c5ccccc5)c34)n2)cc1]

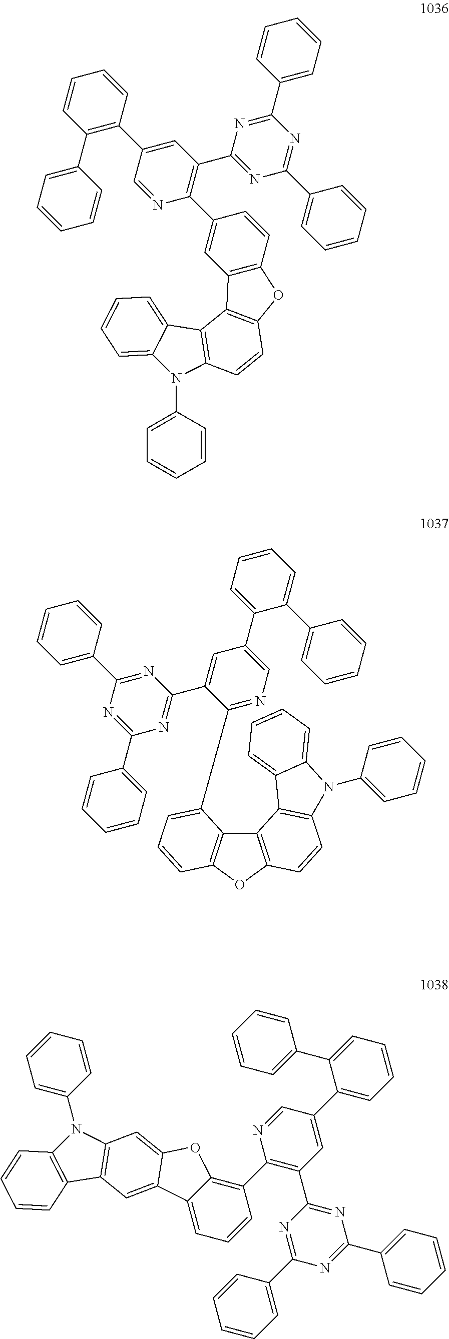 [SMILES: c1ccc(-c2nc(-c3ccccc3)nc(-c3cc(-c4ccccc4-c4ccccc4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4oc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3oc3cc5c(cc34)c3ccccc3n5-c3ccccc3)n2)cc1]

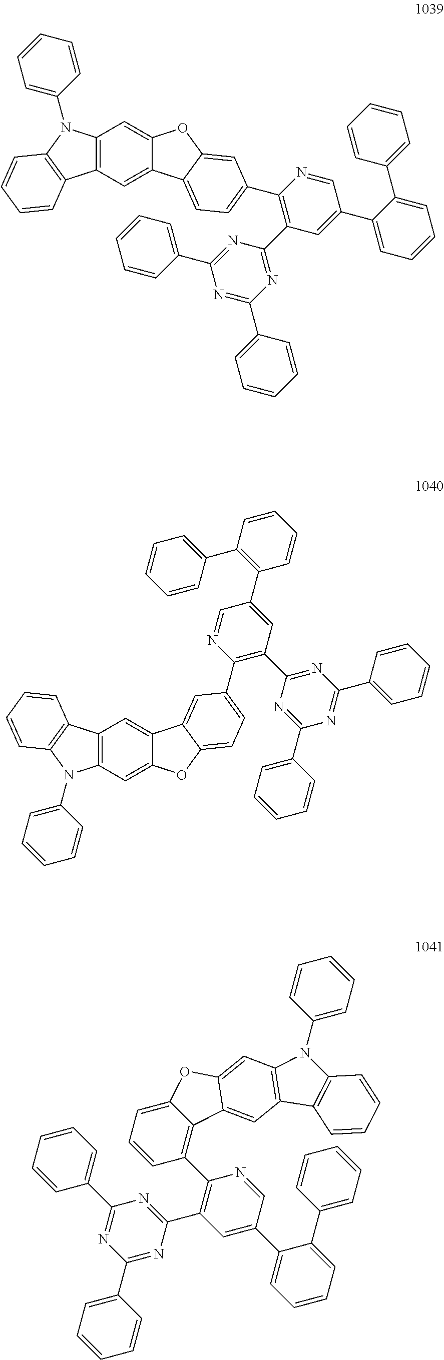 [SMILES: c1ccc(-c2nc(-c3ccccc3)nc(-c3cc(-c4ccccc4-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4oc5cc6c(cc5c34)c3ccccc3n6-c3ccccc3)n2)cc1]

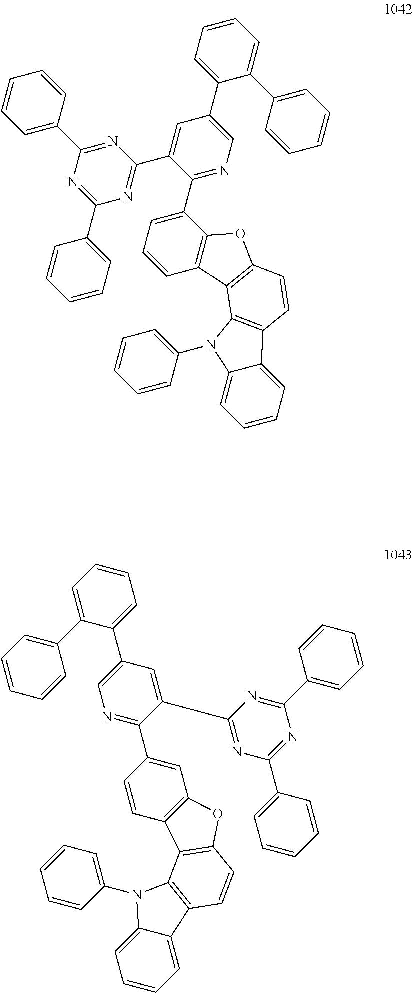 [SMILES: c1ccc(-c2nc(-c3ccccc3)nc(-c3cc(-c4ccccc4-c4ccccc4)cnc3-c3cccc4c3oc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4c(c3)oc3ccc5c6ccccc6n(-c6ccccc6)c5c34)n2)cc1]

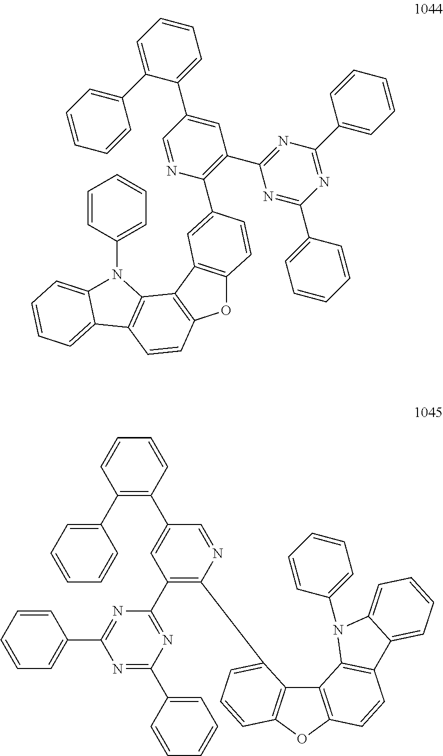 [SMILES: c1ccc(-c2nc(-c3ccccc3)nc(-c3cc(-c4ccccc4-c4ccccc4)cnc3-c3ccc4oc5ccc6c7ccccc7n(-c7ccccc7)c6c5c4c3)n2)cc1 ; c1ccc(-c2nc(-c3ccccc3)nc(-c3cc(-c4ccccc4-c4ccccc4)cnc3-c3cccc4oc5ccc6c7ccccc7n(-c7ccccc7)c6c5c34)n2)cc1]

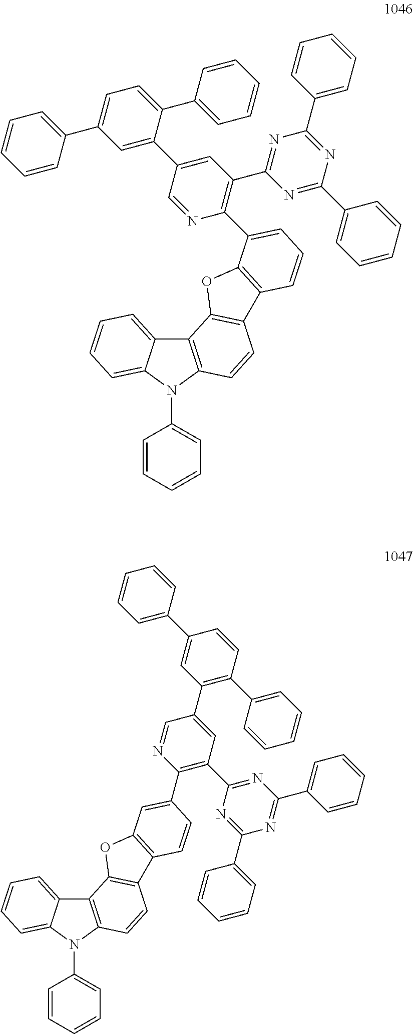 [SMILES: c1ccc(-c2ccc(-c3ccccc3)c(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1]

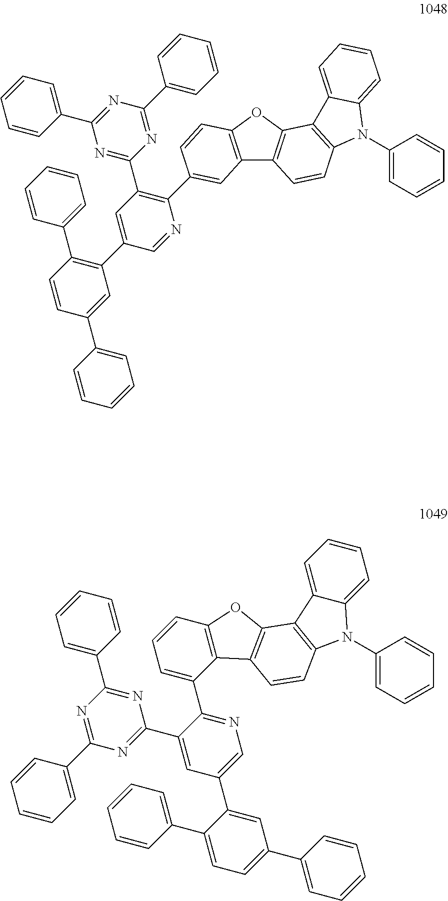 [SMILES: c1ccc(-c2ccc(-c3ccccc3)c(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

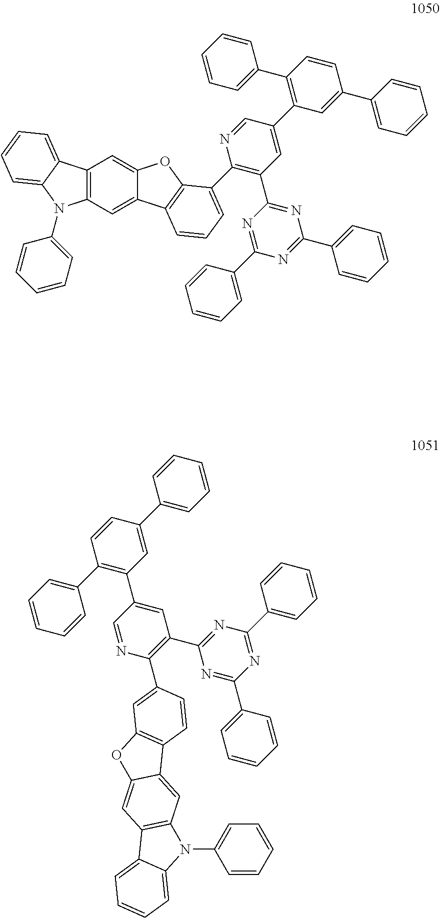 [SMILES: c1ccc(-c2ccc(-c3ccccc3)c(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1]

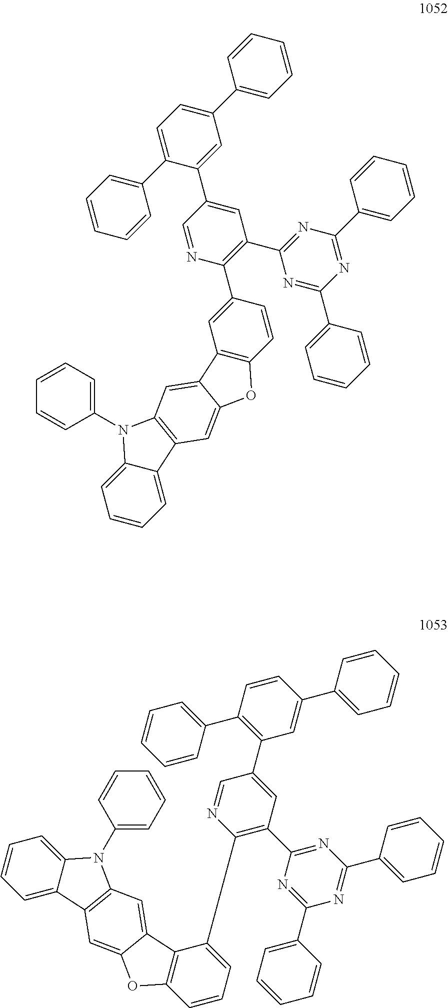 [SMILES: c1ccc(-c2ccc(-c3ccccc3)c(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

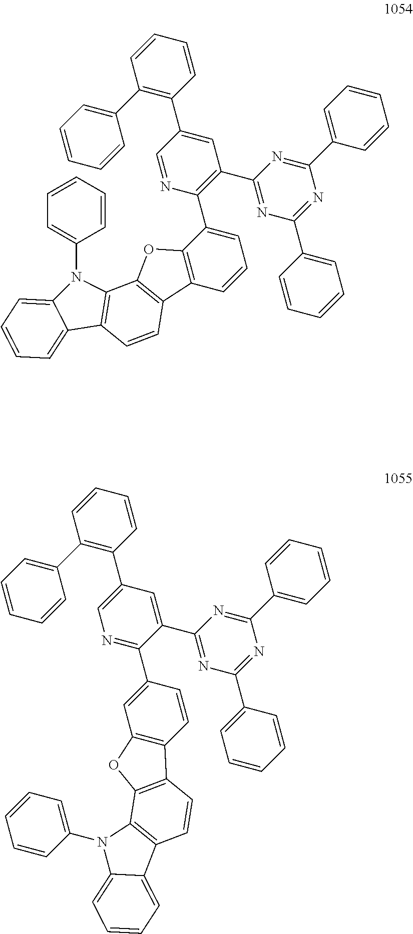 [SMILES: c1ccc(-c2nc(-c3ccccc3)nc(-c3cc(-c4ccccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)oc3c4ccc4c5ccccc5n(-c5ccccc5)c43)n2)cc1]

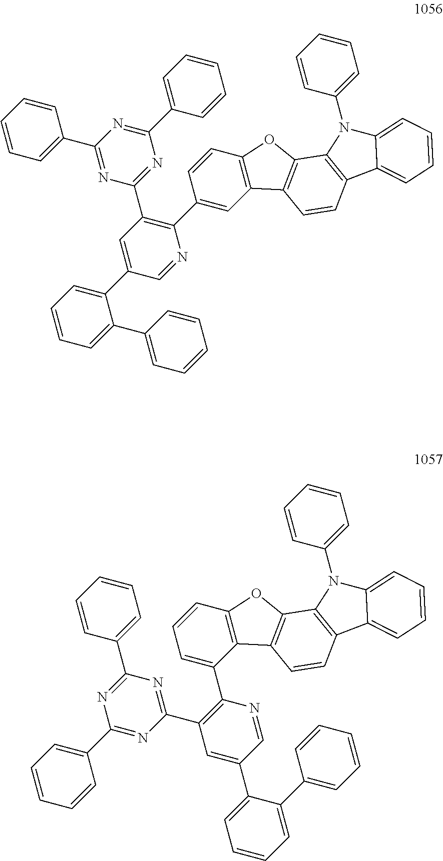 [SMILES: c1ccc(-c2nc(-c3ccccc3)nc(-c3cc(-c4ccccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4oc5c(ccc6c7ccccc7n(-c7ccccc7)c65)c34)n2)cc1]

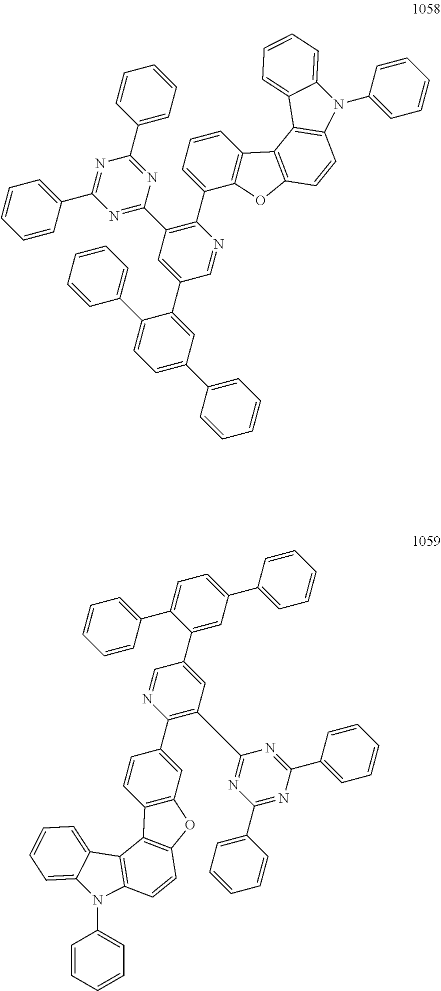 [SMILES: c1ccc(-c2ccc(-c3ccccc3)c(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1]

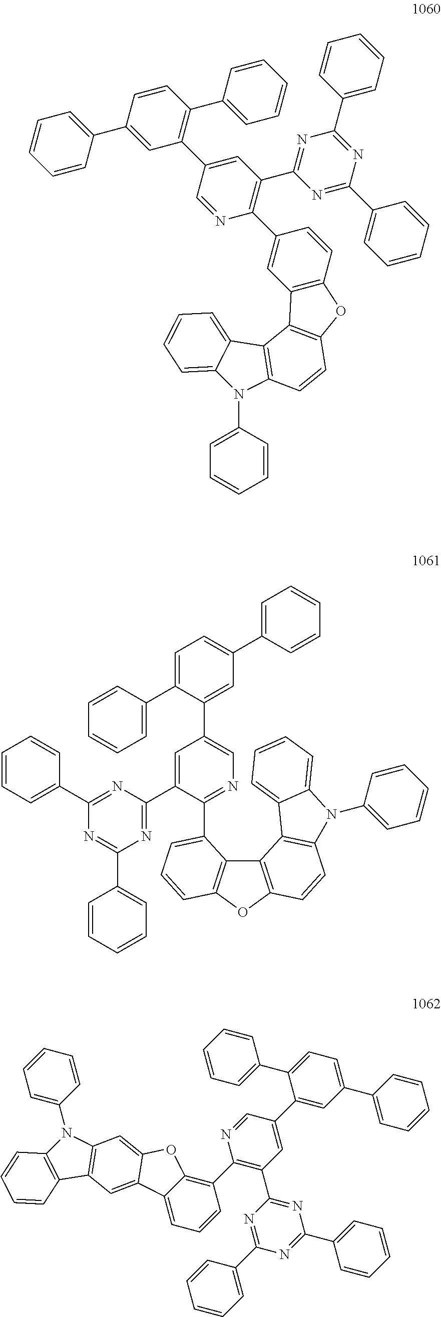 [SMILES: c1ccc(-c2ccc(-c3ccccc3)c(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4cccc5c4oc4cc6c(cc45)c4ccccc4n6-c4ccccc4)c(-c4nc(-c5ccccc5)nc(-c5ccccc5)n4)c3)c2)cc1]

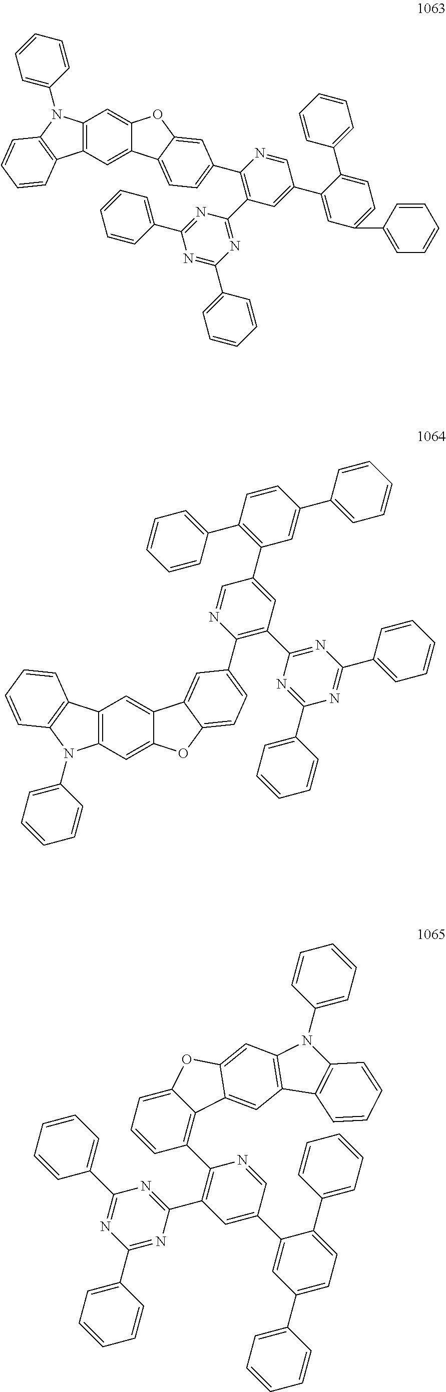 [SMILES: c1ccc(-c2ccc(-c3ccccc3)c(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c(cc6c45)c4ccccc4n7-c4ccccc4)c(-c4nc(-c5ccccc5)nc(-c5ccccc5)n4)c3)c2)cc1]

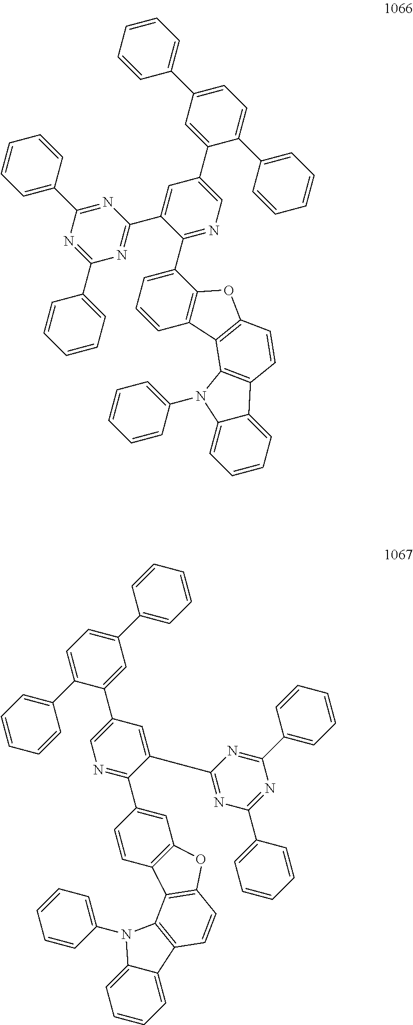 [SMILES: c1ccc(-c2ccc(-c3ccccc3)c(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1]

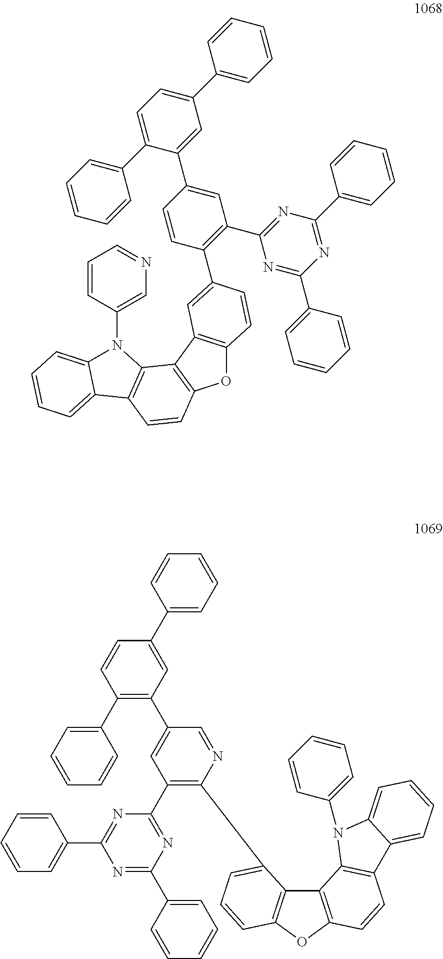 [SMILES: c1ccc(-c2ccc(-c3ccccc3)c(-c3ccc(-c4ccc5oc6ccc7c8ccccc8n(-c8cccnc8)c7c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

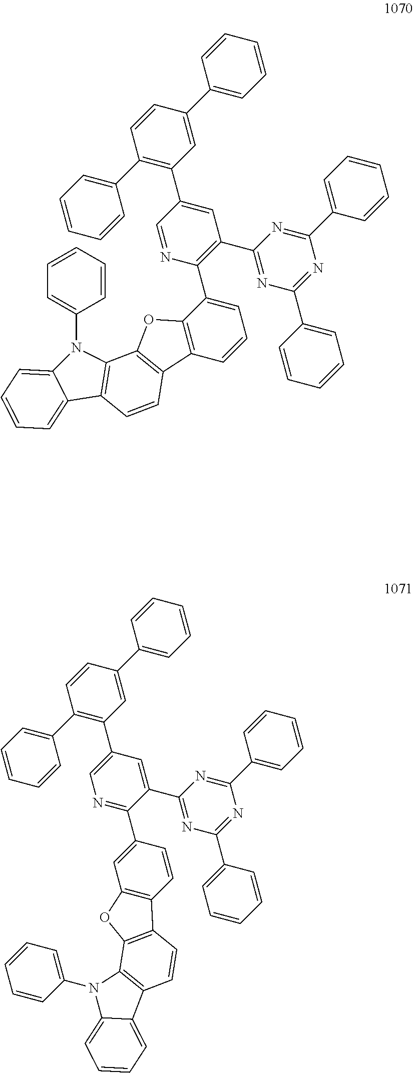 [SMILES: c1ccc(-c2ccc(-c3ccccc3)c(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1]

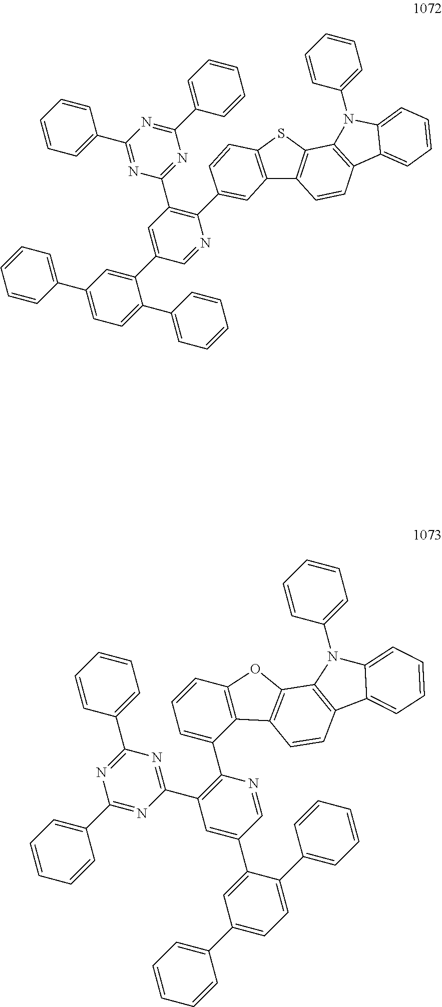 [SMILES: c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

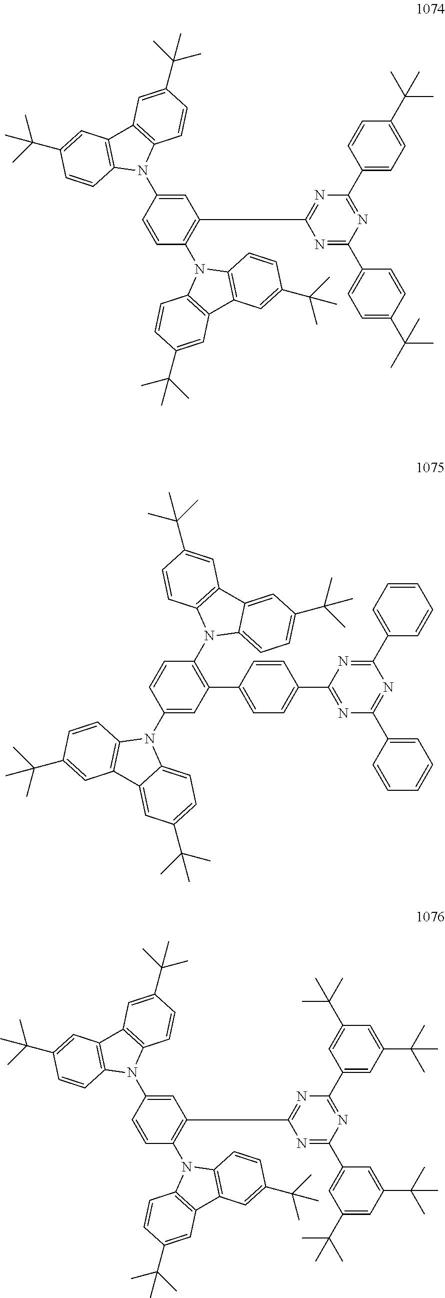

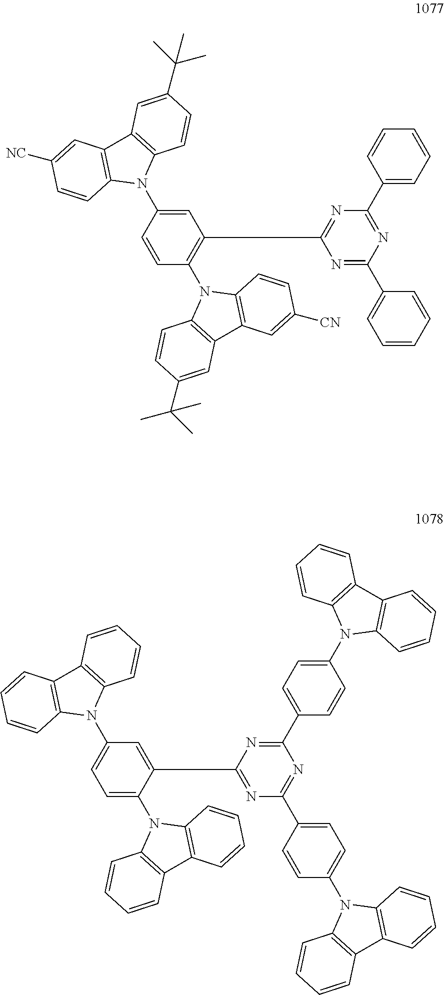 [SMILES: CC(C)(C)c1ccc2c(c1)c1cc(C#N)ccc1n2-c1ccc(-n2c3ccc(C#N)cc3c3cc(C(C)(C)C)ccc32)c(-c2nc(-c3ccccc3)nc(-c3ccccc3)n2)c1 ; c1ccc2c(c1)c1ccccc1n2-c1ccc(-c2nc(-c3ccc(-n4c5ccccc5c5ccccc54)cc3)nc(-c3cc(-n4c5ccccc5c5ccccc54)ccc3-n3c4ccccc4c4ccccc43)n2)cc1]

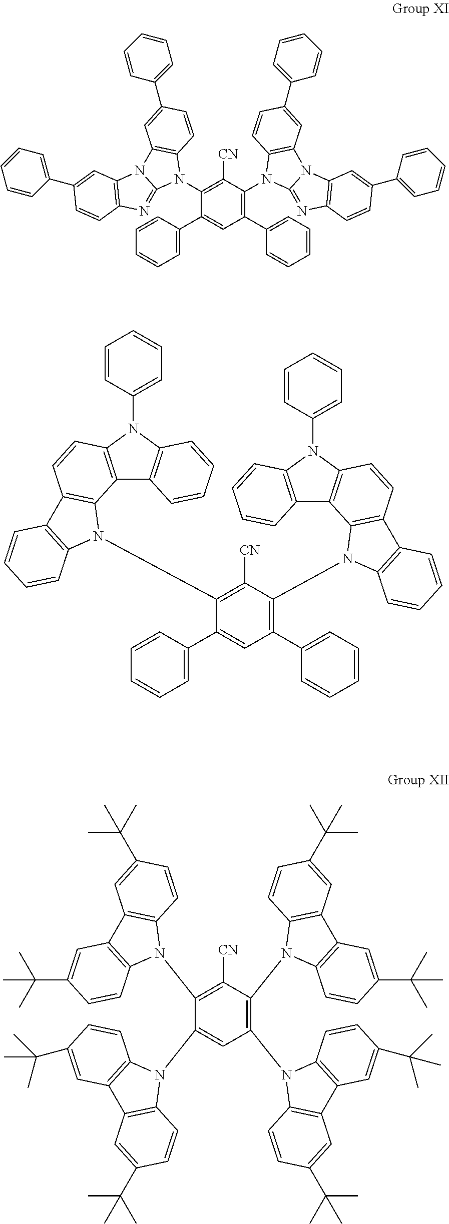 [SMILES: N#Cc1c(-n2c3ccc(-c4ccccc4)cc3n3c4cc(-c5ccccc5)ccc4nc23)c(-c2ccccc2)cc(-c2ccccc2)c1-n1c2ccc(-c3ccccc3)cc2n2c3cc(-c4ccccc4)ccc3nc12 ; N#Cc1c(-n2c3ccccc3c3ccc4c(c5ccccc5n4-c4ccccc4)c32)c(-c2ccccc2)cc(-c2ccccc2)c1-n1c2ccccc2c2ccc3c(c4ccccc4n3-c3ccccc3)c21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-n2c3ccc(C(C)(C)C)cc3c3cc(C(C)(C)C)ccc32)c(C#N)c1-n1c2ccc(C(C)(C)C)cc2c2cc(C(C)(C)C)ccc21]

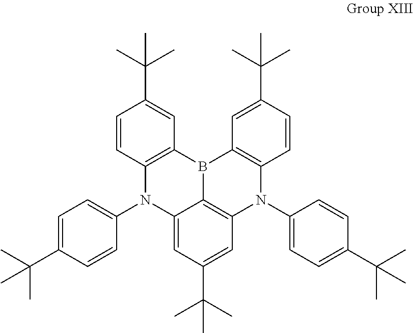 [SMILES: CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1]

### Hole Transport Region 12

The hole transport region 12 may be located between the first electrode 11 and the emission layer 15 of the organic light-emitting device 10.

The hole transport region 12 may have a single-layered structure or a multi-layered structure.

For example, the hole transport region 12 may have a hole injection layer, a hole transport layer, a hole injection layer/hole transport layer structure, a hole injection layer/first hole transport layer/second hole transport layer structure, a hole transport layer/middle layer structure, a hole injection layer/hole transport layer/middle layer structure, a hole transport layer/electron-blocking layer structure, or a hole injection layer/hole transport layer/electron-blocking layer structure, but embodiments of the present disclosure are not limited thereto.

The hole transport region 12 may include any compound having hole-transporting properties.

For example, the hole transport region 12 may include an amine-containing compound.

In one or more embodiments, the hole transport region 1 may include at least one of a compound represented by Formula 201 to a compound represented by Formula 205, but embodiments of the present disclosure are not limited thereto:

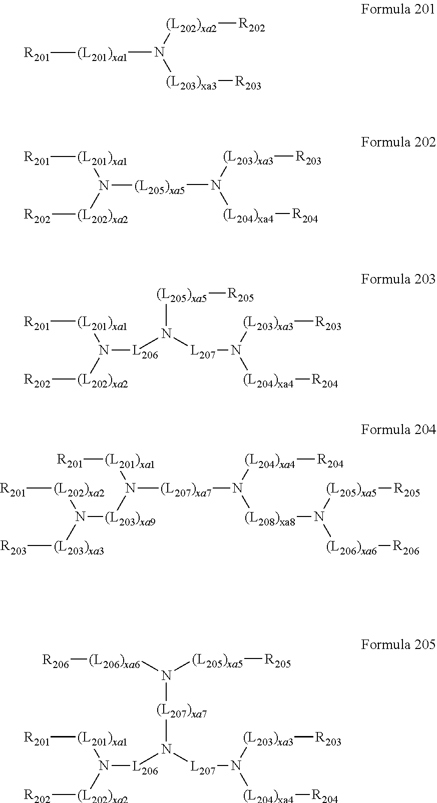

wherein, in Formulae 201 to 205,

L₂₀₁ to L₂₀₉ may each independently be O, S, a substituted or unsubstituted C₅-C₆₀ carbocyclic group, or a substituted or unsubstituted C₁-C₆₀ heterocyclic group,

xa1 to xa9 may each independently be an integer from 0 to 5, and

R₂₀₁ to R₂₀₆ may each independently be a substituted or unsubstituted C₃-C₁₀cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group, wherein neighboring two groups of R₂₀₁ to R₂₀₆ may optionally be linked to each other via a single bond, a dimethyl-methylene group, or a diphenyl-methylene group.

For example, in one or more embodiments,

L₂₀₁ to L₂₀₉ may be a phenyl group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, and a triindolobenzene group, each unsubstituted or substituted with deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a quaterphenyl group, or —Si(Q₁₁)(Q₁₂)(Q₁₃),

xa1 to xa9 may each independently be 0, 1, or 2, and

R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indeno carbazolyl group, an indolocarbazolyl group, a benzofurocarbazolyl group, or a benzothienocarbazolyl group, each unsubstituted or substituted with at least one of deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), or —N(Q₃₁)(Q₃₂),

wherein Q₁₁ to Q₁₃ and Q₃₁ to Q₃₃ may each independently be a C₁-C₁₀ alkyl group, a C₁-C₁₀ alkoxy group, a phenyl group, a biphenyl group, a terphenyl group, or a naphthyl group.

In one or more embodiments, the hole transport region 12 may include a carbazole-containing amine compound.

In one or more embodiments, the hole transport region 12 may include a carbazole-containing amine compound and a carbazole-free amine compound.

The carbazole-containing amine compound may be s, for example, a compound represented by Formula 201 including a carbazole group and further including at least one of a dibenzofuran group, a dibenzothiophene group, a fluorene group, a spiro-bifluorene group, an indenocarbazole group, an indolocarbazole group, a benzofurocarbazole group, or a benzothienocarbazole group.

The carbazole-free amine compound may be, for example, a compound represented by Formula 201 which does not include a carbazole group and which includes at least one of a dibenzofuran group, a dibenzothiophene group, a fluorene group, a spiro-bifluorene group, an indenocarbazole group, an indolocarbazole group, a benzofurocarbazole group, or a benzothienocarbazole group.

In one or more embodiments, the hole transport region 12 may include at least one compound represented by Formulae 201 or 202.

In one or more embodiments, the hole transport region 12 may include at least one compound represented by Formulae 201-1, 202-1, or 201-2, but embodiments of the present disclosure are not limited thereto.

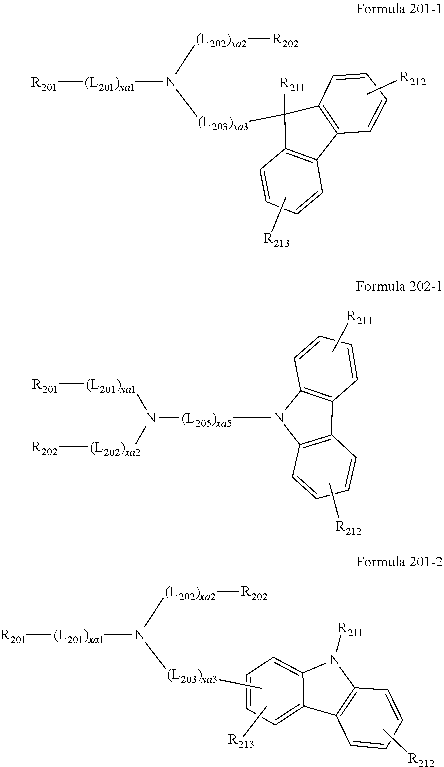

In Formulae 201-1, 202-1, and 201-2, L₂₀₁ to L₂₀₃, L₂₀₅, xa1 to xa3, xa5, R₂₀₁ and R₂₀₂ are the same as described herein, and R₂₁₁ to R₂₁₃ are each independently hydrogen, deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenyla fluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, or a pyridinyl group.

For example, the hole transport region 12 may include at least one of Compounds HT1 to HT39, but embodiments of the present disclosure are not limited thereto.

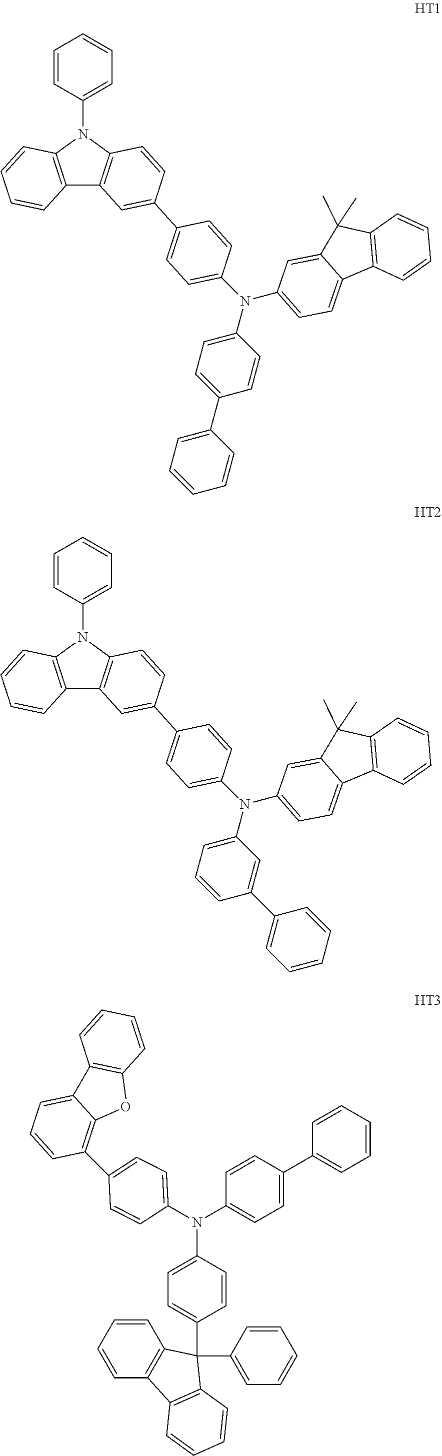 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

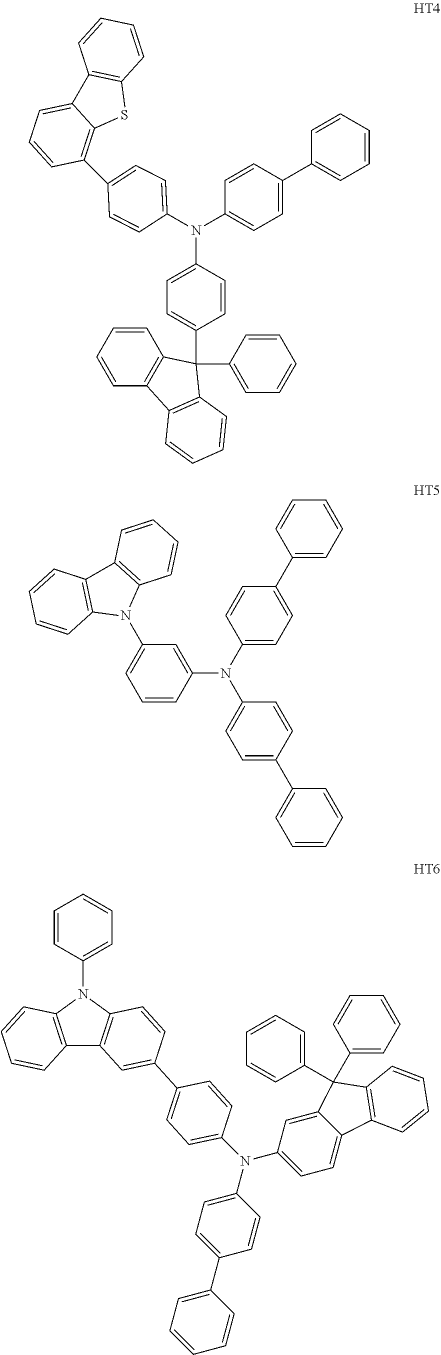 [SMILES: c1ccc(-c2ccc(N(c3ccc(-c4cccc5c4sc4ccccc45)cc3)c3ccc(C4(c5ccccc5)c5ccccc5-c5ccccc54)cc3)cc2)cc1 ; c1ccc(-c2ccc(N(c3ccc(-c4ccccc4)cc3)c3cccc(-n4c5ccccc5c5ccccc54)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

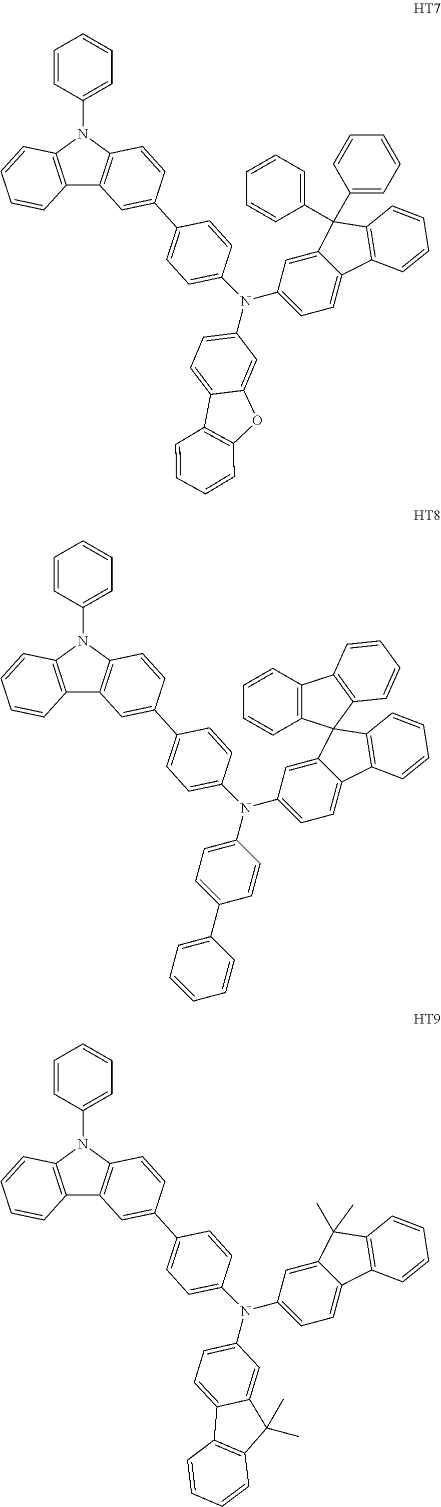 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

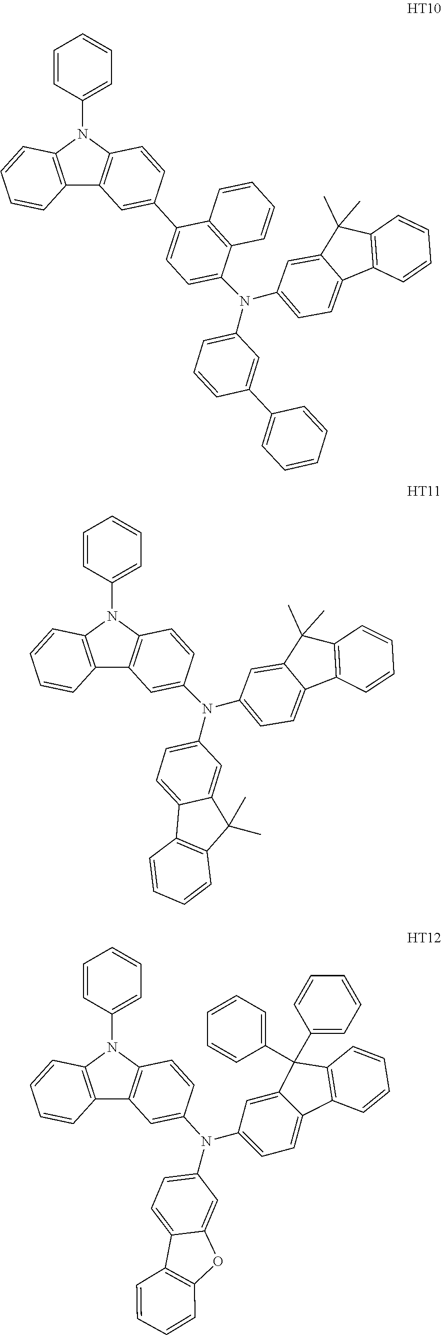 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

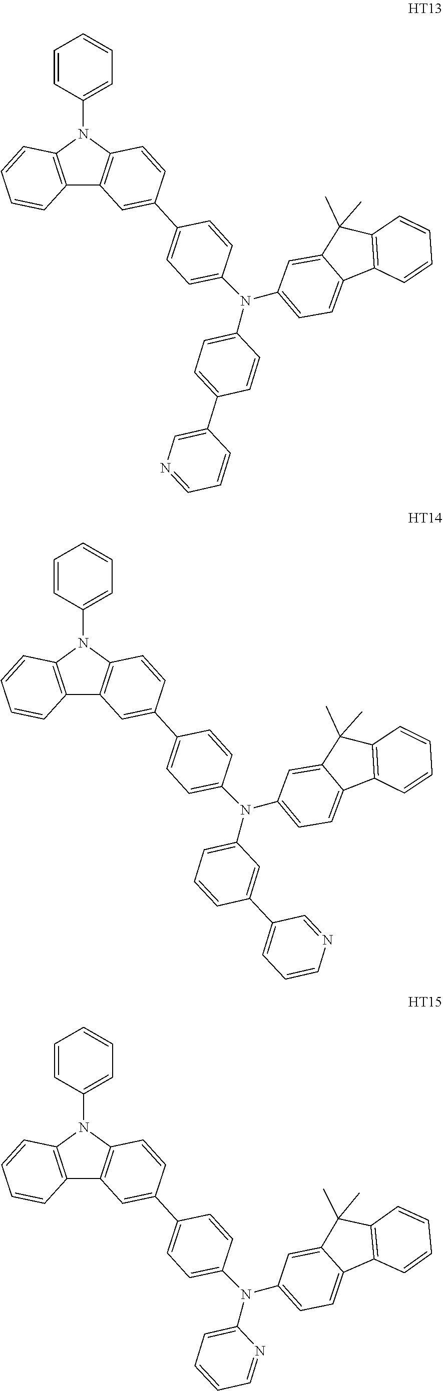 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

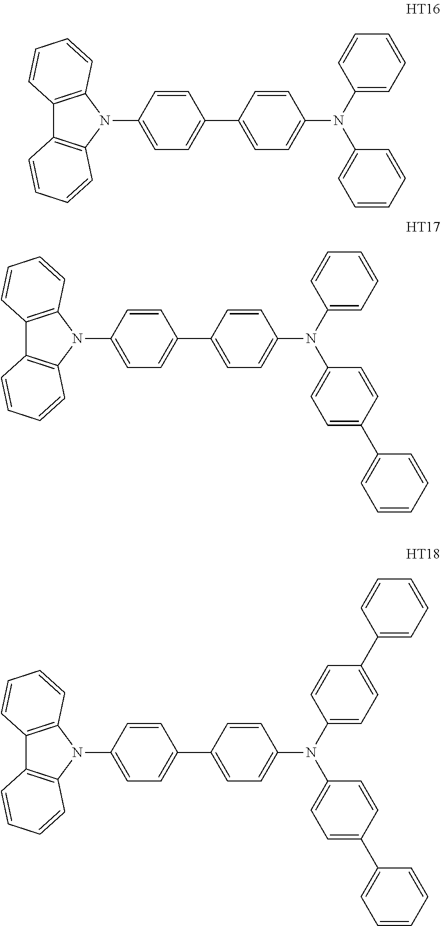

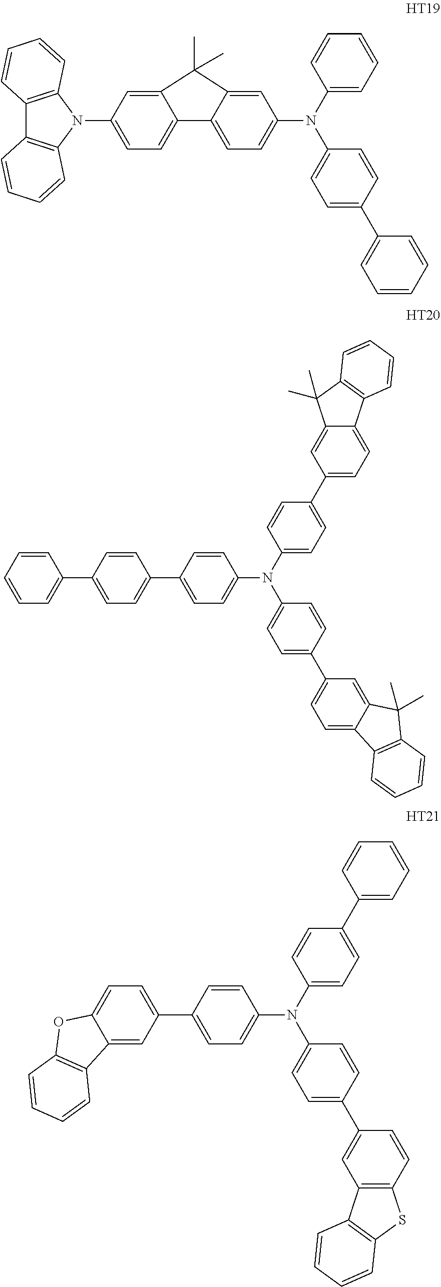

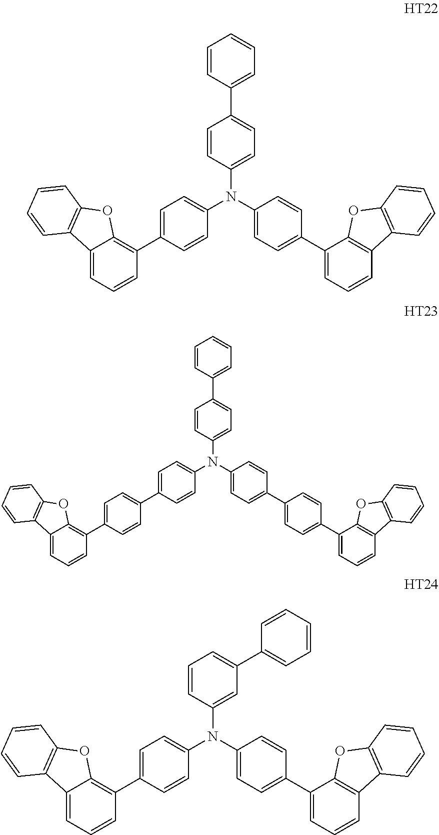 [SMILES: c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1]

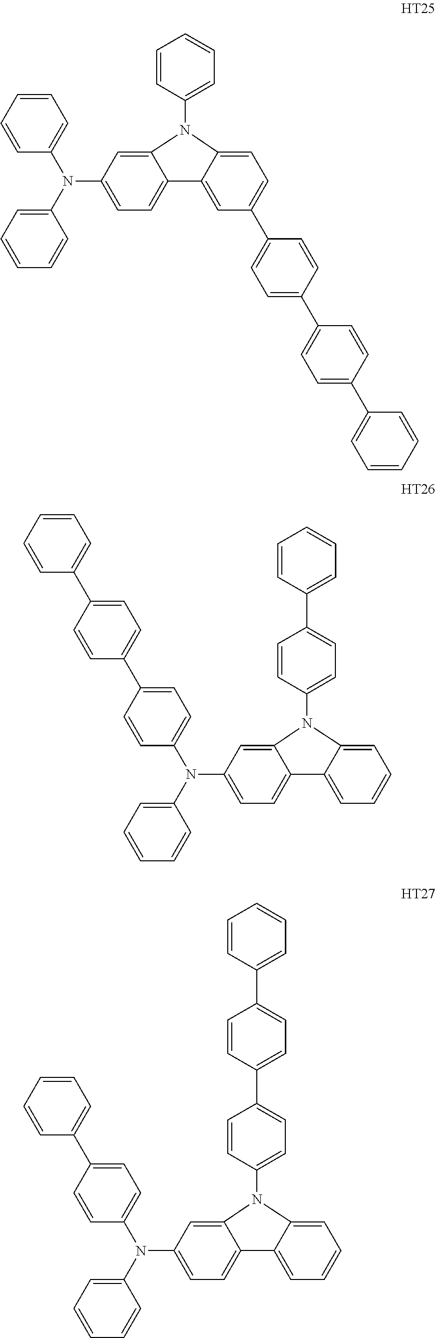 [SMILES: c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

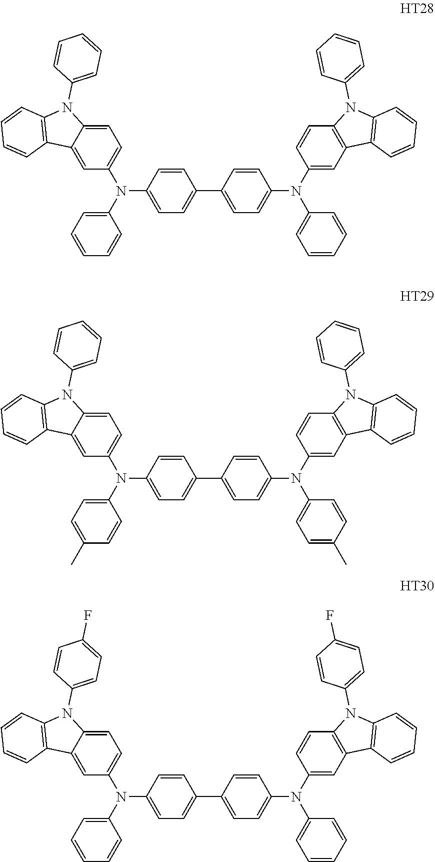 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

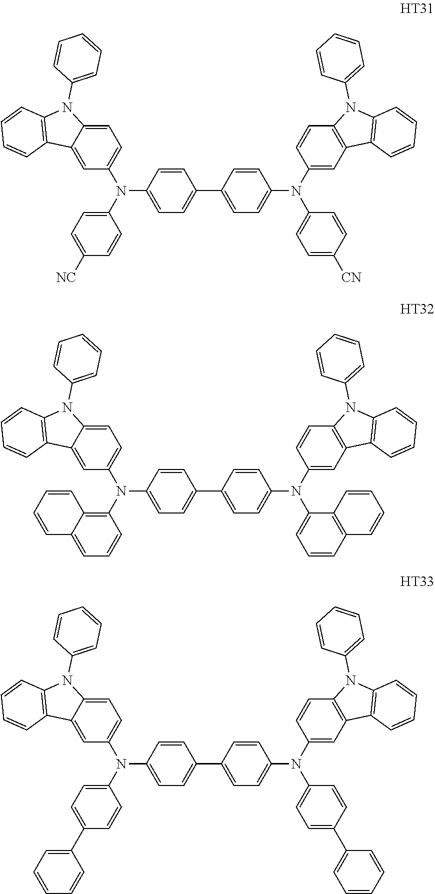 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

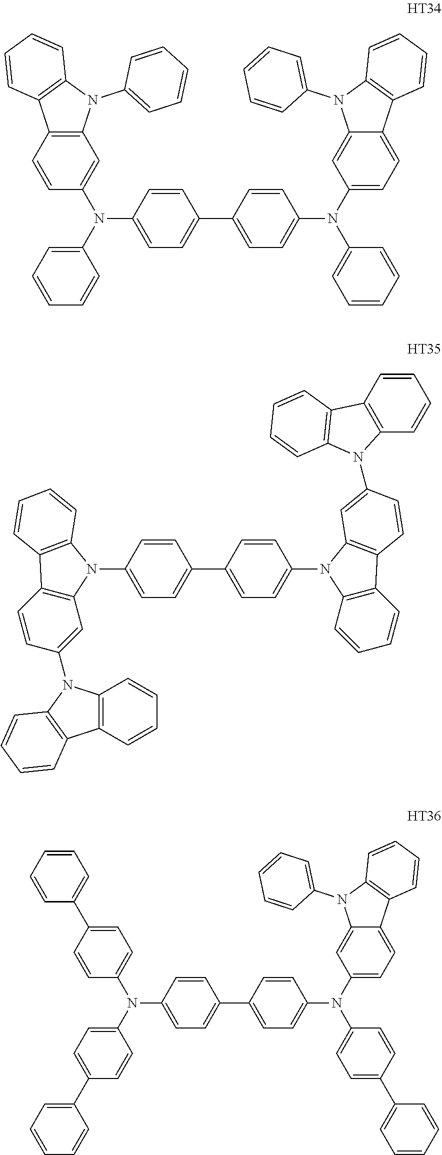 [SMILES: c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1 ; c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1]

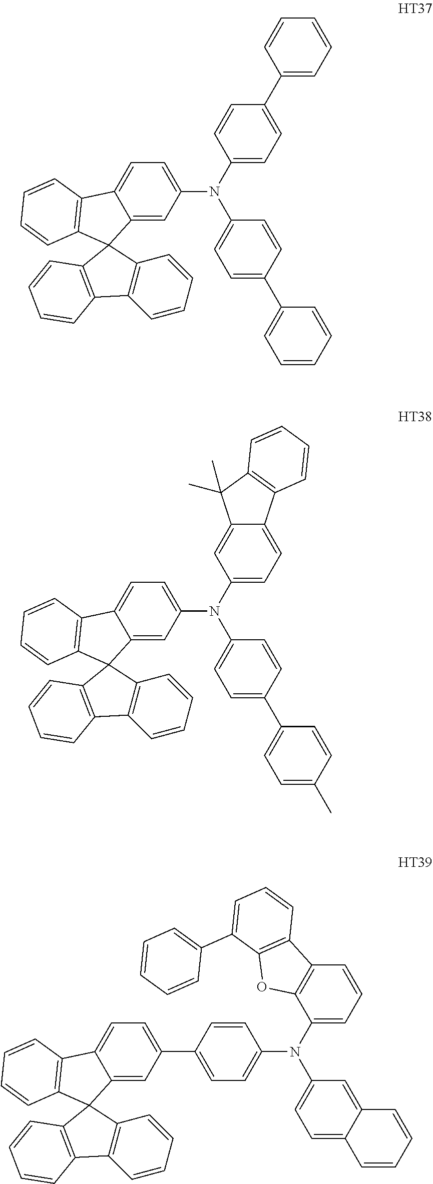 [SMILES: c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

In one or more embodiments, hole transport region 12 of the organic light-emitting device 10 may further include a p-dopant. When the hole transport region 12 further includes a p-dopant, the hole transport region 12 may have a matrix (for example, at least one of compounds represented by Formulae 201 to 205) and a p-dopant included in the matrix. The p-dopant may be uniformly or non-uniformly doped in the hole transport region 12.

In one or more embodiments, the LUMO energy level of the p-dopant may be −3.5 electron volts (eV) or less.

The p-dopant may include at least one of a quinone derivative, a metal oxide, or a cyano group-containing compound, but embodiments of the present disclosure are not limited thereto.

In one or more embodiments, the p-dopant may include at least one of:

a quinone derivative, such as tetracyanoquinodimethane (TCNQ), 2,3,5,6-tetrafluoro-7,7,8,8-tetracyanoquinodimethane (F4-TCNQ), or F6-TCNNQ;

a metal oxide, such as tungsten oxide or molybdenum oxide;

1,4,5,8,9,12-hexaazatriphenylene-hexacarbonitrile (HAT-CN); or

a compound represented by Formula 221 below,

but embodiments of the present disclosure are not limited thereto:

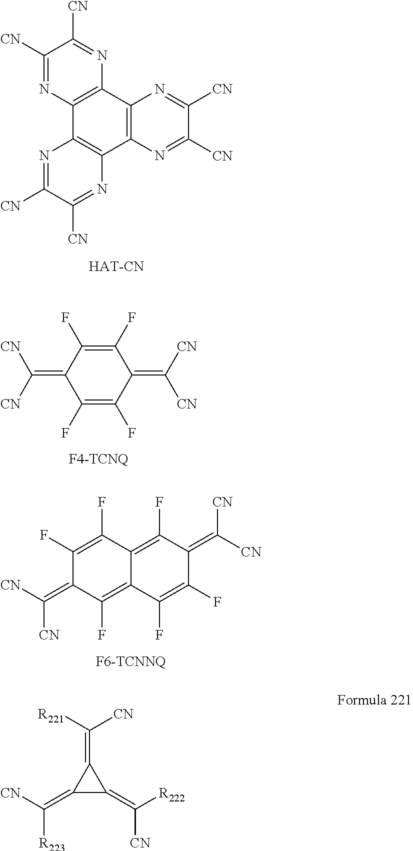

In Formula 221.

R₂₂₁ to R₂₂₃ may each independently be a substituted or unsubstituted C₃-C₁₀cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, and at least one of R₂₂₁ to R₂₂₃ may have at least one substituent that is a cyano group. —F, —Cl, —Br, —I, a C₁-C₂₀ alkyl group substituted with —F, a C₁-C₂₀ alkyl group substituted with —Cl, a C₁-C₂₀ alkyl group substituted with —Br, or a C₁-C₂₀ alkyl group substituted with —I.

The hole transport region 12 may have a thickness of about 100 Å to about 10000 Å, for example, about 400 Å to about 2000 Å, and the emission layer 15 may have a thickness of about 100 Å to about 3000 Å, for example, about 300 Å to about 1000 Å. When the thickness of each of the hole transport region 12 and the emission layer 15 is within these ranges described above, satisfactory hole transportation characteristics and/or luminescent characteristics may be obtained without a substantial increase in driving voltage.

### Electron Transport Region 17

The electron transport region 17 is placed between the emission layer 15 and the second electrode 19 of the organic light-emitting device 10.

The electron transport region 17 may have a single-layered structure or a multi-layered structure.

For example, the electron transport region 17 may have an electron transport layer, an electron transport layer/electron injection layer structure, a buffer layer/electron transport layer structure, hole-blocking layer/electron transport layer structure, a buffer layer/electron transport layer/electron injection layer structure, or a hole-blocking layer/electron transport layer/electron injection layer structure, but embodiments of the present disclosure are not limited thereto. The electron transport region 17 may further include an electron control layer.

The electron transport region 17 may include known electron-transporting materials.

The electron transport region (for example, a buffer layer, a hole-blocking layer, an electron control layer, or an electron transport layer in the electron transport region) may include a metal-free compound containing at least one T electron-deficient nitrogen-containing cyclic group. The n electron-deficient nitrogen-containing cyclic group is the same as described above.

In one or more embodiments, the electron transport region may include a compound represented by Formula 601 below:

[Ar₆₀₁]ₓ₆₁₁-[(L₆₀₁)ₓₑ₁-R₆₀₁]ₓₑ₂₁ Formula 601

wherein, in Formula 601,

Ar₆₀₁ and L₆₀₁ may each independently be a substituted or unsubstituted C₅-C₆₀ carbocyclic group or a substituted or unsubstituted C₁-C₆₀ heterocyclic group,

xe11 may be 1, 2, or 3,

xe1 is an integer from 0 to 5,

R₆₀₁ may be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), —P(Q₆₀₁)(Q₆₀₂), or —P(═O)(Q₆₀₁)(Q₆₀₂).

Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and

xe21 is an integer from 1 to 5.

In one or more embodiments, at least one of Ar₆₀₁(s) in the number of xe11 and R₆₀₁(s) in the number of xe21 may include the π electron-deficient nitrogen-containing cyclic group.

In one or more embodiments, ring Ar₆₀₁ and L₆₀₁ in Formula 601 may each independently be a phenyl group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one of deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), or —P(═O)(Q₃₁)(Q₃₂),

wherein Q₃₁ to Q₃₃ may each independently be a C₁-C₁₀ alkyl group, a C₁-C₁₀ alkoxy group, a phenyl group, a biphenyl group, a terphenyl group, or a naphthyl group.

When xe11 in Formula 601 is 2 or more, two or more of Ar₆₀₁(s) may be linked to each other via a single bond.

In one or more embodiments, Ar₆₀₁ in Formula 601 may be an anthracene group.

In one or more embodiments, the compound represented by Formula 601 may be represented by Formula 601-1:

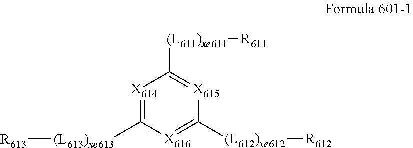

In Formula 601-1,

X₆₁₄ may be N or C(R₆₁₄), X₆₁₅ may be N or C(R₆₁₅), X₆₁₆ may be N or C(R₆₁₆), at least one of X₆₁₄ to X₆₁₆ may be N,

L₆₁₁ to L₆₁₃ may each independently be the same as described in connection with L₆₀₁,

xe611 to xe613 may each independently be the same as described in connection with xe1,

R₆₁₁ to R₆₁₃ may each independently be the same as described in connection with R₆₀₁, and

R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with at least one of deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group; or

—S(═O)₂(Q₆₀₁) or —P(═O)(Q₆₀₁)(Q₆₀₂),

wherein Q₆₀₁ and Q₆₀₂ are the same as described above.

The electron transport region may include at least one of Compounds ET1 to ET36, but embodiments of the present disclosure are not limited thereto:

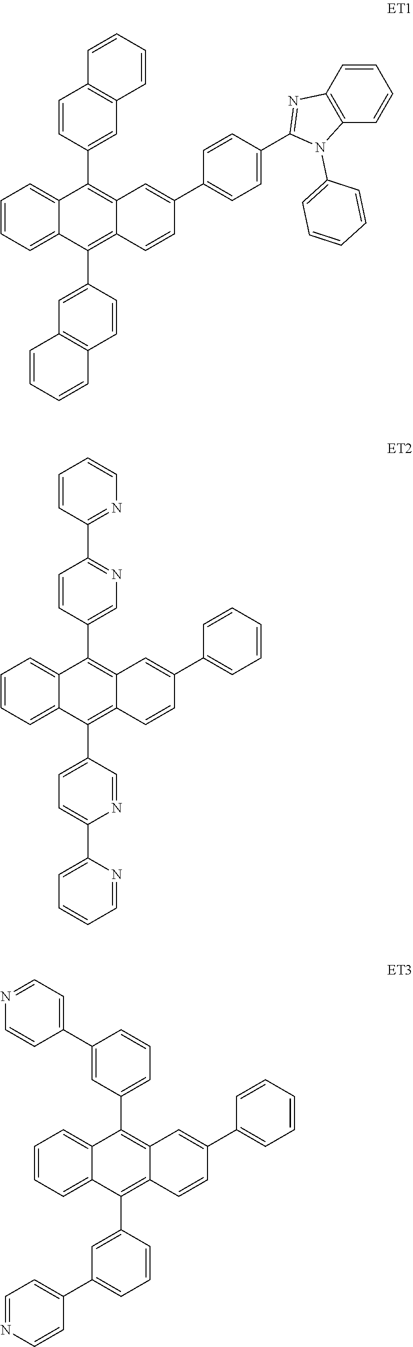 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

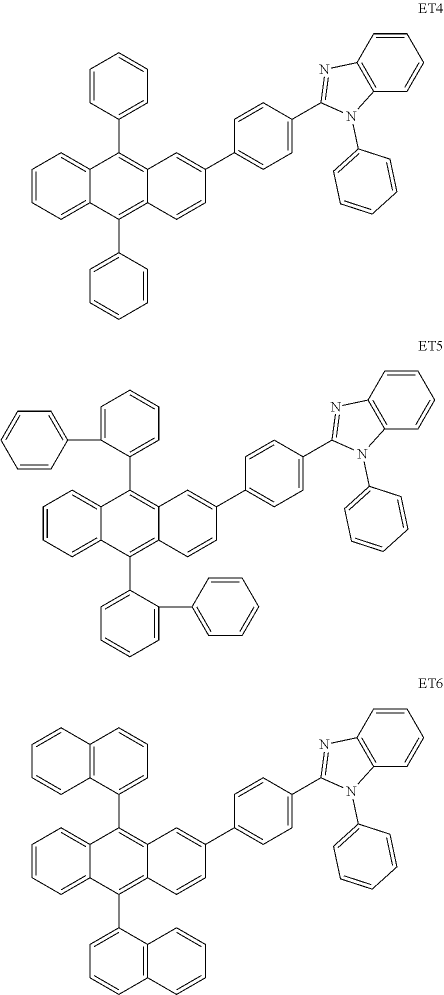 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

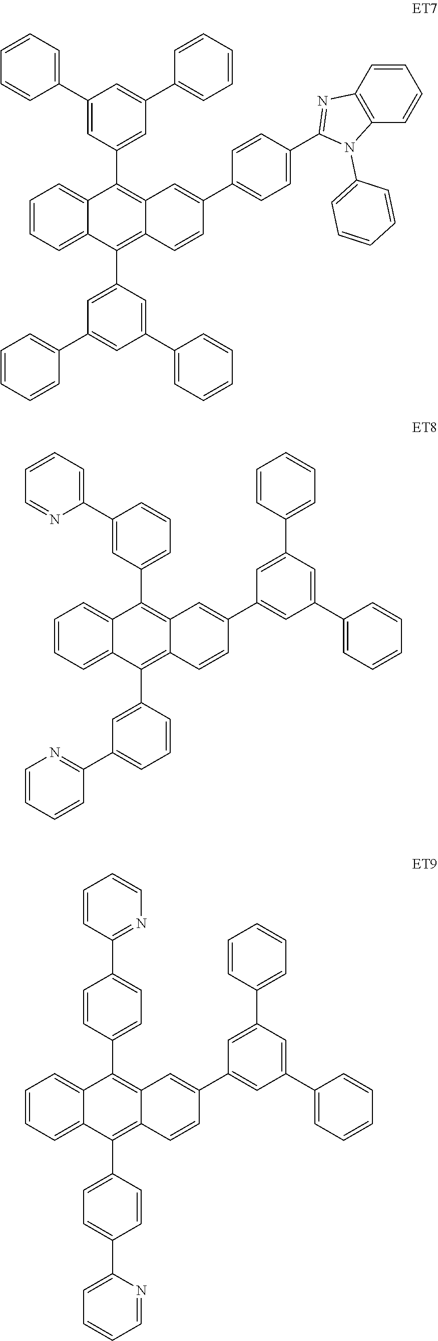 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

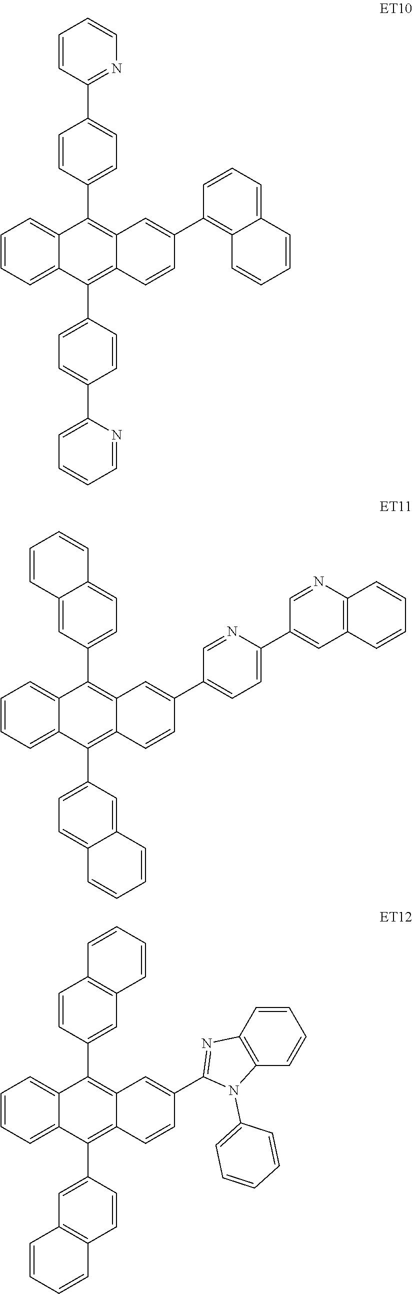

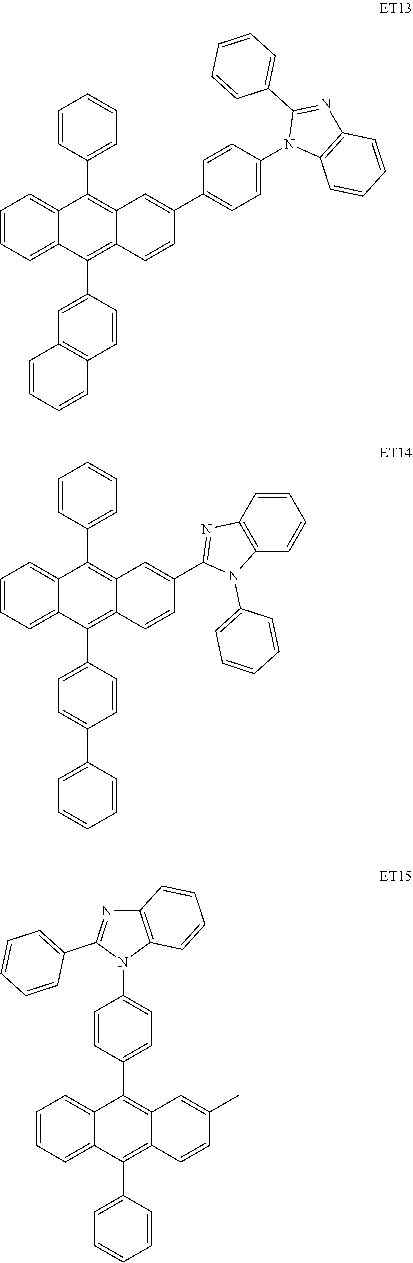 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

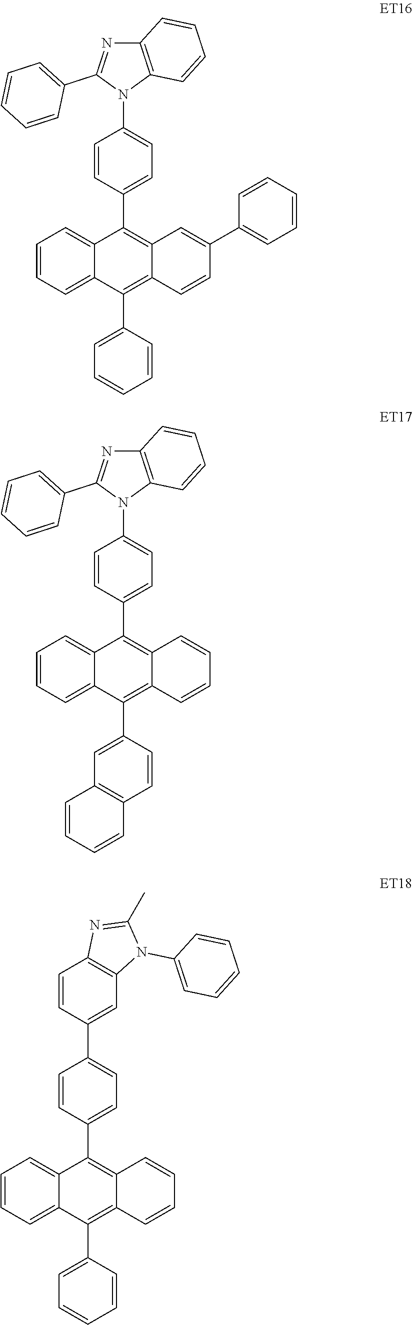 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

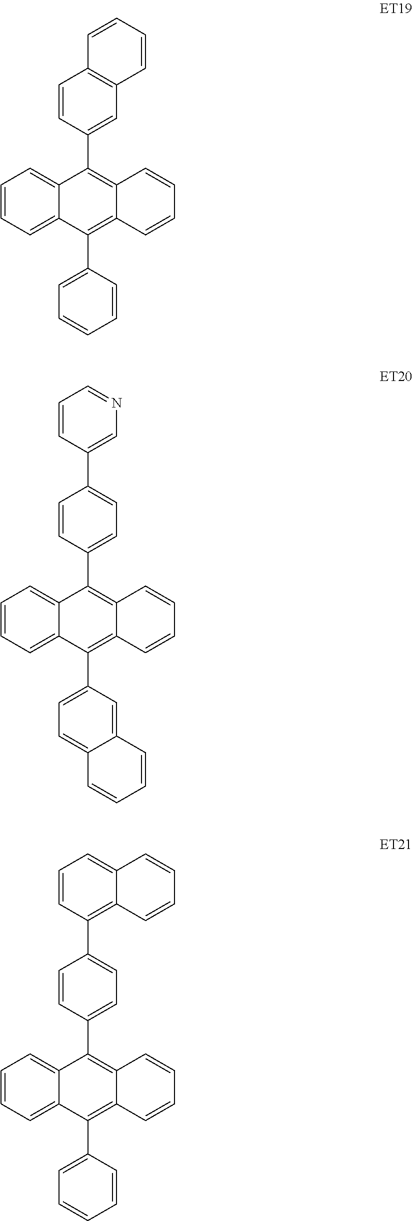

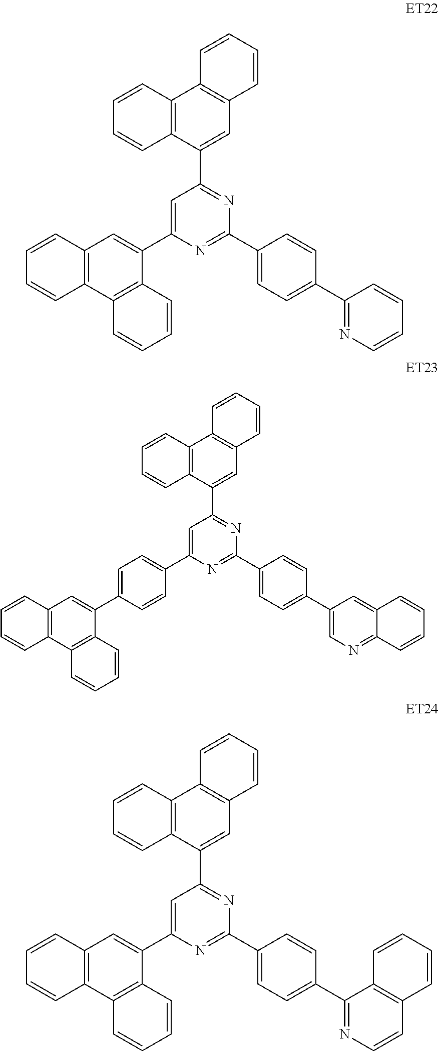 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

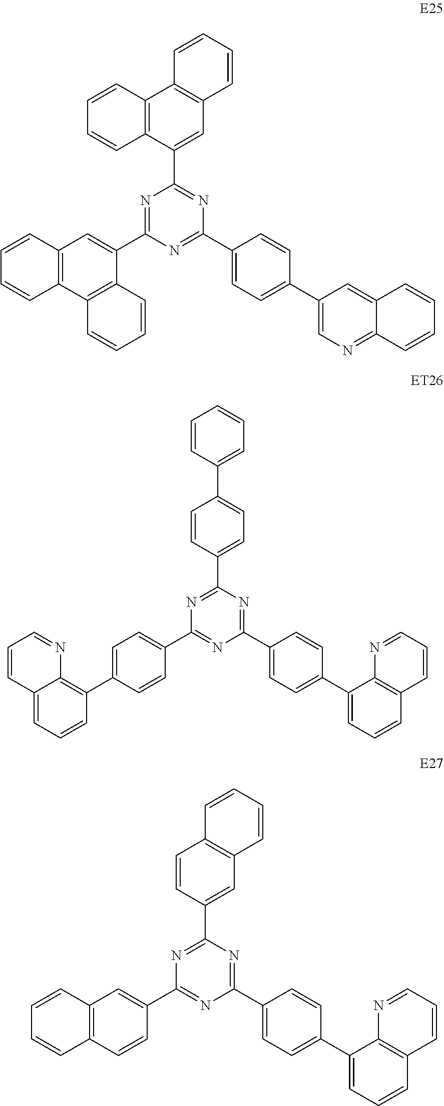 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

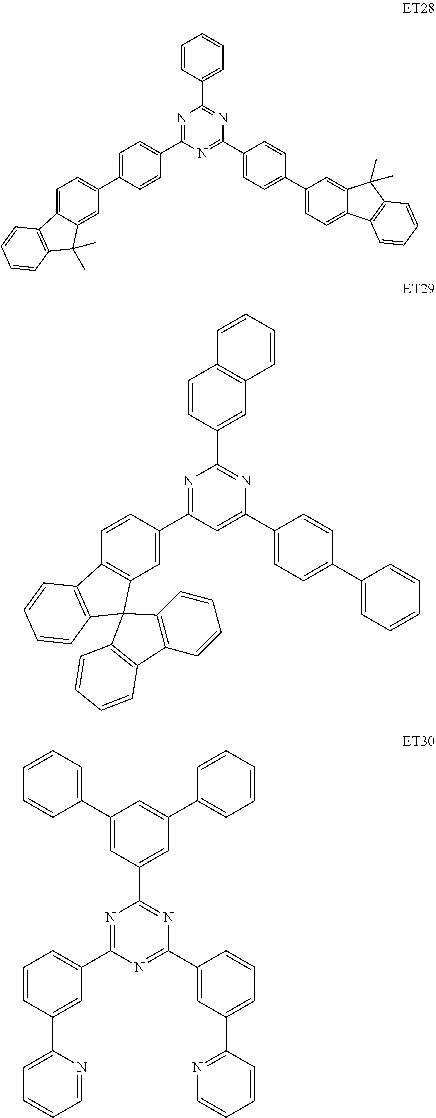 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

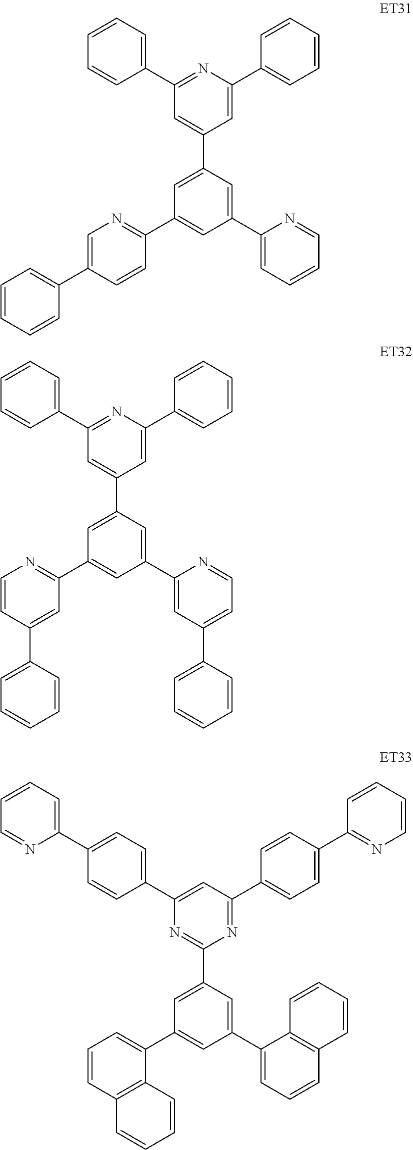

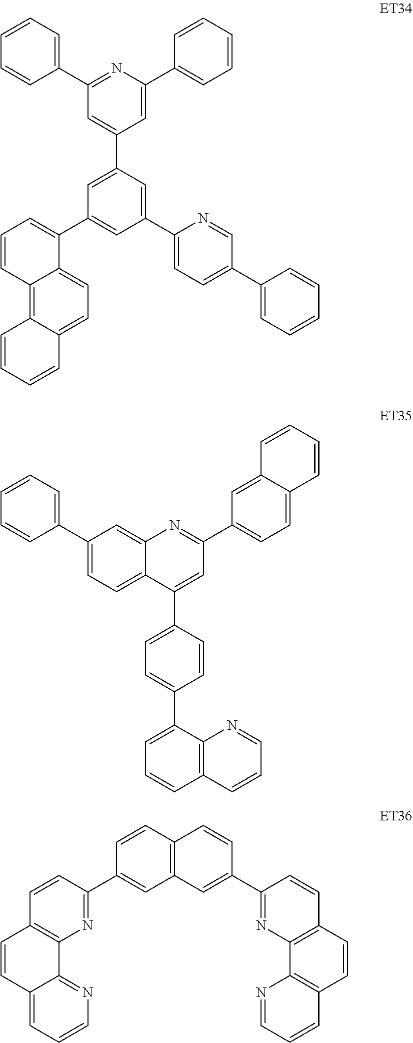

In one or more embodiments, the electron transport region may include at least one of 2,9-dimethyl-4,7-diphenyl-1,10-phenanthroline (BCP), 4,7-dphenyl-1,10-phenanthroline (Bphen), tris(8-hydroxyquinolinato)aluminum (Alq₃), bis(2-methyl-8-quinolinolato-N1,08)-(1,1′-biphenyl-4-olato)aluminum (BAlq), 3-(biphenyl-4-yl)-5-(4-tert-butylphenyl)-4-phenyl-4H-1,2,4-triazole (TAZ), or 4-(naphthalen-1-yl)-3,5-diphenyl-4H-1,2,4-triazole (NTAZ):

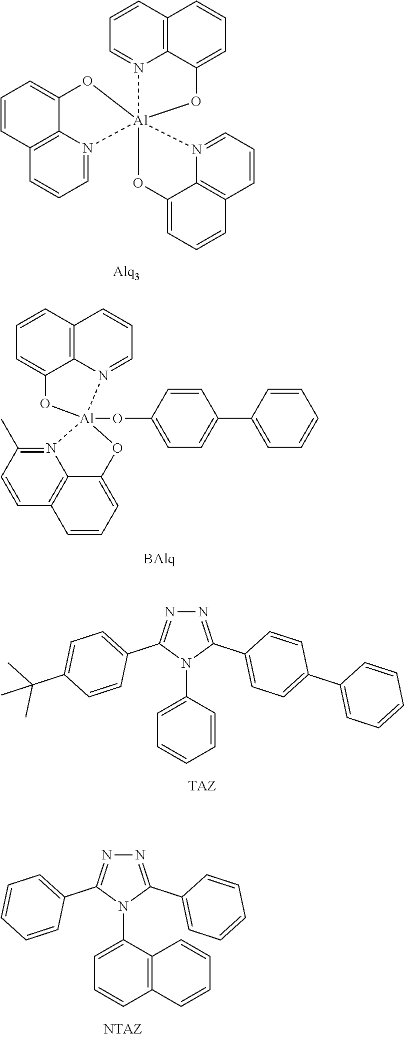

Thicknesses of the buffer layer, the hole-blocking layer, and the electron control layer may each independently be in the range of about 20 Å to about 1,000 Å, for example, about 30 Å to about 300 Å. When the thicknesses of the buffer layer, the hole-blocking layer, and the electron control layer are within these ranges, excellent hole blocking characteristics or excellent electron control characteristics may be obtained without a substantial increase in driving voltage.

A thickness of the electron transport layer may be in the range of about 100 Å to about 1,000 Å, for example, about 150 Å to about 500 Å. When the thickness of the electron transport layer is within the range described above, the electron transport layer may have satisfactory electron-transporting characteristics without a substantial increase in driving voltage.

The electron transport region 17 (for example, the electron transport layer in the electron transport region) may further include, in addition to the materials described above, a metal-containing material.

The metal-containing material may include at least one of an alkali metal complex or an alkaline earth-metal complex. The alkali metal complex may include a metal ion that is a Li ion, a Na ion, a K ion, a Rb ion, or a Cs ion, and the alkaline earth-metal complex may include a metal ion that is a Be ion, a Mg ion, a Ca ion, a Sr ion, or a Ba ion. A ligand coordinated with the metal ion of the alkali metal complex or the metal ion of the alkaline earth-metal complex may be a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazole, a hydroxy phenylpyridine, a hydroxy phenylbenzimidazole, a hydroxy phenylbenzothiazole, a bipyridine, a phenanthroline, or a cyclopentadiene, but embodiments of the present disclosure are not limited thereto.

In one or more embodiments, the metal-containing material may include a Li complex. The Li complex may include, for example, Compound ET-D1 (lithium quinolate,

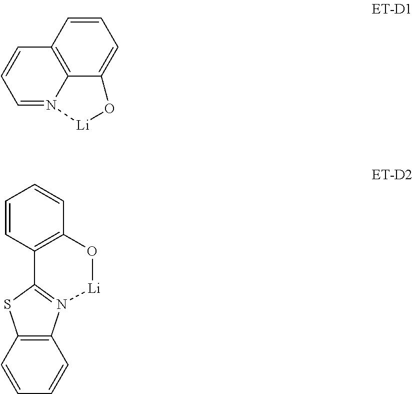

The electron transport region 17 may include an electron injection layer that facilitates the injection of electrons from the second electrode 19. The electron injection layer may directly contact the second electrode 19.

The electron injection layer may have i) a single-layered structure including a single layer including a single material, ii) a single-layered structure including a single layer including a plurality of different materials, or iii) a multi-layered structure having a plurality of layers including a plurality of different materials.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof.

The alkali metal may be Li, Na, K, Rb, or Cs. In one or more embodiments, the alkali metal may be Li, Na, or Cs. In one or more embodiments, the alkali metal may be Li or Cs, but embodiments of the present disclosure are not limited thereto.

The alkaline earth metal may be Mg, Ca. Sr, or Ba.

The rare earth metal may be Sc, Y, Ce, Tb, Yb, or Gd.

The alkali metal compound, the alkaline earth-metal compound, and the rare earth metal compound may be an oxide or a halide (for example, fluorides, chlorides, bromides, or iodides) of the alkali metal, the alkaline earth-metal, or the rare earth metal.

The alkali metal compound may be an alkali metal oxide, such as Li₂O, Cs₂O, or K₂O, or an alkali metal halide, such as LiF, NaF, CsF, KF, LiI, NaI, CsI, or KI. In one or more embodiments, the alkali metal compound may be LiF, Li₂O, NaF, LiI, NaI, CsI, or KI, but embodiments of the present disclosure are not limited thereto.

The alkaline earth-metal compound may be an alkaline earth-metal oxide, such as BaO, SrO, CaO, BaₓSr₁₋ₓO (0<x<1), or BaₓCa₁₋ₓO (0<x<1). In one or more embodiments, the alkaline earth-metal compound may be BaO, SrO, or CaO, but embodiments of the present disclosure are not limited thereto.

The rare earth metal compound may be YbF₃, ScF₃, Sc₂O₃, Y₂O₃, Ce₂O₃, GdF₃, or TbF₃. In one or more embodiments, the rare earth metal compound may be YbF₃, ScF₃, TbF₃, YbI₃, ScI₃, or TbI₃, but embodiments of the present disclosure are not limited thereto.

The alkali metal complex, the alkaline earth-metal complex, and the rare earth metal complex may include an ion of alkali metal, alkaline earth-metal, and/or rare earth metal as described above, and a ligand coordinated with a metal ion of the alkali metal complex, the alkaline earth-metal complex, and/or the rare earth metal complex may be hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, or cyclopentadiene, but embodiments of the present disclosure are not limited thereto.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof, as described above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof, may be homogeneously or non-homogeneously dispersed in a matrix including the organic material.

A thickness of the electron injection layer may be in a range of about 1 Å to about 100 Å, and, for example, about 3 Å to about 90 Å. When the thickness of the electron injection layer is within the range described above, the electron injection layer may have satisfactory electron injection characteristics without a substantial increase in driving voltage.

### Second Electrode 19

The second electrode 19 is located on the organic layer 10A having such a structure. The second electrode 19 may be a cathode which is an electron injection electrode, and in this regard, a material for forming the second electrode 19 may be a metal, an alloy, an electrically conductive compound, or a combination thereof, which have a relatively low work function.

The second electrode 19 may include at least one of lithium (Li), silver (Ag), magnesium (Mg), aluminum (Al), aluminum-lithium (Al—Li), calcium (Ca), magnesium-indium (Mg—In), magnesium-silver (Mg—Ag), ITO, or IZO, but embodiments of the present disclosure are not limited thereto. The second electrode 19 may be a transmissive electrode, a semi-transmissive electrode, or a reflective electrode.

The second electrode 19 may have a single-layered structure having a single layer or a multi-layered structure including two or more layers.

Hereinbefore, the organic light-emitting device has been described with reference to FIG. 1, but embodiments of the present disclosure are not limited thereto.

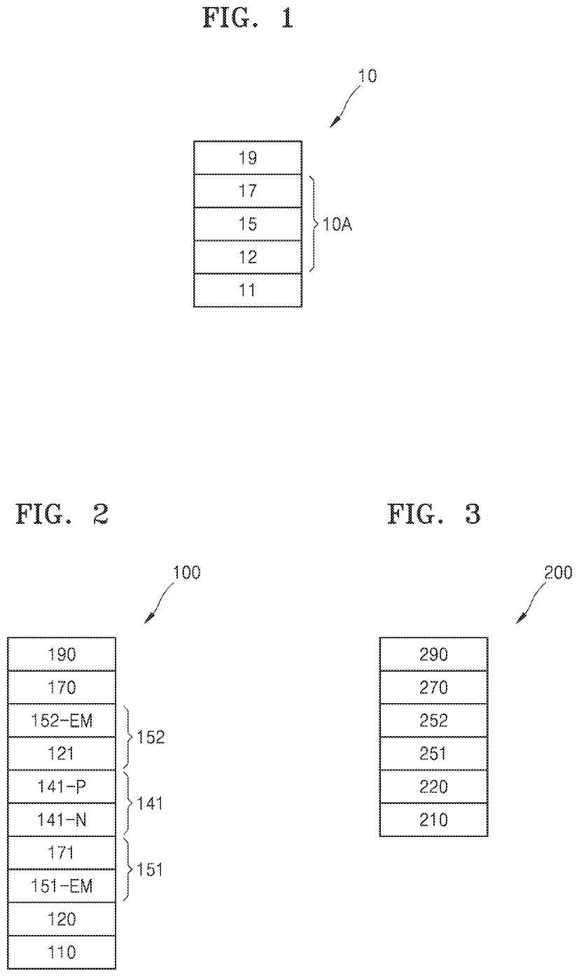

### Description of FIG. 2

FIG. 2 is a schematic cross-sectional view of an organic light-emitting device 100 according to another embodiment.

The organic light-emitting device 100 of FIG. 2 includes a first electrode 110, a second electrode 190 facing the first electrode 110, and a first light-emitting unit 151 and a second light-emitting unit 152 between the first electrode 110 and the second electrode 190. A charge generation layer 141 is located between the first emission unit 151 and the second emission unit 152, and the charge generation layer 141 may include an n-type charge generation layer 141-N and a p-type charge generation layer 141—P. The charge generation layer 141 is a layer that generates charge and supplies the charge to neighboring emission units, and any known material may be used therefor.

The first emission unit 151 may include a first emission layer 151-EM, and the second emission unit 152 may include a second emission layer 152-EM. The maximum emission wavelength of light emitted from the first emission unit 151 may be different from the maximum emission wavelength of light emitted from the second emission unit 152. For example, the mixed light of the light emitted from the first emission unit 151 and the light emitted from the second emission unit 152 may be white light, but embodiments of the present disclosure are not limited thereto.

The hole transport region 120 is located between the first emission unit 151 and the first electrode 110, and the second emission unit 152 may include the first hole transport region 121 located on the side of the first electrode 110.

An electron transport region 170 is located between the second emission unit 152 and the second electrode 190, and the first emission unit 151 may include a first electron transport region 171 located between the charge generation layer 141 and the first emission layer 151-EM.

The first emission layer 151-EM includes the composition described above.

For example, the first emission layer 151-EM may include a host, a dopant, and a sensitizer, the sensitizer may include the first compound of the composition, the dopant may include the second compound of the composition, and the host may include the third compound of the composition.

The second emission layer 152-EM includes the above composition.

For example, the second emission layer 152-EM may include a host, a dopant, and a sensitizer, the sensitizer may include the first compound of the composition, the dopant may include the second compound of the composition, and the host may include the third compound of the composition.

The first electrode 110 and the second electrode 190 illustrated in FIG. 2 may be the same as described in connection with the first electrode 11 and the second electrode 19 illustrated in FIG. 1.

The first emission layer 151-EM and the second emission layer 152-EM illustrated in FIG. 2 are each the same as described in connection with the emission layer 15 illustrated in FIG. 2.

The hole transport region 120 and the first hole transport region 121 illustrated in FIG. 2 are each the same as described in connection with the hole transport region 12 illustrated in FIG. 1.

The electron transport region 170 and the first electron transport region 171 illustrated in FIG. 2 are each the same as described in connection with the electron transport region 17 illustrated in FIG. 1.

As described above, referring to FIG. 2, an organic light-emitting device in which each of the first light-emitting unit 151 and the second light-emitting unit 152 includes an emission layer including a host, a dopant, and a sensitizer, has been described. However, the organic light-emitting device may have various other forms. For example, one of the first light-emitting unit 151 and the second light-emitting unit 152 of the organic light-emitting device 100 of FIG. 2 may be replaced with any known light-emitting unit, or may include three or more light-emitting units.

### Description of FIG. 3

FIG. 3 is a schematic cross-sectional view of an organic light-emitting device 200 according to another embodiment.

The organic light-emitting device 200 includes a first electrode 210, a second electrode 290 facing the first electrode 210, and a first emission layer 251 and a second emission layer 252 which are stacked between the first electrode 210 and the second electrode 290.

The maximum emission wavelength of light emitted from the first emission layer 251 may be different from the maximum emission wavelength of light emitted from the second emission layer 252. For example, the mixed light of the light emitted from the first emission layer 251 and the light emitted from the second emission layer 252 may be white light, but embodiments of the present disclosure are not limited thereto.

In one or more embodiments, a hole transport region 220 may be located between the first emission layer 251 and the first electrode 210, and an electron transport region 270 may be located between the second emission layer 252 and the second electrode 290.

The first emission layer 251 includes the composition described above.

For example, the first emission layer 251 may include a host, a dopant, and a sensitizer, the sensitizer may include the first compound of the composition, the dopant may include the second compound of the composition, and the host may include the third compound of the composition.

The second emission layer 252 includes the composition described above.

For example, the second emission layer 252 may include a host, a dopant, and a sensitizer, the sensitizer may include the first compound of the composition, the dopant may include the second compound of the composition, and the host may include the third compound of the composition.

The first electrode 210, the hole transport region 220, and the second electrode 290 illustrated in FIG. 3 are respectively the same as described in connection with the first electrode 11, the hole transport region 12, and the second electrode 19 illustrated in FIG. 1.

The first emission layer 251 and the second emission layer 252 illustrated in FIG. 3 are each the same as described in connection with the emission layer 15 illustrated in FIG. 1.

The electron transport region 270 illustrated in FIG. 3 may be the same as described in connection with the electron transport region 17 in FIG. 1.

As described above, referring to FIG. 3, an organic light-emitting device, in which each of the first emission layer 251 and the second emission layer 252 includes a host, a dopant, and a sensitizer, has been described. However, the organic light-emitting device may have various other forms. For example, one of the first emission layer 251 and the second emission layer 252 of the organic light-emitting device 200 of FIG. 3 may be replaced with any known emission layer, or an interlayer may be additionally located between neighboring emission layers.

### Explanation of Terms

The term “C₁-C₆₀ alkyl group” as used herein refers to a linear or branched saturated aliphatic hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is a C₁-C₆₀ alkyl group), and examples thereof include a methoxy group, an ethoxy group, and an isopropyloxy group. The term “C₁-C₆₀ alkylthio group” as used herein refers to a monovalent group represented by —SA₁₀₁ (wherein A₁₀₁ is a C₁-C₆₀ alkyl group).

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom that is N, O, P, Si, or S as a ring-forming atom and 1 to 10 carbon atoms, and non-limiting examples thereof include a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom that is N, O, P, Si, or S as a ring-forming atom, 1 to 10 carbon atoms, and at least one double bond in its ring. Examples of the C₁-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₇-C₆₀ alkyl aryl group” as used herein refers to a C₆₋₆₀ aryl group that is substituted with a C₁₋₆₀ alkyl group. The term “C₇-C₆₀ aryl alkyl group” as used herein refers to a C₁₋₆₀ alkyl group that is substituted with a C₆₋₆₀ aryl group.

The term “C₁-C₆₀ heteroaryl group” as used herein refers to a monovalent group having a heterocarbocyclic aromatic system that has at least one heteroatom that is N, O, P, Si, or S as a ring-forming atom, and 1 to 60 carbon atoms. The term “C₁-C₆₀ heteroarylene group” as used herein refers to a divalent group having a carbocyclic aromatic system that has at least one heteroatom that is N, O, P, Si, or S as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₆-C₆₀ heteroarylene group each include two or more rings, the rings may be fused to each other.

The term “C₂-C₆₀ alkyl heteroaryl group” as used herein refers to a C₁₋₆₀ heteroaryl group that is substituted with a C₁₋₆₀ alkyl group. The term “C₂-C₆₀ heteroaryl alkyl group” as used herein refers to a C₁₋₆₀ alkyl group that is substituted with a C₁₋₆₀ heteroaryl group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group). The term “C₁-C₆₀ heteroaryloxy group” as used herein indicates —OA₁₀₄ (wherein A₁₀₄ is the C₁-C₆₀ heteroaryl group), and the term “C₆-C₆₀ heteroarylthio group” as used herein indicates —SA₁₀₅ (wherein A₁₀₅ is the C₁-C₆₀ heteroaryl group).

The term “monovalent non-aromatic condensed polycyclic group” used herein refers to a monovalent group in which two or more rings are condensed with each other, only carbon is used as a ring-forming atom (for example, the number of carbon atoms may be 8 to 60) and the whole molecule is a non-aromaticity group. Examples of the monovalent non-aromatic condensed polycyclic group include a fluorenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group having two or more rings condensed to each other, a heteroatom that is N, O, P. Si, or S, other than carbon atoms (for example, having 1 to 60 carbon atoms), as a ring-forming atom, and no aromaticity in its entire molecular structure. Examples of the monovalent non-aromatic condensed heteropolycyclic group include a carbazolyl group. The term “divalent non-aromatic heterocondensed polycyclic group” as used herein refers to a divalent group having the same structure as a monovalent non-aromatic heterocondensed polycyclic group.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group, and may be a monovalent, divalent, trivalent, tetravalent, pentavalent, or hexavalent group, depending on the formula structure.

The term “C₁-C₃₀ heterocyclic group” as used herein refers to a saturated or unsaturated cyclic group having, as a ring-forming atom, at least one heteroatom that is N, O, P, Si, or S other than 1 to 30 carbon atoms. The C₁-C₆₀ heterocyclic group may be a monocyclic group or a polycyclic group, and may be a monovalent, divalent, trivalent, tetravalent, pentavalent, or hexavalent group, depending on the formula structure.

At least one substituent of the substituted C₅-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(Q₁₈)(Q₁₉)—, —P(═S)(Q₁₈)(Q₁₉), or —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a C₁-C₆₀ heteroaryloxy group, a C₁-C₆₀ heteroarylthio group, or a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each substituted with at least one of deuterium. —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₂-C₆₀ aryl alkyl group, a C₂-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(Q₂₈)(Q₂₉), —P(═S)(Q₂₈)(Q₂₉), or —P(═O)(Q₂₈)(Q₂₉); or

—N(Q₃₁)(Q₃₂), —Si(Q₃₃)(Q₃₄)(Q₃₅), —B(Q₃₆)(Q₃₇), P(Q₂₈)(Q₂₉), —P(═S)(Q₂₈)(Q₂₉), or —P(═O)(Q₃₈)(Q₃₉),

wherein Q₁ to Q₉, Q₁₁ to Q₁₉, Q₂₁ to Q₂₉ and Q₃₁ to Q₃₉ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group a C₁-C₆₀ alkylthio group, or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of a C₁-C₆₀ alkyl group or a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group.

The term “room temperature” used herein refers to a temperature of about 25° C.

The terms “a biphenyl group, a terphenyl group, and a quaterphenyl group” as used herein respectively refer to monovalent groups in which two, three, or four phenyl groups which are linked together via a single bond.

The terms “a cyano-containing phenyl group, a cyano-containing biphenyl group, a cyano-containing terphenyl group, and a cyano-containing quaterphenyl group” as used herein respectively refer to a phenyl group, a biphenyl group, a terphenyl group, and a quaterphenyl group, each of which is substituted with at least one cyano group. In “a cyano-containing phenyl group, a cyano-containing biphenyl group, a cyano-containing terphenyl group, and a cyano-containing quaterphenyl group”, a cyano group may be substituted to any position of the corresponding group, and the “cyano-containing phenyl group, the cyano-containing biphenyl group, the cyano-containing terphenyl group, and the cyano-containing quaterphenyl group” may further include substituents other than a cyano group. For example, a phenyl group substituted with a cyano group, and a phenyl group substituted with a cyano group and a methyl group may all belong to “a cyano-containing phenyl group.”

Hereinafter, a compound and an organic light-emitting device according to one or more exemplary embodiments are described in further detail with reference to Synthesis Examples and Examples. However, the organic light-emitting device is not limited thereto. The wording ‘“B’ was used instead of ‘A”’ used in describing Synthesis Examples means that an amount of ‘A’ used was identical to an amount of ‘B’ used, in terms of a molar equivalent.

### EXAMPLES

### Example 1-1

An ITO glass substrate was cut to a size of 50 millimeters (mm)×50 mm×0.5 mm and then, sonicated in acetone isopropyl alcohol and deionized (DI) water, each for 15 minutes, and then, cleaned by exposure to ultraviolet (UV) light and ozone for 30 minutes.

Subsequently, F6-TCNNQ was deposited on the ITO electrode (anode) on the glass substrate to form a hole injection layer having a thickness of 100 Å, and HT1 was deposited on the hole injection layer to form a hole transport layer having a thickness of 1260 Å, thereby completing the manufacture of a hole transport region

Compound H-H104 (hereinafter referred to as H-H1 ((first host), Compound 57 of Group HE4 (hereinafter referred to as H-E1)(second host), Compound SP001(sensitizer)(where the weight ratio of the first host, the second host, and the sensitizer is 45:45:10), and FD1(dopant)(where the amount of the dopant is 0.1 weight percent (wt %) based on the total weight of the first host, the second host, the sensitizer, and the dopant) were co-deposited on the hole transport region to form an emission layer having a thickness of 400 Å.

Compound ET17 and Liq were co-deposited at the weight ratio of 5:5 on the emission layer to form an electron transport layer having a thickness of 360 Å, and then, LiQ was deposited on the electron transport layer to form an electron injection layer having a thickness of 5 Å, and Al was deposited on the electron injection layer to form a cathode having a thickness of 800 Å, thereby completing the manufacture of an organic light-emitting device.

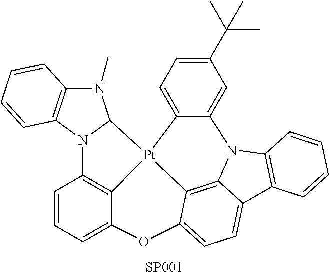

### Examples 1-2 to 1-3, Examples 2-1 to 2-3, Example 3-1, Comparative Example 1, Comparative Examples 1-1 to 1-3, Comparative Example 2, Comparative Examples 2-1 to 2-3, Comparative Example 3, and Comparative Example 3-1

Organic light-emitting devices were manufactured in the same manner as in Example 1-1, except that, in forming an emission layer, for use as a sensitizer and a dopant, the compounds and amounts shown in Table 7 were used.

TABLE 7

| | | | | | Dopant |
|---|---|---|---|---|---|
| | | Second | | | amount |
| | First host | host | Sensitizer | Dopant | (wt %) |
| Comparative | H-H1 | H-E1 | SP001 | — | — |
| Example 1 | | | | | |
| Example 1-1 | H-H1 | H-E1 | SP001 | FD1 | 0.1 |
| Example 1-2 | H-H1 | H-E1 | SP001 | FD1 | 0.5 |
| Example 1-3 | H-H1 | H-E1 | SP001 | FD1 | 1.5 |
| Comparative | H-H1 | H-E1 | SP001 | FD-A | 0.1 |
| Example 1-1 | | | | | |
| Comparative | H-H1 | H-E1 | SP001 | FD-A | 0.5 |
| Example 1-2 | | | | | |
| Comparative | H-H1 | H-E1 | SP001 | FD-A | 1.5 |
| Example 1-3 | | | | | |
| Comparative | H-H1 | H-E1 | SP002 | — | — |
| Example 2 | | | | | |
| Example 2-1 | H-H1 | H-E1 | SP002 | FD1 | 0.1 |
| Example 2-2 | H-H1 | H-E1 | SP002 | FD1 | 0.5 |
| Example 2-3 | H-H1 | H-E1 | SP002 | FD1 | 1.5 |
| Comparative | H-H1 | H-E1 | SP002 | FD-A | 0.1 |
| Example 2-1 | | | | | |
| Comparative | H-H1 | H-E1 | SP002 | FD-A | 0.5 |
| Example 2-2 | | | | | |
| Comparative | H-H1 | H-E1 | SP002 | FD-A | 1.5 |
| Example 2-3 | | | | | |
| Comparative | H-H1 | H-E1 | SP003 | — | — |
| Example 3 | | | | | |
| Example 3-1 | H-H1 | H-E1 | SP003 | FD2 | 1.5 |
| Comparative | H-H1 | H-E1 | SP003 | FD-B | 1.5 |
| Example 3-1 | | | | | |

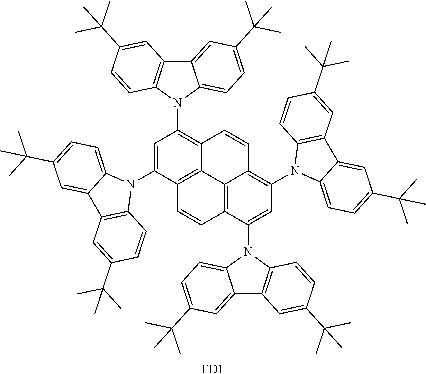

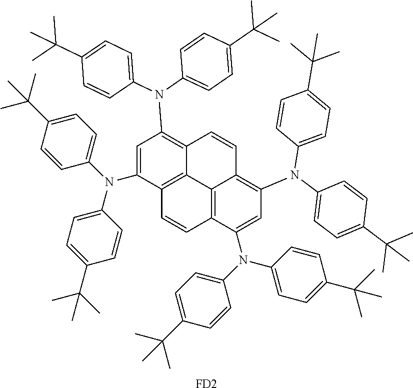

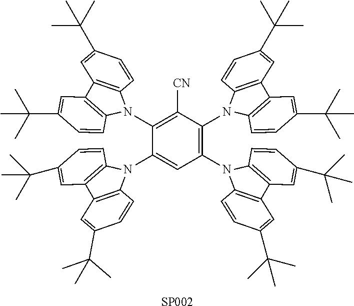

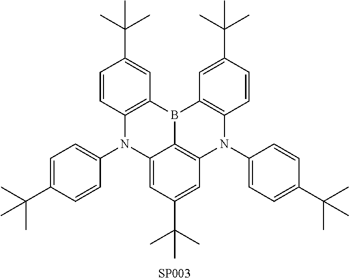

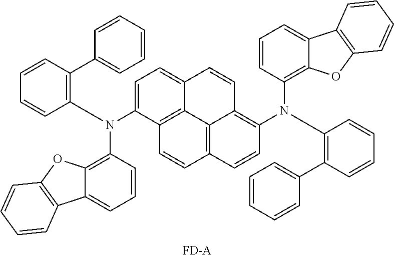 [SMILES: c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; "FD-A"]

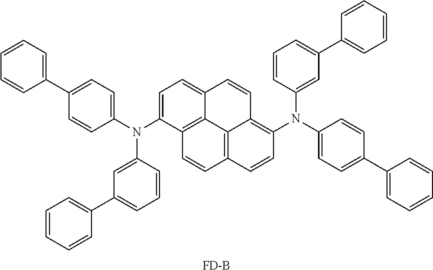 [SMILES: c1ccc(-c2ccc(N(c3cccc(-c4ccccc4)c3)c3ccc4ccc5c(N(c6ccc(-c7ccccc7)cc6)c6cccc(-c7ccccc7)c6)ccc6ccc3c4c65)cc2)cc1 ; "FD-B"]

### Evaluation Example 1. ΔE_{ST} of Compounds SP002 and SP003

The S1 and T₁ energy levels of the compounds SP002 and SP003 were measured according to the method described in Table 8, and the results are shown in Table 9

TABLE 8

| | |
|---|---|
| T1 Energy | Put a mixture of toluene and each compound (dissolve |
| Level | each compound to a concentration of 1 × 10−4 M in 3 mL |
| Assessment | of toluene) into a quartz cell, add liquid nitrogen (77 K), |
| Method | and use a photoluminescence measuring instrument to |
| | detect photoluminescence. The onset T1 energy level is |
| | calculated by measuring the spectrum and comparing it |
| | with the normal room temperature photoluminescence |
| | spectrum and analyzing only the peak observed only at |
| | low temperature. |
| S1 Energy | The onset S1 energy level was calculated by measuring the |
| Level | photoluminescence spectrum of a mixture of toluene and |
| Assessment | each compound (diluted to a concentration of 1 × 10−4 M) |
| Method | at room temperature using a photoluminescence measuring |
| | instrument and analyzing the observed peak. |

TABLE 9

| Compound | ΔE_{ST} (peak reference) | ΔE_{ST} (onset reference) |
|---|---|---|
| SP002 | 0.11 eV | 0.15 eV |
| SP003 | 0.329 eV | 0.4 eV |

### Evaluation Example 2: Measurement of OLED Lifespan and External Quantum Efficiency

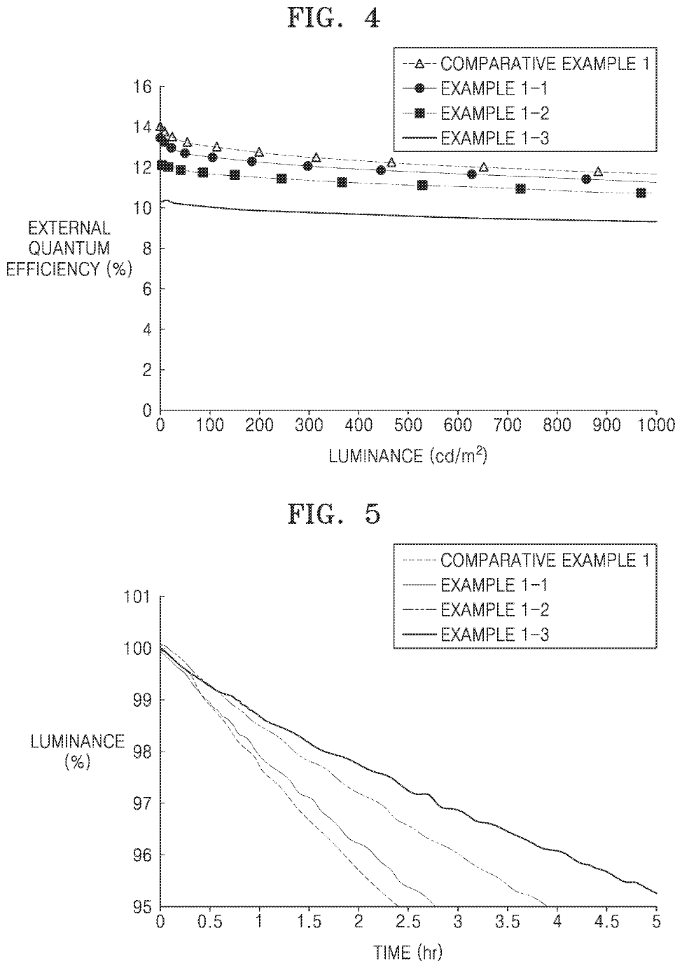

(1) For each of the organic light-emitting devices manufactured according to Comparative Example 1, Examples 1-1 to 1-3, and Comparative Examples 1-1 to 1-3, external quantum efficiency (EQE) and lifespan were evaluated. Results thereof are shown in FIGS. 4 to 7.

First, referring to FIGS. 4 and 5, in the case of Examples 1-1 to 1-3, some of the triplet excitons generated in SP001, which is a phosphorescent sensitizer, were lost by FD1, and the efficiency was slightly reduced compared to Comparative Example 1. However, the lifespan thereof was significantly increased.

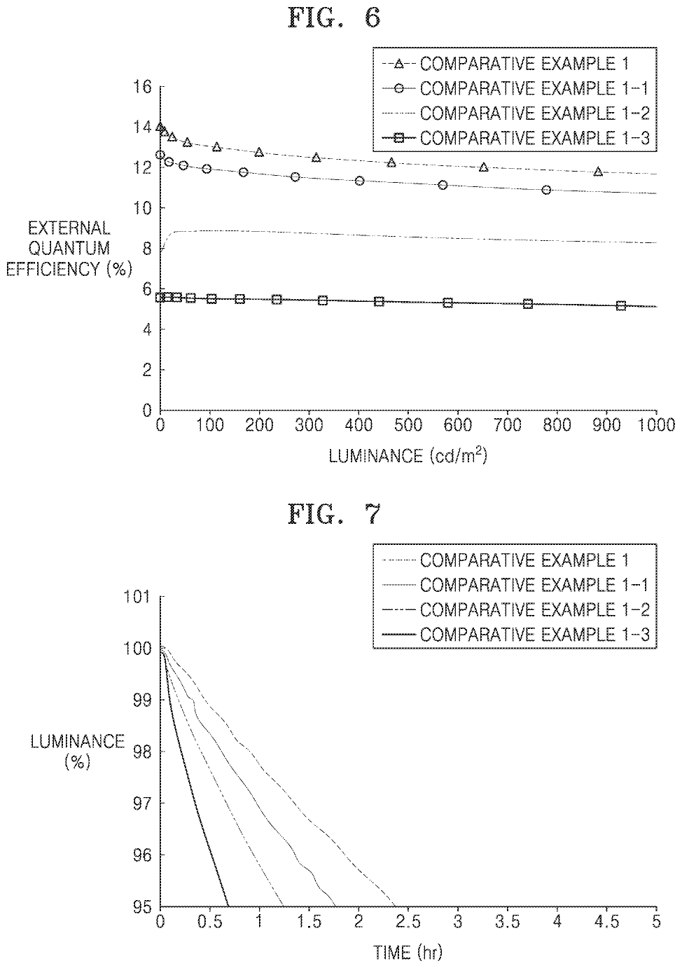

On the other hand, referring to FIGS. 6 and 7, in the case of Comparative Examples 1-1 to 1-3, even compared to Examples 1-1 to 1-3 in which the same amount of dopant was included, the reduction in external quantum efficiency was remarkable, and rather, the lifespan was significantly reduced compared to Comparative Example 1. This is because triplet excitons generated in SP001 through dexter energy transfer are transferred to FD-A, and thus, do not contribute to light emission, and deteriorate due to non-radiative decay.

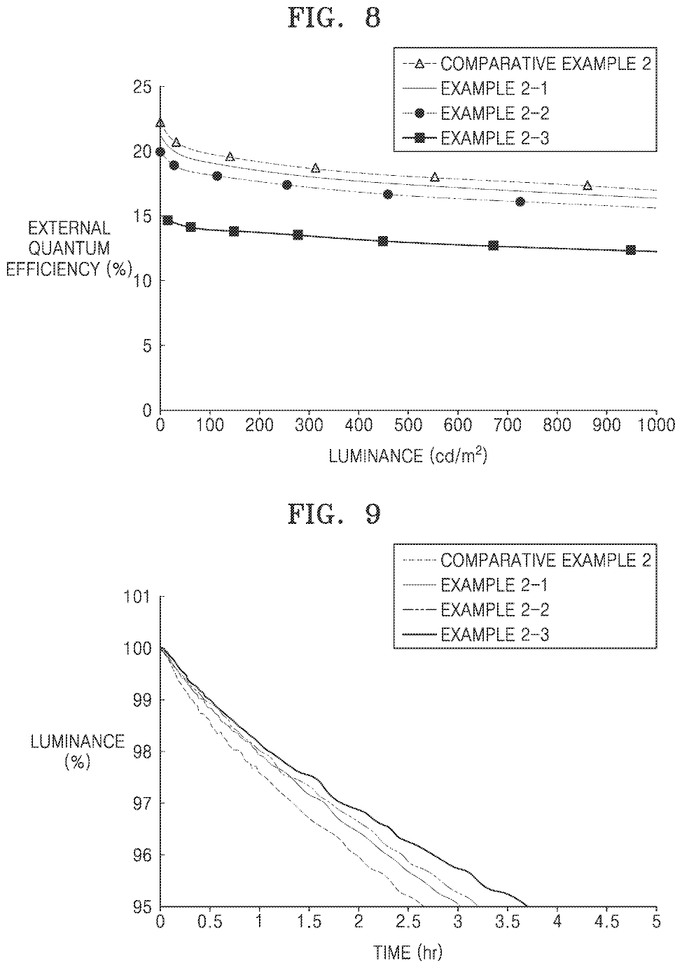

(2) For each of the organic light-emitting devices manufactured according to Comparative Example 2, Examples 2-1 to 2-3, and Comparative Examples 2-1 to 2-3, external quantum efficiency (EQE) and lifespan were evaluated. Results thereof are shown in FIGS. 8 to 11.

First, referring to FIGS. 8 and 9, in the case of Examples 2-1 to 2-3, some of the triplet excitons generated in SP002, which is a TADF sensitizer, was lost by FD1, and the efficiency was slightly reduced compared to Comparative Example 2 However, the lifespan thereof was significantly increased.

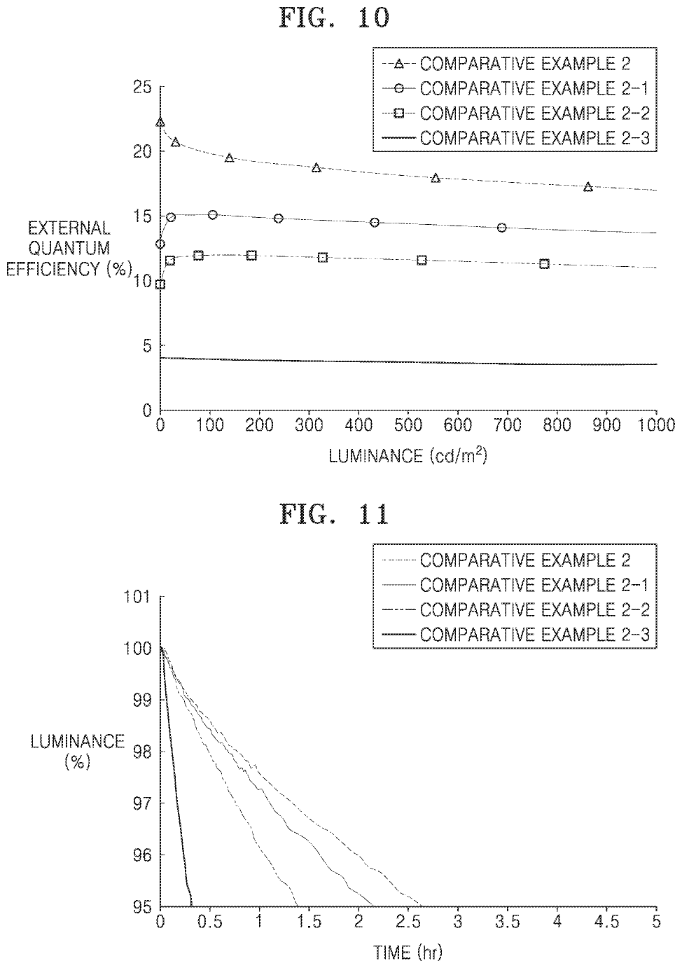

On the other hand, referring to FIGS. 10 and 11, in the case of Comparative Examples 2-1 to 2-3, even compared to Examples 2-1 to 2-3 in which the same amount of dopant was included, the reduction in external quantum efficiency was remarkable, and rather, the lifespan was significantly reduced compared to Comparative Example 1. This is because triplet excitons generated in SP002 through dexter energy transfer are transferred to FD-A, and thus, do not contribute to light emission, and deteriorate due to non-radiative decay.

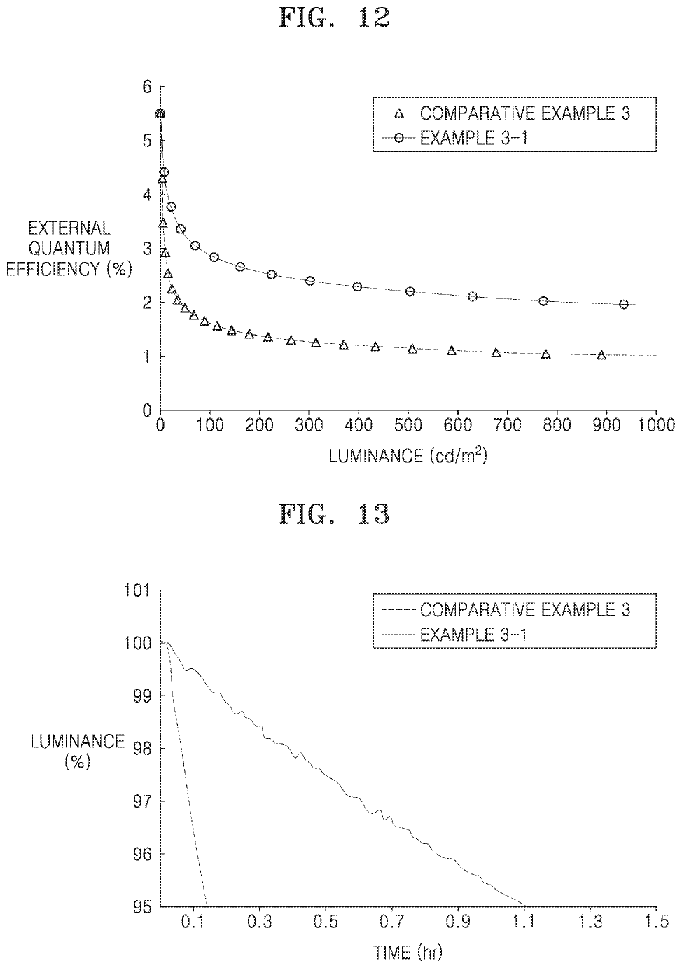

(3) For each of the organic light-emitting devices manufactured according to Comparative Example 3, Example 3-1, and Comparative Example 3-1, external quantum efficiency (EQE) and lifespan were evaluated. Results thereof are shown in FIGS. 12 to 15.

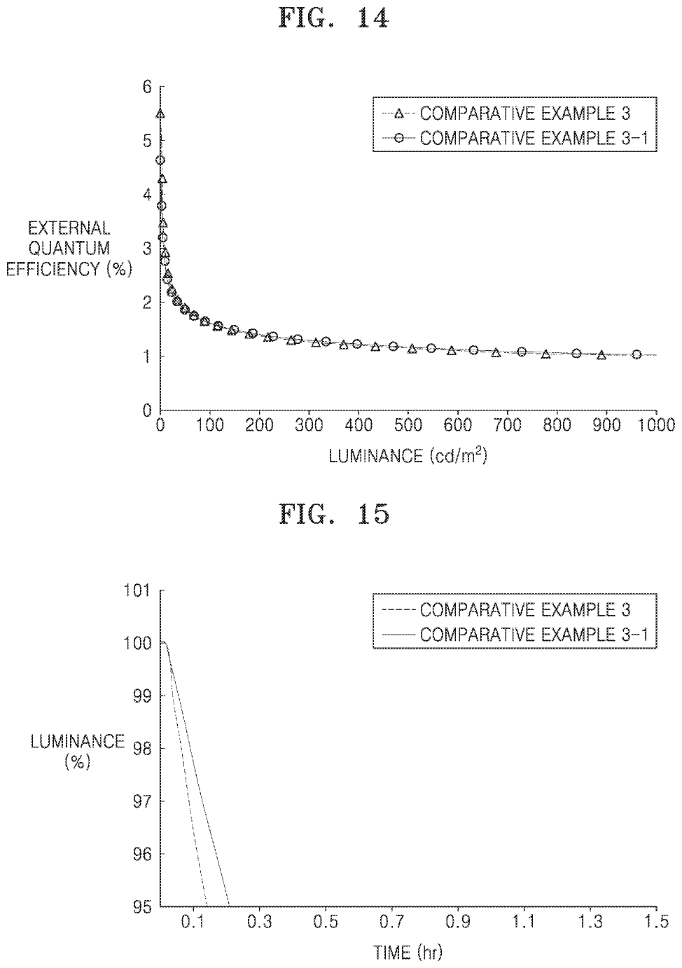

First, referring to FIGS. 12 and 13, in the case of Example 3-1, although some of the triplet excitons generated in SP003, a TADF sensitizer, were lost by FD2, the maximum external quantum efficiency (EQEₘₐₓ, %) was increased compared to Comparative Example 3. Furthermore, instead of minimizing TADF emission, FD2 fluorescence emission may occur, leading to a decrease in the lifespan of excitons. Accordingly, the efficiency roll-off is greatly improved, and at 1000 candela per square meter (cd/m²), which is the actual luminance, EQE was rather increased compared to Comparative Example 3, and as described above, the lifespan was significantly increased.

On the other hand, referring to FIGS. 14 and 15, in the case of Comparative Example 3-1, even compared to Example 3-1 in which the same amount of dopant was included, the external quantum efficiency was significantly decreased, and the increase in the lifespan compared to Comparative Example 3 was negligible compared to Example 3-1. This is because triplet excitons generated in SP002 through dexter energy transfer are transferred to FD-A, and thus, do not contribute to light emission, and deteriorate due to non-radiative decay.

### Evaluation Example 3. HOD Measurement

(1) For Example 1-1, Comparative Example 1, and Comparative Example 1-1, a hole only device (HOD) was fabricated without stacking an electron transport layer and an electron injection layer on an emission layer.

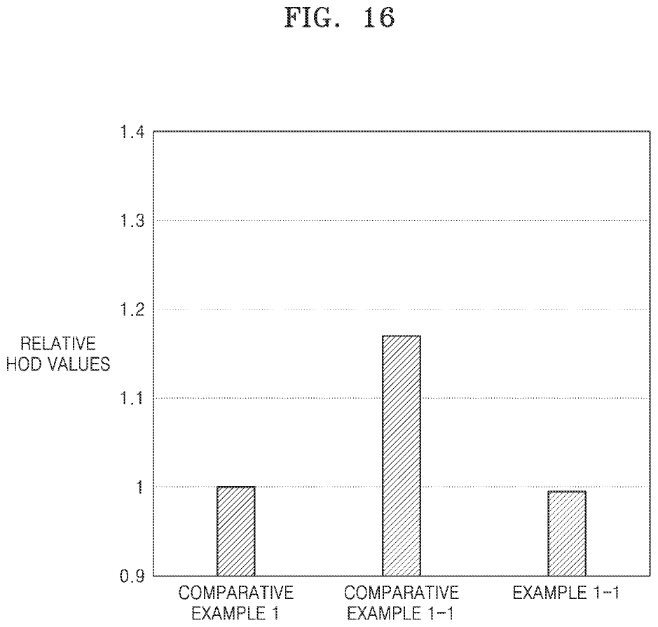

For such a HOD device, a relative HOD value was measured by the method described herein. Results thereof are shown in FIG. 16

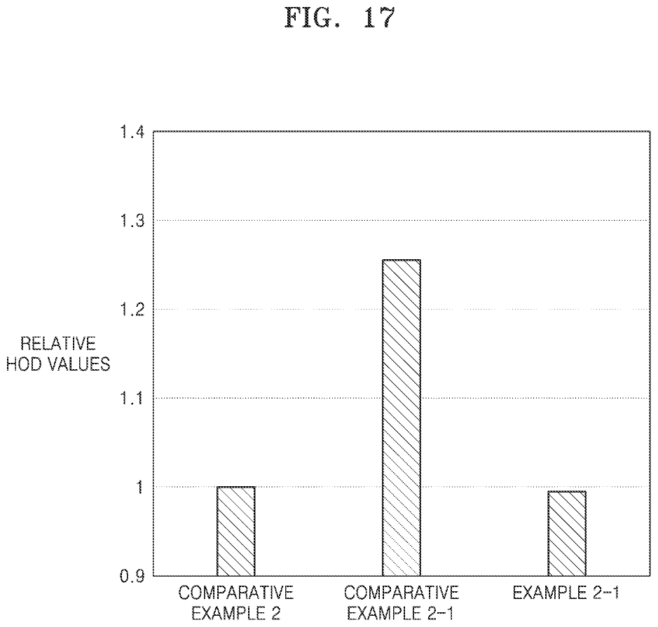

(2) For Example 2-1, Comparative Example 2, and Comparative Example 2-1, a HOD was fabricated in the same manner as in (1), and for such HOD devices, relative HOD values of the HOD devices were measured by the method substrate in this specification. The values are shown in FIG. 17.

Organic light-emitting devices according to one or more embodiments of the present disclosure can have high efficiency and a long lifespan.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments. While one or more exemplary embodiments have been described with reference to the figures, it will be understood by those of ordinary skill in the art that various changes in form and details may be made therein without departing from the spirit and scope as defined by the following claims.

What is claimed is:

1. A composition comprising a first compound, a second compound, and a third compound, wherein
the first compound, the second compound, and the third compound are different from each other,
the first compound satisfies one of Condition 1 and Condition 2, and
the second compound includes a compound represented by Formula 1:
Condition 1
the first compound contains a transition metal
Condition 2
the difference between a triplet energy level of the first compound and a singlet energy level of the first compound is 0.4 eV or less, and the first compound emits delayed fluorescence 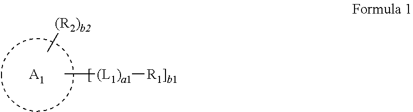
ring A₁ in Formula 1 is a condensed cyclic group in which 3 or more cyclic groups are condensed with each other, and the 3 or more cyclic groups are each a C₅-C₃₀ carbocyclic group or a C₁-C₃₀ heterocyclic group,
L₁ in Formula 1 is a single bond, a C₅-C₃₀carbocyclic group that is unsubstituted or substituted with at least one R₁₀ₐ, or a C₁-C₃₀ heterocyclic group that is unsubstituted or substituted with at least one R₁₀ₐ,
a1 in Formula 1 is an integer from 1 to 5,
b1 in Formula 1 is an integer from 3 to 10, and
R₁ in Formula 1 is a group represented by Formula 2 or Formula 3, 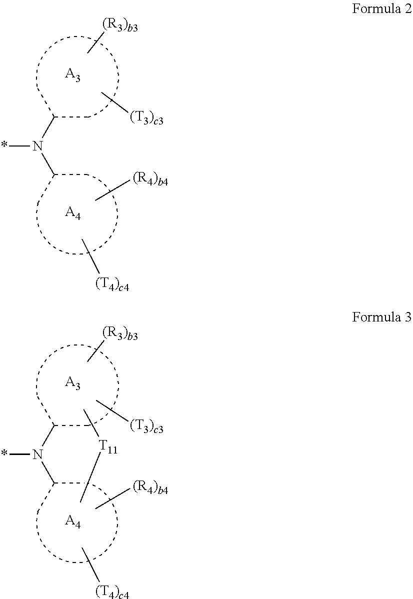
ring A₃ and ring A₄ in Formulae 2 and 3 are each independently a C₅-C₃₀ carbocyclic group or a C₁-C₃₀ heterocyclic group,
T₃ and T₄ in Formulae 2 and 3 are each independently a group represented by *—C(R₅)(R₆)(R₇) or *—Si(R₅)(R₆)(R₇),
c3 and c4 in Formulae 2 and 3 are each independently an integer from 0 to 10, wherein, when c3 is 2 or more, two or more of T₃(s) are identical to or different from each other, and when c4 is 2 or more, two or more of T₄(s) are identical to or different from each other.
T₁₁ in Formula 3 is a single bond, O, S, N(R₈), C(R₈)(R₉), or Si(R₈)(R₉),
R₂ to R₉ and R₁₀ₐ in Formulae 1 to 3 are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₇-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), —P(═S)(Q₈)(Q₉), or —P(Q₈)(Q₉),
b2 to b4 in Formulae 1 to 3 are each independently an integer from 0 to 10, wherein, when b2 is 2 or more, two or more of R₂(s) are identical to or different from each other, when b3 is 2 or more, two or more of R₃(s) are identical to or different from each other, and when b4 is 2 or more, two or more of R₄(s) are identical to or different from each other, and
* indicates a binding site to a neighboring atom,
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(═S)(Q₈)(Q₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(═S)(Q₈)(Q₉), —P(Q₂₈)(Q₂₉), or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), —P(═S)(Q₈)(Q₉), or —P(Q₃₈)(Q₃₉); or
a combination thereof,
wherein Q₁ to Q₉, Q₁₁ to Q₁₉, Q₂₁ to Q₂₉, and Q₃₁ to Q₃₉ are each independently: hydrogen; deuterium; —F; —Cl; —Br; —I; a hydroxyl group; a cyano group; a nitro group; an amidino group; a hydrazine group: a hydrazone group; a carboxylic acid group or a salt thereof; a sulfonic acid group or a salt thereof, a phosphoric acid group or a salt thereof; a C₁-C₆₀ alkyl group which is unsubstituted or substituted with at least one of deuterium, a C₁-C₆₀ alkyl group, a C₆-C₆₀ aryl group, or a combination thereof; a C₂-C₆₀ alkenyl group; a C₂-C₆₀ alkynyl group; a C₁-C₆₀ alkoxy group; a C₁-C₆₀ alkylthio group; a C₃-C₁₀ cycloalkyl group: a C₁-C₁₀ heterocycloalkyl group: a C₃-C₁₀ cycloalkenyl group: a C₁-C₁₀ heterocycloalkenyl group; a C₆-C₆₀ aryl group which is unsubstituted or substituted with at least one of deuterium, a C₁-C₆₀ alkyl group, a C₆-C₆₀ aryl group, or a combination thereof; a C₆-C₆₀ aryloxy group; a C₆-C₆₀ arylthio group; a C₁-C₆₀ heteroaryl group, a C₂-C₆₀ alkyl heteroaryl group; a C₂-C₆₀ heteroaryl alkyl group; a C₁-C₆₀ heteroaryloxy group; a C₁-C₆₀ heteroarylthio group; a monovalent non-aromatic condensed polycyclic group; or a monovalent non-aromatic condensed heteropolycyclic group.

2. The composition of claim 1, wherein ring A₁ in Formula 1 is an anthracene group, a phenalene group, a phenanthrene group, a tetracene group, a pyrene group, a chrysene group, a triphenylene group, a pentacene group, a perylene group, a fluoranthene group, a fluorene group, an acridine group, a phenanthridine group, a phenazine group, a phenoxazine group, a phenothiazine group, a xanthene group, a carbazole group, a dibenzofuran group, or a dibenzothiophene group.

3. The composition of claim 1, wherein the compound represented by Formula 1 is represented by one of Formulae 1-1 to 1-4: 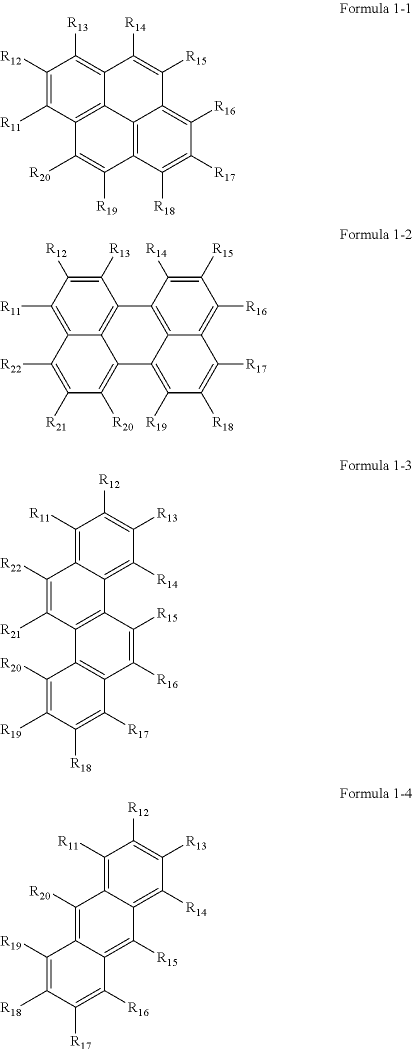
wherein, in Formula 1-1 to 1-4,
R₁₁ to R₂₂ are each independently a group represented by *-(L₁)ₐ₁-R₁, hydrogen deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), —P(═S)(Q₈)(Q₉), or —P(Q₈)(Q₉),
at least three of R₁₁ to R₂₀ in Formulae 1-1 and 1-4 are each independently a group represented by *-(L₁)ₐ₁-R₁,
at least three of R₁₁ to R₂₂ in Formulae 1-2 and 1-3 are each independently a group represented by *-(L₁)ₐ₁-R₁,
*indicates a binding site to a neighboring atom, and
the substituents of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group are as described in claim 1.

4. The compound of claim 3, wherein
i) three or more of R₁₁, R₁₃, R₁₆, and R₁₈ in Formula 1-1 are each independently a group represented by *-(L₁)ₐ₁-R₁;
ii) three or more of R₁₂, R₁₅, R₁₈, and R₂₁ in Formula 1-2 are each independently a group represented by *-(L₁)ₐ₁-R₁;
iii) three or more of R₁₃, R₁₆, R₁₉, and R₂₂ in Formula 1-3 are each independently a group represented by *-(L₁)ₐ₁-R₁; and
iv) three or more of R₁₂, R₁₅, R₁₇, and R₂₀ in Formula 1-4 are each independently a group represented by *-(L₁)ₐ₁-R₁, and
wherein * indicates a binding site to a neighboring atom.

5. The composition of claim 1, wherein the sum of c3 and c4 in Formulae 2 and 3 is 1 or greater.

6. The composition of claim 1, wherein R₅ to R₇ in Formulae 2 and 3 are each independently —CH₃, —CD₃, —CD₂H, —CDH₂, a phenyl group, or a group represented by one of Formulae 9-1 to 9-19: 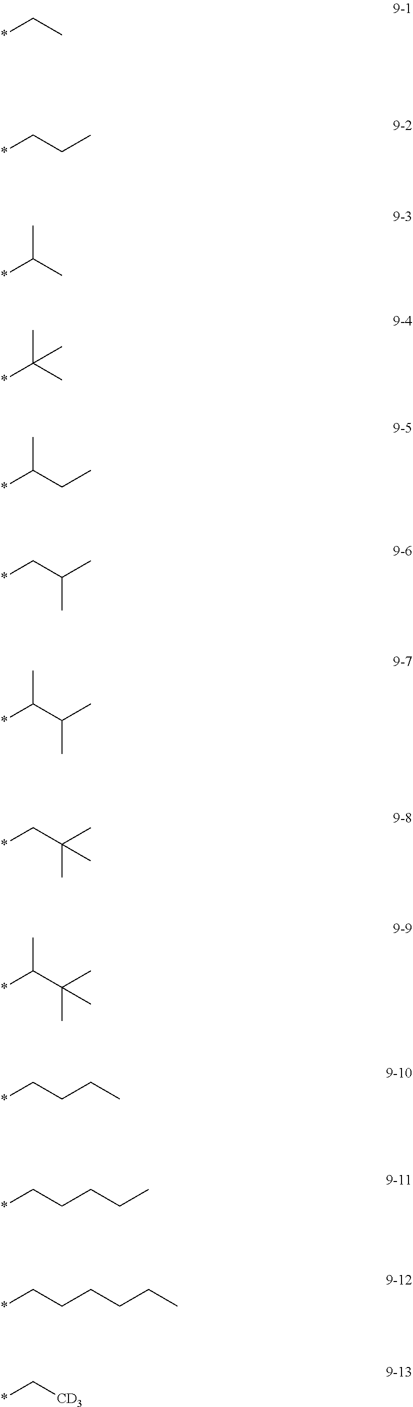 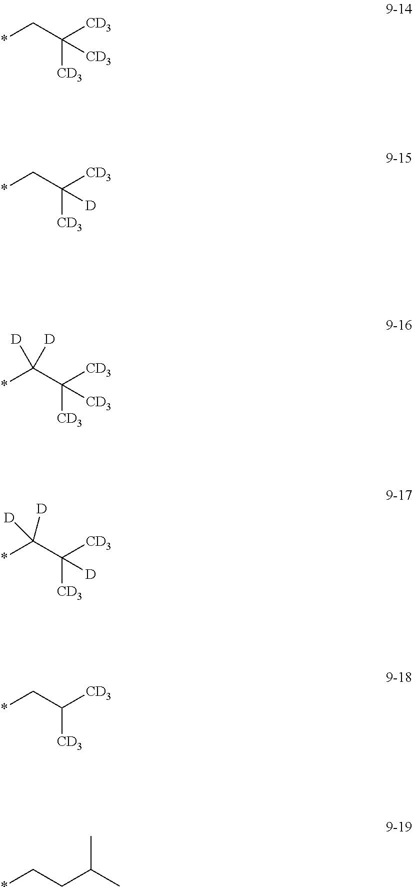
wherein * in Formulae 9-1 to 9-19 indicates a binding site to a neighboring atom.

7. The composition of claim 1, wherein R₁ of Formula 1 is a group represented by one of Formulae 2-1 to 2-42 or 3-1 to 3-36: 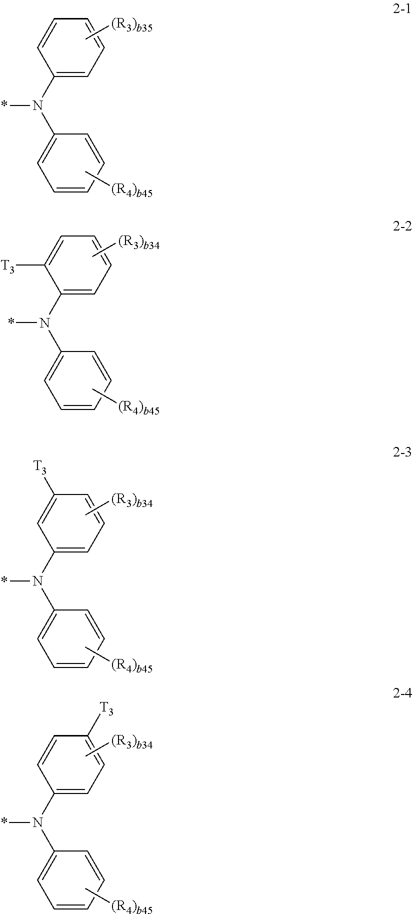 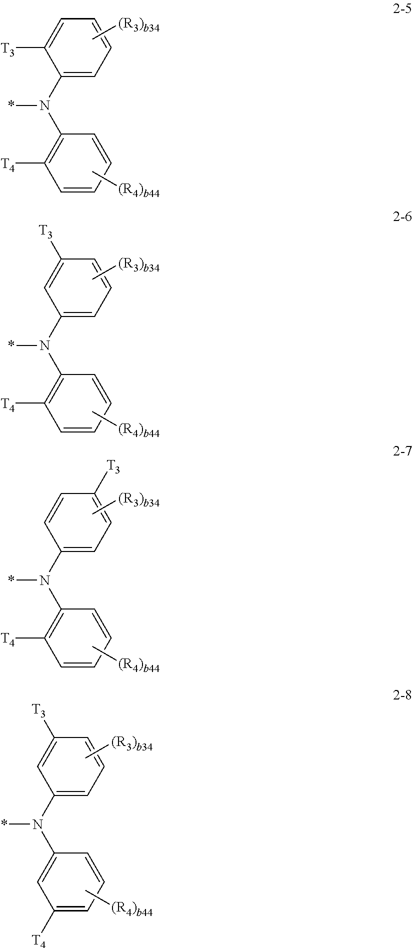 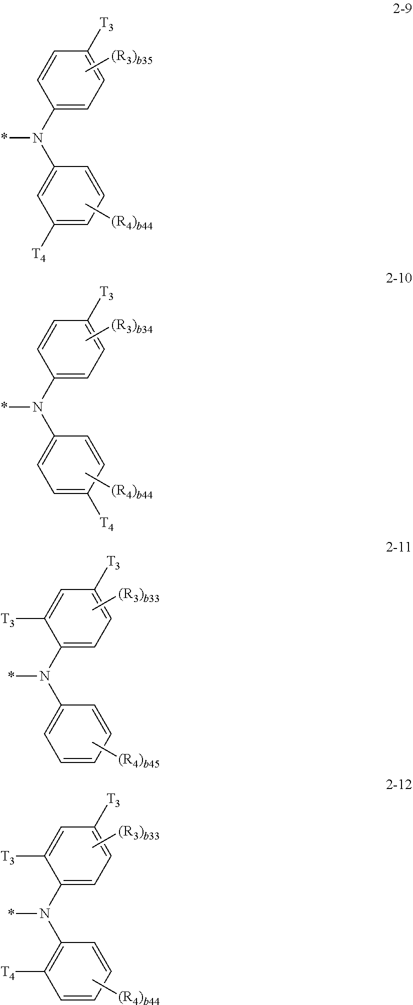 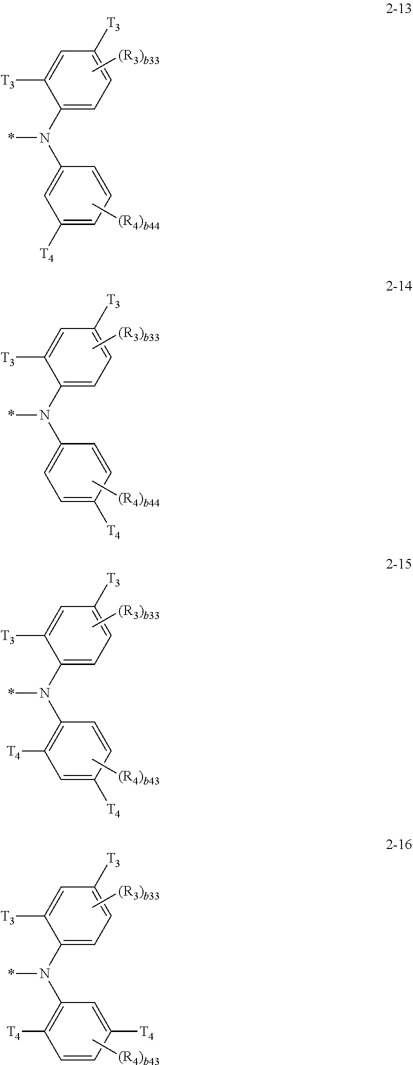 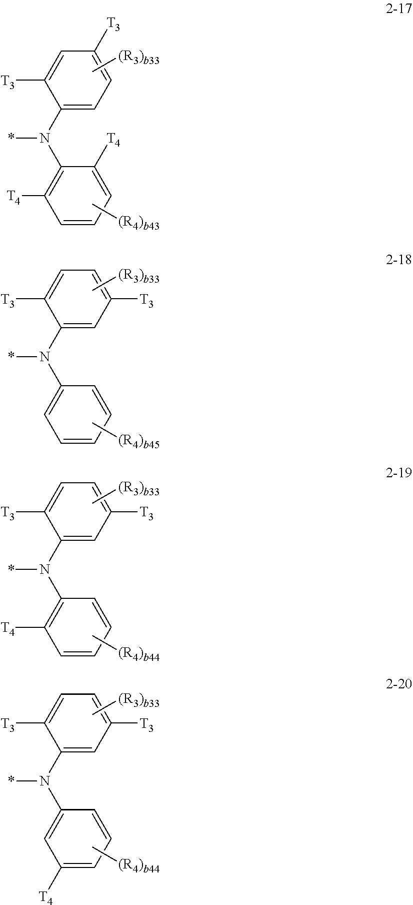 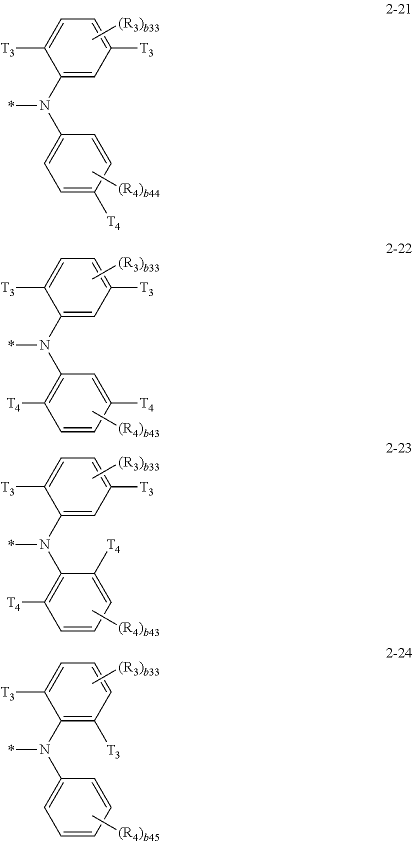 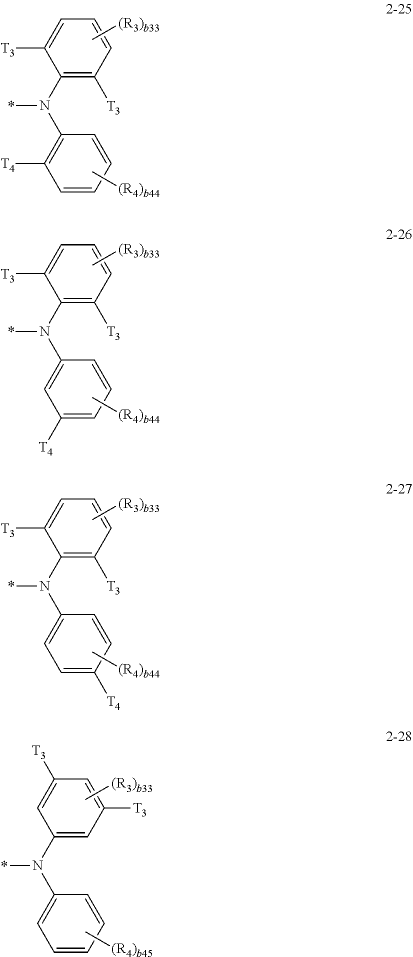 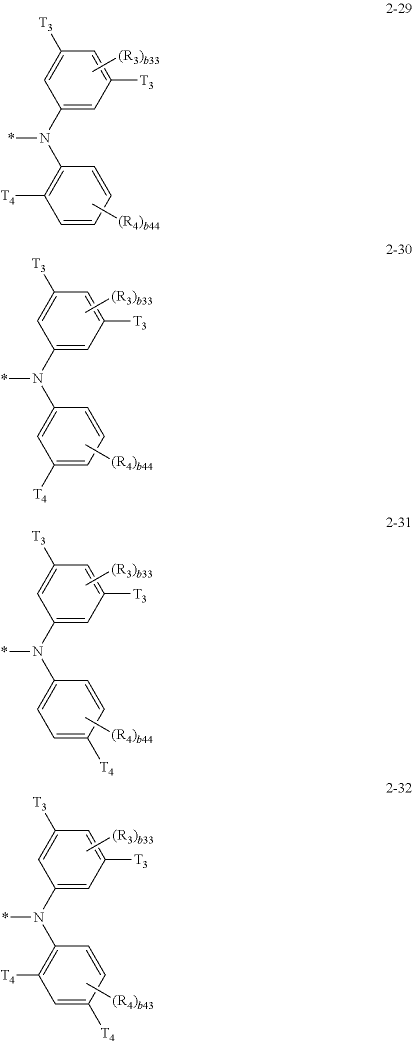 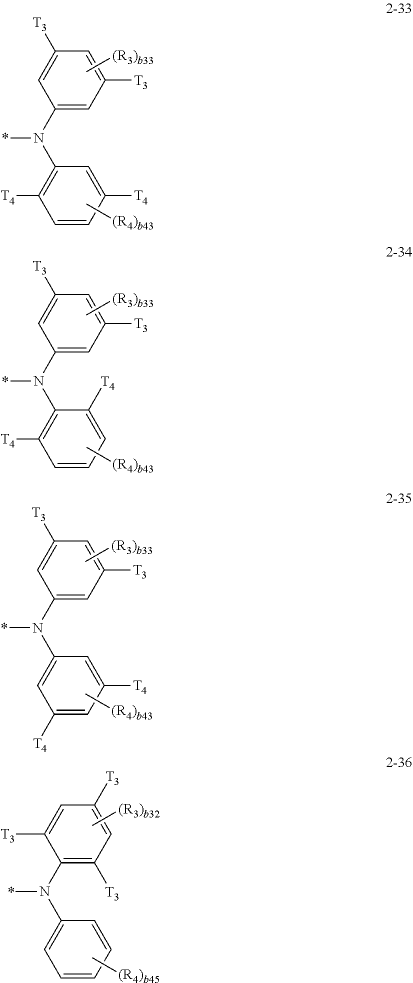 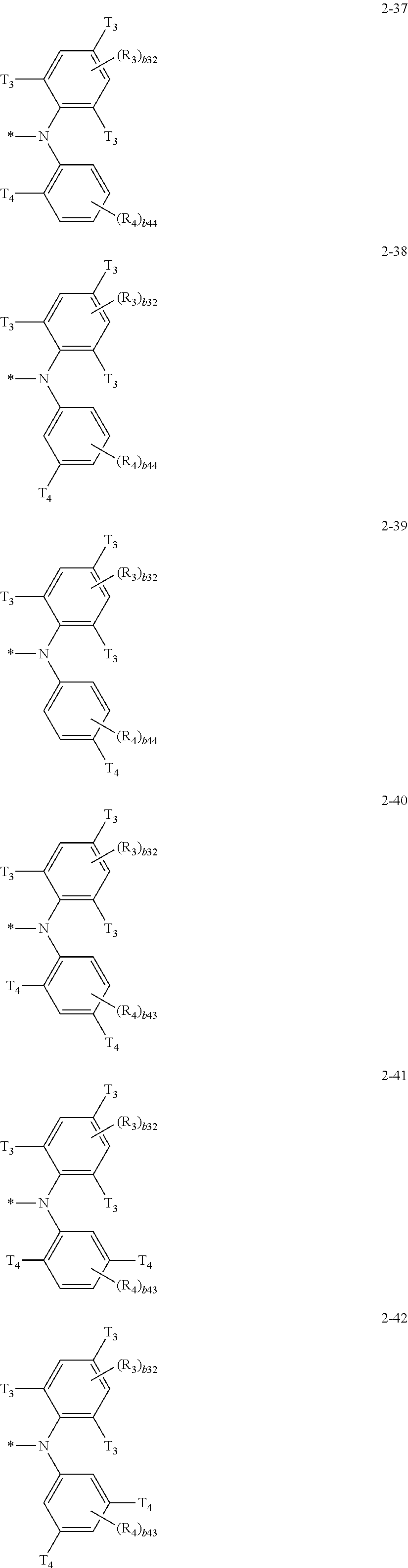 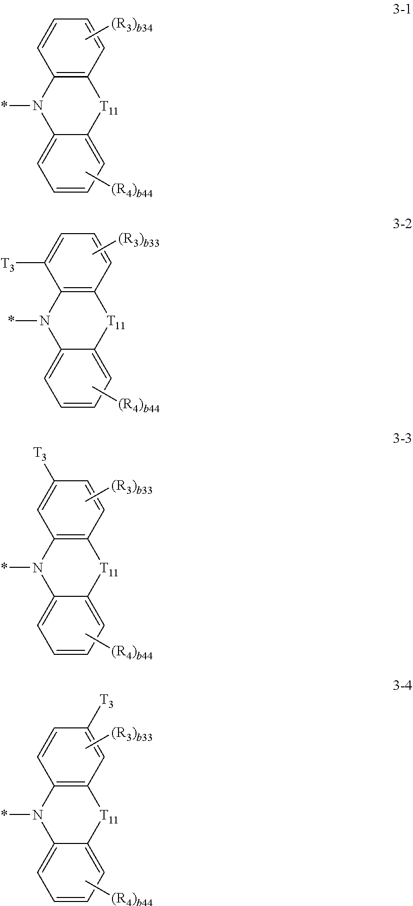 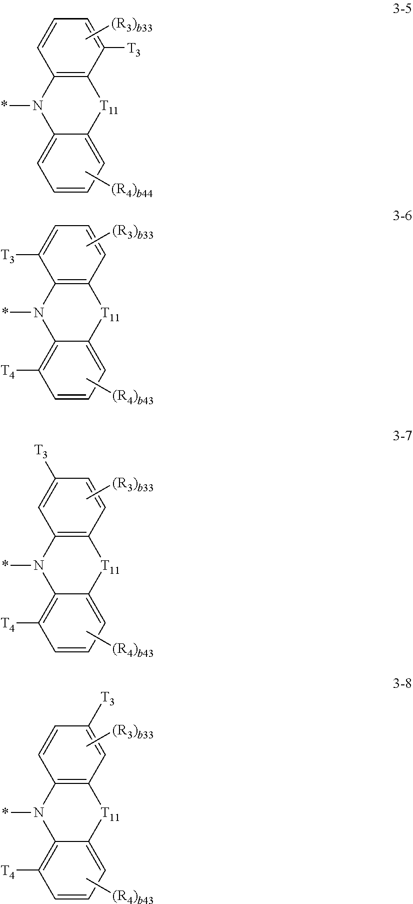 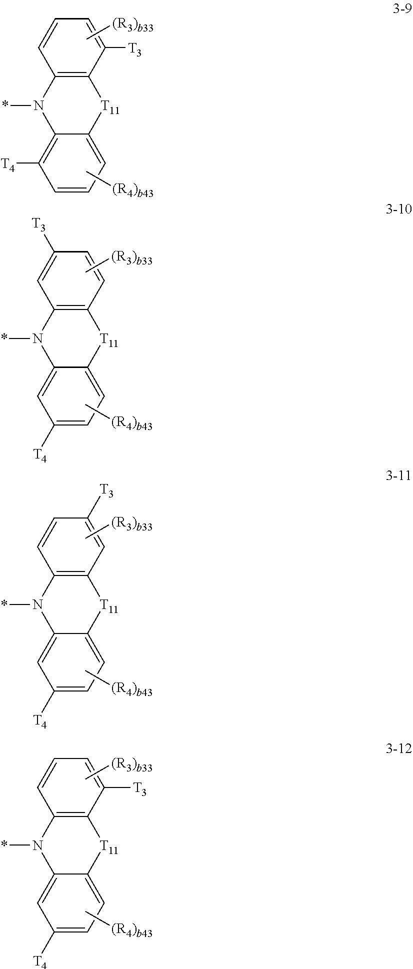 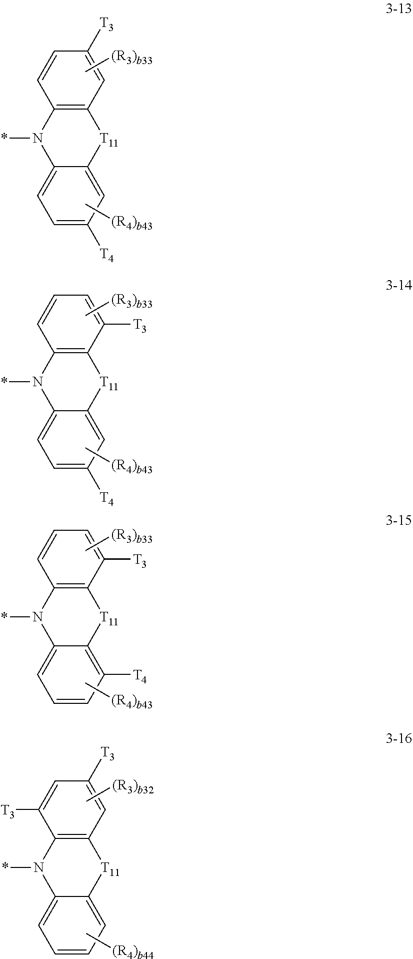 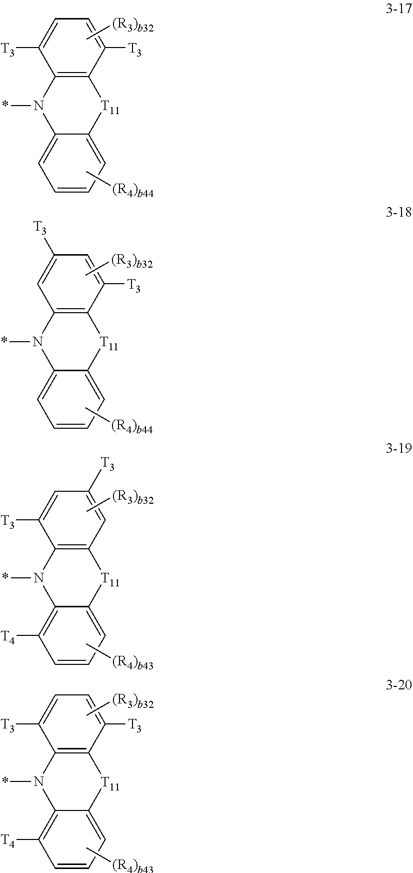 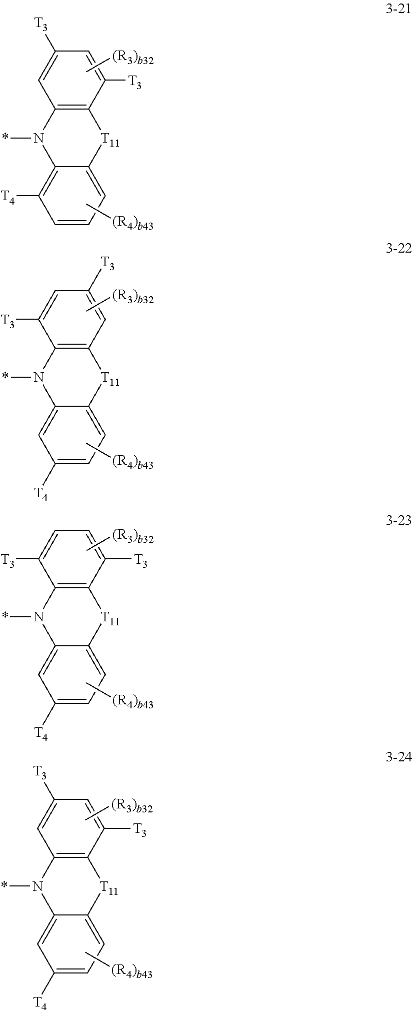 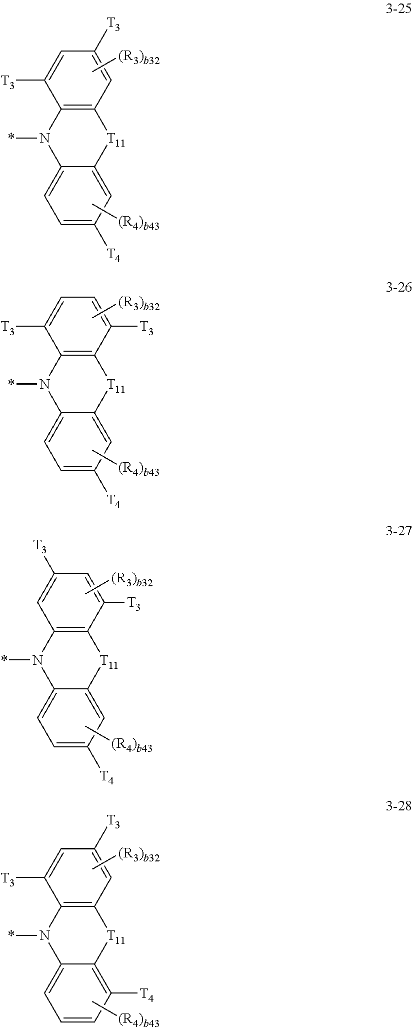 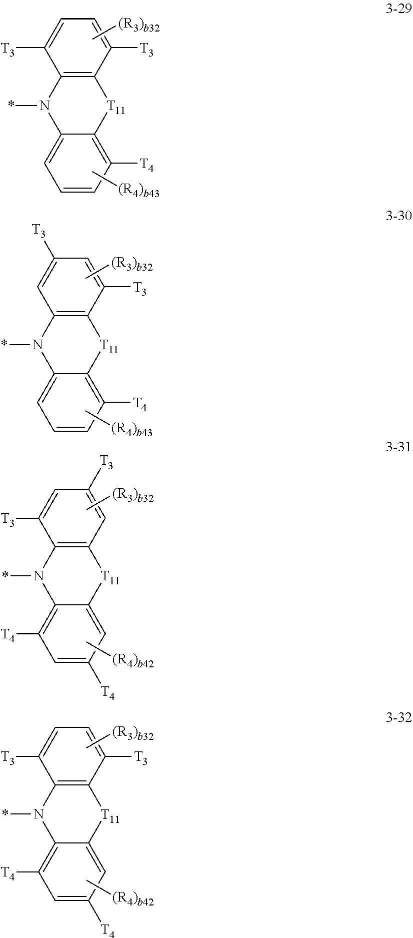 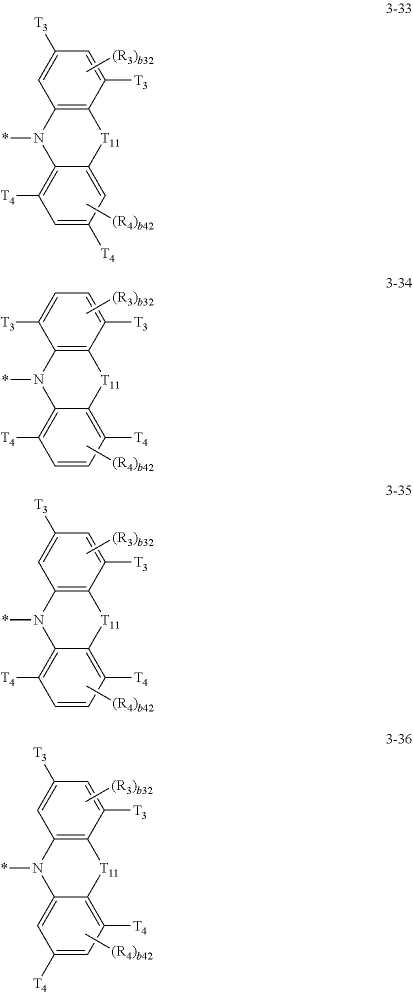
wherein, in Formulae 2-1 to 2-42 and 3-1 to 3-36,
T₃, T₄, T₁₁, R₃, and R₄ are as described in claim 1,
b32 and b42 are each independently an integer from 0 to 2,
b33 and b43 are each independently an integer from 0 to 3,
b34 and b44 are each independently an integer from 0 to 4,
b35 and b45 are each independently an integer from 0 to 5, and
* indicates a binding site to a neighboring atom.

8. The composition of claim 1, wherein the second compound is at least one of Compounds FD1 to FD12:

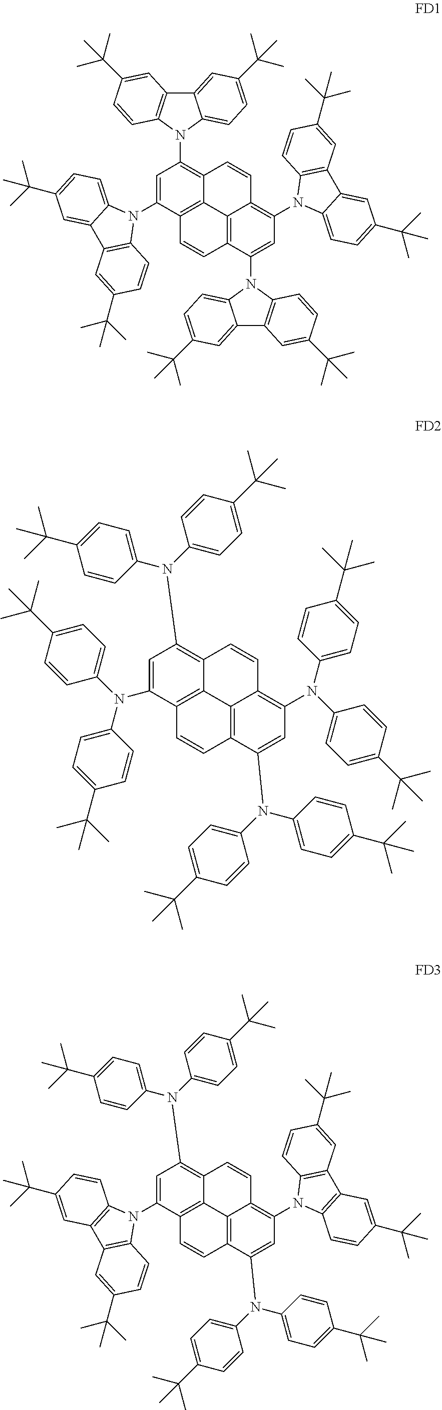

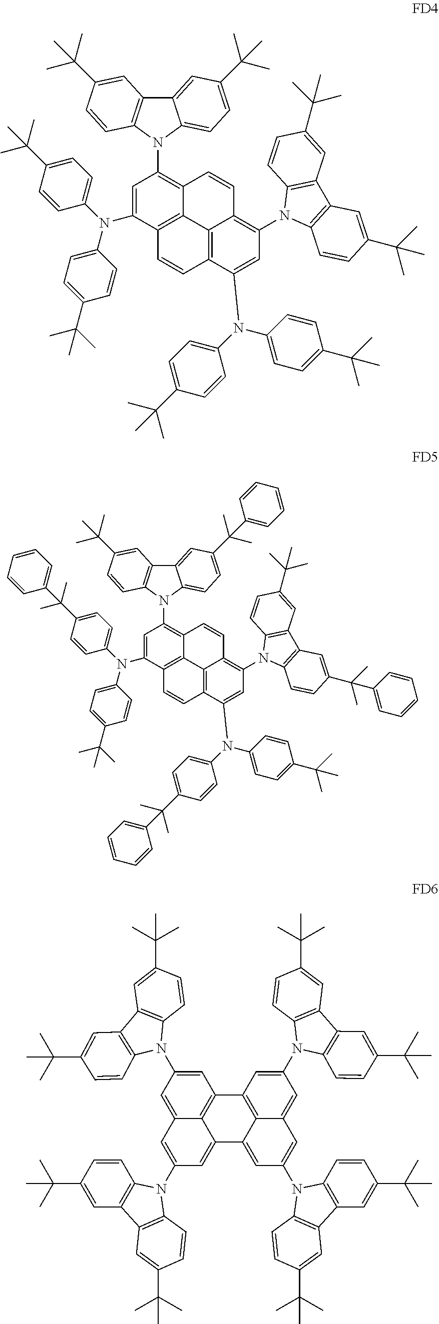

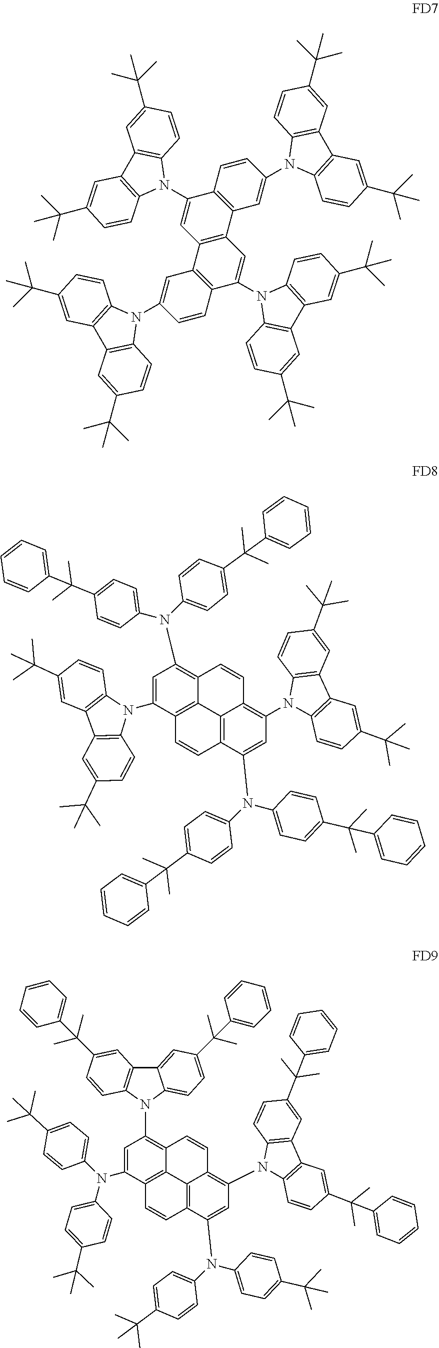

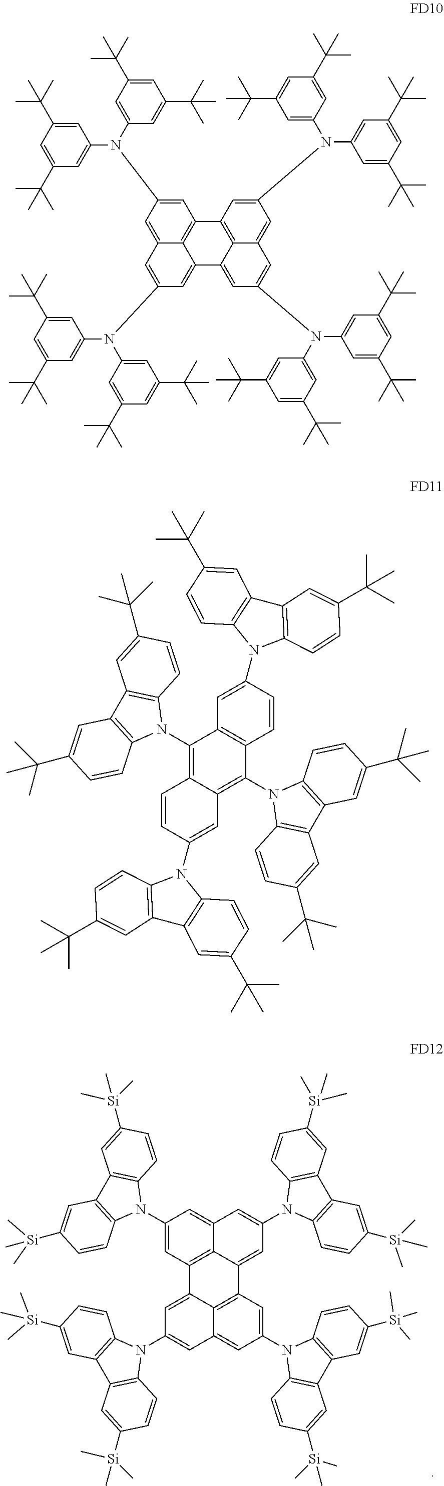

9. The composition of claim 1, wherein the first compound comprises an organometallic compound represented by Formula 101:
M₁₁(L₁₁)ₙ₁₁(L₁₂)ₙ₁₂ Formula 101
wherein, in Formula 101,
M₁₁ is a first-row transition metal of the Periodic Table of Elements, a second-row transition metal of the Periodic Table of Elements, or a third-row transition metal of the Periodic Table of Elements;
L₁₁ is a ligand represented by one of Formulae 101-1 to 101-4;
L₁₂ is a monodentate ligand and a bidentate ligand;
n11 is 1,
n12 is 0, 1, or 2; 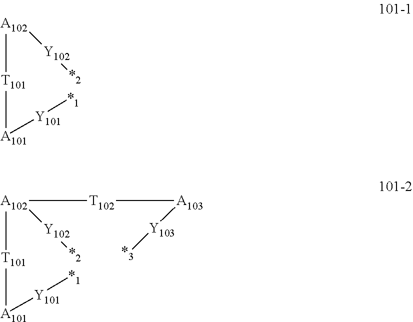 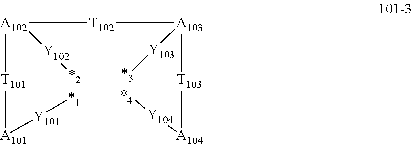 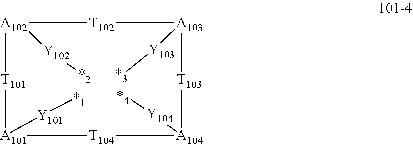
wherein, in Formulae 101-1 to 101-4,
A₁₀₁ to A₁₀₄ are each independently a substituted or unsubstituted C₅-C₃₀ carbocyclic group, a substituted or unsubstituted C₁-C₃₀ heterocyclic group, or a non-cyclic group,
Y₁₀₁ to Y₁₀₄ are each independently a chemical bond, O, S, N(R₉₁), B(R₉₁), P(R₉₁), or C(R₉₁)(R₉₂),
T₁₀₁ to T₁₀₄ are each independently a single bond, a double bond, N(R₉₃)′, B(R₉₃), P(R₉₃), C(R₉₃)(R₉₄), Si(R₉₃)(R₉₄), Ge(R₉₃)(R₉₄), S, Se, O—, C(═O), S(═O), S(═O)₂, —C(R₉₃)═, ═C(R₉₃)—, C(R₉₃)═C(R₉₄), C(═S), or C≡C,
a substituent of the substituted C₅-C₃₀ carbocyclic group, a substituent of substituted C₁-C₃₀ heterocyclic group, and R₉₁ to R₉₄ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —C(═O)(Q₄₁), —S(═O)(Q₄₁), —S(═O)₂(Q₄₁), —N(Q₄₂)(Q₄₃), —B(Q₄₂)(Q₄₃), —Si(Q₄₄)(Q₄₅)(Q₄₆), —Ge(Q₄₄)(Q₄₅)(Q₄₆), —P(═O)(Q₄₇)(Q₄₈), —P(═S)(Q₄₇)(Q₄₈), or —P(Q₄₇)(Q₄₈), wherein each of the substituent of the substituted C₅-C₃₀ carbocyclic group and the substituent of substituted C₁-C₃₀ heterocyclic group is not hydrogen,
*₁, *₂, *₃, and *₄ each indicate a binding site to M₁₁, and
Q₄₁ to Q₄₅ are each independently hydrogen; deuterium: —F, —Cl; —Br; —I; a hydroxyl group; a cyano group; a nitro group; an amidino group, a hydrazine group: a hydrazone group; a C₁-C₆₀ alkyl group; a C₂-C₆₀ alkenyl group; a C₂-C₆₀ alkynyl group; a C₁-C₆₀ alkoxy group; a C₁-C₆₀ alkylthio group; a C₇-C₆₀ aryl alkyl group; a C₃-C₁₀ cycloalkyl group; a C₁-C₁₀ heterocycloalkyl group: a C₃-C₁₀ cycloalkenyl group; a C₁-C₁₀ heterocycloalkenyl group: a C₆-C₆₀ aryl group: a C₇-C₆₀ alkyl aryl group; a C₆-C₆₀ aryloxy group; a C₆-C₆₀ arylthio group; a C₁-C₆₀ heteroaryl group; a C₂-C₆₀ alkyl heteroaryl group; a C₂-C₆₀ heteroaryl alkyl group: a C₁-C₆₀ heteroaryloxy group; a C₁-C₆₀ heteroarylthio group; a monovalent non-aromatic condensed polycyclic group; a monovalent non-aromatic condensed heteropolycyclic group; a C₁-C₆₀ alkyl group substituted with at least one of deuterium, —F, a cyano group, a C₁-C₆₀ alkyl group, or a C₆-C₆₀ aryl group; or a C₆-C₆₀ aryl group substituted with at least one of deuterium, —F, a cyano group, a C₁-C₆₀ alkyl group, or a C₆-C₆₀ aryl group.

10. The composition of claim 1, wherein the first compound includes a thermally activated delayed fluorescence emitter represented by one of Formula 201 or 202: 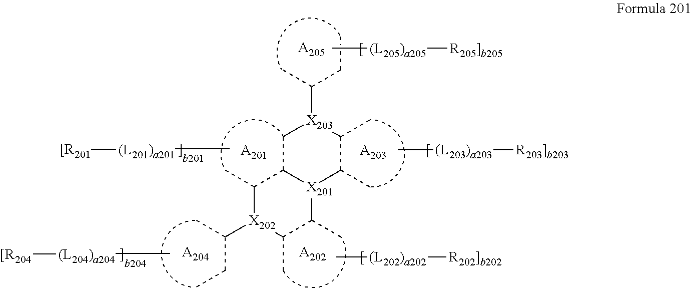
wherein, in Formula 201,
X₂₀₁ to X₂₀₃ are each independently B or N,
A₂₀₁ to A₂₀₅ are each independently a substituted or unsubstituted C₅-C₃₀ carbocyclic group or a substituted or unsubstituted C₁-C₃₀ heterocyclic group,
L₂₀₁ to L₂₀₅ are each independently a single bond, a C₅-C₃₀ carbocyclic group that is unsubstituted or substituted with at least one R₂₀₀ₐ, or a C₁-C₃₀ heterocyclic group that is unsubstituted or substituted with at least one R₂₀₀ₐ,
a201 to a205 are each independently an integer from 1 to 5,
R₂₀₁ to R₂₀₅ and R₂₀₀ₐ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), —P(═S)(Q₅₈)(Q₅₉), or —P(Q₅₈)(Q₅₉),
b201 to b205 are each independently an integer from 0 to 10, wherein, when b201 is 2 or more, two or more of R₂₀₁(s) are identical to or different from each other, when b202 is 2 or more, two or more of R₂₀₂(s) are identical to or different from each other, when b203 is 2 or more, two or more of R₂₀₃(s) are identical to or different from each other, when b204 is 2 or more, two or more of R₂₀₄(s) are identical to or different from each other, and when b205 is 2 or more, two or more of R₂₀₅(s) are identical to or different from each other, 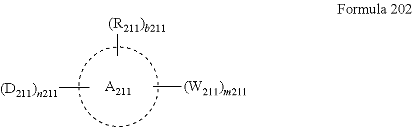
wherein, in Formula 202,
A₂₁₁ is a substituted or unsubstituted C₅-C₃₀carbocyclic group or a substituted or unsubstituted C₁-C₃₀ heterocyclic group,
W₂₁₁ is an acceptor group,
D₂₁₁ is a donor group,
m211 is an integer from 1 to 4, and n211 is an integer from 1 to 4;
R₂₁₁ is hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C_{10 h}eterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), —P(═S)(Q₅₈)(Q₅₉), or —P(Q₅₈)(Q₅₉), and a plurality of R₂₁₁(s) are optionally bonded to form a substituted or unsubstituted C₅-C₃₀ carbocyclic group or a substituted or unsubstituted C₁-C₃₀ heterocyclic group,
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C_{10 h}eterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₆₁)(Q₆₂), —Si(Q₆₃)(Q₆₄)(Q₆₅), —Ge(Q₆₃)(Q₆₄)(Q₆₅), —B(Q₆₆)(Q₆₇), —P(═O)(Q₆₈)(Q₆₉), —P(═S)(Q₆₈)(Q₆₉), —P(Q₆₈)(Q₆₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₇₁)(Q₇₂), —Si(Q₇₃)(Q₇₄)(Q₇₅), —Ge(Q₇₃)(Q₇₄)(Q₇₅), —B(Q₇₆)(Q₇₇), —P(═O)(Q₇₄)(Q₇₉), —P(═S)(Q₇₈)(Q₇₉), —P(Q₇₈)(Q₇₉), or a combination thereof;
—N(Q₈₁)(Q₈₂), —Si(Q₈₃)(Q₈₄)(Q₈₅), —Ge(Q₈₃)(Q₈₄)(Q₈₅), —B(Q₈₆)(Q₈₇), —P(═O)(Q₈₈)(Q₈₉), —P(═O)(Q₇₈)(Q₇₉), or —P(Q₈₈)(Q₈₉); or
a combination thereof,
wherein Q₅₁ to Q₅₉, Q₈₁ to Q₈₉, Q₇₁ to Q₇₉, and Q₈₁ to Q₈₉ are each independently: hydrogen; deuterium; —F; —Cl; —Br; —I; a hydroxyl group; a cyano group; a nitro group: an amidino group; a hydrazine group; a hydrazone group; a carboxylic acid group or a salt thereof; a sulfonic acid group or a salt thereof: a phosphoric acid group or a salt thereof: a C₁-C₆₀ alkyl group which is unsubstituted or substituted with at least one of deuterium, a C₁-C₆₀ alkyl group, a C₆-C₆₀ aryl group, or a combination thereof; a C₂-C₆₀ alkenyl group; a C₂-C₆₀ alkynyl group; a C₁-C₆₀ alkoxy group: a C₁-C₆₀ alkylthio group; a C₃-C₁₀ cycloalkyl group: a C₁-C₁₀ heterocycloalkyl group: a C₃-C₁₀ cycloalkenyl group: a C₁-C₁₀ heterocycloalkenyl group; a C₆-C₆₀ aryl group which is unsubstituted or substituted with at least one of deuterium, a C₁-C₆₀ alkyl group, a C₆-C₆₀ aryl group, or a combination thereof; a C₆-C₆₀ aryloxy group, a C₆-C₆₀ arylthio group; a C₁-C₆₀ heteroaryl group, a C₂-C₆₀ alkyl heteroaryl group; a C₂-C₆₀ heteroaryl alkyl group; a C₁-C₆₀ heteroaryloxy group; a C₁-C₆₀ heteroarylthio group; a monovalent non-aromatic condensed polycyclic group; or a monovalent non-aromatic condensed heteropolycyclic group.

11. The composition of claim 10, wherein W₂₁₁ is a substituted or unsubstituted π electron-deficient nitrogen-free cyclic group, and
D₂₁₁ is:
—F, a cyano group, or a π electron-deficient nitrogen-containing cyclic group;
a C₁-C₆₀ alkyl group, an π-electron deficient nitrogen-containing cyclic group, or an π electron-deficient nitrogen-free cyclic group, each substituted with at least one of —F or a cyano group; or
an π-electron deficient nitrogen-containing cyclic group substituted with at least one of deuterium, a C₁-C₆₀ alkyl group, an π-electron deficient nitrogen-containing cyclic group, or an π electron-deficient nitrogen-free cyclic group,
wherein the π electron-deficient nitrogen-free cyclic group is a phenyl group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, or a condensed cyclic group of two or more π electron-deficient nitrogen-free cyclic groups, and
wherein the π electron-deficient nitrogen-containing cyclic group is a cyclic group having at least one —N=moiety, and is:
an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxalne group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, or a benzimidazolobenzimidazole group; or
a condensed cyclic group in which two or more π electron-efficient nitrogen-containing cyclic groups are condensed with each other.

12. The composition of claim 1, wherein the third compound comprises a bipolar compound, an electron-transporting compound, a hole-transporting compound, or a combination thereof,
the electron-transporting compound comprises at least one electron-transporting moiety,
the hole-transporting compound does not comprise the electron-transporting moiety, and
the electron-transporting moiety is a cyano group, a π electron-deficient nitrogen-containing cyclic group, or a group represented by one of the following Formulae: 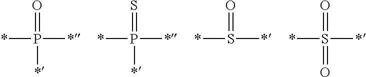
wherein *, *′, and *″ in the formulae above are each a binding site to a neighboring atom.

13. The composition of claim 12, wherein
the electron-transporting compound includes at least one π electron-deficient nitrogen-free cyclic group and at least one electron-transporting moiety,
the hole-transporting compound includes at least one π electron-deficient nitrogen-free cyclic group, and does not include an electron-transporting moiety, and
the electron-transporting moiety is a cyano group or a π electron-deficient nitrogen-containing cyclic group.

14. The composition of claim 12, wherein
the electron-transporting compound comprises i) at least one of a cyano group, a pyrimidine group, a pyrazine group, or a triazine group, or ii) a triphenylene group, and
the hole-transporting compound comprises a carbazole group.

15. The composition of claim 1, wherein the composition satisfies Condition 3:
E_{HOMO_C1}−04eV≤E_{HOMO_C2}≤E_{HOMO_C1}+0.1eV Condition 3
wherein, in Condition 3, E_{HOMO_C1} is a highest occupied molecular orbital (HOMO) energy level value of the first compound, E_{HOMO_C2} is a HOMO energy level value of the second compound, and eV is electron volts.

16. The composition of claim 1, wherein the composition satisfies Condition 4:
R(HOD)_{H}/R(HOD)₀≤1.07 Condition 4
wherein, in Condition 4,
R(HOD)_{H} is a relative HOD value of the composition comprising the first compound, the second compound, and the third compound, and
R(HOD)₀ is a relative HOD value of a composition comprising the first compound and the third compound.

17. An organic light-emitting device comprising:
a first electrode;
a second electrode; and
an organic layer located between the first electrode and the second electrode,
wherein the organic layer comprises an emission layer, and
wherein the organic layer comprises the composition of claim 1.

18. The organic light-emitting device of claim 17, wherein the emission layer comprises the composition.

19. The organic light-emitting device of claim 18, wherein the emission layer comprises a sensitizer, a dopant, and a host,
the sensitizer comprises the first compound of the composition,
the dopant comprises the second compound of the composition, and
the host comprises the third compound of the composition.

20. The organic light-emitting device of claim 18, wherein the emission layer emits blue light.

21. An organic light-emitting device, comprising:
a first electrode;
a second electrode;
m light-emitting units located between the first electrode and the second electrode and comprising at least one emission layer; and
m−1 charge generation layers located between neighboring two light-emitting units of the m light-emitting units, and comprising an n-type charge generation layer and a p-type charge generation layer,
wherein m is an integer of 2 or greater,
a maximum emission wavelength of light emitted from at least one light-emitting unit of the m light-emitting units is different from a maximum emission wavelength of light emitted from at least one light-emitting unit of the other light-emitting units of the m light-emitting units, and
the at least one emission layer comprises the composition of claim 1.

22. An organic light-emitting device, comprising:
a first electrode;
a second electrode; and
m emission layers located between the first electrode and the second electrode,
wherein,
m is an integer of 2 or more,
a maximum emission wavelength of light emitted from at least one emission layer of the m emission layers is different from a maximum emission wavelength of light emitted from at least one emission layer of the other emission layers of the m emission layers, and
the at least one emission layer comprises the composition of claim 1.
